(19) United States
(12) Patent Application Publication
(10) Pub. No.: US 2023/0172066 A1
(43) Pub. Date: Jun. 1, 2023

(54) LIGHT EMITTING ELEMENT AND AMINE COMPOUND FOR THE SAME

(71) Applicant: Samsung Display Co., Ltd., Yongin-si (KR)

(72) Inventor: Takuya UNO, Yokohama (JP)

(21) Appl. No.: 18/060,288

(22) Filed: Nov. 30, 2022

(30) Foreign Application Priority Data
Dec. 1, 2021 (KR) 10-2021-0170078
Nov. 16, 2022 (KR) 10-2022-0153783

(51) Int. Cl. H10K 85/60; C07D 211/54; C07D 405/12; C07D 409/12; C07D 405/14; C07D 409/14; H10K 50/18

(52) U.S. Cl. CPC H01L 51/0072; C07D 211/54; H01L 51/006; H01L 51/0052; H01L 51/0073; C07D 405/12; H01L 51/0074; C07D 409/12; H01L 51/0061; H01L 51/0059; C07D 405/14; C07D 409/14; H01L 51/5096; C07B 2200/05

(57) ABSTRACT

A light emitting element and an amine compound for the light emitting element are provided. The light emitting element includes a first electrode, a second electrode on the first electrode, and at least one functional layer between the first electrode and the second electrode The functional layer includes the amine compound and the luminous efficiency and service life of the light emitting element is improved.

### CROSS-REFERENCE TO RELATED APPLICATION

This application claims priority to and the benefit of Korean Patent Application No. 10-2021-0170078, filed on Dec. 01, 2021, and Korean Patent Application No. 10-2022-0153783, filed Nov. 16, 2022, the entire contents of both of which are hereby incorporated by reference.

### BACKGROUND

### 1. Field

Aspects of one or more embodiments of the present disclosure herein relate to an amine compound and a light emitting element including the same, and for example, to a light emitting element including an amine compound in a hole transport region.

### 2. Description of the Related Art

Recently, the development of an organic electroluminescence display device as an image display device is being actively conducted. The organic electroluminescence display device includes a self-luminescent light emitting element in which holes and electrons injected from a first electrode and a second electrode recombine in an emission layer, and thus, a luminescent material of the emission layer emits light to implement display.

In the application of a light emitting element to a display device, there is a demand for a light emitting element having low driving voltage, high luminous efficiency, and a long service life, and development on materials for a light emitting element capable of stably attaining such characteristics is being continuously required (sought).

In addition, development on materials of a hole transport region for suppressing the diffusion of exciton energy of the emission layer is being carried out in order to implement a light emitting element with high efficiency.

### SUMMARY

Aspects of one or more embodiments of the present disclosure are directed to a light emitting element exhibiting excellent (suitable) luminous efficiency and long service life characteristics.

Embodiments of the present disclosure also provide an amine compound as a material for a light emitting element having high efficiency and long service life characteristics.

An embodiment of the present disclosure provides an amine compound represented by Formula 1:

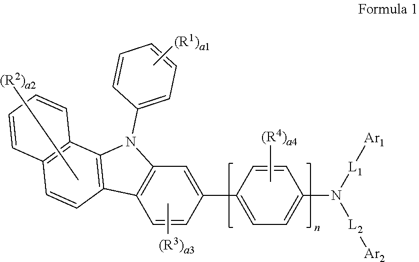

In Formula 1, R¹ to R⁴ may each independently be a hydrogen atom or a deuterium atom, a1 may be an integer of 0 to 5, a2 may be an integer from 0 to 6, a3 may be an integer from 0 to 3, a4 may be an integer from 0 to 4, n may be 1 or 2, L₁ and L₂ may each independently be a direct linkage, a substituted or unsubstituted arylene group having 6 to 30 ring-forming carbon atoms, or a substituted or unsubstituted heteroarylene group having 5 to 30 ring-forming carbon atoms, Ar₁ and Ar₂ may each independently be a substituted or unsubstituted aryl group having 6 to 40 ring-forming carbon atoms, a substituted or unsubstituted heteroaryl group having 5 to 40 ring-forming carbon atoms, or at least one selected from among Formula 2 to Formula 5, and at least one selected from Ar₁ and Ar₂ is at least one selected from among Formula 2 to Formula 5.

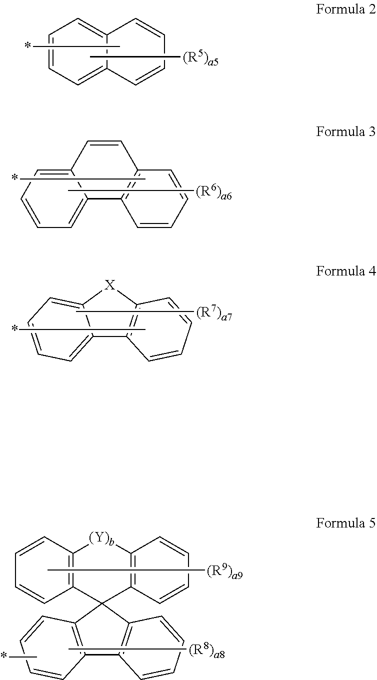

In Formula 2 to Formula 5, [symbol] may be a part bonded to L₁ or L₂ in Formula 1, R⁵ to R⁹ may each independently be a hydrogen atom, a deuterium atom, a halogen atom, a substituted or unsubstituted aryl group having 6 to 30 ring-forming carbon atoms, a substituted or unsubstituted heteroaryl group having 5 to 30 ring-forming carbon atoms, a substituted or unsubstituted alkyl group having 1 to 30 carbon atoms, or a substituted or unsubstituted alkenyl group having 2 to 30 carbon atoms, or may be bonded to an adjacent group to form a ring, a5, a7, and a8 may each independently be an integer from 0 to 7, a6 may be an integer from 0 to 9, and a9 may be an integer from 0 to 8, in Formula 4, X may be O or S, and in Formula 5, b may be 0 or 1, and when b is 1, Y is a direct linkage.

In Formula 1, when one selected from among Ar₁ and Ar₂ is Formula 2, the other is Formula 2 or Formula 3, when one selected from among Ar₁ and Ar₂ is Formula 3, the other is a substituted or unsubstituted aryl group having 6 to 40 ring-forming carbon atoms, a substituted or unsubstituted heteroaryl group having 5 to 40 ring-forming carbon atoms, or one selected from among Formula 3 to Formula 5, when one selected from among Ar₁ and Ar₂ is Formula 4, the other is a substituted or unsubstituted aryl group having 6 to 40 ring-forming carbon atoms, a substituted or unsubstituted heteroaryl group having 5 to 40 ring-forming carbon atoms, or one selected from among Formula 4 and Formula 5, or when one selected from among Ar₁ and Ar₂ is Formula 5, the other is a substituted or unsubstituted aryl group having 6 to 40 ring-forming carbon atoms, or a substituted or unsubstituted heteroaryl group having 5 to 40 ring-forming carbon atoms.

In an embodiment, Formula 1 may be represented by Formula 1-1:

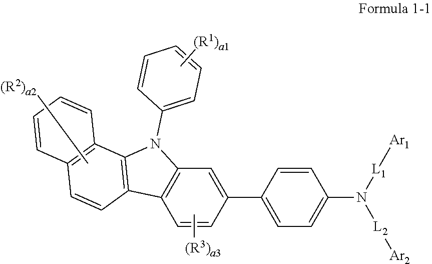

In Formula 1-1, R¹ to R³, a1 to a3, L₁, L₂, Ar₁, and Ar₂ may each independently be the same as defined in Formula 1.

In an embodiment, in Formula 1 and Formula 1-1, L₁ and L₂ may each be independently a direct linkage, or a substituted or unsubstituted arylene group having 6 to 30 ring-forming carbon atoms.

In an embodiment, Formula 2 may be represented by one selected from among 2-1 and 2-2:

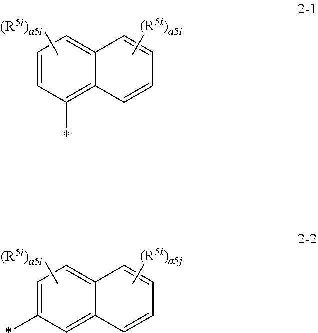

In 2-1 and 2-2, R⁵ⁱ and R^{5j} may each independently be a hydrogen atom, a deuterium atom, or a substituted or unsubstituted aryl group having 6 to 30 ring-forming carbon atoms, a5i may be an integer from 0 to 3, and a5j may be an integer from 0 to 4.

In an embodiment, Formula 3 e may be represented by one selected from among 3-1 to 3-3:

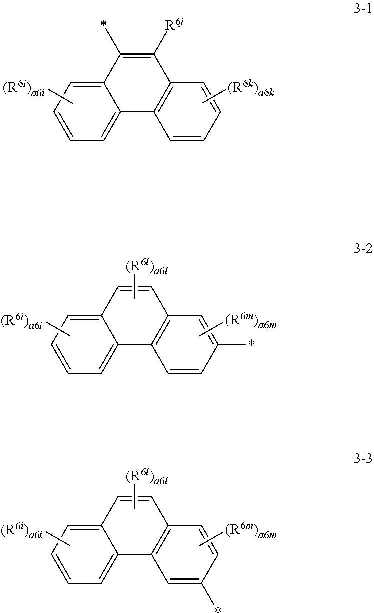

In 3-1 to 3-3, R⁶ⁱ, _{R}^{6j}, R^{6k}, R^{6l}, R^{6k} and R^{6m} may each independently be a hydrogen atom, a deuterium atom, or a substituted or unsubstituted aryl group having 6 to 30 ring-forming carbon atoms, and a6i may be an integer from 0 to 4, in 3-1, a6k may be an integer from 0 to 4, and in 3-2 and 3-3, a61 may be an integer from 0 to 2, and a6m may be an integer from 0 to 3.

In an embodiment, Formula 4 may be represented by one selected from among 4-1 to 4-4:

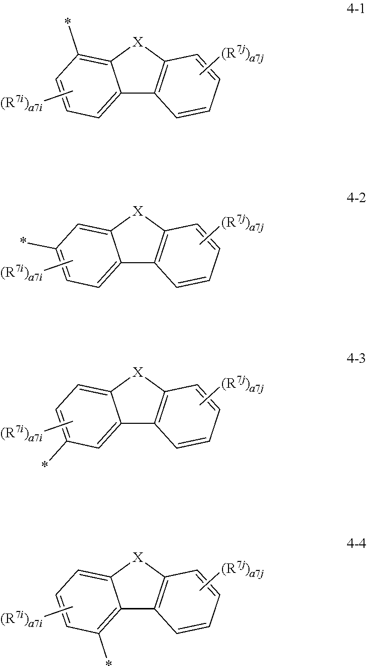

In 4-1 to 4-4, R⁷ⁱ and R^{7j} may each independently be a hydrogen atom, a deuterium atom, or a substituted or unsubstituted aryl group having 6 to 30 ring-forming carbon atoms, or are bonded to an adjacent group to form a ring, a7i may be an integer from 0 to 3, and a7j may be an integer from 0 to 4.

In an embodiment, Formula 5 may be represented by one selected from among 5-1 to 5-6:

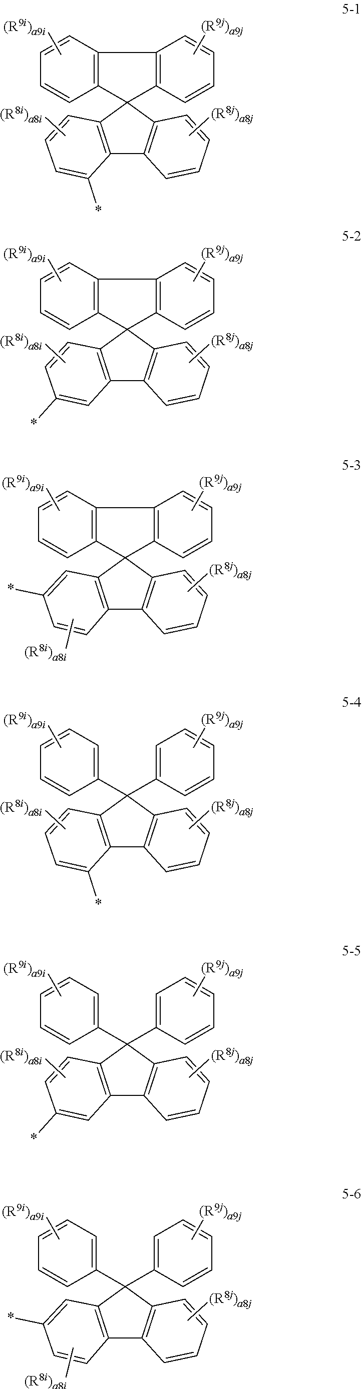

In 5-1 to 5-6, R⁸ⁱ, R^{8j}, R⁹ⁱ and R^{9j} may each independently be a hydrogen atom, a deuterium atom, or a substituted or unsubstituted aryl group having 6 to 30 ring-forming carbon atoms, or may be bonded to an adjacent group to form a ring, a8i may be an integer from 0 to 3, and a8j, a9i and a9j may each independently be an integer from 0 to 4.

In an embodiment of the present disclosure, a light emitting element may include: a first electrode; a second electrode on the first electrode; and at least one functional layer between the first electrode and the second electrode and includes the above-described amine compound.

In an embodiment, the at least one functional layer may include an emission layer, a hole transport region between the first electrode and the emission layer, and an electron transport region between the emission layer and the second electrode, and the hole transport region may include the amine compound.

In an embodiment, the hole transport region may include at least one selected from a hole injection layer, a hole transport layer, and an electron blocking layer, and at least one selected from the hole injection layer, the hole transport layer, and the electron blocking layer may include the amine compound.

In an embodiment, the emission layer may include a compound represented by Formula E-1:

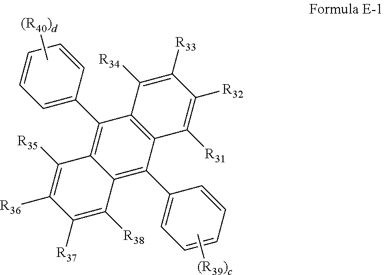

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring, and c and d may each independently be an integer from 0 to 5.

### BRIEF DESCRIPTION OF THE DRAWINGS

The above and other aspects and features of certain embodiments of the disclosure will be more apparent from the following description taken in conjunction with the accompanying drawings, in which:

FIG. 1 is a plan view illustrating a display device according to an embodiment;

FIG. 2 is a cross-sectional view illustrating a part taken along line I-I′ of FIG. 1;

FIG. 3 is a cross-sectional view schematically illustrating a light emitting element according to an embodiment;

FIG. 4 is a cross-sectional view schematically illustrating a light emitting element according to an embodiment;

FIG. 5 is a cross-sectional view schematically illustrating a light emitting element according to an embodiment;

FIG. 6 is a cross-sectional view schematically illustrating a light emitting element according to an embodiment;

FIG. 7 is a cross-sectional view illustrating a display device according to an embodiment;

FIG. 8 is a cross-sectional view illustrating a display device according to an embodiment;

FIG. 9 is a cross-sectional view illustrating a display device according to an embodiment; and

FIG. 10 is a cross-sectional view illustrating a display device according to an embodiment.

### DETAILED DESCRIPTION

The present disclosure may have one or more suitable modifications and may be embodied in different forms, and example embodiments will be explained in more detail with reference to the accompany drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure.

In the present disclosure, when a component (or a region, a layer, a portion, etc.) is referred to as being “on,” “connected to,” or “coupled to” another component, the component may be directly disposed on/connected to/coupled to the other component, or that a third component may be disposed therebetween.

Like reference numerals refer to like components throughout, and duplicative descriptions thereof may not be provided. Also, in the drawings, the thicknesses, ratios, and dimensions of the components may be exaggerated for effective description of technical contents. The term “and/or” includes all combinations of one or more of which associated configurations may define.

It will be understood that, although the terms “first,” “second,” etc. may be used herein to describe one or more suitable components, these components should not be limited by these terms. These terms are only used to distinguish one component from another. For example, a first component could be termed a second component, and, similarly, a second component could be termed a first component, without departing from the scope of the present disclosure. As used herein, the singular forms, “a,” “an,” and “the” are intended to include the plural forms as well, unless the context clearly indicates otherwise.

In some embodiments, terms such as “below,” “under,” “on,” and “above” may be used to describe the relationship between components illustrated in the drawings. The terms are used as a relative concept and are described with reference to the direction indicated in the drawings.

It should be understood that the terms “comprise,” or “have” are intended to specify the presence of stated features, integers, steps, operations, components, parts, or combinations thereof in the disclosure, but do not preclude the presence or addition of one or more other features, integers, steps, operations, components, parts, or combinations thereof.

Unless otherwise defined, all terms (including technical and scientific terms) used herein have the same meaning as commonly understood by one of ordinary skill in the art to which the present disclosure belongs. In addition, it will be understood that terms, such as those defined in commonly used dictionaries, should be interpreted as having a meaning that is consistent with their meaning in the context of the relevant art and will not be interpreted in an idealized or overly formal sense unless expressly so defined herein.

In the present application, when a part such as a layer, a film, a region, or a plate is referred to as being “on” or “above” another part, it can be directly on the other part, or an intervening part may also be present. In contrast, when a part such as a layer, a film, a region, or a plate is referred to as being “under” or “below” another part, it can be directly under the other part, or an intervening part may also be present. In addition, in the present disclosure, it will be understood that when a part is referred to as being “on” another part, it may be disposed on an upper portion of the another part, or disposed on a lower portion of the another part as well.

In the disclosure, the term “substituted or unsubstituted” may refer to being substituted or unsubstituted with at least one substituent selected from the group including (e.g., consisting of) a deuterium atom, a halogen atom, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the substituents exemplified above may be substituted or unsubstituted. For example, a biphenyl group may be interpreted as an aryl group or a phenyl group substituted with a phenyl group.

In the disclosure, the phrase “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In addition, the rings formed by being bonded to each other may be connected to another ring to form a spiro structure.

In the disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In addition, two methyl groups in 4,5-dimethylphenanthrene may be interpreted as “adjacent groups” to each other.

In the disclosure, examples of the halogen atom may include a fluorine atom, a chlorine atom, a bromine atom, or an iodine atom.

In the disclosure, the alkyl group may be a linear, branched or cyclic type or kind. The number of carbons in the alkyl group may be 1 to 60, 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but the embodiment of the present disclosure is not limited thereto.

The hydrocarbon ring group herein refers to a functional group or substituent derived from an aliphatic hydrocarbon ring or a ring in which an aliphatic hydrocarbon ring group and an aromatic hydrocarbon ring are fused. The number of ring-forming carbon atoms in the hydrocarbon ring group may be 5 to 60, 5 to 30, or 5 to 30.

In the disclosure, an aryl group refers to a functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but the embodiment of the present disclosure is not limited thereto.

In the disclosure, the heterocyclic group refers to a functional group or substituent derived from a ring containing at least one selected from B, O, N, P, Se, Si, and S as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

If the heterocyclic group includes two or more heteroatoms, the two or more heteroatoms may be the same as or different from each other. The heterocyclic group may be a monocyclic heterocyclic group or a polycyclic heterocyclic group and includes a heteroaryl group. The number of ring-forming carbon atoms in the heterocyclic group may be 2 to 60, 2 to 30, 2 to 20, or 2 to 10.

In the disclosure, the aliphatic heterocyclic group may contain at least one selected from B, O, N, P, Se, Si, and S as a heteroatom. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group etc., but the embodiment of the present disclosure is not limited thereto.

In the disclosure, the heteroaryl group may contain at least one selected from B, O, N, P, Se, Si, and S as a heteroatom. When the heteroaryl group contains two or more heteroatoms, the two or more heteroatoms may be the same as or different from each other. The heteroaryl group may be a monocyclic heteroaryl group or polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but the embodiment of the present disclosure is not limited thereto.

In the disclosure, the above description of the aryl group may be applied to an arylene group except that the arylene group is a divalent group. The above description of the heteroaryl group may be applied to a heteroarylene group except that the heteroarylene group is a divalent group.

In the disclosure, the fluorenyl group may be substituted, and two substituents may be bonded to each other to form a spiro structure. Examples of embodiments in which the fluorenyl group is substituted are as follows. However, the embodiment of the present disclosure is not limited thereto.

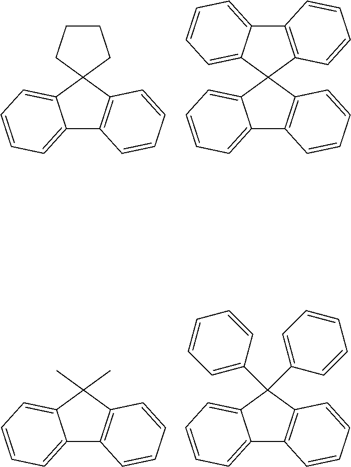

In the disclosure, a silyl group includes an alkyl silyl group and/or an aryl silyl group. Examples of the silyl group may include a trimethylsilyl group, a triethylsilyl group, a t-butyldimethylsilyl group, an ethyldimethylsilyl group, a propyldimethylsilyl group, a triphenylsilyl group, a diphenylsilyl group, a phenylsilyl group, etc., but the embodiment of the present disclosure is not limited thereto.

In the disclosure, a thio group may include an alkylthio group and/or an arylthio group. The thio group may refer to a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the embodiment of the present disclosure is not limited thereto.

In the disclosure, an oxy group may refer to an oxygen atom that is bonded to the alkyl group or the aryl group as defined above. The oxy group may include an alkoxy group and an aryl oxy group. The alkoxy group may be a linear chain, a branched chain or a ring chain. The number of carbon atoms in the alkoxy group is not specifically limited, but may be, for example, 1 to 60, 1 to 20 or 1 to 10. The number of ring-forming carbon atoms in the aryloxy group is not specifically limited, but may be, for example, 6 to 60, 6 to 30 or 6 to 20. Examples of the oxy group include methoxy, ethoxy, n-propoxy, isopropoxy, butoxy, pentyloxy, hexyloxy, octyloxy, nonyloxy, decyloxy, benzyloxy, etc., but the embodiment of the present disclosure is not limited thereto.

The boron group herein may refer to a boron atom that is bonded to the alkyl group or the aryl group as defined above. The boron group includes an alkyl boron group and/or an aryl boron group. Examples of the boron group may include a dimethylboron group, a diethylboron group, a t-butylmethylboron group, a diphenylboron group, a phenylboron group, etc., but the embodiment of the present disclosure is not limited thereto.

In the disclosure, the number of carbon atoms in an amine group is not specifically limited, but may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, a triphenylamine group, etc., but the embodiment of the present disclosure is not limited thereto.

In the disclosure, the alkyl groups in an alkylthio group, an alkylsulfoxy group, an alkylaryl group, an alkylamino group, an alkyl boron group, an alkyl silyl group, and/or an alkyl amine group may be the same as the examples of the alkyl group described above.

In the disclosure, the aryl group in an aryloxy group, an arylthio group, an arylsulfoxy group, an arylamino group, an arylboron group, an arylsilyl group, and/or an arylamine group may be the same as the examples of the aryl group described above.

In the disclosure, a direct linkage may mean a single bond. In some embodiment, in the disclosure,

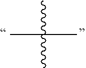

and “ [symbol] ” mean a position to be connected.

Hereinafter, a light emitting element according to an embodiment of the present disclosure will be described with reference to the drawings.

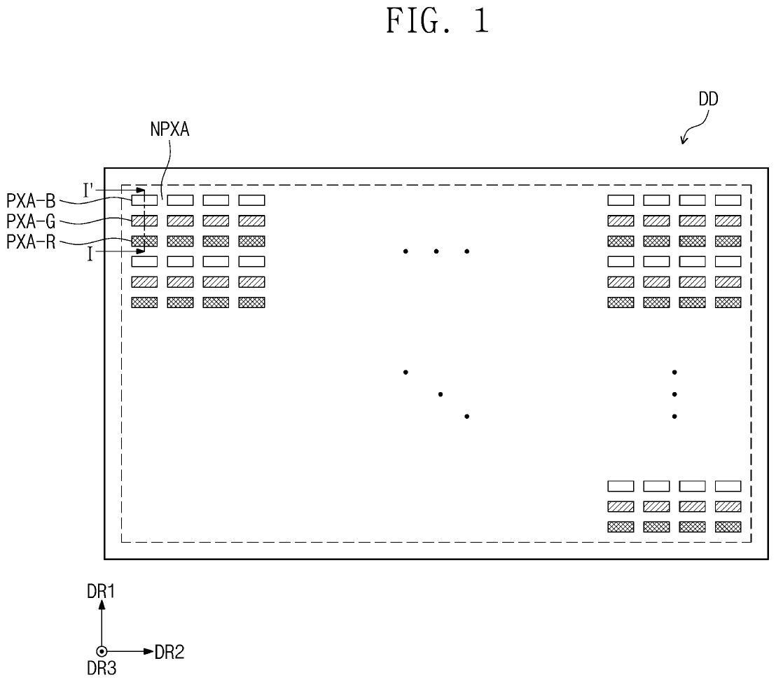

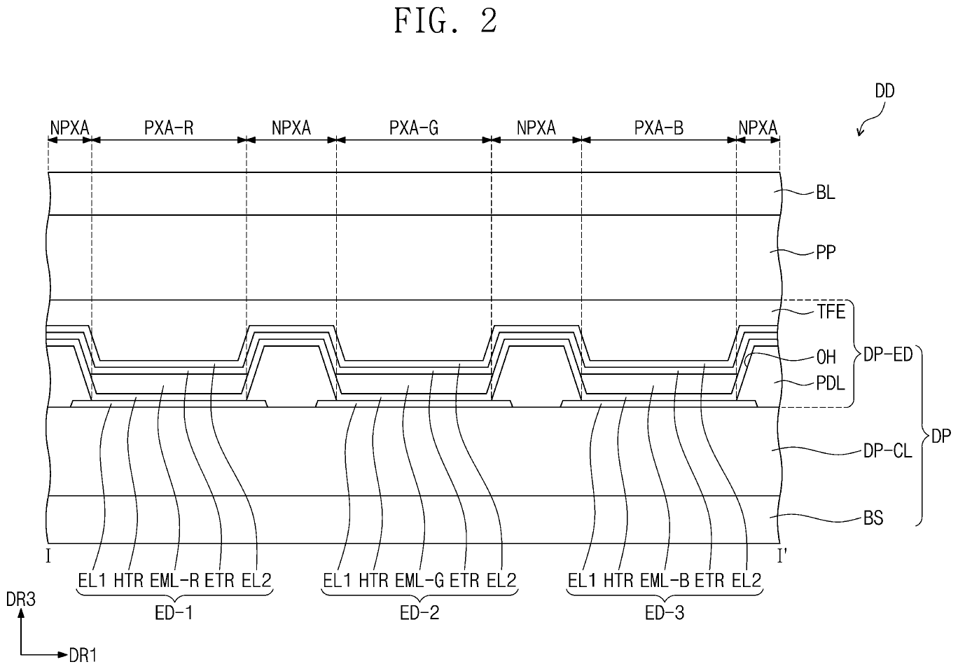

FIG. 1 is a plan view illustrating an embodiment of a display device DD. FIG. 2 is a cross-sectional view of the display device DD of the embodiment. FIG. 2 is a cross-sectional view illustrating a part taken along line I-I′ of FIG. 1.

The display device DD may include a display panel DP and an optical layer PP disposed on the display panel DP. The display panel DP may include light emitting elements ED-1, ED-2, and ED-3. The display device DD may include a plurality of light emitting elements ED-1, ED-2, and ED-3. The optical layer PP may be disposed on the display panel DP and control reflected light in the display panel DP due to external light. The optical layer PP may include, for example, a polarization layer or a color filter layer. In some embodiment, the optical layer PP may not be provided in the display device DD.

A base substrate BL may be on the optical layer PP. The base substrate BL may be a member which provides a base surface on which the optical layer PP is disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In addition, in an embodiment, the base substrate BL may not be provided.

The display device DD according to an embodiment may further include a filling layer. The filling layer may be between a display element layer DP-ED and the base substrate BL. The filling layer may be an organic material layer. The filling layer may include at least one selected from an acrylic-based resin, a silicone-based resin, and an epoxy-based resin.

The display panel DP may include a base layer BS, a circuit layer DP-CL provided on the base layer BS, and the display element layer DP-ED. The display element layer DP-ED may include a pixel defining film PDL, the light emitting elements ED-1, ED-2, and ED-3 disposed between portions of the pixel defining film PDL, and an encapsulation layer TFE disposed on the light emitting elements ED-1, ED-2, and ED-3.

The base layer BS may be a member which provides a base surface on which the display element layer DP-ED is disposed. The base layer BS may be a glass substrate, a metal substrate, a plastic substrate, etc. However, the embodiment is not limited thereto, and the base layer BS may be an inorganic layer, an organic layer, or a composite material layer.

In an embodiment, the circuit layer DP-CL is disposed on the base layer BS, and the circuit layer DP-CL may include a plurality of transistors. Each of the transistors may include a control electrode, an input electrode, and an output electrode. For example, the circuit layer DP-CL may include a switching transistor and a driving transistor in order to drive the light emitting elements ED-1, ED-2, and ED-3 of the display element layer DP-ED.

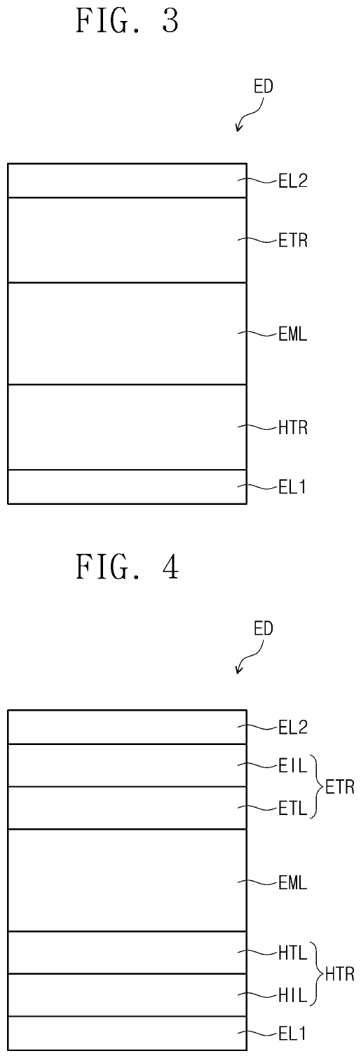

Each of the light emitting elements ED-1, ED-2, and ED-3 may have a structure of a light emitting element ED according to FIGS. 3 to 6. Each of the light emitting elements ED-1, ED-2 and ED-3 may include a first electrode EL1, a hole transport region HTR, emission layers EML-R, EML-G and EML-B, an electron transport region ETR, and a second electrode EL2.

FIG. 2 illustrates an embodiment in which the emission layers EML-R, EML-G, and EML-B of the light emitting elements ED-1, ED-2, and ED-3 are disposed in openings OH defined in the pixel defining film PDL, and the hole transport region HTR, the electron transport region ETR, and the second electrode EL2 are provided as a common layer in the entire light emitting elements ED-1, ED-2, and ED-3. However, the embodiment of the present disclosure is not limited thereto, and the hole transport region HTR and the electron transport region ETR in an embodiment may be provided by being patterned inside the openings OH defined in the pixel defining film PDL. For example, the hole transport region HTR, the emission layers EML-R, EML-G, and EML-B, and the electron transport region ETR of the light emitting elements ED-1, ED-2, and ED-3 in an embodiment may be provided by being patterned in an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be formed by laminating one layer or a plurality of layers. The encapsulation layer TFE may include at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film may protect (reduce) the display element layer DP-ED from moisture/oxygen, and the encapsulation-organic film may protect (reduce) the display element layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not limited thereto. The encapsulation-organic film may include an acrylic-based compound, an epoxy-based compound, and/or the like. The encapsulation-organic film may include a photopolymerizable organic material, but the embodiment of the present disclosure is not limited thereto.

The encapsulation layer TFE may be disposed on the second electrode EL2 and may be disposed while filling the opening OH.

Referring to FIGS. 1 and 2, the display device DD may include a non-light emitting region NPXA and light emitting regions PXA-R, PXA-G and PXA-B. The light emitting regions PXA-R, PXA-G and PXA-B may be regions in which light generated by the respective light emitting elements ED-1, ED-2 and ED-3 is emitted. The light emitting regions PXA-R, PXA-G, and PXA-B may be spaced apart from each other on a plane (e.g., in a plan view).

Each of the light emitting regions PXA-R, PXA-G, and PXA-B may be a region divided by the pixel defining film PDL. The non-light emitting regions NPXA may be regions between the adjacent light emitting regions PXA-R, PXA-G, and PXA-B, which correspond to portions of the pixel defining film PDL. In some embodiments, in the disclosure, the light emitting regions PXA-R, PXA-G, and PXA-B may respectively correspond to pixels. The pixel defining film PDL may divide the light emitting elements ED-1, ED-2, and ED-3. The emission layers EML-R, EML-G and EML-B of the light emitting elements ED-1, ED-2 and ED-3 may be disposed in openings OH defined in the pixel defining film PDL and separated from each other.

The light emitting regions PXA-R, PXA-G and PXA-B may be divided into a plurality of groups according to the color of light generated from the light emitting elements ED-1, ED-2 and ED-3. In the display device DD shown in FIGS. 1 and 2, three light emitting regions PXA-R, PXA-G, and PXA-B which emit red light, green light, and blue light, respectively are illustrated. For example, the display device DD may include the red light emitting region PXA-R, the green light emitting region PXA-G, and the blue light emitting region PXA-B that are separated from each other.

In the display device DD according to an embodiment, the plurality of light emitting elements ED-1, ED-2 and ED-3 may emit light beams having wavelengths different from each other. For example, in an embodiment, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2, and ED-3 may emit light beams in the same wavelength range or at least one light emitting element may emit a light beam in a wavelength range different from the others. For example, the first to third light emitting elements ED-1, ED-2, and ED-3 may all emit blue light.

The light emitting regions PXA-R, PXA-G, and PXA-B in the display device DD according to an embodiment may be arranged in a stripe form. Referring to FIG. 1, the plurality of red light emitting regions PXA-R may be arranged with each other along a second direction axis DR2, the plurality of green light emitting regions PXA-G may be arranged with each other along the second direction axis DR2, and the plurality of blue light emitting regions PXA-B each may be arranged with each other along the second direction axis DR2. In some embodiments, the red light emitting region PXA-R, the green light emitting region PXA-G, and the blue light emitting region PXA-B may be alternately arranged in this order along a first direction axis DR1. (DR3 is a third direction which is normal or perpendicular to the plane defined by the first direction DR1 and the second direction DR2).

FIGS. 1 and 2 illustrate that all the light emitting regions PXA-R, PXA-G, and PXA-B have substantially similar areas, but the embodiment of the present disclosure is not limited thereto. Thus, the light emitting regions PXA-R, PXA-G, and PXA-B may have different areas from each other according to the wavelength range of the emitted light. In this embodiment, the areas of the light emitting regions PXA-R, PXA-G, and PXA-B may refer to areas when viewed on a plane defined by the first directional axis DR1 and the second directional axis DR2.

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display device DD. For example, the arrangement form of the light emitting regions PXA-R, PXA-G, and PXA-B may be a PENTILE® arrangement form (for example, an RGBG matrix, an RGBG structure, or RGBG matrix structure) or a Diamond Pixel™ arrangement form. PENTILE® is a duly registered trademark of Samsung Display Co., Ltd. Diamond Pixel™ is the atoms of Samsung's OLED displays, consisting of red, blue, and green (RGB) screen dots in the shape of diamonds.

In some embodiments, the areas (i.e., sizes) of the light emitting regions PXA-R, PXA-G, and PXA-B may be different from each other. For example, in an embodiment, the area of the green light emitting region PXA-G may be smaller than that of the blue light emitting region PXA-B, but the embodiment of the present disclosure is not limited thereto.

Hereinafter, FIGS. 3 to 6 are cross-sectional views schematically illustrating light emitting elements according to embodiments. Each of the light emitting elements ED according to embodiments may include a first electrode EL1, a second electrode EL2 facing the first electrode EL1, and at least one functional layer disposed between the first electrode EL1 and the second electrode EL2. Each of the light emitting elements ED of embodiments may include an amine compound of an embodiment, described below, in at least one functional layer.

Each of the light emitting elements ED may include, as at least one functional layer, a hole transport region HTR, an emission layer EML, and an electron transport region ETR that are sequentially stacked. Referring to FIG. 3, the light emitting element ED may include a first electrode EL1, a hole transport region HTR, an emission layer EML, an electron transport region ETR, and a second electrode EL2 that are sequentially stacked.

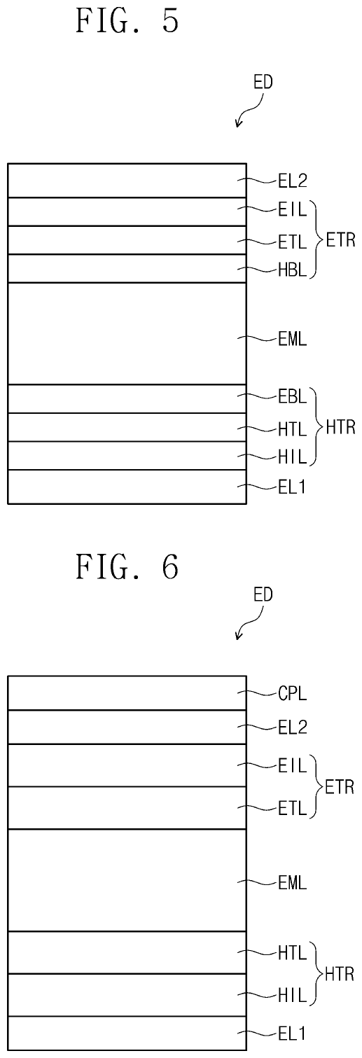

Compared with FIG. 3, FIG. 4 illustrates a cross-sectional view of a light emitting element ED, in which a hole transport region HTR includes a hole injection layer HIL and a hole transport layer HTL, and an electron transport region ETR includes an electron injection layer EIL and an electron transport layer ETL. In addition, compared with FIG. 3, FIG. 5 illustrates a cross-sectional view of a light emitting element ED, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting element ED of an embodiment including a capping layer CPL on a second electrode EL2.

The light emitting element ED may include an amine compound of an embodiment, described below, in the hole transport region HTR. In the light emitting element ED of an embodiment, at least one selected from a hole injection layer HIL, a hole transport layer HTL, and an electron blocking layer EBL in the hole transport region HTR may include the amine compound. For example, in the light emitting element ED, at least one selected from the hole transport layer HTL and the electron blocking layer EBL in the hole transport region HTR may include the amine compound.

In the light emitting element ED according to an embodiment, the first electrode EU has conductivity. The first electrode EL1 may be formed of a metal material, a metal alloy, or a conductive compound. The first electrode EU may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected from among these, a mixture of two or more selected from among these, or an oxide or oxides thereof.

When the first electrode EU is the transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EU is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or compounds or mixture or mixtures thereof (e.g., a mixture of Ag and Mg). Alternatively, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but the embodiment of the present disclosure is not limited thereto. However, the embodiment of the present disclosure is not limited thereto, and the first electrode EL1 may include the above-described metal materials, combinations of at least two metal materials of the above-described metal materials, oxides of the above-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may have a single layer formed of a single material, a single layer formed of a plurality of different materials, or a multilayer structure including a plurality of layers formed of a plurality of different materials.

The hole transport region HTR may include at least one selected from the hole injection layer HIL, the hole transport layer HTL, and the electron blocking layer EBL. In addition, the hole transport region HTR may have a stacked structure of the hole injection layer HIL and the hole transport layer HTL.

In some embodiments, the hole transport region HTR may have a single layer structure of the hole injection layer HIL or the hole transport layer HTL, or may have a single layer structure formed of a hole injection material and a hole transport material. In an embodiment, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, or a hole transport layer HTL/buffer layer are stacked in order from the first electrode EL1, but the embodiment of the present disclosure is not limited thereto.

The thickness of the hole transport region HTR may be, for example, from about 50 Å to about 15,000 Å. The hole transport region HTR may be formed using one or more suitable methods such as a vacuum deposition method, a spin coating method, a cast method, a Langmuir-Blodgett (LB) method, an inkjet printing method, a laser printing method, and a laser induced thermal imaging (LITI) method.

The light emitting element ED may include the amine compound in the hole transport region HTR. The hole transport layer HTL or the electron blocking layer EBL in the light emitting element ED may include the amine compound.

The amine compound may contain a substituent in which an aromatic hydrocarbon ring is fused to a 2-carbazole skeleton. For example, the amine compound may contain a benzocarbazole moiety. The amine compound may contain the benzocarbazole moiety in which an aromatic hydrocarbon ring is fused at the position in which HOMO orbital extends, thereby improving stability of radical or radical cation state. In some embodiments, the benzocarbazole moiety contained in the amine compound has a structure in which an aromatic hydrocarbon ring extends in the outside of the molecule, intermolecular π-π interaction may be enhanced, the hole transport property may be improved, which may contribute to reduced driving voltage and high efficiency. When the amine compound is used as a material for forming a light emitting element, to suppress an increase in deposition temperature, the benzocarbazole moiety may be a substituted or unsubstituted phenyl group that is bonded to a nitrogen atom.

The amine compound may contain a linker part of a polyphenylene group between the benzocarbazole moiety and the nitrogen atom of an amine moiety. For example, in the amine compound, the nitrogen atom of the amine moiety may be bonded to the linker part of the polyphenylene group at the para-position with respect to the benzocarbazole moiety.

In some embodiments, the amine compound may contain, in the amine moiety, at least one substituent of a substituted or unsubstituted naphthyl group, a substituted or unsubstituted phenanthryl group, a substituted or unsubstituted benzoheterole group, or a substituted fluorenyl group. The substituents may be linked to the nitrogen atom of the amine moiety with an aromatic hydrocarbon ring or an aromatic heterocycle located therebetween, or may be directly linked to the nitrogen atom of the amine moiety. At least one substituent among the substituents is introduced into the amine compound, and thus electron resistance and exciton resistance may be improved.

As described above, when the amine compound containing the benzocarbazole moiety and the foregoing substituents is used as a material for forming a light emitting element, high efficiency and long service life characteristics of the light emitting element may be achieved.

For example, the amine compound may be represented by Formula 1:

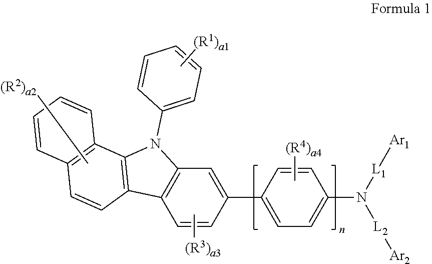

In Formula 1, R¹ to R⁴ may each independently be a hydrogen atom or a deuterium atom. For example, R¹ to R⁴ may all be hydrogen atoms, or at least one among R¹ to R⁴ may be a hydrogen atom.

In Formula 1, a1 may be an integer from 0 to 5, a2 may be an integer from 0 to 6, a3 may be an integer from 0 to 3, and a4 may be an integer from 0 to 4. Here, the embodiment in which a1 is 0 may be the same as the embodiment in which a1 is 1 and R¹ is a hydrogen atom, and the embodiment in which a2 is 0 may be the same as the embodiment in which a2 is 1 and R² is a hydrogen atom. In addition, the embodiment in which a3 is 0 may be the same as the embodiment in which a3 is 1 and R³ is a hydrogen atom, and the embodiment in which a4 is 0 may be the same as the embodiment in which a4 is 1 and R⁴ is a hydrogen atom.

In Formula 1, n may be 1 or 2. For example, n may be 1.

In Formula 1, L₁ and L₂ may be each independently a direct linkage, a substituted or unsubstituted arylene group having 6 to 30 ring-forming carbon atoms, or a substituted or unsubstituted heteroarylene group having 5 to 30 ring-forming carbon atoms. For example, L₁ and L₂ may each independently be a direct linkage or a substituted or unsubstituted arylene group having 6 to 30 ring-forming carbon atoms. For example, L₁ and L₂ may each independently be a direct linkage, a substituted or unsubstituted phenylene group, a substituted or unsubstituted biphenylene group, or a substituted or unsubstituted terphenylene group. However, the embodiment of the present disclosure is not limited thereto.

In Formula 1, Ar₁ and Ar₂ may each independently be a substituted or unsubstituted aryl group having 6 to 40 ring-forming carbon atoms, a substituted or unsubstituted heteroaryl group having 5 to 40 ring-forming carbon atoms, or any one among Formula 2 to Formula 5. In an embodiment, Ar₁ and Ar₂ may each independently be a substituted or unsubstituted aryl group having 6 to 30 ring-forming carbon atoms, or a substituted or unsubstituted heteroaryl group having 5 to 20 ring-forming carbon atoms, or one selected from among Formula 2 to Formula 5. For example, Ar₁ and Ar₂ may each independently be a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthryl group, a substituted or unsubstituted dibenzothiophene group, a substituted or unsubstituted dibenzofuran group, a substituted or unsubstituted benzonaphthothiophene group, a substituted or unsubstituted benzonaphthofuran group, or a substituted or unsubstituted fluorenyl group. In the amine compound represented by Formula 1, at least one selected from Ar₁ and Ar₂ may be one selected from among Formula 2 to Formula 5:

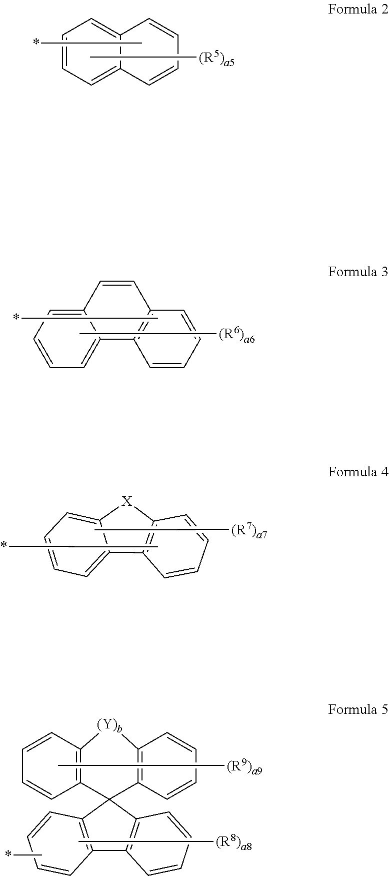

In Formulae 2 to 5, [symbol] may be a part bonded to L₁ or L₂ in Formula 1.

In Formulae 2 to 5, R⁵ to R⁹ may each be independently a hydrogen atom, a deuterium atom, a halogen atom, a substituted or unsubstituted aryl group having 6 to 30 ring-forming carbon atoms, a substituted or unsubstituted a heteroaryl group having 5 to 30 ring-forming carbon atoms, a substituted or unsubstituted alkyl group having 1 to 30 carbon atoms, or a substituted or unsubstituted alkenyl group having 2 to 30 carbon atoms, or may be bonded to an adjacent group to form a ring.

For example, in Formulae 2, 3, and 5, R⁵, R⁶, R⁸, and R⁹ may be each independently a hydrogen atom, a deuterium atom, or a substituted or unsubstituted aryl group having 6 to 30 ring-forming carbon atoms. R⁵, R⁶, R⁸, and R⁹ may each independently be a hydrogen atom, a deuterium atom, or a substituted or unsubstituted phenyl group. In some embodiments, in Formula 4, R⁷ may be a hydrogen atom, a deuterium atom, or a substituted or unsubstituted aryl group having 6 to 30 ring-forming carbon atoms, or may be bonded to an adjacent group to form a ring. R⁷ may be a hydrogen atom, a deuterium atom, or a substituted or unsubstituted phenyl group, or may be bonded to an adjacent group to form a ring of a dibenzoheterole derivative.

In Formulae 2 to 5, a5, a7, and a8 may each independently be an integer from 0 to 7, a6 may be an integer from 0 to 9, and a9 may be an integer from 0 to 8. Here, the embodiment in which a5 is 0 may be the same as the embodiment in which a5 is 1 and R⁵ is a hydrogen atom. The embodiment in which a6 is 0 may be the same as the embodiment in which a6 is 1 and R⁶ is a hydrogen atom. The embodiment in which a7 is 0 may be the same as the embodiment in which a7 is 1 and R⁷ is a hydrogen atom. In addition, the embodiment in which a8 is 0 may be the same as the embodiment in which a8 is 1 and R⁸ is a hydrogen atom, and the case where a9 is 0 may be the same as the embodiment in which a9 is 1 and R⁹ is a hydrogen atom.

When each of a5 to a9 is an integer of 2 or more, a plurality of R⁵s to R⁹s may each be the same or different. For example, when a5 is 2, two R⁵s may be the same as or different from each other. In addition, such a description may be equally applied to R⁶ to R⁹.

In some embodiments, in Formula 4, X may be O or S. For example, when X is O, Formula 4 may be a dibenzofuran group substituted or unsubstituted with at least one R⁷. When X is S, Formula 4 may be a dibenzothiophene group substituted or unsubstituted with at least one R⁷.

In Formula 5, b may be 0 or 1. For example, when b is 0, Formula 5 may be represented by Formula 5-A. When b is 1, Y may be a direct linkage, and may be represented by Formula 5-B:

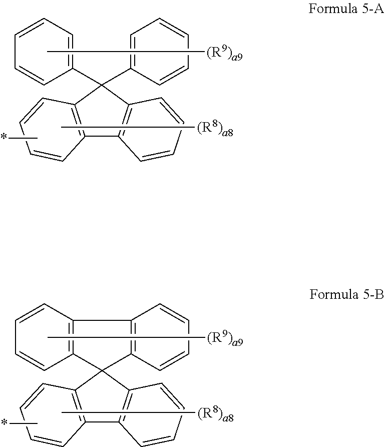

In Formula 5-A and Formula 5-B, the same as those described in Formula 5 may be applied to R⁸ and R⁹, and a8 and a9.

For example, for the amine compound, in Formula 1, when one selected from among Ar₁ and Ar₂ is Formula 2, the other may be Formula 2 or Formula 3. When one selected from among Ar₁ and Ar₂ is Formula 3, the other may be a substituted or unsubstituted aryl group having 6 to 40 ring-forming carbon atoms, a substituted or unsubstituted heteroaryl group having 5 to 40 ring-forming carbon atoms, or one selected from among Formula 3 to Formula 5. When one selected from among Ar₁ and Ar₂ above is Formula 4, the other may be a substituted or unsubstituted aryl group having 6 to 40 ring-forming carbon atoms, or a substituted or unsubstituted heteroaryl group having 5 to 40 ring-forming carbon atoms, or one selected from among Formula 4 and Formula 5. When one selected from among An and Ar₂ is Formula 5, the other may be a substituted or unsubstituted aryl group having 6 to 40 ring-forming carbon atoms, or a substituted or unsubstituted heteroaryl group having 5 to 40 ring-forming carbon atoms. However, the embodiment of the present disclosure is not limited thereto.

In an embodiment, Formula 1 may be represented by Formula 1-1. Formula 1-1 corresponds to the embodiment in which n is 1 in Formula 1. In Formula 1-1, the same as those described in Formula 1 may be applied to R¹ to R³, a1 to a3, L₂, L₂, Ar₁, and Ar₂.

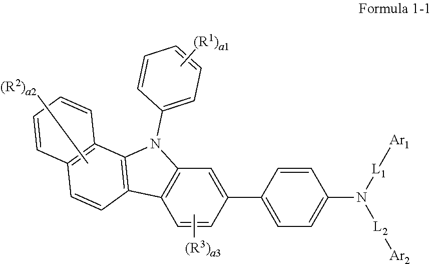

In an embodiment, Formula 2 may be represented by one selected from among 2-1 and 2-2:

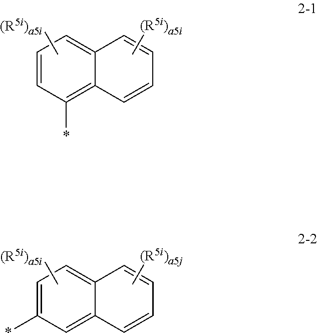

In 2-1 and 2-2, [symbol] corresponds to a part bonded to L₁ or L₂. In 2-1 and 2-2, R⁵ⁱ and R^{5j} may be each independently a hydrogen atom, a deuterium atom, or a substituted or unsubstituted aryl group having 6 to 30 ring-forming carbon atoms. For example, R⁵ⁱ and R^{5j} may each independently be a hydrogen atom, a deuterium atom, or a substituted or unsubstituted phenyl group. However, the embodiment of the present disclosure is not limited thereto.

In 2-1 and 2-2, a5i may be an integer from 0 to 3, and a5j may be an integer from 0 to 4. Here, the embodiment in which a5i is 0 may be the same as the embodiment in which a5i is 1 and R⁵ⁱ is a hydrogen atom, and the embodiment in which a5j is 0 may be the same as the embodiment in which a5j is 1 and R^{5j} is a hydrogen atom. When each of a5i to a5j is an integer of 2 or more, a plurality of R⁵ⁱ's and R^{5j}'s may each be the same or different.

In an embodiment, Formula 3 may be represented by one selected from among 3-1 to 3-3:

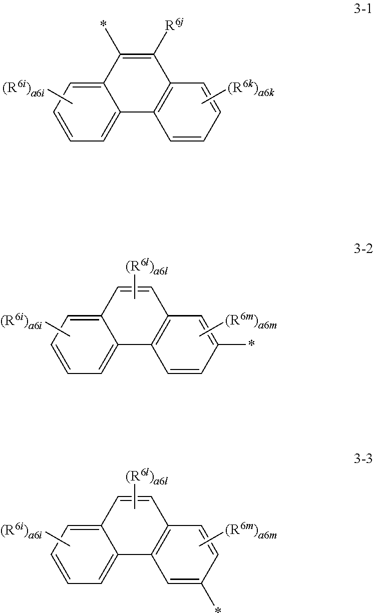

In 3-1 to 3-3, [symbol] corresponds to a part bonded to L₁ or L₂. In 3-1 to 3-3, R⁶ⁱ, R^{6j}, R^{6k}, R^{6l}, and R^{6m} may each independently be a hydrogen atom, a deuterium atom, or a substituted or unsubstituted aryl group having 6 to 30 ring-forming carbon atoms. For example, R⁶ⁱ, R^{6j}, R^{6k}, R^{6l} and R^{6m} may each independently be a hydrogen atom, a deuterium atom, or a substituted or unsubstituted phenyl group. However, the embodiment of the present disclosure is not limited thereto.

In 3-1 to 3-3, a6i may be an integer from 0 to 4. In 3-1, a6k may be an integer from 0 to 4, in 3-2 and 3-3, a61 may be an integer from 0 to 2, and in 3-2 and 3-3, a6m may be an integer from 0 to 3. Here, the embodiment in which a6i is 0 may be the same as the embodiment in which a6i is 1 and R⁶ⁱ is a hydrogen atom. Such a description may be equally applied to embodiments in which a6k, a6l, and a6m are 0. When each of a6i, a6k, a6l and a6m is an integer from 2 or more, a plurality of R⁶ⁱs, R^{6k}s, R^{6l}s and R^{6m}s may each be the same or different.

In an embodiment, Formula 4 may be represented by one selected from among 4-1 to 4-4.

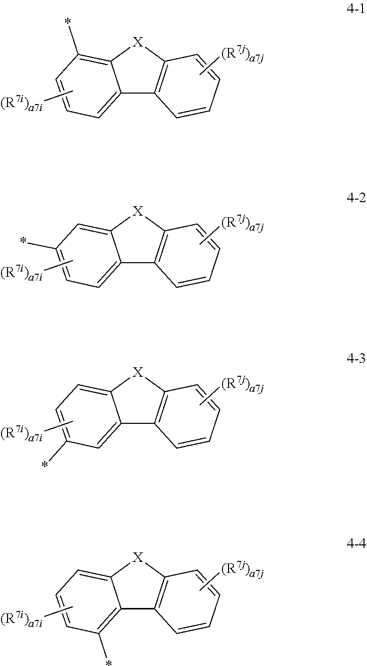

In 4-1 to 4-4, [symbol] corresponds to a part bonded to L₁ or L₂. In 4-1 to 4-4, R⁷ⁱ and R^{7j} may each independently be a hydrogen atom, a deuterium atom, or a substituted or unsubstituted aryl group having 6 to 30 ring-forming carbon atoms, or may be bonded to an adjacent group to form a ring. R⁷ⁱ and R^{7j} may each independently be a hydrogen atom, a deuterium atom, or a substituted or unsubstituted phenyl group, or may be bonded to an adjacent group to form a ring of a dibenzoheterole derivative. For example, when R⁷ⁱ or R^{7j} is bonded to an adjacent group to form a ring of a dibenzoheterole derivative, 4-1 to 4-4 may include a skeleton of

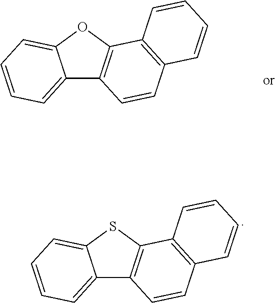 [SMILES: c1ccc2c(c1)ccc1c3ccccc3oc21 ; c1ccc2c(c1)ccc1c3ccccc3sc21]

However, the embodiment of the present disclosure is not limited thereto.

In 4-1 to 4-4, a7i may be an integer of 0 to 3, and a7j may be an integer of 0 to 4. Here, the embodiment in which a7i is 0 may be the same as the embodiment in which a7i is 1 and R⁷ⁱ is a hydrogen atom, and the embodiment in which a7j is 0 may be the same as the embodiment in which a7j is 1 and R^{7j} is a hydrogen atom. When each of a7i to a7j is an integer of 2 or more, a plurality of R⁷ⁱ's and R^{7j}'s may each be the same or different.

In an embodiment, Formula 5 may be represented by one selected from among 5-1 to 5-6:

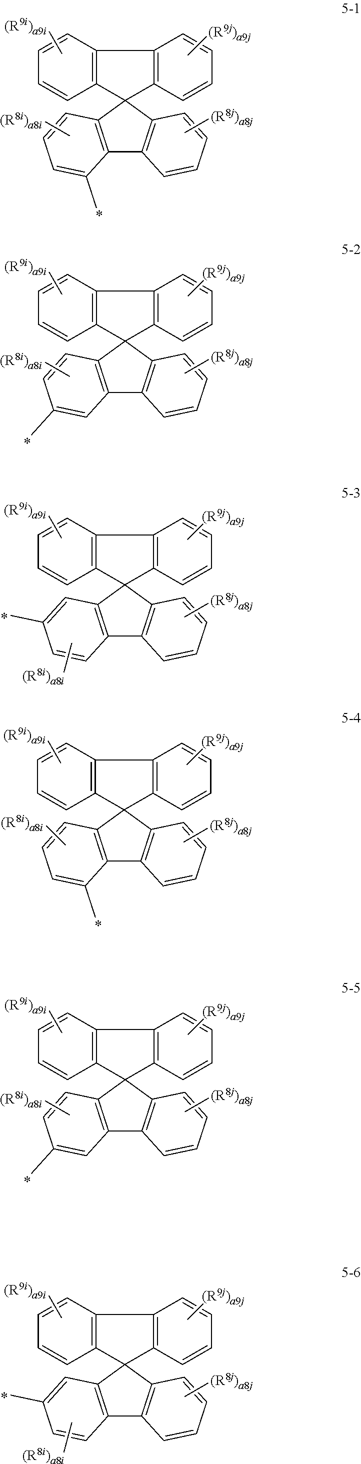

In 5-1 to 5-6, [symbol] corresponds to a part bonded to L₁ or L₂. In 5-1 to 5-6, R⁸ⁱ, R^{8j}, R⁹ⁱ, and R^{9j} may each independently be a hydrogen atom, a deuterium atom, or a substituted or unsubstituted aryl group having 6 to 30 ring-forming carbon atoms, or may be bonded to an adjacent group to form a ring. For example, R⁸ⁱ, R^{8j}, R⁹ⁱ, and R^{9j} may each independently be a hydrogen atom, a deuterium atom, or a substituted or unsubstituted phenyl group. However, the embodiment of the present disclosure is not limited thereto.

In 5-1 to 5-6, a8i may be an integer of 0 to 3, and a8j, a9i and a9j may each independently be an integer of 0 to 4. Here, the embodiment in which a8i is 0 may be the same as the embodiment in which a8i is 1 and R⁸ⁱ is a hydrogen atom. Such a description may be equally applied to the embodiment in which a8j, a9i, and a9j are 0. When each of a8i, a8j, a9i and a9j is an integer of 2 or more, a plurality of R⁸ⁱ's, R^{8j}'s, R⁹ⁱ's and R^{9j}'s may each be the same or different.

The amine compound represented by Formula 1 may be represented by one selected from among the compounds of Compound Group 1. The hole transport region HTR of the light emitting element ED may include at least one selected from among the amine compounds disclosed in Compound Group 1. D in Compound Group 1 is a deuterium atom.

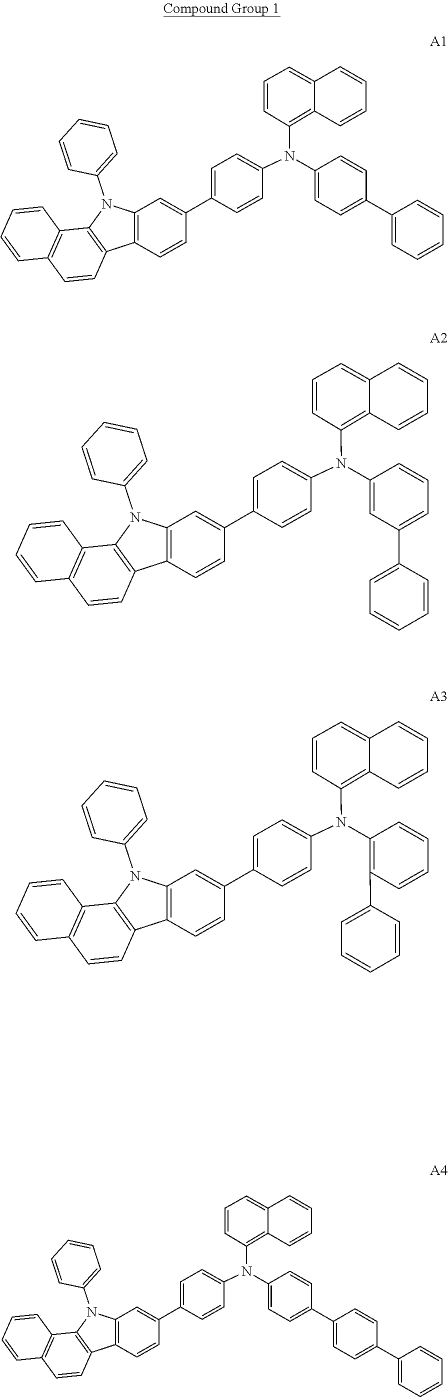 [SMILES: c1ccc(-c2ccc(N(c3ccc(-c4ccc5c6ccc7ccccc7c6n(-c6ccccc6)c5c4)cc3)c3cccc4ccccc34)cc2)cc1 ; c1ccc(-c2cccc(N(c3ccc(-c4ccc5c6ccc7ccccc7c6n(-c6ccccc6)c5c4)cc3)c3cccc4ccccc34)c2)cc1 ; c1ccc(-c2ccccc2N(c2ccc(-c3ccc4c5ccc6ccccc6c5n(-c5ccccc5)c4c3)cc2)c2cccc3ccccc23)cc1 ; c1ccc(-c2ccc(-c3ccc(N(c4ccc(-c5ccc6c7ccc8ccccc8c7n(-c7ccccc7)c6c5)cc4)c4cccc5ccccc45)cc3)cc2)cc1]

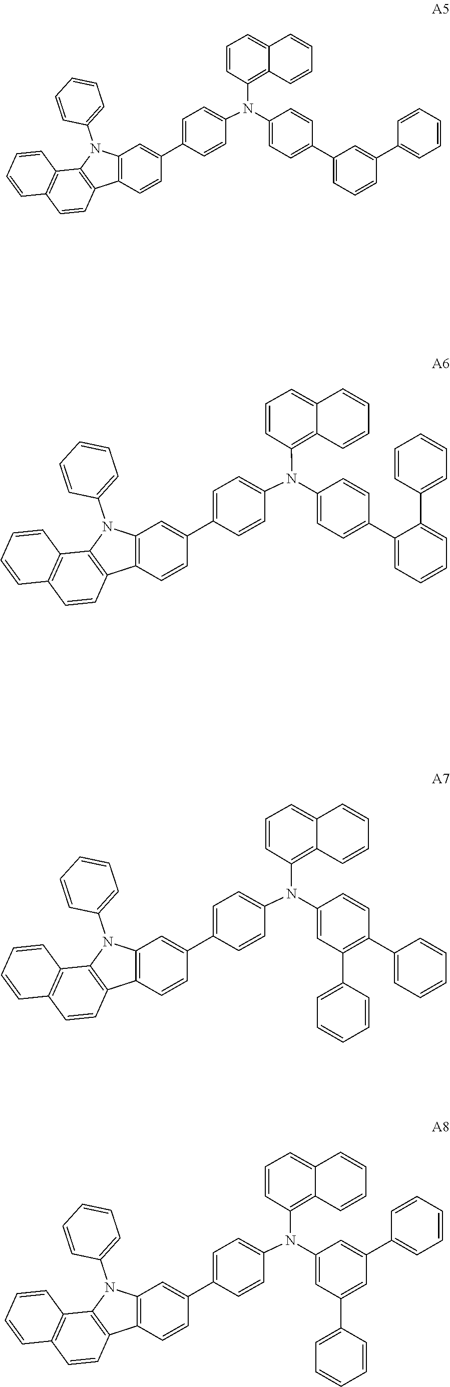 [SMILES: c1ccc(-c2cccc(-c3ccc(N(c4ccc(-c5ccc6c7ccc8ccccc8c7n(-c7ccccc7)c6c5)cc4)c4cccc5ccccc45)cc3)c2)cc1 ; c1ccc(-c2ccccc2-c2ccc(N(c3ccc(-c4ccc5c6ccc7ccccc7c6n(-c6ccccc6)c5c4)cc3)c3cccc4ccccc34)cc2)cc1 ; c1ccc(-c2ccc(N(c3ccc(-c4ccc5c6ccc7ccccc7c6n(-c6ccccc6)c5c4)cc3)c3cccc4ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4ccccc34)c2)cc1]

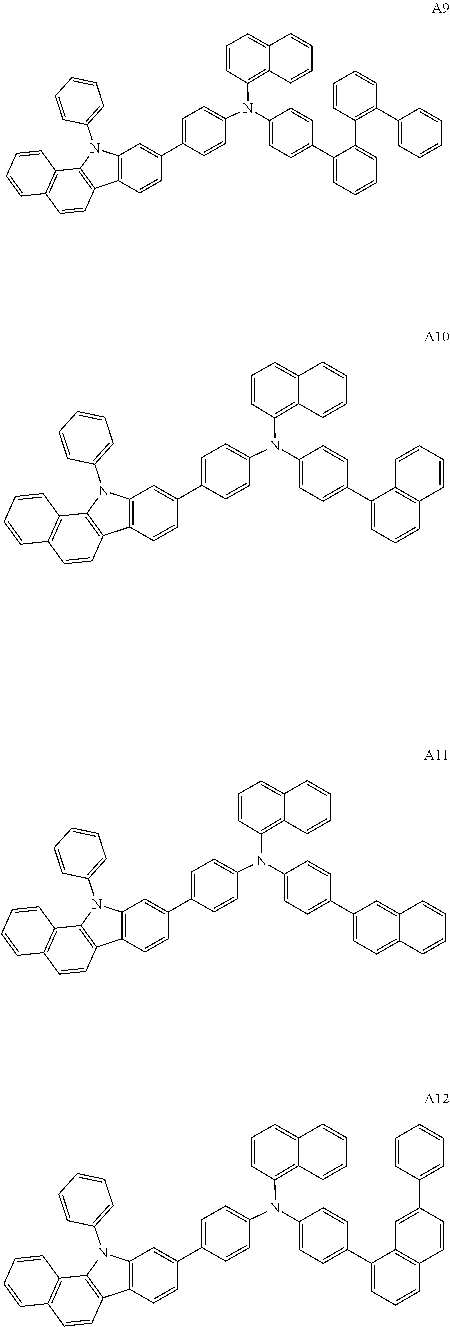 [SMILES: c1ccc(-c2ccccc2-c2ccccc2-c2ccc(N(c3ccc(-c4ccc5c6ccc7ccccc7c6n(-c6ccccc6)c5c4)cc3)c3cccc4ccccc34)cc2)cc1 ; c1ccc(-n2c3cc(-c4ccc(N(c5ccc(-c6cccc7ccccc67)cc5)c5cccc6ccccc56)cc4)ccc3c3ccc4ccccc4c32)cc1 ; c1ccc(-n2c3cc(-c4ccc(N(c5ccc(-c6ccc7ccccc7c6)cc5)c5cccc6ccccc56)cc4)ccc3c3ccc4ccccc4c32)cc1 ; c1ccc(-c2ccc3cccc(-c4ccc(N(c5ccc(-c6ccc7c8ccc9ccccc9c8n(-c8ccccc8)c7c6)cc5)c5cccc6ccccc56)cc4)c3c2)cc1]

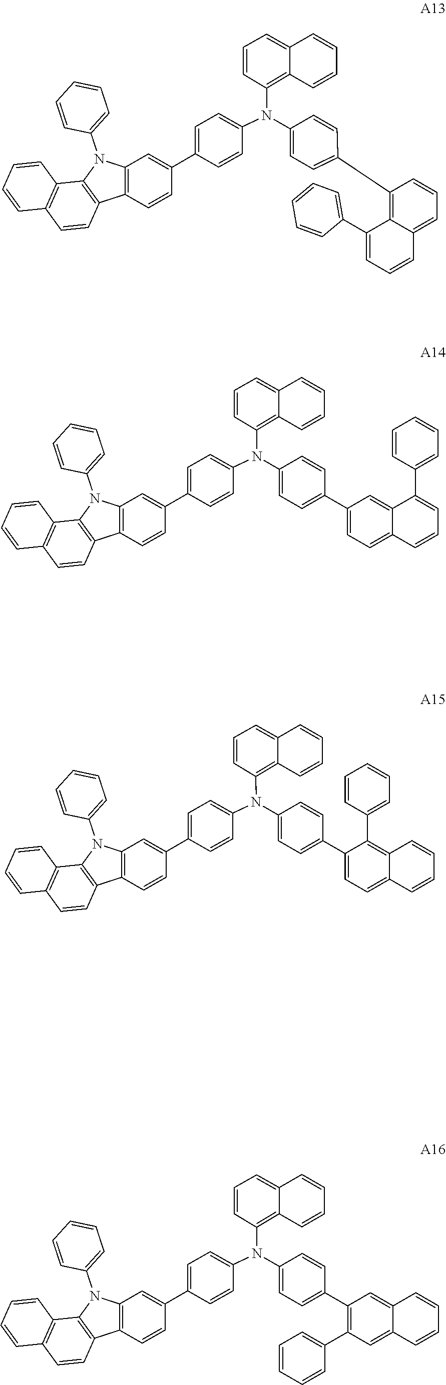 [SMILES: c1ccc(-c2cccc3cccc(-c4ccc(N(c5ccc(-c6ccc7c8ccc9ccccc9c8n(-c8ccccc8)c7c6)cc5)c5cccc6ccccc56)cc4)c23)cc1 ; c1ccc(-c2cccc3ccc(-c4ccc(N(c5ccc(-c6ccc7c8ccc9ccccc9c8n(-c8ccccc8)c7c6)cc5)c5cccc6ccccc56)cc4)cc23)cc1 ; c1ccc(-c2c(-c3ccc(N(c4ccc(-c5ccc6c7ccc8ccccc8c7n(-c7ccccc7)c6c5)cc4)c4cccc5ccccc45)cc3)ccc3ccccc23)cc1 ; c1ccc(-c2cc3ccccc3cc2-c2ccc(N(c3ccc(-c4ccc5c6ccc7ccccc7c6n(-c6ccccc6)c5c4)cc3)c3cccc4ccccc34)cc2)cc1]

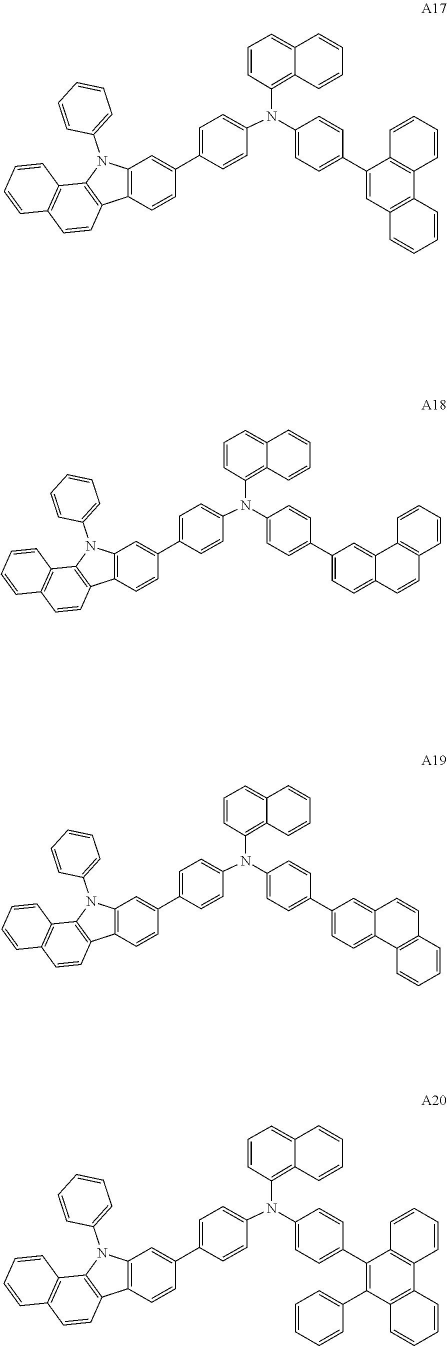 [SMILES: c1ccc(-n2c3cc(-c4ccc(N(c5ccc(-c6cc7ccccc7c7ccccc67)cc5)c5cccc6ccccc56)cc4)ccc3c3ccc4ccccc4c32)cc1 ; c1ccc(-n2c3cc(-c4ccc(N(c5ccc(-c6ccc7ccc8ccccc8c7c6)cc5)c5cccc6ccccc56)cc4)ccc3c3ccc4ccccc4c32)cc1 ; c1ccc(-n2c3cc(-c4ccc(N(c5ccc(-c6ccc7c(ccc8ccccc87)c6)cc5)c5cccc6ccccc56)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4cccc5ccccc45)cc3)c3ccccc3c3ccccc23)cc1]

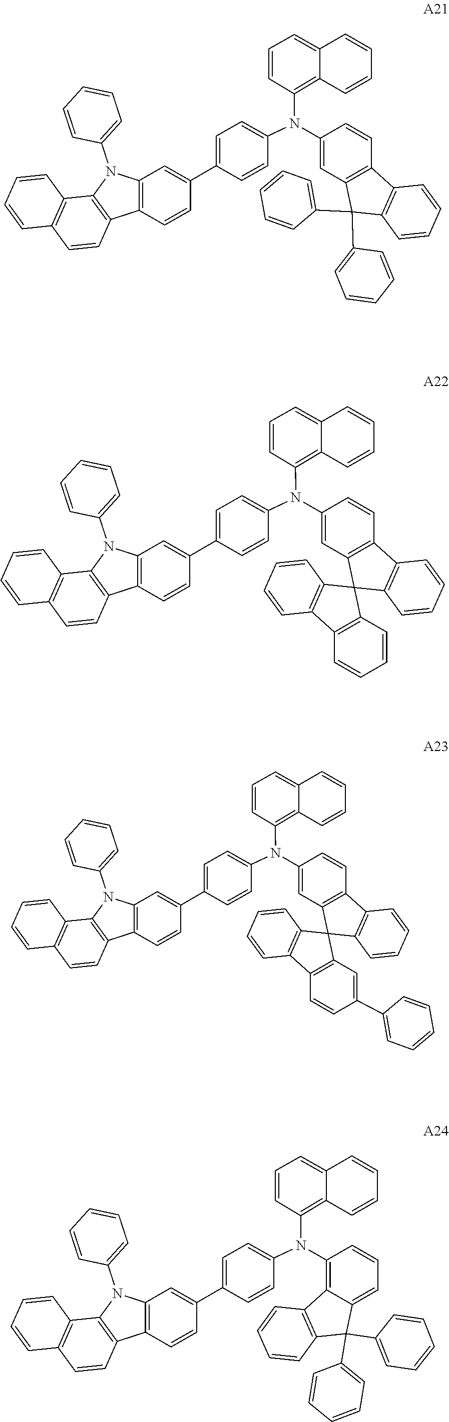 [SMILES: c1ccc(-n2c3cc(-c4ccc(N(c5ccc6c(c5)C(c5ccccc5)(c5ccccc5)c5ccccc5-6)c5cccc6ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5cccc6ccccc56)cc4)ccc3c3ccc4ccccc4c32)cc1 ; c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4cccc5ccccc45)cc32)cc1 ; c1ccc(-n2c3cc(-c4ccc(N(c5cccc6c5-c5ccccc5C6(c5ccccc5)c5ccccc5)c5cccc6ccccc56)cc4)ccc3c3ccc4ccccc4c32)cc1]

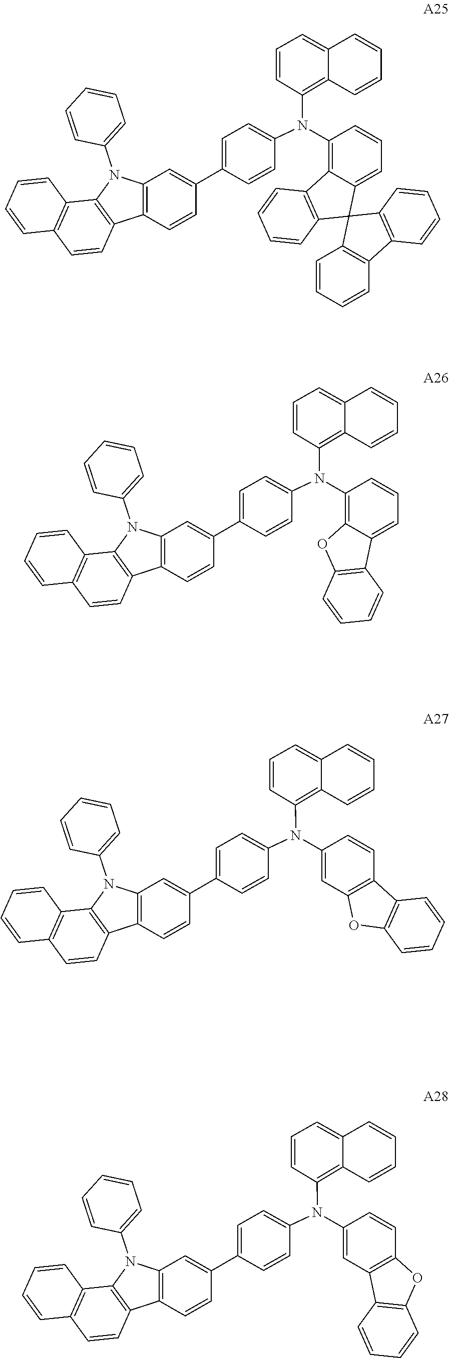 [SMILES: c1ccc(-n2c3cc(-c4ccc(N(c5cccc6c5-c5ccccc5C65c6ccccc6-c6ccccc65)c5cccc6ccccc56)cc4)ccc3c3ccc4ccccc4c32)cc1 ; c1ccc(-n2c3cc(-c4ccc(N(c5cccc6ccccc56)c5cccc6c5oc5ccccc56)cc4)ccc3c3ccc4ccccc4c32)cc1 ; c1ccc(-n2c3cc(-c4ccc(N(c5ccc6c(c5)oc5ccccc56)c5cccc6ccccc56)cc4)ccc3c3ccc4ccccc4c32)cc1 ; c1ccc(-n2c3cc(-c4ccc(N(c5ccc6oc7ccccc7c6c5)c5cccc6ccccc56)cc4)ccc3c3ccc4ccccc4c32)cc1]

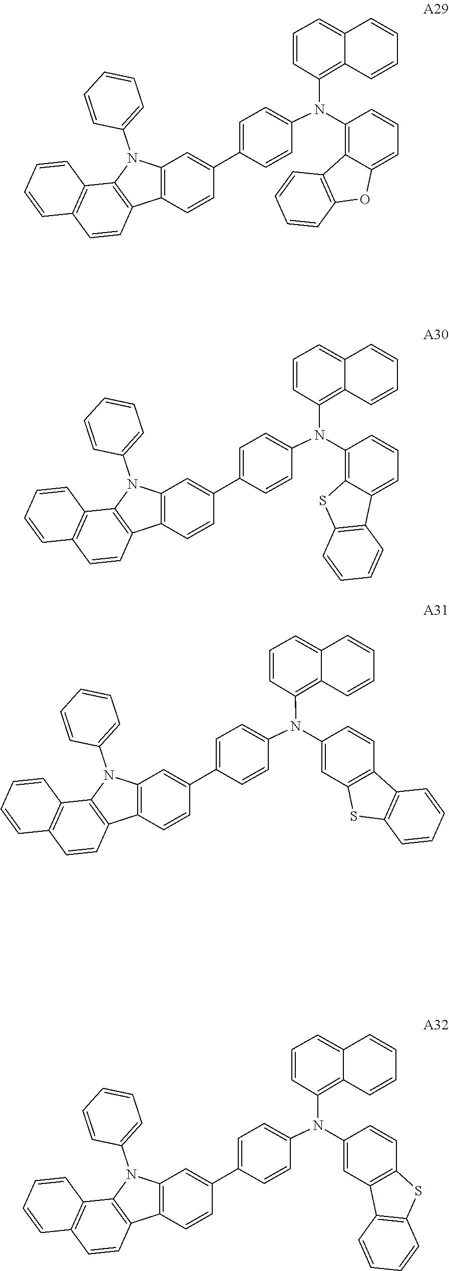 [SMILES: c1ccc(-n2c3cc(-c4ccc(N(c5cccc6ccccc56)c5cccc6oc7ccccc7c56)cc4)ccc3c3ccc4ccccc4c32)cc1 ; c1ccc(-n2c3cc(-c4ccc(N(c5cccc6ccccc56)c5cccc6c5sc5ccccc56)cc4)ccc3c3ccc4ccccc4c32)cc1 ; c1ccc(-n2c3cc(-c4ccc(N(c5ccc6c(c5)sc5ccccc56)c5cccc6ccccc56)cc4)ccc3c3ccc4ccccc4c32)cc1 ; c1ccc(-n2c3cc(-c4ccc(N(c5ccc6sc7ccccc7c6c5)c5cccc6ccccc56)cc4)ccc3c3ccc4ccccc4c32)cc1]

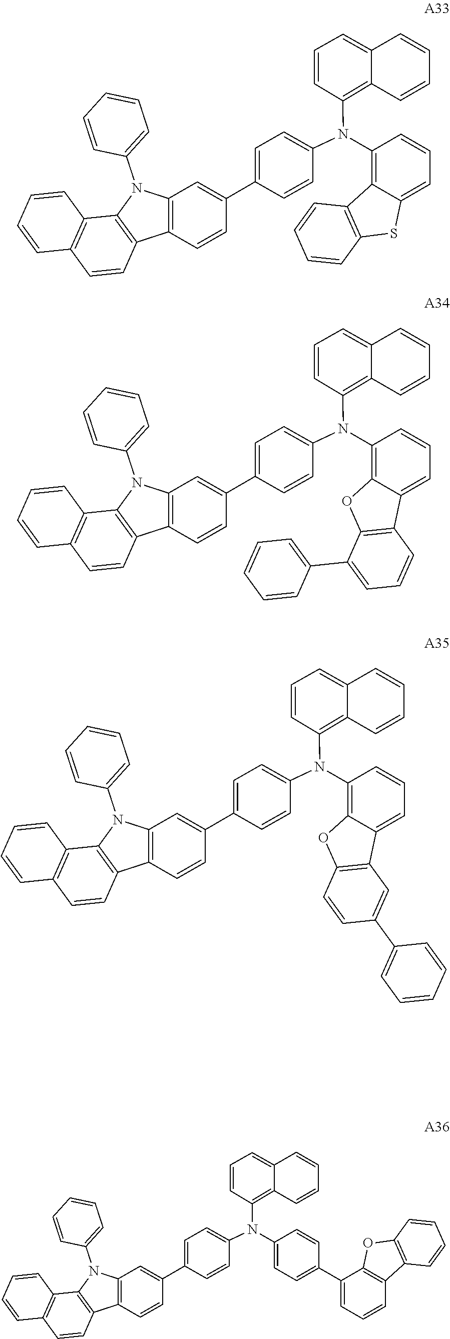 [SMILES: c1ccc(-n2c3cc(-c4ccc(N(c5cccc6ccccc56)c5cccc6sc7ccccc7c56)cc4)ccc3c3ccc4ccccc4c32)cc1 ; c1ccc(-c2cccc3c2oc2c(N(c4ccc(-c5ccc6c7ccc8ccccc8c7n(-c7ccccc7)c6c5)cc4)c4cccc5ccccc45)cccc23)cc1 ; c1ccc(-c2ccc3oc4c(N(c5ccc(-c6ccc7c8ccc9ccccc9c8n(-c8ccccc8)c7c6)cc5)c5cccc6ccccc56)cccc4c3c2)cc1 ; c1ccc(-n2c3cc(-c4ccc(N(c5ccc(-c6cccc7c6oc6ccccc67)cc5)c5cccc6ccccc56)cc4)ccc3c3ccc4ccccc4c32)cc1]

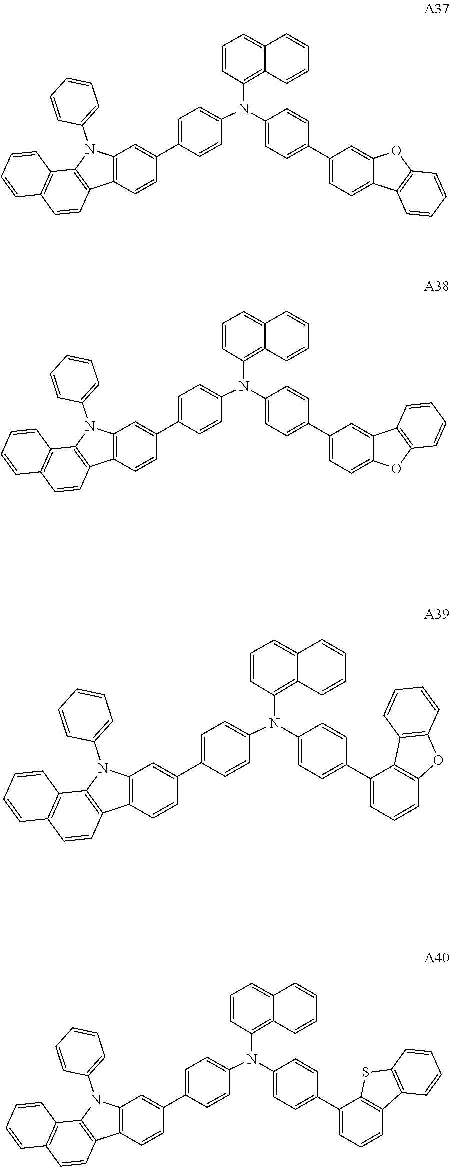 [SMILES: c1ccc(-n2c3cc(-c4ccc(N(c5ccc(-c6ccc7c(c6)oc6ccccc67)cc5)c5cccc6ccccc56)cc4)ccc3c3ccc4ccccc4c32)cc1 ; c1ccc(-n2c3cc(-c4ccc(N(c5ccc(-c6ccc7oc8ccccc8c7c6)cc5)c5cccc6ccccc56)cc4)ccc3c3ccc4ccccc4c32)cc1 ; c1ccc(-n2c3cc(-c4ccc(N(c5ccc(-c6cccc7oc8ccccc8c67)cc5)c5cccc6ccccc56)cc4)ccc3c3ccc4ccccc4c32)cc1 ; c1ccc(-n2c3cc(-c4ccc(N(c5ccc(-c6cccc7c6sc6ccccc67)cc5)c5cccc6ccccc56)cc4)ccc3c3ccc4ccccc4c32)cc1]

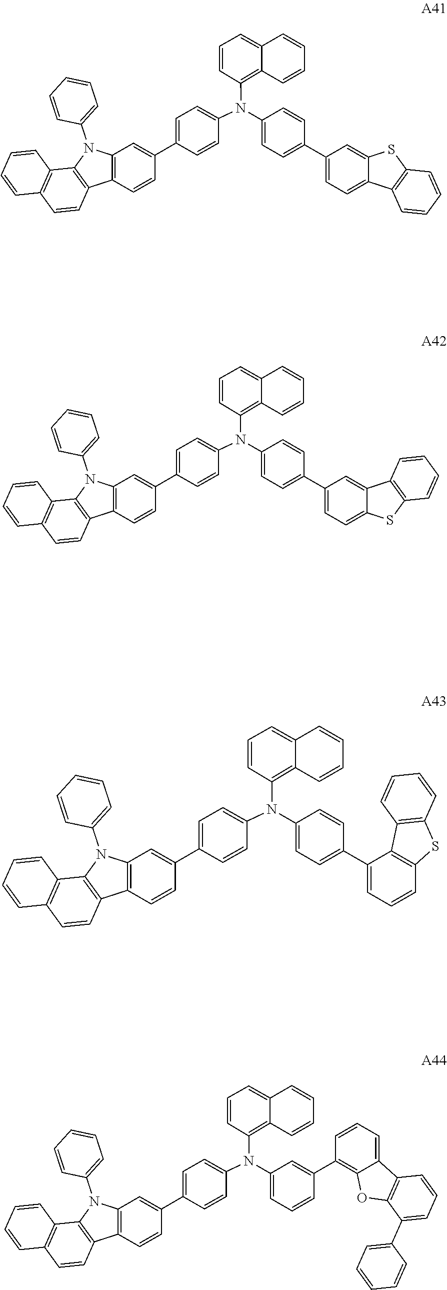 [SMILES: c1ccc(-n2c3cc(-c4ccc(N(c5ccc(-c6ccc7c(c6)sc6ccccc67)cc5)c5cccc6ccccc56)cc4)ccc3c3ccc4ccccc4c32)cc1 ; c1ccc(-n2c3cc(-c4ccc(N(c5ccc(-c6ccc7sc8ccccc8c7c6)cc5)c5cccc6ccccc56)cc4)ccc3c3ccc4ccccc4c32)cc1 ; c1ccc(-n2c3cc(-c4ccc(N(c5ccc(-c6cccc7sc8ccccc8c67)cc5)c5cccc6ccccc56)cc4)ccc3c3ccc4ccccc4c32)cc1 ; c1ccc(-c2cccc3c2oc2c(-c4cccc(N(c5ccc(-c6ccc7c8ccc9ccccc9c8n(-c8ccccc8)c7c6)cc5)c5cccc6ccccc56)c4)cccc23)cc1]

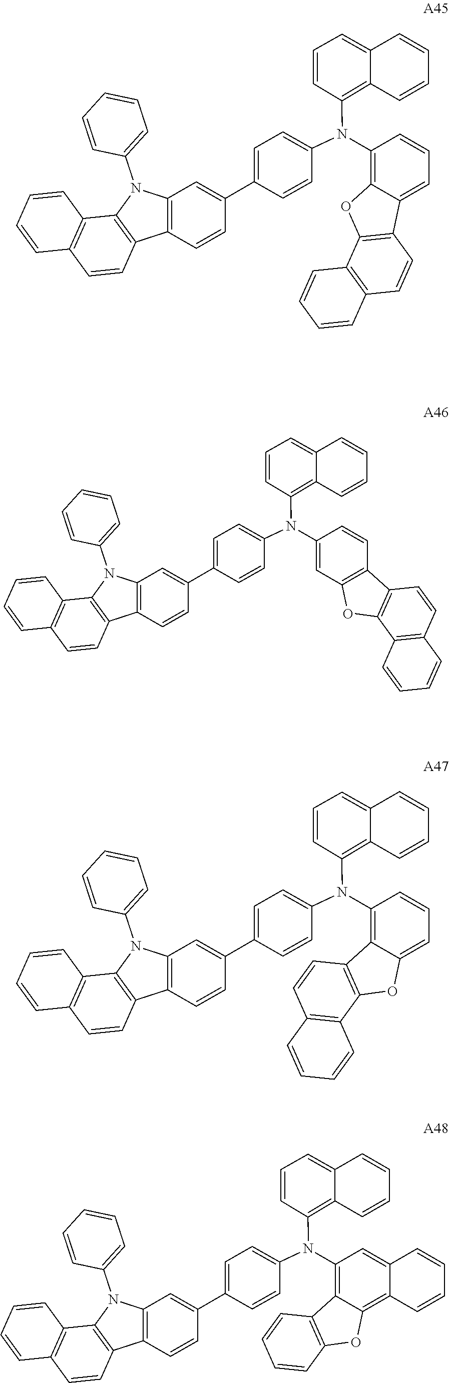 [SMILES: c1ccc(-n2c3cc(-c4ccc(N(c5cccc6ccccc56)c5cccc6c5oc5c7ccccc7ccc65)cc4)ccc3c3ccc4ccccc4c32)cc1 ; c1ccc(-n2c3cc(-c4ccc(N(c5ccc6c(c5)oc5c7ccccc7ccc65)c5cccc6ccccc56)cc4)ccc3c3ccc4ccccc4c32)cc1 ; c1ccc(-n2c3cc(-c4ccc(N(c5cccc6ccccc56)c5cccc6oc7c8ccccc8ccc7c56)cc4)ccc3c3ccc4ccccc4c32)cc1 ; c1ccc(-n2c3cc(-c4ccc(N(c5cccc6ccccc56)c5cc6ccccc6c6oc7ccccc7c56)cc4)ccc3c3ccc4ccccc4c32)cc1]

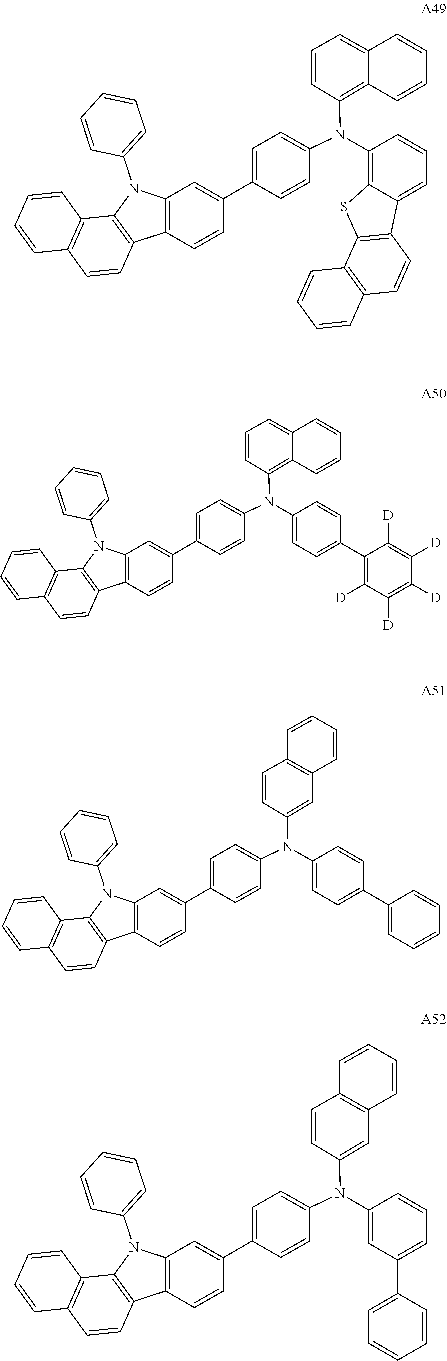 [SMILES: c1ccc(-n2c3cc(-c4ccc(N(c5cccc6ccccc56)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3cccc4ccccc34)cc2)c([2H])c1[2H] ; c1ccc(-c2ccc(N(c3ccc(-c4ccc5c6ccc7ccccc7c6n(-c6ccccc6)c5c4)cc3)c3ccc4ccccc4c3)cc2)cc1 ; c1ccc(-c2cccc(N(c3ccc(-c4ccc5c6ccc7ccccc7c6n(-c6ccccc6)c5c4)cc3)c3ccc4ccccc4c3)c2)cc1]

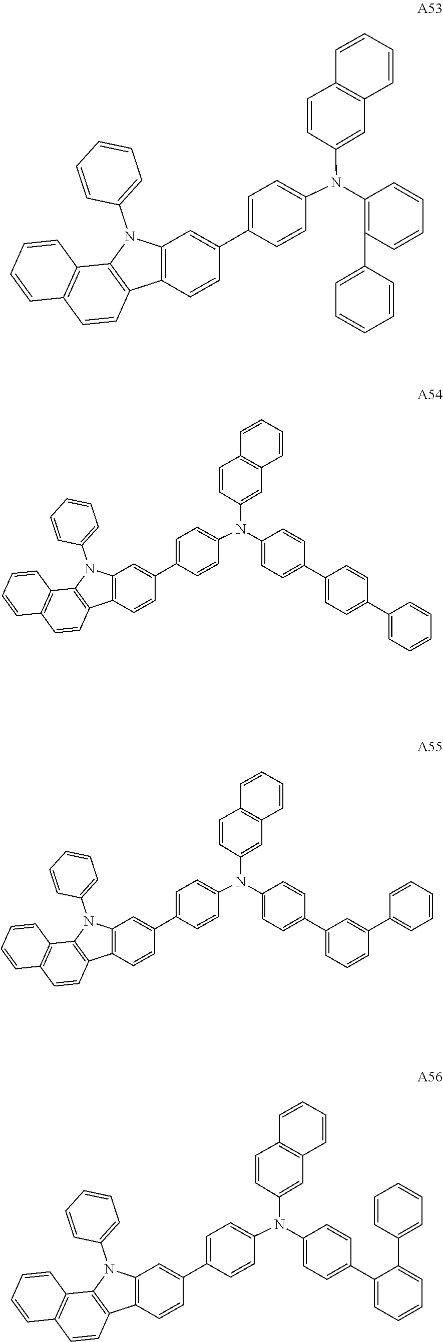 [SMILES: c1ccc(-c2ccccc2N(c2ccc(-c3ccc4c5ccc6ccccc6c5n(-c5ccccc5)c4c3)cc2)c2ccc3ccccc3c2)cc1 ; c1ccc(-c2ccc(-c3ccc(N(c4ccc(-c5ccc6c7ccc8ccccc8c7n(-c7ccccc7)c6c5)cc4)c4ccc5ccccc5c4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5ccccc5c4)cc3)c2)cc1 ; c1ccc(-c2ccccc2-c2ccc(N(c3ccc(-c4ccc5c6ccc7ccccc7c6n(-c6ccccc6)c5c4)cc3)c3ccc4ccccc4c3)cc2)cc1]

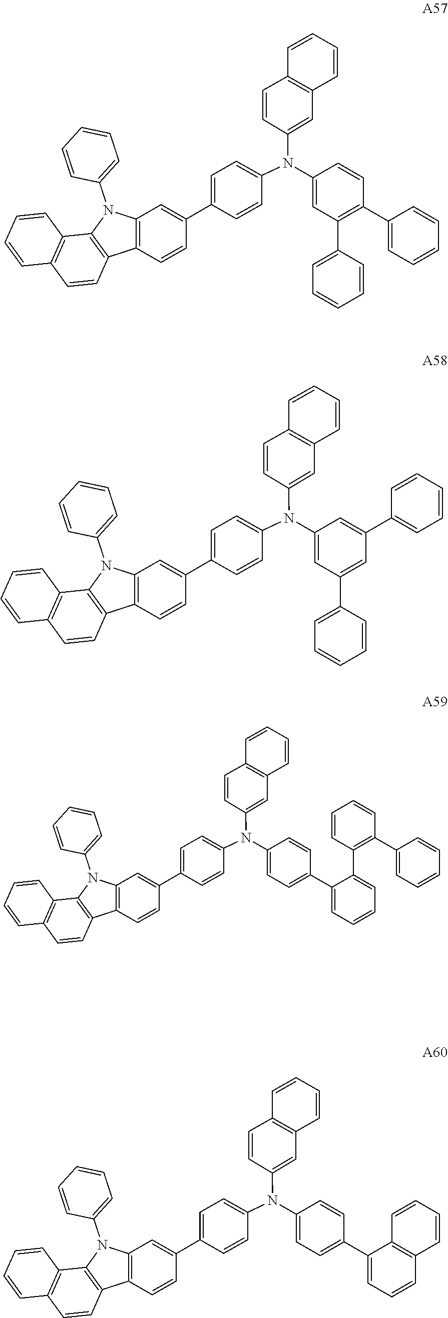 [SMILES: c1ccc(-c2ccc(N(c3ccc(-c4ccc5c6ccc7ccccc7c6n(-c6ccccc6)c5c4)cc3)c3ccc4ccccc4c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4ccccc4c3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4ccccc4c3)cc2)cc1 ; c1ccc(-n2c3cc(-c4ccc(N(c5ccc(-c6cccc7ccccc67)cc5)c5ccc6ccccc6c5)cc4)ccc3c3ccc4ccccc4c32)cc1]

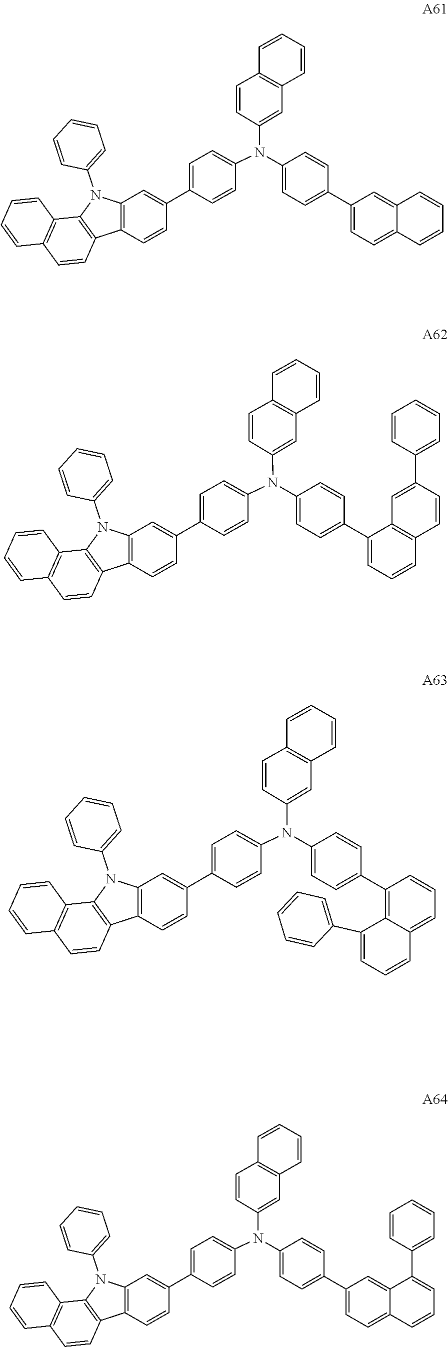 [SMILES: c1ccc(-n2c3cc(-c4ccc(N(c5ccc(-c6ccc7ccccc7c6)cc5)c5ccc6ccccc6c5)cc4)ccc3c3ccc4ccccc4c32)cc1 ; c1ccc(-c2ccc3cccc(-c4ccc(N(c5ccc(-c6ccc7c8ccc9ccccc9c8n(-c8ccccc8)c7c6)cc5)c5ccc6ccccc6c5)cc4)c3c2)cc1 ; c1ccc(-c2cccc3cccc(-c4ccc(N(c5ccc(-c6ccc7c8ccc9ccccc9c8n(-c8ccccc8)c7c6)cc5)c5ccc6ccccc6c5)cc4)c23)cc1 ; c1ccc(-c2cccc3ccc(-c4ccc(N(c5ccc(-c6ccc7c8ccc9ccccc9c8n(-c8ccccc8)c7c6)cc5)c5ccc6ccccc6c5)cc4)cc23)cc1]

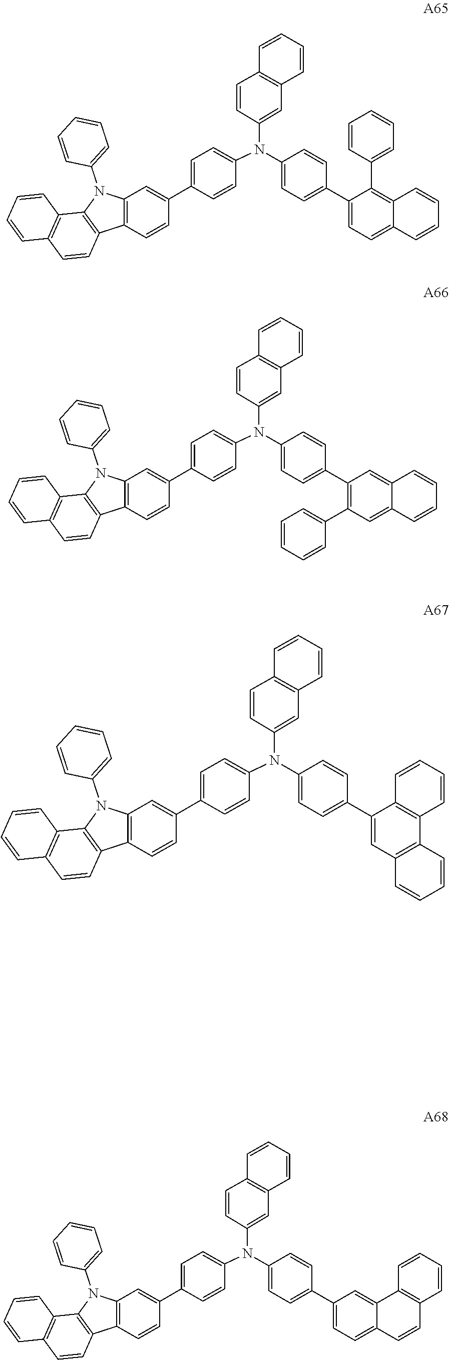 [SMILES: c1ccc(-c2c(-c3ccc(N(c4ccc(-c5ccc6c7ccc8ccccc8c7n(-c7ccccc7)c6c5)cc4)c4ccc5ccccc5c4)cc3)ccc3ccccc23)cc1 ; c1ccc(-c2cc3ccccc3cc2-c2ccc(N(c3ccc(-c4ccc5c6ccc7ccccc7c6n(-c6ccccc6)c5c4)cc3)c3ccc4ccccc4c3)cc2)cc1 ; c1ccc(-n2c3cc(-c4ccc(N(c5ccc(-c6cc7ccccc7c7ccccc67)cc5)c5ccc6ccccc6c5)cc4)ccc3c3ccc4ccccc4c32)cc1 ; c1ccc(-n2c3cc(-c4ccc(N(c5ccc(-c6ccc7ccc8ccccc8c7c6)cc5)c5ccc6ccccc6c5)cc4)ccc3c3ccc4ccccc4c32)cc1]

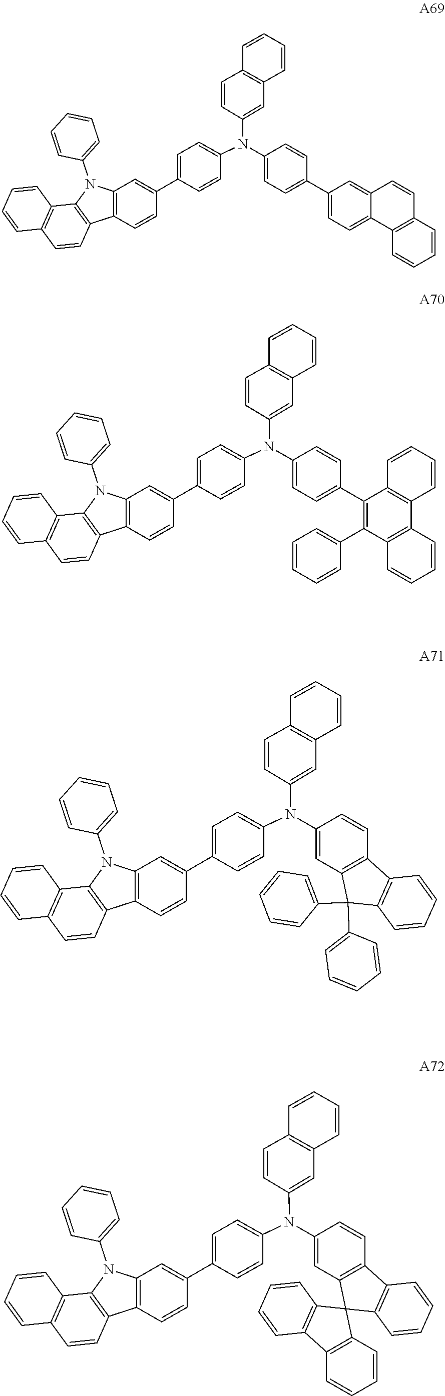 [SMILES: c1ccc(-n2c3cc(-c4ccc(N(c5ccc(-c6ccc7c(ccc8ccccc87)c6)cc5)c5ccc6ccccc6c5)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4ccc5ccccc5c4)cc3)c3ccccc3c3ccccc23)cc1 ; c1ccc(-n2c3cc(-c4ccc(N(c5ccc6c(c5)C(c5ccccc5)(c5ccccc5)c5ccccc5-6)c5ccc6ccccc6c5)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6ccccc6c5)cc4)ccc3c3ccc4ccccc4c32)cc1]

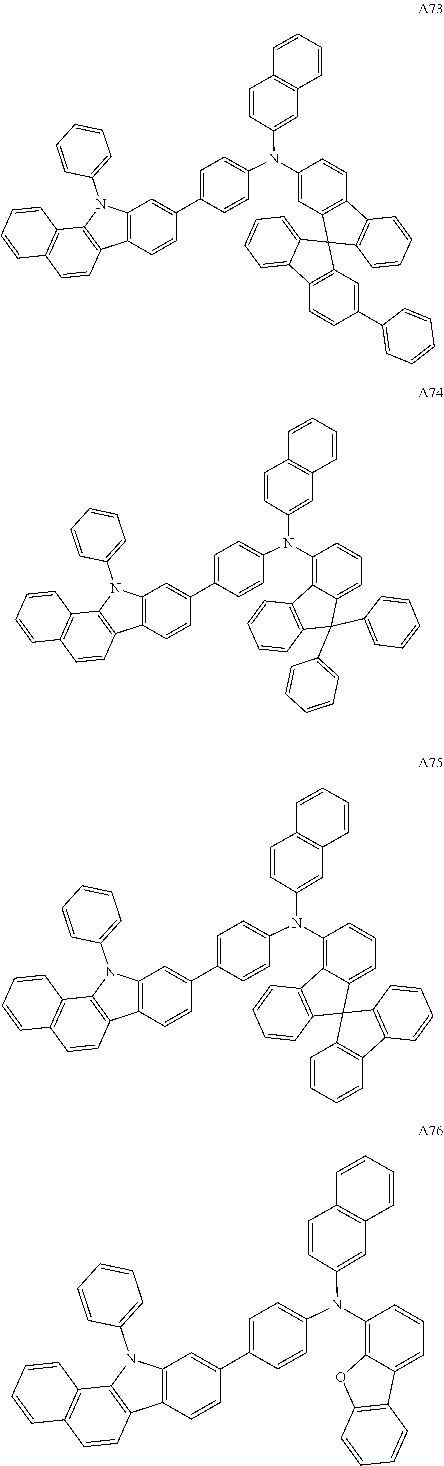 [SMILES: c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4ccc5ccccc5c4)cc32)cc1 ; c1ccc(-n2c3cc(-c4ccc(N(c5ccc6ccccc6c5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc6ccccc6c5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc6ccccc6c5)c5cccc6c5oc5ccccc56)cc4)ccc3c3ccc4ccccc4c32)cc1]

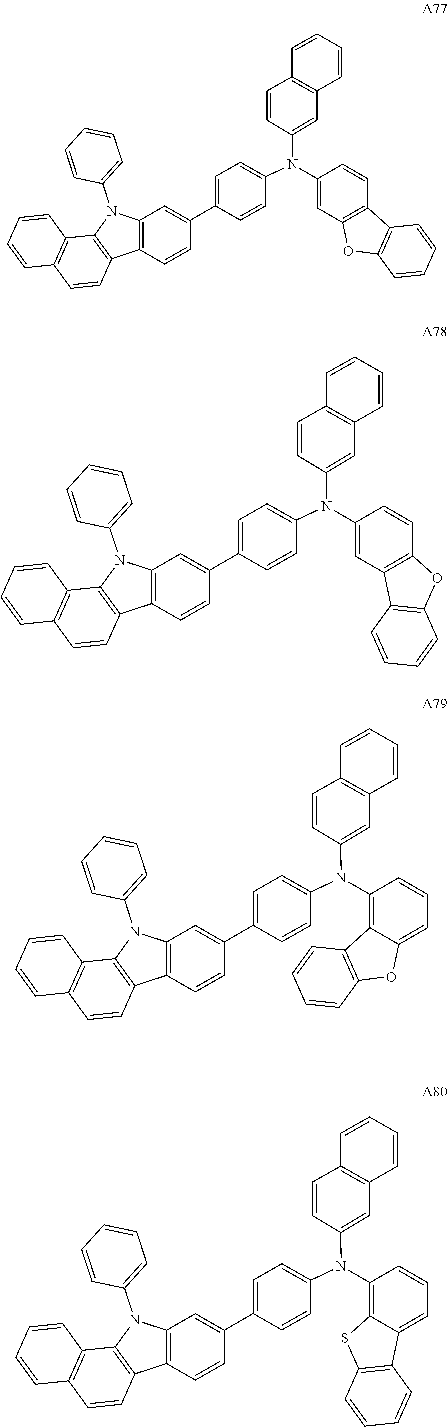 [SMILES: c1ccc(-n2c3cc(-c4ccc(N(c5ccc6ccccc6c5)c5ccc6c(c5)oc5ccccc56)cc4)ccc3c3ccc4ccccc4c32)cc1 ; c1ccc(-n2c3cc(-c4ccc(N(c5ccc6ccccc6c5)c5ccc6oc7ccccc7c6c5)cc4)ccc3c3ccc4ccccc4c32)cc1 ; c1ccc(-n2c3cc(-c4ccc(N(c5ccc6ccccc6c5)c5cccc6oc7ccccc7c56)cc4)ccc3c3ccc4ccccc4c32)cc1 ; c1ccc(-n2c3cc(-c4ccc(N(c5ccc6ccccc6c5)c5cccc6c5sc5ccccc56)cc4)ccc3c3ccc4ccccc4c32)cc1]

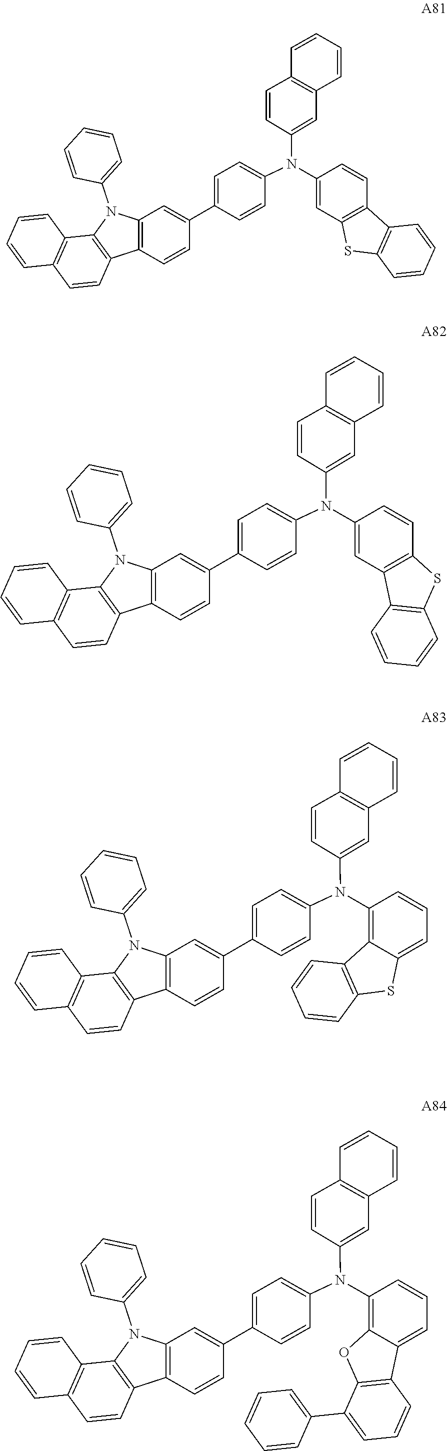 [SMILES: c1ccc(-n2c3cc(-c4ccc(N(c5ccc6ccccc6c5)c5ccc6c(c5)sc5ccccc56)cc4)ccc3c3ccc4ccccc4c32)cc1 ; c1ccc(-n2c3cc(-c4ccc(N(c5ccc6ccccc6c5)c5ccc6sc7ccccc7c6c5)cc4)ccc3c3ccc4ccccc4c32)cc1 ; c1ccc(-n2c3cc(-c4ccc(N(c5ccc6ccccc6c5)c5cccc6sc7ccccc7c56)cc4)ccc3c3ccc4ccccc4c32)cc1 ; c1ccc(-c2cccc3c2oc2c(N(c4ccc(-c5ccc6c7ccc8ccccc8c7n(-c7ccccc7)c6c5)cc4)c4ccc5ccccc5c4)cccc23)cc1]

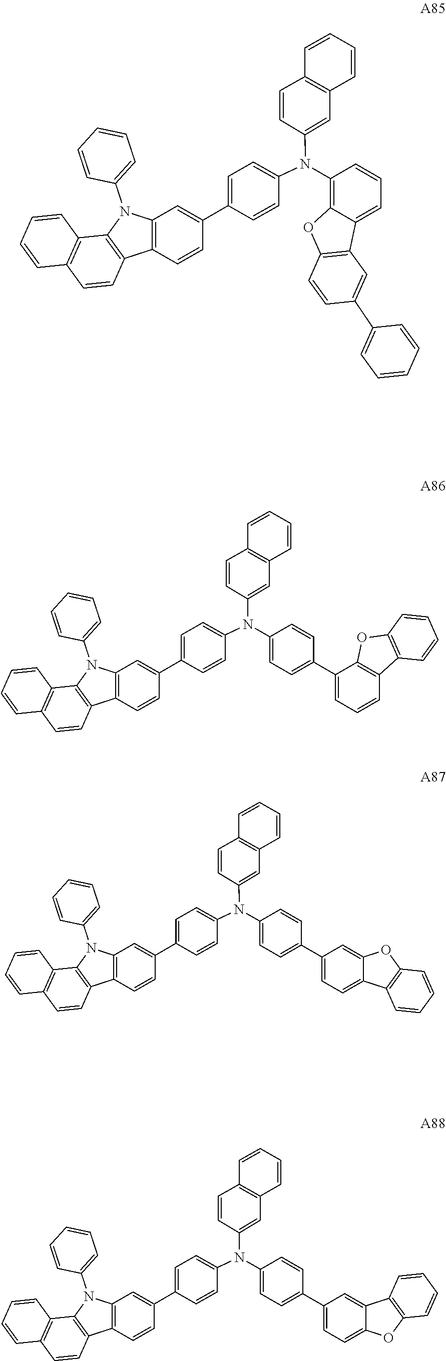 [SMILES: c1ccc(-c2ccc3oc4c(N(c5ccc(-c6ccc7c8ccc9ccccc9c8n(-c8ccccc8)c7c6)cc5)c5ccc6ccccc6c5)cccc4c3c2)cc1 ; c1ccc(-n2c3cc(-c4ccc(N(c5ccc(-c6cccc7c6oc6ccccc67)cc5)c5ccc6ccccc6c5)cc4)ccc3c3ccc4ccccc4c32)cc1 ; c1ccc(-n2c3cc(-c4ccc(N(c5ccc(-c6ccc7c(c6)oc6ccccc67)cc5)c5ccc6ccccc6c5)cc4)ccc3c3ccc4ccccc4c32)cc1 ; c1ccc(-n2c3cc(-c4ccc(N(c5ccc(-c6ccc7oc8ccccc8c7c6)cc5)c5ccc6ccccc6c5)cc4)ccc3c3ccc4ccccc4c32)cc1]

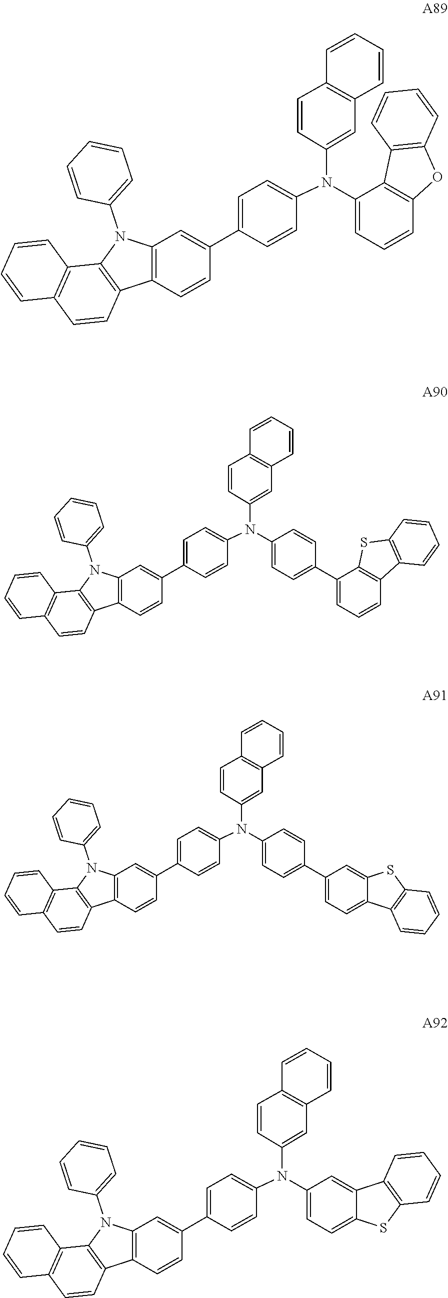 [SMILES: c1ccc(-n2c3cc(-c4ccc(N(c5ccc6ccccc6c5)c5cccc6oc7ccccc7c56)cc4)ccc3c3ccc4ccccc4c32)cc1 ; c1ccc(-n2c3cc(-c4ccc(N(c5ccc(-c6cccc7c6sc6ccccc67)cc5)c5ccc6ccccc6c5)cc4)ccc3c3ccc4ccccc4c32)cc1 ; c1ccc(-n2c3cc(-c4ccc(N(c5ccc(-c6ccc7c(c6)sc6ccccc67)cc5)c5ccc6ccccc6c5)cc4)ccc3c3ccc4ccccc4c32)cc1 ; c1ccc(-n2c3cc(-c4ccc(N(c5ccc6ccccc6c5)c5ccc6sc7ccccc7c6c5)cc4)ccc3c3ccc4ccccc4c32)cc1]

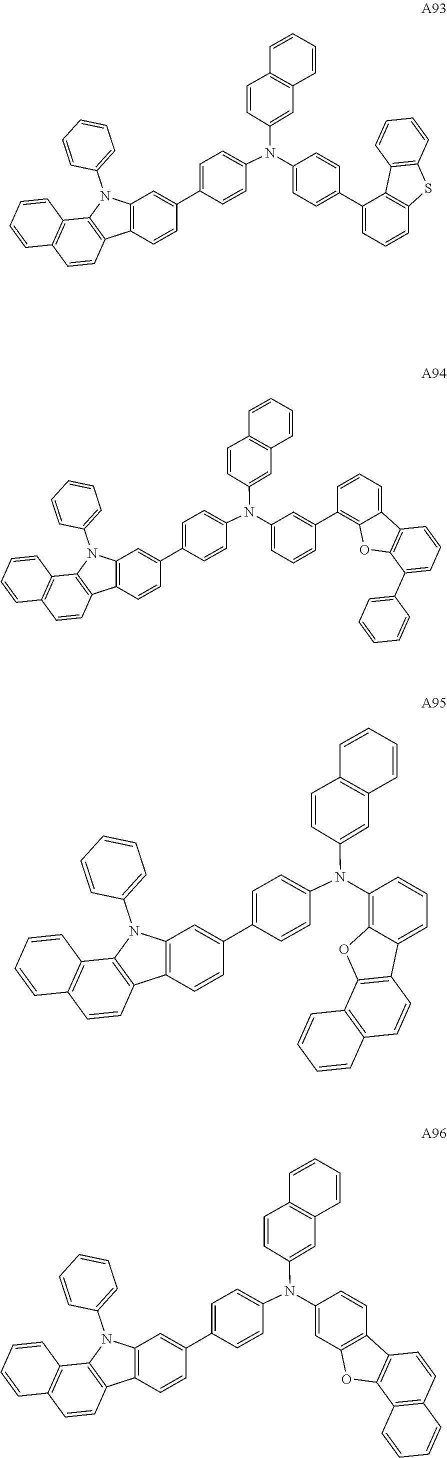 [SMILES: c1ccc(-n2c3cc(-c4ccc(N(c5ccc(-c6cccc7sc8ccccc8c67)cc5)c5ccc6ccccc6c5)cc4)ccc3c3ccc4ccccc4c32)cc1 ; c1ccc(-c2cccc3c2oc2c(-c4cccc(N(c5ccc(-c6ccc7c8ccc9ccccc9c8n(-c8ccccc8)c7c6)cc5)c5ccc6ccccc6c5)c4)cccc23)cc1 ; c1ccc(-n2c3cc(-c4ccc(N(c5ccc6ccccc6c5)c5cccc6c5oc5c7ccccc7ccc65)cc4)ccc3c3ccc4ccccc4c32)cc1 ; c1ccc(-n2c3cc(-c4ccc(N(c5ccc6ccccc6c5)c5ccc6c(c5)oc5c7ccccc7ccc65)cc4)ccc3c3ccc4ccccc4c32)cc1]

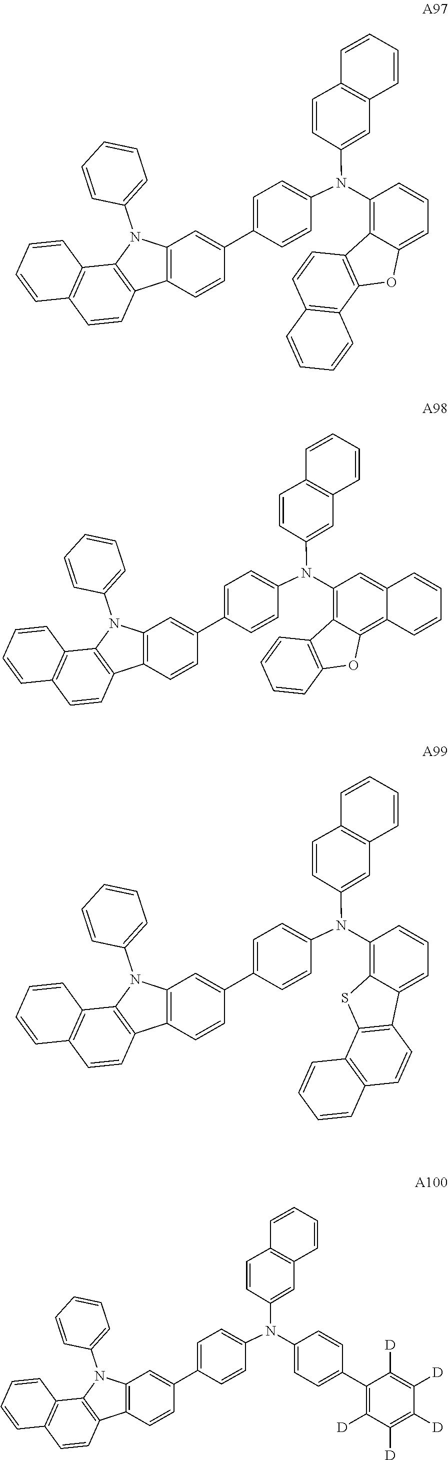 [SMILES: c1ccc(-n2c3cc(-c4ccc(N(c5ccc6ccccc6c5)c5cccc6oc7c8ccccc8ccc7c56)cc4)ccc3c3ccc4ccccc4c32)cc1 ; c1ccc(-n2c3cc(-c4ccc(N(c5ccc6ccccc6c5)c5cc6ccccc6c6oc7ccccc7c56)cc4)ccc3c3ccc4ccccc4c32)cc1 ; c1ccc(-n2c3cc(-c4ccc(N(c5ccc6ccccc6c5)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4ccccc4c3)cc2)c([2H])c1[2H]]

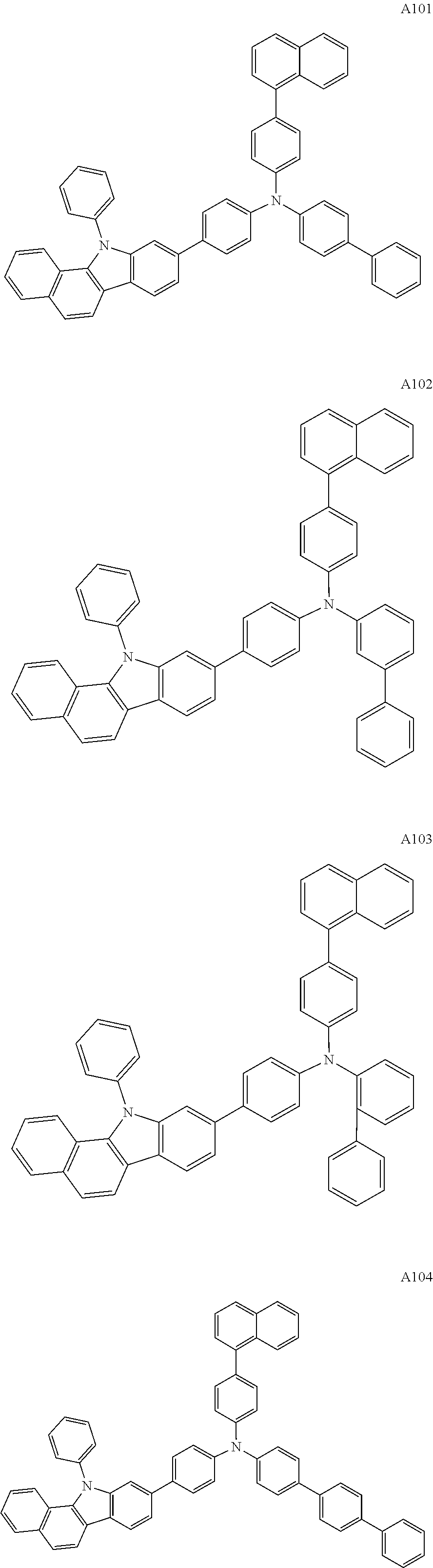 [SMILES: c1ccc(-c2ccc(N(c3ccc(-c4ccc5c6ccc7ccccc7c6n(-c6ccccc6)c5c4)cc3)c3ccc(-c4cccc5ccccc45)cc3)cc2)cc1 ; c1ccc(-c2cccc(N(c3ccc(-c4ccc5c6ccc7ccccc7c6n(-c6ccccc6)c5c4)cc3)c3ccc(-c4cccc5ccccc45)cc3)c2)cc1 ; c1ccc(-c2ccccc2N(c2ccc(-c3ccc4c5ccc6ccccc6c5n(-c5ccccc5)c4c3)cc2)c2ccc(-c3cccc4ccccc34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6ccccc56)cc4)cc3)cc2)cc1]

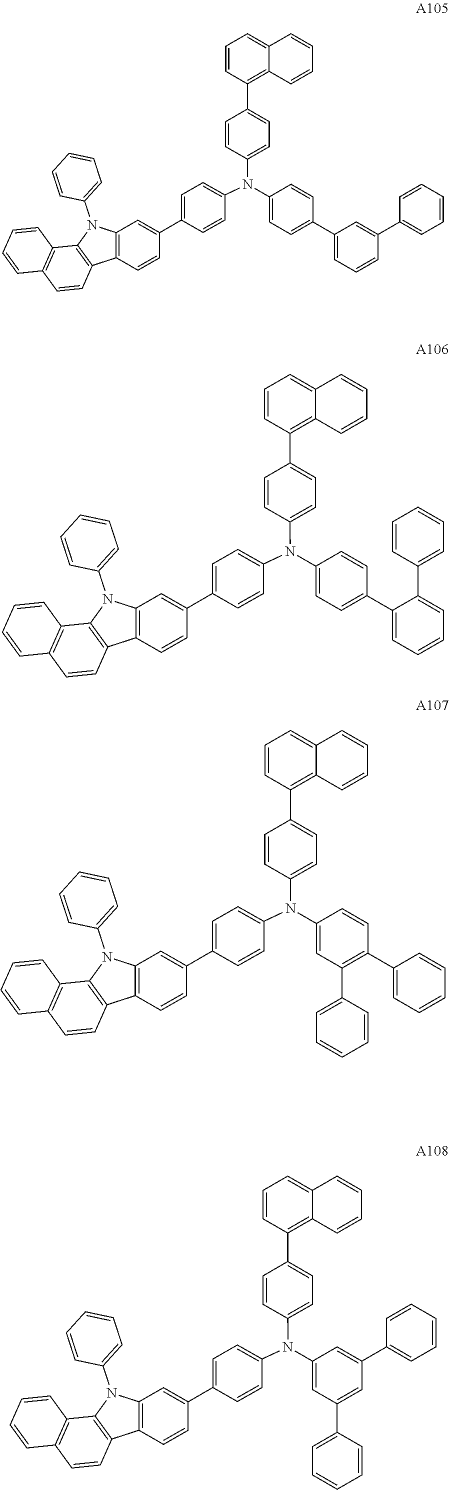 [SMILES: c1ccc(-c2cccc(-c3ccc(N(c4ccc(-c5ccc6c7ccc8ccccc8c7n(-c7ccccc7)c6c5)cc4)c4ccc(-c5cccc6ccccc56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5ccccc45)cc3)cc2)cc1 ; c1ccc(-c2ccc(N(c3ccc(-c4ccc5c6ccc7ccccc7c6n(-c6ccccc6)c5c4)cc3)c3ccc(-c4cccc5ccccc45)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cccc5ccccc45)cc3)c2)cc1]

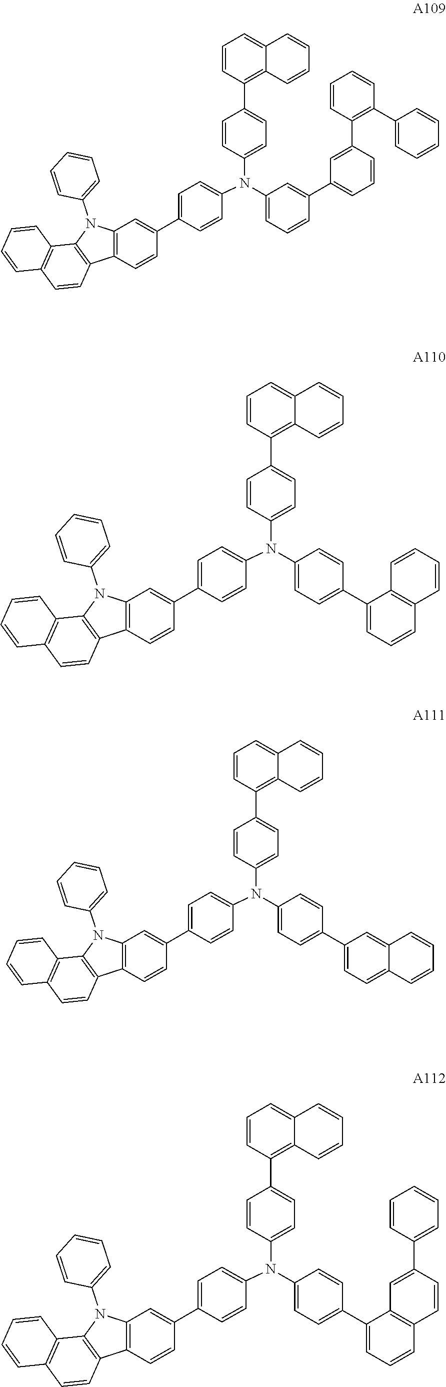 [SMILES: c1ccc(-c2ccccc2-c2cccc(-c3cccc(N(c4ccc(-c5ccc6c7ccc8ccccc8c7n(-c7ccccc7)c6c5)cc4)c4ccc(-c5cccc6ccccc56)cc4)c3)c2)cc1 ; c1ccc(-n2c3cc(-c4ccc(N(c5ccc(-c6cccc7ccccc67)cc5)c5ccc(-c6cccc7ccccc67)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6cccc7ccccc67)cc5)cc4)ccc3c3ccc4ccccc4c32)cc1 ; c1ccc(-c2ccc3cccc(-c4ccc(N(c5ccc(-c6ccc7c8ccc9ccccc9c8n(-c8ccccc8)c7c6)cc5)c5ccc(-c6cccc7ccccc67)cc5)cc4)c3c2)cc1]

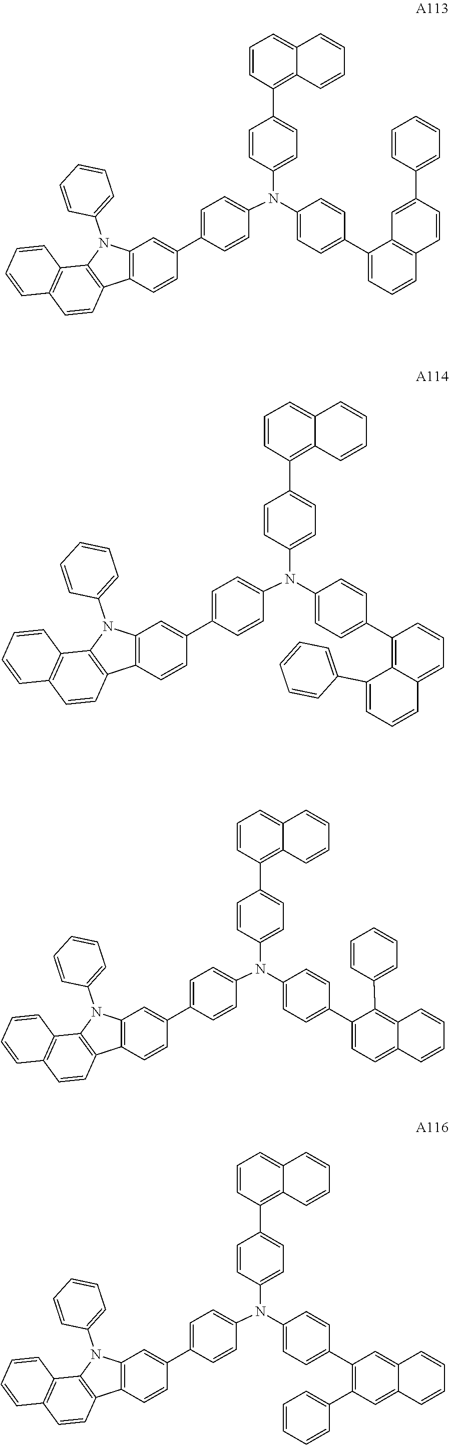 [SMILES: c1ccc(-c2ccc3cccc(-c4ccc(N(c5ccc(-c6ccc7c8ccc9ccccc9c8n(-c8ccccc8)c7c6)cc5)c5ccc(-c6cccc7ccccc67)cc5)cc4)c3c2)cc1 ; c1ccc(-c2cccc3cccc(-c4ccc(N(c5ccc(-c6ccc7c8ccc9ccccc9c8n(-c8ccccc8)c7c6)cc5)c5ccc(-c6cccc7ccccc67)cc5)cc4)c23)cc1 ; c1ccc(-c2c(-c3ccc(N(c4ccc(-c5ccc6c7ccc8ccccc8c7n(-c7ccccc7)c6c5)cc4)c4ccc(-c5cccc6ccccc56)cc4)cc3)ccc3ccccc23)cc1 ; c1ccc(-c2cc3ccccc3cc2-c2ccc(N(c3ccc(-c4ccc5c6ccc7ccccc7c6n(-c6ccccc6)c5c4)cc3)c3ccc(-c4cccc5ccccc45)cc3)cc2)cc1]

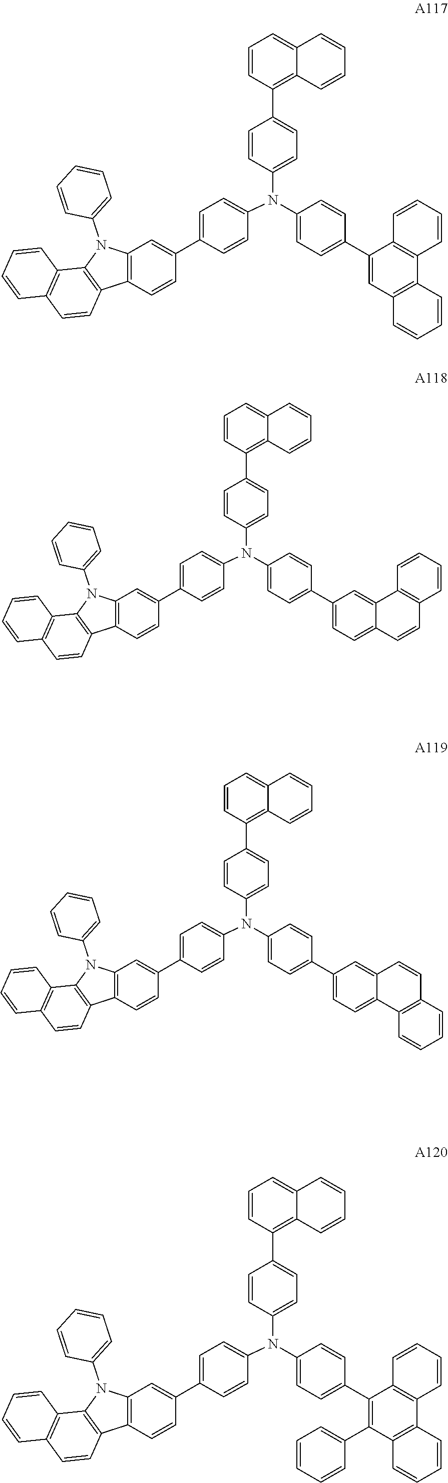 [SMILES: c1ccc(-n2c3cc(-c4ccc(N(c5ccc(-c6cccc7ccccc67)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6cccc7ccccc67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7ccccc67)cc5)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4ccc(-c5cccc6ccccc56)cc4)cc3)c3ccccc3c3ccccc23)cc1]

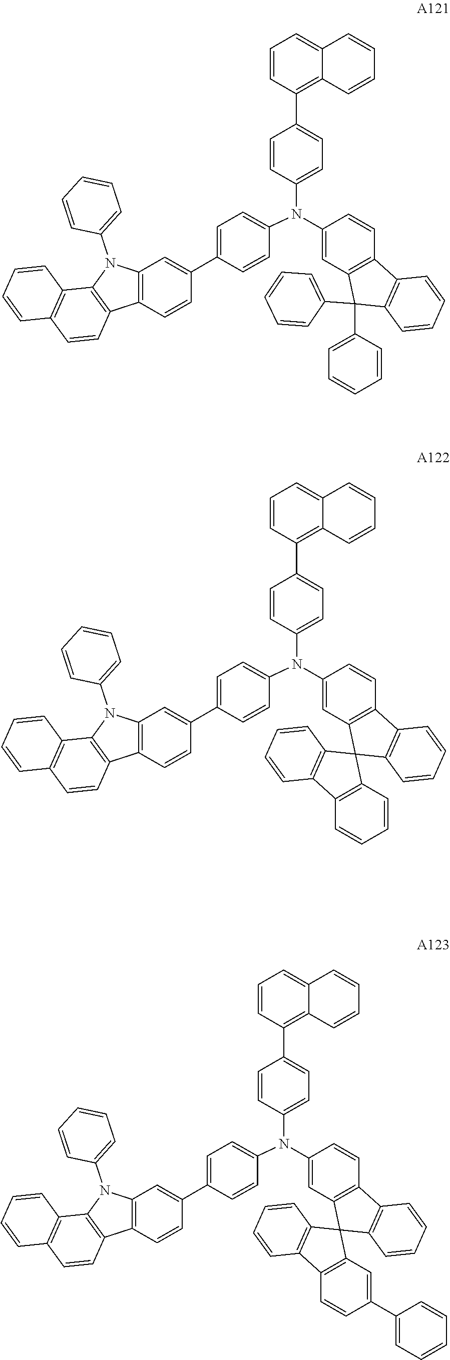 [SMILES: c1ccc(-n2c3cc(-c4ccc(N(c5ccc(-c6cccc7ccccc67)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6cccc7ccccc67)cc5)c5ccc6c(c5)C5(c7ccccc7-c7ccccc75)c5ccccc5-6)cc4)ccc3c3ccc4ccccc4c32)cc1 ; c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4ccc(-c5cccc6ccccc56)cc4)cc32)cc1]

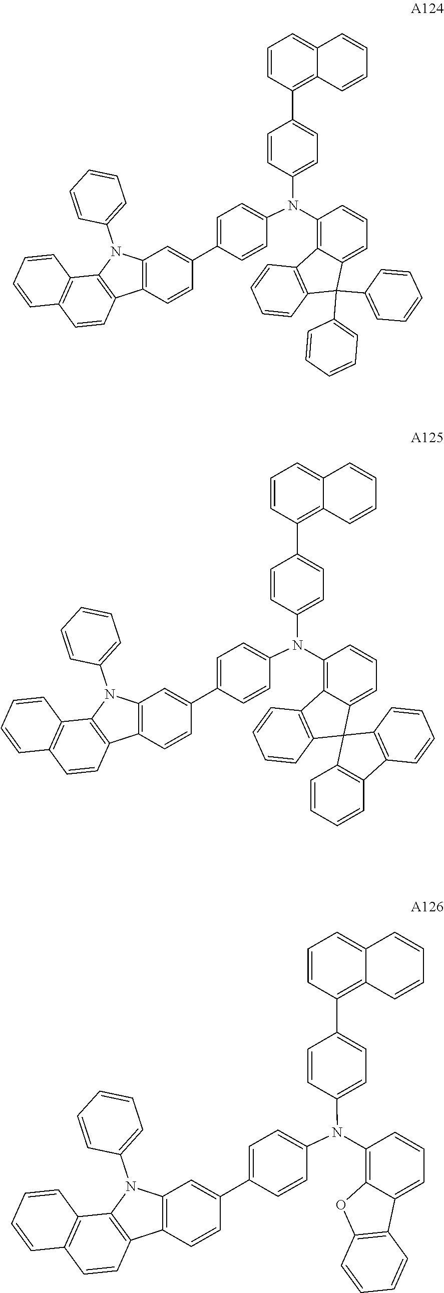 [SMILES: c1ccc(-n2c3cc(-c4ccc(N(c5ccc(-c6cccc7ccccc67)cc5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc(-c6cccc7ccccc67)cc5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc(-c6cccc7ccccc67)cc5)c5cccc6c5oc5ccccc56)cc4)ccc3c3ccc4ccccc4c32)cc1]

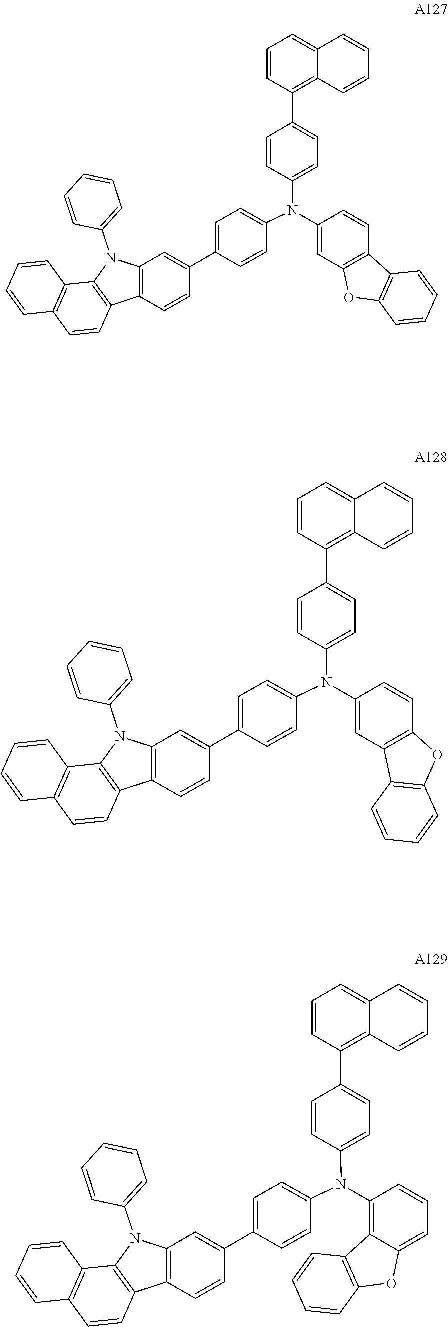 [SMILES: c1ccc(-n2c3cc(-c4ccc(N(c5ccc(-c6cccc7ccccc67)cc5)c5ccc6c(c5)oc5ccccc56)cc4)ccc3c3ccc4ccccc4c32)cc1 ; c1ccc(-n2c3cc(-c4ccc(N(c5ccc(-c6cccc7ccccc67)cc5)c5ccc6oc7ccccc7c6c5)cc4)ccc3c3ccc4ccccc4c32)cc1 ; c1ccc(-n2c3cc(-c4ccc(N(c5ccc(-c6cccc7ccccc67)cc5)c5cccc6oc7ccccc7c56)cc4)ccc3c3ccc4ccccc4c32)cc1]

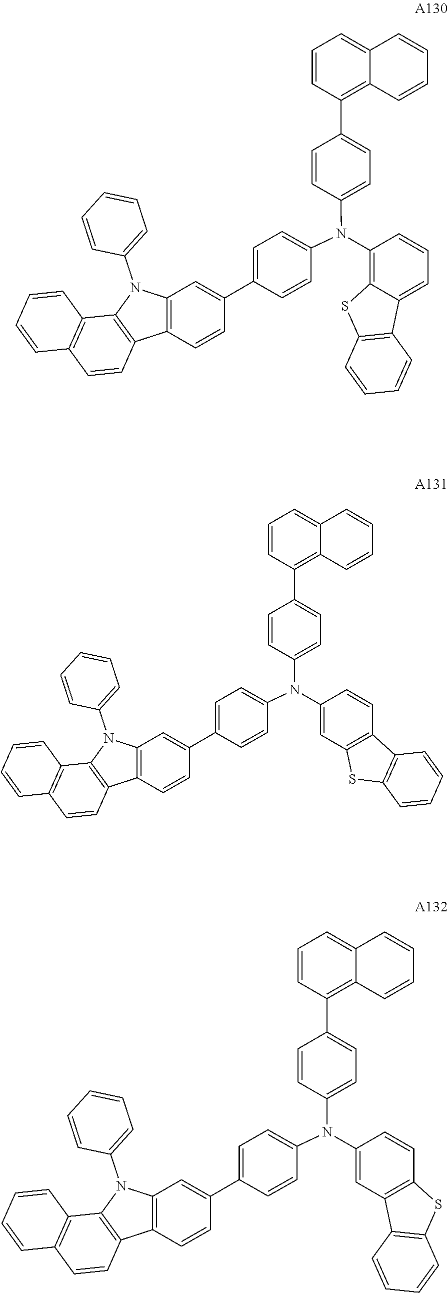 [SMILES: c1ccc(-n2c3cc(-c4ccc(N(c5ccc(-c6cccc7ccccc67)cc5)c5cccc6c5sc5ccccc56)cc4)ccc3c3ccc4ccccc4c32)cc1 ; c1ccc(-n2c3cc(-c4ccc(N(c5ccc(-c6cccc7ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6cccc7ccccc67)cc5)c5ccc6sc7ccccc7c6c5)cc4)ccc3c3ccc4ccccc4c32)cc1]

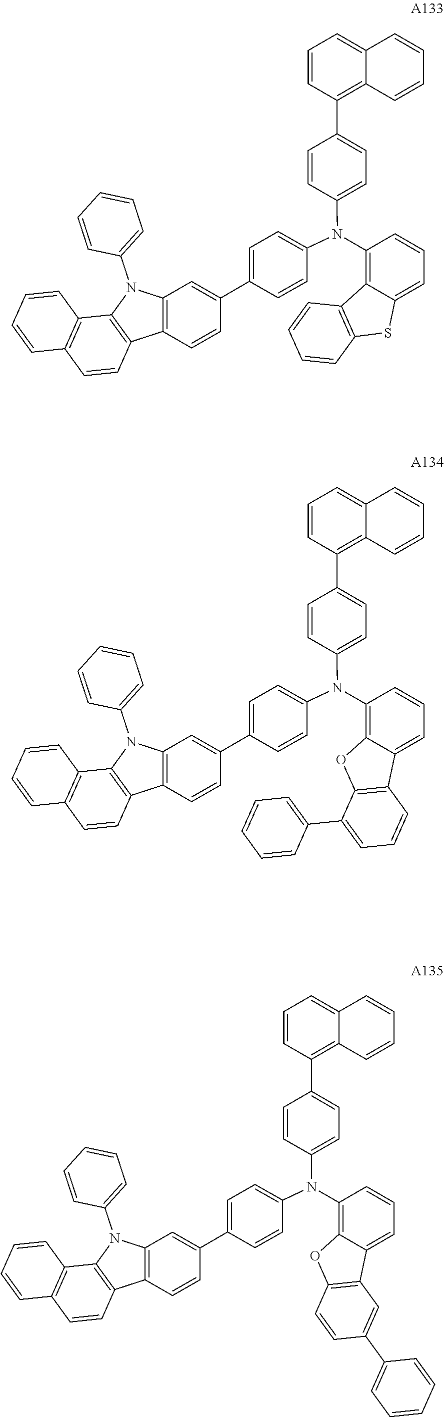 [SMILES: c1ccc(-n2c3cc(-c4ccc(N(c5ccc(-c6cccc7ccccc67)cc5)c5cccc6sc7ccccc7c56)cc4)ccc3c3ccc4ccccc4c32)cc1 ; c1ccc(-c2cccc3c2oc2c(N(c4ccc(-c5ccc6c7ccc8ccccc8c7n(-c7ccccc7)c6c5)cc4)c4ccc(-c5cccc6ccccc56)cc4)cccc23)cc1 ; c1ccc(-c2ccc3oc4c(N(c5ccc(-c6ccc7c8ccc9ccccc9c8n(-c8ccccc8)c7c6)cc5)c5ccc(-c6cccc7ccccc67)cc5)cccc4c3c2)cc1]

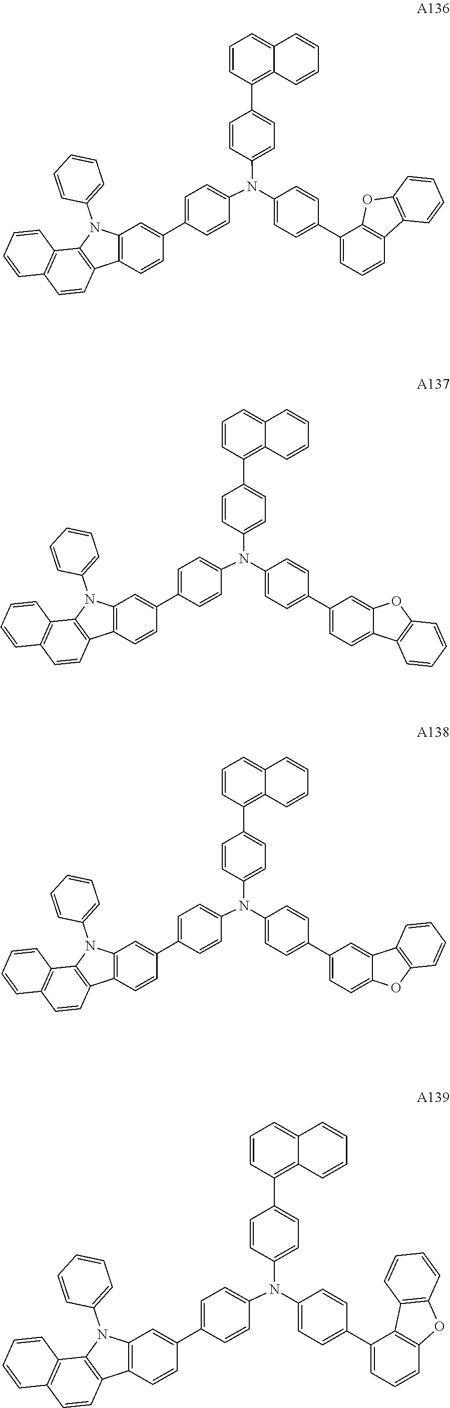 [SMILES: c1ccc(-n2c3cc(-c4ccc(N(c5ccc(-c6cccc7ccccc67)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7c(c6)oc6ccccc67)cc5)c5ccc(-c6cccc7ccccc67)cc5)cc4)ccc3c3ccc4ccccc4c32)cc1 ; c1ccc(-n2c3cc(-c4ccc(N(c5ccc(-c6ccc7oc8ccccc8c7c6)cc5)c5ccc(-c6cccc7ccccc67)cc5)cc4)ccc3c3ccc4ccccc4c32)cc1 ; c1ccc(-n2c3cc(-c4ccc(N(c5ccc(-c6cccc7ccccc67)cc5)c5ccc(-c6cccc7oc8ccccc8c67)cc5)cc4)ccc3c3ccc4ccccc4c32)cc1]

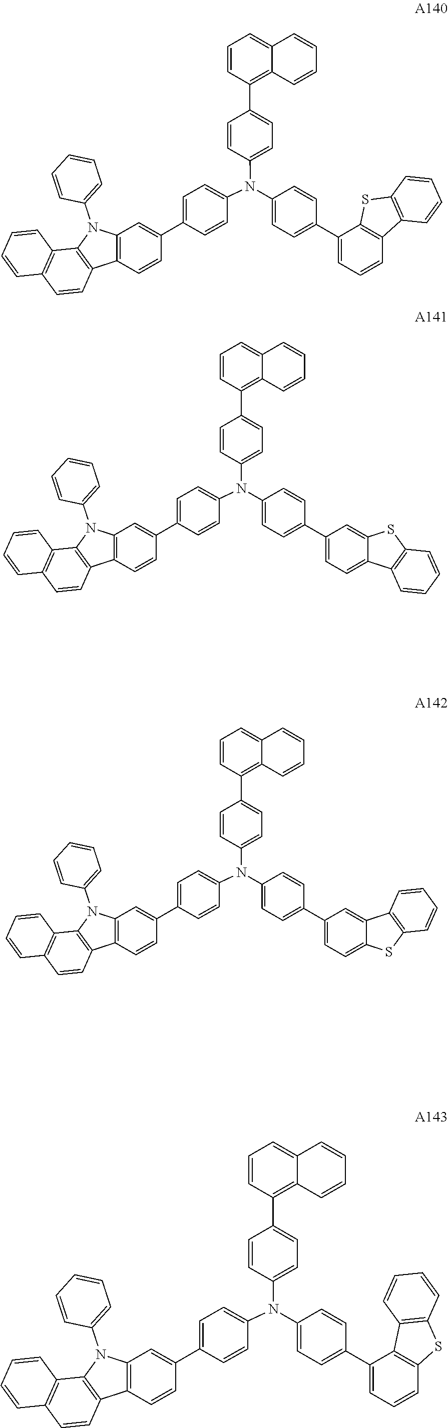 [SMILES: c1ccc(-n2c3cc(-c4ccc(N(c5ccc(-c6cccc7ccccc67)cc5)c5ccc(-c6cccc7c6sc6ccccc67)cc5)cc4)ccc3c3ccc4ccccc4c32)cc1 ; c1ccc(-n2c3cc(-c4ccc(N(c5ccc(-c6ccc7c(c6)sc6ccccc67)cc5)c5ccc(-c6cccc7ccccc67)cc5)cc4)ccc3c3ccc4ccccc4c32)cc1 ; c1ccc(-n2c3cc(-c4ccc(N(c5ccc(-c6ccc7sc8ccccc8c7c6)cc5)c5ccc(-c6cccc7ccccc67)cc5)cc4)ccc3c3ccc4ccccc4c32)cc1 ; c1ccc(-n2c3cc(-c4ccc(N(c5ccc(-c6cccc7ccccc67)cc5)c5ccc(-c6cccc7sc8ccccc8c67)cc5)cc4)ccc3c3ccc4ccccc4c32)cc1]

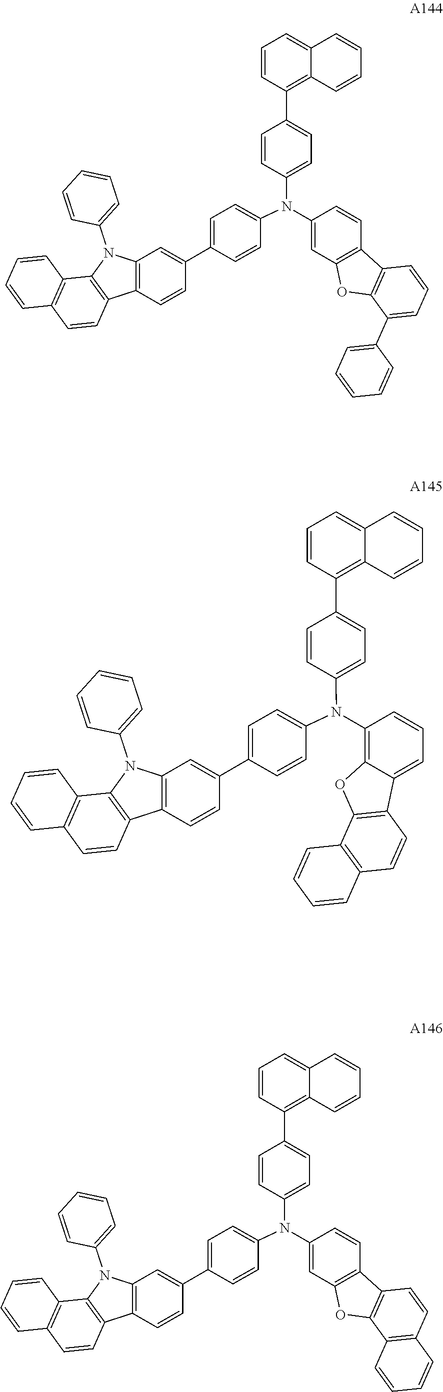 [SMILES: c1ccc(-c2cccc3c2oc2cc(N(c4ccc(-c5ccc6c7ccc8ccccc8c7n(-c7ccccc7)c6c5)cc4)c4ccc(-c5cccc6ccccc56)cc4)ccc23)cc1 ; c1ccc(-n2c3cc(-c4ccc(N(c5ccc(-c6cccc7ccccc67)cc5)c5cccc6c5oc5c7ccccc7ccc65)cc4)ccc3c3ccc4ccccc4c32)cc1 ; c1ccc(-n2c3cc(-c4ccc(N(c5ccc(-c6cccc7ccccc67)cc5)c5ccc6c(c5)oc5c7ccccc7ccc65)cc4)ccc3c3ccc4ccccc4c32)cc1]

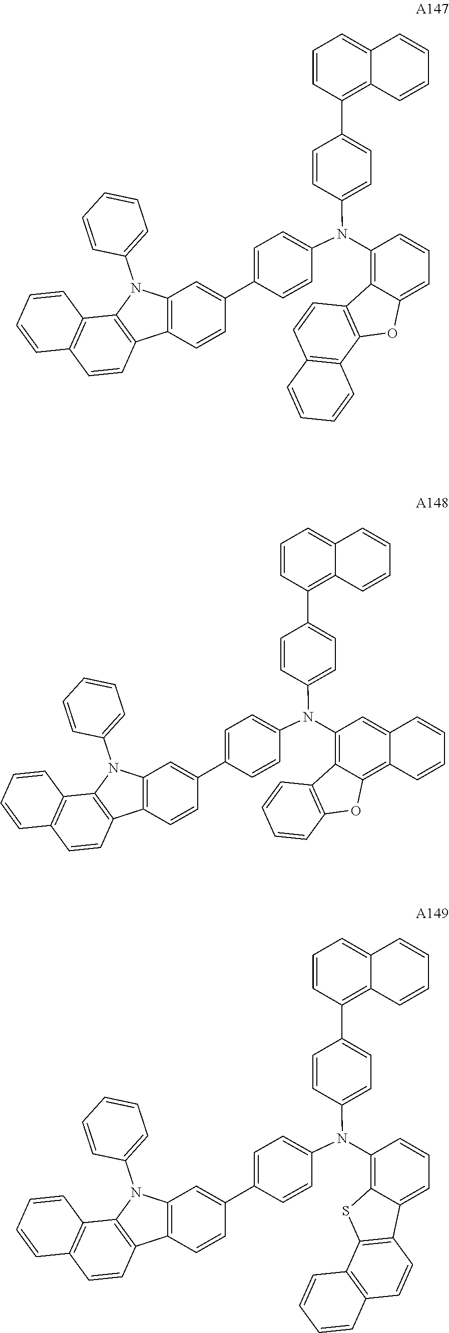 [SMILES: c1ccc(-n2c3cc(-c4ccc(N(c5ccc(-c6cccc7ccccc67)cc5)c5cccc6oc7c8ccccc8ccc7c56)cc4)ccc3c3ccc4ccccc4c32)cc1 ; c1ccc(-n2c3cc(-c4ccc(N(c5ccc(-c6cccc7ccccc67)cc5)c5cc6ccccc6c6oc7ccccc7c56)cc4)ccc3c3ccc4ccccc4c32)cc1 ; c1ccc(-n2c3cc(-c4ccc(N(c5ccc(-c6cccc7ccccc67)cc5)c5cccc6c5sc5c7ccccc7ccc65)cc4)ccc3c3ccc4ccccc4c32)cc1]

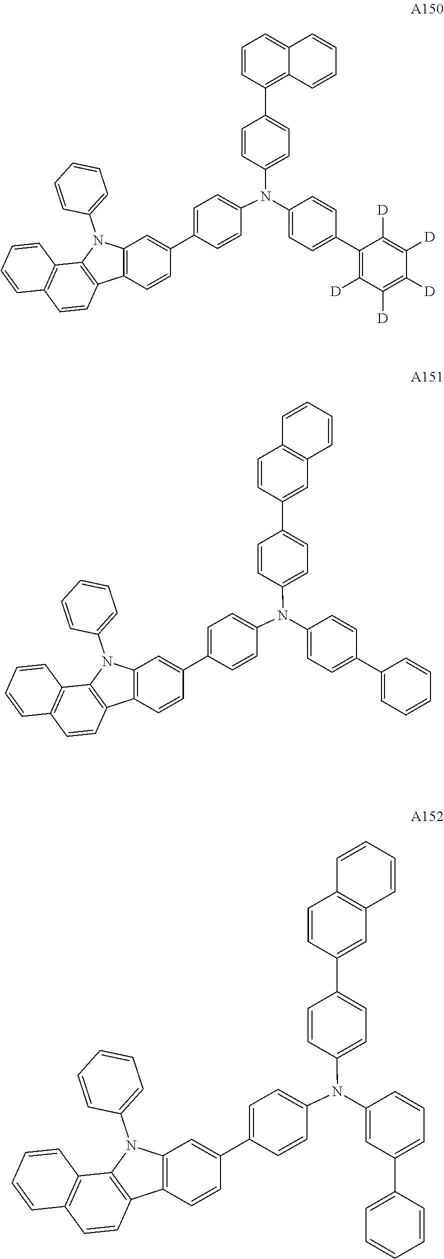 [SMILES: [2H]c1c([2H])c([2H])c(-c2ccc(N(c3ccc(-c4ccc5c6ccc7ccccc7c6n(-c6ccccc6)c5c4)cc3)c3ccc(-c4cccc5ccccc45)cc3)cc2)c([2H])c1[2H] ; c1ccc(-c2ccc(N(c3ccc(-c4ccc5ccccc5c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5ccccc5c4)cc3)c3ccc(-c4ccc5c6ccc7ccccc7c6n(-c6ccccc6)c5c4)cc3)c2)cc1]

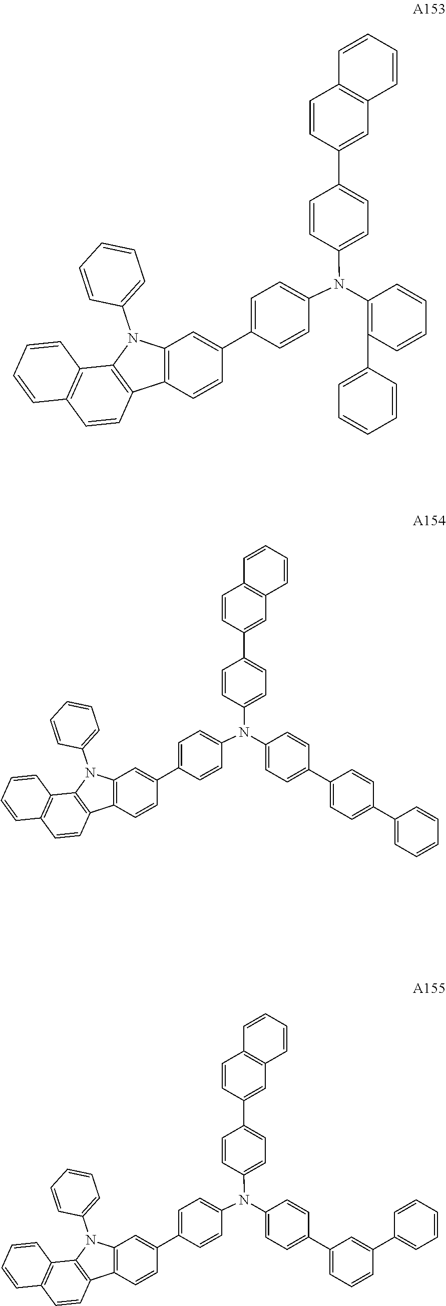 [SMILES: c1ccc(-c2ccccc2N(c2ccc(-c3ccc4ccccc4c3)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6ccccc6c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6ccccc6c5)cc4)c4ccc(-c5ccc6c7ccc8ccccc8c7n(-c7ccccc7)c6c5)cc4)cc3)c2)cc1]

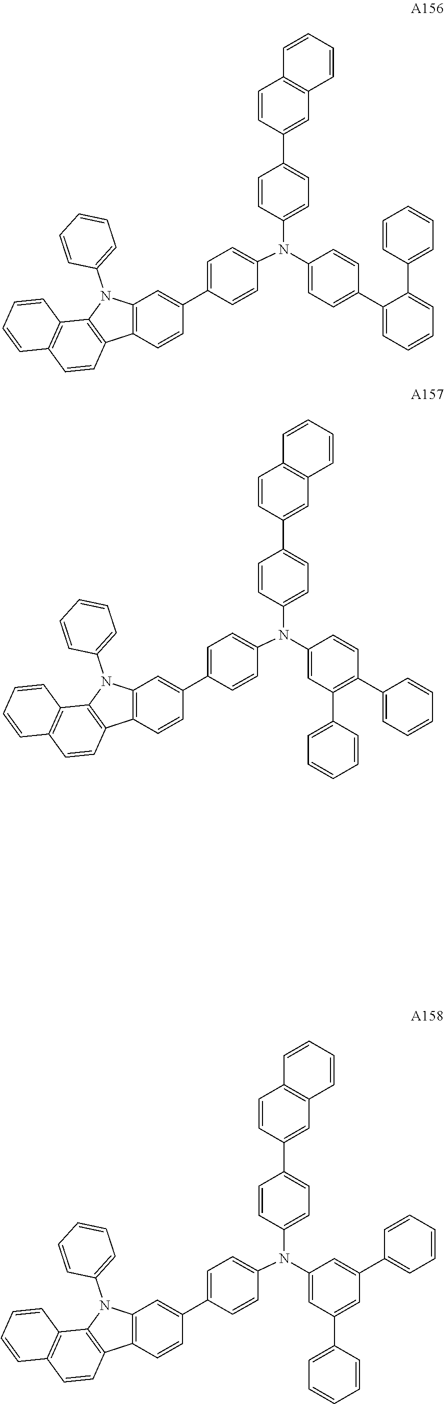 [SMILES: c1ccc(-c2ccccc2-c2ccc(N(c3ccc(-c4ccc5ccccc5c4)cc3)c3ccc(-c4ccc5c6ccc7ccccc7c6n(-c6ccccc6)c5c4)cc3)cc2)cc1 ; c1ccc(-c2ccc(N(c3ccc(-c4ccc5ccccc5c4)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5ccccc5c4)cc3)c3ccc(-c4ccc5c6ccc7ccccc7c6n(-c6ccccc6)c5c4)cc3)c2)cc1]

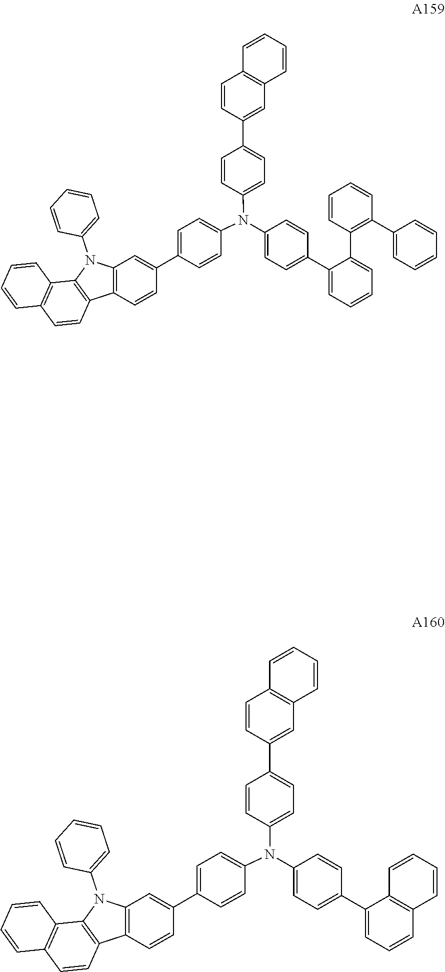 [SMILES: c1ccc(-c2ccccc2-c2ccccc2-c2ccc(N(c3ccc(-c4ccc5ccccc5c4)cc3)c3ccc(-c4ccc5c6ccc7ccccc7c6n(-c6ccccc6)c5c4)cc3)cc2)cc1 ; c1ccc(-n2c3cc(-c4ccc(N(c5ccc(-c6ccc7ccccc7c6)cc5)c5ccc(-c6cccc7ccccc67)cc5)cc4)ccc3c3ccc4ccccc4c32)cc1]

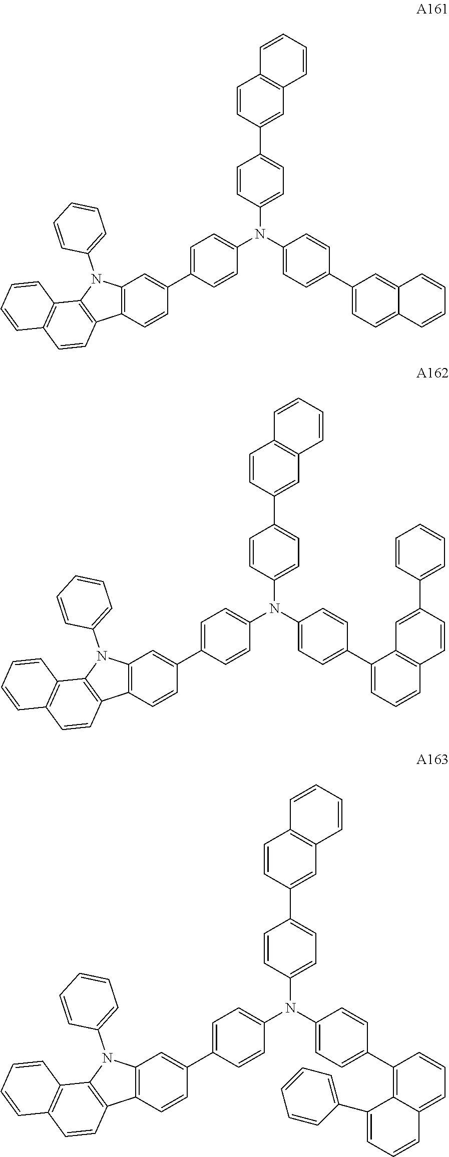 [SMILES: c1ccc(-n2c3cc(-c4ccc(N(c5ccc(-c6ccc7ccccc7c6)cc5)c5ccc(-c6ccc7ccccc7c6)cc5)cc4)ccc3c3ccc4ccccc4c32)cc1 ; c1ccc(-c2ccc3cccc(-c4ccc(N(c5ccc(-c6ccc7ccccc7c6)cc5)c5ccc(-c6ccc7c8ccc9ccccc9c8n(-c8ccccc8)c7c6)cc5)cc4)c3c2)cc1 ; c1ccc(-c2cccc3cccc(-c4ccc(N(c5ccc(-c6ccc7ccccc7c6)cc5)c5ccc(-c6ccc7c8ccc9ccccc9c8n(-c8ccccc8)c7c6)cc5)cc4)c23)cc1]

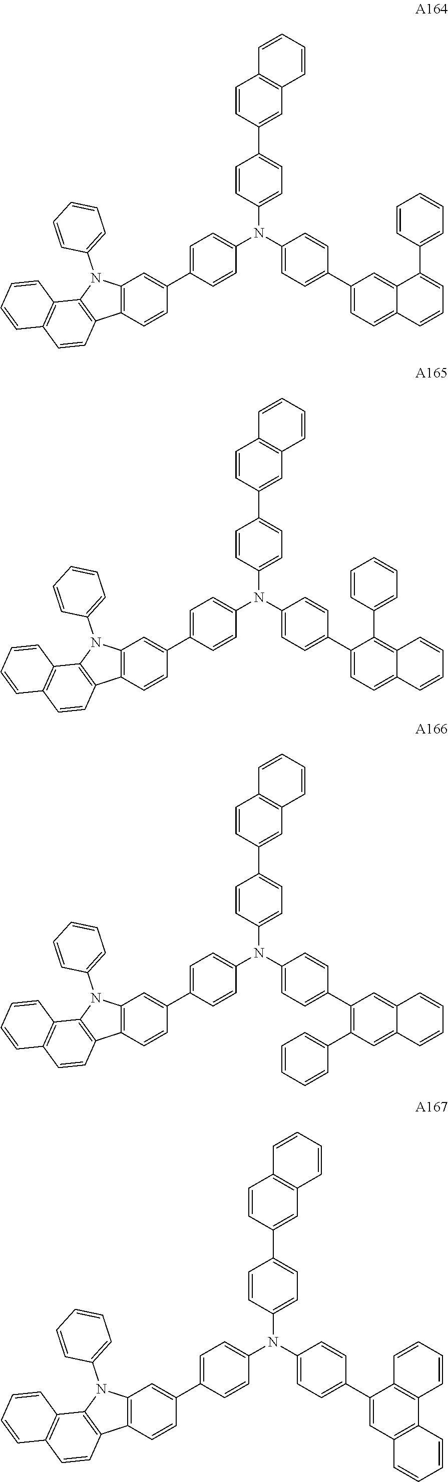 [SMILES: c1ccc(-c2cccc3ccc(-c4ccc(N(c5ccc(-c6ccc7ccccc7c6)cc5)c5ccc(-c6ccc7c8ccc9ccccc9c8n(-c8ccccc8)c7c6)cc5)cc4)cc23)cc1 ; c1ccc(-c2c(-c3ccc(N(c4ccc(-c5ccc6ccccc6c5)cc4)c4ccc(-c5ccc6c7ccc8ccccc8c7n(-c7ccccc7)c6c5)cc4)cc3)ccc3ccccc23)cc1 ; c1ccc(-c2cc3ccccc3cc2-c2ccc(N(c3ccc(-c4ccc5ccccc5c4)cc3)c3ccc(-c4ccc5c6ccc7ccccc7c6n(-c6ccccc6)c5c4)cc3)cc2)cc1 ; c1ccc(-n2c3cc(-c4ccc(N(c5ccc(-c6ccc7ccccc7c6)cc5)c5ccc(-c6cc7ccccc7c7ccccc67)cc5)cc4)ccc3c3ccc4ccccc4c32)cc1]

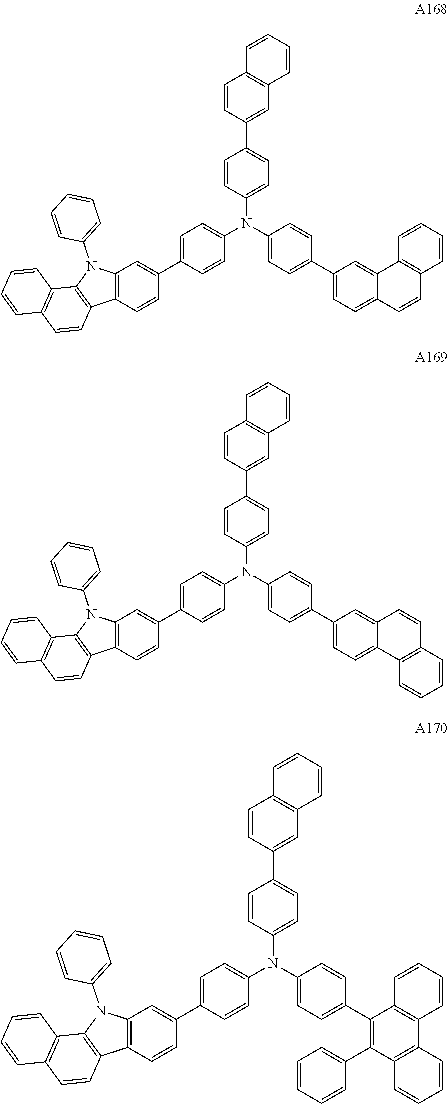 [SMILES: c1ccc(-n2c3cc(-c4ccc(N(c5ccc(-c6ccc7ccccc7c6)cc5)c5ccc(-c6ccc7ccc8ccccc8c7c6)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6ccc7c(ccc8ccccc87)c6)cc5)cc4)ccc3c3ccc4ccccc4c32)cc1 ; c1ccc(-c2c(-c3ccc(N(c4ccc(-c5ccc6ccccc6c5)cc4)c4ccc(-c5ccc6c7ccc8ccccc8c7n(-c7ccccc7)c6c5)cc4)cc3)c3ccccc3c3ccccc23)cc1]

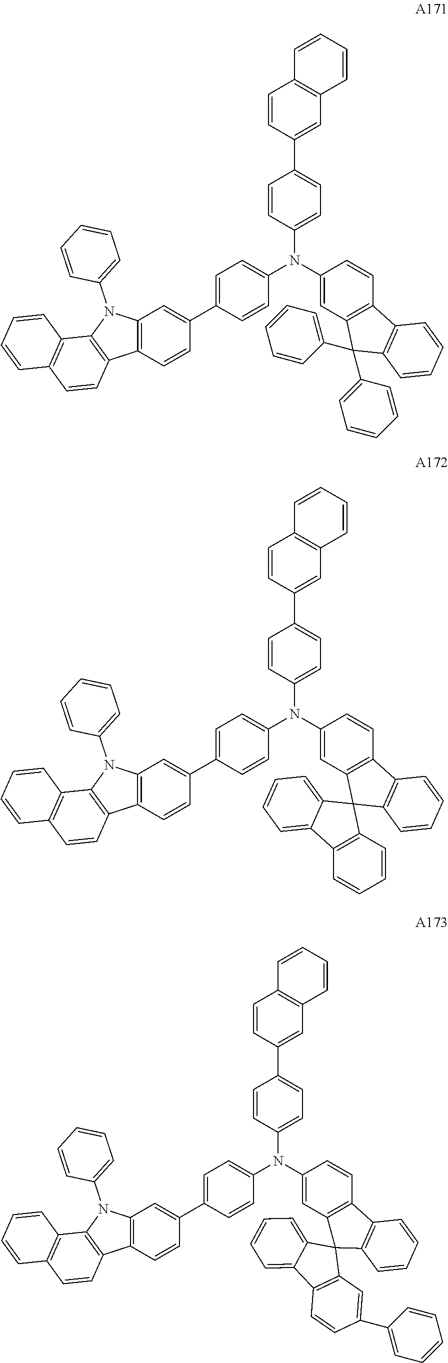 [SMILES: c1ccc(-n2c3cc(-c4ccc(N(c5ccc(-c6ccc7ccccc7c6)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6ccc7ccccc7c6)cc5)c5ccc6c(c5)C5(c7ccccc7-c7ccccc75)c5ccccc5-6)cc4)ccc3c3ccc4ccccc4c32)cc1 ; c1ccc(-c2ccc3c(c2)C2(c4ccccc4-3)c3ccccc3-c3ccc(N(c4ccc(-c5ccc6ccccc6c5)cc4)c4ccc(-c5ccc6c7ccc8ccccc8c7n(-c7ccccc7)c6c5)cc4)cc32)cc1]

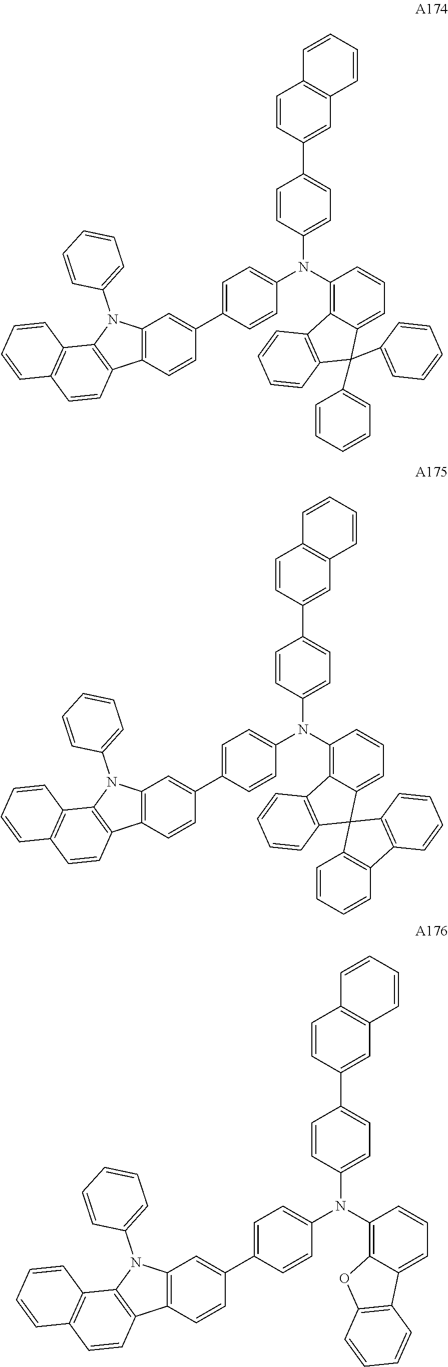 [SMILES: c1ccc(-n2c3cc(-c4ccc(N(c5ccc(-c6ccc7ccccc7c6)cc5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc(-c6ccc7ccccc7c6)cc5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc(-c6ccc7ccccc7c6)cc5)c5cccc6c5oc5ccccc56)cc4)ccc3c3ccc4ccccc4c32)cc1]

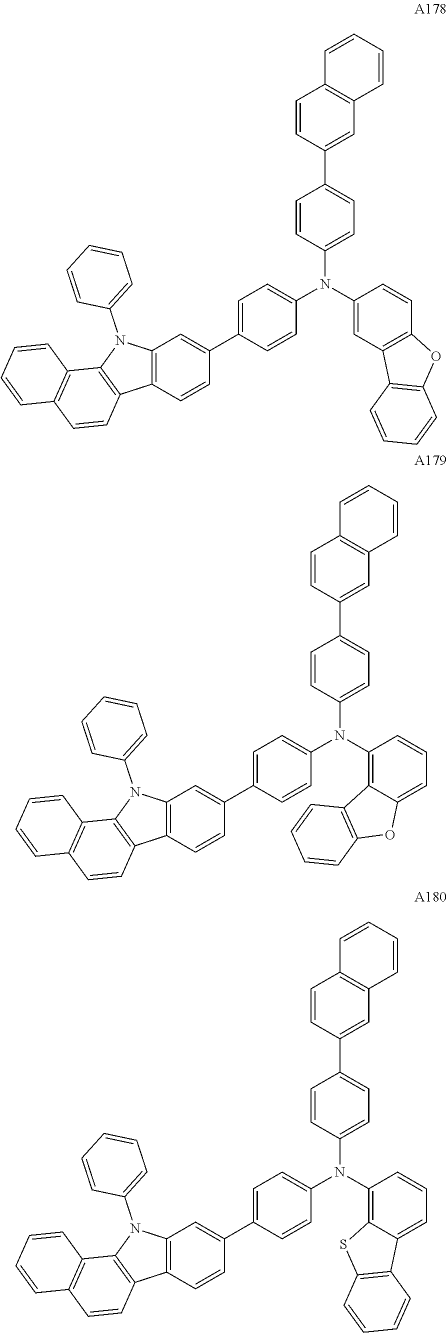 [SMILES: c1ccc(-n2c3cc(-c4ccc(N(c5ccc(-c6ccc7ccccc7c6)cc5)c5ccc6oc7ccccc7c6c5)cc4)ccc3c3ccc4ccccc4c32)cc1 ; c1ccc(-n2c3cc(-c4ccc(N(c5ccc(-c6ccc7ccccc7c6)cc5)c5cccc6oc7ccccc7c56)cc4)ccc3c3ccc4ccccc4c32)cc1 ; c1ccc(-n2c3cc(-c4ccc(N(c5ccc(-c6ccc7ccccc7c6)cc5)c5cccc6c5sc5ccccc56)cc4)ccc3c3ccc4ccccc4c32)cc1]

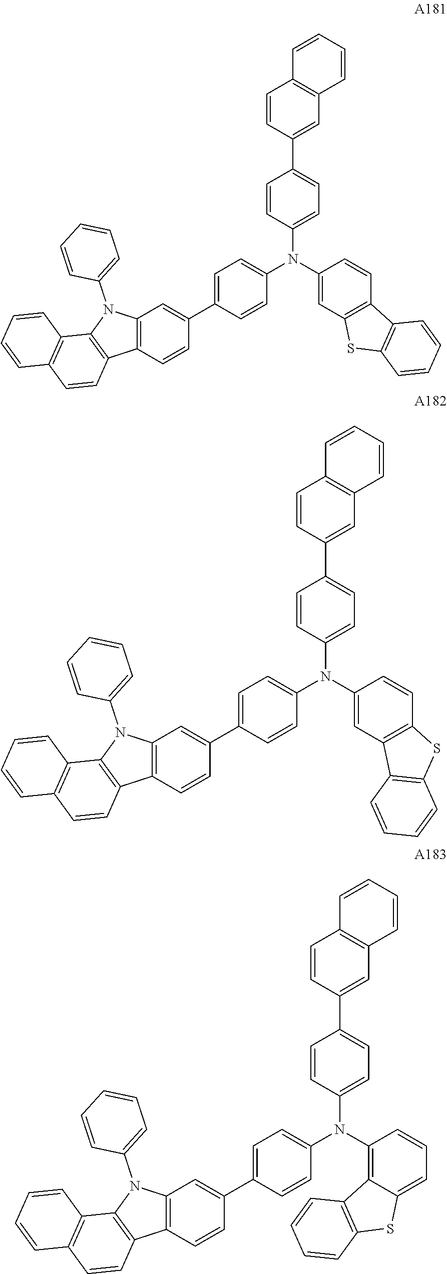 [SMILES: c1ccc(-n2c3cc(-c4ccc(N(c5ccc(-c6ccc7ccccc7c6)cc5)c5ccc6c(c5)sc5ccccc56)cc4)ccc3c3ccc4ccccc4c32)cc1 ; c1ccc(-n2c3cc(-c4ccc(N(c5ccc(-c6ccc7ccccc7c6)cc5)c5ccc6sc7ccccc7c6c5)cc4)ccc3c3ccc4ccccc4c32)cc1 ; c1ccc(-n2c3cc(-c4ccc(N(c5ccc(-c6ccc7ccccc7c6)cc5)c5cccc6sc7ccccc7c56)cc4)ccc3c3ccc4ccccc4c32)cc1]

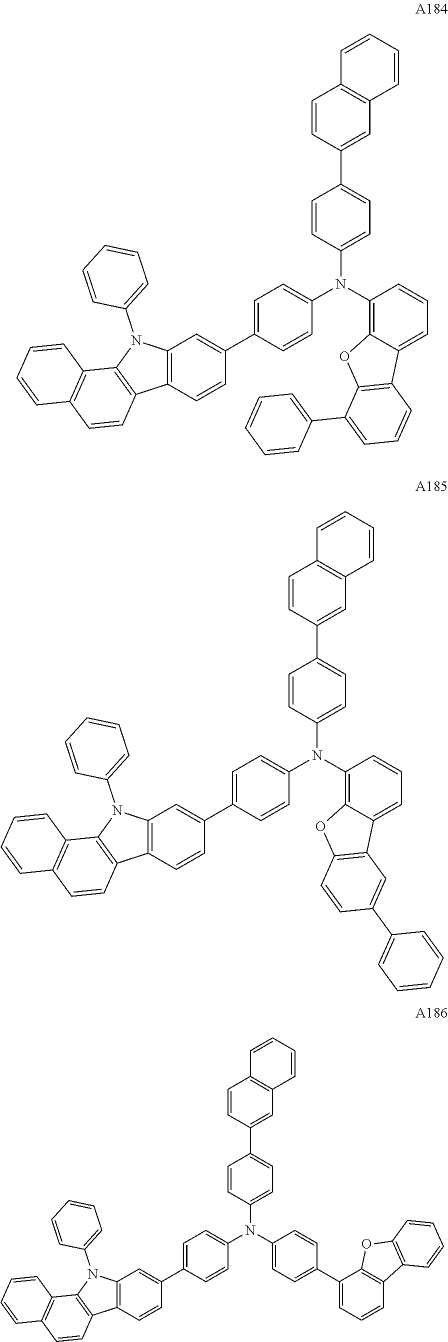 [SMILES: c1ccc(-c2cccc3c2oc2c(N(c4ccc(-c5ccc6ccccc6c5)cc4)c4ccc(-c5ccc6c7ccc8ccccc8c7n(-c7ccccc7)c6c5)cc4)cccc23)cc1 ; c1ccc(-c2ccc3oc4c(N(c5ccc(-c6ccc7ccccc7c6)cc5)c5ccc(-c6ccc7c8ccc9ccccc9c8n(-c8ccccc8)c7c6)cc5)cccc4c3c2)cc1 ; c1ccc(-n2c3cc(-c4ccc(N(c5ccc(-c6ccc7ccccc7c6)cc5)c5ccc(-c6cccc7c6oc6ccccc67)cc5)cc4)ccc3c3ccc4ccccc4c32)cc1]

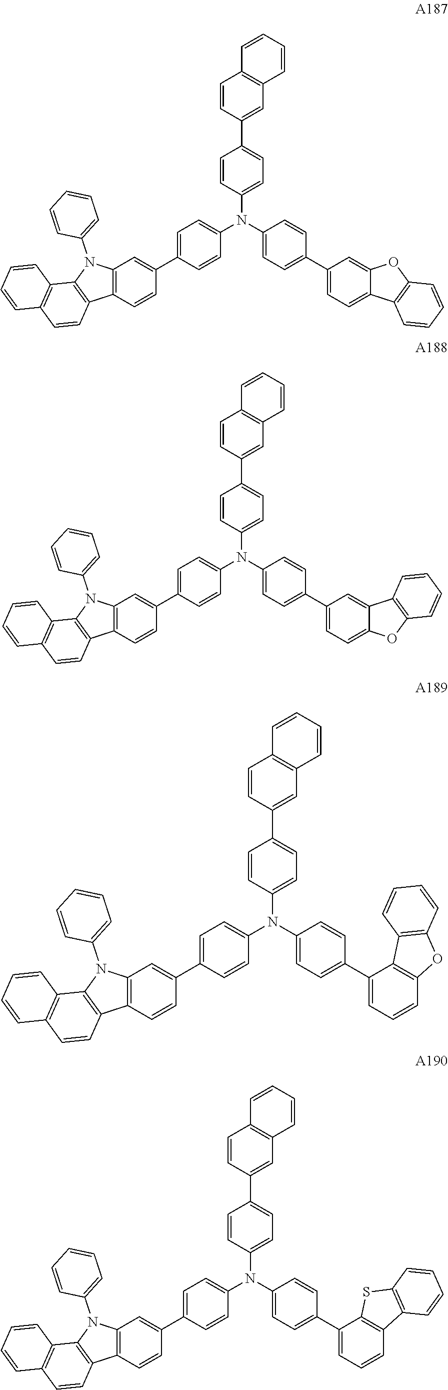 [SMILES: c1ccc(-n2c3cc(-c4ccc(N(c5ccc(-c6ccc7ccccc7c6)cc5)c5ccc(-c6ccc7c(c6)oc6ccccc67)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6ccc7oc8ccccc8c7c6)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6cccc7oc8ccccc8c67)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6cccc7c6sc6ccccc67)cc5)cc4)ccc3c3ccc4ccccc4c32)cc1]

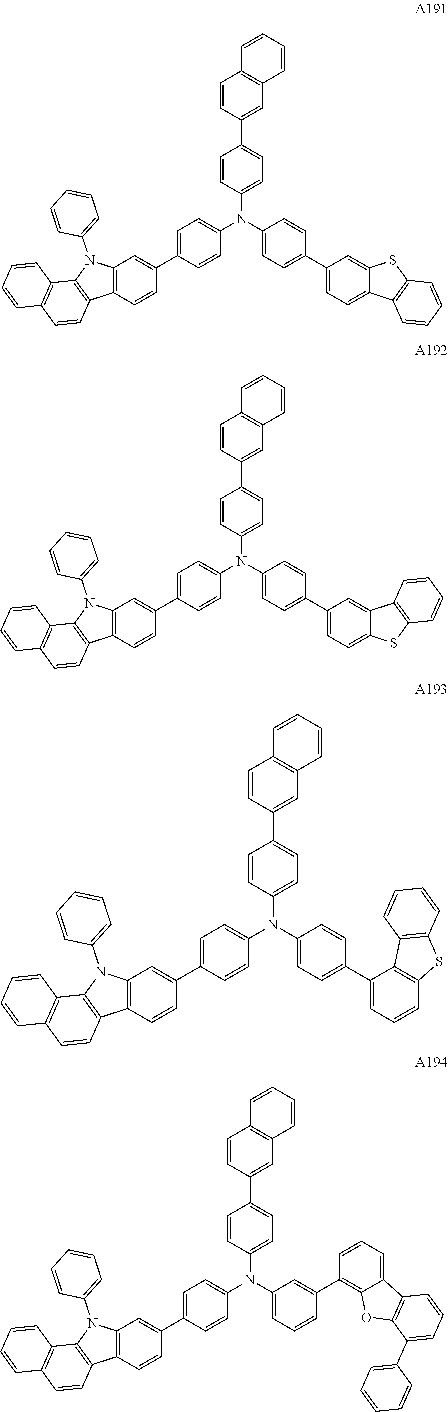 [SMILES: c1ccc(-n2c3cc(-c4ccc(N(c5ccc(-c6ccc7ccccc7c6)cc5)c5ccc(-c6ccc7c(c6)sc6ccccc67)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6ccc7sc8ccccc8c7c6)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6cccc7sc8ccccc8c67)cc5)cc4)ccc3c3ccc4ccccc4c32)cc1 ; c1ccc(-c2cccc3c2oc2c(-c4cccc(N(c5ccc(-c6ccc7ccccc7c6)cc5)c5ccc(-c6ccc7c8ccc9ccccc9c8n(-c8ccccc8)c7c6)cc5)c4)cccc23)cc1]

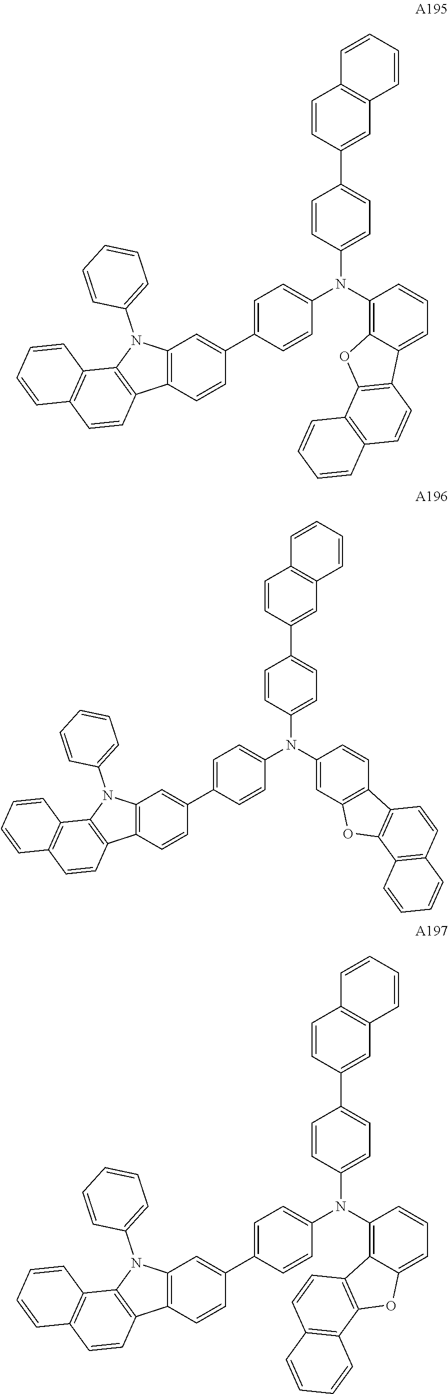

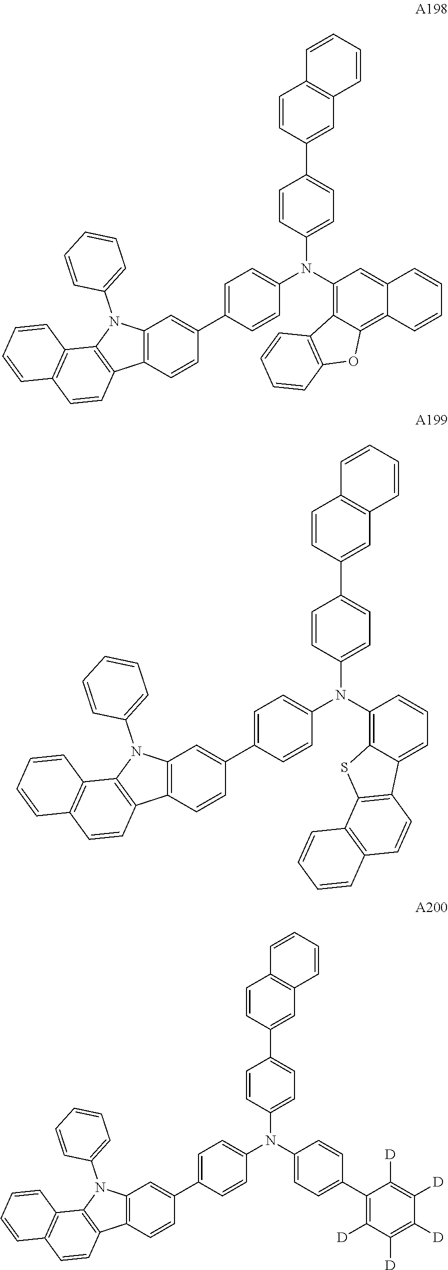 [SMILES: c1ccc(-n2c3cc(-c4ccc(N(c5ccc(-c6ccc7ccccc7c6)cc5)c5cc6ccccc6c6oc7ccccc7c56)cc4)ccc3c3ccc4ccccc4c32)cc1 ; c1ccc(-n2c3cc(-c4ccc(N(c5ccc(-c6ccc7ccccc7c6)cc5)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5ccccc5c4)cc3)c3ccc(-c4ccc5c6ccc7ccccc7c6n(-c6ccccc6)c5c4)cc3)cc2)c([2H])c1[2H]]

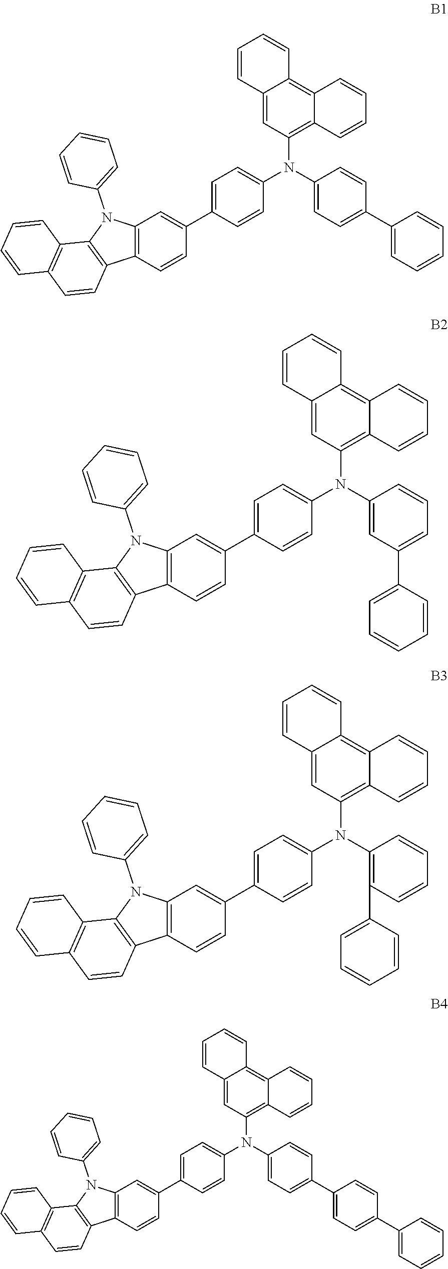 [SMILES: c1ccc(-c2ccc(N(c3ccc(-c4ccc5c6ccc7ccccc7c6n(-c6ccccc6)c5c4)cc3)c3cc4ccccc4c4ccccc34)cc2)cc1 ; c1ccc(-c2cccc(N(c3ccc(-c4ccc5c6ccc7ccccc7c6n(-c6ccccc6)c5c4)cc3)c3cc4ccccc4c4ccccc34)c2)cc1 ; c1ccc(-c2ccccc2N(c2ccc(-c3ccc4c5ccc6ccccc6c5n(-c5ccccc5)c4c3)cc2)c2cc3ccccc3c3ccccc23)cc1 ; c1ccc(-c2ccc(-c3ccc(N(c4ccc(-c5ccc6c7ccc8ccccc8c7n(-c7ccccc7)c6c5)cc4)c4cc5ccccc5c5ccccc45)cc3)cc2)cc1]

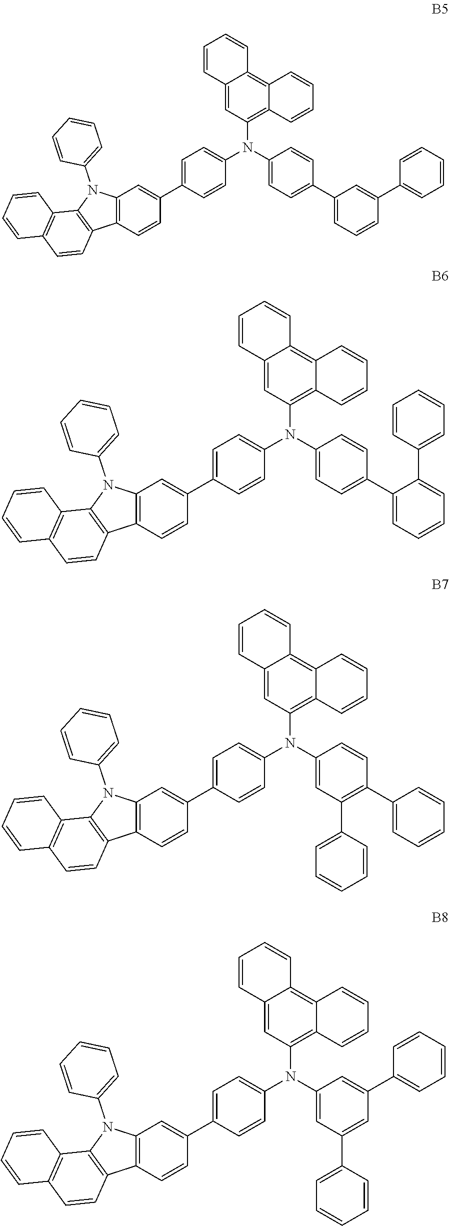 [SMILES: c1ccc(-c2cccc(-c3ccc(N(c4ccc(-c5ccc6c7ccc8ccccc8c7n(-c7ccccc7)c6c5)cc4)c4cc5ccccc5c5ccccc45)cc3)c2)cc1 ; c1ccc(-c2ccccc2-c2ccc(N(c3ccc(-c4ccc5c6ccc7ccccc7c6n(-c6ccccc6)c5c4)cc3)c3cc4ccccc4c4ccccc34)cc2)cc1 ; c1ccc(-c2ccc(N(c3ccc(-c4ccc5c6ccc7ccccc7c6n(-c6ccccc6)c5c4)cc3)c3cc4ccccc4c4ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cc4ccccc4c4ccccc34)c2)cc1]

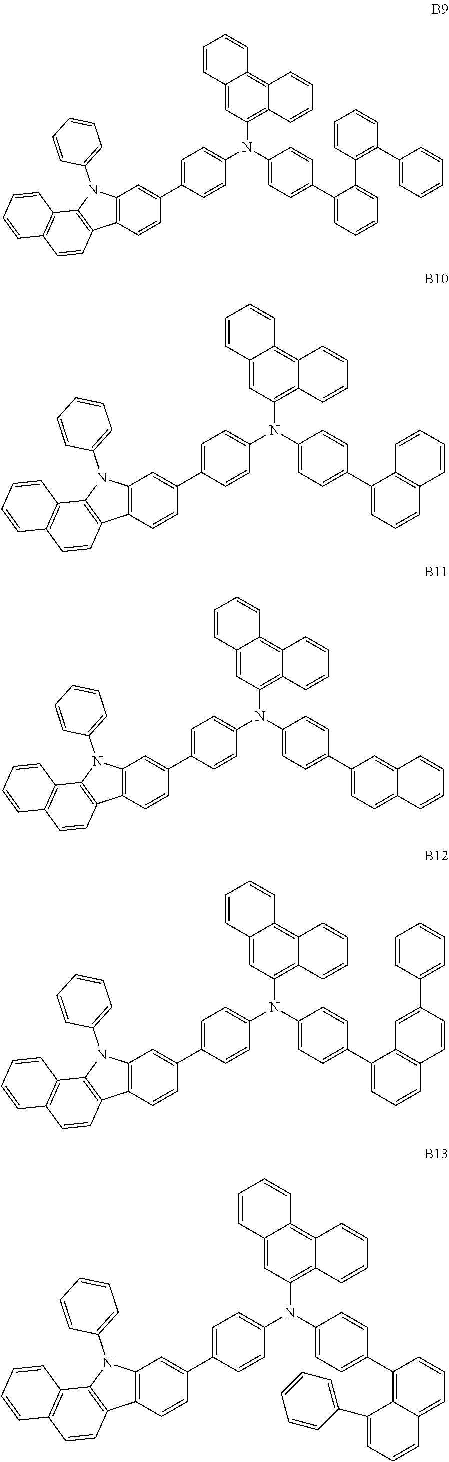

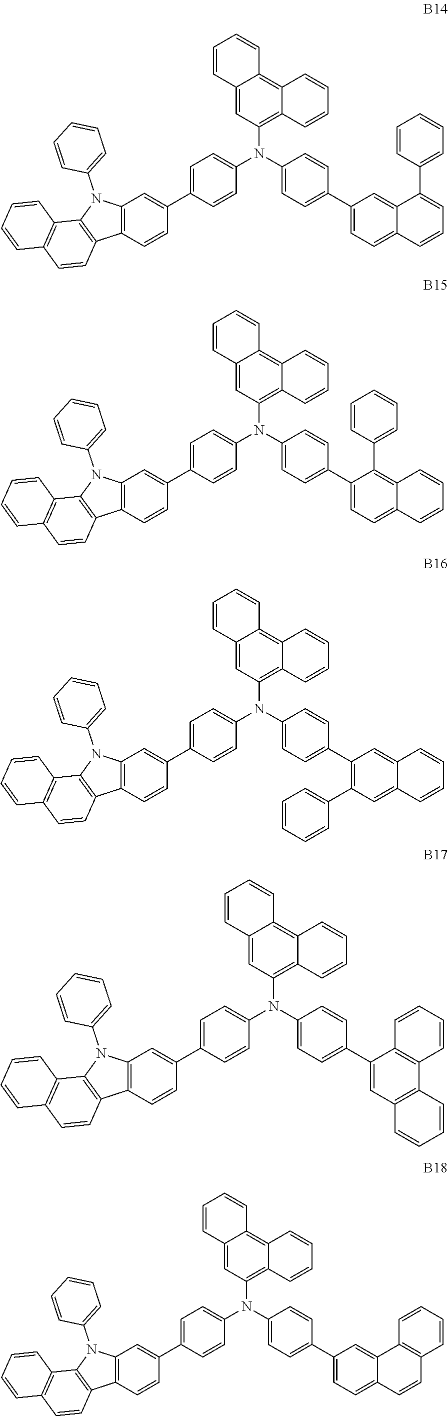

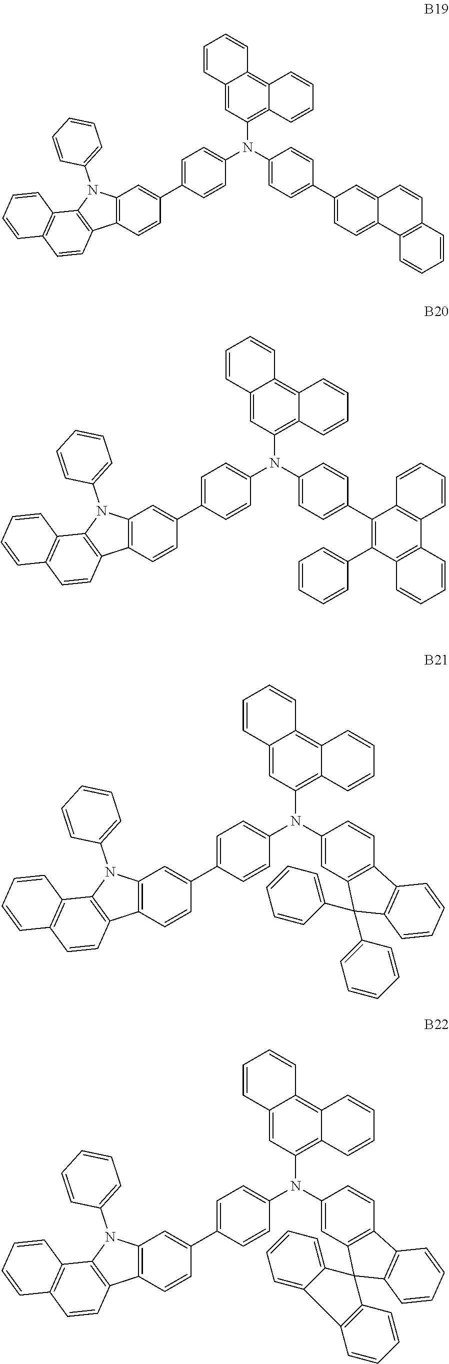 [SMILES: c1ccc(-n2c3cc(-c4ccc(N(c5ccc(-c6ccc7c(ccc8ccccc87)c6)cc5)c5cc6ccccc6c6ccccc56)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4cc5ccccc5c5ccccc45)cc3)c3ccccc3c3ccccc23)cc1 ; c1ccc(-n2c3cc(-c4ccc(N(c5ccc6c(c5)C(c5ccccc5)(c5ccccc5)c5ccccc5-6)c5cc6ccccc6c6ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5cc6ccccc6c6ccccc56)cc4)ccc3c3ccc4ccccc4c32)cc1]

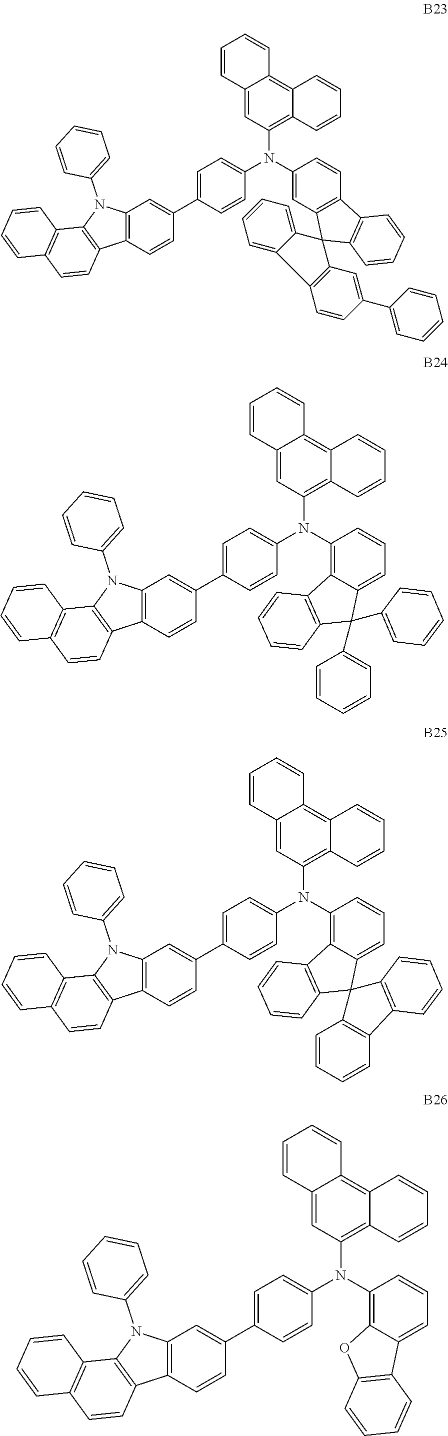 [SMILES: c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4cc5ccccc5c5ccccc45)cc32)cc1 ; c1ccc(-n2c3cc(-c4ccc(N(c5cccc6c5-c5ccccc5C6(c5ccccc5)c5ccccc5)c5cc6ccccc6c6ccccc56)cc4)ccc3c3ccc4ccccc4c32)cc1 ; c1ccc(-n2c3cc(-c4ccc(N(c5cccc6c5-c5ccccc5C65c6ccccc6-c6ccccc65)c5cc6ccccc6c6ccccc56)cc4)ccc3c3ccc4ccccc4c32)cc1 ; c1ccc(-n2c3cc(-c4ccc(N(c5cc6ccccc6c6ccccc56)c5cccc6c5oc5ccccc56)cc4)ccc3c3ccc4ccccc4c32)cc1]

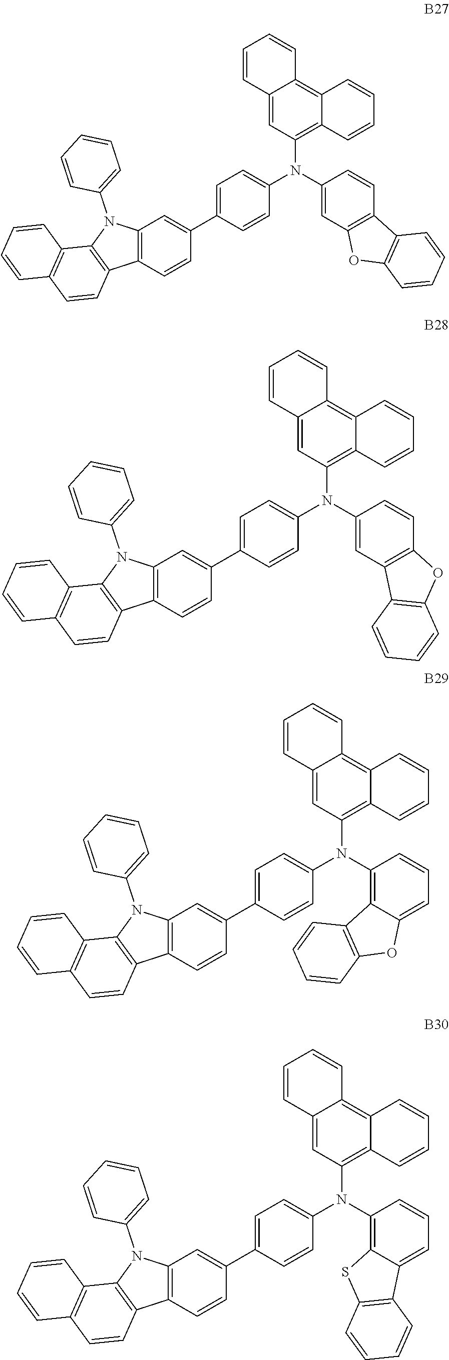 [SMILES: c1ccc(-n2c3cc(-c4ccc(N(c5ccc6c(c5)oc5ccccc56)c5cc6ccccc6c6ccccc56)cc4)ccc3c3ccc4ccccc4c32)cc1 ; c1ccc(-n2c3cc(-c4ccc(N(c5ccc6oc7ccccc7c6c5)c5cc6ccccc6c6ccccc56)cc4)ccc3c3ccc4ccccc4c32)cc1 ; c1ccc(-n2c3cc(-c4ccc(N(c5cc6ccccc6c6ccccc56)c5cccc6oc7ccccc7c56)cc4)ccc3c3ccc4ccccc4c32)cc1 ; c1ccc(-n2c3cc(-c4ccc(N(c5cc6ccccc6c6ccccc56)c5cccc6c5sc5ccccc56)cc4)ccc3c3ccc4ccccc4c32)cc1]

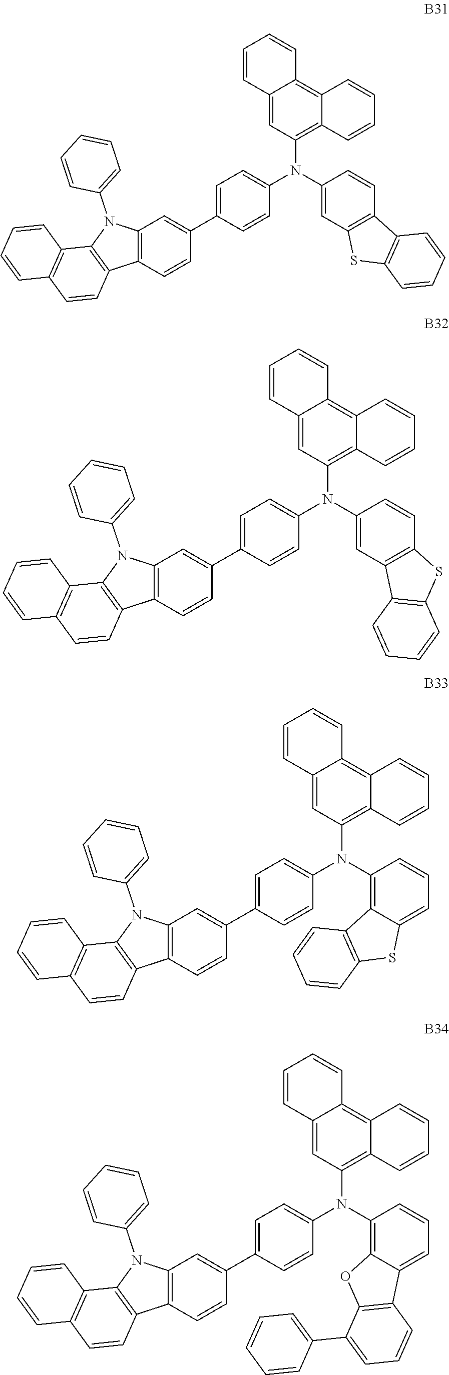 [SMILES: c1ccc(-n2c3cc(-c4ccc(N(c5ccc6c(c5)sc5ccccc56)c5cc6ccccc6c6ccccc56)cc4)ccc3c3ccc4ccccc4c32)cc1 ; c1ccc(-n2c3cc(-c4ccc(N(c5ccc6sc7ccccc7c6c5)c5cc6ccccc6c6ccccc56)cc4)ccc3c3ccc4ccccc4c32)cc1 ; c1ccc(-n2c3cc(-c4ccc(N(c5cc6ccccc6c6ccccc56)c5cccc6sc7ccccc7c56)cc4)ccc3c3ccc4ccccc4c32)cc1 ; c1ccc(-c2cccc3c2oc2c(N(c4ccc(-c5ccc6c7ccc8ccccc8c7n(-c7ccccc7)c6c5)cc4)c4cc5ccccc5c5ccccc45)cccc23)cc1]

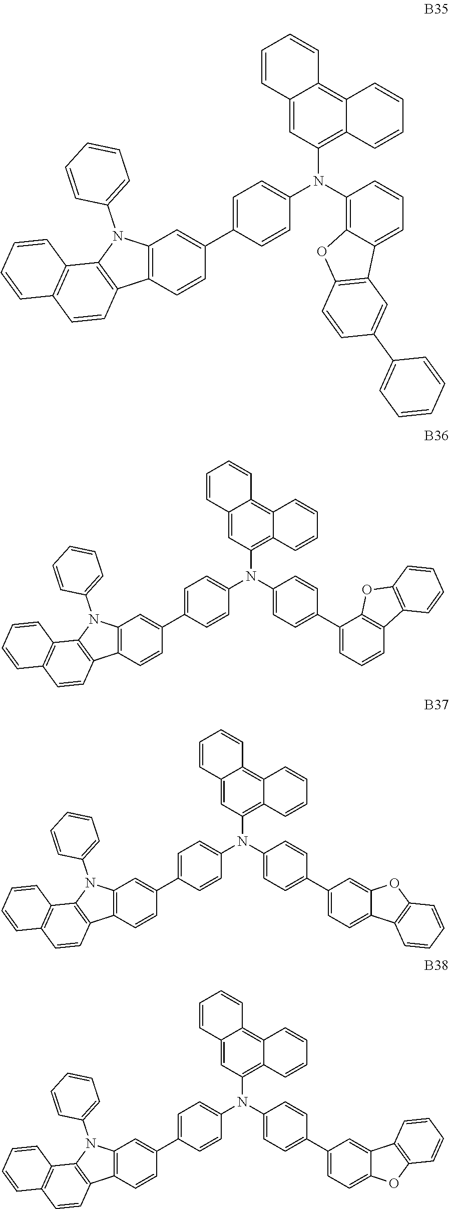 [SMILES: c1ccc(-c2ccc3oc4c(N(c5ccc(-c6ccc7c8ccc9ccccc9c8n(-c8ccccc8)c7c6)cc5)c5cc6ccccc6c6ccccc56)cccc4c3c2)cc1 ; c1ccc(-n2c3cc(-c4ccc(N(c5ccc(-c6cccc7c6oc6ccccc67)cc5)c5cc6ccccc6c6ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5cc6ccccc6c6ccccc56)cc4)ccc3c3ccc4ccccc4c32)cc1 ; c1ccc(-n2c3cc(-c4ccc(N(c5ccc(-c6ccc7oc8ccccc8c7c6)cc5)c5cc6ccccc6c6ccccc56)cc4)ccc3c3ccc4ccccc4c32)cc1]

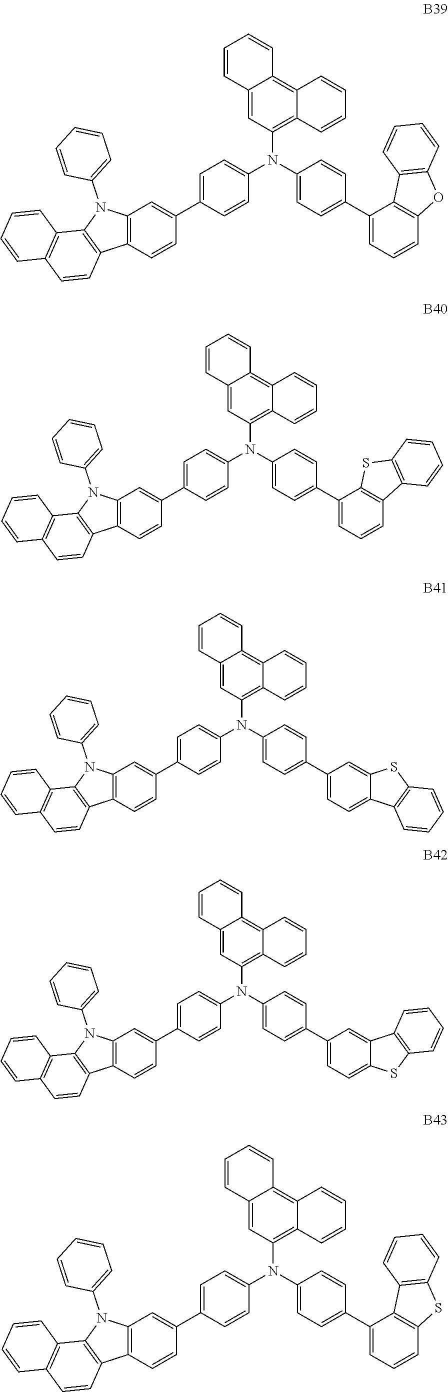

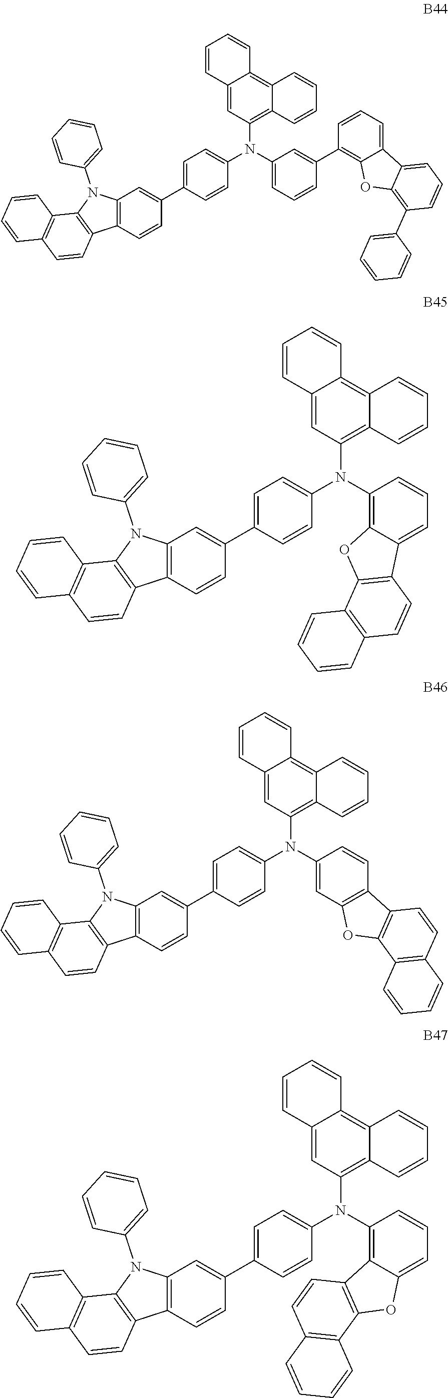 [SMILES: c1ccc(-c2cccc3c2oc2c(-c4cccc(N(c5ccc(-c6ccc7c8ccc9ccccc9c8n(-c8ccccc8)c7c6)cc5)c5cc6ccccc6c6ccccc56)c4)cccc23)cc1 ; c1ccc(-n2c3cc(-c4ccc(N(c5cc6ccccc6c6ccccc56)c5cccc6c5oc5c7ccccc7ccc65)cc4)ccc3c3ccc4ccccc4c32)cc1 ; c1ccc(-n2c3cc(-c4ccc(N(c5ccc6c(c5)oc5c7ccccc7ccc65)c5cc6ccccc6c6ccccc56)cc4)ccc3c3ccc4ccccc4c32)cc1 ; c1ccc(-n2c3cc(-c4ccc(N(c5cc6ccccc6c6ccccc56)c5cccc6oc7c8ccccc8ccc7c56)cc4)ccc3c3ccc4ccccc4c32)cc1]

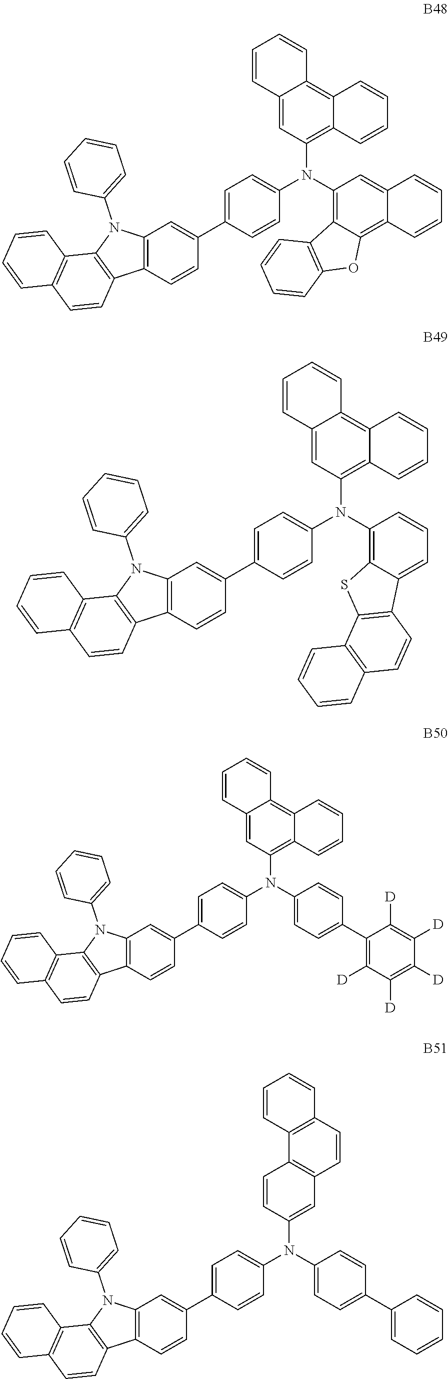 [SMILES: c1ccc(-n2c3cc(-c4ccc(N(c5cc6ccccc6c6ccccc56)c5cc6ccccc6c6oc7ccccc7c56)cc4)ccc3c3ccc4ccccc4c32)cc1 ; c1ccc(-n2c3cc(-c4ccc(N(c5cc6ccccc6c6ccccc56)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3cc4ccccc4c4ccccc34)cc2)c([2H])c1[2H] ; c1ccc(-c2ccc(N(c3ccc(-c4ccc5c6ccc7ccccc7c6n(-c6ccccc6)c5c4)cc3)c3ccc4c(ccc5ccccc54)c3)cc2)cc1]

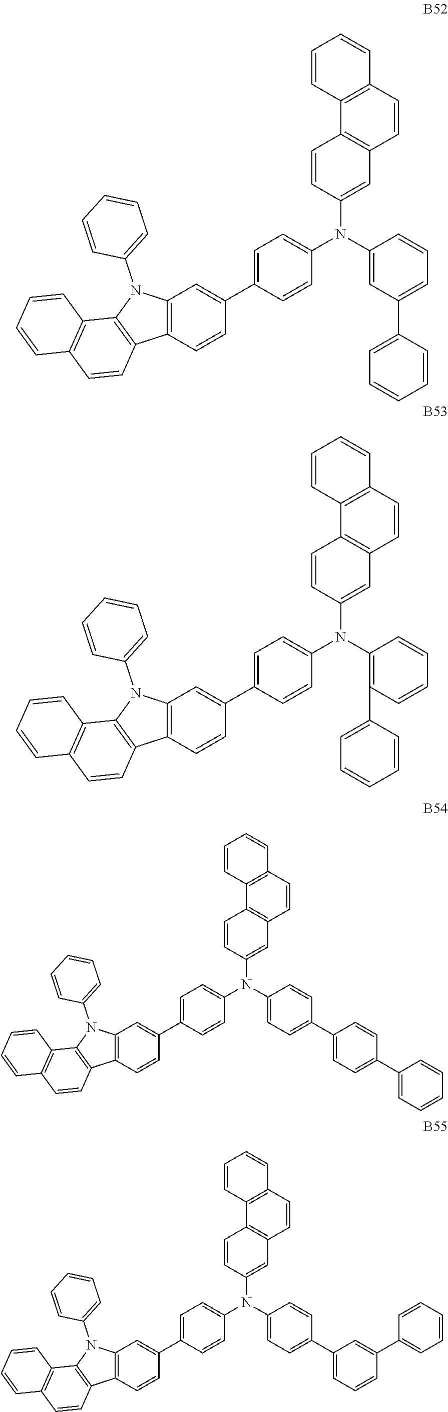 [SMILES: c1ccc(-c2cccc(N(c3ccc(-c4ccc5c6ccc7ccccc7c6n(-c6ccccc6)c5c4)cc3)c3ccc4c(ccc5ccccc54)c3)c2)cc1 ; c1ccc(-c2ccccc2N(c2ccc(-c3ccc4c5ccc6ccccc6c5n(-c5ccccc5)c4c3)cc2)c2ccc3c(ccc4ccccc43)c2)cc1 ; c1ccc(-c2ccc(-c3ccc(N(c4ccc(-c5ccc6c7ccc8ccccc8c7n(-c7ccccc7)c6c5)cc4)c4ccc5c(ccc6ccccc65)c4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(ccc6ccccc65)c4)cc3)c2)cc1]

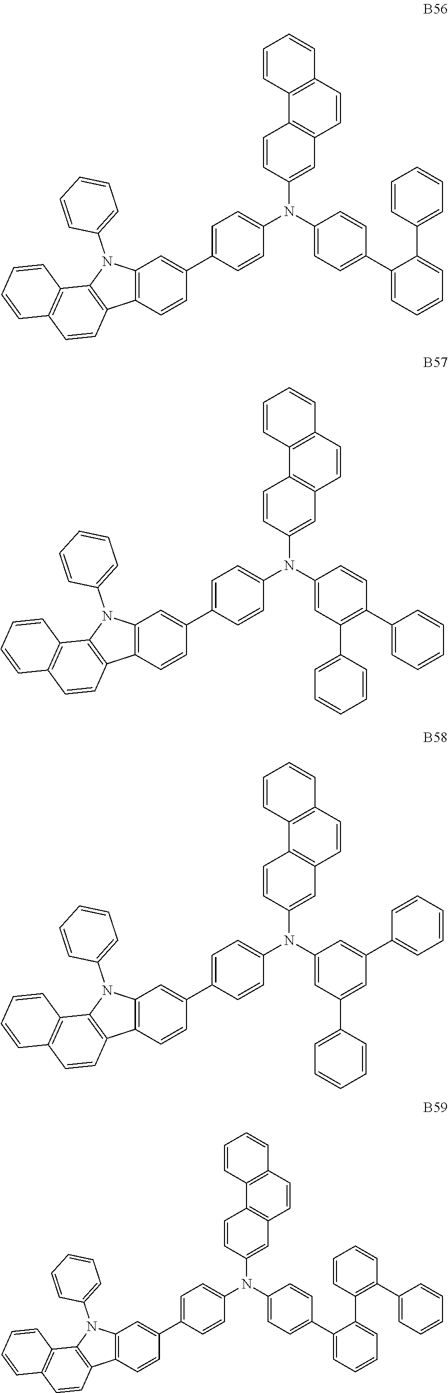 [SMILES: c1ccc(-c2ccccc2-c2ccc(N(c3ccc(-c4ccc5c6ccc7ccccc7c6n(-c6ccccc6)c5c4)cc3)c3ccc4c(ccc5ccccc54)c3)cc2)cc1 ; c1ccc(-c2ccc(N(c3ccc(-c4ccc5c6ccc7ccccc7c6n(-c6ccccc6)c5c4)cc3)c3ccc4c(ccc5ccccc54)c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(ccc5ccccc54)c3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(ccc5ccccc54)c3)cc2)cc1]

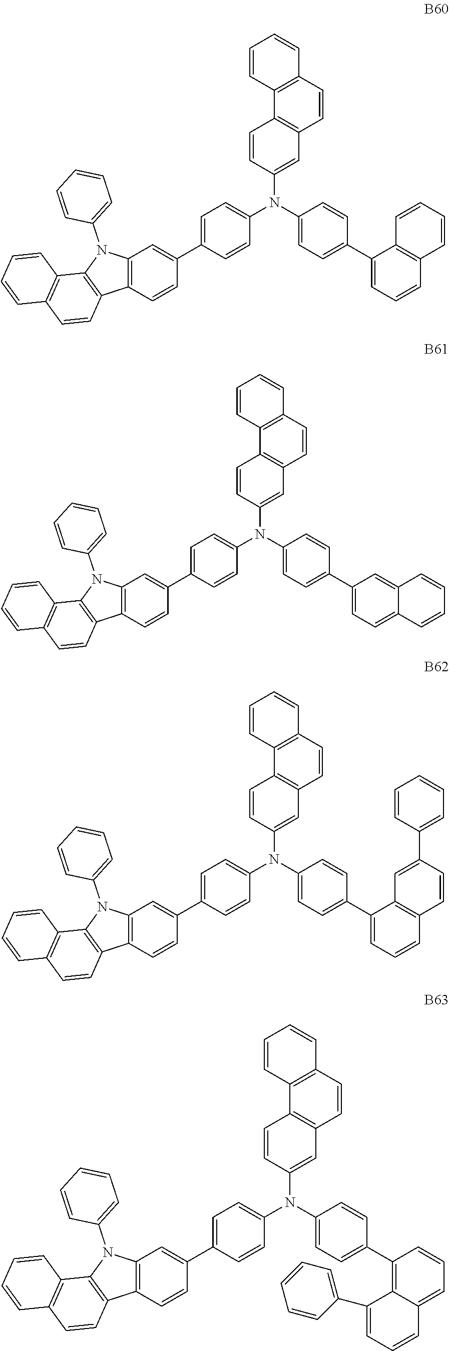 [SMILES: c1ccc(-n2c3cc(-c4ccc(N(c5ccc(-c6cccc7ccccc67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7ccccc7c6)cc5)c5ccc6c(ccc7ccccc76)c5)cc4)ccc3c3ccc4ccccc4c32)cc1 ; c1ccc(-c2ccc3cccc(-c4ccc(N(c5ccc(-c6ccc7c8ccc9ccccc9c8n(-c8ccccc8)c7c6)cc5)c5ccc6c(ccc7ccccc76)c5)cc4)c3c2)cc1 ; c1ccc(-c2cccc3cccc(-c4ccc(N(c5ccc(-c6ccc7c8ccc9ccccc9c8n(-c8ccccc8)c7c6)cc5)c5ccc6c(ccc7ccccc76)c5)cc4)c23)cc1]

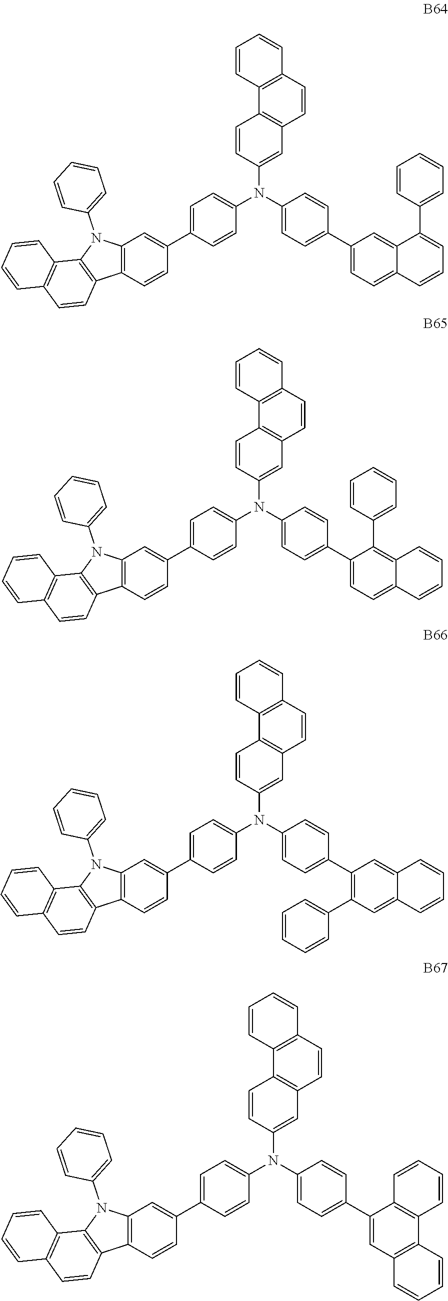 [SMILES: c1ccc(-c2cccc3ccc(-c4ccc(N(c5ccc(-c6ccc7c8ccc9ccccc9c8n(-c8ccccc8)c7c6)cc5)c5ccc6c(ccc7ccccc76)c5)cc4)cc23)cc1 ; c1ccc(-c2c(-c3ccc(N(c4ccc(-c5ccc6c7ccc8ccccc8c7n(-c7ccccc7)c6c5)cc4)c4ccc5c(ccc6ccccc65)c4)cc3)ccc3ccccc23)cc1 ; c1ccc(-c2cc3ccccc3cc2-c2ccc(N(c3ccc(-c4ccc5c6ccc7ccccc7c6n(-c6ccccc6)c5c4)cc3)c3ccc4c(ccc5ccccc54)c3)cc2)cc1 ; c1ccc(-n2c3cc(-c4ccc(N(c5ccc(-c6cc7ccccc7c7ccccc67)cc5)c5ccc6c(ccc7ccccc76)c5)cc4)ccc3c3ccc4ccccc4c32)cc1]

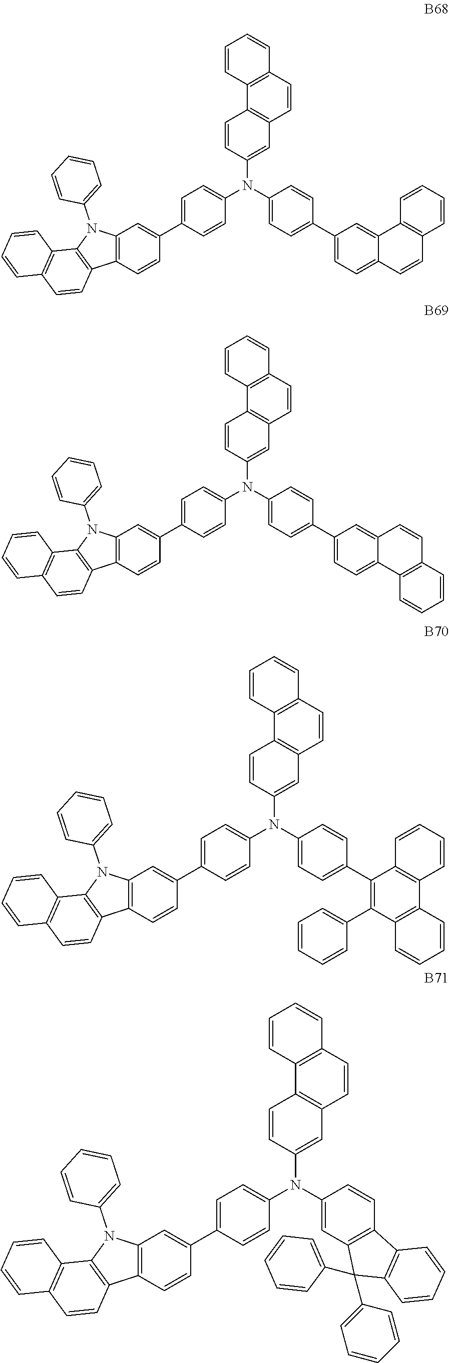 [SMILES: c1ccc(-n2c3cc(-c4ccc(N(c5ccc(-c6ccc7ccc8ccccc8c7c6)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6c(ccc7ccccc76)c5)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4ccc5c(ccc6ccccc65)c4)cc3)c3ccccc3c3ccccc23)cc1 ; c1ccc(-n2c3cc(-c4ccc(N(c5ccc6c(c5)C(c5ccccc5)(c5ccccc5)c5ccccc5-6)c5ccc6c(ccc7ccccc76)c5)cc4)ccc3c3ccc4ccccc4c32)cc1]

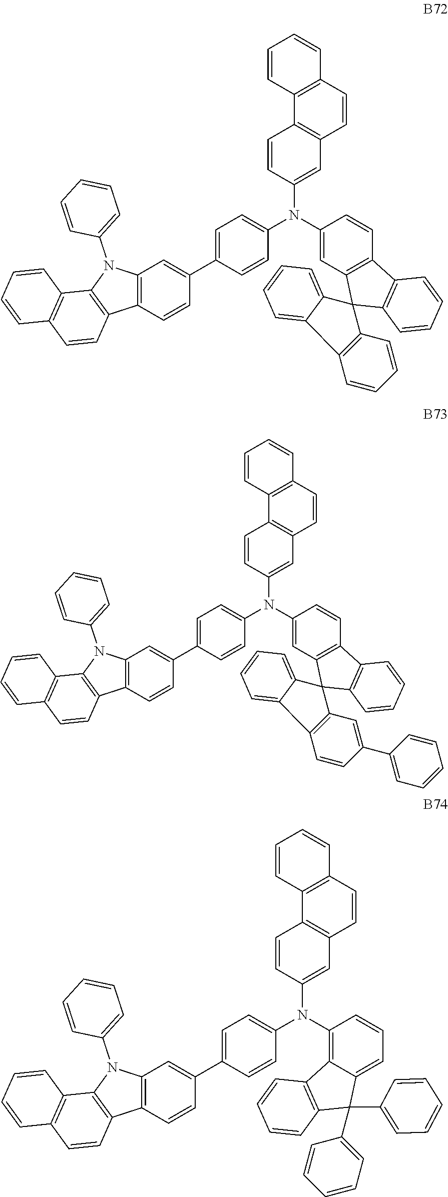 [SMILES: c1ccc(-n2c3cc(-c4ccc(N(c5ccc6c(c5)C5(c7ccccc7-c7ccccc75)c5ccccc5-6)c5ccc6c(ccc7ccccc76)c5)cc4)ccc3c3ccc4ccccc4c32)cc1 ; c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4ccc5c(ccc6ccccc65)c4)cc32)cc1 ; c1ccc(-n2c3cc(-c4ccc(N(c5ccc6c(ccc7ccccc76)c5)c5cccc6c5-c5ccccc5C6(c5ccccc5)c5ccccc5)cc4)ccc3c3ccc4ccccc4c32)cc1]

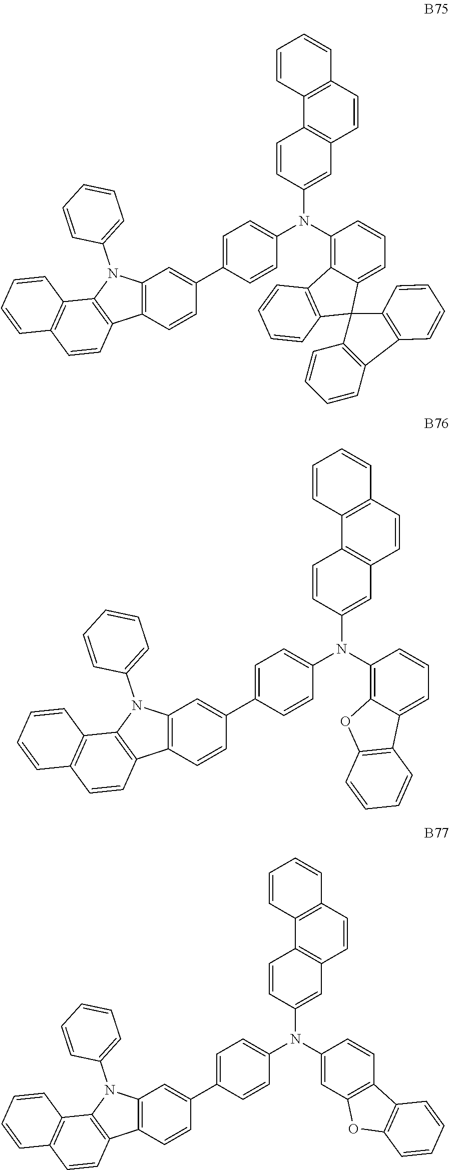 [SMILES: c1ccc(-n2c3cc(-c4ccc(N(c5ccc6c(ccc7ccccc76)c5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc6c(ccc7ccccc76)c5)c5cccc6c5oc5ccccc56)cc4)ccc3c3ccc4ccccc4c32)cc1 ; c1ccc(-n2c3cc(-c4ccc(N(c5ccc6c(ccc7ccccc76)c5)c5ccc6c(c5)oc5ccccc56)cc4)ccc3c3ccc4ccccc4c32)cc1]

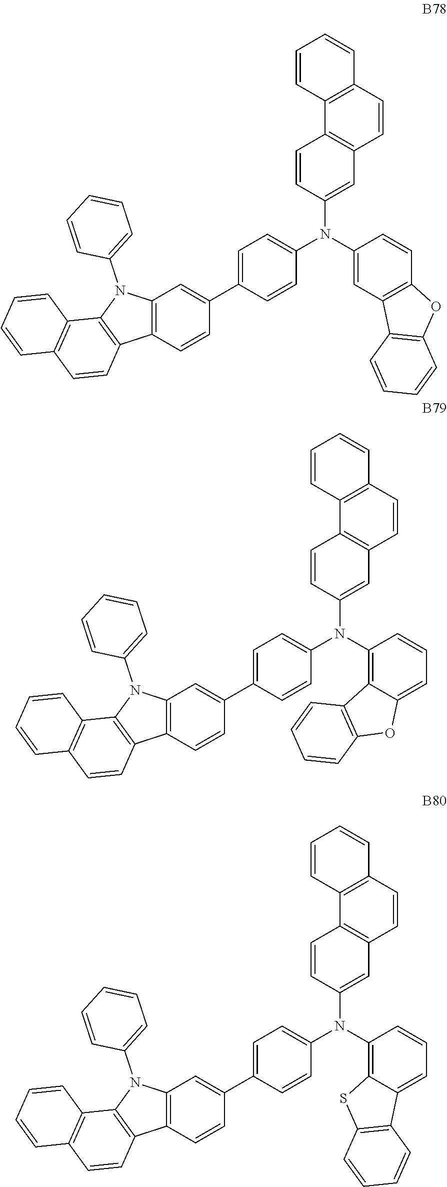 [SMILES: c1ccc(-n2c3cc(-c4ccc(N(c5ccc6c(ccc7ccccc76)c5)c5ccc6oc7ccccc7c6c5)cc4)ccc3c3ccc4ccccc4c32)cc1 ; c1ccc(-n2c3cc(-c4ccc(N(c5ccc6c(ccc7ccccc76)c5)c5cccc6oc7ccccc7c56)cc4)ccc3c3ccc4ccccc4c32)cc1 ; c1ccc(-n2c3cc(-c4ccc(N(c5ccc6c(ccc7ccccc76)c5)c5cccc6c5sc5ccccc56)cc4)ccc3c3ccc4ccccc4c32)cc1]

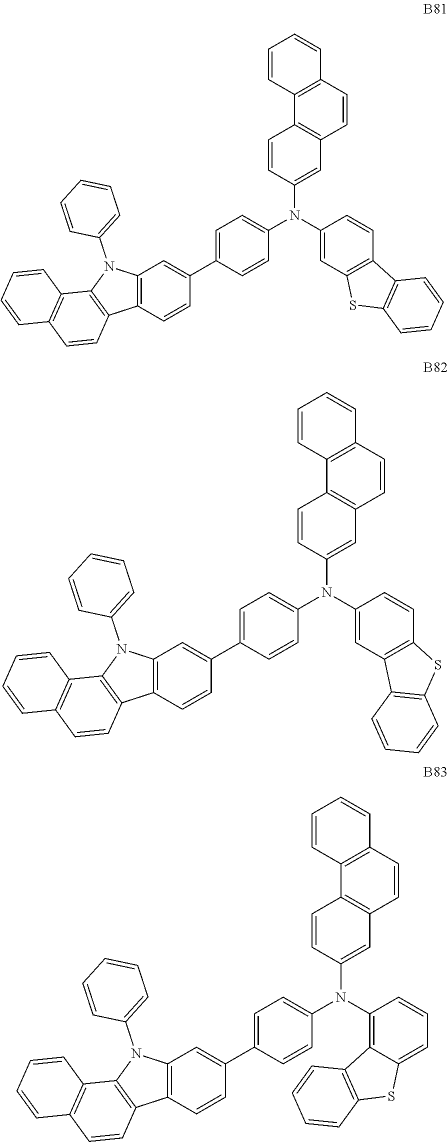 [SMILES: c1ccc(-n2c3cc(-c4ccc(N(c5ccc6c(ccc7ccccc76)c5)c5ccc6c(c5)sc5ccccc56)cc4)ccc3c3ccc4ccccc4c32)cc1 ; c1ccc(-n2c3cc(-c4ccc(N(c5ccc6c(ccc7ccccc76)c5)c5ccc6sc7ccccc7c6c5)cc4)ccc3c3ccc4ccccc4c32)cc1 ; c1ccc(-n2c3cc(-c4ccc(N(c5ccc6c(ccc7ccccc76)c5)c5cccc6sc7ccccc7c56)cc4)ccc3c3ccc4ccccc4c32)cc1]

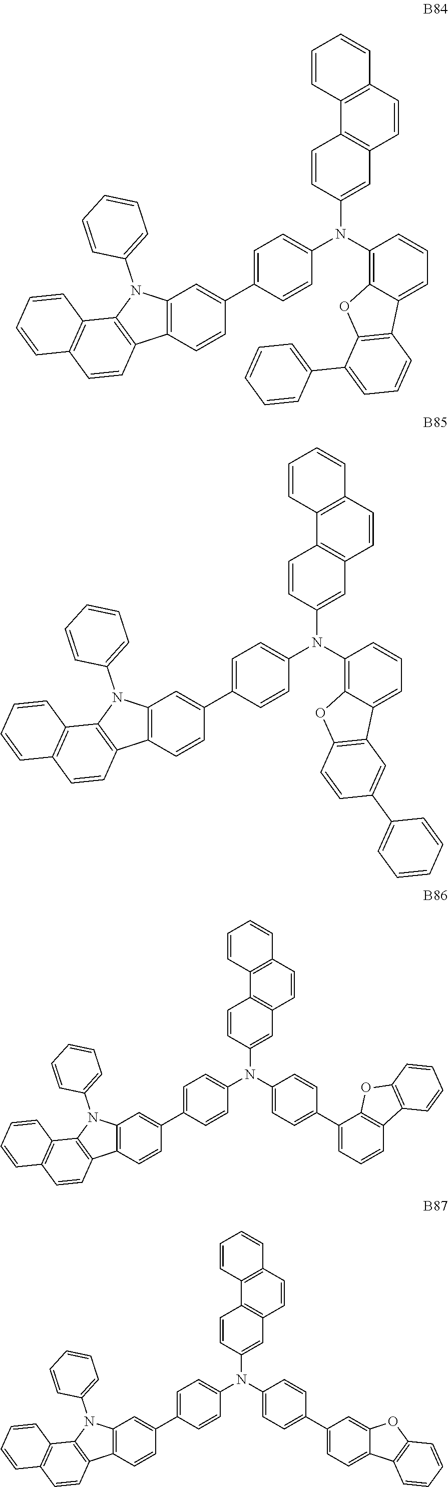 [SMILES: c1ccc(-c2cccc3c2oc2c(N(c4ccc(-c5ccc6c7ccc8ccccc8c7n(-c7ccccc7)c6c5)cc4)c4ccc5c(ccc6ccccc65)c4)cccc23)cc1 ; c1ccc(-c2ccc3oc4c(N(c5ccc(-c6ccc7c8ccc9ccccc9c8n(-c8ccccc8)c7c6)cc5)c5ccc6c(ccc7ccccc76)c5)cccc4c3c2)cc1 ; c1ccc(-n2c3cc(-c4ccc(N(c5ccc(-c6cccc7c6oc6ccccc67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7c(c6)oc6ccccc67)cc5)c5ccc6c(ccc7ccccc76)c5)cc4)ccc3c3ccc4ccccc4c32)cc1]

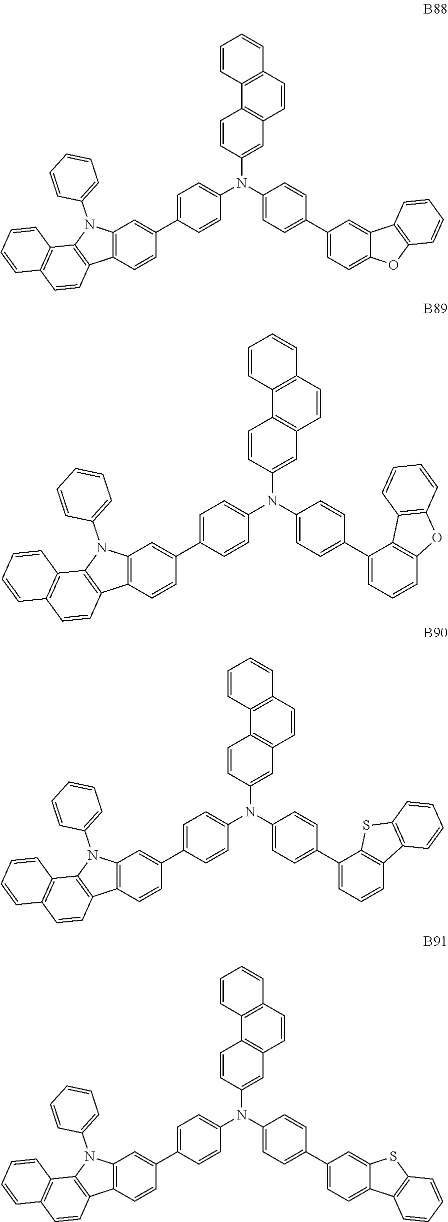 [SMILES: c1ccc(-n2c3cc(-c4ccc(N(c5ccc(-c6ccc7oc8ccccc8c7c6)cc5)c5ccc6c(ccc7ccccc76)c5)cc4)ccc3c3ccc4ccccc4c32)cc1 ; c1ccc(-n2c3cc(-c4ccc(N(c5ccc(-c6cccc7oc8ccccc8c67)cc5)c5ccc6c(ccc7ccccc76)c5)cc4)ccc3c3ccc4ccccc4c32)cc1 ; c1ccc(-n2c3cc(-c4ccc(N(c5ccc(-c6cccc7c6sc6ccccc67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7c(c6)sc6ccccc67)cc5)c5ccc6c(ccc7ccccc76)c5)cc4)ccc3c3ccc4ccccc4c32)cc1]

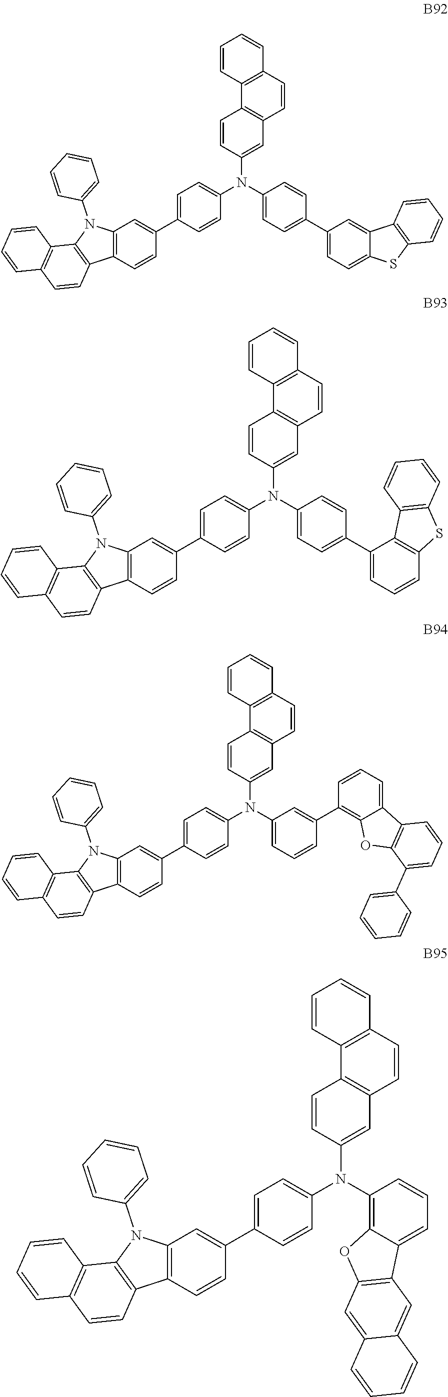 [SMILES: c1ccc(-n2c3cc(-c4ccc(N(c5ccc(-c6ccc7sc8ccccc8c7c6)cc5)c5ccc6c(ccc7ccccc76)c5)cc4)ccc3c3ccc4ccccc4c32)cc1 ; c1ccc(-n2c3cc(-c4ccc(N(c5ccc(-c6cccc7sc8ccccc8c67)cc5)c5ccc6c(ccc7ccccc76)c5)cc4)ccc3c3ccc4ccccc4c32)cc1 ; c1ccc(-c2cccc3c2oc2c(-c4cccc(N(c5ccc(-c6ccc7c8ccc9ccccc9c8n(-c8ccccc8)c7c6)cc5)c5ccc6c(ccc7ccccc76)c5)c4)cccc23)cc1 ; c1ccc(-n2c3cc(-c4ccc(N(c5ccc6c(ccc7ccccc76)c5)c5cccc6c5oc5cc7ccccc7cc56)cc4)ccc3c3ccc4ccccc4c32)cc1]

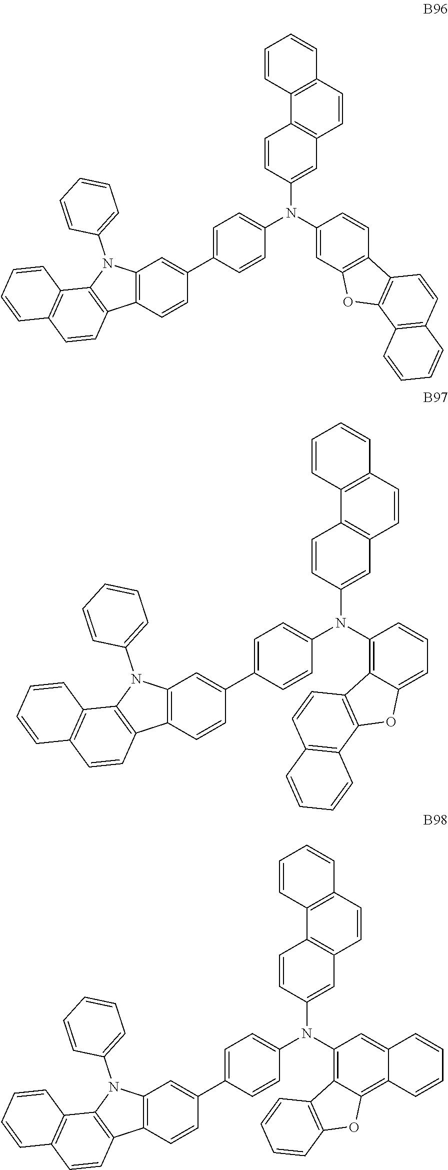

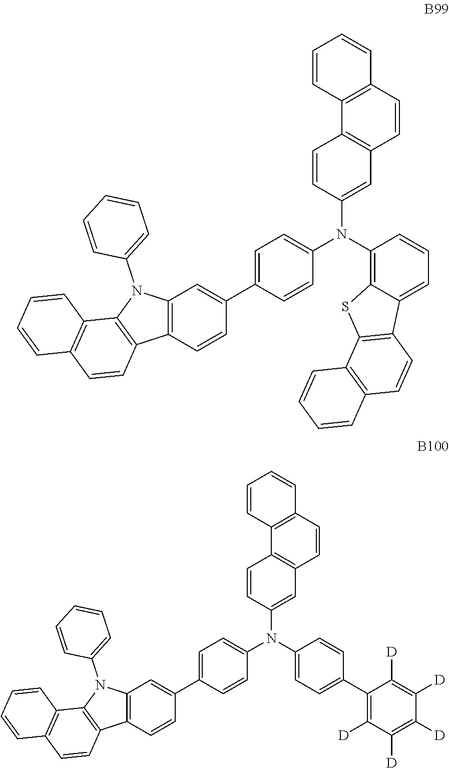 [SMILES: c1ccc(-n2c3cc(-c4ccc(N(c5ccc6c(ccc7ccccc76)c5)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4c(ccc5ccccc54)c3)cc2)c([2H])c1[2H]]

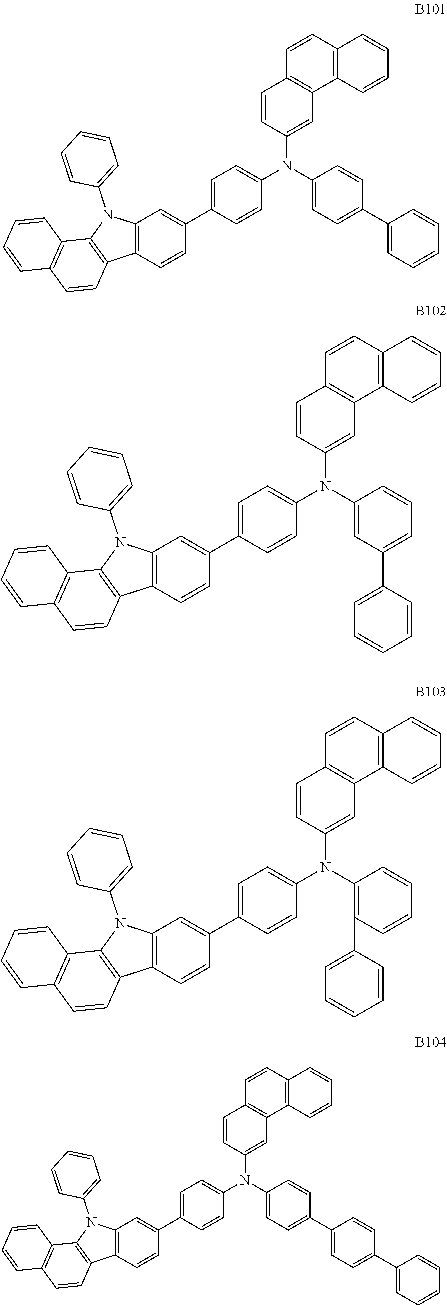 [SMILES: c1ccc(-c2ccc(N(c3ccc(-c4ccc5c6ccc7ccccc7c6n(-c6ccccc6)c5c4)cc3)c3ccc4ccc5ccccc5c4c3)cc2)cc1 ; c1ccc(-c2cccc(N(c3ccc(-c4ccc5c6ccc7ccccc7c6n(-c6ccccc6)c5c4)cc3)c3ccc4ccc5ccccc5c4c3)c2)cc1 ; c1ccc(-c2ccccc2N(c2ccc(-c3ccc4c5ccc6ccccc6c5n(-c5ccccc5)c4c3)cc2)c2ccc3ccc4ccccc4c3c2)cc1 ; c1ccc(-c2ccc(-c3ccc(N(c4ccc(-c5ccc6c7ccc8ccccc8c7n(-c7ccccc7)c6c5)cc4)c4ccc5ccc6ccccc6c5c4)cc3)cc2)cc1]

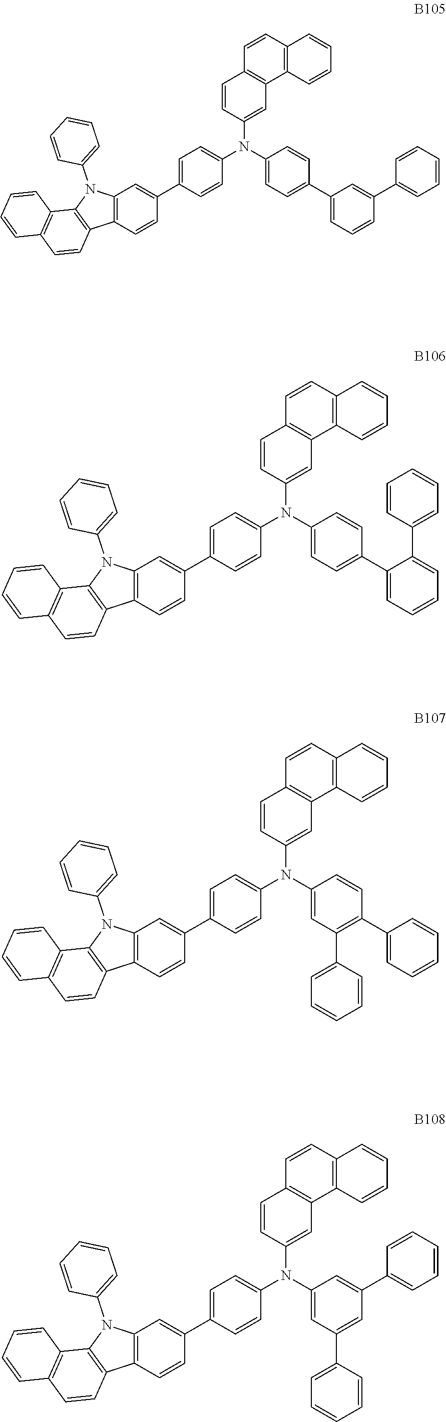 [SMILES: c1ccc(-c2cccc(-c3ccc(N(c4ccc(-c5ccc6c7ccc8ccccc8c7n(-c7ccccc7)c6c5)cc4)c4ccc5ccc6ccccc6c5c4)cc3)c2)cc1 ; c1ccc(-c2ccccc2-c2ccc(N(c3ccc(-c4ccc5c6ccc7ccccc7c6n(-c6ccccc6)c5c4)cc3)c3ccc4ccc5ccccc5c4c3)cc2)cc1 ; c1ccc(-c2ccc(N(c3ccc(-c4ccc5c6ccc7ccccc7c6n(-c6ccccc6)c5c4)cc3)c3ccc4ccc5ccccc5c4c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4ccc5ccccc5c4c3)c2)cc1]

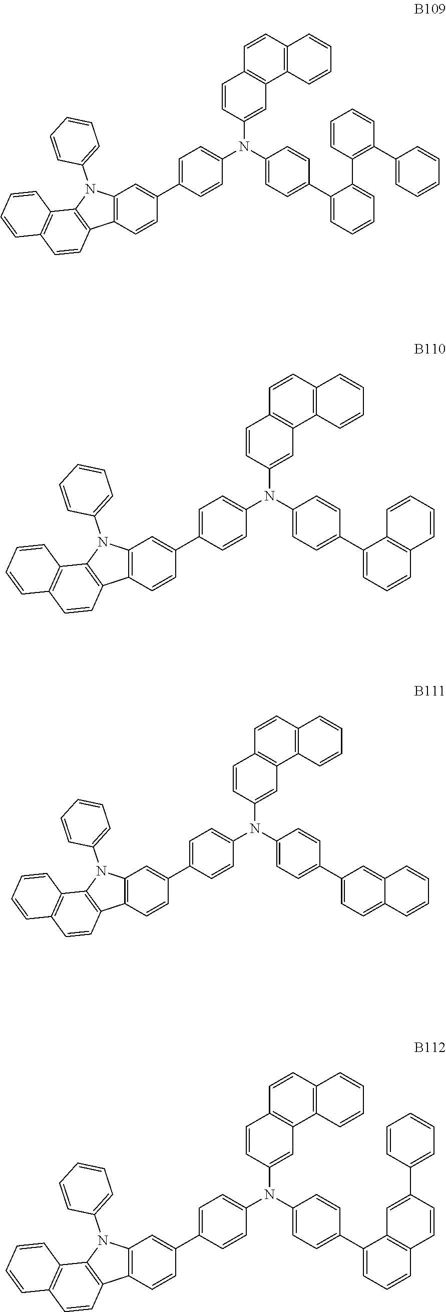 [SMILES: c1ccc(-c2ccccc2-c2ccccc2-c2ccc(N(c3ccc(-c4ccc5c6ccc7ccccc7c6n(-c6ccccc6)c5c4)cc3)c3ccc4ccc5ccccc5c4c3)cc2)cc1 ; c1ccc(-n2c3cc(-c4ccc(N(c5ccc(-c6cccc7ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7ccccc7c6)cc5)c5ccc6ccc7ccccc7c6c5)cc4)ccc3c3ccc4ccccc4c32)cc1 ; c1ccc(-c2ccc3cccc(-c4ccc(N(c5ccc(-c6ccc7c8ccc9ccccc9c8n(-c8ccccc8)c7c6)cc5)c5ccc6ccc7ccccc7c6c5)cc4)c3c2)cc1]

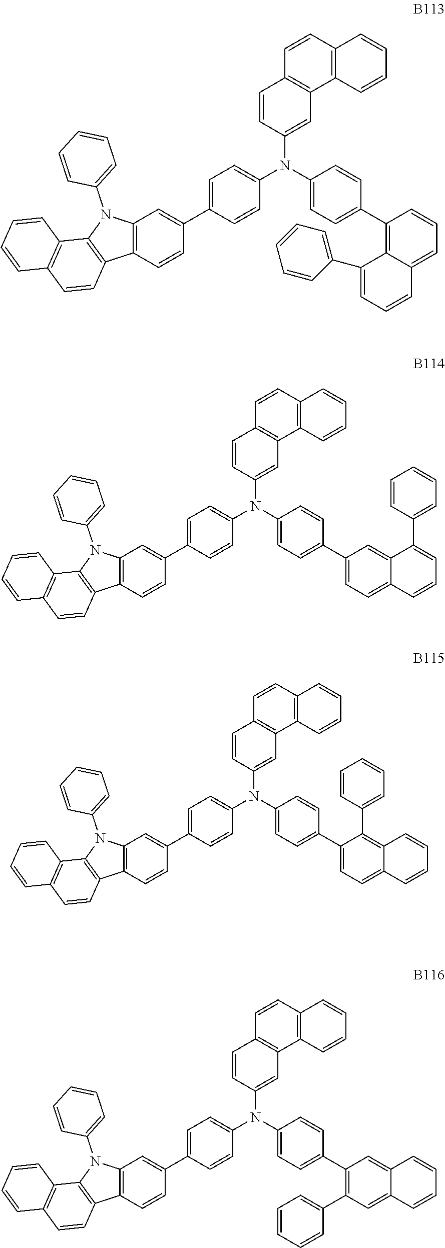 [SMILES: c1ccc(-c2cccc3cccc(-c4ccc(N(c5ccc(-c6ccc7c8ccc9ccccc9c8n(-c8ccccc8)c7c6)cc5)c5ccc6ccc7ccccc7c6c5)cc4)c23)cc1 ; c1ccc(-c2cccc3ccc(-c4ccc(N(c5ccc(-c6ccc7c8ccc9ccccc9c8n(-c8ccccc8)c7c6)cc5)c5ccc6ccc7ccccc7c6c5)cc4)cc23)cc1 ; c1ccc(-c2c(-c3ccc(N(c4ccc(-c5ccc6c7ccc8ccccc8c7n(-c7ccccc7)c6c5)cc4)c4ccc5ccc6ccccc6c5c4)cc3)ccc3ccccc23)cc1 ; c1ccc(-c2cc3ccccc3cc2-c2ccc(N(c3ccc(-c4ccc5c6ccc7ccccc7c6n(-c6ccccc6)c5c4)cc3)c3ccc4ccc5ccccc5c4c3)cc2)cc1]

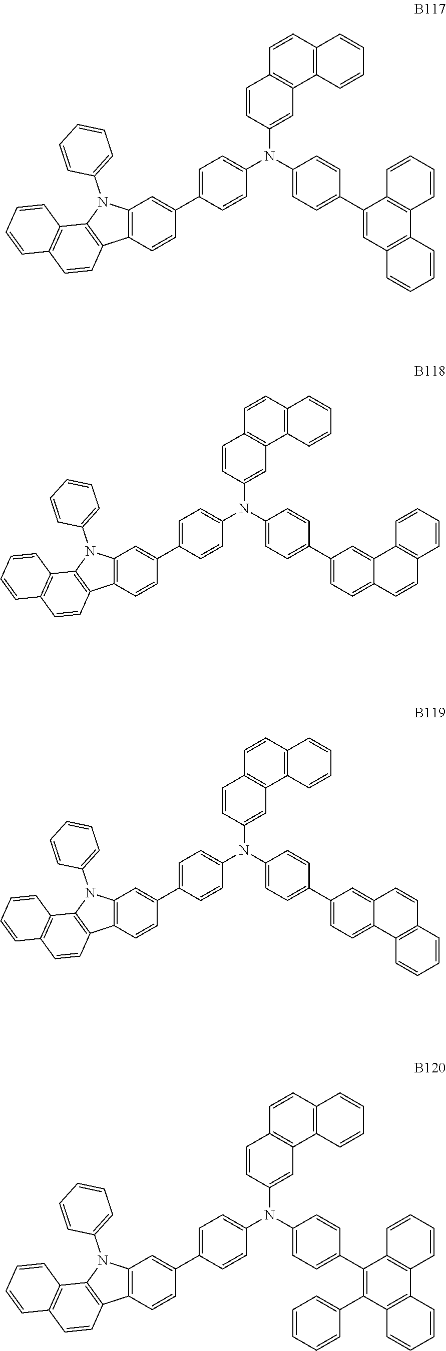 [SMILES: c1ccc(-n2c3cc(-c4ccc(N(c5ccc(-c6cc7ccccc7c7ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7ccc8ccccc8c7c6)cc5)c5ccc6ccc7ccccc7c6c5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6ccc7ccccc7c6c5)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4ccc5ccc6ccccc6c5c4)cc3)c3ccccc3c3ccccc23)cc1]

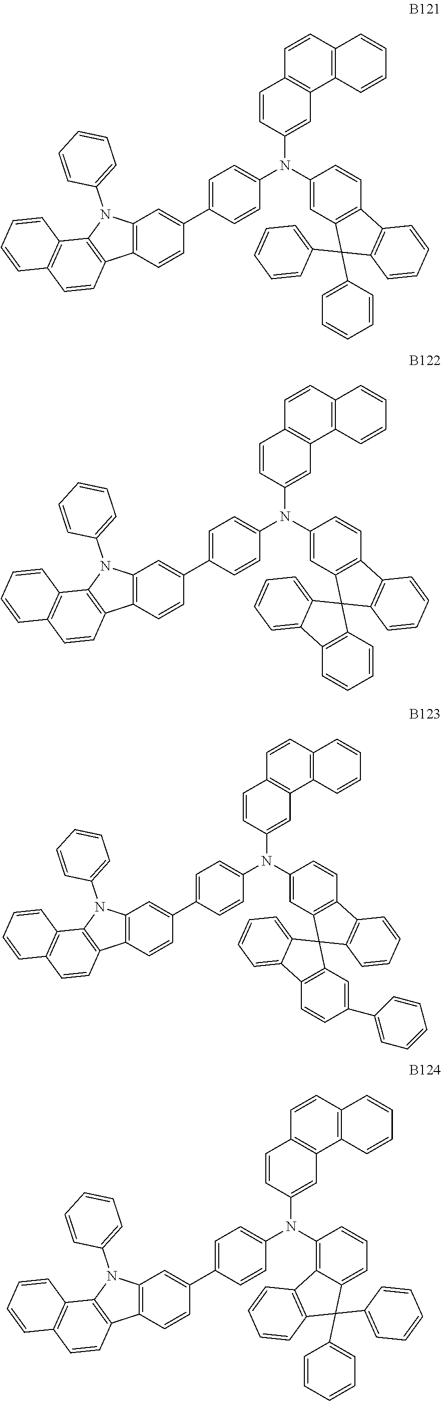 [SMILES: c1ccc(-n2c3cc(-c4ccc(N(c5ccc6c(c5)C(c5ccccc5)(c5ccccc5)c5ccccc5-6)c5ccc6ccc7ccccc7c6c5)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6ccc7ccccc7c6c5)cc4)ccc3c3ccc4ccccc4c32)cc1 ; c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4ccc5ccc6ccccc6c5c4)cc32)cc1 ; c1ccc(-n2c3cc(-c4ccc(N(c5ccc6ccc7ccccc7c6c5)c5cccc6c5-c5ccccc5C6(c5ccccc5)c5ccccc5)cc4)ccc3c3ccc4ccccc4c32)cc1]

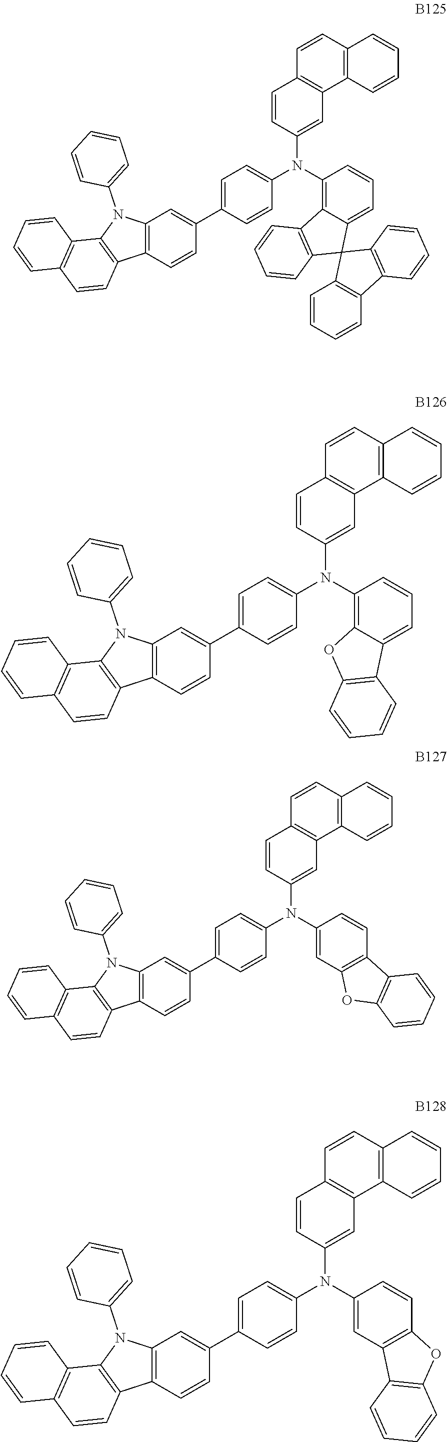 [SMILES: c1ccc(-n2c3cc(-c4ccc(N(c5ccc6ccc7ccccc7c6c5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc6ccc7ccccc7c6c5)c5cccc6c5oc5ccccc56)cc4)ccc3c3ccc4ccccc4c32)cc1 ; c1ccc(-n2c3cc(-c4ccc(N(c5ccc6c(c5)oc5ccccc56)c5ccc6ccc7ccccc7c6c5)cc4)ccc3c3ccc4ccccc4c32)cc1 ; c1ccc(-n2c3cc(-c4ccc(N(c5ccc6ccc7ccccc7c6c5)c5ccc6oc7ccccc7c6c5)cc4)ccc3c3ccc4ccccc4c32)cc1]

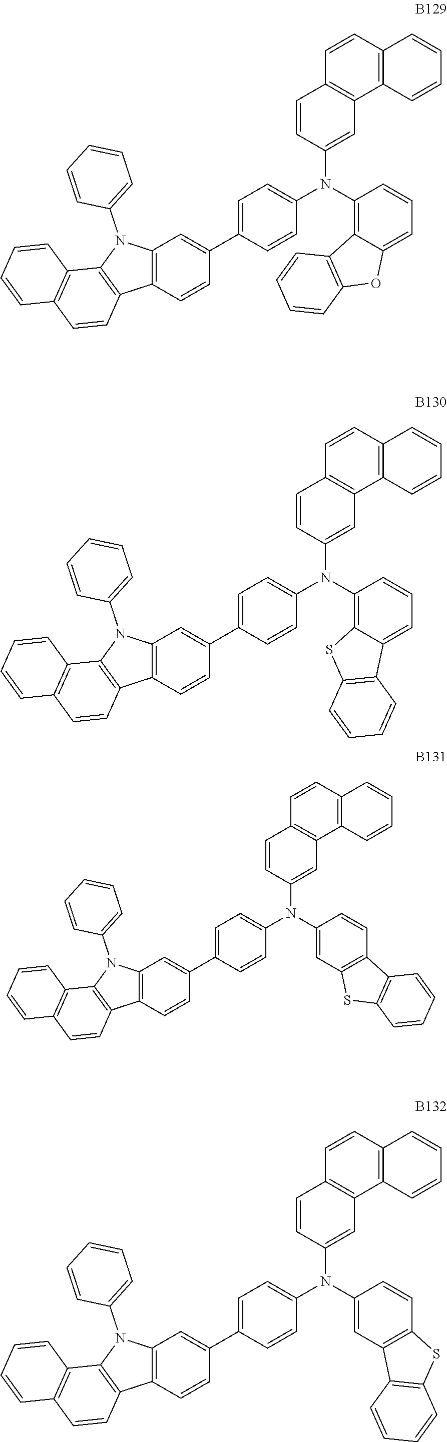 [SMILES: c1ccc(-n2c3cc(-c4ccc(N(c5ccc6ccc7ccccc7c6c5)c5cccc6oc7ccccc7c56)cc4)ccc3c3ccc4ccccc4c32)cc1 ; c1ccc(-n2c3cc(-c4ccc(N(c5ccc6ccc7ccccc7c6c5)c5cccc6c5sc5ccccc56)cc4)ccc3c3ccc4ccccc4c32)cc1 ; c1ccc(-n2c3cc(-c4ccc(N(c5ccc6c(c5)sc5ccccc56)c5ccc6ccc7ccccc7c6c5)cc4)ccc3c3ccc4ccccc4c32)cc1 ; c1ccc(-n2c3cc(-c4ccc(N(c5ccc6ccc7ccccc7c6c5)c5ccc6sc7ccccc7c6c5)cc4)ccc3c3ccc4ccccc4c32)cc1]

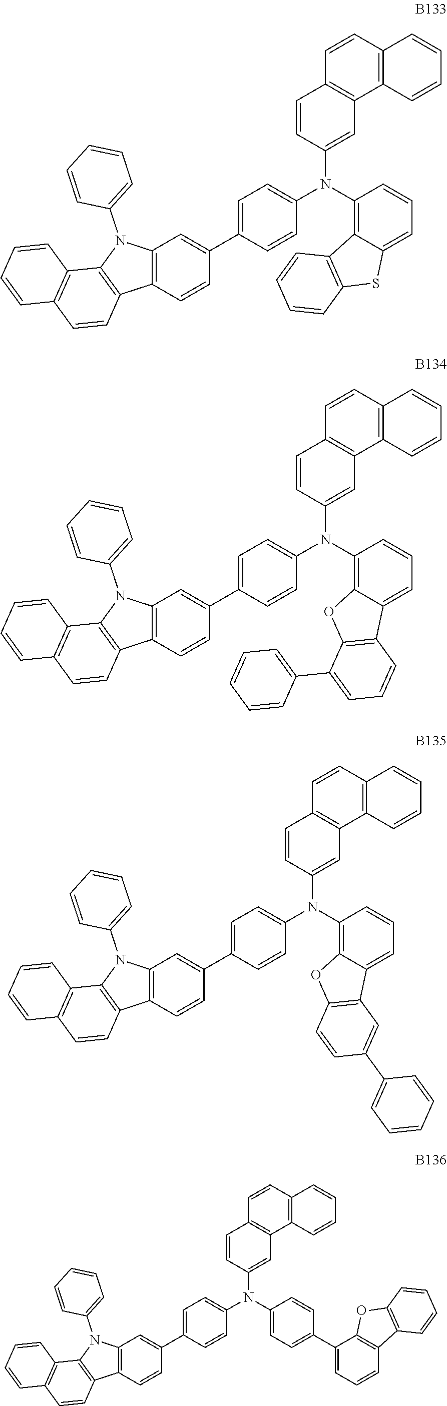 [SMILES: c1ccc(-n2c3cc(-c4ccc(N(c5ccc6ccc7ccccc7c6c5)c5cccc6sc7ccccc7c56)cc4)ccc3c3ccc4ccccc4c32)cc1 ; c1ccc(-c2cccc3c2oc2c(N(c4ccc(-c5ccc6c7ccc8ccccc8c7n(-c7ccccc7)c6c5)cc4)c4ccc5ccc6ccccc6c5c4)cccc23)cc1 ; c1ccc(-c2ccc3oc4c(N(c5ccc(-c6ccc7c8ccc9ccccc9c8n(-c8ccccc8)c7c6)cc5)c5ccc6ccc7ccccc7c6c5)cccc4c3c2)cc1 ; c1ccc(-n2c3cc(-c4ccc(N(c5ccc(-c6cccc7c6oc6ccccc67)cc5)c5ccc6ccc7ccccc7c6c5)cc4)ccc3c3ccc4ccccc4c32)cc1]

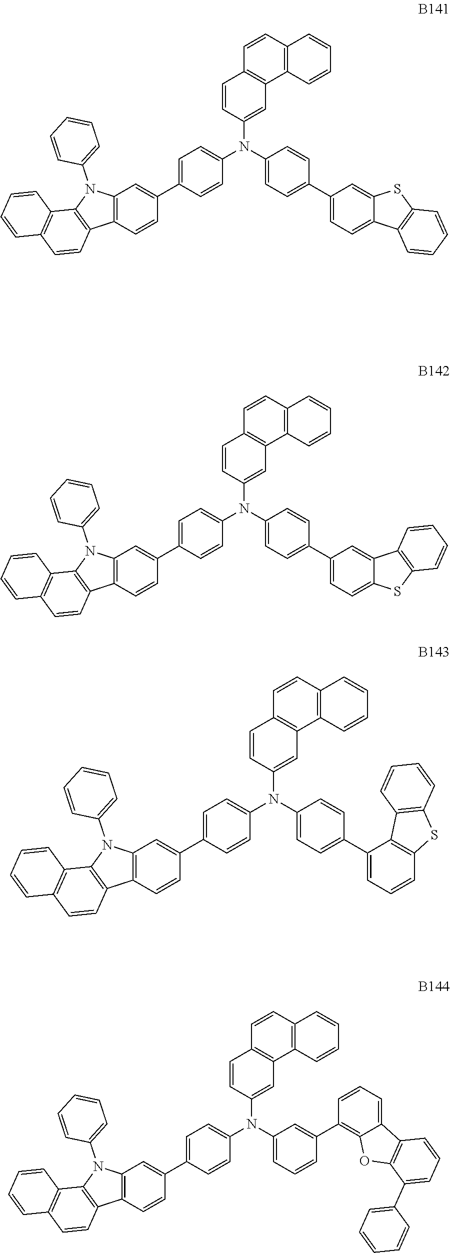 [SMILES: c1ccc(-n2c3cc(-c4ccc(N(c5ccc(-c6ccc7c(c6)sc6ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7sc8ccccc8c7c6)cc5)c5ccc6ccc7ccccc7c6c5)cc4)ccc3c3ccc4ccccc4c32)cc1 ; c1ccc(-n2c3cc(-c4ccc(N(c5ccc(-c6cccc7sc8ccccc8c67)cc5)c5ccc6ccc7ccccc7c6c5)cc4)ccc3c3ccc4ccccc4c32)cc1 ; c1ccc(-c2cccc3c2oc2c(-c4cccc(N(c5ccc(-c6ccc7c8ccc9ccccc9c8n(-c8ccccc8)c7c6)cc5)c5ccc6ccc7ccccc7c6c5)c4)cccc23)cc1]

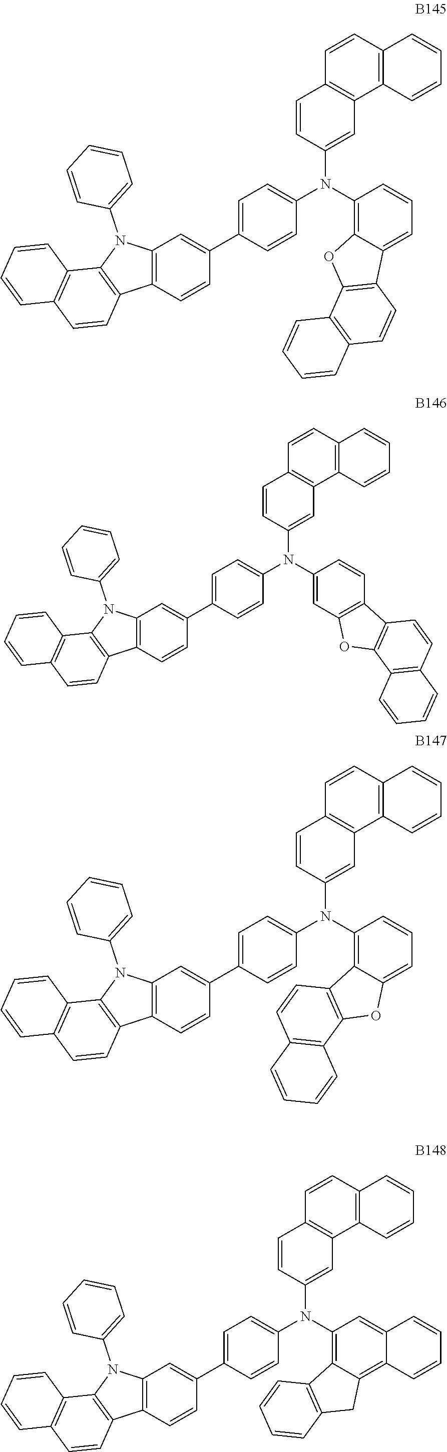 [SMILES: c1ccc(-n2c3cc(-c4ccc(N(c5ccc6ccc7ccccc7c6c5)c5cccc6c5oc5c7ccccc7ccc65)cc4)ccc3c3ccc4ccccc4c32)cc1 ; c1ccc(-n2c3cc(-c4ccc(N(c5ccc6c(c5)oc5c7ccccc7ccc65)c5ccc6ccc7ccccc7c6c5)cc4)ccc3c3ccc4ccccc4c32)cc1 ; c1ccc(-n2c3cc(-c4ccc(N(c5ccc6ccc7ccccc7c6c5)c5cccc6oc7c8ccccc8ccc7c56)cc4)ccc3c3ccc4ccccc4c32)cc1 ; c1ccc(-n2c3cc(-c4ccc(N(c5ccc6ccc7ccccc7c6c5)c5cc6ccccc6c6c5-c5ccccc5C6)cc4)ccc3c3ccc4ccccc4c32)cc1]

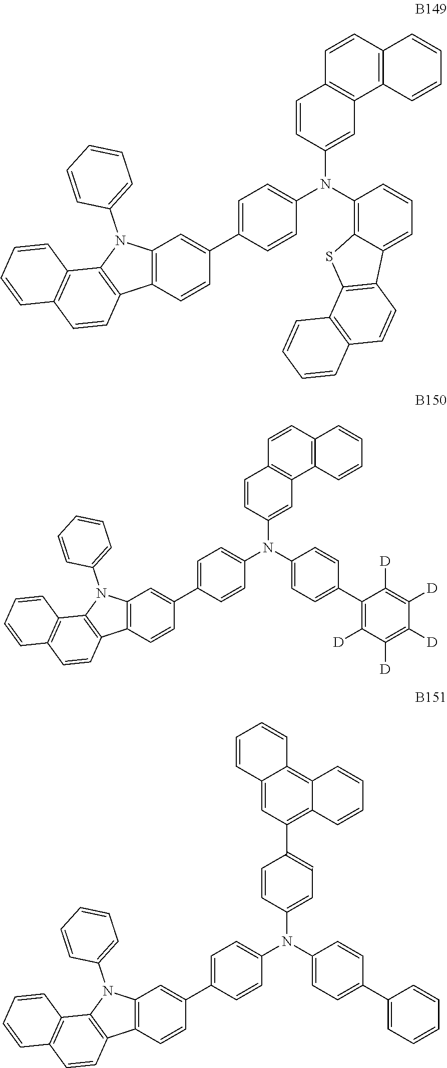 [SMILES: c1ccc(-n2c3cc(-c4ccc(N(c5ccc6ccc7ccccc7c6c5)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4ccc5ccccc5c4c3)cc2)c([2H])c1[2H] ; c1ccc(-c2ccc(N(c3ccc(-c4ccc5c6ccc7ccccc7c6n(-c6ccccc6)c5c4)cc3)c3ccc(-c4cc5ccccc5c5ccccc45)cc3)cc2)cc1]

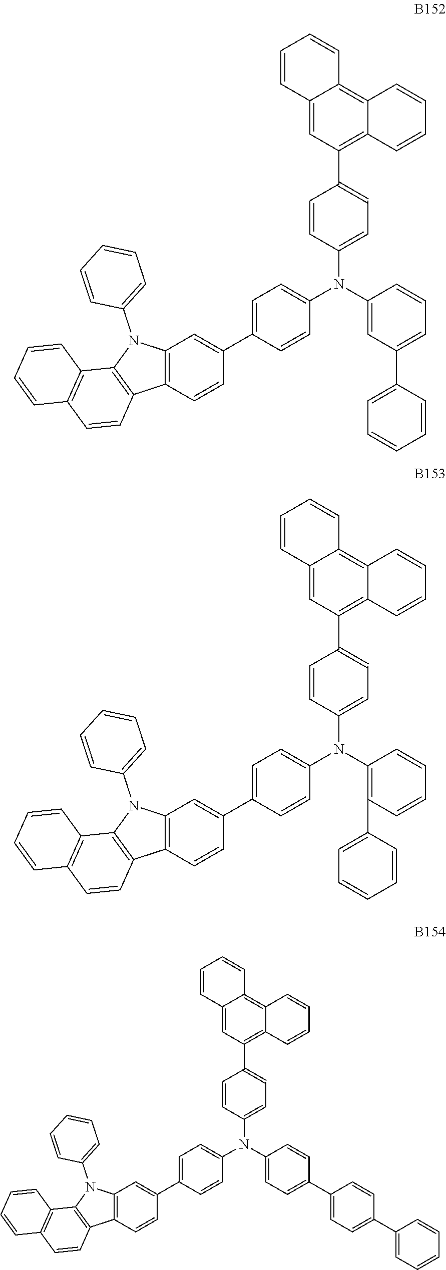 [SMILES: c1ccc(-c2cccc(N(c3ccc(-c4ccc5c6ccc7ccccc7c6n(-c6ccccc6)c5c4)cc3)c3ccc(-c4cc5ccccc5c5ccccc45)cc3)c2)cc1 ; c1ccc(-c2ccccc2N(c2ccc(-c3ccc4c5ccc6ccccc6c5n(-c5ccccc5)c4c3)cc2)c2ccc(-c3cc4ccccc4c4ccccc34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cc6ccccc6c6ccccc56)cc4)cc3)cc2)cc1]

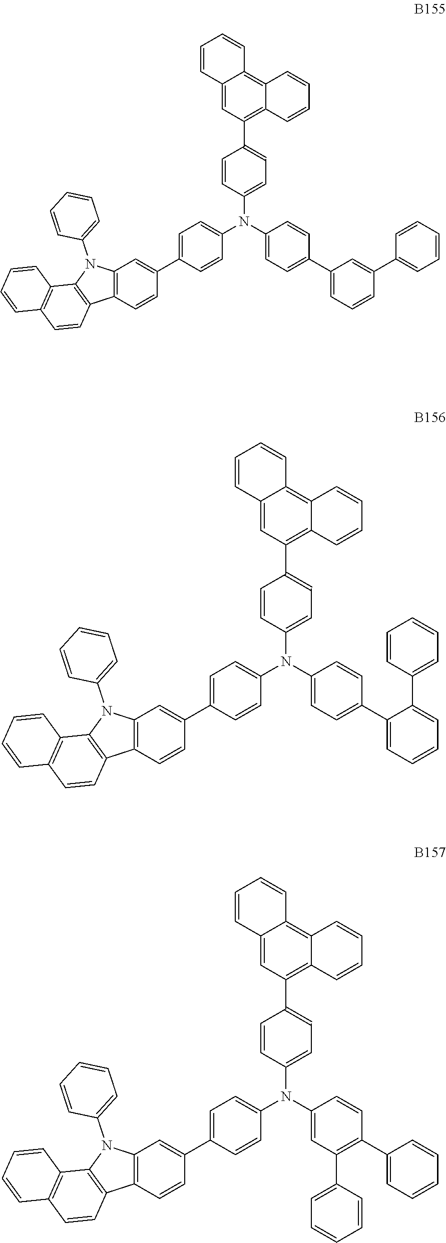 [SMILES: c1ccc(-c2cccc(-c3ccc(N(c4ccc(-c5ccc6c7ccc8ccccc8c7n(-c7ccccc7)c6c5)cc4)c4ccc(-c5cc6ccccc6c6ccccc56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cc5ccccc5c5ccccc45)cc3)cc2)cc1 ; c1ccc(-c2ccc(N(c3ccc(-c4ccc5c6ccc7ccccc7c6n(-c6ccccc6)c5c4)cc3)c3ccc(-c4cc5ccccc5c5ccccc45)cc3)cc2-c2ccccc2)cc1]

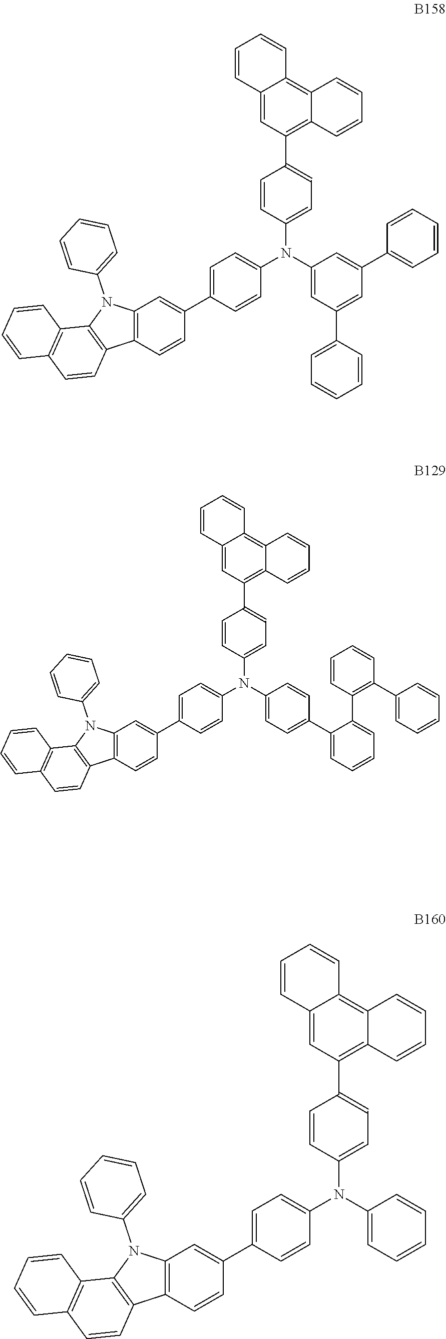 [SMILES: c1ccc(-c2cc(-c3ccccc3)cc(N(c3ccc(-c4ccc5c6ccc7ccccc7c6n(-c6ccccc6)c5c4)cc3)c3ccc(-c4cc5ccccc5c5ccccc45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cc5ccccc5c5ccccc45)cc3)cc2)cc1 ; c1ccc(N(c2ccc(-c3ccc4c5ccc6ccccc6c5n(-c5ccccc5)c4c3)cc2)c2ccc(-c3cc4ccccc4c4ccccc34)cc2)cc1]

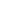

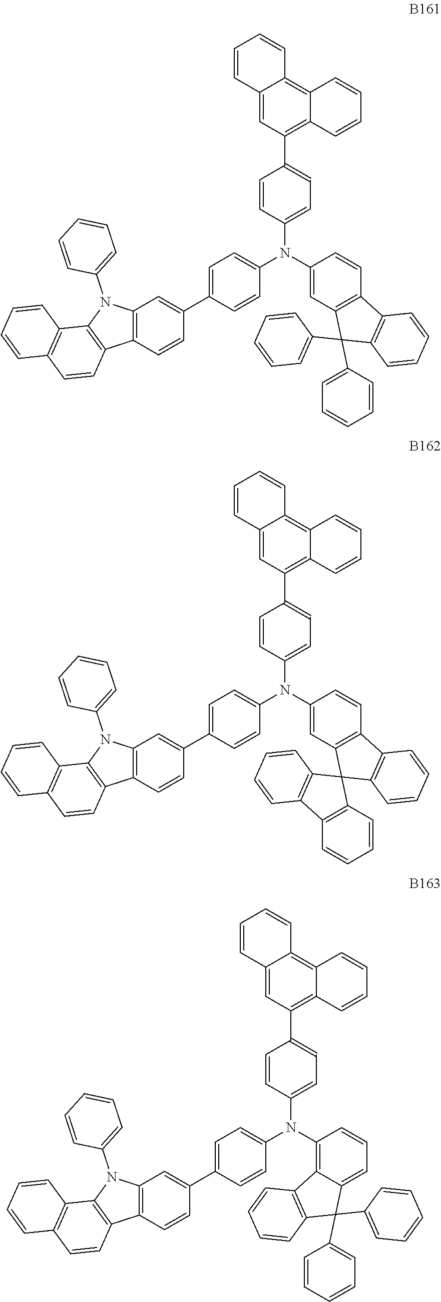 [SMILES: c1ccc(-n2c3cc(-c4ccc(N(c5ccc(-c6cc7ccccc7c7ccccc67)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6cc7ccccc7c7ccccc67)cc5)c5ccc6c(c5)C5(c7ccccc7-c7ccccc75)c5ccccc5-6)cc4)ccc3c3ccc4ccccc4c32)cc1 ; c1ccc(-n2c3cc(-c4ccc(N(c5ccc(-c6cc7ccccc7c7ccccc67)cc5)c5cccc6c5-c5ccccc5C6(c5ccccc5)c5ccccc5)cc4)ccc3c3ccc4ccccc4c32)cc1]

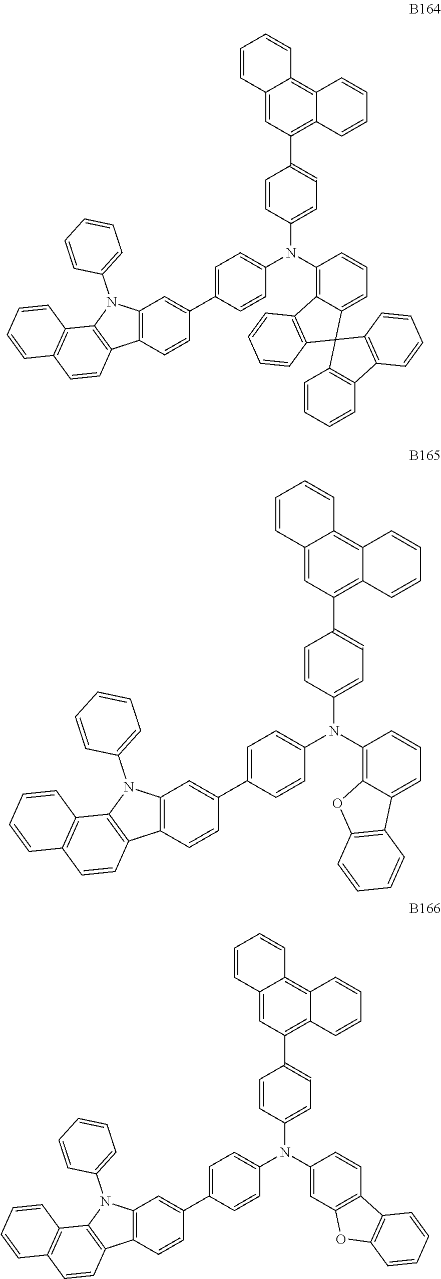 [SMILES: c1ccc(-n2c3cc(-c4ccc(N(c5ccc(-c6cc7ccccc7c7ccccc67)cc5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc(-c6cc7ccccc7c7ccccc67)cc5)c5cccc6c5oc5ccccc56)cc4)ccc3c3ccc4ccccc4c32)cc1 ; c1ccc(-n2c3cc(-c4ccc(N(c5ccc(-c6cc7ccccc7c7ccccc67)cc5)c5ccc6c(c5)oc5ccccc56)cc4)ccc3c3ccc4ccccc4c32)cc1]

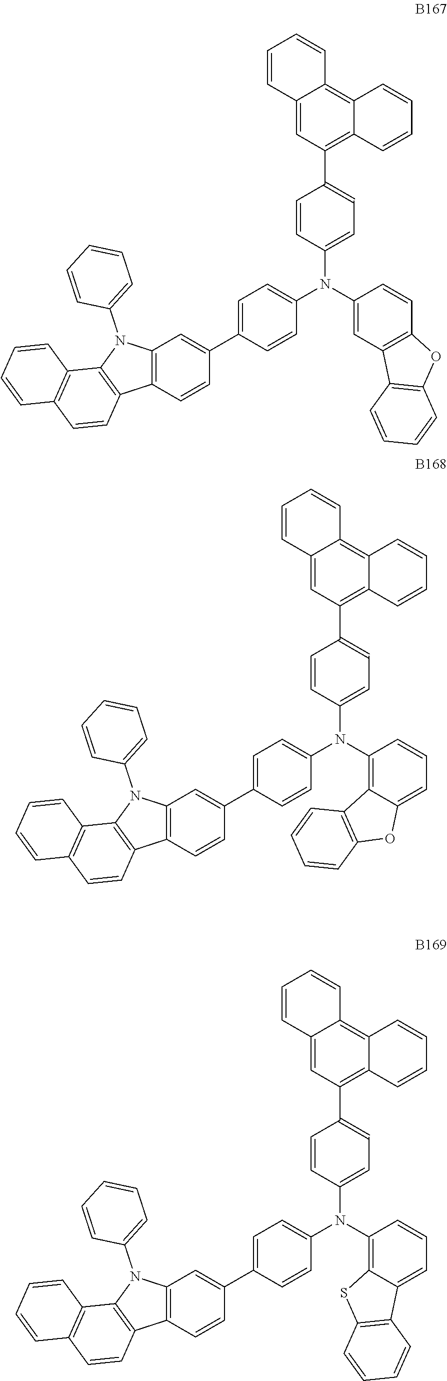 [SMILES: c1ccc(-n2c3cc(-c4ccc(N(c5ccc(-c6cc7ccccc7c7ccccc67)cc5)c5ccc6oc7ccccc7c6c5)cc4)ccc3c3ccc4ccccc4c32)cc1 ; c1ccc(-n2c3cc(-c4ccc(N(c5ccc(-c6cc7ccccc7c7ccccc67)cc5)c5cccc6oc7ccccc7c56)cc4)ccc3c3ccc4ccccc4c32)cc1 ; c1ccc(-n2c3cc(-c4ccc(N(c5ccc(-c6cc7ccccc7c7ccccc67)cc5)c5cccc6c5sc5ccccc56)cc4)ccc3c3ccc4ccccc4c32)cc1]

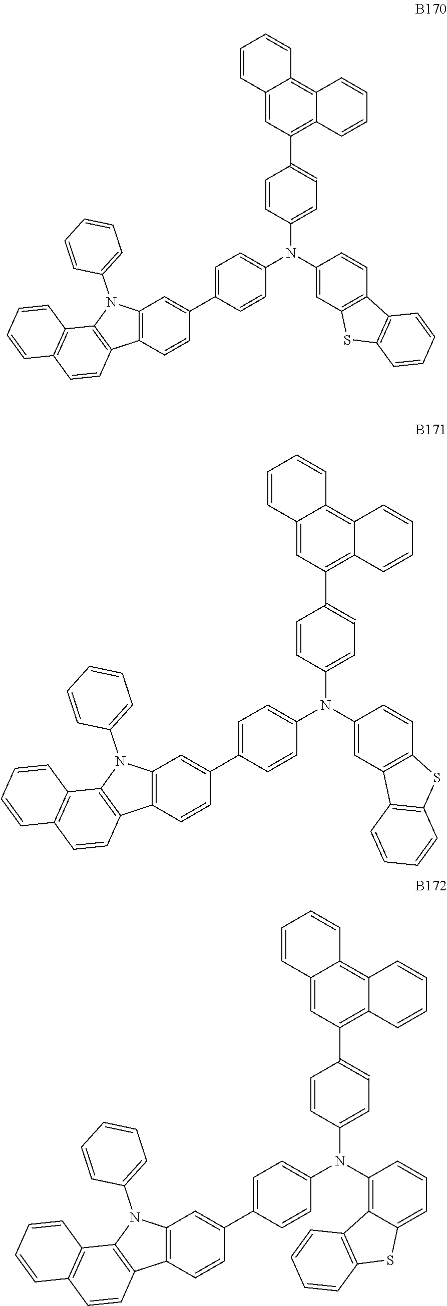 [SMILES: c1ccc(-n2c3cc(-c4ccc(N(c5ccc(-c6cc7ccccc7c7ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6cc7ccccc7c7ccccc67)cc5)c5ccc6sc7ccccc7c6c5)cc4)ccc3c3ccc4ccccc4c32)cc1 ; c1ccc(-n2c3cc(-c4ccc(N(c5ccc(-c6cc7ccccc7c7ccccc67)cc5)c5cccc6sc7ccccc7c56)cc4)ccc3c3ccc4ccccc4c32)cc1]

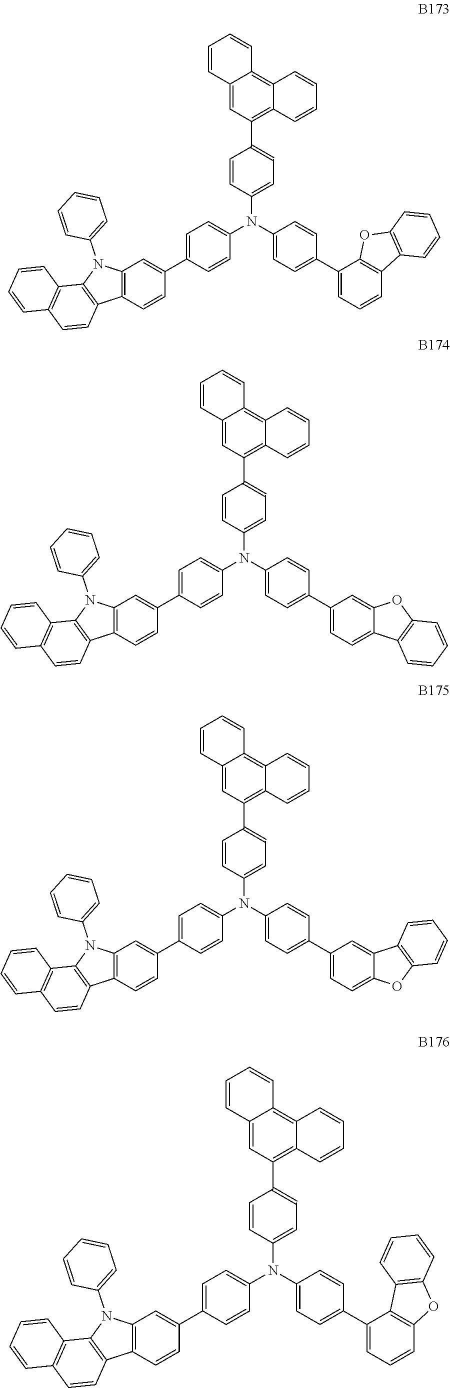 [SMILES: c1ccc(-n2c3cc(-c4ccc(N(c5ccc(-c6cc7ccccc7c7ccccc67)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7c(c6)oc6ccccc67)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6ccc7oc8ccccc8c7c6)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6cc7ccccc7c7ccccc67)cc5)c5ccc(-c6cccc7oc8ccccc8c67)cc5)cc4)ccc3c3ccc4ccccc4c32)cc1]

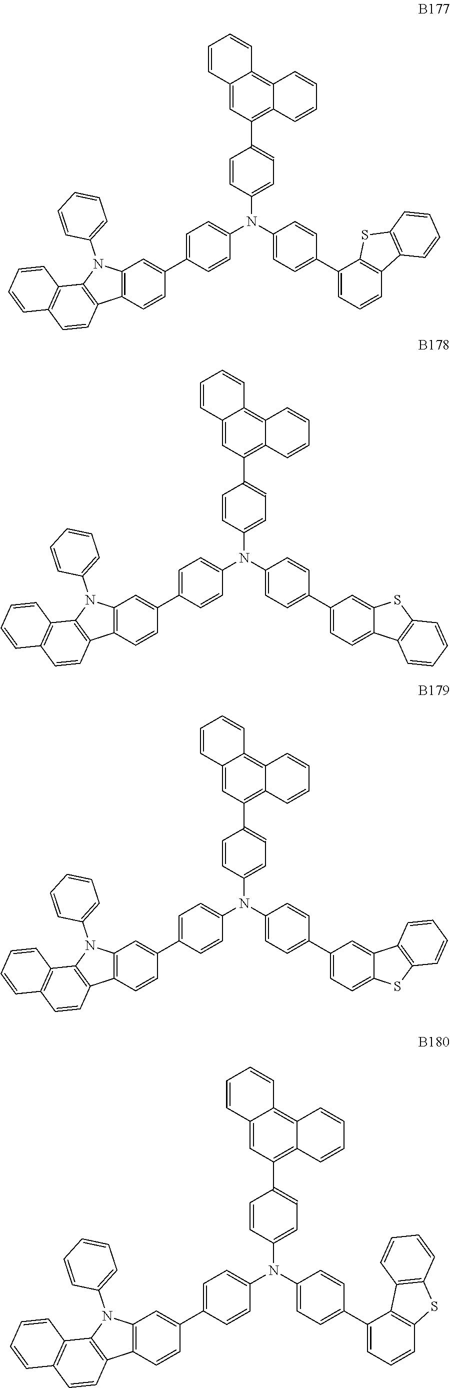 [SMILES: c1ccc(-n2c3cc(-c4ccc(N(c5ccc(-c6cc7ccccc7c7ccccc67)cc5)c5ccc(-c6cccc7c6sc6ccccc67)cc5)cc4)ccc3c3ccc4ccccc4c32)cc1 ; c1ccc(-n2c3cc(-c4ccc(N(c5ccc(-c6ccc7c(c6)sc6ccccc67)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6ccc7sc8ccccc8c7c6)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6cc7ccccc7c7ccccc67)cc5)c5ccc(-c6cccc7sc8ccccc8c67)cc5)cc4)ccc3c3ccc4ccccc4c32)cc1]

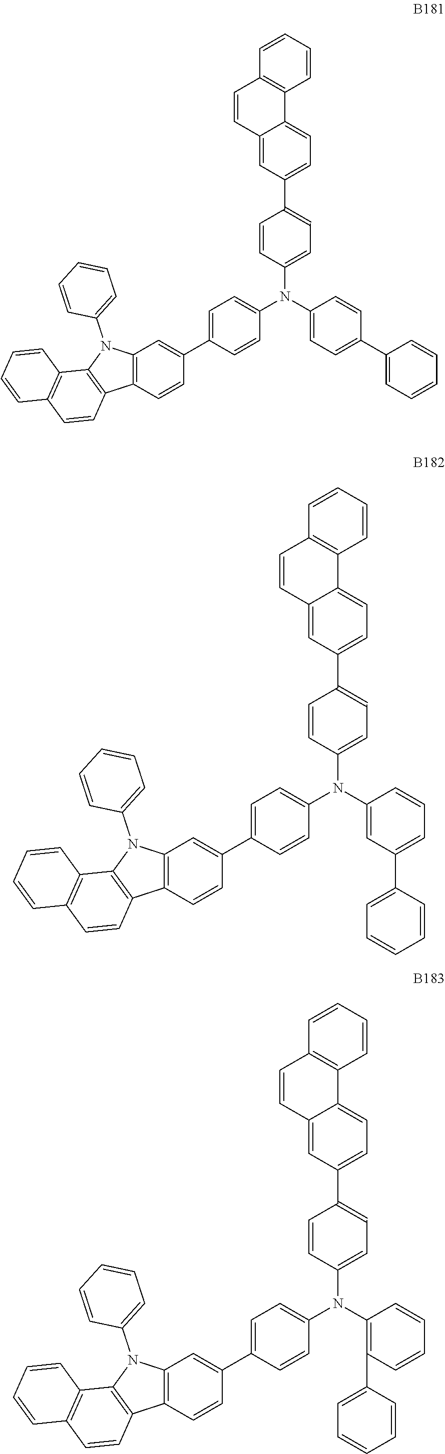 [SMILES: c1ccc(-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5c(ccc6ccccc65)c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4c(ccc5ccccc54)c3)cc2)c2ccc(-c3ccc4c5ccc6ccccc6c5n(-c5ccccc5)c4c3)cc2)cc1]

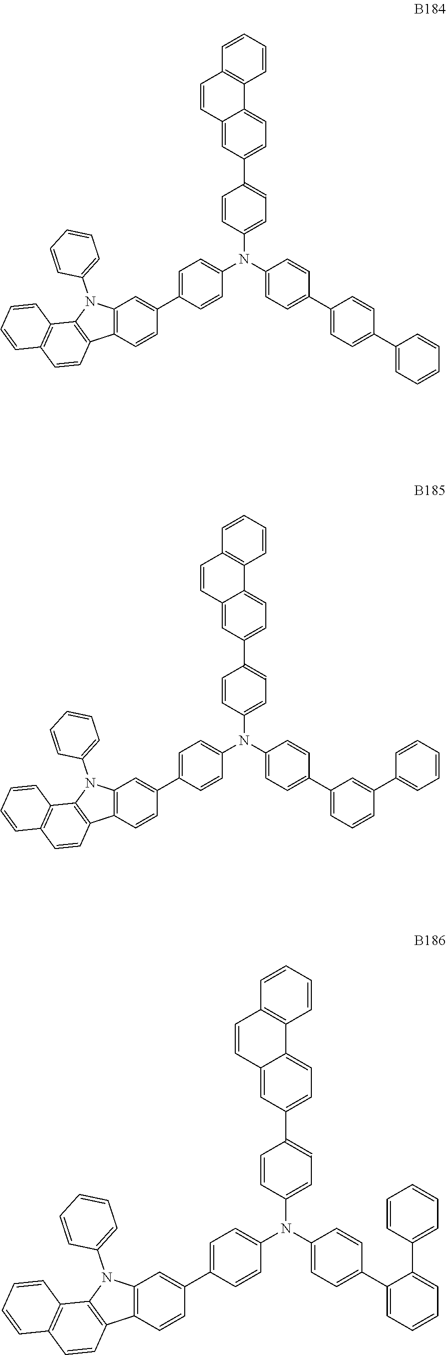 [SMILES: c1ccc(-c2ccc(-c3ccc(N(c4ccc(-c5ccc6c(ccc7ccccc76)c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6c(ccc7ccccc76)c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2)cc1]

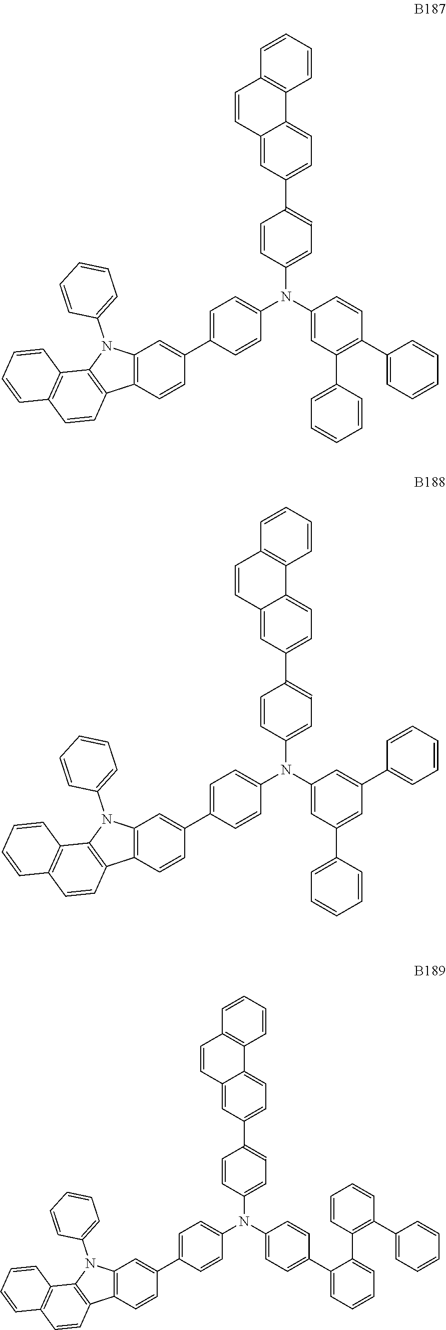 [SMILES: c1ccc(-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5c(ccc6ccccc65)c4)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2)cc1]

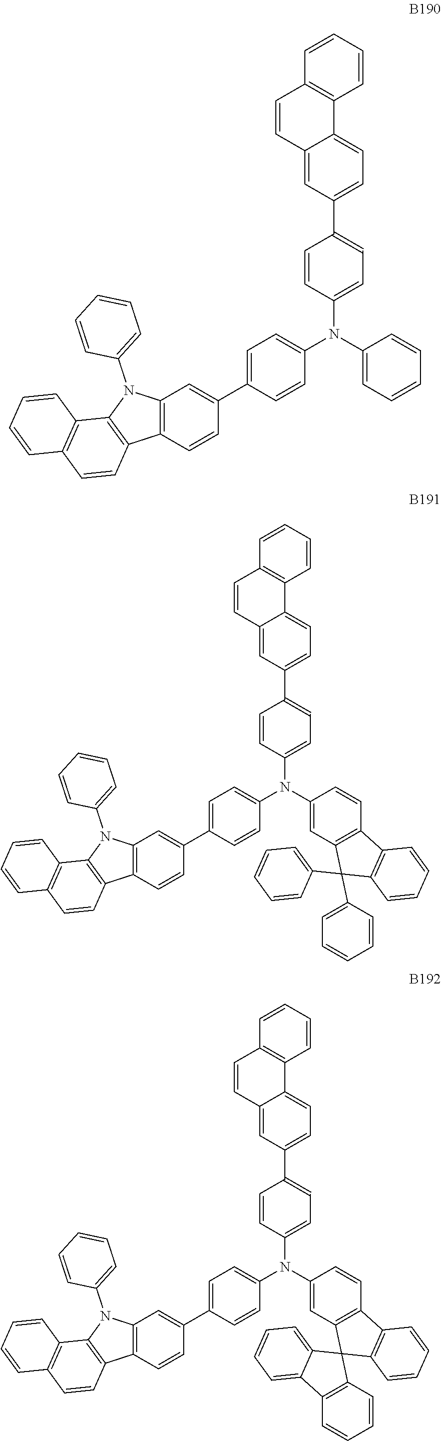 [SMILES: c1ccc(N(c2ccc(-c3ccc4c(ccc5ccccc54)c3)cc2)c2ccc(-c3ccc4c5ccc6ccccc6c5n(-c5ccccc5)c4c3)cc2)cc1 ; c1ccc(-n2c3cc(-c4ccc(N(c5ccc(-c6ccc7c(ccc8ccccc87)c6)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6c(c5)C5(c7ccccc7-c7ccccc75)c5ccccc5-6)cc4)ccc3c3ccc4ccccc4c32)cc1]

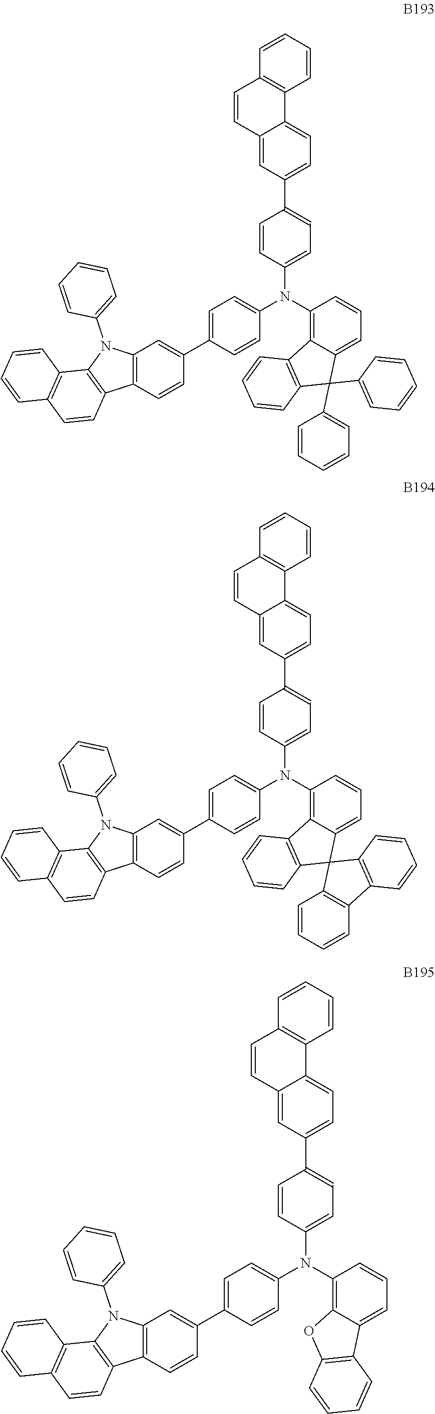

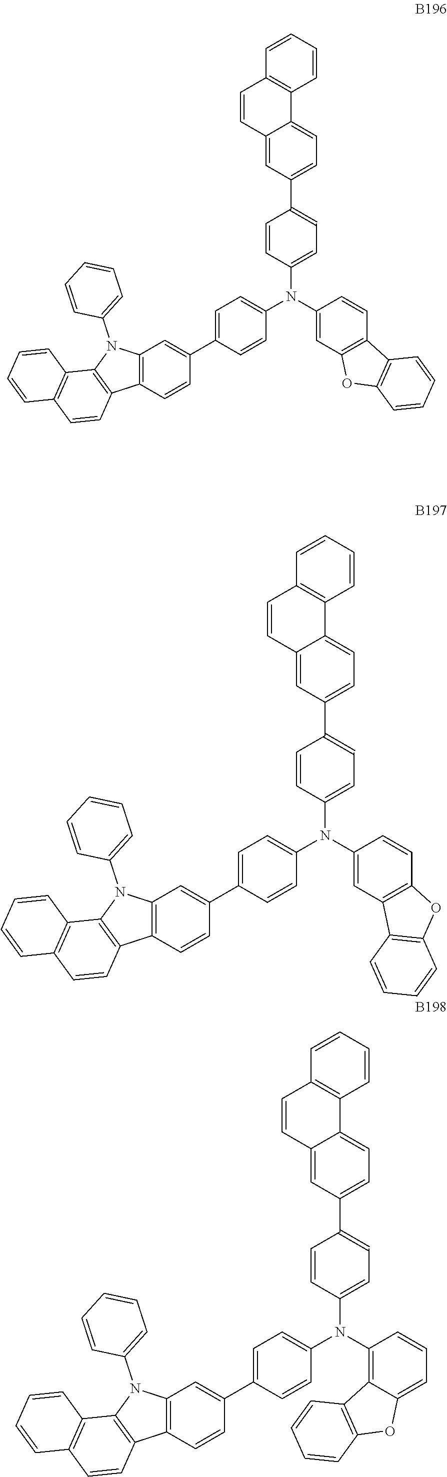 [SMILES: c1ccc(-n2c3cc(-c4ccc(N(c5ccc(-c6ccc7c(ccc8ccccc87)c6)cc5)c5ccc6c(c5)oc5ccccc56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6oc7ccccc7c6c5)cc4)ccc3c3ccc4ccccc4c32)cc1 ; c1ccc(-n2c3cc(-c4ccc(N(c5ccc(-c6ccc7c(ccc8ccccc87)c6)cc5)c5cccc6oc7ccccc7c56)cc4)ccc3c3ccc4ccccc4c32)cc1]

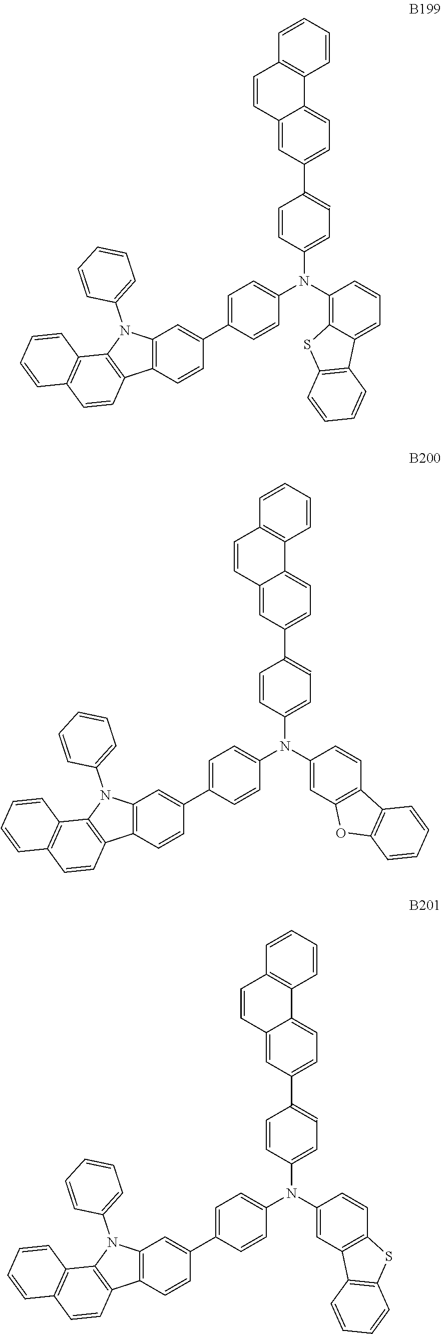 [SMILES: c1ccc(-n2c3cc(-c4ccc(N(c5ccc(-c6ccc7c(ccc8ccccc87)c6)cc5)c5cccc6c5sc5ccccc56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6c(c5)oc5ccccc56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6sc7ccccc7c6c5)cc4)ccc3c3ccc4ccccc4c32)cc1]

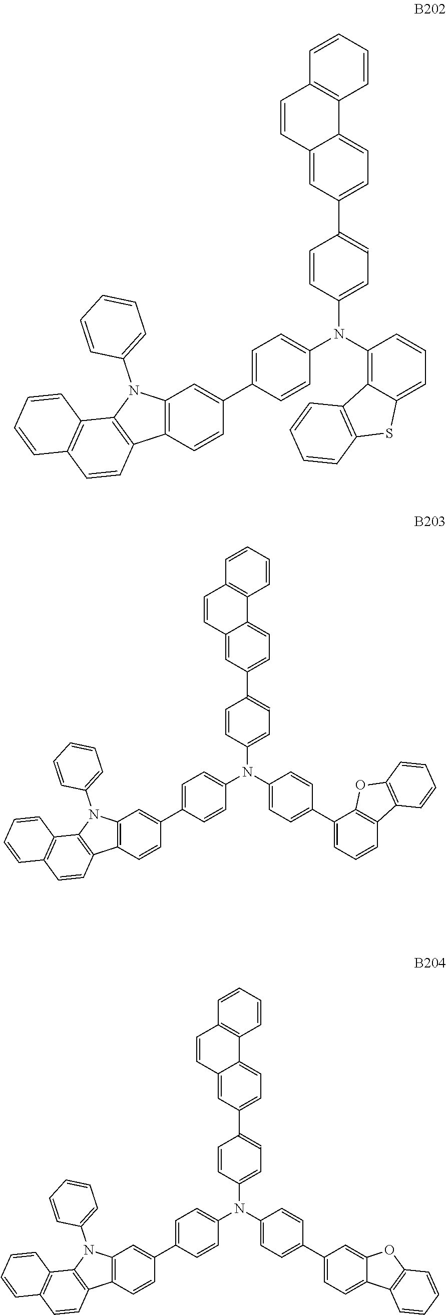 [SMILES: c1ccc(-n2c3cc(-c4ccc(N(c5ccc(-c6ccc7c(ccc8ccccc87)c6)cc5)c5cccc6sc7ccccc7c56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6ccc7c(c6)oc6ccccc67)cc5)cc4)ccc3c3ccc4ccccc4c32)cc1]

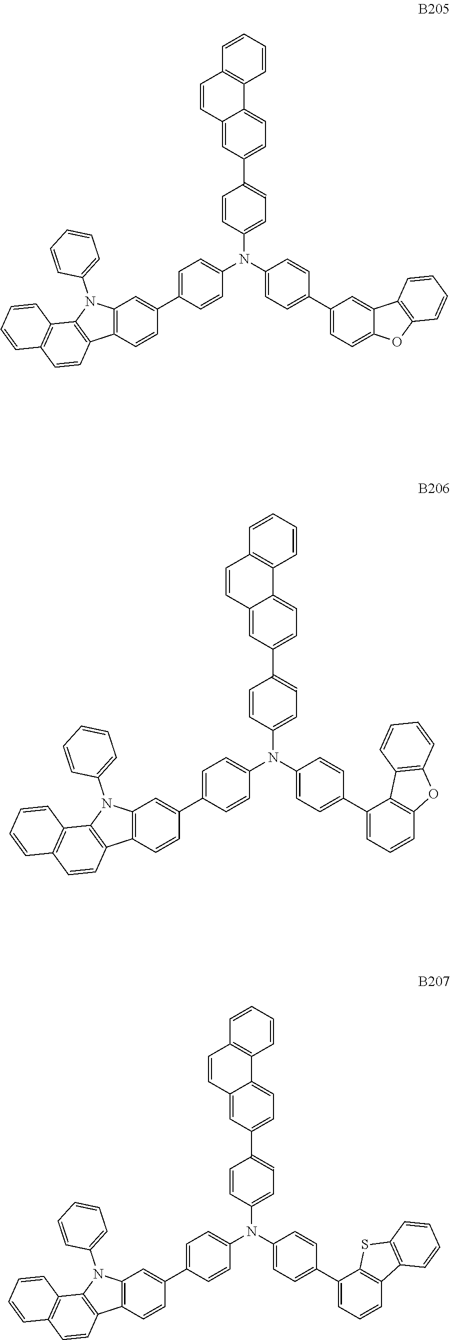 [SMILES: c1ccc(-n2c3cc(-c4ccc(N(c5ccc(-c6ccc7c(ccc8ccccc87)c6)cc5)c5ccc(-c6ccc7oc8ccccc8c7c6)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7oc8ccccc8c67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7c6sc6ccccc67)cc5)cc4)ccc3c3ccc4ccccc4c32)cc1]

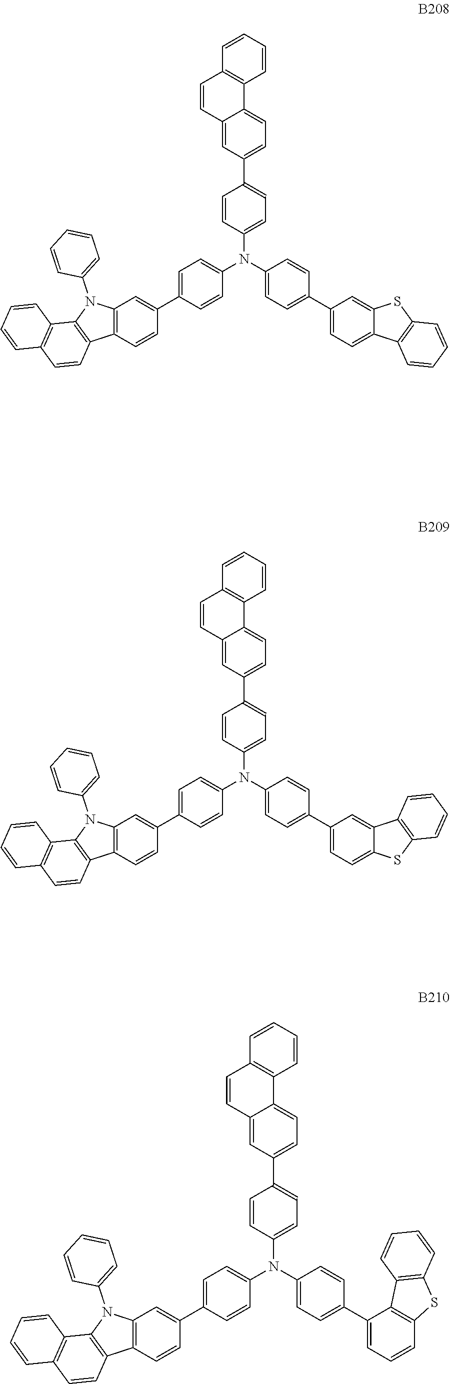 [SMILES: c1ccc(-n2c3cc(-c4ccc(N(c5ccc(-c6ccc7c(ccc8ccccc87)c6)cc5)c5ccc(-c6ccc7c(c6)sc6ccccc67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6ccc7sc8ccccc8c7c6)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7sc8ccccc8c67)cc5)cc4)ccc3c3ccc4ccccc4c32)cc1]

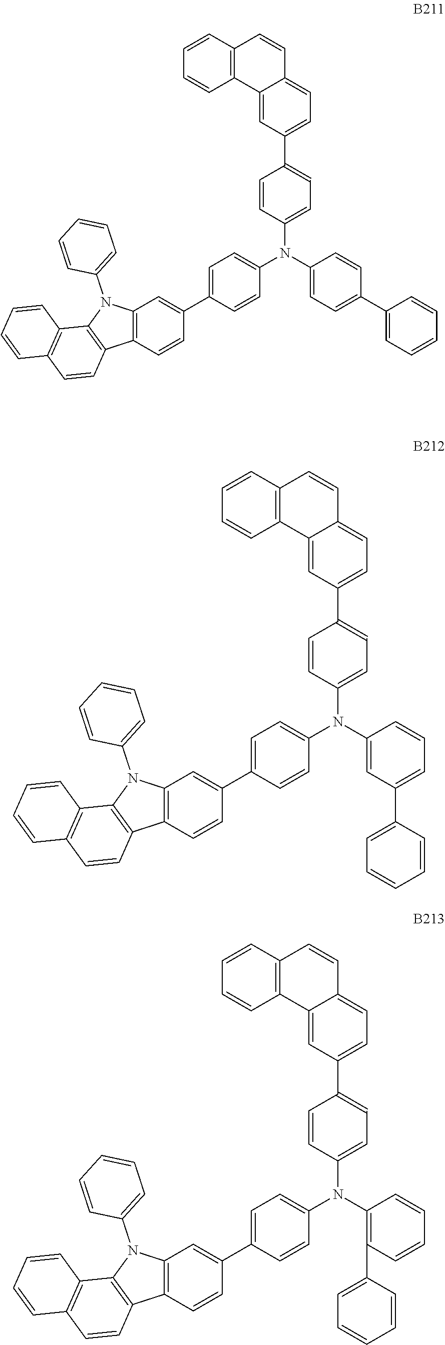 [SMILES: c1ccc(-c2ccc(N(c3ccc(-c4ccc5ccc6ccccc6c5c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5ccc6ccccc6c5c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4ccc5ccccc5c4c3)cc2)c2ccc(-c3ccc4c5ccc6ccccc6c5n(-c5ccccc5)c4c3)cc2)cc1]

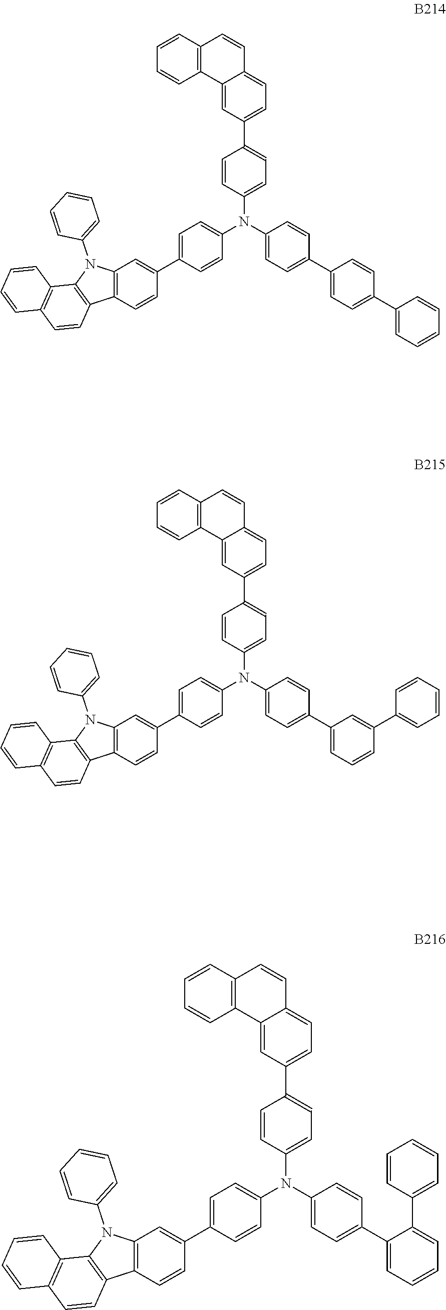 [SMILES: c1ccc(-c2ccc(-c3ccc(N(c4ccc(-c5ccc6ccc7ccccc7c6c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6ccc7ccccc7c6c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5ccc6ccccc6c5c4)cc3)c3ccc(-c4ccc5c6ccc7ccccc7c6n(-c6ccccc6)c5c4)cc3)cc2)cc1]

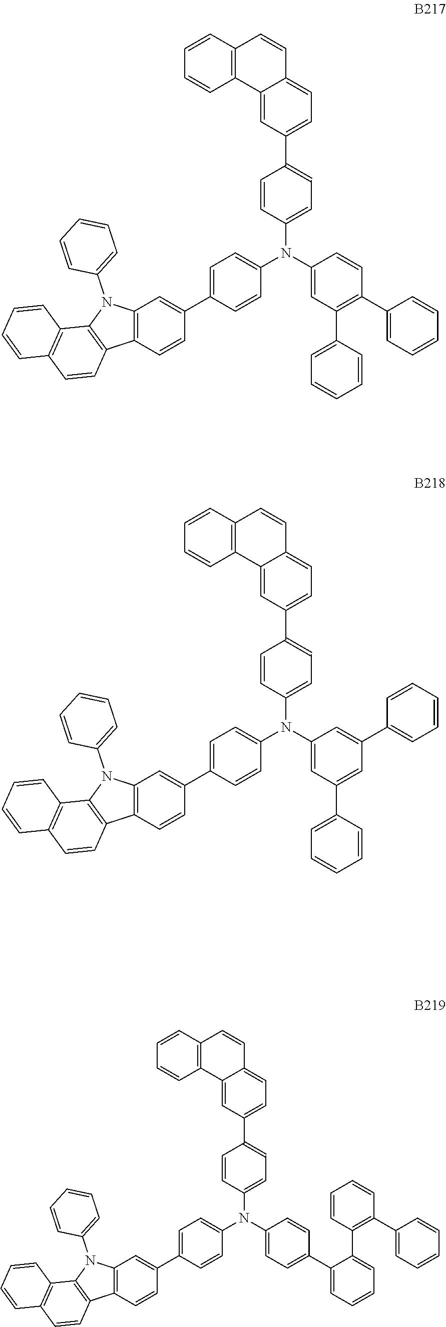 [SMILES: c1ccc(-c2ccc(N(c3ccc(-c4ccc5ccc6ccccc6c5c4)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5ccc6ccccc6c5c4)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5ccc6ccccc6c5c4)cc3)c3ccc(-c4ccc5c6ccc7ccccc7c6n(-c6ccccc6)c5c4)cc3)cc2)cc1]

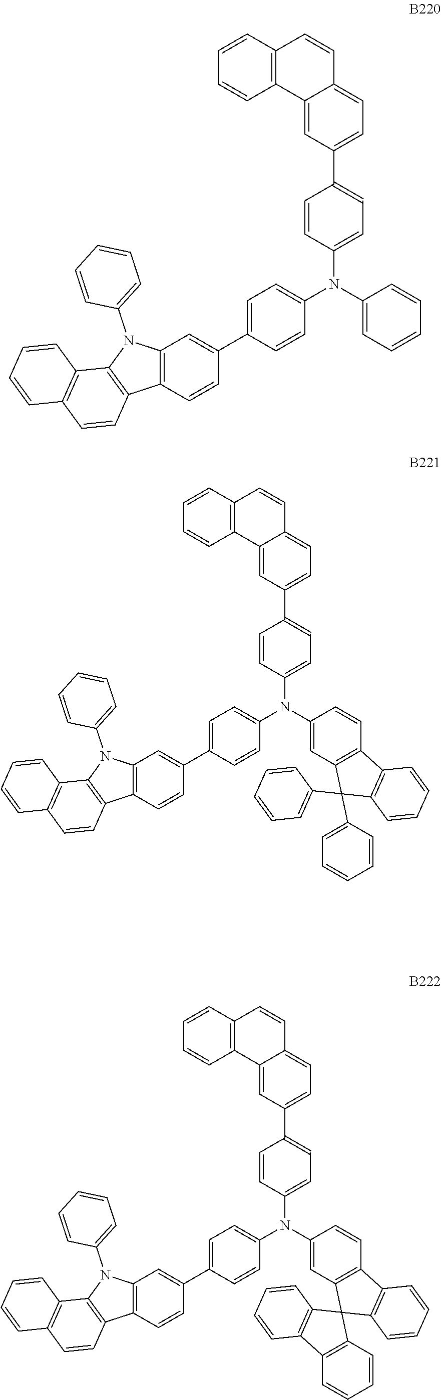 [SMILES: c1ccc(N(c2ccc(-c3ccc4ccc5ccccc5c4c3)cc2)c2ccc(-c3ccc4c5ccc6ccccc6c5n(-c5ccccc5)c4c3)cc2)cc1 ; c1ccc(-n2c3cc(-c4ccc(N(c5ccc(-c6ccc7ccc8ccccc8c7c6)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6ccc7ccc8ccccc8c7c6)cc5)c5ccc6c(c5)C5(c7ccccc7-c7ccccc75)c5ccccc5-6)cc4)ccc3c3ccc4ccccc4c32)cc1]

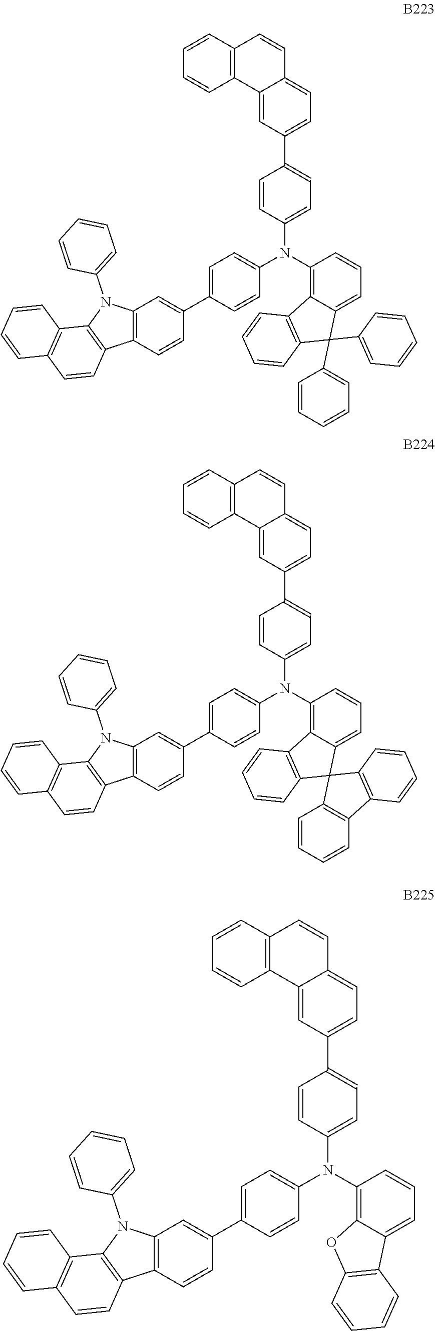 [SMILES: c1ccc(-n2c3cc(-c4ccc(N(c5ccc(-c6ccc7ccc8ccccc8c7c6)cc5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc(-c6ccc7ccc8ccccc8c7c6)cc5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc(-c6ccc7ccc8ccccc8c7c6)cc5)c5cccc6c5oc5ccccc56)cc4)ccc3c3ccc4ccccc4c32)cc1]

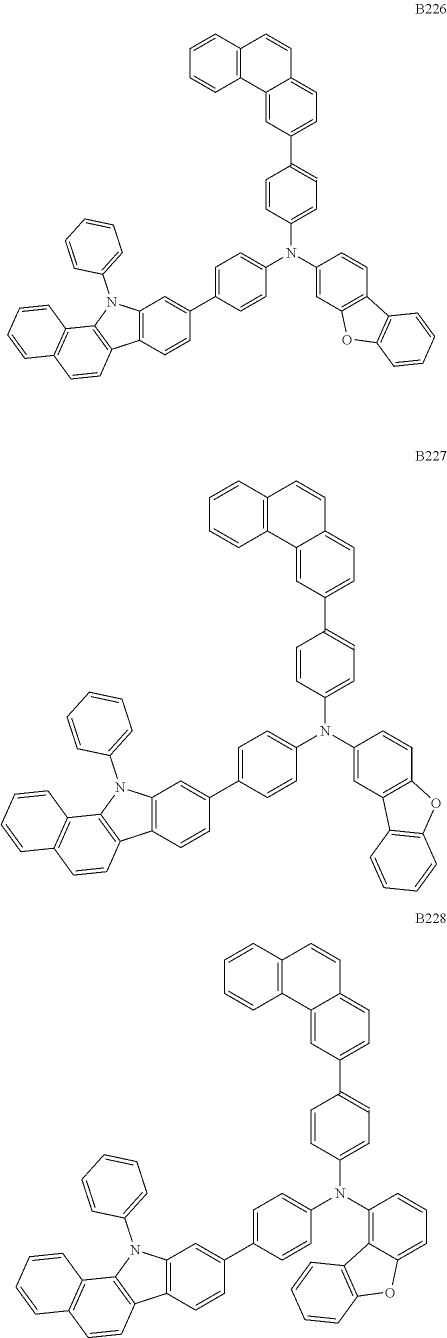 [SMILES: c1ccc(-n2c3cc(-c4ccc(N(c5ccc(-c6ccc7ccc8ccccc8c7c6)cc5)c5ccc6c(c5)oc5ccccc56)cc4)ccc3c3ccc4ccccc4c32)cc1 ; c1ccc(-n2c3cc(-c4ccc(N(c5ccc(-c6ccc7ccc8ccccc8c7c6)cc5)c5ccc6oc7ccccc7c6c5)cc4)ccc3c3ccc4ccccc4c32)cc1 ; c1ccc(-n2c3cc(-c4ccc(N(c5ccc(-c6ccc7ccc8ccccc8c7c6)cc5)c5cccc6oc7ccccc7c56)cc4)ccc3c3ccc4ccccc4c32)cc1]

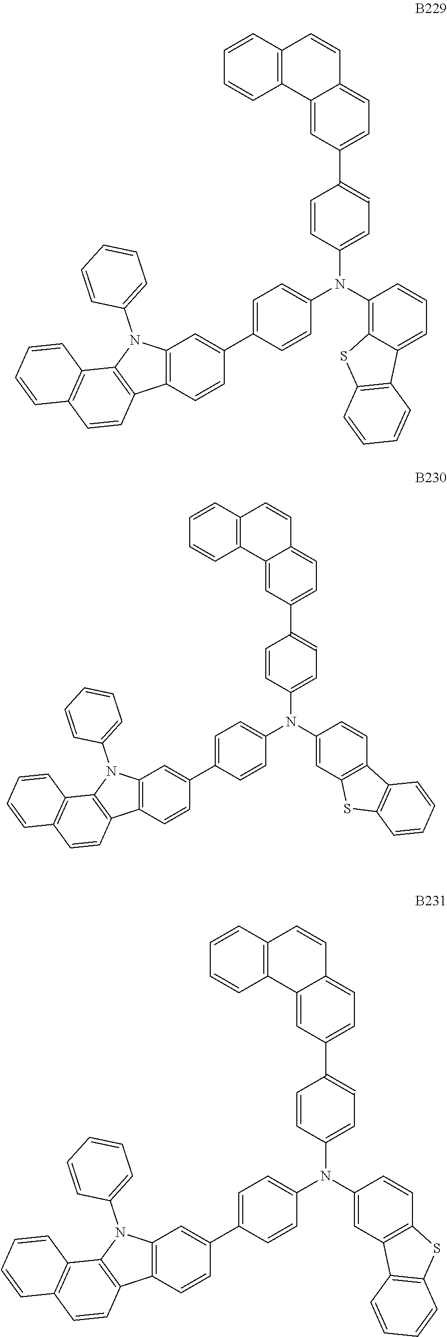 [SMILES: c1ccc(-n2c3cc(-c4ccc(N(c5ccc(-c6ccc7ccc8ccccc8c7c6)cc5)c5cccc6c5sc5ccccc56)cc4)ccc3c3ccc4ccccc4c32)cc1 ; c1ccc(-n2c3cc(-c4ccc(N(c5ccc(-c6ccc7ccc8ccccc8c7c6)cc5)c5ccc6c(c5)sc5ccccc56)cc4)ccc3c3ccc4ccccc4c32)cc1 ; c1ccc(-n2c3cc(-c4ccc(N(c5ccc(-c6ccc7ccc8ccccc8c7c6)cc5)c5ccc6sc7ccccc7c6c5)cc4)ccc3c3ccc4ccccc4c32)cc1]

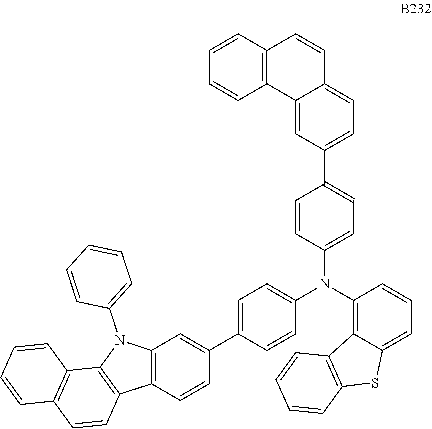 [SMILES: c1ccc(-n2c3cc(-c4ccc(N(c5ccc(-c6ccc7ccc8ccccc8c7c6)cc5)c5cccc6sc7ccccc7c56)cc4)ccc3c3ccc4ccccc4c32)cc1]

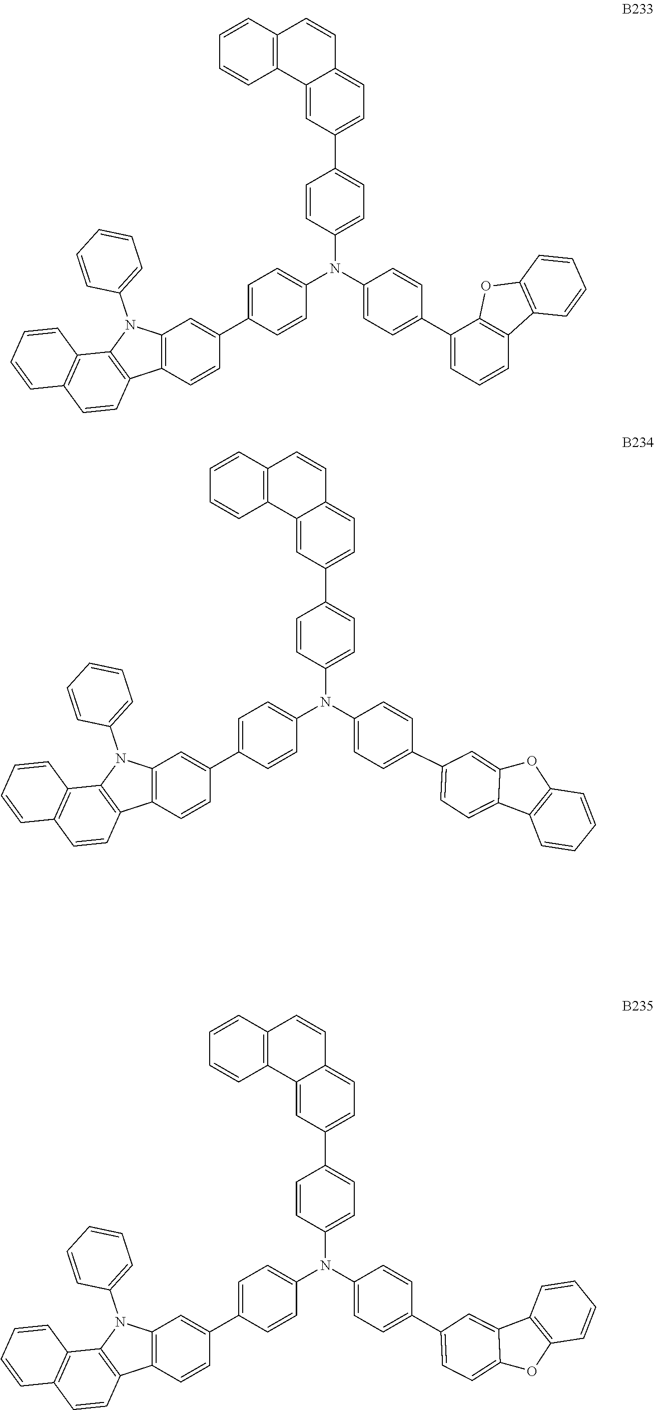 [SMILES: c1ccc(-n2c3cc(-c4ccc(N(c5ccc(-c6ccc7ccc8ccccc8c7c6)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7c(c6)oc6ccccc67)cc5)c5ccc(-c6ccc7ccc8ccccc8c7c6)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6ccc7oc8ccccc8c7c6)cc5)cc4)ccc3c3ccc4ccccc4c32)cc1]

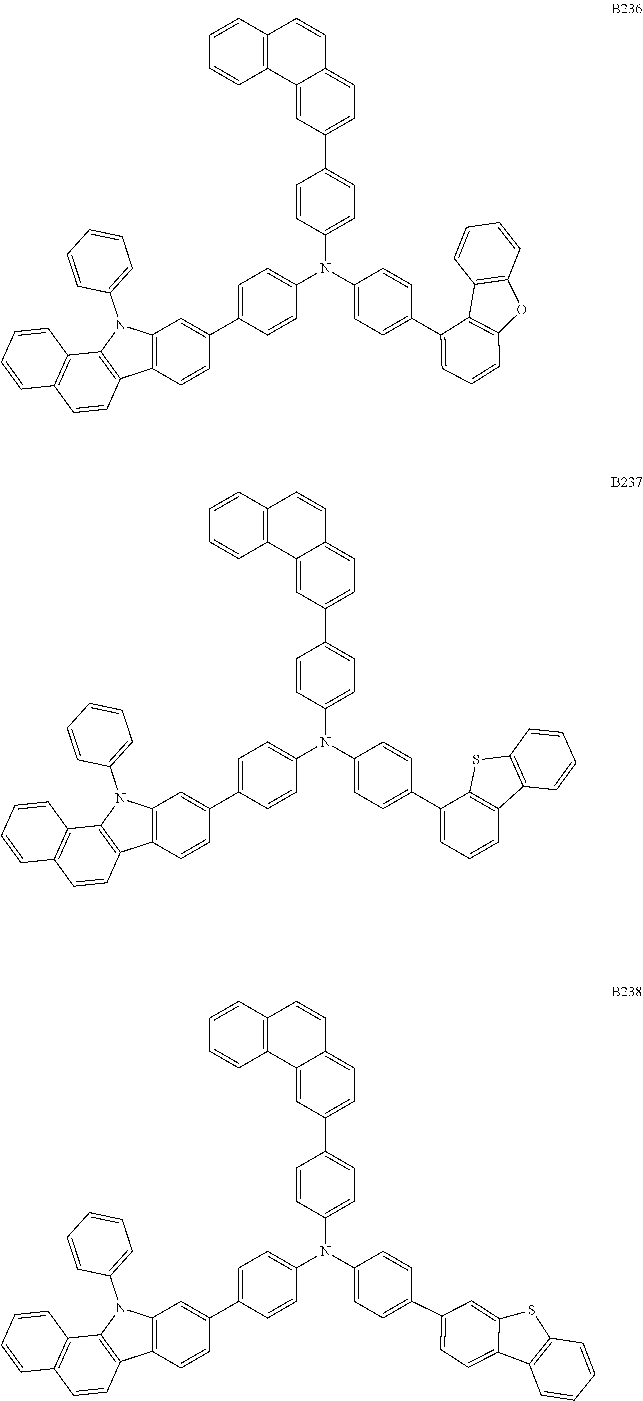 [SMILES: c1ccc(-n2c3cc(-c4ccc(N(c5ccc(-c6ccc7ccc8ccccc8c7c6)cc5)c5ccc(-c6cccc7oc8ccccc8c67)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6cccc7c6sc6ccccc67)cc5)cc4)ccc3c3ccc4ccccc4c32)cc1 ; c1ccc(-n2c3cc(-c4ccc(N(c5ccc(-c6ccc7c(c6)sc6ccccc67)cc5)c5ccc(-c6ccc7ccc8ccccc8c7c6)cc5)cc4)ccc3c3ccc4ccccc4c32)cc1]

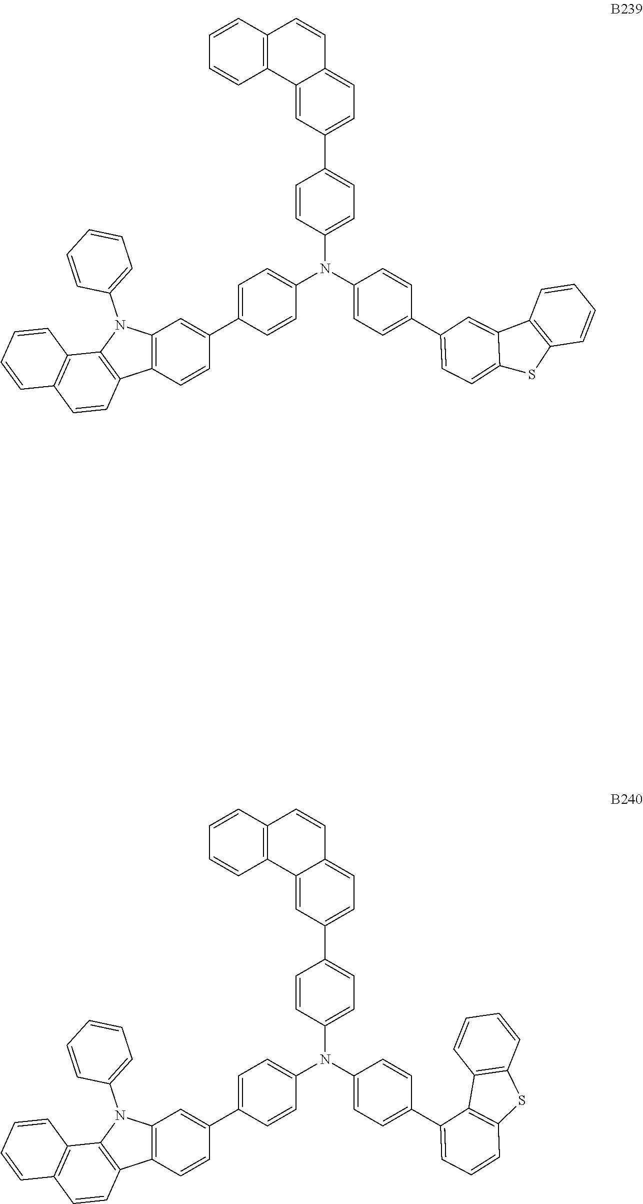 [SMILES: c1ccc(-n2c3cc(-c4ccc(N(c5ccc(-c6ccc7ccc8ccccc8c7c6)cc5)c5ccc(-c6ccc7sc8ccccc8c7c6)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6cccc7sc8ccccc8c67)cc5)cc4)ccc3c3ccc4ccccc4c32)cc1]

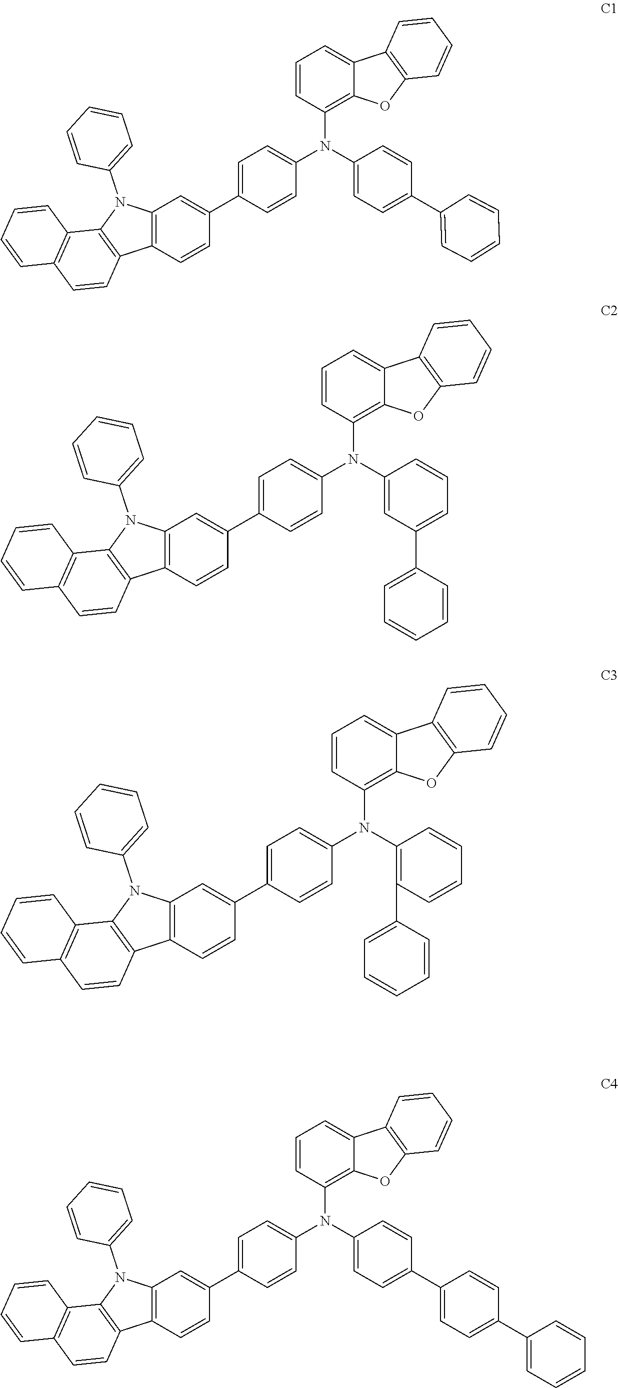 [SMILES: c1ccc(-c2ccc(N(c3ccc(-c4ccc5c6ccc7ccccc7c6n(-c6ccccc6)c5c4)cc3)c3cccc4c3oc3ccccc34)cc2)cc1 ; c1ccc(-c2cccc(N(c3ccc(-c4ccc5c6ccc7ccccc7c6n(-c6ccccc6)c5c4)cc3)c3cccc4c3oc3ccccc34)c2)cc1 ; c1ccc(-c2ccccc2N(c2ccc(-c3ccc4c5ccc6ccccc6c5n(-c5ccccc5)c4c3)cc2)c2cccc3c2oc2ccccc23)cc1 ; c1ccc(-c2ccc(-c3ccc(N(c4ccc(-c5ccc6c7ccc8ccccc8c7n(-c7ccccc7)c6c5)cc4)c4cccc5c4oc4ccccc45)cc3)cc2)cc1]

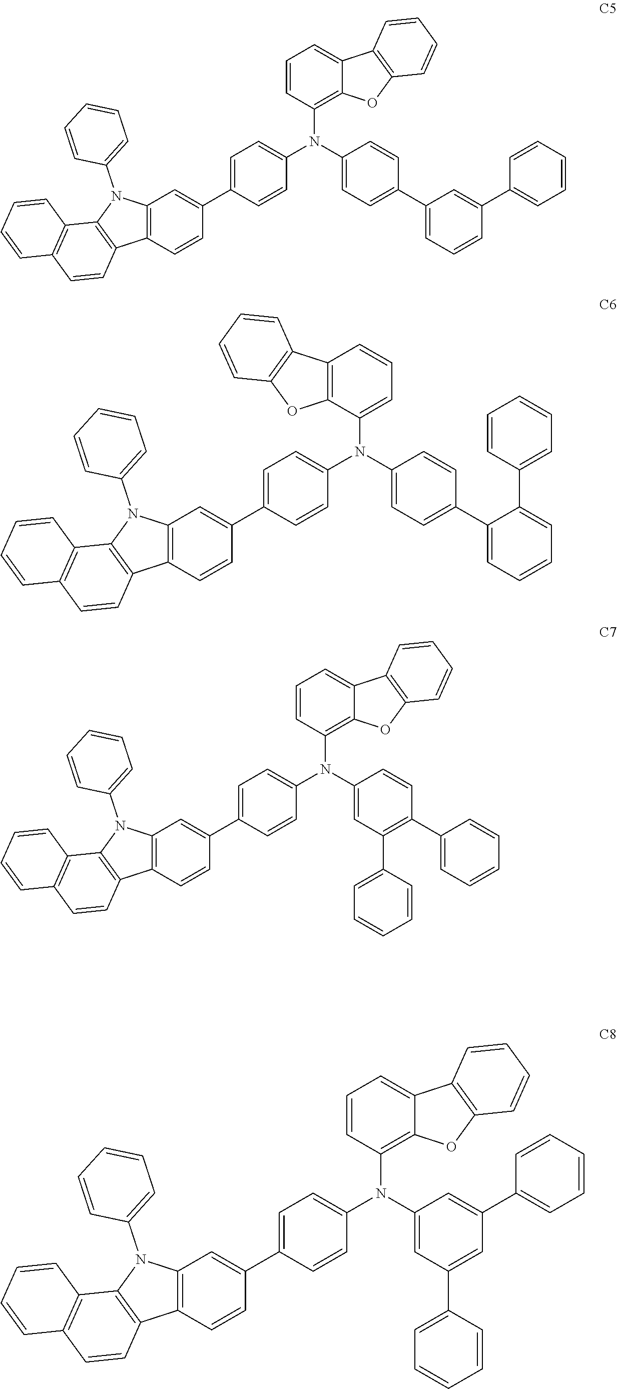 [SMILES: c1ccc(-c2cccc(-c3ccc(N(c4ccc(-c5ccc6c7ccc8ccccc8c7n(-c7ccccc7)c6c5)cc4)c4cccc5c4oc4ccccc45)cc3)c2)cc1 ; c1ccc(-c2ccccc2-c2ccc(N(c3ccc(-c4ccc5c6ccc7ccccc7c6n(-c6ccccc6)c5c4)cc3)c3cccc4c3oc3ccccc34)cc2)cc1 ; c1ccc(-c2ccc(N(c3ccc(-c4ccc5c6ccc7ccccc7c6n(-c6ccccc6)c5c4)cc3)c3cccc4c3oc3ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4c3oc3ccccc34)c2)cc1]

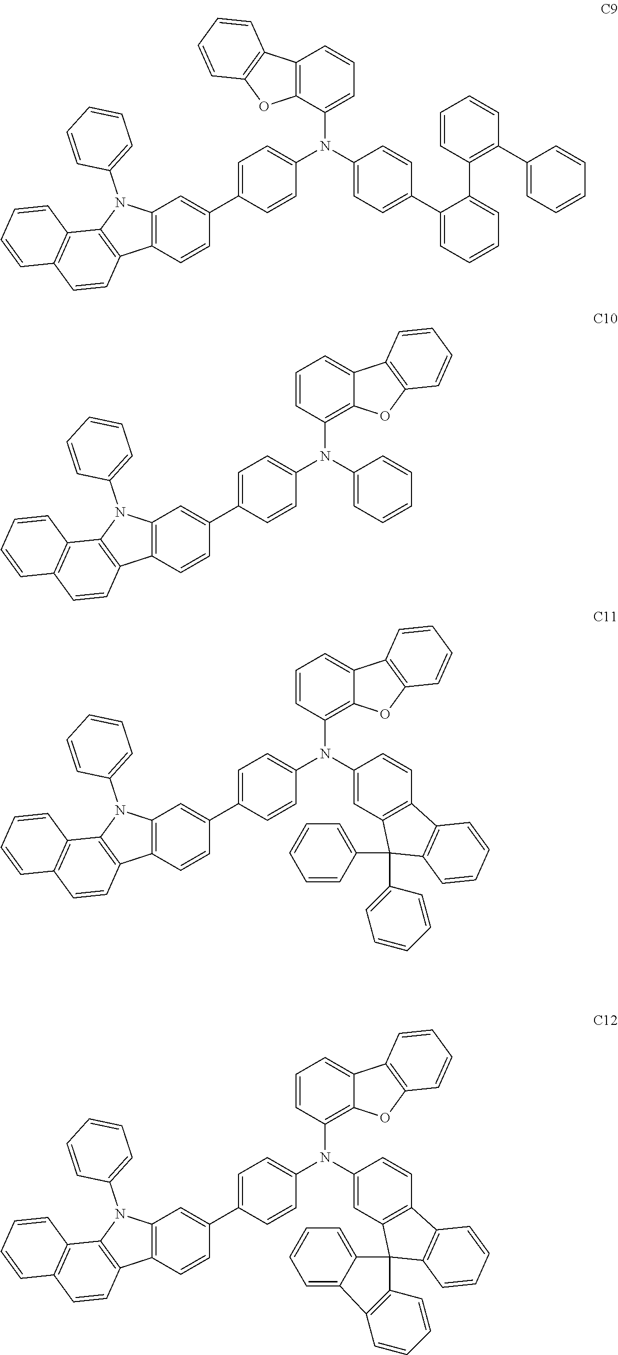 [SMILES: c1ccc(-c2ccccc2-c2ccccc2-c2ccc(N(c3ccc(-c4ccc5c6ccc7ccccc7c6n(-c6ccccc6)c5c4)cc3)c3cccc4c3oc3ccccc34)cc2)cc1 ; c1ccc(N(c2ccc(-c3ccc4c5ccc6ccccc6c5n(-c5ccccc5)c4c3)cc2)c2cccc3c2oc2ccccc23)cc1 ; c1ccc(-n2c3cc(-c4ccc(N(c5ccc6c(c5)C(c5ccccc5)(c5ccccc5)c5ccccc5-6)c5cccc6c5oc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5cccc6c5oc5ccccc56)cc4)ccc3c3ccc4ccccc4c32)cc1]

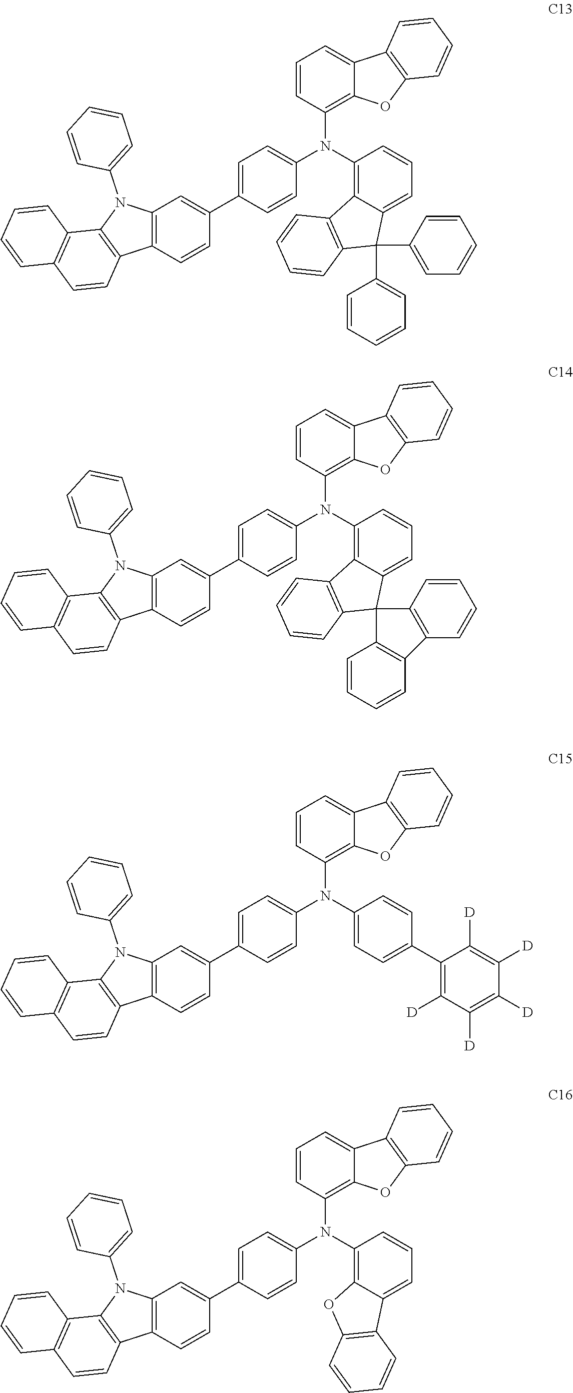

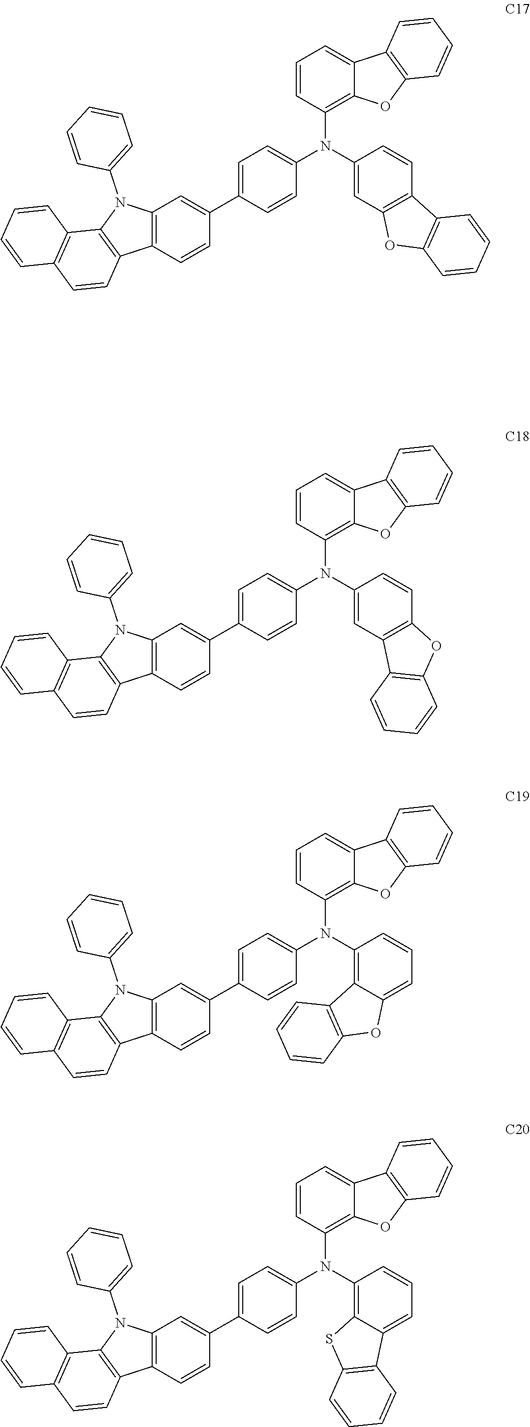 [SMILES: c1ccc(-n2c3cc(-c4ccc(N(c5ccc6c(c5)oc5ccccc56)c5cccc6c5oc5ccccc56)cc4)ccc3c3ccc4ccccc4c32)cc1 ; c1ccc(-n2c3cc(-c4ccc(N(c5ccc6oc7ccccc7c6c5)c5cccc6c5oc5ccccc56)cc4)ccc3c3ccc4ccccc4c32)cc1 ; c1ccc(-n2c3cc(-c4ccc(N(c5cccc6c5oc5ccccc56)c5cccc6oc7ccccc7c56)cc4)ccc3c3ccc4ccccc4c32)cc1 ; c1ccc(-n2c3cc(-c4ccc(N(c5cccc6c5oc5ccccc56)c5cccc6c5sc5ccccc56)cc4)ccc3c3ccc4ccccc4c32)cc1]

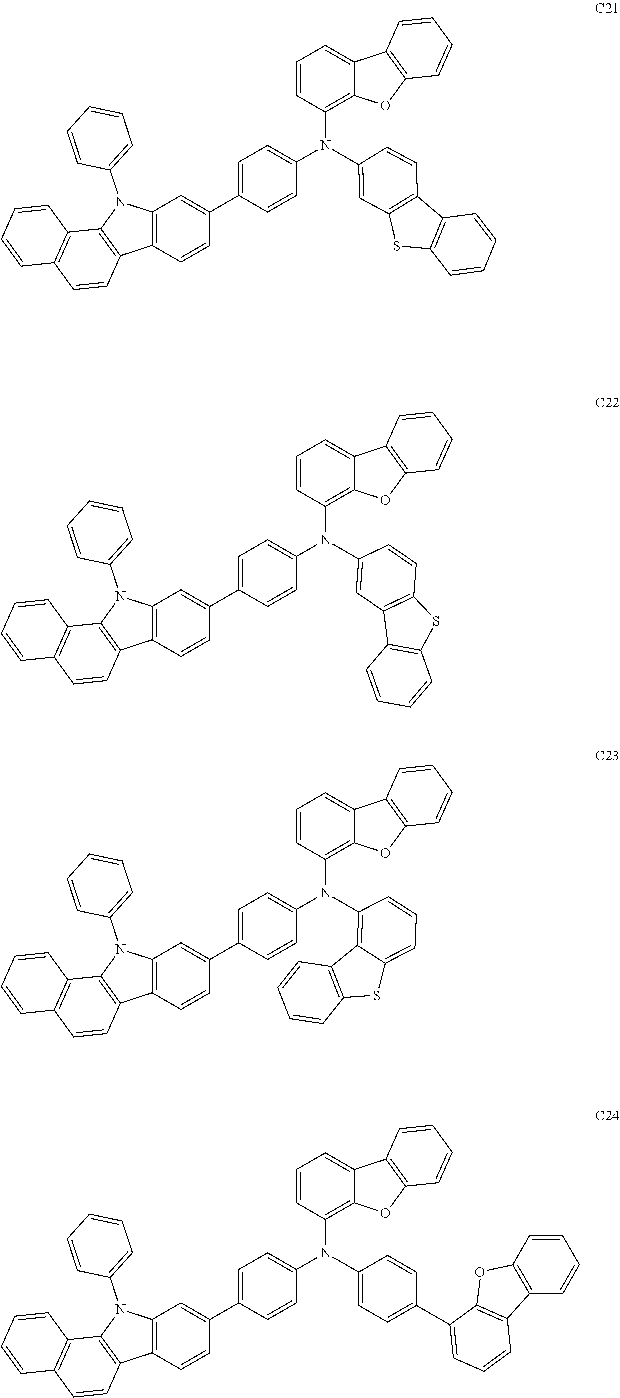 [SMILES: c1ccc(-n2c3cc(-c4ccc(N(c5ccc6c(c5)sc5ccccc56)c5cccc6c5oc5ccccc56)cc4)ccc3c3ccc4ccccc4c32)cc1 ; c1ccc(-n2c3cc(-c4ccc(N(c5ccc6sc7ccccc7c6c5)c5cccc6c5oc5ccccc56)cc4)ccc3c3ccc4ccccc4c32)cc1 ; c1ccc(-n2c3cc(-c4ccc(N(c5cccc6c5oc5ccccc56)c5cccc6sc7ccccc7c56)cc4)ccc3c3ccc4ccccc4c32)cc1 ; c1ccc(-n2c3cc(-c4ccc(N(c5ccc(-c6cccc7c6oc6ccccc67)cc5)c5cccc6c5oc5ccccc56)cc4)ccc3c3ccc4ccccc4c32)cc1]

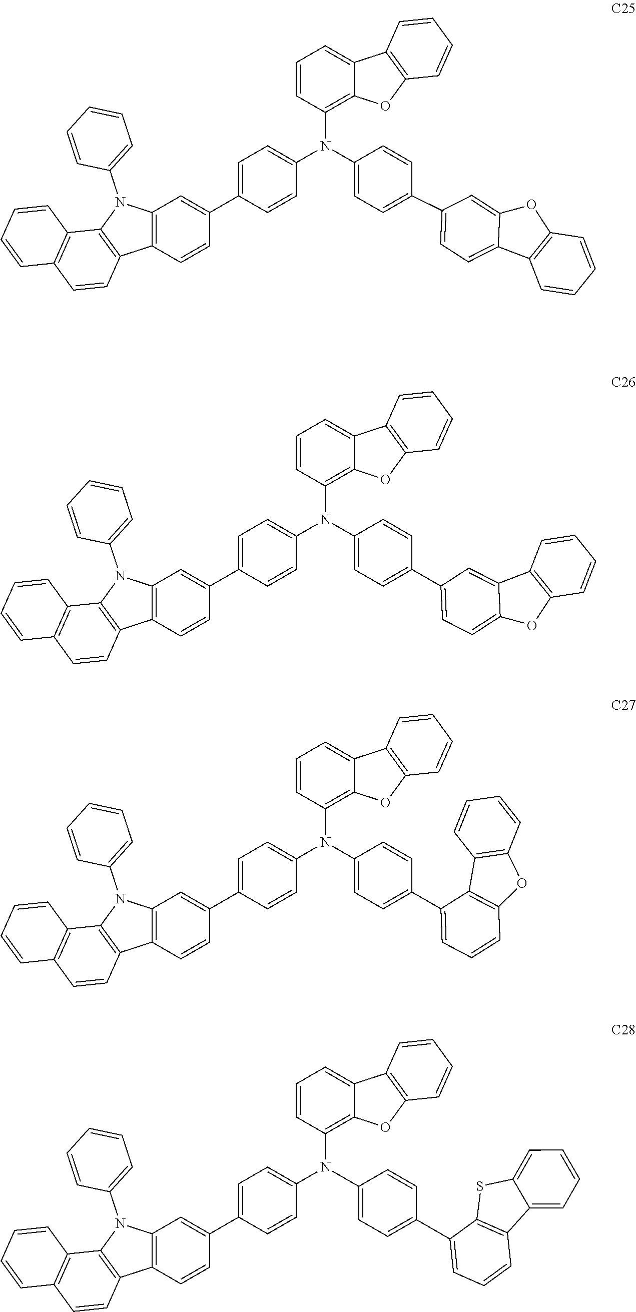 [SMILES: c1ccc(-n2c3cc(-c4ccc(N(c5ccc(-c6ccc7c(c6)oc6ccccc67)cc5)c5cccc6c5oc5ccccc56)cc4)ccc3c3ccc4ccccc4c32)cc1 ; c1ccc(-n2c3cc(-c4ccc(N(c5ccc(-c6ccc7oc8ccccc8c7c6)cc5)c5cccc6c5oc5ccccc56)cc4)ccc3c3ccc4ccccc4c32)cc1 ; c1ccc(-n2c3cc(-c4ccc(N(c5ccc(-c6cccc7oc8ccccc8c67)cc5)c5cccc6c5oc5ccccc56)cc4)ccc3c3ccc4ccccc4c32)cc1 ; c1ccc(-n2c3cc(-c4ccc(N(c5ccc(-c6cccc7c6sc6ccccc67)cc5)c5cccc6c5oc5ccccc56)cc4)ccc3c3ccc4ccccc4c32)cc1]

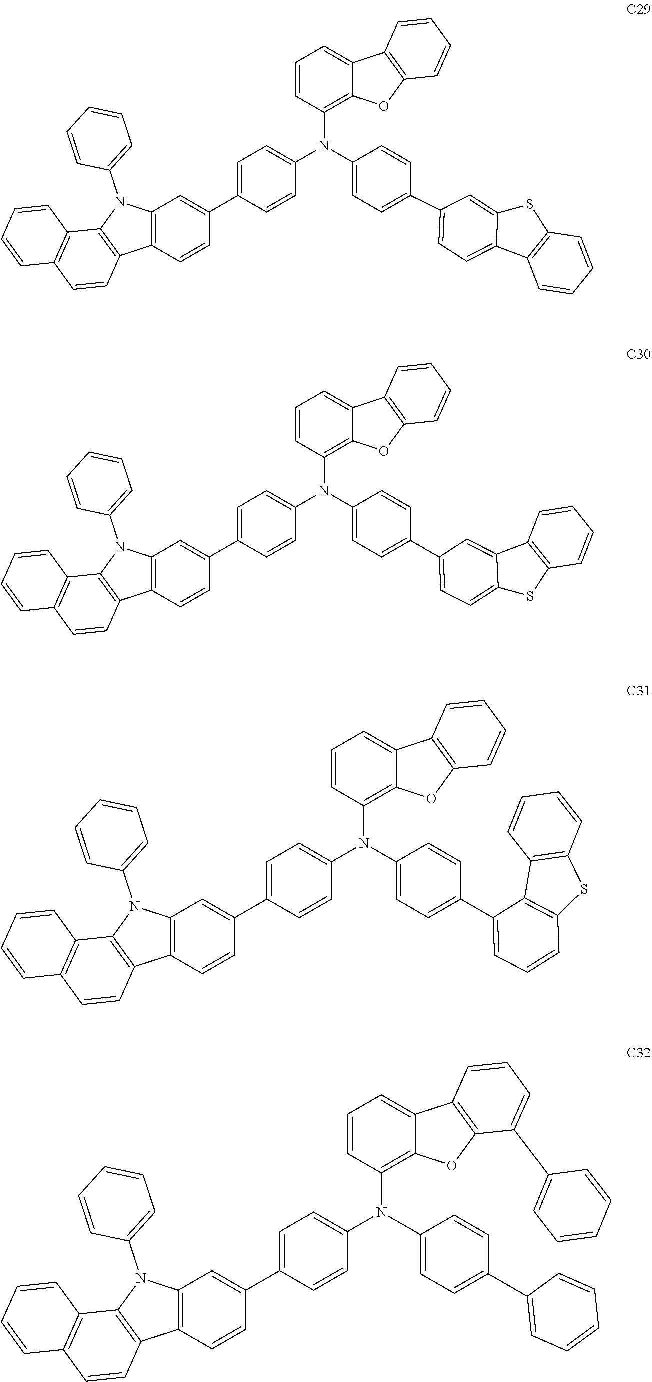 [SMILES: c1ccc(-n2c3cc(-c4ccc(N(c5ccc(-c6ccc7c(c6)sc6ccccc67)cc5)c5cccc6c5oc5ccccc56)cc4)ccc3c3ccc4ccccc4c32)cc1 ; c1ccc(-n2c3cc(-c4ccc(N(c5ccc(-c6ccc7sc8ccccc8c7c6)cc5)c5cccc6c5oc5ccccc56)cc4)ccc3c3ccc4ccccc4c32)cc1 ; c1ccc(-n2c3cc(-c4ccc(N(c5ccc(-c6cccc7sc8ccccc8c67)cc5)c5cccc6c5oc5ccccc56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3cccc4c3oc3c(-c5ccccc5)cccc34)cc2)cc1]

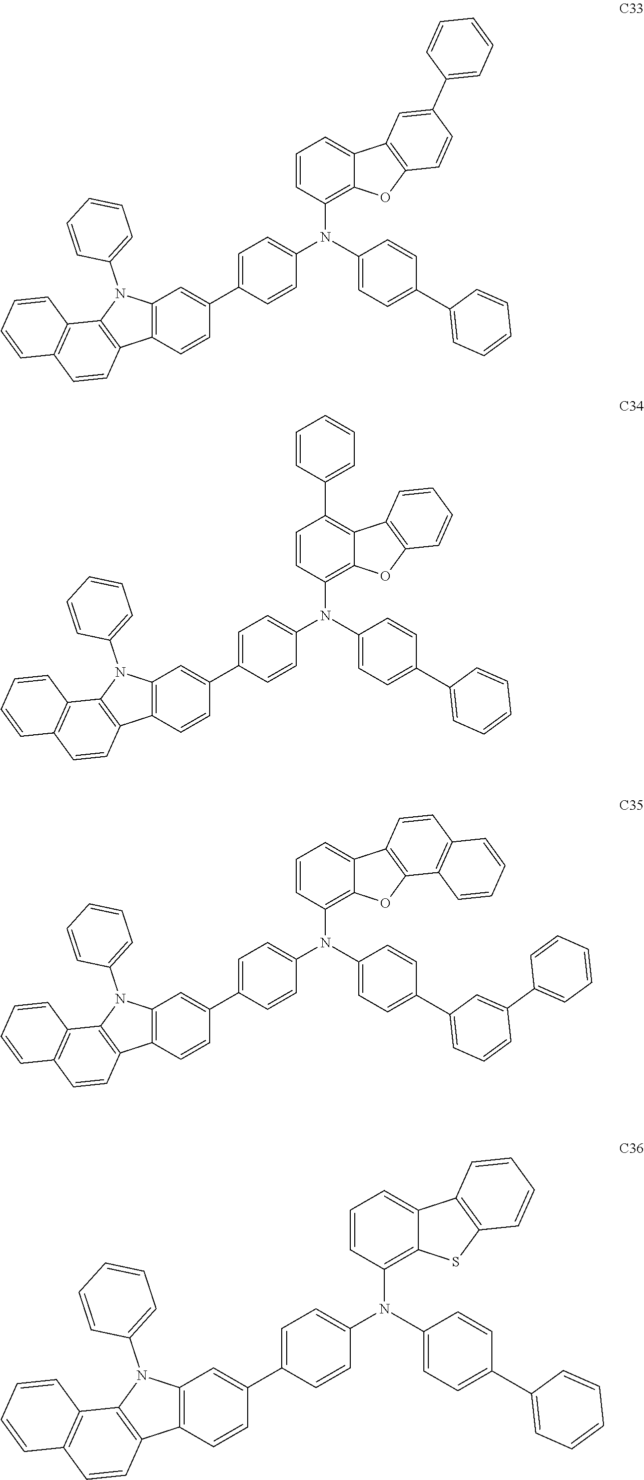 [SMILES: c1ccc(-c2ccc(N(c3ccc(-c4ccc5c6ccc7ccccc7c6n(-c6ccccc6)c5c4)cc3)c3cccc4c3oc3ccc(-c5ccccc5)cc34)cc2)cc1 ; c1ccc(-c2ccc(N(c3ccc(-c4ccc5c6ccc7ccccc7c6n(-c6ccccc6)c5c4)cc3)c3ccc(-c4ccccc4)c4c3oc3ccccc34)cc2)cc1 ; c1ccc(-c2cccc(-c3ccc(N(c4ccc(-c5ccc6c7ccc8ccccc8c7n(-c7ccccc7)c6c5)cc4)c4cccc5c4oc4c6ccccc6ccc54)cc3)c2)cc1 ; c1ccc(-c2ccc(N(c3ccc(-c4ccc5c6ccc7ccccc7c6n(-c6ccccc6)c5c4)cc3)c3cccc4c3sc3ccccc34)cc2)cc1]

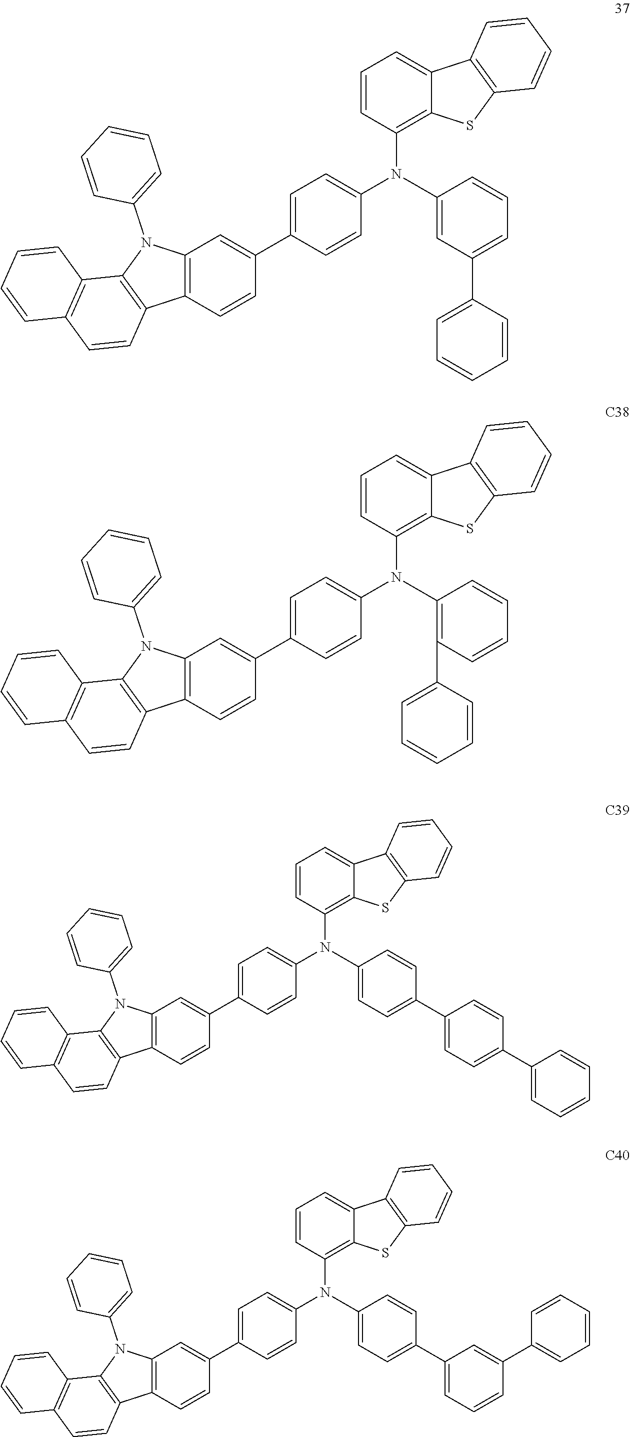 [SMILES: c1ccc(-c2cccc(N(c3ccc(-c4ccc5c6ccc7ccccc7c6n(-c6ccccc6)c5c4)cc3)c3cccc4c3sc3ccccc34)c2)cc1 ; c1ccc(-c2ccccc2N(c2ccc(-c3ccc4c5ccc6ccccc6c5n(-c5ccccc5)c4c3)cc2)c2cccc3c2sc2ccccc23)cc1 ; c1ccc(-c2ccc(-c3ccc(N(c4ccc(-c5ccc6c7ccc8ccccc8c7n(-c7ccccc7)c6c5)cc4)c4cccc5c4sc4ccccc45)cc3)cc2)cc1 ; c1ccc(-c2cccc(-c3ccc(N(c4ccc(-c5ccc6c7ccc8ccccc8c7n(-c7ccccc7)c6c5)cc4)c4cccc5c4sc4ccccc45)cc3)c2)cc1]

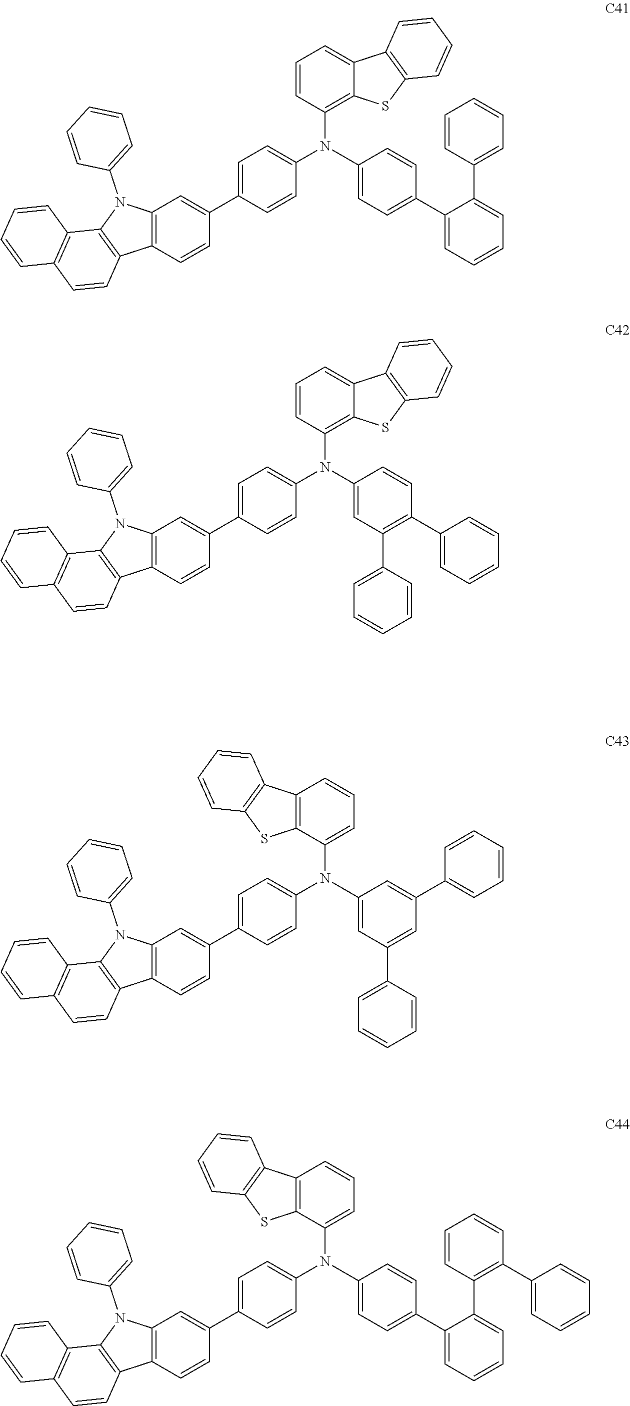 [SMILES: c1ccc(-c2ccccc2-c2ccc(N(c3ccc(-c4ccc5c6ccc7ccccc7c6n(-c6ccccc6)c5c4)cc3)c3cccc4c3sc3ccccc34)cc2)cc1 ; c1ccc(-c2ccc(N(c3ccc(-c4ccc5c6ccc7ccccc7c6n(-c6ccccc6)c5c4)cc3)c3cccc4c3sc3ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4c3sc3ccccc34)c2)cc1 ; c1ccc(-c2ccccc2-c2ccccc2-c2ccc(N(c3ccc(-c4ccc5c6ccc7ccccc7c6n(-c6ccccc6)c5c4)cc3)c3cccc4c3sc3ccccc34)cc2)cc1]

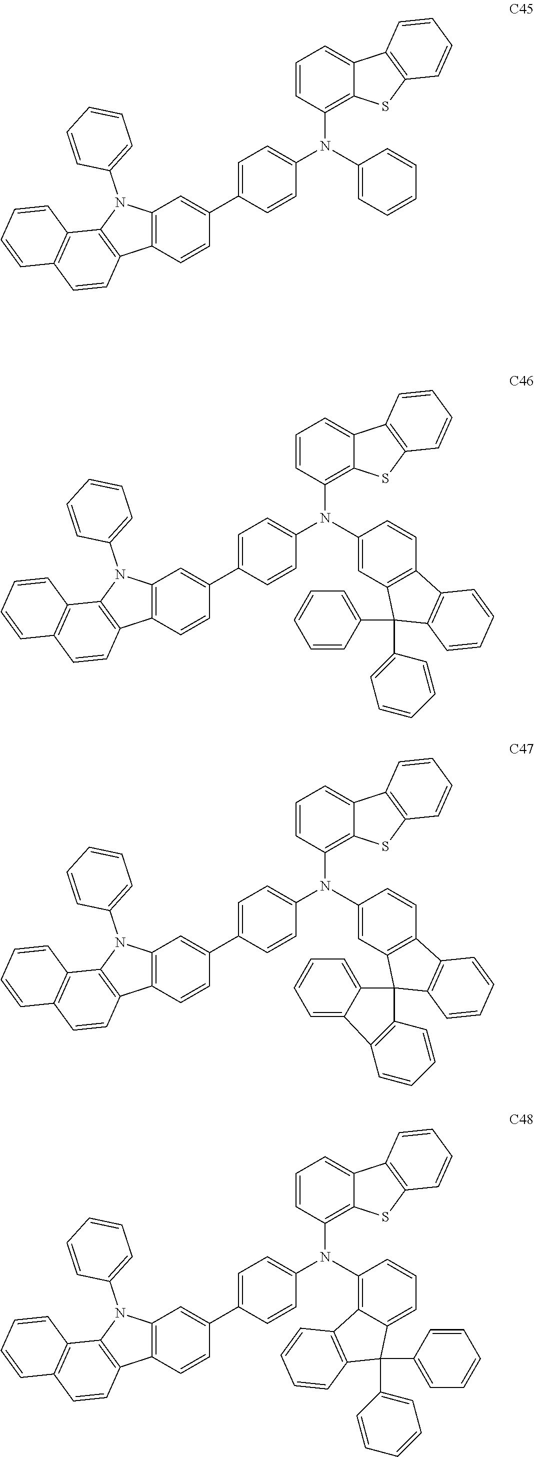

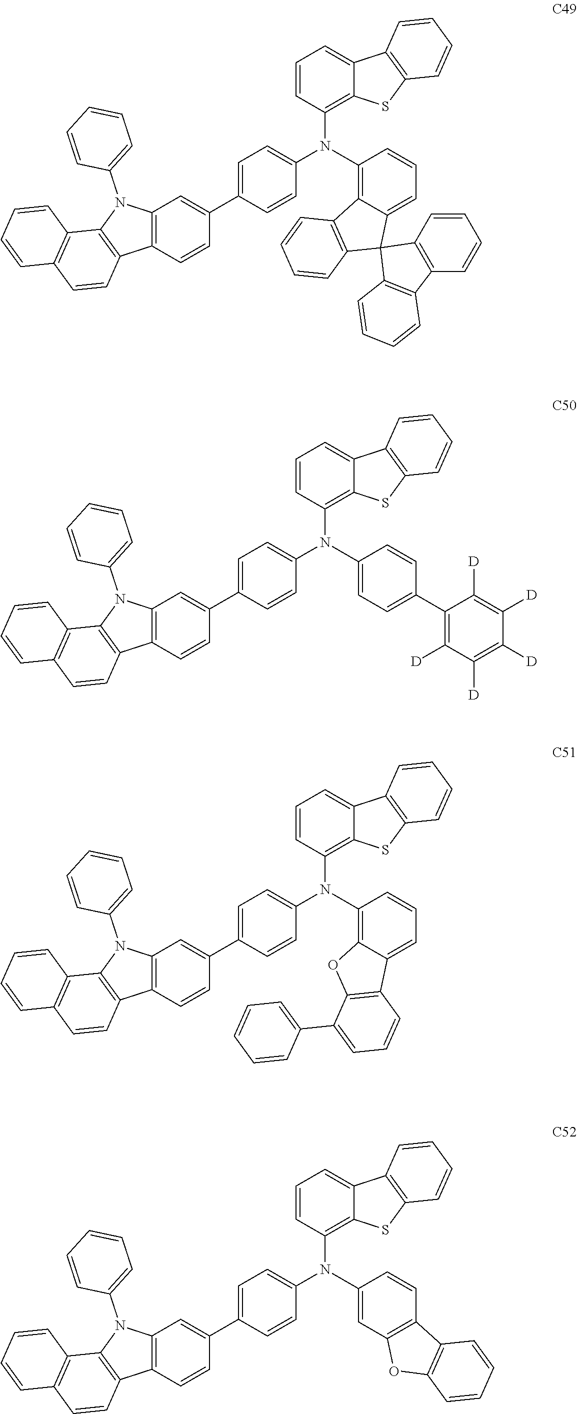 [SMILES: c1ccc(-n2c3cc(-c4ccc(N(c5cccc6c5-c5ccccc5C65c6ccccc6-c6ccccc65)c5cccc6c5sc5ccccc56)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3cccc4c3sc3ccccc34)cc2)c([2H])c1[2H] ; c1ccc(-c2cccc3c2oc2c(N(c4ccc(-c5ccc6c7ccc8ccccc8c7n(-c7ccccc7)c6c5)cc4)c4cccc5c4sc4ccccc45)cccc23)cc1 ; c1ccc(-n2c3cc(-c4ccc(N(c5ccc6c(c5)oc5ccccc56)c5cccc6c5sc5ccccc56)cc4)ccc3c3ccc4ccccc4c32)cc1]

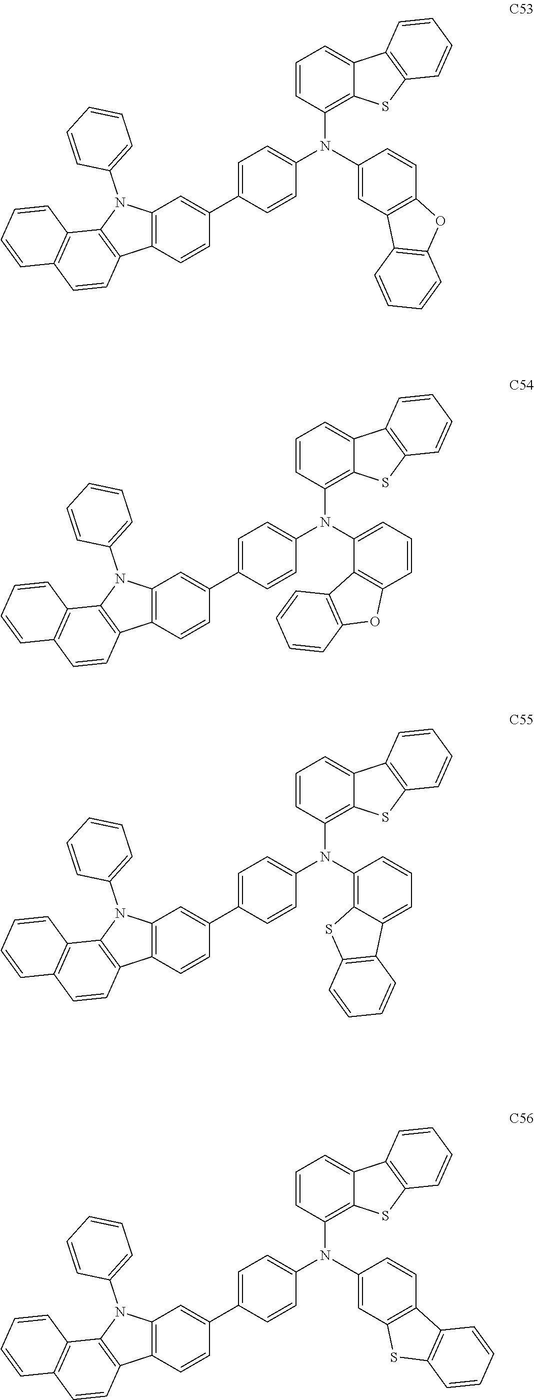 [SMILES: c1ccc(-n2c3cc(-c4ccc(N(c5ccc6oc7ccccc7c6c5)c5cccc6c5sc5ccccc56)cc4)ccc3c3ccc4ccccc4c32)cc1 ; c1ccc(-n2c3cc(-c4ccc(N(c5cccc6c5sc5ccccc56)c5cccc6oc7ccccc7c56)cc4)ccc3c3ccc4ccccc4c32)cc1 ; c1ccc(-n2c3cc(-c4ccc(N(c5cccc6c5sc5ccccc56)c5cccc6c5sc5ccccc56)cc4)ccc3c3ccc4ccccc4c32)cc1 ; c1ccc(-n2c3cc(-c4ccc(N(c5ccc6c(c5)sc5ccccc56)c5cccc6c5sc5ccccc56)cc4)ccc3c3ccc4ccccc4c32)cc1]

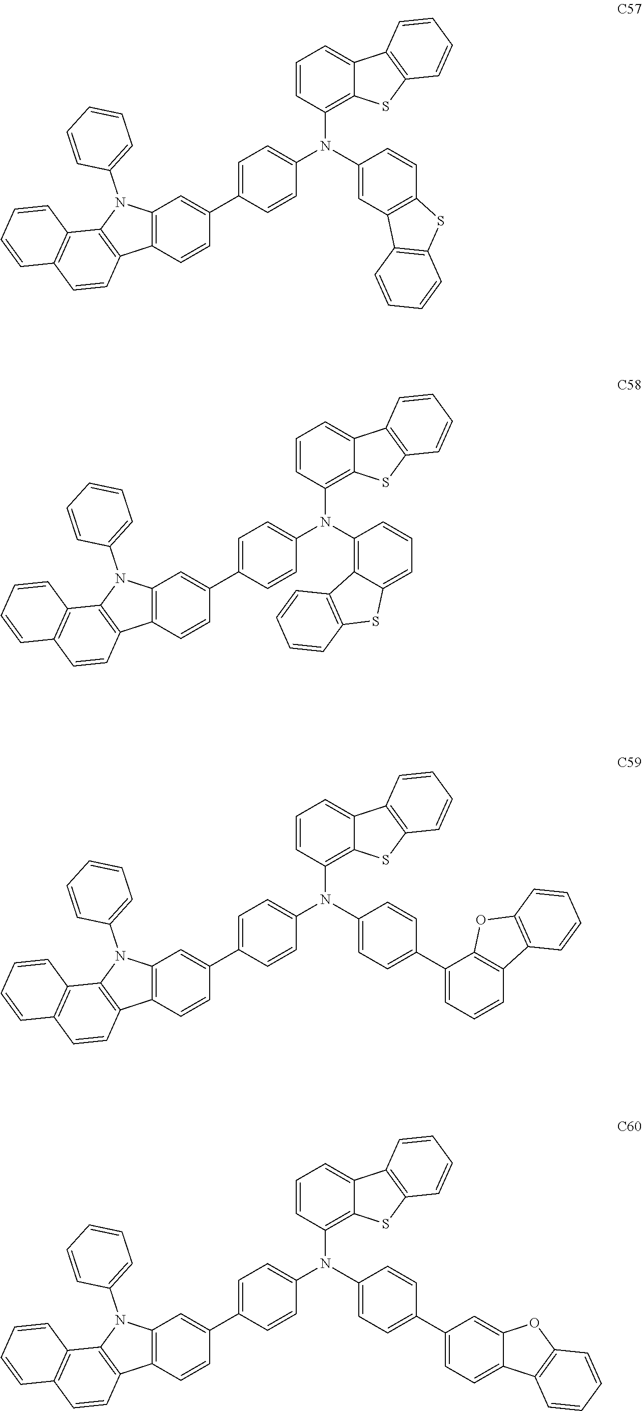 [SMILES: c1ccc(-n2c3cc(-c4ccc(N(c5ccc6sc7ccccc7c6c5)c5cccc6c5sc5ccccc56)cc4)ccc3c3ccc4ccccc4c32)cc1 ; c1ccc(-n2c3cc(-c4ccc(N(c5cccc6c5sc5ccccc56)c5cccc6sc7ccccc7c56)cc4)ccc3c3ccc4ccccc4c32)cc1 ; c1ccc(-n2c3cc(-c4ccc(N(c5ccc(-c6cccc7c6oc6ccccc67)cc5)c5cccc6c5sc5ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5cccc6c5sc5ccccc56)cc4)ccc3c3ccc4ccccc4c32)cc1]

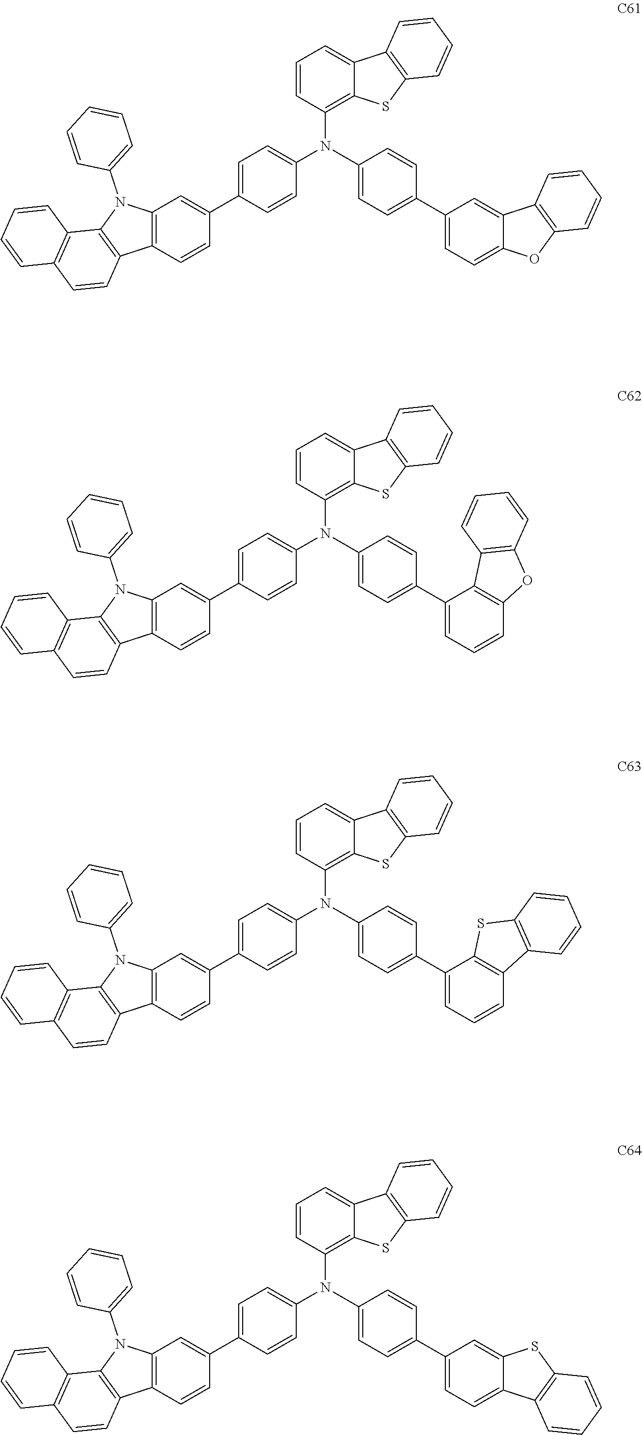 [SMILES: c1ccc(-n2c3cc(-c4ccc(N(c5ccc(-c6ccc7oc8ccccc8c7c6)cc5)c5cccc6c5sc5ccccc56)cc4)ccc3c3ccc4ccccc4c32)cc1 ; c1ccc(-n2c3cc(-c4ccc(N(c5ccc(-c6cccc7oc8ccccc8c67)cc5)c5cccc6c5sc5ccccc56)cc4)ccc3c3ccc4ccccc4c32)cc1 ; c1ccc(-n2c3cc(-c4ccc(N(c5ccc(-c6cccc7c6sc6ccccc67)cc5)c5cccc6c5sc5ccccc56)cc4)ccc3c3ccc4ccccc4c32)cc1 ; c1ccc(-n2c3cc(-c4ccc(N(c5ccc(-c6ccc7c(c6)sc6ccccc67)cc5)c5cccc6c5sc5ccccc56)cc4)ccc3c3ccc4ccccc4c32)cc1]

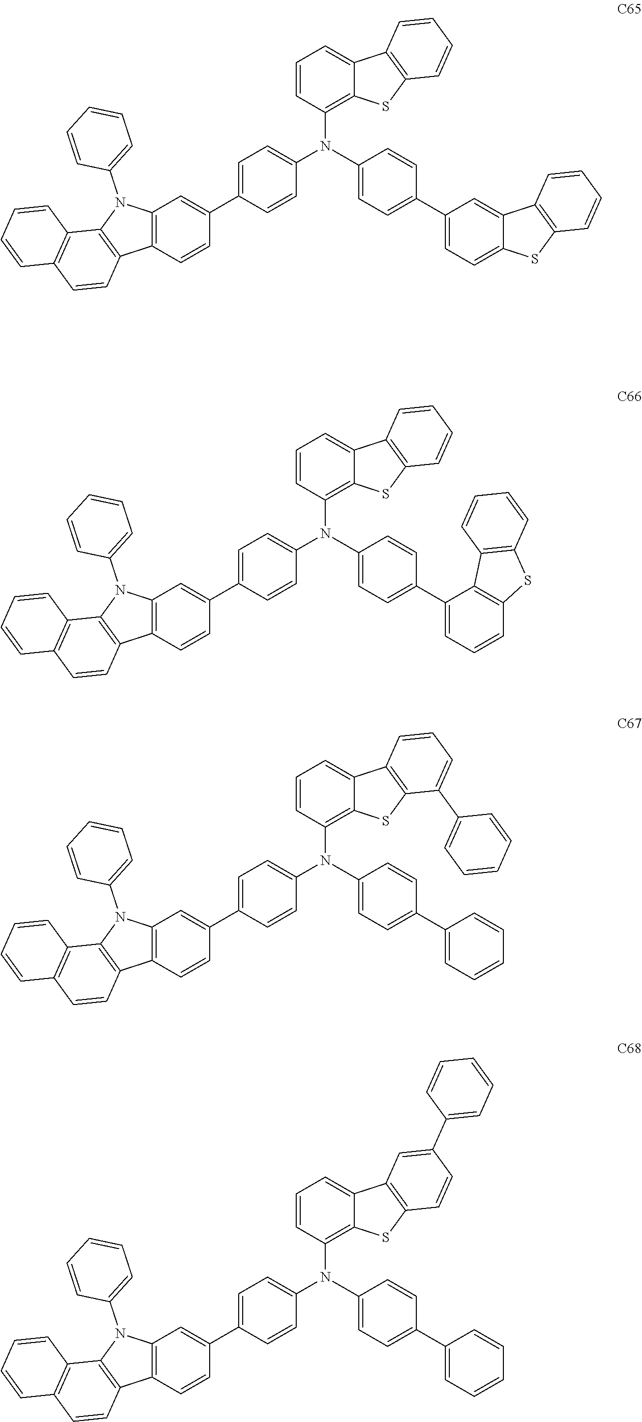 [SMILES: c1ccc(-n2c3cc(-c4ccc(N(c5ccc(-c6ccc7sc8ccccc8c7c6)cc5)c5cccc6c5sc5ccccc56)cc4)ccc3c3ccc4ccccc4c32)cc1 ; c1ccc(-n2c3cc(-c4ccc(N(c5ccc(-c6cccc7sc8ccccc8c67)cc5)c5cccc6c5sc5ccccc56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3cccc4c3sc3c(-c5ccccc5)cccc34)cc2)cc1 ; c1ccc(-c2ccc(N(c3ccc(-c4ccc5c6ccc7ccccc7c6n(-c6ccccc6)c5c4)cc3)c3cccc4c3sc3ccc(-c5ccccc5)cc34)cc2)cc1]

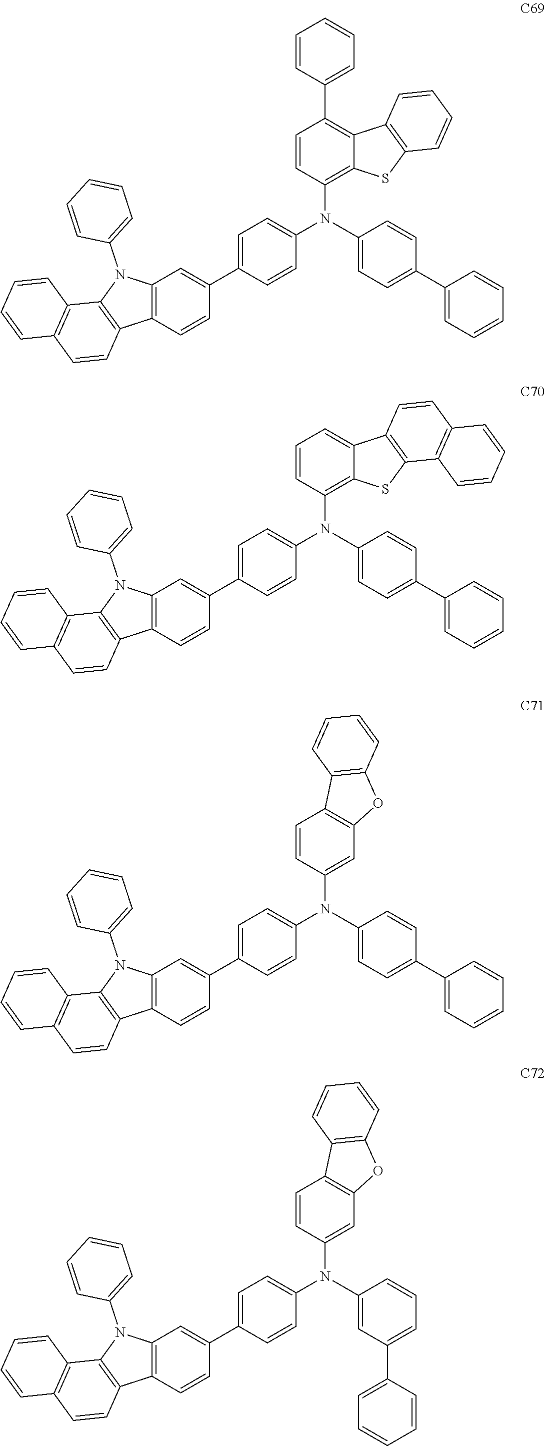 [SMILES: c1ccc(-c2ccc(N(c3ccc(-c4ccc5c6ccc7ccccc7c6n(-c6ccccc6)c5c4)cc3)c3ccc(-c4ccccc4)c4c3sc3ccccc34)cc2)cc1 ; c1ccc(-c2ccc(N(c3ccc(-c4ccc5c6ccc7ccccc7c6n(-c6ccccc6)c5c4)cc3)c3cccc4c3sc3c5ccccc5ccc43)cc2)cc1 ; c1ccc(-c2ccc(N(c3ccc(-c4ccc5c6ccc7ccccc7c6n(-c6ccccc6)c5c4)cc3)c3ccc4c(c3)oc3ccccc34)cc2)cc1 ; c1ccc(-c2cccc(N(c3ccc(-c4ccc5c6ccc7ccccc7c6n(-c6ccccc6)c5c4)cc3)c3ccc4c(c3)oc3ccccc34)c2)cc1]

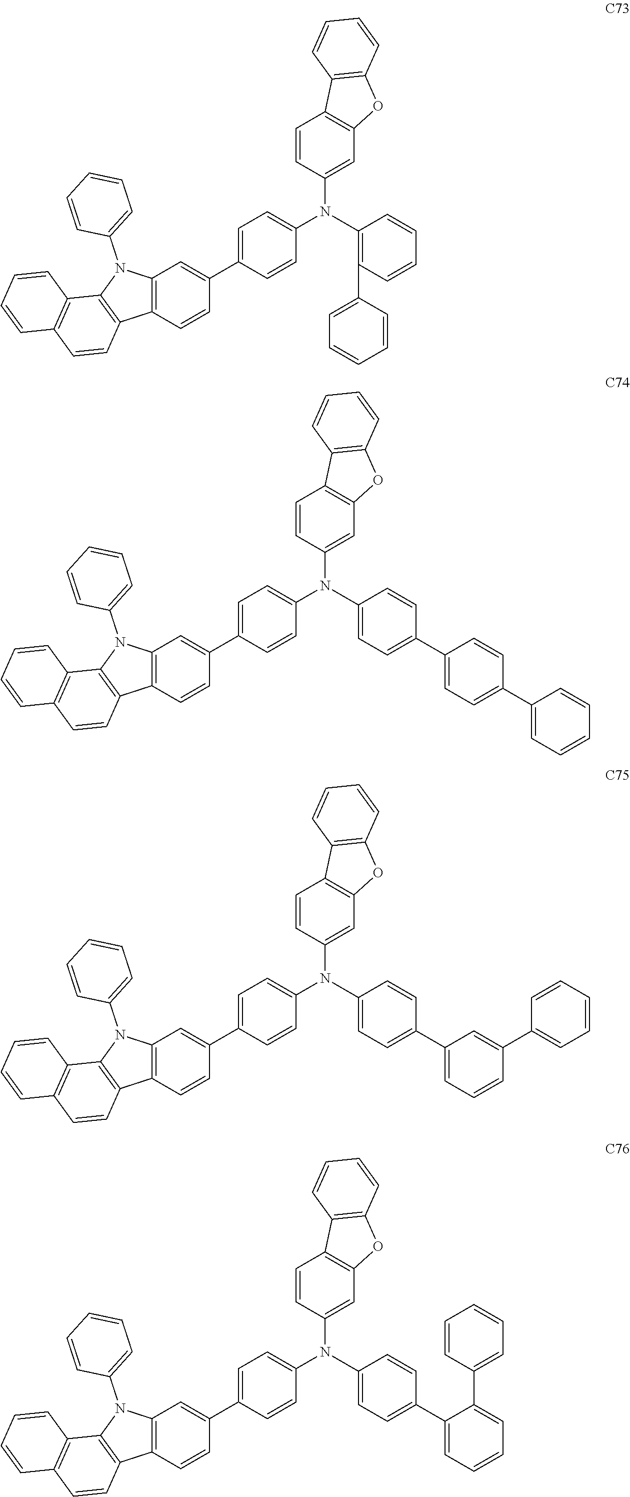

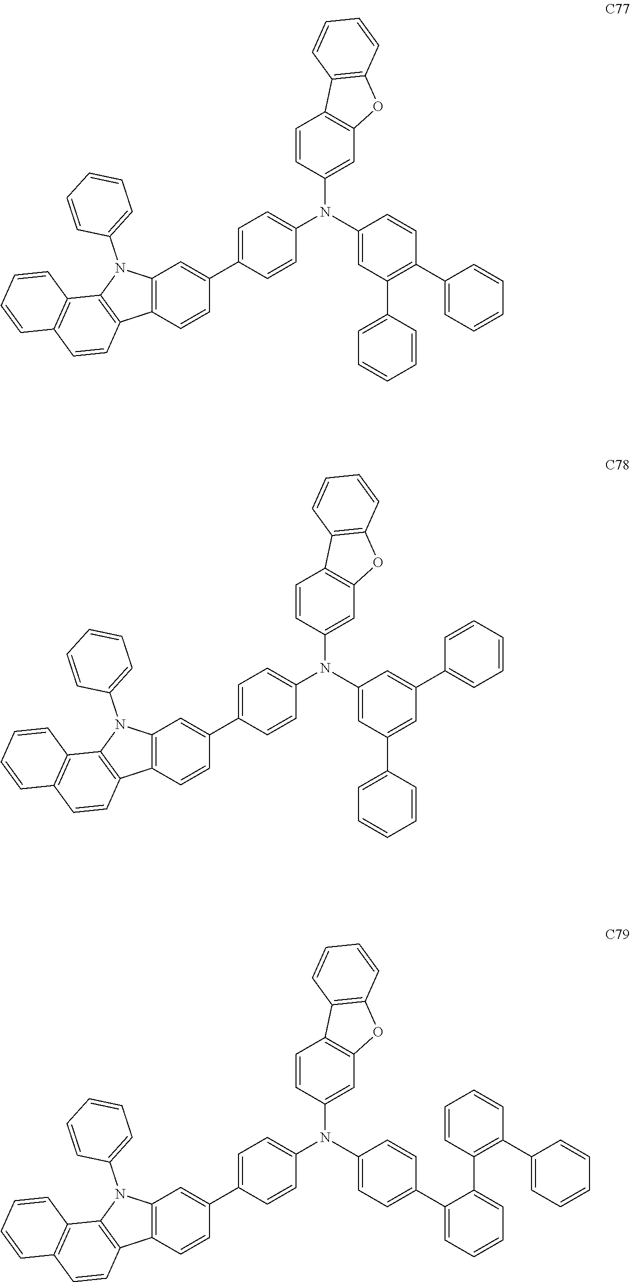 [SMILES: c1ccc(-c2ccc(N(c3ccc(-c4ccc5c6ccc7ccccc7c6n(-c6ccccc6)c5c4)cc3)c3ccc4c(c3)oc3ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(c3)oc3ccccc34)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)oc3ccccc34)cc2)cc1]

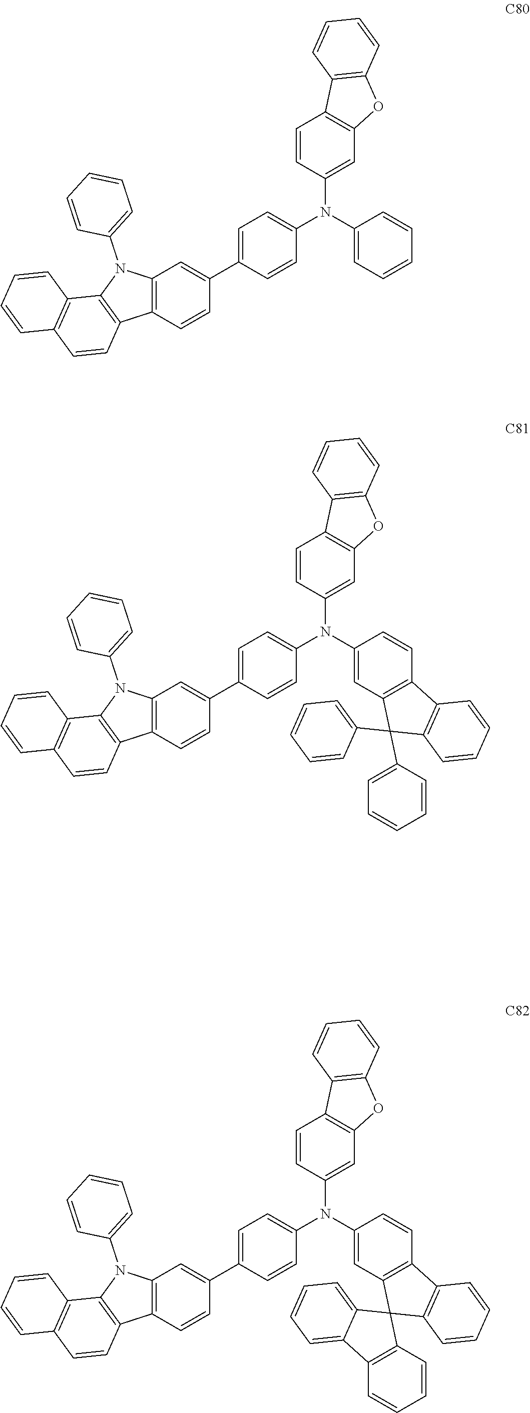 [SMILES: c1ccc(N(c2ccc(-c3ccc4c5ccc6ccccc6c5n(-c5ccccc5)c4c3)cc2)c2ccc3c(c2)oc2ccccc23)cc1 ; c1ccc(-n2c3cc(-c4ccc(N(c5ccc6c(c5)C(c5ccccc5)(c5ccccc5)c5ccccc5-6)c5ccc6c(c5)oc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6c(c5)oc5ccccc56)cc4)ccc3c3ccc4ccccc4c32)cc1]

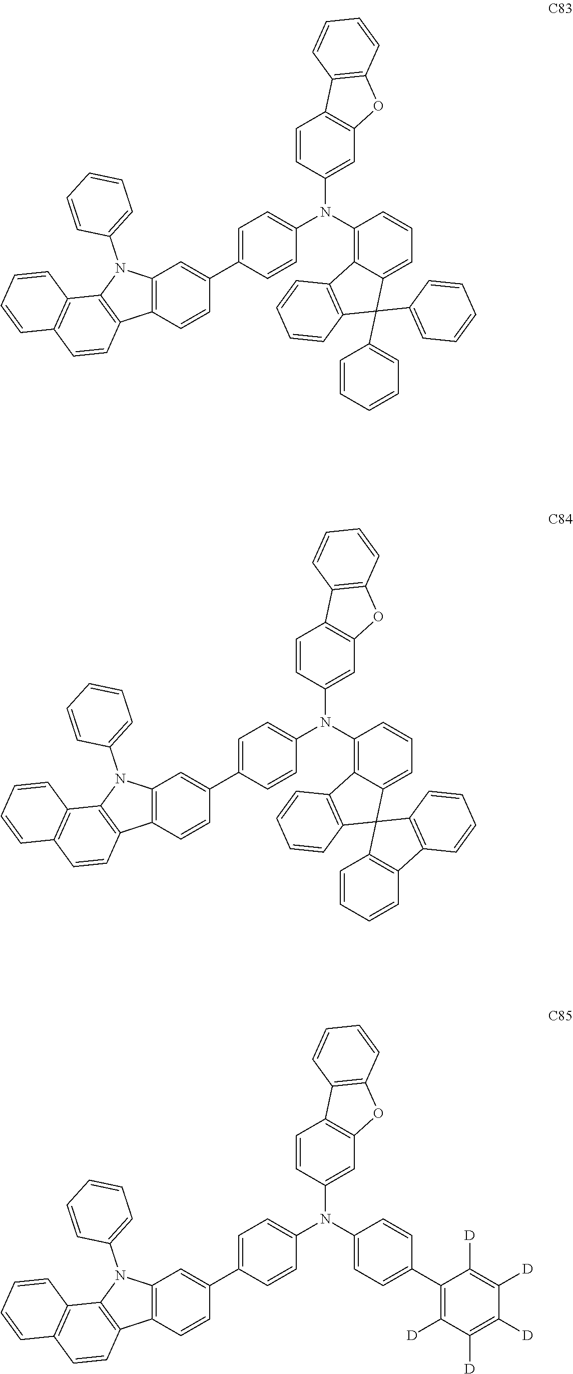 [SMILES: c1ccc(-n2c3cc(-c4ccc(N(c5ccc6c(c5)oc5ccccc56)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc6c(c5)oc5ccccc56)c5cccc6c5-c5ccccc5C65c6ccccc6-c6ccccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4c(c3)oc3ccccc34)cc2)c([2H])c1[2H]]

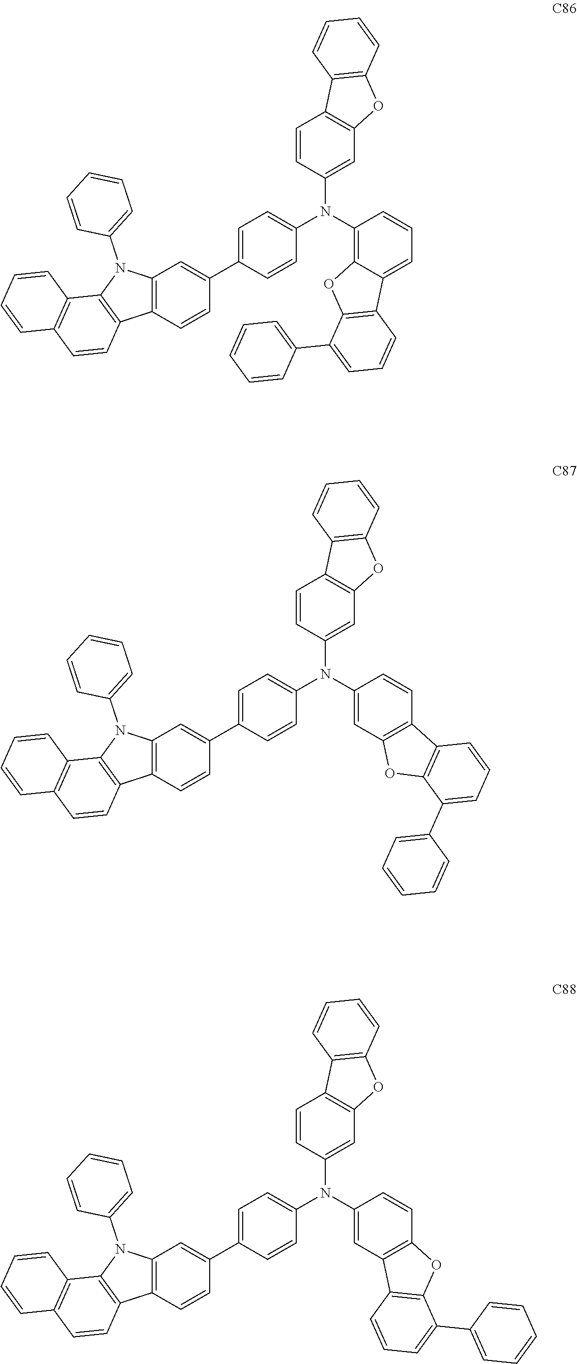 [SMILES: c1ccc(-c2cccc3c2oc2c(N(c4ccc(-c5ccc6c7ccc8ccccc8c7n(-c7ccccc7)c6c5)cc4)c4ccc5c(c4)oc4ccccc45)cccc23)cc1 ; c1ccc(-c2cccc3c2oc2cc(N(c4ccc(-c5ccc6c7ccc8ccccc8c7n(-c7ccccc7)c6c5)cc4)c4ccc5c(c4)oc4ccccc45)ccc23)cc1 ; c1ccc(-c2cccc3c2oc2ccc(N(c4ccc(-c5ccc6c7ccc8ccccc8c7n(-c7ccccc7)c6c5)cc4)c4ccc5c(c4)oc4ccccc45)cc23)cc1]

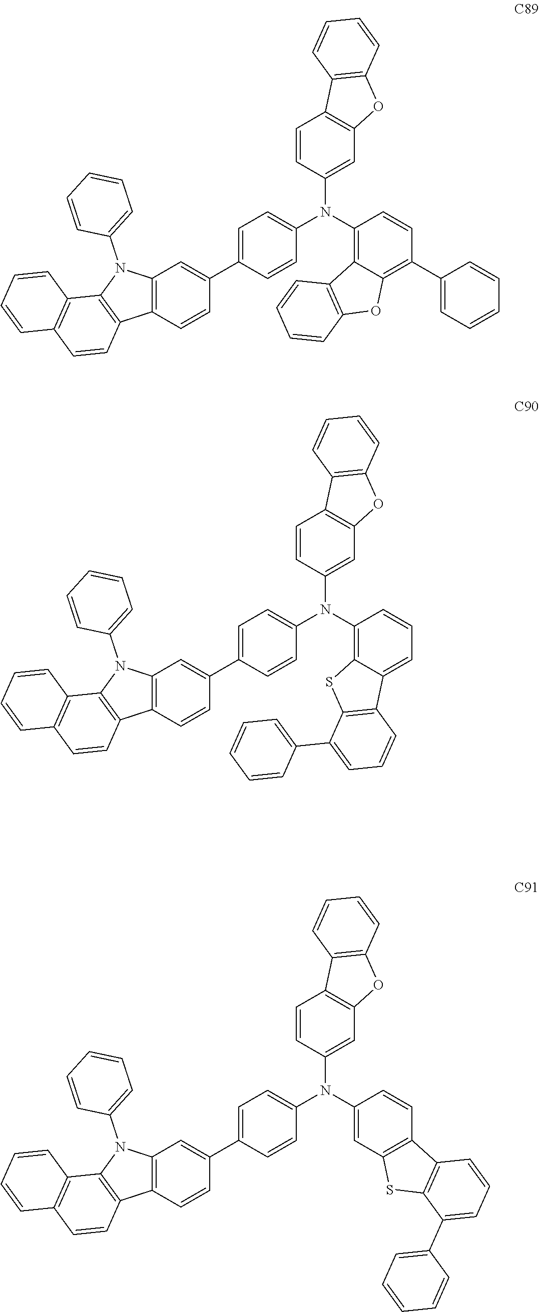 [SMILES: c1ccc(-c2ccc(N(c3ccc(-c4ccc5c6ccc7ccccc7c6n(-c6ccccc6)c5c4)cc3)c3ccc4c(c3)oc3ccccc34)c3c2oc2ccccc23)cc1 ; c1ccc(-c2cccc3c2sc2c(N(c4ccc(-c5ccc6c7ccc8ccccc8c7n(-c7ccccc7)c6c5)cc4)c4ccc5c(c4)oc4ccccc45)cccc23)cc1 ; c1ccc(-c2cccc3c2sc2cc(N(c4ccc(-c5ccc6c7ccc8ccccc8c7n(-c7ccccc7)c6c5)cc4)c4ccc5c(c4)oc4ccccc45)ccc23)cc1]

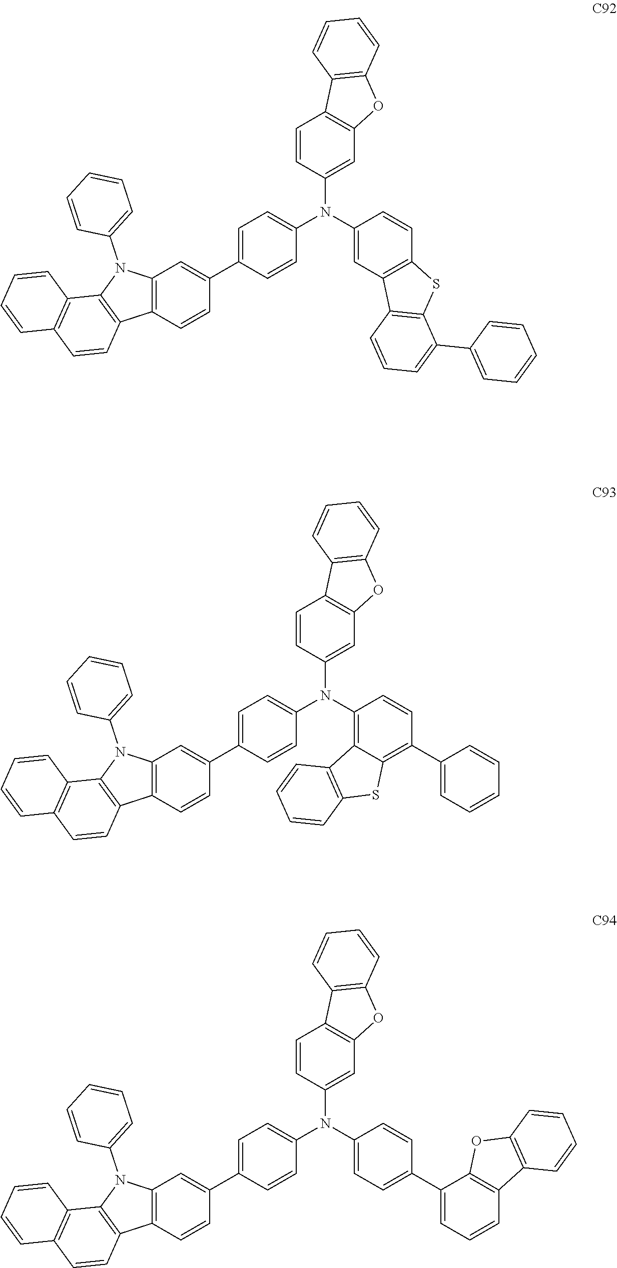 [SMILES: c1ccc(-c2cccc3c2sc2ccc(N(c4ccc(-c5ccc6c7ccc8ccccc8c7n(-c7ccccc7)c6c5)cc4)c4ccc5c(c4)oc4ccccc45)cc23)cc1 ; c1ccc(-c2ccc(N(c3ccc(-c4ccc5c6ccc7ccccc7c6n(-c6ccccc6)c5c4)cc3)c3ccc4c(c3)oc3ccccc34)c3c2sc2ccccc23)cc1 ; c1ccc(-n2c3cc(-c4ccc(N(c5ccc(-c6cccc7c6oc6ccccc67)cc5)c5ccc6c(c5)oc5ccccc56)cc4)ccc3c3ccc4ccccc4c32)cc1]

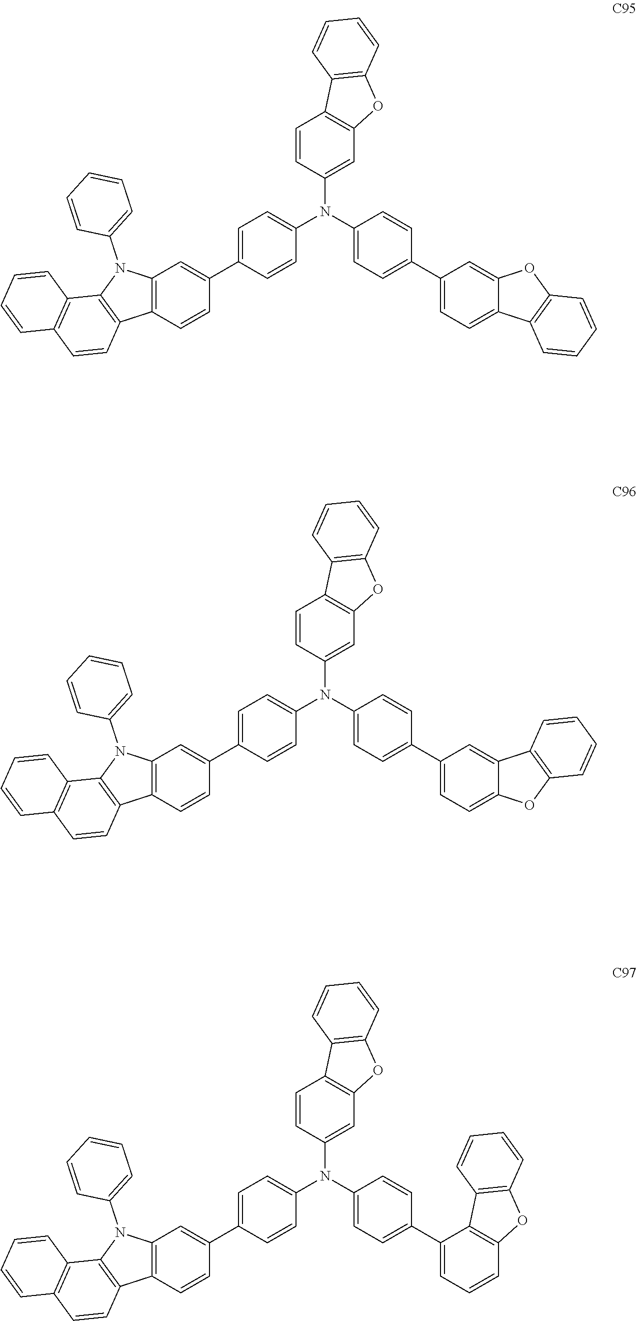 [SMILES: c1ccc(-n2c3cc(-c4ccc(N(c5ccc(-c6ccc7c(c6)oc6ccccc67)cc5)c5ccc6c(c5)oc5ccccc56)cc4)ccc3c3ccc4ccccc4c32)cc1 ; c1ccc(-n2c3cc(-c4ccc(N(c5ccc(-c6ccc7oc8ccccc8c7c6)cc5)c5ccc6c(c5)oc5ccccc56)cc4)ccc3c3ccc4ccccc4c32)cc1 ; c1ccc(-n2c3cc(-c4ccc(N(c5ccc(-c6cccc7oc8ccccc8c67)cc5)c5ccc6c(c5)oc5ccccc56)cc4)ccc3c3ccc4ccccc4c32)cc1]

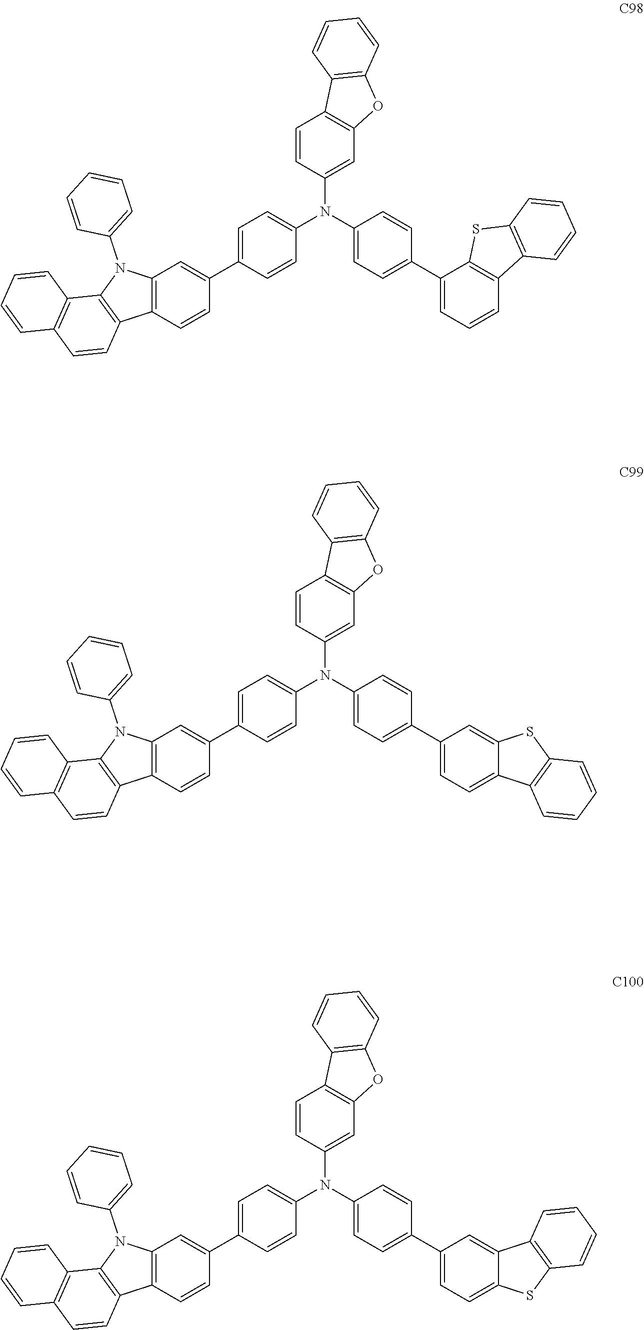 [SMILES: c1ccc(-n2c3cc(-c4ccc(N(c5ccc(-c6cccc7c6sc6ccccc67)cc5)c5ccc6c(c5)oc5ccccc56)cc4)ccc3c3ccc4ccccc4c32)cc1 ; c1ccc(-n2c3cc(-c4ccc(N(c5ccc(-c6ccc7c(c6)sc6ccccc67)cc5)c5ccc6c(c5)oc5ccccc56)cc4)ccc3c3ccc4ccccc4c32)cc1 ; c1ccc(-n2c3cc(-c4ccc(N(c5ccc(-c6ccc7sc8ccccc8c7c6)cc5)c5ccc6c(c5)oc5ccccc56)cc4)ccc3c3ccc4ccccc4c32)cc1]

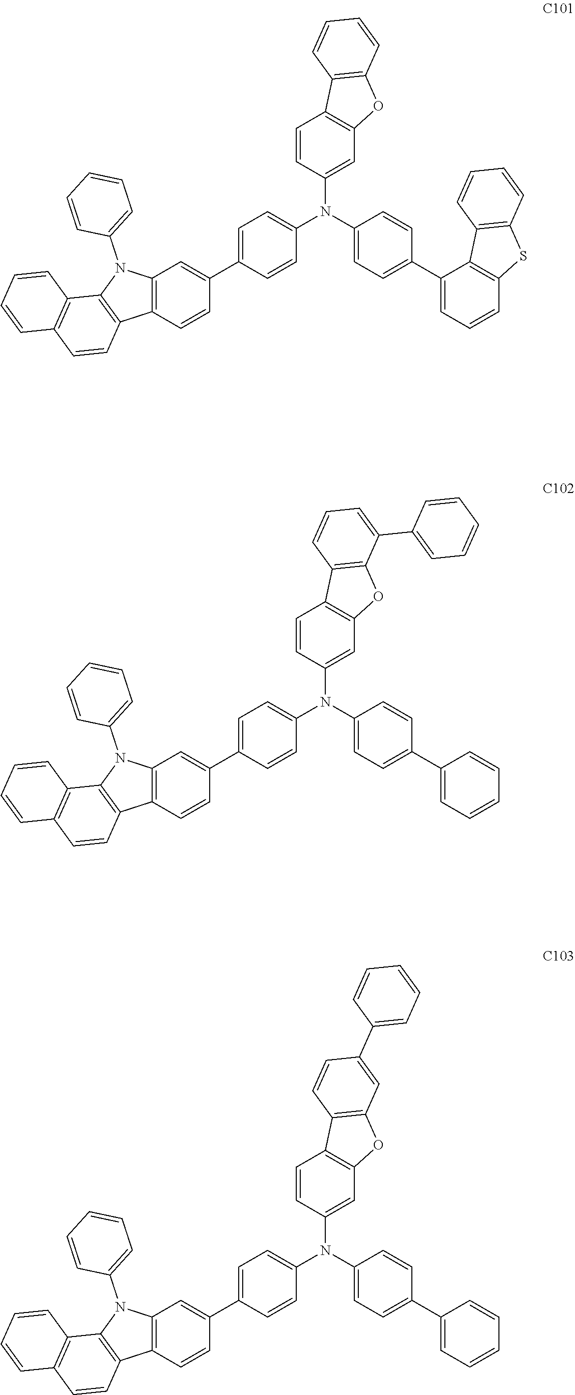 [SMILES: c1ccc(-n2c3cc(-c4ccc(N(c5ccc(-c6cccc7sc8ccccc8c67)cc5)c5ccc6c(c5)oc5ccccc56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4c(c3)oc3c(-c5ccccc5)cccc34)cc2)cc1 ; c1ccc(-c2ccc(N(c3ccc(-c4ccc5c6ccc7ccccc7c6n(-c6ccccc6)c5c4)cc3)c3ccc4c(c3)oc3cc(-c5ccccc5)ccc34)cc2)cc1]

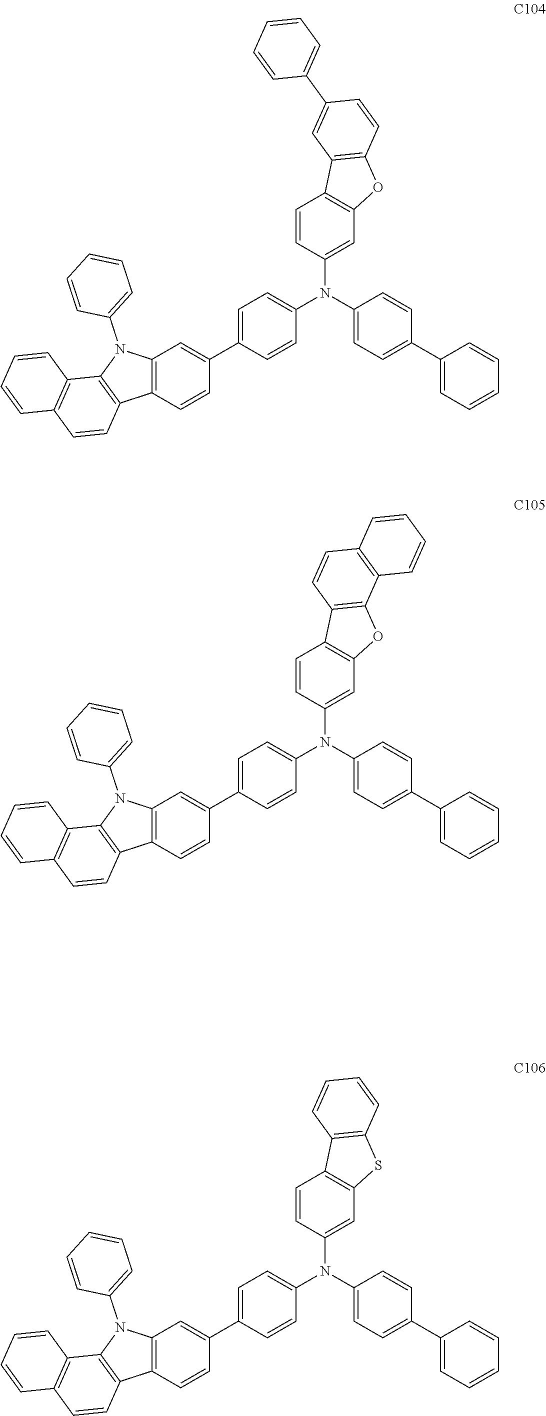 [SMILES: c1ccc(-c2ccc(N(c3ccc(-c4ccc5c6ccc7ccccc7c6n(-c6ccccc6)c5c4)cc3)c3ccc4c(c3)oc3ccc(-c5ccccc5)cc34)cc2)cc1 ; c1ccc(-c2ccc(N(c3ccc(-c4ccc5c6ccc7ccccc7c6n(-c6ccccc6)c5c4)cc3)c3ccc4c(c3)oc3c5ccccc5ccc43)cc2)cc1 ; c1ccc(-c2ccc(N(c3ccc(-c4ccc5c6ccc7ccccc7c6n(-c6ccccc6)c5c4)cc3)c3ccc4c(c3)sc3ccccc34)cc2)cc1]

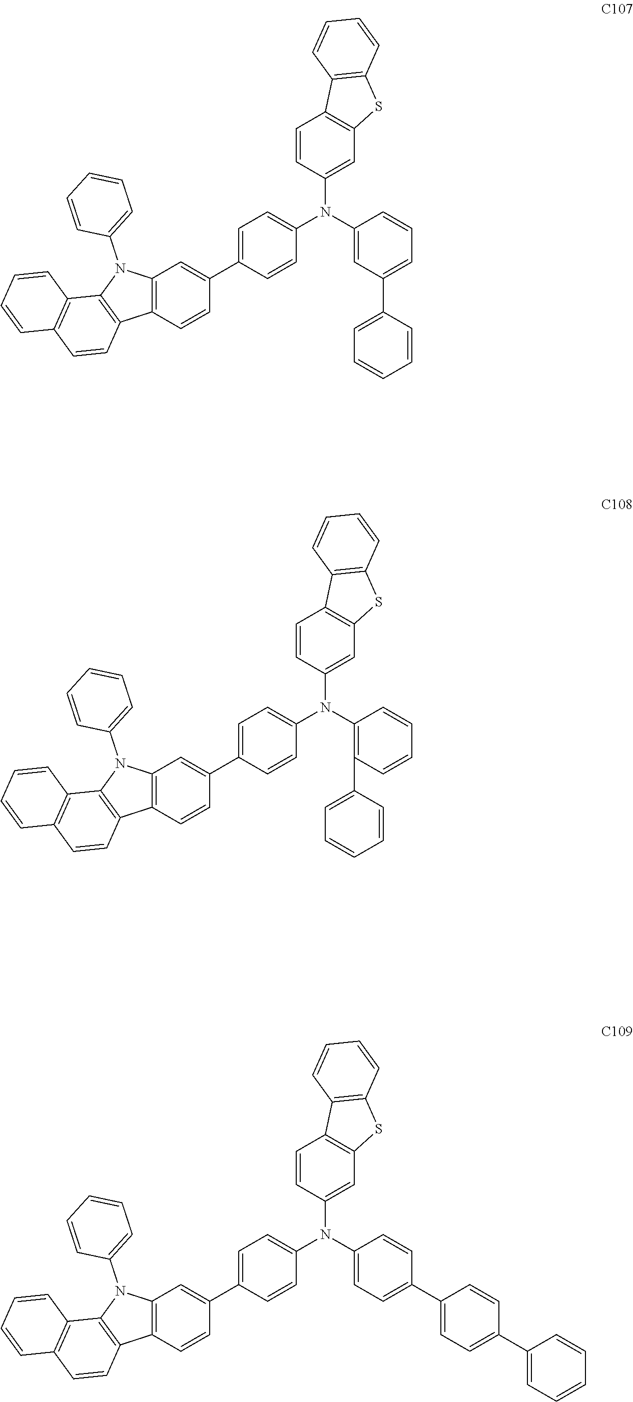 [SMILES: c1ccc(-c2cccc(N(c3ccc(-c4ccc5c6ccc7ccccc7c6n(-c6ccccc6)c5c4)cc3)c3ccc4c(c3)sc3ccccc34)c2)cc1 ; c1ccc(-c2ccccc2N(c2ccc(-c3ccc4c5ccc6ccccc6c5n(-c5ccccc5)c4c3)cc2)c2ccc3c(c2)sc2ccccc23)cc1 ; c1ccc(-c2ccc(-c3ccc(N(c4ccc(-c5ccc6c7ccc8ccccc8c7n(-c7ccccc7)c6c5)cc4)c4ccc5c(c4)sc4ccccc45)cc3)cc2)cc1]

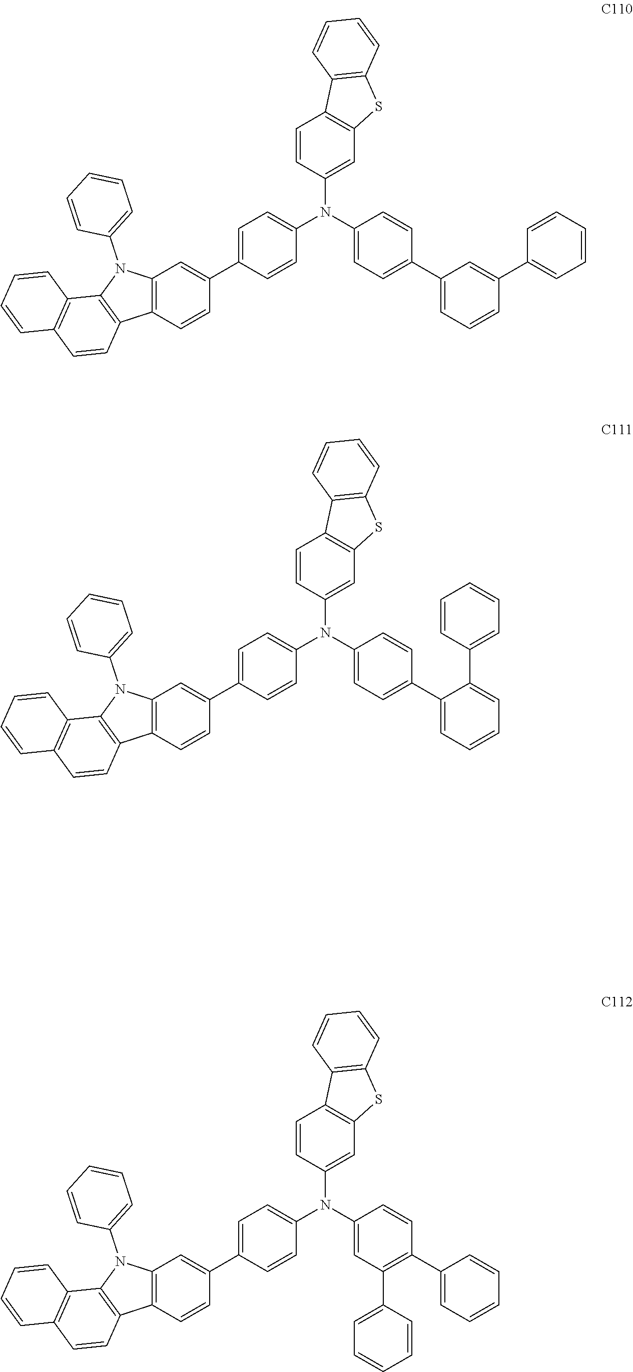 [SMILES: c1ccc(-c2cccc(-c3ccc(N(c4ccc(-c5ccc6c7ccc8ccccc8c7n(-c7ccccc7)c6c5)cc4)c4ccc5c(c4)sc4ccccc45)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(c3)sc3ccccc34)cc2)cc1 ; c1ccc(-c2ccc(N(c3ccc(-c4ccc5c6ccc7ccccc7c6n(-c6ccccc6)c5c4)cc3)c3ccc4c(c3)sc3ccccc34)cc2-c2ccccc2)cc1]

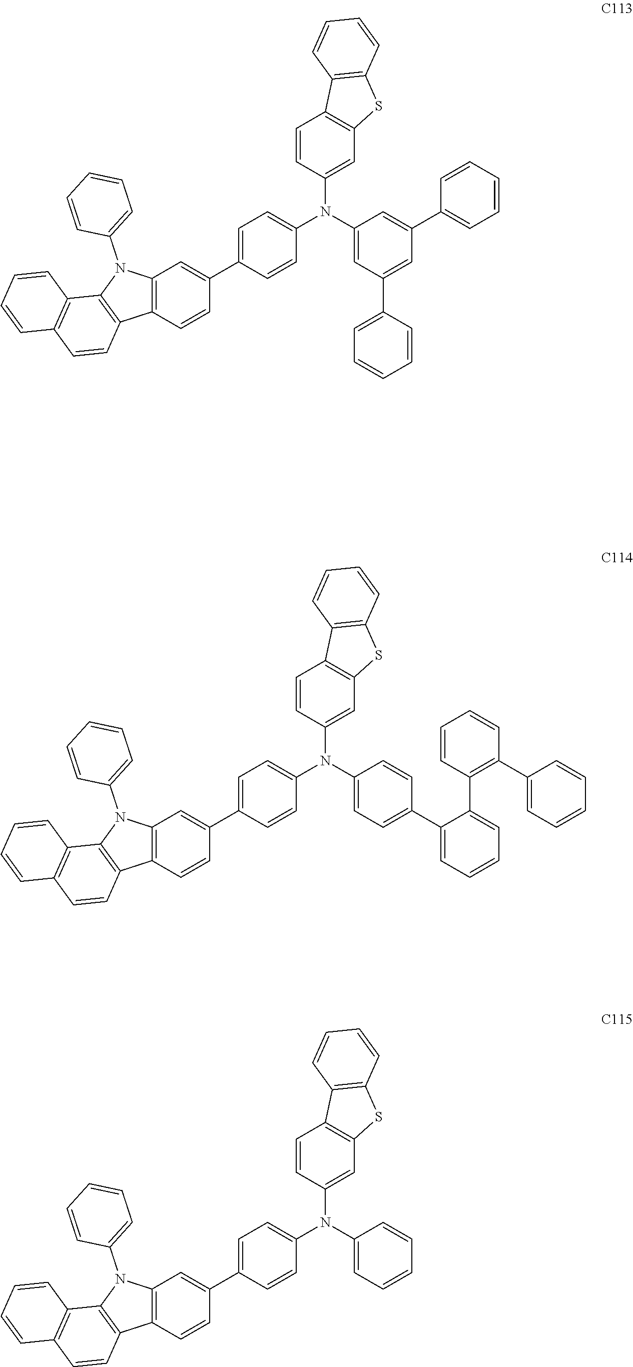 [SMILES: c1ccc(-c2cc(-c3ccccc3)cc(N(c3ccc(-c4ccc5c6ccc7ccccc7c6n(-c6ccccc6)c5c4)cc3)c3ccc4c(c3)sc3ccccc34)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)sc3ccccc34)cc2)cc1 ; c1ccc(N(c2ccc(-c3ccc4c5ccc6ccccc6c5n(-c5ccccc5)c4c3)cc2)c2ccc3c(c2)sc2ccccc23)cc1]

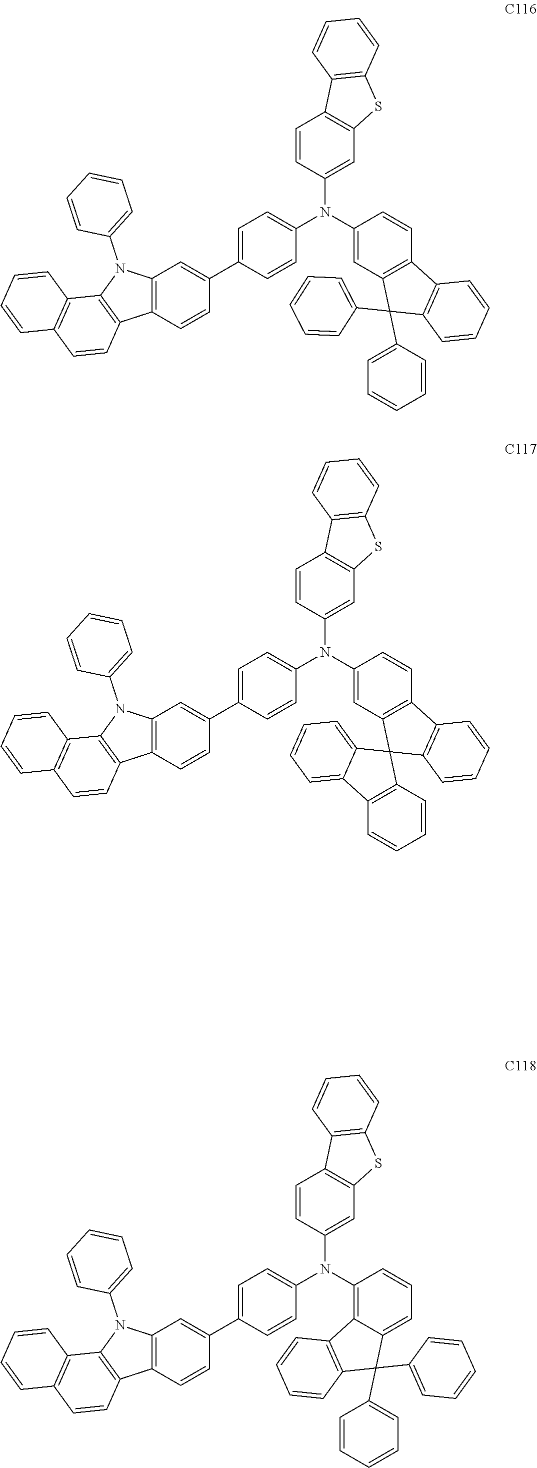 [SMILES: c1ccc(-n2c3cc(-c4ccc(N(c5ccc6c(c5)C(c5ccccc5)(c5ccccc5)c5ccccc5-6)c5ccc6c(c5)sc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6c(c5)sc5ccccc56)cc4)ccc3c3ccc4ccccc4c32)cc1 ; c1ccc(-n2c3cc(-c4ccc(N(c5ccc6c(c5)sc5ccccc56)c5cccc6c5-c5ccccc5C6(c5ccccc5)c5ccccc5)cc4)ccc3c3ccc4ccccc4c32)cc1]

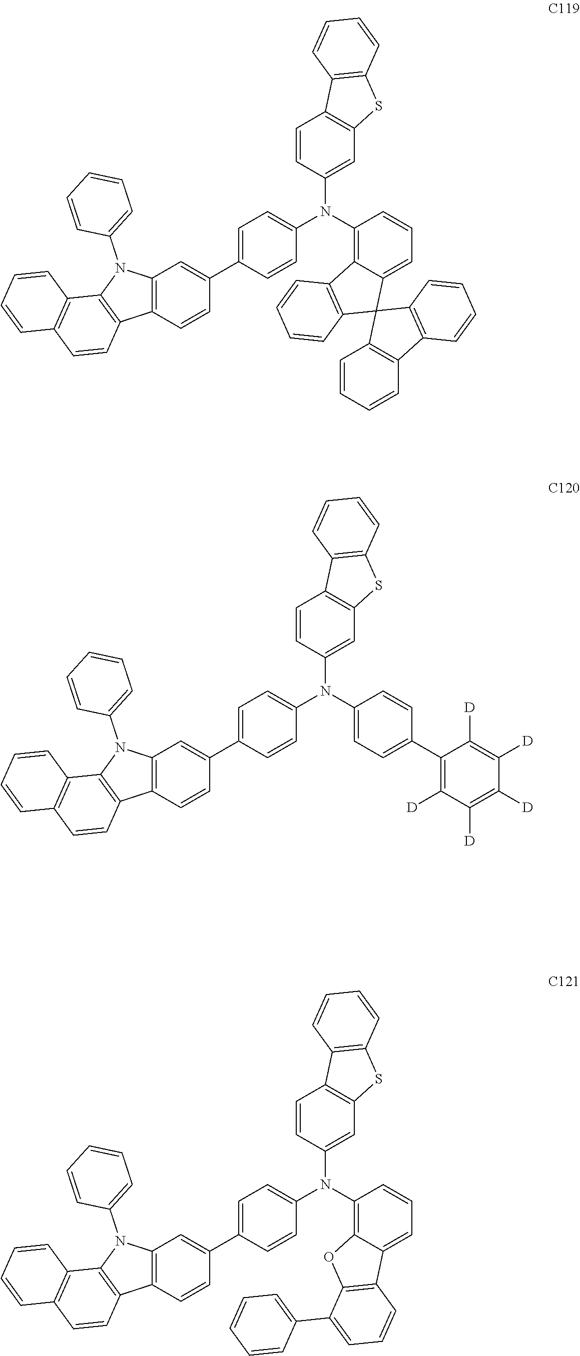 [SMILES: c1ccc(-n2c3cc(-c4ccc(N(c5ccc6c(c5)sc5ccccc56)c5cccc6c5-c5ccccc5C65c6ccccc6-c6ccccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4c(c3)sc3ccccc34)cc2)c([2H])c1[2H] ; c1ccc(-c2cccc3c2oc2c(N(c4ccc(-c5ccc6c7ccc8ccccc8c7n(-c7ccccc7)c6c5)cc4)c4ccc5c(c4)sc4ccccc45)cccc23)cc1]

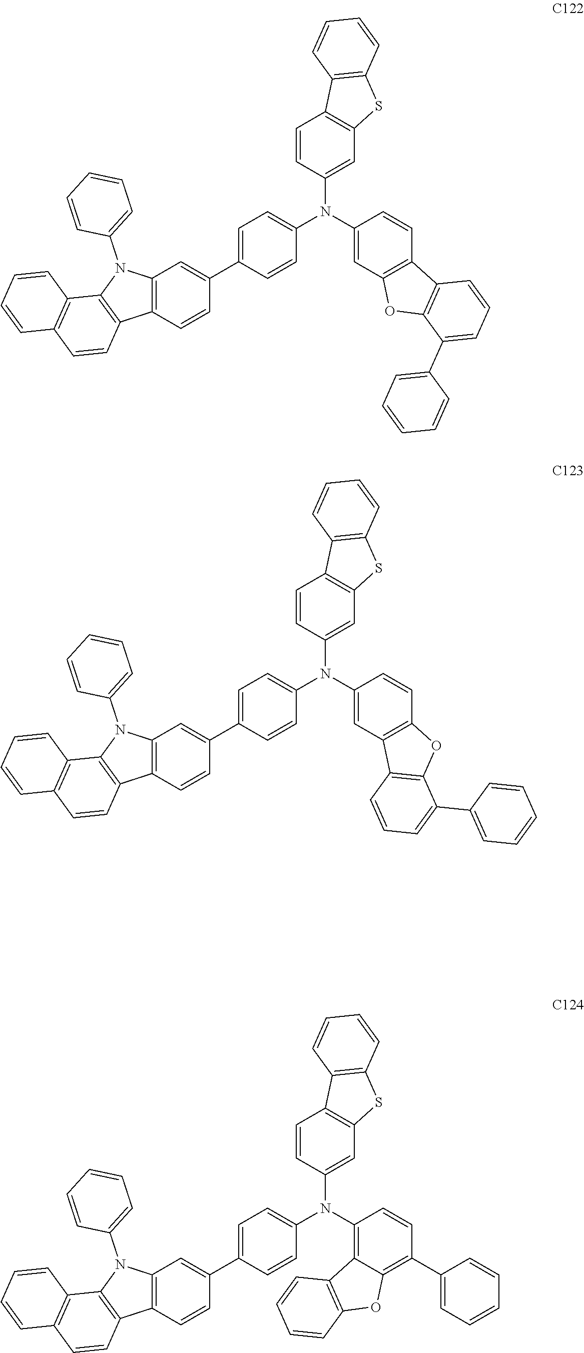 [SMILES: c1ccc(-c2cccc3c2oc2cc(N(c4ccc(-c5ccc6c7ccc8ccccc8c7n(-c7ccccc7)c6c5)cc4)c4ccc5c(c4)sc4ccccc45)ccc23)cc1 ; c1ccc(-c2cccc3c2oc2ccc(N(c4ccc(-c5ccc6c7ccc8ccccc8c7n(-c7ccccc7)c6c5)cc4)c4ccc5c(c4)sc4ccccc45)cc23)cc1 ; c1ccc(-c2ccc(N(c3ccc(-c4ccc5c6ccc7ccccc7c6n(-c6ccccc6)c5c4)cc3)c3ccc4c(c3)sc3ccccc34)c3c2oc2ccccc23)cc1]

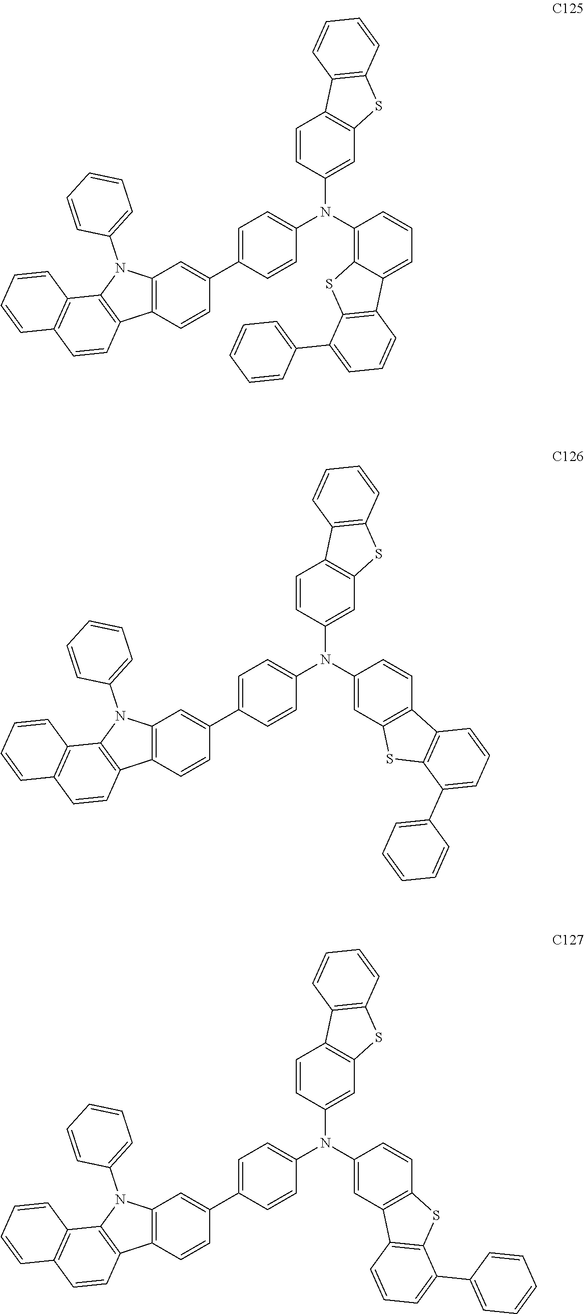 [SMILES: c1ccc(-c2cccc3c2sc2c(N(c4ccc(-c5ccc6c7ccc8ccccc8c7n(-c7ccccc7)c6c5)cc4)c4ccc5c(c4)sc4ccccc45)cccc23)cc1 ; c1ccc(-c2cccc3c2sc2cc(N(c4ccc(-c5ccc6c7ccc8ccccc8c7n(-c7ccccc7)c6c5)cc4)c4ccc5c(c4)sc4ccccc45)ccc23)cc1 ; c1ccc(-c2cccc3c2sc2ccc(N(c4ccc(-c5ccc6c7ccc8ccccc8c7n(-c7ccccc7)c6c5)cc4)c4ccc5c(c4)sc4ccccc45)cc23)cc1]

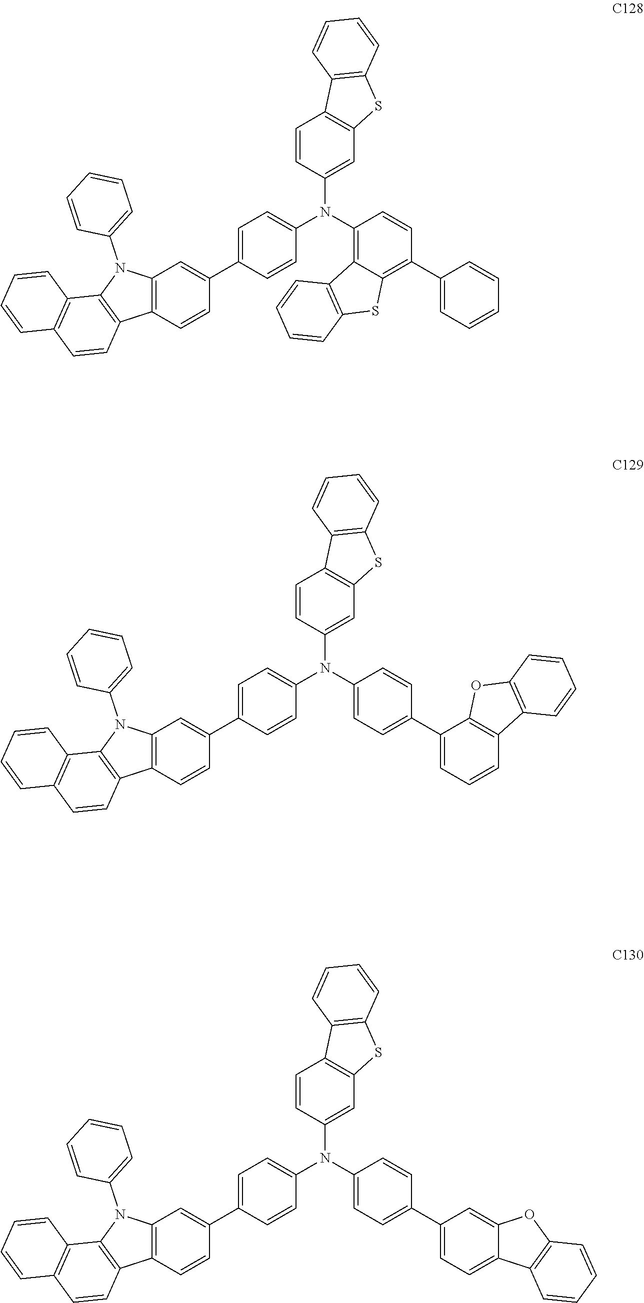 [SMILES: c1ccc(-c2ccc(N(c3ccc(-c4ccc5c6ccc7ccccc7c6n(-c6ccccc6)c5c4)cc3)c3ccc4c(c3)sc3ccccc34)c3c2sc2ccccc23)cc1 ; c1ccc(-n2c3cc(-c4ccc(N(c5ccc(-c6cccc7c6oc6ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5ccc6c(c5)sc5ccccc56)cc4)ccc3c3ccc4ccccc4c32)cc1]

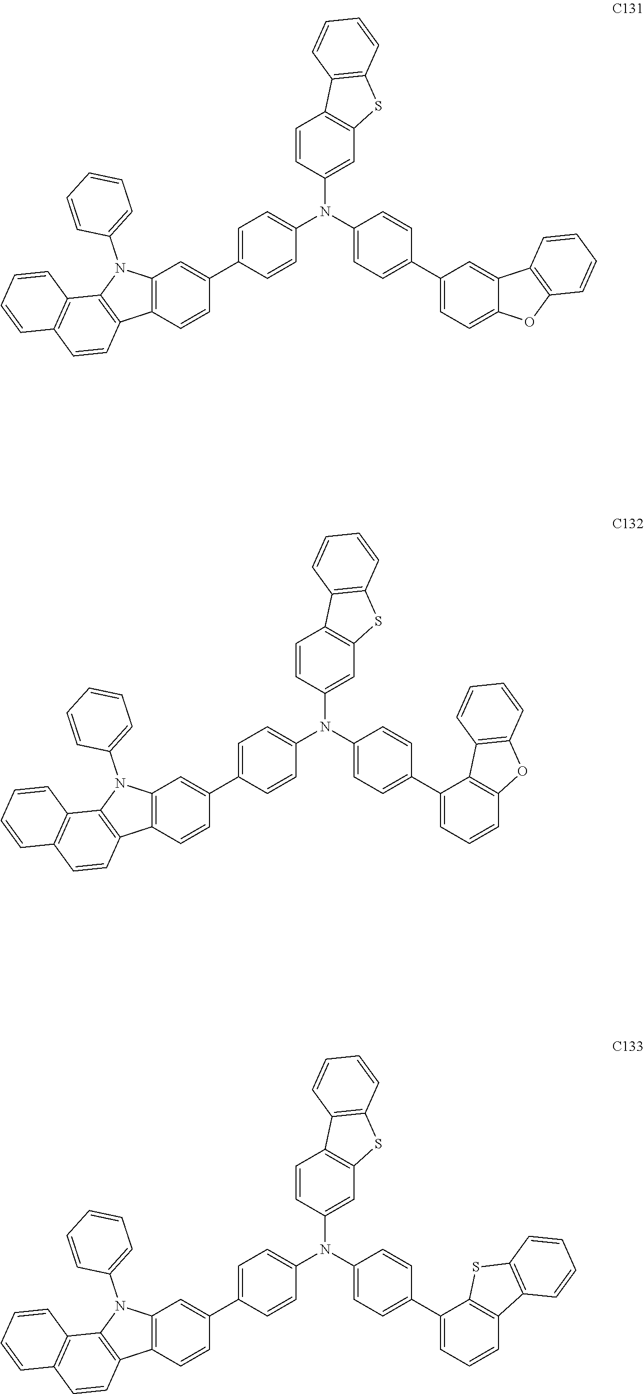 [SMILES: c1ccc(-n2c3cc(-c4ccc(N(c5ccc(-c6ccc7oc8ccccc8c7c6)cc5)c5ccc6c(c5)sc5ccccc56)cc4)ccc3c3ccc4ccccc4c32)cc1 ; c1ccc(-n2c3cc(-c4ccc(N(c5ccc(-c6cccc7oc8ccccc8c67)cc5)c5ccc6c(c5)sc5ccccc56)cc4)ccc3c3ccc4ccccc4c32)cc1 ; c1ccc(-n2c3cc(-c4ccc(N(c5ccc(-c6cccc7c6sc6ccccc67)cc5)c5ccc6c(c5)sc5ccccc56)cc4)ccc3c3ccc4ccccc4c32)cc1]

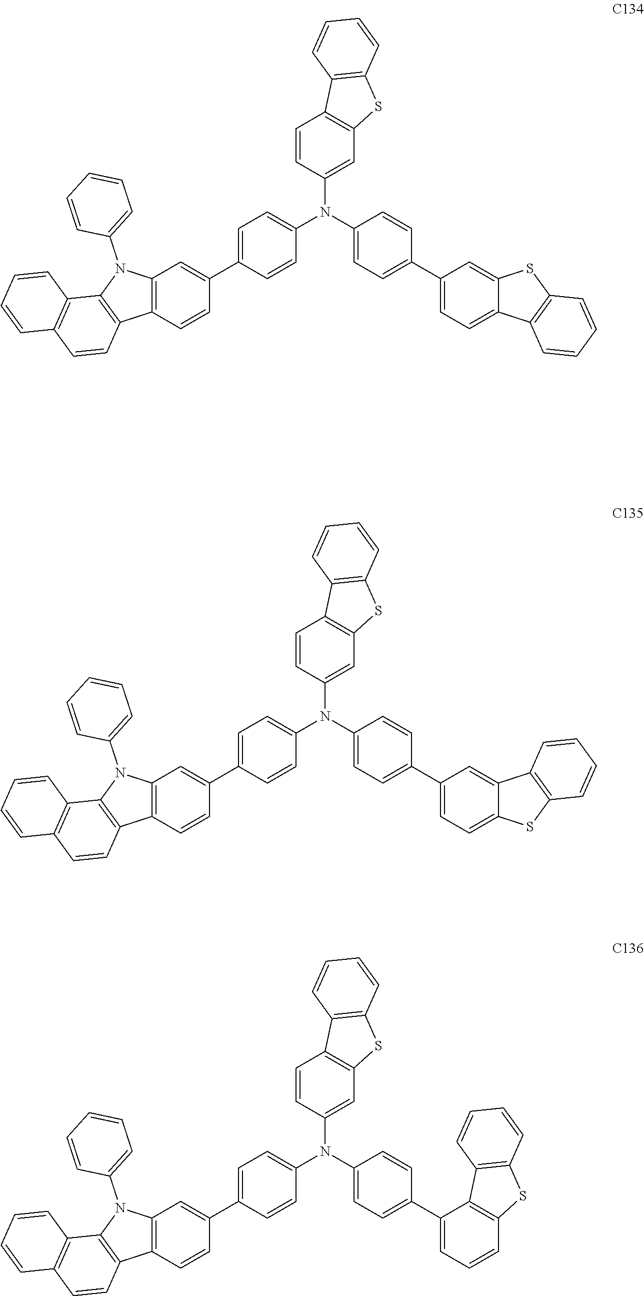 [SMILES: c1ccc(-n2c3cc(-c4ccc(N(c5ccc(-c6ccc7c(c6)sc6ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6ccc7sc8ccccc8c7c6)cc5)c5ccc6c(c5)sc5ccccc56)cc4)ccc3c3ccc4ccccc4c32)cc1 ; c1ccc(-n2c3cc(-c4ccc(N(c5ccc(-c6cccc7sc8ccccc8c67)cc5)c5ccc6c(c5)sc5ccccc56)cc4)ccc3c3ccc4ccccc4c32)cc1]

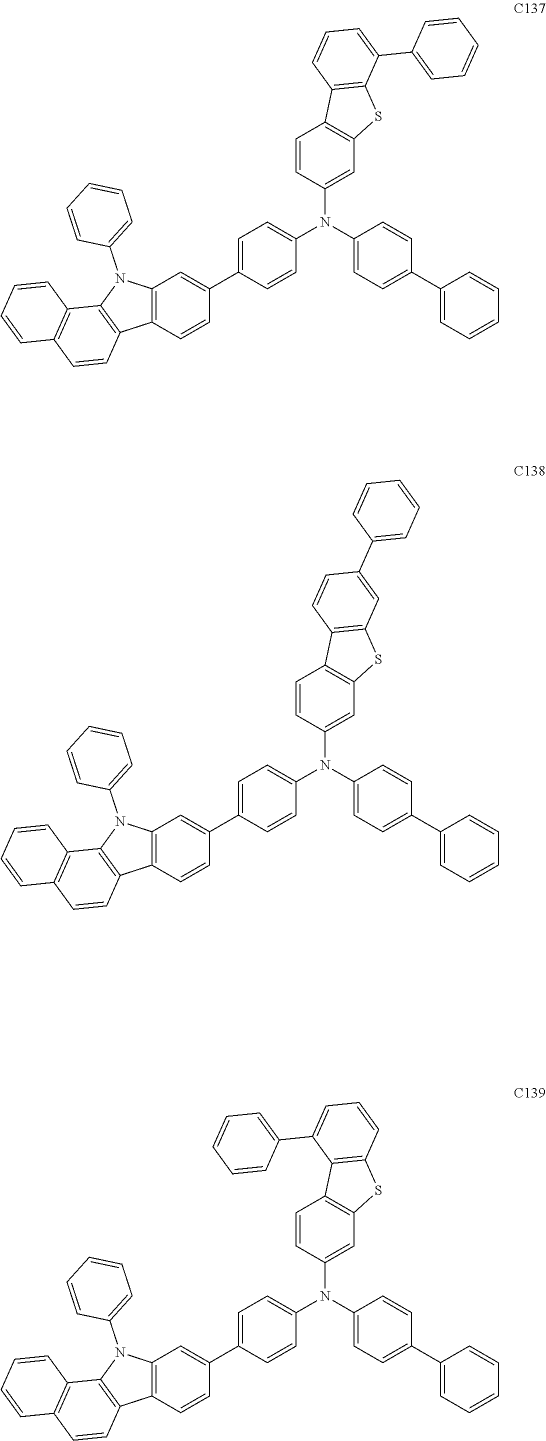 [SMILES: c1ccc(-c2ccc(N(c3ccc(-c4ccc5c6ccc7ccccc7c6n(-c6ccccc6)c5c4)cc3)c3ccc4c(c3)sc3c(-c5ccccc5)cccc34)cc2)cc1 ; c1ccc(-c2ccc(N(c3ccc(-c4ccc5c6ccc7ccccc7c6n(-c6ccccc6)c5c4)cc3)c3ccc4c(c3)sc3cc(-c5ccccc5)ccc34)cc2)cc1 ; c1ccc(-c2ccc(N(c3ccc(-c4ccc5c6ccc7ccccc7c6n(-c6ccccc6)c5c4)cc3)c3ccc4c(c3)sc3cccc(-c5ccccc5)c34)cc2)cc1]

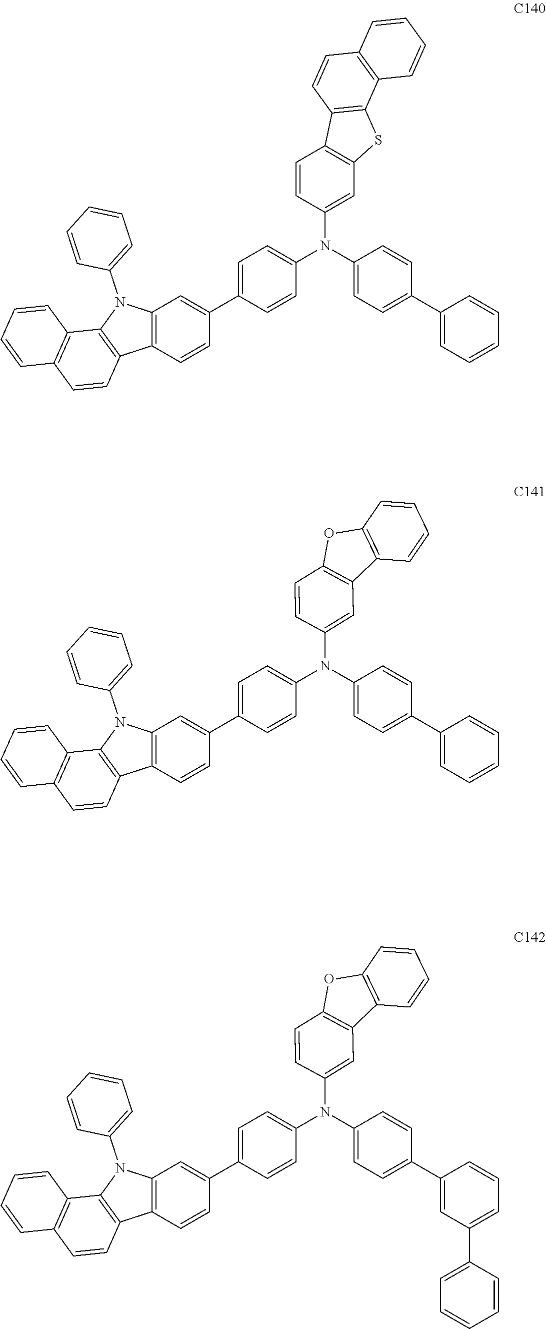 [SMILES: c1ccc(-c2ccc(N(c3ccc(-c4ccc5c6ccc7ccccc7c6n(-c6ccccc6)c5c4)cc3)c3ccc4c(c3)sc3c5ccccc5ccc43)cc2)cc1 ; c1ccc(-c2ccc(N(c3ccc(-c4ccc5c6ccc7ccccc7c6n(-c6ccccc6)c5c4)cc3)c3ccc4oc5ccccc5c4c3)cc2)cc1 ; c1ccc(-c2cccc(-c3ccc(N(c4ccc(-c5ccc6c7ccc8ccccc8c7n(-c7ccccc7)c6c5)cc4)c4ccc5oc6ccccc6c5c4)cc3)c2)cc1]

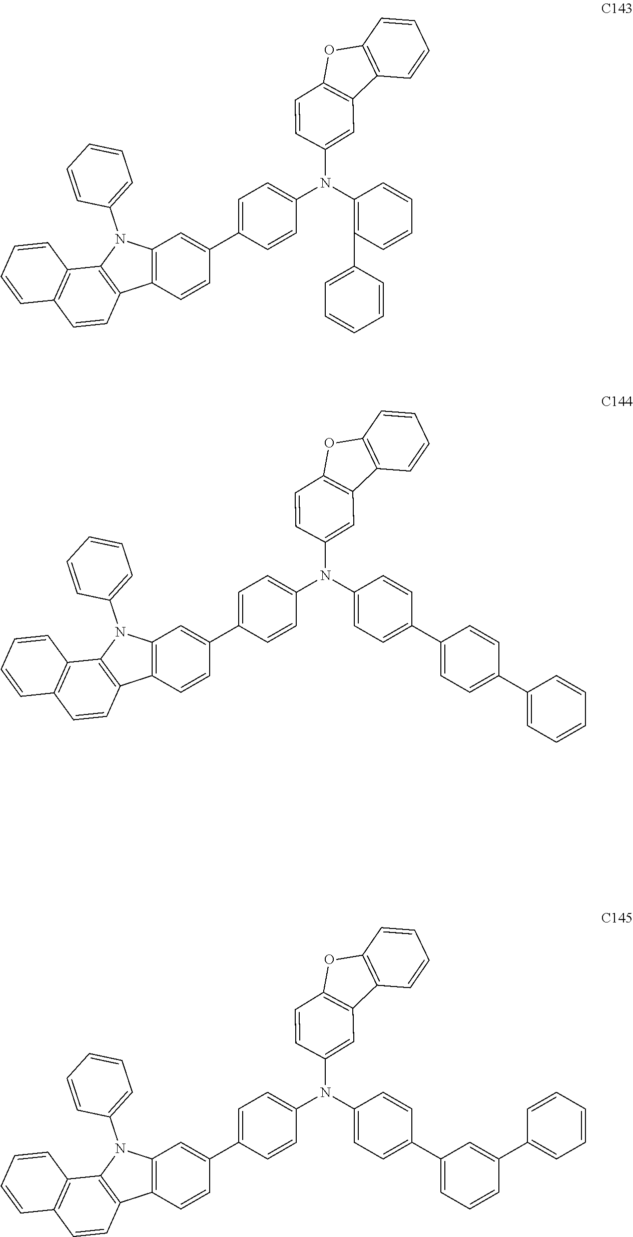 [SMILES: c1ccc(-c2ccccc2N(c2ccc(-c3ccc4c5ccc6ccccc6c5n(-c5ccccc5)c4c3)cc2)c2ccc3oc4ccccc4c3c2)cc1 ; c1ccc(-c2ccc(-c3ccc(N(c4ccc(-c5ccc6c7ccc8ccccc8c7n(-c7ccccc7)c6c5)cc4)c4ccc5oc6ccccc6c5c4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5oc6ccccc6c5c4)cc3)c2)cc1]

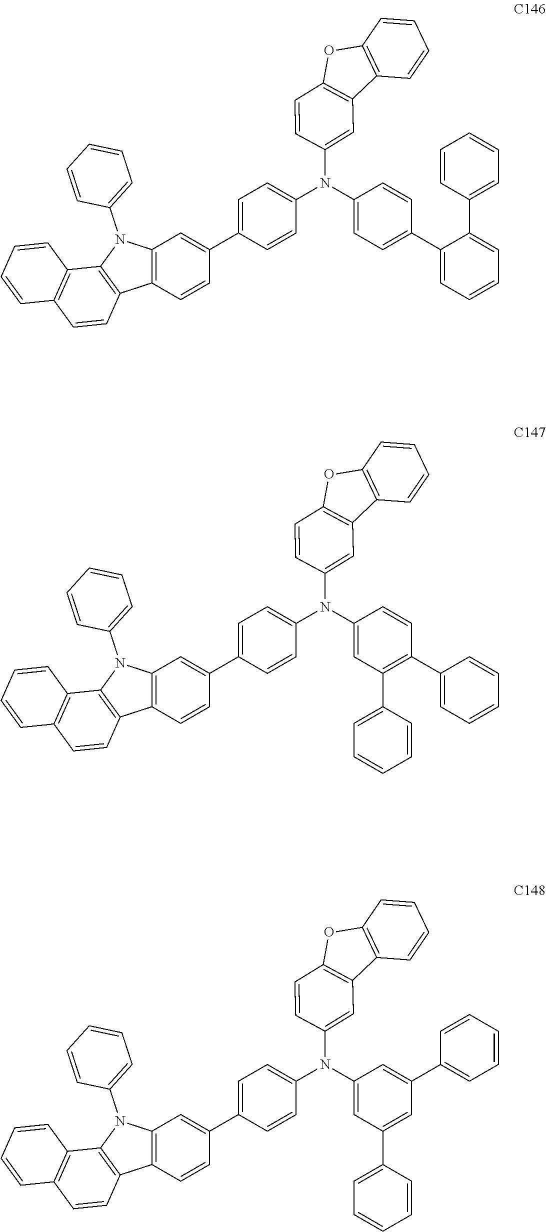 [SMILES: c1ccc(-c2ccccc2-c2ccc(N(c3ccc(-c4ccc5c6ccc7ccccc7c6n(-c6ccccc6)c5c4)cc3)c3ccc4oc5ccccc5c4c3)cc2)cc1 ; c1ccc(-c2ccc(N(c3ccc(-c4ccc5c6ccc7ccccc7c6n(-c6ccccc6)c5c4)cc3)c3ccc4oc5ccccc5c4c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4oc5ccccc5c4c3)c2)cc1]

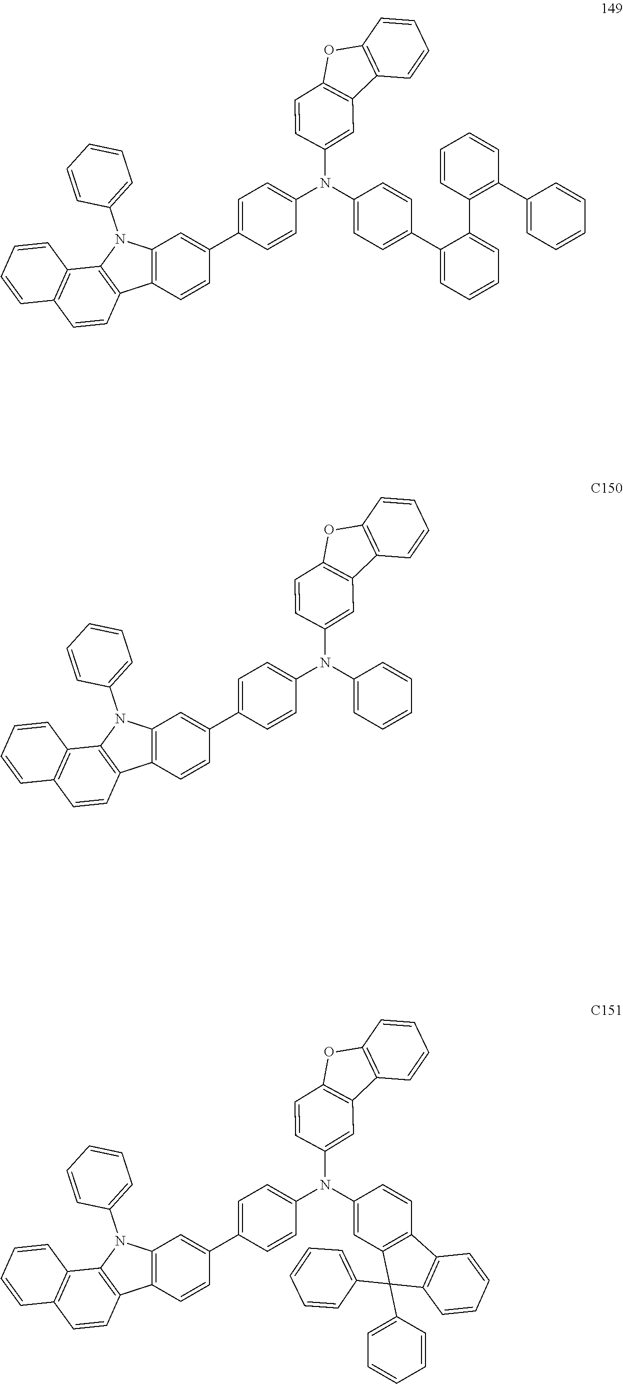 [SMILES: c1ccc(-c2ccccc2-c2ccccc2-c2ccc(N(c3ccc(-c4ccc5c6ccc7ccccc7c6n(-c6ccccc6)c5c4)cc3)c3ccc4oc5ccccc5c4c3)cc2)cc1 ; c1ccc(N(c2ccc(-c3ccc4c5ccc6ccccc6c5n(-c5ccccc5)c4c3)cc2)c2ccc3oc4ccccc4c3c2)cc1 ; c1ccc(-n2c3cc(-c4ccc(N(c5ccc6c(c5)C(c5ccccc5)(c5ccccc5)c5ccccc5-6)c5ccc6oc7ccccc7c6c5)cc4)ccc3c3ccc4ccccc4c32)cc1]

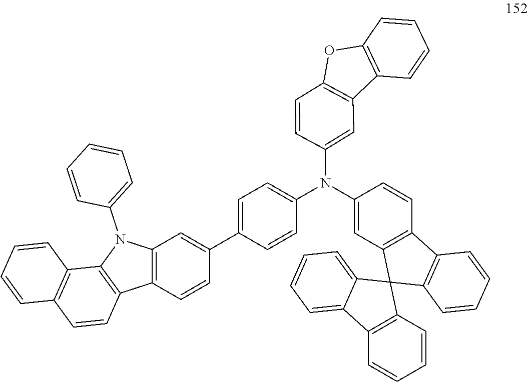 [SMILES: c1ccc(-n2c3cc(-c4ccc(N(c5ccc6c(c5)C5(c7ccccc7-c7ccccc75)c5ccccc5-6)c5ccc6oc7ccccc7c6c5)cc4)ccc3c3ccc4ccccc4c32)cc1]

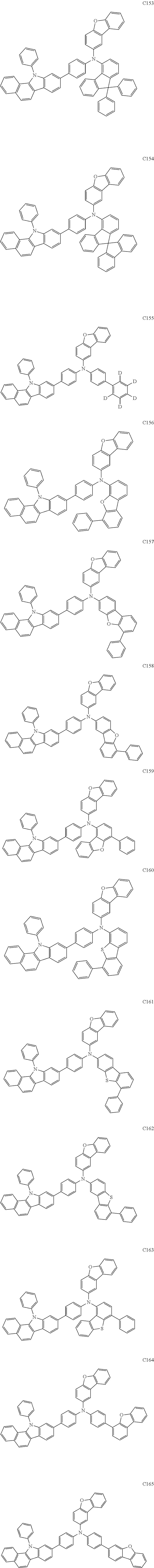

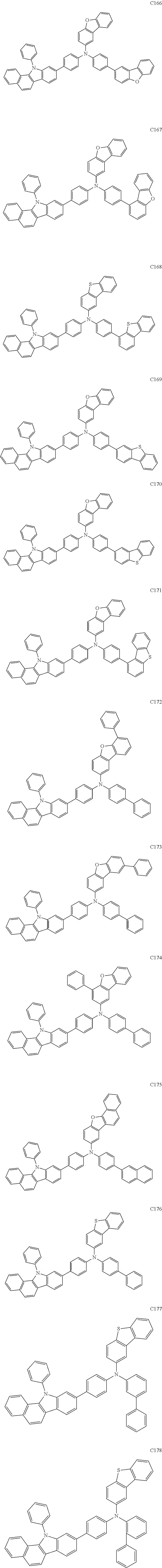

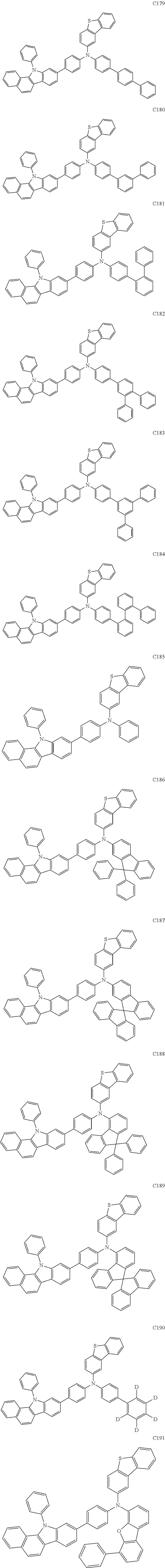

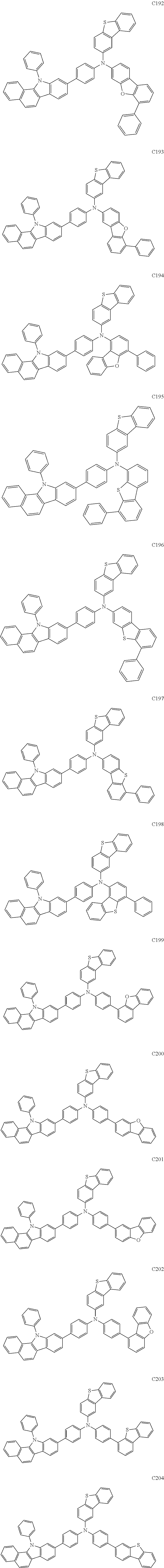

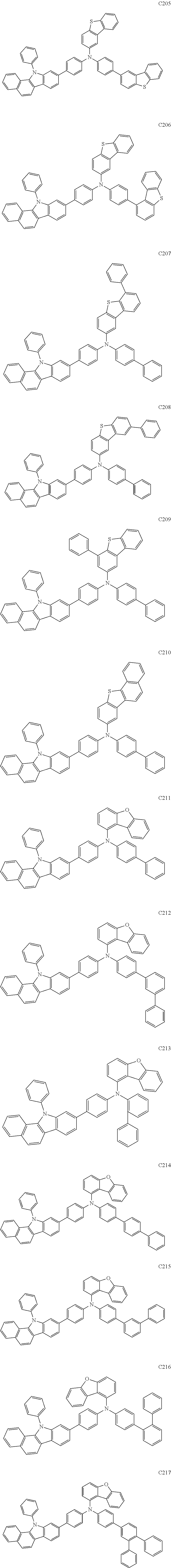

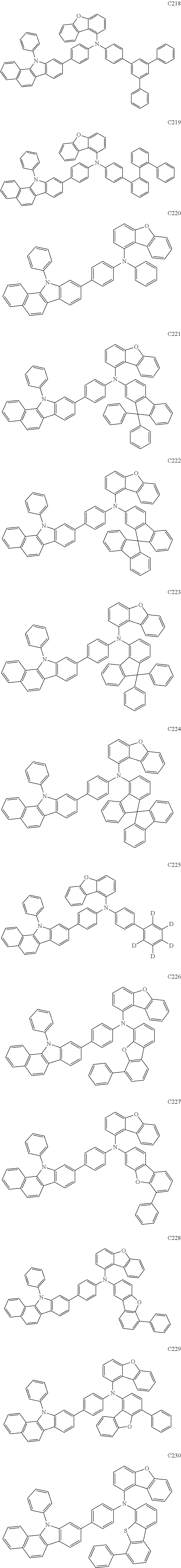

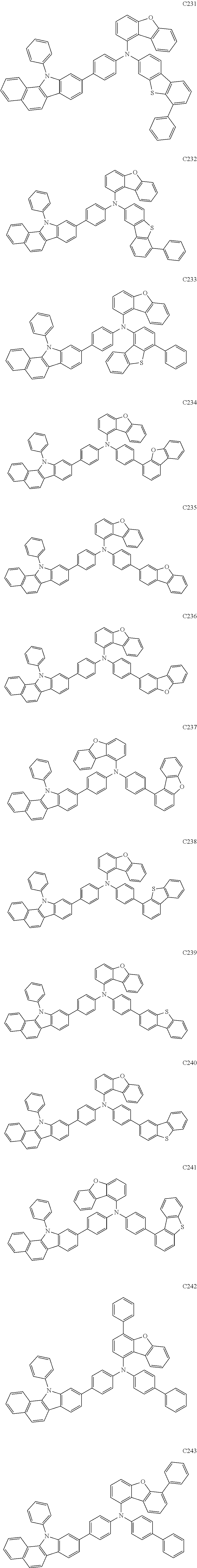

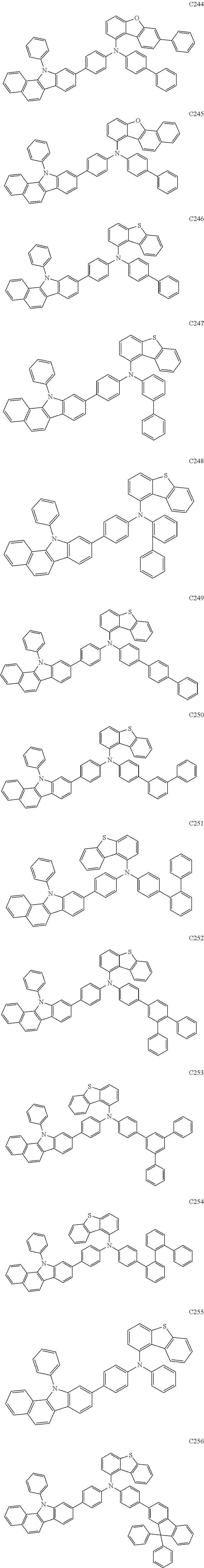

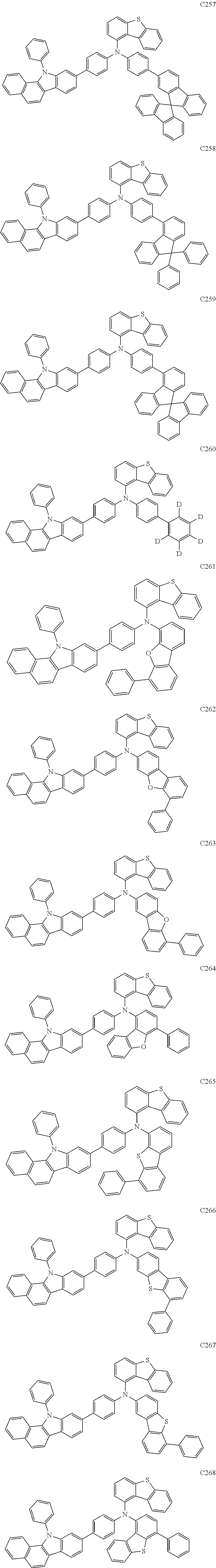

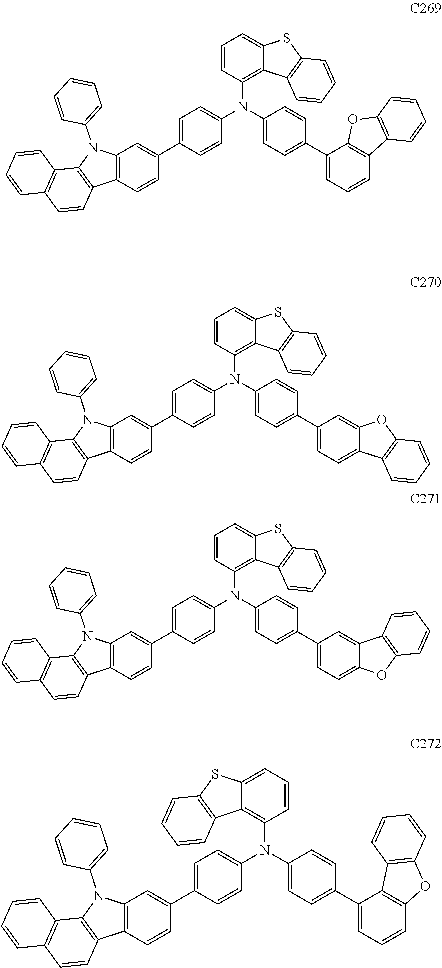 [SMILES: c1ccc(-n2c3cc(-c4ccc(N(c5ccc(-c6cccc7c6oc6ccccc67)cc5)c5cccc6sc7ccccc7c56)cc4)ccc3c3ccc4ccccc4c32)cc1 ; c1ccc(-n2c3cc(-c4ccc(N(c5ccc(-c6ccc7c(c6)oc6ccccc67)cc5)c5cccc6sc7ccccc7c56)cc4)ccc3c3ccc4ccccc4c32)cc1 ; c1ccc(-n2c3cc(-c4ccc(N(c5ccc(-c6ccc7oc8ccccc8c7c6)cc5)c5cccc6sc7ccccc7c56)cc4)ccc3c3ccc4ccccc4c32)cc1 ; c1ccc(-n2c3cc(-c4ccc(N(c5ccc(-c6cccc7oc8ccccc8c67)cc5)c5cccc6sc7ccccc7c56)cc4)ccc3c3ccc4ccccc4c32)cc1]

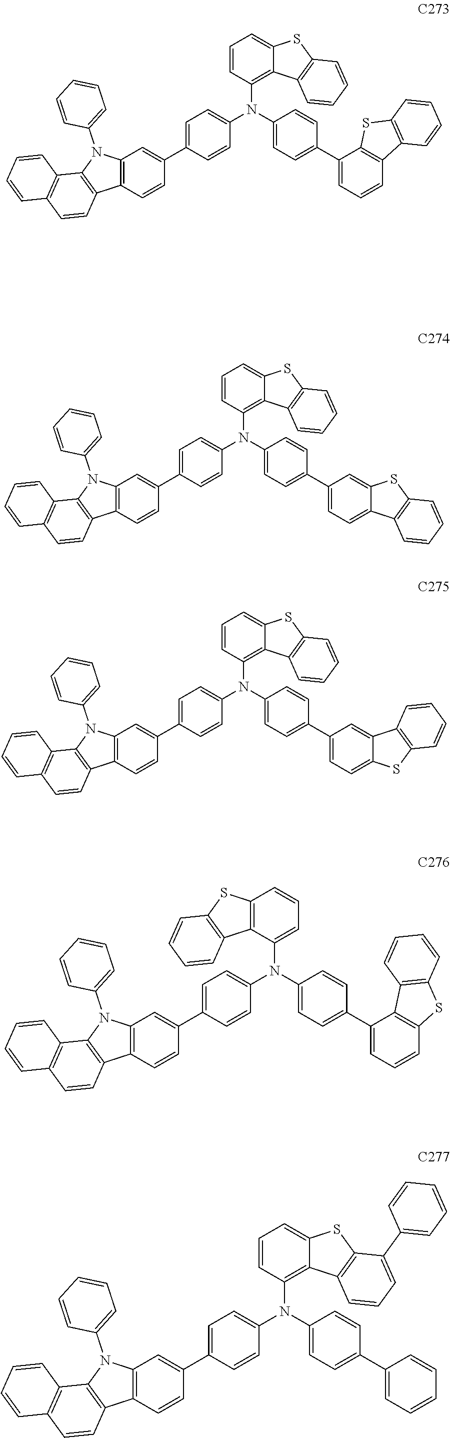

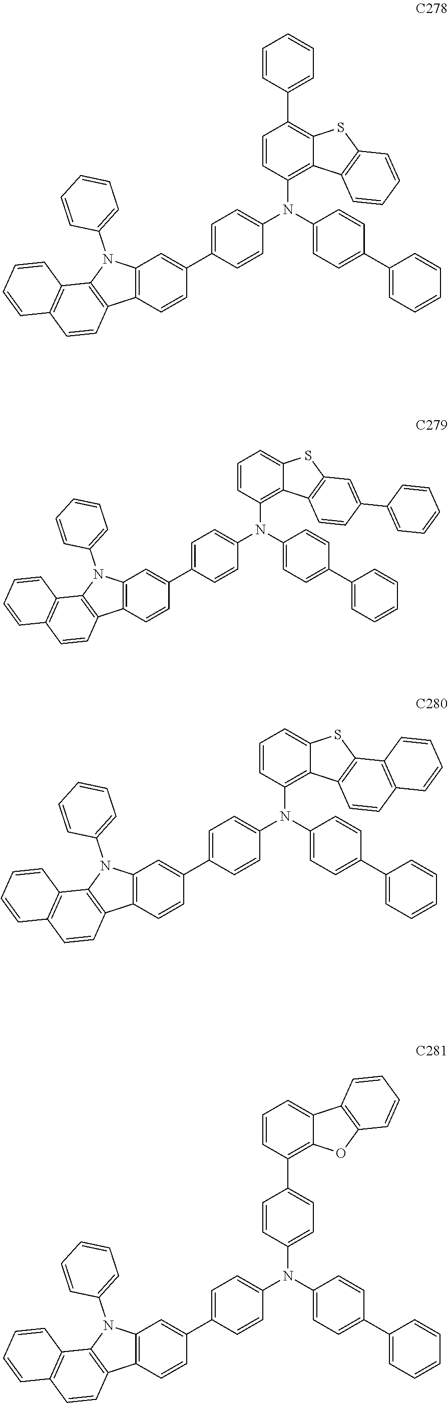 [SMILES: c1ccc(-c2ccc(N(c3ccc(-c4ccc5c6ccc7ccccc7c6n(-c6ccccc6)c5c4)cc3)c3ccc(-c4ccccc4)c4sc5ccccc5c34)cc2)cc1 ; c1ccc(-c2ccc(N(c3ccc(-c4ccc5c6ccc7ccccc7c6n(-c6ccccc6)c5c4)cc3)c3cccc4sc5cc(-c6ccccc6)ccc5c34)cc2)cc1 ; c1ccc(-c2ccc(N(c3ccc(-c4ccc5c6ccc7ccccc7c6n(-c6ccccc6)c5c4)cc3)c3cccc4sc5c6ccccc6ccc5c34)cc2)cc1 ; c1ccc(-c2ccc(N(c3ccc(-c4ccc5c6ccc7ccccc7c6n(-c6ccccc6)c5c4)cc3)c3ccc(-c4cccc5c4oc4ccccc45)cc3)cc2)cc1]

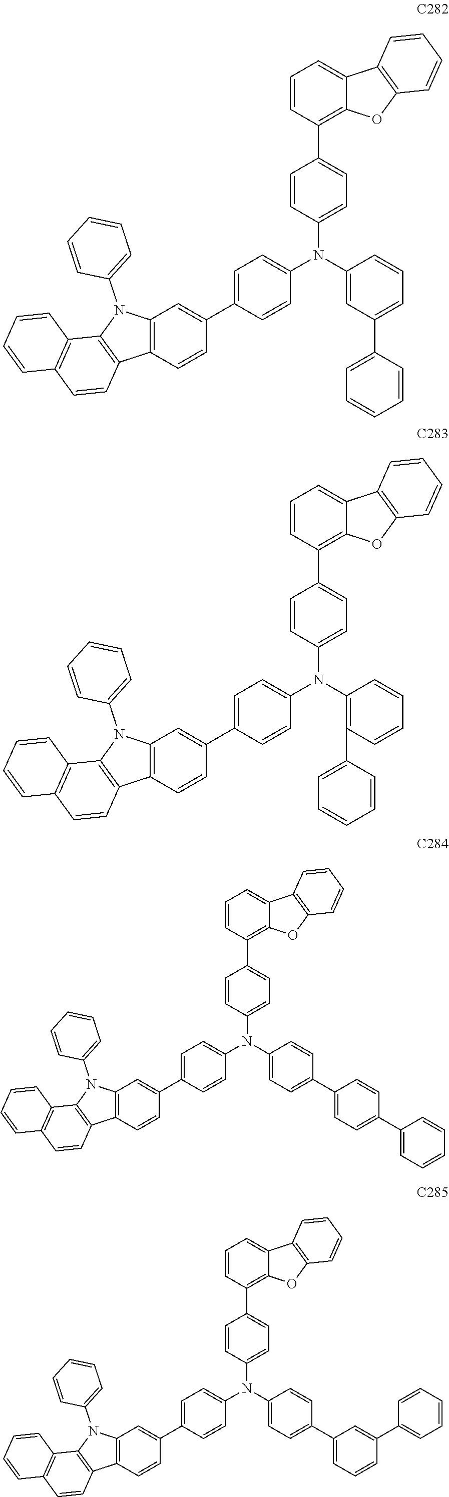 [SMILES: c1ccc(-c2cccc(N(c3ccc(-c4ccc5c6ccc7ccccc7c6n(-c6ccccc6)c5c4)cc3)c3ccc(-c4cccc5c4oc4ccccc45)cc3)c2)cc1 ; c1ccc(-c2ccccc2N(c2ccc(-c3ccc4c5ccc6ccccc6c5n(-c5ccccc5)c4c3)cc2)c2ccc(-c3cccc4c3oc3ccccc34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6c5oc5ccccc56)cc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc(-c5cccc6c5oc5ccccc56)cc4)cc3)c2)cc1]

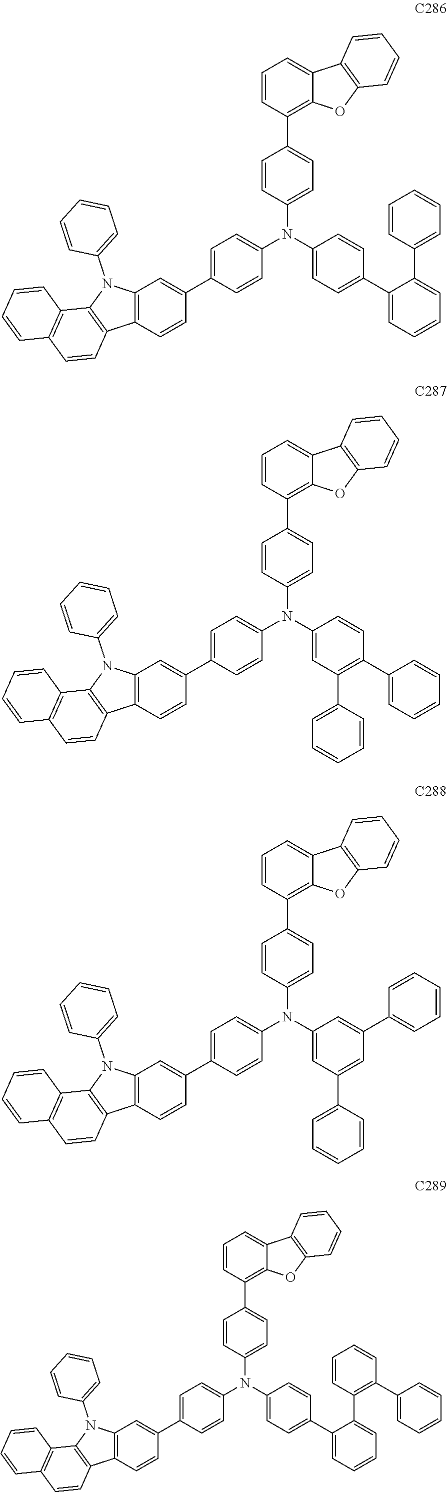 [SMILES: c1ccc(-c2ccccc2-c2ccc(N(c3ccc(-c4ccc5c6ccc7ccccc7c6n(-c6ccccc6)c5c4)cc3)c3ccc(-c4cccc5c4oc4ccccc45)cc3)cc2)cc1 ; c1ccc(-c2ccc(N(c3ccc(-c4ccc5c6ccc7ccccc7c6n(-c6ccccc6)c5c4)cc3)c3ccc(-c4cccc5c4oc4ccccc45)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cccc5c4oc4ccccc45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5c4oc4ccccc45)cc3)cc2)cc1]

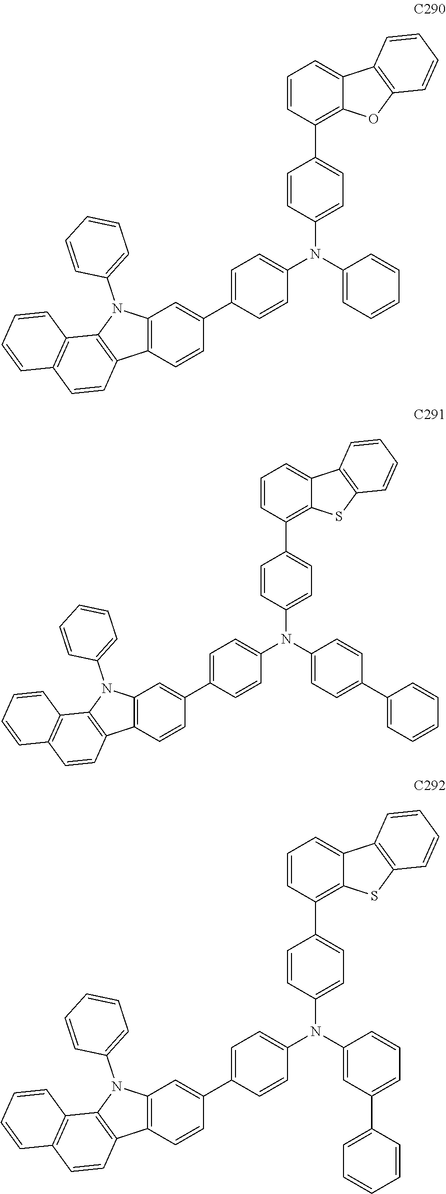 [SMILES: c1ccc(N(c2ccc(-c3ccc4c5ccc6ccccc6c5n(-c5ccccc5)c4c3)cc2)c2ccc(-c3cccc4c3oc3ccccc34)cc2)cc1 ; c1ccc(-c2ccc(N(c3ccc(-c4ccc5c6ccc7ccccc7c6n(-c6ccccc6)c5c4)cc3)c3ccc(-c4cccc5c4sc4ccccc45)cc3)cc2)cc1 ; c1ccc(-c2cccc(N(c3ccc(-c4ccc5c6ccc7ccccc7c6n(-c6ccccc6)c5c4)cc3)c3ccc(-c4cccc5c4sc4ccccc45)cc3)c2)cc1]

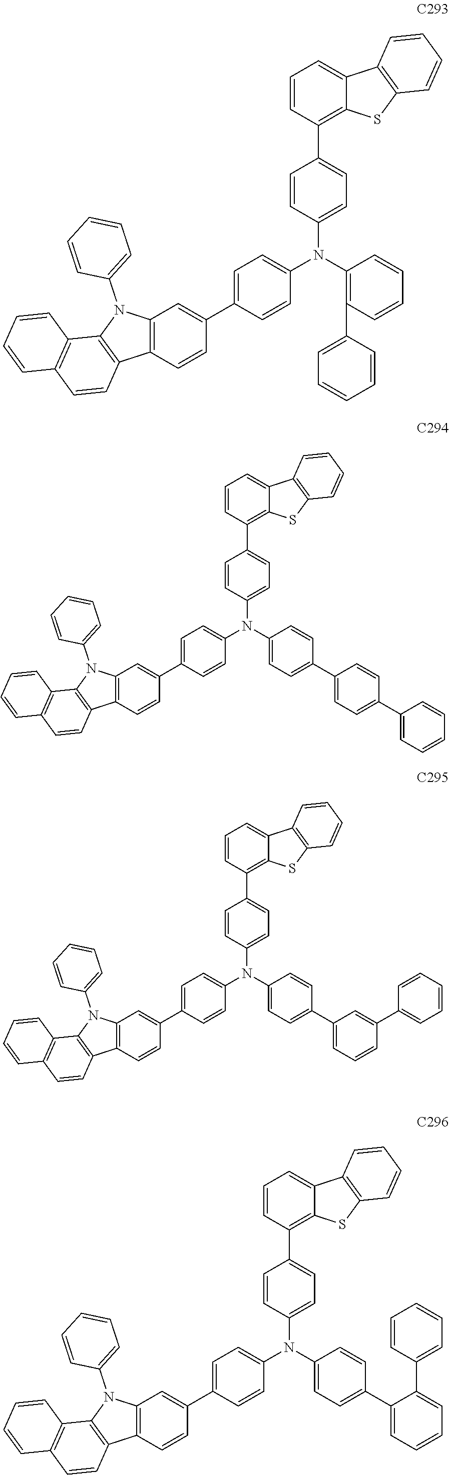 [SMILES: c1ccc(-c2ccccc2N(c2ccc(-c3ccc4c5ccc6ccccc6c5n(-c5ccccc5)c4c3)cc2)c2ccc(-c3cccc4c3sc3ccccc34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6c5sc5ccccc56)cc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc(-c5cccc6c5sc5ccccc56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5c4sc4ccccc45)cc3)cc2)cc1]

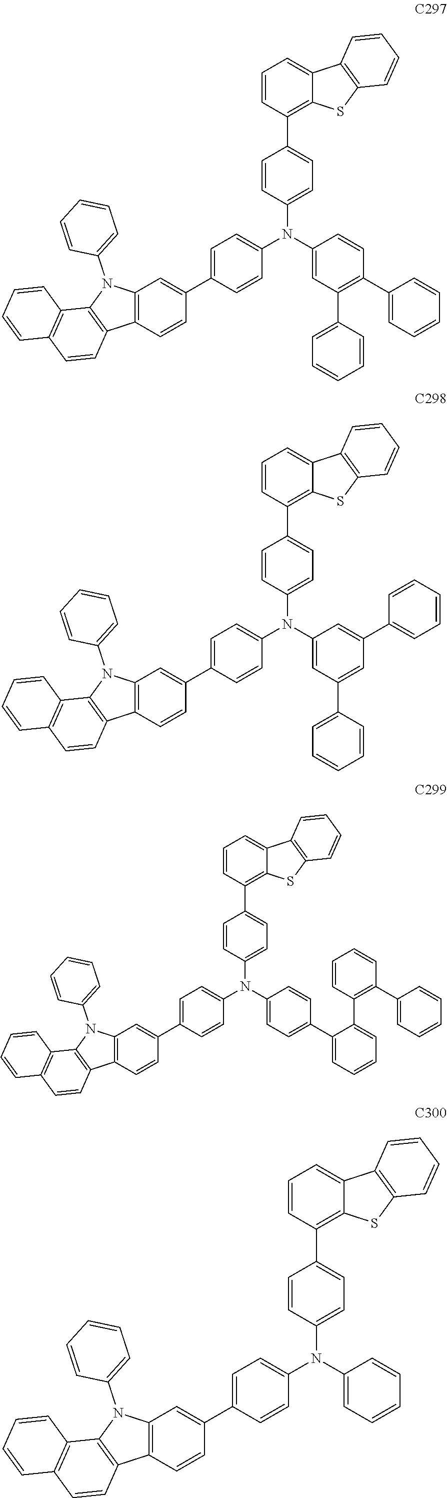 [SMILES: c1ccc(-c2ccc(N(c3ccc(-c4ccc5c6ccc7ccccc7c6n(-c6ccccc6)c5c4)cc3)c3ccc(-c4cccc5c4sc4ccccc45)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cccc5c4sc4ccccc45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5c4sc4ccccc45)cc3)cc2)cc1 ; c1ccc(N(c2ccc(-c3ccc4c5ccc6ccccc6c5n(-c5ccccc5)c4c3)cc2)c2ccc(-c3cccc4c3sc3ccccc34)cc2)cc1]

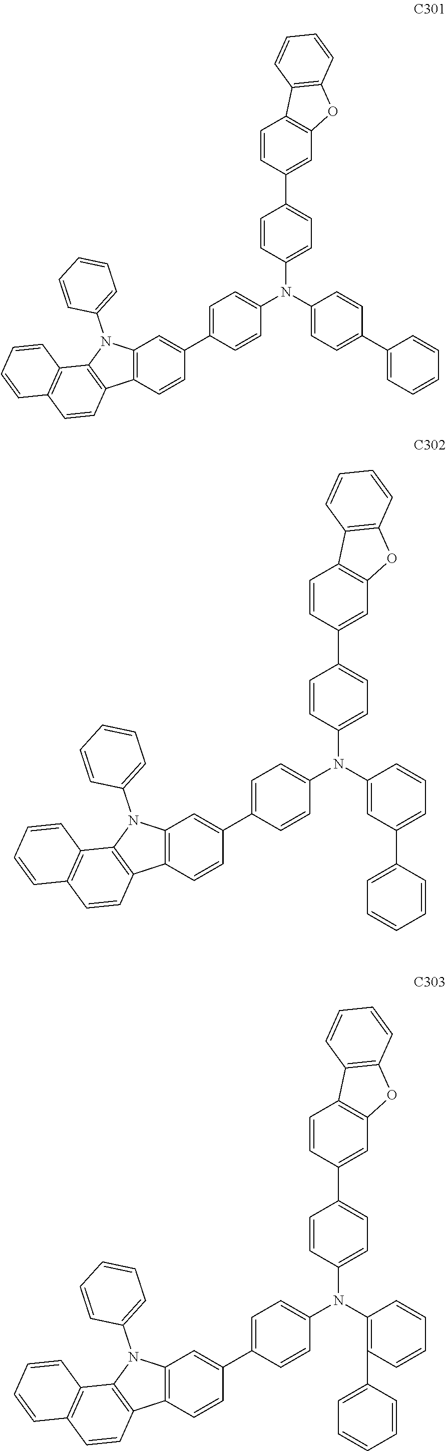 [SMILES: c1ccc(-c2ccc(N(c3ccc(-c4ccc5c(c4)oc4ccccc45)cc3)c3ccc(-c4ccc5c6ccc7ccccc7c6n(-c6ccccc6)c5c4)cc3)cc2)cc1 ; c1ccc(-c2cccc(N(c3ccc(-c4ccc5c(c4)oc4ccccc45)cc3)c3ccc(-c4ccc5c6ccc7ccccc7c6n(-c6ccccc6)c5c4)cc3)c2)cc1 ; c1ccc(-c2ccccc2N(c2ccc(-c3ccc4c(c3)oc3ccccc34)cc2)c2ccc(-c3ccc4c5ccc6ccccc6c5n(-c5ccccc5)c4c3)cc2)cc1]

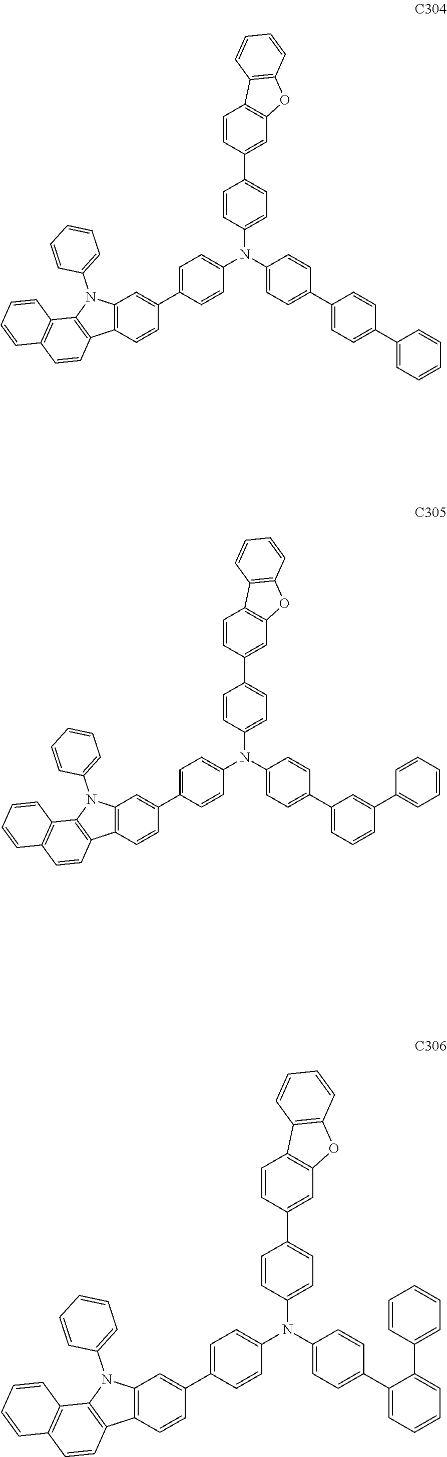 [SMILES: c1ccc(-c2ccc(-c3ccc(N(c4ccc(-c5ccc6c(c5)oc5ccccc56)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6c(c5)oc5ccccc56)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5c(c4)oc4ccccc45)cc3)c3ccc(-c4ccc5c6ccc7ccccc7c6n(-c6ccccc6)c5c4)cc3)cc2)cc1]

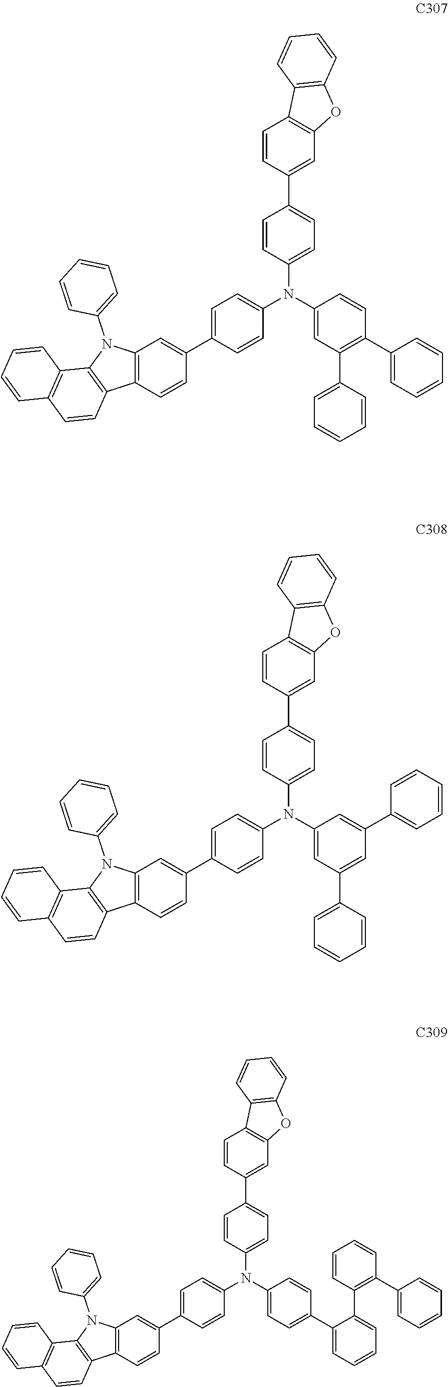 [SMILES: c1ccc(-c2ccc(N(c3ccc(-c4ccc5c(c4)oc4ccccc45)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5c(c4)oc4ccccc45)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5c(c4)oc4ccccc45)cc3)c3ccc(-c4ccc5c6ccc7ccccc7c6n(-c6ccccc6)c5c4)cc3)cc2)cc1]

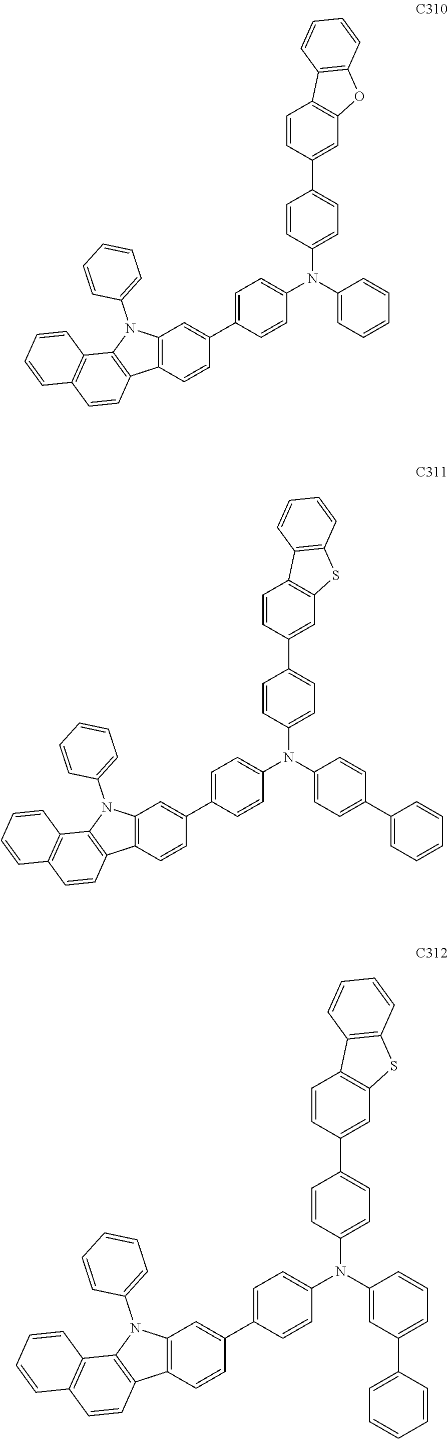 [SMILES: c1ccc(N(c2ccc(-c3ccc4c(c3)oc3ccccc34)cc2)c2ccc(-c3ccc4c5ccc6ccccc6c5n(-c5ccccc5)c4c3)cc2)cc1 ; c1ccc(-c2ccc(N(c3ccc(-c4ccc5c(c4)sc4ccccc45)cc3)c3ccc(-c4ccc5c6ccc7ccccc7c6n(-c6ccccc6)c5c4)cc3)cc2)cc1 ; c1ccc(-c2cccc(N(c3ccc(-c4ccc5c(c4)sc4ccccc45)cc3)c3ccc(-c4ccc5c6ccc7ccccc7c6n(-c6ccccc6)c5c4)cc3)c2)cc1]

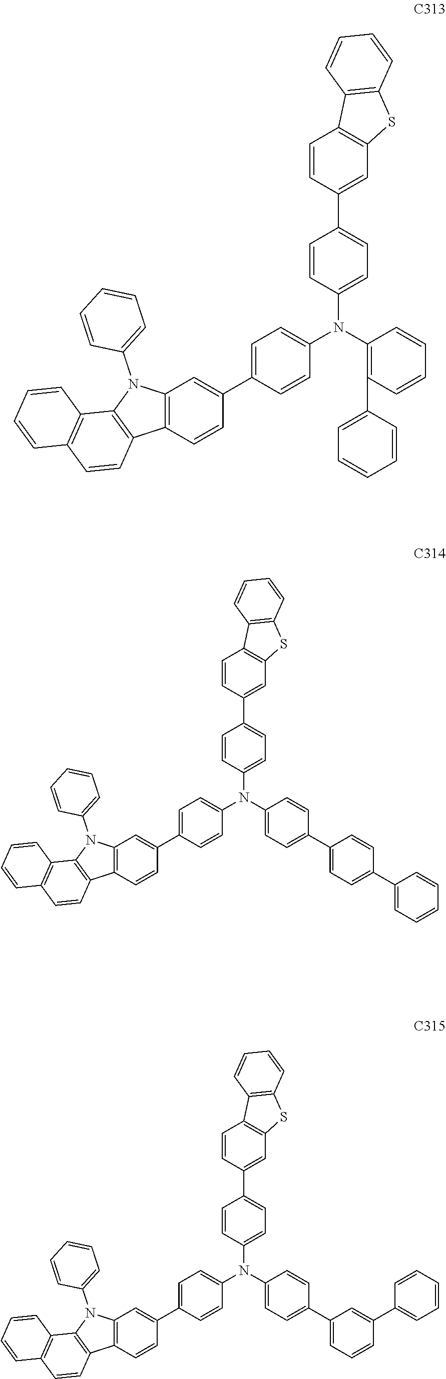 [SMILES: c1ccc(-c2ccccc2N(c2ccc(-c3ccc4c(c3)sc3ccccc34)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6c(c5)sc5ccccc56)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6c(c5)sc5ccccc56)cc4)c4ccc(-c5ccc6c7ccc8ccccc8c7n(-c7ccccc7)c6c5)cc4)cc3)c2)cc1]

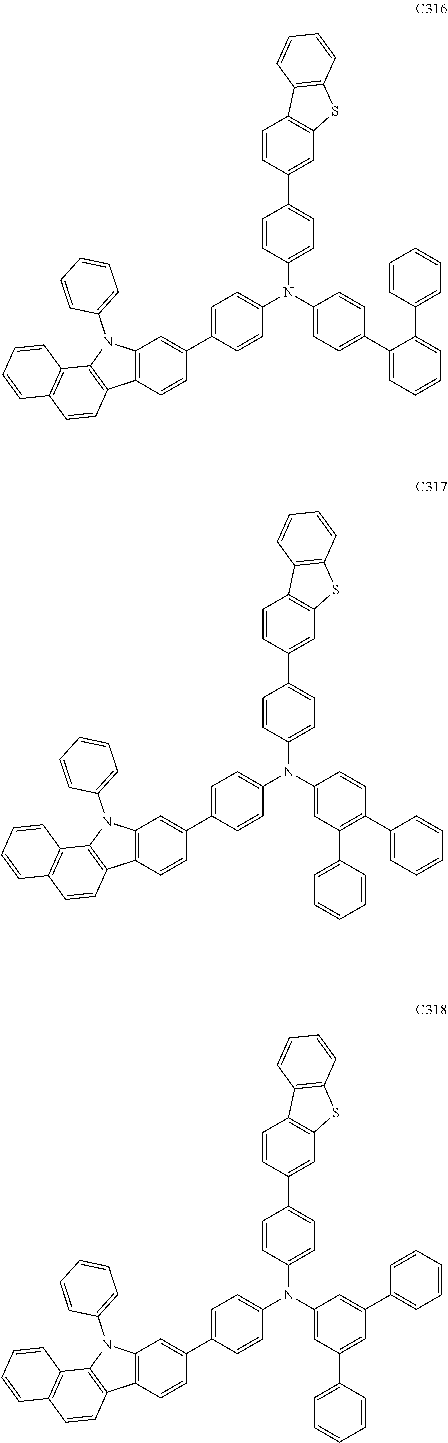 [SMILES: c1ccc(-c2ccccc2-c2ccc(N(c3ccc(-c4ccc5c(c4)sc4ccccc45)cc3)c3ccc(-c4ccc5c6ccc7ccccc7c6n(-c6ccccc6)c5c4)cc3)cc2)cc1 ; c1ccc(-c2ccc(N(c3ccc(-c4ccc5c(c4)sc4ccccc45)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5c(c4)sc4ccccc45)cc3)c3ccc(-c4ccc5c6ccc7ccccc7c6n(-c6ccccc6)c5c4)cc3)c2)cc1]

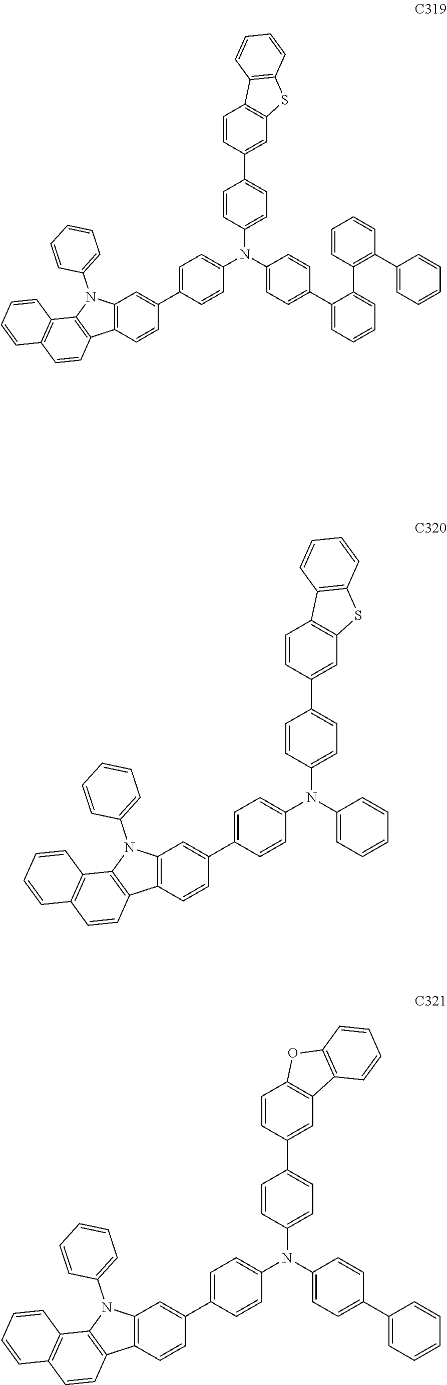 [SMILES: c1ccc(-c2ccccc2-c2ccccc2-c2ccc(N(c3ccc(-c4ccc5c(c4)sc4ccccc45)cc3)c3ccc(-c4ccc5c6ccc7ccccc7c6n(-c6ccccc6)c5c4)cc3)cc2)cc1 ; c1ccc(N(c2ccc(-c3ccc4c(c3)sc3ccccc34)cc2)c2ccc(-c3ccc4c5ccc6ccccc6c5n(-c5ccccc5)c4c3)cc2)cc1 ; c1ccc(-c2ccc(N(c3ccc(-c4ccc5oc6ccccc6c5c4)cc3)c3ccc(-c4ccc5c6ccc7ccccc7c6n(-c6ccccc6)c5c4)cc3)cc2)cc1]

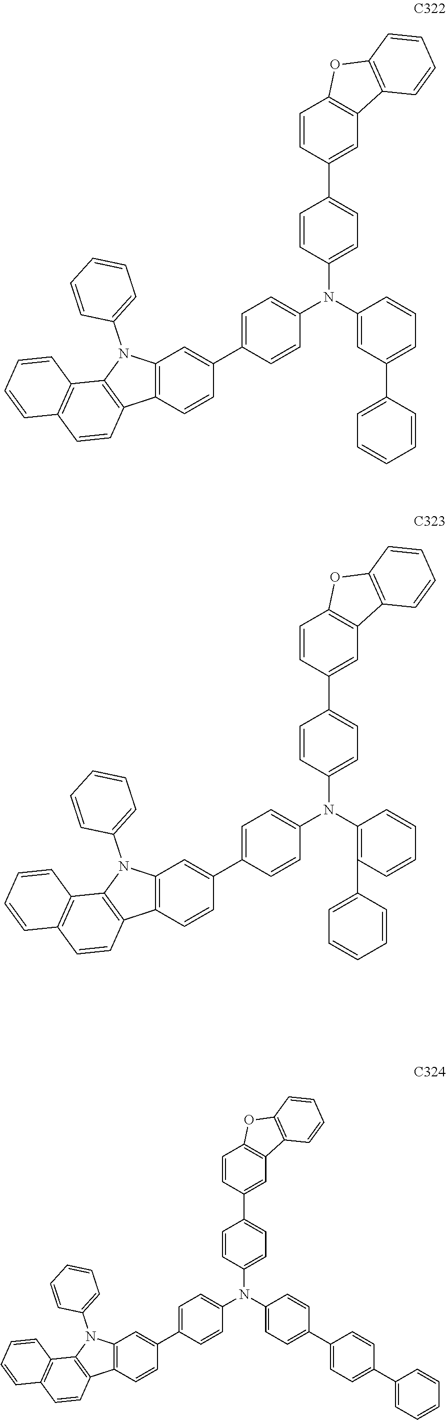 [SMILES: c1ccc(-c2cccc(N(c3ccc(-c4ccc5oc6ccccc6c5c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4oc5ccccc5c4c3)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6oc7ccccc7c6c5)cc4)c4ccc(-c5ccc6c7ccc8ccccc8c7n(-c7ccccc7)c6c5)cc4)cc3)cc2)cc1]

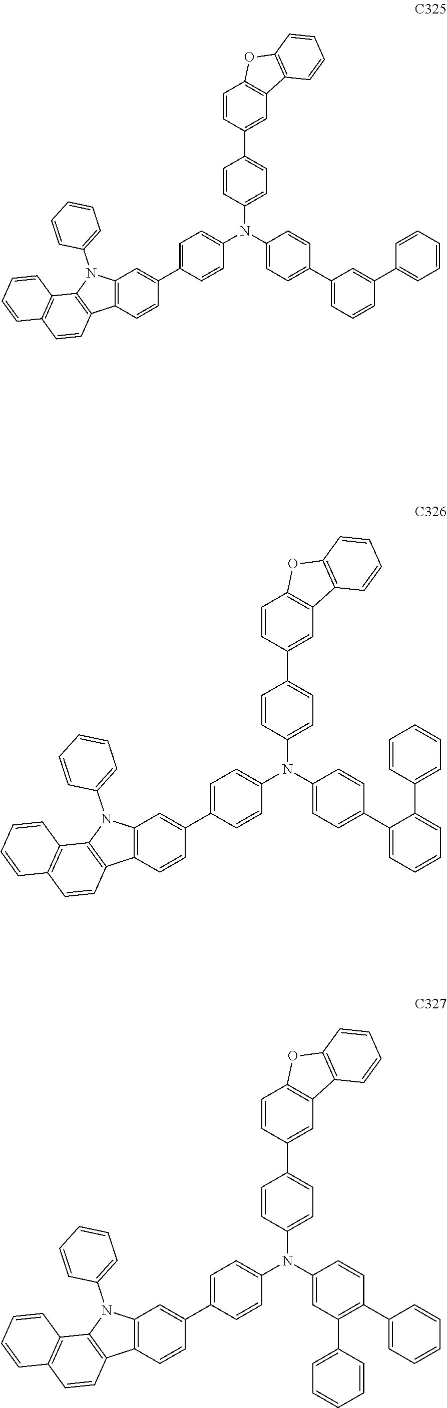 [SMILES: c1ccc(-c2cccc(-c3ccc(N(c4ccc(-c5ccc6oc7ccccc7c6c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5oc6ccccc6c5c4)cc3)c3ccc(-c4ccc5c6ccc7ccccc7c6n(-c6ccccc6)c5c4)cc3)cc2)cc1 ; c1ccc(-c2ccc(N(c3ccc(-c4ccc5oc6ccccc6c5c4)cc3)c3ccc(-c4ccc5c6ccc7ccccc7c6n(-c6ccccc6)c5c4)cc3)cc2-c2ccccc2)cc1]

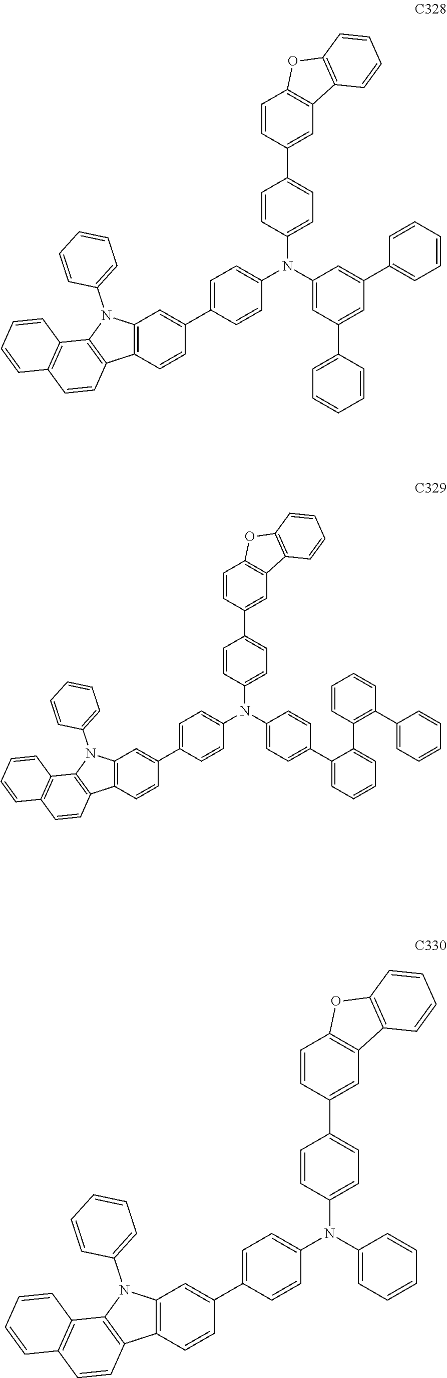 [SMILES: c1ccc(-c2cc(-c3ccccc3)cc(N(c3ccc(-c4ccc5oc6ccccc6c5c4)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5oc6ccccc6c5c4)cc3)c3ccc(-c4ccc5c6ccc7ccccc7c6n(-c6ccccc6)c5c4)cc3)cc2)cc1 ; c1ccc(N(c2ccc(-c3ccc4oc5ccccc5c4c3)cc2)c2ccc(-c3ccc4c5ccc6ccccc6c5n(-c5ccccc5)c4c3)cc2)cc1]

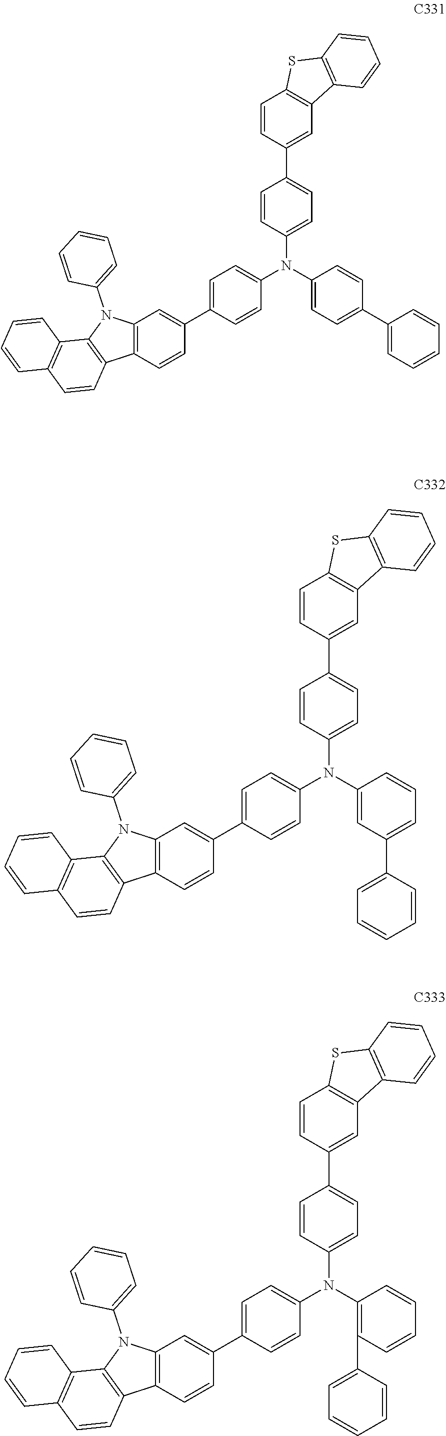 [SMILES: c1ccc(-c2ccc(N(c3ccc(-c4ccc5sc6ccccc6c5c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5sc6ccccc6c5c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4sc5ccccc5c4c3)cc2)c2ccc(-c3ccc4c5ccc6ccccc6c5n(-c5ccccc5)c4c3)cc2)cc1]

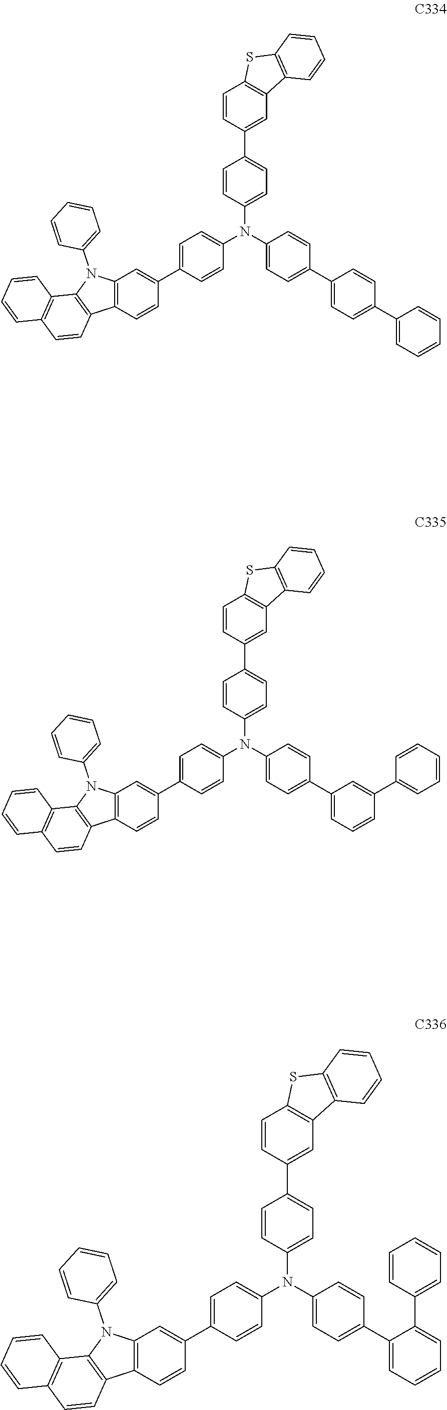 [SMILES: c1ccc(-c2ccc(-c3ccc(N(c4ccc(-c5ccc6sc7ccccc7c6c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6sc7ccccc7c6c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5sc6ccccc6c5c4)cc3)c3ccc(-c4ccc5c6ccc7ccccc7c6n(-c6ccccc6)c5c4)cc3)cc2)cc1]

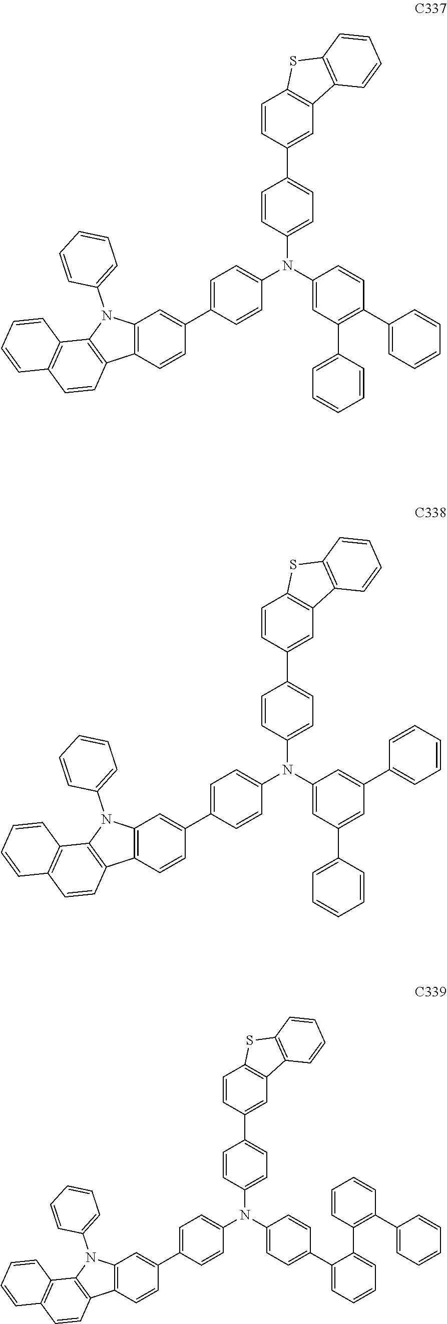 [SMILES: c1ccc(-c2ccc(N(c3ccc(-c4ccc5sc6ccccc6c5c4)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5sc6ccccc6c5c4)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5sc6ccccc6c5c4)cc3)c3ccc(-c4ccc5c6ccc7ccccc7c6n(-c6ccccc6)c5c4)cc3)cc2)cc1]

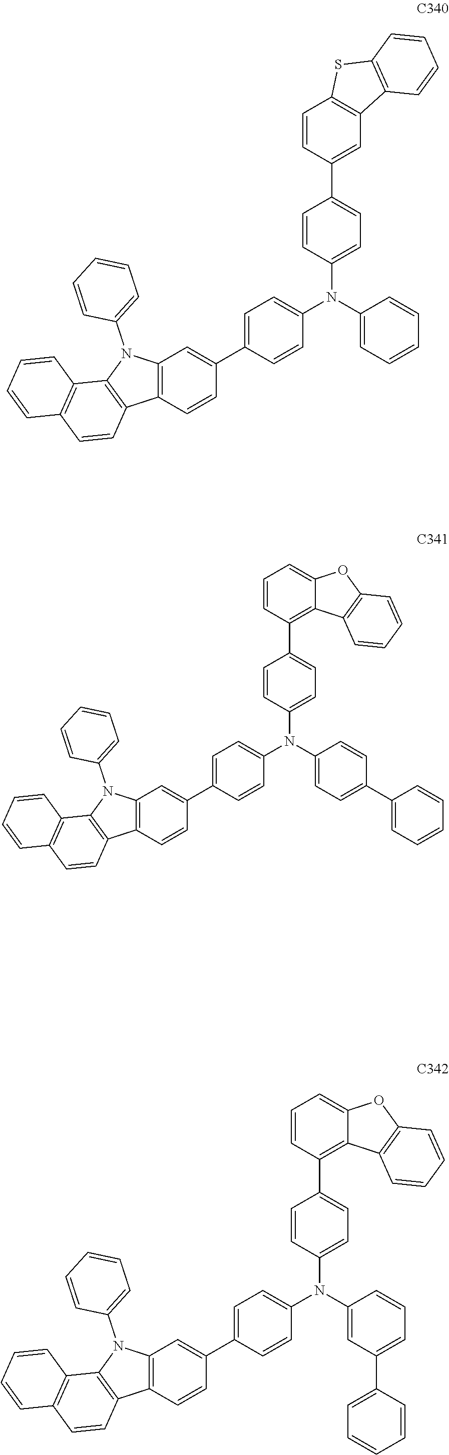 [SMILES: c1ccc(N(c2ccc(-c3ccc4sc5ccccc5c4c3)cc2)c2ccc(-c3ccc4c5ccc6ccccc6c5n(-c5ccccc5)c4c3)cc2)cc1 ; c1ccc(-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1 ; c1ccc(-c2cccc(N(c3ccc(-c4ccc5c6ccc7ccccc7c6n(-c6ccccc6)c5c4)cc3)c3ccc(-c4cccc5oc6ccccc6c45)cc3)c2)cc1]

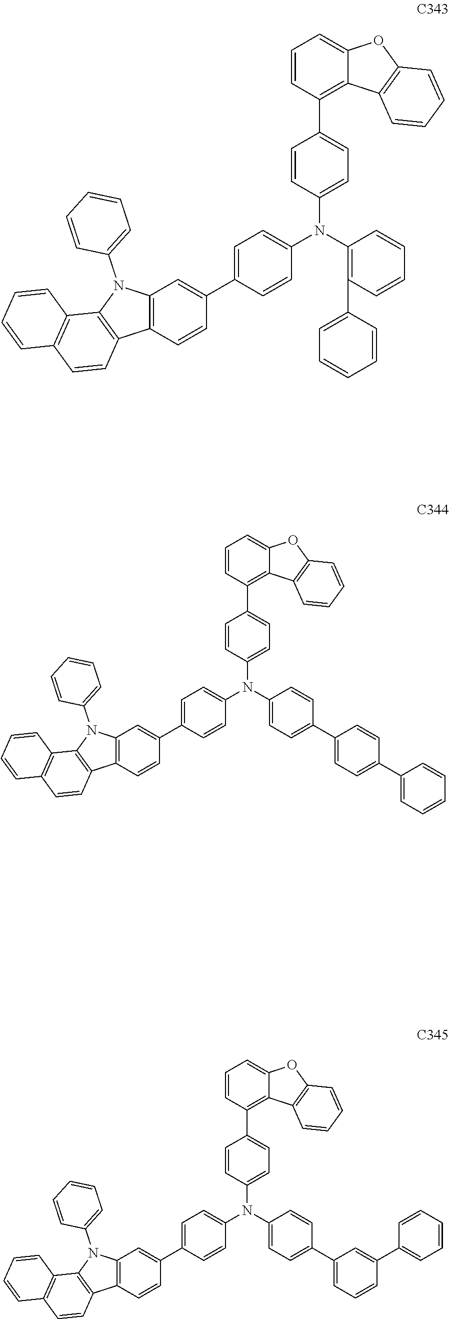 [SMILES: c1ccc(-c2ccccc2N(c2ccc(-c3ccc4c5ccc6ccccc6c5n(-c5ccccc5)c4c3)cc2)c2ccc(-c3cccc4oc5ccccc5c34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6oc7ccccc7c56)cc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc(-c5cccc6oc7ccccc7c56)cc4)cc3)c2)cc1]

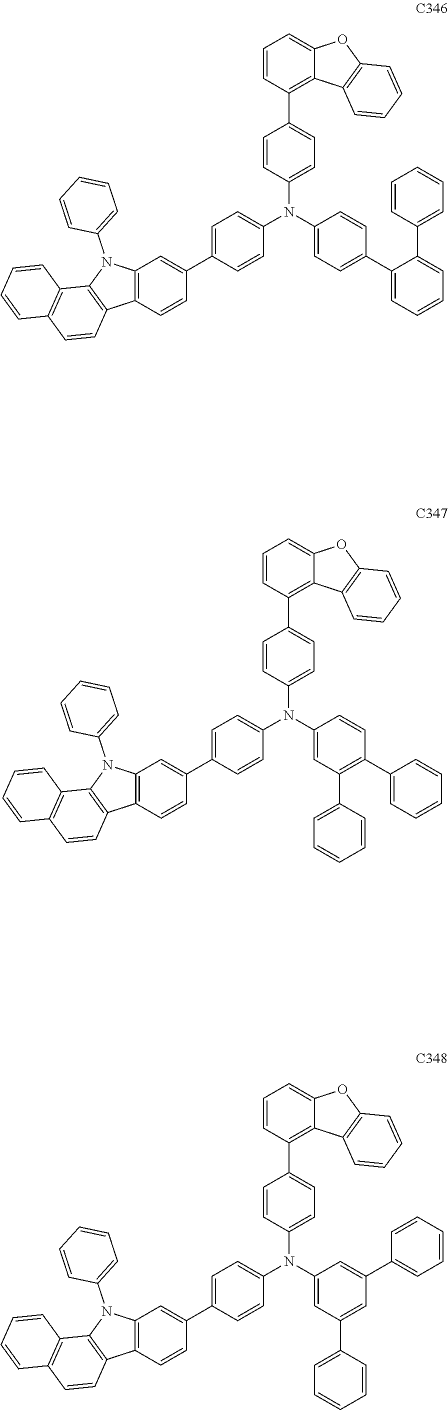 [SMILES: c1ccc(-c2ccccc2-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1 ; c1ccc(-c2ccc(N(c3ccc(-c4ccc5c6ccc7ccccc7c6n(-c6ccccc6)c5c4)cc3)c3ccc(-c4cccc5oc6ccccc6c45)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cccc5oc6ccccc6c45)cc3)c2)cc1]

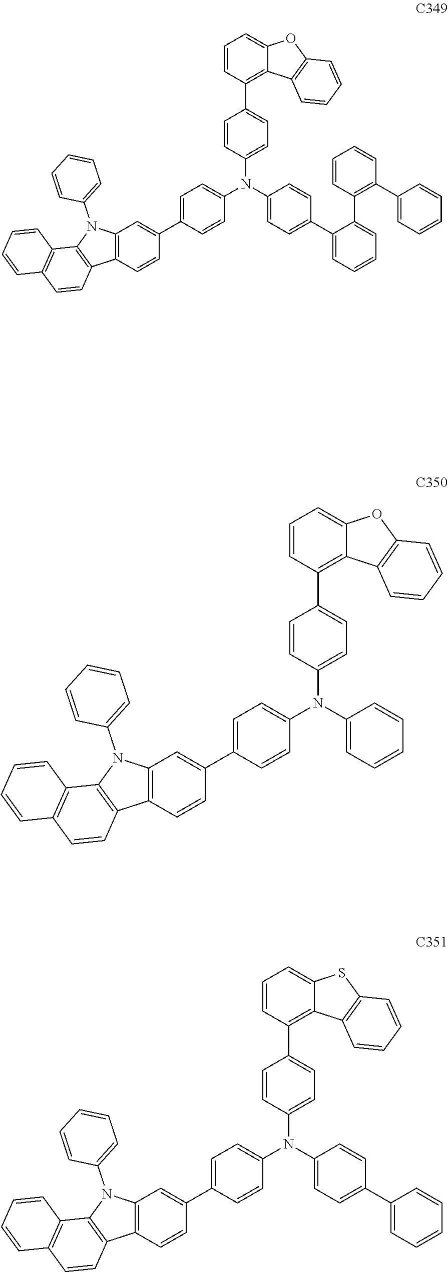 [SMILES: c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1 ; c1ccc(N(c2ccc(-c3ccc4c5ccc6ccccc6c5n(-c5ccccc5)c4c3)cc2)c2ccc(-c3cccc4oc5ccccc5c34)cc2)cc1 ; c1ccc(-c2ccc(N(c3ccc(-c4ccc5c6ccc7ccccc7c6n(-c6ccccc6)c5c4)cc3)c3ccc(-c4cccc5sc6ccccc6c45)cc3)cc2)cc1]

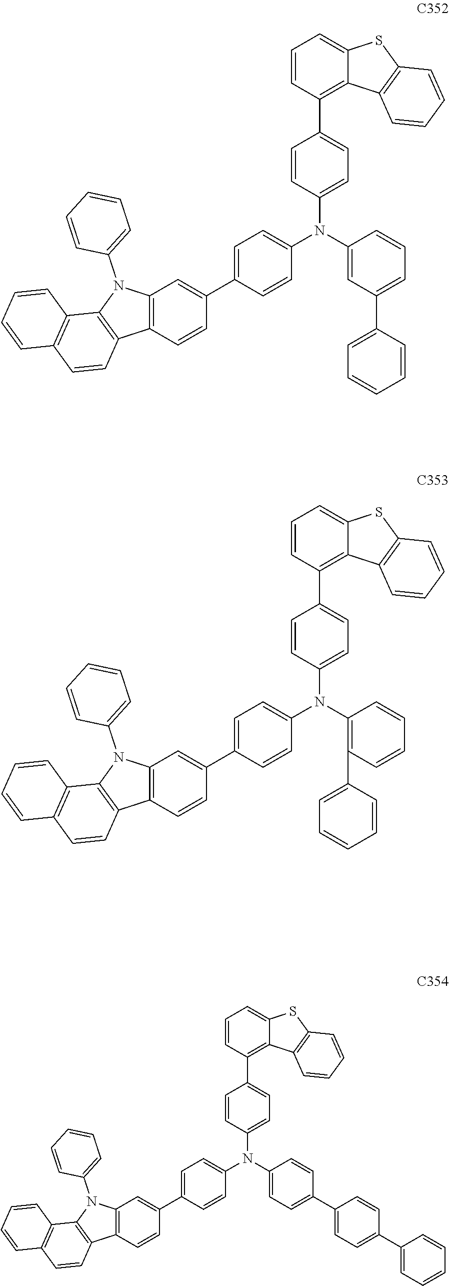 [SMILES: c1ccc(-c2cccc(N(c3ccc(-c4ccc5c6ccc7ccccc7c6n(-c6ccccc6)c5c4)cc3)c3ccc(-c4cccc5sc6ccccc6c45)cc3)c2)cc1 ; c1ccc(-c2ccccc2N(c2ccc(-c3ccc4c5ccc6ccccc6c5n(-c5ccccc5)c4c3)cc2)c2ccc(-c3cccc4sc5ccccc5c34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6sc7ccccc7c56)cc4)cc3)cc2)cc1]

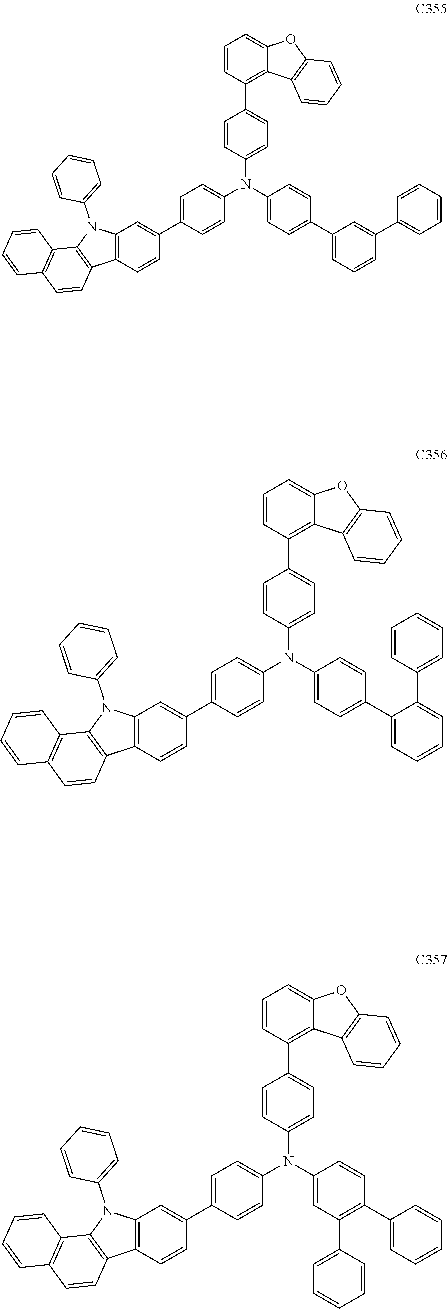 [SMILES: c1ccc(-c2cccc(-c3ccc(N(c4ccc(-c5ccc6c7ccc8ccccc8c7n(-c7ccccc7)c6c5)cc4)c4ccc(-c5cccc6oc7ccccc7c56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1 ; c1ccc(-c2ccc(N(c3ccc(-c4ccc5c6ccc7ccccc7c6n(-c6ccccc6)c5c4)cc3)c3ccc(-c4cccc5oc6ccccc6c45)cc3)cc2-c2ccccc2)cc1]

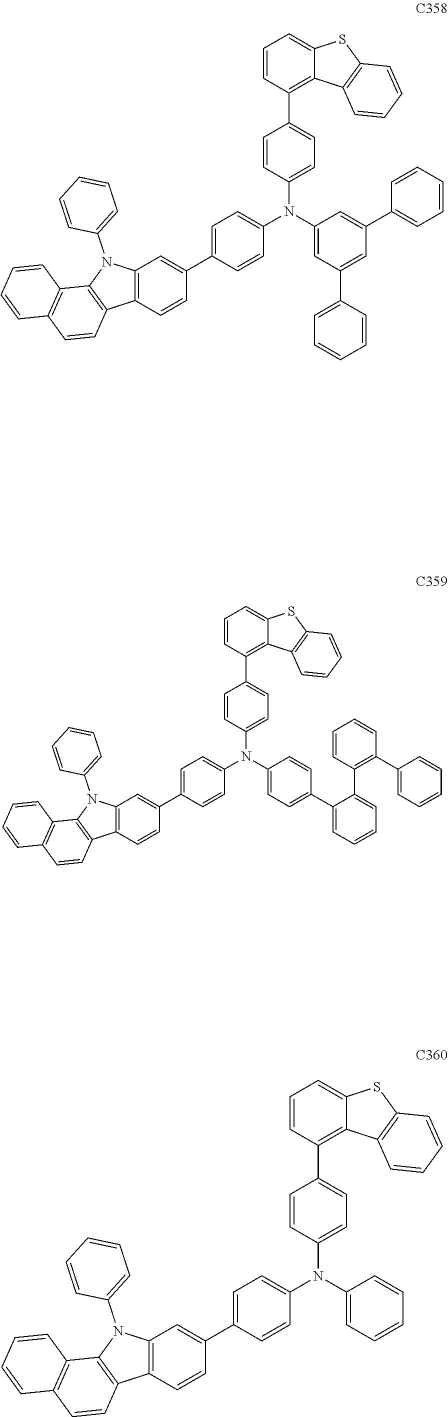 [SMILES: c1ccc(-c2cc(-c3ccccc3)cc(N(c3ccc(-c4ccc5c6ccc7ccccc7c6n(-c6ccccc6)c5c4)cc3)c3ccc(-c4cccc5sc6ccccc6c45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5sc6ccccc6c45)cc3)cc2)cc1 ; c1ccc(N(c2ccc(-c3ccc4c5ccc6ccccc6c5n(-c5ccccc5)c4c3)cc2)c2ccc(-c3cccc4sc5ccccc5c34)cc2)cc1]

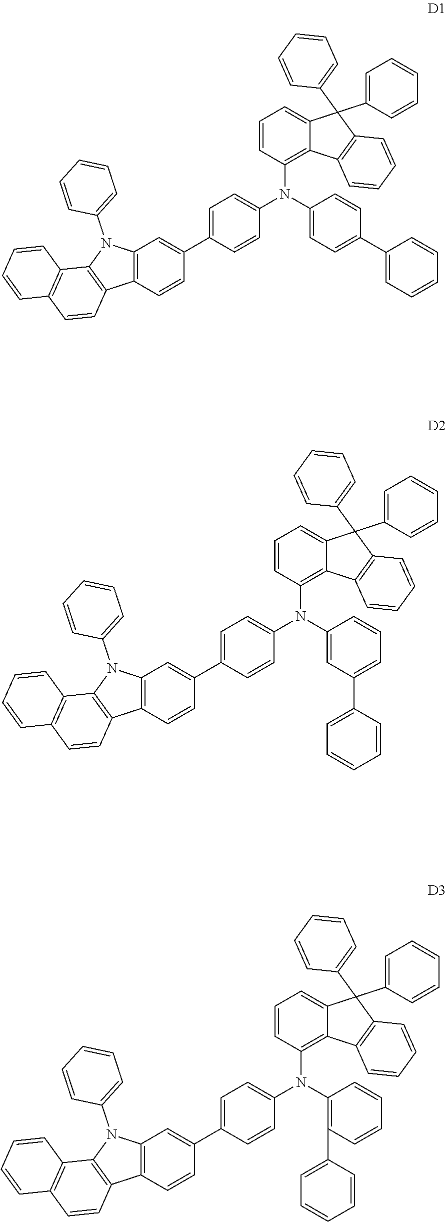 [SMILES: c1ccc(-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(-c2cccc(N(c3ccc(-c4ccc5c6ccc7ccccc7c6n(-c6ccccc6)c5c4)cc3)c3cccc4c3-c3ccccc3C4(c3ccccc3)c3ccccc3)c2)cc1 ; c1ccc(-c2ccccc2N(c2ccc(-c3ccc4c5ccc6ccccc6c5n(-c5ccccc5)c4c3)cc2)c2cccc3c2-c2ccccc2C3(c2ccccc2)c2ccccc2)cc1]

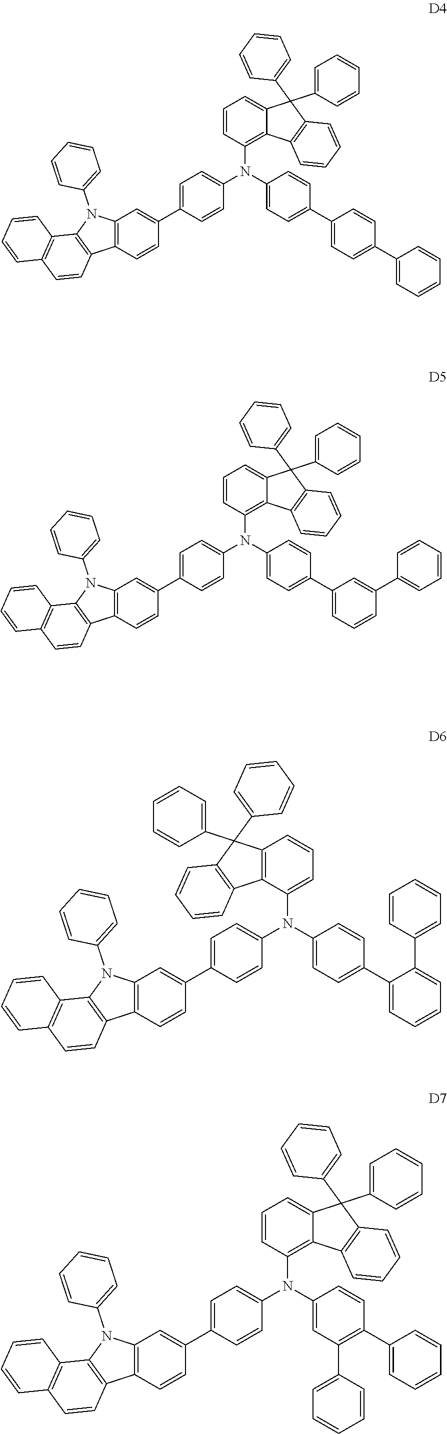 [SMILES: c1ccc(-c2ccc(-c3ccc(N(c4ccc(-c5ccc6c7ccc8ccccc8c7n(-c7ccccc7)c6c5)cc4)c4cccc5c4-c4ccccc4C5(c4ccccc4)c4ccccc4)cc3)cc2)cc1 ; c1ccc(-c2cccc(-c3ccc(N(c4ccc(-c5ccc6c7ccc8ccccc8c7n(-c7ccccc7)c6c5)cc4)c4cccc5c4-c4ccccc4C5(c4ccccc4)c4ccccc4)cc3)c2)cc1 ; c1ccc(-c2ccccc2-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2-c2ccccc2)cc1]

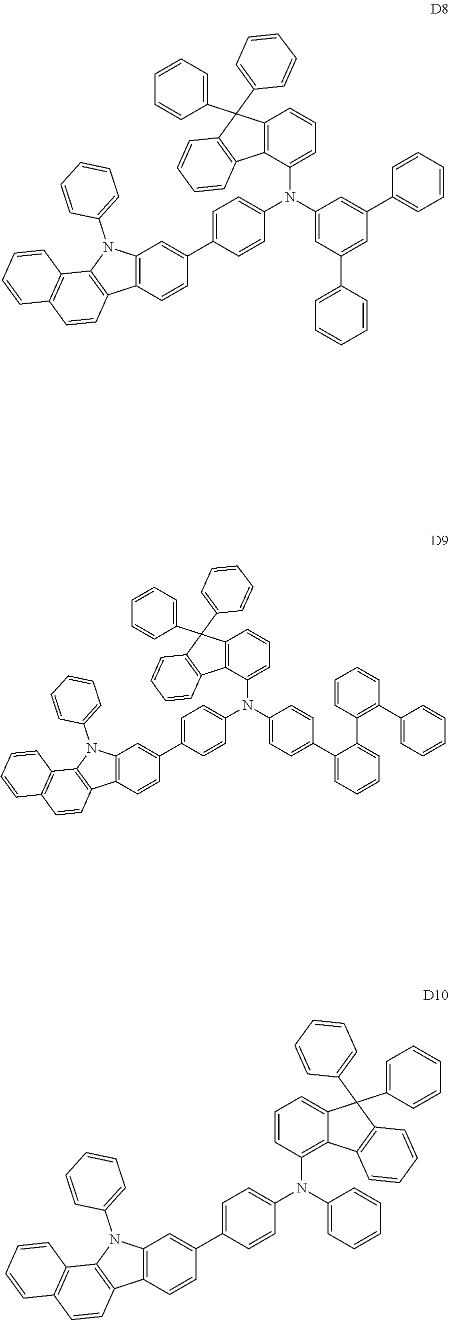 [SMILES: c1ccc(-c2cc(-c3ccccc3)cc(N(c3ccc(-c4ccc5c6ccc7ccccc7c6n(-c6ccccc6)c5c4)cc3)c3cccc4c3-c3ccccc3C4(c3ccccc3)c3ccccc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(N(c2ccc(-c3ccc4c5ccc6ccccc6c5n(-c5ccccc5)c4c3)cc2)c2cccc3c2-c2ccccc2C3(c2ccccc2)c2ccccc2)cc1]

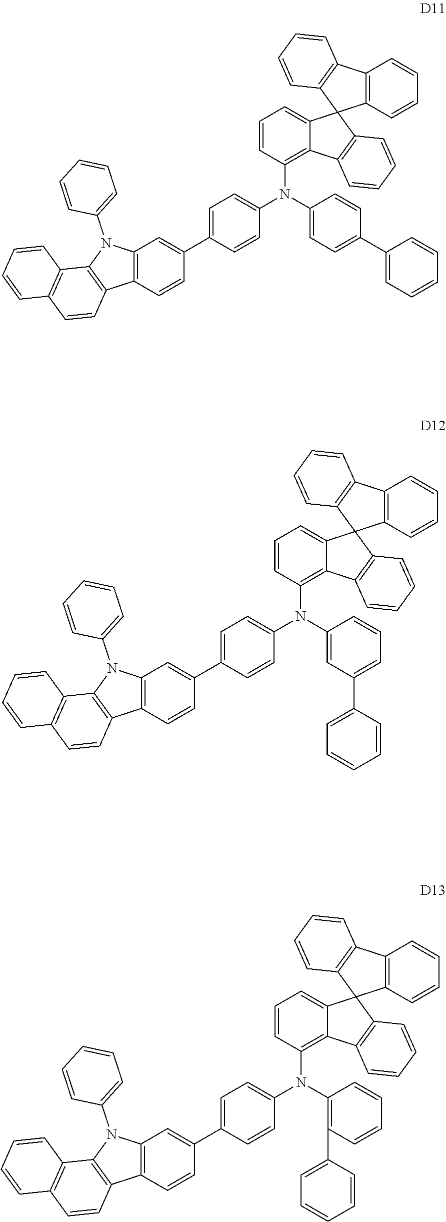 [SMILES: c1ccc(-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1 ; c1ccc(-c2cccc(N(c3ccc(-c4ccc5c6ccc7ccccc7c6n(-c6ccccc6)c5c4)cc3)c3cccc4c3-c3ccccc3C43c4ccccc4-c4ccccc43)c2)cc1 ; c1ccc(-c2ccccc2N(c2ccc(-c3ccc4c5ccc6ccccc6c5n(-c5ccccc5)c4c3)cc2)c2cccc3c2-c2ccccc2C32c3ccccc3-c3ccccc32)cc1]

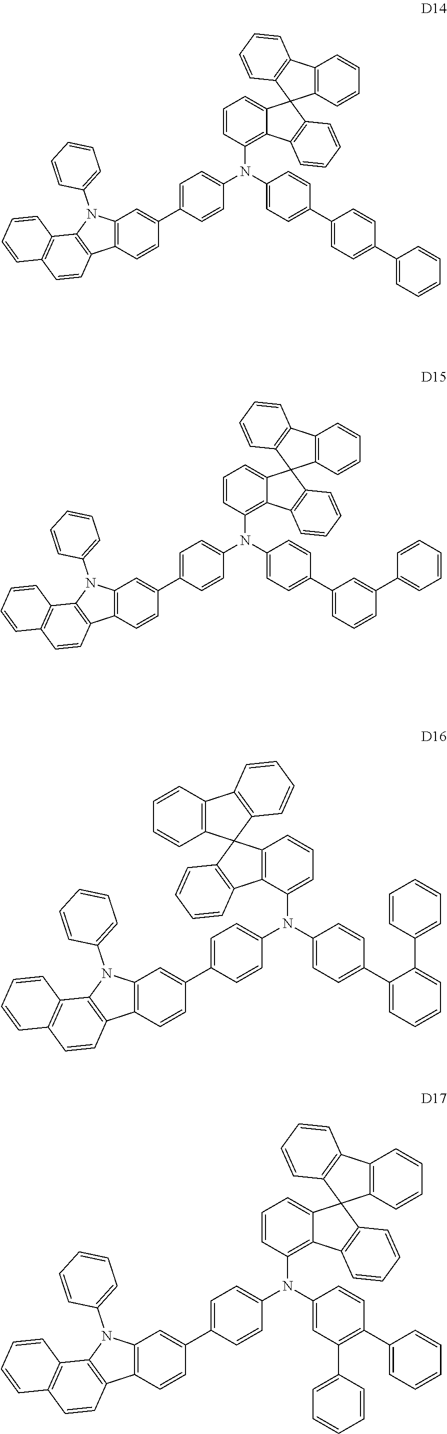 [SMILES: c1ccc(-c2ccc(-c3ccc(N(c4ccc(-c5ccc6c7ccc8ccccc8c7n(-c7ccccc7)c6c5)cc4)c4cccc5c4-c4ccccc4C54c5ccccc5-c5ccccc54)cc3)cc2)cc1 ; c1ccc(-c2cccc(-c3ccc(N(c4ccc(-c5ccc6c7ccc8ccccc8c7n(-c7ccccc7)c6c5)cc4)c4cccc5c4-c4ccccc4C54c5ccccc5-c5ccccc54)cc3)c2)cc1 ; c1ccc(-c2ccccc2-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1 ; c1ccc(-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2-c2ccccc2)cc1]

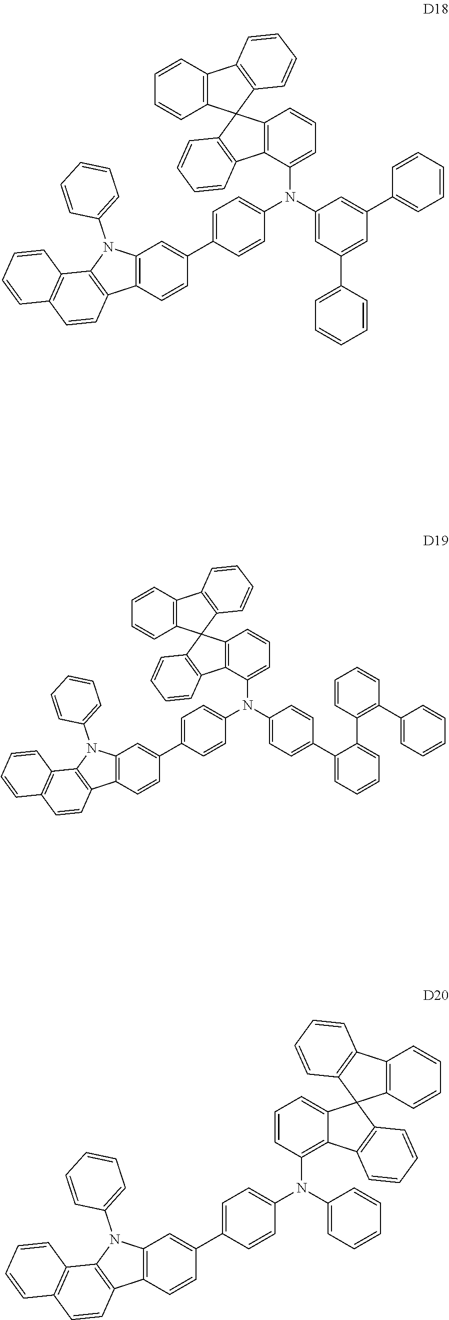 [SMILES: c1ccc(-c2cc(-c3ccccc3)cc(N(c3ccc(-c4ccc5c6ccc7ccccc7c6n(-c6ccccc6)c5c4)cc3)c3cccc4c3-c3ccccc3C43c4ccccc4-c4ccccc43)c2)cc1 ; c1ccc(-c2ccccc2-c2ccccc2-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1 ; c1ccc(N(c2ccc(-c3ccc4c5ccc6ccccc6c5n(-c5ccccc5)c4c3)cc2)c2cccc3c2-c2ccccc2C32c3ccccc3-c3ccccc32)cc1]

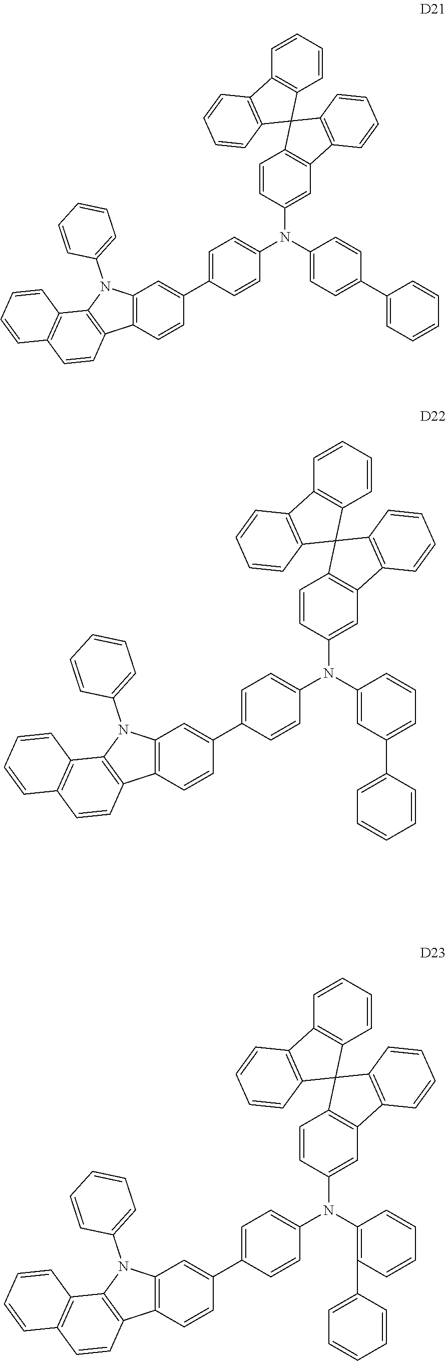 [SMILES: c1ccc(-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2)cc1 ; c1ccc(-c2cccc(N(c3ccc(-c4ccc5c6ccc7ccccc7c6n(-c6ccccc6)c5c4)cc3)c3ccc4c(c3)-c3ccccc3C43c4ccccc4-c4ccccc43)c2)cc1 ; c1ccc(-c2ccccc2N(c2ccc(-c3ccc4c5ccc6ccccc6c5n(-c5ccccc5)c4c3)cc2)c2ccc3c(c2)-c2ccccc2C32c3ccccc3-c3ccccc32)cc1]

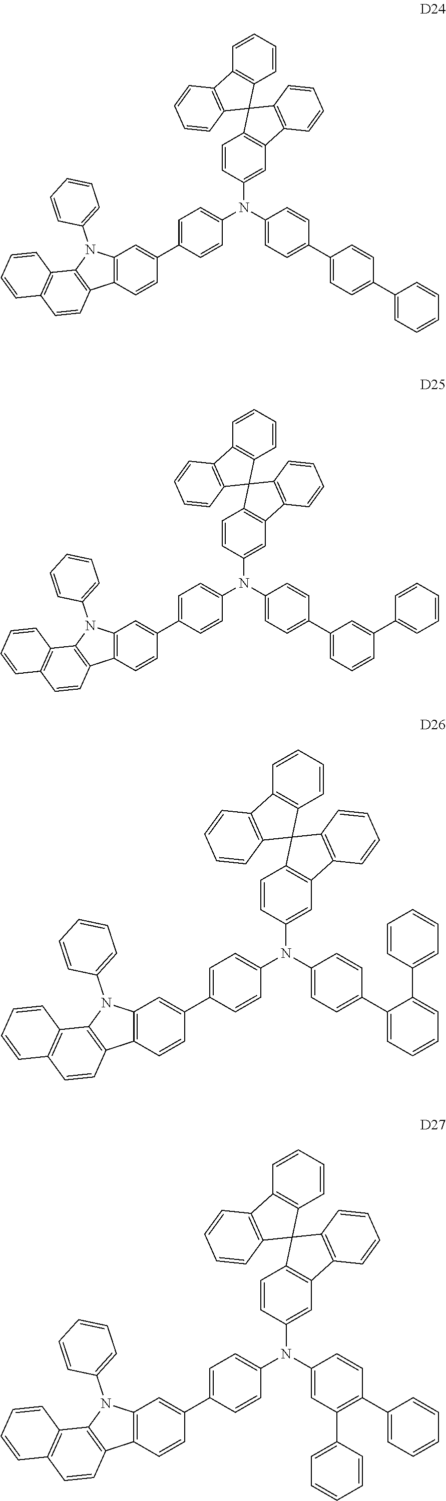 [SMILES: c1ccc(-c2ccc(-c3ccc(N(c4ccc(-c5ccc6c7ccc8ccccc8c7n(-c7ccccc7)c6c5)cc4)c4ccc5c(c4)-c4ccccc4C54c5ccccc5-c5ccccc54)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)-c4ccccc4C54c5ccccc5-c5ccccc54)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2)cc1 ; c1ccc(-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2-c2ccccc2)cc1]

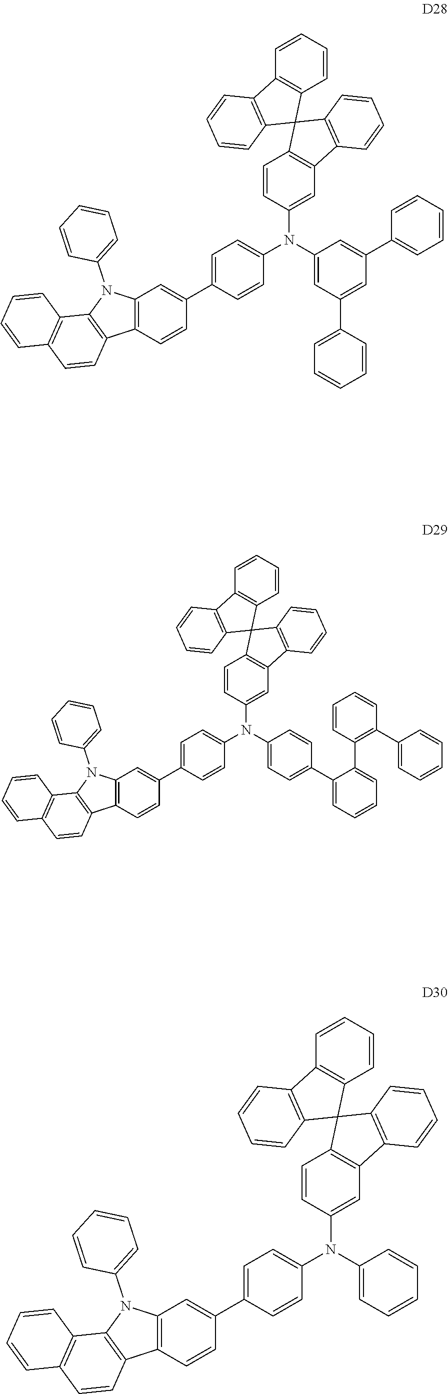 [SMILES: c1ccc(-c2cc(-c3ccccc3)cc(N(c3ccc(-c4ccc5c6ccc7ccccc7c6n(-c6ccccc6)c5c4)cc3)c3ccc4c(c3)-c3ccccc3C43c4ccccc4-c4ccccc43)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2)cc1 ; c1ccc(N(c2ccc(-c3ccc4c5ccc6ccccc6c5n(-c5ccccc5)c4c3)cc2)c2ccc3c(c2)-c2ccccc2C32c3ccccc3-c3ccccc32)cc1]

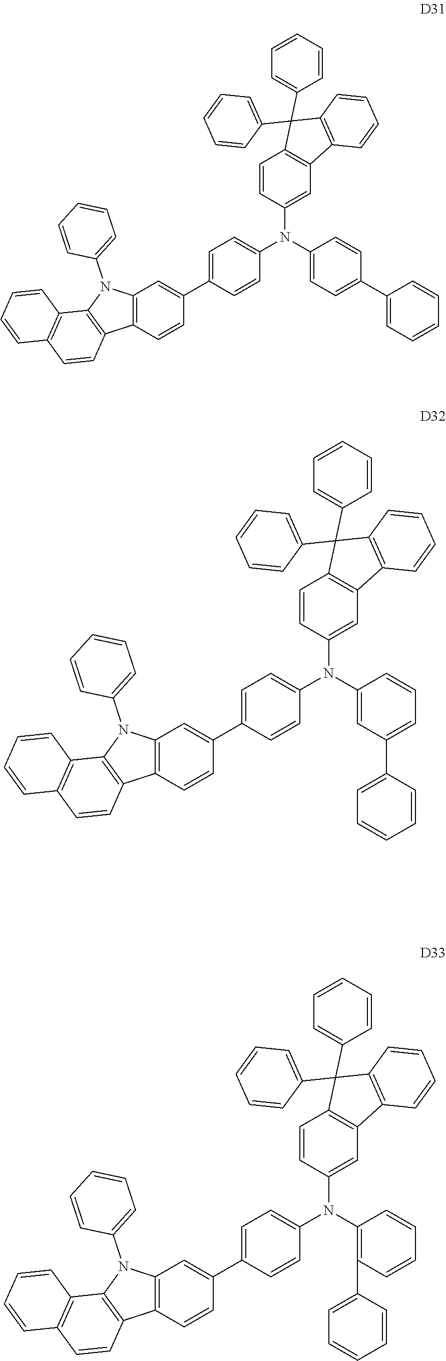 [SMILES: c1ccc(-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2)cc1 ; c1ccc(-c2cccc(N(c3ccc(-c4ccc5c6ccc7ccccc7c6n(-c6ccccc6)c5c4)cc3)c3ccc4c(c3)-c3ccccc3C4(c3ccccc3)c3ccccc3)c2)cc1 ; c1ccc(-c2ccccc2N(c2ccc(-c3ccc4c5ccc6ccccc6c5n(-c5ccccc5)c4c3)cc2)c2ccc3c(c2)-c2ccccc2C3(c2ccccc2)c2ccccc2)cc1]

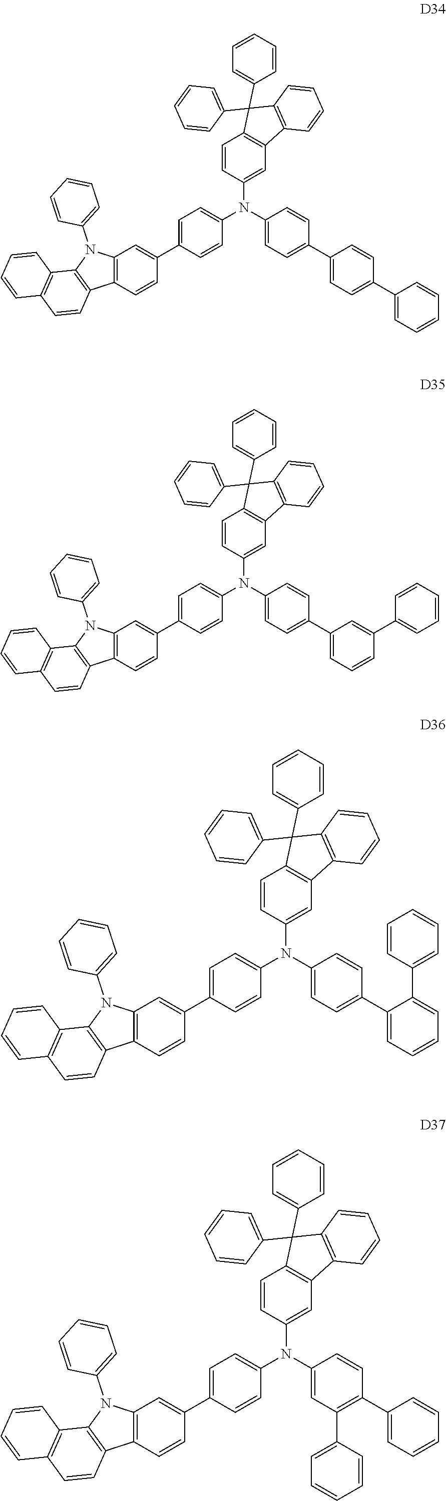 [SMILES: c1ccc(-c2ccc(-c3ccc(N(c4ccc(-c5ccc6c7ccc8ccccc8c7n(-c7ccccc7)c6c5)cc4)c4ccc5c(c4)-c4ccccc4C5(c4ccccc4)c4ccccc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)-c4ccccc4C5(c4ccccc4)c4ccccc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2)cc1 ; c1ccc(-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2-c2ccccc2)cc1]

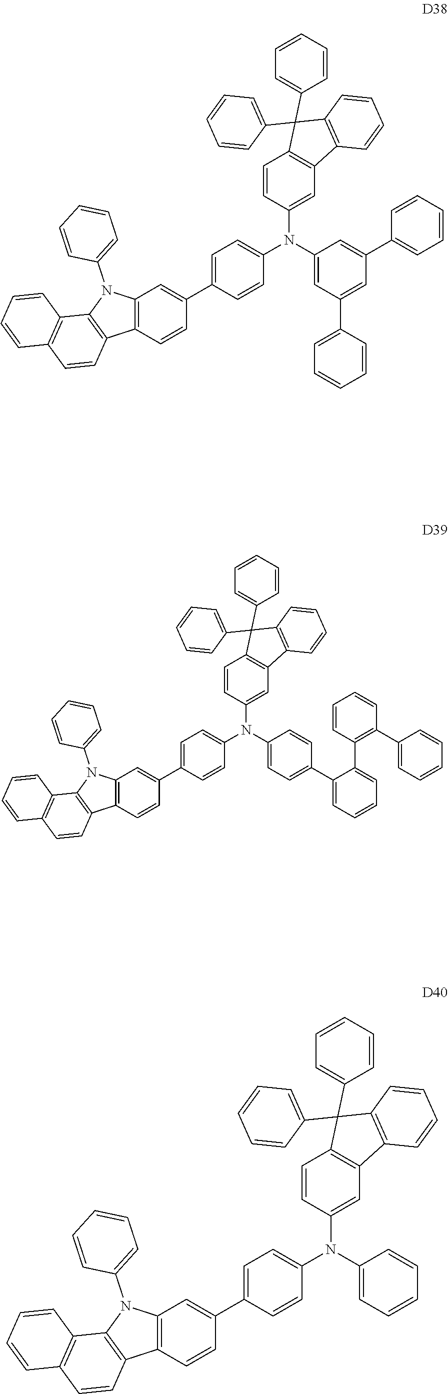 [SMILES: c1ccc(-c2cc(-c3ccccc3)cc(N(c3ccc(-c4ccc5c6ccc7ccccc7c6n(-c6ccccc6)c5c4)cc3)c3ccc4c(c3)-c3ccccc3C4(c3ccccc3)c3ccccc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2)cc1 ; c1ccc(N(c2ccc(-c3ccc4c5ccc6ccccc6c5n(-c5ccccc5)c4c3)cc2)c2ccc3c(c2)-c2ccccc2C3(c2ccccc2)c2ccccc2)cc1]

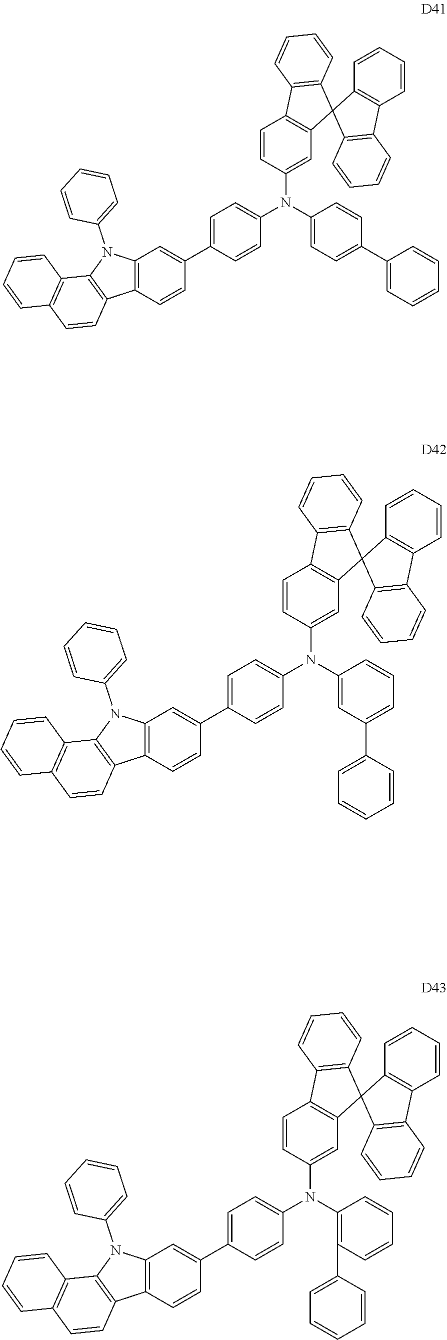 [SMILES: c1ccc(-c2ccc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)cc2)cc1 ; c1ccc(-c2cccc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)c2)cc1 ; c1ccc(-c2ccccc2N(c2ccc(-c3ccc4c5ccc6ccccc6c5n(-c5ccccc5)c4c3)cc2)c2ccc3c(c2)C2(c4ccccc4-c4ccccc42)c2ccccc2-3)cc1]

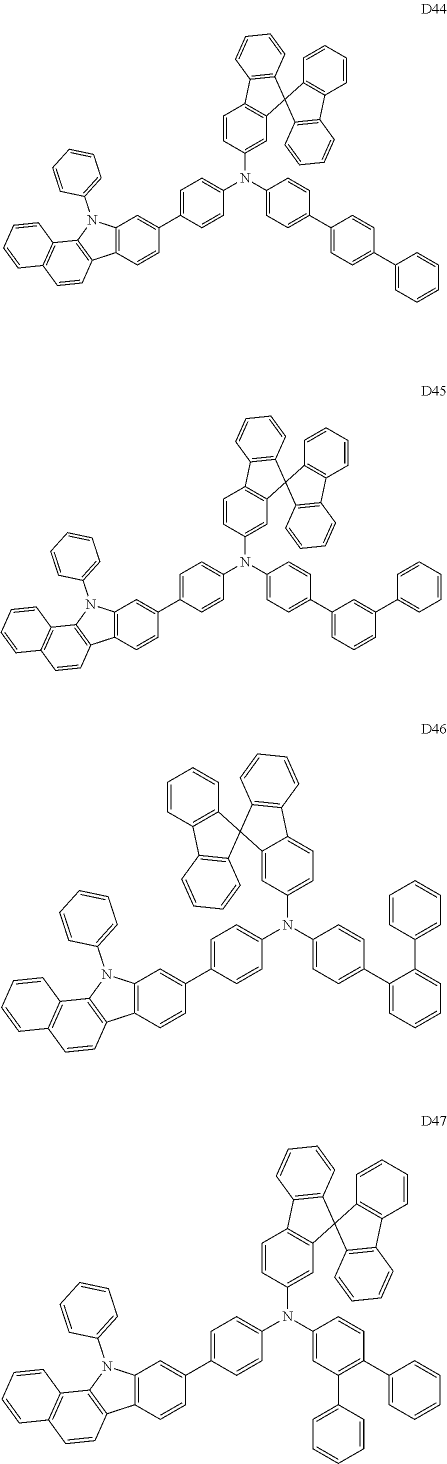

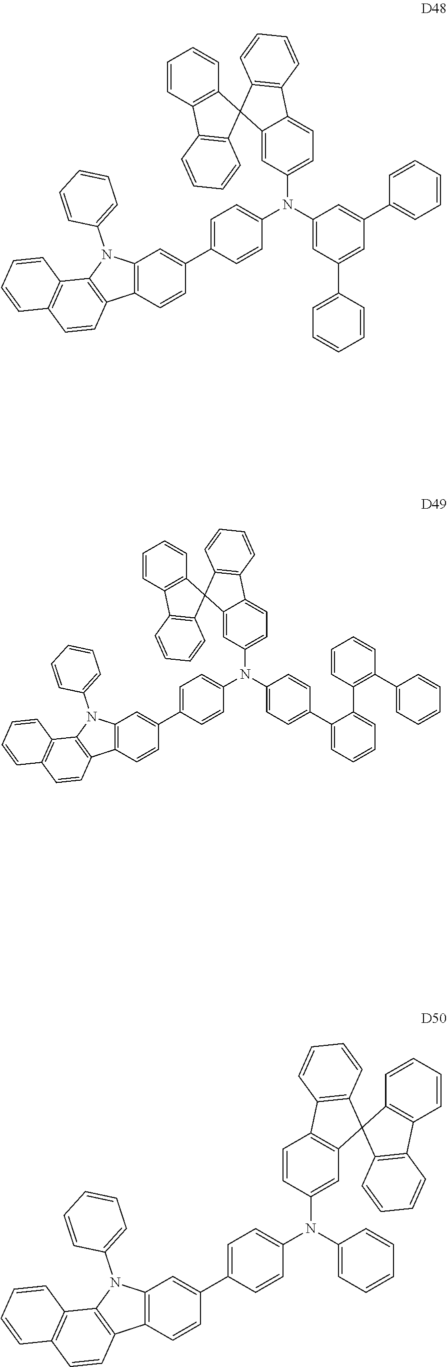 [SMILES: c1ccc(-c2cc(-c3ccccc3)cc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)cc2)cc1 ; c1ccc(N(c2ccc(-c3ccc4c5ccc6ccccc6c5n(-c5ccccc5)c4c3)cc2)c2ccc3c(c2)C2(c4ccccc4-c4ccccc42)c2ccccc2-3)cc1]

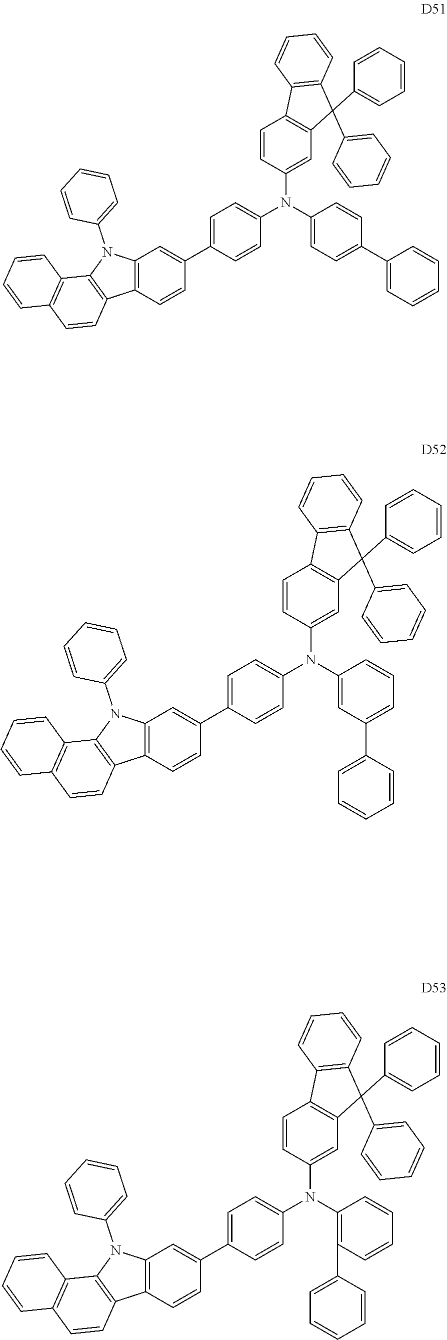 [SMILES: c1ccc(-c2ccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)cc2)cc1 ; c1ccc(-c2cccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)c2)cc1 ; c1ccc(-c2ccccc2N(c2ccc(-c3ccc4c5ccc6ccccc6c5n(-c5ccccc5)c4c3)cc2)c2ccc3c(c2)C(c2ccccc2)(c2ccccc2)c2ccccc2-3)cc1]

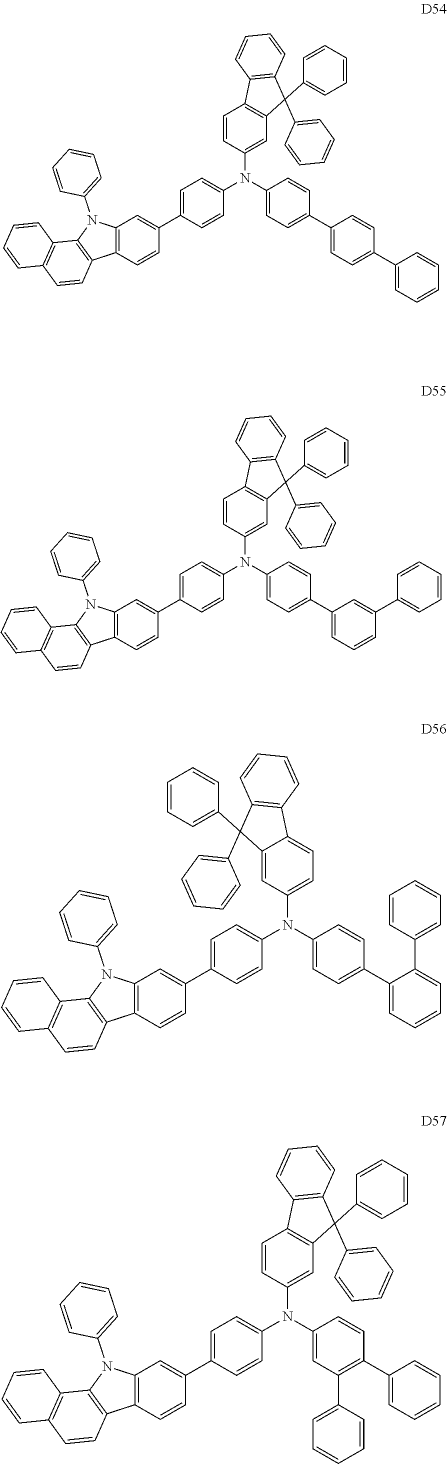

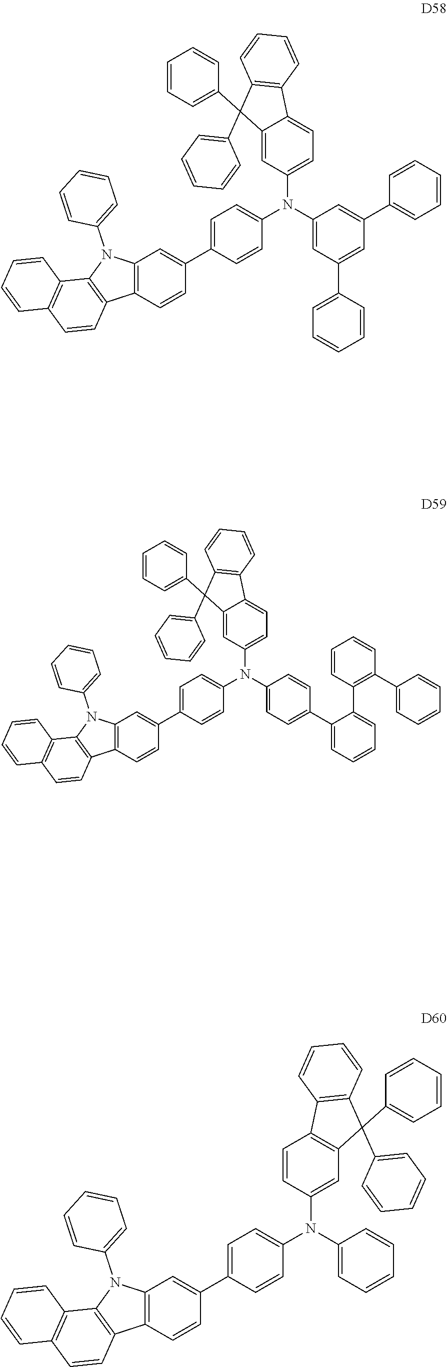 [SMILES: c1ccc(-c2cc(-c3ccccc3)cc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)cc2)cc1 ; c1ccc(N(c2ccc(-c3ccc4c5ccc6ccccc6c5n(-c5ccccc5)c4c3)cc2)c2ccc3c(c2)C(c2ccccc2)(c2ccccc2)c2ccccc2-3)cc1]

The amine compound represented by Formula 1 may contain a benzocarbazole moiety, and may contain, in an amine moiety, at least one substituent of a substituted or unsubstituted naphthyl group, a substituted or unsubstituted phenanthryl group, a substituted or unsubstituted benzoheterole group, or a substituted fluorenyl group. Accordingly, the amine compound may have an improvement in electron resistance and exciton resistance, and the embodiment in which the amine compound is used as a material for forming a light emitting element may contribute to a reduced driving voltage and high efficiency of the light emitting element. Accordingly, the light emitting element including the amine compound may exhibit a high efficiency and long service life characteristics.

In some embodiments, when the light emitting element ED includes a plurality of hole transport layers, the hole transport layer adjacent to the emission layer among the plurality of hole transport layers may include the above-described amine compound.

In some embodiments, the light emitting element ED may further include materials for the hole transport region, described below, in the hole transport region HTR in addition to the above-described amine compound.

The hole transport region HTR may include a compound represented by Formula H-1:

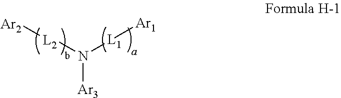

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms; and a and b may be each independently an integer from 0 to 10. In some embodiments, when a or b is an integer of 2 or greater, a plurality of L₁s and L₂s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ara may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound. Alternatively, the compound represented by Formula H-1 may be a diamine compound in which at least one selected from among Ar₁ to Ar₃ includes the amine group as a substituent. In some embodiments, the compound represented by Formula H-1 may be a carbazole-based compound including a substituted or unsubstituted carbazole group in at least one selected from Ar₁ and Ar₂, or a fluorene-based compound including a substituted or unsubstituted fluorene group in at least one selected from Ar₁ and Ar₂.

The compound represented by Formula H-1 may be represented by one selected from among the compounds of Compound Group H. However, the compounds listed in Compound Group H are examples, and the compounds represented by Formula H-1 are not limited to those represented by Compound Group H:

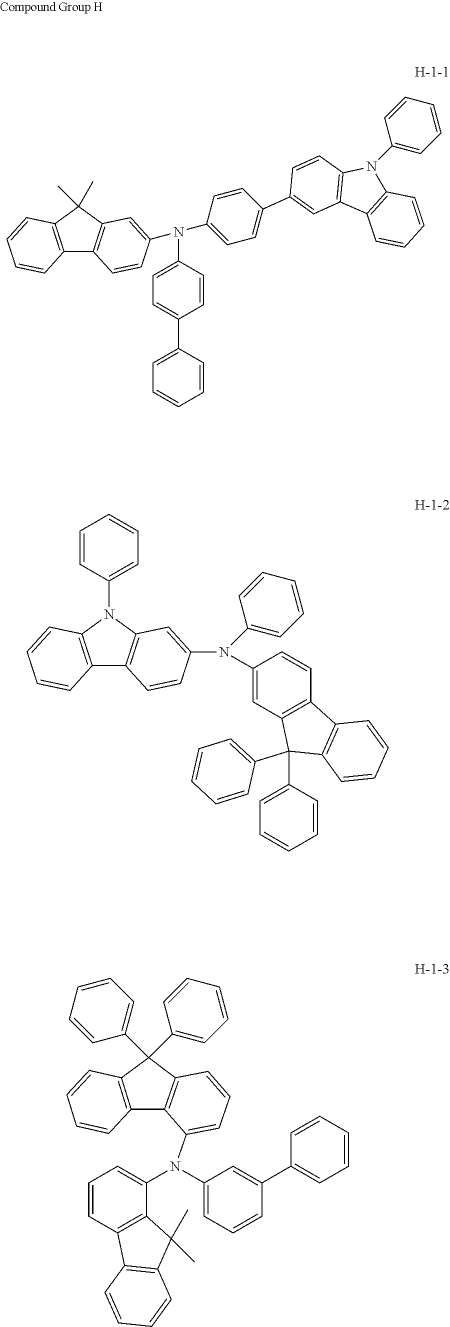 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

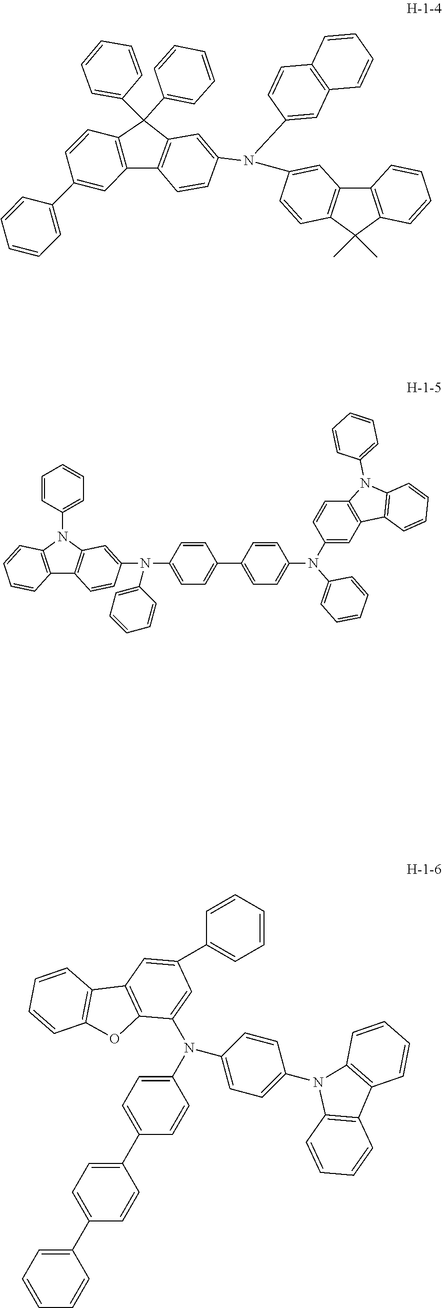 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

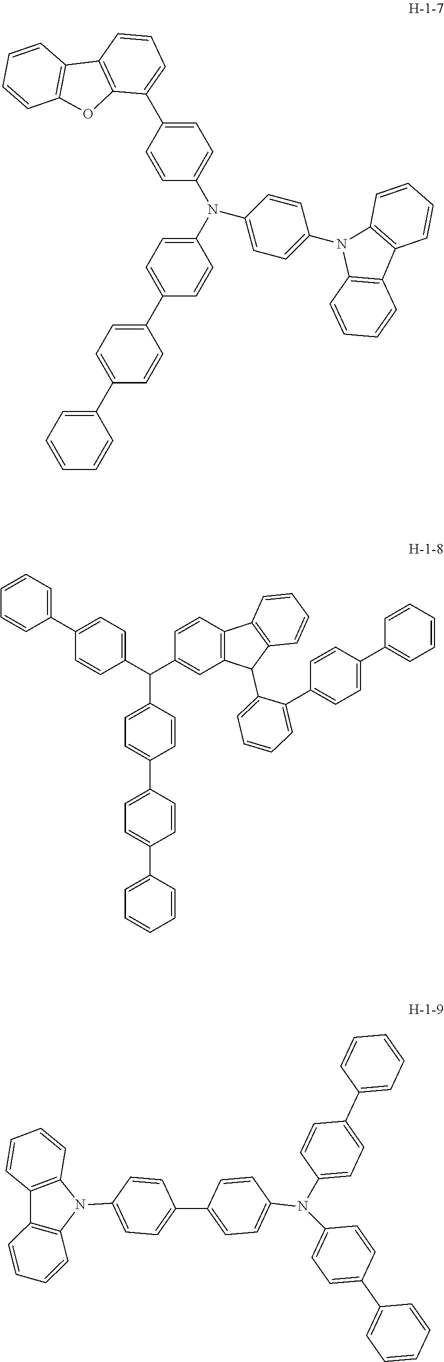

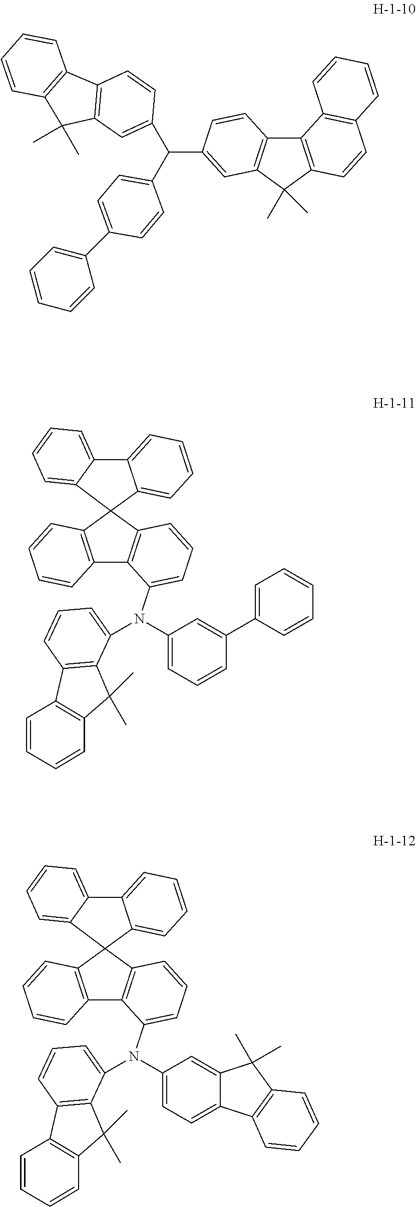

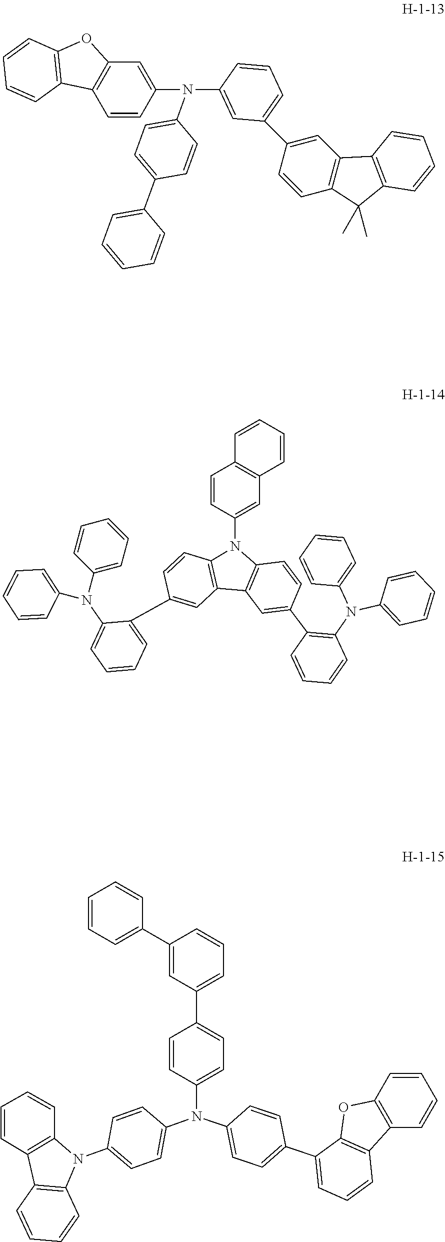 [SMILES: CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

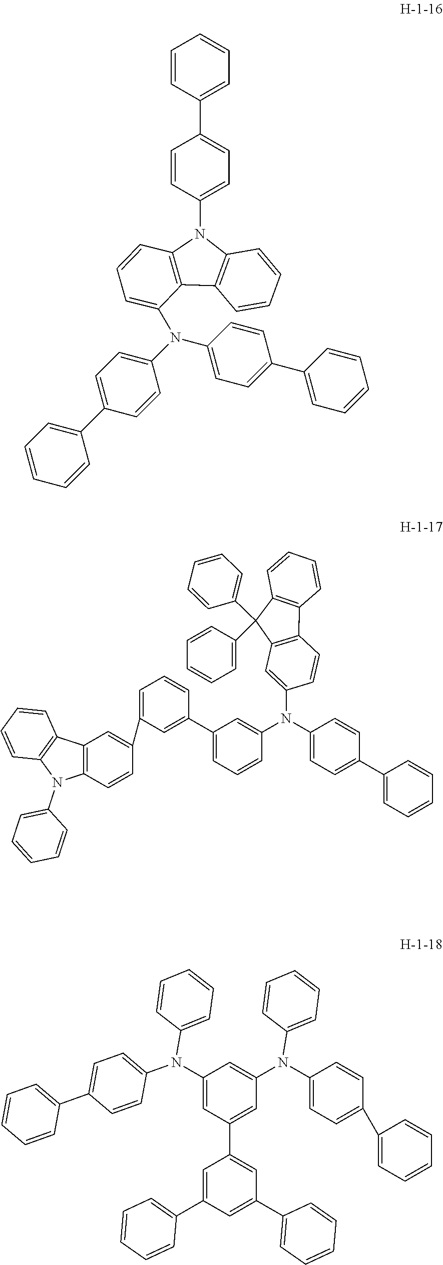 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

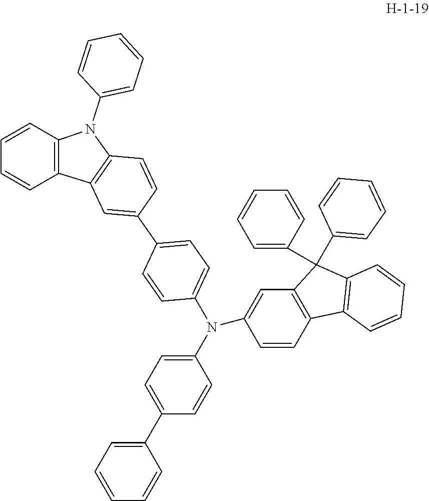 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may further include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-l-yl)-N,N′-diphenyl-benzidine (NPB (or NPD)), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalen-l-yl)-N,N′-diphenyl-benzidine (NPB), 4,4′-cyclohexylidene bis[N,N-bis(4-methylphenyl]benzenamine] (TAPC), 4,4′-bis[N,N-(3-tolyl)amino]-3,3′-dimethylbiphenyl (HMTPD), 1,3-bis(N-carbazolyl)benzene (mCP), etc.

In addition, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one selected from a hole injection layer HIL, a hole transport layer HTL, and an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 30 Å to about 1,000 Å. For example, when the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the above-described ranges, satisfactory (suitable) hole transport properties may be achieved without a substantial increase in driving voltage.

The hole transport region HTR may further include a charge generating material to increase conductivity in addition to the above-described materials. The charge generating material may be dispersed substantially uniformly or non-uniformly in the hole transport region HTR. The charge generating material may be, for example, a p-dopant. The p-dopant may include at least one selected from a halogenated metal compound, a quinone derivative, a metal oxide, and a cyano group-containing compound, but the embodiment of the present disclosure is not limited thereto. For example, the p-dopant may include a metal halide compound such as CuI or RbI, a quinone derivative such as tetracyanoquinodimethane (TCNQ) or 2,3,5,6-tetrafluoro-7,7, 8,8-tetracyanoquinodimethane (F4-TCNQ), a metal oxide such as tungsten oxide or molybdenum oxide, a cyano group-containing compound such as dipyrazino[2,3-f: 2′,3′-h] quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but the embodiment of the present disclosure is not limited thereto.

As described above, the hole transport region HTR may further include at least one selected from the buffer layer and the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be contained in the hole transport region HTR may also be used as a material to be contained in the buffer layer. The electron blocking layer EBL is a layer that serves to prevent (reduce) the electron injection from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

The emission layer EML in the light emitting element ED of an embodiment may emit one light selected from among red light, green light, blue light, white light, and cyan light. The light emitting element ED of an embodiment may include the above-described amine compound in the hole transport region HTR, thereby exhibiting high efficiency and long service life characteristics in the light emitting region for emitting the light. However, the embodiment of the present disclosure is not limited thereto.

In the light emitting element ED, the emission layer EML may include an anthracene derivative, a pyrene derivative, a fluoranthene derivative, a chrysene derivative, a dehydrobenzanthracene derivative, or a triphenylene derivative. For example, the emission layer EML may include the anthracene derivative or the pyrene derivative.

In each light emitting element ED of embodiments illustrated in FIGS. 3 to 6, the emission layer EML may include a host and a dopant, and the emission layer EML may include a compound represented by Formula E-1. The compound represented by Formula E-1 may be used as a fluorescent host material.

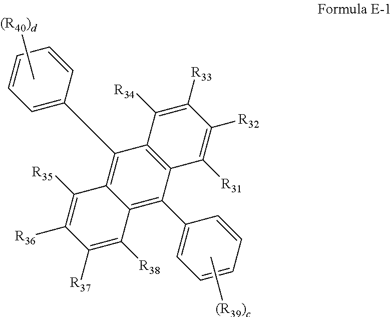

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In some embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer from 0 to 5.

Formula E-1 may be represented by one selected from among Compound E1 to Compound E19:

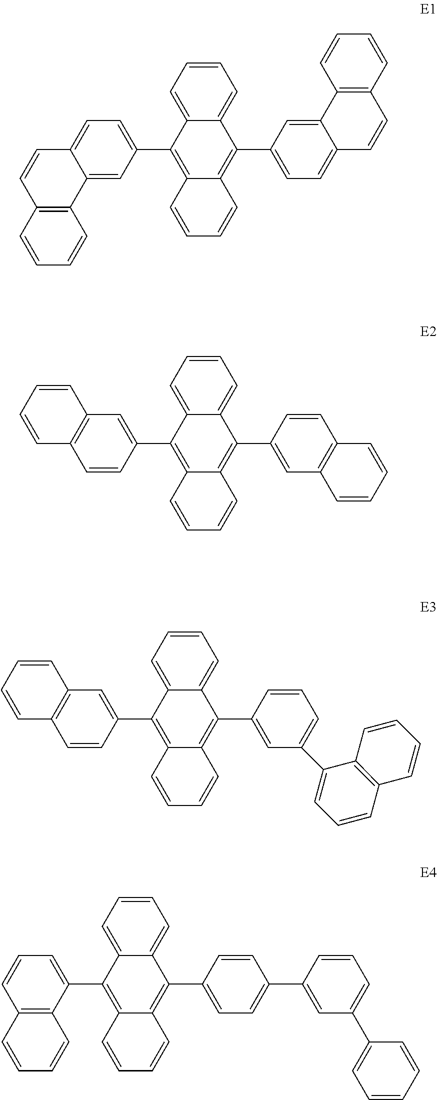

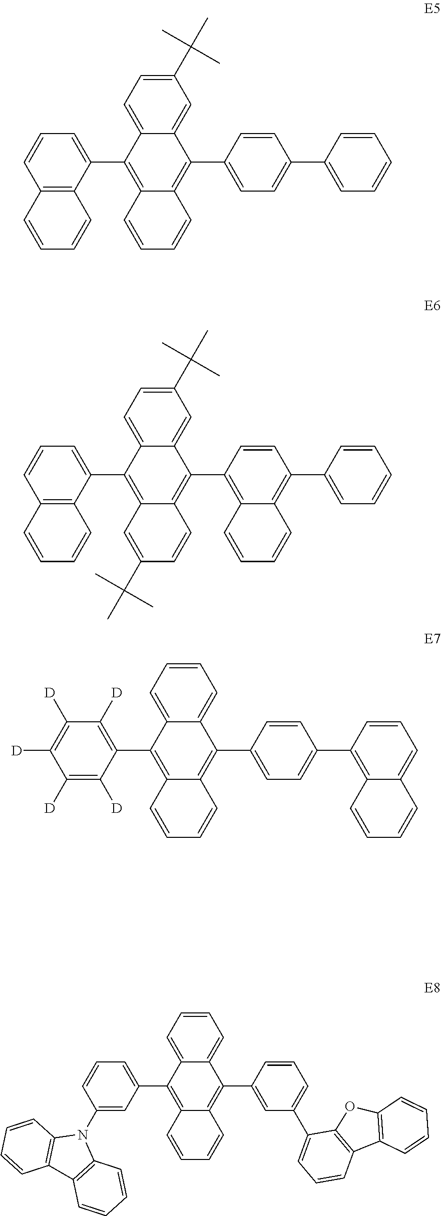

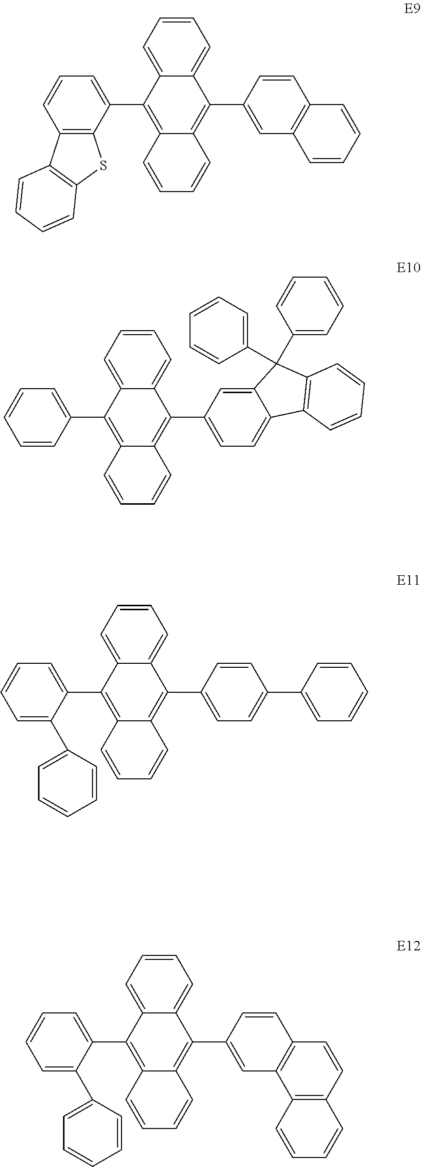

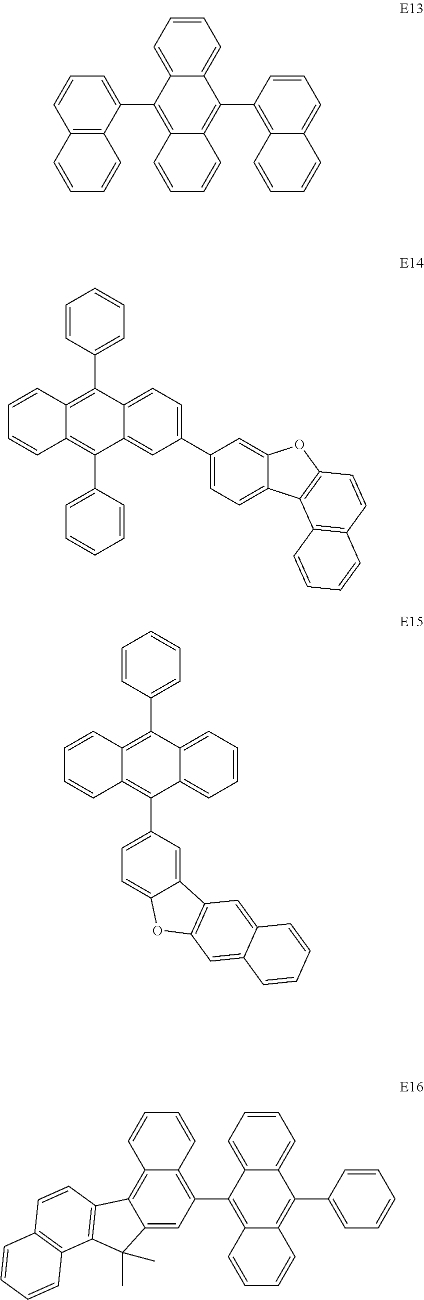

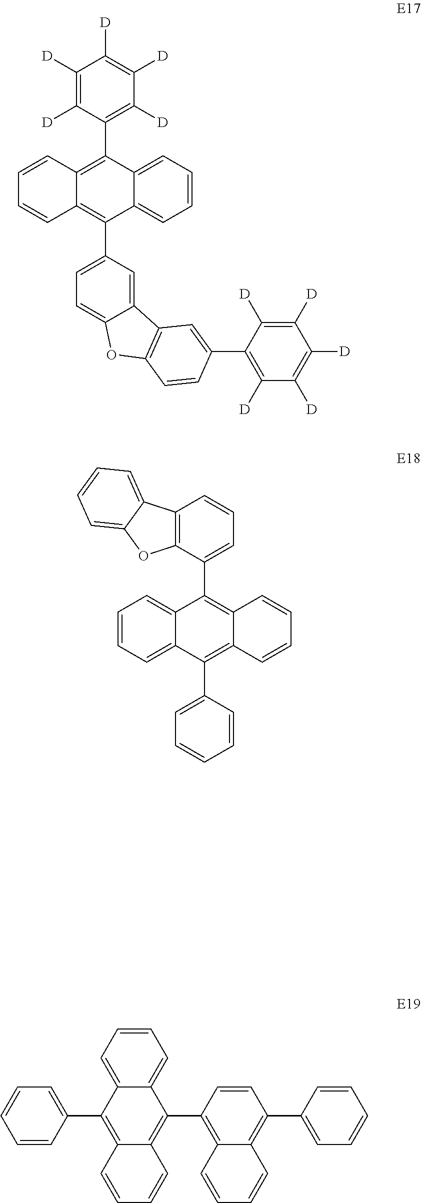

In an embodiment, the emission layer EML may include a compound represented by Formula E-2a or Formula E-2b. The compound represented by Formula E-2a or Formula E-2b may be used as a phosphorescent host material.

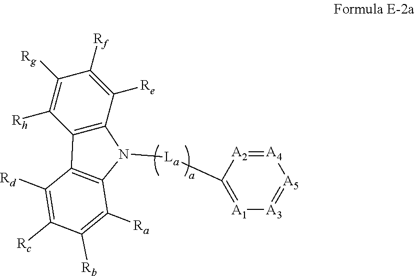

In Formula E-2a, a may be an integer from 0 to 10, La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or more, a plurality of Lₐs may each independently be a substituted or unsubstituted arylene group having 6 to 30 ring-forming carbon atoms, or a substituted or unsubstituted heteroarylene group having 2 to 30 ring-forming carbon atoms.

In addition, in Formula E-2a, A₁ to A₅ may each independently be N or CRᵢ. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to Rᵢ may be bonded to an adjacent group to form a hydrocarbon ring or a heterocycle containing N, O, S, etc. as a ring-forming atom.

In some embodiments, in Formula E-2a, two or three selected from among A₁ to A₅ may be N, and the rest (i.e., any of A₁ to A₅ that is not N) may be CRᵢ.

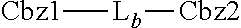

In Formula E-2b, Cbz1 and Cbz2 may each independently be an unsubstituted carbazole group, or a carbazole group substituted with an aryl group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, b may be an integer from 0 to 10, and when b is an integer of 2 or more, a plurality of L_{b}s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by one selected from among the compounds of Compound Group E-2. However, the compounds listed in Compound Group E-2 are merely examples, and the compounds represented by Formula E-2a or Formula E-2b are not limited to those represented in Compound Group E-2.

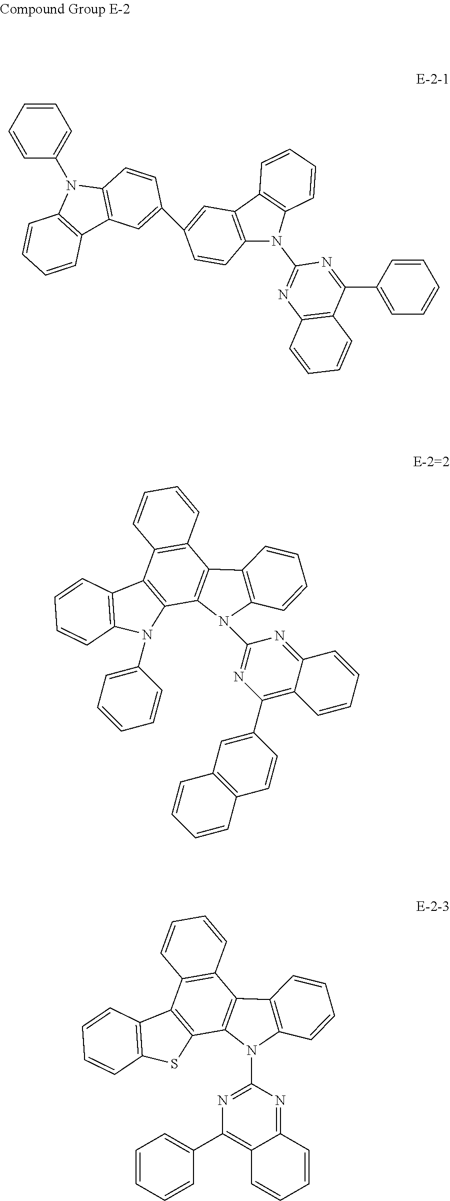 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

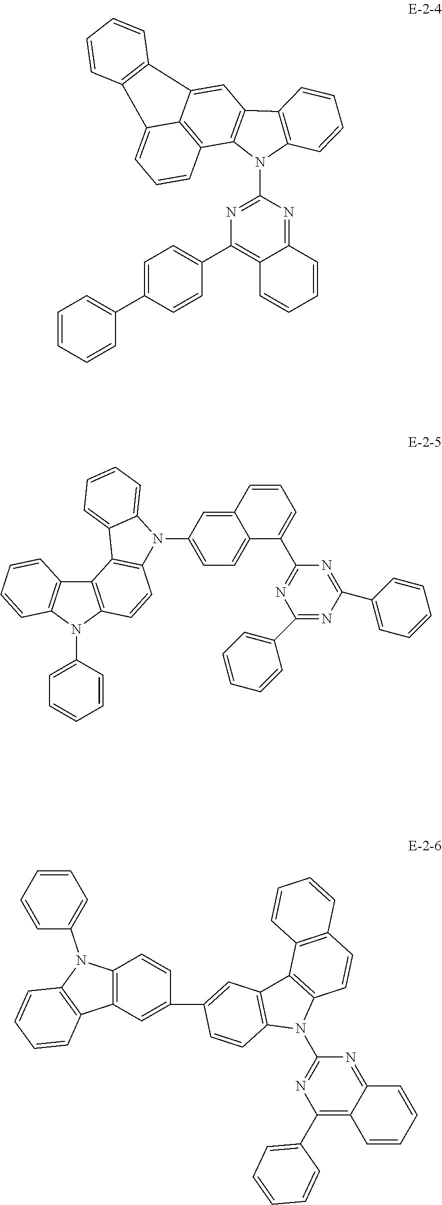 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

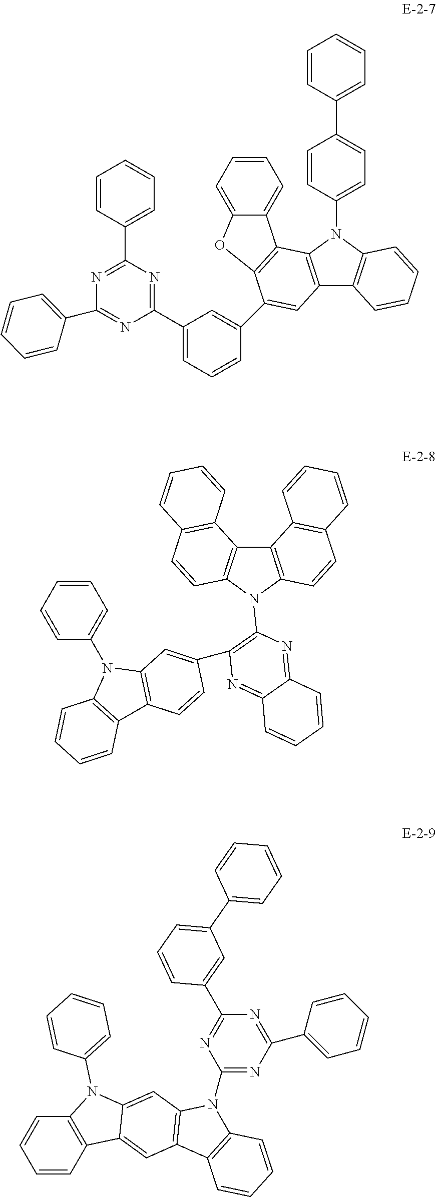 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

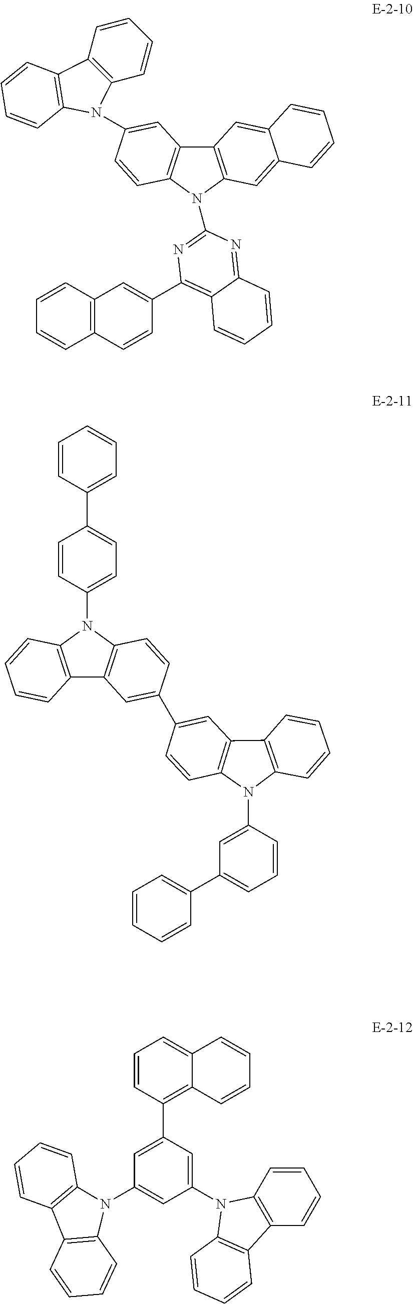 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

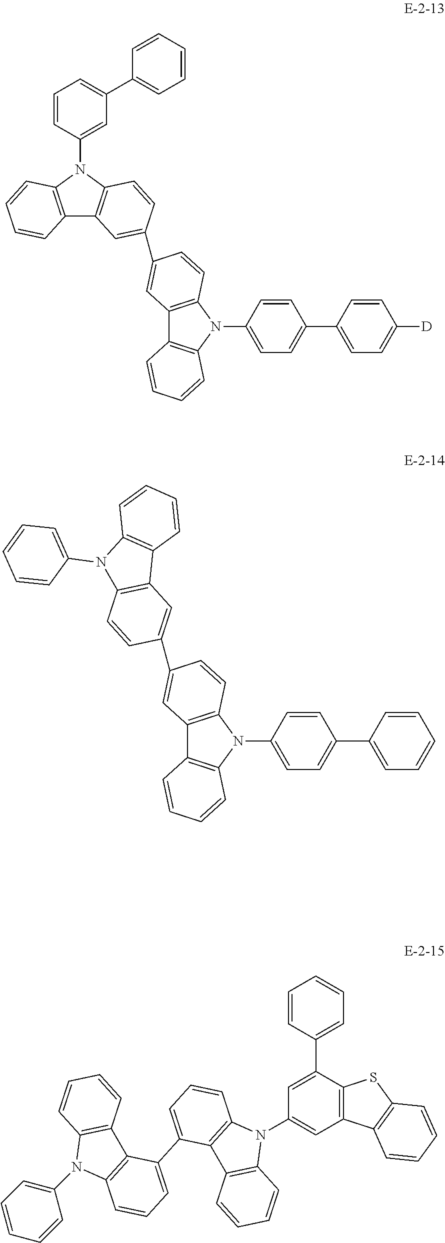 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

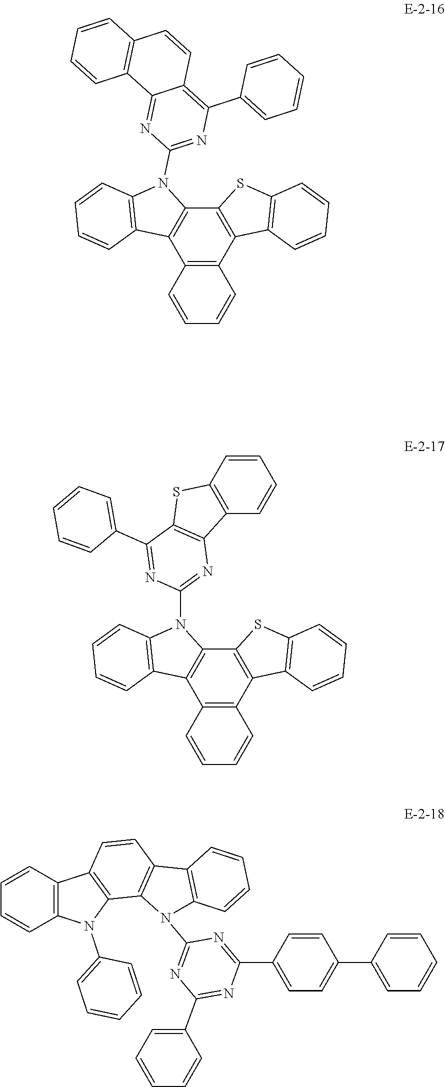 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

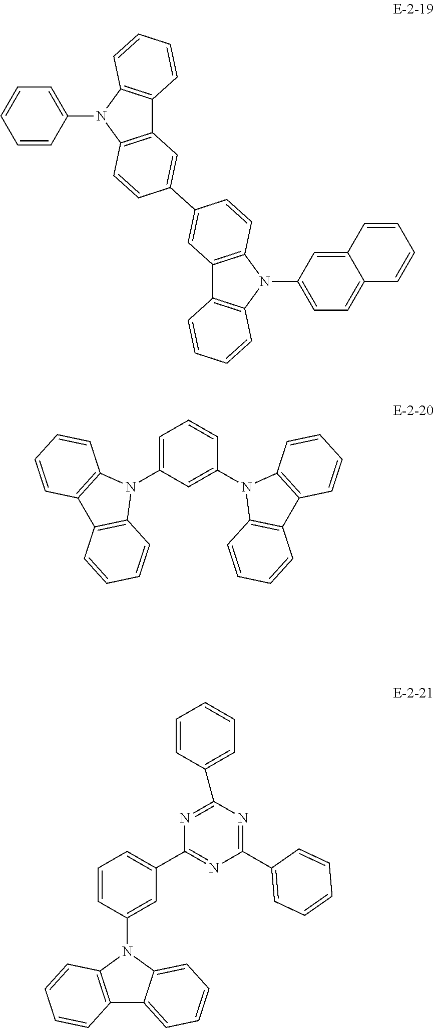 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

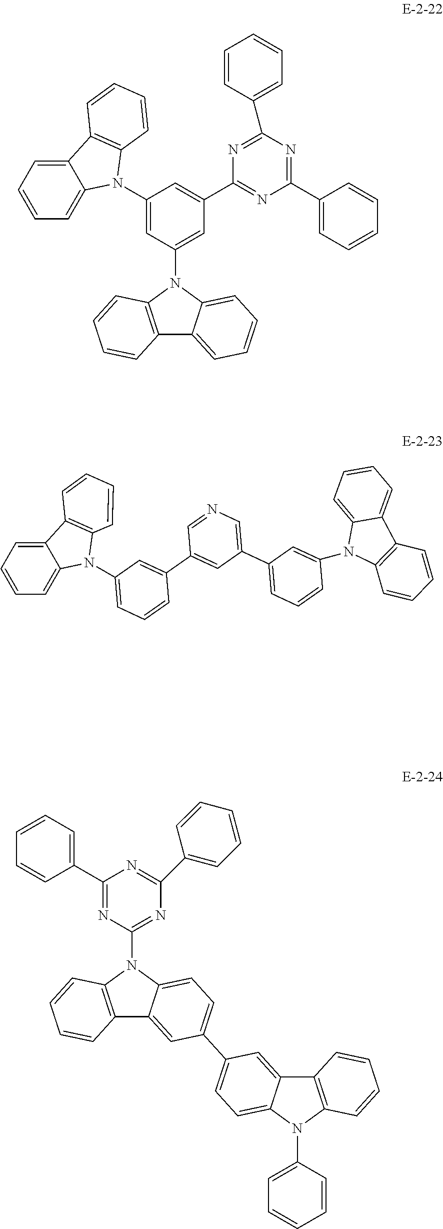 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyI)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiment of the present disclosure is not limited thereto, for example, tris(8-hydroxyquinolinato)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc.

The emission layer EML may include a compound represented by Formula M-a or Formula M-b. The compound represented by Formula M-a or Formula M-b may be used as a phosphorescence dopant material. In some embodiments, the compound represented by Formula M-a or Formula M-b may be used as an auxiliary dopant material.

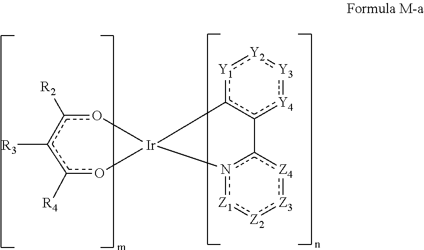

In Formula M-a, Y₁ to Y₄ and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m may be 0 or 1, and n may be 2 or 3. In Formula M-a, when m is 0, n is 3, and when m is 1, n is 2.

The compound represented by Formula M-a may be used as a phosphorescent dopant.

The compound represented by Formula M-a may be represented by one selected from among Compound M-a1 to Compound M-a25. However, Compounds M-a1 to M-a25 are merely examples, and the compound represented by Formula M-a is not limited to those represented by Compounds M-a1 to M-a25.

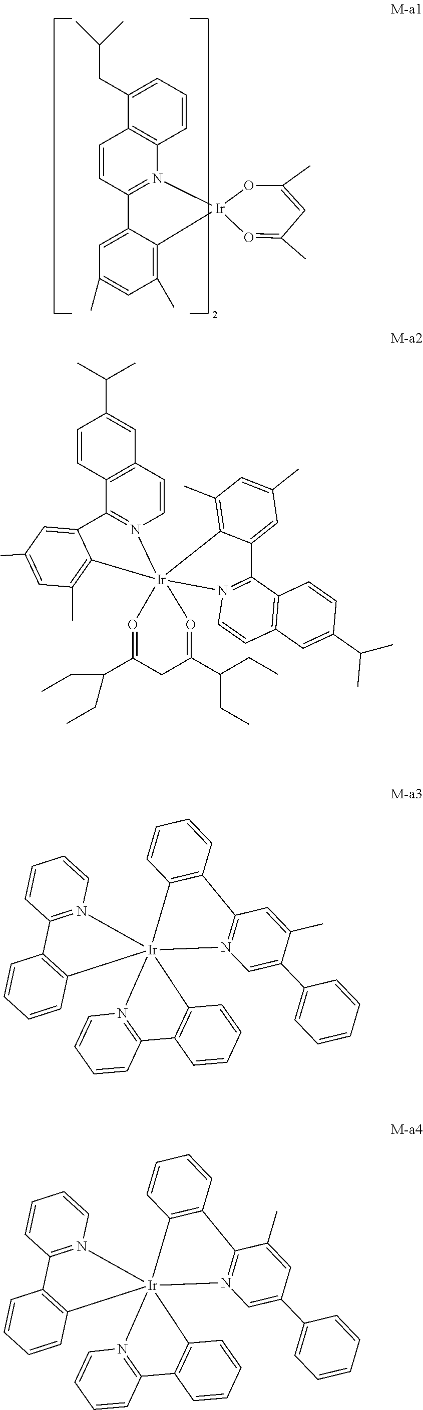

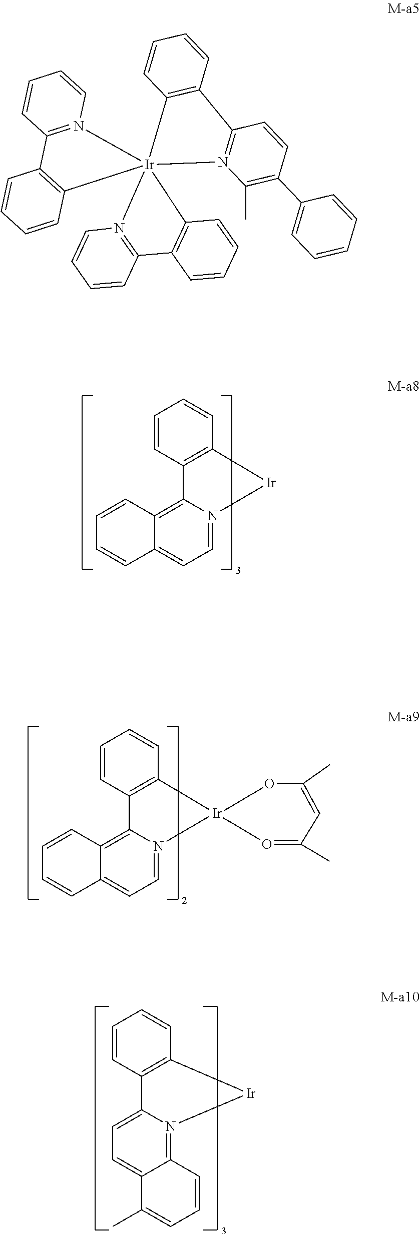

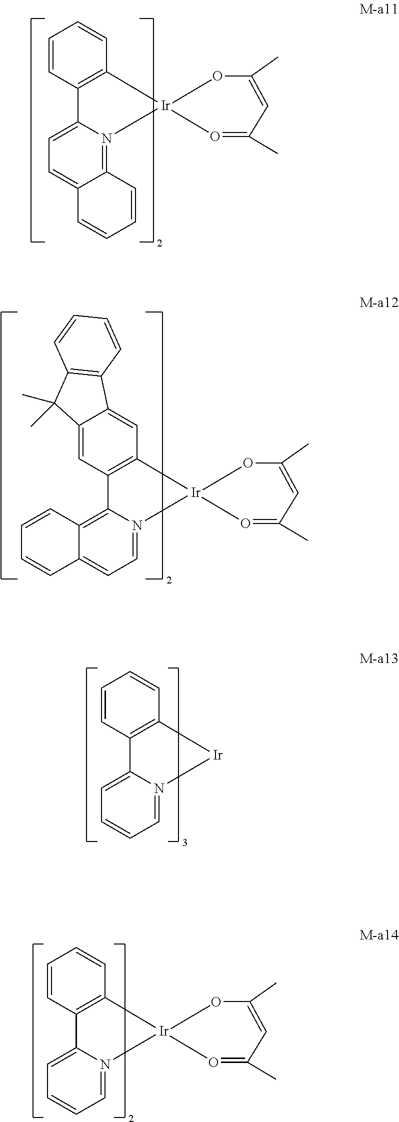

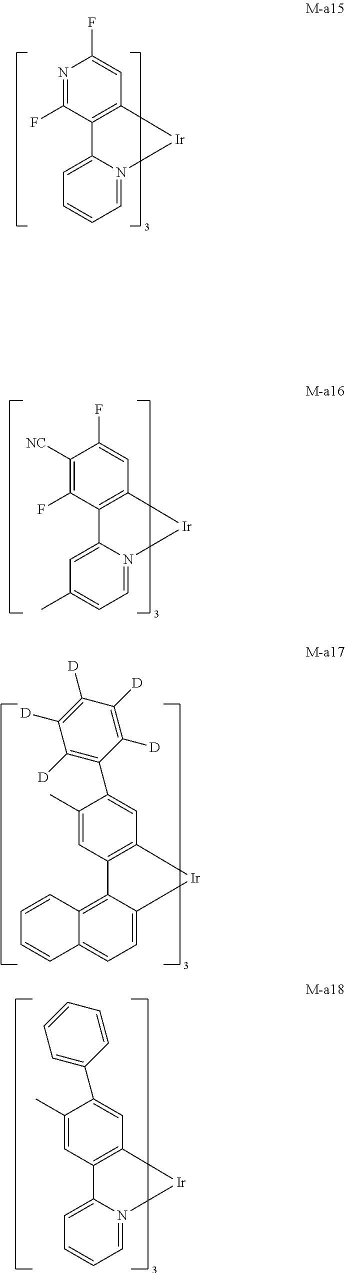

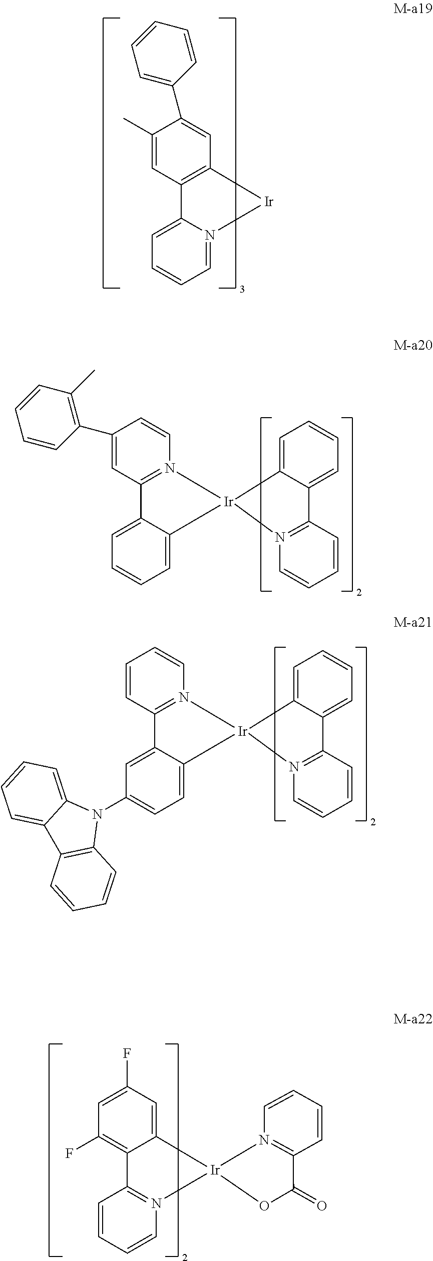

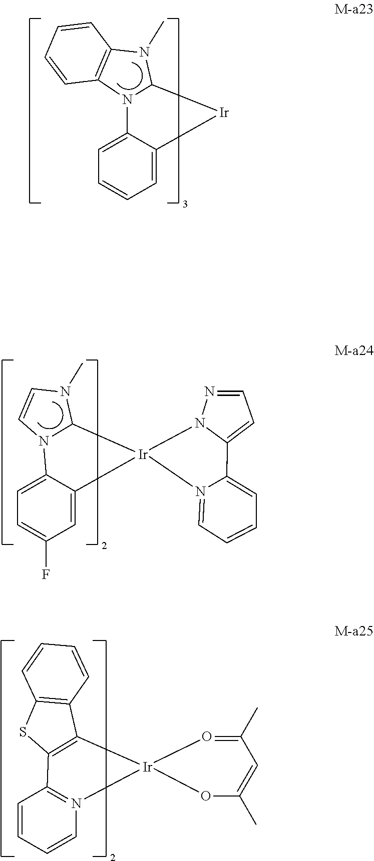

Compound M-a1 and Compound M-a2 may be used as a red dopant material, and Compound M-a3 to Compound M-a7 may be used as a green dopant material.

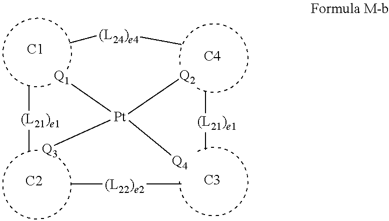

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ may each independently be a direct linkage,

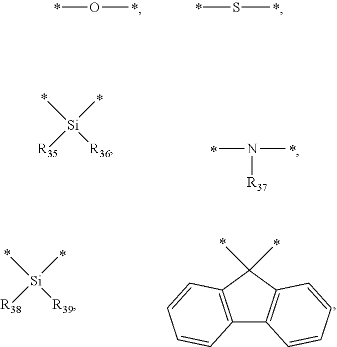

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and el to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and d1 to d4 may each independently be an integer from 0 to 4.

The compound represented by Formula M-b may be used as a blue phosphorescence dopant or a green phosphorescence dopant. In some embodiments, the compound represented by Formula M-b may be further included as an auxiliary dopant in the emission layer EML.

The compound represented by Formula M-b may be represented by one selected from among the compounds. However, the compounds are merely examples, and the compound represented by Formula M-b is not limited to those represented by the compounds.

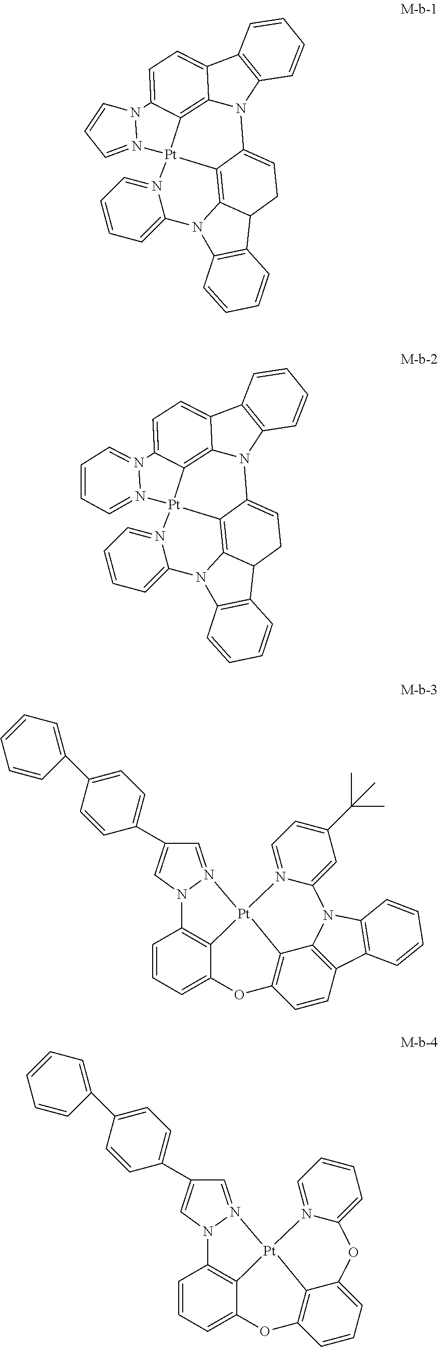

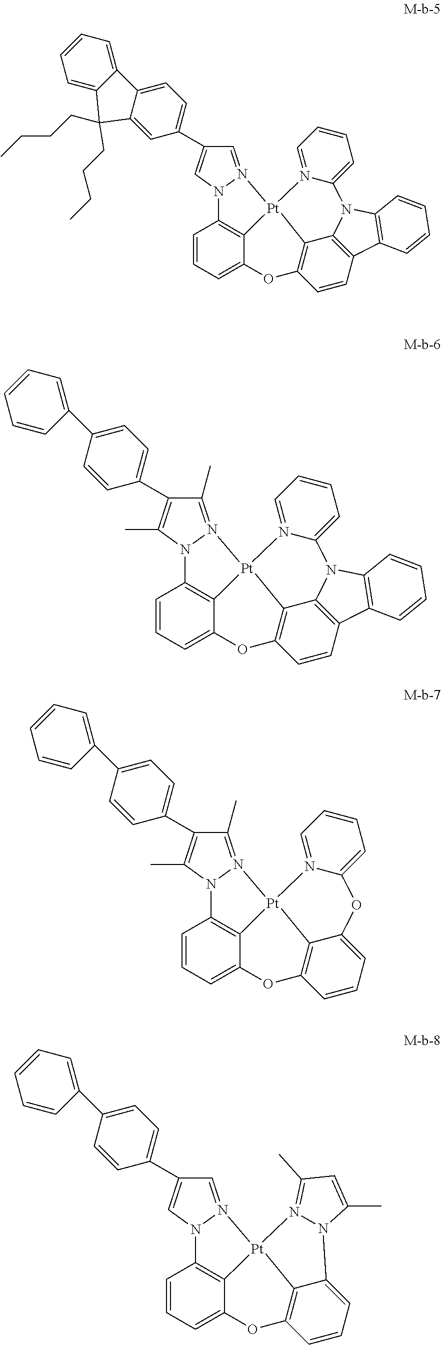

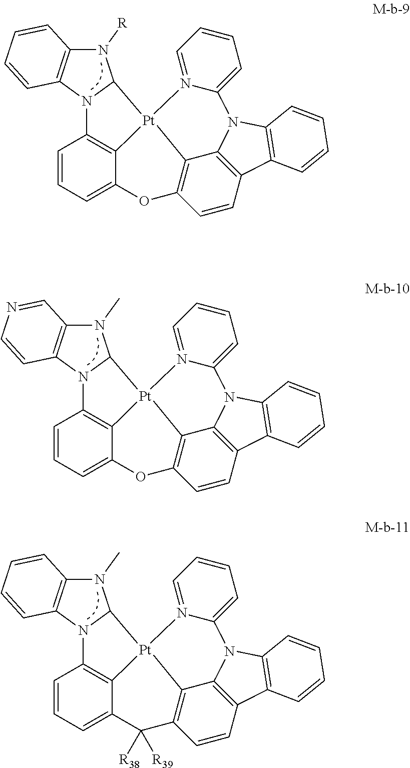

In the compounds, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may further include a compound represented by one selected from among Formula F-a to Formula F-c. The compound represented by Formula F-a or Formula F-c may be used as a fluorescence dopant material.

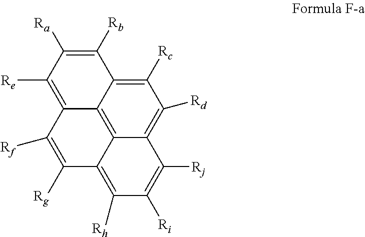

In Formula F-a, two selected from among Rₐ to Rⱼ may each independently be substituted with [symbol] NAr₁Ar₂ The others, which are not substituted with [symbol] NAr₁Ar₂ among Rₐ to Rⱼ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In [symbol] NAr₁Ar₂ Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one selected from Ar₁ and Ar₂ may be a heteroaryl group containing O or S as a ring-forming atom.

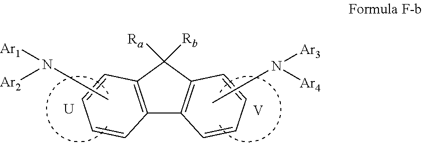

In Formula F-b, Rₐ and R_{b} may be each independently a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may each independently be 0 or 1. For example, in Formula F-b, when the number of U or V is 1, one ring constitutes a fused ring at a portion indicated by U or V, and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring in Formula F-b may be a cyclic compound having three rings. In some embodiments, when each number of U and V is 1, the fused ring having a fluorene core in Formula F-b may be a cyclic compound having five rings.

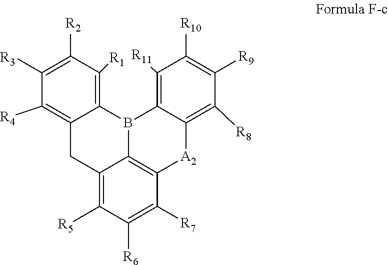

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a condensed ring. For example, when A₁ and A₂ may each independently be NRₘ, A₁ may be bonded to R₄ or R₅ to form a ring. In some embodiments, A₂ may be bonded to R₇ or R₈ to form a ring.

In an embodiment, the emission layer EML may include, as a known dopant material, a styryl derivative (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E)-4-(diphenylam ino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and a derivative thereof (e.g., 2,5,8,11-tetra-t-butylperylene (TBP)), pyrene and a derivative thereof (e.g., 1,1-dipyrene, 1,4-dipyrenylbenzene, 1,4-bis(N,N-diphenylamino)pyrene), etc.

In an embodiment, when a plurality of emission layers EML are included, at least one emission layer EML may include a suitable phosphorescence dopant material. For example, a metal complex containing iridium (Ir), platinum (Pt), osmium (Os), gold (Au), titanium (Ti), zirconium (Zr), hafnium (Hf), europium (Eu), terbium (Tb), or thulium (Tm) may be used as a phosphorescent dopant. For example, iridium(III) bis(4,6-difluorophenylpyridinato-N,C2′) picolinate (Flrpic), bis(2,4-difluorophenylpyridinato)-tetrakis(1-pyrazolyl)borate (Fir₆), or platinum octaethyl porphyrin (PtOEP) may be used as a phosphorescence dopant. However, the embodiment of the present disclosure is not limited thereto.

In some embodiments, the emission layer EML in an embodiment may include a hole transport host and an electron transport host. Also, the emission layer EML may include an auxiliary dopant and a light emitting dopant. In some embodiments, a phosphorescent dopant material or a thermally delayed fluorescent dopant material may be included as the auxiliary dopant. For example, the emission layer EML in an embodiment may include the hole transport host, the electron transport host, the auxiliary dopant, and the light emitting dopant.

In addition, in the emission layer EML, an exciplex may be formed by the hole transport host and the electron transport host. In this embodiment, a triplet energy of the exciplex formed by the hole transport host and the electron transport host may correspond to T1 that is a gap between a lowest unoccupied molecular orbital (LUMO) energy level of the electron transport host and a highest occupied molecular orbital (HOMO) energy level of the hole transport host.

In an embodiment, the triplet energy (T1) of the exciplex formed by the hole transport host and the electron transport host may be about 2.4 eV to about 3.0 eV. In some embodiments, the triplet energy of the exciplex may be a value smaller than an energy gap of each host material. Therefore, the exciplex may have a triplet energy of about 3.0 eV or less that is an energy gap between the hole transport host and the electron transport host.

In some embodiments, at least one emission layer EML may include a quantum dot material. The core of the quantum dot may be selected from among a Group II-VI compound, a Group III-VI compound, a Group compound, a Group III-V compound, a Group III-II-V compound, a Group IV-VI compound, a Group IV element, a Group IV compound, and one or more combinations thereof.

The Group II-VI compound may be selected from the group including (e.g., consisting of) a binary compound selected from the group including (e.g., consisting of) CdSe, CdTe, CdS, ZnS, ZnSe, ZnTe, ZnO, HgS, HgSe, HgTe, MgSe, MgS, and one or more mixtures thereof, a ternary compound selected from the group including (e.g., consisting of) CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and one or more mixtures thereof, and a quaternary compound selected from the group including (e.g., consisting of) HgZnTeS, CdZnSeS, CdZnSeTe, CdZnSTe, CdHgSeS, CdHgSeTe, CdHgSTe, HgZnSeS, HgZnSeTe, HgZnSTe, and one or more mixtures thereof.

The Group III-VI compound may include a binary compound such as In₂S₃ or In₂Se₃, a ternary compound such as InGaS₃ or InGaSe₃, or one or more combinations thereof.

The Group compound may be selected from a ternary compound selected from the group including (e.g., consisting of) AgInS, AgInS₂, CuInS, CuInS₂, AgGaS₂, CuGaS₂, CuGaO₂, AgGaO₂, AgAlO₂, and one or more mixtures thereof, or a quaternary compound such as AgInGaS₂ or CuInGaS₂.

The Group III-V compound may be selected from the group including (e.g., consisting of) a binary compound selected from the group including (e.g., consisting of) GaN, GaP, GaAs, GaSb, AIN, AIP, AlAs, AlSb, InN, InP, InAs, InSb, and one or more mixtures thereof, a ternary compound selected from the group including (e.g., consisting of) GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and one or more mixtures thereof, and a quaternary compound selected from the group including (e.g., consisting of) GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and one or more mixtures thereof. Meanwhile, the Group III-V compound may further include a Group II metal. For example, InZnP, etc. may be selected as a Group III-II-V compound.

The Group IV-VI compound may be selected from the group including (e.g., consisting of) a binary compound selected from the group including (e.g., consisting of) SnS, SnSe, SnTe, PbS, PbSe, PbTe, and one or more mixtures thereof, a ternary compound selected from the group including (e.g., consisting of) SnSeS, SnSeTe, SnSTe, PbSeS, PbSeTe, PbSTe, SnPbS, SnPbSe, SnPbTe, and one or more mixtures thereof, and a quaternary compound selected from the group consisting of SnPbSSe, SnPbSeTe, SnPbSTe, and one or more mixtures thereof. The Group IV element may be selected from the group including (e.g., consisting of) Si, Ge, and a mixture thereof. The Group IV compound may be a binary compound selected from the group including (e.g., consisting of) SiC, SiGe, and a mixture thereof.

In this case, a binary compound, a ternary compound, or a quaternary compound may be present in a particle with a substantially uniform concentration distribution, or may be present in the same particle with a partially different concentration distribution. In addition, a core/shell structure in which one quantum dot surrounds another quantum dot may also be possible. The core/shell structure may have a concentration gradient in which the concentration of elements present in the shell decreases toward the core.

In some embodiments, the quantum dot may have the above-described core/shell structure including a core containing nanocrystals and a shell surrounding the core. The shell of the quantum dot may serve as a protection layer to prevent (reduce) the chemical deformation of the core so as to maintain semiconductor properties, and/or a charging layer to impart electrophoresis properties to the quantum dot. The shell may be a single layer or a multilayer. An example of the shell of the quantum dot may include a metal or non-metal oxide, a semiconductor compound, or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but the present disclosure is not limited thereto.

In addition, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

The quantum dot may have a full width of half maximum (FWHM) of a light emission wavelength spectrum of about 45 nm or less, about 40 nm or less, and about 30 nm or less, and color purity or color reproducibility may be improved in the above range. In some embodiments, light emitted through such a quantum dot may be emitted in all directions, and thus a wide viewing angle may be improved (increased).

In some embodiments, although the form of a quantum dot is not limited as long as it is a form commonly used in the art, for example, a quantum dot in the form of substantially spherical, pyramidal, multi-arm, or cubic nanoparticles, nanotubes, nanowires, nanofibers, nanoparticles, etc. may be used.

The quantum dot may control the color of emitted light according to the particle size thereof. Accordingly, the quantum dot may have one or more suitable light emission colors such as blue, red, and green.

In each light emitting element ED of embodiments illustrated in FIGS. 3 to 6, the electron transport region ETR is provided on the emission layer EML. The electron transport region ETR may include at least one selected from the hole blocking layer HBL, the electron transport layer ETL, and the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL, an electron transport layer ETL/buffer layer/electron injection layer EIL are stacked in order from the emission layer EML, but the embodiment of the present disclosure is not limited thereto. The electron transport region ETR may have a thickness, for example, from about 1,000 Å to about 1,500 Å.

The electron transport region ETR may be formed by using one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1:

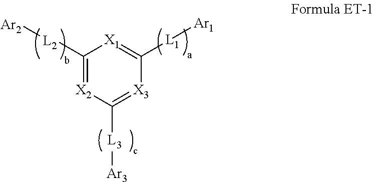

In Formula ET-1, at least one selected from among X₁, to X₃ is N, and the rest are (the X₁ to X₃ that are not N) CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer from 0 to 10. In Formula ET-1, L₁ to L3 may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c are an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]im idazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate) (Bebq2), 9,10-di(naphthalen-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), or one or more mixtures thereof.

The electron transport region ETR may include at least one selected from among Compound ET1 to Compound ET36:

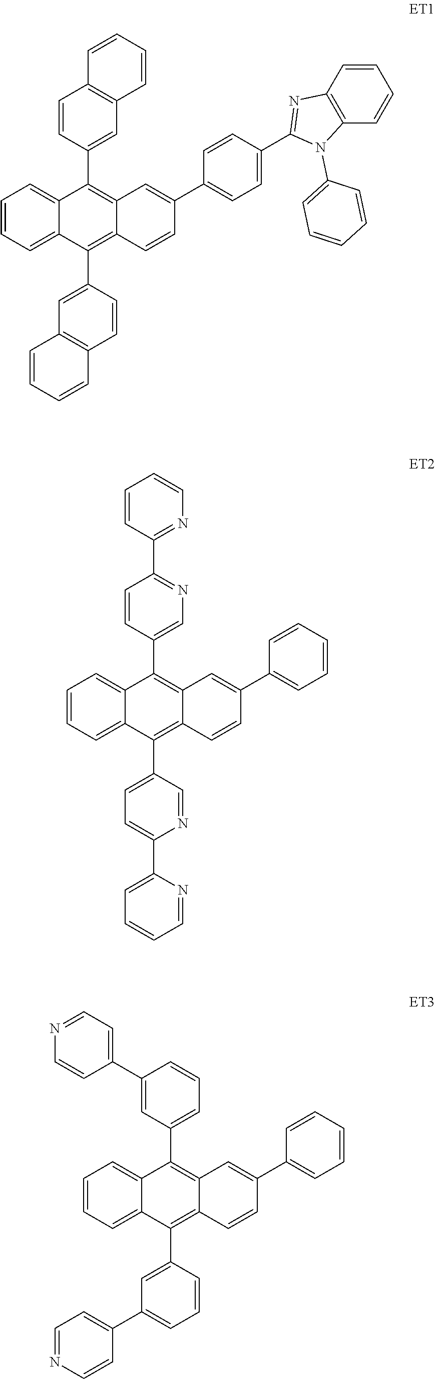 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

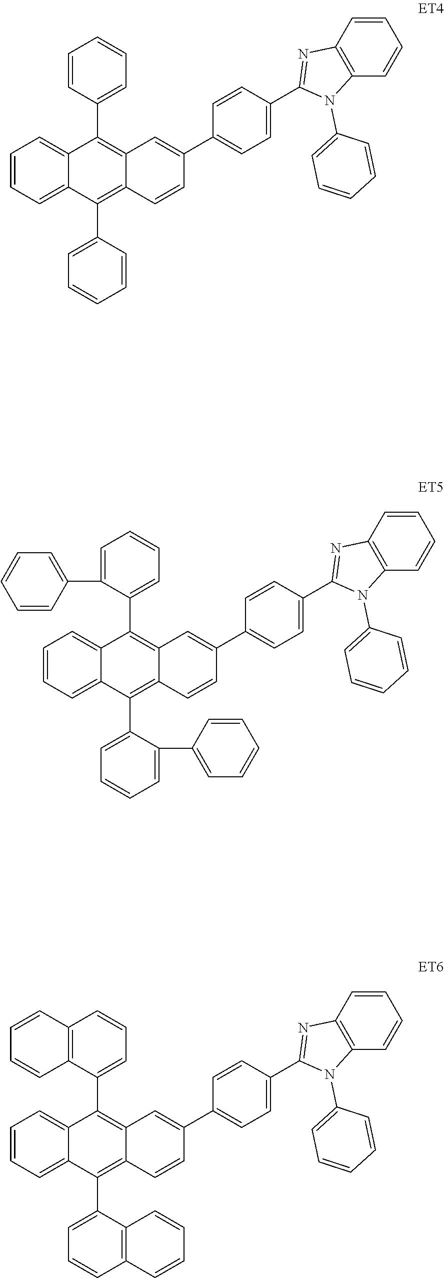 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

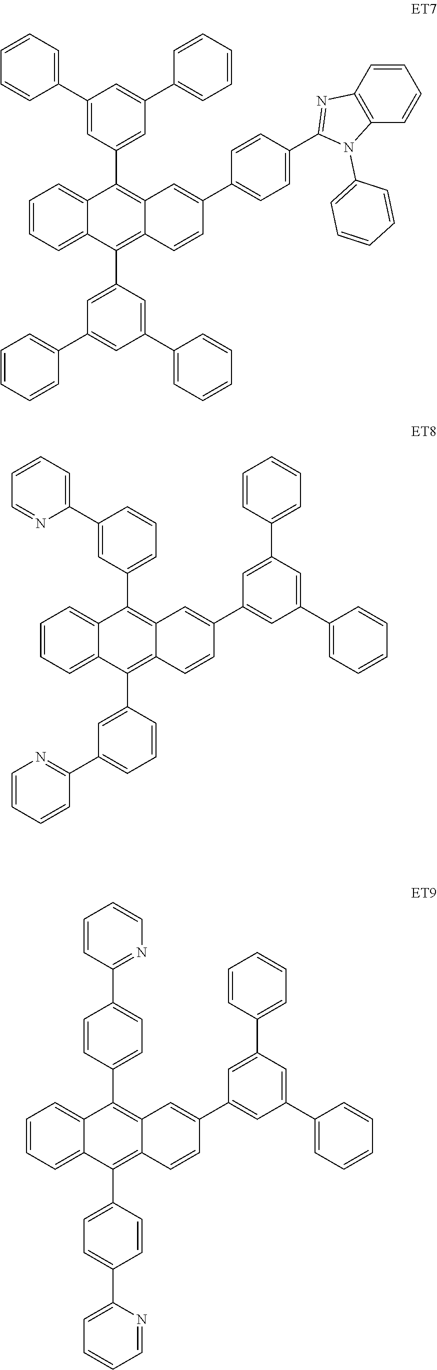 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

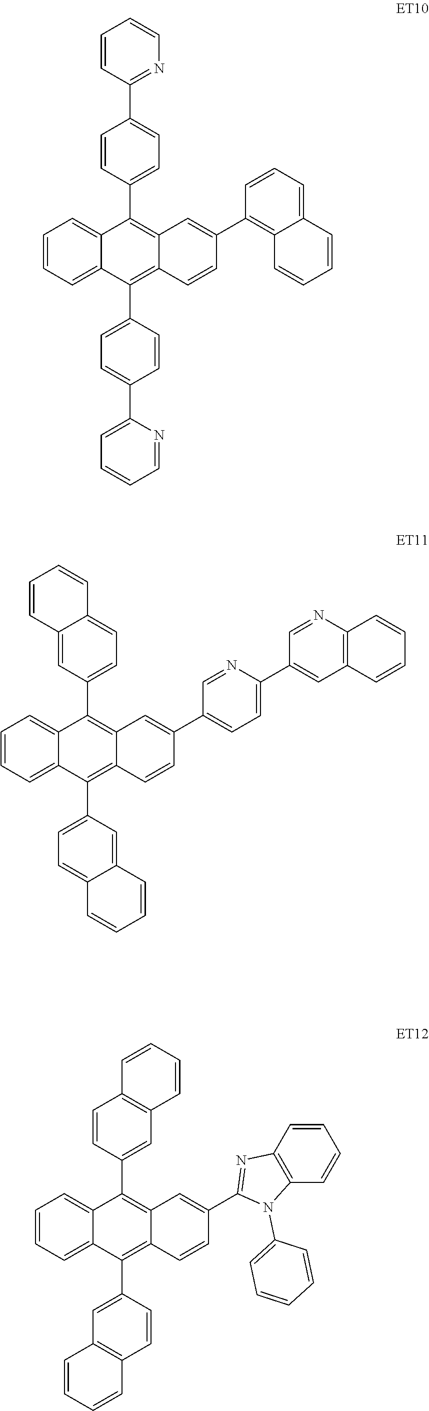 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

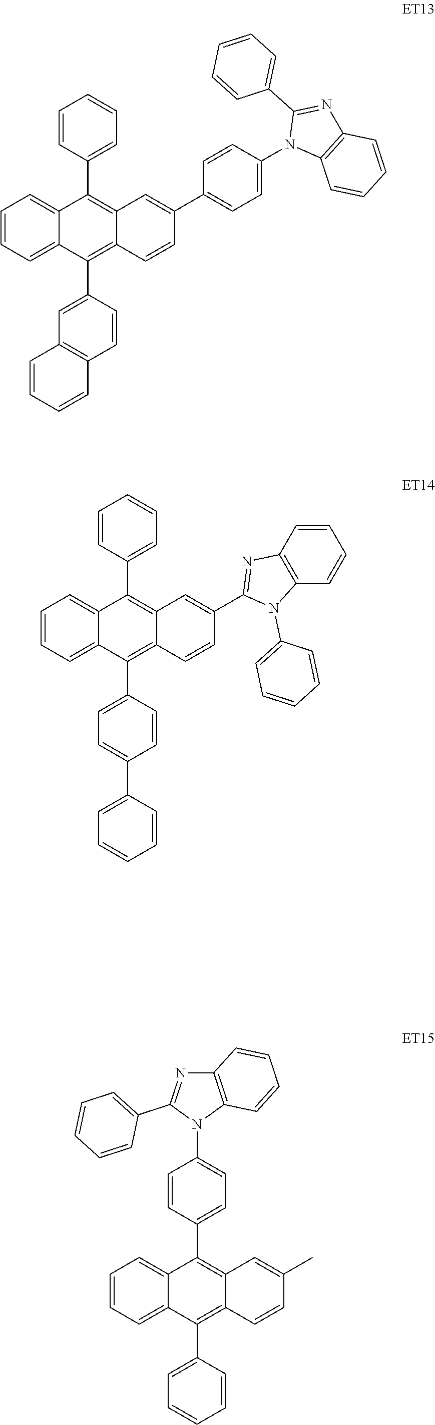 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

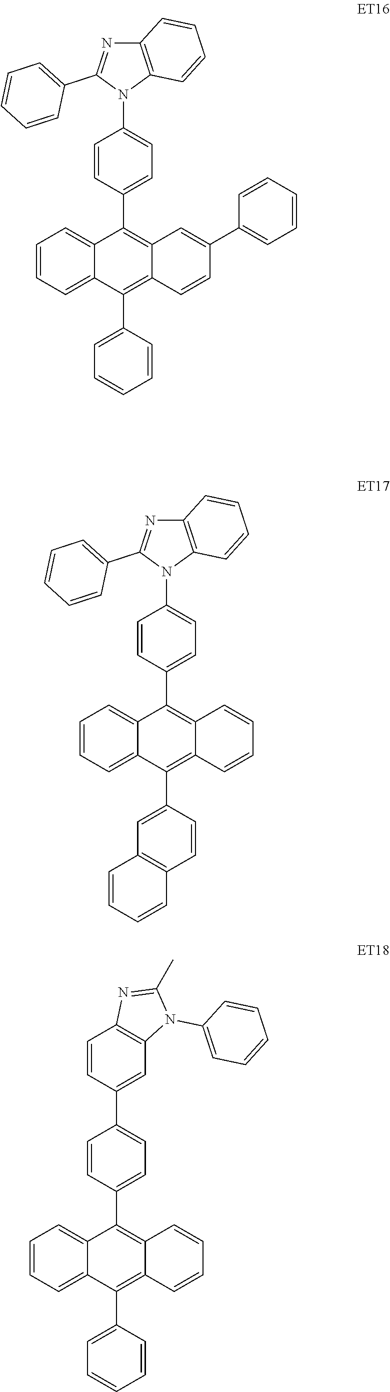 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

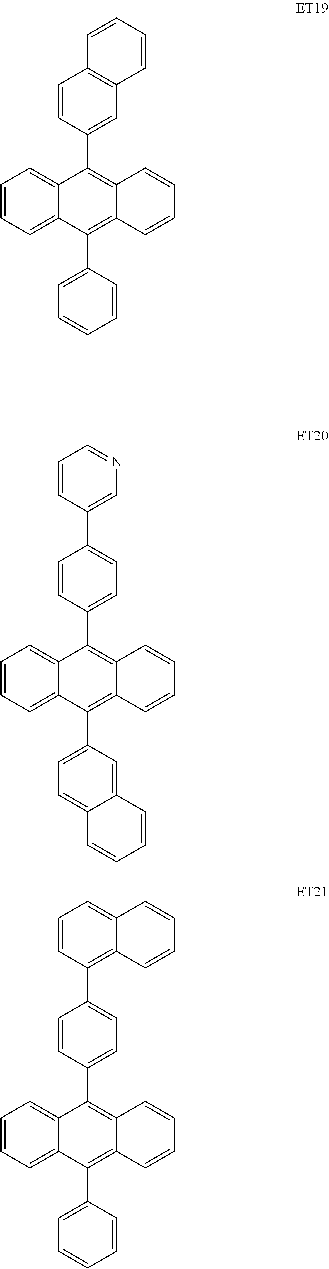

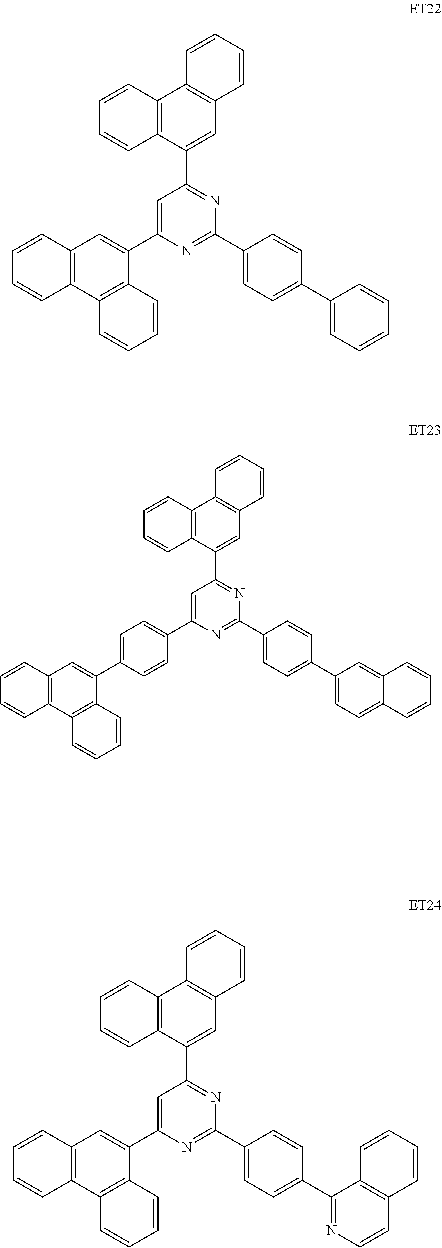 [SMILES: c1ccc(-c2ccc(-c3nc(-c4cc5ccccc5c5ccccc45)cc(-c4cc5ccccc5c5ccccc45)n3)cc2)cc1 ; c1ccc2cc(-c3ccc(-c4nc(-c5ccc(-c6cc7ccccc7c7ccccc67)cc5)cc(-c5cc6ccccc6c6ccccc56)n4)cc3)ccc2c1 ; c1ccc2c(-c3ccc(-c4nc(-c5cc6ccccc6c6ccccc56)cc(-c5cc6ccccc6c6ccccc56)n4)cc3)nccc2c1]

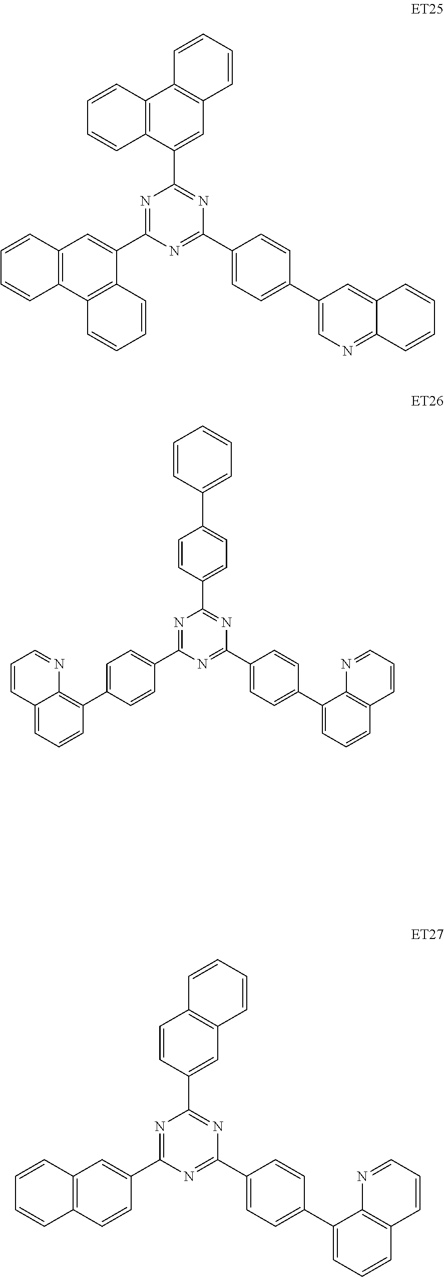 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

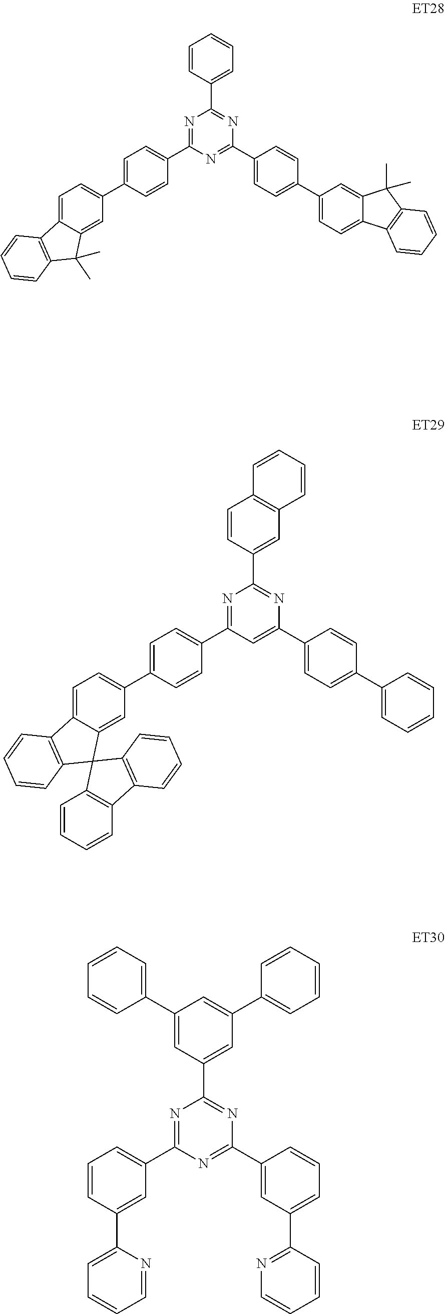 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc(-c5ccc6c(c5)C5(c7ccccc7-c7ccccc75)c5ccccc5-6)cc4)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

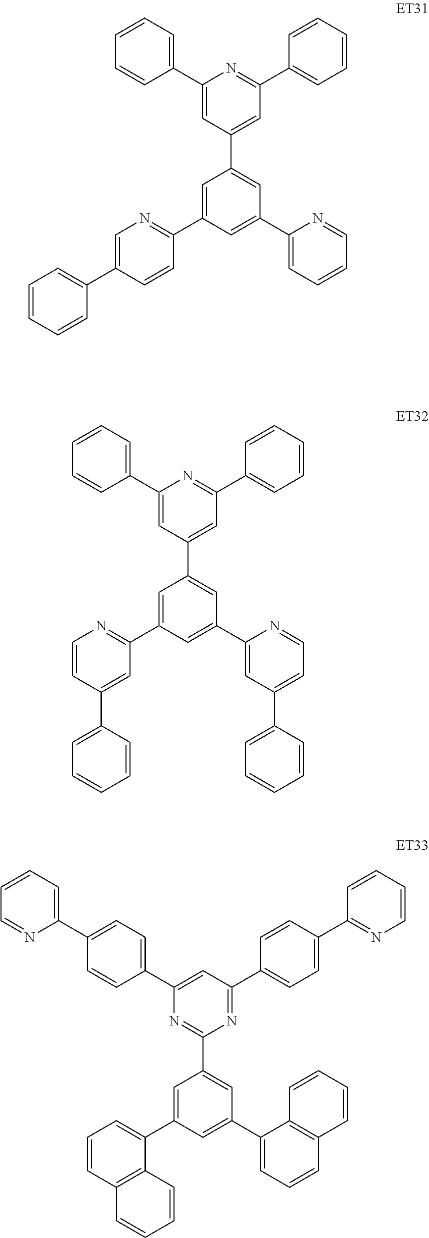 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

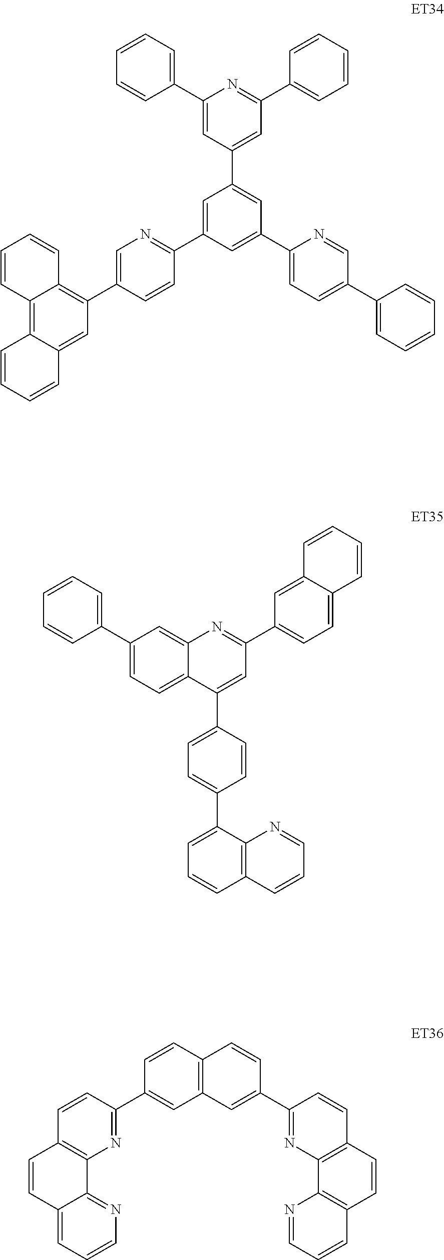 [SMILES: c1ccc(-c2ccc(-c3cc(-c4cc(-c5ccccc5)nc(-c5ccccc5)c4)cc(-c4ccc(-c5cc6ccccc6c6ccccc56)cn4)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport regions ETR may include a metal halide such as LiF, NaCl, CsF, RbCl, RbI, Cul, and/or KI, a lanthanide metal such as Yb, and a co-deposited material of the metal halide and the lanthanide metal.

For example, the electron transport region ETR may include KI:Yb, RbI:Yb, LiF:Yb, etc. as a co-deposited material. In some embodiments, the electron transport region ETR may be formed using a metal oxide such as Li₂O or BaO, or 8-hydroxyl-lithium quinolate (Liq), etc., but the embodiment of the present disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, or a metal stearate.

The electron transport region ETR may further include at least one selected from 2,9-dimethyl-4,7-diphenyl-1,10-phenanthroline (BCP), diphenyl(4-(triphenylsilyl)phenyl)phosphine oxide (TSPO1), and 4,7-diphenyl-1,10-phenanthroline (Bphen) in addition to the above-described materials, but the embodiment of the present disclosure is not limited thereto.

The electron transport region ETR may include the above-described compounds of the hole transport region in at least one selected from the electron injection layer EIL, the electron transport layer ETL, and the hole blocking layer HBL.

When the electron transport region ETR includes the electron transport layer ETL, the electron transport layer ETL may have a thickness of about 100 Å to about 1,000 Å, for example, about 150 Å to about 500 A. If the thickness of the electron transport layer ETL satisfies the aforementioned range, satisfactory (suitable) electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the above-described range, satisfactory (suitable) electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the present disclosure is not limited thereto. For example, when the first electrode EL1 is an anode, the second electrode EL2 may be a cathode, and when the first electrode EL1 is a cathode, the second electrode EL2 may be an anode. The second electrode may include at least one selected from among Ag, Mg, Cu, Al, Pt, Pd, Au, Ni, Nd, Ir, Cr, Li, Ca, LiF, Mo, Ti, W, In, Sn, Zn, compounds comprising one or more of the foregoing elements, combinations of two or more of the foregoing elements or compounds, mixtures of two or more of the foregoing elements or compounds, and oxides thereof.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, Yb, W, or a compound or mixture including these (e.g., AgMg, AgYb, or MgYb). Alternatively, the second electrode EL2 may have a multilayer structure including a reflective film or a transflective film formed of the above-described materials, and/or a transparent conductive film formed of ITO, IZO, ZnO, ITZO, etc. For example, the second electrode EL2 may include one or more of the above-described metal materials, combinations of at least two metal materials of the above-described metal materials, oxides of the above-described metal materials, and/or the like.

The second electrode EL2 may be connected with an auxiliary electrode. When the second electrode EL2 is connected with the auxiliary electrode, the resistance of the second electrode EL2 may be decreased.

In some embodiments, a capping layer CPL may further be disposed on the second electrode EL2 of the light emitting element ED. The capping layer CPL may include a multilayer or a single layer.

In an embodiment, the capping layer CPL may be an organic layer or an inorganic layer. For example, when the capping layer CPL contains an inorganic material, the inorganic material may include an alkaline metal compound (for example, LiF), an alkaline earth metal compound (for example, MgF₂), SiON, SiNₓ, SiOy, etc.

For example, when the capping layer CPL contains an organic material, the organic material may include a-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol sol-9-yl)triphenylamine (TCTA), etc., or may include an epoxy resin, or an acrylate such as a methacrylate. However, the embodiment of the present disclosure is not limited thereto, and the capping layer CPL may include at least one selected from among Compounds P1 to P5:

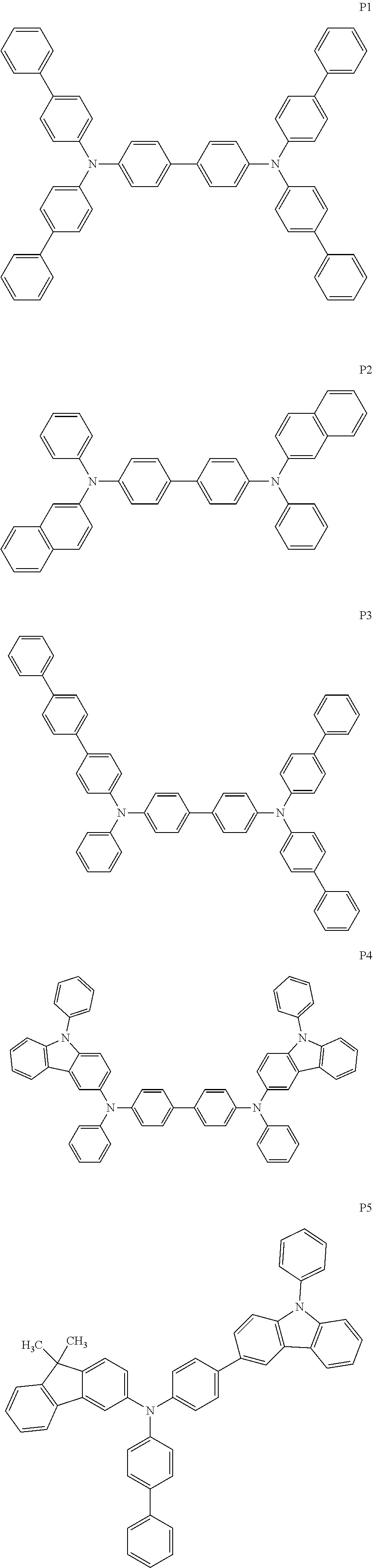 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be about 1.6 or more with respect to light in a wavelength range of about 550 nm to about 660 nm.

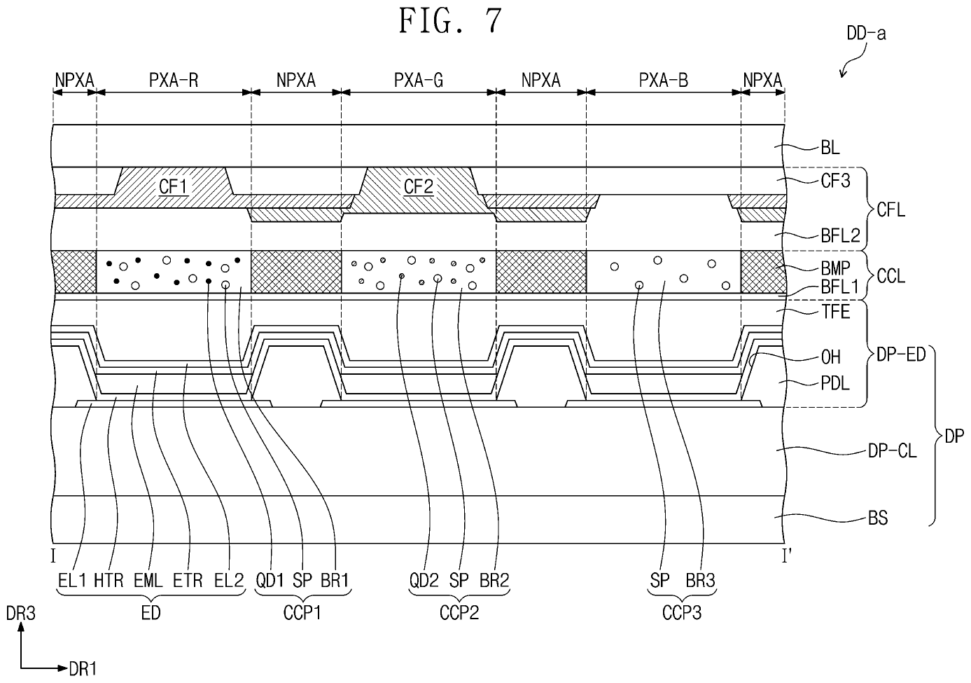

Each of FIGS. 7 to 10 is a cross-sectional view of a display device according to an embodiment of the present disclosure. Hereinafter, in describing the display devices of embodiments with reference to FIGS. 7 to 10, the duplicated features which have been described in FIGS. 1 to 6 may not be described again, but their differences will be primarily described.

Referring to FIG. 7, the display device DD-a according to an embodiment may include a display panel DP including a display element layer DP-ED, a light control layer CCL on the display panel DP, and a color filter layer CFL.

In an embodiment illustrated in FIG. 7, the display panel DP may include a base layer BS, a circuit layer DP-CL provided on the base layer BS, and the display element layer DP-ED, and the display element layer DP-ED may include a light emitting element ED.

The light emitting element ED may include a first electrode EIL1 , a hole transport region HTR on the first electrode EL1, an emission layer EML disposed on the hole transport region HTR, an electron transport region ETR on the emission layer EML, and a second electrode EL2 on the electron transport region ETR. In some embodiments, the structures of the light emitting elements of FIGS. 3 to 6 as described above may be equally applied to the structure of the light emitting element ED illustrated in FIG. 7.

The hole transport region HTR of the light emitting element ED included in the display device DD-a according to an embodiment may include the above-described amine compound.

Referring to FIG. 7, the emission layer EML may be disposed in an opening OH defined in a pixel defining film PDL. For example, the emission layer EML which is divided by the pixel defining film PDL and provided corresponding to each light emitting regions PXA-R, PXA-G, and PXA-B may emit light substantially in the same wavelength range. In the display device DD of an embodiment, the emission layer EML may emit blue light. In some embodiments, the emission layer EML may be provided as a common layer in the entire light emitting regions PXA-R, PXA-G, and PXA-B.

The light control layer CCL may be disposed on the display panel DP. The light control layer CCL may include a light conversion body. The light conversion body may be a quantum dot, a phosphor, and/or the like. The light conversion body may emit provided light by converting the wavelength thereof. For example, the light control layer CCL may be a layer containing the quantum dot or a layer containing the phosphor.

The light control layer CCL may include a plurality of light control parts CCP1, CCP2 and CCP3. The light control parts CCP1, CCP2, and CCP3 may be spaced apart (separated) from each other.

Referring to FIG. 7, divided patterns BMP may be disposed between the light control parts CCP1, CCP2 and CCP3 which are spaced apart from each other, but the embodiment of the present disclosure is not limited thereto. FIG. 7 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same as described above may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but may include the scatterer SP.

The scatterer SP may be inorganic particles. For example, the scatterer SP may include at least one of TiO₂, ZnO, Al₂O₃, SiO₂, or hollow sphere silica. The scatterer SP may include one selected from among TiO₂, ZnO, Al₂O₃, SiO₂, and hollow sphere silica, or may be a mixture or mixtures of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may be disposed on the light control parts CCP1, CCP2, and CCP3 to block or reduce the light control parts CCP1, CCP2 and CCP3 from being exposed to moisture/oxygen. In some embodiments, the barrier layer BFL1 may cover the light control parts CCP1, CCP2, and CCP3. In some embodiments, the barrier layer BFL2 may be provided between the color filter layer CFL and the light control parts CCP1, CCP2, and CCP3.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In some embodiments, the barrier layers BFL1 and BFL2 may further include an organic film. The barrier layers BFL1 and BFL2 may be formed of a single layer or a plurality of layers.

In the display device DD-a of an embodiment, the color filter layer CFL may be disposed on the light control layer CCL. For example, the color filter layer CFL may be directly disposed on the light control layer CCL. In this embodiment, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and/or a pigment or dye. The first filter CF1 may include a red pigment or dye, the second filter CF2 may include a green pigment or dye, and the third filter CF3 may include a blue pigment or dye. In some embodiments, the embodiment of the present disclosure is not limited thereto, and the third filter CF3 may not include a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

Furthermore, in an embodiment, the first filter CF1 and the second filter CF2 may be a yellow filter. The first filter CF1 and the second filter CF2 may not be separated but be provided as one filter. The first to third filters CF1, CF2, and CF3 may be disposed corresponding to the red light emitting region PXA-R, the green light emitting region PXA-G, and the blue light emitting region PXA-B, respectively.

Meanwhile, although not shown, the color filter layer CFL may include a light shielding part. The color filter layer CFL may include a light shielding part disposed to overlap at the boundaries of neighboring filters CF1, CF2, and CF3. The light shielding part may be a black matrix. The light shielding part may include an organic light shielding material and/or an inorganic light shielding material containing a black pigment or dye. The light shielding part may separate boundaries between the adjacent filters CF1, CF2, and CF3. In addition, in an embodiment, the light shielding part may be formed of a blue filter.

A base substrate BL may be disposed on the color filter layer CFL. The base substrate BL may be a member which provides a base surface in which the color filter layer CFL, the light control layer CCL, and/or the like are disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, the base substrate BL may not be provided.

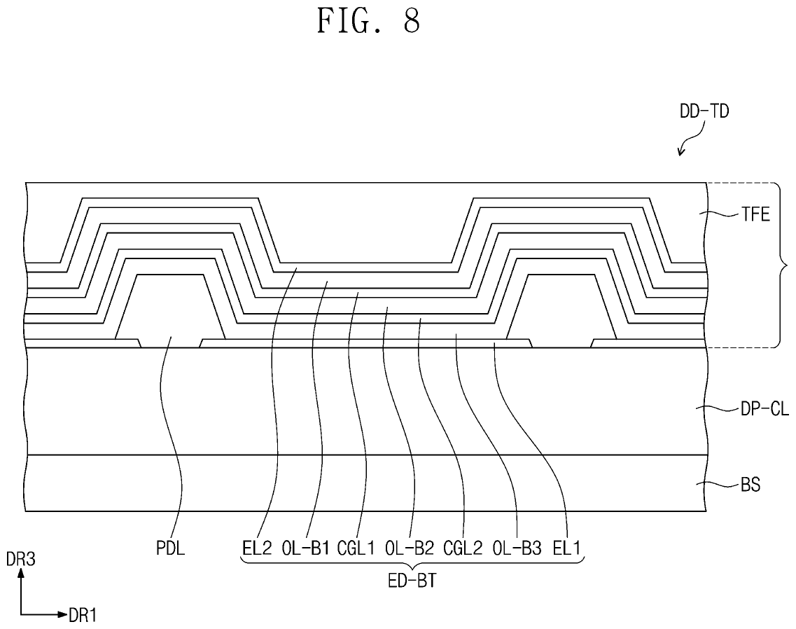

FIG. 8 is a cross-sectional view illustrating a portion of a display device according to an embodiment of the present disclosure. FIG. 8 illustrates a cross-sectional view of a part corresponding to the display panel DP of FIG. 7. In the display device DD-TD of an embodiment, the light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2 (e.g., stacked from the first electrode EL1). The light emitting structures OL-B1, OL-B2, and OL-B3 each may include an emission layer EML (FIG. 7) and may also include a hole transport region HTR and an electron transport region ETR disposed with the emission layer EML (FIG. 7) located therebetween.

For example, the light emitting element ED-BT included in the display device DD-TD of an embodiment may be a light emitting element having a tandem structure and including a plurality of emission layers.

In an embodiment illustrated in FIG. 8, all light beams respectively emitted from the light emitting structures OL-B1, OL-B2, and OL-B3 may be blue light. However, the embodiment of the present disclosure is not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may emit white light.

Charge generation layers CGL1 and CGL2 may be respectively disposed between two of the neighboring light emitting structures OL-B1, OL-B2, and OL-B3. The charge generation layers CGL1 and CGL2 may include a p-type or kind charge generation layer and/or an n-type or kind charge generation layer.

At least one of the light emitting structures OL-B1, OL-B2, or OL-B3 included in the display device DD-TD of an embodiment may contain the above-described amine compound.

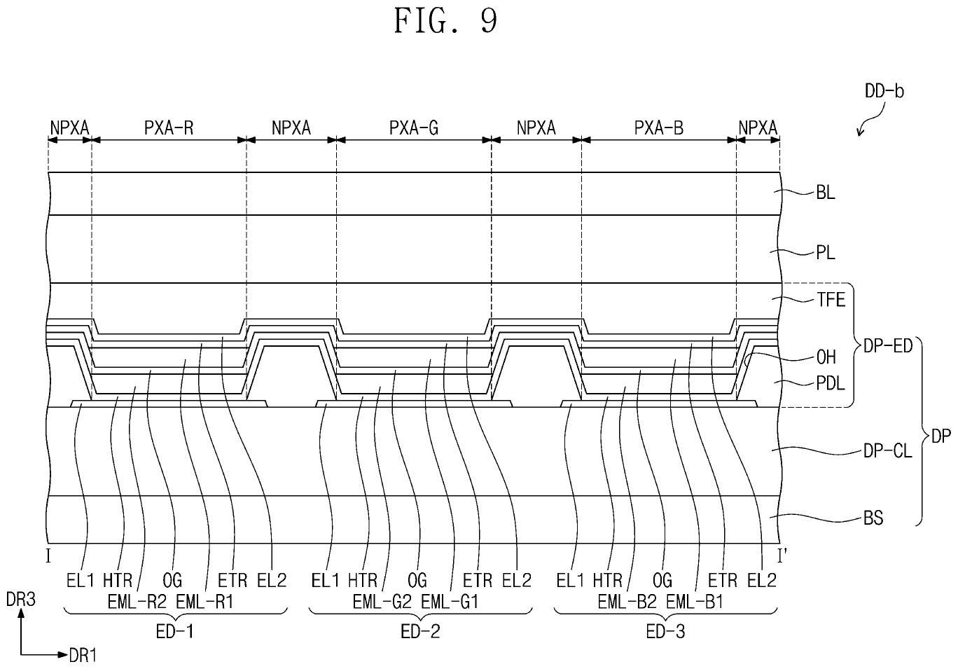

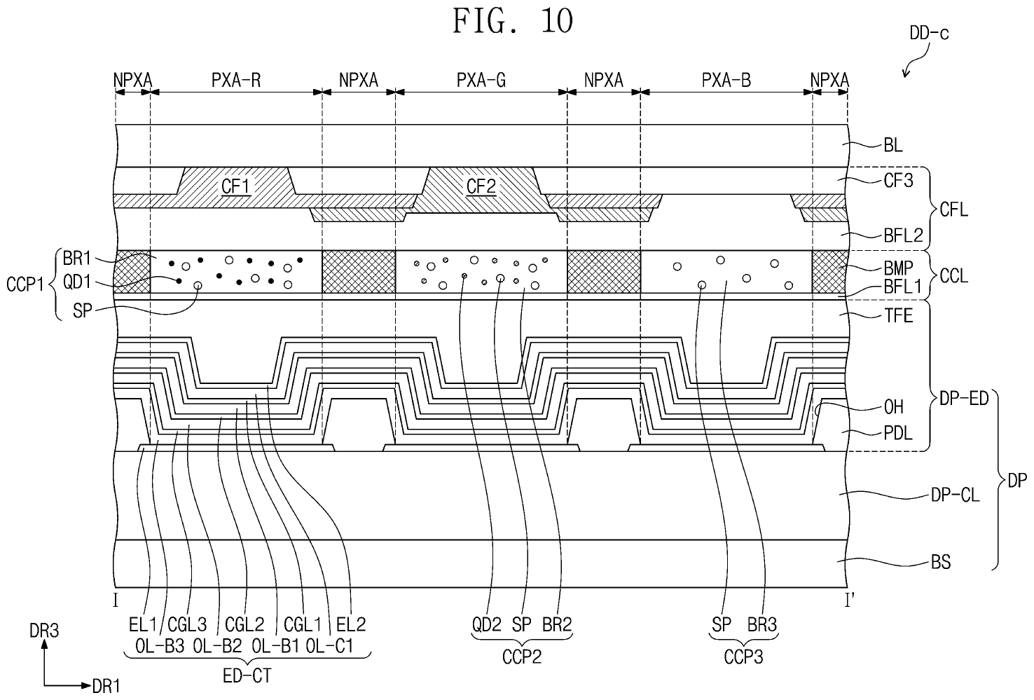

Referring to FIG. 9, the display device DD-b according to an embodiment may include light emitting elements ED-1, ED-2, and ED-3 in which two emission layers are stacked. Compared with the display device DD of an embodiment illustrated in FIG. 2, an embodiment illustrated in FIG. 9 has a difference in that the first to third light emitting elements ED-1, ED-2, and ED-3 each include two emission layers stacked in the thickness direction. In each of the first to third light emitting elements ED-1, ED-2, and ED-3, the two emission layers may emit light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. For example, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, the embodiment of the present disclosure is not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be disposed between the electron transport region ETR and the emission auxiliary part OG. The second red emission layer EML-R2, the second green emission layer EML-G2, and the second blue emission layer EML-B2 may be disposed between the emission auxiliary part OG and the hole transport region HTR.

For example, the first light emitting element ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked. The second light emitting element ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting element ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

In some embodiments, an optical auxiliary layer PL may be disposed on the display element layer DP-ED. The optical auxiliary layer PL may include a polarizing layer. The optical auxiliary layer PL may be disposed on the display panel DP and control reflected light in the display panel DP due to external light. Unlike the configuration illustrated, the optical auxiliary layer PL in the display device according to an embodiment may not be provided.

Unlike FIGS. 8 and 9, FIG. 10 illustrates that a display device DD-c includes four light emitting structures OL-B1, OL-B2, OL-B3, and OL-C1. Å light emitting element ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be disposed between the first to fourth light emitting structures OL-B1, OL-B2, OL-B3, and OL-C1. Among the four light emitting structures, the first to third light emitting structures OL-B1, OL-B2, and OL-B3 may emit blue light, and the fourth light emitting structure OL-C1 may emit green light. However, the embodiment of the present disclosure is not limited thereto, and the first to fourth light emitting structures OL-B1, OL-B2, OL-B3, and OL-C1 may emit light beams in different wavelength regions.

The charge generation layers CGL1, CGL2, and CGL3 disposed between adjacent light emitting structures OL-B1, OL-B2, OL-B3, and OL-C1 may include a p-type or kind charge generation layer and/or an n-type or kind charge generation layer.

At least one among the light emitting structures OL-B1, OL-B2, OL-B3, and OL-C1 included in the display device DD-c of an embodiment may include the above-described amine compound.

The light emitting element ED according to an embodiment of the present disclosure may include the above-described amine compound of an embodiment in at least one functional layer disposed between the first electrode ELI and the second electrode EL2, thereby exhibiting improved luminous efficiency and service life characteristics. The light emitting element ED according to an embodiment may include the above-described amine compound of an embodiment in at least one of the hole transport region HTR, the emission layer EML, or the electron transport region ETR disposed between the first electrode ELI and the second electrode EL2, or in a capping layer CPL.

For example, the amine compound according to an embodiment may be included in the hole transport region HTR of the light emitting element ED of an embodiment, and the light emitting element of an embodiment may exhibit excellent (suitable) luminous efficiency and long service life characteristics.

The above-described amine compound may improve the stability of radical or radical cation state, and may include the benzocarbazole moiety having an improved hole transport property due to the enhancement of the intermolecular π-π interaction, thereby contributing to reduced driving voltage and high efficiency of the light emitting element. In some embodiments, the amine compound may have an improvement in electron resistance and exciton resistance of the material by introducing, into a nitrogen atom part, at least one substituent selected from a naphthyl group, a phenanthryl group, a benzoheterole group, and a fluorenyl group. Accordingly, the light emitting element including the amine compound of an embodiment may have an improvement in efficiency and service life.

Hereinafter, with reference to Examples and Comparative Examples, an amine compound according to an embodiment of the present disclosure and a light emitting element of an embodiment of the present disclosure will be described in more detail. Examples described below are merely illustrations to assist the understanding of the present disclosure, and the scope of the present disclosure is not limited thereto.

### EXAMPLES

### 1. Synthesis of Amine Compound

First, a synthetic method of an amine compound according to the present embodiment will be described in more detail by illustrating the synthetic methods of Compounds A10, A110, A152, B11, B130, B163, B198, C1, C35, C47, C90, C175, C214, C293, C328, D16, and D60. Also, in the following descriptions, the synthetic method of the amine compound is provided as an example, but the synthetic method according to an embodiment of the present disclosure is not limited to the following Examples.

### (1) Synthesis of Compound A10

Amine Compound A10 according to an example may be synthesized by, for example, the steps shown in the following Reaction Schemes:

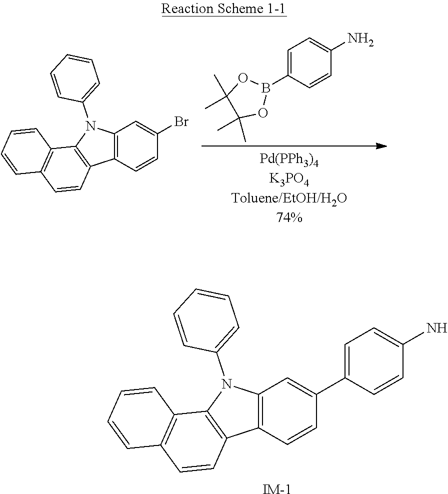

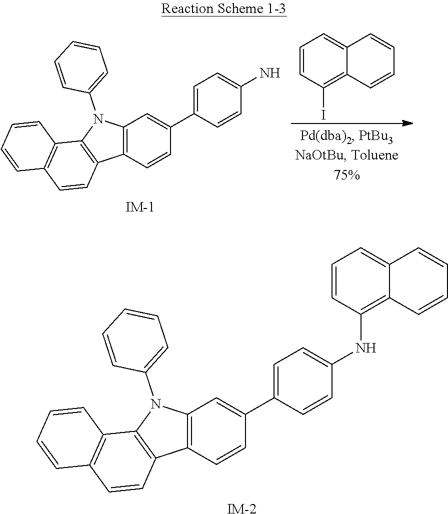

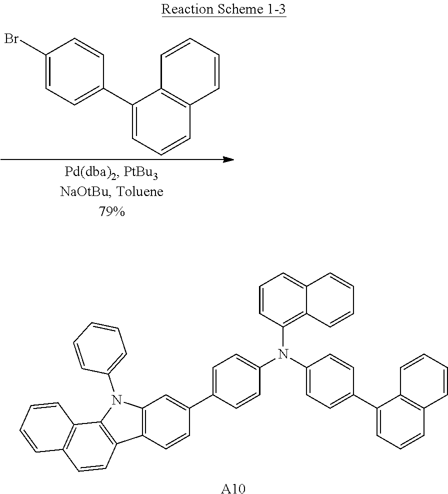 [SMILES: Brc1ccc(-c2cccc3ccccc23)cc1 ; c1ccc(-n2c3cc(-c4ccc(N(c5ccc(-c6cccc7ccccc67)cc5)c5cccc6ccccc56)cc4)ccc3c3ccc4ccccc4c32)cc1 ; "A10"]

### 1) Synthesis of Compound IM-1

In an argon (Ar) atmosphere, in a 2000 mL three-neck flask, 9-bromo-11-phenyl-11H-benzo[a]carbazole (50.00 g, 134.3 mmol), 4-(4,4,5,5-tetramethyl-1,3,2-dioxaborolan-2-yl)aniline (32.37 g, 1.1 equiv, 147.7 mmol), K₃PO₄ (85.53 g, 3.0 equiv, 402.9 mmol), Pd(PPh₃)₄ (7.76 g, 0.05 eq, 6.7 mmol), and a mixed solution of toluene/Et0H/H₂O (4/2/1) (940 mL) were sequentially added, and heated and stirred at about 80 ° C. After air-cooled to room temperature, the reaction solution was extracted with toluene to obtain organic layers. A water layer was removed, and the organic layers were washed with saturated saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-1 (38.21 g, yield 74%).

By measuring FAB-MS, a mass number of m/z=384 was observed by molecular ion peak, thereby identifying Compound IM-1.

### 2) Synthesis of Compound IM-2

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 1-iodonaphthalene (10.90 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-2 (14.94 g, yield 75%).

By measuring FAB-MS, a mass number of m/z=510 was observed by molecular ion peak, thereby identifying Compound IM-2.

### 3) Synthesis of Compound A10

In an Ar atmosphere, in a 300 mL three-neck flask, Compound IM-2 (10.00 g, 19.6 mmol), Pd(dba)₂ (0.34 g, 0.03 equiv, 0.6 mmol), NaO^{t}Bu (3.76 g, 2.0 equiv, 39.2 mmol), toluene (98 mL), 1-(4-bromophenyl)naphthalene (6.10 g, 1.1 equiv, 21.5 mmol), and PtBu₃ (0.40 g, 0.1 equiv, 2.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound A10 (11.03 g, yield 79%).

By measuring FAB-MS, a mass number of m/z=712 was observed by molecular ion peak, thereby identifying Compound A10.

### (2) Synthesis of Compound A110

Amine Compound A110 according to an example may be synthesized by, for example, the steps shown in Reaction Scheme 2:

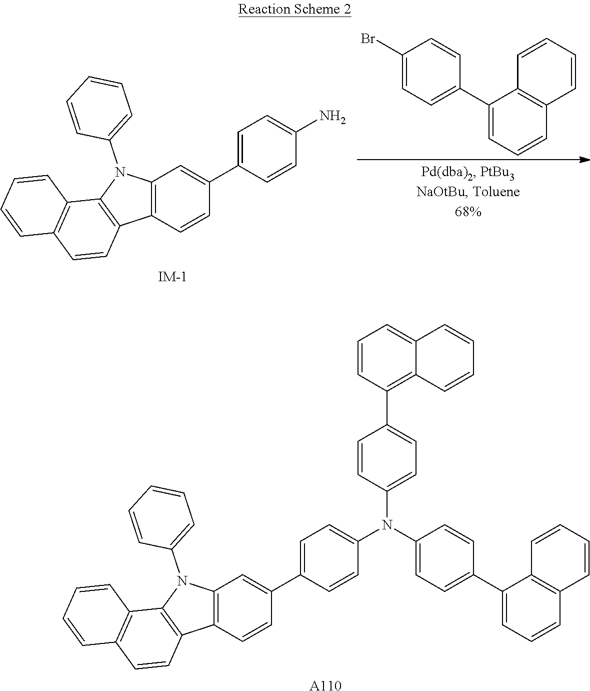

### 1) Synthesis of Compound A110

In an Ar atmosphere, in a 300 mL three-neck flask, Compound IM-1 (10.00 g, 26.0 mmol), Pd(dba)₂ (0.90 g, 0.06 equiv, 1.6 mmol), NaO^{t}Bu (7.50 g, 3.0 equiv, 78.0 mmol), toluene (130 mL), 1-(4-bromophenyl)naphthalene (18.41 g, 2.5 equiv, 65.0 mmol), and P^{t}Bu₃ (1.05 g, 0.2 equiv, 5.2 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound A110 (13.95 g, yield 68%).

By measuring FAB-MS, a mass number of m/z=788 was observed by molecular ion peak, thereby identifying Compound A110.

### (3) Synthesis of Compound A152

Amine Compound A152 according to an example may be synthesized by, for example, the steps shown in Reaction Scheme 3:

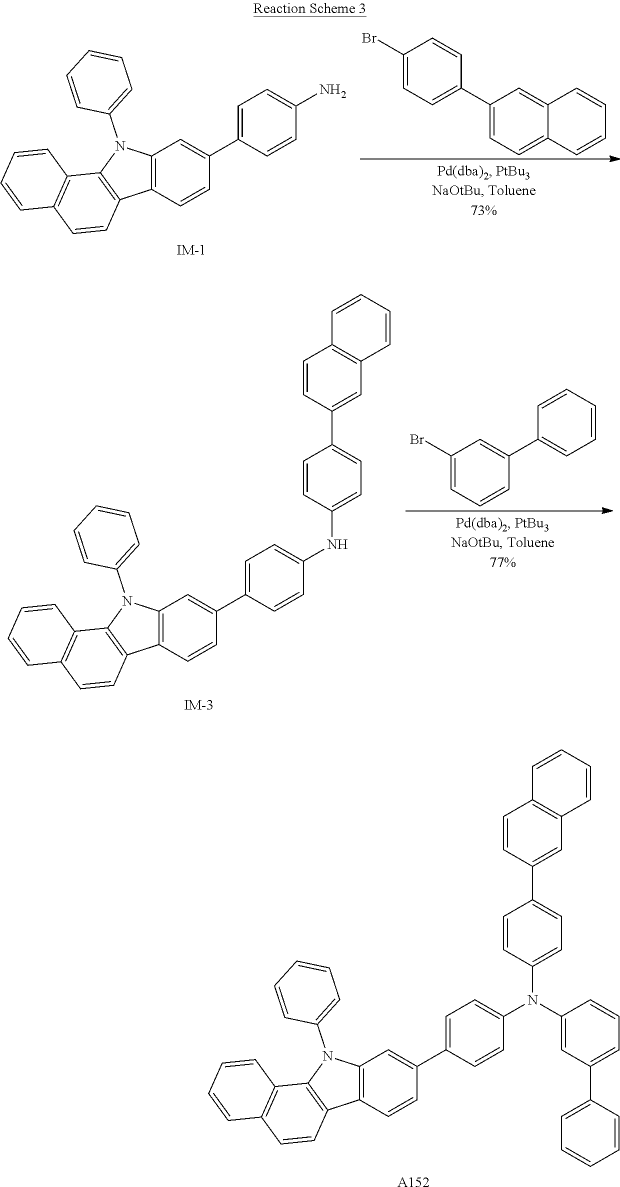

### 1) Synthesis of Compound IM-3

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 2-(4-bromophenyl)naphthalene (12.15 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-3 (16.71 g, yield 73%).

By measuring FAB-MS, a mass number of m/z=586 was observed by molecular ion peak, thereby identifying Compound IM-3.

### 2) Synthesis of Compound A152

In an Ar atmosphere, in a 300 mL three-neck flask, Compound IM-3 (10.00 g, 17.0 mmol), Pd(dba)₂ (0.29 g, 0.03 equiv, 0.5 mmol), NaO^{t}Bu (3.28 g, 2.0 equiv, 34.1 mmol), toluene (85 mL), 3-bromobiphenyl (4.37 g, 1.1 equiv, 18.7 mmol), and Pt^{B}u₃ (0.34 g, 0.1 equiv, 1.7 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound A152 (9.70 g, yield 77%).

By measuring FAB-MS, a mass number of m/z=738 was observed by molecular ion peak, thereby identifying Compound A152.

### (4) Synthesis of Compound B11

Amine Compound B11 according to an example may be synthesized by, for example, the steps shown in Reaction Scheme 4:

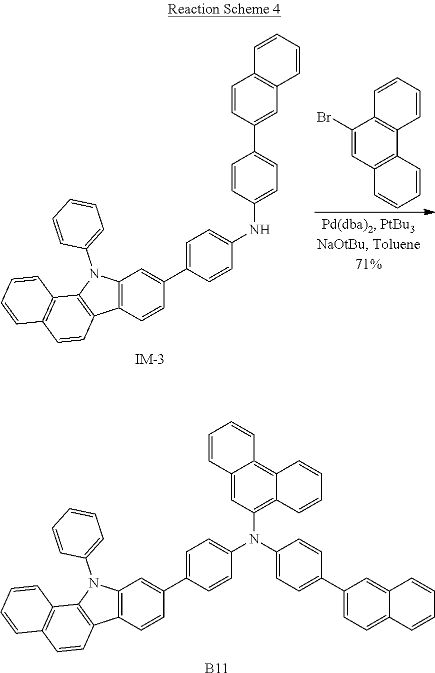

### 1) Synthesis of Compound B11

In an Ar atmosphere, in a 300 mL three-neck flask, Compound IM-3 (10.00 g, 17.0 mmol), Pd(dba)₂ (0.29 g, 0.03 equiv, 0.5 mmol), NaO^{t}Bu (3.28 g, 2.0 equiv, 34.1 mmol), toluene (85 mL), 9-bromophenanthrene (4.82 g, 1.1 equiv, 18.7 mmol), and PtBu₃ (0.34 g, 0.1 equiv, 1.7 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound B11 (9.23 g, yield 71%).

By measuring FAB-MS, a mass number of m/z=762 was observed by molecular ion peak, thereby identifying Compound B11. (5) Synthesis of Compound B130

Amine Compound B130 according to an example may be synthesized by, for example, the steps shown in Reaction Scheme 5:

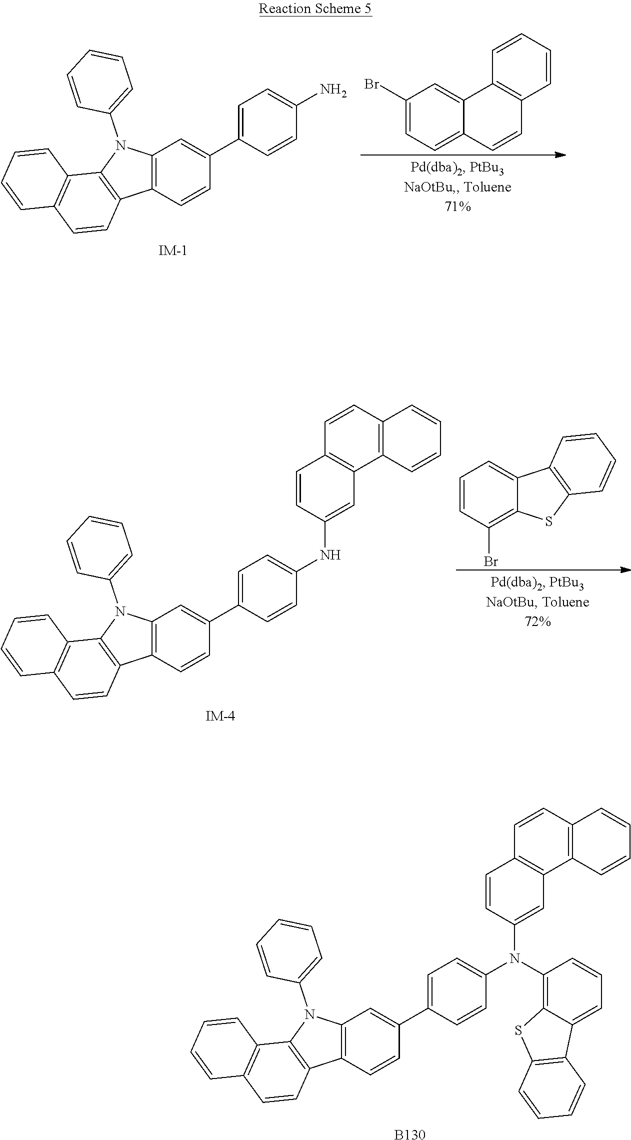

### 1) Synthesis of Compound IM-4

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 3-bromophenanthrene (11.03 g, 1.1 equiv, 42.9 mmol), and P^{t}Bu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-4 (15.53 g, yield 71%).

By measuring FAB-MS, a mass number of m/z=560 was observed by molecular ion peak, thereby identifying Compound IM-4.

### 2) Synthesis of Compound B130

In an Ar atmosphere, in a 300 mL three-neck flask, Compound IM-4 (10.00 g, 17.8 mmol), Pd(dba)₂ (0.31 g, 0.03 equiv, 0.5 mmol), NaO^{t}Bu (3.43 g, 2.0 equiv, 35.7 mmol), toluene (89 mL), 4-bromodibenzothiophene (5.16 g, 1.1 equiv, 19.6 mmol), and PtBu₃ (0.36 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound B130 (9.54 g, yield 72%).

By measuring FAB-MS, a mass number of m/z=742 was observed by molecular ion peak, thereby identifying Compound B130.

### (6) Synthesis of Compound B163

Amine Compound B163 according to an example may be synthesized by, for example, the steps shown in Reaction Scheme 6:

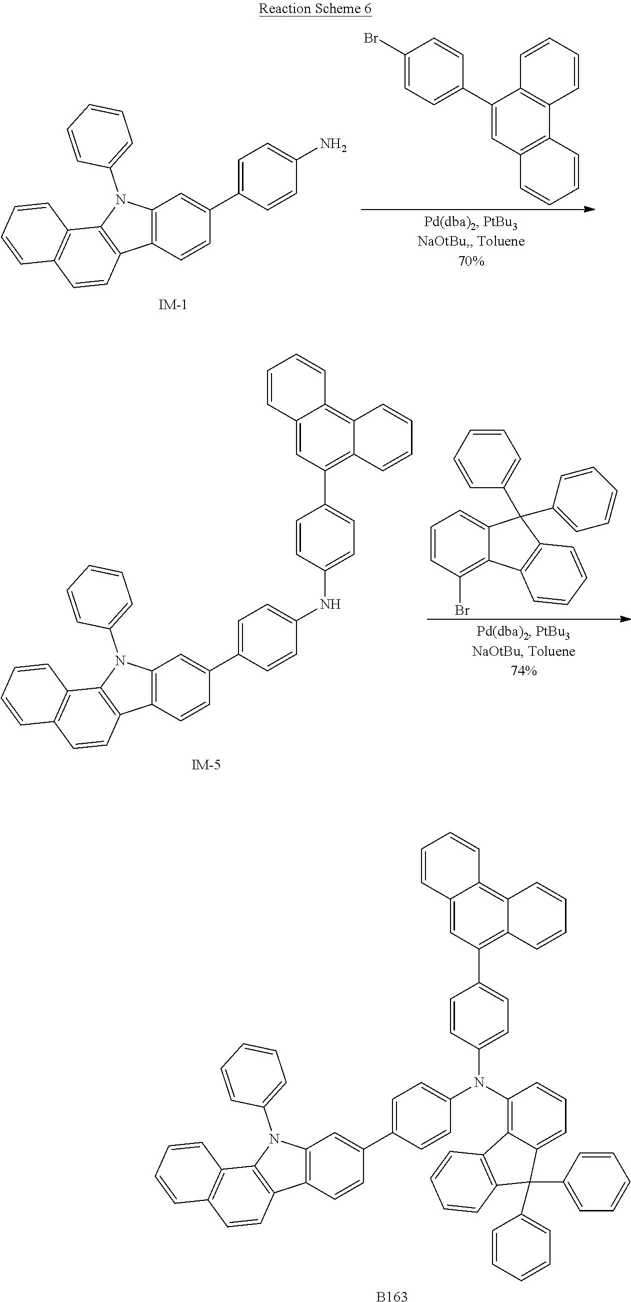

### 1) Synthesis of Compound IM-5

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 9-(4-bromophenyl)phenanthrene (14.30 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-5 (17.39 g, yield 70%).

By measuring FAB-MS, a mass number of m/z=636 was observed by molecular ion peak, thereby identifying Compound IM-5.

### 2) Synthesis of Compound B163

In an Ar atmosphere, in a 300 mL three-neck flask, Compound IM-5 (10.00 g, 15.7 mmol), Pd(dba)₂ (0.27 g, 0.03 equiv, 0.5 mmol), NaO^{t}Bu (3.02 g, 2.0 equiv, 31.4 mmol), toluene (78 mL), 4-bromo-9,9-diphenyl-9H-fluorene (6.86 g, 1.1 equiv, 17.3 mmol), and PtBu₃ (0.32 g, 0.1 equiv, 1.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound B163 (11.08 g, yield 74%).

By measuring FAB-MS, a mass number of m/z=953 was observed by molecular ion peak, thereby identifying Compound B163.

### (7) Synthesis of Compound B198

Amine Compound B198 according to an example may be synthesized by, for example, the steps shown in Reaction Scheme 7:

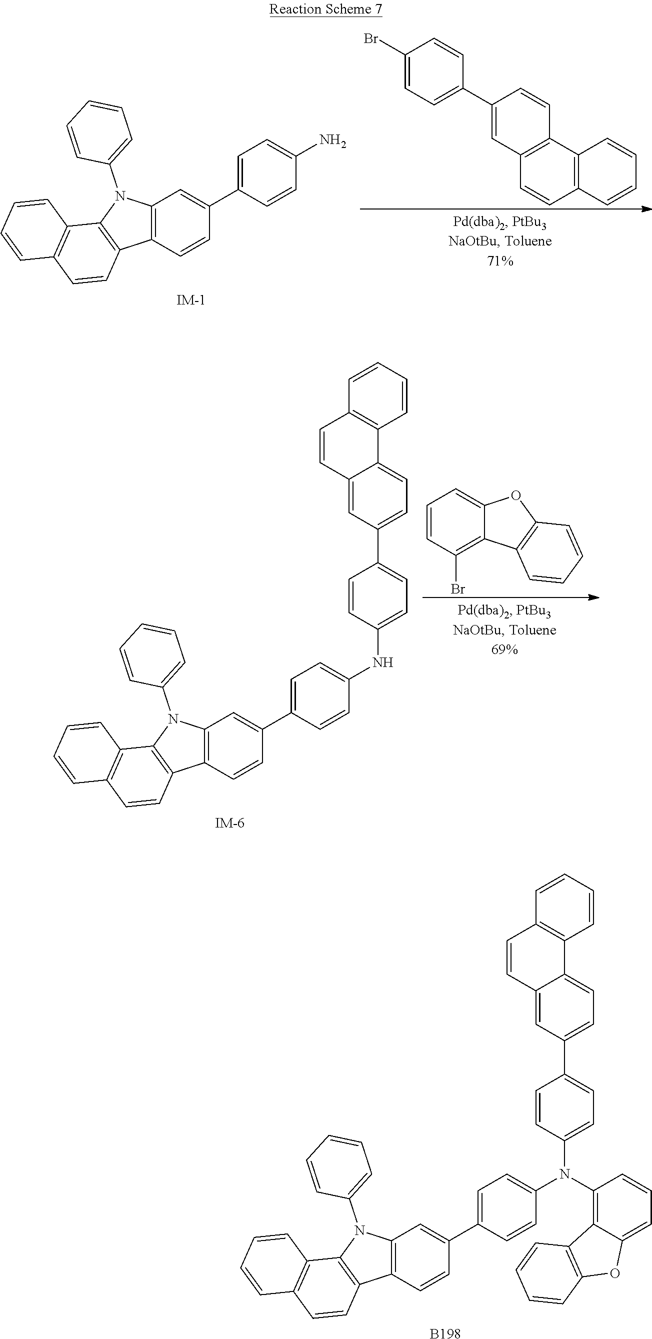

### 1) Synthesis of Compound IM-6

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 2-(4-bromophenyl)phenanthrene (14.30 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-6 (17.64 g, yield 71%).

By measuring FAB-MS, a mass number of m/z=636 was observed by molecular ion peak, thereby identifying Compound IM-6.

### 2) Synthesis of Compound B198

In an Ar atmosphere, in a 300 mL three-neck flask, Compound IM-6 (10.00 g, 15.7 mmol), Pd(dba)₂ (0.27 g, 0.03 equiv, 0.5 mmol), NaO^{t}Bu (3.02 g, 2.0 equiv, 31.4 mmol), toluene (78 mL), 1-bromodibenzofuran (4.27 g, 1.1 equiv, 17.3 mmol), and P^{t}Bu₃ (0.32 g, 0.1 equiv, 1.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound B198 (8.70 g, yield 69%).

By measuring FAB-MS, a mass number of m/z=802 was observed by molecular ion peak, thereby identifying Compound B198.

### (8) Synthesis of Compound C1

Amine Compound C1 according to an example may be synthesized by, for example, the steps shown in Reaction Scheme 8:

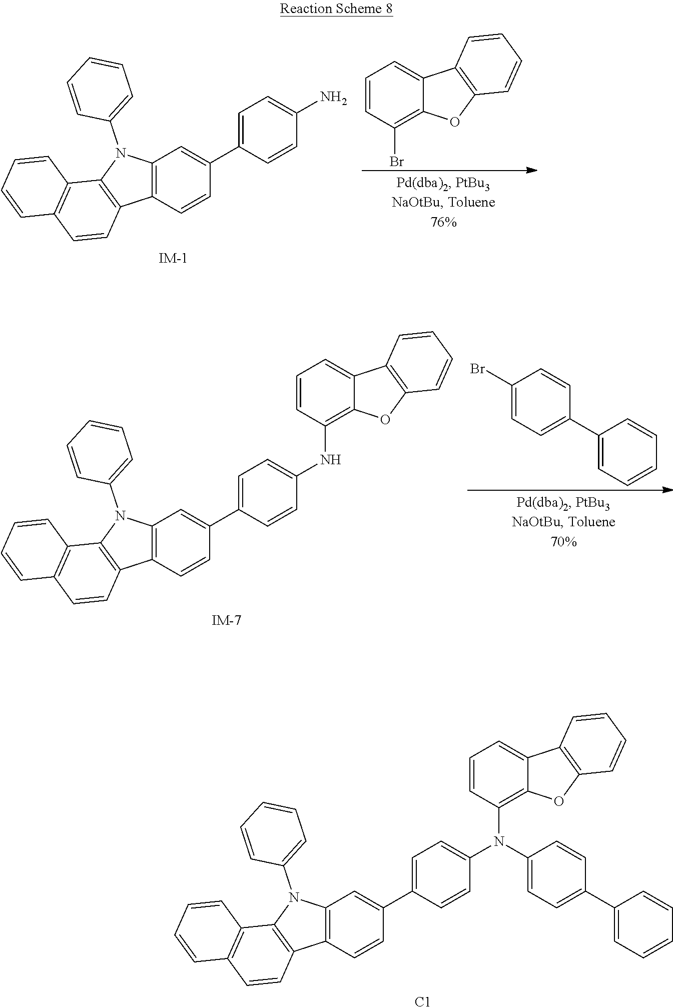

### 1) Synthesis of Compound IM-7

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 4-bromodibenzofuran (10.60 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-7 (16.33 g, yield 76%).

By measuring FAB-MS, a mass number of m/z=550 was observed by molecular ion peak, thereby identifying Compound IM-7.

### 2) Synthesis of Compound Cl

In an Ar atmosphere, in a 300 mL three-neck flask, Compound IM-7 (10.00 g, 18.2 mmol), Pd(dba)₂ (0.31 g, 0.03 equiv, 0.5 mmol), NaO^{t}Bu (3.49 g, 2.0 equiv, 36.3 mmol), toluene (91 mL), 4-bromo-biphenyl (4.66 g, 1.1 equiv, 20.0 mmol), and PtBu₃ (0.37 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound C1 (8.93 g, yield 70%).

By measuring FAB-MS, a mass number of m/z=702 was observed by molecular ion peak, thereby identifying Compound C1.

### (9) Synthesis of Compound C35

Amine Compound C35 according to an example may be synthesized by, for example, the steps shown in Reaction Scheme 9:

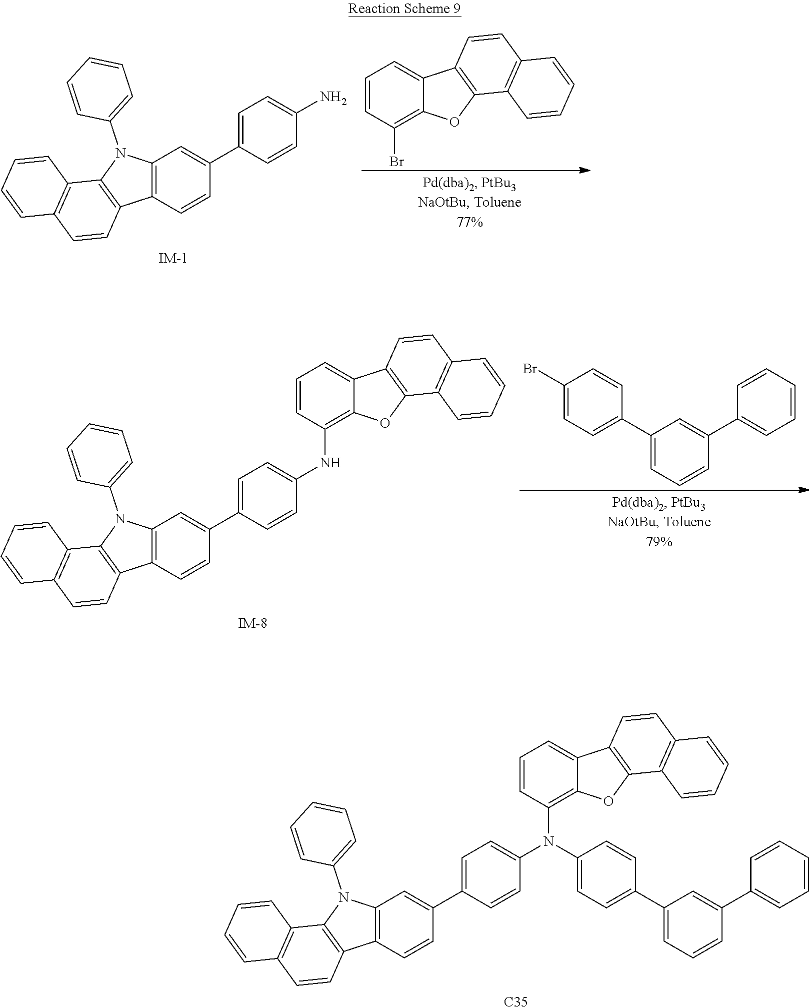

### 1) Synthesis of Compound IM-8

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 10-bromonaphtho[1,2-b]benzofuran (12.75 g, 1.1 equiv, 42.9 mmol), and P^{t}Bu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-8 (18.05 g, yield 77%).

By measuring FAB-MS, a mass number of m/z=600 was observed by molecular ion peak, thereby identifying Compound IM-8.

### 2) Synthesis of Compound C35

In an Ar atmosphere, in a 300 mL three-neck flask, Compound IM-8 (10.00 g, 16.6 mmol), Pd(dba)₂ (0.29 g, 0.03 equiv, 0.5 mmol), NaO^{t}Bu (3.20 g, 2.0 equiv, 33.3 mmol), toluene (83 mL), 4-bromo-1,1′:3′,1″-terphenyl (5.66 g, 1.1 equiv, 18.3 mmol), and P^{t}Bu₃ (0.34 g, 0.1 equiv, 1.7 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound C35 (10.90 g, yield 79%).

By measuring FAB-MS, a mass number of m/z=829 was observed by molecular ion peak, thereby identifying Compound C35.

### (10) Synthesis of Compound C47

Amine Compound C47 according to an example may be synthesized by, for example, the steps shown in Reaction Scheme 10:

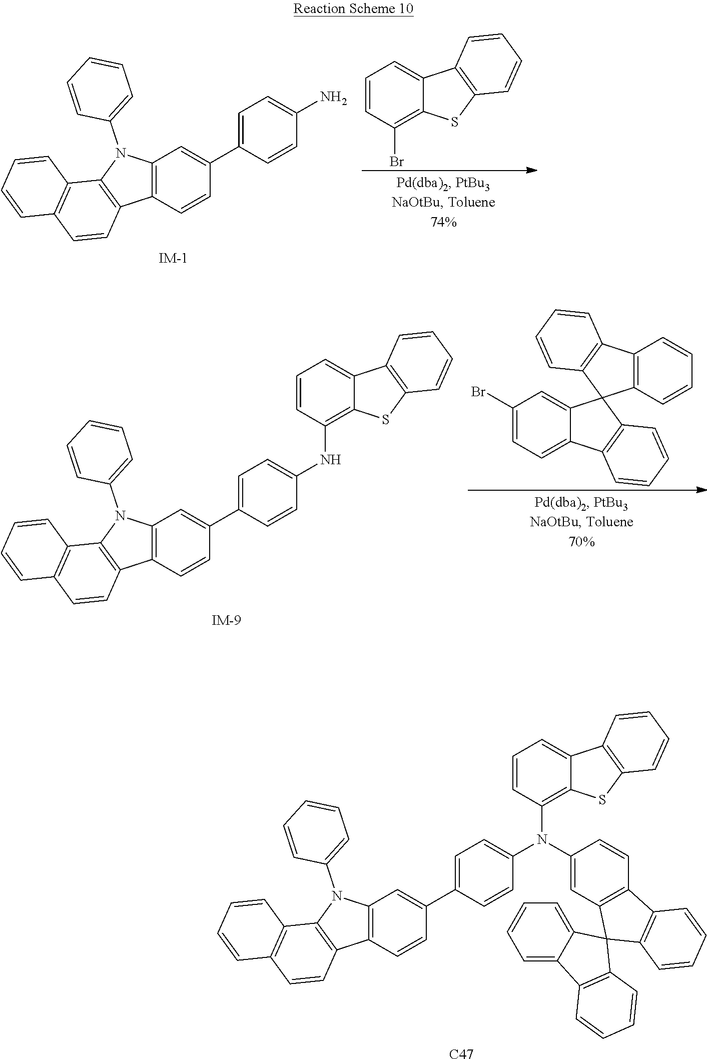

### 1) Synthesis of Compound IM-9

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 4-bromodibenzothiophene (11.29 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-9 (16.36 g, yield 74%).

By measuring FAB-MS, a mass number of m/z=566 was observed by molecular ion peak, thereby identifying Compound IM-9.

### 2) Synthesis of Compound C47

In an Ar atmosphere, in a 300 mL three-neck flask, Compound IM-9 (10.00 g, 17.6 mmol), Pd(dba)₂ (0.30 g, 0.03 equiv, 0.5 mmol), NaO^{t}Bu (3.39 g, 2.0 equiv, 35.3 mmol), toluene (88 mL), 2-bromo-9,9′-spirobi[fluorene] (7.67 g, 1.1 equiv, 19.4 mmol), and P^{t}Bu₃ (0.36 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound C47 (10.88 g, yield 70%).

By measuring FAB-MS, a mass number of m/z=881 was observed by molecular ion peak, thereby identifying Compound C47.

### (11) Synthesis of Compound C90

Amine Compound C90 according to an example may be synthesized by, for example, the steps shown in Reaction Scheme 11:

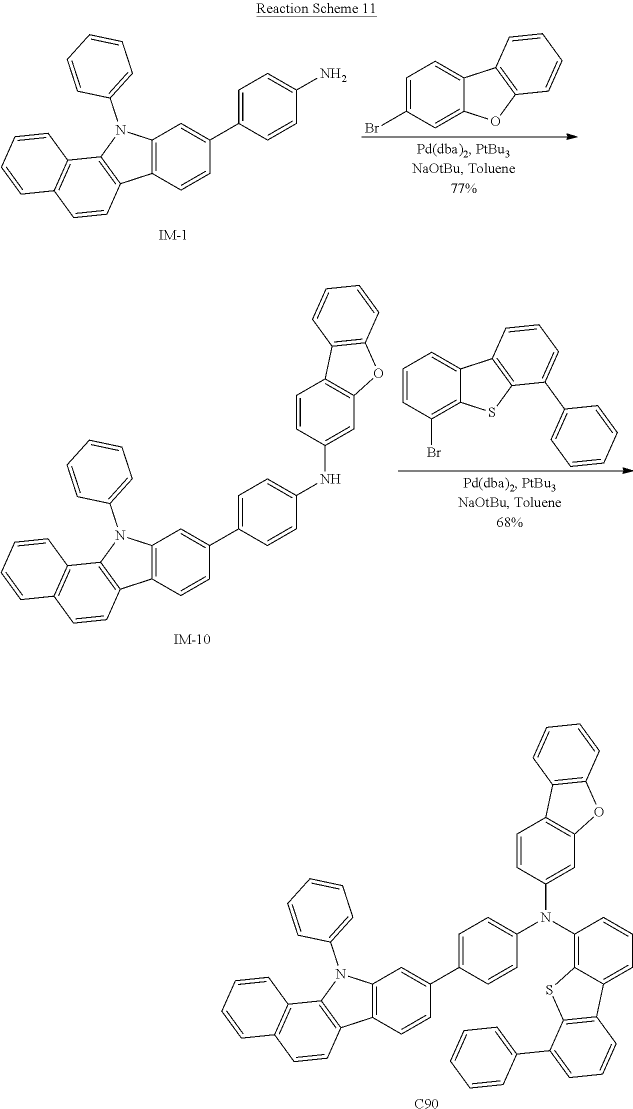

### 1) Synthesis of Compound IM-10

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 3-bromodibenzofuran (10.60 g, 1.1 equiv, 42.9 mmol), and P^{t}Bu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-10 (16.54 g, yield 77%).

By measuring FAB-MS, a mass number of m/z=550 was observed by molecular ion peak, thereby identifying Compound IM-10.

### 2) Synthesis of Compound C90

In an Ar atmosphere, in a 300 mL three-neck flask, Compound IM-10 (10.00 g, 18.2 mmol), Pd(dba)₂ (0.31 g, 0.03 equiv, 0.5 mmol), NaO^{t}Bu (3.49 g, 2.0 equiv, 36.3 mmol), toluene (91 mL), 4-bromo-6-phenyldibenzothiophene (6.78 g, 1.1 equiv, 20.0 mmol), and P^{t}Bu₃ (0.37 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound C90 (9.99 g, yield 68%).

By measuring FAB-MS, a mass number of m/z=809 was observed by molecular ion peak, thereby identifying Compound C90.

### (12) Synthesis of Compound C175

Amine Compound C175 according to an example may be synthesized by, for example, the steps shown in Reaction Scheme 12:

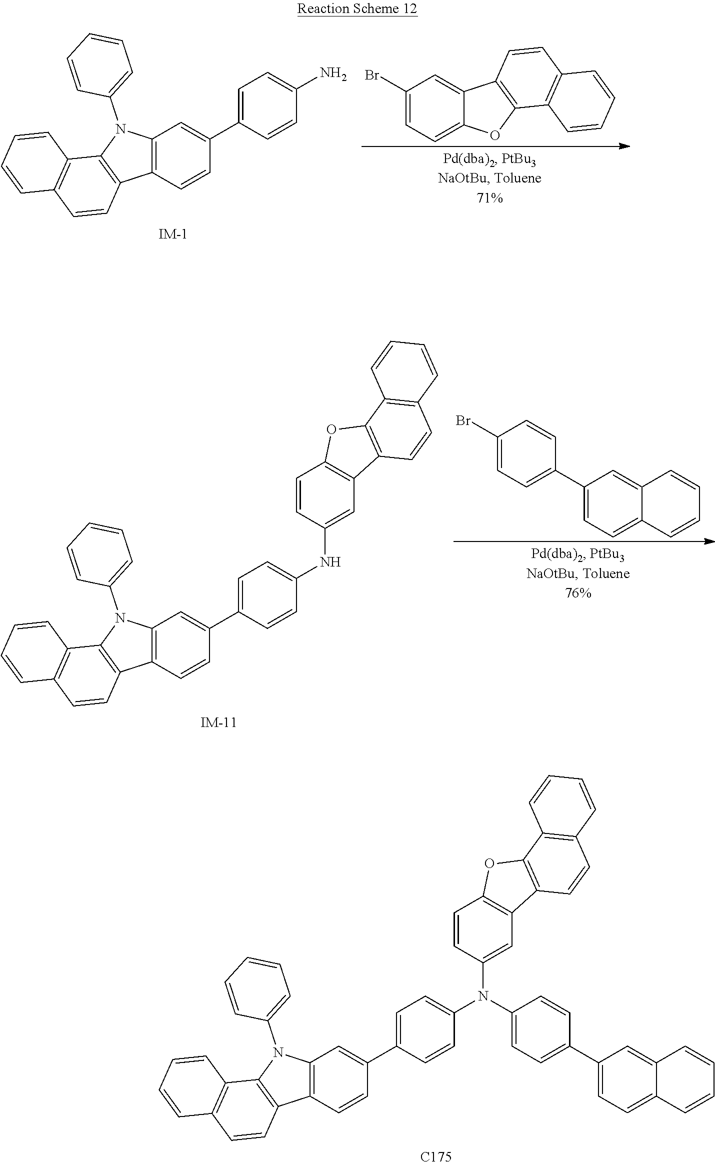

### 1) Synthesis of Compound IM-11

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 8-bromonaphtho[1,2-b]benzofuran (12.75 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-11 (16.64 g, yield 71%).

By measuring FAB-MS, a mass number of m/z=600 was observed by molecular ion peak, thereby identifying Compound IM-11.

### 2) Synthesis of Compound C175

In an Ar atmosphere, in a 300 mL three-neck flask, Compound IM-11 (10.00 g, 16.6 mmol), Pd(dba)₂ (0.29 g, 0.03 equiv, 0.5 mmol), NaO^{t}Bu (3.20 g, 2.0 equiv, 33.3 mmol), toluene (83 mL), 2-(4-bromophenyl)naphthalene (5.16 g, 1.1 equiv, 18.3 mmol), and P^{t}Bu₃ (0.34 g, 0.1 equiv, 1.7 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound C175 (10.16 g, yield 76%).

By measuring FAB-MS, a mass number of m/z=802 was observed by molecular ion peak, thereby identifying Compound C175.

### (13) Synthesis of Compound C214

Amine Compound C214 according to an example may be synthesized by, for example, the steps shown in Reaction Scheme 13:

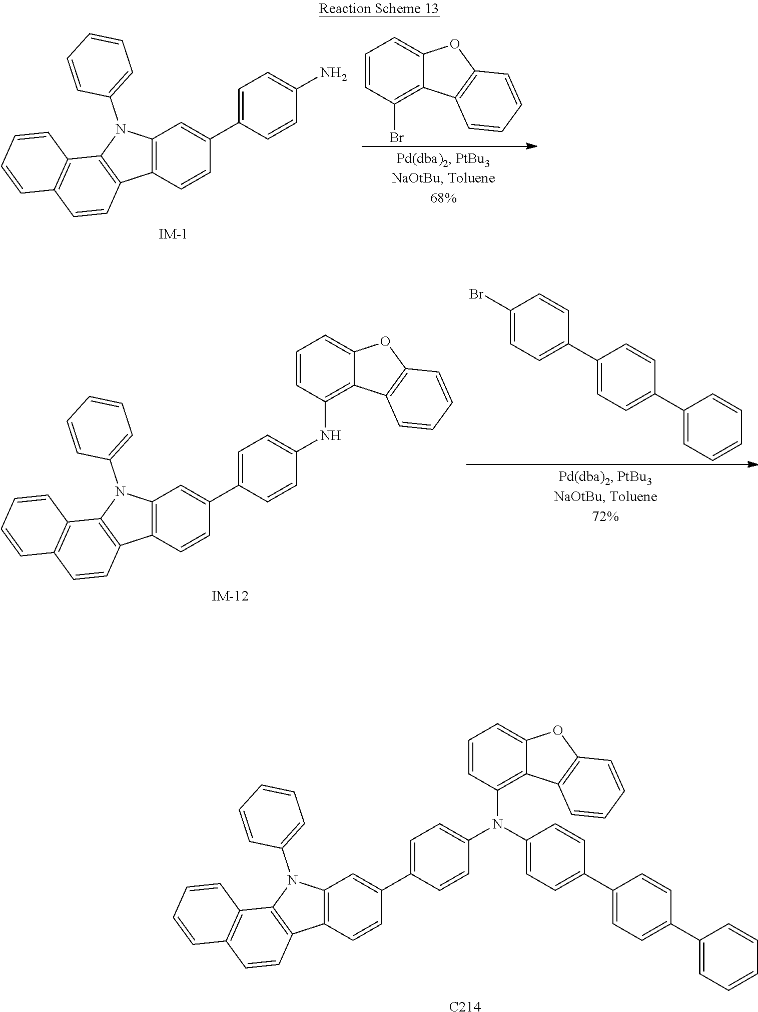

### 1) Synthesis of Compound IM-12

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 1-bromodibenzofuran (10.60 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-12 (14.61 g, yield 68%).

By measuring FAB-MS, a mass number of m/z=550 was observed by molecular ion peak, thereby identifying Compound IM-12.

### 2) Synthesis of Compound C214

In an Ar atmosphere, in a 300 mL three-neck flask, Compound IM-12 (10.00 g, 18.2 mmol), Pd(dba)₂ (0.31 g, 0.03 equiv, 0.5 mmol), NaO^{t}Bu (3.49 g, 2.0 equiv, 36.3 mmol), toluene (91 mL), 4-bromo-1,1′:4′,1″-terphenyl (6.18 g, 1.1 equiv, 20.0 mmol), and PtBu₃ (0.37 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound C214 (10.18 g, yield 72%).

By measuring FAB-MS, a mass number of m/z=809 was observed by molecular ion peak, thereby identifying Compound C214.

### (14) Synthesis of Compound C293

Amine Compound C293 according to an example may be synthesized by, for example, the steps shown in Reaction Scheme 14:

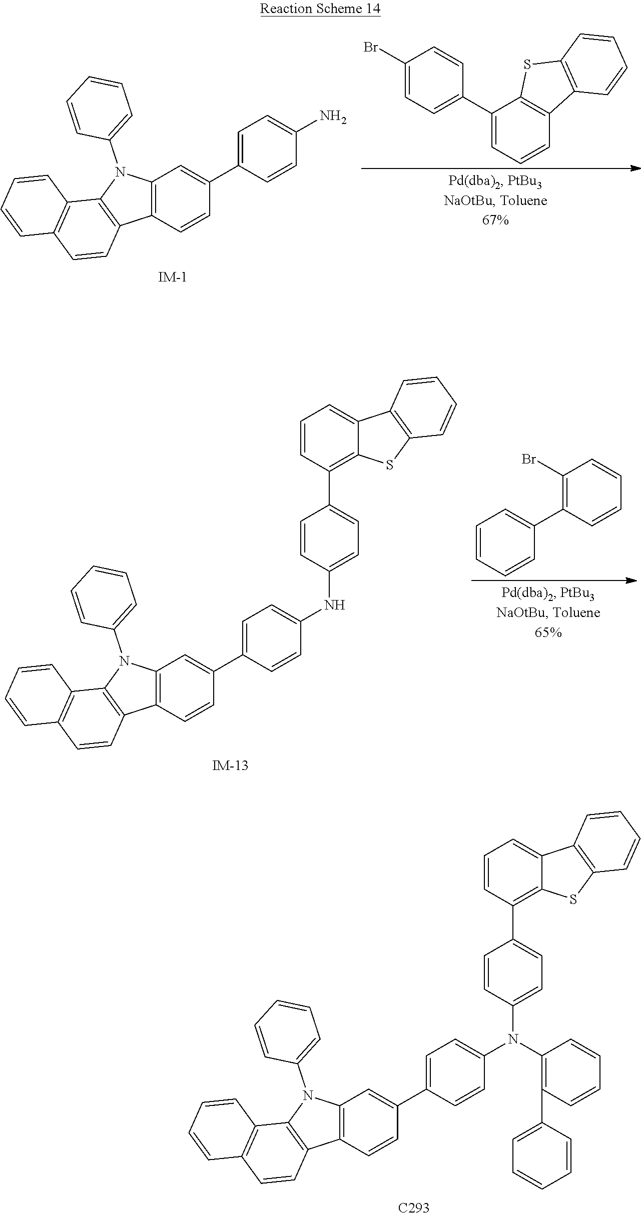

### 1) Synthesis of Compound IM-13

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL),4-(4-bromophenyl)dibenzothiophene (14.56 g, 1.1 equiv, 42.9 mmol), and P^{t}Bu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-13 (16.80 g, yield 67%).

By measuring FAB-MS, a mass number of m/z=642 was observed by molecular ion peak, thereby identifying Compound IM-13.

### 2) Synthesis of Compound C293

In an Ar atmosphere, in a 300 mL three-neck flask, Compound IM-13 (10.00 g, 15.6 mmol), Pd(dba)₂ (0.27 g, 0.03 equiv, 0.5 mmol), NaO^{t}Bu (2.99 g, 2.0 equiv, 31.1 mmol), toluene (78 mL), 2-bromobiphenyl (3.99 g, 1.1 equiv, 17.1 mmol), and PtBu₃ (0.31 g, 0.1 equiv, 1.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound C293 (8.04 g, yield 65%).

By measuring FAB-MS, a mass number of m/z=795 was observed by molecular ion peak, thereby identifying Compound C293.

### (15) Synthesis of Compound C328

Amine Compound C328 according to an example may be synthesized by, for example, the steps shown in Reaction Scheme 15:

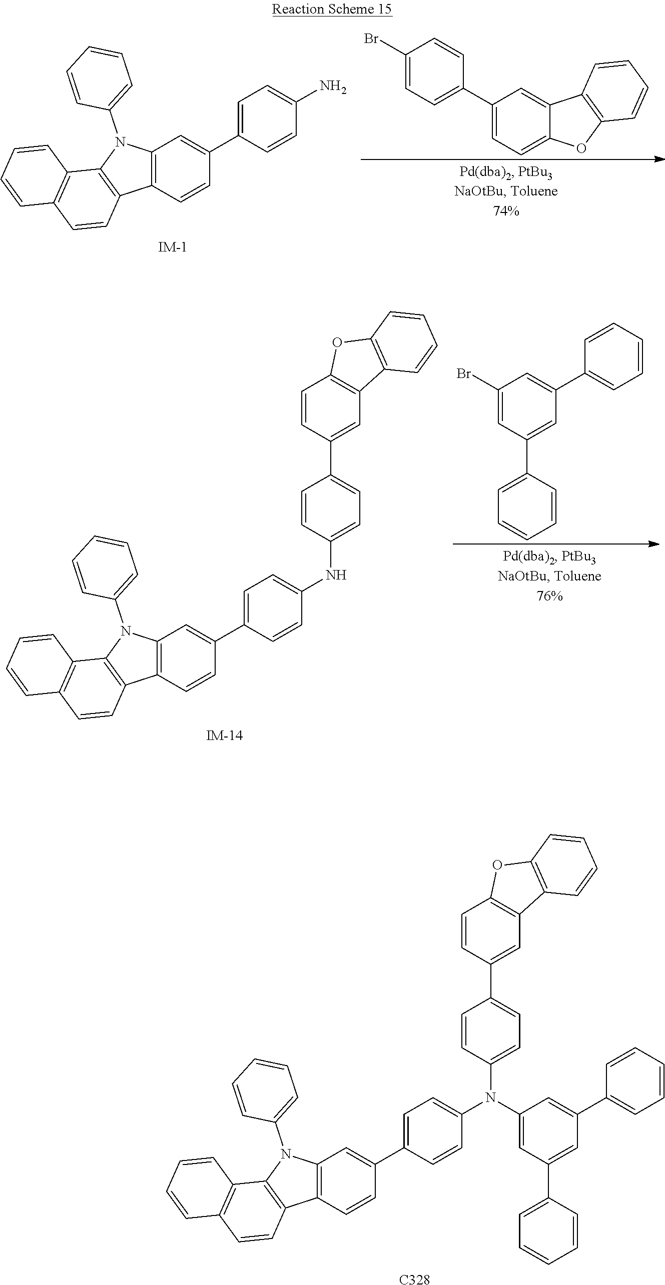

### 1) Synthesis of Compound IM-14

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 2-(4-bromophenyl)dibenzofuran (13.87 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-14 (18.09 g, yield 74%).

By measuring FAB-MS, a mass number of m/z=626 was observed by molecular ion peak, thereby identifying Compound IM-14.

### 2) Synthesis of Compound C328

In an Ar atmosphere, in a 300 mL three-neck flask, Compound IM-14 (10.00 g, 16.0 mmol), Pd(dba)₂ (0.28 g, 0.03 equiv, 0.5 mmol), NaO^{t}Bu (3.07 g, 2.0 equiv, 31.9 mmol), toluene (80 mL), 5′-bromo-1,1′:3′,1″-terphenyl (5.43 g, 1.1 equiv, 17.6 mmol), and P^{t}Bu₃ (0.32 g, 0.1 equiv, 1.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound C328 (10.37 g, yield 76%).

By measuring FAB-MS, a mass number of m/z=855 was observed by molecular ion peak, thereby identifying Compound C328.

### (16) Synthesis of Compound D16

Amine Compound D16 according to an example may be synthesized by, for example, the steps shown in Reaction Scheme 16:

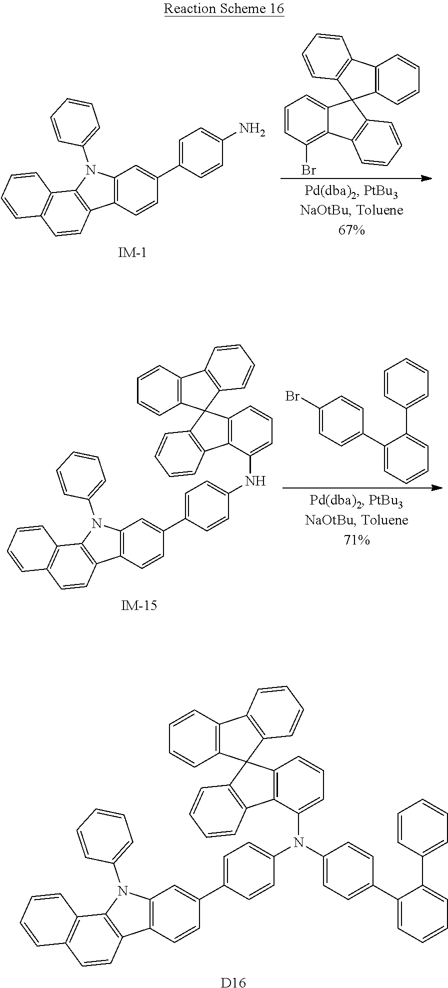

### 1) Synthesis of Compound IM-15

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 4-bromo-9,9′-spirobi[fluorene] (16.96 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-15 (18.27 g, yield 67%).

By measuring FAB-MS, a mass number of m/z=698 was observed by molecular ion peak, thereby identifying Compound IM-15.

### 2) Synthesis of Compound D16

In an Ar atmosphere, in a 300 mL three-neck flask, Compound IM-15 (10.00 g, 14.3 mmol), Pd(dba)₂ (0.25 g, 0.03 equiv, 0.4 mmol), NaO^{t}Bu (2.75 g, 2.0 equiv, 28.6 mmol), toluene (72 mL), 4-bromo-1,1′:2′,1″-terphenyl (4.87 g, 1.1 equiv, 15.7 mmol), and PtBu₃ (0.29 g, 0.1 equiv, 1.4 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound D16 (9.42 g, yield 71%).

By measuring FAB-MS, a mass number of m/z=927 was observed by molecular ion peak, thereby identifying Compound D16.

### (17) Synthesis of Compound D60

Amine Compound D60 according to an example may be synthesized by, for example, the steps shown in Reaction Scheme 17:

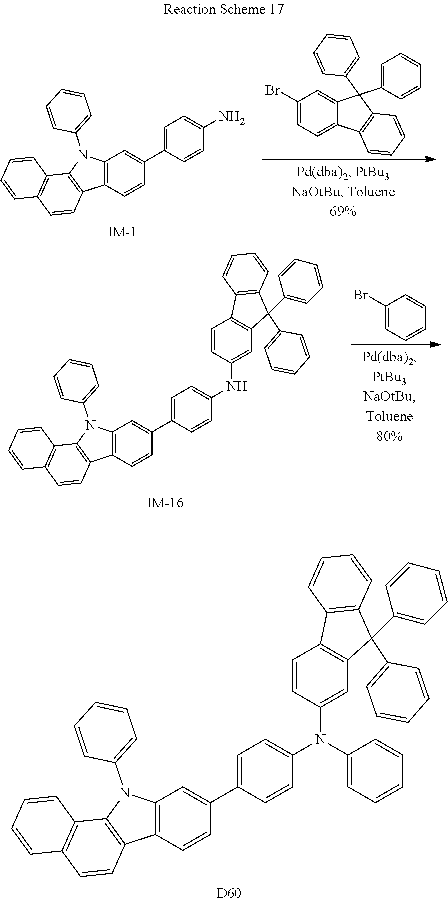

### 1) Synthesis of Compound IM-16

In an Ar atmosphere, in a 500 mL three-neck flask, Compound IM-1 (15.00 g, 39.0 mmol), Pd(dba)₂ (0.67 g, 0.03 equiv, 1.2 mmol), NaO^{t}Bu (3.75 g, 1.0 equiv, 39.0 mmol), toluene (195 mL), 2-bromo-9,9-diphenyl-9H-fluorene (17.05 g, 1.1 equiv, 42.9 mmol), and PtBu₃ (0.79 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Compound IM-16 (18.87 g, yield 69%).

By measuring FAB-MS, a mass number of m/z=700 was observed by molecular ion peak, thereby identifying Compound IM-16.

### 2) Synthesis of Compound D60

In an Ar atmosphere, in a 300 mL three-neck flask, Compound IM-16 (10.00 g, 14.3 mmol), Pd(dba)₂ (0.25 g, 0.03 equiv, 0.4 mmol), NaO^{t}Bu (2.75 g, 2.0 equiv, 28.6 mmol), toluene (72 mL), bromobenzene (2.46 g, 1.1 equiv, 15.7 mmol), and PtBu₃ (0.29 g, 0.1 equiv, 1.4 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound D60 (8.87 g, yield 80%).

By measuring FAB-MS, a mass number of m/z=776 was observed by molecular ion peak, thereby identifying Compound D60.

### 2. Manufacture and Evaluation of Light Emitting Element

Evaluation of the light emitting elements including compounds of Examples and Comparative Examples in a hole transport layer was performed as follows. The method for manufacturing the light emitting element for the evaluation of the element is described below.

### (1) Manufacture of Light Emitting Element 1

A 1,500 A-thick ITO was patterned on a glass substrate, and then the glass substrate was washed with ultrapure water and treated with UV and ozone for about 10 minutes to form a first electrode. Thereafter, 2-TNATA was deposited to form a 600 Å-thick hole injection layer. Then, Example Compound or Comparative Example Compound was deposited to form a 300 Å-thick hole transport layer.

Thereafter, TBP was doped to ADN by 3% to form a 250 Å-thick emission layer. Then, Alq3 was deposited to form a 250 Å-thick electron transport layer, and LiF was deposited to form a 10 Å-thick electron injection layer.

Then, aluminum (Al) was provided to form a 1,000 Å-thick second electrode.

In the Examples, the hole injection layer, the hole transport layer, the emission layer, the electron transport layer, the electron injection layer, and the second electrode were formed by using a vacuum deposition apparatus.

### (2) Manufacture of Light Emitting Element 2

A 1,500 Å-thick ITO was patterned on a glass substrate, and then the glass substrate was washed with ultrapure water and treated with UV and ozone for about 10 minutes to form a first electrode. Thereafter, 2-TNATA was deposited to form a 600 Å-thick hole injection layer. Then, H-1-1 was deposited to form a 200 Å-thick hole transport layer, and then Example Compound or Comparative Example Compound was deposited to form 100 Å-thick electron blocking layer.

Thereafter, TBP was doped to ADN by 3% to form a 250 Å-thick emission layer. Then, Alq3 was deposited to form a 250 Å-thick electron transport layer, and LiF was deposited to form a 10 Å-thick electron injection layer.

Then, aluminum (Al) was provided to form a 1,000 Å-thick second electrode.

In the Examples, the hole injection layer, the hole transport layer, the electron blocking layer, the emission layer, the electron transport layer, the electron injection layer, and the second electrode were formed by using a vacuum deposition apparatus.

Meanwhile, for the molecular weight of Example Compound, FAB-MS was measured by using JMS-700V manufactured by JEOL, Ltd. In addition, for the NMR of Example Compound, 1H-NMR was measured by using AVAVCE300M manufactured by Bruker Biospin K.K. In the following evaluation of the light emitting elements, current densities, voltages and luminous efficiencies of the elements were measured in a dark room by using 2400 Series Source Meter manufactured by Keithley Instruments, Inc., CS-200, Color and Luminance Meter manufactured by Konica Minolta, Inc., PC Program LaVIEW 8.2 for the measurement manufactured by Japan National Instrument, Inc.

Example Compounds and Comparative Example Compounds used to manufacture light emitting element 1 and light emitting element 2 are as follows:

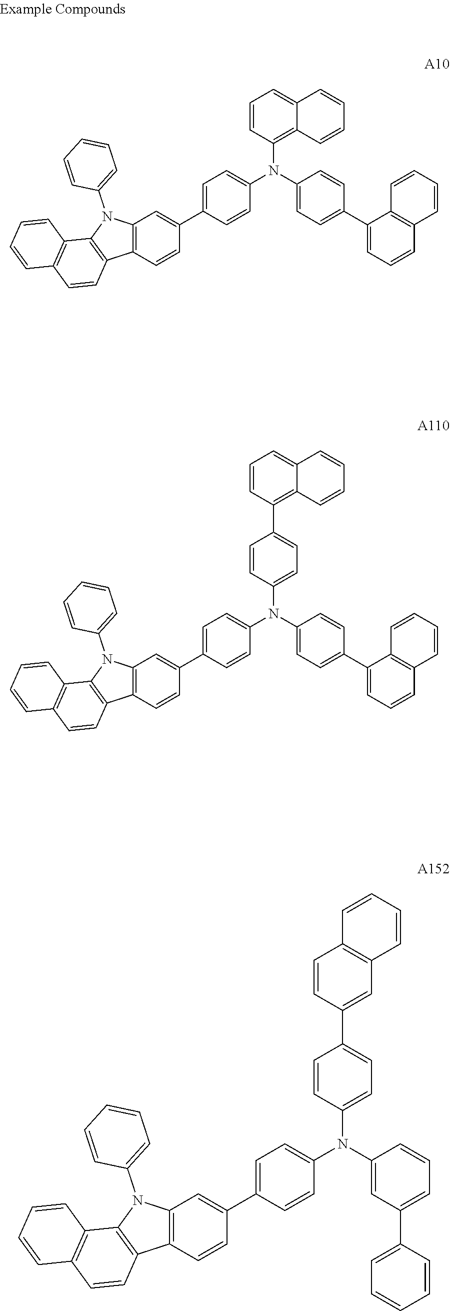 [SMILES: c1ccc(-n2c3cc(-c4ccc(N(c5ccc(-c6cccc7ccccc67)cc5)c5cccc6ccccc56)cc4)ccc3c3ccc4ccccc4c32)cc1 ; c1ccc(-n2c3cc(-c4ccc(N(c5ccc(-c6cccc7ccccc67)cc5)c5ccc(-c6cccc7ccccc67)cc5)cc4)ccc3c3ccc4ccccc4c32)cc1 ; c1ccc(-c2cccc(N(c3ccc(-c4ccc5ccccc5c4)cc3)c3ccc(-c4ccc5c6ccc7ccccc7c6n(-c6ccccc6)c5c4)cc3)c2)cc1]

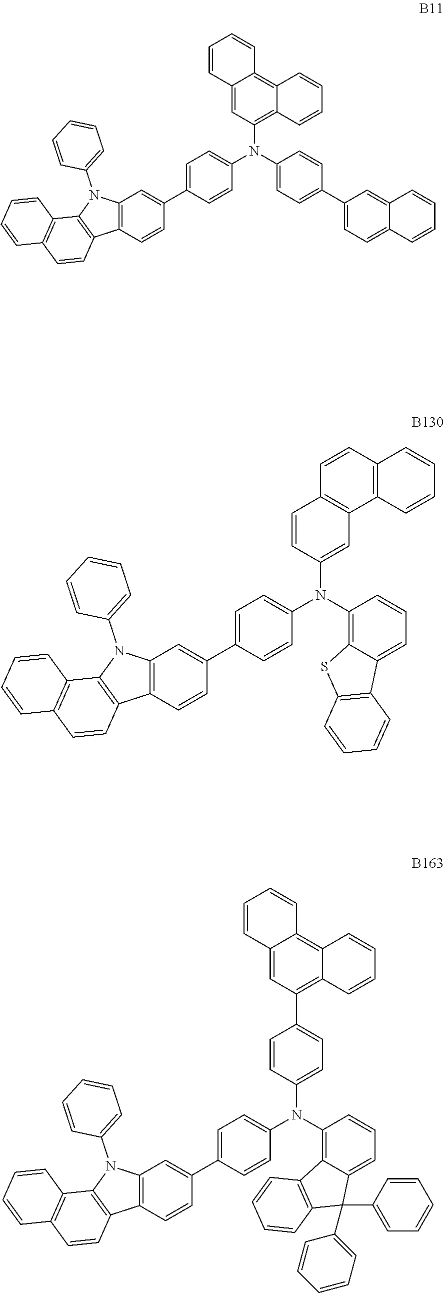 [SMILES: c1ccc(-n2c3cc(-c4ccc(N(c5ccc(-c6ccc7ccccc7c6)cc5)c5cc6ccccc6c6ccccc56)cc4)ccc3c3ccc4ccccc4c32)cc1 ; c1ccc(-n2c3cc(-c4ccc(N(c5ccc6ccc7ccccc7c6c5)c5cccc6c5sc5ccccc56)cc4)ccc3c3ccc4ccccc4c32)cc1 ; c1ccc(-n2c3cc(-c4ccc(N(c5ccc(-c6cc7ccccc7c7ccccc67)cc5)c5cccc6c5-c5ccccc5C6(c5ccccc5)c5ccccc5)cc4)ccc3c3ccc4ccccc4c32)cc1]

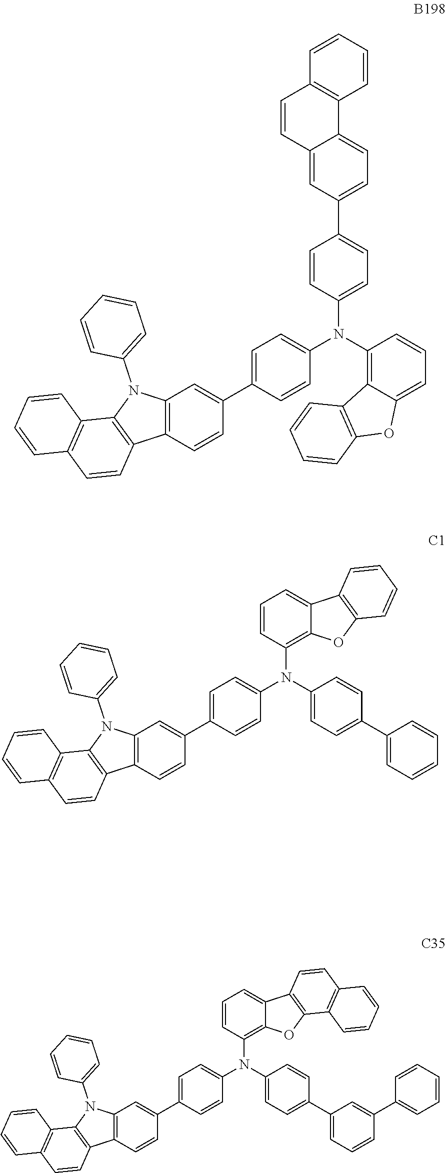 [SMILES: c1ccc(-n2c3cc(-c4ccc(N(c5ccc(-c6ccc7c(ccc8ccccc87)c6)cc5)c5cccc6oc7ccccc7c56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3cccc4c3oc3ccccc34)cc2)cc1 ; c1ccc(-c2cccc(-c3ccc(N(c4ccc(-c5ccc6c7ccc8ccccc8c7n(-c7ccccc7)c6c5)cc4)c4cccc5c4oc4c6ccccc6ccc54)cc3)c2)cc1]

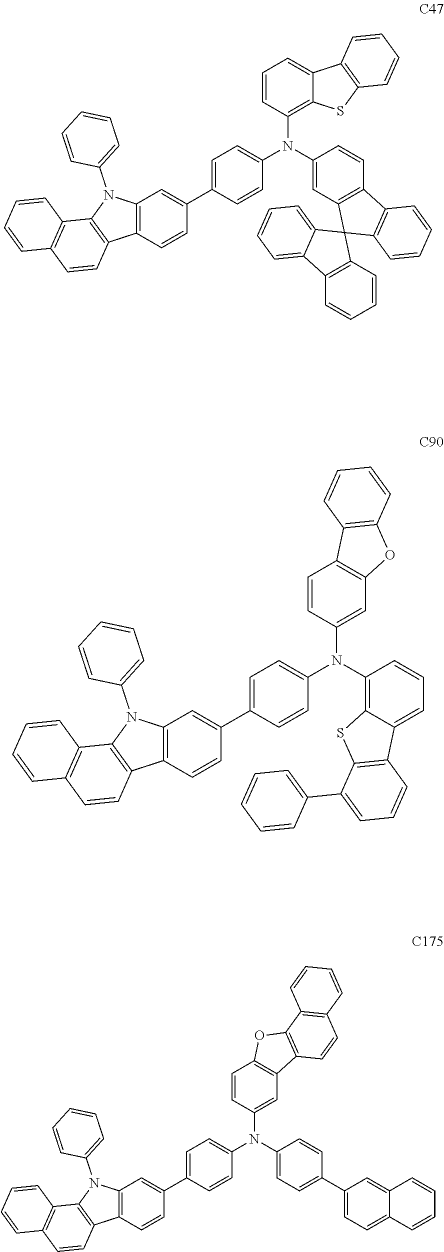 [SMILES: c1ccc(-n2c3cc(-c4ccc(N(c5ccc6c(c5)C5(c7ccccc7-c7ccccc75)c5ccccc5-6)c5cccc6c5sc5ccccc56)cc4)ccc3c3ccc4ccccc4c32)cc1 ; c1ccc(-c2cccc3c2sc2c(N(c4ccc(-c5ccc6c7ccc8ccccc8c7n(-c7ccccc7)c6c5)cc4)c4ccc5c(c4)oc4ccccc45)cccc23)cc1 ; c1ccc(-n2c3cc(-c4ccc(N(c5ccc(-c6ccc7ccccc7c6)cc5)c5ccc6oc7c8ccccc8ccc7c6c5)cc4)ccc3c3ccc4ccccc4c32)cc1]

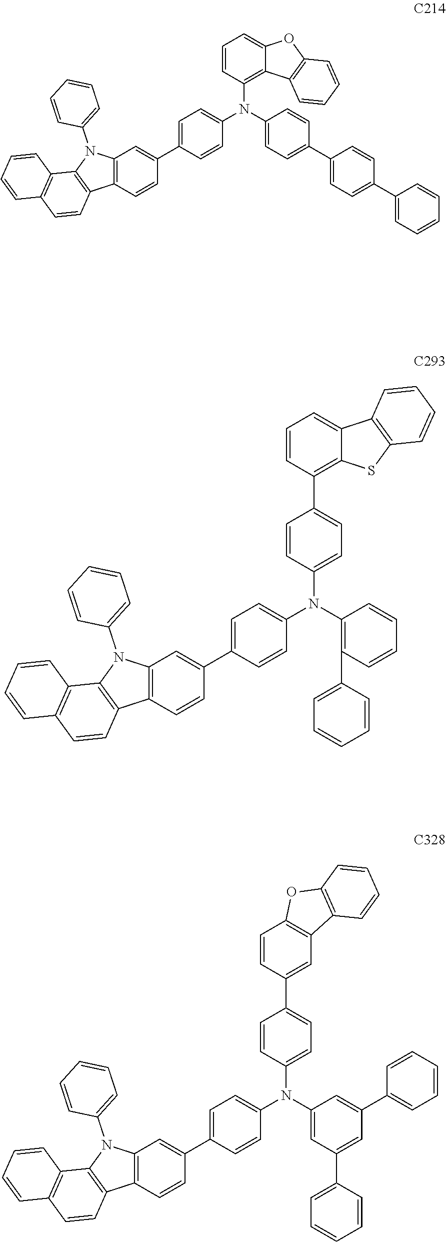 [SMILES: c1ccc(-c2ccc(-c3ccc(N(c4ccc(-c5ccc6c7ccc8ccccc8c7n(-c7ccccc7)c6c5)cc4)c4cccc5oc6ccccc6c45)cc3)cc2)cc1 ; c1ccc(-c2ccccc2N(c2ccc(-c3ccc4c5ccc6ccccc6c5n(-c5ccccc5)c4c3)cc2)c2ccc(-c3cccc4c3sc3ccccc34)cc2)cc1 ; c1ccc(-c2cc(-c3ccccc3)cc(N(c3ccc(-c4ccc5oc6ccccc6c5c4)cc3)c3ccc(-c4ccc5c6ccc7ccccc7c6n(-c6ccccc6)c5c4)cc3)c2)cc1]

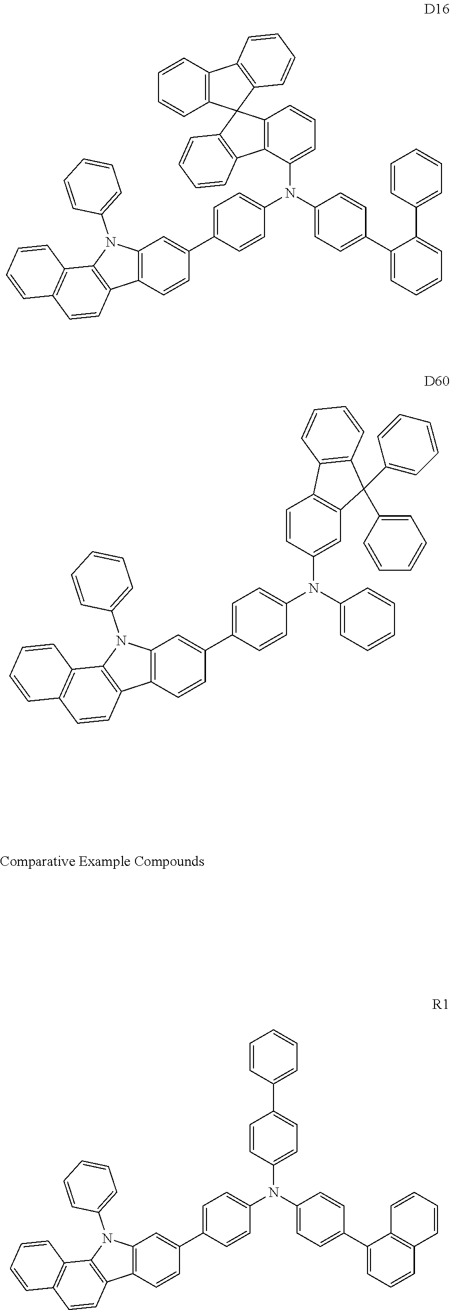 [SMILES: c1ccc(-c2ccccc2-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1 ; c1ccc(N(c2ccc(-c3ccc4c5ccc6ccccc6c5n(-c5ccccc5)c4c3)cc2)c2ccc3c(c2)C(c2ccccc2)(c2ccccc2)c2ccccc2-3)cc1 ; c1ccc(-c2ccc(N(c3ccc(-c4ccc5c6ccc7ccccc7c6n(-c6ccccc6)c5c4)cc3)c3ccc(-c4cccc5ccccc45)cc3)cc2)cc1]

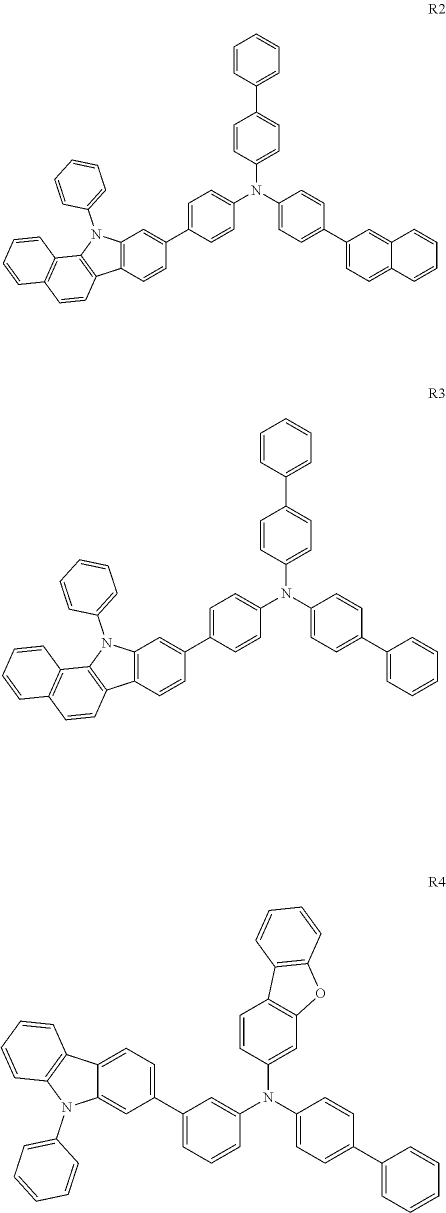 [SMILES: c1ccc(-c2ccc(N(c3ccc(-c4ccc5ccccc5c4)cc3)c3ccc(-c4ccc5c6ccc7ccccc7c6n(-c6ccccc6)c5c4)cc3)cc2)cc1 ; c1ccc(-c2ccc(N(c3ccc(-c4ccccc4)cc3)c3ccc(-c4ccc5c6ccc7ccccc7c6n(-c6ccccc6)c5c4)cc3)cc2)cc1 ; c1ccc(-c2ccc(N(c3cccc(-c4ccc5c6ccccc6n(-c6ccccc6)c5c4)c3)c3ccc4c(c3)oc3ccccc34)cc2)cc1]

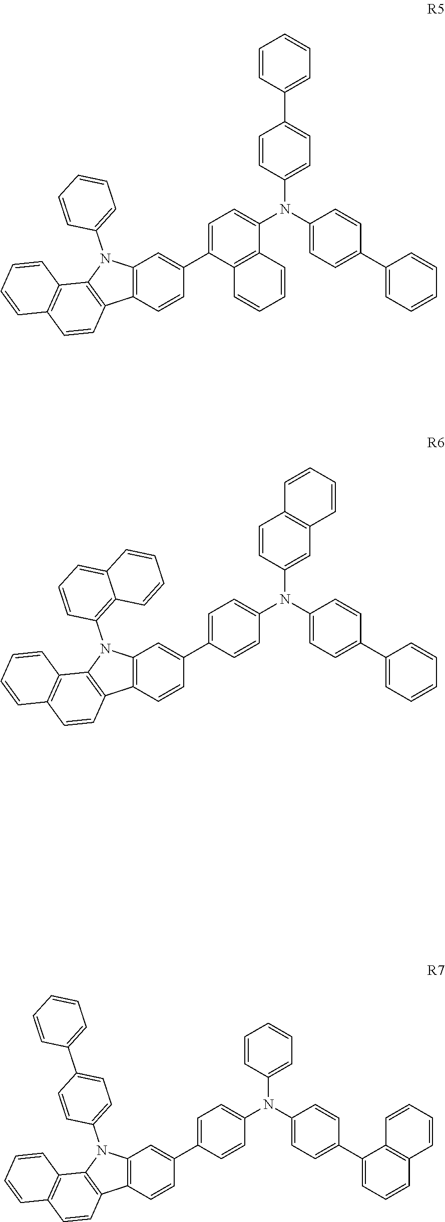 [SMILES: c1ccc(-c2ccc(N(c3ccc(-c4ccccc4)cc3)c3ccc(-c4ccc5c6ccc7ccccc7c6n(-c6ccccc6)c5c4)c4ccccc34)cc2)cc1 ; c1ccc(-c2ccc(N(c3ccc(-c4ccc5c6ccc7ccccc7c6n(-c6cccc7ccccc67)c5c4)cc3)c3ccc4ccccc4c3)cc2)cc1 ; c1ccc(-c2ccc(-n3c4cc(-c5ccc(N(c6ccccc6)c6ccc(-c7cccc8ccccc78)cc6)cc5)ccc4c4ccc5ccccc5c43)cc2)cc1]

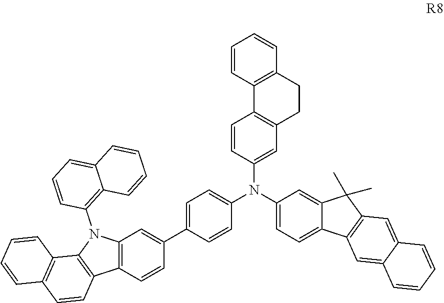 [SMILES: CC1(C)c2cc(N(c3ccc(-c4ccc5c6ccc7ccccc7c6n(-c6cccc7ccccc67)c5c4)cc3)c3ccc4c(c3)CCc3ccccc3-4)ccc2-c2cc3ccccc3cc21]

In some embodiments, compounds of each functional layer used to manufacture light emitting elements 1 and 2 are as follows:

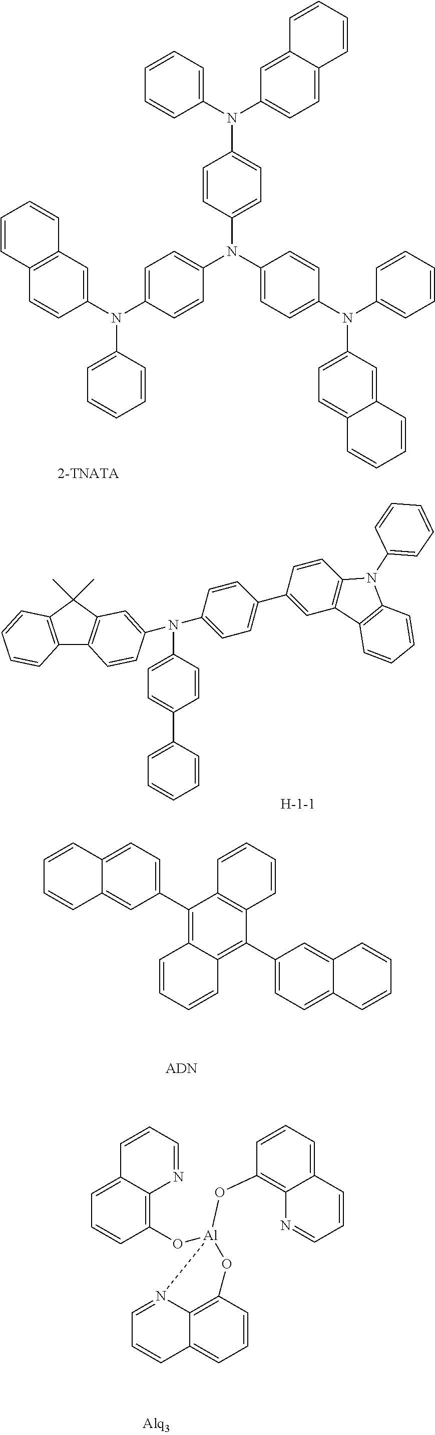

### (3) Evaluation of Light Emitting Element 1 and Light Emitting Element 2

### 1) Evaluation of Light Emitting Element 1

The evaluation results of light emitting element 1 with respect to Examples 1-1 to 1-17 and Comparative Examples 1-1 to 1-8 are listed in Table 1, and the evaluation results of light emitting element 2 with respect to Examples 2-1 to 2-17 and Comparative Examples 2-1 to 2-8 are listed in Table 2. The maximum luminous efficiencies and half service lives of light emitting element 1 and light emitting element 2 are listed in comparison in each of Tables 1 and 2. In the evaluation results of the characteristics for Examples and Comparative Examples listed in Tables 1 and 2, the luminous efficiency shows the efficiency value at a current density of 10 mA/cm².

The element service life is a relative value showing a time when the brightness value is 50% of an initial brightness during the continuous operation of the element at 1,000 cd/m² compared with Comparative Examples 1-3 and 2-3.

The luminous efficiencies and element service lives in Tables 1 and 2 below represent compared values when it is assumed that each of the luminous efficiency and service life of Comparative Examples 1-3 and 2-1, respectively, is 100%.

TABLE 1

| Examples of | | Luminous | Element |
|---|---|---|---|
| manufactured | Hole transport | efficiency | service life |
| elements | layer material | @10 mA/cm² | LT50 |
| Example 1-1 | Example Compound | 152% | 178% |
| | A10 | | |
| Example 1-2 | Example Compound | 150% | 180% |
| | A110 | | |
| Example 1-3 | Example Compound | 154% | 172% |
| | A152 | | |
| Example 1-4 | Example Compound | 153% | 175% |
| | B11 | | |
| Example 1-5 | Example Compound | 149% | 177% |
| | B130 | | |
| Example 1-6 | Example Compound | 156% | 165% |
| | B163 | | |
| Example 1-7 | Example Compound | 150% | 168% |
| | B198 | | |
| Example 1-8 | Example Compound | 145% | 179% |
| | C1 | | |
| Example 1-9 | Example Compound | 148% | 182% |
| | C35 | | |
| Example 1-10 | Example Compound | 147% | 185% |
| | C47 | | |
| Example 1-11 | Example Compound | 145% | 180% |
| | C90 | | |
| Example 1-12 | Example Compound | 146% | 174% |
| | C175 | | |
| Example 1-13 | Example Compound | 147% | 178% |
| | C214 | | |
| Example 1-14 | Example Compound | 152% | 174% |
| | C293 | | |
| Example 1-15 | Example Compound | 149% | 170% |
| | C328 | | |
| Example 1-16 | Example Compound | 153% | 169% |
| | D16 | | |
| Example 1-17 | Example Compound | 148% | 176% |
| | D60 | | |
| Comparative | Comparative Example | 86% | 88% |
| Example 1-1 | Compound R1 | | |
| Comparative | Comparative Example | 94% | 92% |
| Example 1-2 | Compound R2 | | |
| Comparative | Comparative Example | 100% | 100% |
| Example 1-3 | Compound R3 | | |
| Comparative | Comparative Example | 105% | 85% |
| Example 1-4 | Compound R4 | | |
| Comparative | Comparative Example | 102% | 94% |
| Example 1-5 | Compound R5 | | |
| Comparative | Comparative Example | 103% | 110% |
| Example 1-6 | Compound R6 | | |
| Comparative | Comparative Example | 101% | 105% |
| Example 1-7 | Compound R7 | | |
| Comparative | Comparative Example | 87% | 77% |
| Example 1-8 | Compound R8 | | |

Referring to the results of Table 1, it may be seen that Examples of the light emitting elements using the amine compounds of examples according to the present disclosure as a hole transport layer material exhibit excellent (suitable) luminous efficiency and improved service life characteristics. For example, referring to Table 1, it may be confirmed that the light emitting elements of Examples 1-1 to 1-17 exhibit long service lives and high efficiency characteristics compared with those of Comparative Examples 1-1 to 1-8. For example, it may be confirmed that the amine compounds of examples include the benzocarbazole moiety and at least one of a naphthyl group, a phenanthryl group, a benzoheterole group, or a fluorenyl group, thereby exhibiting high efficiency and long service life characteristics at the same time (concurrently) compared with Comparative Examples.

For example, the amine compound of an example including the benzocarbazole moiety in which an aromatic ring is fused to a 2-carbazole skeleton includes a structure in which an aromatic ring is fused at the position in which HOMO orbital extends, thereby exhibiting improved stability of radical or radical cation state. In some embodiments, the aromatic ring extends towards the outside of the molecule, and thus the intermolecular π-π interaction may be enhanced, and the hole transport property may be improved, which may contribute to reduced driving voltage and high efficiency. In some embodiments, the amine compound of an example may exhibit improved electron resistance and exciton resistance by introducing, into the amine moiety, a substituent such as a naphthyl group, a phenanthryl group, a benzoheterole group, or a fluorenyl group. It is confirmed that these effects are synergistic, and thus the amine compound of an example may be used as a material for the light emitting element, thereby achieving high efficiency and long service life characteristics.

In comparison, Comparative Example Compound R1 used in Comparative Example 1-1 has a carbazole skeleton, which has a decrease in stabilizing effect and effect of improving the hole transport property by the benzocarbazole skeleton of the amine compound of an example, and thus both luminous efficiency and element service life are reduced compared with Example Compounds.

Comparative Example Compound R2 used in Comparative Example 1-2 has the benzocarbazole skeleton, but the benzocarbazole is directly bonded to the amine, leading to narrow extending of the HOMO orbital, and thus the element service life is reduced compared with Example Compounds.

Comparative Example Compound R3 used in Comparative Example 1-3 has the benzocarbazole skeleton, but does not have, at the amine side, substituents such as Formula 2 to Formula 5 described in the present disclosure, and the electron resistance and exciton resistance are not sufficient (suitable), and thus both luminous efficiency and element service life are reduced compared with Example Compounds.

Comparative Example Compounds R4 and R5 used in Comparative Examples 1-4 and 1-5 have a different linker, which links the benzocarbazole and the amine, from Example Compounds, and the linker is twisted, leading to a narrow extending of the HOMO orbital, and thus the element service life is reduced compared with Example Compounds.

It may be seen that Comparative Example Compounds R6 and R7 used in Comparative Examples 1-6 and 1-7 have an aryl group having many (plural) carbon atoms that is substituted at the nitrogen atom of the benzocarbazole, and thus both luminous efficiency and element service life are reduced compared with Example

Compounds. When the aryl group having many (plural) carbon atoms is substituted at the nitrogen atom of the benzocarbazole, it is supposed (postulated) that the deposition temperature of material is elevated, the bond between the corresponding N-aryl is cleaved, and thus the material deteriorates. Similar to the Example Compounds, a phenyl group may be substituted at the nitrogen atom of the benzocarbazole, thereby suppressing the elevation of the deposition temperature and exhibiting excellent (suitable) element characteristics.

Comparative Example Compound R8 used in Comparative Example 1-8 has an aryl group having many (plural) carbon atoms that is bonded to the nitrogen atom of the benzocarbazole, and it may be seen that the electron resistance and exciton resistance of the dihydrophenanthrene moiety are not sufficient (suitable), and thus both luminous efficiency and element service life are reduced compared with Example Compounds.

TABLE 2

| Examples of | | Luminous | Element |
|---|---|---|---|
| manufactured | Electron blocking | efficiency | service life |
| elements | layer material | @10 mA/cm² | LT50 |
| Example 2-1 | Example Compound | 155% | 180% |
| | A10 | | |
| Example 2-2 | Example Compound | 153% | 185% |
| | A110 | | |
| Example 2-3 | Example Compound | 154% | 175% |
| | A152 | | |
| Example 2-4 | Example Compound | 151% | 179% |
| | B11 | | |
| Example 2-5 | Example Compound | 152% | 181% |
| | B130 | | |
| Example 2-6 | Example Compound | 155% | 169% |
| | B163 | | |
| Example 2-7 | Example Compound | 151% | 171% |
| | B198 | | |
| Example 2-8 | Example Compound | 144% | 179% |
| | C1 | | |
| Example 2-9 | Example Compound | 147% | 180% |
| | C35 | | |
| Example 2-10 | Example Compound | 149% | 190% |
| | C47 | | |
| Example 2-11 | Example Compound | 141% | 184% |
| | C90 | | |
| Example 2-12 | Example Compound | 148% | 173% |
| | C175 | | |
| Example 2-13 | Example Compound | 150% | 178% |
| | C214 | | |
| Example 2-14 | Example Compound | 154% | 170% |
| | C293 | | |
| Example 2-15 | Example Compound | 155% | 174% |
| | C328 | | |
| Example 2-16 | Example Compound | 156% | 167% |
| | D16 | | |
| Example 2-17 | Example Compound | 151% | 174% |
| | D60 | | |
| Comparative | Comparative Example | 85% | 90% |
| Example 2-1 | Compound R1 | | |
| Comparative | Comparative Example | 92% | 87% |
| Example 2-2 | Compound R2 | | |
| Comparative | Comparative Example | 100% | 100% |
| Example 2-3 | Compound R3 | | |
| Comparative | Comparative Example | 106% | 90% |
| Example 2-4 | Compound R4 | | |
| Comparative | Comparative Example | 103% | 89% |
| Example 2-5 | Compound R5 | | |
| Comparative | Comparative Example | 101% | 115% |
| Example 2-6 | Compound R6 | | |
| Comparative | Comparative Example | 98% | 109% |
| Example 2-7 | Compound R7 | | |
| Comparative | Comparative Example | 88% | 80% |
| Example 2-8 | Compound R8 | | |

Referring to the results of Table 2, it may be confirmed that the light emitting elements of Examples 2-1 to 2-17 exhibit long service lives and high efficiency characteristics compared with those of Comparative Examples 2-1 to 2-8. For example, it may be seen that even when the amine compound of an example is used in the electron blocking layer, the light emitting element may exhibit excellent (suitable) element characteristics.

Thus, the compounds used in Examples may improve luminous efficiency and luminous service life at the same time (concurrently) compared with the compound used in Comparative Examples. For example, the amine compound including the benzocarbazole moiety and at least one substituent of a naphthyl group, a phenanthryl group, a benzoheterole group, or a fluorenyl group is used in the light emitting element according to an embodiment, and thus it is possible to improve the efficiency and service life of the element at the same time.

The light emitting element of an embodiment may include the amine compound of an embodiment, thereby exhibiting high efficiency and long service life characteristics.

The amine compound of an embodiment may be used to achieve improved characteristics of the light emitting element having high efficiency and a long service life.

The use of “may” when describing embodiments of the present disclosure refers to “one or more embodiments of the present disclosure.”

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this disclosure is intended to include all higher numerical limitations subsumed therein. Accordingly, Applicant reserves the right to amend this disclosure, including the claims, to expressly recite any sub-range subsumed within the ranges expressly recited herein.

The light emitting element (or device) or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the present disclosure has been described with reference to preferred embodiments of the present disclosure, it will be understood that the present disclosure should not be limited to these preferred embodiments but one or more suitable changes and modifications can be made by those skilled in the art without departing from the spirit and scope of the present disclosure as defined by the following claims and equivalents thereof.

Accordingly, the technical scope of the present disclosure is not intended to be limited to the contents set forth in the detailed description of the disclosure, but is intended to be defined by the appended claims and equivalents thereof.

What is claimed is:

1. A light emitting element comprising:
a first electrode;
a second electrode on the first electrode; and
at least one functional layer which is between the first electrode and the second electrode and comprises an amine compound represented by Formula 1: 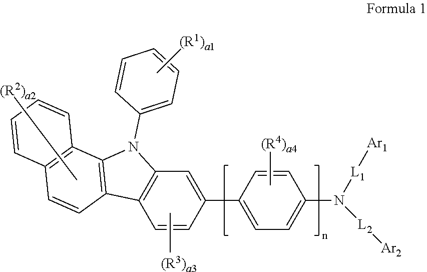
wherein, in Formula 1,
R¹ to R⁴ are each independently a hydrogen atom or a deuterium atom,
a1 is an integer from 0 to 5, a2 is an integer from 0 to 6, a3 is an integer from 0 to 3, a4 is an integer from 0 to 4, and n is 1 or 2,
L₁ and L₂ are each independently a direct linkage, a substituted or unsubstituted arylene group having 6 to 30 ring-forming carbon atoms, or a substituted or unsubstituted heteroarylene group having 5 to 30 ring-forming carbon atoms,
Ar₁ and Ar₂ are each independently a substituted or unsubstituted aryl group having 6 to 40 ring-forming carbon atoms, or a substituted or unsubstituted heteroaryl group having 5 to 40 ring-forming carbon atoms, or any one among Formula 2 to Formula 5, and
at least one of Ar₁ or Ar₂ is any one among Formula 2 to Formula 5: 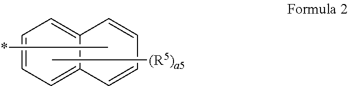 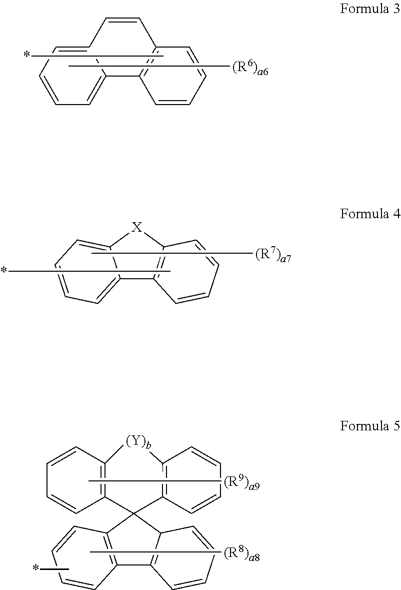
wherein, in Formula 2 to Formula 5, [symbol]
is a part bonded to L₁ or L₂ in Formula 1,
R⁵ to R⁹ are each independently a hydrogen atom, a deuterium atom, a halogen atom, a substituted or unsubstituted aryl group having 6 to 30 ring-forming carbon atoms, a substituted or unsubstituted heteroaryl group having 5 to 30 ring-forming carbon atoms, a substituted or unsubstituted alkyl group having 1 to 30 carbon atoms, or a substituted or unsubstituted alkenyl group having 2 to 30 carbon atoms, or are bonded to an adjacent group to form a ring,
a5, a7, and a8 are each independently an integer from 0 to 7, a6 is an integer from 0 to 9, and a9 is an integer from 0 to 8,
in Formula 4, X is O or S, and
in Formula 5, b is 0 or 1, and when b is 1, Y is a direct linkage.

2. The light emitting element of claim 1, wherein the at least one functional layer comprises an emission layer, a hole transport region between the first electrode and the emission layer, and an electron transport region between the emission layer and the second electrode, and
the hole transport region comprises the amine compound.

3. The light emitting element of claim 2, wherein the hole transport region comprises at least one selected from a hole injection layer, a hole transport layer, and an electron blocking layer, and
at least one selected from the hole injection layer, the hole transport layer, and the electron blocking layer comprises the amine compound.

4. The light emitting element of claim 1, wherein, in Formula 1,
when any one among Ar₁ and Ar₂ is Formula 2, the other is Formula 2 or Formula 3,
when any one among Ar₁ and Ar₂ is Formula 3, the other is a substituted or unsubstituted aryl group having 6 to 40 ring-forming carbon atoms, a substituted or unsubstituted heteroaryl group having 5 to 40 ring-forming carbon atoms, or any one among Formula 3 to Formula 5,
when any one among Ar₁ and Ar₂ above is Formula 4, the other is a substituted or unsubstituted aryl group having 6 to 40 ring-forming carbon atoms, a substituted or unsubstituted heteroaryl group having 5 to 40 ring-forming carbon atoms, or any one among Formula 4 and Formula 5, or
when any one among Ar₁ and Ar₂ is Formula 5, the other is a substituted or unsubstituted aryl group having 6 to 40 ring-forming carbon atoms, or a substituted or unsubstituted heteroaryl group having 5 to 40 ring-forming carbon atoms.

5. The light emitting element of claim 1, wherein Formula 1 is represented by Formula 1-1 below: 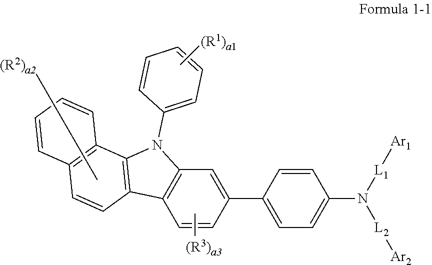
wherein, in Formula 1-1, R¹ to R³, a1 to a3, L₁, L₂, Ar₁, and Ar₂ are the same as defined in Formula 1.

6. The light emitting element of claim 1, wherein L₁ and L₂ are each independently a direct linkage, or a substituted or unsubstituted arylene group having 6 to 30 ring-forming carbon atoms.

7. The light emitting element of claim 1, wherein Formula 2 is represented by any one amona 2-1 and 2-2: 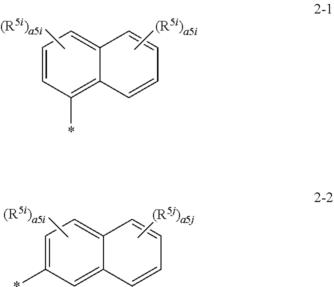
wherein, in 2-1 and 2-2,
R⁵ⁱ and R^{5j} are each independently a hydrogen atom, a deuterium atom, or a substituted or unsubstituted aryl group having 6 to 30 ring-forming carbon atoms, and
a5i is an integer from 0 to 3, and a5j is an integer from 0 to 4.

8. The light emitting element of claim 1, wherein Formula 3 is represented by any one among 3-1 to 3-3: 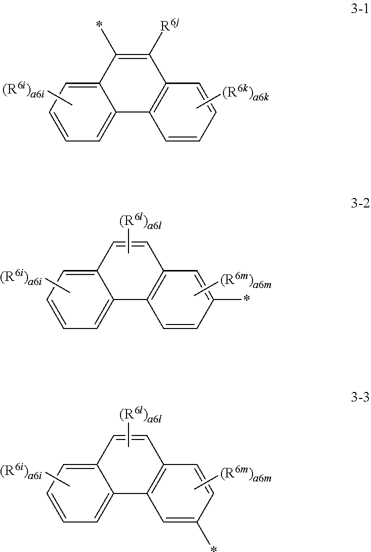
wherein, in 3-1 to 3-3,
R⁶ⁱ, R^{6j}, R^{6k}, R^{6l}, and R^{6m} are each independently a hydrogen atom, a deuterium atom, or a substituted or unsubstituted aryl group having 6 to 30 ring-forming carbon atoms,
a6i is an integer from 0 to 4,
in 3-1, a6k is an integer from 0 to 4, and
in 3-2 and 3-3, a61 is an integer from 0 to 2, and a6m is an integer from 0 to 3.

9. The light emitting element of claim 1, wherein Formula 4 is represented by any one among 4-1 to 4-4: 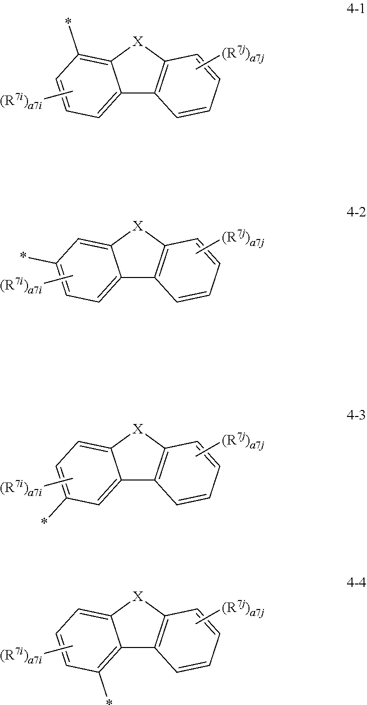
wherein, in 4-1 to 4-4,
R⁷ⁱ and R^{7j} are each independently a hydrogen atom, a deuterium atom, or a substituted or unsubstituted aryl group having 6 to 30 ring-forming carbon atoms, or are bonded to an adjacent group to form a ring, and
a7i is an integer from 0 to 3, and a7j is an integer from 0 to 4.

10. The light emitting element of claim 1, wherein Formula 5 is represented by any one among 5-1 to 5-6: 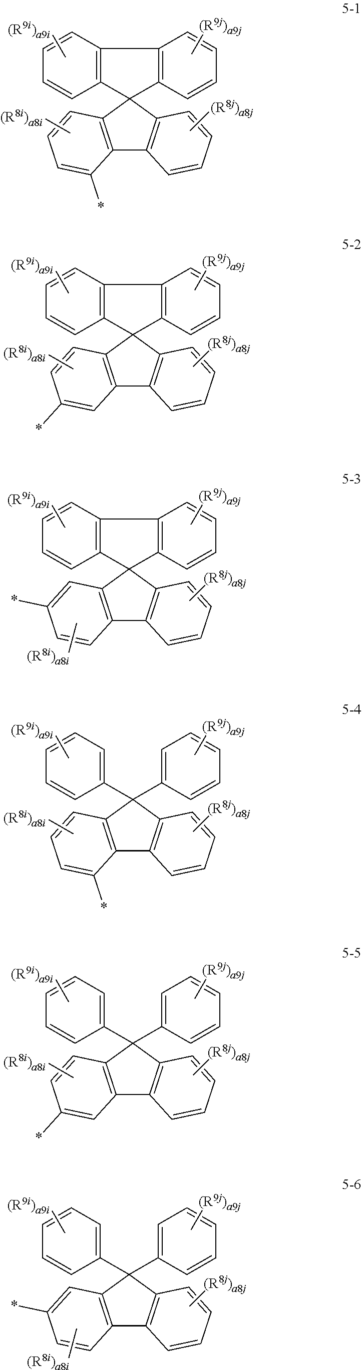
wherein, in 5-1 to 5-6,
R⁸ⁱ, R^{8j}, R⁹ⁱ, and R^{9j} are each independently a hydrogen atom, a deuterium atom, or a substituted or unsubstituted aryl group having 6 to 30 ring-forming carbon atoms, or are bonded to an adjacent group to form a ring,
a8i is an integer from 0 to 3, and a9i and a9i is an integer from 0 to 4.

11. The light emitting element of claim 2, wherein the emission layer comprises a compound represented by Formula E-1: 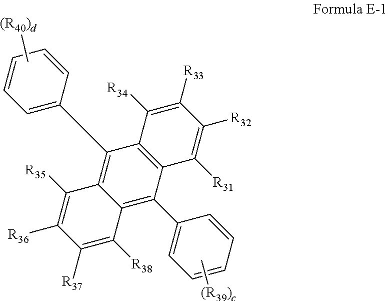
wherein, in Formula E-1, R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and
c and d are each independently an integer from 0 to 5.

12. The light emitting element of claim 1, wherein the amine compound is represented by any one among compounds of Compound Group 1:

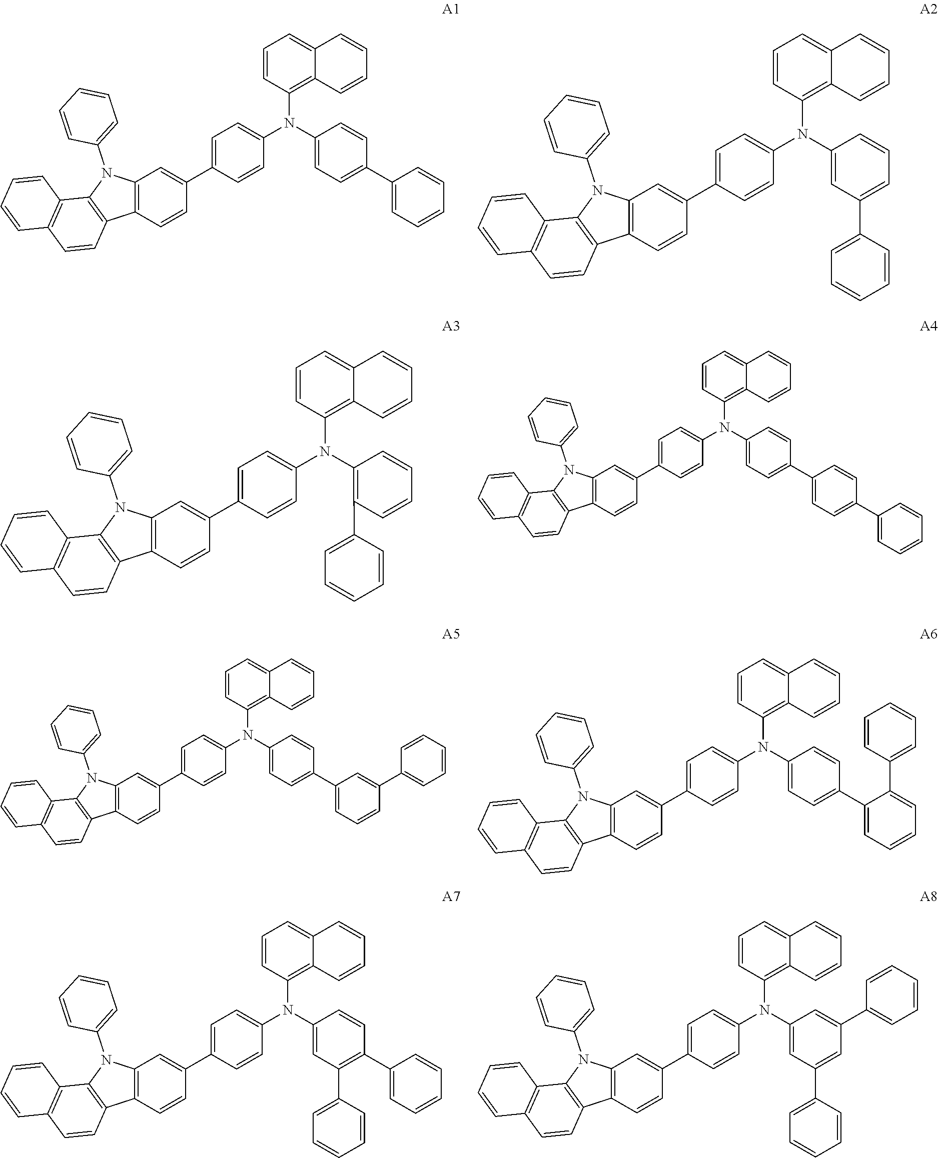

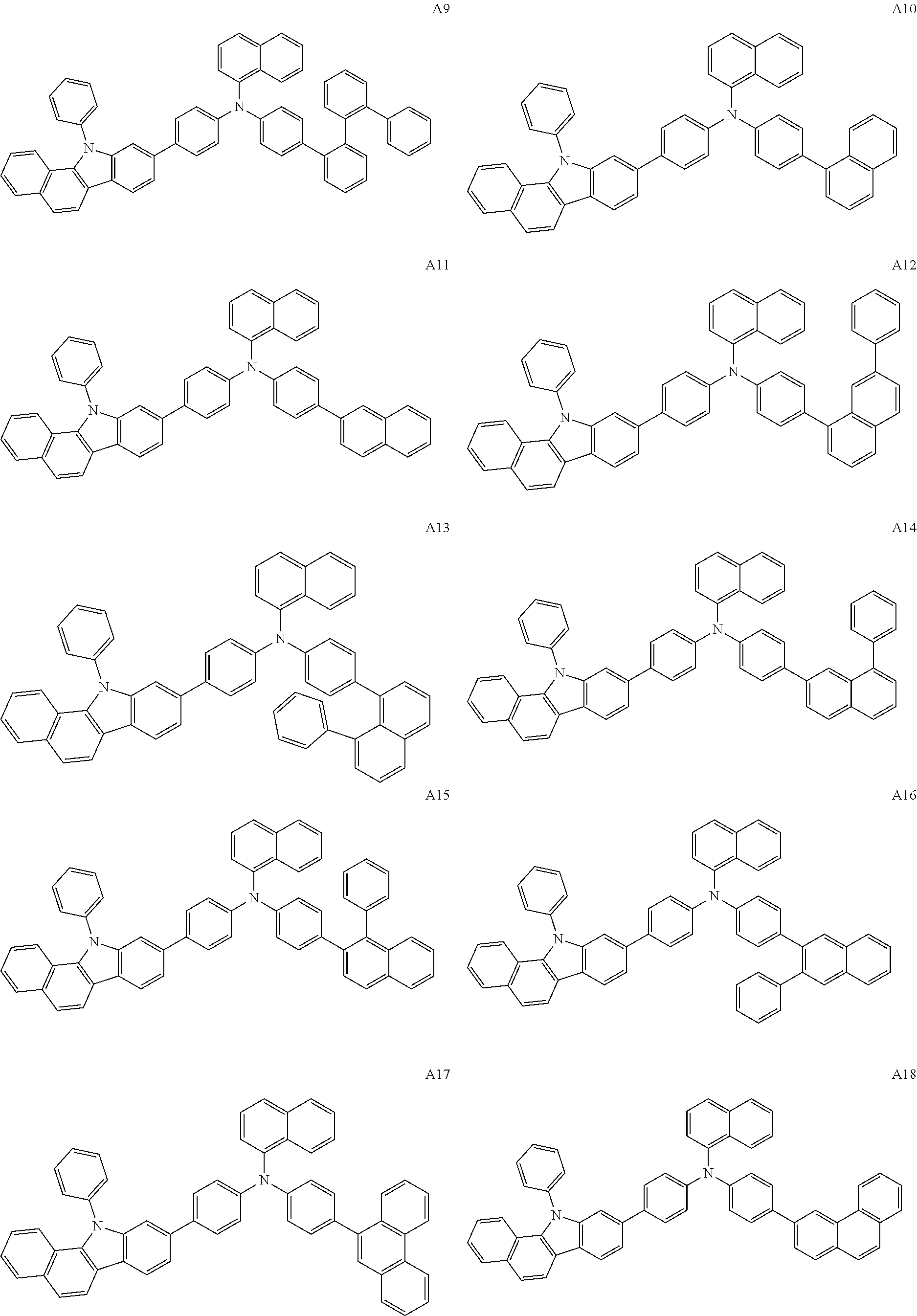

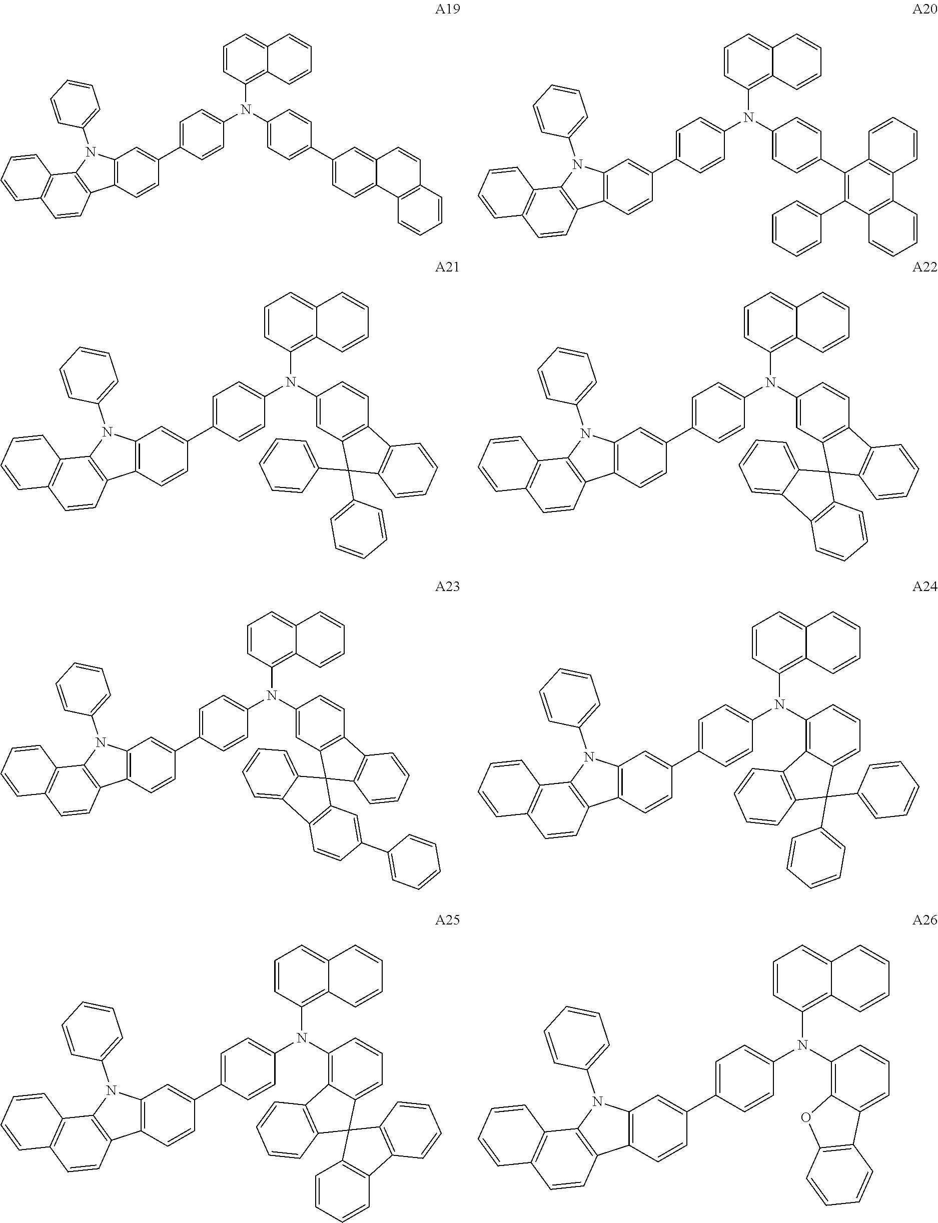

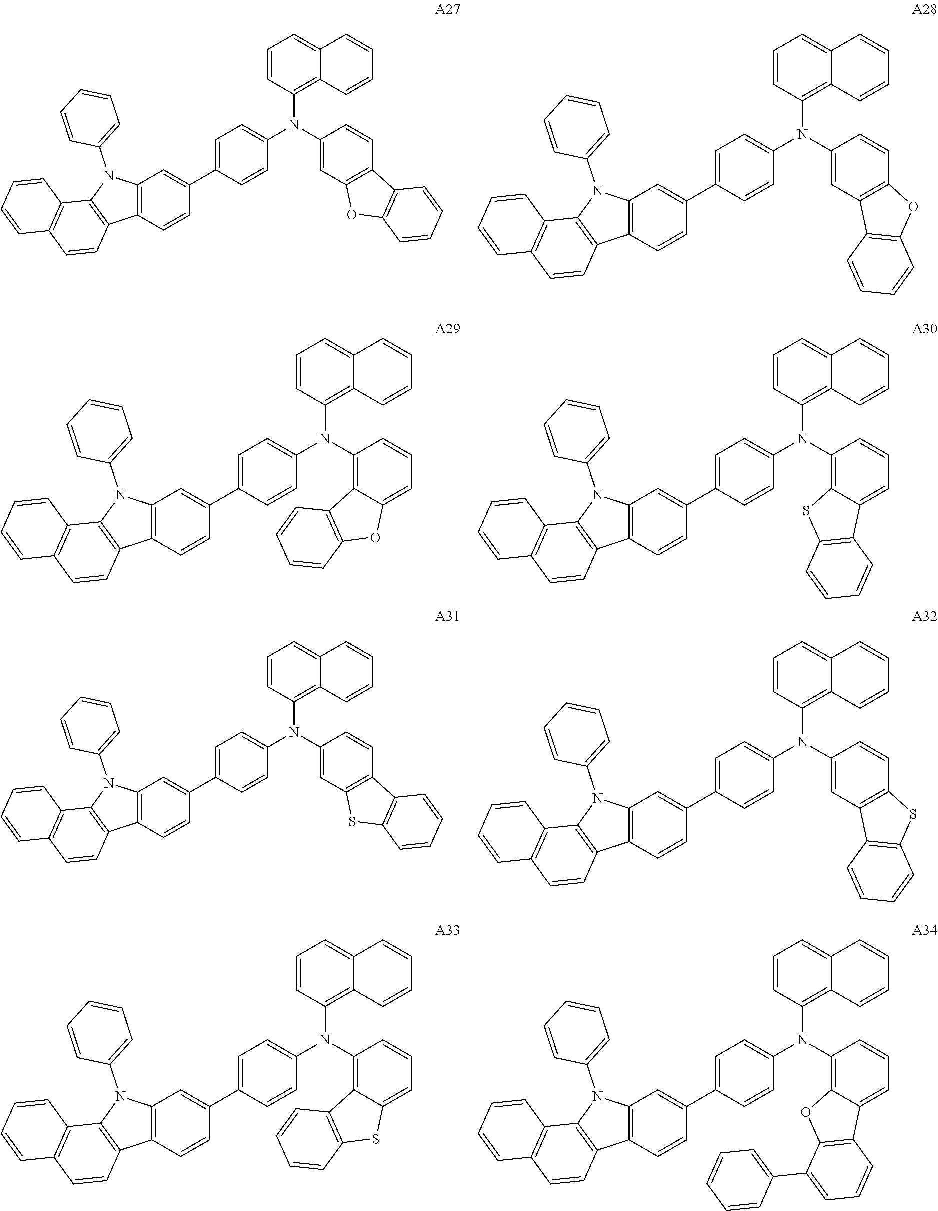

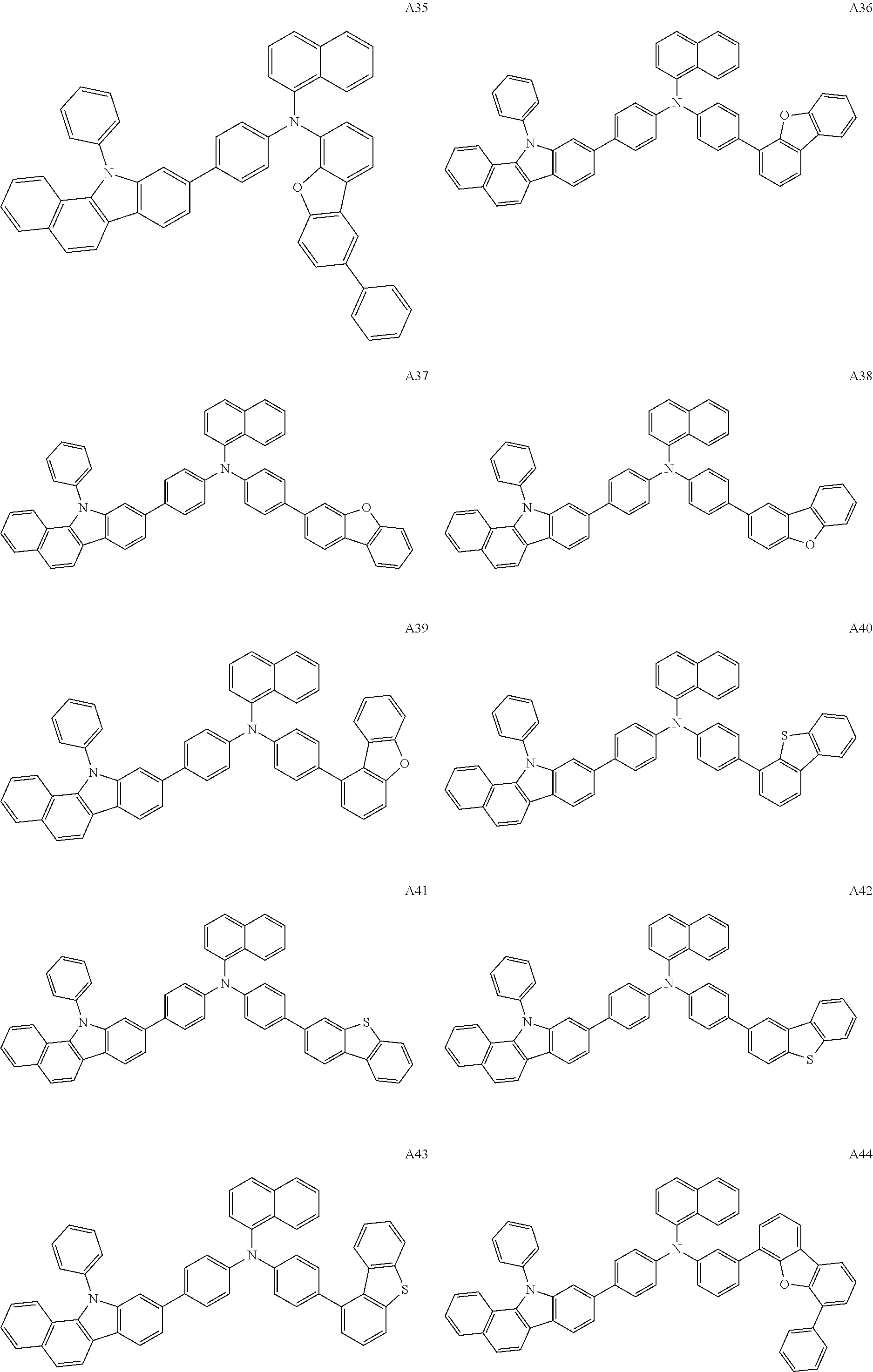

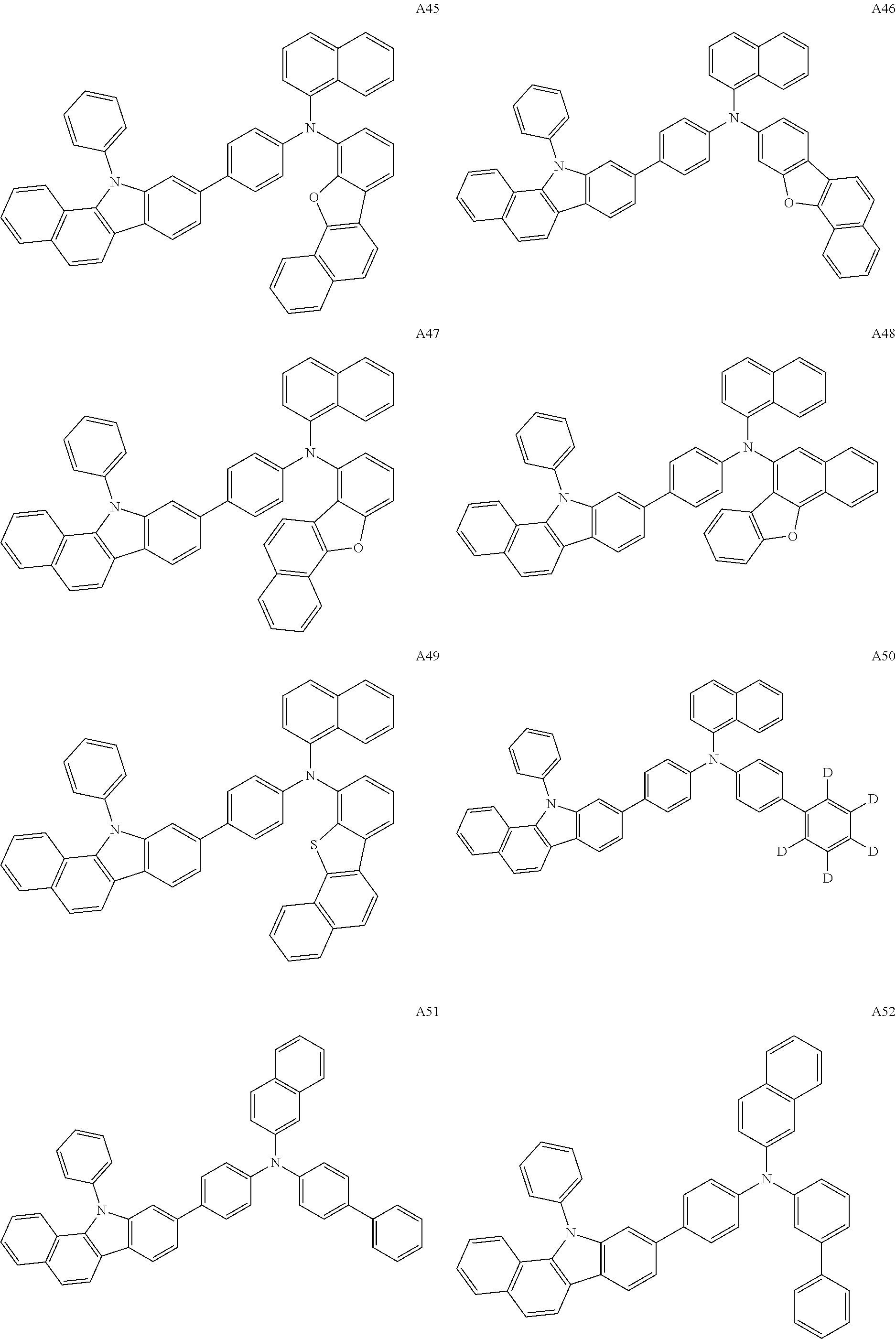

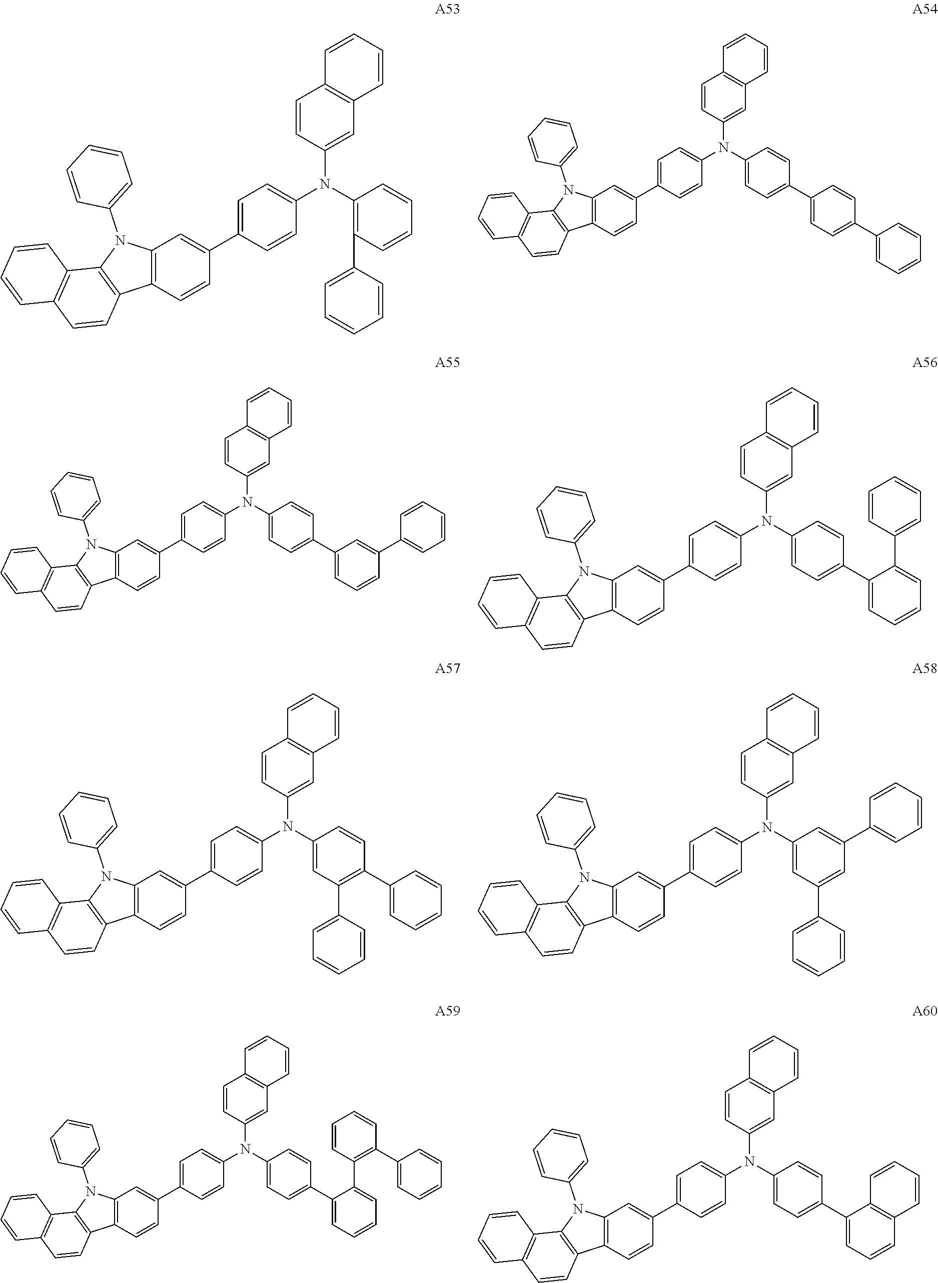

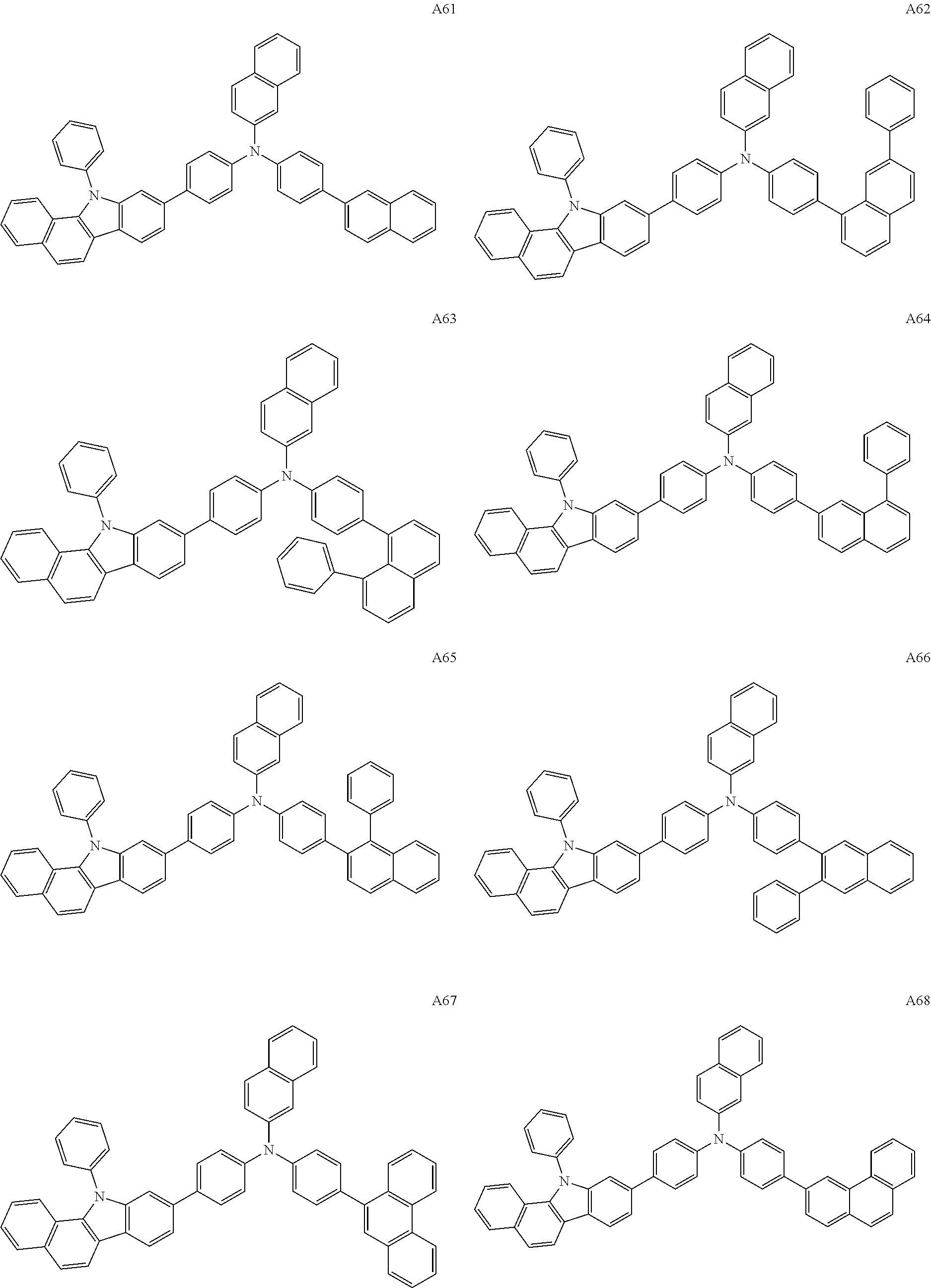

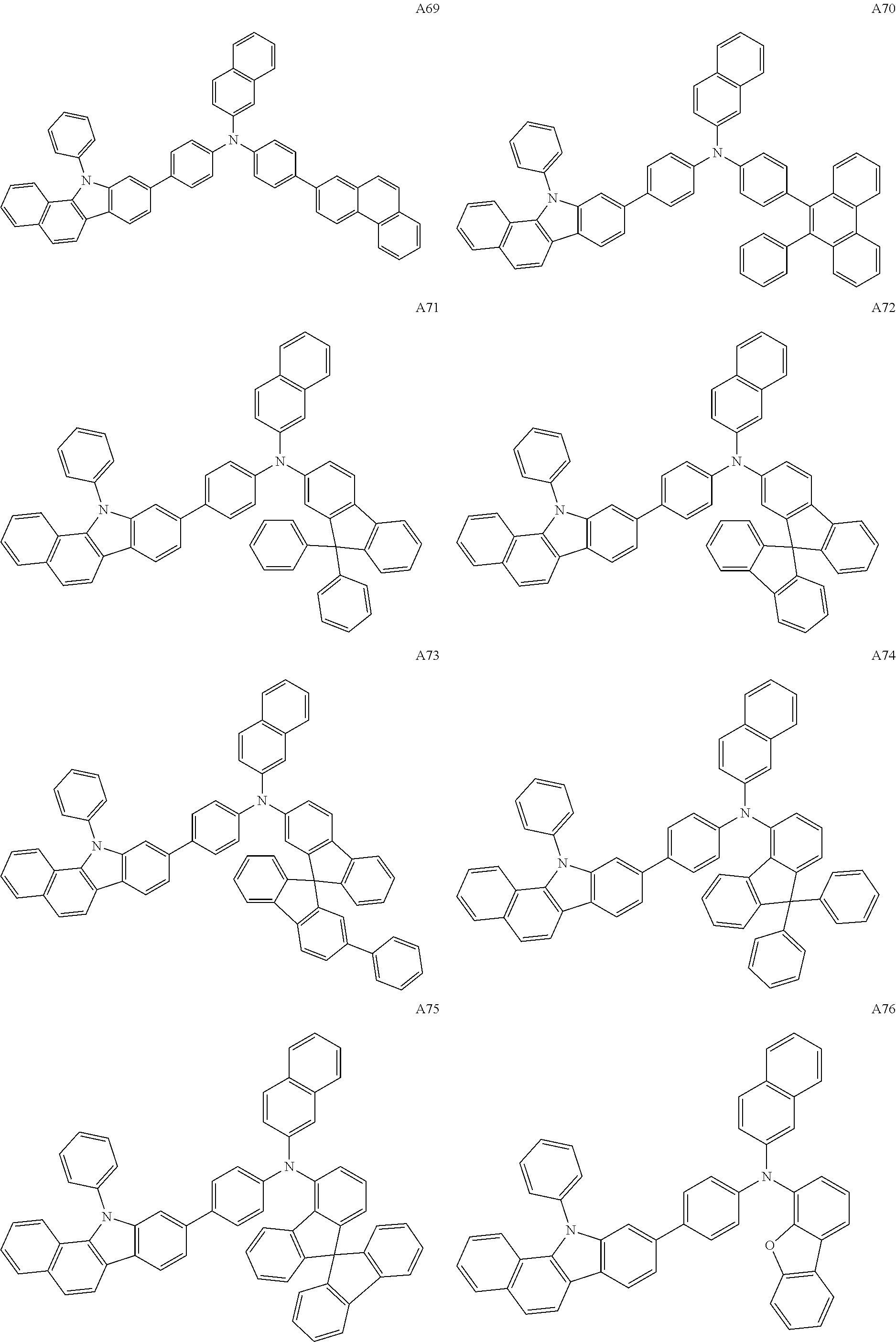

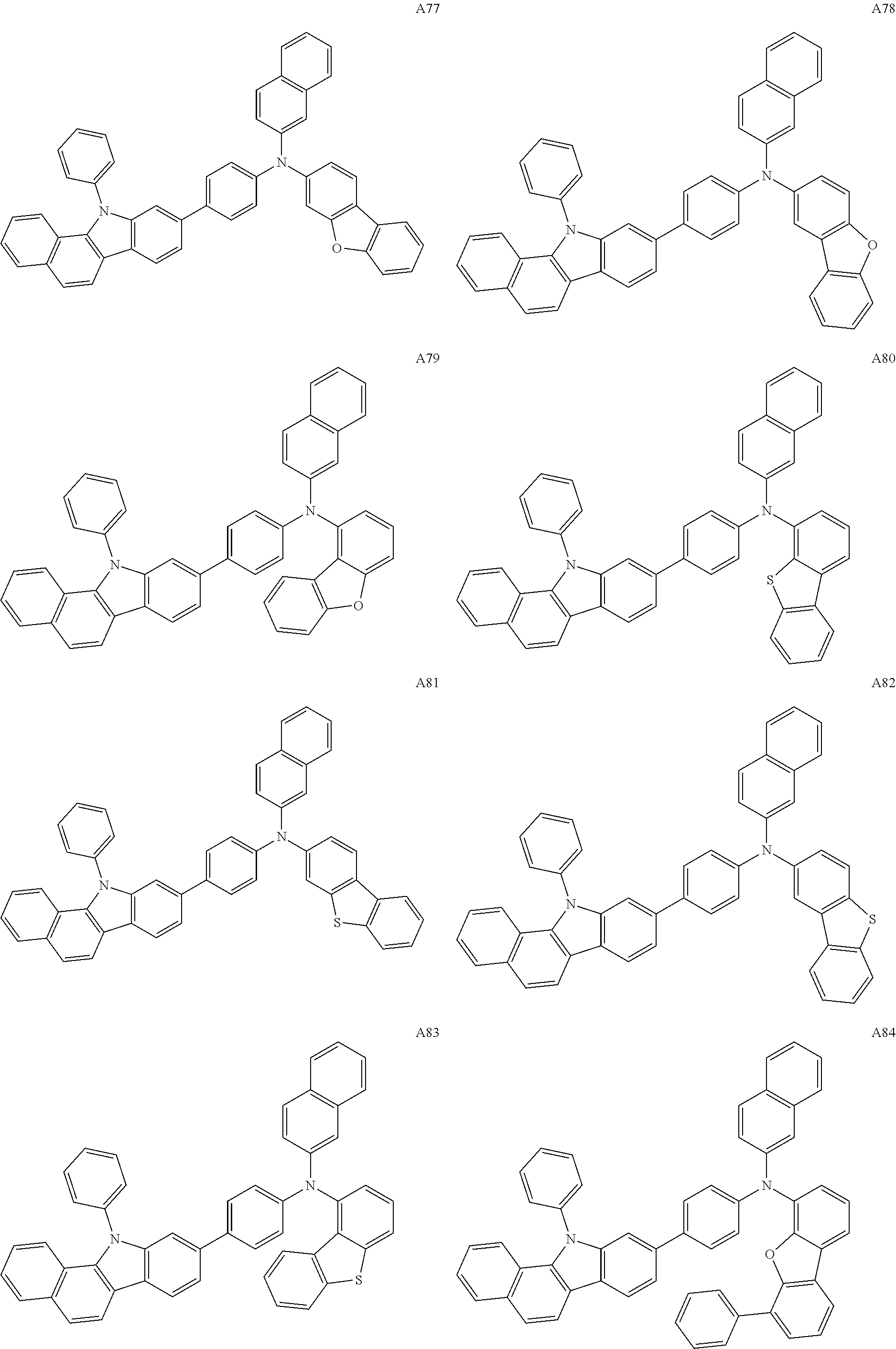

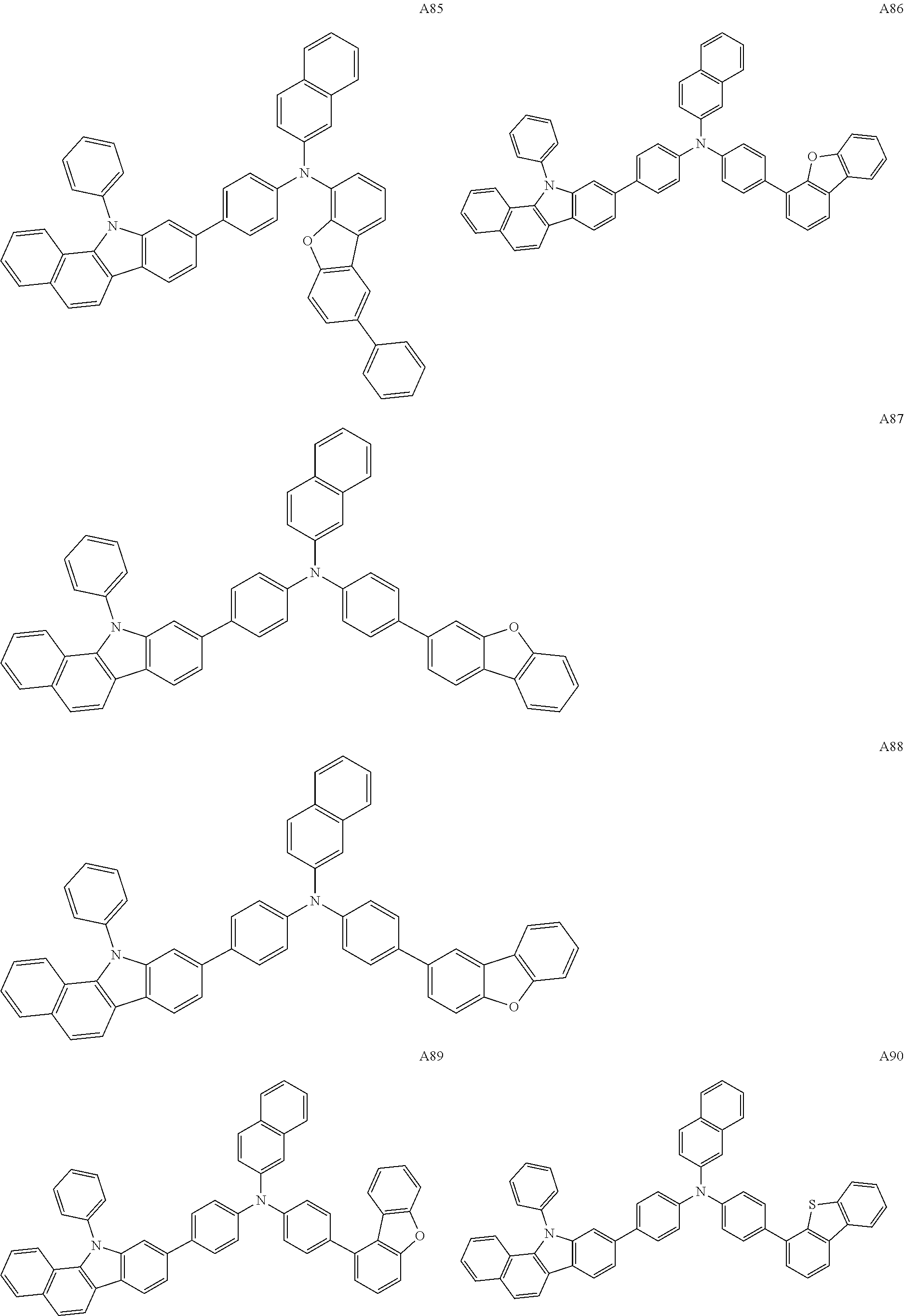

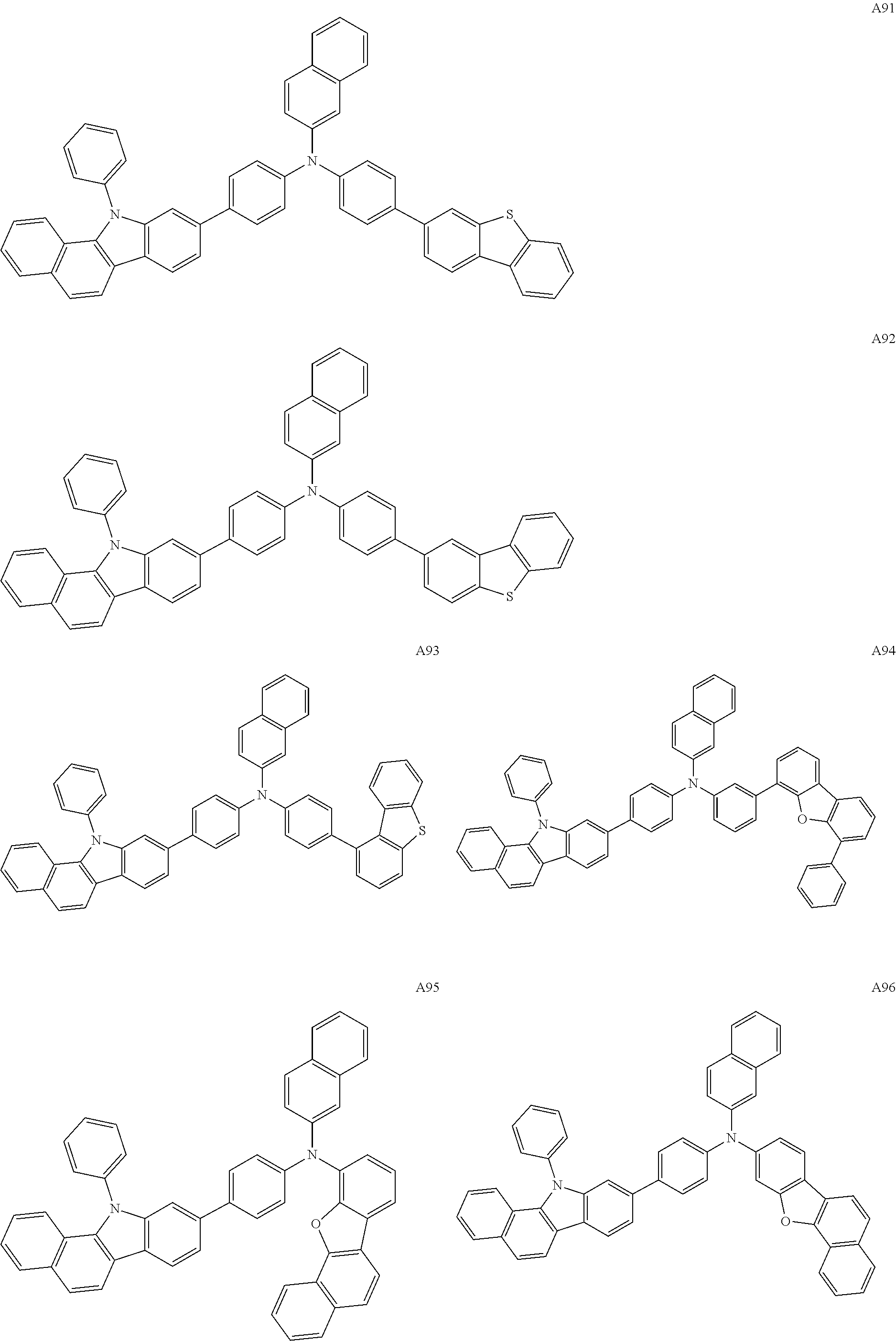

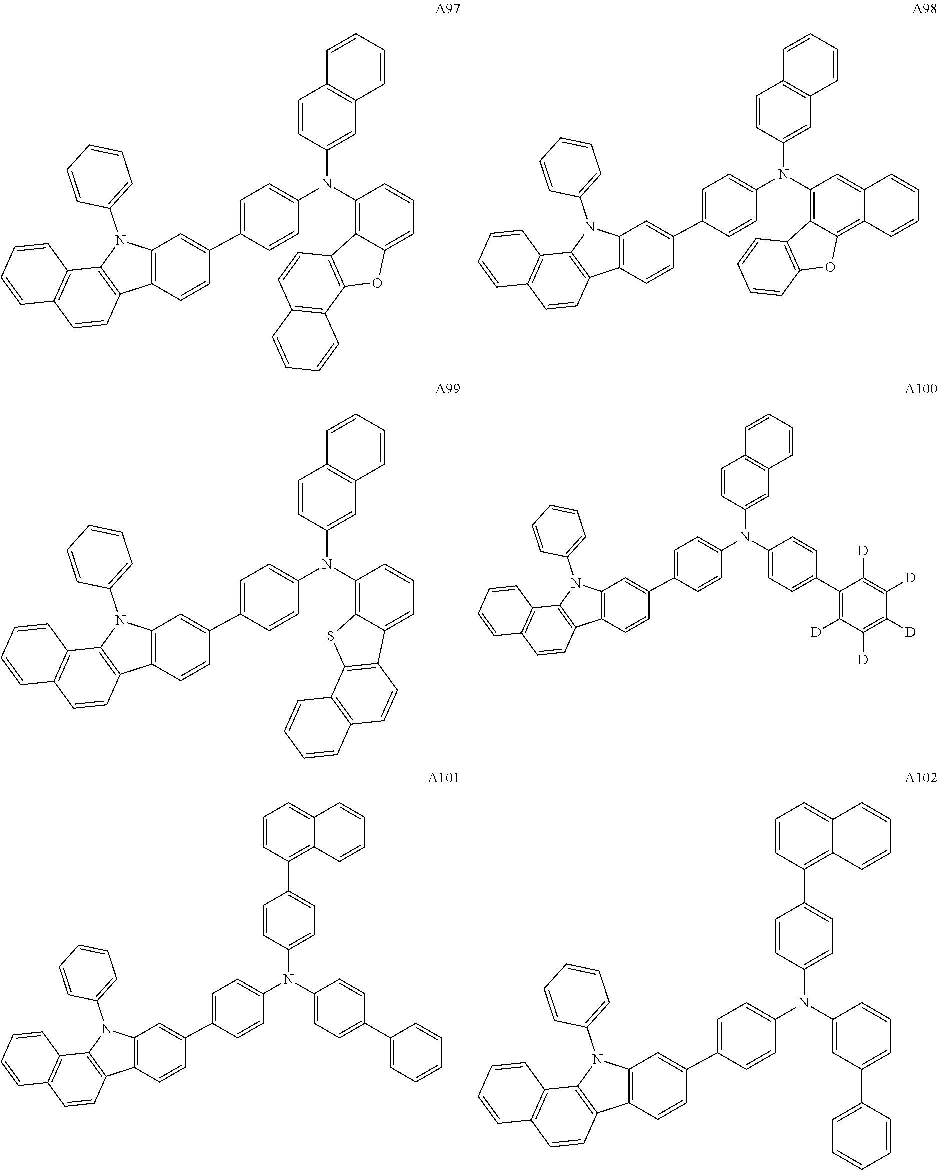

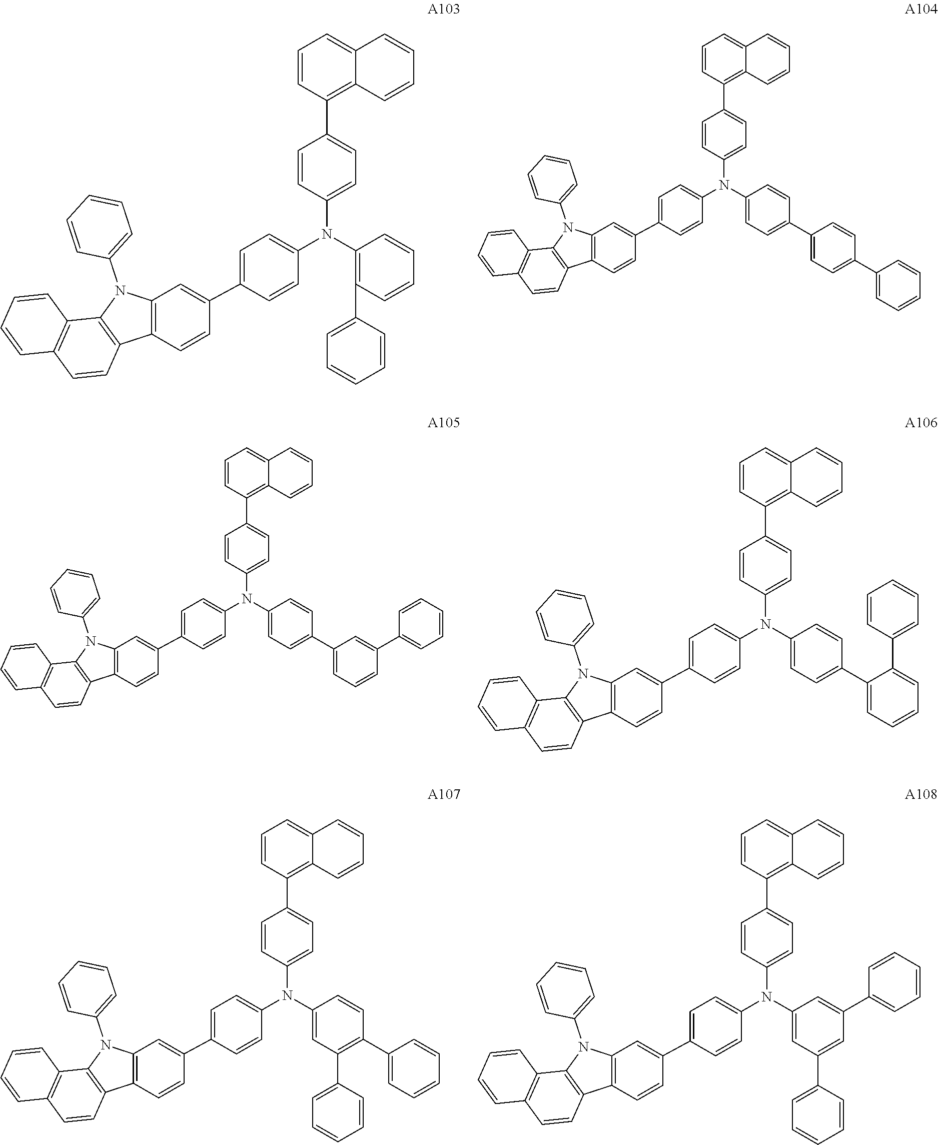

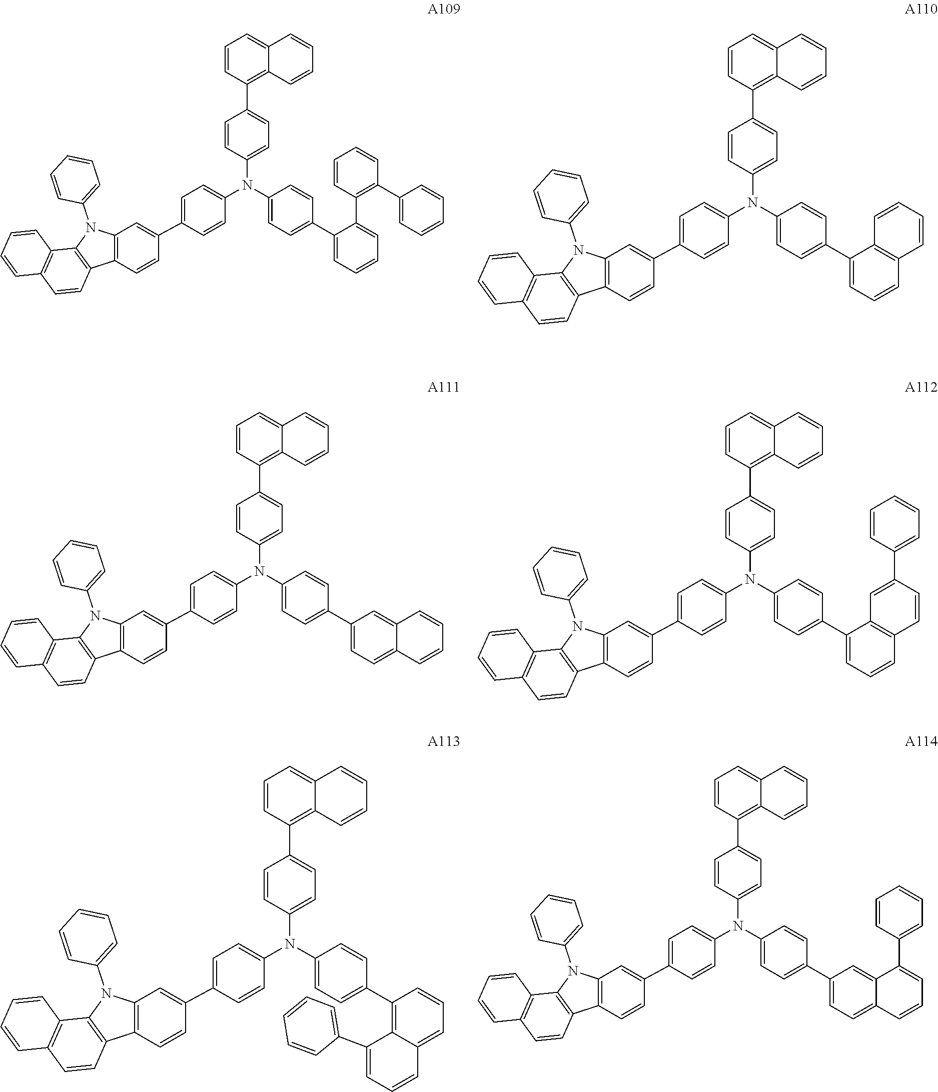

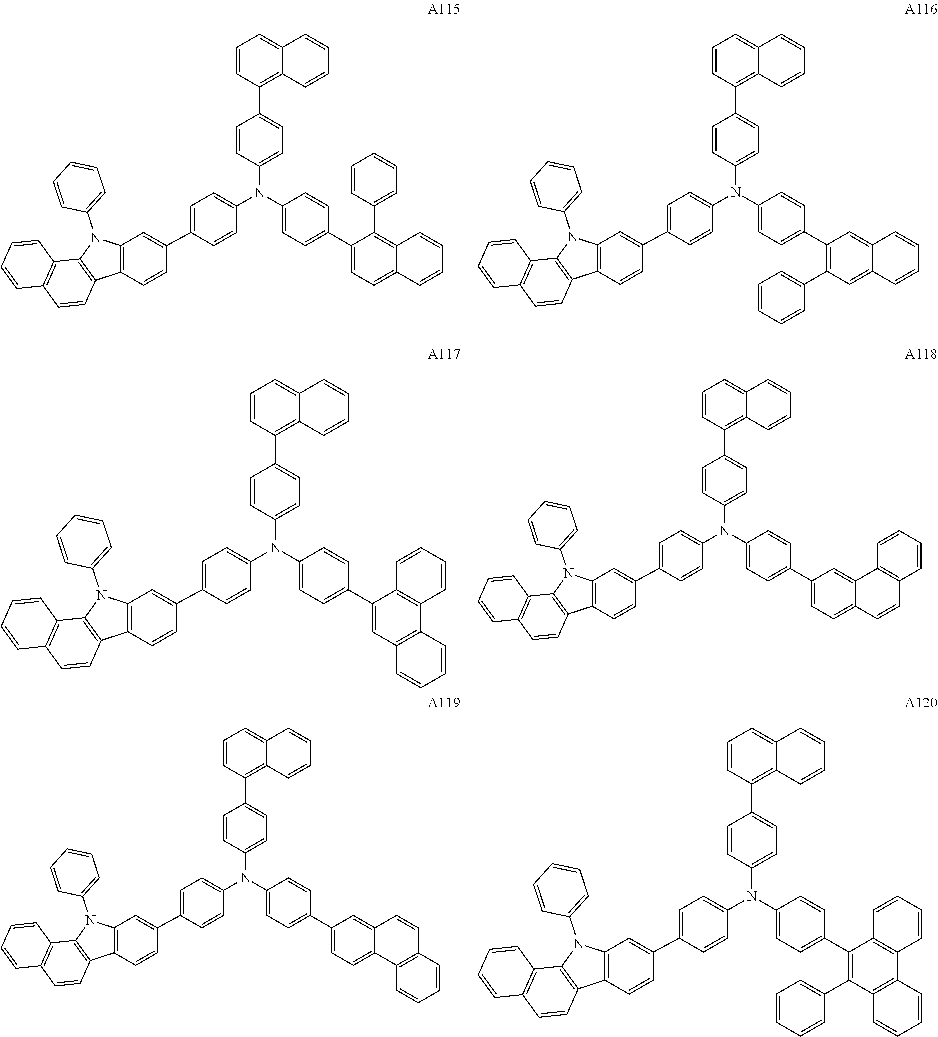

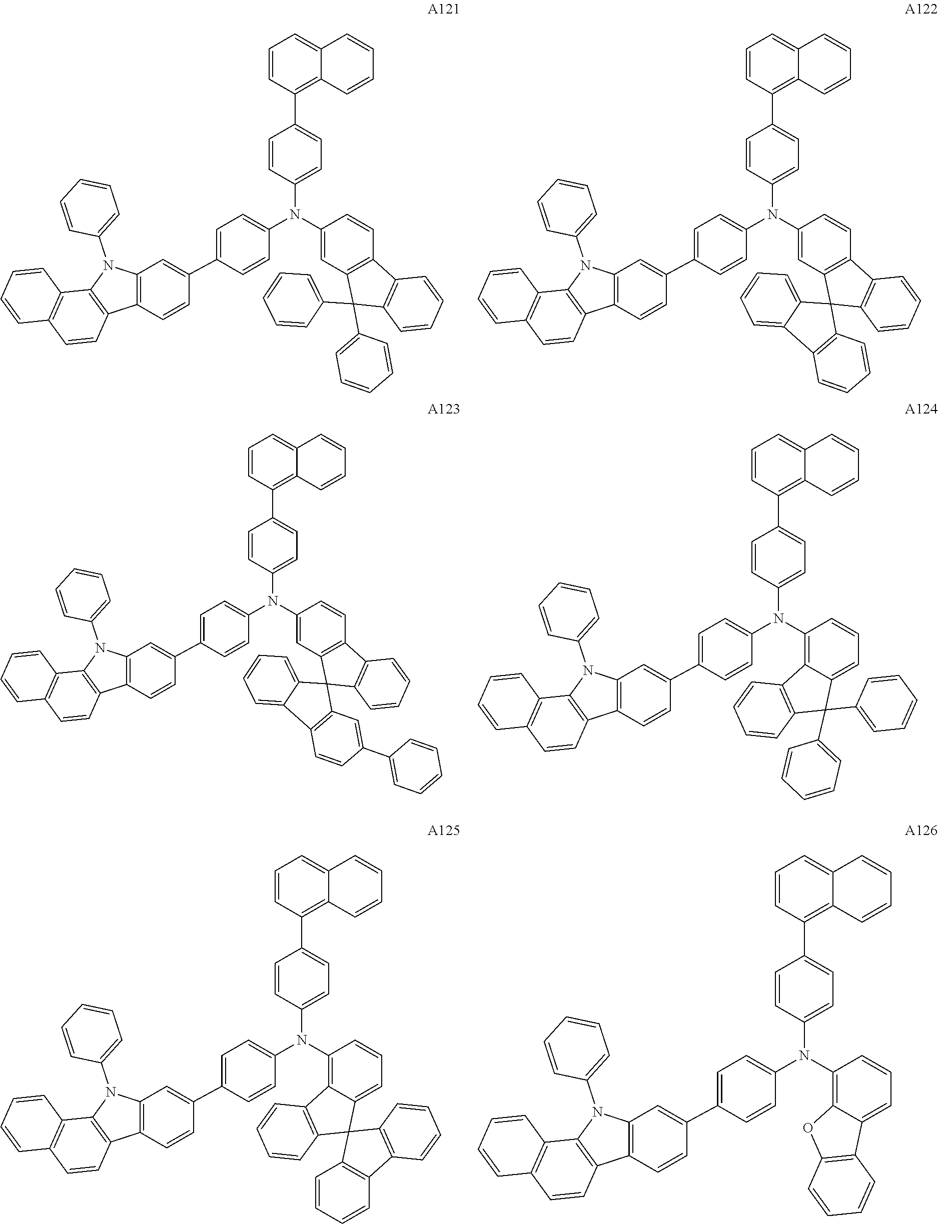

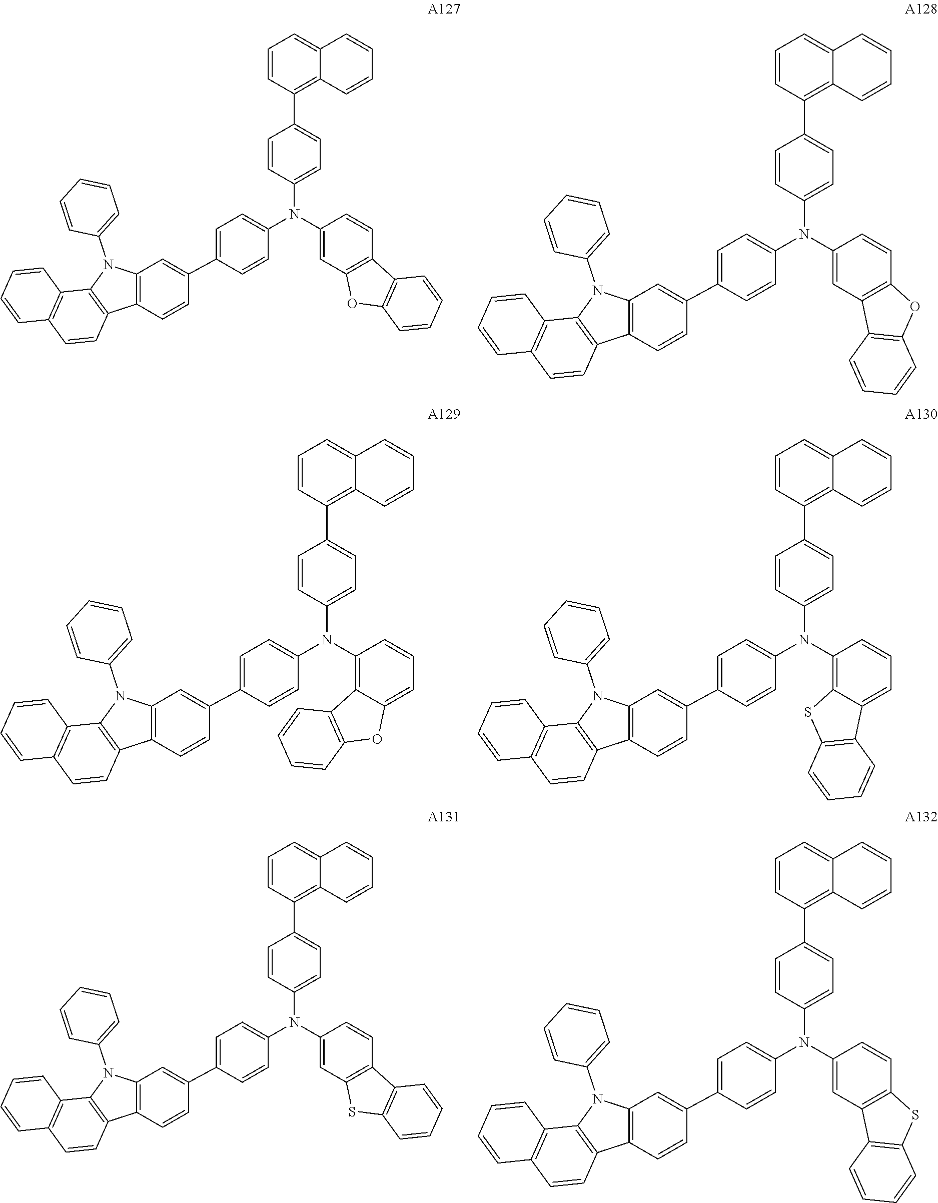

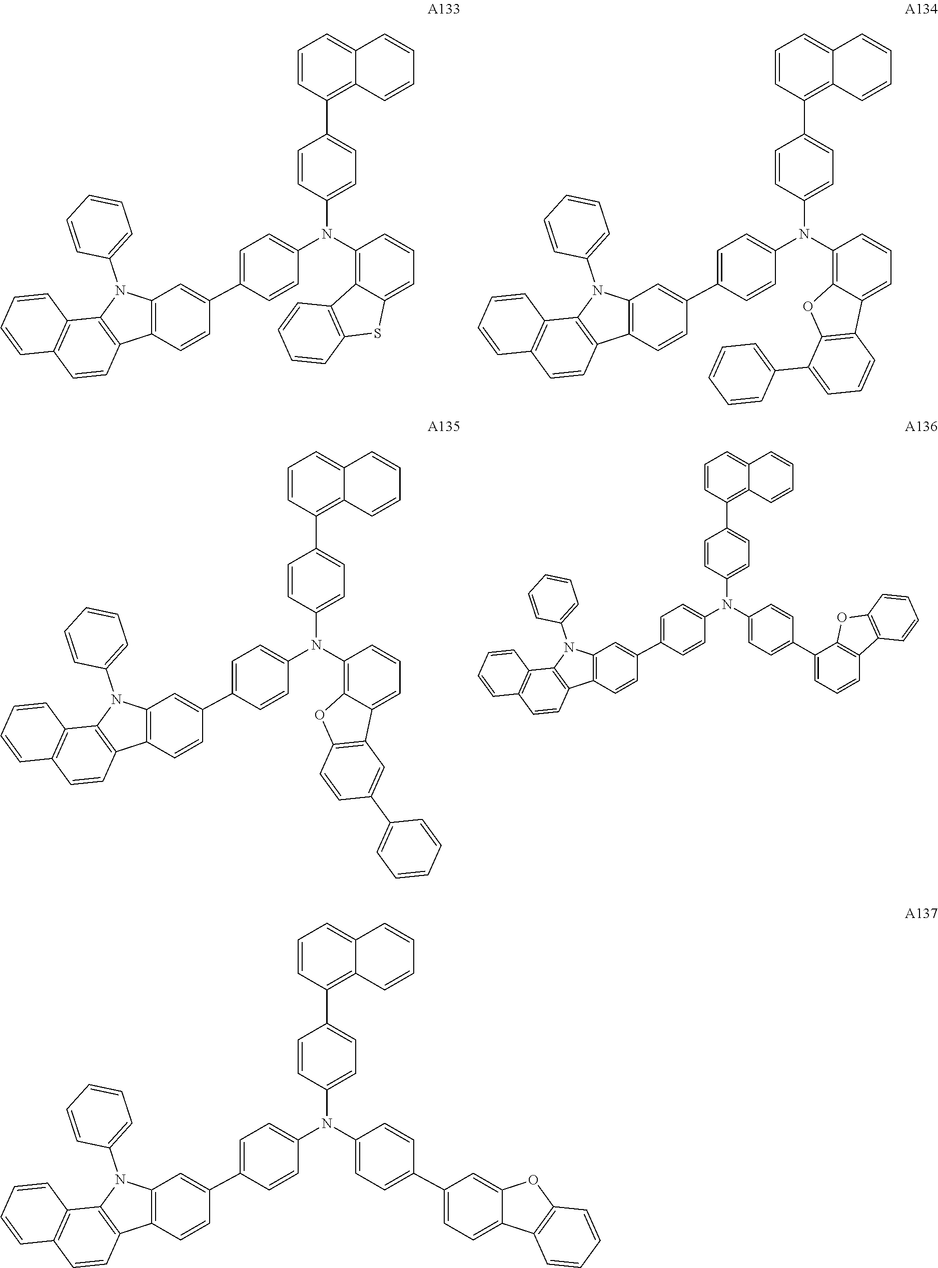

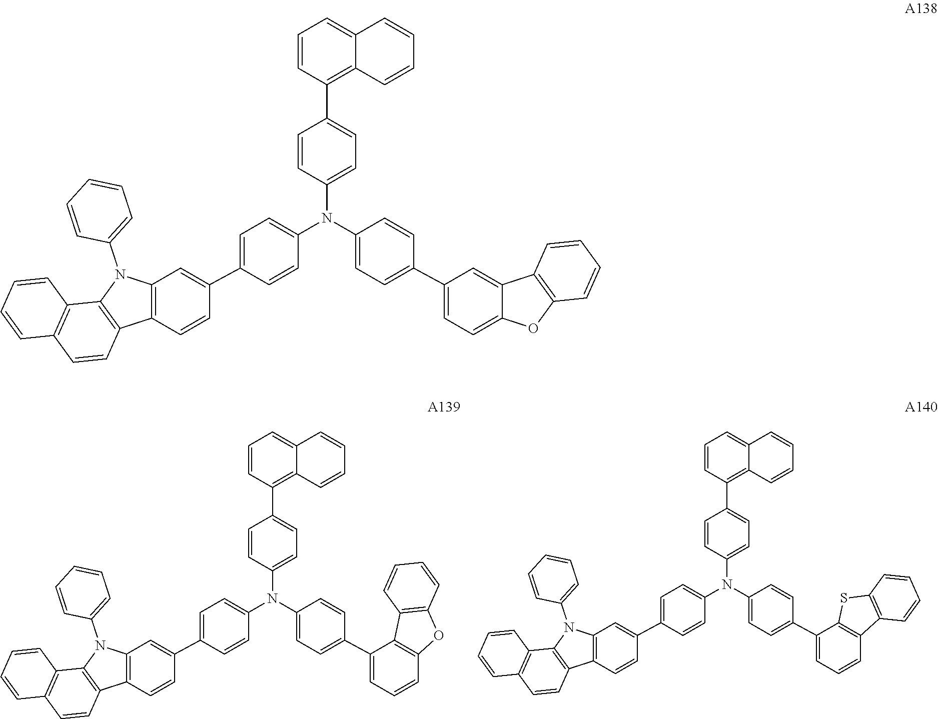 [SMILES: c1ccc(-n2c3cc(-c4ccc(N(c5ccc(-c6ccc7oc8ccccc8c7c6)cc5)c5ccc(-c6cccc7ccccc67)cc5)cc4)ccc3c3ccc4ccccc4c32)cc1 ; c1ccc(-n2c3cc(-c4ccc(N(c5ccc(-c6cccc7ccccc67)cc5)c5ccc(-c6cccc7oc8ccccc8c67)cc5)cc4)ccc3c3ccc4ccccc4c32)cc1 ; c1ccc(-n2c3cc(-c4ccc(N(c5ccc(-c6cccc7ccccc67)cc5)c5ccc(-c6cccc7c6sc6ccccc67)cc5)cc4)ccc3c3ccc4ccccc4c32)cc1]

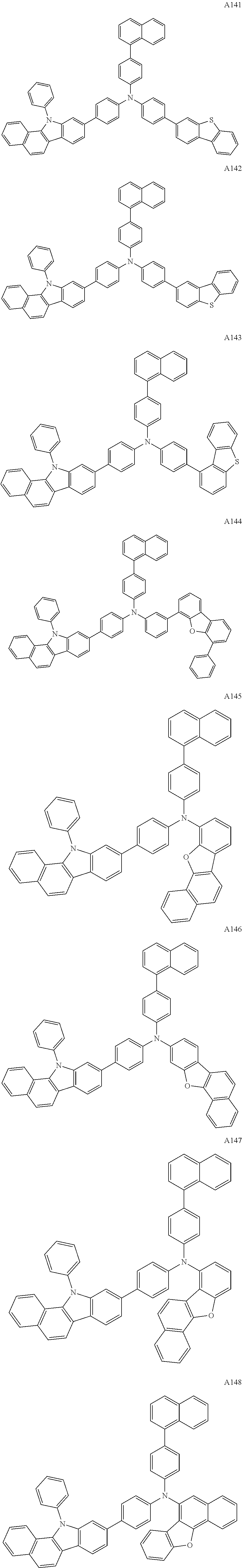

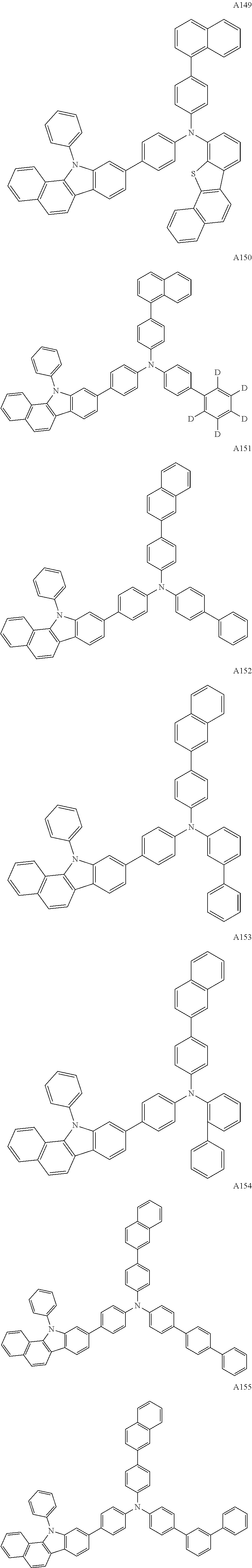

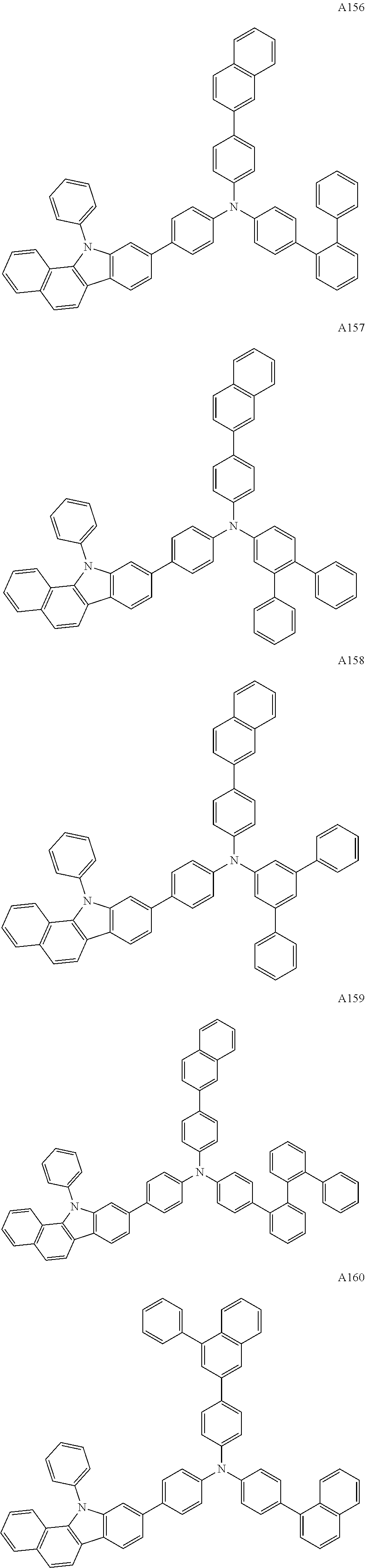

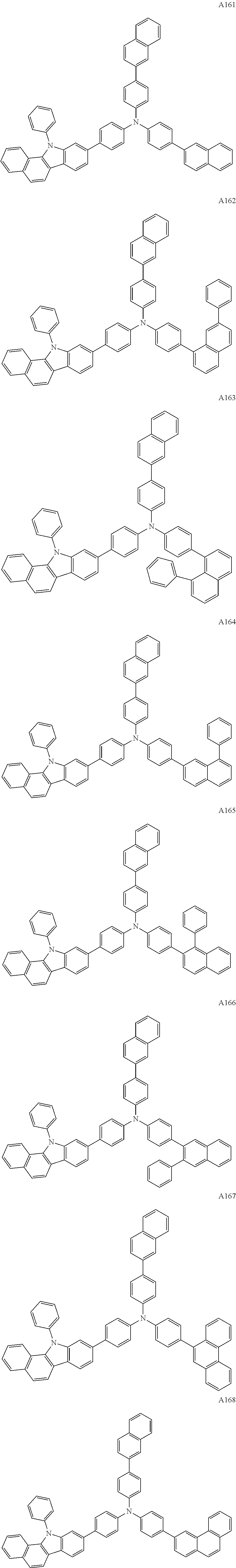

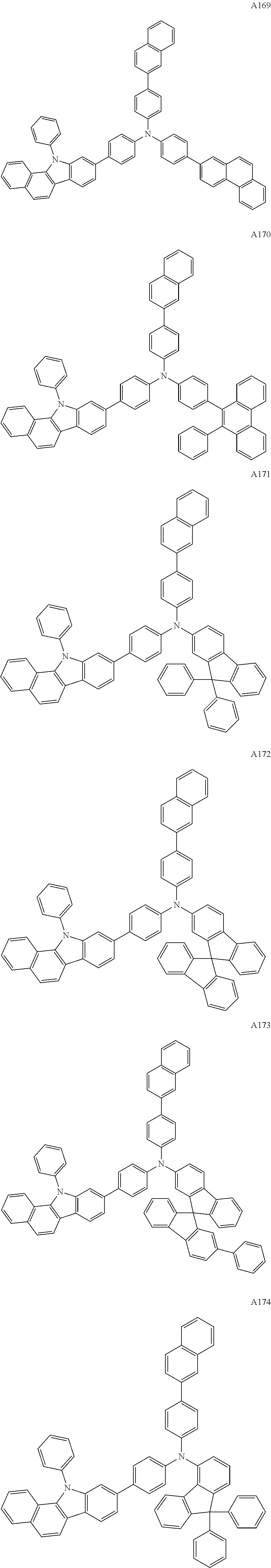

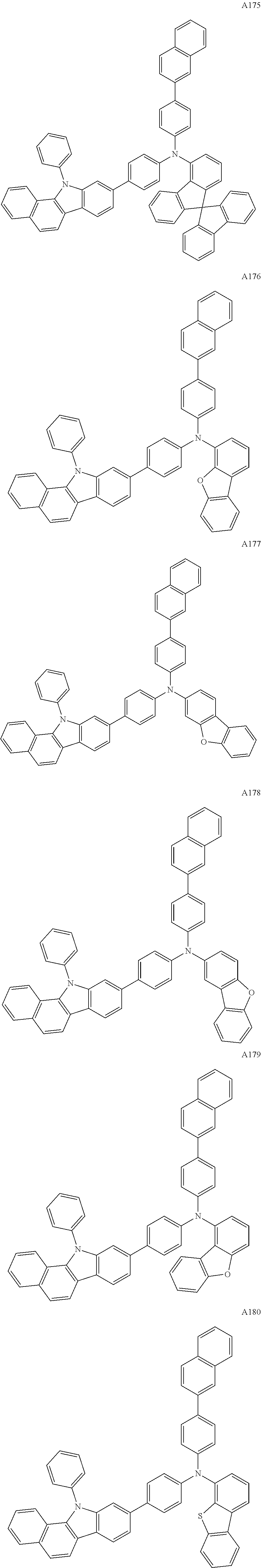

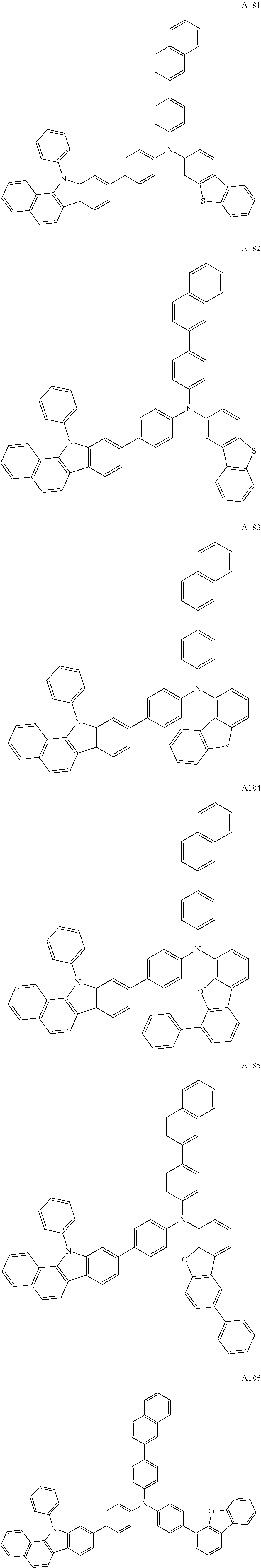

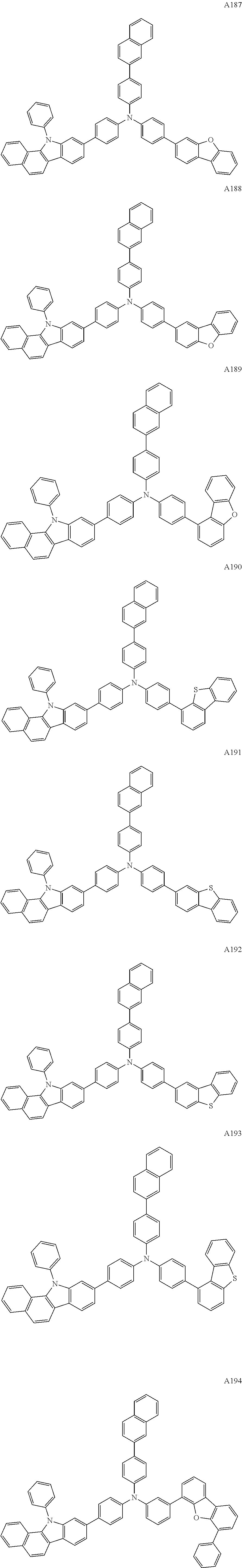

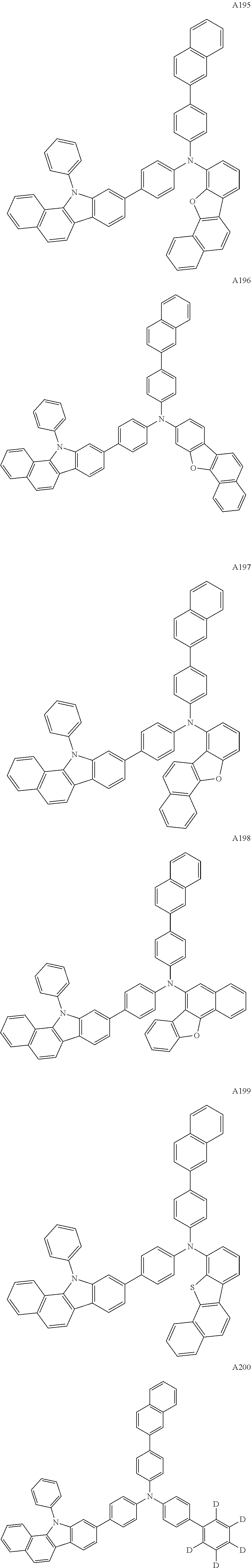

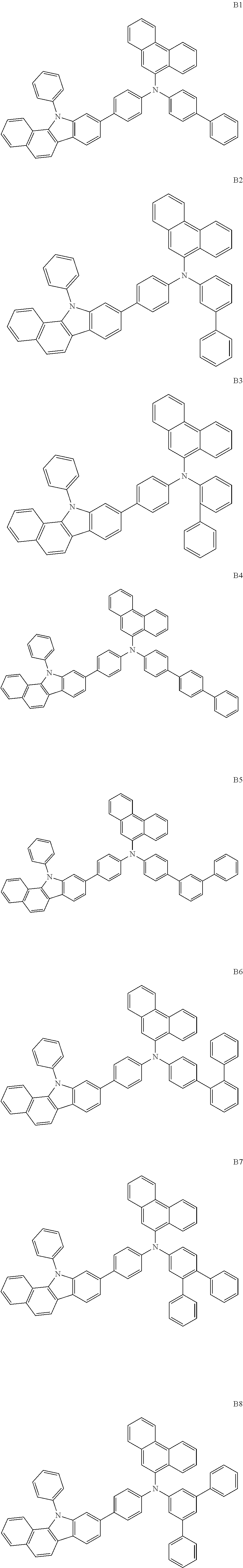

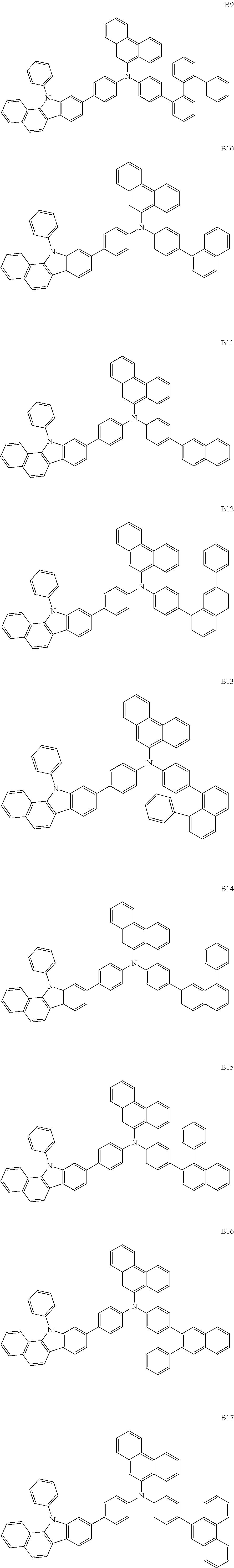

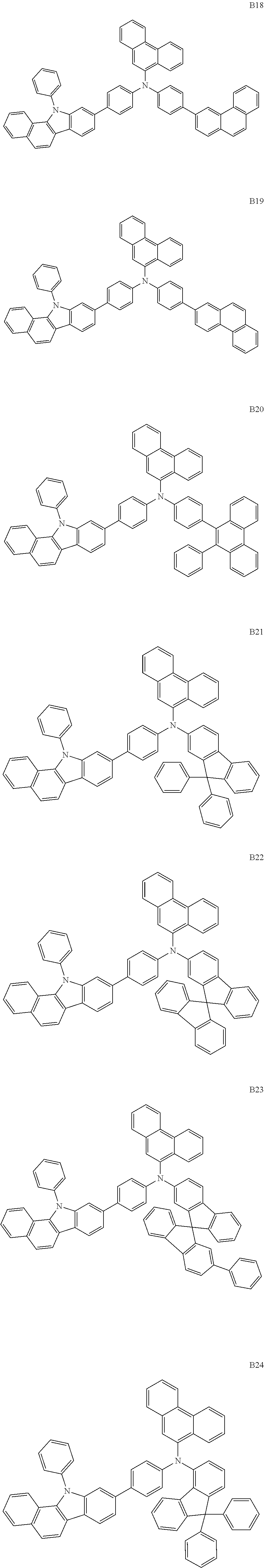

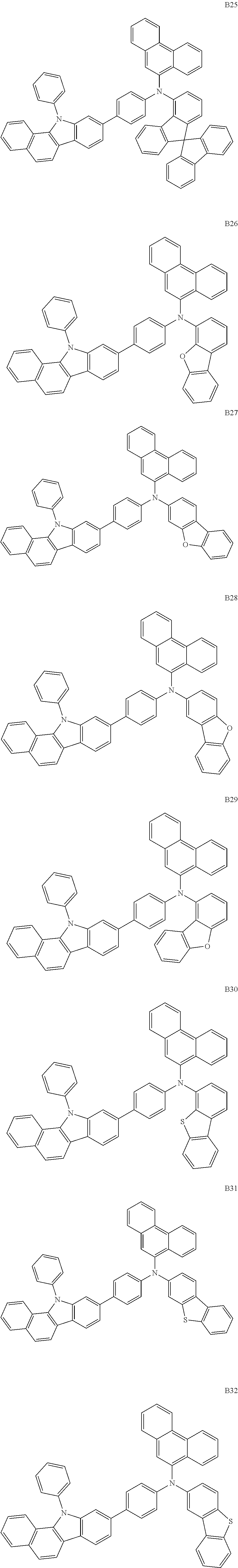

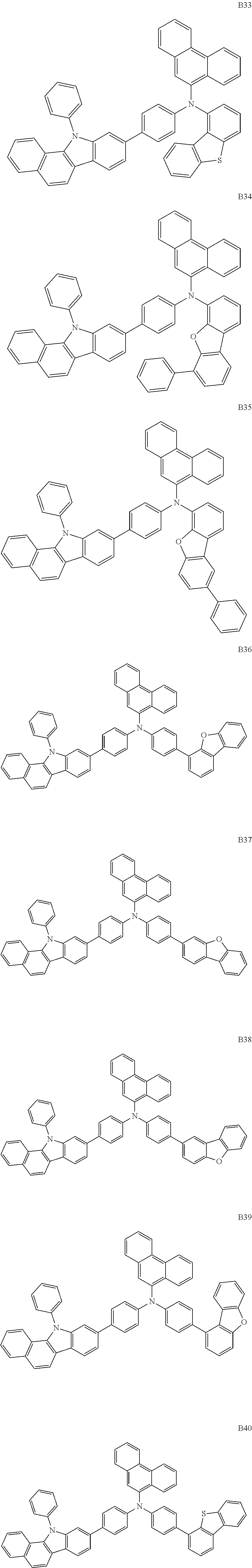

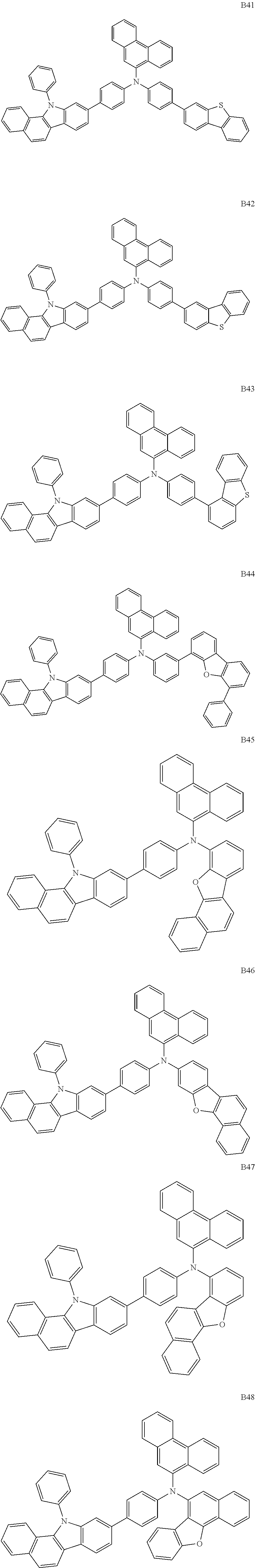

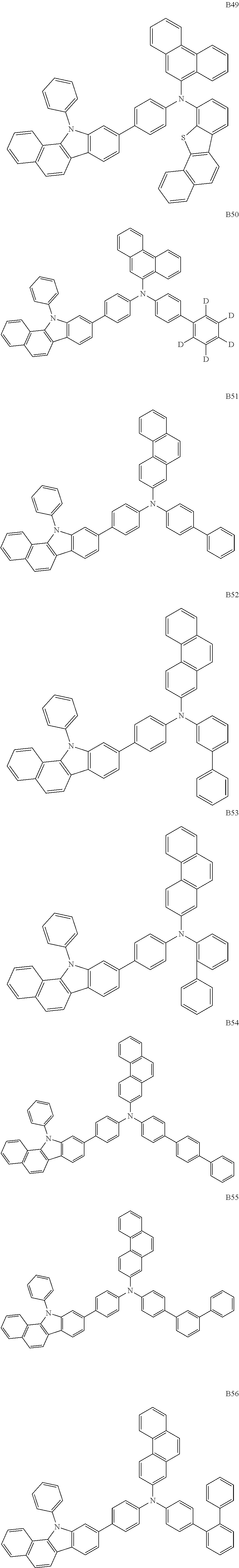

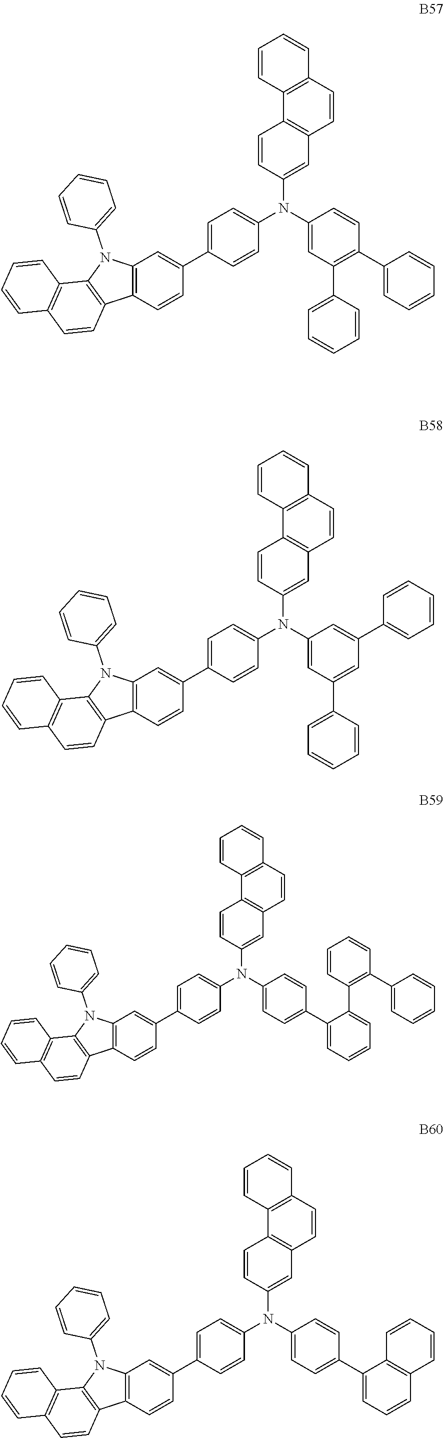 [SMILES: c1ccc(-c2ccc(N(c3ccc(-c4ccc5c6ccc7ccccc7c6n(-c6ccccc6)c5c4)cc3)c3ccc4c(ccc5ccccc54)c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(ccc5ccccc54)c3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(ccc5ccccc54)c3)cc2)cc1 ; c1ccc(-n2c3cc(-c4ccc(N(c5ccc(-c6cccc7ccccc67)cc5)c5ccc6c(ccc7ccccc76)c5)cc4)ccc3c3ccc4ccccc4c32)cc1]

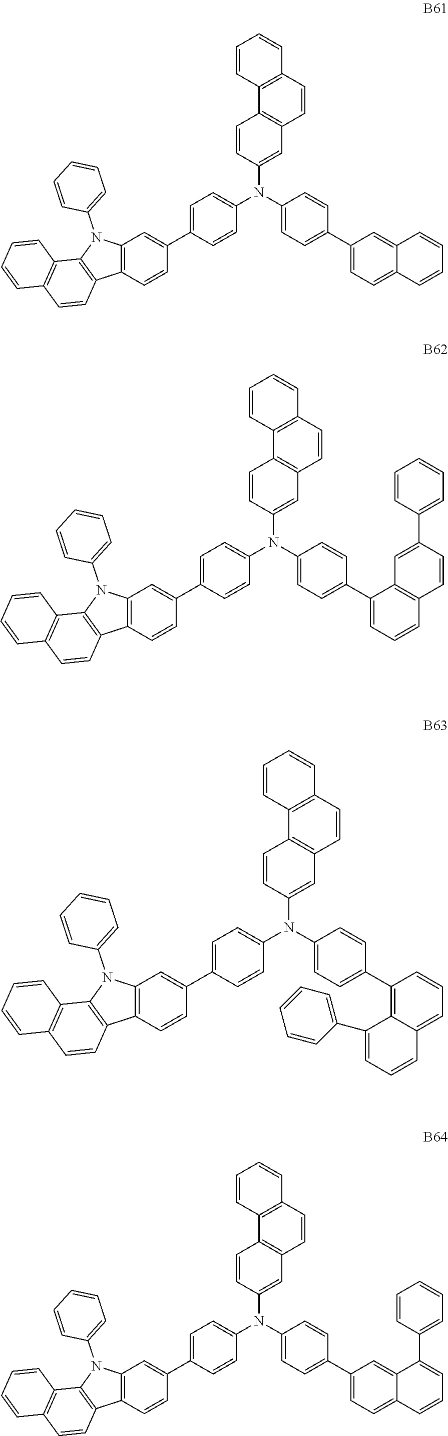 [SMILES: c1ccc(-n2c3cc(-c4ccc(N(c5ccc(-c6ccc7ccccc7c6)cc5)c5ccc6c(ccc7ccccc76)c5)cc4)ccc3c3ccc4ccccc4c32)cc1 ; c1ccc(-c2ccc3cccc(-c4ccc(N(c5ccc(-c6ccc7c8ccc9ccccc9c8n(-c8ccccc8)c7c6)cc5)c5ccc6c(ccc7ccccc76)c5)cc4)c3c2)cc1 ; c1ccc(-c2cccc3cccc(-c4ccc(N(c5ccc(-c6ccc7c8ccc9ccccc9c8n(-c8ccccc8)c7c6)cc5)c5ccc6c(ccc7ccccc76)c5)cc4)c23)cc1 ; c1ccc(-c2cccc3ccc(-c4ccc(N(c5ccc(-c6ccc7c8ccc9ccccc9c8n(-c8ccccc8)c7c6)cc5)c5ccc6c(ccc7ccccc76)c5)cc4)cc23)cc1]

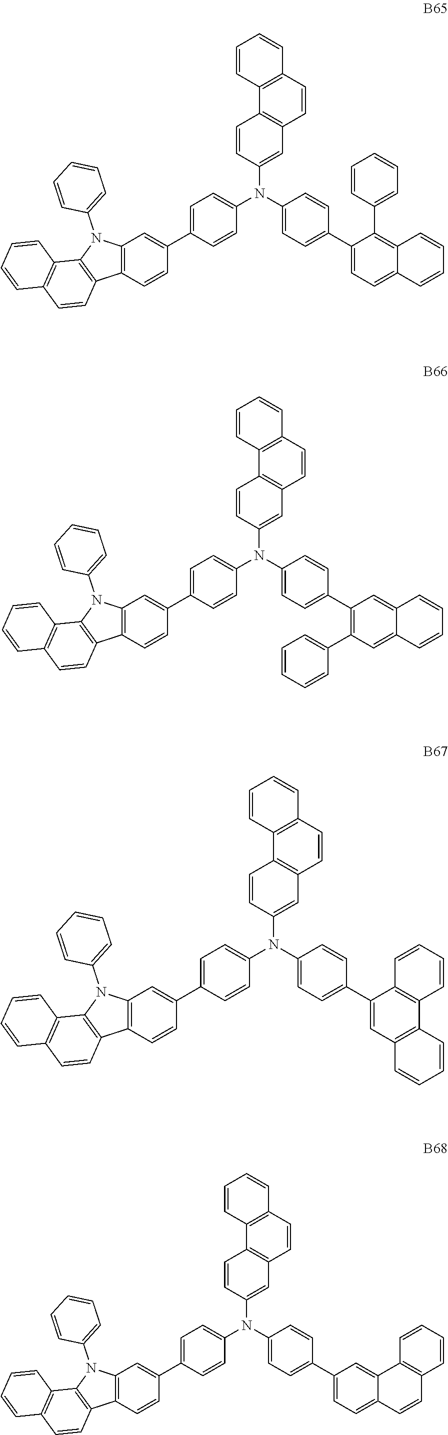 [SMILES: c1ccc(-c2c(-c3ccc(N(c4ccc(-c5ccc6c7ccc8ccccc8c7n(-c7ccccc7)c6c5)cc4)c4ccc5c(ccc6ccccc65)c4)cc3)ccc3ccccc23)cc1 ; c1ccc(-c2cc3ccccc3cc2-c2ccc(N(c3ccc(-c4ccc5c6ccc7ccccc7c6n(-c6ccccc6)c5c4)cc3)c3ccc4c(ccc5ccccc54)c3)cc2)cc1 ; c1ccc(-n2c3cc(-c4ccc(N(c5ccc(-c6cc7ccccc7c7ccccc67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7ccc8ccccc8c7c6)cc5)c5ccc6c(ccc7ccccc76)c5)cc4)ccc3c3ccc4ccccc4c32)cc1]

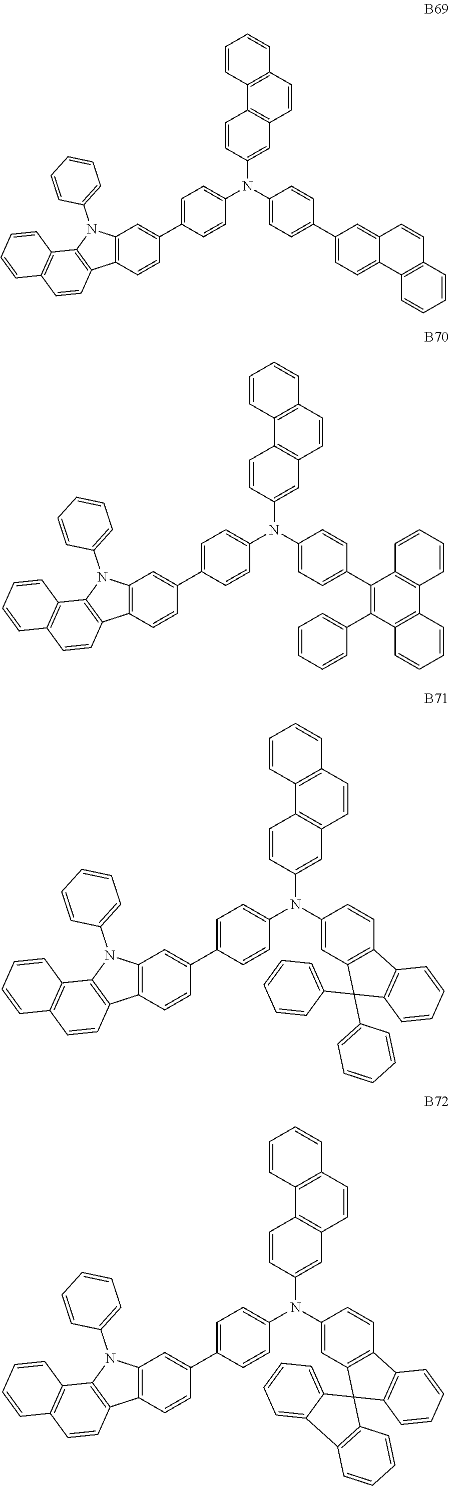 [SMILES: c1ccc(-n2c3cc(-c4ccc(N(c5ccc(-c6ccc7c(ccc8ccccc87)c6)cc5)c5ccc6c(ccc7ccccc76)c5)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4ccc5c(ccc6ccccc65)c4)cc3)c3ccccc3c3ccccc23)cc1 ; c1ccc(-n2c3cc(-c4ccc(N(c5ccc6c(c5)C(c5ccccc5)(c5ccccc5)c5ccccc5-6)c5ccc6c(ccc7ccccc76)c5)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6c(ccc7ccccc76)c5)cc4)ccc3c3ccc4ccccc4c32)cc1]

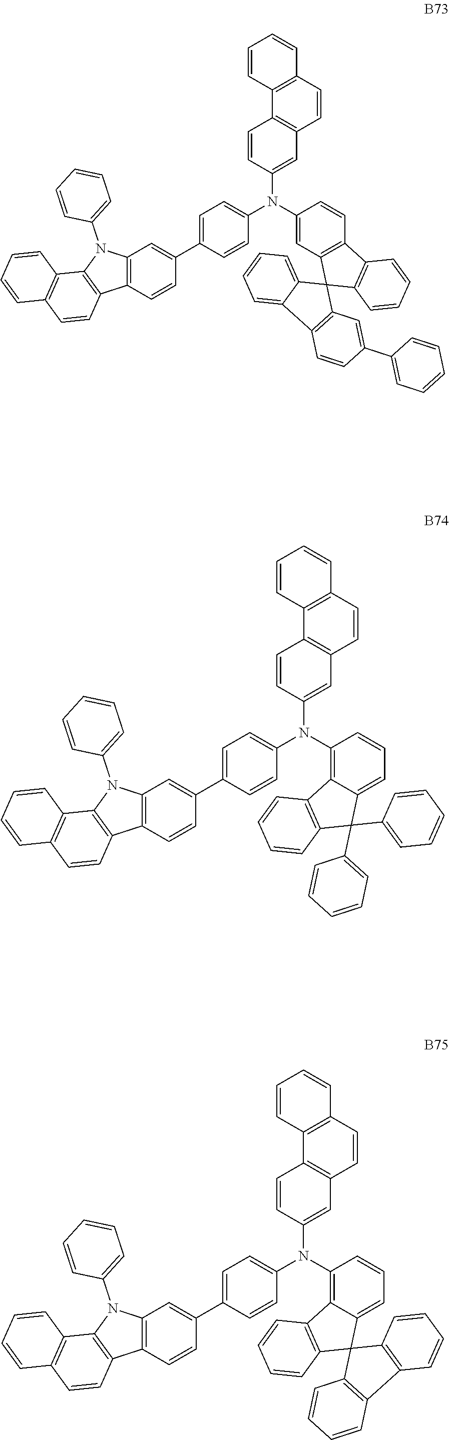 [SMILES: c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4ccc5c(ccc6ccccc65)c4)cc32)cc1 ; c1ccc(-n2c3cc(-c4ccc(N(c5ccc6c(ccc7ccccc76)c5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc6c(ccc7ccccc76)c5)c5cccc6c5-c5ccccc5C65c6ccccc6-c6ccccc65)cc4)ccc3c3ccc4ccccc4c32)cc1]

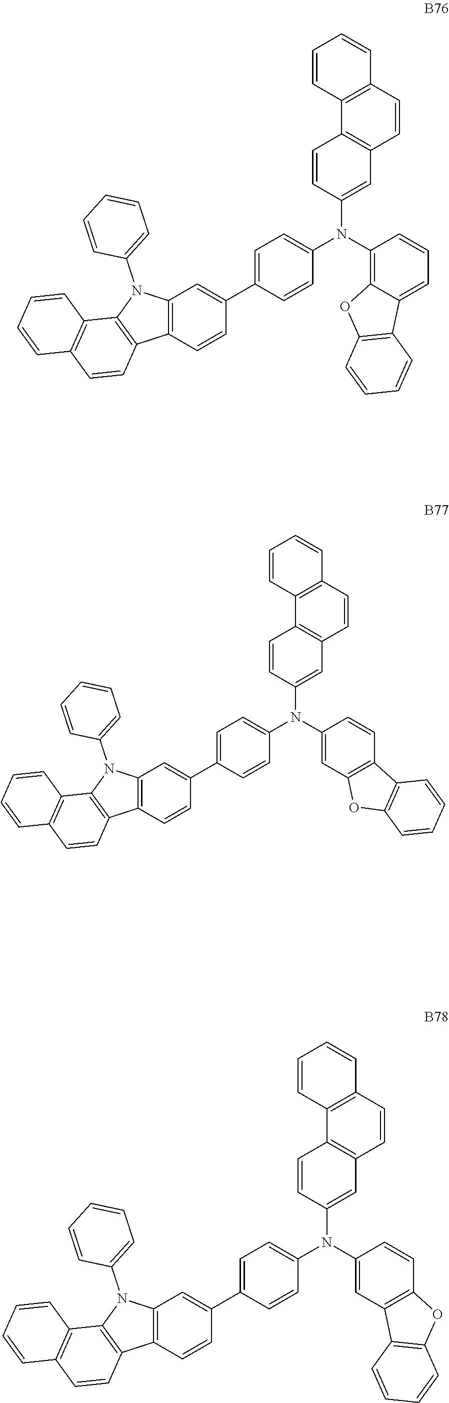 [SMILES: c1ccc(-n2c3cc(-c4ccc(N(c5ccc6c(ccc7ccccc76)c5)c5cccc6c5oc5ccccc56)cc4)ccc3c3ccc4ccccc4c32)cc1 ; c1ccc(-n2c3cc(-c4ccc(N(c5ccc6c(ccc7ccccc76)c5)c5ccc6c(c5)oc5ccccc56)cc4)ccc3c3ccc4ccccc4c32)cc1 ; c1ccc(-n2c3cc(-c4ccc(N(c5ccc6c(ccc7ccccc76)c5)c5ccc6oc7ccccc7c6c5)cc4)ccc3c3ccc4ccccc4c32)cc1]

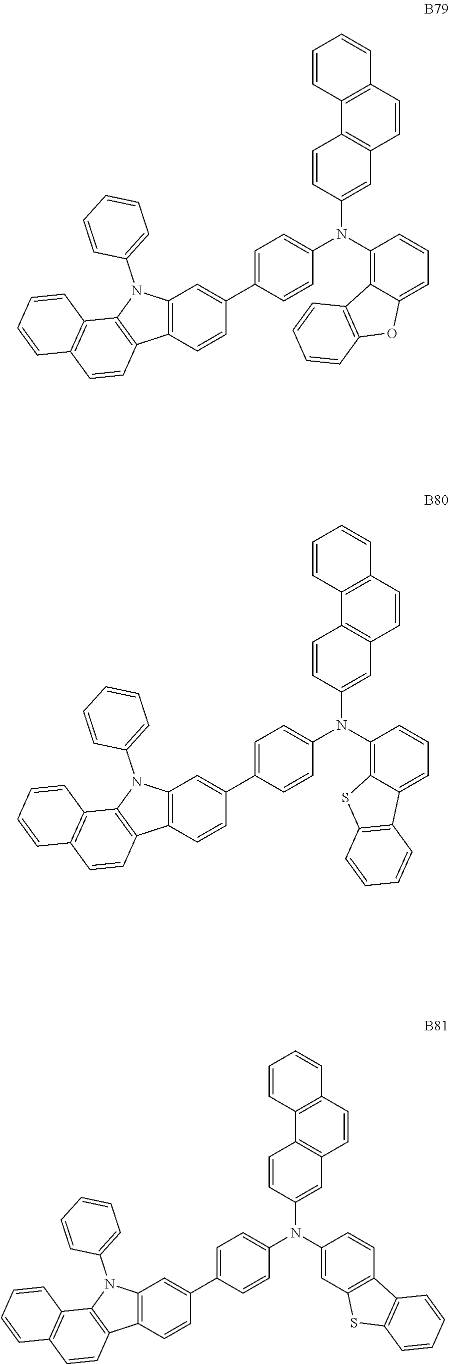 [SMILES: c1ccc(-n2c3cc(-c4ccc(N(c5ccc6c(ccc7ccccc76)c5)c5cccc6oc7ccccc7c56)cc4)ccc3c3ccc4ccccc4c32)cc1 ; c1ccc(-n2c3cc(-c4ccc(N(c5ccc6c(ccc7ccccc76)c5)c5cccc6c5sc5ccccc56)cc4)ccc3c3ccc4ccccc4c32)cc1 ; c1ccc(-n2c3cc(-c4ccc(N(c5ccc6c(ccc7ccccc76)c5)c5ccc6c(c5)sc5ccccc56)cc4)ccc3c3ccc4ccccc4c32)cc1]

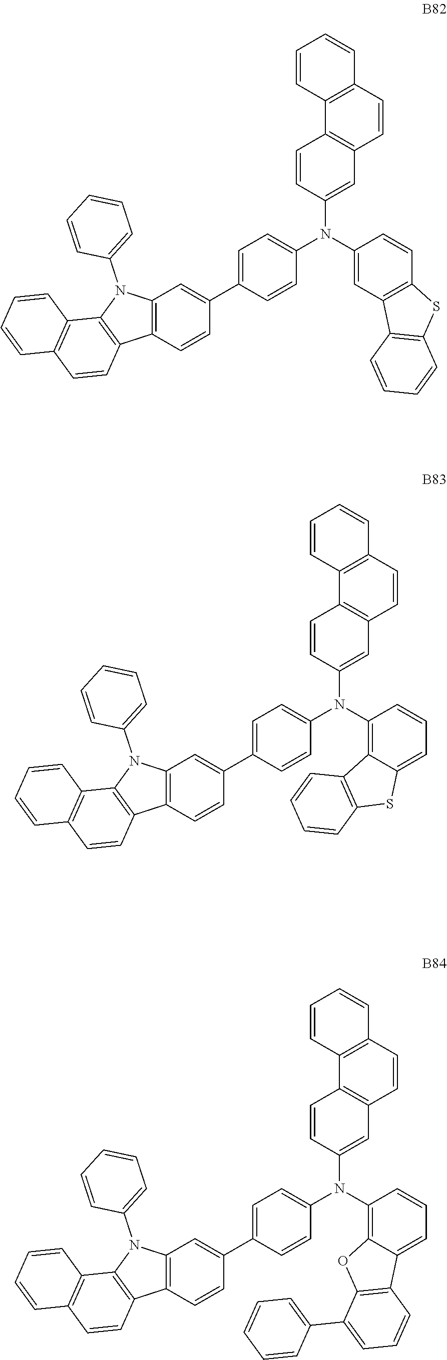 [SMILES: c1ccc(-n2c3cc(-c4ccc(N(c5ccc6c(ccc7ccccc76)c5)c5ccc6sc7ccccc7c6c5)cc4)ccc3c3ccc4ccccc4c32)cc1 ; c1ccc(-n2c3cc(-c4ccc(N(c5ccc6c(ccc7ccccc76)c5)c5cccc6sc7ccccc7c56)cc4)ccc3c3ccc4ccccc4c32)cc1 ; c1ccc(-c2cccc3c2oc2c(N(c4ccc(-c5ccc6c7ccc8ccccc8c7n(-c7ccccc7)c6c5)cc4)c4ccc5c(ccc6ccccc65)c4)cccc23)cc1]

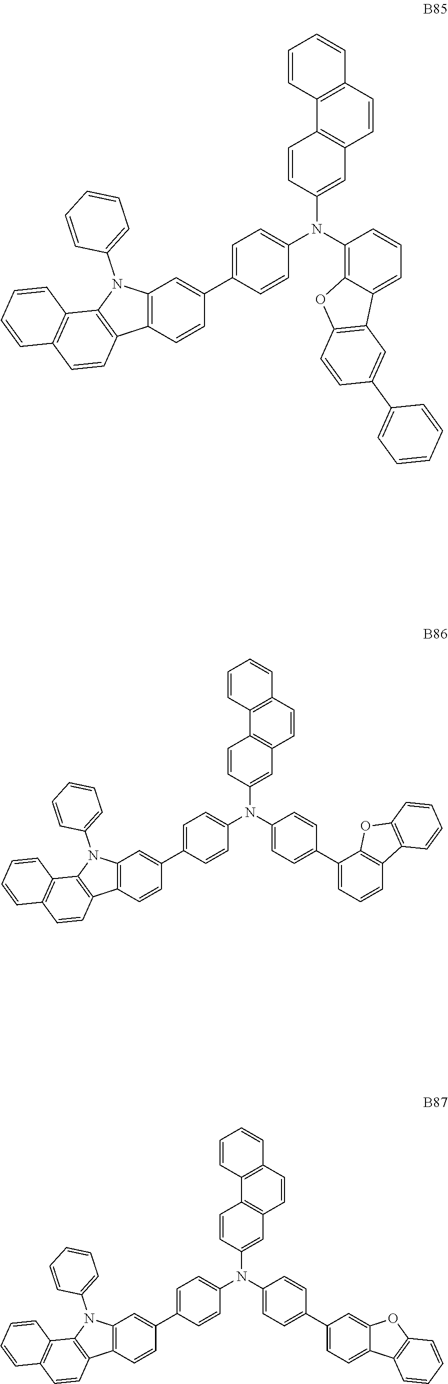 [SMILES: c1ccc(-c2ccc3oc4c(N(c5ccc(-c6ccc7c8ccc9ccccc9c8n(-c8ccccc8)c7c6)cc5)c5ccc6c(ccc7ccccc76)c5)cccc4c3c2)cc1 ; c1ccc(-n2c3cc(-c4ccc(N(c5ccc(-c6cccc7c6oc6ccccc67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7c(c6)oc6ccccc67)cc5)c5ccc6c(ccc7ccccc76)c5)cc4)ccc3c3ccc4ccccc4c32)cc1]

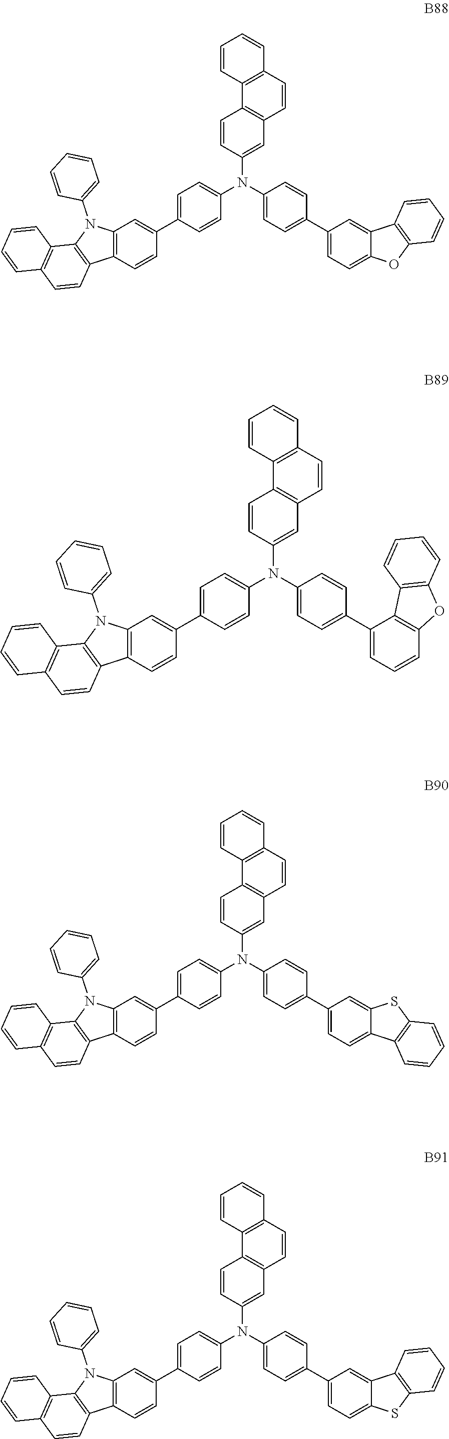 [SMILES: c1ccc(-n2c3cc(-c4ccc(N(c5ccc(-c6ccc7oc8ccccc8c7c6)cc5)c5ccc6c(ccc7ccccc76)c5)cc4)ccc3c3ccc4ccccc4c32)cc1 ; c1ccc(-n2c3cc(-c4ccc(N(c5ccc(-c6cccc7oc8ccccc8c67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7c(c6)sc6ccccc67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7sc8ccccc8c7c6)cc5)c5ccc6c(ccc7ccccc76)c5)cc4)ccc3c3ccc4ccccc4c32)cc1]

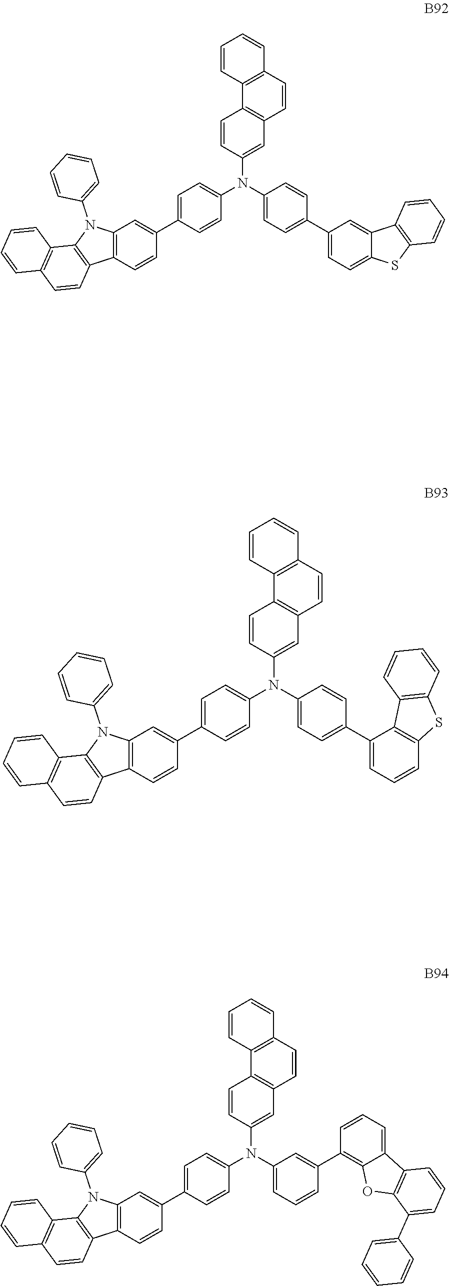 [SMILES: c1ccc(-n2c3cc(-c4ccc(N(c5ccc(-c6ccc7sc8ccccc8c7c6)cc5)c5ccc6c(ccc7ccccc76)c5)cc4)ccc3c3ccc4ccccc4c32)cc1 ; c1ccc(-n2c3cc(-c4ccc(N(c5ccc(-c6cccc7sc8ccccc8c67)cc5)c5ccc6c(ccc7ccccc76)c5)cc4)ccc3c3ccc4ccccc4c32)cc1 ; c1ccc(-c2cccc3c2oc2c(-c4cccc(N(c5ccc(-c6ccc7c8ccc9ccccc9c8n(-c8ccccc8)c7c6)cc5)c5ccc6c(ccc7ccccc76)c5)c4)cccc23)cc1]

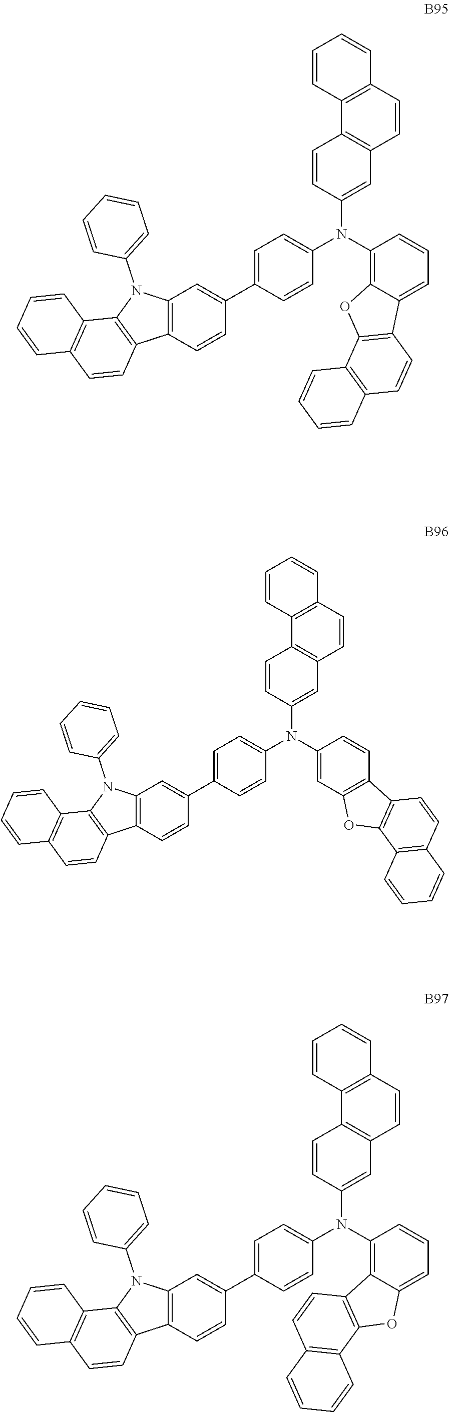 [SMILES: c1ccc(-n2c3cc(-c4ccc(N(c5ccc6c(ccc7ccccc76)c5)c5cccc6c5oc5c7ccccc7ccc65)cc4)ccc3c3ccc4ccccc4c32)cc1 ; c1ccc(-n2c3cc(-c4ccc(N(c5ccc6c(ccc7ccccc76)c5)c5ccc6c(c5)oc5c7ccccc7ccc65)cc4)ccc3c3ccc4ccccc4c32)cc1 ; c1ccc(-n2c3cc(-c4ccc(N(c5ccc6c(ccc7ccccc76)c5)c5cccc6oc7c8ccccc8ccc7c56)cc4)ccc3c3ccc4ccccc4c32)cc1]

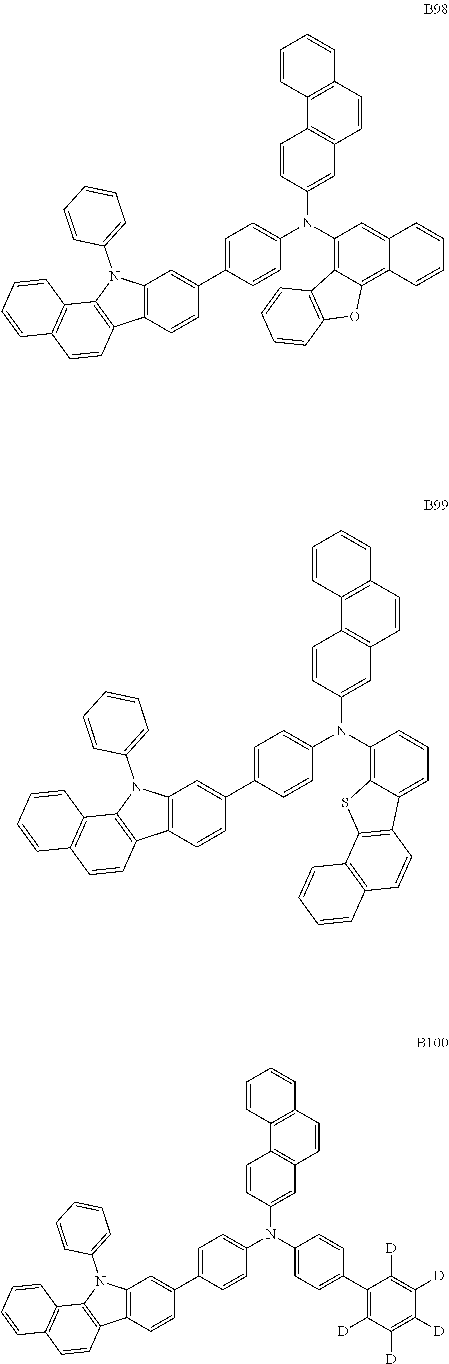 [SMILES: c1ccc(-n2c3cc(-c4ccc(N(c5ccc6c(ccc7ccccc76)c5)c5cc6ccccc6c6oc7ccccc7c56)cc4)ccc3c3ccc4ccccc4c32)cc1 ; c1ccc(-n2c3cc(-c4ccc(N(c5ccc6c(ccc7ccccc76)c5)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4c(ccc5ccccc54)c3)cc2)c([2H])c1[2H]]

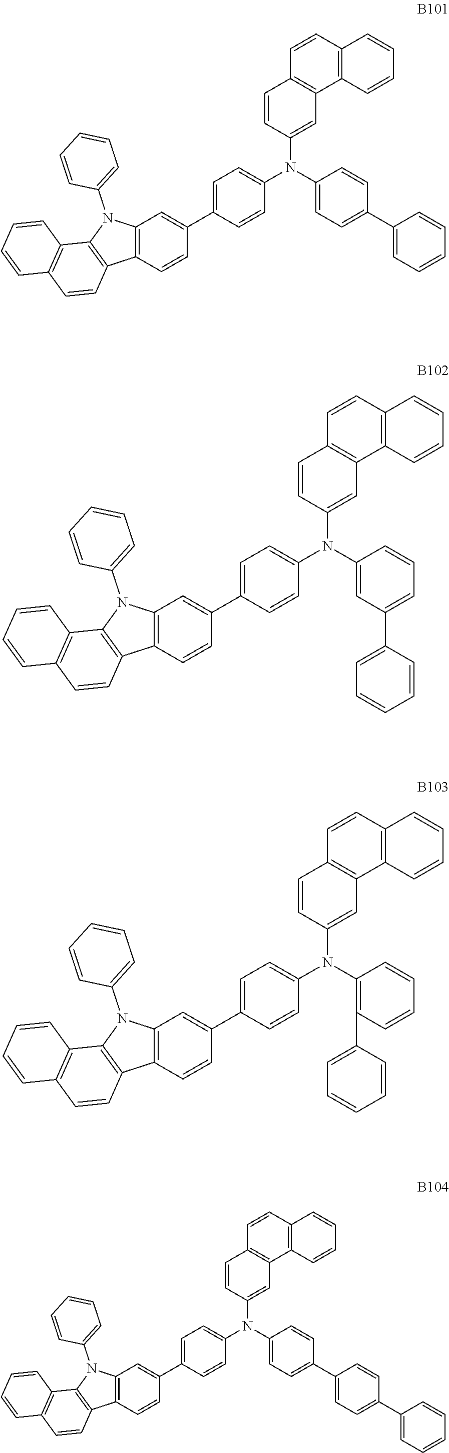 [SMILES: c1ccc(-c2ccc(N(c3ccc(-c4ccc5c6ccc7ccccc7c6n(-c6ccccc6)c5c4)cc3)c3ccc4ccc5ccccc5c4c3)cc2)cc1 ; c1ccc(-c2cccc(N(c3ccc(-c4ccc5c6ccc7ccccc7c6n(-c6ccccc6)c5c4)cc3)c3ccc4ccc5ccccc5c4c3)c2)cc1 ; c1ccc(-c2ccccc2N(c2ccc(-c3ccc4c5ccc6ccccc6c5n(-c5ccccc5)c4c3)cc2)c2ccc3ccc4ccccc4c3c2)cc1 ; c1ccc(-c2ccc(-c3ccc(N(c4ccc(-c5ccc6c7ccc8ccccc8c7n(-c7ccccc7)c6c5)cc4)c4ccc5ccc6ccccc6c5c4)cc3)cc2)cc1]

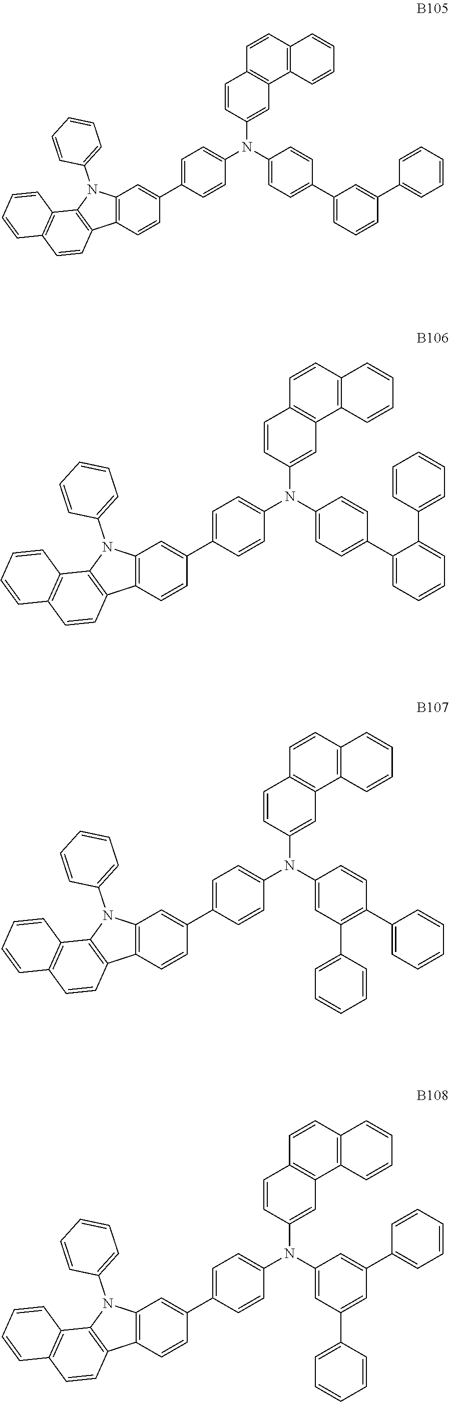 [SMILES: c1ccc(-c2cccc(-c3ccc(N(c4ccc(-c5ccc6c7ccc8ccccc8c7n(-c7ccccc7)c6c5)cc4)c4ccc5ccc6ccccc6c5c4)cc3)c2)cc1 ; c1ccc(-c2ccccc2-c2ccc(N(c3ccc(-c4ccc5c6ccc7ccccc7c6n(-c6ccccc6)c5c4)cc3)c3ccc4ccc5ccccc5c4c3)cc2)cc1 ; c1ccc(-c2ccc(N(c3ccc(-c4ccc5c6ccc7ccccc7c6n(-c6ccccc6)c5c4)cc3)c3ccc4ccc5ccccc5c4c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4ccc5ccccc5c4c3)c2)cc1]

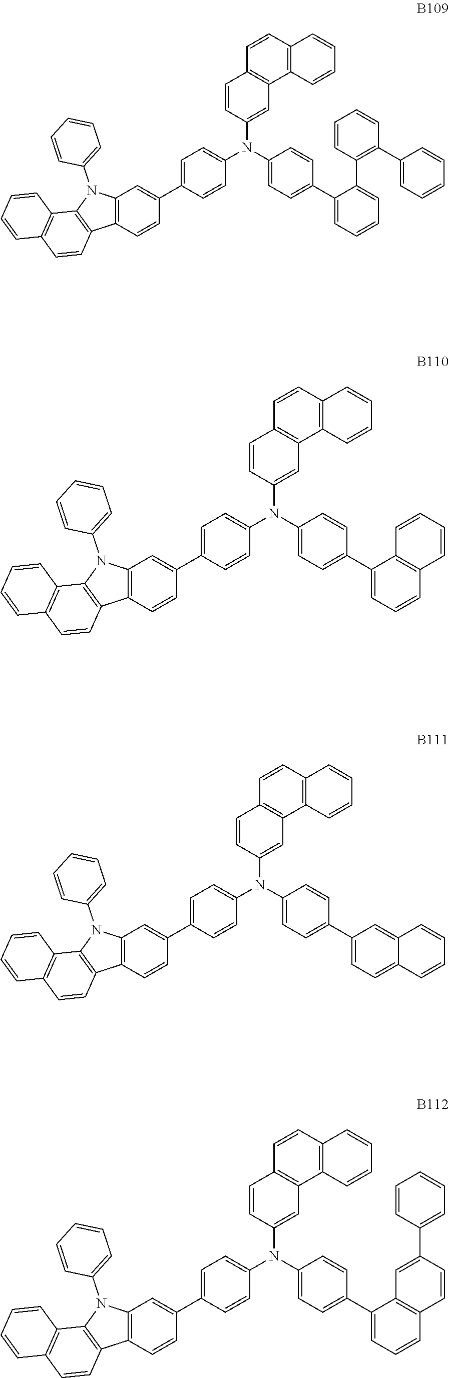 [SMILES: c1ccc(-c2ccccc2-c2ccccc2-c2ccc(N(c3ccc(-c4ccc5c6ccc7ccccc7c6n(-c6ccccc6)c5c4)cc3)c3ccc4ccc5ccccc5c4c3)cc2)cc1 ; c1ccc(-n2c3cc(-c4ccc(N(c5ccc(-c6cccc7ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7ccccc7c6)cc5)c5ccc6ccc7ccccc7c6c5)cc4)ccc3c3ccc4ccccc4c32)cc1 ; c1ccc(-c2ccc3cccc(-c4ccc(N(c5ccc(-c6ccc7c8ccc9ccccc9c8n(-c8ccccc8)c7c6)cc5)c5ccc6ccc7ccccc7c6c5)cc4)c3c2)cc1]

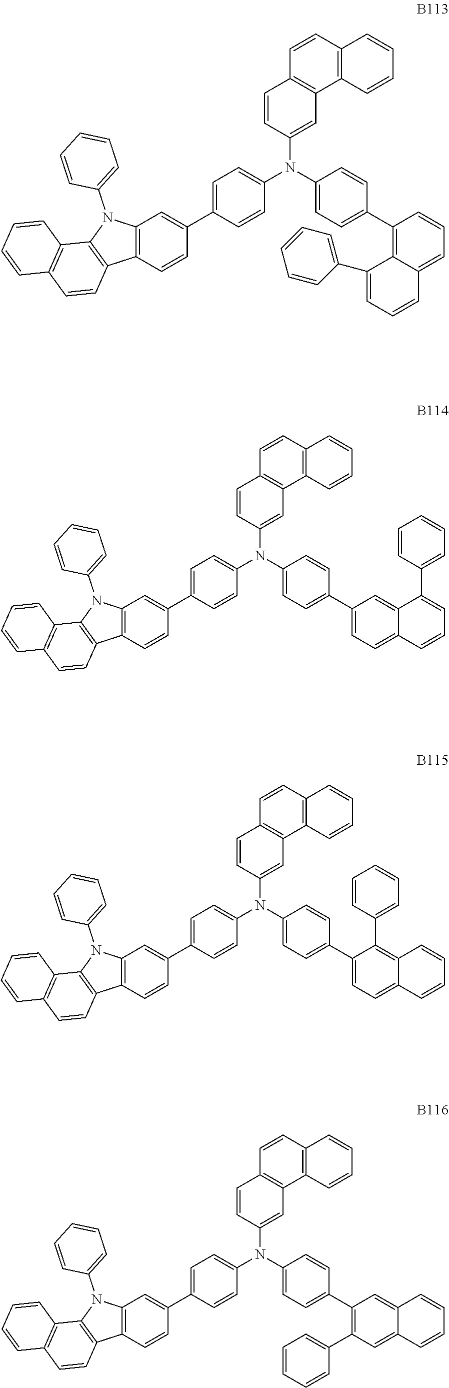 [SMILES: c1ccc(-c2cccc3cccc(-c4ccc(N(c5ccc(-c6ccc7c8ccc9ccccc9c8n(-c8ccccc8)c7c6)cc5)c5ccc6ccc7ccccc7c6c5)cc4)c23)cc1 ; c1ccc(-c2cccc3ccc(-c4ccc(N(c5ccc(-c6ccc7c8ccc9ccccc9c8n(-c8ccccc8)c7c6)cc5)c5ccc6ccc7ccccc7c6c5)cc4)cc23)cc1 ; c1ccc(-c2c(-c3ccc(N(c4ccc(-c5ccc6c7ccc8ccccc8c7n(-c7ccccc7)c6c5)cc4)c4ccc5ccc6ccccc6c5c4)cc3)ccc3ccccc23)cc1 ; c1ccc(-c2cc3ccccc3cc2-c2ccc(N(c3ccc(-c4ccc5c6ccc7ccccc7c6n(-c6ccccc6)c5c4)cc3)c3ccc4ccc5ccccc5c4c3)cc2)cc1]

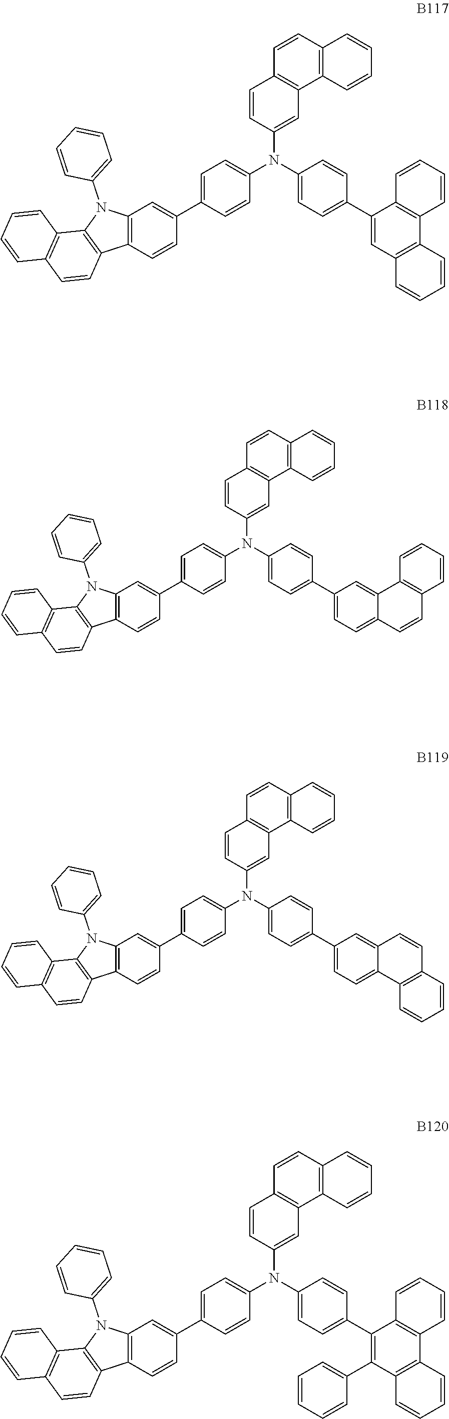 [SMILES: c1ccc(-n2c3cc(-c4ccc(N(c5ccc(-c6cc7ccccc7c7ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7ccc8ccccc8c7c6)cc5)c5ccc6ccc7ccccc7c6c5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6ccc7ccccc7c6c5)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4ccc5ccc6ccccc6c5c4)cc3)c3ccccc3c3ccccc23)cc1]

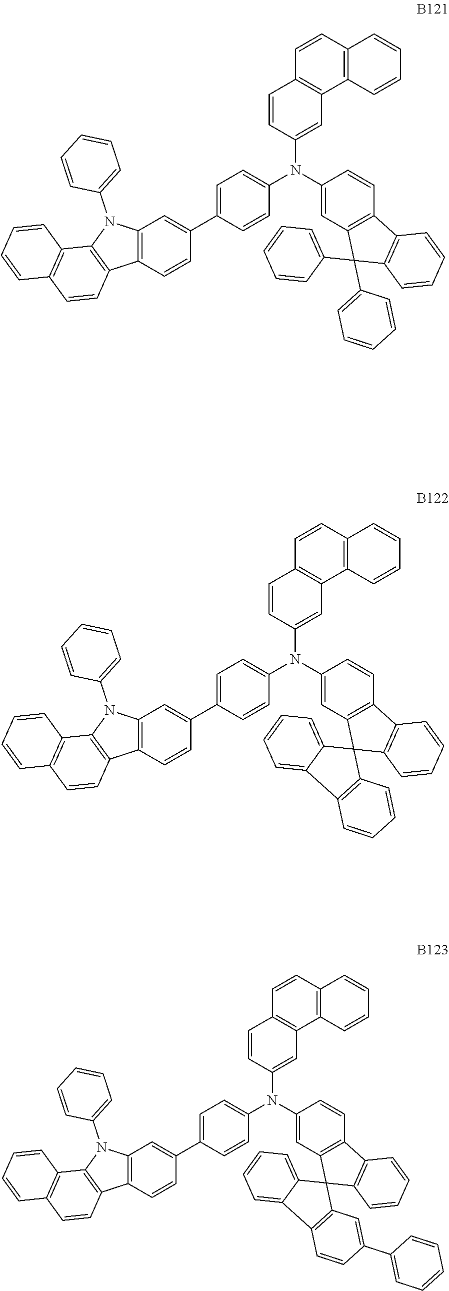 [SMILES: c1ccc(-n2c3cc(-c4ccc(N(c5ccc6c(c5)C(c5ccccc5)(c5ccccc5)c5ccccc5-6)c5ccc6ccc7ccccc7c6c5)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6ccc7ccccc7c6c5)cc4)ccc3c3ccc4ccccc4c32)cc1 ; c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4ccc5ccc6ccccc6c5c4)cc32)cc1]

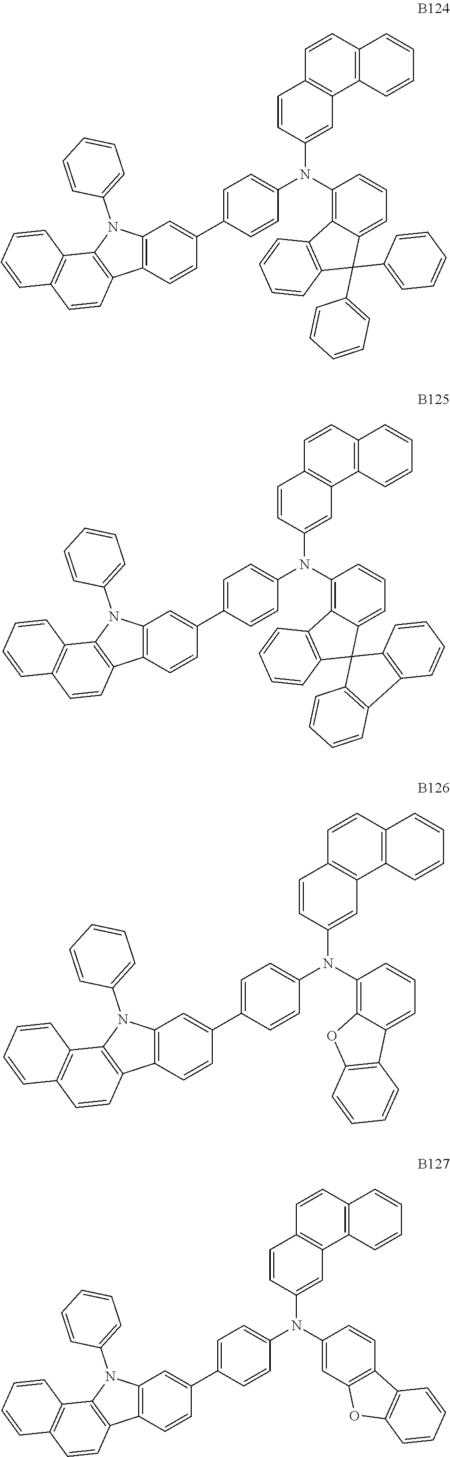 [SMILES: c1ccc(-n2c3cc(-c4ccc(N(c5ccc6ccc7ccccc7c6c5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc6ccc7ccccc7c6c5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc6ccc7ccccc7c6c5)c5cccc6c5oc5ccccc56)cc4)ccc3c3ccc4ccccc4c32)cc1 ; c1ccc(-n2c3cc(-c4ccc(N(c5ccc6c(c5)oc5ccccc56)c5ccc6ccc7ccccc7c6c5)cc4)ccc3c3ccc4ccccc4c32)cc1]

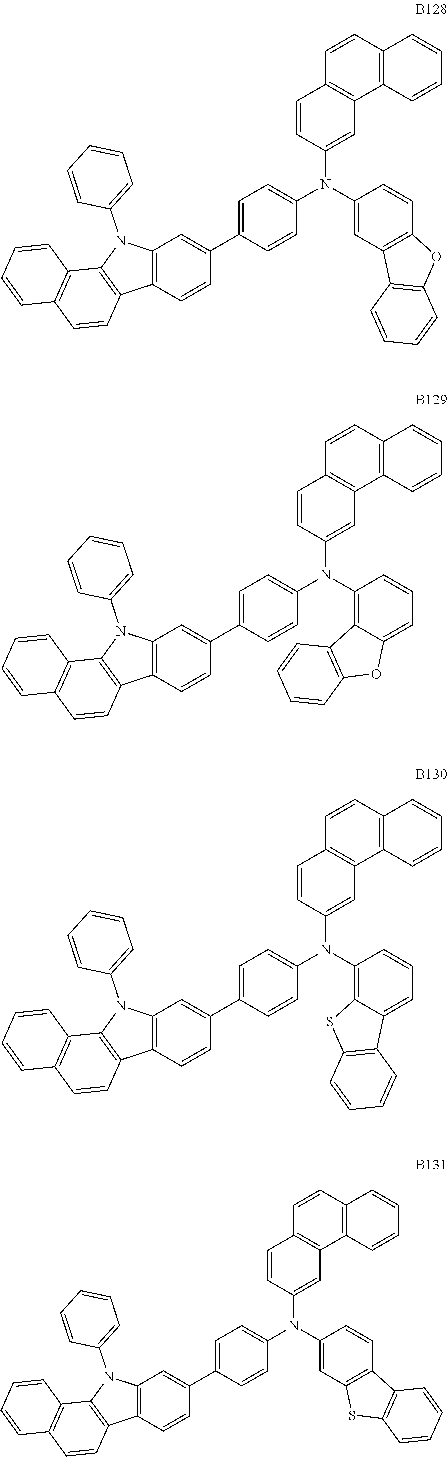 [SMILES: c1ccc(-n2c3cc(-c4ccc(N(c5ccc6ccc7ccccc7c6c5)c5ccc6oc7ccccc7c6c5)cc4)ccc3c3ccc4ccccc4c32)cc1 ; c1ccc(-n2c3cc(-c4ccc(N(c5ccc6ccc7ccccc7c6c5)c5cccc6oc7ccccc7c56)cc4)ccc3c3ccc4ccccc4c32)cc1 ; c1ccc(-n2c3cc(-c4ccc(N(c5ccc6ccc7ccccc7c6c5)c5cccc6c5sc5ccccc56)cc4)ccc3c3ccc4ccccc4c32)cc1 ; c1ccc(-n2c3cc(-c4ccc(N(c5ccc6c(c5)sc5ccccc56)c5ccc6ccc7ccccc7c6c5)cc4)ccc3c3ccc4ccccc4c32)cc1]

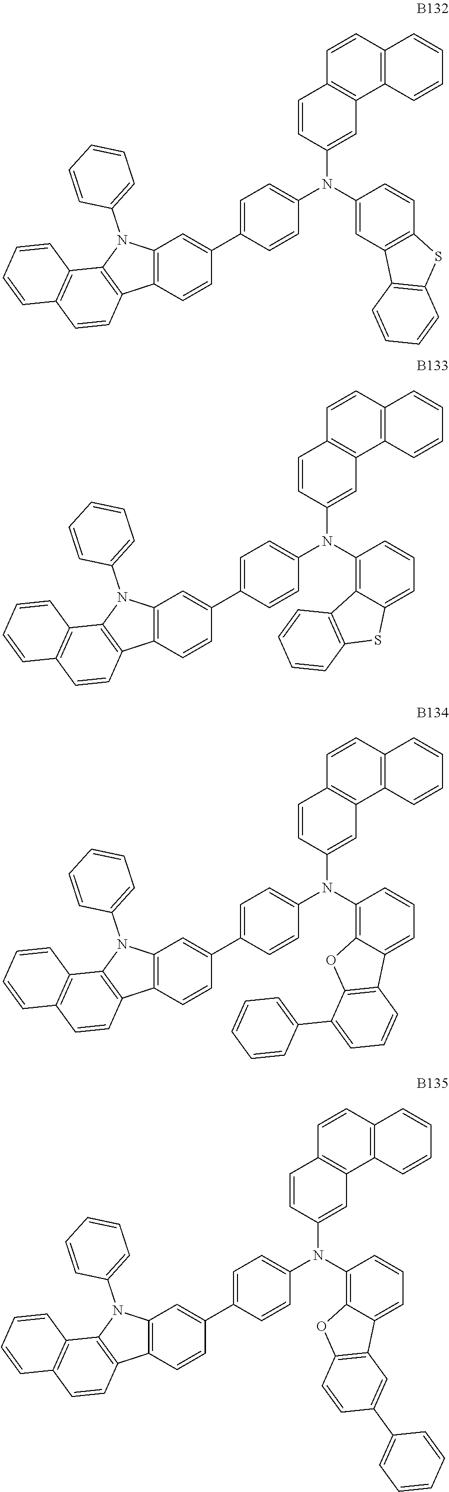 [SMILES: c1ccc(-n2c3cc(-c4ccc(N(c5ccc6ccc7ccccc7c6c5)c5ccc6sc7ccccc7c6c5)cc4)ccc3c3ccc4ccccc4c32)cc1 ; c1ccc(-n2c3cc(-c4ccc(N(c5ccc6ccc7ccccc7c6c5)c5cccc6sc7ccccc7c56)cc4)ccc3c3ccc4ccccc4c32)cc1 ; c1ccc(-c2cccc3c2oc2c(N(c4ccc(-c5ccc6c7ccc8ccccc8c7n(-c7ccccc7)c6c5)cc4)c4ccc5ccc6ccccc6c5c4)cccc23)cc1 ; c1ccc(-c2ccc3oc4c(N(c5ccc(-c6ccc7c8ccc9ccccc9c8n(-c8ccccc8)c7c6)cc5)c5ccc6ccc7ccccc7c6c5)cccc4c3c2)cc1]

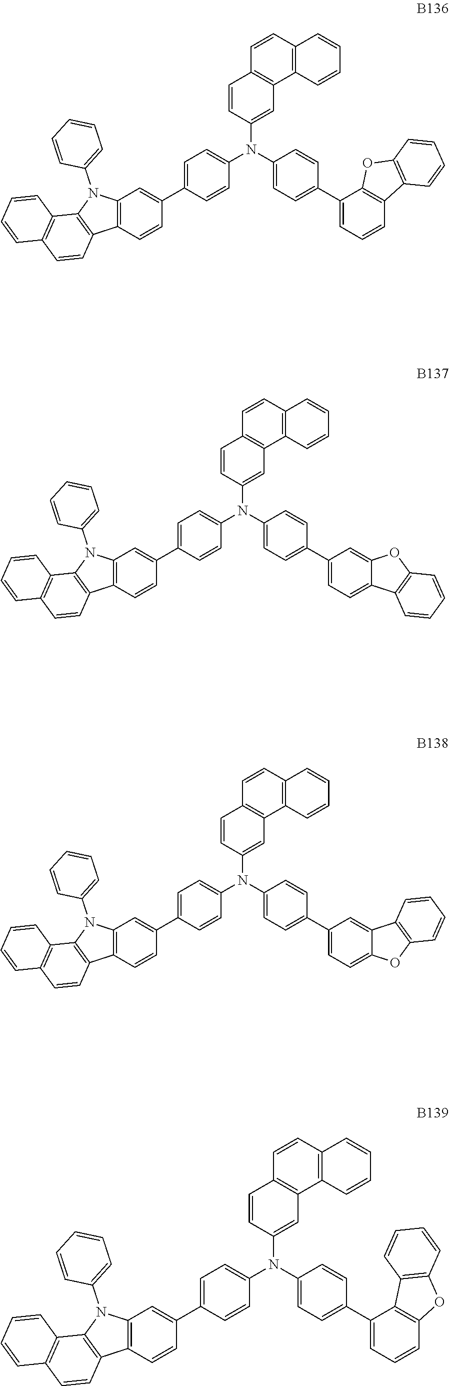 [SMILES: c1ccc(-n2c3cc(-c4ccc(N(c5ccc(-c6cccc7c6oc6ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7c(c6)oc6ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7oc8ccccc8c7c6)cc5)c5ccc6ccc7ccccc7c6c5)cc4)ccc3c3ccc4ccccc4c32)cc1 ; c1ccc(-n2c3cc(-c4ccc(N(c5ccc(-c6cccc7oc8ccccc8c67)cc5)c5ccc6ccc7ccccc7c6c5)cc4)ccc3c3ccc4ccccc4c32)cc1]

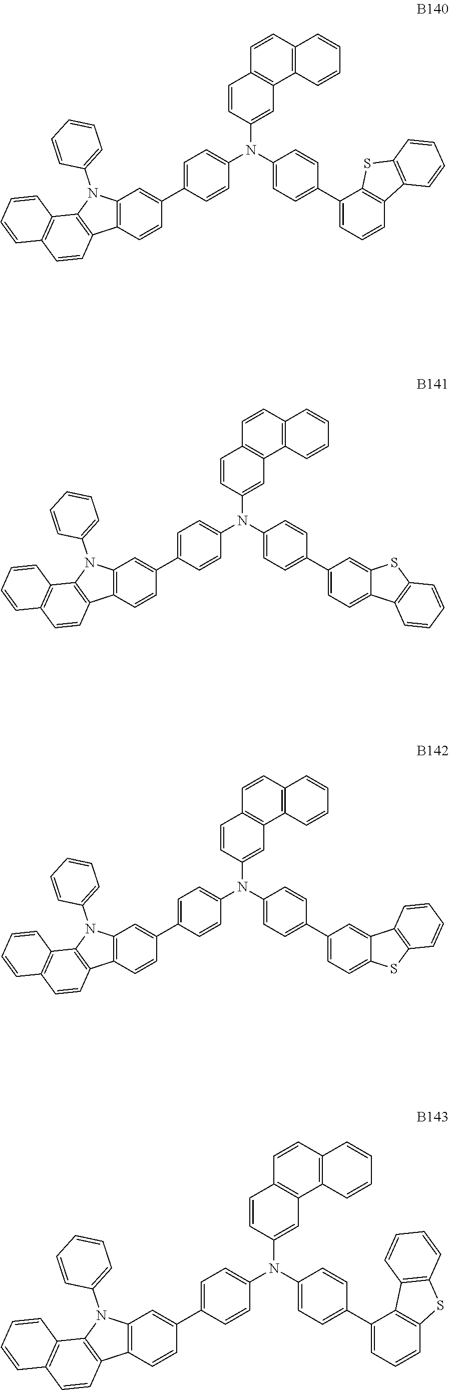 [SMILES: c1ccc(-n2c3cc(-c4ccc(N(c5ccc(-c6cccc7c6sc6ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7c(c6)sc6ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7sc8ccccc8c7c6)cc5)c5ccc6ccc7ccccc7c6c5)cc4)ccc3c3ccc4ccccc4c32)cc1 ; c1ccc(-n2c3cc(-c4ccc(N(c5ccc(-c6cccc7sc8ccccc8c67)cc5)c5ccc6ccc7ccccc7c6c5)cc4)ccc3c3ccc4ccccc4c32)cc1]

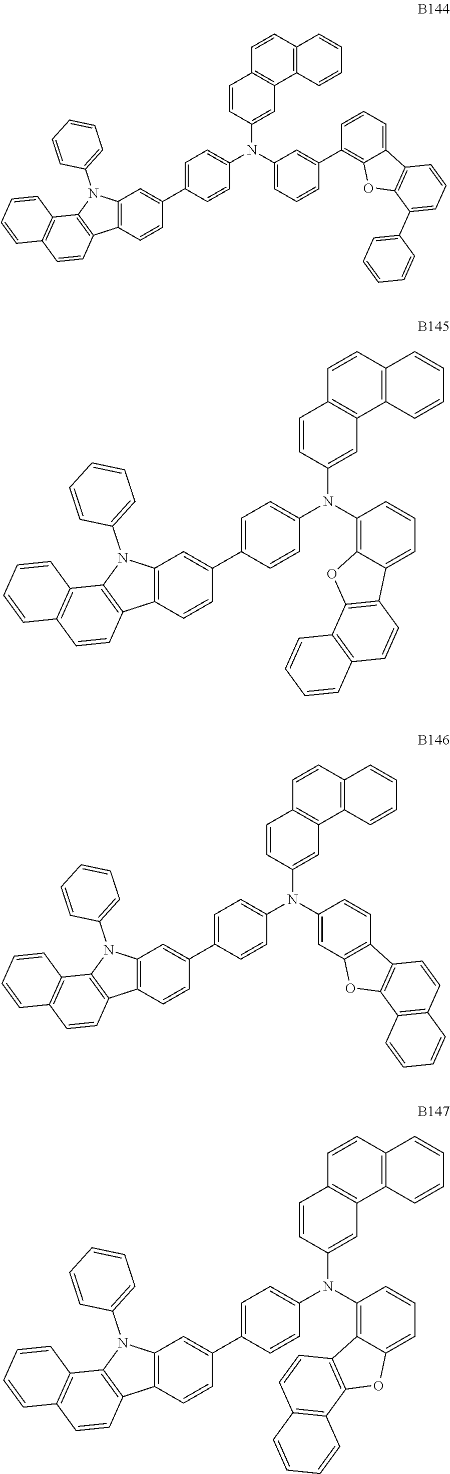 [SMILES: c1ccc(-c2cccc3c2oc2c(-c4cccc(N(c5ccc(-c6ccc7c8ccc9ccccc9c8n(-c8ccccc8)c7c6)cc5)c5ccc6ccc7ccccc7c6c5)c4)cccc23)cc1 ; c1ccc(-n2c3cc(-c4ccc(N(c5ccc6ccc7ccccc7c6c5)c5cccc6c5oc5c7ccccc7ccc65)cc4)ccc3c3ccc4ccccc4c32)cc1 ; c1ccc(-n2c3cc(-c4ccc(N(c5ccc6c(c5)oc5c7ccccc7ccc65)c5ccc6ccc7ccccc7c6c5)cc4)ccc3c3ccc4ccccc4c32)cc1 ; c1ccc(-n2c3cc(-c4ccc(N(c5ccc6ccc7ccccc7c6c5)c5cccc6oc7c8ccccc8ccc7c56)cc4)ccc3c3ccc4ccccc4c32)cc1]

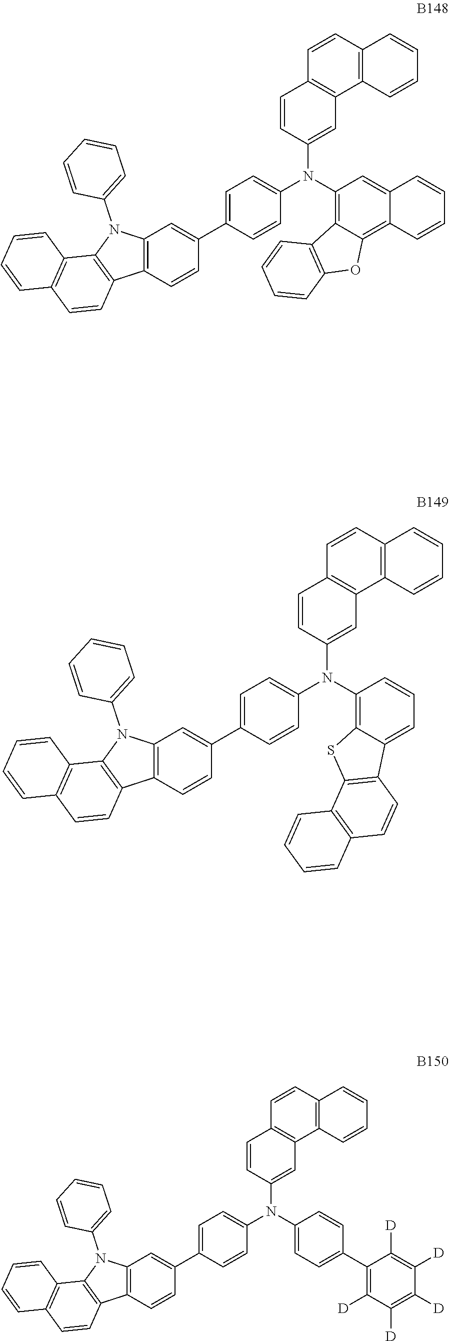 [SMILES: c1ccc(-n2c3cc(-c4ccc(N(c5ccc6ccc7ccccc7c6c5)c5cc6ccccc6c6oc7ccccc7c56)cc4)ccc3c3ccc4ccccc4c32)cc1 ; c1ccc(-n2c3cc(-c4ccc(N(c5ccc6ccc7ccccc7c6c5)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4ccc5ccccc5c4c3)cc2)c([2H])c1[2H]]

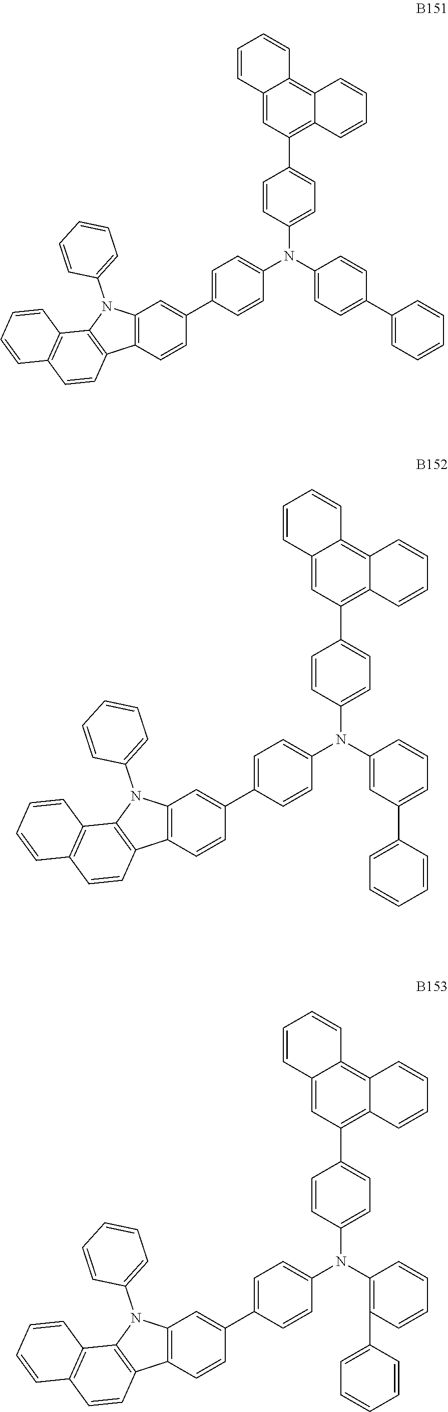 [SMILES: c1ccc(-c2ccc(N(c3ccc(-c4ccc5c6ccc7ccccc7c6n(-c6ccccc6)c5c4)cc3)c3ccc(-c4cc5ccccc5c5ccccc45)cc3)cc2)cc1 ; c1ccc(-c2cccc(N(c3ccc(-c4ccc5c6ccc7ccccc7c6n(-c6ccccc6)c5c4)cc3)c3ccc(-c4cc5ccccc5c5ccccc45)cc3)c2)cc1 ; c1ccc(-c2ccccc2N(c2ccc(-c3ccc4c5ccc6ccccc6c5n(-c5ccccc5)c4c3)cc2)c2ccc(-c3cc4ccccc4c4ccccc34)cc2)cc1]

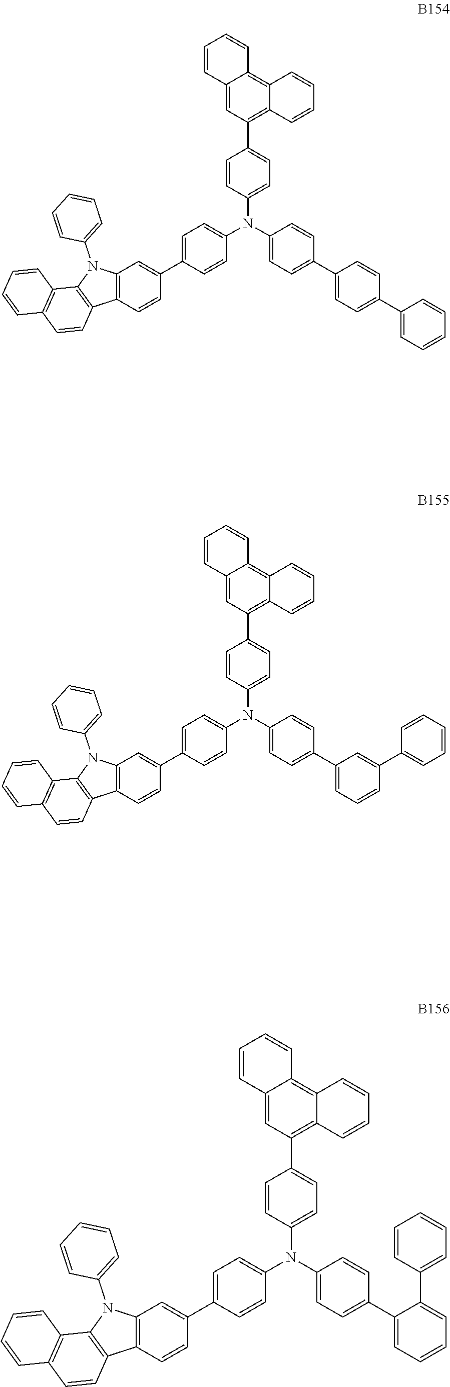 [SMILES: c1ccc(-c2ccc(-c3ccc(N(c4ccc(-c5ccc6c7ccc8ccccc8c7n(-c7ccccc7)c6c5)cc4)c4ccc(-c5cc6ccccc6c6ccccc56)cc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc(-c5cc6ccccc6c6ccccc56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cc5ccccc5c5ccccc45)cc3)cc2)cc1]

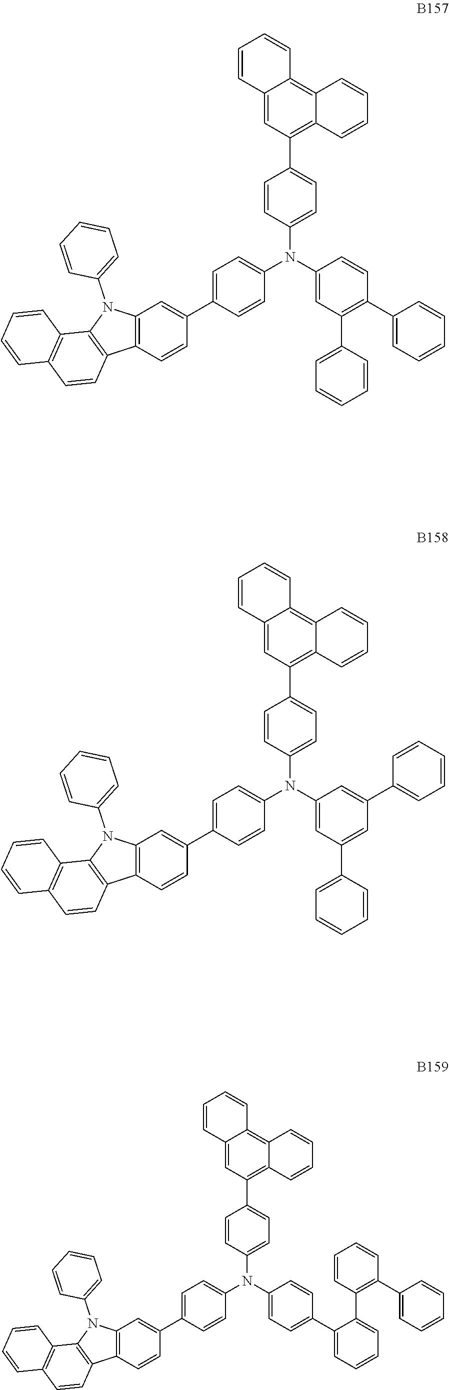 [SMILES: c1ccc(-c2ccc(N(c3ccc(-c4ccc5c6ccc7ccccc7c6n(-c6ccccc6)c5c4)cc3)c3ccc(-c4cc5ccccc5c5ccccc45)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cc5ccccc5c5ccccc45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cc5ccccc5c5ccccc45)cc3)cc2)cc1]

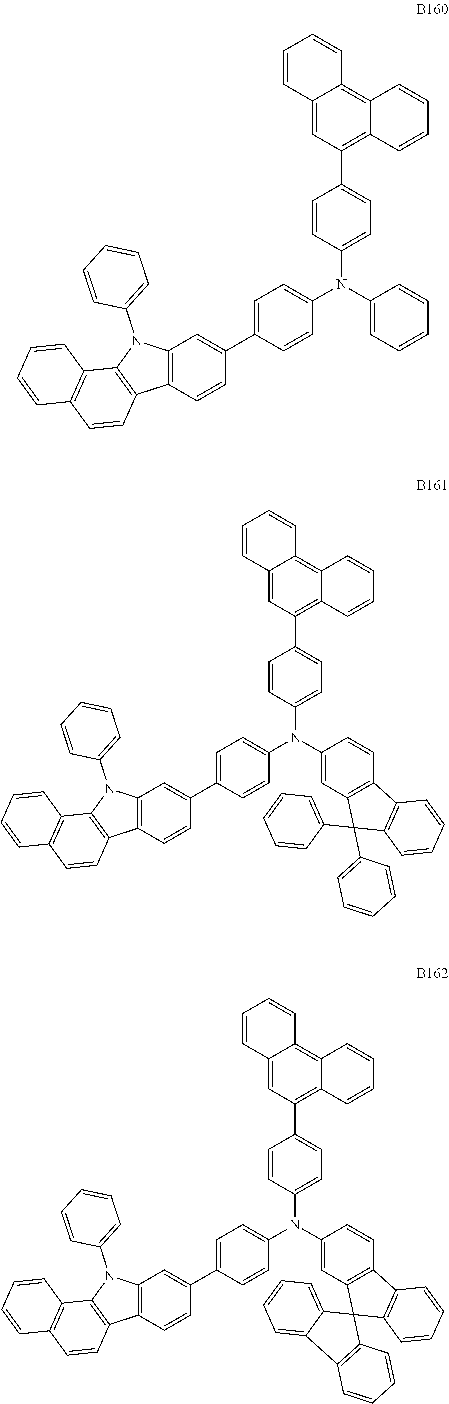 [SMILES: c1ccc(N(c2ccc(-c3ccc4c5ccc6ccccc6c5n(-c5ccccc5)c4c3)cc2)c2ccc(-c3cc4ccccc4c4ccccc34)cc2)cc1 ; c1ccc(-n2c3cc(-c4ccc(N(c5ccc(-c6cc7ccccc7c7ccccc67)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6cc7ccccc7c7ccccc67)cc5)c5ccc6c(c5)C5(c7ccccc7-c7ccccc75)c5ccccc5-6)cc4)ccc3c3ccc4ccccc4c32)cc1]

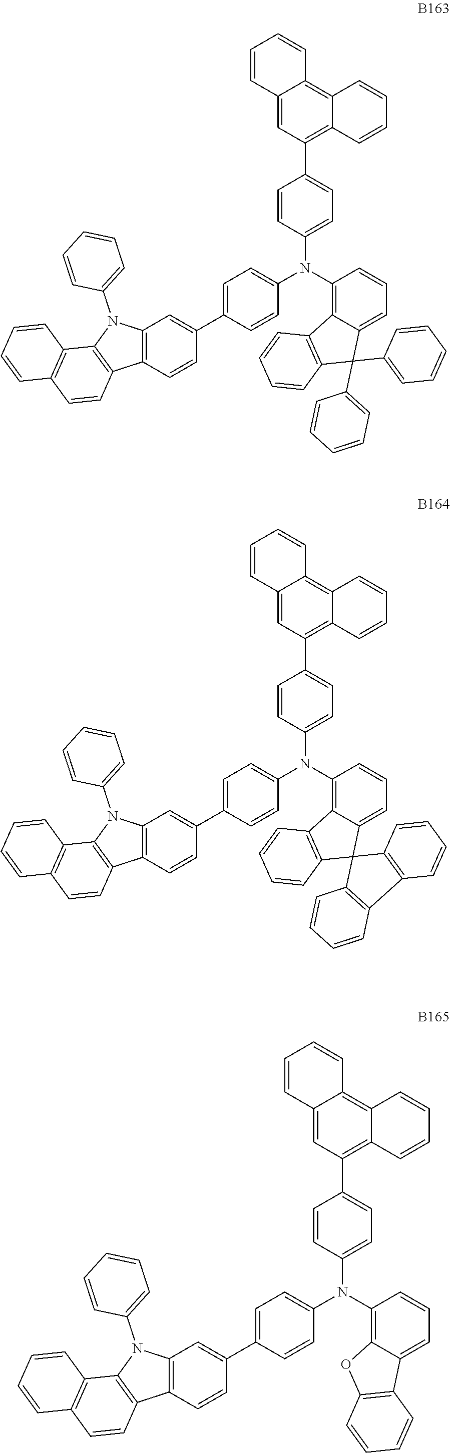 [SMILES: c1ccc(-n2c3cc(-c4ccc(N(c5ccc(-c6cc7ccccc7c7ccccc67)cc5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc(-c6cc7ccccc7c7ccccc67)cc5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc(-c6cc7ccccc7c7ccccc67)cc5)c5cccc6c5oc5ccccc56)cc4)ccc3c3ccc4ccccc4c32)cc1]

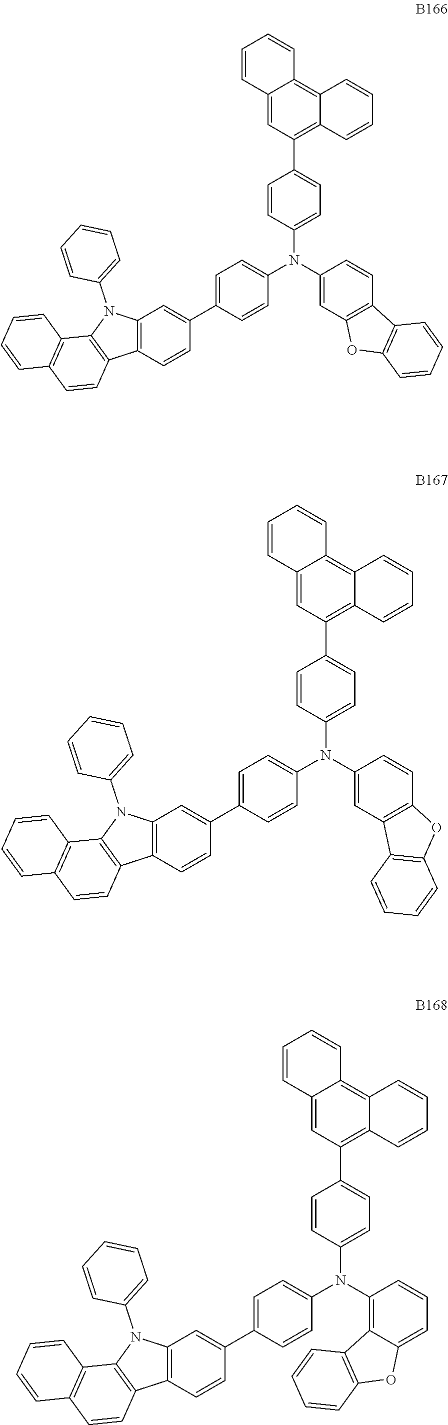 [SMILES: c1ccc(-n2c3cc(-c4ccc(N(c5ccc(-c6cc7ccccc7c7ccccc67)cc5)c5ccc6c(c5)oc5ccccc56)cc4)ccc3c3ccc4ccccc4c32)cc1 ; c1ccc(-n2c3cc(-c4ccc(N(c5ccc(-c6cc7ccccc7c7ccccc67)cc5)c5ccc6oc7ccccc7c6c5)cc4)ccc3c3ccc4ccccc4c32)cc1 ; c1ccc(-n2c3cc(-c4ccc(N(c5ccc(-c6cc7ccccc7c7ccccc67)cc5)c5cccc6oc7ccccc7c56)cc4)ccc3c3ccc4ccccc4c32)cc1]

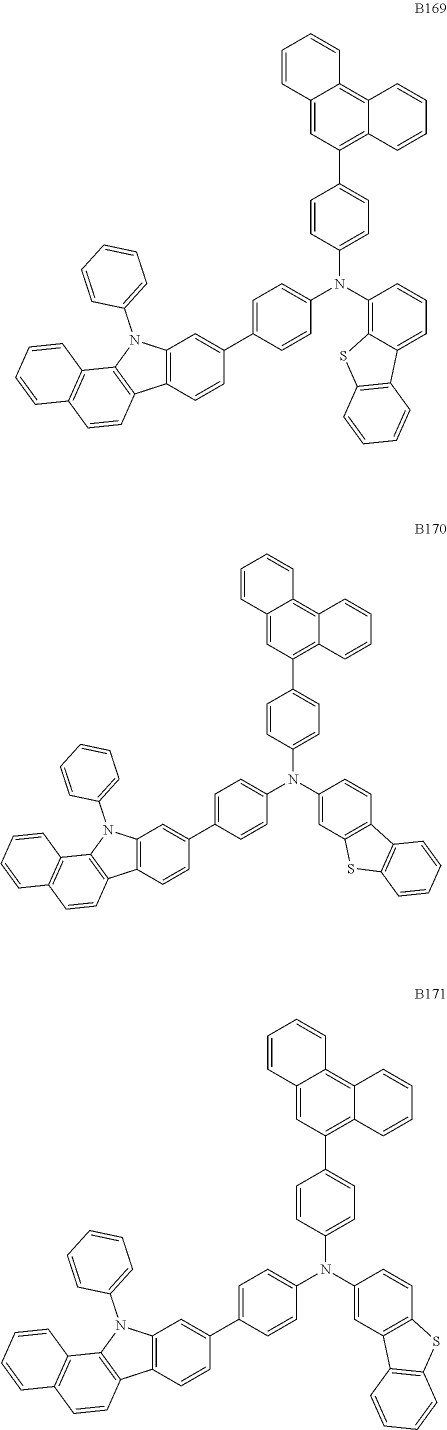 [SMILES: c1ccc(-n2c3cc(-c4ccc(N(c5ccc(-c6cc7ccccc7c7ccccc67)cc5)c5cccc6c5sc5ccccc56)cc4)ccc3c3ccc4ccccc4c32)cc1 ; c1ccc(-n2c3cc(-c4ccc(N(c5ccc(-c6cc7ccccc7c7ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6cc7ccccc7c7ccccc67)cc5)c5ccc6sc7ccccc7c6c5)cc4)ccc3c3ccc4ccccc4c32)cc1]

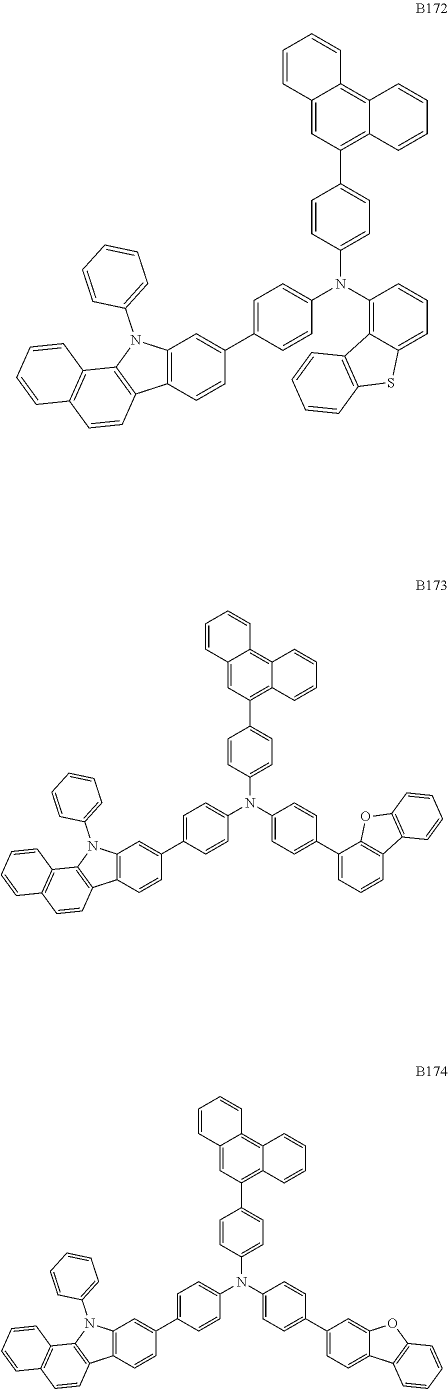 [SMILES: c1ccc(-n2c3cc(-c4ccc(N(c5ccc(-c6cc7ccccc7c7ccccc67)cc5)c5cccc6sc7ccccc7c56)cc4)ccc3c3ccc4ccccc4c32)cc1 ; c1ccc(-n2c3cc(-c4ccc(N(c5ccc(-c6cc7ccccc7c7ccccc67)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7c(c6)oc6ccccc67)cc5)c5ccc(-c6cc7ccccc7c7ccccc67)cc5)cc4)ccc3c3ccc4ccccc4c32)cc1]

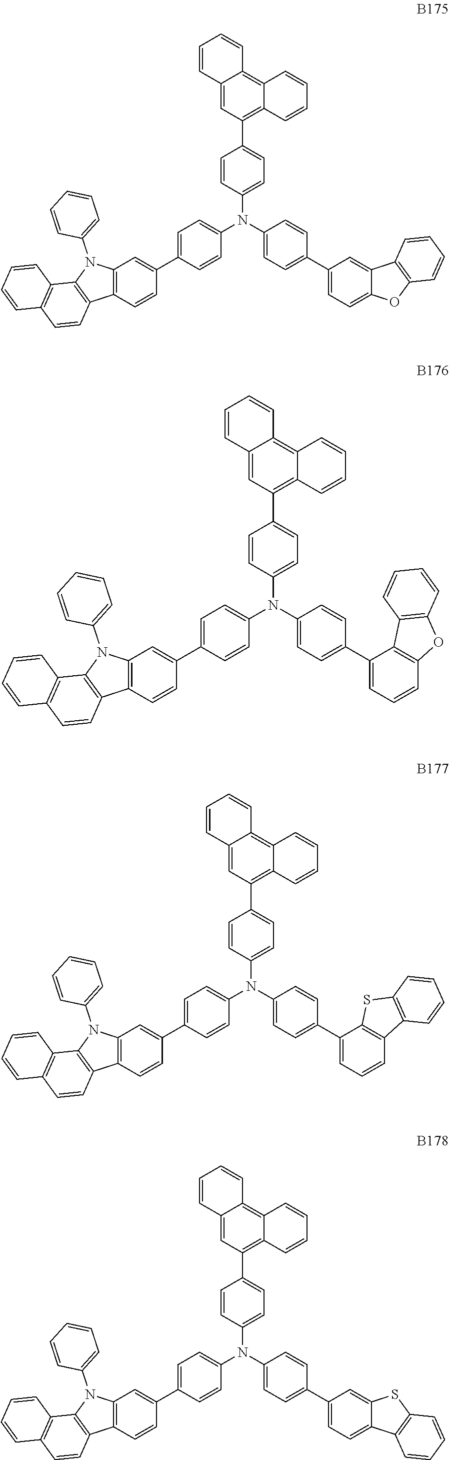 [SMILES: c1ccc(-n2c3cc(-c4ccc(N(c5ccc(-c6ccc7oc8ccccc8c7c6)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6cc7ccccc7c7ccccc67)cc5)c5ccc(-c6cccc7oc8ccccc8c67)cc5)cc4)ccc3c3ccc4ccccc4c32)cc1 ; c1ccc(-n2c3cc(-c4ccc(N(c5ccc(-c6cc7ccccc7c7ccccc67)cc5)c5ccc(-c6cccc7c6sc6ccccc67)cc5)cc4)ccc3c3ccc4ccccc4c32)cc1 ; c1ccc(-n2c3cc(-c4ccc(N(c5ccc(-c6ccc7c(c6)sc6ccccc67)cc5)c5ccc(-c6cc7ccccc7c7ccccc67)cc5)cc4)ccc3c3ccc4ccccc4c32)cc1]

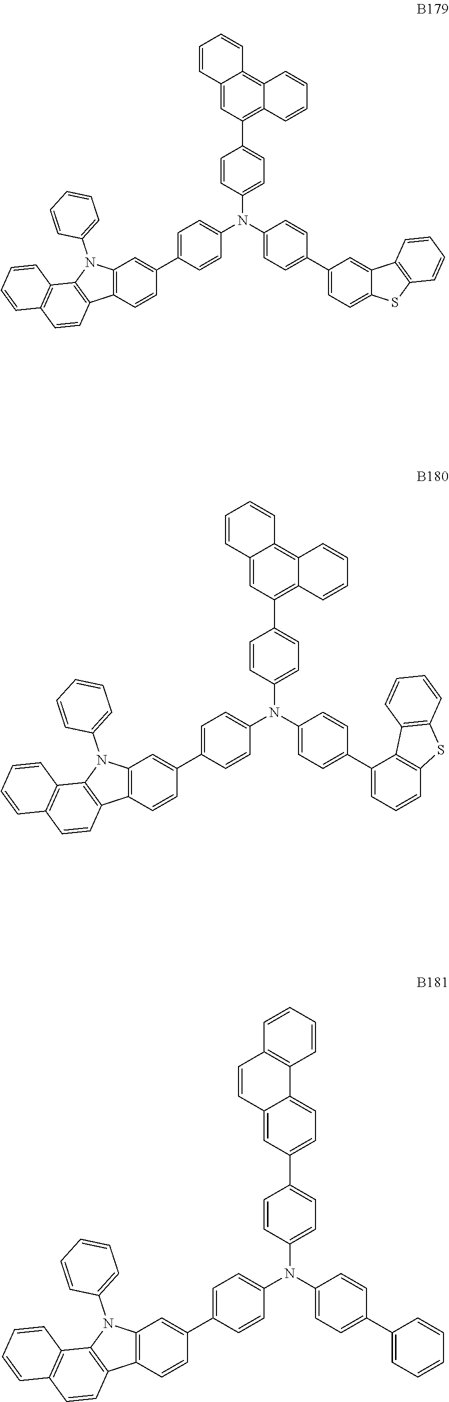 [SMILES: c1ccc(-n2c3cc(-c4ccc(N(c5ccc(-c6ccc7sc8ccccc8c7c6)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6cc7ccccc7c7ccccc67)cc5)c5ccc(-c6cccc7sc8ccccc8c67)cc5)cc4)ccc3c3ccc4ccccc4c32)cc1 ; c1ccc(-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2)cc1]

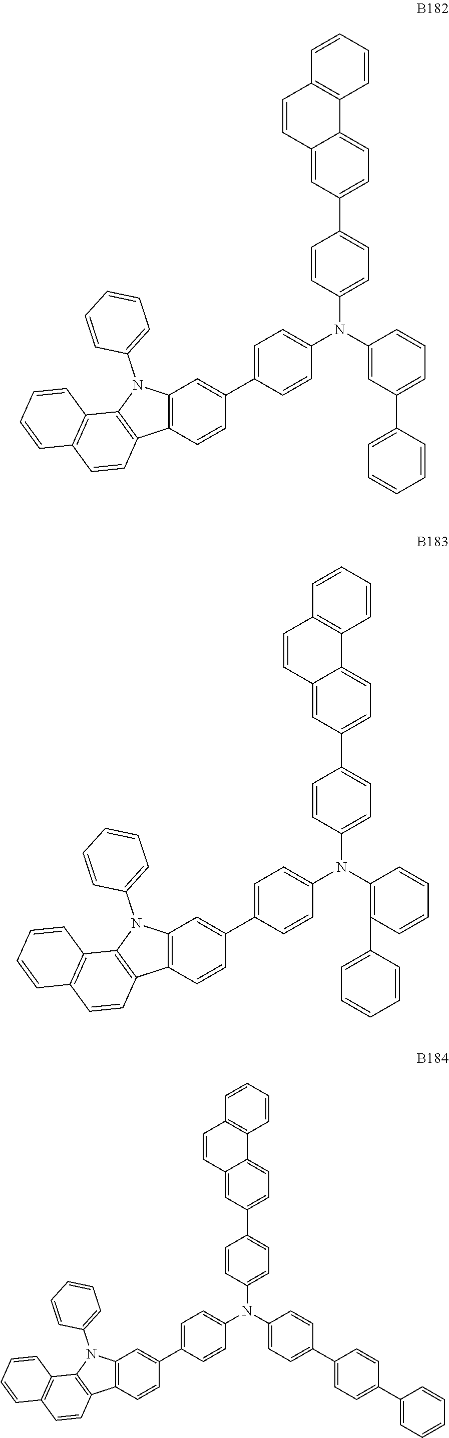 [SMILES: c1ccc(-c2cccc(N(c3ccc(-c4ccc5c(ccc6ccccc65)c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4c(ccc5ccccc54)c3)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6c(ccc7ccccc76)c5)cc4)c4ccc(-c5ccc6c7ccc8ccccc8c7n(-c7ccccc7)c6c5)cc4)cc3)cc2)cc1]

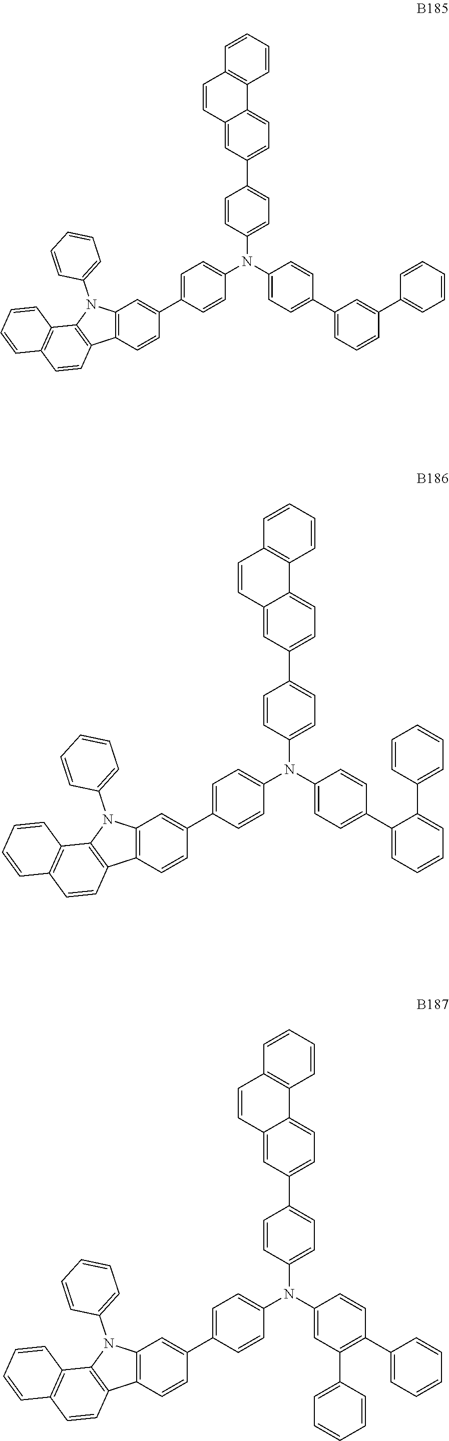 [SMILES: c1ccc(-c2cccc(-c3ccc(N(c4ccc(-c5ccc6c(ccc7ccccc76)c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2)cc1 ; c1ccc(-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2-c2ccccc2)cc1]

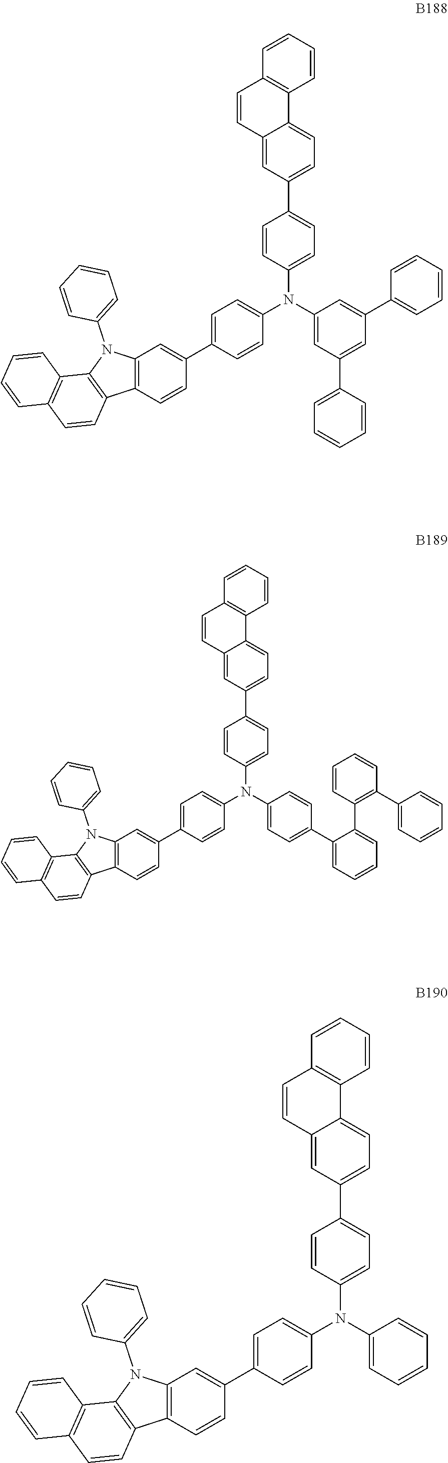 [SMILES: c1ccc(-c2cc(-c3ccccc3)cc(N(c3ccc(-c4ccc5c(ccc6ccccc65)c4)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2)cc1 ; c1ccc(N(c2ccc(-c3ccc4c(ccc5ccccc54)c3)cc2)c2ccc(-c3ccc4c5ccc6ccccc6c5n(-c5ccccc5)c4c3)cc2)cc1]

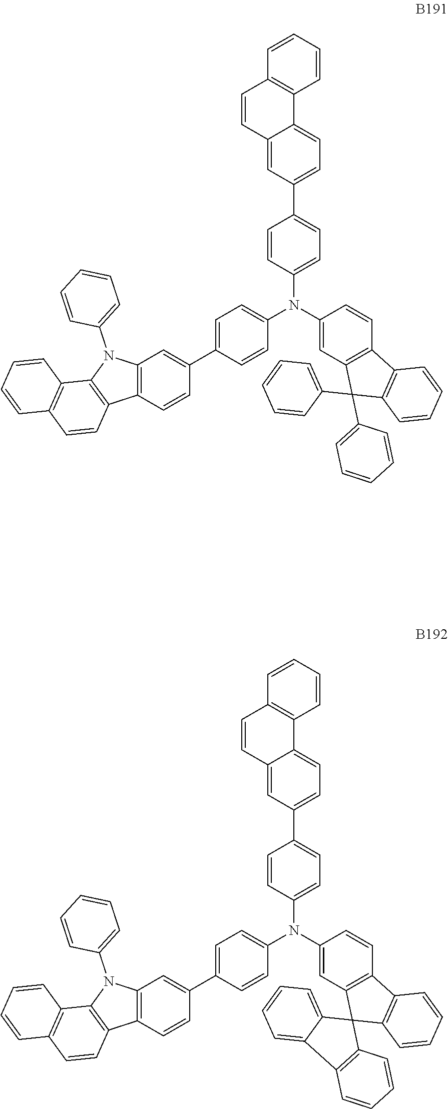 [SMILES: c1ccc(-n2c3cc(-c4ccc(N(c5ccc(-c6ccc7c(ccc8ccccc87)c6)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6c(c5)C5(c7ccccc7-c7ccccc75)c5ccccc5-6)cc4)ccc3c3ccc4ccccc4c32)cc1]

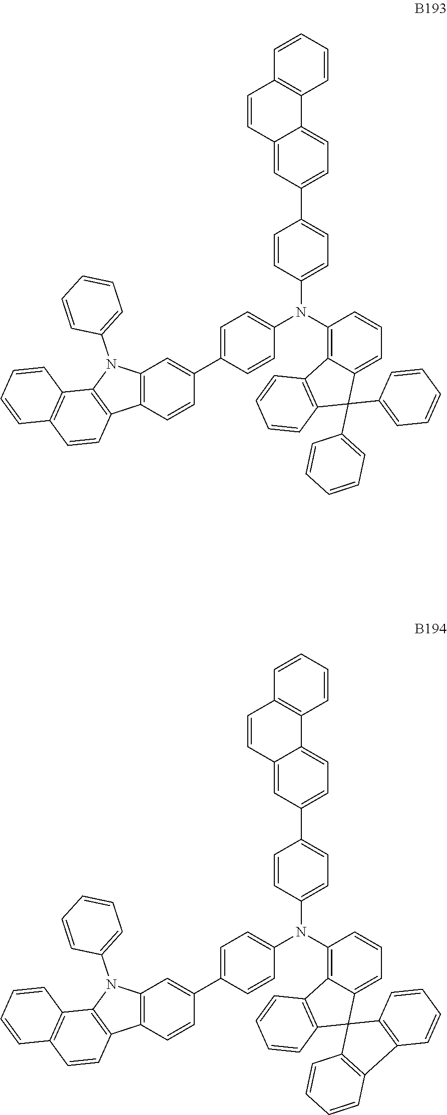 [SMILES: c1ccc(-n2c3cc(-c4ccc(N(c5ccc(-c6ccc7c(ccc8ccccc87)c6)cc5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc(-c6ccc7c(ccc8ccccc87)c6)cc5)c5cccc6c5-c5ccccc5C65c6ccccc6-c6ccccc65)cc4)ccc3c3ccc4ccccc4c32)cc1]

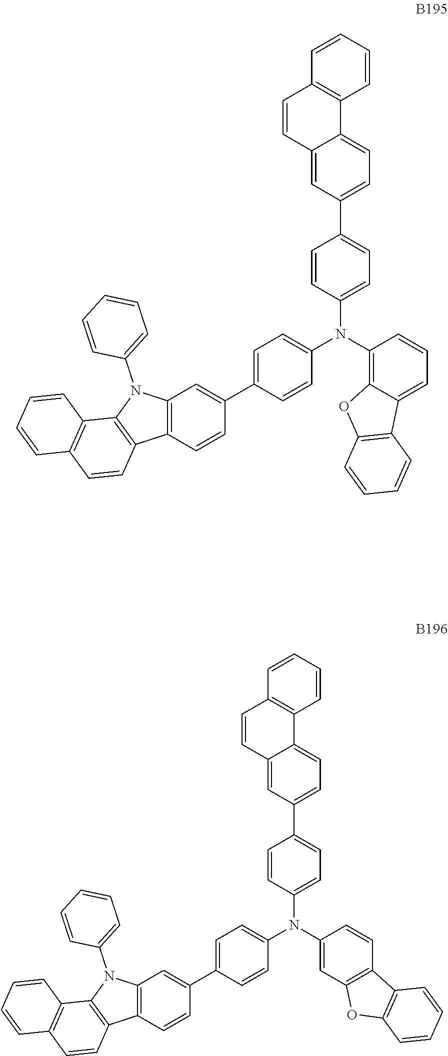 [SMILES: c1ccc(-n2c3cc(-c4ccc(N(c5ccc(-c6ccc7c(ccc8ccccc87)c6)cc5)c5cccc6c5oc5ccccc56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6c(c5)oc5ccccc56)cc4)ccc3c3ccc4ccccc4c32)cc1]

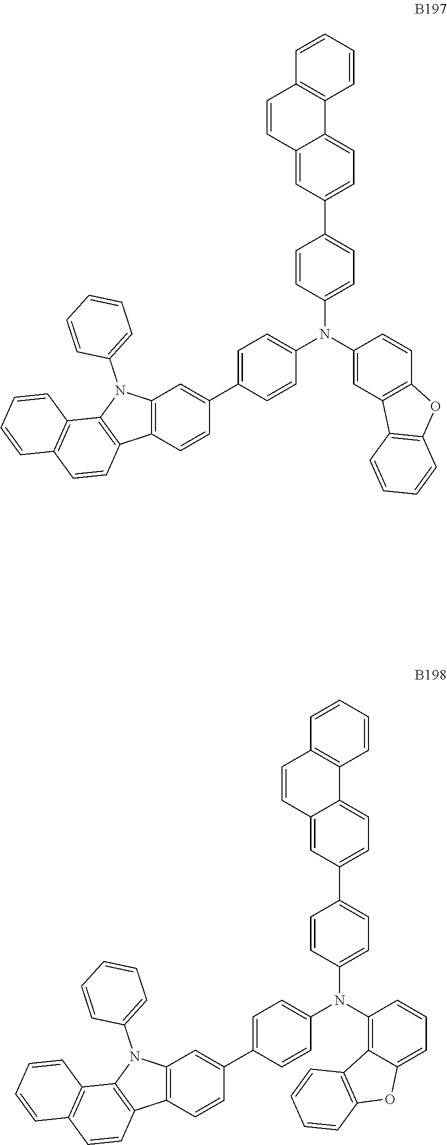 [SMILES: c1ccc(-n2c3cc(-c4ccc(N(c5ccc(-c6ccc7c(ccc8ccccc87)c6)cc5)c5ccc6oc7ccccc7c6c5)cc4)ccc3c3ccc4ccccc4c32)cc1 ; c1ccc(-n2c3cc(-c4ccc(N(c5ccc(-c6ccc7c(ccc8ccccc87)c6)cc5)c5cccc6oc7ccccc7c56)cc4)ccc3c3ccc4ccccc4c32)cc1]

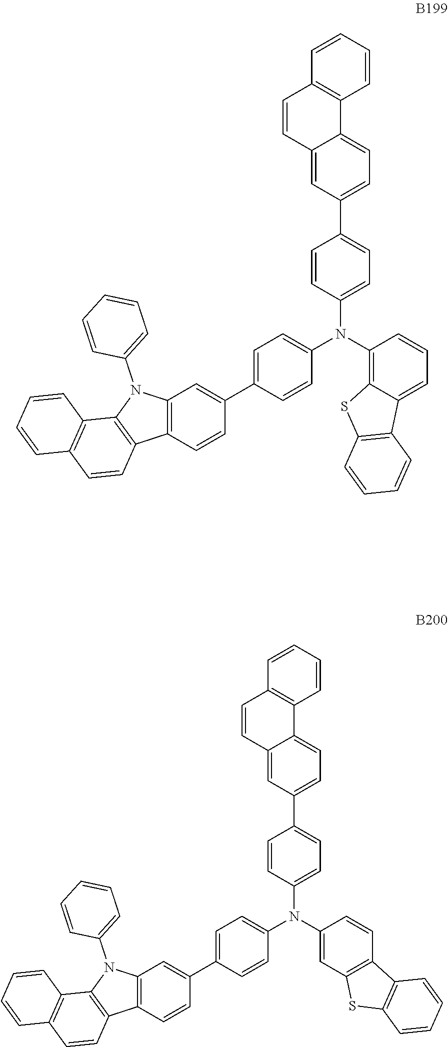 [SMILES: c1ccc(-n2c3cc(-c4ccc(N(c5ccc(-c6ccc7c(ccc8ccccc87)c6)cc5)c5cccc6c5sc5ccccc56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6c(c5)sc5ccccc56)cc4)ccc3c3ccc4ccccc4c32)cc1]

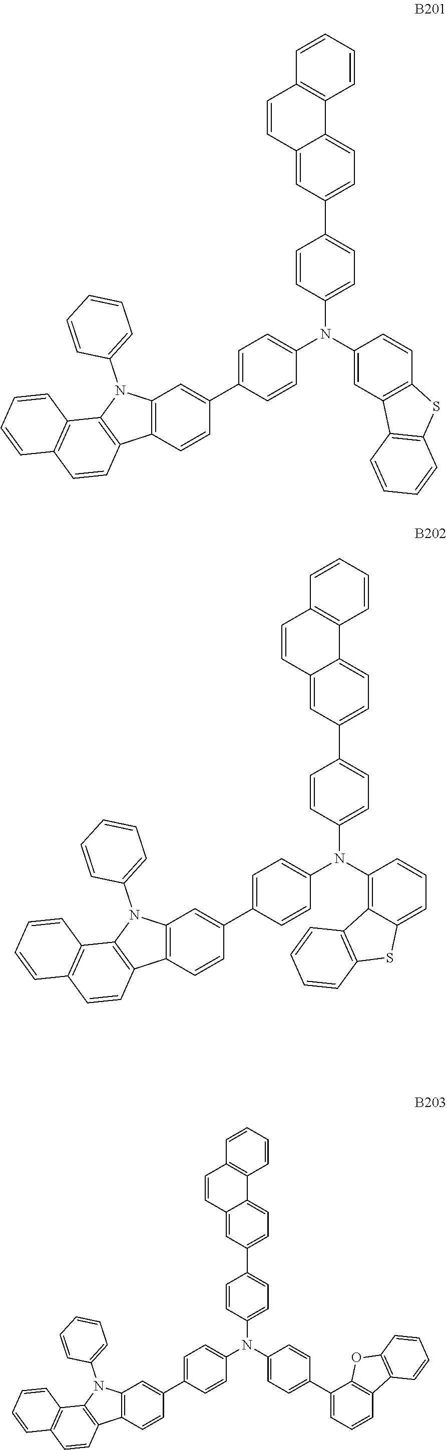 [SMILES: c1ccc(-n2c3cc(-c4ccc(N(c5ccc(-c6ccc7c(ccc8ccccc87)c6)cc5)c5ccc6sc7ccccc7c6c5)cc4)ccc3c3ccc4ccccc4c32)cc1 ; c1ccc(-n2c3cc(-c4ccc(N(c5ccc(-c6ccc7c(ccc8ccccc87)c6)cc5)c5cccc6sc7ccccc7c56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7c6oc6ccccc67)cc5)cc4)ccc3c3ccc4ccccc4c32)cc1]

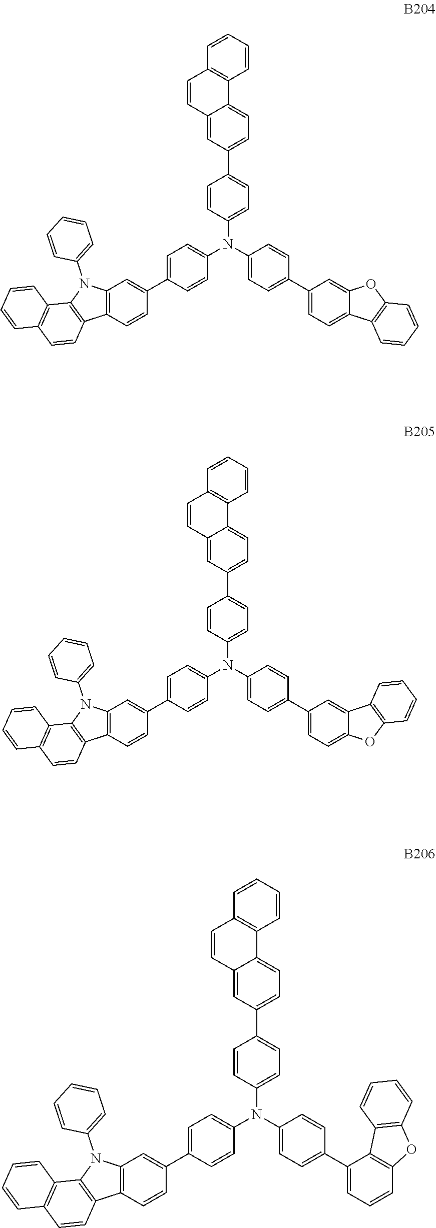 [SMILES: c1ccc(-n2c3cc(-c4ccc(N(c5ccc(-c6ccc7c(ccc8ccccc87)c6)cc5)c5ccc(-c6ccc7c(c6)oc6ccccc67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6ccc7oc8ccccc8c7c6)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7oc8ccccc8c67)cc5)cc4)ccc3c3ccc4ccccc4c32)cc1]

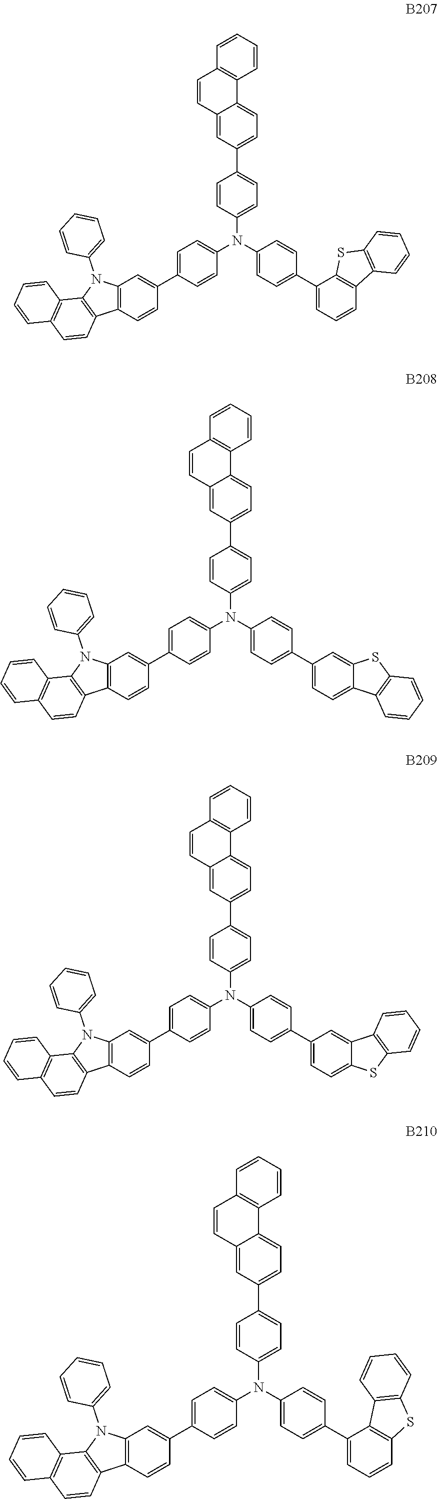 [SMILES: c1ccc(-n2c3cc(-c4ccc(N(c5ccc(-c6ccc7c(ccc8ccccc87)c6)cc5)c5ccc(-c6cccc7c6sc6ccccc67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6ccc7c(c6)sc6ccccc67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6ccc7sc8ccccc8c7c6)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7sc8ccccc8c67)cc5)cc4)ccc3c3ccc4ccccc4c32)cc1]

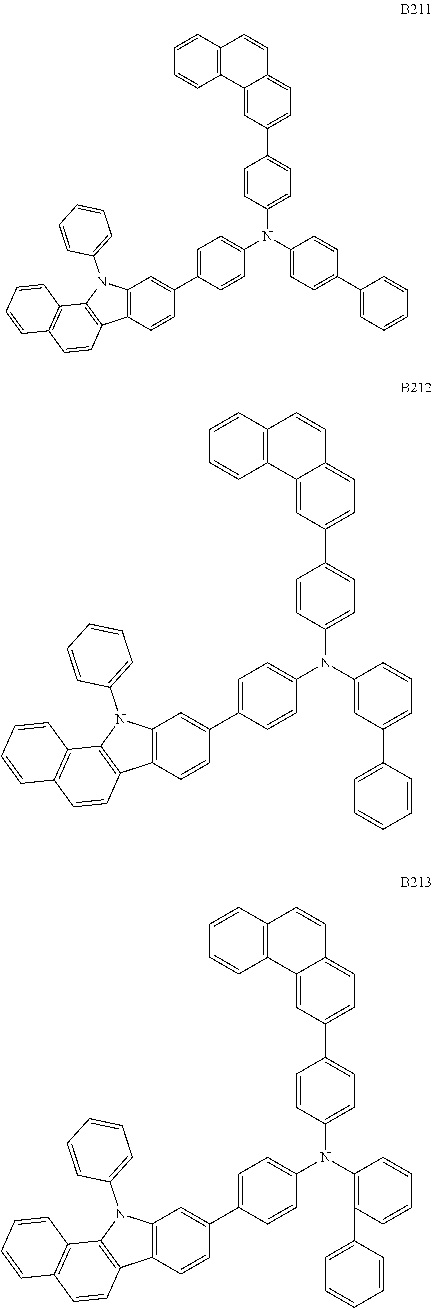 [SMILES: c1ccc(-c2ccc(N(c3ccc(-c4ccc5ccc6ccccc6c5c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5ccc6ccccc6c5c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4ccc5ccccc5c4c3)cc2)c2ccc(-c3ccc4c5ccc6ccccc6c5n(-c5ccccc5)c4c3)cc2)cc1]

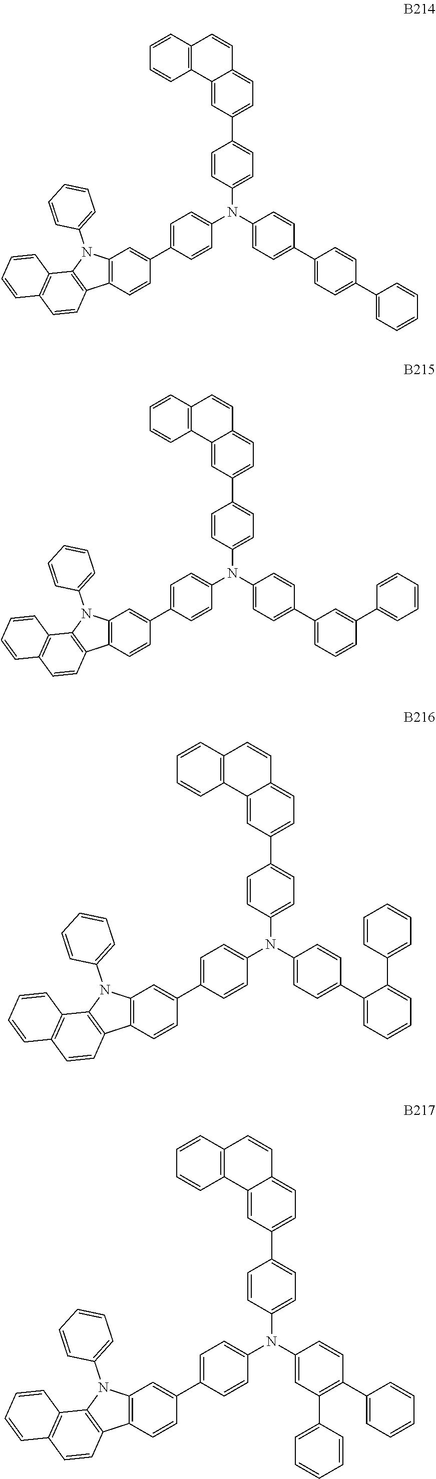 [SMILES: c1ccc(-c2ccc(-c3ccc(N(c4ccc(-c5ccc6ccc7ccccc7c6c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6ccc7ccccc7c6c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5ccc6ccccc6c5c4)cc3)c3ccc(-c4ccc5c6ccc7ccccc7c6n(-c6ccccc6)c5c4)cc3)cc2)cc1 ; c1ccc(-c2ccc(N(c3ccc(-c4ccc5ccc6ccccc6c5c4)cc3)c3ccc(-c4ccc5c6ccc7ccccc7c6n(-c6ccccc6)c5c4)cc3)cc2-c2ccccc2)cc1]

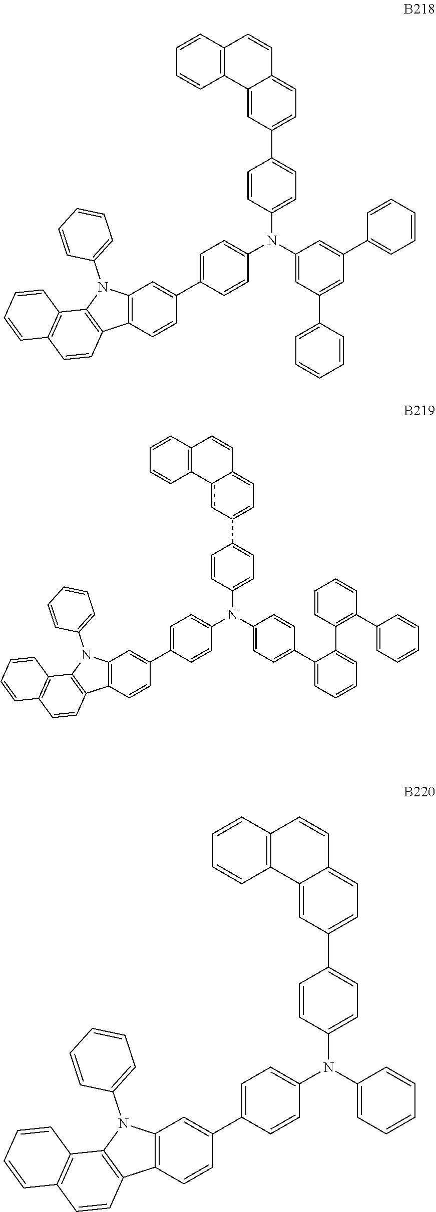

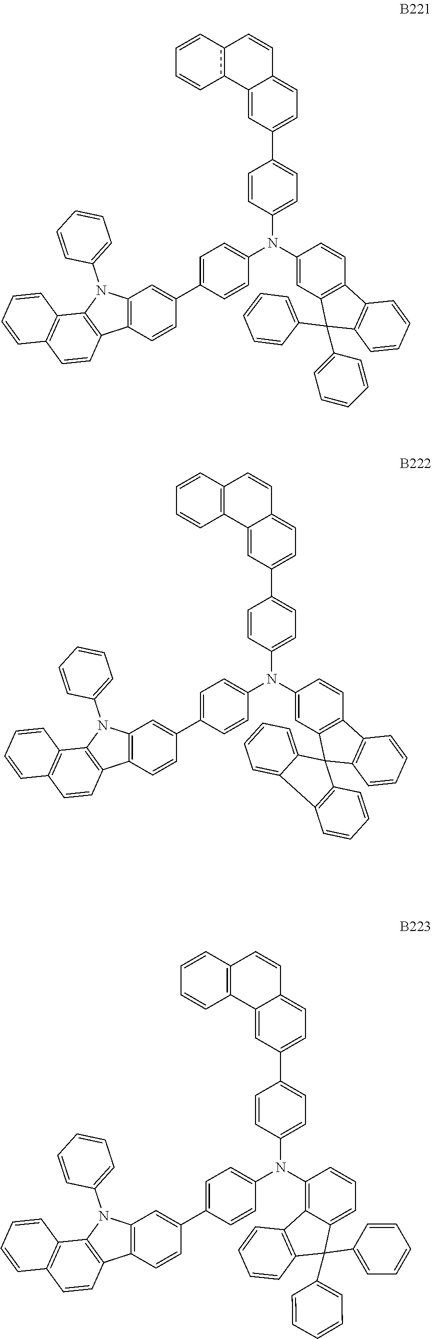

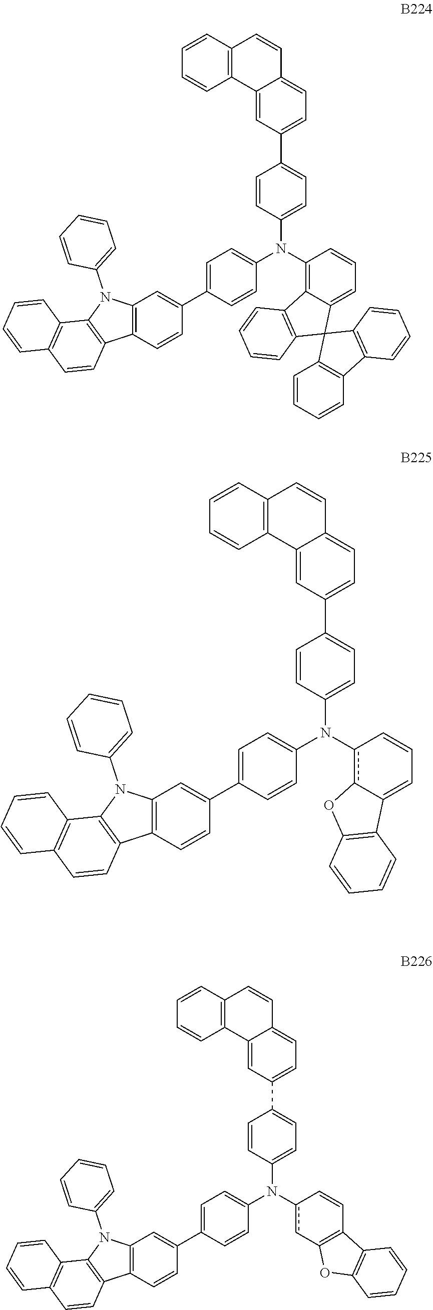

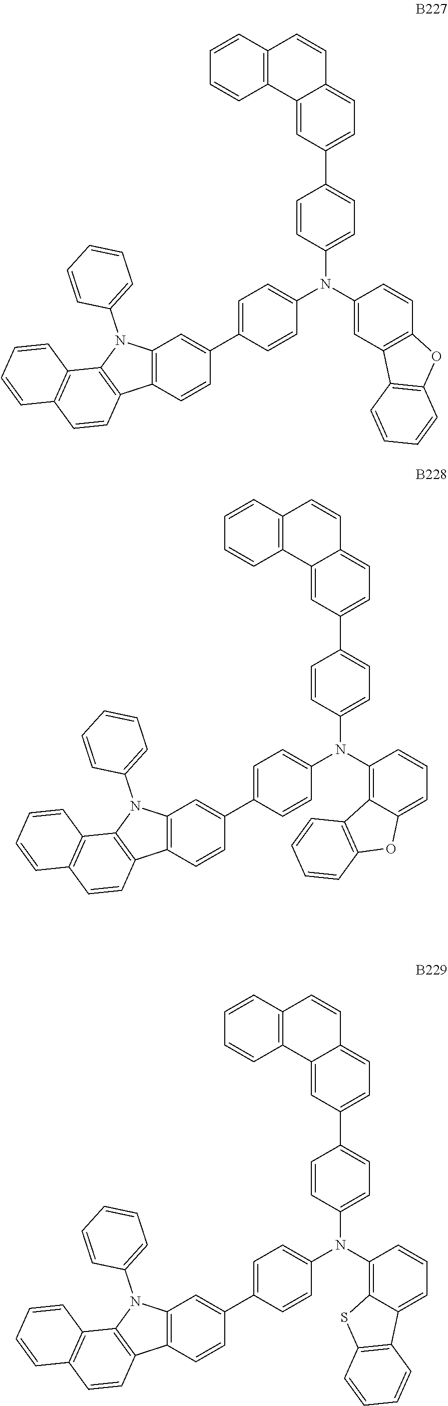 [SMILES: c1ccc(-n2c3cc(-c4ccc(N(c5ccc(-c6ccc7ccc8ccccc8c7c6)cc5)c5ccc6oc7ccccc7c6c5)cc4)ccc3c3ccc4ccccc4c32)cc1 ; c1ccc(-n2c3cc(-c4ccc(N(c5ccc(-c6ccc7ccc8ccccc8c7c6)cc5)c5cccc6oc7ccccc7c56)cc4)ccc3c3ccc4ccccc4c32)cc1 ; c1ccc(-n2c3cc(-c4ccc(N(c5ccc(-c6ccc7ccc8ccccc8c7c6)cc5)c5cccc6c5sc5ccccc56)cc4)ccc3c3ccc4ccccc4c32)cc1]

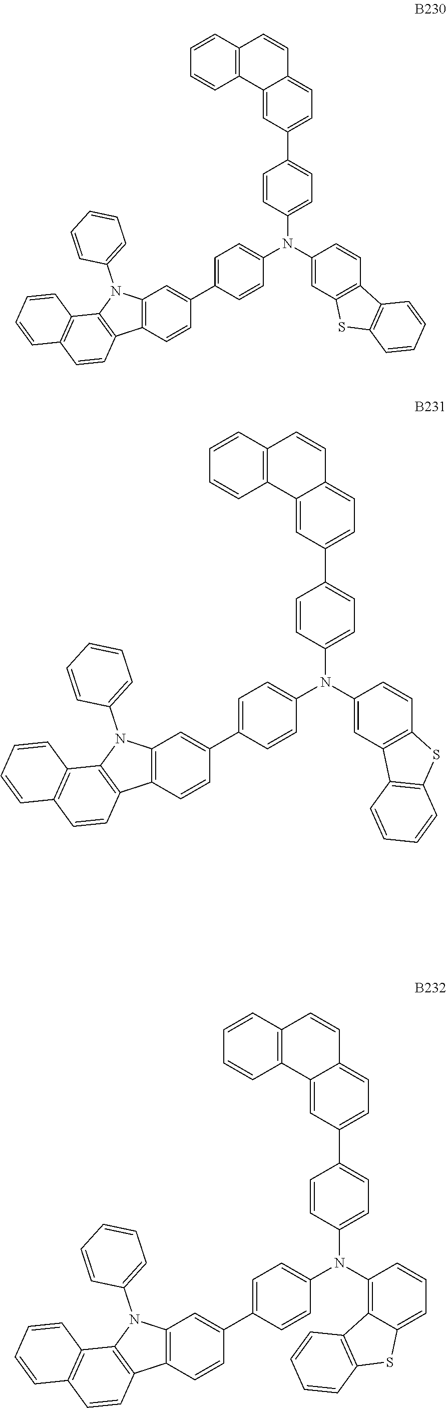 [SMILES: c1ccc(-n2c3cc(-c4ccc(N(c5ccc(-c6ccc7ccc8ccccc8c7c6)cc5)c5ccc6c(c5)sc5ccccc56)cc4)ccc3c3ccc4ccccc4c32)cc1 ; c1ccc(-n2c3cc(-c4ccc(N(c5ccc(-c6ccc7ccc8ccccc8c7c6)cc5)c5ccc6sc7ccccc7c6c5)cc4)ccc3c3ccc4ccccc4c32)cc1 ; c1ccc(-n2c3cc(-c4ccc(N(c5ccc(-c6ccc7ccc8ccccc8c7c6)cc5)c5cccc6sc7ccccc7c56)cc4)ccc3c3ccc4ccccc4c32)cc1]

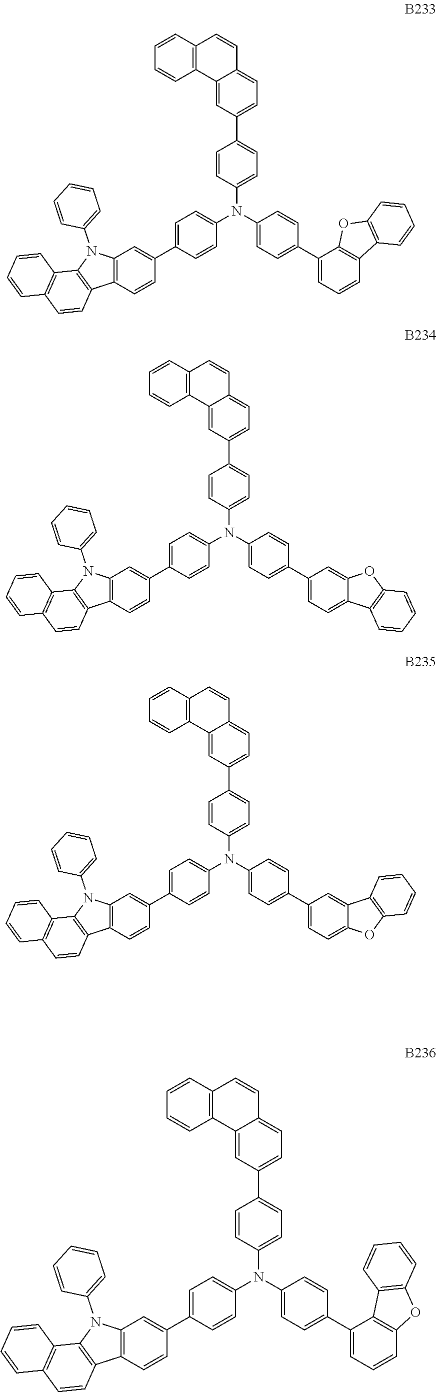 [SMILES: c1ccc(-n2c3cc(-c4ccc(N(c5ccc(-c6ccc7ccc8ccccc8c7c6)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7c(c6)oc6ccccc67)cc5)c5ccc(-c6ccc7ccc8ccccc8c7c6)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6ccc7oc8ccccc8c7c6)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6cccc7oc8ccccc8c67)cc5)cc4)ccc3c3ccc4ccccc4c32)cc1]

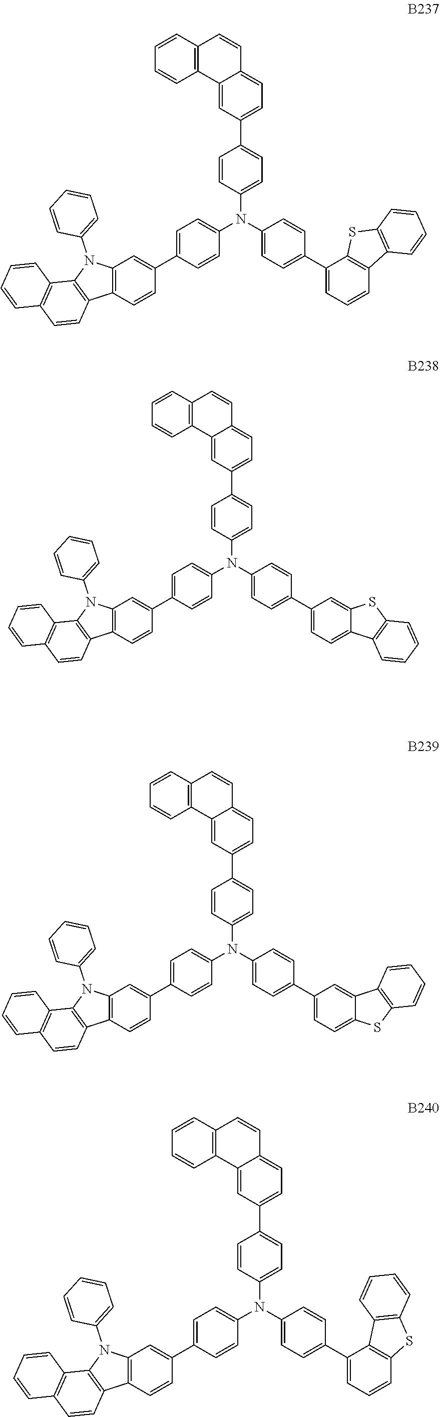 [SMILES: c1ccc(-n2c3cc(-c4ccc(N(c5ccc(-c6ccc7ccc8ccccc8c7c6)cc5)c5ccc(-c6cccc7c6sc6ccccc67)cc5)cc4)ccc3c3ccc4ccccc4c32)cc1 ; c1ccc(-n2c3cc(-c4ccc(N(c5ccc(-c6ccc7c(c6)sc6ccccc67)cc5)c5ccc(-c6ccc7ccc8ccccc8c7c6)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6ccc7sc8ccccc8c7c6)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6cccc7sc8ccccc8c67)cc5)cc4)ccc3c3ccc4ccccc4c32)cc1]

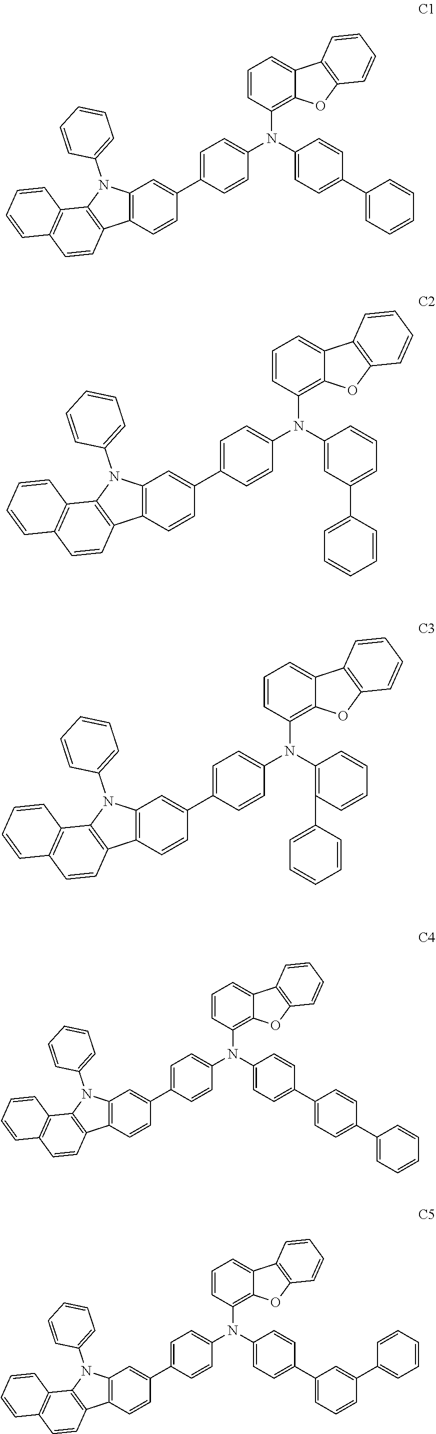 [SMILES: c1ccc(-c2ccc(N(c3ccc(-c4ccc5c6ccc7ccccc7c6n(-c6ccccc6)c5c4)cc3)c3cccc4c3oc3ccccc34)cc2)cc1 ; c1ccc(-c2cccc(N(c3ccc(-c4ccc5c6ccc7ccccc7c6n(-c6ccccc6)c5c4)cc3)c3cccc4c3oc3ccccc34)c2)cc1 ; c1ccc(-c2ccccc2N(c2ccc(-c3ccc4c5ccc6ccccc6c5n(-c5ccccc5)c4c3)cc2)c2cccc3c2oc2ccccc23)cc1 ; c1ccc(-c2ccc(-c3ccc(N(c4ccc(-c5ccc6c7ccc8ccccc8c7n(-c7ccccc7)c6c5)cc4)c4cccc5c4oc4ccccc45)cc3)cc2)cc1 ; c1ccc(-c2cccc(-c3ccc(N(c4ccc(-c5ccc6c7ccc8ccccc8c7n(-c7ccccc7)c6c5)cc4)c4cccc5c4oc4ccccc45)cc3)c2)cc1]

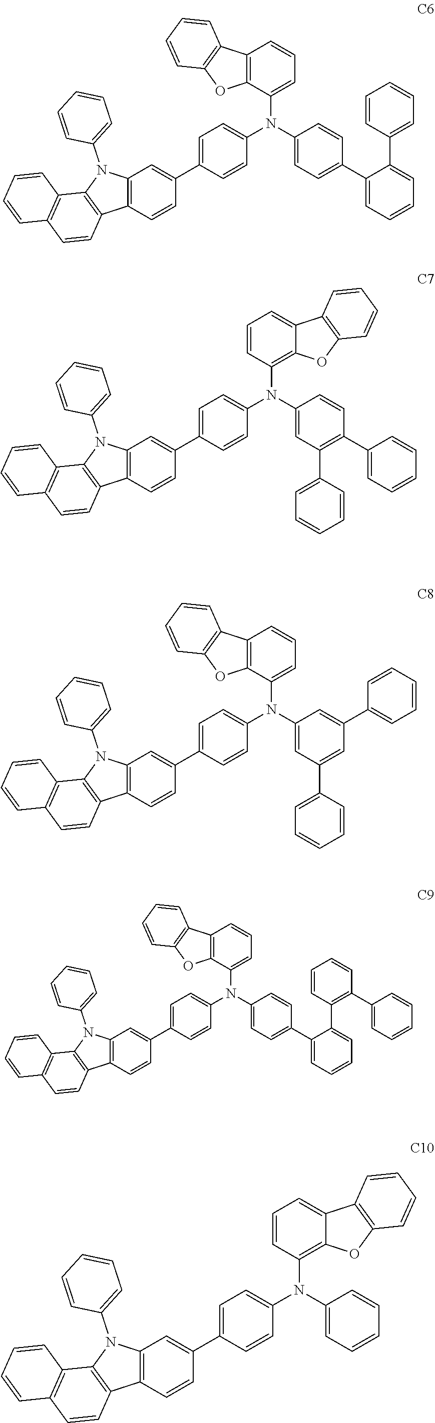 [SMILES: c1ccc(-c2ccccc2-c2ccc(N(c3ccc(-c4ccc5c6ccc7ccccc7c6n(-c6ccccc6)c5c4)cc3)c3cccc4c3oc3ccccc34)cc2)cc1 ; c1ccc(-c2ccc(N(c3ccc(-c4ccc5c6ccc7ccccc7c6n(-c6ccccc6)c5c4)cc3)c3cccc4c3oc3ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4c3oc3ccccc34)c2)cc1 ; c1ccc(-c2ccccc2-c2ccccc2-c2ccc(N(c3ccc(-c4ccc5c6ccc7ccccc7c6n(-c6ccccc6)c5c4)cc3)c3cccc4c3oc3ccccc34)cc2)cc1 ; c1ccc(N(c2ccc(-c3ccc4c5ccc6ccccc6c5n(-c5ccccc5)c4c3)cc2)c2cccc3c2oc2ccccc23)cc1]

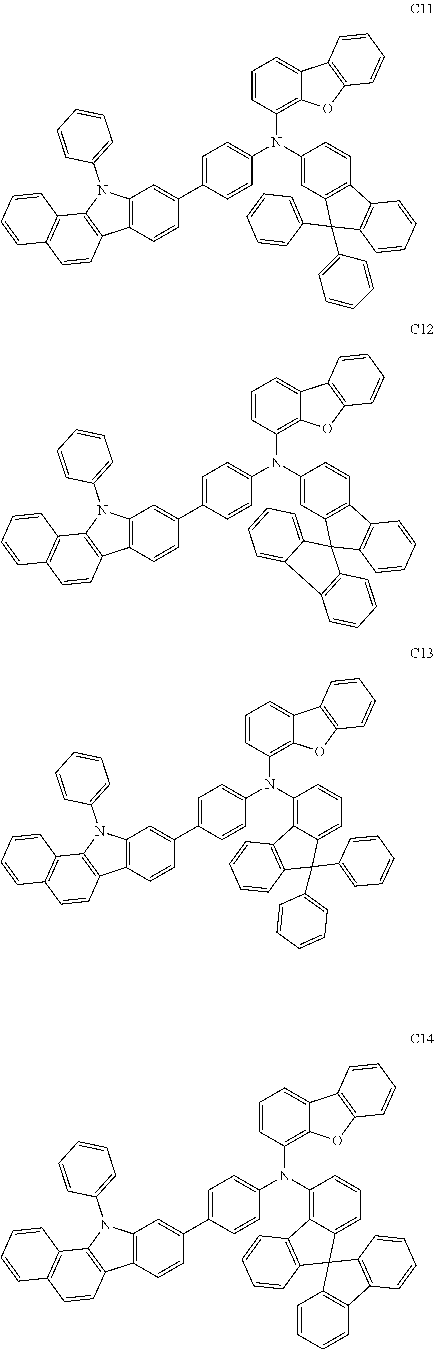 [SMILES: c1ccc(-n2c3cc(-c4ccc(N(c5ccc6c(c5)C(c5ccccc5)(c5ccccc5)c5ccccc5-6)c5cccc6c5oc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5cccc6c5oc5ccccc56)cc4)ccc3c3ccc4ccccc4c32)cc1 ; c1ccc(-n2c3cc(-c4ccc(N(c5cccc6c5-c5ccccc5C6(c5ccccc5)c5ccccc5)c5cccc6c5oc5ccccc56)cc4)ccc3c3ccc4ccccc4c32)cc1 ; c1ccc(-n2c3cc(-c4ccc(N(c5cccc6c5-c5ccccc5C65c6ccccc6-c6ccccc65)c5cccc6c5oc5ccccc56)cc4)ccc3c3ccc4ccccc4c32)cc1]

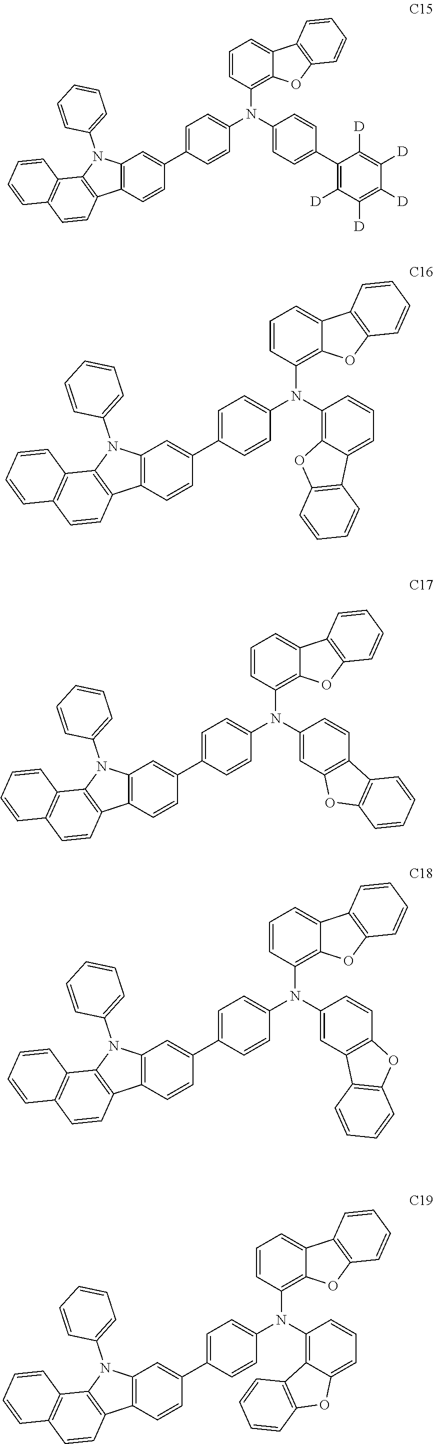 [SMILES: [2H]c1c([2H])c([2H])c(-c2ccc(N(c3ccc(-c4ccc5c6ccc7ccccc7c6n(-c6ccccc6)c5c4)cc3)c3cccc4c3oc3ccccc34)cc2)c([2H])c1[2H] ; c1ccc(-n2c3cc(-c4ccc(N(c5cccc6c5oc5ccccc56)c5cccc6c5oc5ccccc56)cc4)ccc3c3ccc4ccccc4c32)cc1 ; c1ccc(-n2c3cc(-c4ccc(N(c5ccc6c(c5)oc5ccccc56)c5cccc6c5oc5ccccc56)cc4)ccc3c3ccc4ccccc4c32)cc1 ; c1ccc(-n2c3cc(-c4ccc(N(c5ccc6oc7ccccc7c6c5)c5cccc6c5oc5ccccc56)cc4)ccc3c3ccc4ccccc4c32)cc1 ; c1ccc(-n2c3cc(-c4ccc(N(c5cccc6c5oc5ccccc56)c5cccc6oc7ccccc7c56)cc4)ccc3c3ccc4ccccc4c32)cc1]

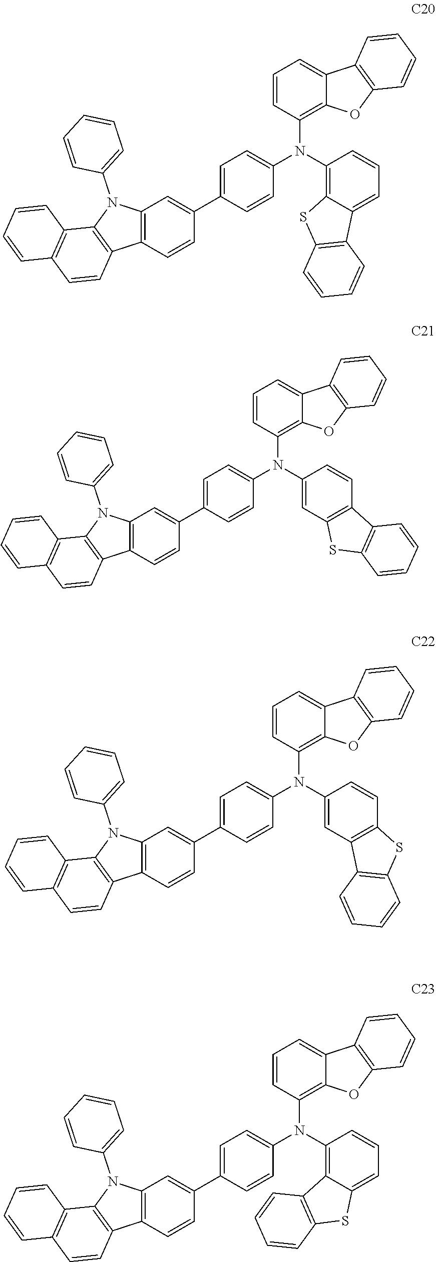 [SMILES: c1ccc(-n2c3cc(-c4ccc(N(c5cccc6c5oc5ccccc56)c5cccc6c5sc5ccccc56)cc4)ccc3c3ccc4ccccc4c32)cc1 ; c1ccc(-n2c3cc(-c4ccc(N(c5ccc6c(c5)sc5ccccc56)c5cccc6c5oc5ccccc56)cc4)ccc3c3ccc4ccccc4c32)cc1 ; c1ccc(-n2c3cc(-c4ccc(N(c5ccc6sc7ccccc7c6c5)c5cccc6c5oc5ccccc56)cc4)ccc3c3ccc4ccccc4c32)cc1 ; c1ccc(-n2c3cc(-c4ccc(N(c5cccc6c5oc5ccccc56)c5cccc6sc7ccccc7c56)cc4)ccc3c3ccc4ccccc4c32)cc1]

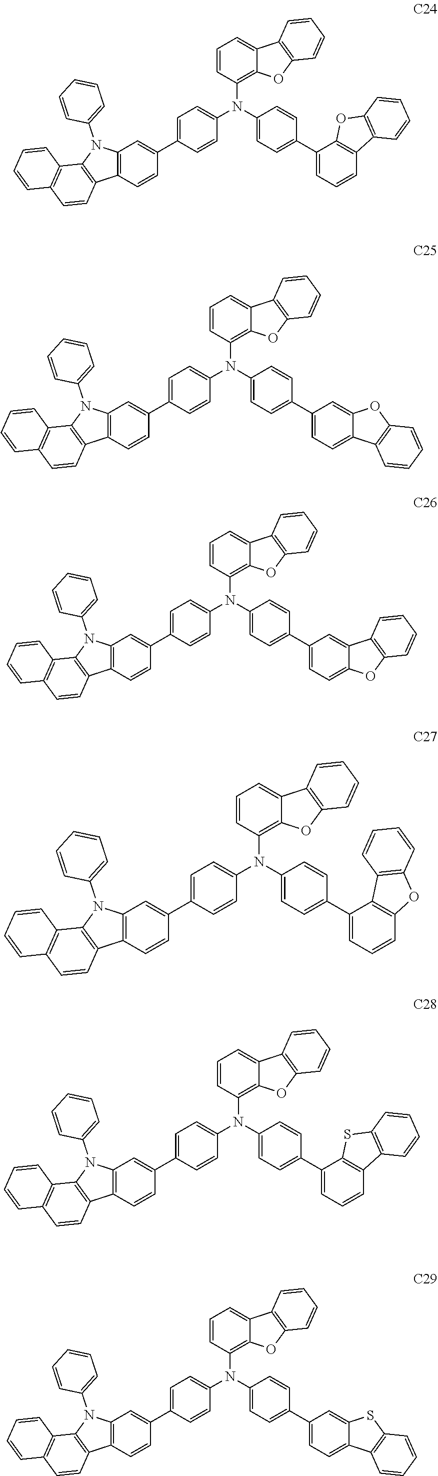

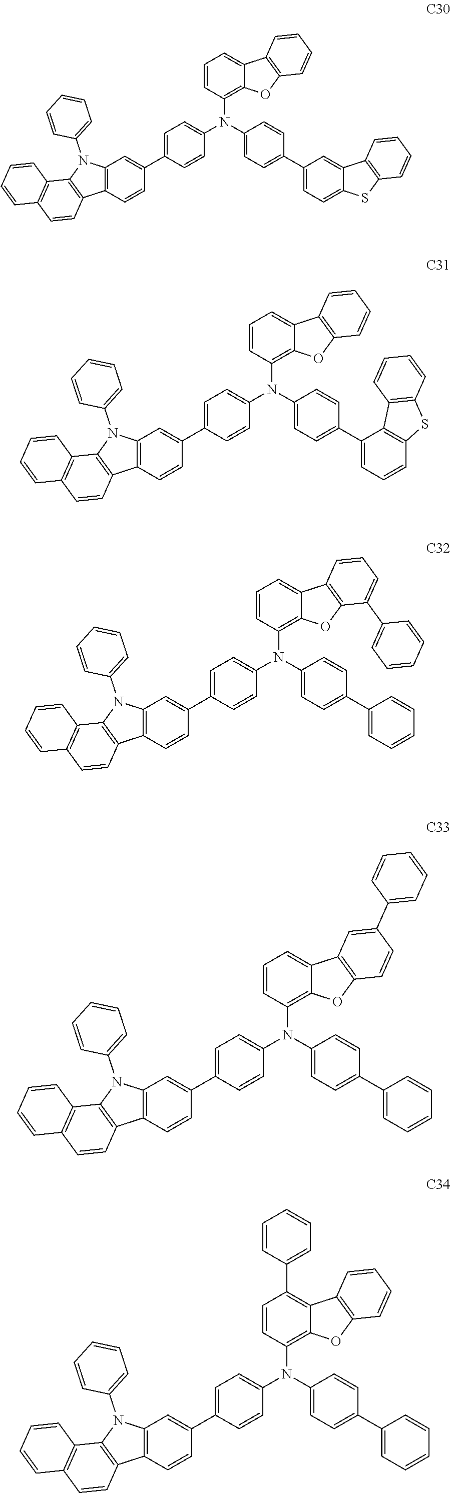

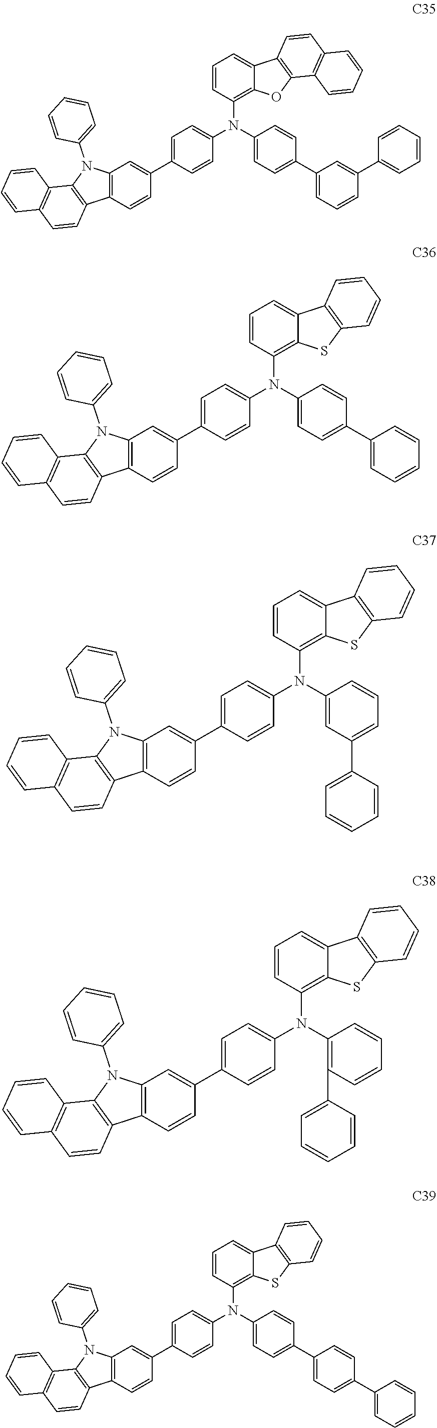 [SMILES: c1ccc(-c2cccc(-c3ccc(N(c4ccc(-c5ccc6c7ccc8ccccc8c7n(-c7ccccc7)c6c5)cc4)c4cccc5c4oc4c6ccccc6ccc54)cc3)c2)cc1 ; c1ccc(-c2ccc(N(c3ccc(-c4ccc5c6ccc7ccccc7c6n(-c6ccccc6)c5c4)cc3)c3cccc4c3sc3ccccc34)cc2)cc1 ; c1ccc(-c2cccc(N(c3ccc(-c4ccc5c6ccc7ccccc7c6n(-c6ccccc6)c5c4)cc3)c3cccc4c3sc3ccccc34)c2)cc1 ; c1ccc(-c2ccccc2N(c2ccc(-c3ccc4c5ccc6ccccc6c5n(-c5ccccc5)c4c3)cc2)c2cccc3c2sc2ccccc23)cc1 ; c1ccc(-c2ccc(-c3ccc(N(c4ccc(-c5ccc6c7ccc8ccccc8c7n(-c7ccccc7)c6c5)cc4)c4cccc5c4sc4ccccc45)cc3)cc2)cc1]

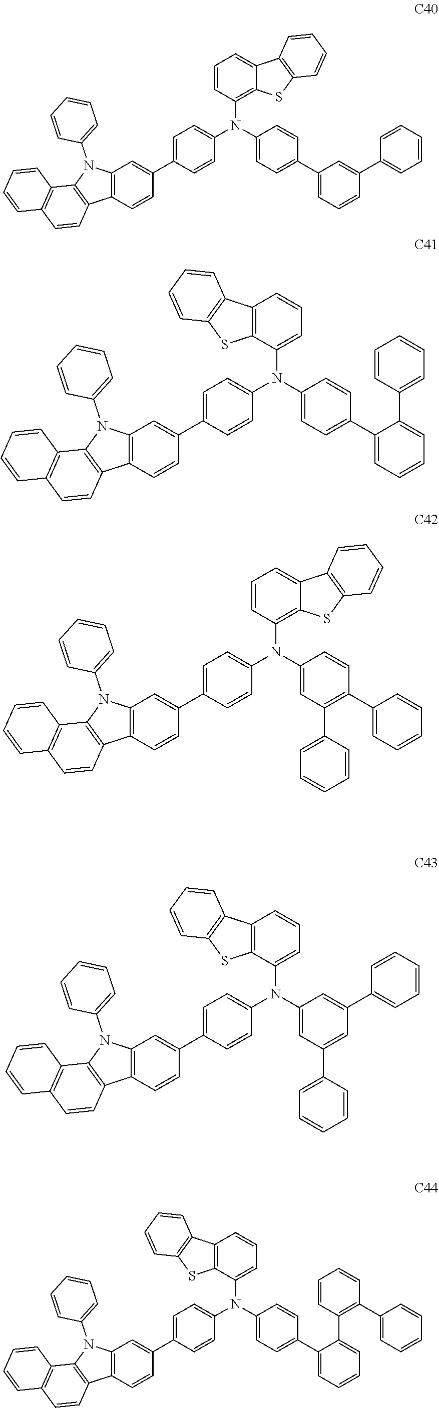

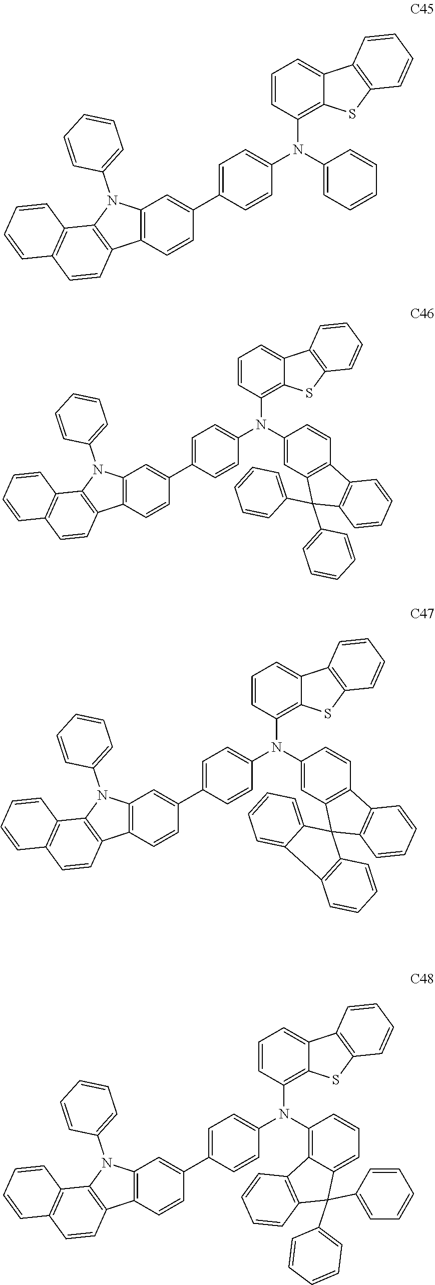 [SMILES: c1ccc(N(c2ccc(-c3ccc4c5ccc6ccccc6c5n(-c5ccccc5)c4c3)cc2)c2cccc3c2sc2ccccc23)cc1 ; c1ccc(-n2c3cc(-c4ccc(N(c5ccc6c(c5)C(c5ccccc5)(c5ccccc5)c5ccccc5-6)c5cccc6c5sc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5cccc6c5sc5ccccc56)cc4)ccc3c3ccc4ccccc4c32)cc1 ; c1ccc(-n2c3cc(-c4ccc(N(c5cccc6c5-c5ccccc5C6(c5ccccc5)c5ccccc5)c5cccc6c5sc5ccccc56)cc4)ccc3c3ccc4ccccc4c32)cc1]

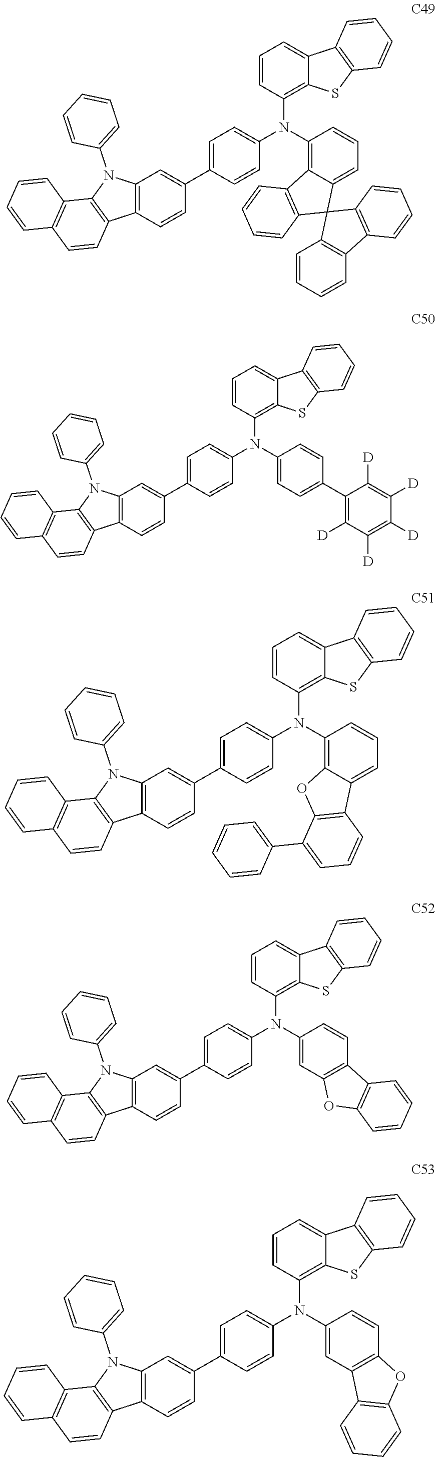

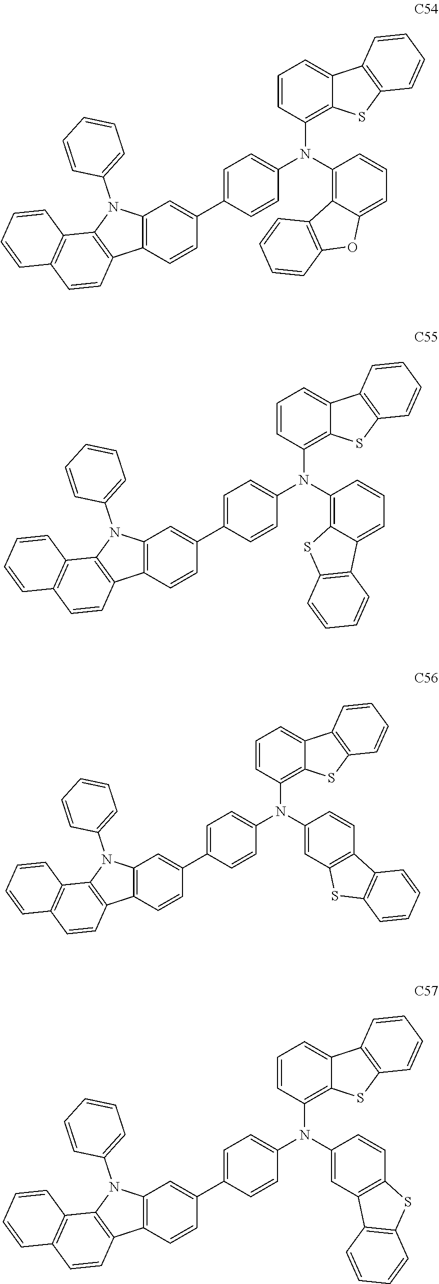 [SMILES: c1ccc(-n2c3cc(-c4ccc(N(c5cccc6c5sc5ccccc56)c5cccc6oc7ccccc7c56)cc4)ccc3c3ccc4ccccc4c32)cc1 ; c1ccc(-n2c3cc(-c4ccc(N(c5cccc6c5sc5ccccc56)c5cccc6c5sc5ccccc56)cc4)ccc3c3ccc4ccccc4c32)cc1 ; c1ccc(-n2c3cc(-c4ccc(N(c5ccc6c(c5)sc5ccccc56)c5cccc6c5sc5ccccc56)cc4)ccc3c3ccc4ccccc4c32)cc1 ; c1ccc(-n2c3cc(-c4ccc(N(c5ccc6sc7ccccc7c6c5)c5cccc6c5sc5ccccc56)cc4)ccc3c3ccc4ccccc4c32)cc1]

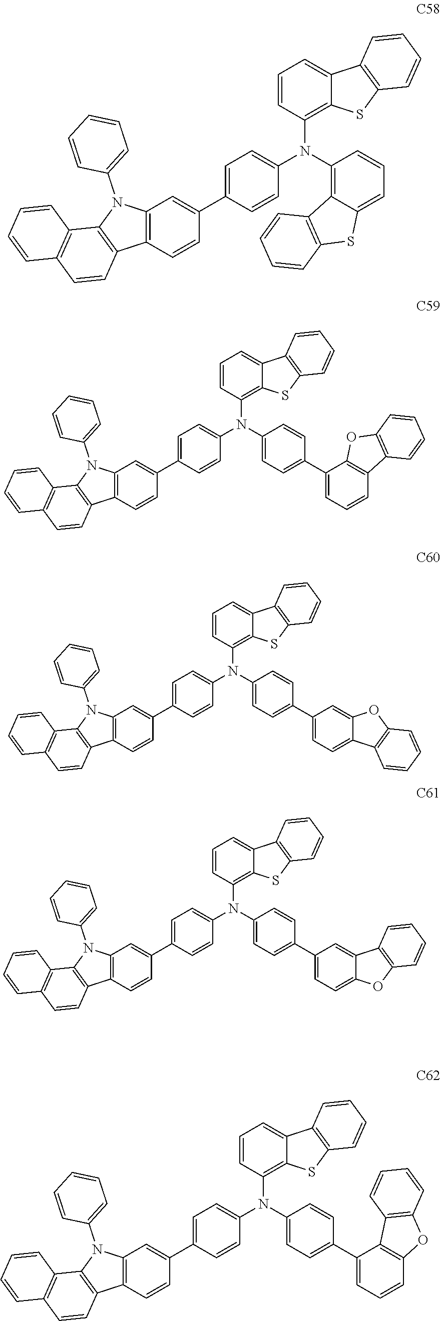

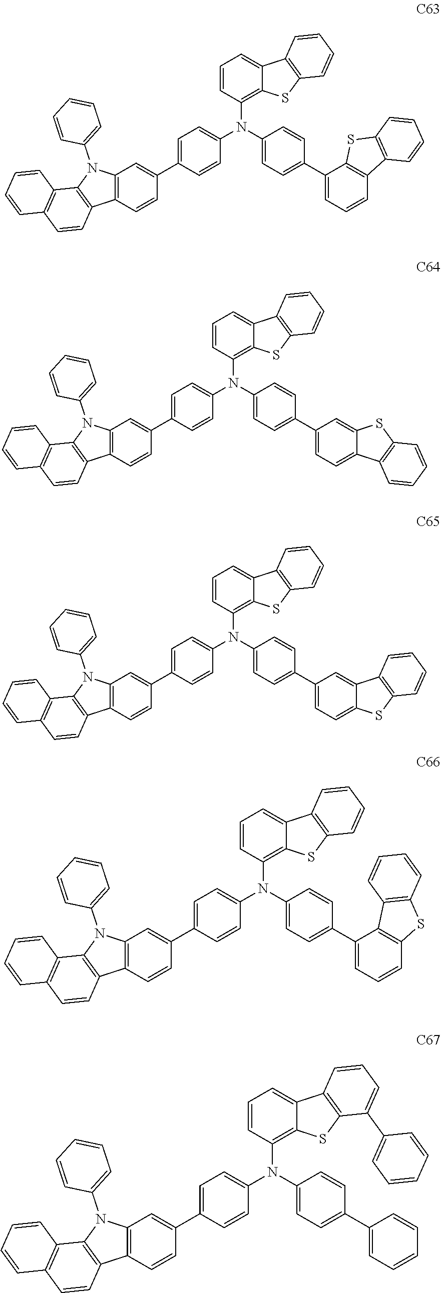

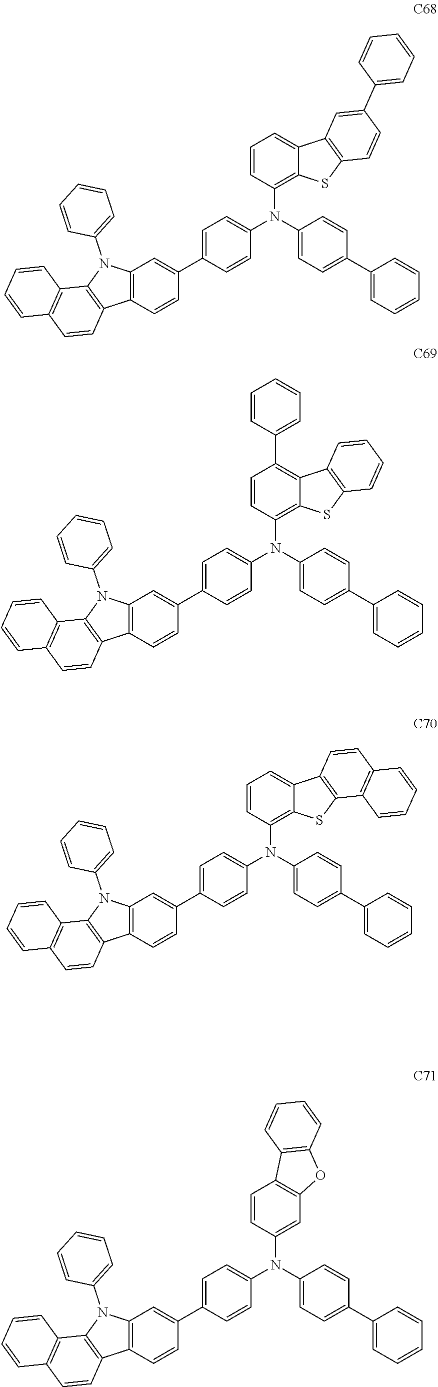 [SMILES: c1ccc(-c2ccc(N(c3ccc(-c4ccc5c6ccc7ccccc7c6n(-c6ccccc6)c5c4)cc3)c3cccc4c3sc3ccc(-c5ccccc5)cc34)cc2)cc1 ; c1ccc(-c2ccc(N(c3ccc(-c4ccc5c6ccc7ccccc7c6n(-c6ccccc6)c5c4)cc3)c3ccc(-c4ccccc4)c4c3sc3ccccc34)cc2)cc1 ; c1ccc(-c2ccc(N(c3ccc(-c4ccc5c6ccc7ccccc7c6n(-c6ccccc6)c5c4)cc3)c3cccc4c3sc3c5ccccc5ccc43)cc2)cc1 ; c1ccc(-c2ccc(N(c3ccc(-c4ccc5c6ccc7ccccc7c6n(-c6ccccc6)c5c4)cc3)c3ccc4c(c3)oc3ccccc34)cc2)cc1]

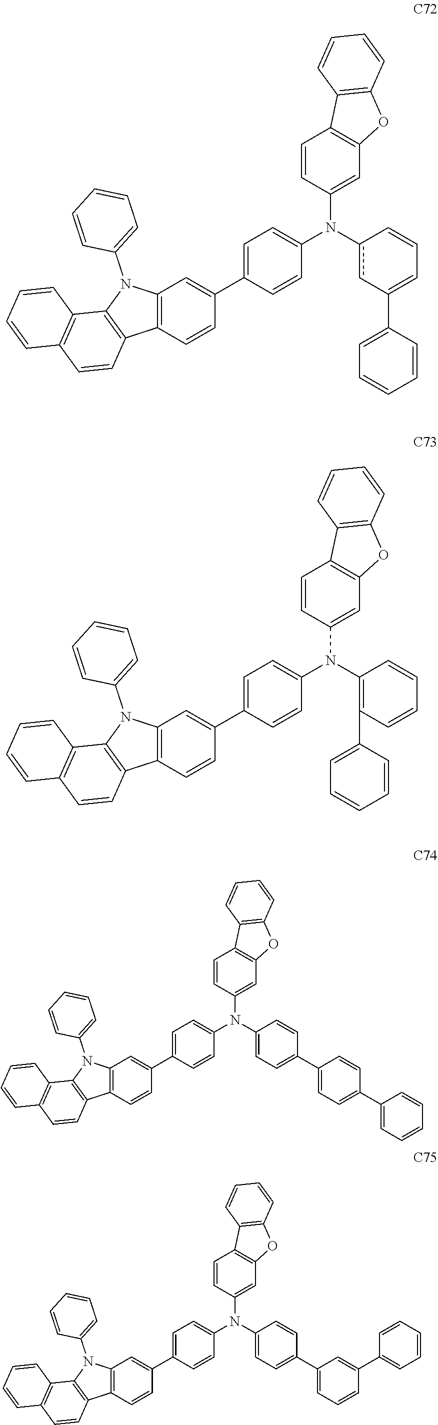

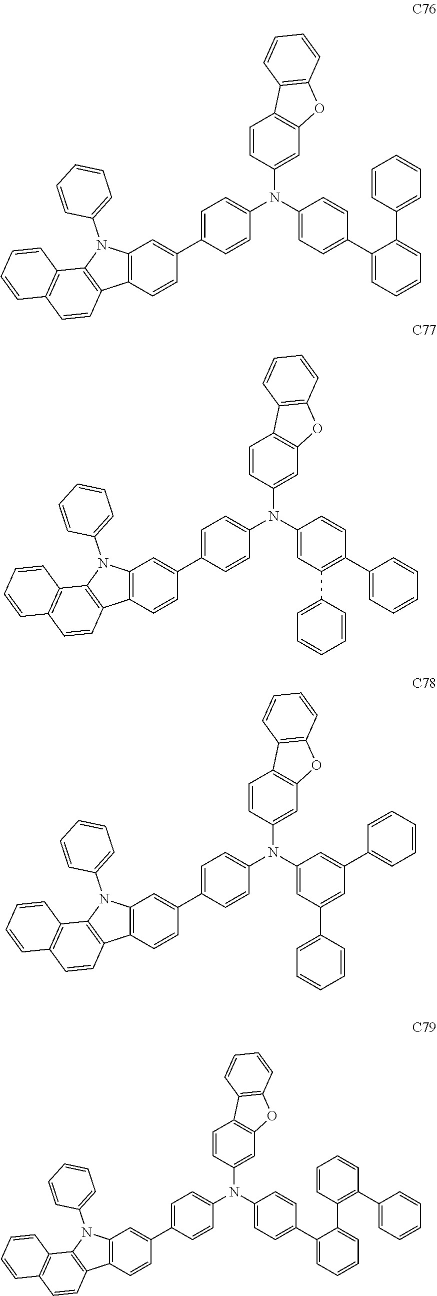

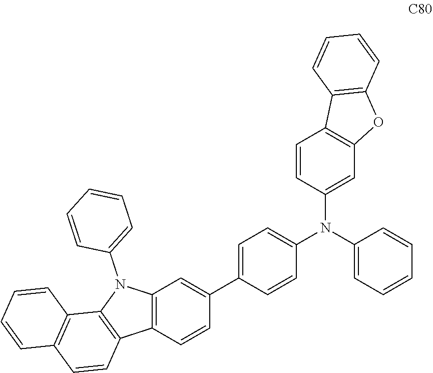 [SMILES: c1ccc(N(c2ccc(-c3ccc4c5ccc6ccccc6c5n(-c5ccccc5)c4c3)cc2)c2ccc3c(c2)oc2ccccc23)cc1]

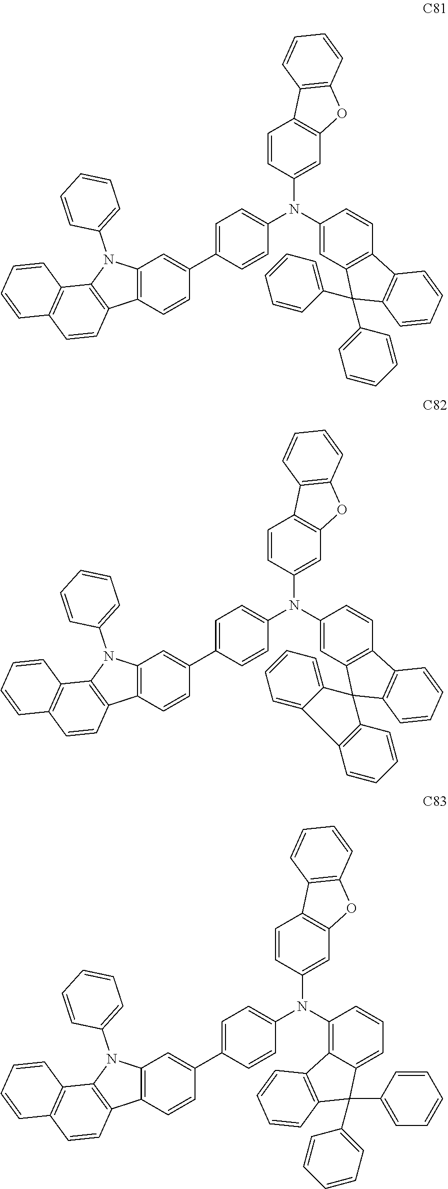 [SMILES: c1ccc(-n2c3cc(-c4ccc(N(c5ccc6c(c5)C(c5ccccc5)(c5ccccc5)c5ccccc5-6)c5ccc6c(c5)oc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6c(c5)oc5ccccc56)cc4)ccc3c3ccc4ccccc4c32)cc1 ; c1ccc(-n2c3cc(-c4ccc(N(c5ccc6c(c5)oc5ccccc56)c5cccc6c5-c5ccccc5C6(c5ccccc5)c5ccccc5)cc4)ccc3c3ccc4ccccc4c32)cc1]

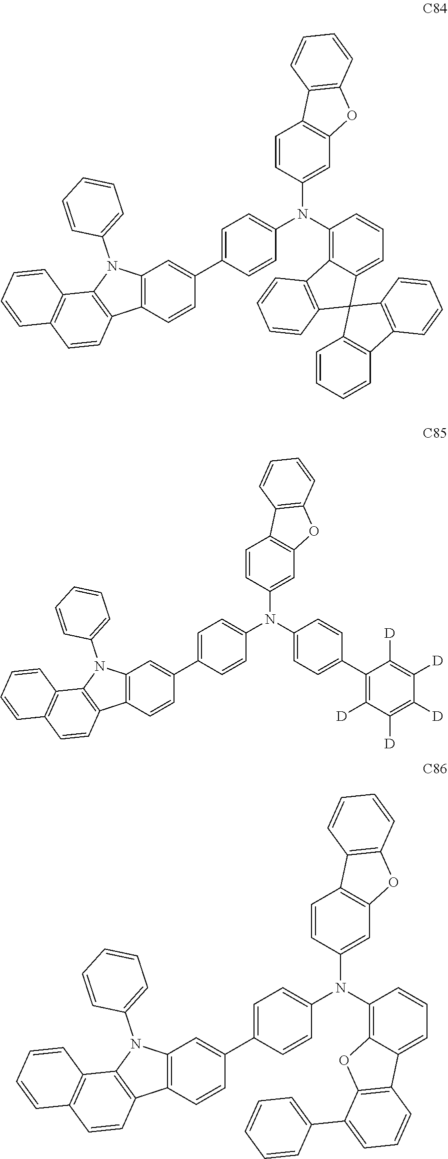 [SMILES: c1ccc(-n2c3cc(-c4ccc(N(c5ccc6c(c5)oc5ccccc56)c5cccc6c5-c5ccccc5C65c6ccccc6-c6ccccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4c(c3)oc3ccccc34)cc2)c([2H])c1[2H] ; c1ccc(-c2cccc3c2oc2c(N(c4ccc(-c5ccc6c7ccc8ccccc8c7n(-c7ccccc7)c6c5)cc4)c4ccc5c(c4)oc4ccccc45)cccc23)cc1]

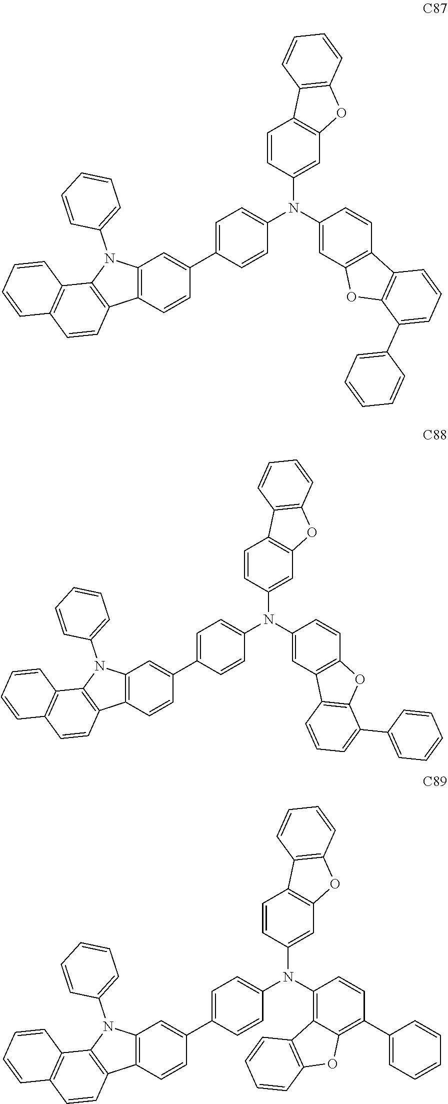 [SMILES: c1ccc(-c2cccc3c2oc2cc(N(c4ccc(-c5ccc6c7ccc8ccccc8c7n(-c7ccccc7)c6c5)cc4)c4ccc5c(c4)oc4ccccc45)ccc23)cc1 ; c1ccc(-c2cccc3c2oc2ccc(N(c4ccc(-c5ccc6c7ccc8ccccc8c7n(-c7ccccc7)c6c5)cc4)c4ccc5c(c4)oc4ccccc45)cc23)cc1 ; c1ccc(-c2ccc(N(c3ccc(-c4ccc5c6ccc7ccccc7c6n(-c6ccccc6)c5c4)cc3)c3ccc4c(c3)oc3ccccc34)c3c2oc2ccccc23)cc1]

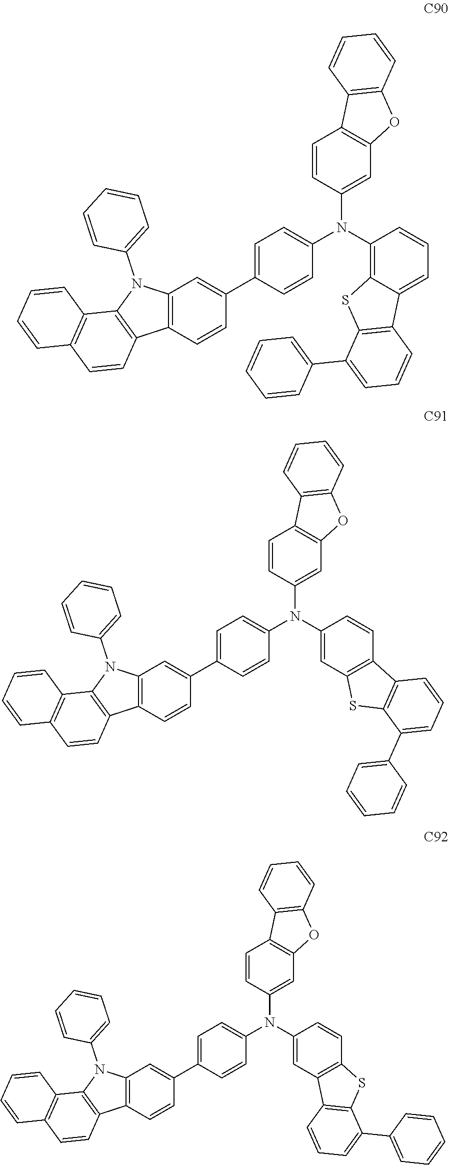 [SMILES: c1ccc(-c2cccc3c2sc2c(N(c4ccc(-c5ccc6c7ccc8ccccc8c7n(-c7ccccc7)c6c5)cc4)c4ccc5c(c4)oc4ccccc45)cccc23)cc1 ; c1ccc(-c2cccc3c2sc2cc(N(c4ccc(-c5ccc6c7ccc8ccccc8c7n(-c7ccccc7)c6c5)cc4)c4ccc5c(c4)oc4ccccc45)ccc23)cc1 ; c1ccc(-c2cccc3c2sc2ccc(N(c4ccc(-c5ccc6c7ccc8ccccc8c7n(-c7ccccc7)c6c5)cc4)c4ccc5c(c4)oc4ccccc45)cc23)cc1]

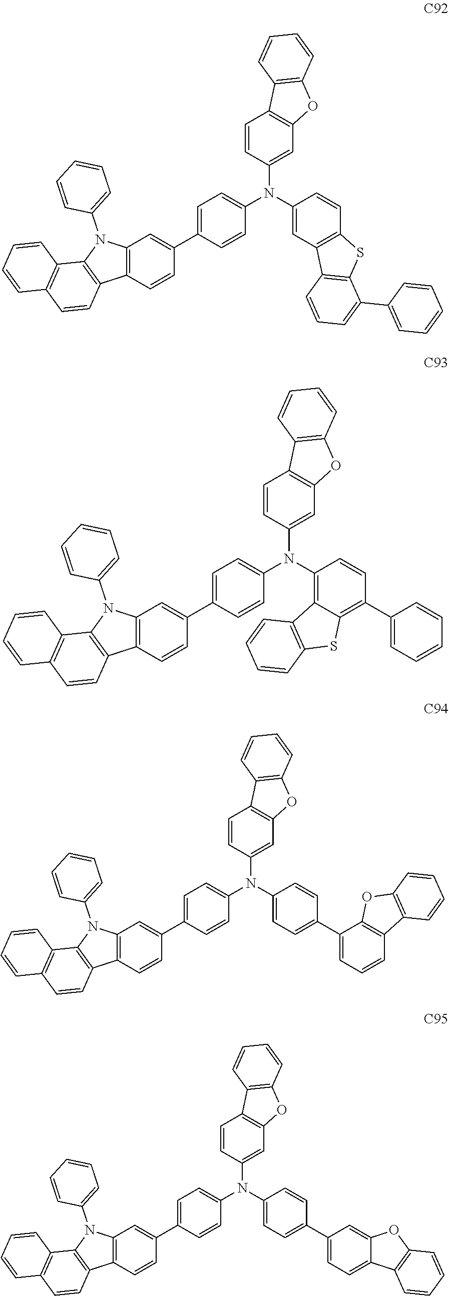 [SMILES: c1ccc(-c2cccc3c2sc2ccc(N(c4ccc(-c5ccc6c7ccc8ccccc8c7n(-c7ccccc7)c6c5)cc4)c4ccc5c(c4)oc4ccccc45)cc23)cc1 ; c1ccc(-c2ccc(N(c3ccc(-c4ccc5c6ccc7ccccc7c6n(-c6ccccc6)c5c4)cc3)c3ccc4c(c3)oc3ccccc34)c3c2sc2ccccc23)cc1 ; c1ccc(-n2c3cc(-c4ccc(N(c5ccc(-c6cccc7c6oc6ccccc67)cc5)c5ccc6c(c5)oc5ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5ccc6c(c5)oc5ccccc56)cc4)ccc3c3ccc4ccccc4c32)cc1]

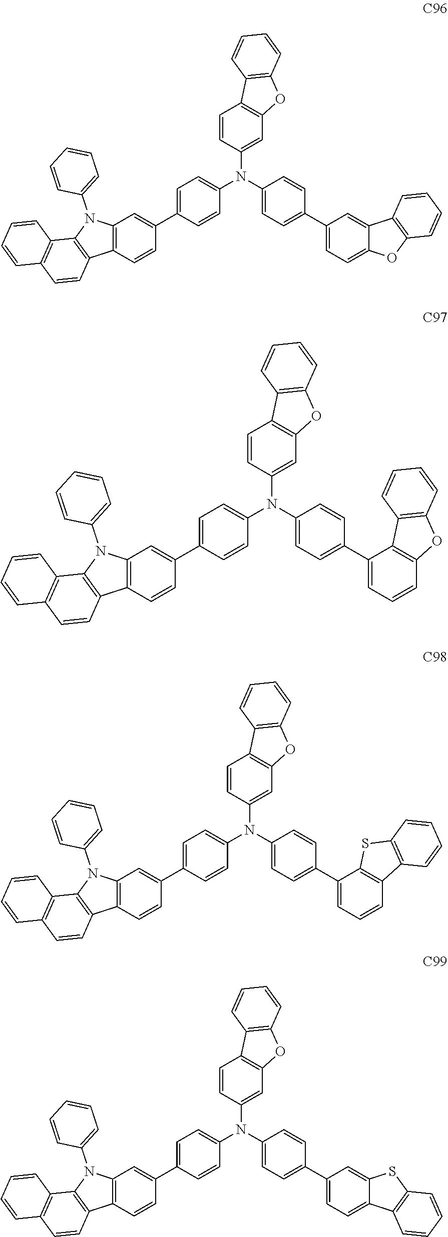 [SMILES: c1ccc(-n2c3cc(-c4ccc(N(c5ccc(-c6ccc7oc8ccccc8c7c6)cc5)c5ccc6c(c5)oc5ccccc56)cc4)ccc3c3ccc4ccccc4c32)cc1 ; c1ccc(-n2c3cc(-c4ccc(N(c5ccc(-c6cccc7oc8ccccc8c67)cc5)c5ccc6c(c5)oc5ccccc56)cc4)ccc3c3ccc4ccccc4c32)cc1 ; c1ccc(-n2c3cc(-c4ccc(N(c5ccc(-c6cccc7c6sc6ccccc67)cc5)c5ccc6c(c5)oc5ccccc56)cc4)ccc3c3ccc4ccccc4c32)cc1 ; c1ccc(-n2c3cc(-c4ccc(N(c5ccc(-c6ccc7c(c6)sc6ccccc67)cc5)c5ccc6c(c5)oc5ccccc56)cc4)ccc3c3ccc4ccccc4c32)cc1]

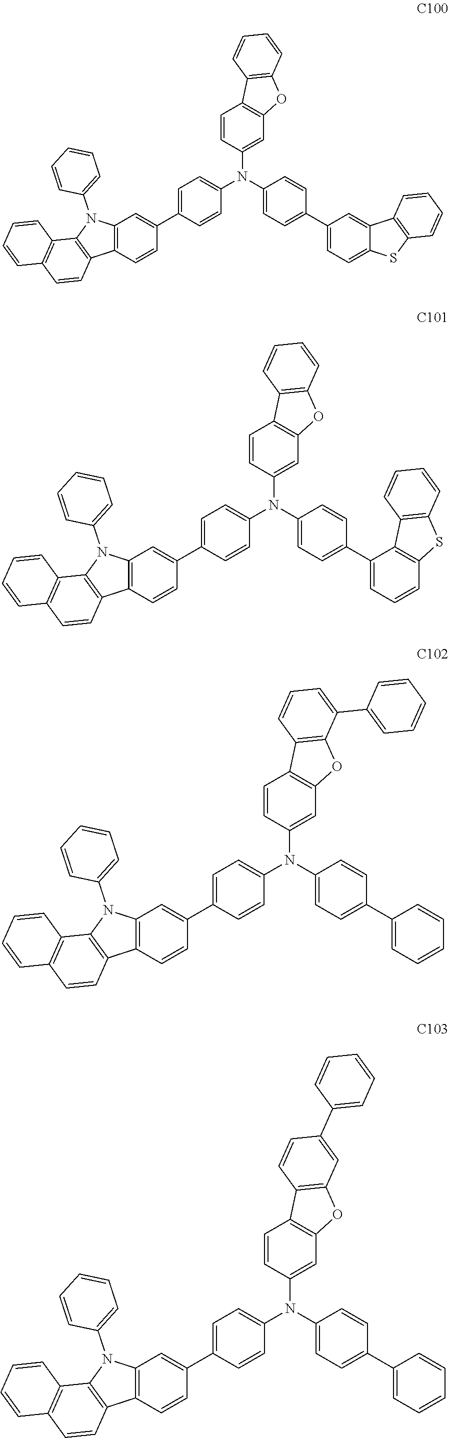 [SMILES: c1ccc(-n2c3cc(-c4ccc(N(c5ccc(-c6ccc7sc8ccccc8c7c6)cc5)c5ccc6c(c5)oc5ccccc56)cc4)ccc3c3ccc4ccccc4c32)cc1 ; c1ccc(-n2c3cc(-c4ccc(N(c5ccc(-c6cccc7sc8ccccc8c67)cc5)c5ccc6c(c5)oc5ccccc56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4c(c3)oc3c(-c5ccccc5)cccc34)cc2)cc1 ; c1ccc(-c2ccc(N(c3ccc(-c4ccc5c6ccc7ccccc7c6n(-c6ccccc6)c5c4)cc3)c3ccc4c(c3)oc3cc(-c5ccccc5)ccc34)cc2)cc1]

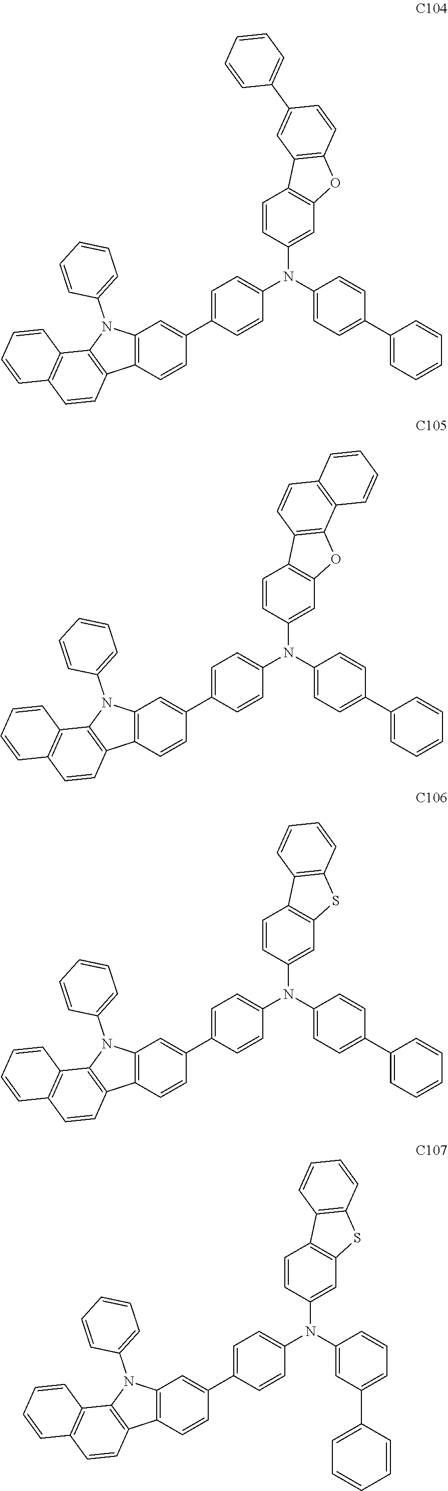 [SMILES: c1ccc(-c2ccc(N(c3ccc(-c4ccc5c6ccc7ccccc7c6n(-c6ccccc6)c5c4)cc3)c3ccc4c(c3)oc3ccc(-c5ccccc5)cc34)cc2)cc1 ; c1ccc(-c2ccc(N(c3ccc(-c4ccc5c6ccc7ccccc7c6n(-c6ccccc6)c5c4)cc3)c3ccc4c(c3)oc3c5ccccc5ccc43)cc2)cc1 ; c1ccc(-c2ccc(N(c3ccc(-c4ccc5c6ccc7ccccc7c6n(-c6ccccc6)c5c4)cc3)c3ccc4c(c3)sc3ccccc34)cc2)cc1 ; c1ccc(-c2cccc(N(c3ccc(-c4ccc5c6ccc7ccccc7c6n(-c6ccccc6)c5c4)cc3)c3ccc4c(c3)sc3ccccc34)c2)cc1]

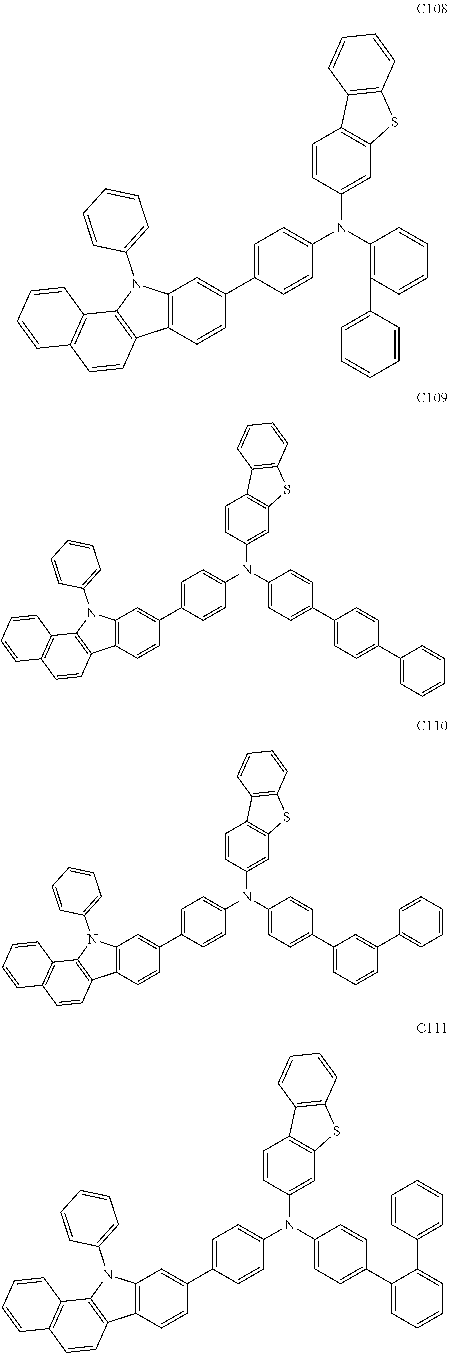 [SMILES: c1ccc(-c2ccccc2N(c2ccc(-c3ccc4c5ccc6ccccc6c5n(-c5ccccc5)c4c3)cc2)c2ccc3c(c2)sc2ccccc23)cc1 ; c1ccc(-c2ccc(-c3ccc(N(c4ccc(-c5ccc6c7ccc8ccccc8c7n(-c7ccccc7)c6c5)cc4)c4ccc5c(c4)sc4ccccc45)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)sc4ccccc45)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(c3)sc3ccccc34)cc2)cc1]

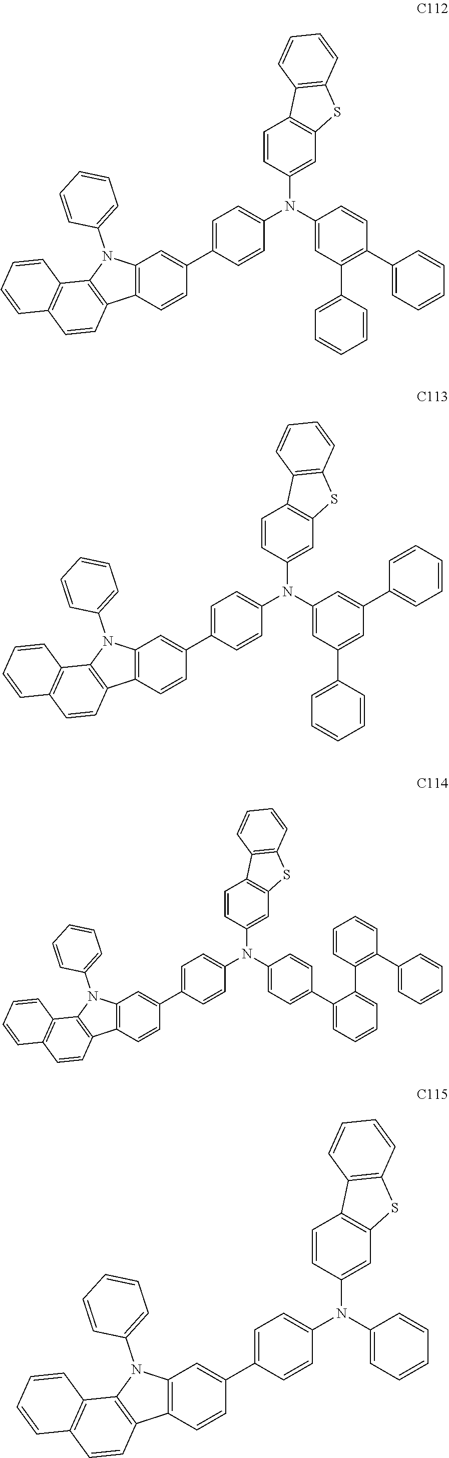 [SMILES: c1ccc(-c2ccc(N(c3ccc(-c4ccc5c6ccc7ccccc7c6n(-c6ccccc6)c5c4)cc3)c3ccc4c(c3)sc3ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(c3)sc3ccccc34)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)sc3ccccc34)cc2)cc1 ; c1ccc(N(c2ccc(-c3ccc4c5ccc6ccccc6c5n(-c5ccccc5)c4c3)cc2)c2ccc3c(c2)sc2ccccc23)cc1]

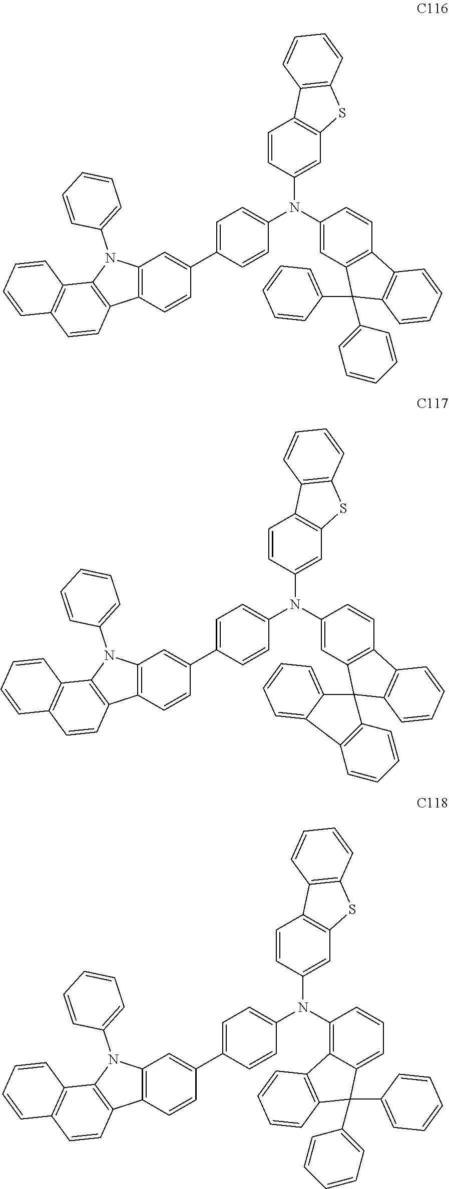 [SMILES: c1ccc(-n2c3cc(-c4ccc(N(c5ccc6c(c5)C(c5ccccc5)(c5ccccc5)c5ccccc5-6)c5ccc6c(c5)sc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6c(c5)sc5ccccc56)cc4)ccc3c3ccc4ccccc4c32)cc1 ; c1ccc(-n2c3cc(-c4ccc(N(c5ccc6c(c5)sc5ccccc56)c5cccc6c5-c5ccccc5C6(c5ccccc5)c5ccccc5)cc4)ccc3c3ccc4ccccc4c32)cc1]

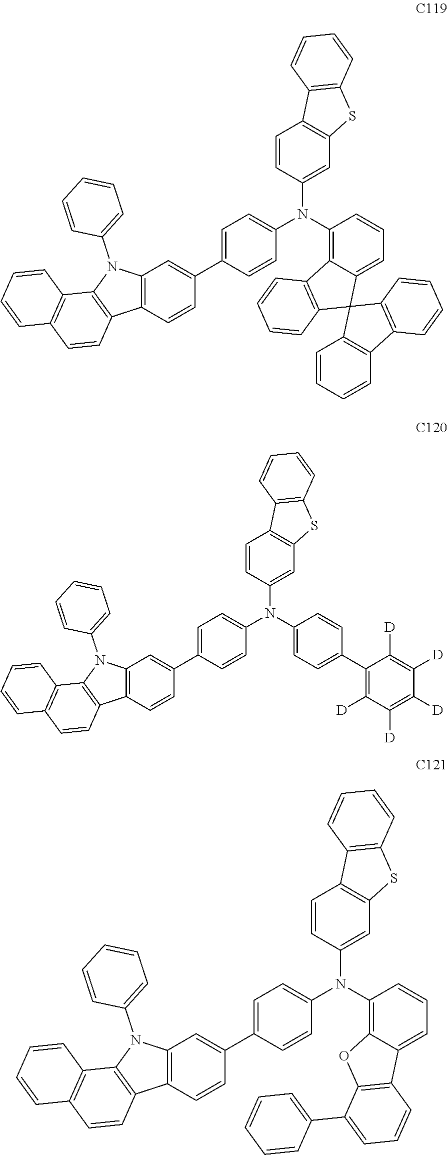 [SMILES: c1ccc(-n2c3cc(-c4ccc(N(c5ccc6c(c5)sc5ccccc56)c5cccc6c5-c5ccccc5C65c6ccccc6-c6ccccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4c(c3)sc3ccccc34)cc2)c([2H])c1[2H] ; c1ccc(-c2cccc3c2oc2c(N(c4ccc(-c5ccc6c7ccc8ccccc8c7n(-c7ccccc7)c6c5)cc4)c4ccc5c(c4)sc4ccccc45)cccc23)cc1]

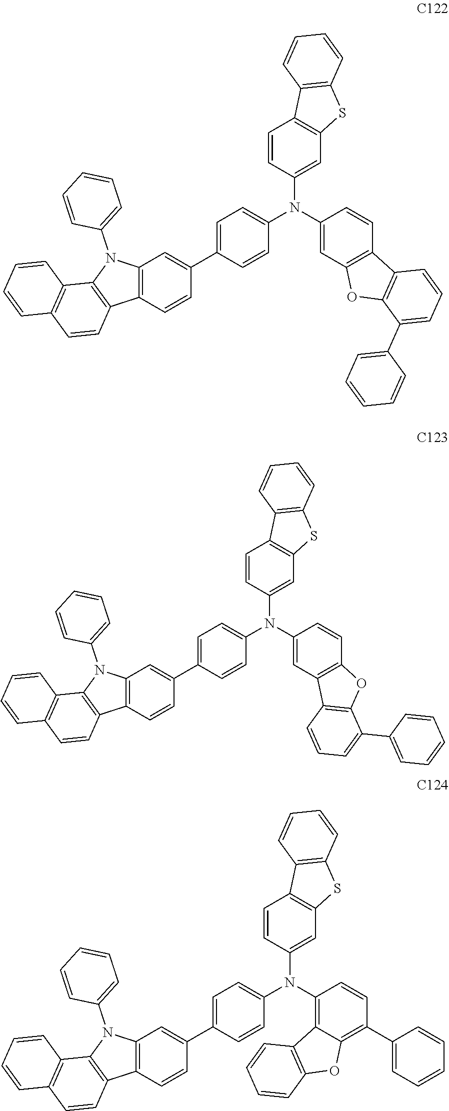 [SMILES: c1ccc(-c2cccc3c2oc2cc(N(c4ccc(-c5ccc6c7ccc8ccccc8c7n(-c7ccccc7)c6c5)cc4)c4ccc5c(c4)sc4ccccc45)ccc23)cc1 ; c1ccc(-c2cccc3c2oc2ccc(N(c4ccc(-c5ccc6c7ccc8ccccc8c7n(-c7ccccc7)c6c5)cc4)c4ccc5c(c4)sc4ccccc45)cc23)cc1 ; c1ccc(-c2ccc(N(c3ccc(-c4ccc5c6ccc7ccccc7c6n(-c6ccccc6)c5c4)cc3)c3ccc4c(c3)sc3ccccc34)c3c2oc2ccccc23)cc1]

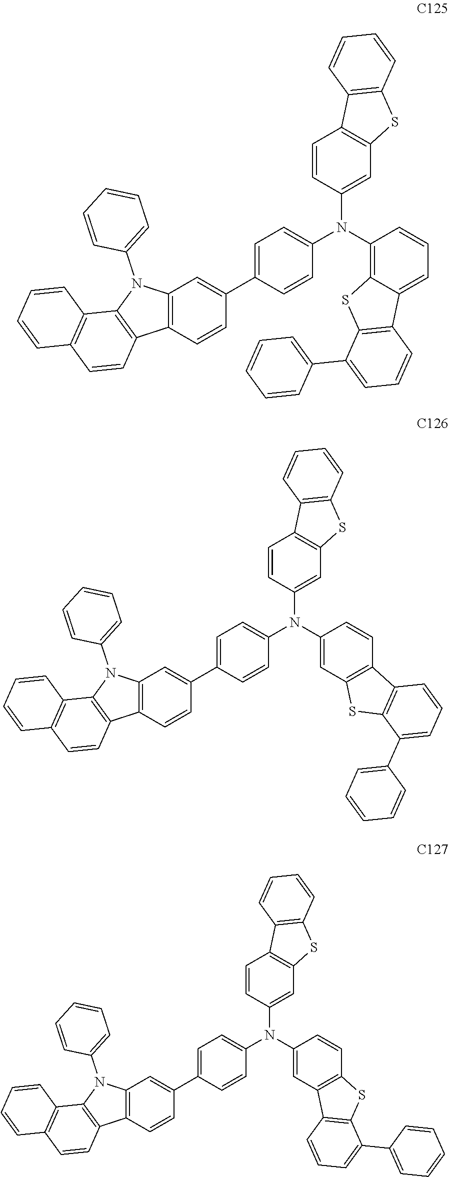 [SMILES: c1ccc(-c2cccc3c2sc2c(N(c4ccc(-c5ccc6c7ccc8ccccc8c7n(-c7ccccc7)c6c5)cc4)c4ccc5c(c4)sc4ccccc45)cccc23)cc1 ; c1ccc(-c2cccc3c2sc2cc(N(c4ccc(-c5ccc6c7ccc8ccccc8c7n(-c7ccccc7)c6c5)cc4)c4ccc5c(c4)sc4ccccc45)ccc23)cc1 ; c1ccc(-c2cccc3c2sc2ccc(N(c4ccc(-c5ccc6c7ccc8ccccc8c7n(-c7ccccc7)c6c5)cc4)c4ccc5c(c4)sc4ccccc45)cc23)cc1]

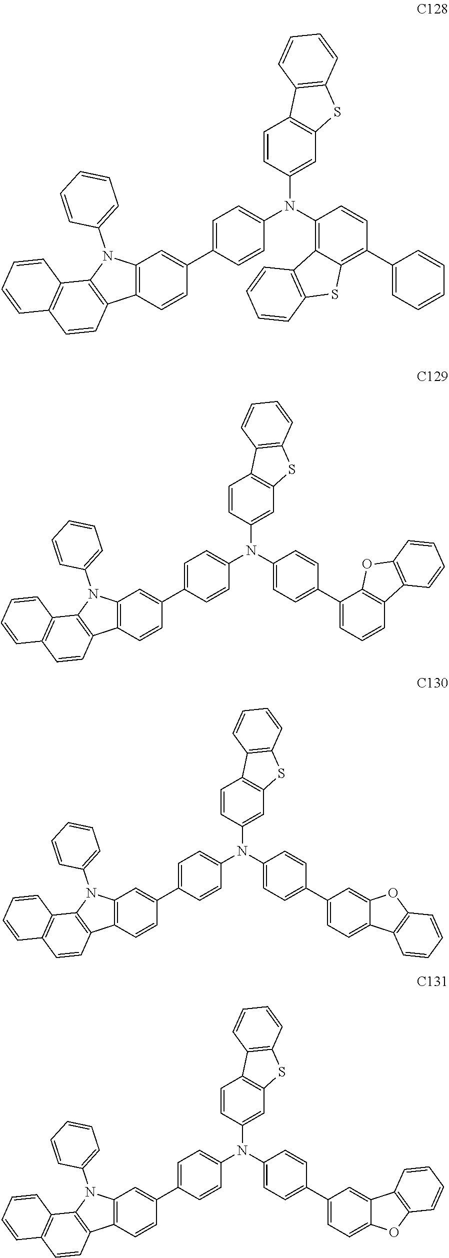 [SMILES: c1ccc(-c2ccc(N(c3ccc(-c4ccc5c6ccc7ccccc7c6n(-c6ccccc6)c5c4)cc3)c3ccc4c(c3)sc3ccccc34)c3c2sc2ccccc23)cc1 ; c1ccc(-n2c3cc(-c4ccc(N(c5ccc(-c6cccc7c6oc6ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6ccc7oc8ccccc8c7c6)cc5)c5ccc6c(c5)sc5ccccc56)cc4)ccc3c3ccc4ccccc4c32)cc1]

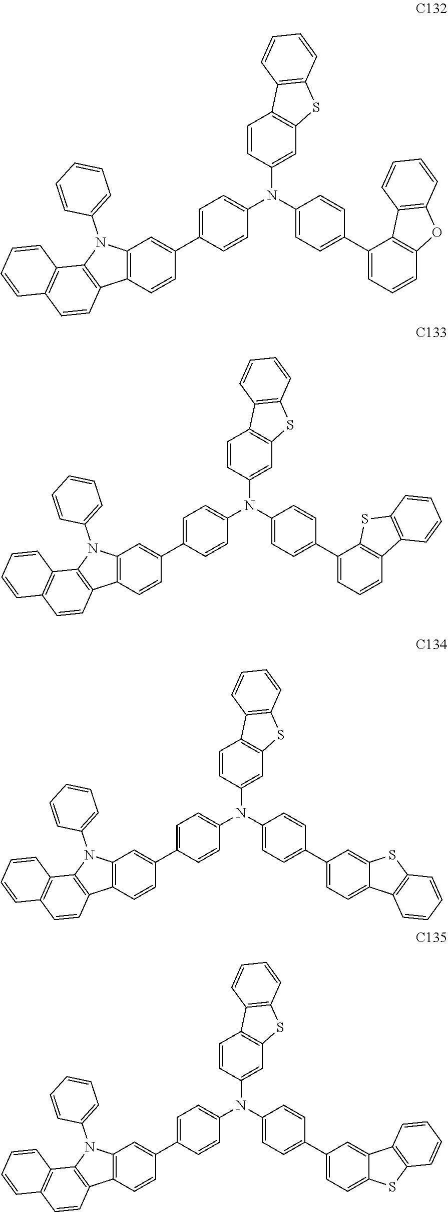 [SMILES: c1ccc(-n2c3cc(-c4ccc(N(c5ccc(-c6cccc7oc8ccccc8c67)cc5)c5ccc6c(c5)sc5ccccc56)cc4)ccc3c3ccc4ccccc4c32)cc1 ; c1ccc(-n2c3cc(-c4ccc(N(c5ccc(-c6cccc7c6sc6ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6ccc7c(c6)sc6ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6ccc7sc8ccccc8c7c6)cc5)c5ccc6c(c5)sc5ccccc56)cc4)ccc3c3ccc4ccccc4c32)cc1]

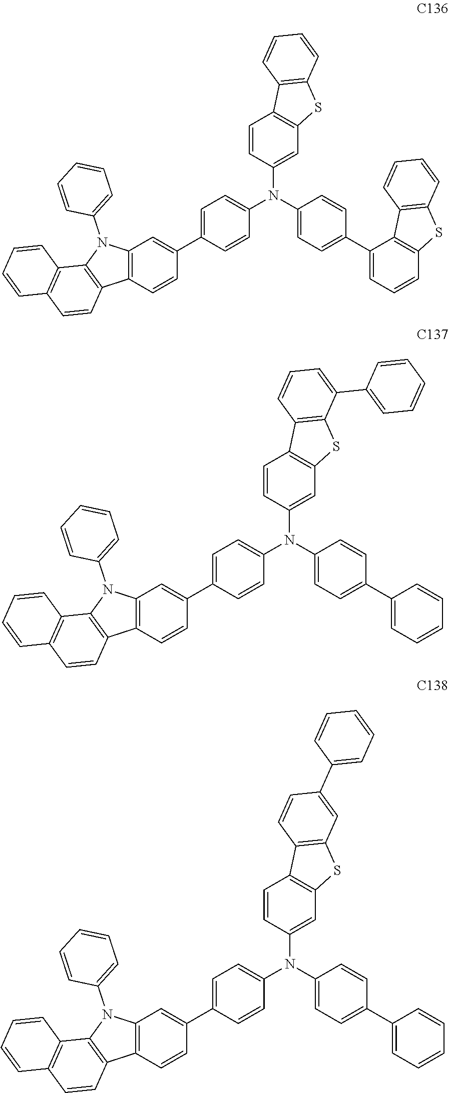 [SMILES: c1ccc(-n2c3cc(-c4ccc(N(c5ccc(-c6cccc7sc8ccccc8c67)cc5)c5ccc6c(c5)sc5ccccc56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4c(c3)sc3c(-c5ccccc5)cccc34)cc2)cc1 ; c1ccc(-c2ccc(N(c3ccc(-c4ccc5c6ccc7ccccc7c6n(-c6ccccc6)c5c4)cc3)c3ccc4c(c3)sc3cc(-c5ccccc5)ccc34)cc2)cc1]

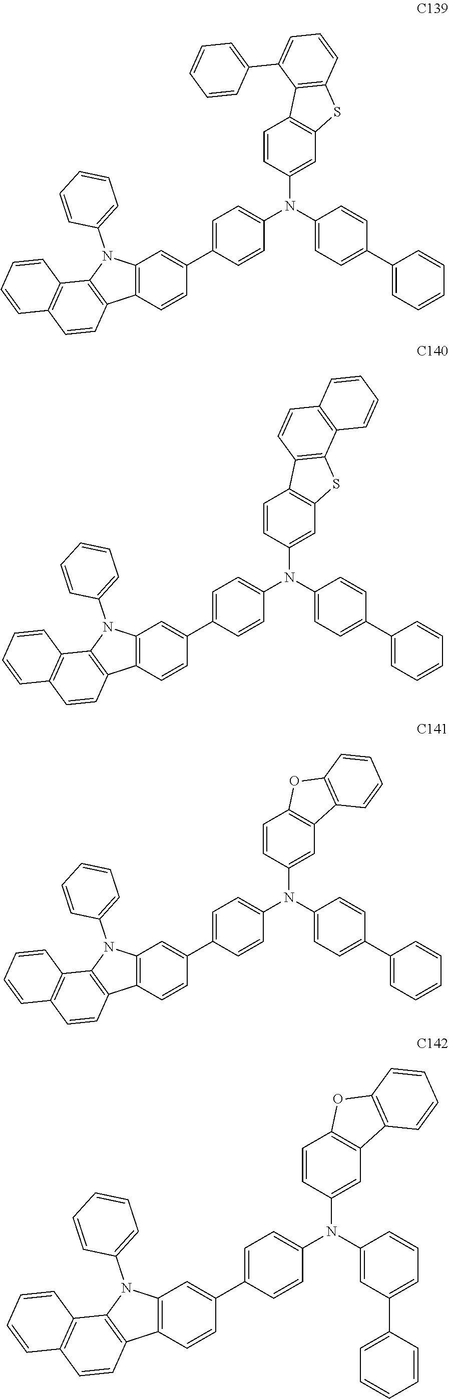 [SMILES: c1ccc(-c2ccc(N(c3ccc(-c4ccc5c6ccc7ccccc7c6n(-c6ccccc6)c5c4)cc3)c3ccc4c(c3)sc3cccc(-c5ccccc5)c34)cc2)cc1 ; c1ccc(-c2ccc(N(c3ccc(-c4ccc5c6ccc7ccccc7c6n(-c6ccccc6)c5c4)cc3)c3ccc4c(c3)sc3c5ccccc5ccc43)cc2)cc1 ; c1ccc(-c2ccc(N(c3ccc(-c4ccc5c6ccc7ccccc7c6n(-c6ccccc6)c5c4)cc3)c3ccc4oc5ccccc5c4c3)cc2)cc1 ; c1ccc(-c2cccc(N(c3ccc(-c4ccc5c6ccc7ccccc7c6n(-c6ccccc6)c5c4)cc3)c3ccc4oc5ccccc5c4c3)c2)cc1]

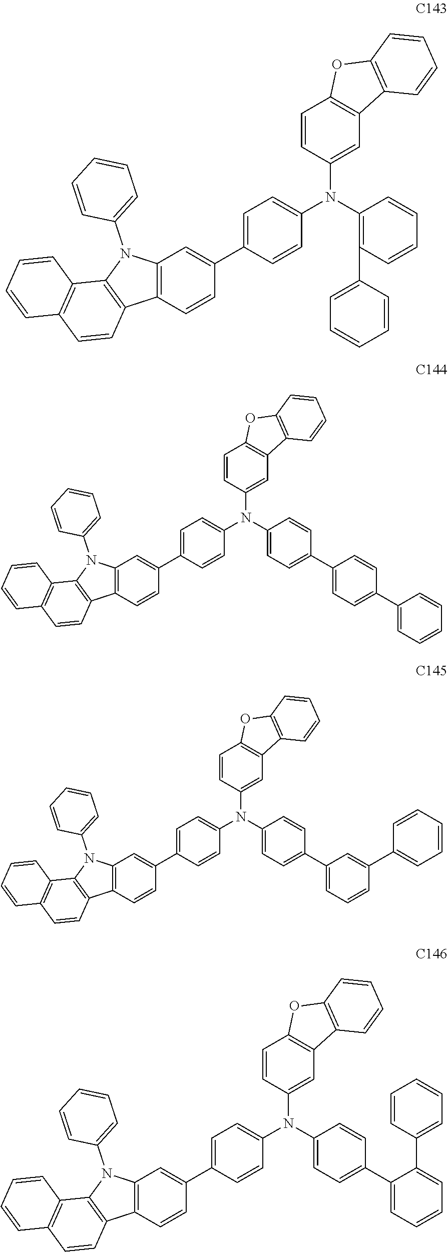 [SMILES: c1ccc(-c2ccccc2N(c2ccc(-c3ccc4c5ccc6ccccc6c5n(-c5ccccc5)c4c3)cc2)c2ccc3oc4ccccc4c3c2)cc1 ; c1ccc(-c2ccc(-c3ccc(N(c4ccc(-c5ccc6c7ccc8ccccc8c7n(-c7ccccc7)c6c5)cc4)c4ccc5oc6ccccc6c5c4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5oc6ccccc6c5c4)cc3)c2)cc1 ; c1ccc(-c2ccccc2-c2ccc(N(c3ccc(-c4ccc5c6ccc7ccccc7c6n(-c6ccccc6)c5c4)cc3)c3ccc4oc5ccccc5c4c3)cc2)cc1]

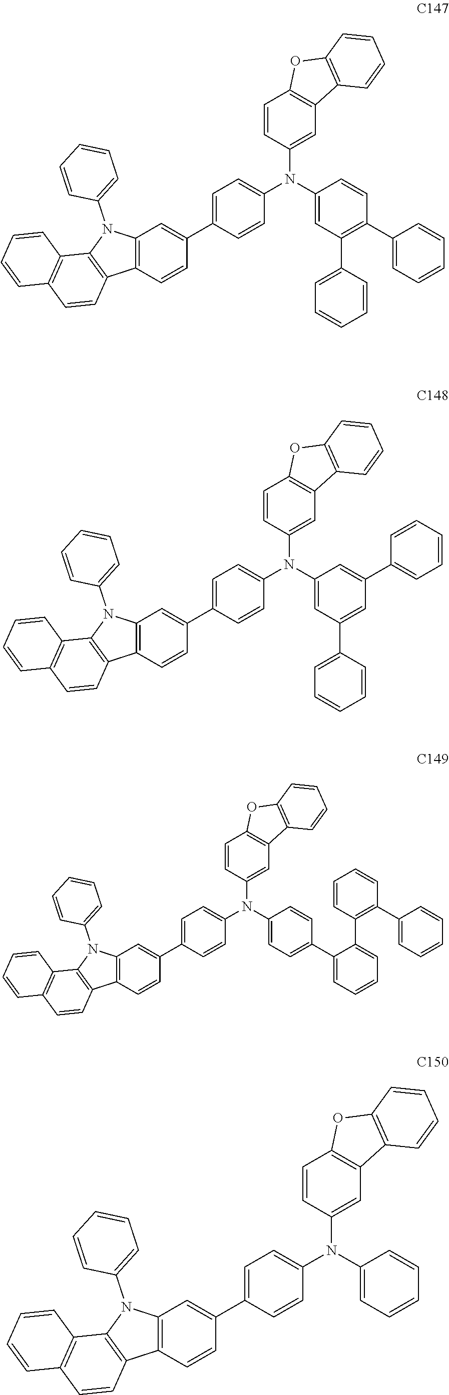 [SMILES: c1ccc(-c2ccc(N(c3ccc(-c4ccc5c6ccc7ccccc7c6n(-c6ccccc6)c5c4)cc3)c3ccc4oc5ccccc5c4c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4oc5ccccc5c4c3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4oc5ccccc5c4c3)cc2)cc1 ; c1ccc(N(c2ccc(-c3ccc4c5ccc6ccccc6c5n(-c5ccccc5)c4c3)cc2)c2ccc3oc4ccccc4c3c2)cc1]

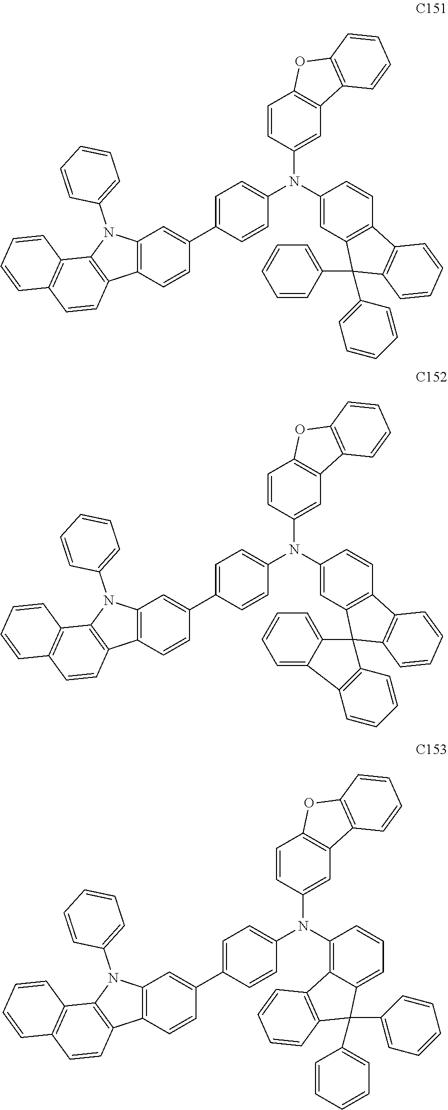 [SMILES: c1ccc(-n2c3cc(-c4ccc(N(c5ccc6c(c5)C(c5ccccc5)(c5ccccc5)c5ccccc5-6)c5ccc6oc7ccccc7c6c5)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6oc7ccccc7c6c5)cc4)ccc3c3ccc4ccccc4c32)cc1 ; c1ccc(-n2c3cc(-c4ccc(N(c5ccc6oc7ccccc7c6c5)c5cccc6c5-c5ccccc5C6(c5ccccc5)c5ccccc5)cc4)ccc3c3ccc4ccccc4c32)cc1]

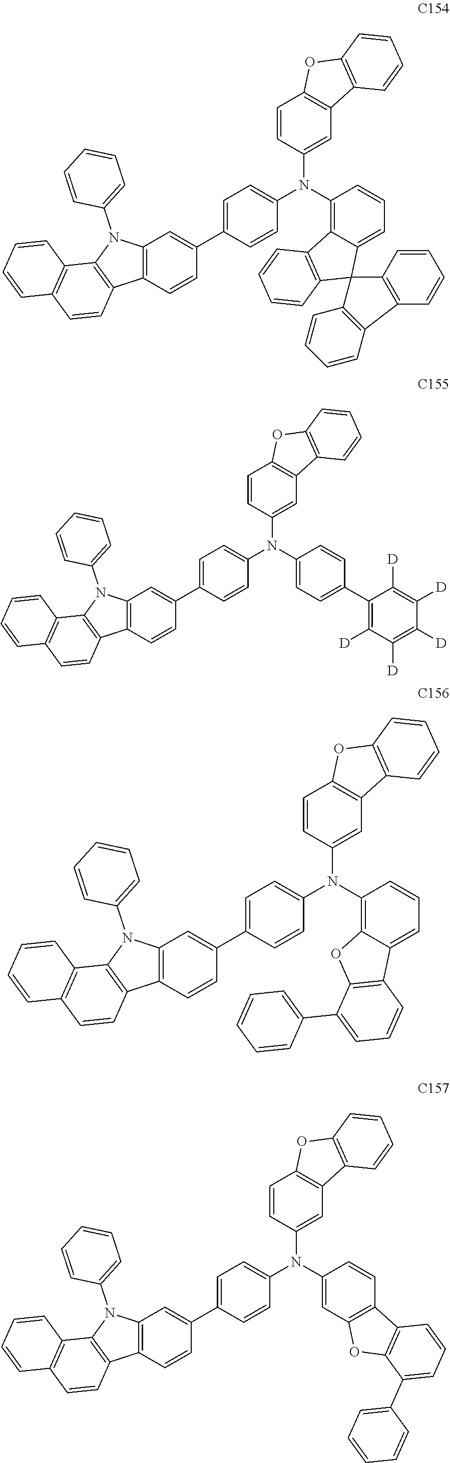

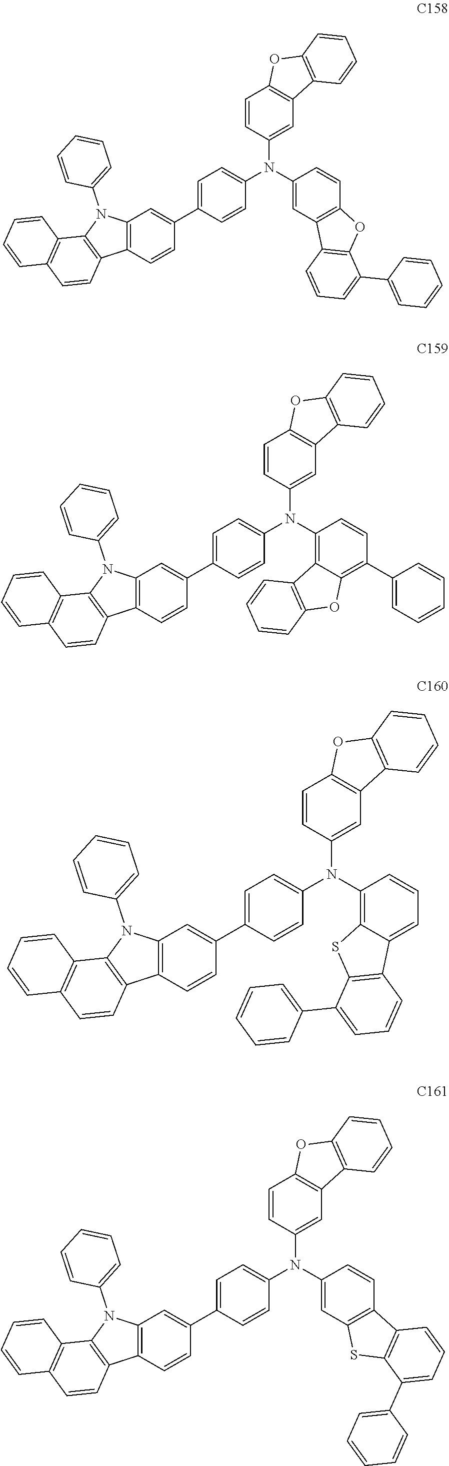 [SMILES: c1ccc(-c2cccc3c2oc2ccc(N(c4ccc(-c5ccc6c7ccc8ccccc8c7n(-c7ccccc7)c6c5)cc4)c4ccc5oc6ccccc6c5c4)cc23)cc1 ; c1ccc(-c2ccc(N(c3ccc(-c4ccc5c6ccc7ccccc7c6n(-c6ccccc6)c5c4)cc3)c3ccc4oc5ccccc5c4c3)c3c2oc2ccccc23)cc1 ; c1ccc(-c2cccc3c2sc2c(N(c4ccc(-c5ccc6c7ccc8ccccc8c7n(-c7ccccc7)c6c5)cc4)c4ccc5oc6ccccc6c5c4)cccc23)cc1 ; c1ccc(-c2cccc3c2sc2cc(N(c4ccc(-c5ccc6c7ccc8ccccc8c7n(-c7ccccc7)c6c5)cc4)c4ccc5oc6ccccc6c5c4)ccc23)cc1]

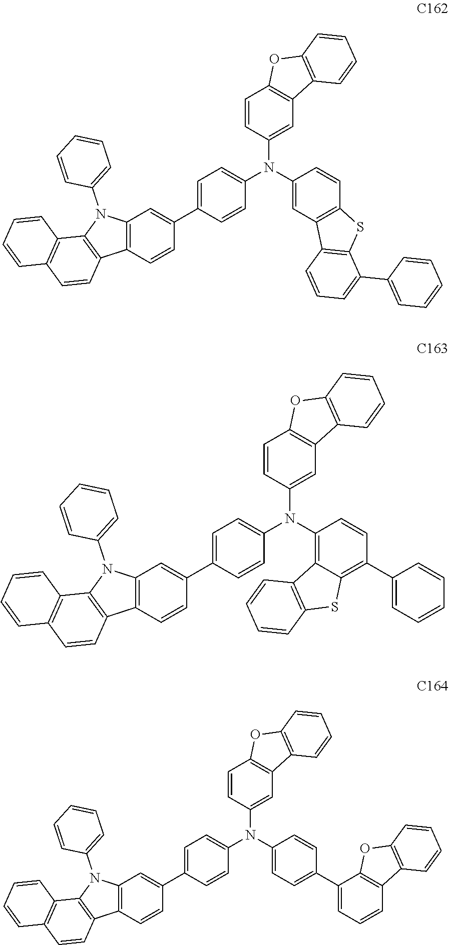 [SMILES: c1ccc(-c2cccc3c2sc2ccc(N(c4ccc(-c5ccc6c7ccc8ccccc8c7n(-c7ccccc7)c6c5)cc4)c4ccc5oc6ccccc6c5c4)cc23)cc1 ; c1ccc(-c2ccc(N(c3ccc(-c4ccc5c6ccc7ccccc7c6n(-c6ccccc6)c5c4)cc3)c3ccc4oc5ccccc5c4c3)c3c2sc2ccccc23)cc1 ; c1ccc(-n2c3cc(-c4ccc(N(c5ccc(-c6cccc7c6oc6ccccc67)cc5)c5ccc6oc7ccccc7c6c5)cc4)ccc3c3ccc4ccccc4c32)cc1]

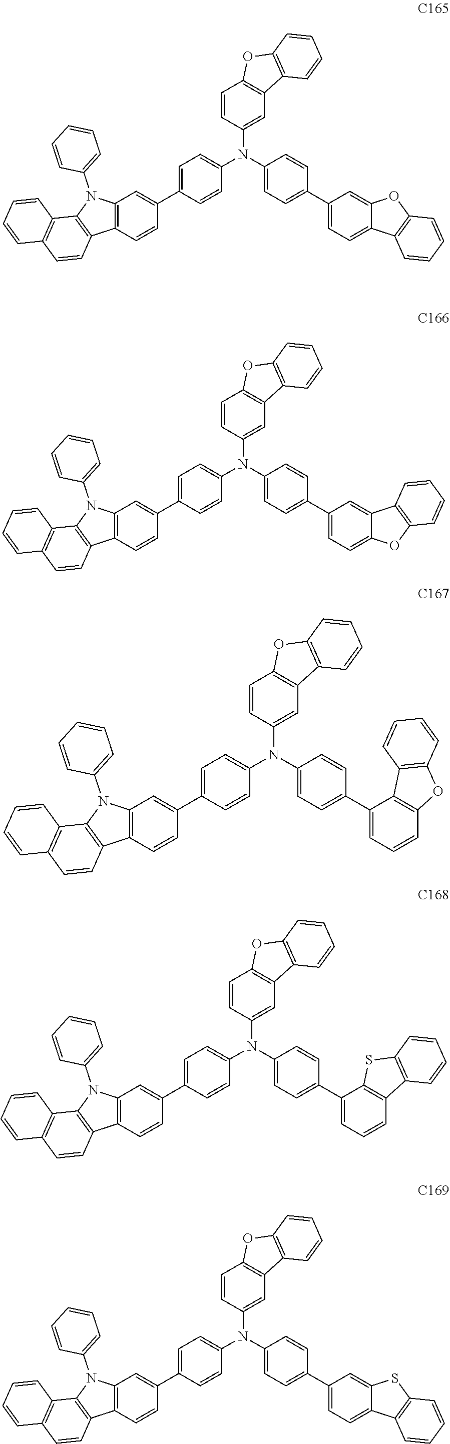

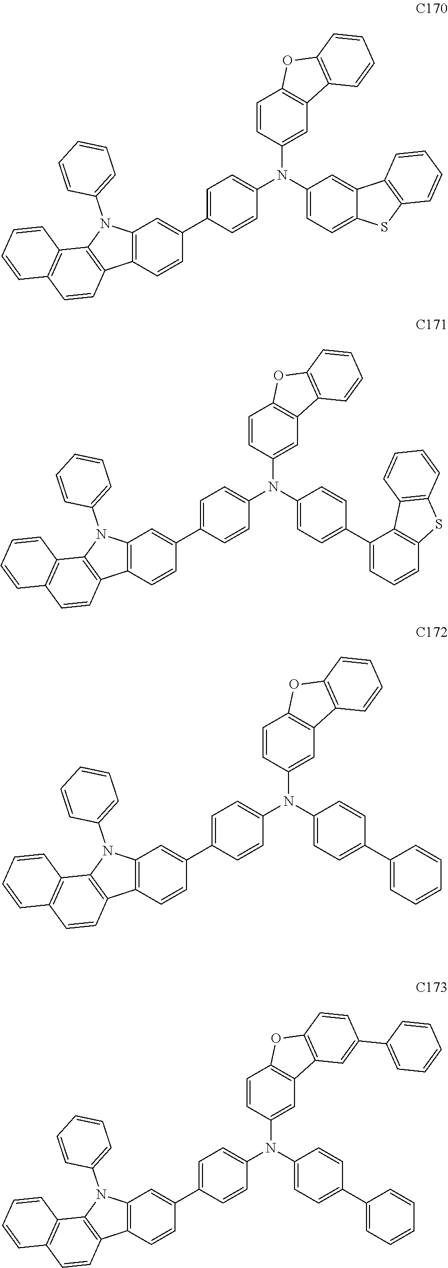 [SMILES: c1ccc(-n2c3cc(-c4ccc(N(c5ccc6oc7ccccc7c6c5)c5ccc6sc7ccccc7c6c5)cc4)ccc3c3ccc4ccccc4c32)cc1 ; c1ccc(-n2c3cc(-c4ccc(N(c5ccc(-c6cccc7sc8ccccc8c67)cc5)c5ccc6oc7ccccc7c6c5)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4oc5ccccc5c4c3)cc2)cc1 ; c1ccc(-c2ccc(N(c3ccc(-c4ccc5c6ccc7ccccc7c6n(-c6ccccc6)c5c4)cc3)c3ccc4oc5ccc(-c6ccccc6)cc5c4c3)cc2)cc1]

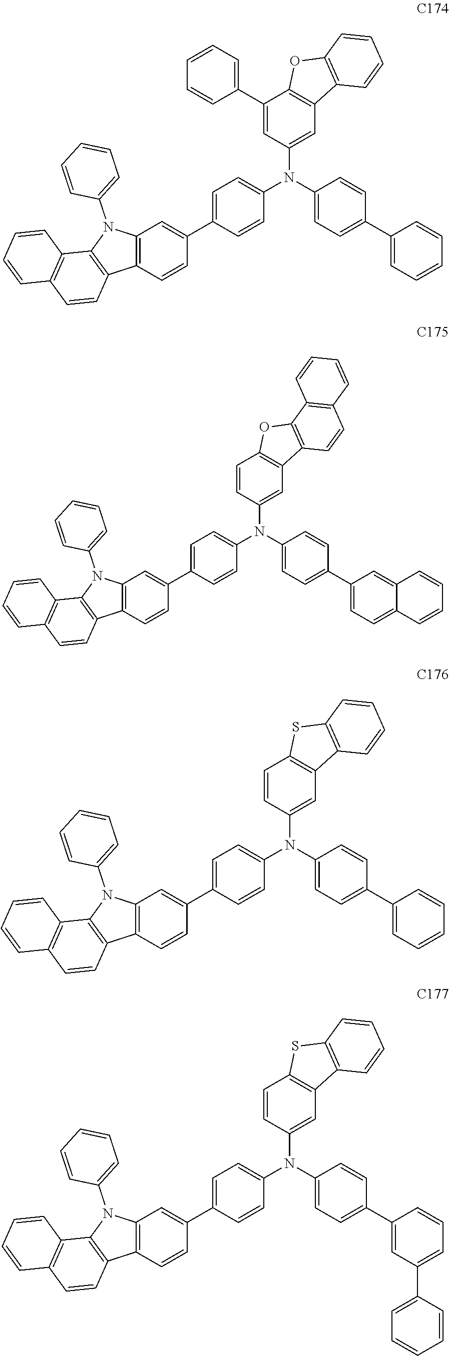 [SMILES: c1ccc(-c2ccc(N(c3ccc(-c4ccc5c6ccc7ccccc7c6n(-c6ccccc6)c5c4)cc3)c3cc(-c4ccccc4)c4oc5ccccc5c4c3)cc2)cc1 ; c1ccc(-n2c3cc(-c4ccc(N(c5ccc(-c6ccc7ccccc7c6)cc5)c5ccc6oc7c8ccccc8ccc7c6c5)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4sc5ccccc5c4c3)cc2)cc1 ; c1ccc(-c2cccc(-c3ccc(N(c4ccc(-c5ccc6c7ccc8ccccc8c7n(-c7ccccc7)c6c5)cc4)c4ccc5sc6ccccc6c5c4)cc3)c2)cc1]

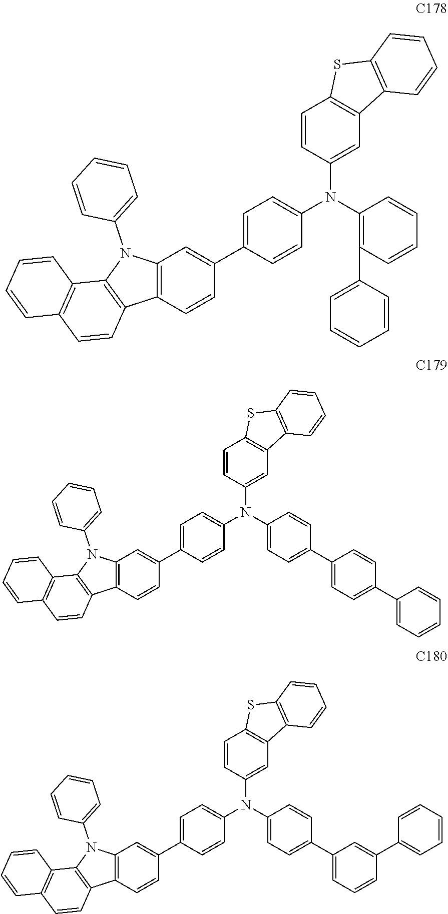 [SMILES: c1ccc(-c2ccccc2N(c2ccc(-c3ccc4c5ccc6ccccc6c5n(-c5ccccc5)c4c3)cc2)c2ccc3sc4ccccc4c3c2)cc1 ; c1ccc(-c2ccc(-c3ccc(N(c4ccc(-c5ccc6c7ccc8ccccc8c7n(-c7ccccc7)c6c5)cc4)c4ccc5sc6ccccc6c5c4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5sc6ccccc6c5c4)cc3)c2)cc1]

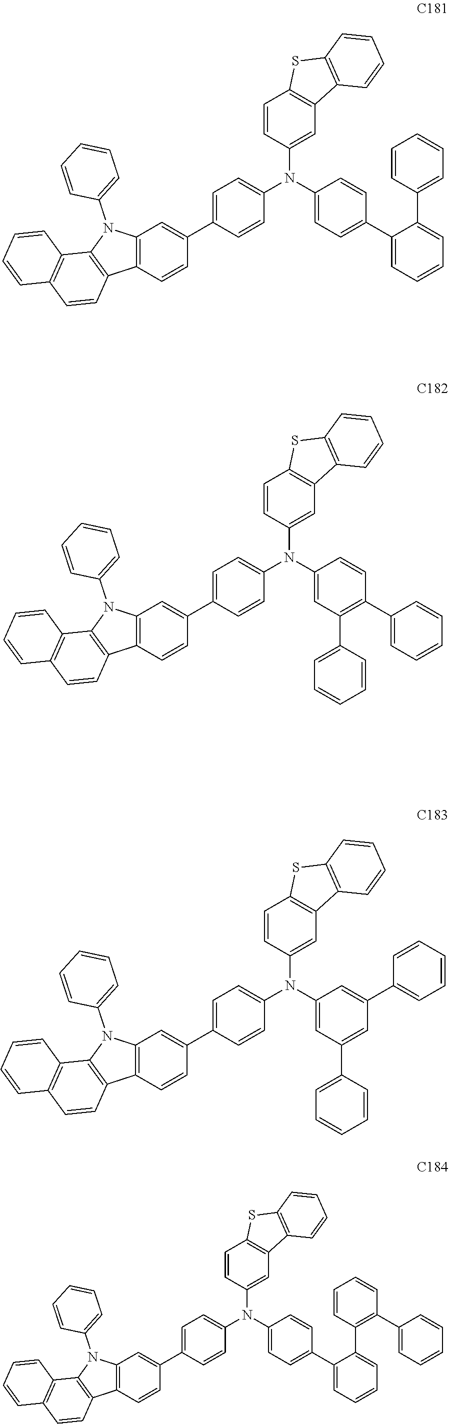 [SMILES: c1ccc(-c2ccccc2-c2ccc(N(c3ccc(-c4ccc5c6ccc7ccccc7c6n(-c6ccccc6)c5c4)cc3)c3ccc4sc5ccccc5c4c3)cc2)cc1 ; c1ccc(-c2ccc(N(c3ccc(-c4ccc5c6ccc7ccccc7c6n(-c6ccccc6)c5c4)cc3)c3ccc4sc5ccccc5c4c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4sc5ccccc5c4c3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4sc5ccccc5c4c3)cc2)cc1]

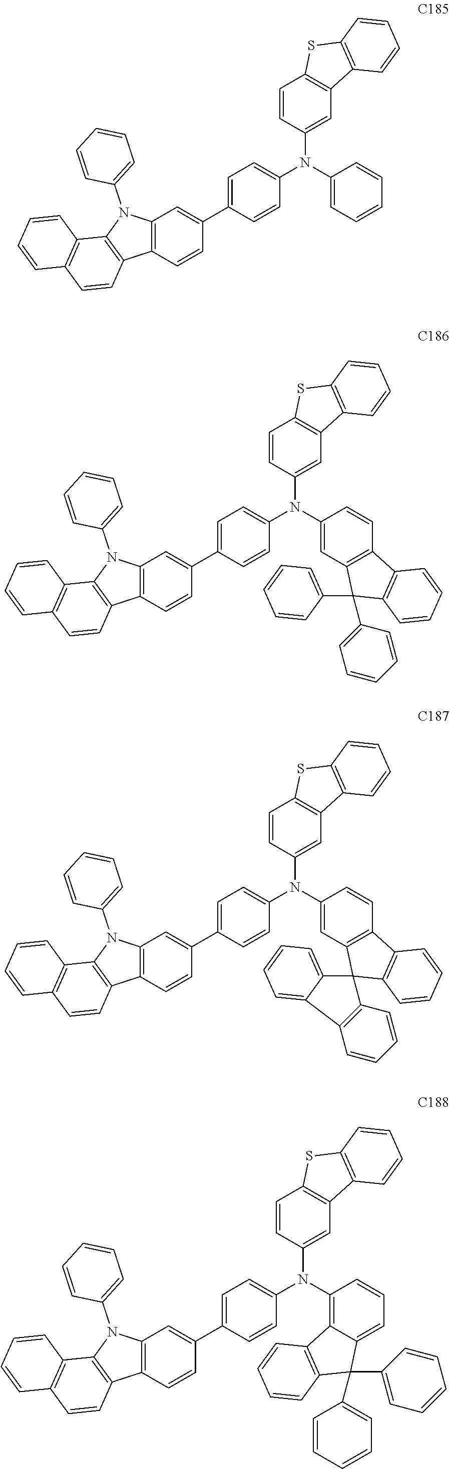 [SMILES: c1ccc(N(c2ccc(-c3ccc4c5ccc6ccccc6c5n(-c5ccccc5)c4c3)cc2)c2ccc3sc4ccccc4c3c2)cc1 ; c1ccc(-n2c3cc(-c4ccc(N(c5ccc6c(c5)C(c5ccccc5)(c5ccccc5)c5ccccc5-6)c5ccc6sc7ccccc7c6c5)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6sc7ccccc7c6c5)cc4)ccc3c3ccc4ccccc4c32)cc1 ; c1ccc(-n2c3cc(-c4ccc(N(c5ccc6sc7ccccc7c6c5)c5cccc6c5-c5ccccc5C6(c5ccccc5)c5ccccc5)cc4)ccc3c3ccc4ccccc4c32)cc1]

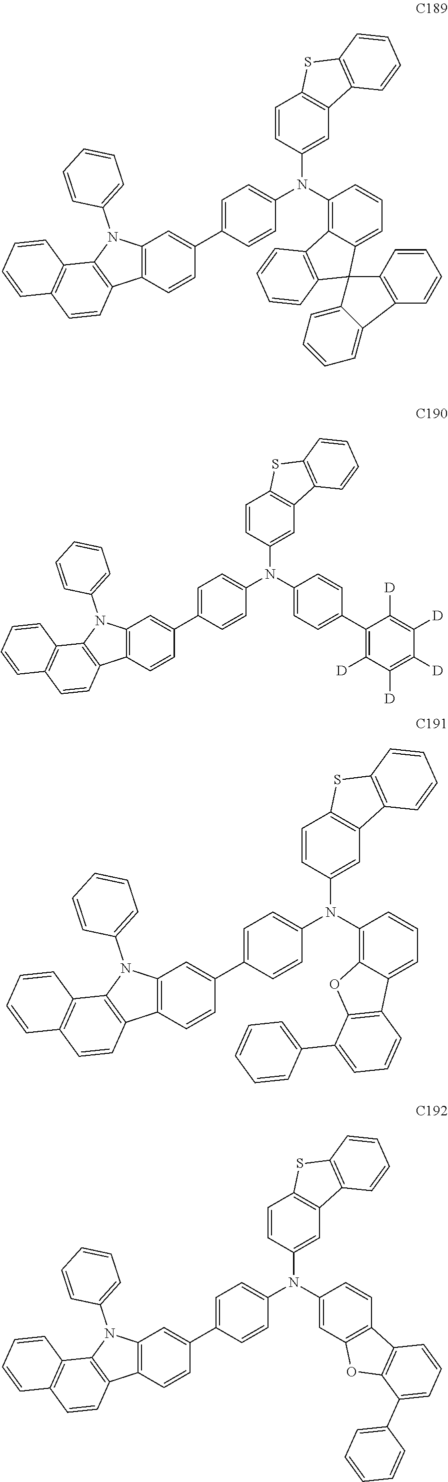 [SMILES: c1ccc(-n2c3cc(-c4ccc(N(c5ccc6sc7ccccc7c6c5)c5cccc6c5-c5ccccc5C65c6ccccc6-c6ccccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4sc5ccccc5c4c3)cc2)c([2H])c1[2H] ; c1ccc(-c2cccc3c2oc2c(N(c4ccc(-c5ccc6c7ccc8ccccc8c7n(-c7ccccc7)c6c5)cc4)c4ccc5sc6ccccc6c5c4)cccc23)cc1 ; c1ccc(-c2cccc3c2oc2cc(N(c4ccc(-c5ccc6c7ccc8ccccc8c7n(-c7ccccc7)c6c5)cc4)c4ccc5sc6ccccc6c5c4)ccc23)cc1]

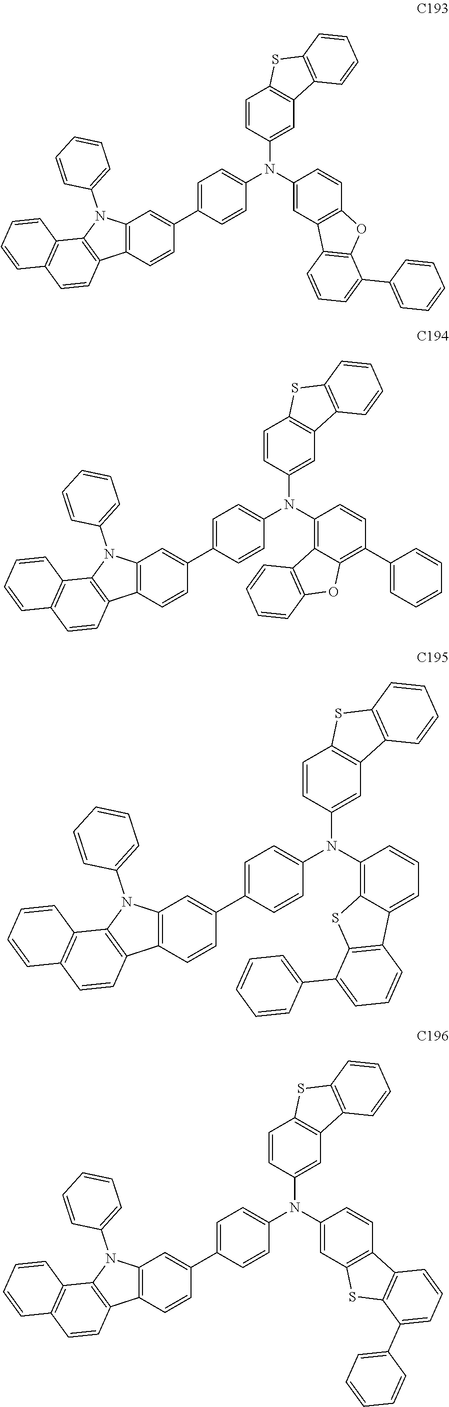 [SMILES: c1ccc(-c2cccc3c2oc2ccc(N(c4ccc(-c5ccc6c7ccc8ccccc8c7n(-c7ccccc7)c6c5)cc4)c4ccc5sc6ccccc6c5c4)cc23)cc1 ; c1ccc(-c2ccc(N(c3ccc(-c4ccc5c6ccc7ccccc7c6n(-c6ccccc6)c5c4)cc3)c3ccc4sc5ccccc5c4c3)c3c2oc2ccccc23)cc1 ; c1ccc(-c2cccc3c2sc2c(N(c4ccc(-c5ccc6c7ccc8ccccc8c7n(-c7ccccc7)c6c5)cc4)c4ccc5sc6ccccc6c5c4)cccc23)cc1 ; c1ccc(-c2cccc3c2sc2cc(N(c4ccc(-c5ccc6c7ccc8ccccc8c7n(-c7ccccc7)c6c5)cc4)c4ccc5sc6ccccc6c5c4)ccc23)cc1]

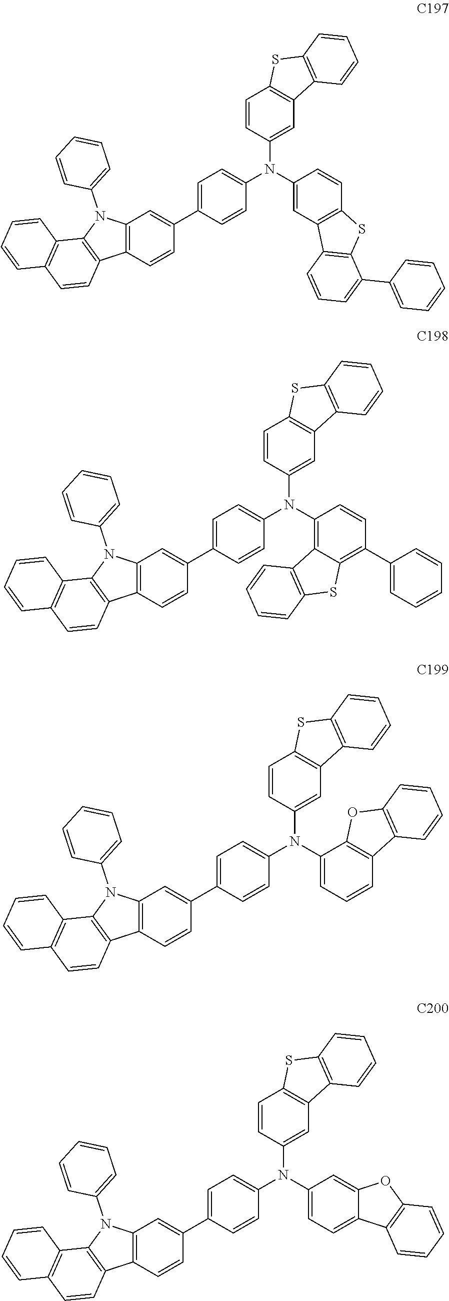 [SMILES: c1ccc(-c2cccc3c2sc2ccc(N(c4ccc(-c5ccc6c7ccc8ccccc8c7n(-c7ccccc7)c6c5)cc4)c4ccc5sc6ccccc6c5c4)cc23)cc1 ; c1ccc(-c2ccc(N(c3ccc(-c4ccc5c6ccc7ccccc7c6n(-c6ccccc6)c5c4)cc3)c3ccc4sc5ccccc5c4c3)c3c2sc2ccccc23)cc1 ; c1ccc(-n2c3cc(-c4ccc(N(c5ccc6sc7ccccc7c6c5)c5cccc6c5oc5ccccc56)cc4)ccc3c3ccc4ccccc4c32)cc1 ; c1ccc(-n2c3cc(-c4ccc(N(c5ccc6c(c5)oc5ccccc56)c5ccc6sc7ccccc7c6c5)cc4)ccc3c3ccc4ccccc4c32)cc1]

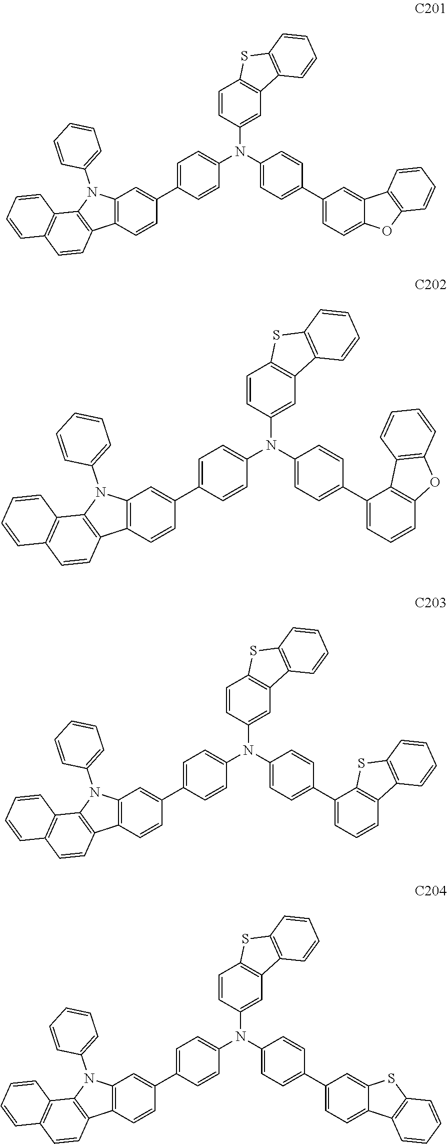 [SMILES: c1ccc(-n2c3cc(-c4ccc(N(c5ccc(-c6ccc7oc8ccccc8c7c6)cc5)c5ccc6sc7ccccc7c6c5)cc4)ccc3c3ccc4ccccc4c32)cc1 ; c1ccc(-n2c3cc(-c4ccc(N(c5ccc(-c6cccc7oc8ccccc8c67)cc5)c5ccc6sc7ccccc7c6c5)cc4)ccc3c3ccc4ccccc4c32)cc1 ; c1ccc(-n2c3cc(-c4ccc(N(c5ccc(-c6cccc7c6sc6ccccc67)cc5)c5ccc6sc7ccccc7c6c5)cc4)ccc3c3ccc4ccccc4c32)cc1 ; c1ccc(-n2c3cc(-c4ccc(N(c5ccc(-c6ccc7c(c6)sc6ccccc67)cc5)c5ccc6sc7ccccc7c6c5)cc4)ccc3c3ccc4ccccc4c32)cc1]

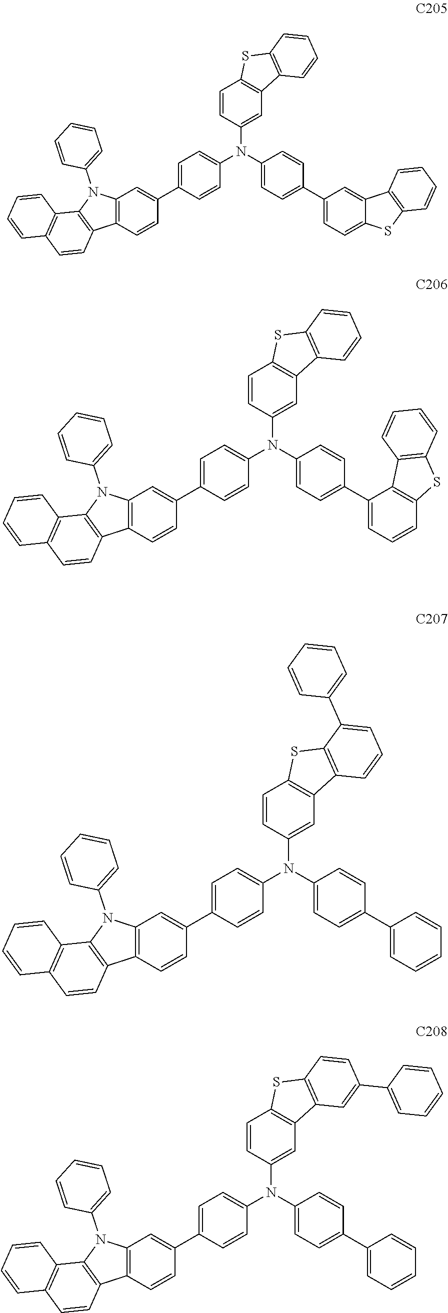 [SMILES: c1ccc(-n2c3cc(-c4ccc(N(c5ccc(-c6ccc7sc8ccccc8c7c6)cc5)c5ccc6sc7ccccc7c6c5)cc4)ccc3c3ccc4ccccc4c32)cc1 ; c1ccc(-n2c3cc(-c4ccc(N(c5ccc(-c6cccc7sc8ccccc8c67)cc5)c5ccc6sc7ccccc7c6c5)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4sc5c(-c6ccccc6)cccc5c4c3)cc2)cc1 ; c1ccc(-c2ccc(N(c3ccc(-c4ccc5c6ccc7ccccc7c6n(-c6ccccc6)c5c4)cc3)c3ccc4sc5ccc(-c6ccccc6)cc5c4c3)cc2)cc1]

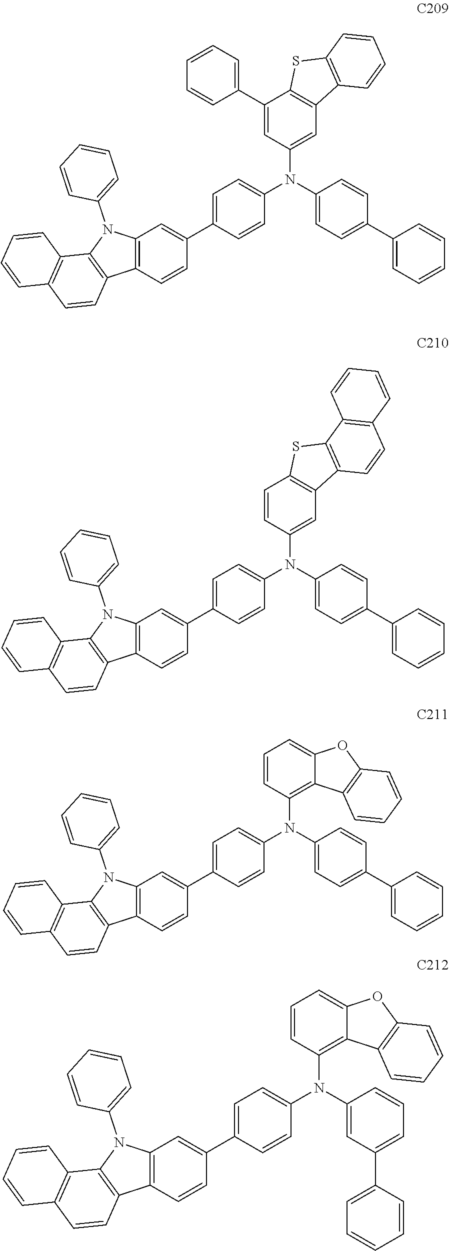 [SMILES: c1ccc(-c2ccc(N(c3ccc(-c4ccc5c6ccc7ccccc7c6n(-c6ccccc6)c5c4)cc3)c3cc(-c4ccccc4)c4sc5ccccc5c4c3)cc2)cc1 ; c1ccc(-c2ccc(N(c3ccc(-c4ccc5c6ccc7ccccc7c6n(-c6ccccc6)c5c4)cc3)c3ccc4sc5c6ccccc6ccc5c4c3)cc2)cc1 ; c1ccc(-c2ccc(N(c3ccc(-c4ccc5c6ccc7ccccc7c6n(-c6ccccc6)c5c4)cc3)c3cccc4oc5ccccc5c34)cc2)cc1 ; c1ccc(-c2cccc(N(c3ccc(-c4ccc5c6ccc7ccccc7c6n(-c6ccccc6)c5c4)cc3)c3cccc4oc5ccccc5c34)c2)cc1]

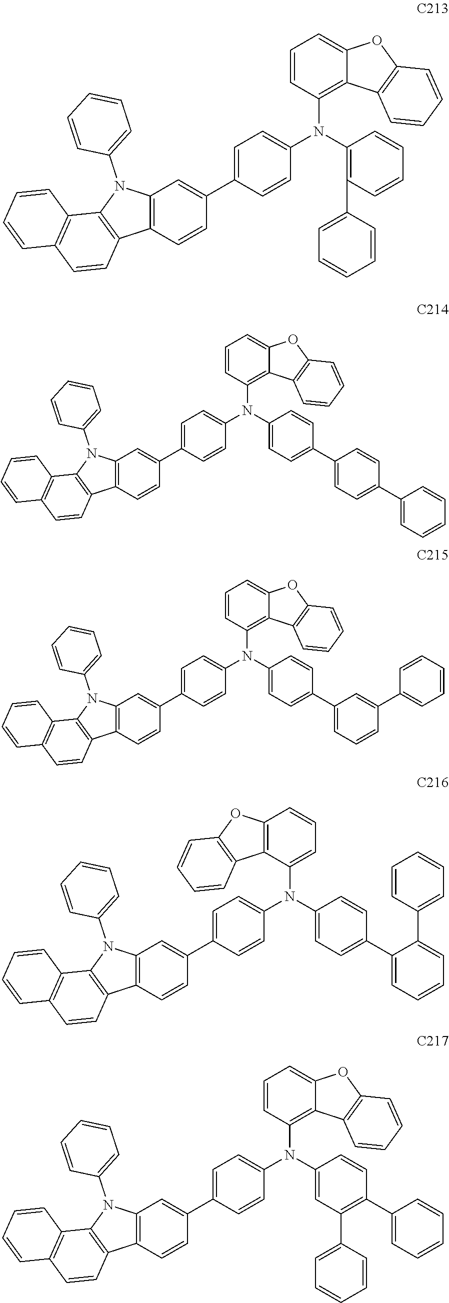 [SMILES: c1ccc(-c2ccccc2N(c2ccc(-c3ccc4c5ccc6ccccc6c5n(-c5ccccc5)c4c3)cc2)c2cccc3oc4ccccc4c23)cc1 ; c1ccc(-c2ccc(-c3ccc(N(c4ccc(-c5ccc6c7ccc8ccccc8c7n(-c7ccccc7)c6c5)cc4)c4cccc5oc6ccccc6c45)cc3)cc2)cc1 ; c1ccc(-c2cccc(-c3ccc(N(c4ccc(-c5ccc6c7ccc8ccccc8c7n(-c7ccccc7)c6c5)cc4)c4cccc5oc6ccccc6c45)cc3)c2)cc1 ; c1ccc(-c2ccccc2-c2ccc(N(c3ccc(-c4ccc5c6ccc7ccccc7c6n(-c6ccccc6)c5c4)cc3)c3cccc4oc5ccccc5c34)cc2)cc1 ; c1ccc(-c2ccc(N(c3ccc(-c4ccc5c6ccc7ccccc7c6n(-c6ccccc6)c5c4)cc3)c3cccc4oc5ccccc5c34)cc2-c2ccccc2)cc1]

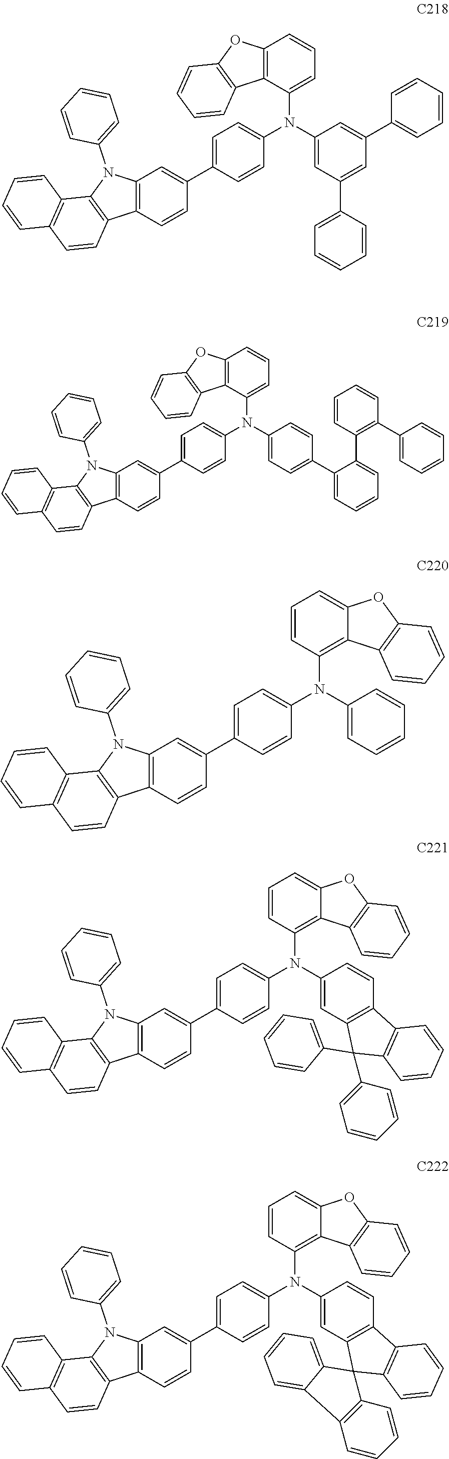

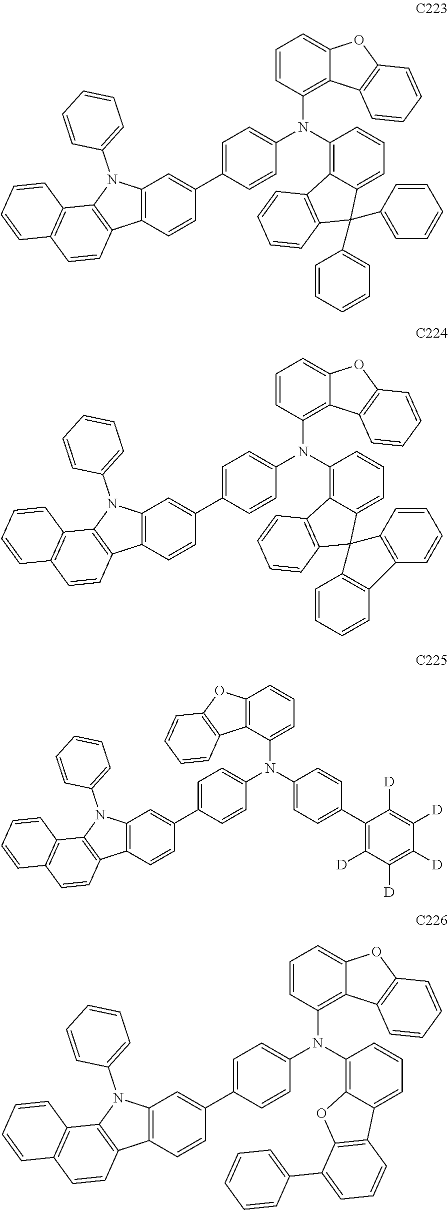 [SMILES: c1ccc(-n2c3cc(-c4ccc(N(c5cccc6c5-c5ccccc5C6(c5ccccc5)c5ccccc5)c5cccc6oc7ccccc7c56)cc4)ccc3c3ccc4ccccc4c32)cc1 ; c1ccc(-n2c3cc(-c4ccc(N(c5cccc6c5-c5ccccc5C65c6ccccc6-c6ccccc65)c5cccc6oc7ccccc7c56)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3cccc4oc5ccccc5c34)cc2)c([2H])c1[2H] ; c1ccc(-c2cccc3c2oc2c(N(c4ccc(-c5ccc6c7ccc8ccccc8c7n(-c7ccccc7)c6c5)cc4)c4cccc5oc6ccccc6c45)cccc23)cc1]

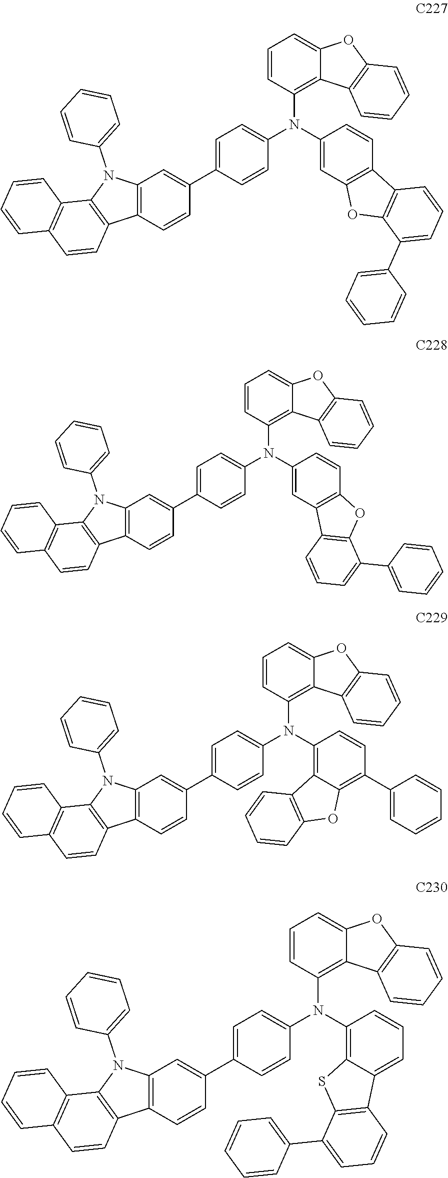 [SMILES: c1ccc(-c2cccc3c2oc2cc(N(c4ccc(-c5ccc6c7ccc8ccccc8c7n(-c7ccccc7)c6c5)cc4)c4cccc5oc6ccccc6c45)ccc23)cc1 ; c1ccc(-c2cccc3c2oc2ccc(N(c4ccc(-c5ccc6c7ccc8ccccc8c7n(-c7ccccc7)c6c5)cc4)c4cccc5oc6ccccc6c45)cc23)cc1 ; c1ccc(-c2ccc(N(c3ccc(-c4ccc5c6ccc7ccccc7c6n(-c6ccccc6)c5c4)cc3)c3cccc4oc5ccccc5c34)c3c2oc2ccccc23)cc1 ; c1ccc(-c2cccc3c2sc2c(N(c4ccc(-c5ccc6c7ccc8ccccc8c7n(-c7ccccc7)c6c5)cc4)c4cccc5oc6ccccc6c45)cccc23)cc1]

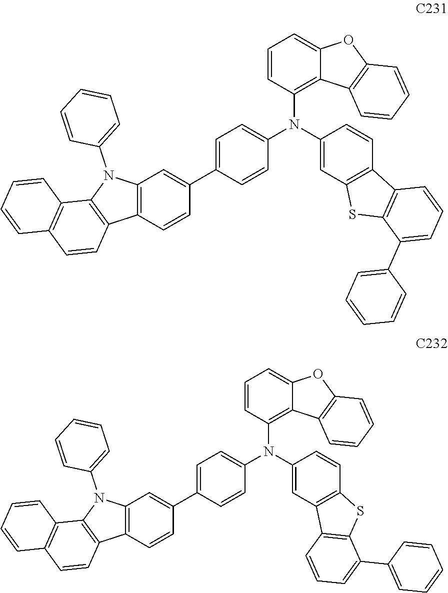 [SMILES: c1ccc(-c2cccc3c2sc2cc(N(c4ccc(-c5ccc6c7ccc8ccccc8c7n(-c7ccccc7)c6c5)cc4)c4cccc5oc6ccccc6c45)ccc23)cc1 ; c1ccc(-c2cccc3c2sc2ccc(N(c4ccc(-c5ccc6c7ccc8ccccc8c7n(-c7ccccc7)c6c5)cc4)c4cccc5oc6ccccc6c45)cc23)cc1]

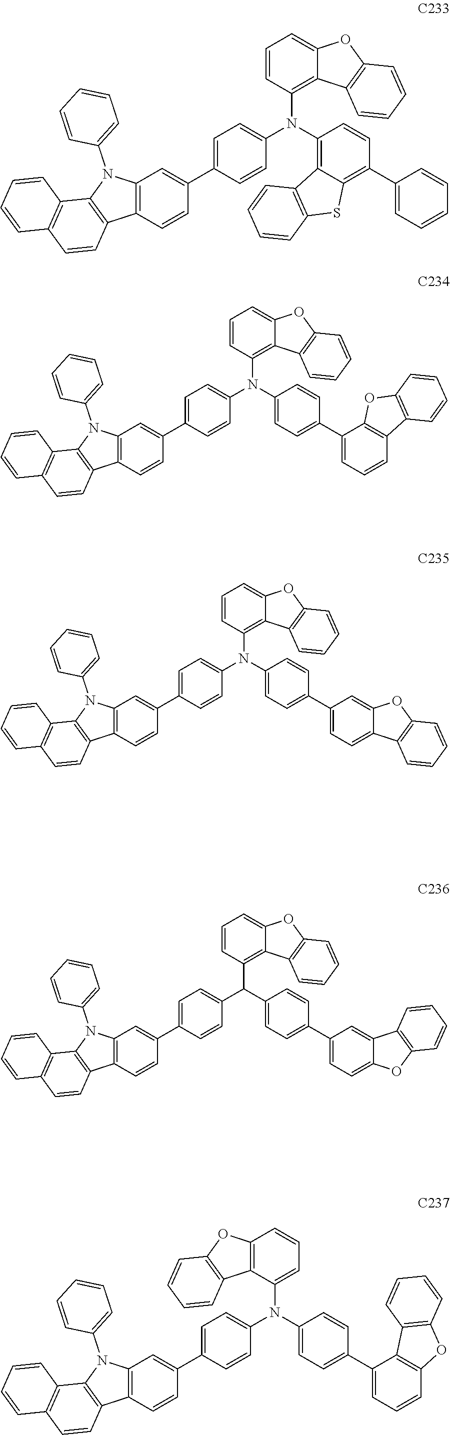

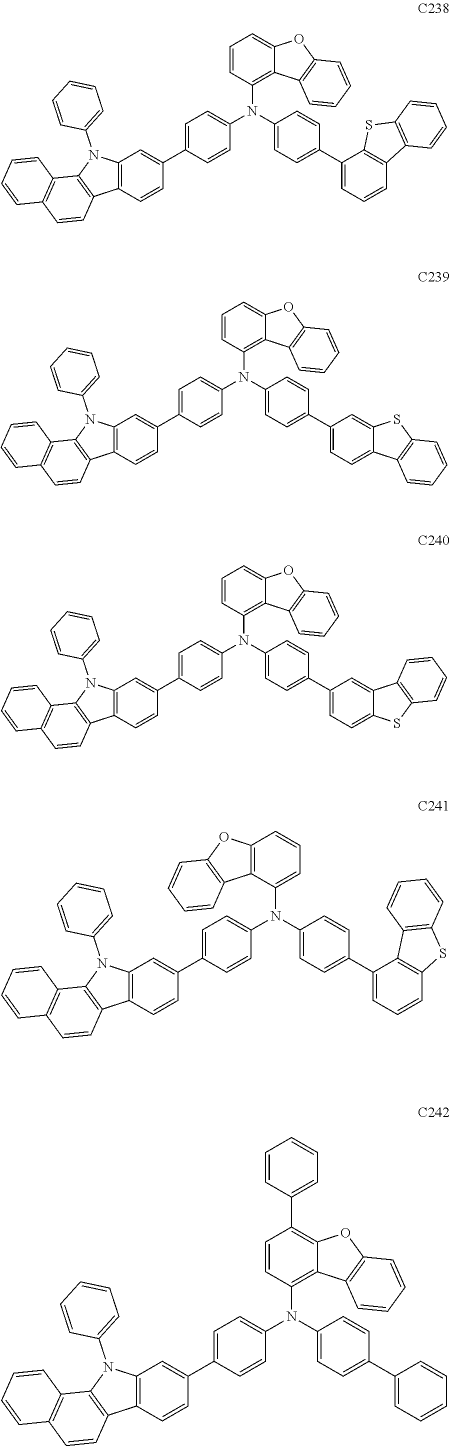

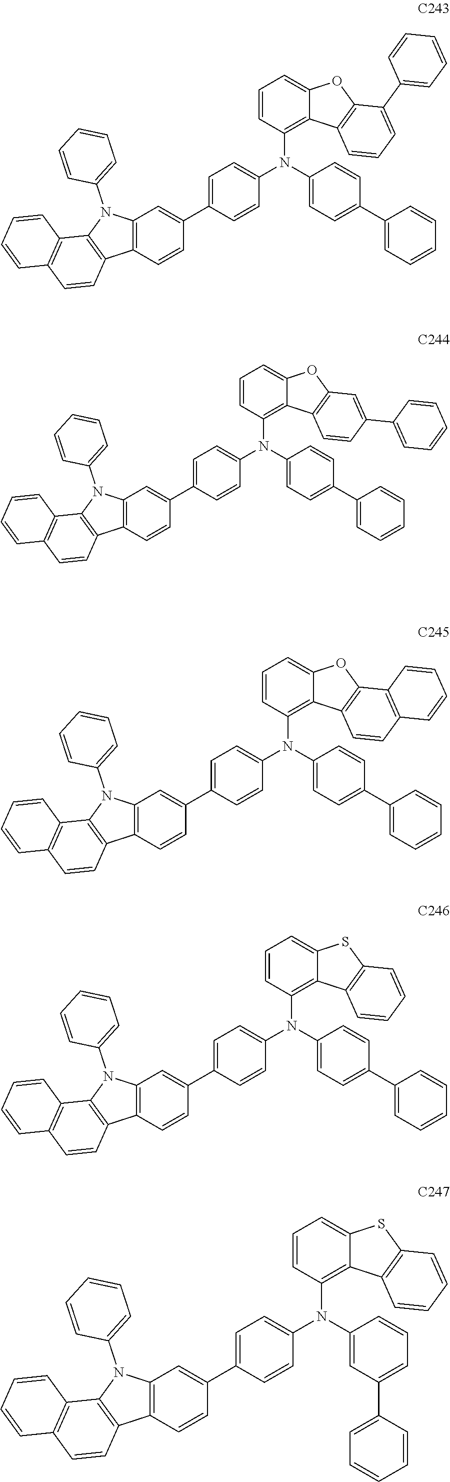 [SMILES: c1ccc(-c2ccc(N(c3ccc(-c4ccc5c6ccc7ccccc7c6n(-c6ccccc6)c5c4)cc3)c3cccc4oc5c(-c6ccccc6)cccc5c34)cc2)cc1 ; c1ccc(-c2ccc(N(c3ccc(-c4ccc5c6ccc7ccccc7c6n(-c6ccccc6)c5c4)cc3)c3cccc4oc5cc(-c6ccccc6)ccc5c34)cc2)cc1 ; c1ccc(-c2ccc(N(c3ccc(-c4ccc5c6ccc7ccccc7c6n(-c6ccccc6)c5c4)cc3)c3cccc4oc5c6ccccc6ccc5c34)cc2)cc1 ; c1ccc(-c2ccc(N(c3ccc(-c4ccc5c6ccc7ccccc7c6n(-c6ccccc6)c5c4)cc3)c3cccc4sc5ccccc5c34)cc2)cc1 ; c1ccc(-c2cccc(N(c3ccc(-c4ccc5c6ccc7ccccc7c6n(-c6ccccc6)c5c4)cc3)c3cccc4sc5ccccc5c34)c2)cc1]

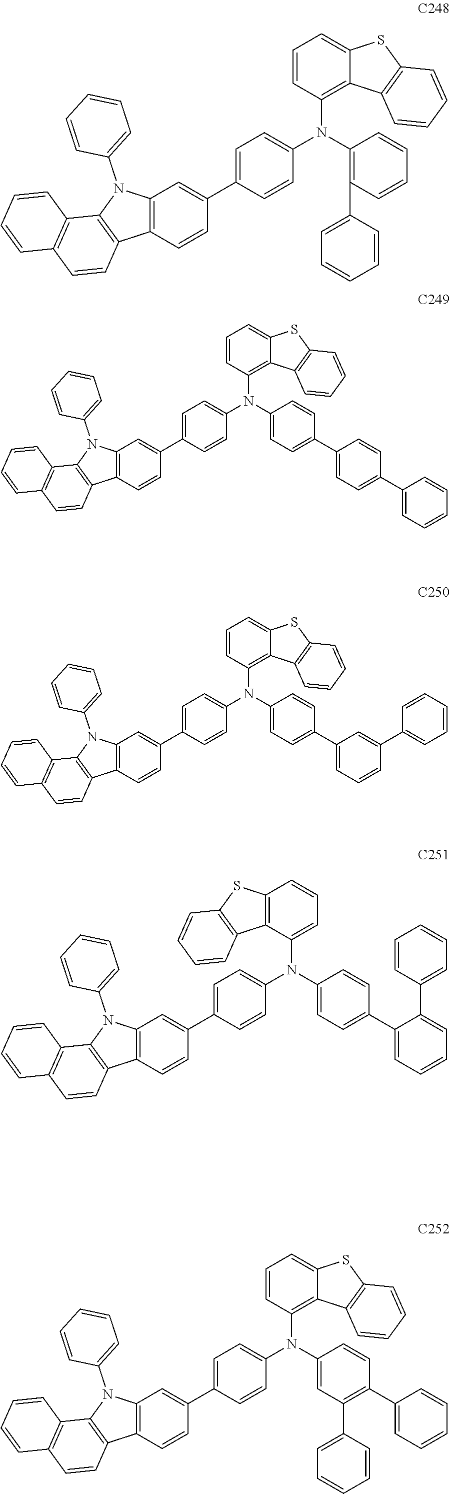 [SMILES: c1ccc(-c2ccccc2N(c2ccc(-c3ccc4c5ccc6ccccc6c5n(-c5ccccc5)c4c3)cc2)c2cccc3sc4ccccc4c23)cc1 ; c1ccc(-c2ccc(-c3ccc(N(c4ccc(-c5ccc6c7ccc8ccccc8c7n(-c7ccccc7)c6c5)cc4)c4cccc5sc6ccccc6c45)cc3)cc2)cc1 ; c1ccc(-c2cccc(-c3ccc(N(c4ccc(-c5ccc6c7ccc8ccccc8c7n(-c7ccccc7)c6c5)cc4)c4cccc5sc6ccccc6c45)cc3)c2)cc1 ; c1ccc(-c2ccccc2-c2ccc(N(c3ccc(-c4ccc5c6ccc7ccccc7c6n(-c6ccccc6)c5c4)cc3)c3cccc4sc5ccccc5c34)cc2)cc1 ; c1ccc(-c2ccc(N(c3ccc(-c4ccc5c6ccc7ccccc7c6n(-c6ccccc6)c5c4)cc3)c3cccc4sc5ccccc5c34)cc2-c2ccccc2)cc1]

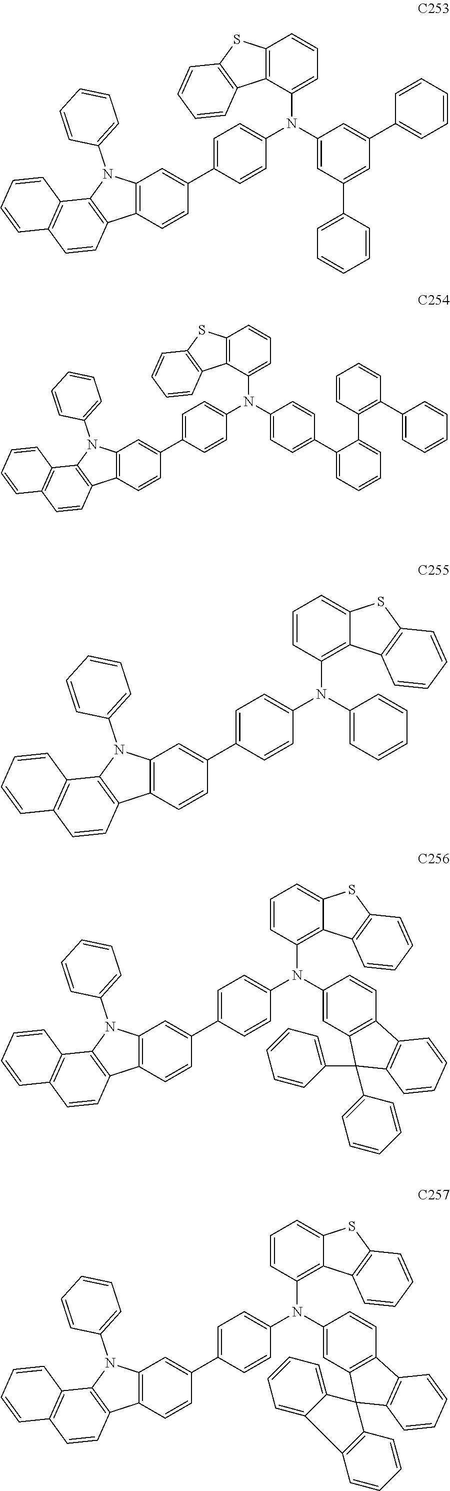

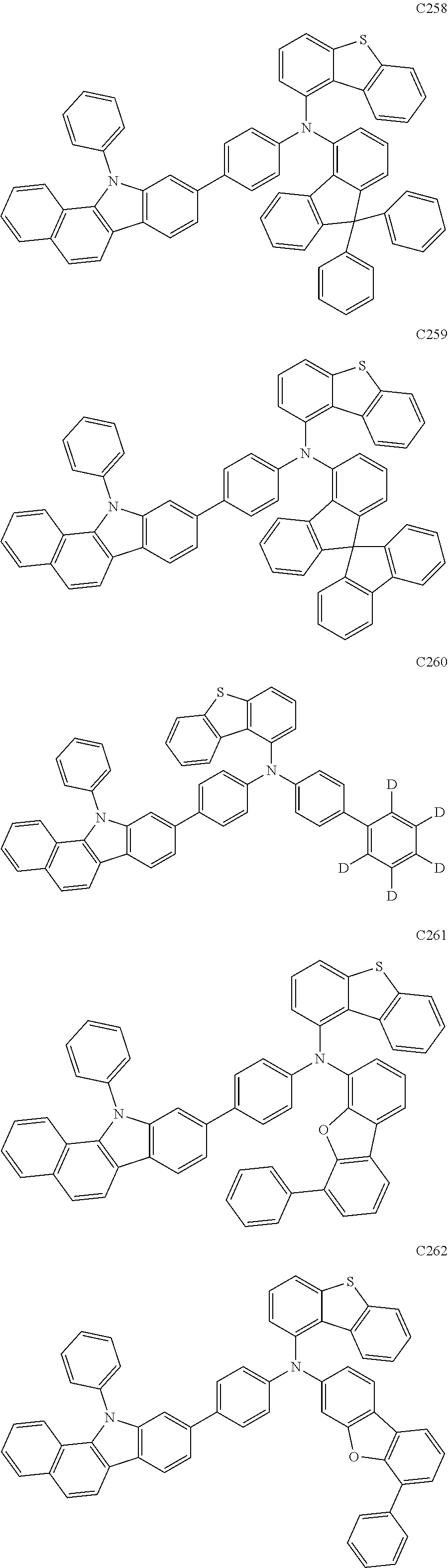

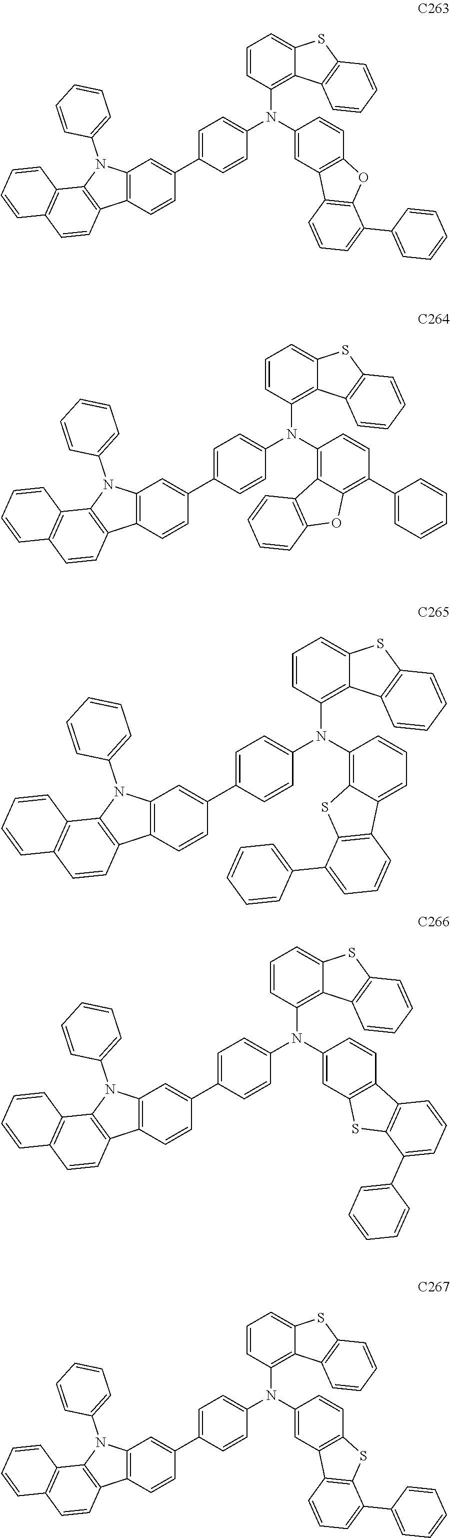

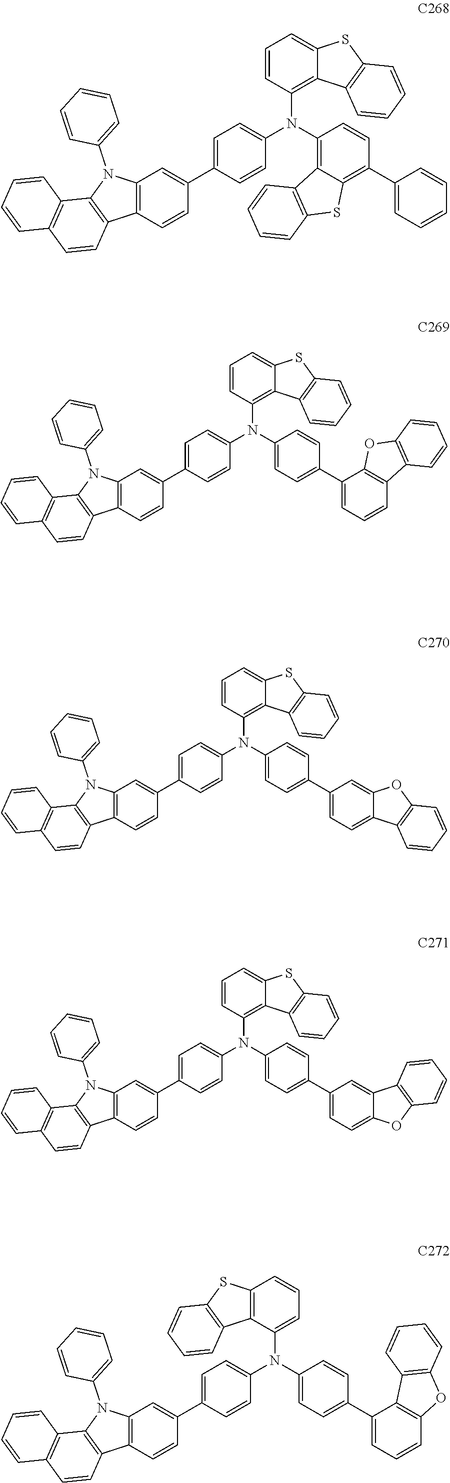

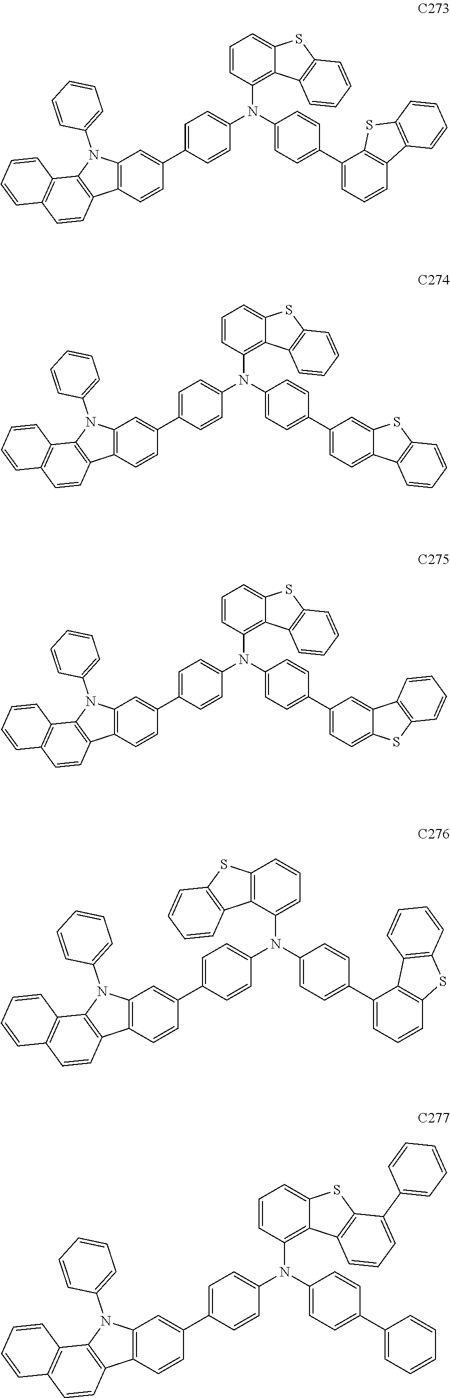

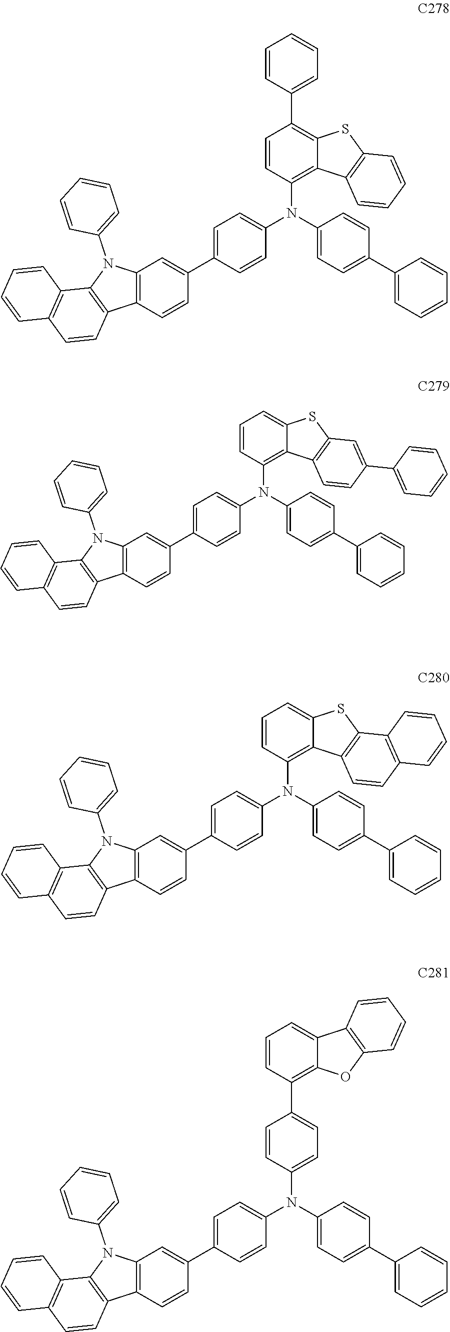 [SMILES: c1ccc(-c2ccc(N(c3ccc(-c4ccc5c6ccc7ccccc7c6n(-c6ccccc6)c5c4)cc3)c3ccc(-c4ccccc4)c4sc5ccccc5c34)cc2)cc1 ; c1ccc(-c2ccc(N(c3ccc(-c4ccc5c6ccc7ccccc7c6n(-c6ccccc6)c5c4)cc3)c3cccc4sc5cc(-c6ccccc6)ccc5c34)cc2)cc1 ; c1ccc(-c2ccc(N(c3ccc(-c4ccc5c6ccc7ccccc7c6n(-c6ccccc6)c5c4)cc3)c3cccc4sc5c6ccccc6ccc5c34)cc2)cc1 ; c1ccc(-c2ccc(N(c3ccc(-c4ccc5c6ccc7ccccc7c6n(-c6ccccc6)c5c4)cc3)c3ccc(-c4cccc5c4oc4ccccc45)cc3)cc2)cc1]

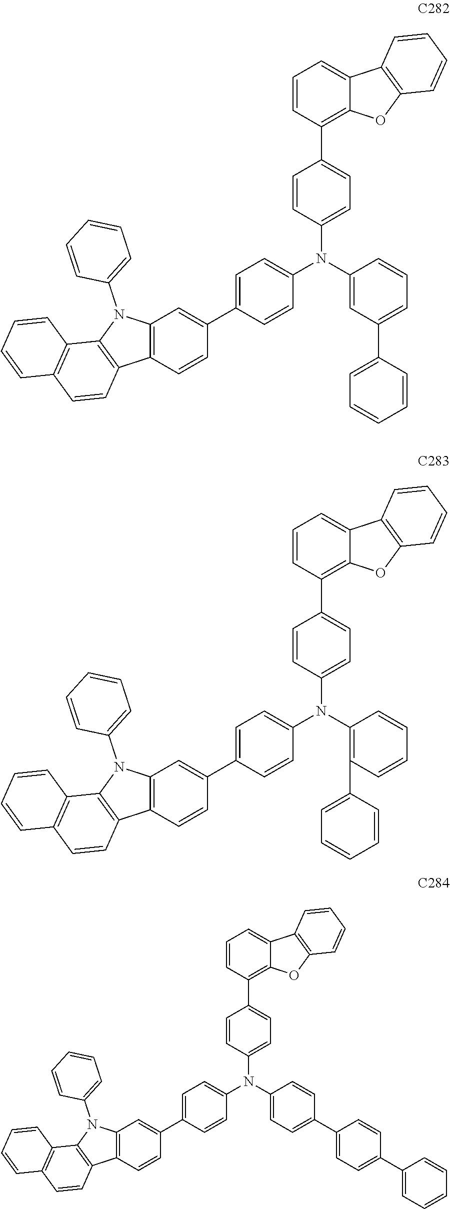 [SMILES: c1ccc(-c2cccc(N(c3ccc(-c4ccc5c6ccc7ccccc7c6n(-c6ccccc6)c5c4)cc3)c3ccc(-c4cccc5c4oc4ccccc45)cc3)c2)cc1 ; c1ccc(-c2ccccc2N(c2ccc(-c3ccc4c5ccc6ccccc6c5n(-c5ccccc5)c4c3)cc2)c2ccc(-c3cccc4c3oc3ccccc34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6c5oc5ccccc56)cc4)cc3)cc2)cc1]

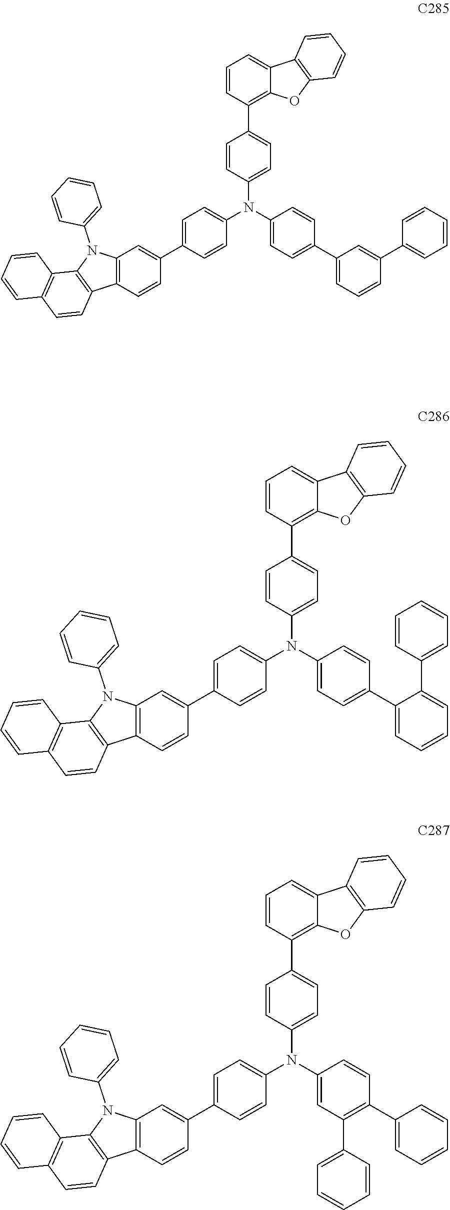 [SMILES: c1ccc(-c2cccc(-c3ccc(N(c4ccc(-c5ccc6c7ccc8ccccc8c7n(-c7ccccc7)c6c5)cc4)c4ccc(-c5cccc6c5oc5ccccc56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5c4oc4ccccc45)cc3)cc2)cc1 ; c1ccc(-c2ccc(N(c3ccc(-c4ccc5c6ccc7ccccc7c6n(-c6ccccc6)c5c4)cc3)c3ccc(-c4cccc5c4oc4ccccc45)cc3)cc2-c2ccccc2)cc1]

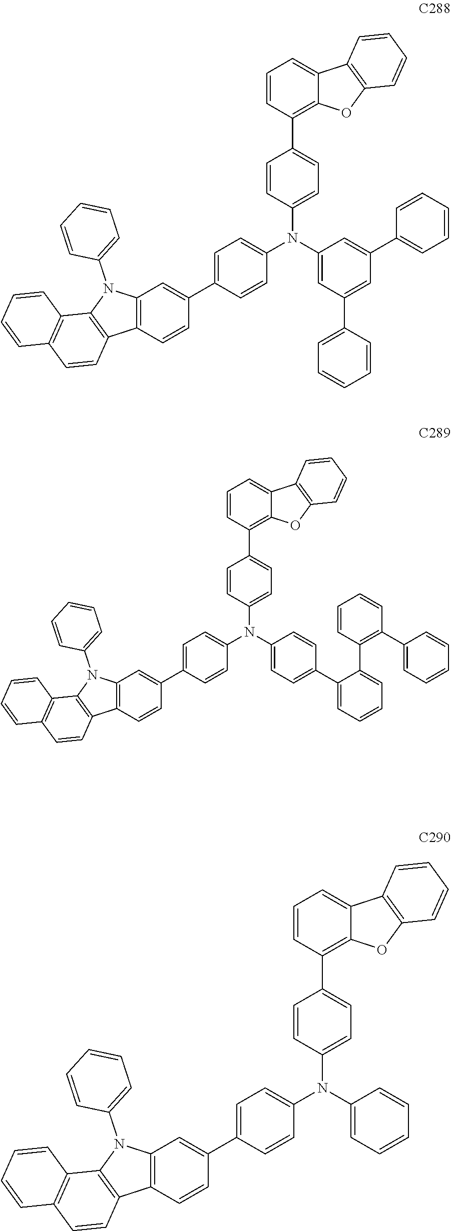 [SMILES: c1ccc(-c2cc(-c3ccccc3)cc(N(c3ccc(-c4ccc5c6ccc7ccccc7c6n(-c6ccccc6)c5c4)cc3)c3ccc(-c4cccc5c4oc4ccccc45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5c4oc4ccccc45)cc3)cc2)cc1 ; c1ccc(N(c2ccc(-c3ccc4c5ccc6ccccc6c5n(-c5ccccc5)c4c3)cc2)c2ccc(-c3cccc4c3oc3ccccc34)cc2)cc1]

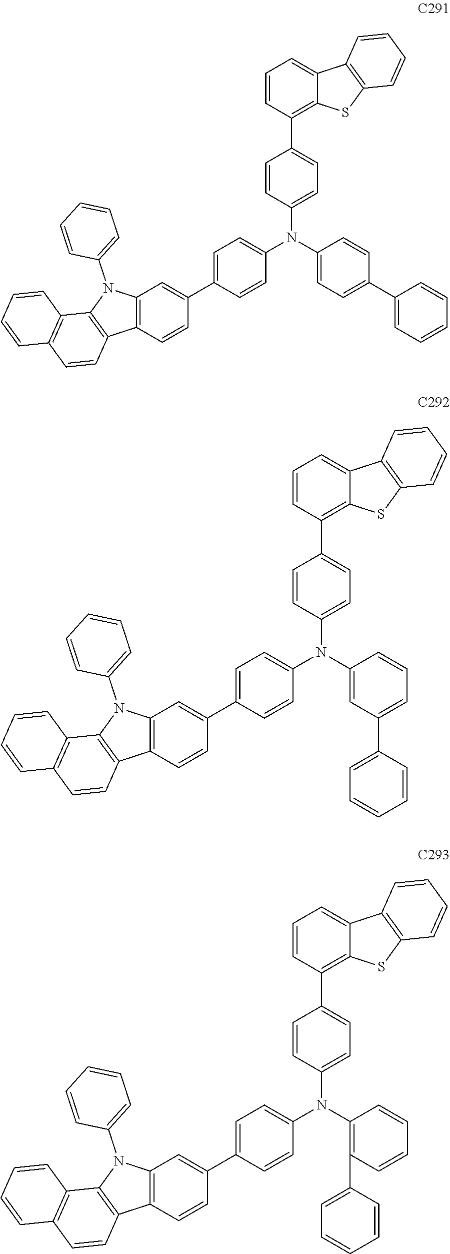 [SMILES: c1ccc(-c2ccc(N(c3ccc(-c4ccc5c6ccc7ccccc7c6n(-c6ccccc6)c5c4)cc3)c3ccc(-c4cccc5c4sc4ccccc45)cc3)cc2)cc1 ; c1ccc(-c2cccc(N(c3ccc(-c4ccc5c6ccc7ccccc7c6n(-c6ccccc6)c5c4)cc3)c3ccc(-c4cccc5c4sc4ccccc45)cc3)c2)cc1 ; c1ccc(-c2ccccc2N(c2ccc(-c3ccc4c5ccc6ccccc6c5n(-c5ccccc5)c4c3)cc2)c2ccc(-c3cccc4c3sc3ccccc34)cc2)cc1]

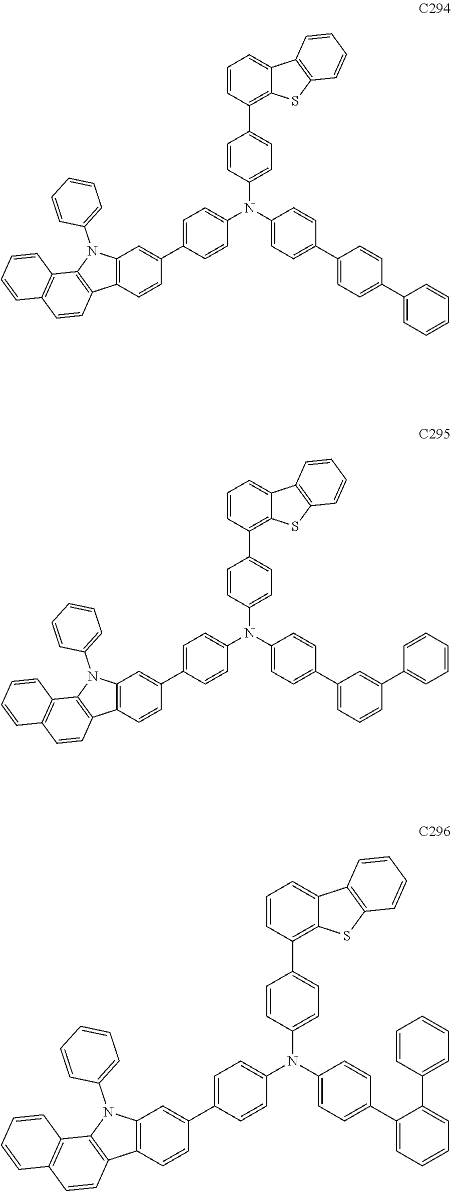 [SMILES: c1ccc(-c2ccc(-c3ccc(N(c4ccc(-c5ccc6c7ccc8ccccc8c7n(-c7ccccc7)c6c5)cc4)c4ccc(-c5cccc6c5sc5ccccc56)cc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc(-c5cccc6c5sc5ccccc56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5c4sc4ccccc45)cc3)cc2)cc1]

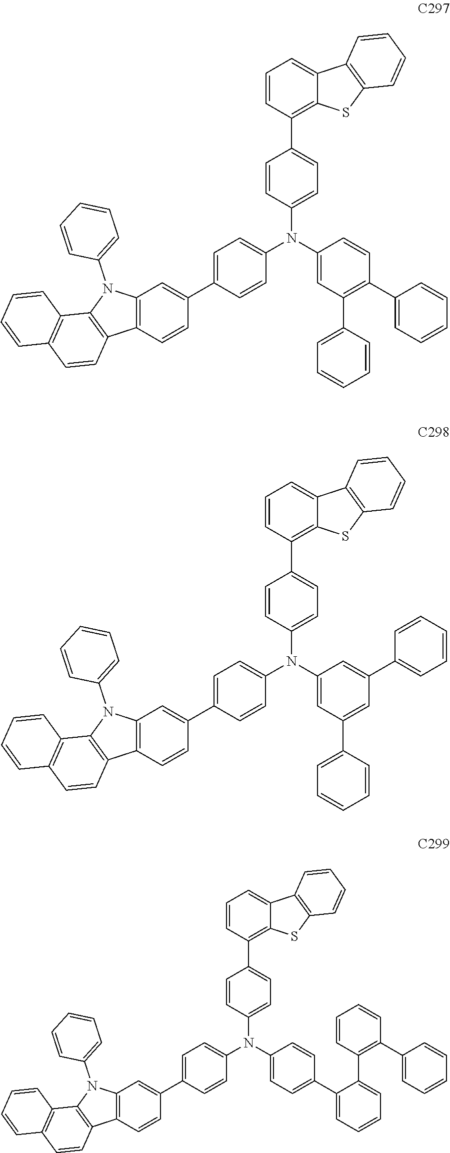 [SMILES: c1ccc(-c2ccc(N(c3ccc(-c4ccc5c6ccc7ccccc7c6n(-c6ccccc6)c5c4)cc3)c3ccc(-c4cccc5c4sc4ccccc45)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cccc5c4sc4ccccc45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5c4sc4ccccc45)cc3)cc2)cc1]

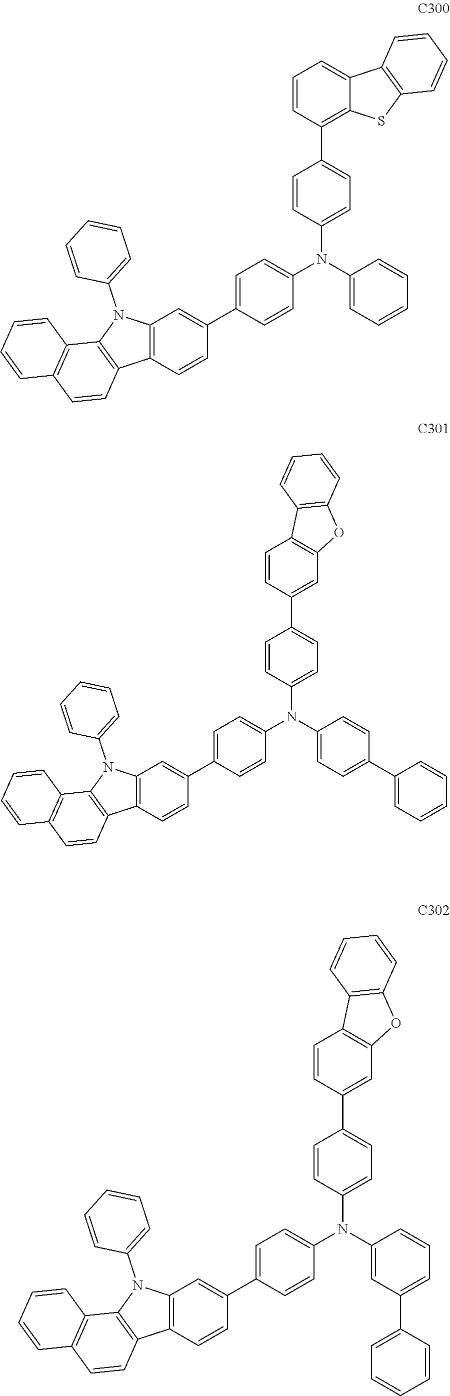 [SMILES: c1ccc(N(c2ccc(-c3ccc4c5ccc6ccccc6c5n(-c5ccccc5)c4c3)cc2)c2ccc(-c3cccc4c3sc3ccccc34)cc2)cc1 ; c1ccc(-c2ccc(N(c3ccc(-c4ccc5c(c4)oc4ccccc45)cc3)c3ccc(-c4ccc5c6ccc7ccccc7c6n(-c6ccccc6)c5c4)cc3)cc2)cc1 ; c1ccc(-c2cccc(N(c3ccc(-c4ccc5c(c4)oc4ccccc45)cc3)c3ccc(-c4ccc5c6ccc7ccccc7c6n(-c6ccccc6)c5c4)cc3)c2)cc1]

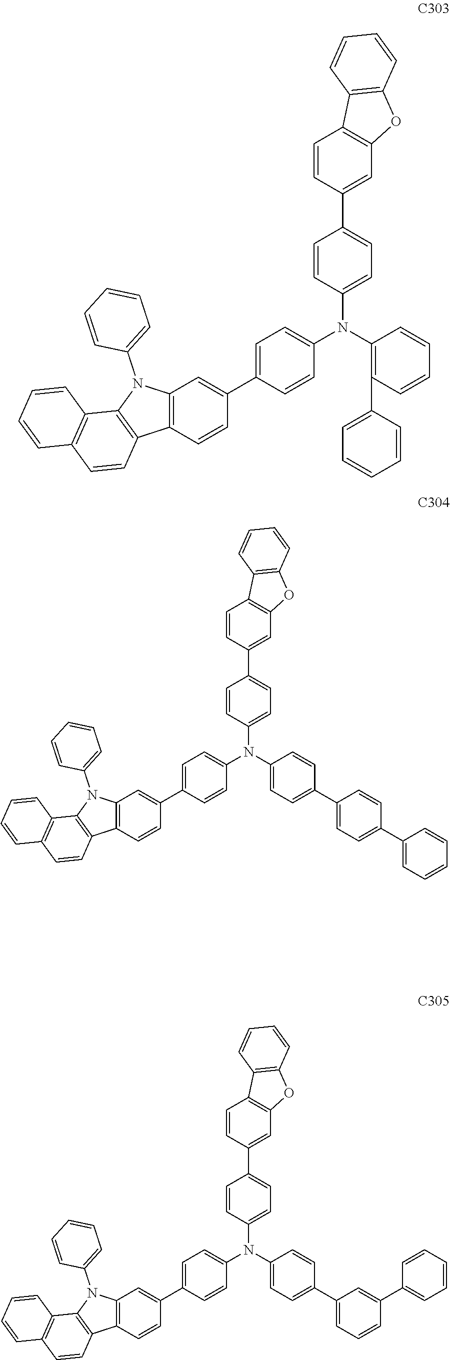 [SMILES: c1ccc(-c2ccccc2N(c2ccc(-c3ccc4c(c3)oc3ccccc34)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6c(c5)oc5ccccc56)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6c(c5)oc5ccccc56)cc4)c4ccc(-c5ccc6c7ccc8ccccc8c7n(-c7ccccc7)c6c5)cc4)cc3)c2)cc1]

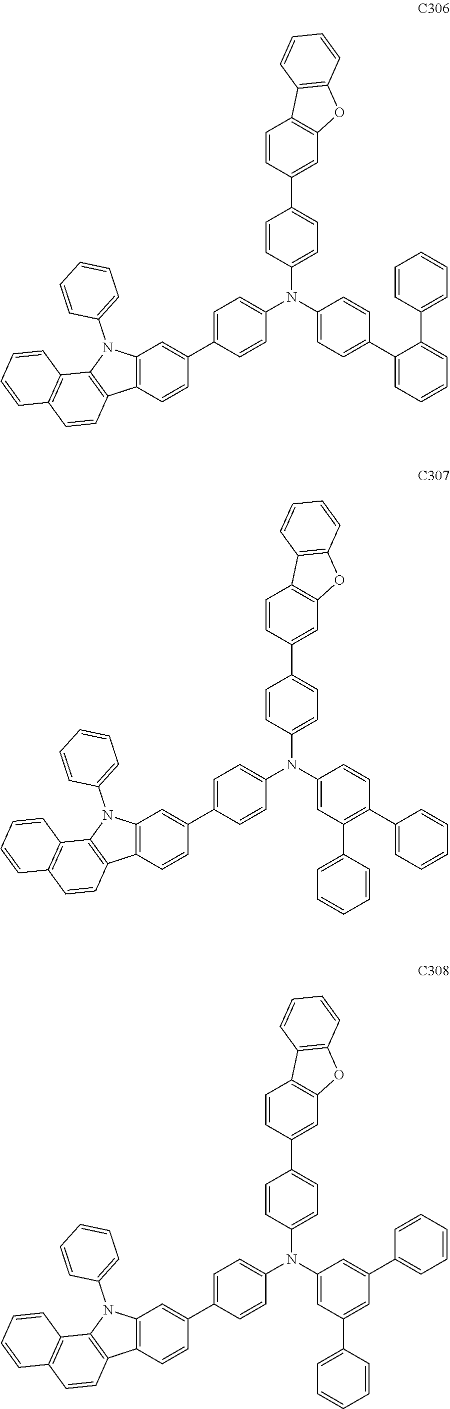 [SMILES: c1ccc(-c2ccccc2-c2ccc(N(c3ccc(-c4ccc5c(c4)oc4ccccc45)cc3)c3ccc(-c4ccc5c6ccc7ccccc7c6n(-c6ccccc6)c5c4)cc3)cc2)cc1 ; c1ccc(-c2ccc(N(c3ccc(-c4ccc5c(c4)oc4ccccc45)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5c(c4)oc4ccccc45)cc3)c3ccc(-c4ccc5c6ccc7ccccc7c6n(-c6ccccc6)c5c4)cc3)c2)cc1]

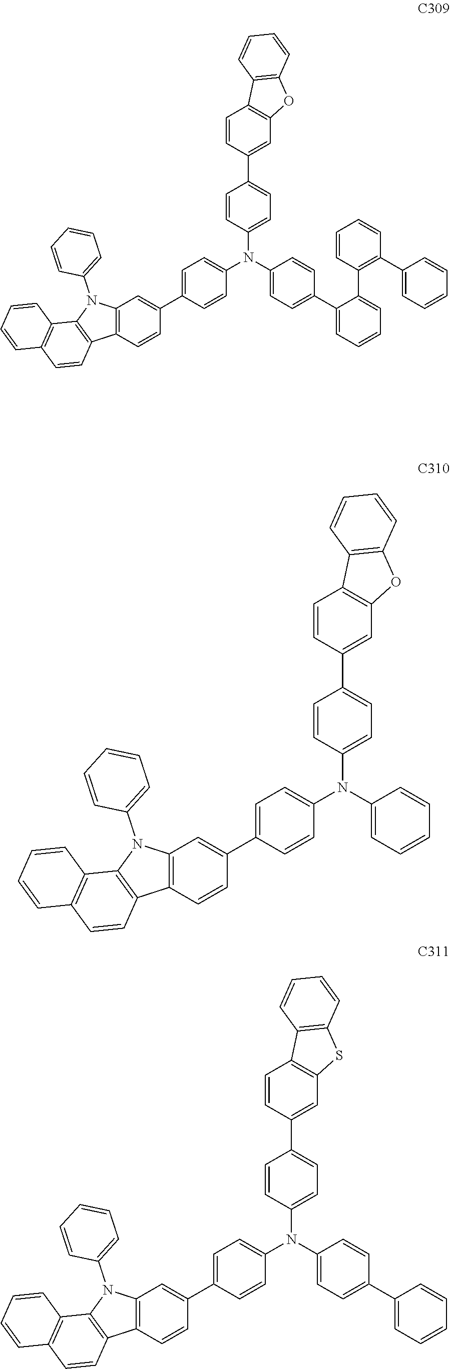 [SMILES: c1ccc(-c2ccccc2-c2ccccc2-c2ccc(N(c3ccc(-c4ccc5c(c4)oc4ccccc45)cc3)c3ccc(-c4ccc5c6ccc7ccccc7c6n(-c6ccccc6)c5c4)cc3)cc2)cc1 ; c1ccc(N(c2ccc(-c3ccc4c(c3)oc3ccccc34)cc2)c2ccc(-c3ccc4c5ccc6ccccc6c5n(-c5ccccc5)c4c3)cc2)cc1 ; c1ccc(-c2ccc(N(c3ccc(-c4ccc5c(c4)sc4ccccc45)cc3)c3ccc(-c4ccc5c6ccc7ccccc7c6n(-c6ccccc6)c5c4)cc3)cc2)cc1]

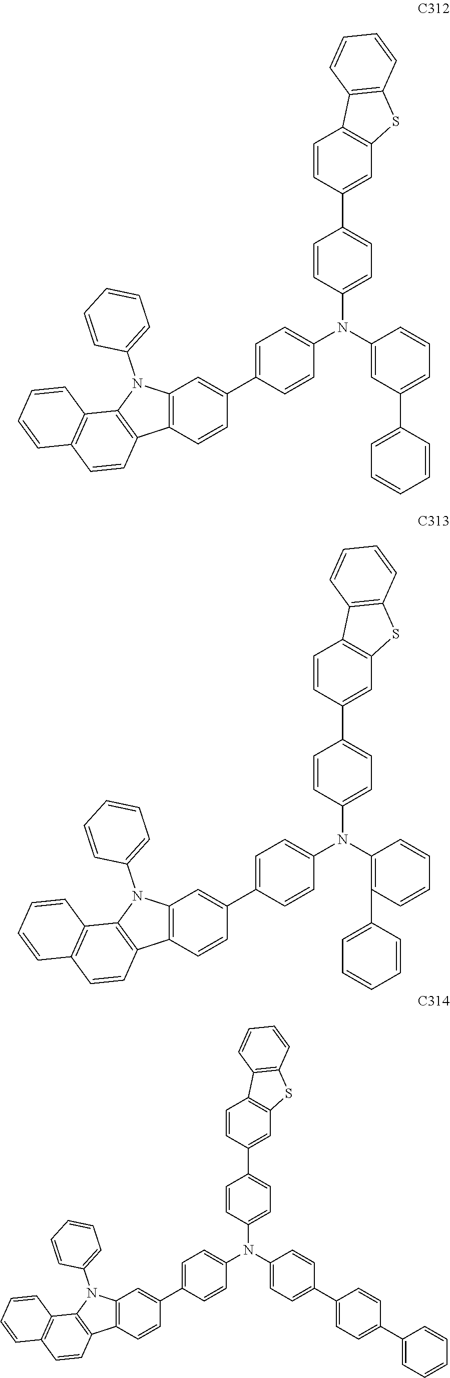 [SMILES: c1ccc(-c2cccc(N(c3ccc(-c4ccc5c(c4)sc4ccccc45)cc3)c3ccc(-c4ccc5c6ccc7ccccc7c6n(-c6ccccc6)c5c4)cc3)c2)cc1 ; c1ccc(-c2ccccc2N(c2ccc(-c3ccc4c(c3)sc3ccccc34)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6c(c5)sc5ccccc56)cc4)c4ccc(-c5ccc6c7ccc8ccccc8c7n(-c7ccccc7)c6c5)cc4)cc3)cc2)cc1]

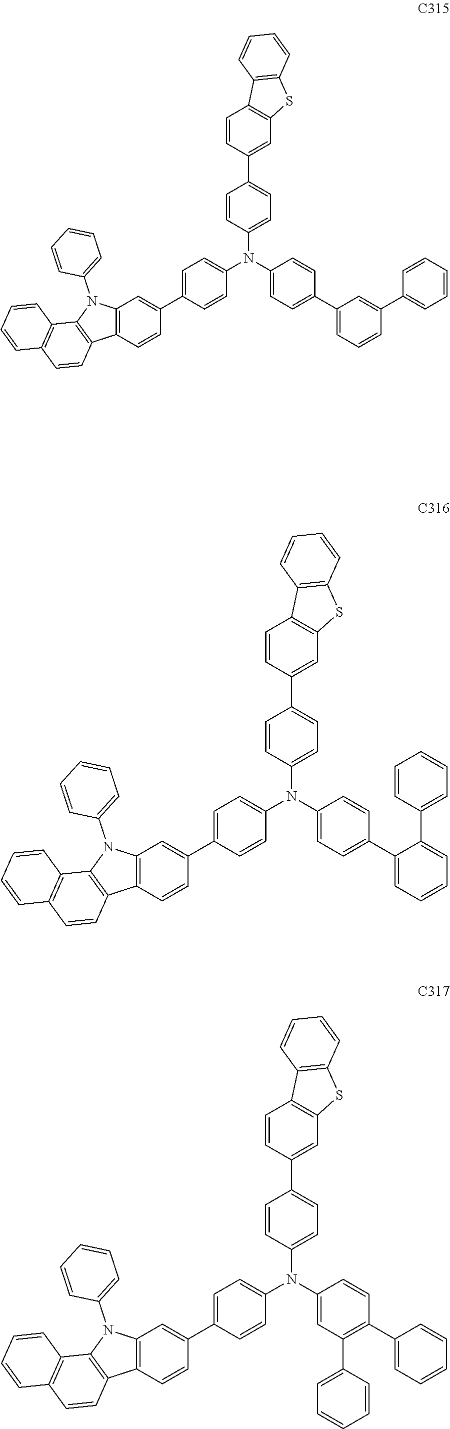 [SMILES: c1ccc(-c2cccc(-c3ccc(N(c4ccc(-c5ccc6c(c5)sc5ccccc56)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5c(c4)sc4ccccc45)cc3)c3ccc(-c4ccc5c6ccc7ccccc7c6n(-c6ccccc6)c5c4)cc3)cc2)cc1 ; c1ccc(-c2ccc(N(c3ccc(-c4ccc5c(c4)sc4ccccc45)cc3)c3ccc(-c4ccc5c6ccc7ccccc7c6n(-c6ccccc6)c5c4)cc3)cc2-c2ccccc2)cc1]

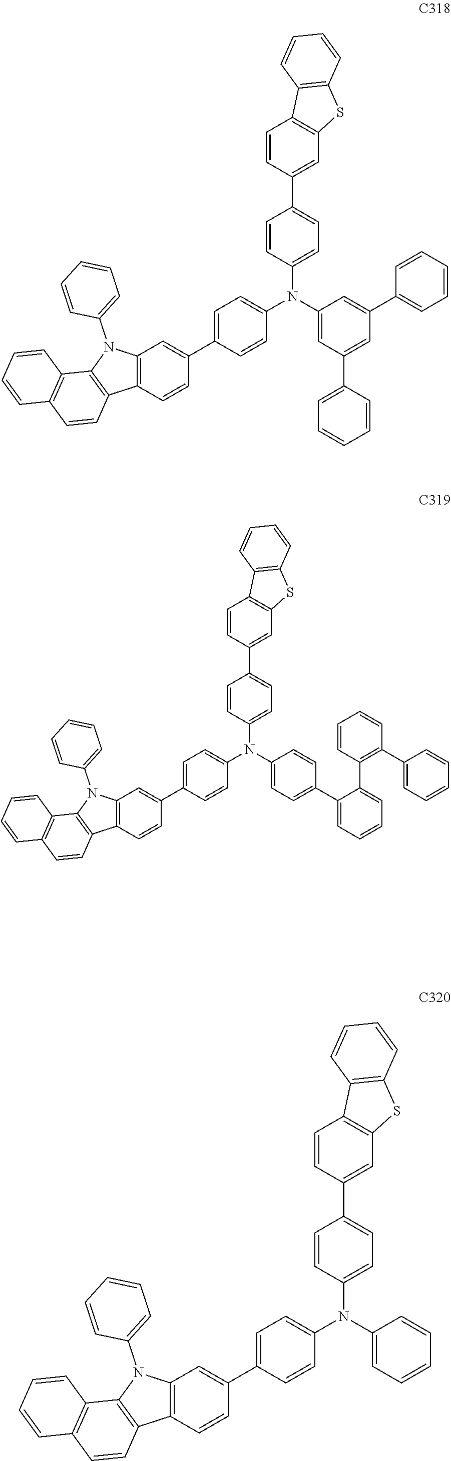 [SMILES: c1ccc(-c2cc(-c3ccccc3)cc(N(c3ccc(-c4ccc5c(c4)sc4ccccc45)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5c(c4)sc4ccccc45)cc3)c3ccc(-c4ccc5c6ccc7ccccc7c6n(-c6ccccc6)c5c4)cc3)cc2)cc1 ; c1ccc(N(c2ccc(-c3ccc4c(c3)sc3ccccc34)cc2)c2ccc(-c3ccc4c5ccc6ccccc6c5n(-c5ccccc5)c4c3)cc2)cc1]

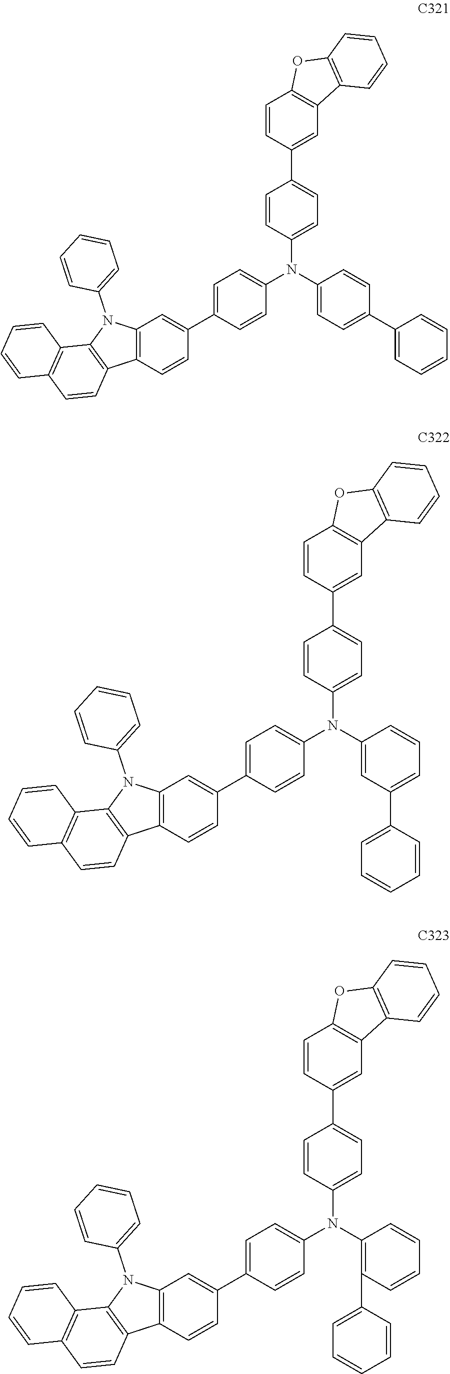 [SMILES: c1ccc(-c2ccc(N(c3ccc(-c4ccc5oc6ccccc6c5c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5oc6ccccc6c5c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4oc5ccccc5c4c3)cc2)c2ccc(-c3ccc4c5ccc6ccccc6c5n(-c5ccccc5)c4c3)cc2)cc1]

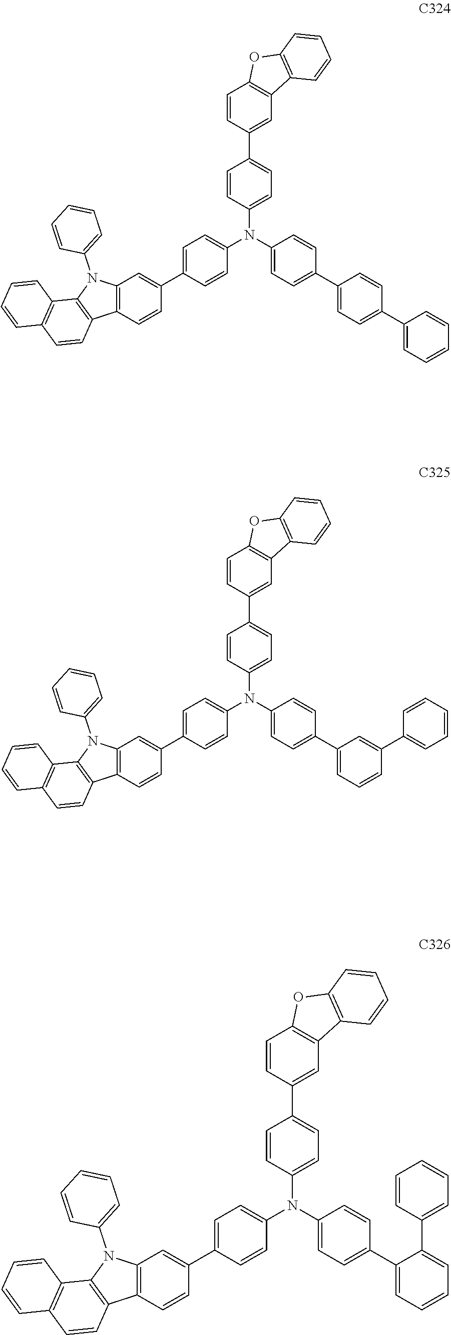 [SMILES: c1ccc(-c2ccc(-c3ccc(N(c4ccc(-c5ccc6oc7ccccc7c6c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6oc7ccccc7c6c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5oc6ccccc6c5c4)cc3)c3ccc(-c4ccc5c6ccc7ccccc7c6n(-c6ccccc6)c5c4)cc3)cc2)cc1]

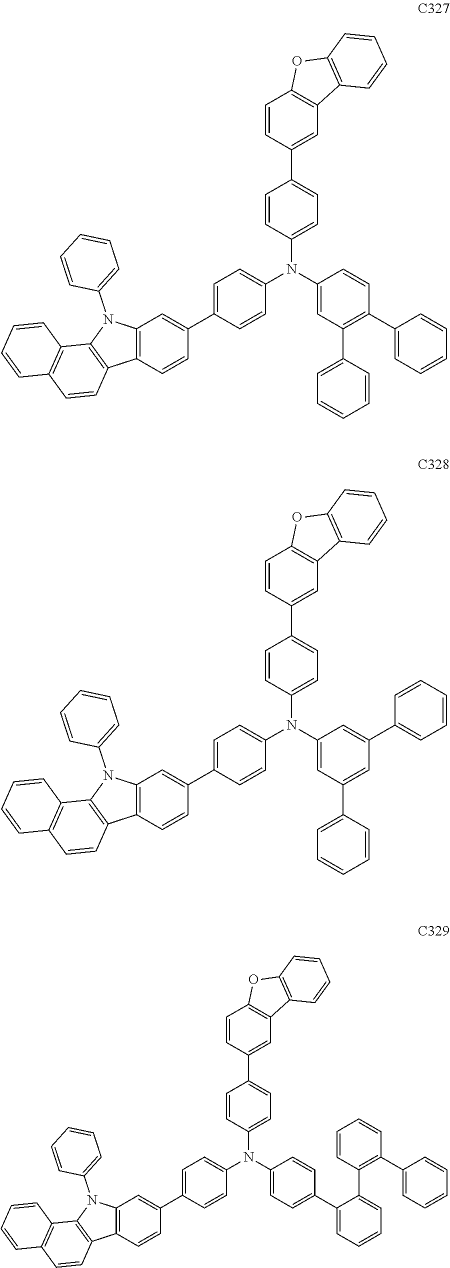 [SMILES: c1ccc(-c2ccc(N(c3ccc(-c4ccc5oc6ccccc6c5c4)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5oc6ccccc6c5c4)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5oc6ccccc6c5c4)cc3)c3ccc(-c4ccc5c6ccc7ccccc7c6n(-c6ccccc6)c5c4)cc3)cc2)cc1]

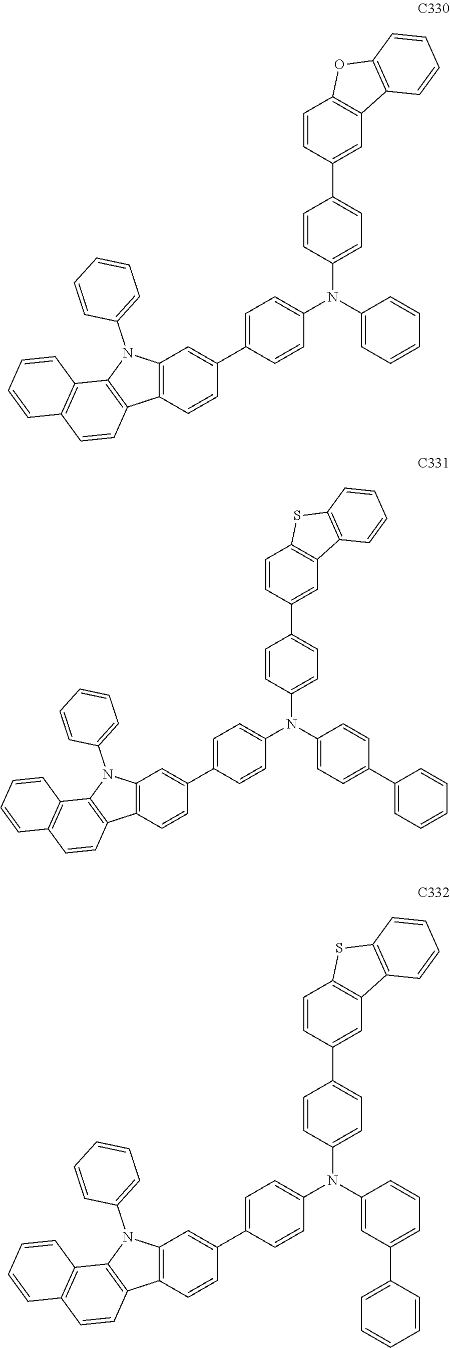 [SMILES: c1ccc(N(c2ccc(-c3ccc4oc5ccccc5c4c3)cc2)c2ccc(-c3ccc4c5ccc6ccccc6c5n(-c5ccccc5)c4c3)cc2)cc1 ; c1ccc(-c2ccc(N(c3ccc(-c4ccc5sc6ccccc6c5c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5sc6ccccc6c5c4)cc3)c3ccc(-c4ccc5c6ccc7ccccc7c6n(-c6ccccc6)c5c4)cc3)c2)cc1]

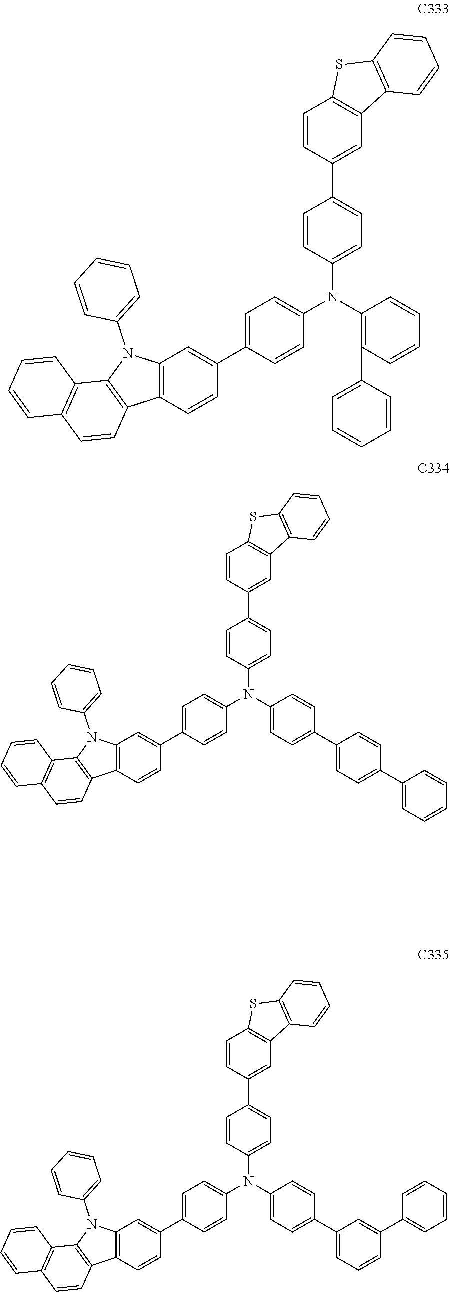 [SMILES: c1ccc(-c2ccccc2N(c2ccc(-c3ccc4sc5ccccc5c4c3)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6sc7ccccc7c6c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6sc7ccccc7c6c5)cc4)c4ccc(-c5ccc6c7ccc8ccccc8c7n(-c7ccccc7)c6c5)cc4)cc3)c2)cc1]

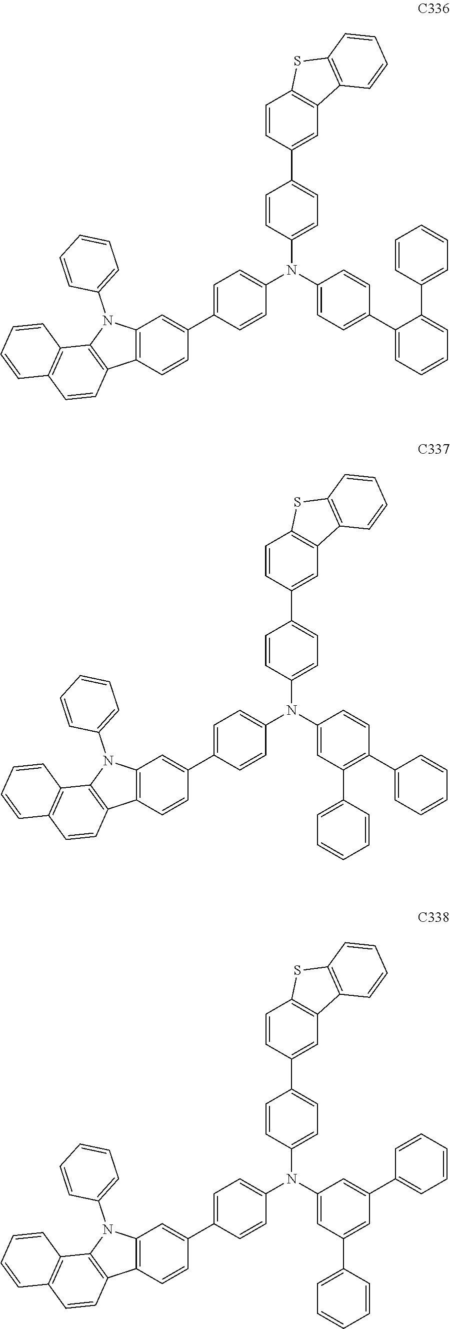 [SMILES: c1ccc(-c2ccccc2-c2ccc(N(c3ccc(-c4ccc5sc6ccccc6c5c4)cc3)c3ccc(-c4ccc5c6ccc7ccccc7c6n(-c6ccccc6)c5c4)cc3)cc2)cc1 ; c1ccc(-c2ccc(N(c3ccc(-c4ccc5sc6ccccc6c5c4)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5sc6ccccc6c5c4)cc3)c3ccc(-c4ccc5c6ccc7ccccc7c6n(-c6ccccc6)c5c4)cc3)c2)cc1]

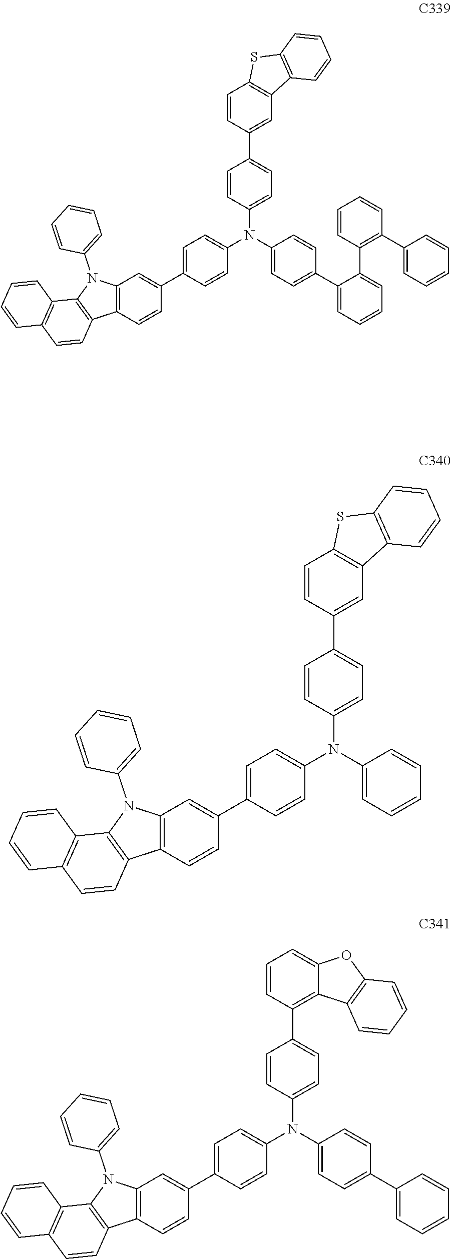 [SMILES: c1ccc(-c2ccccc2-c2ccccc2-c2ccc(N(c3ccc(-c4ccc5sc6ccccc6c5c4)cc3)c3ccc(-c4ccc5c6ccc7ccccc7c6n(-c6ccccc6)c5c4)cc3)cc2)cc1 ; c1ccc(N(c2ccc(-c3ccc4sc5ccccc5c4c3)cc2)c2ccc(-c3ccc4c5ccc6ccccc6c5n(-c5ccccc5)c4c3)cc2)cc1 ; c1ccc(-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1]

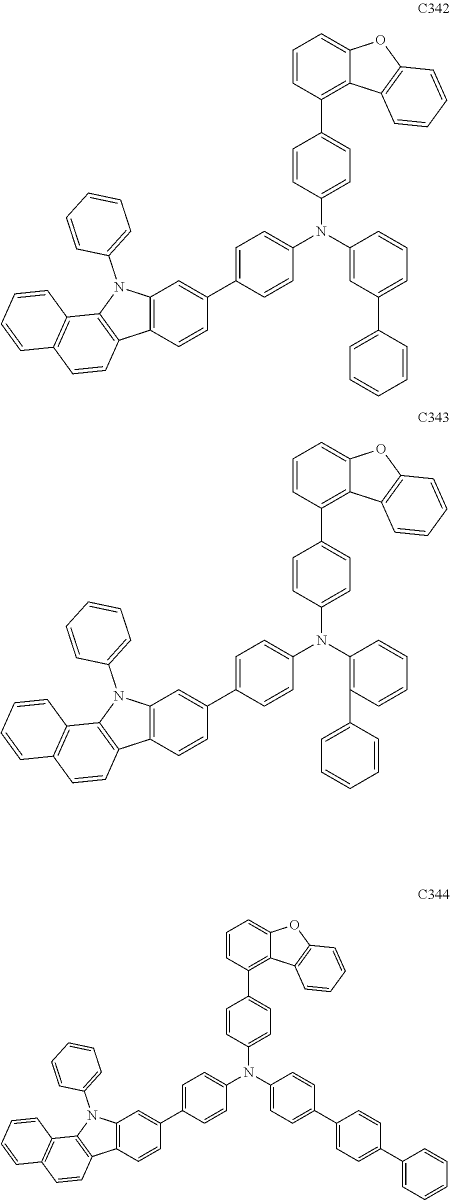 [SMILES: c1ccc(-c2cccc(N(c3ccc(-c4ccc5c6ccc7ccccc7c6n(-c6ccccc6)c5c4)cc3)c3ccc(-c4cccc5oc6ccccc6c45)cc3)c2)cc1 ; c1ccc(-c2ccccc2N(c2ccc(-c3ccc4c5ccc6ccccc6c5n(-c5ccccc5)c4c3)cc2)c2ccc(-c3cccc4oc5ccccc5c34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6oc7ccccc7c56)cc4)cc3)cc2)cc1]

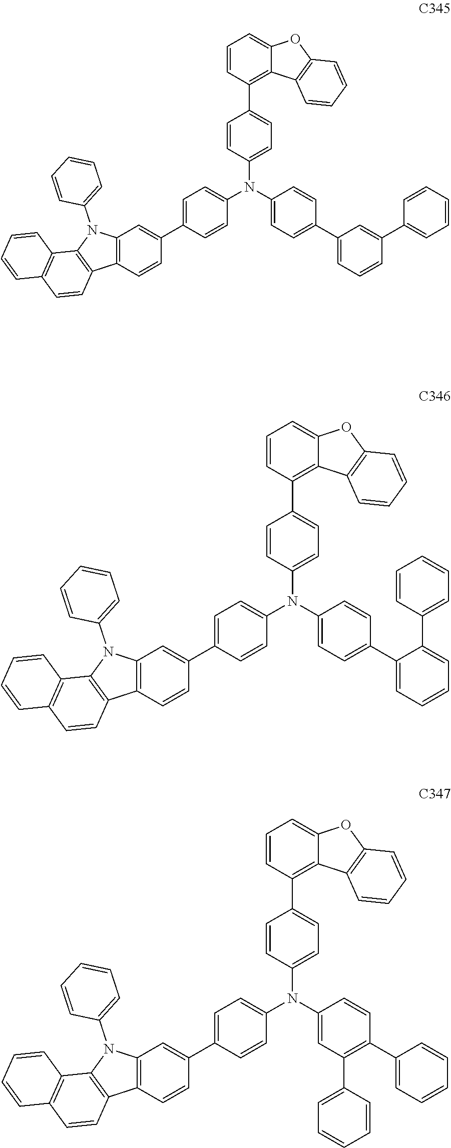 [SMILES: c1ccc(-c2cccc(-c3ccc(N(c4ccc(-c5ccc6c7ccc8ccccc8c7n(-c7ccccc7)c6c5)cc4)c4ccc(-c5cccc6oc7ccccc7c56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1 ; c1ccc(-c2ccc(N(c3ccc(-c4ccc5c6ccc7ccccc7c6n(-c6ccccc6)c5c4)cc3)c3ccc(-c4cccc5oc6ccccc6c45)cc3)cc2-c2ccccc2)cc1]

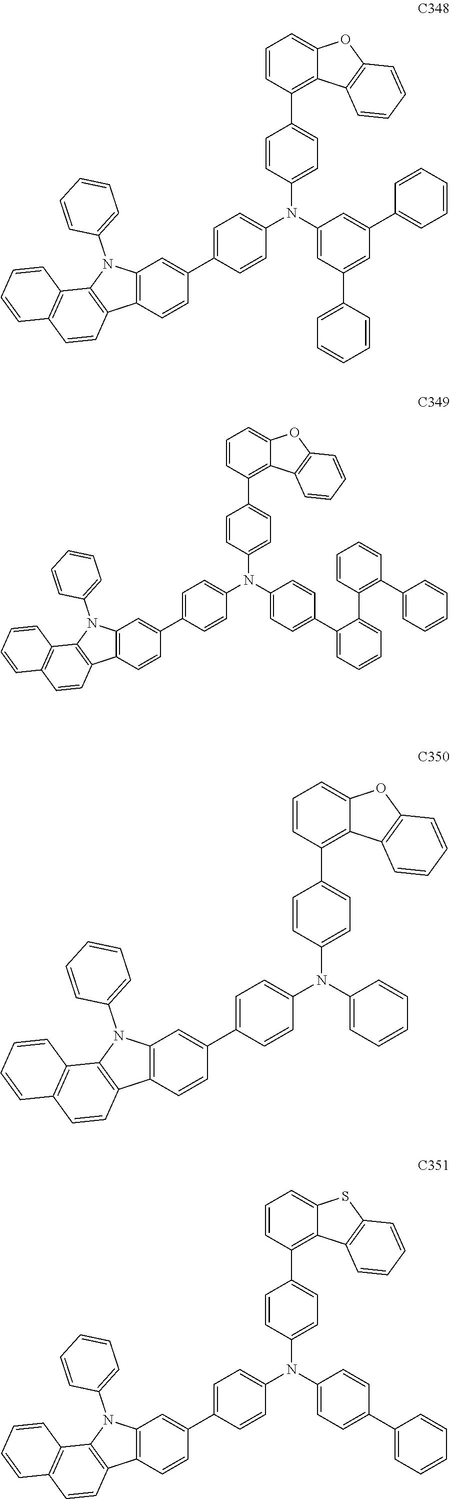 [SMILES: c1ccc(-c2cc(-c3ccccc3)cc(N(c3ccc(-c4ccc5c6ccc7ccccc7c6n(-c6ccccc6)c5c4)cc3)c3ccc(-c4cccc5oc6ccccc6c45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1 ; c1ccc(N(c2ccc(-c3ccc4c5ccc6ccccc6c5n(-c5ccccc5)c4c3)cc2)c2ccc(-c3cccc4oc5ccccc5c34)cc2)cc1 ; c1ccc(-c2ccc(N(c3ccc(-c4ccc5c6ccc7ccccc7c6n(-c6ccccc6)c5c4)cc3)c3ccc(-c4cccc5sc6ccccc6c45)cc3)cc2)cc1]

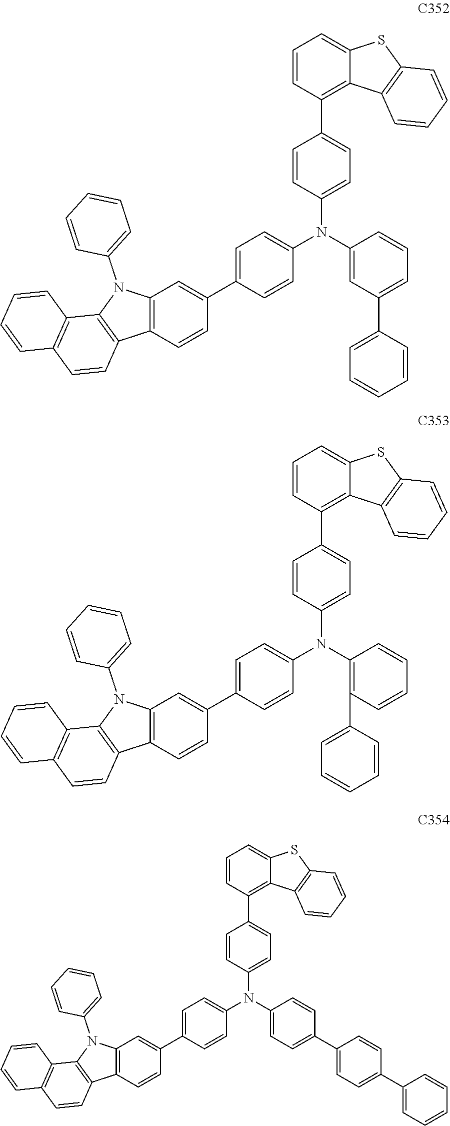 [SMILES: c1ccc(-c2cccc(N(c3ccc(-c4ccc5c6ccc7ccccc7c6n(-c6ccccc6)c5c4)cc3)c3ccc(-c4cccc5sc6ccccc6c45)cc3)c2)cc1 ; c1ccc(-c2ccccc2N(c2ccc(-c3ccc4c5ccc6ccccc6c5n(-c5ccccc5)c4c3)cc2)c2ccc(-c3cccc4sc5ccccc5c34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6sc7ccccc7c56)cc4)cc3)cc2)cc1]

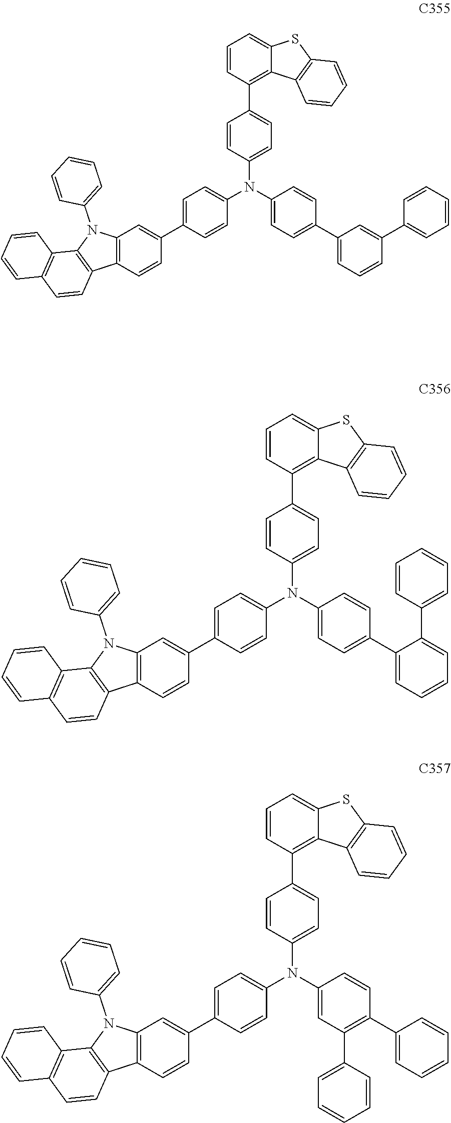 [SMILES: c1ccc(-c2cccc(-c3ccc(N(c4ccc(-c5ccc6c7ccc8ccccc8c7n(-c7ccccc7)c6c5)cc4)c4ccc(-c5cccc6sc7ccccc7c56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5sc6ccccc6c45)cc3)cc2)cc1 ; c1ccc(-c2ccc(N(c3ccc(-c4ccc5c6ccc7ccccc7c6n(-c6ccccc6)c5c4)cc3)c3ccc(-c4cccc5sc6ccccc6c45)cc3)cc2-c2ccccc2)cc1]

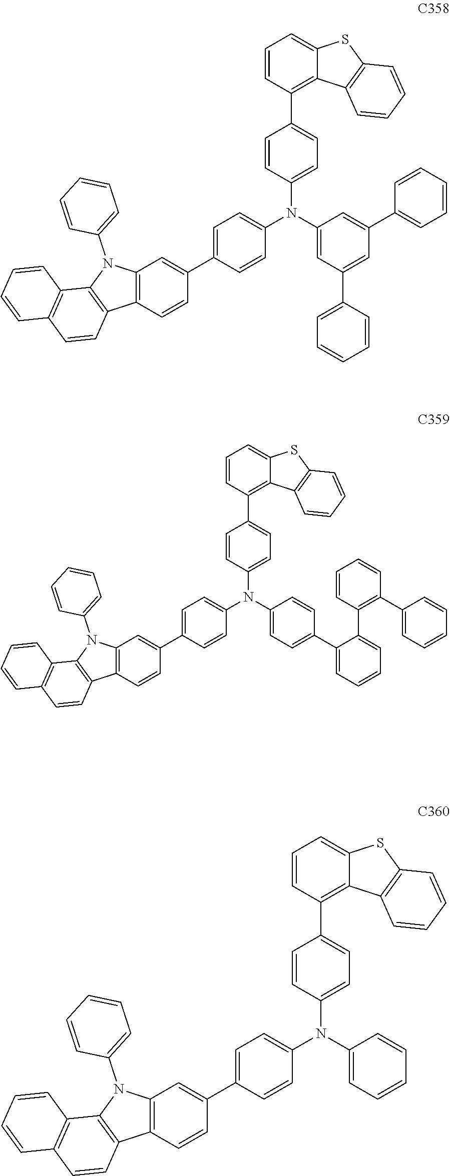 [SMILES: c1ccc(-c2cc(-c3ccccc3)cc(N(c3ccc(-c4ccc5c6ccc7ccccc7c6n(-c6ccccc6)c5c4)cc3)c3ccc(-c4cccc5sc6ccccc6c45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5sc6ccccc6c45)cc3)cc2)cc1 ; c1ccc(N(c2ccc(-c3ccc4c5ccc6ccccc6c5n(-c5ccccc5)c4c3)cc2)c2ccc(-c3cccc4sc5ccccc5c34)cc2)cc1]

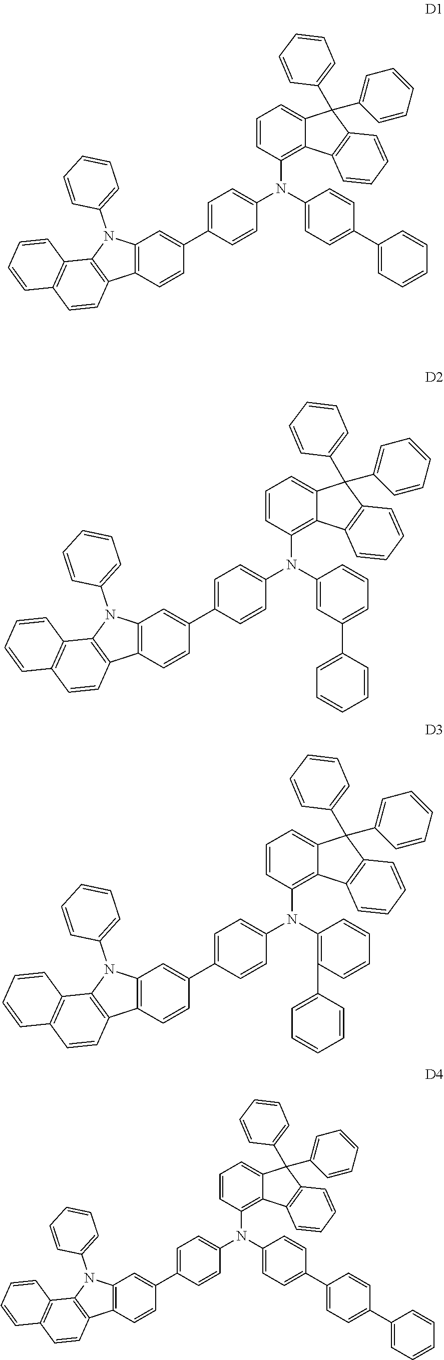 [SMILES: c1ccc(-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(-c2cccc(N(c3ccc(-c4ccc5c6ccc7ccccc7c6n(-c6ccccc6)c5c4)cc3)c3cccc4c3-c3ccccc3C4(c3ccccc3)c3ccccc3)c2)cc1 ; c1ccc(-c2ccccc2N(c2ccc(-c3ccc4c5ccc6ccccc6c5n(-c5ccccc5)c4c3)cc2)c2cccc3c2-c2ccccc2C3(c2ccccc2)c2ccccc2)cc1 ; c1ccc(-c2ccc(-c3ccc(N(c4ccc(-c5ccc6c7ccc8ccccc8c7n(-c7ccccc7)c6c5)cc4)c4cccc5c4-c4ccccc4C5(c4ccccc4)c4ccccc4)cc3)cc2)cc1]

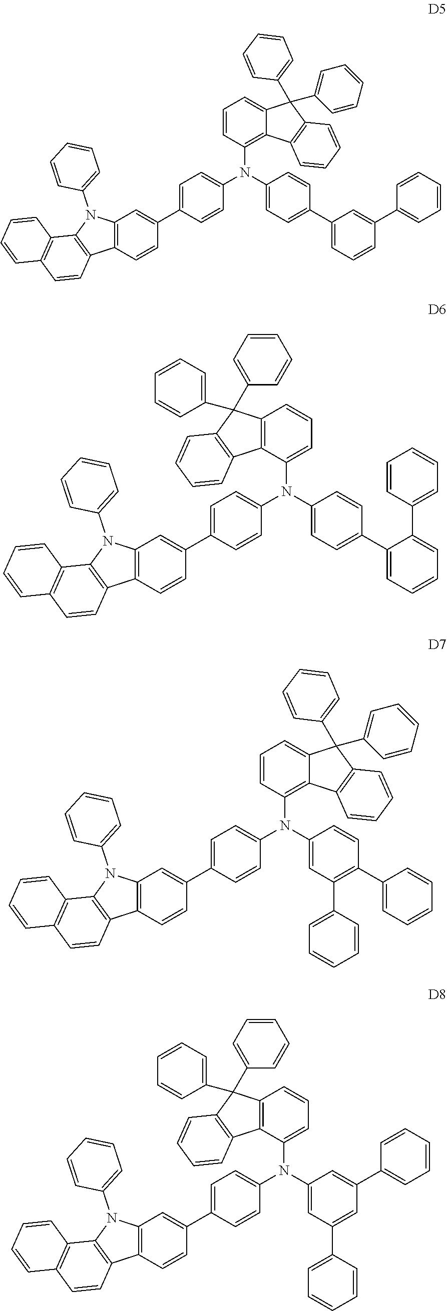 [SMILES: c1ccc(-c2cccc(-c3ccc(N(c4ccc(-c5ccc6c7ccc8ccccc8c7n(-c7ccccc7)c6c5)cc4)c4cccc5c4-c4ccccc4C5(c4ccccc4)c4ccccc4)cc3)c2)cc1 ; c1ccc(-c2ccccc2-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4c3-c3ccccc3C4(c3ccccc3)c3ccccc3)c2)cc1]

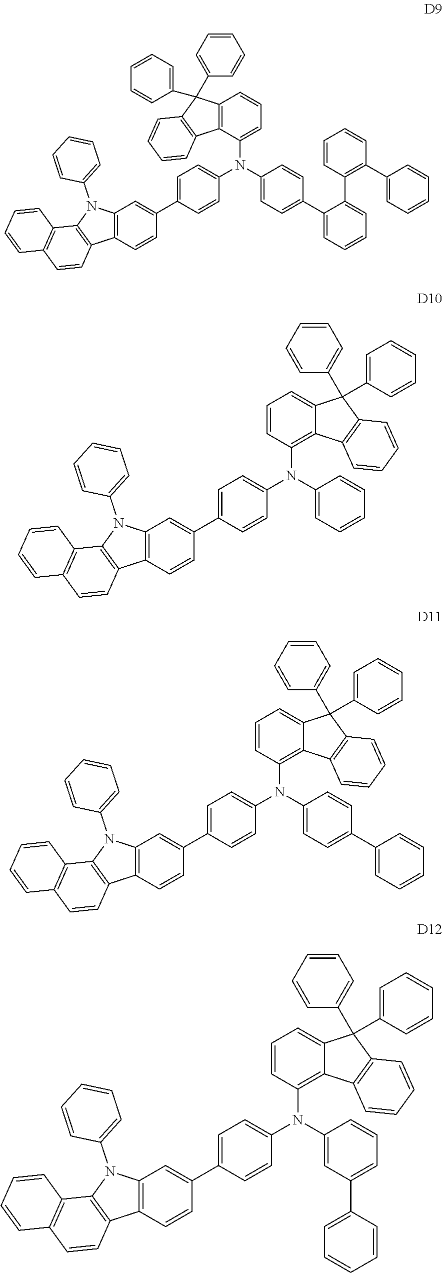 [SMILES: c1ccc(-c2ccccc2-c2ccccc2-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(N(c2ccc(-c3ccc4c5ccc6ccccc6c5n(-c5ccccc5)c4c3)cc2)c2cccc3c2-c2ccccc2C3(c2ccccc2)c2ccccc2)cc1 ; c1ccc(-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(-c2cccc(N(c3ccc(-c4ccc5c6ccc7ccccc7c6n(-c6ccccc6)c5c4)cc3)c3cccc4c3-c3ccccc3C4(c3ccccc3)c3ccccc3)c2)cc1]

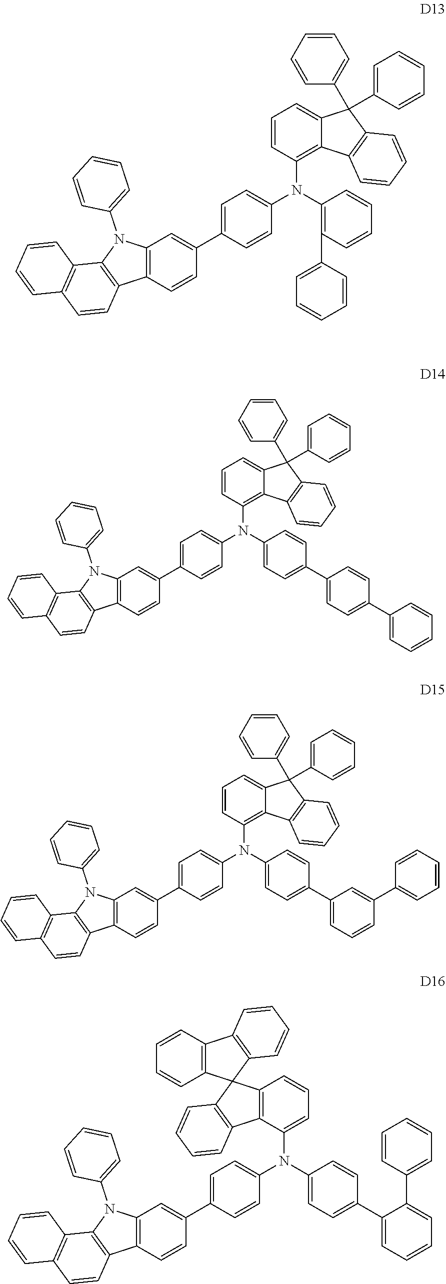 [SMILES: c1ccc(-c2ccccc2N(c2ccc(-c3ccc4c5ccc6ccccc6c5n(-c5ccccc5)c4c3)cc2)c2cccc3c2-c2ccccc2C3(c2ccccc2)c2ccccc2)cc1 ; c1ccc(-c2ccc(-c3ccc(N(c4ccc(-c5ccc6c7ccc8ccccc8c7n(-c7ccccc7)c6c5)cc4)c4cccc5c4-c4ccccc4C5(c4ccccc4)c4ccccc4)cc3)cc2)cc1 ; c1ccc(-c2cccc(-c3ccc(N(c4ccc(-c5ccc6c7ccc8ccccc8c7n(-c7ccccc7)c6c5)cc4)c4cccc5c4-c4ccccc4C5(c4ccccc4)c4ccccc4)cc3)c2)cc1 ; c1ccc(-c2ccccc2-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1]

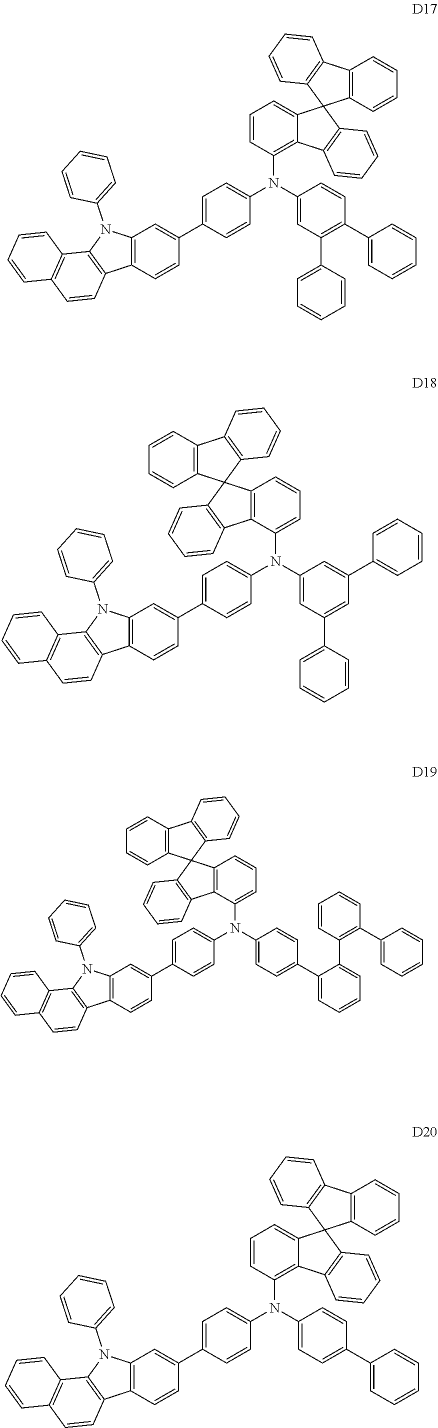 [SMILES: c1ccc(-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4c3-c3ccccc3C43c4ccccc4-c4ccccc43)c2)cc1 ; c1ccc(-c2ccccc2-c2ccccc2-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1 ; c1ccc(-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1]

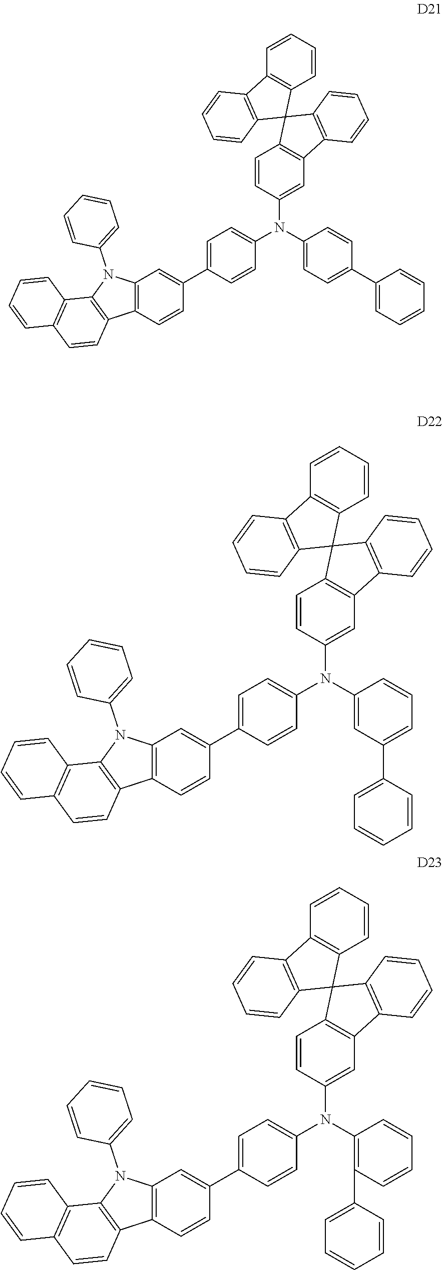 [SMILES: c1ccc(-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2)cc1 ; c1ccc(-c2cccc(N(c3ccc(-c4ccc5c6ccc7ccccc7c6n(-c6ccccc6)c5c4)cc3)c3ccc4c(c3)-c3ccccc3C43c4ccccc4-c4ccccc43)c2)cc1 ; c1ccc(-c2ccccc2N(c2ccc(-c3ccc4c5ccc6ccccc6c5n(-c5ccccc5)c4c3)cc2)c2ccc3c(c2)-c2ccccc2C32c3ccccc3-c3ccccc32)cc1]

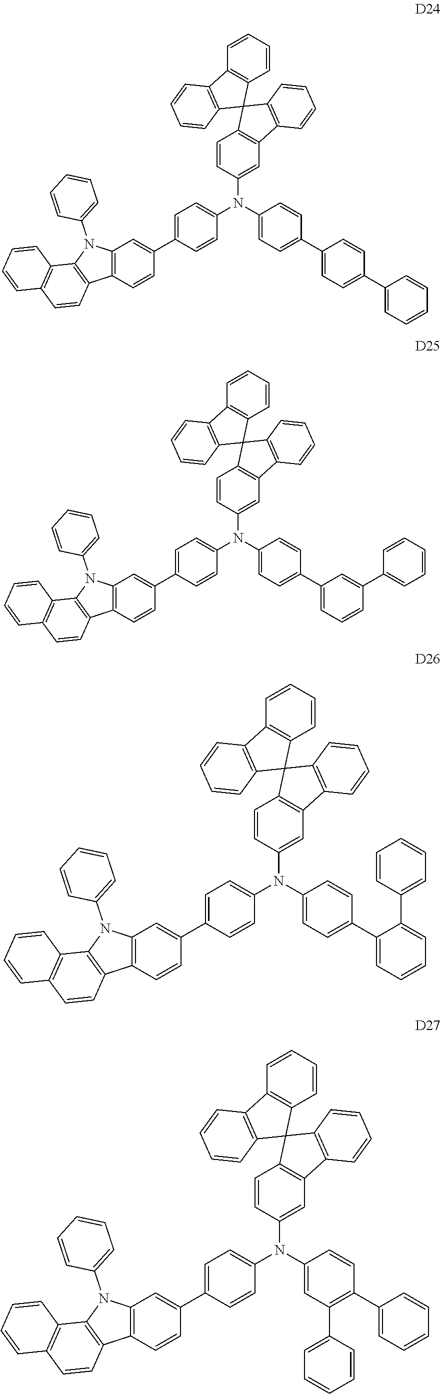 [SMILES: c1ccc(-c2ccc(-c3ccc(N(c4ccc(-c5ccc6c7ccc8ccccc8c7n(-c7ccccc7)c6c5)cc4)c4ccc5c(c4)-c4ccccc4C54c5ccccc5-c5ccccc54)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)-c4ccccc4C54c5ccccc5-c5ccccc54)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2)cc1 ; c1ccc(-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2-c2ccccc2)cc1]

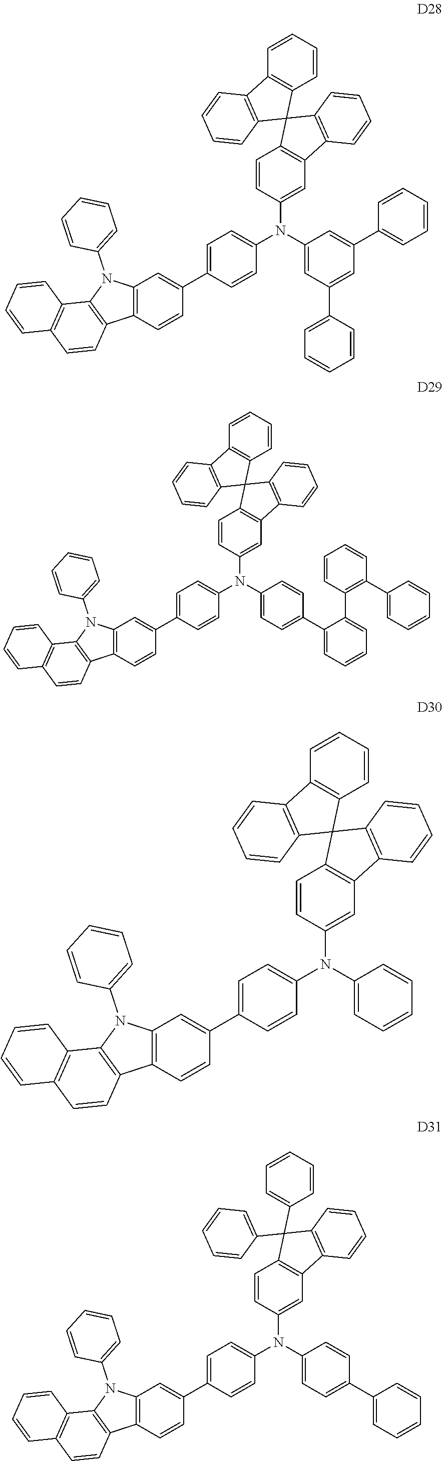 [SMILES: c1ccc(-c2cc(-c3ccccc3)cc(N(c3ccc(-c4ccc5c6ccc7ccccc7c6n(-c6ccccc6)c5c4)cc3)c3ccc4c(c3)-c3ccccc3C43c4ccccc4-c4ccccc43)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2)cc1 ; c1ccc(N(c2ccc(-c3ccc4c5ccc6ccccc6c5n(-c5ccccc5)c4c3)cc2)c2ccc3c(c2)-c2ccccc2C32c3ccccc3-c3ccccc32)cc1 ; c1ccc(-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2)cc1]

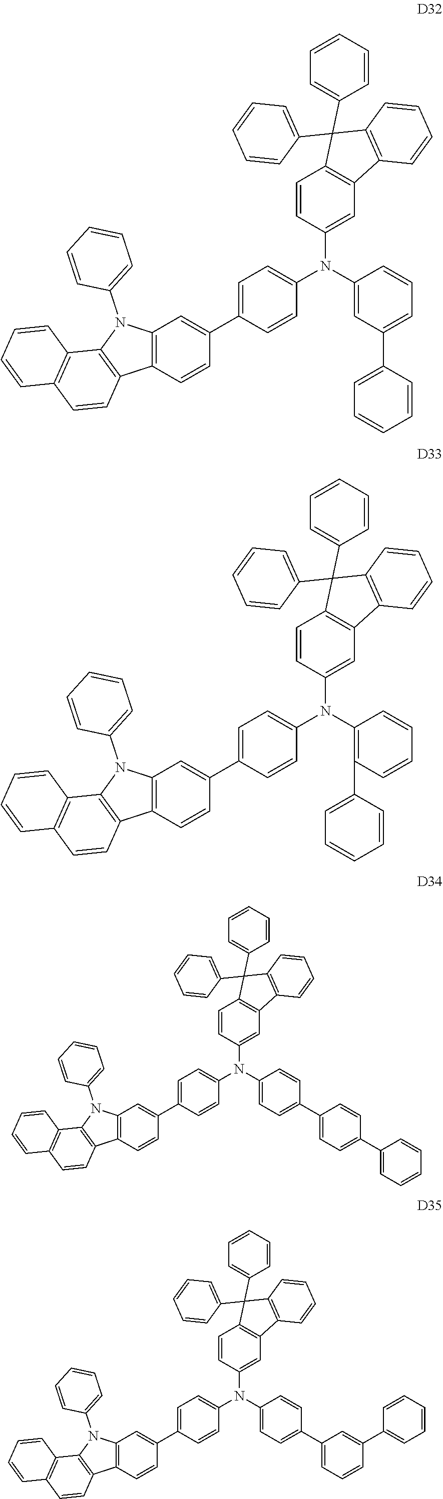 [SMILES: c1ccc(-c2cccc(N(c3ccc(-c4ccc5c6ccc7ccccc7c6n(-c6ccccc6)c5c4)cc3)c3ccc4c(c3)-c3ccccc3C4(c3ccccc3)c3ccccc3)c2)cc1 ; c1ccc(-c2ccccc2N(c2ccc(-c3ccc4c5ccc6ccccc6c5n(-c5ccccc5)c4c3)cc2)c2ccc3c(c2)-c2ccccc2C3(c2ccccc2)c2ccccc2)cc1 ; c1ccc(-c2ccc(-c3ccc(N(c4ccc(-c5ccc6c7ccc8ccccc8c7n(-c7ccccc7)c6c5)cc4)c4ccc5c(c4)-c4ccccc4C5(c4ccccc4)c4ccccc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)-c4ccccc4C5(c4ccccc4)c4ccccc4)cc3)c2)cc1]

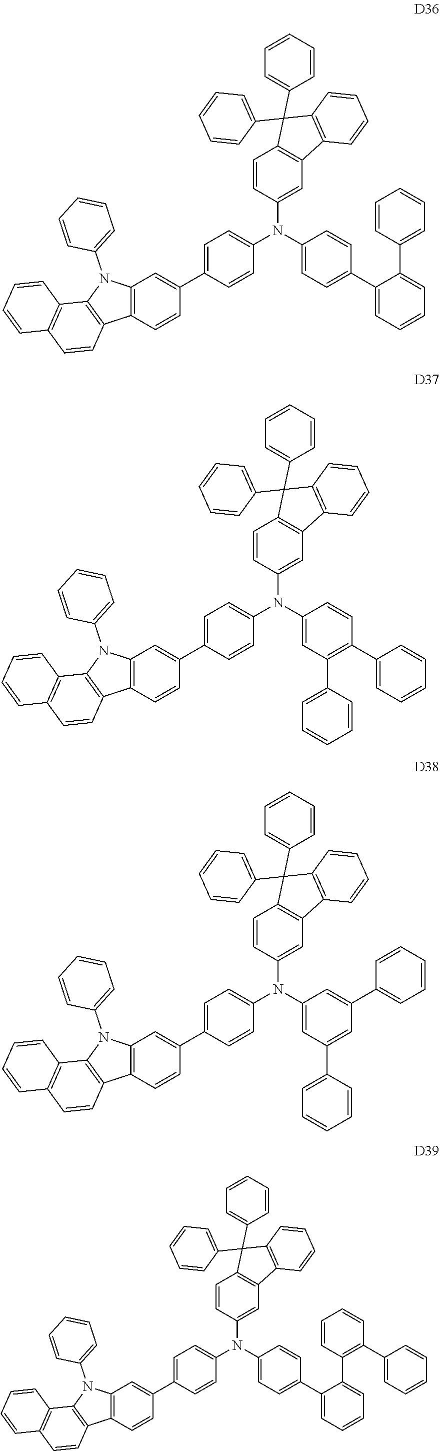 [SMILES: c1ccc(-c2ccccc2-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2)cc1 ; c1ccc(-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(c3)-c3ccccc3C4(c3ccccc3)c3ccccc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2)cc1]

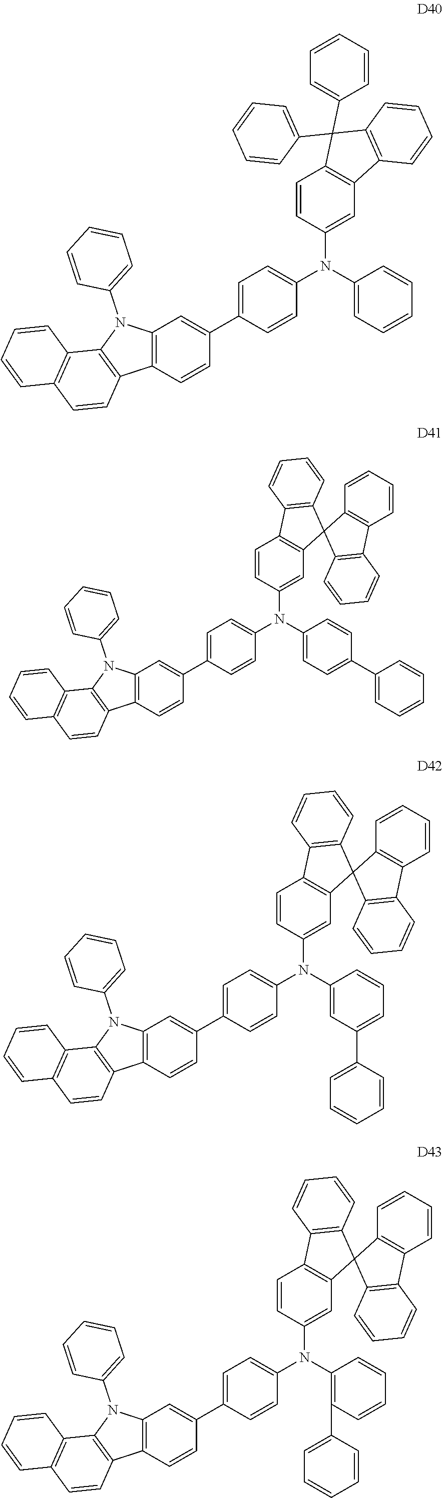 [SMILES: c1ccc(N(c2ccc(-c3ccc4c5ccc6ccccc6c5n(-c5ccccc5)c4c3)cc2)c2ccc3c(c2)-c2ccccc2C3(c2ccccc2)c2ccccc2)cc1 ; c1ccc(-c2ccc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)cc2)cc1 ; c1ccc(-c2cccc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)c2)cc1 ; c1ccc(-c2ccccc2N(c2ccc(-c3ccc4c5ccc6ccccc6c5n(-c5ccccc5)c4c3)cc2)c2ccc3c(c2)C2(c4ccccc4-c4ccccc42)c2ccccc2-3)cc1]

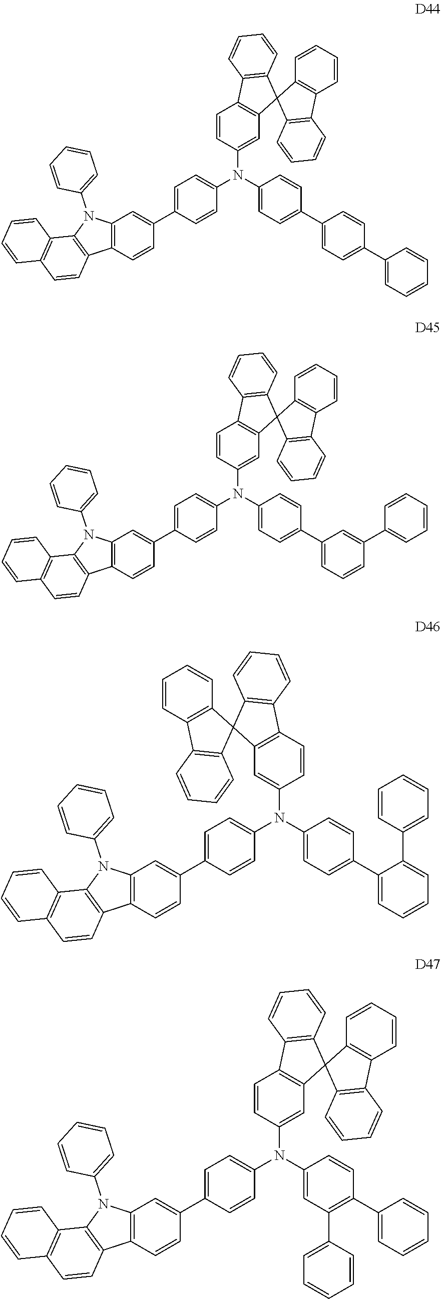

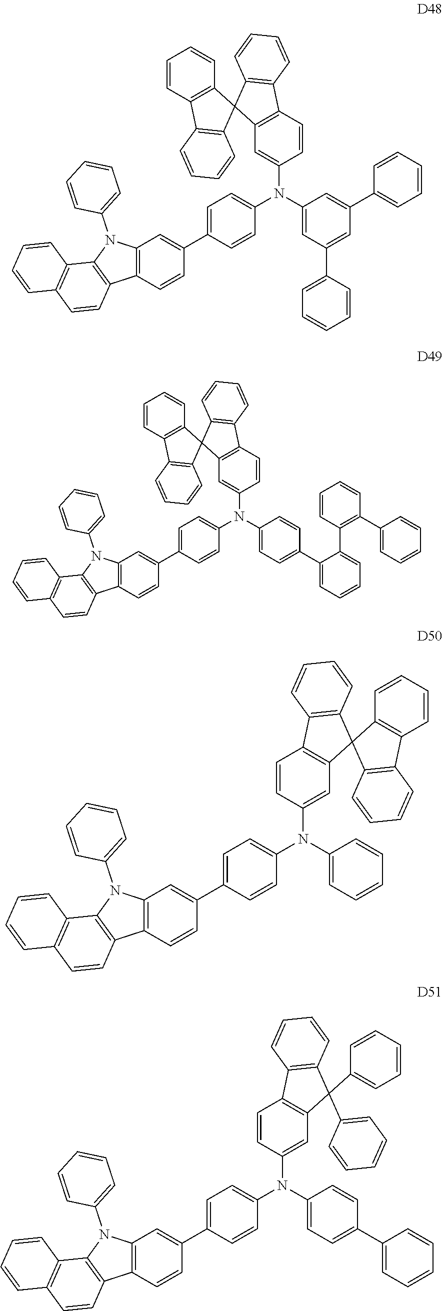 [SMILES: c1ccc(-c2cc(-c3ccccc3)cc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)cc2)cc1 ; c1ccc(N(c2ccc(-c3ccc4c5ccc6ccccc6c5n(-c5ccccc5)c4c3)cc2)c2ccc3c(c2)C2(c4ccccc4-c4ccccc42)c2ccccc2-3)cc1 ; c1ccc(-c2ccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)cc2)cc1]

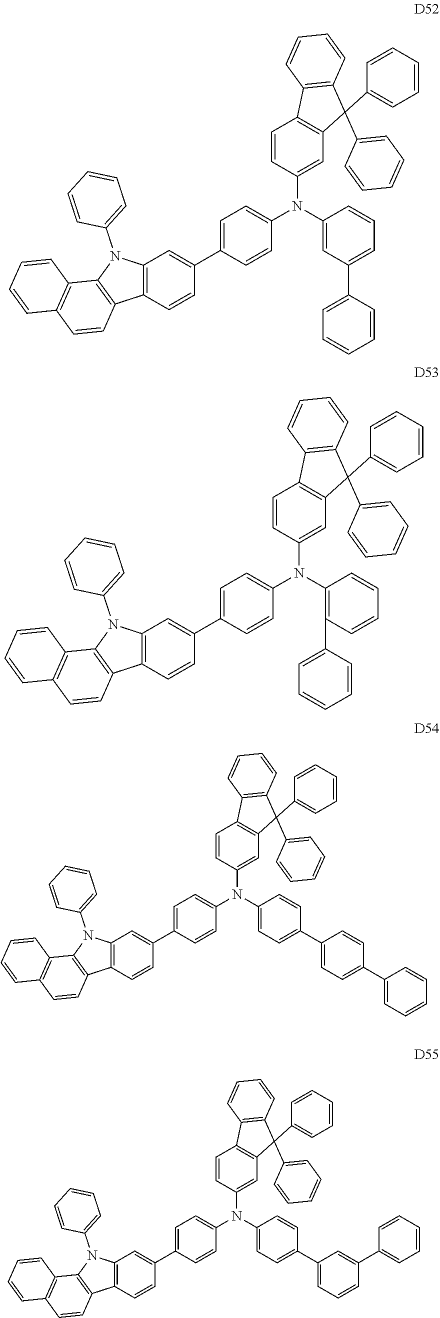 [SMILES: c1ccc(-c2cccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)c2)cc1 ; c1ccc(-c2ccccc2N(c2ccc(-c3ccc4c5ccc6ccccc6c5n(-c5ccccc5)c4c3)cc2)c2ccc3c(c2)C(c2ccccc2)(c2ccccc2)c2ccccc2-3)cc1 ; c1ccc(-c2ccc(-c3ccc(N(c4ccc(-c5ccc6c7ccc8ccccc8c7n(-c7ccccc7)c6c5)cc4)c4ccc5c(c4)C(c4ccccc4)(c4ccccc4)c4ccccc4-5)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)C(c4ccccc4)(c4ccccc4)c4ccccc4-5)cc3)c2)cc1]

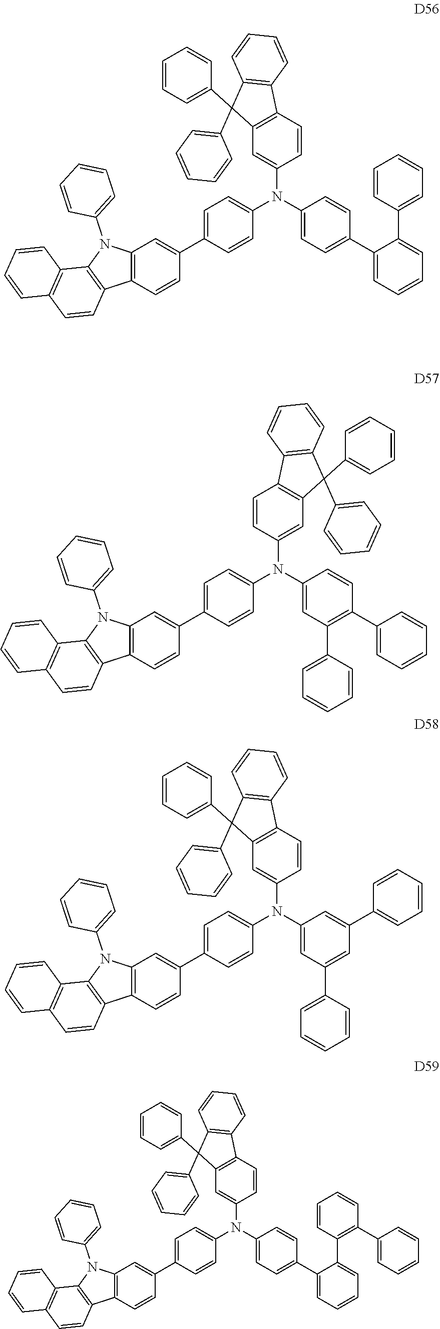

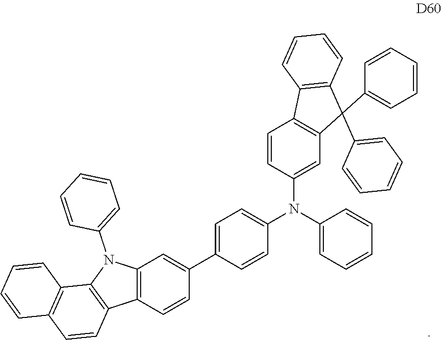 [SMILES: c1ccc(N(c2ccc(-c3ccc4c5ccc6ccccc6c5n(-c5ccccc5)c4c3)cc2)c2ccc3c(c2)C(c2ccccc2)(c2ccccc2)c2ccccc2-3)cc1]

13. An amine compound represented by Formula 1: 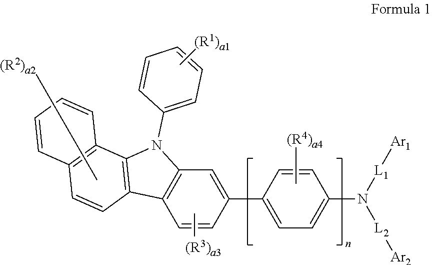
wherein, in Formula 1,
R¹ to R⁴ are each independently a hydrogen atom or a deuterium atom,
a1 is an integer from 0 to 5, a2 is an integer from 0 to 6, a3 is an integer from 0 to 3, a4 is an integer of 0 to 4, and n is 1 or 2,
L₁ and L₂ are each independently a direct linkage, a substituted or unsubstituted arylene group having 6 to 30 ring-forming carbon atoms, or a substituted or unsubstituted heteroarylene group having 5 to 30 ring-forming carbon atoms,
Ar₁ and Ar₂ are each independently a substituted or unsubstituted aryl group having 6 to 40 ring-forming carbon atoms, or a substituted or unsubstituted heteroaryl group having 5 to 40 ring-forming carbon atoms, or any one among Formula 2 to Formula 5, and
at least one of Ar₁ or Ar₂ is any one among Formula 2 to Formula 5: 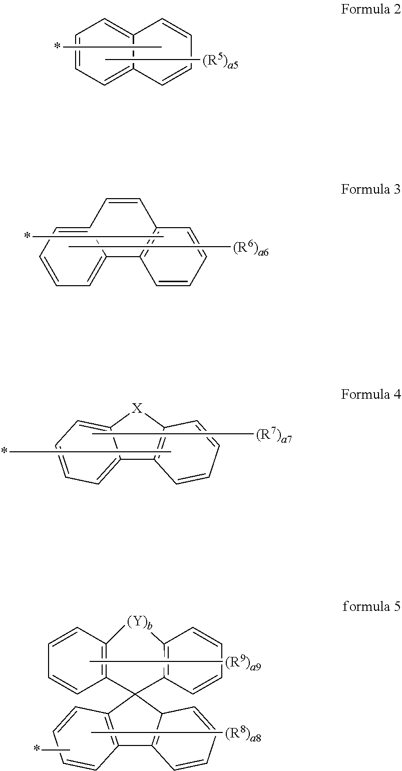
wherein, in Formula 2 to Formula 5, [symbol]
is a part bonded to L₁ or L₂ in Formula 1,
R⁵ to R⁹ are each independently a hydrogen atom, a deuterium atom, a halogen atom, a substituted or unsubstituted aryl group having 6 to 30 ring-forming carbon atoms, a substituted or unsubstituted heteroaryl group having 5 to 30 ring-forming carbon atoms, a substituted or unsubstituted alkyl group having 1 to 30 carbon atoms, or a substituted or unsubstituted alkenyl group having 2 to 30 carbon atoms, or are bonded to an adjacent group to form a ring,
a5, a7, and a8 are each independently an integer from 0 to 7, a6 is an integer from 0 to 9, and a9 is an integer from 0 to 8,
in Formula 4 above, X is O or S, and
in Formula 5 above, b is 0 or 1, and when b is 1, Y is a direct linkage.

14. The amine compound of claim 13, wherein Formula 1 is represented by Formula 1-1: 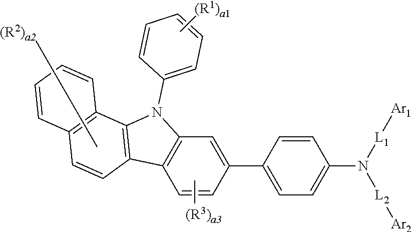
wherein, in Formula 1-1, R¹ to R³, a1 to a3, L₁, L₂, Ar₁, and Ar₂ are the same as defined in Formula 1.

15. The amine compound of claim 13, wherein L₁ and L₂ are each independently a direct linkage, or a substituted or unsubstituted arylene group having 6 to 30 ring-forming carbon atoms.

16. The amine compound of claim 13, wherein Formula 2 is represented by any one among 2-1 and 2-2: 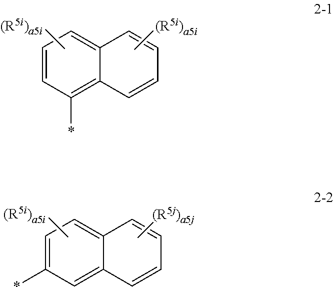
wherein, in 2-1 and 2-2,
R⁵ⁱ and R^{5j} are each independently a hydrogen atom, a deuterium atom, or a substituted or unsubstituted aryl group having 6 to 30 ring-forming carbon atoms, and
a5i is an integer from 0 to 3, and a5j is an integer from 0 to 4.

17. The amine compound of claim 13, wherein Formula 3 is represented by any one among 3-1 to 3-3: 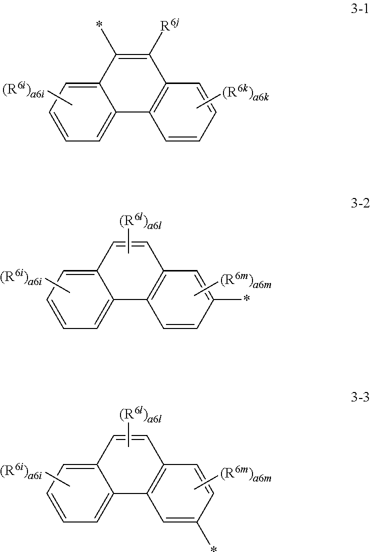
wherein, in 3-1 to 3-3,
R⁶ⁱ, R^{6j}, R^{6k}, R⁶, and R^{6m} are each independently a hydrogen atom, a deuterium atom, or a substituted or unsubstituted aryl group having 6 to 30 ring-forming carbon atoms,
a6i is an integer from 0 to 4,
in 3-1 above, a6k is an integer from 0 to 4, and
in 3-2 and 3-3, a61 is an integer from 0 to 2, and a6m is an integer from 0 to 3.

18. The amine compound of claim 1, wherein Formula 4 is represented by any one among 4-1 to 4-4: 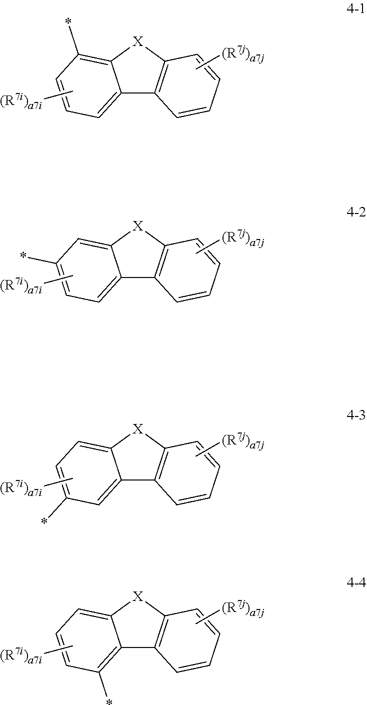
wherein, in 4-1 to 4-4,
R⁷ⁱ and R^{7j} are each independently a hydrogen atom, a deuterium atom, or a substituted or unsubstituted aryl group having 6 to 30 ring-forming carbon atoms, or are bonded to an adjacent group to form a ring, and
a7i is an integer from 0 to 3, and a7j is an integer from 0 to 4.

19. The amine compound of claim 13, wherein Formula 5 is represented by any one among 5-1 to 5-6: 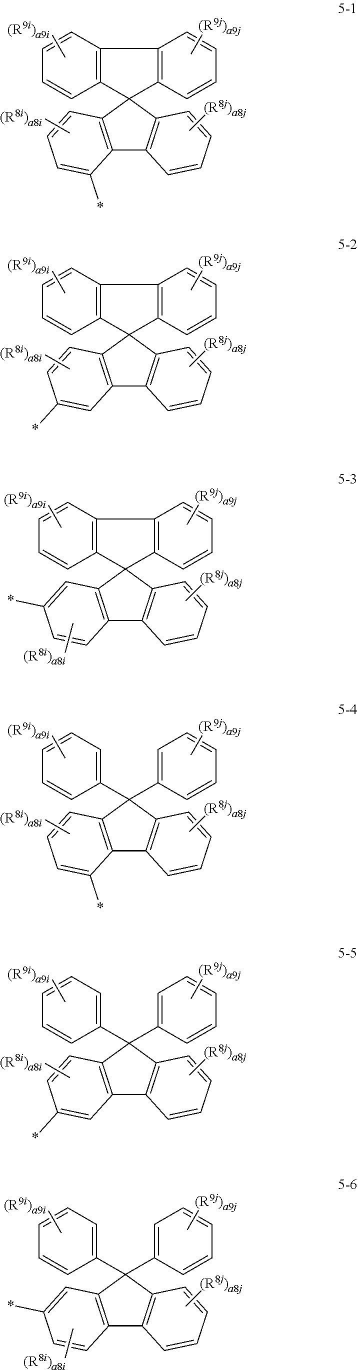
wherein, in 5-1 to 5-6,
R⁸ⁱ, R^{8j}, R⁹ⁱ, and R^{9j} are each independently a hydrogen atom, a deuterium atom, or a substituted or unsubstituted aryl group having 6 to 30 ring-forming carbon atoms, or are bonded to an adjacent group to form a ring,
a8i is an integer from 0 to 3, and a9i and a9i is an integer from 0 to 4.

20. The amine compound of claim 13, wherein Formula 1 is represented by any one among compounds of Compound Group 1: 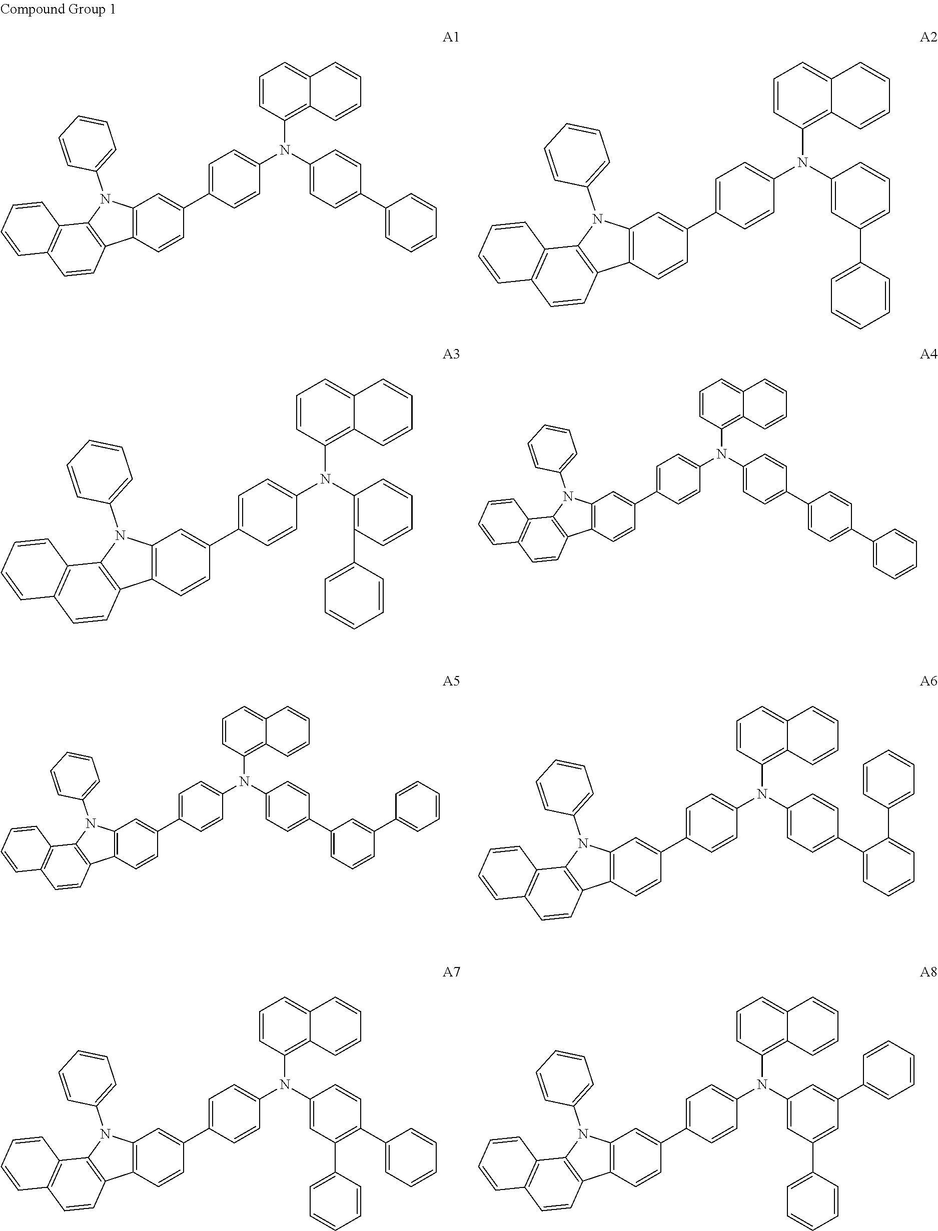 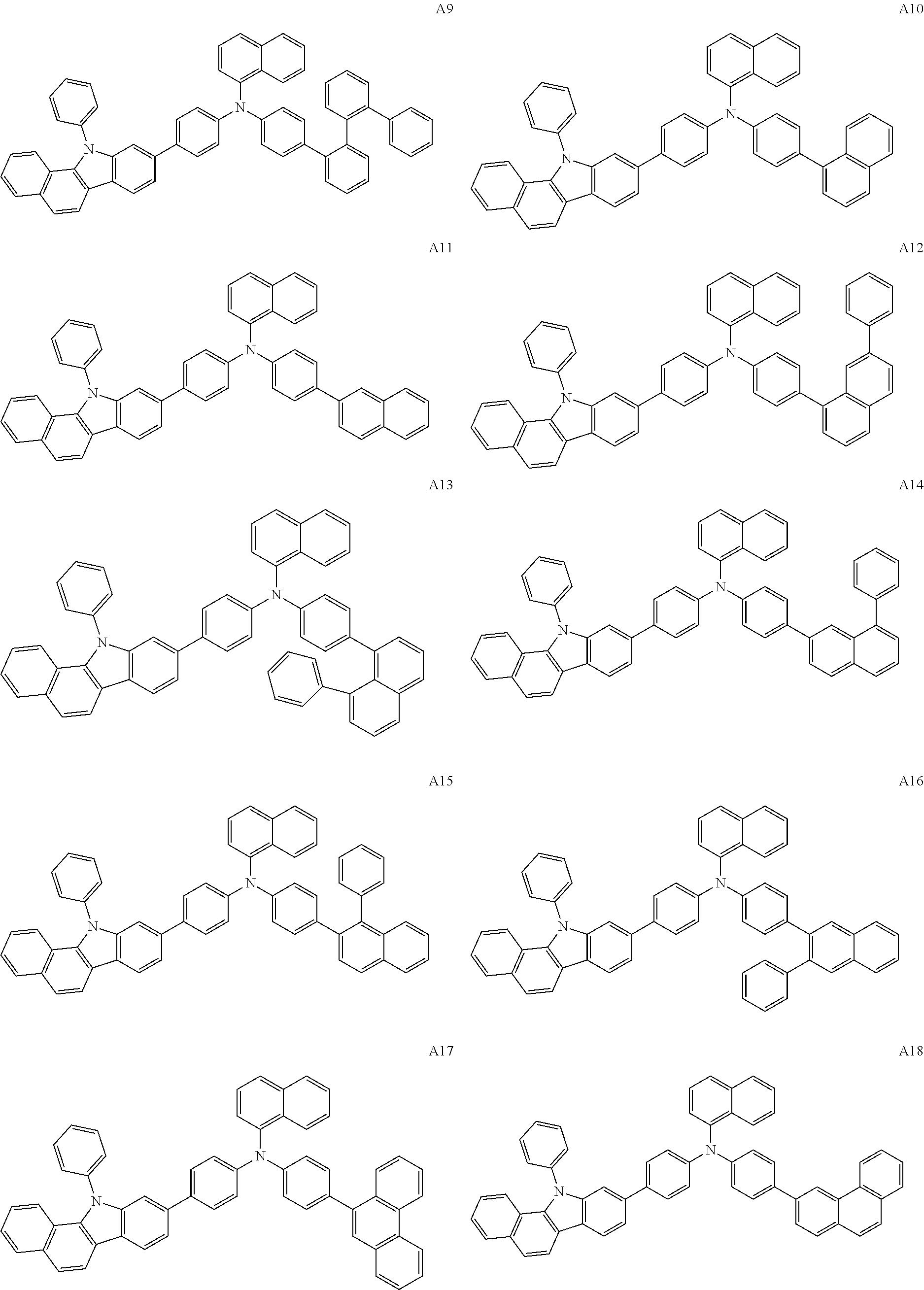 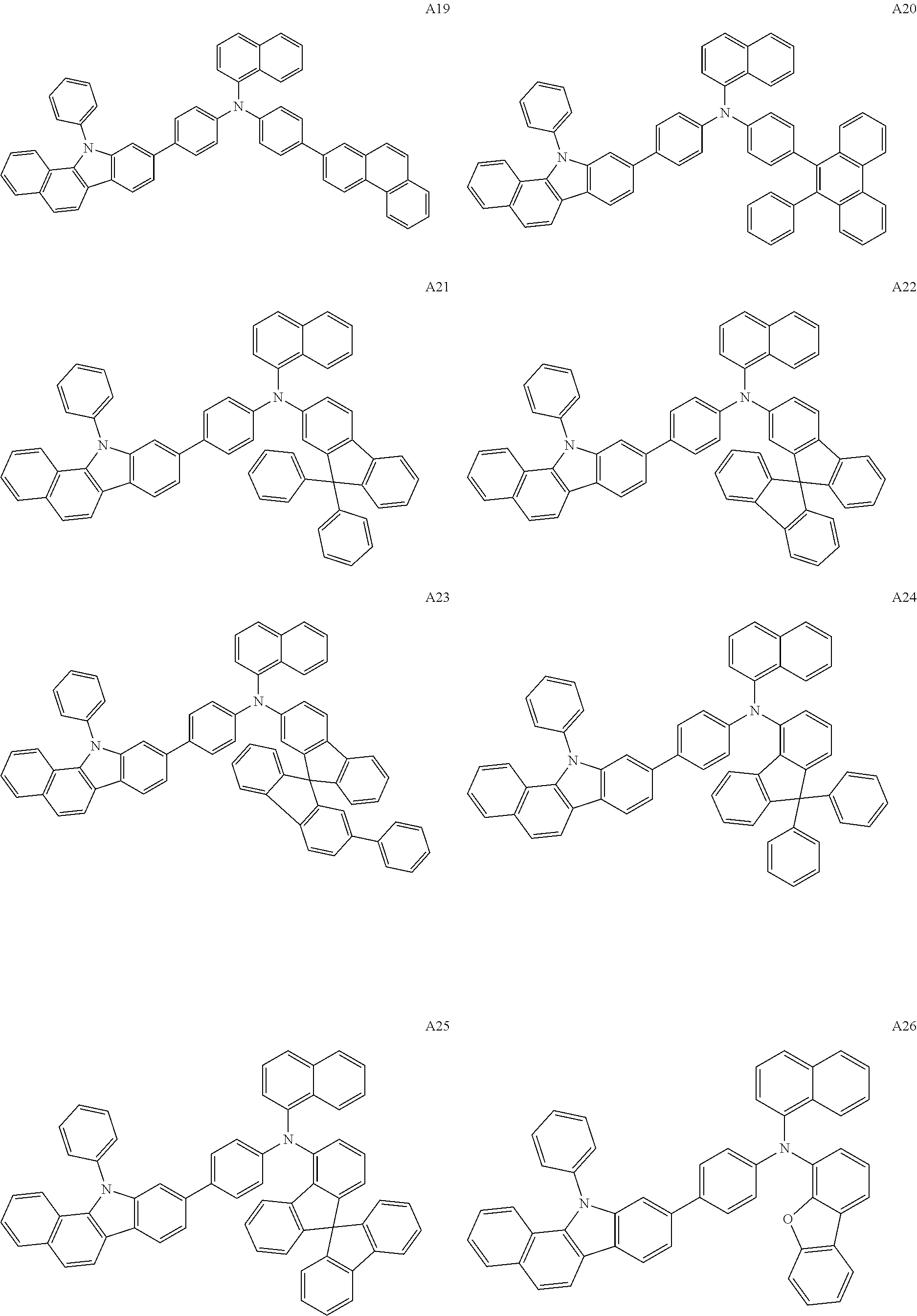 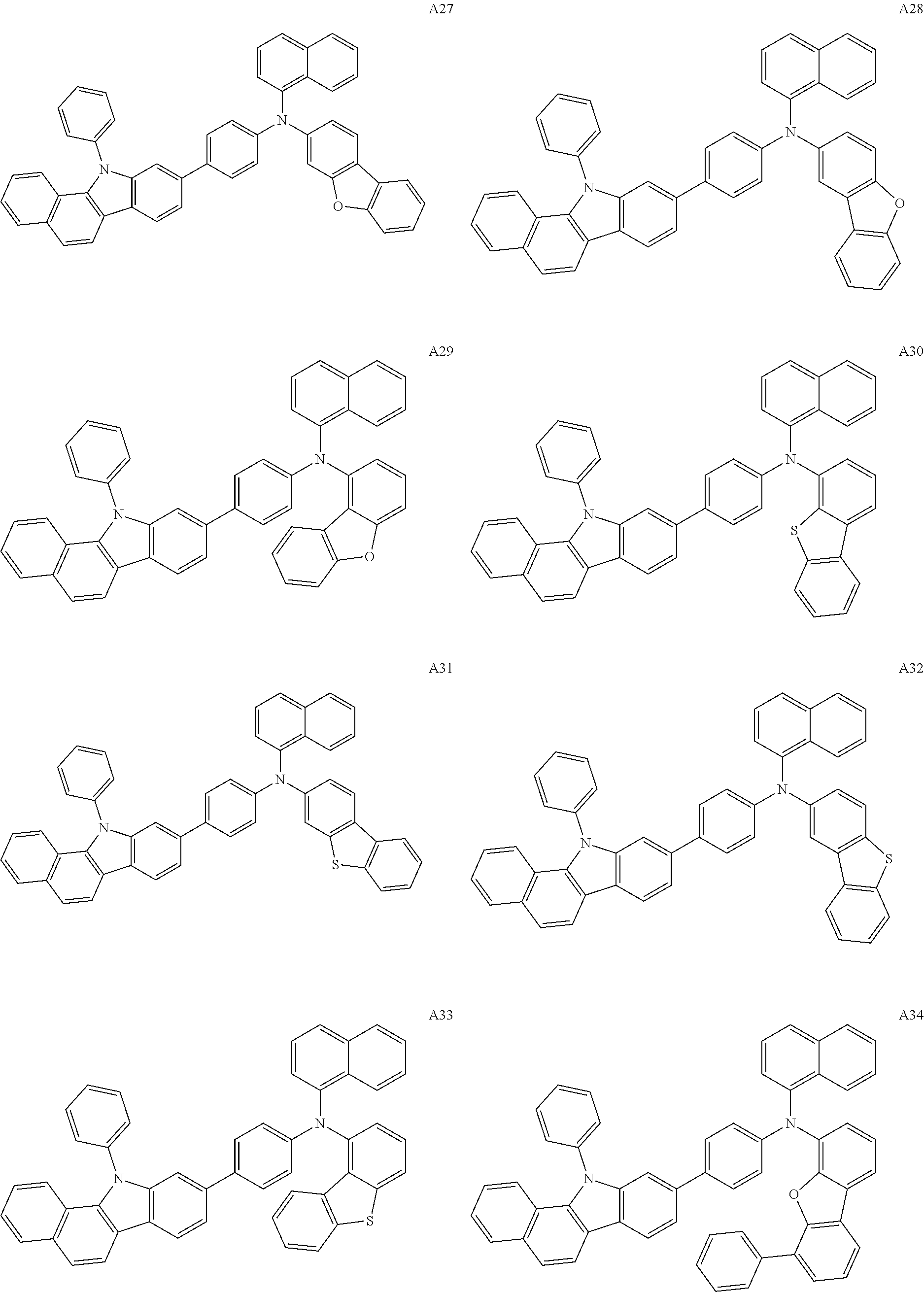 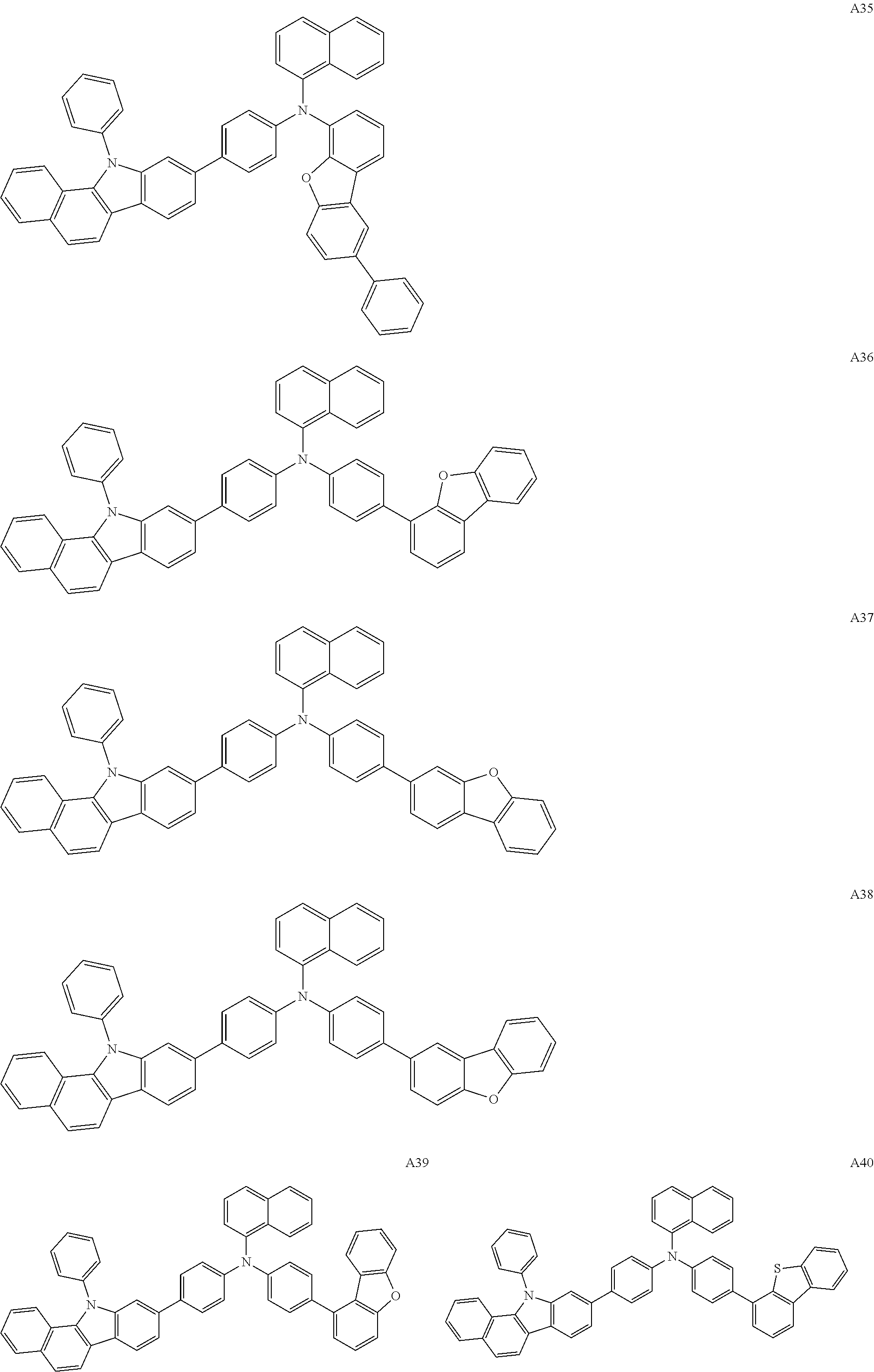 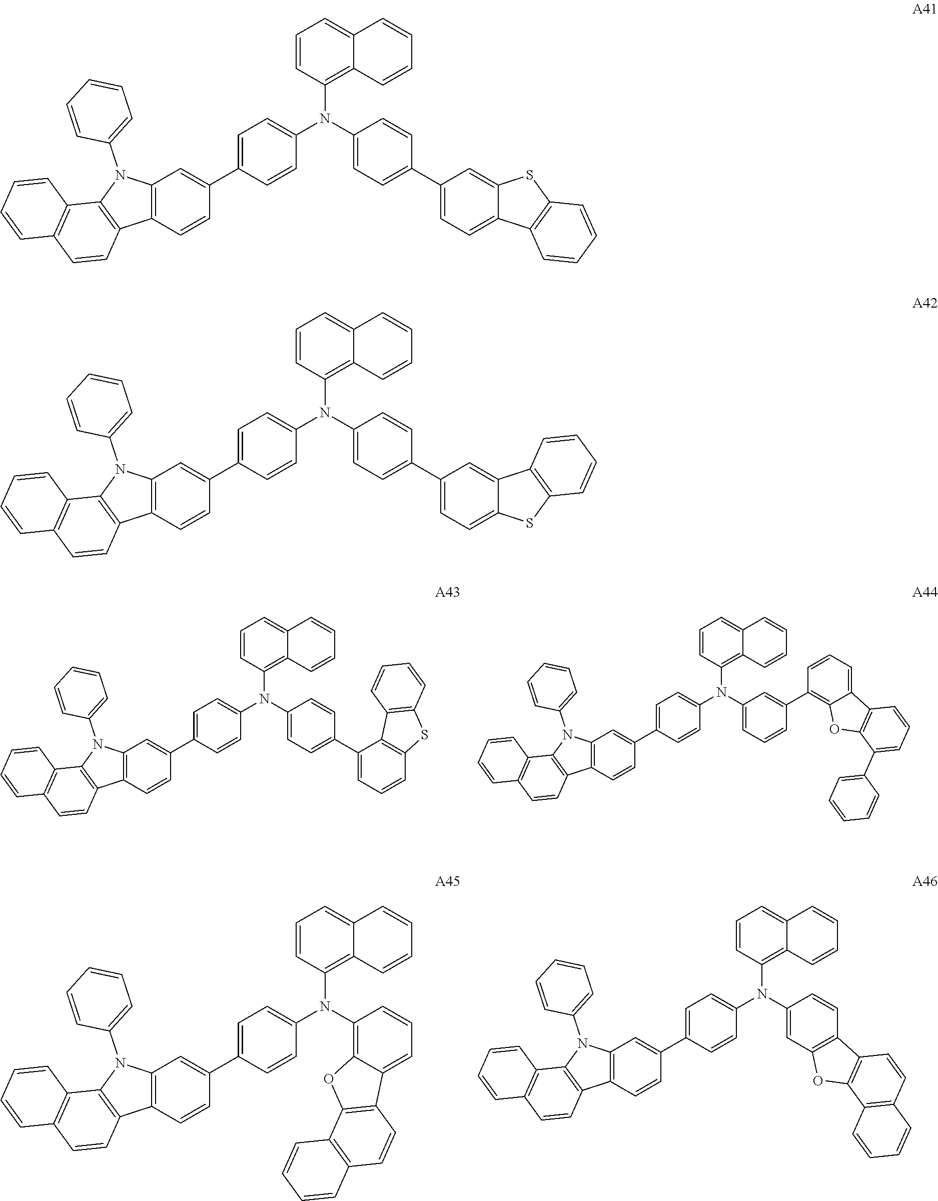 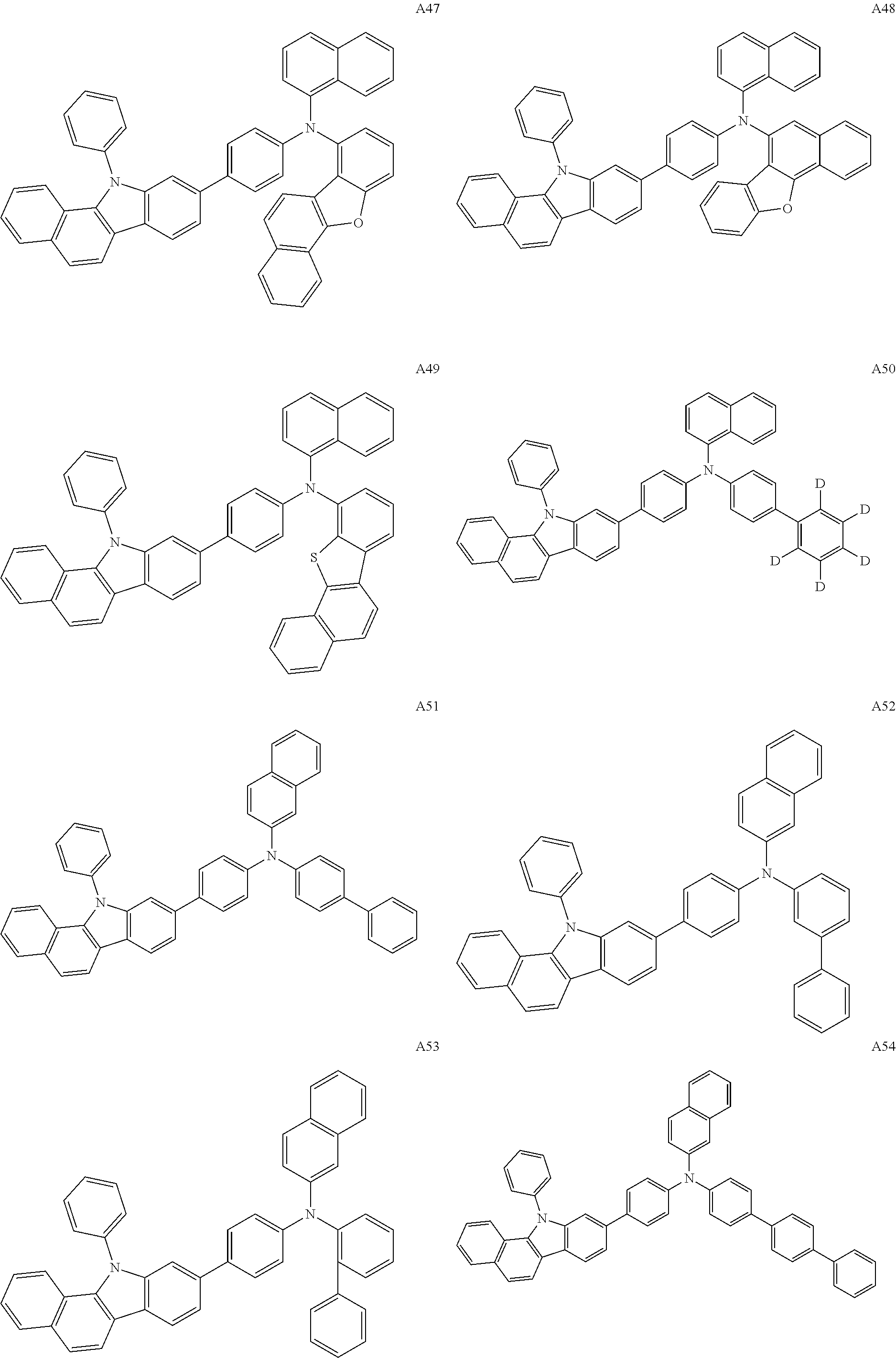 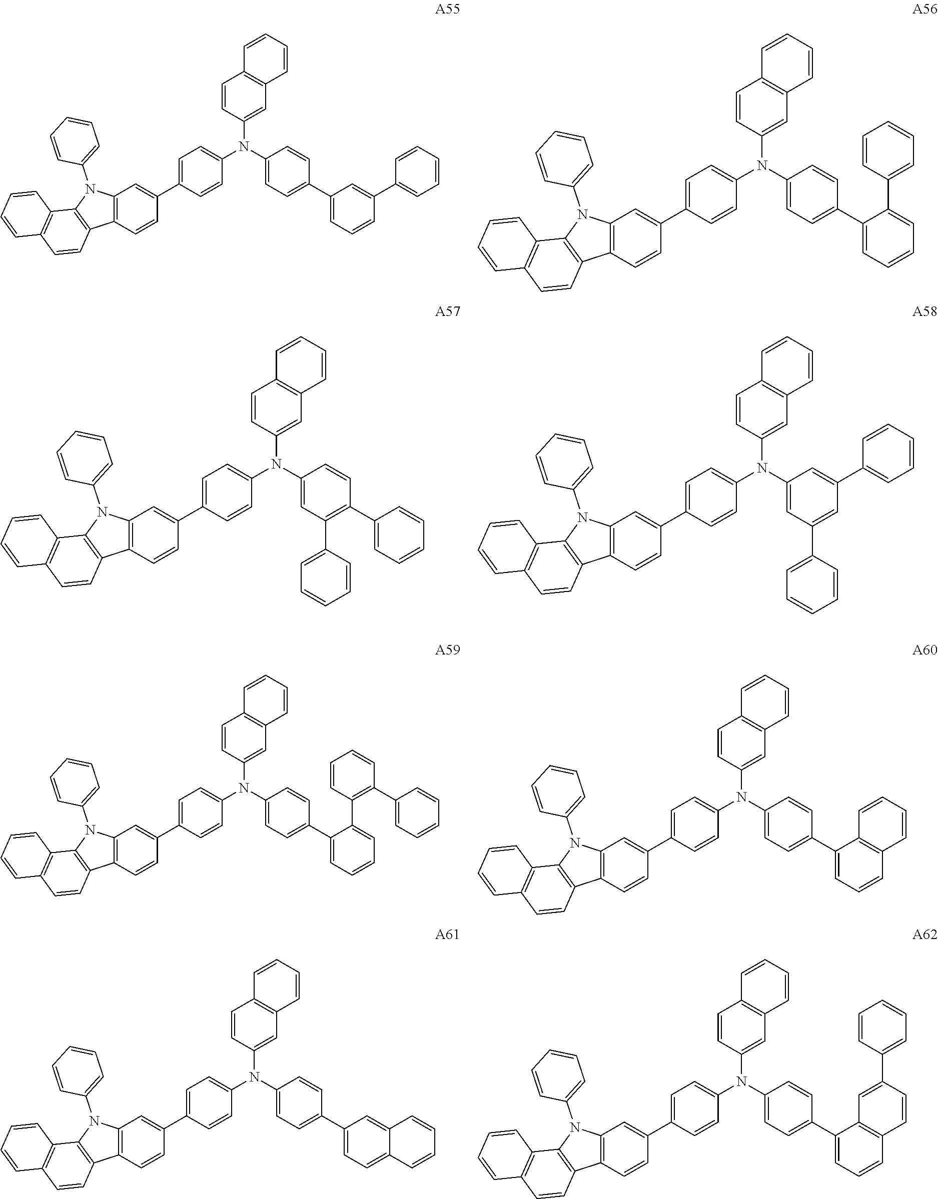 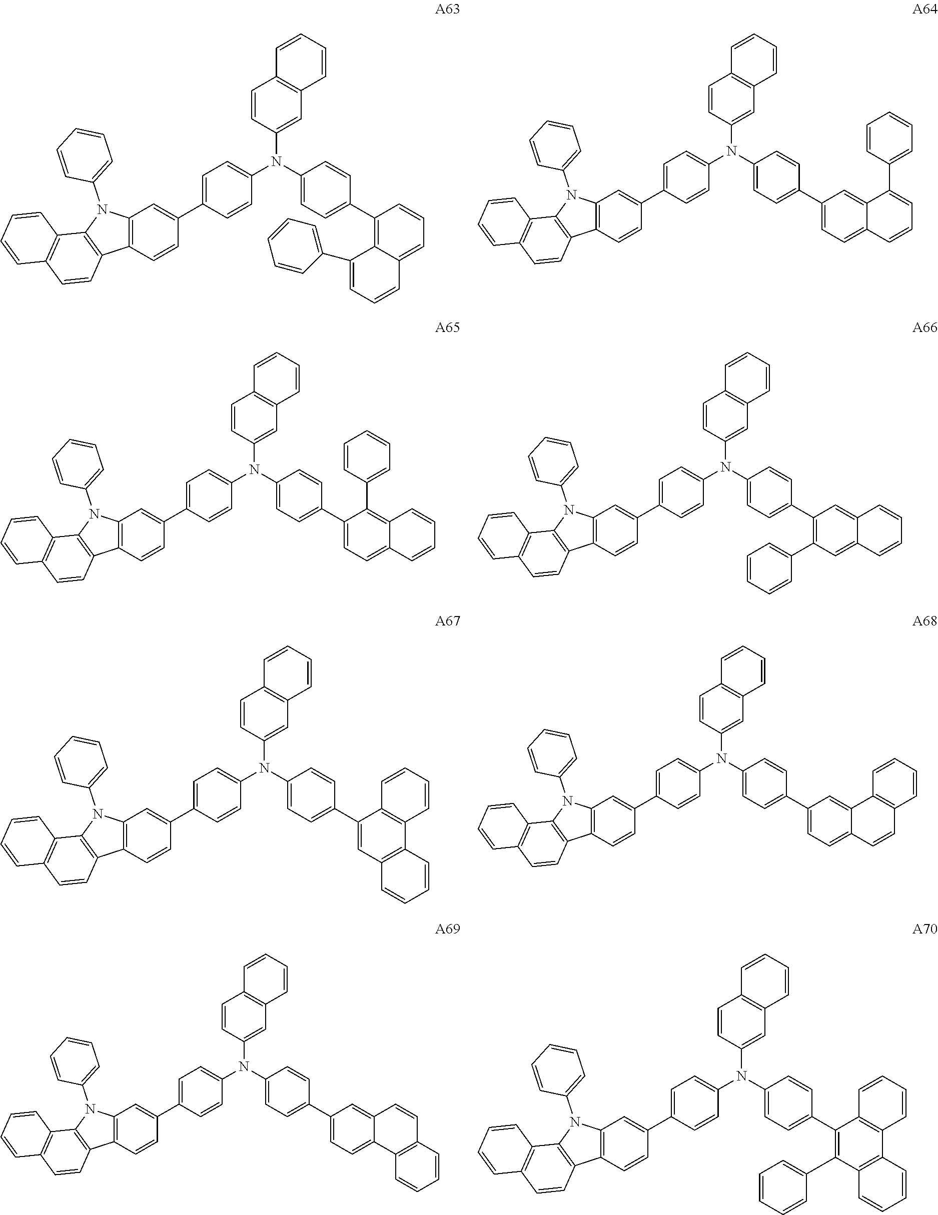 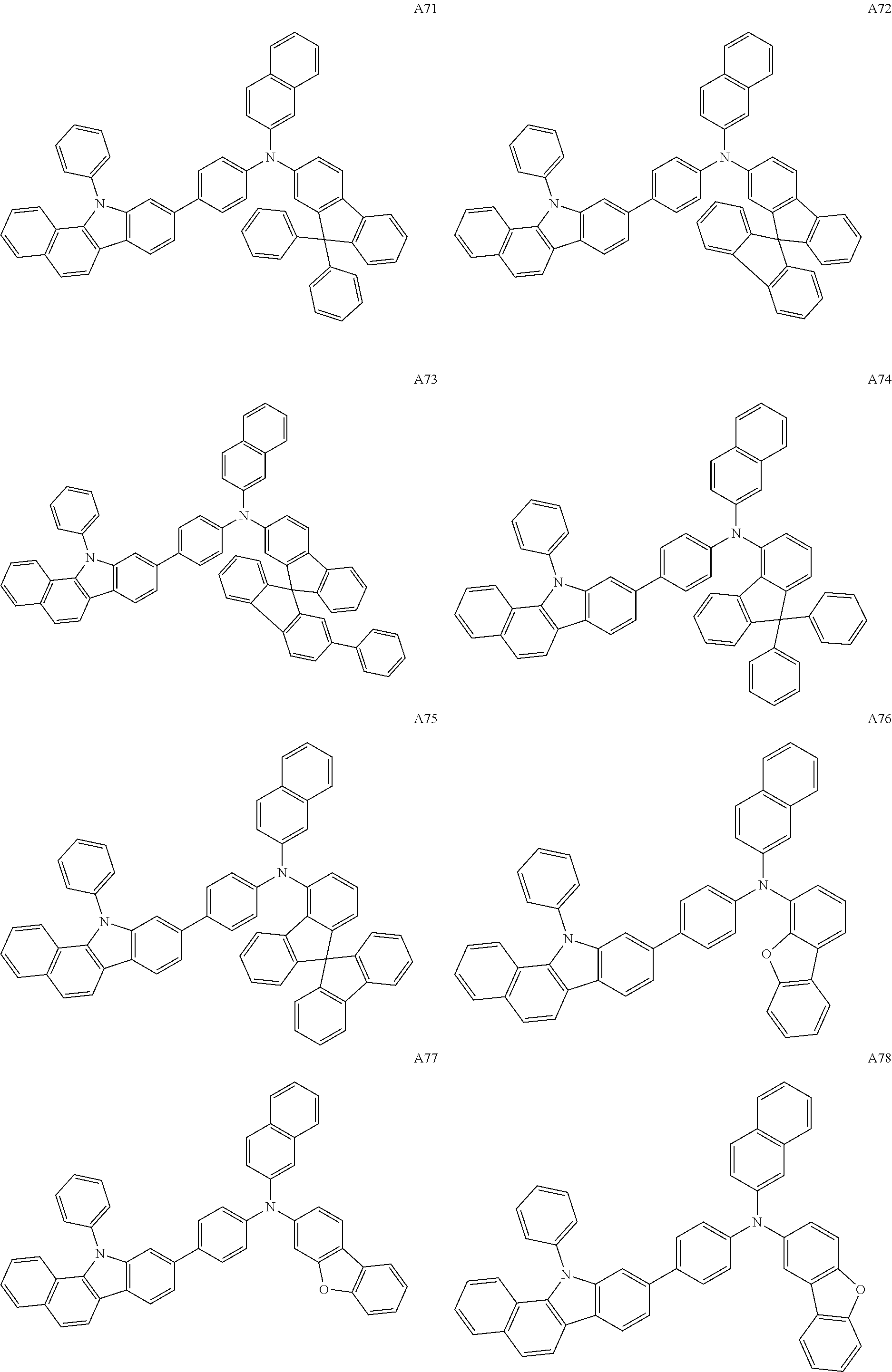 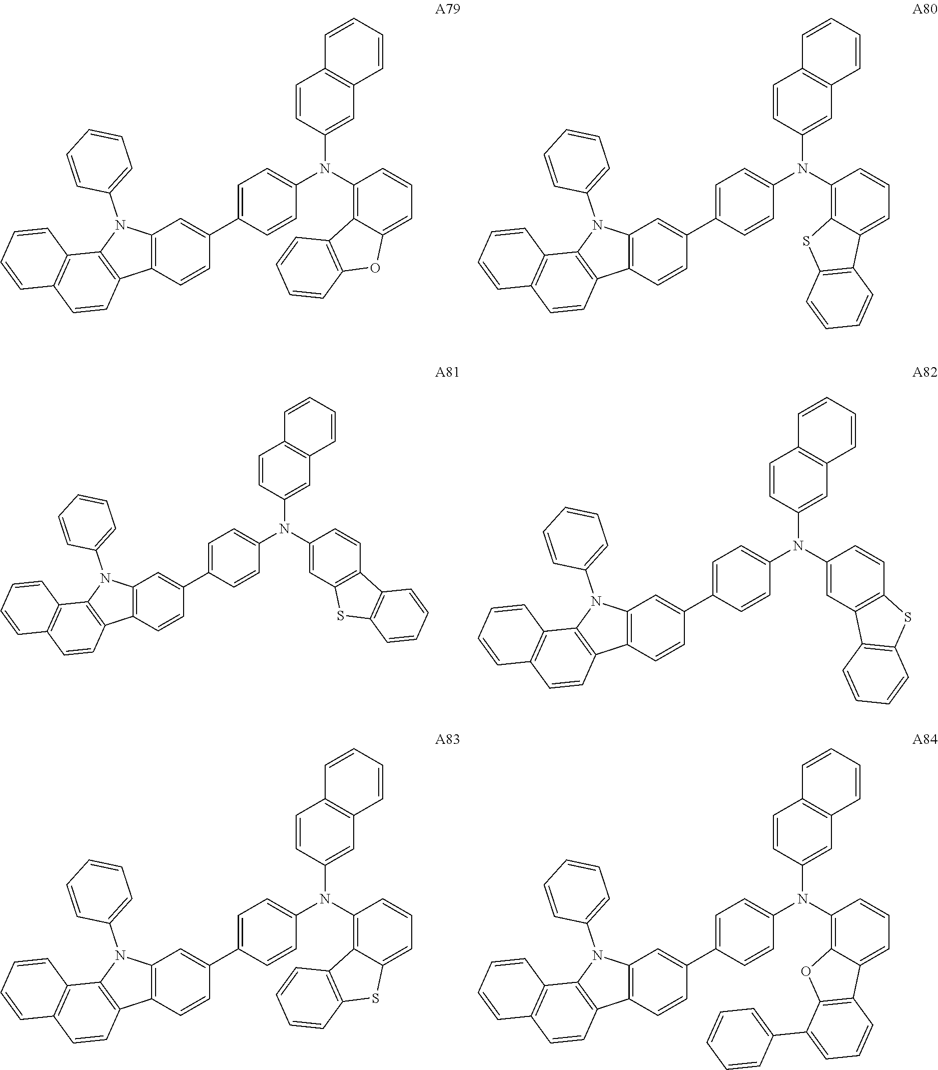 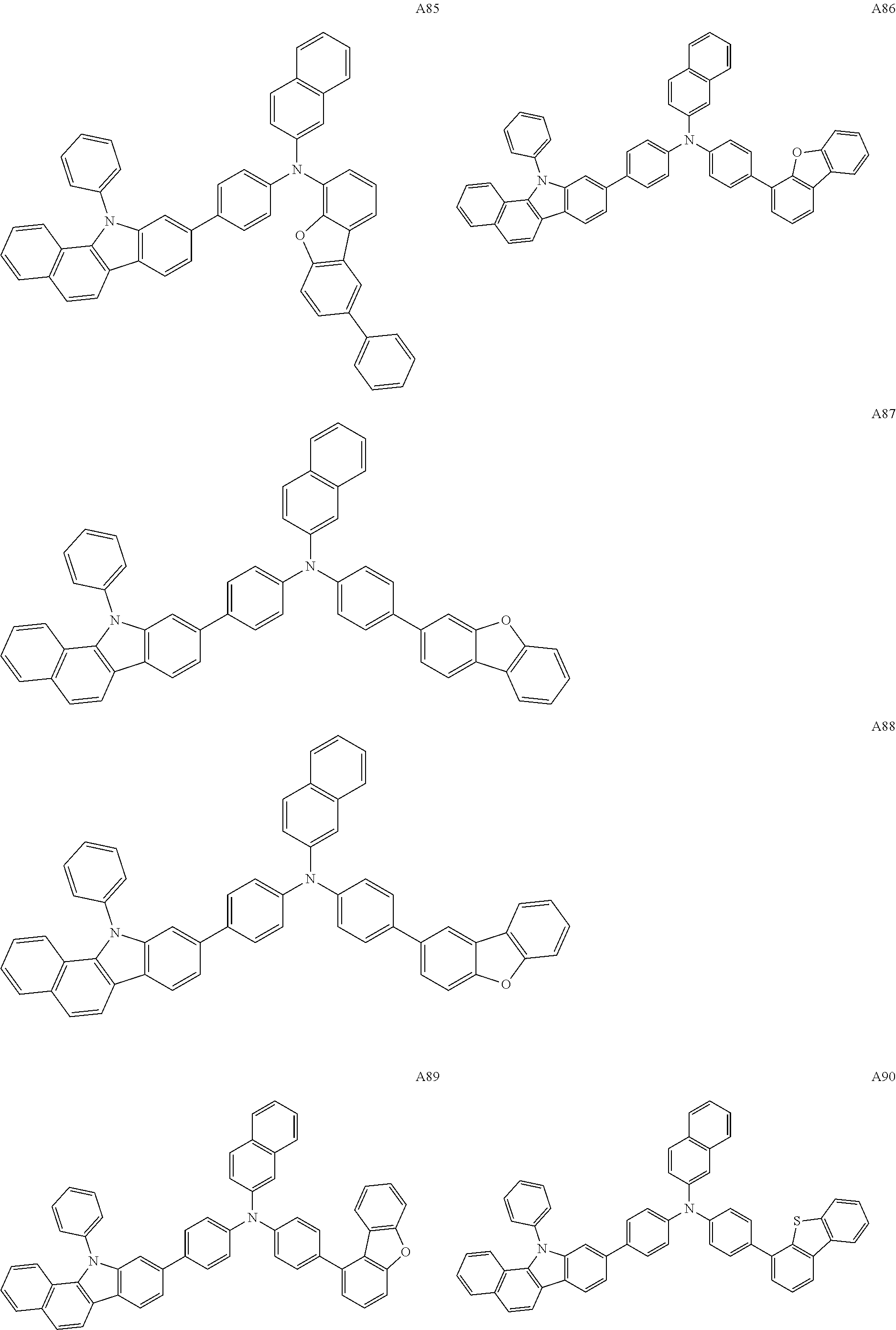 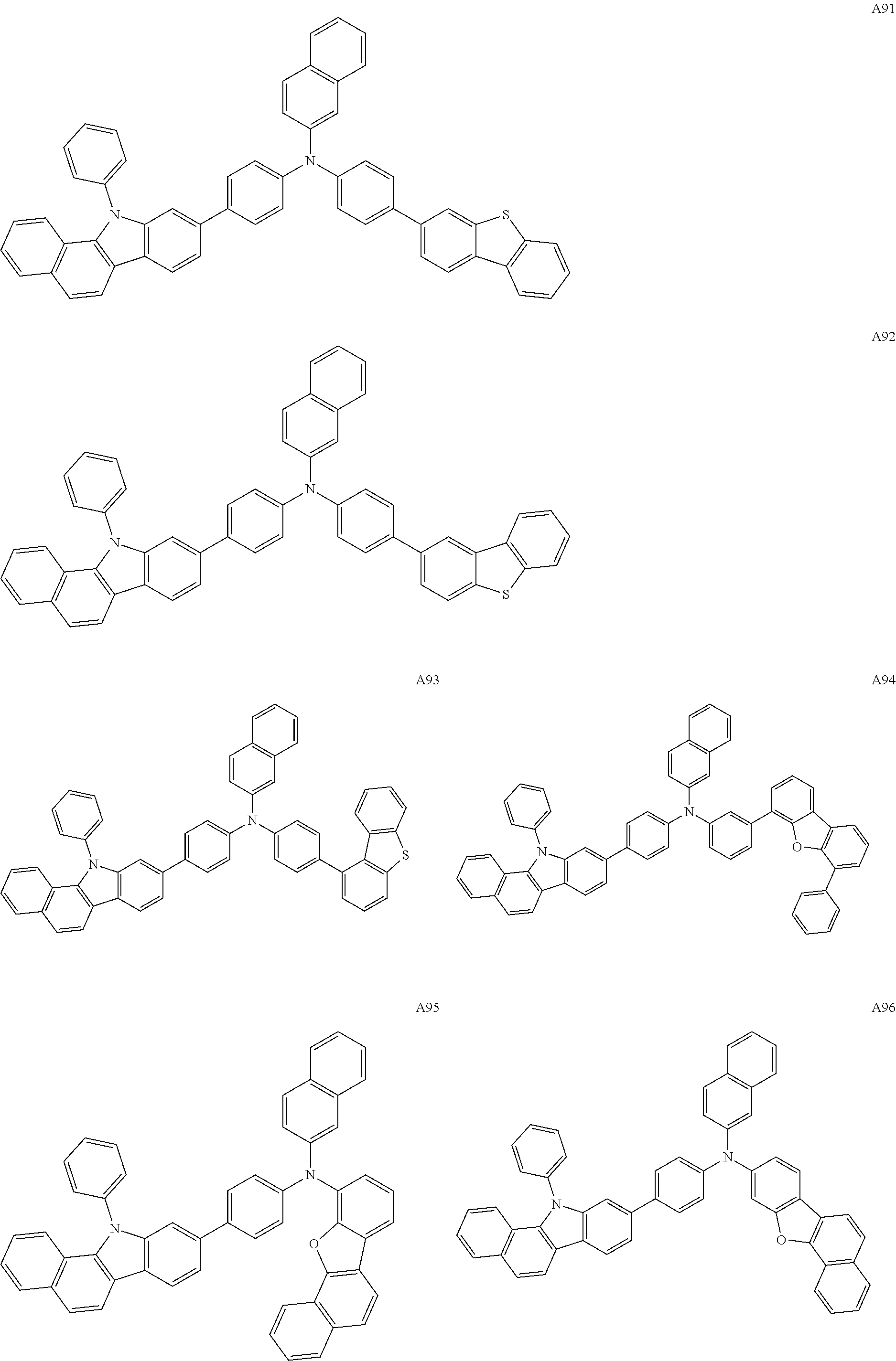 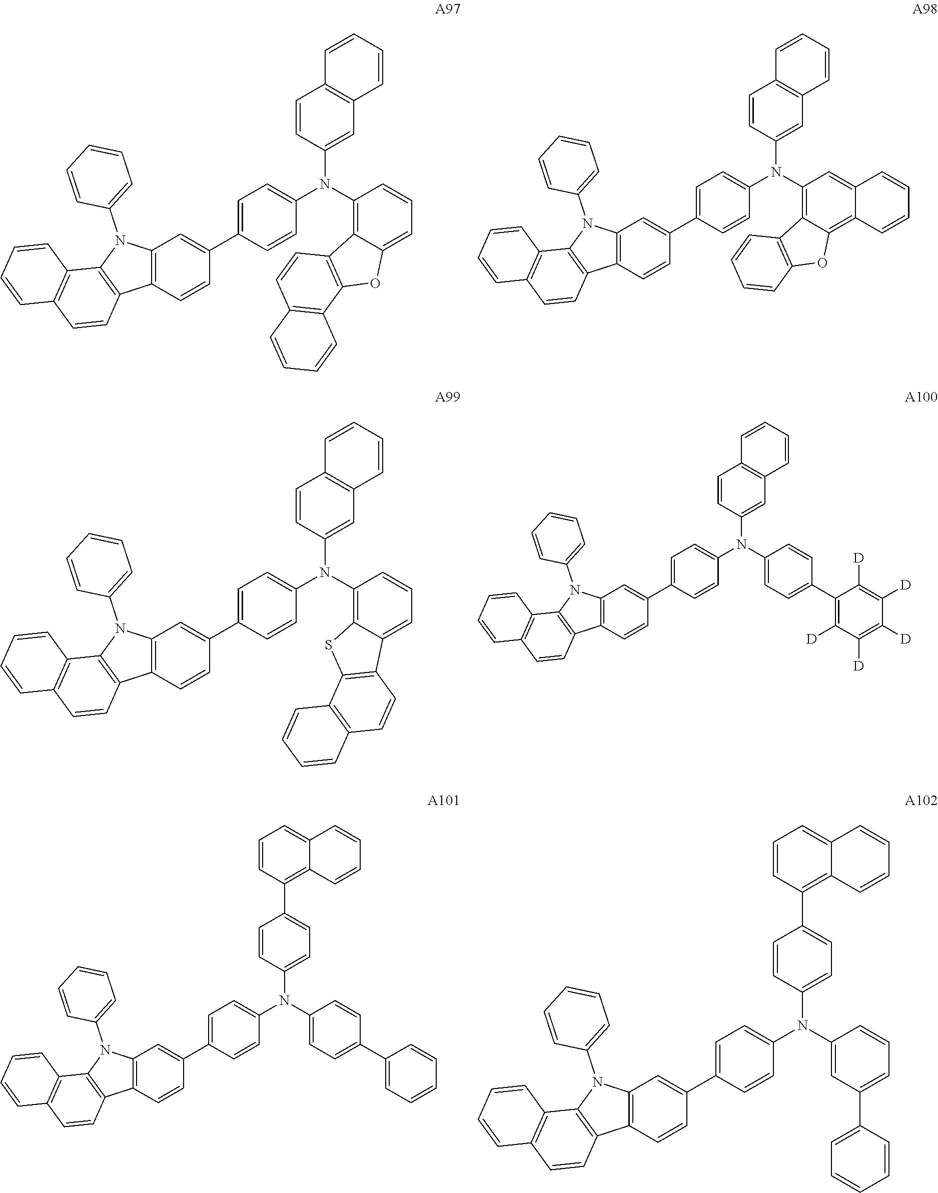 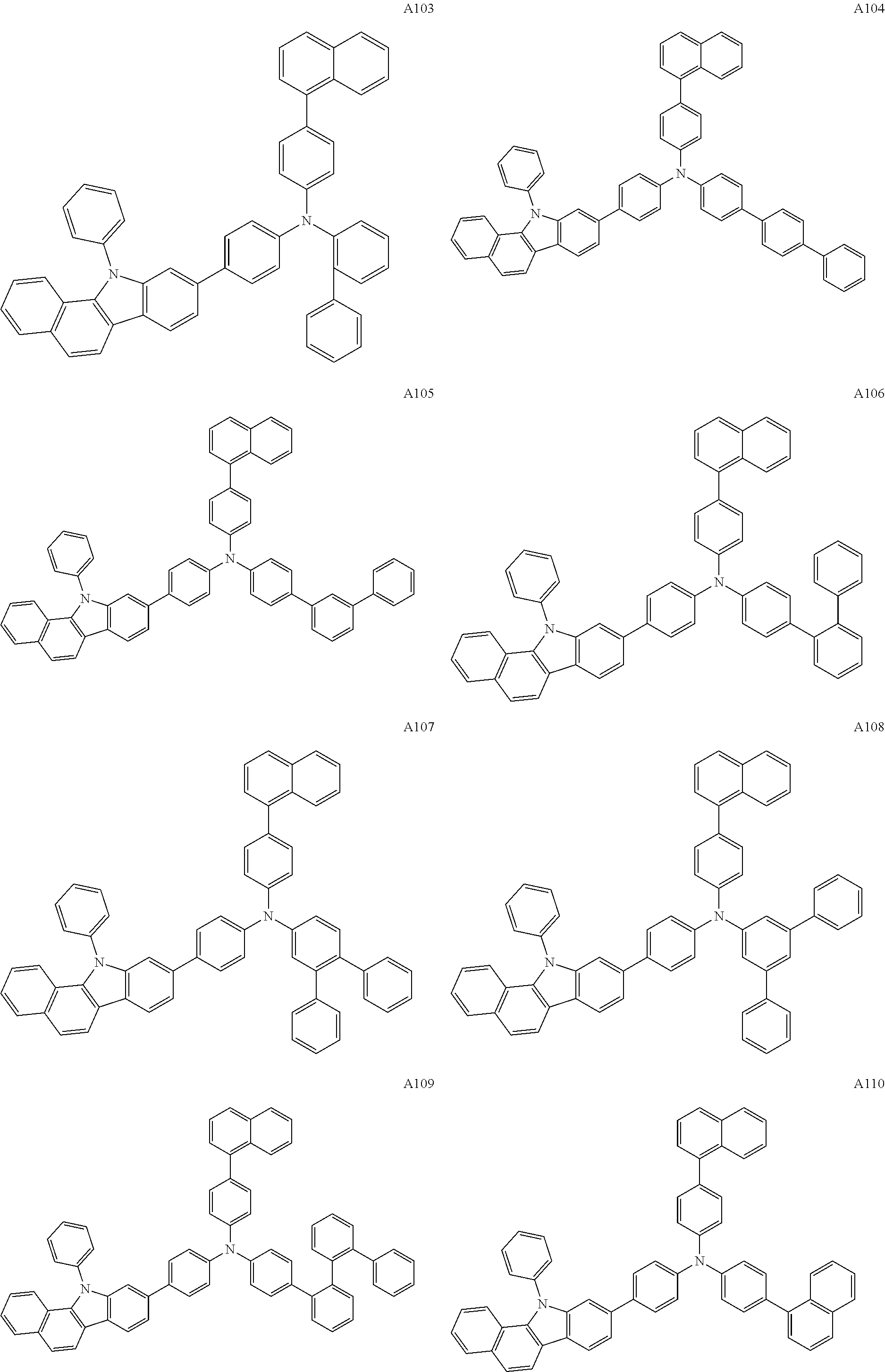 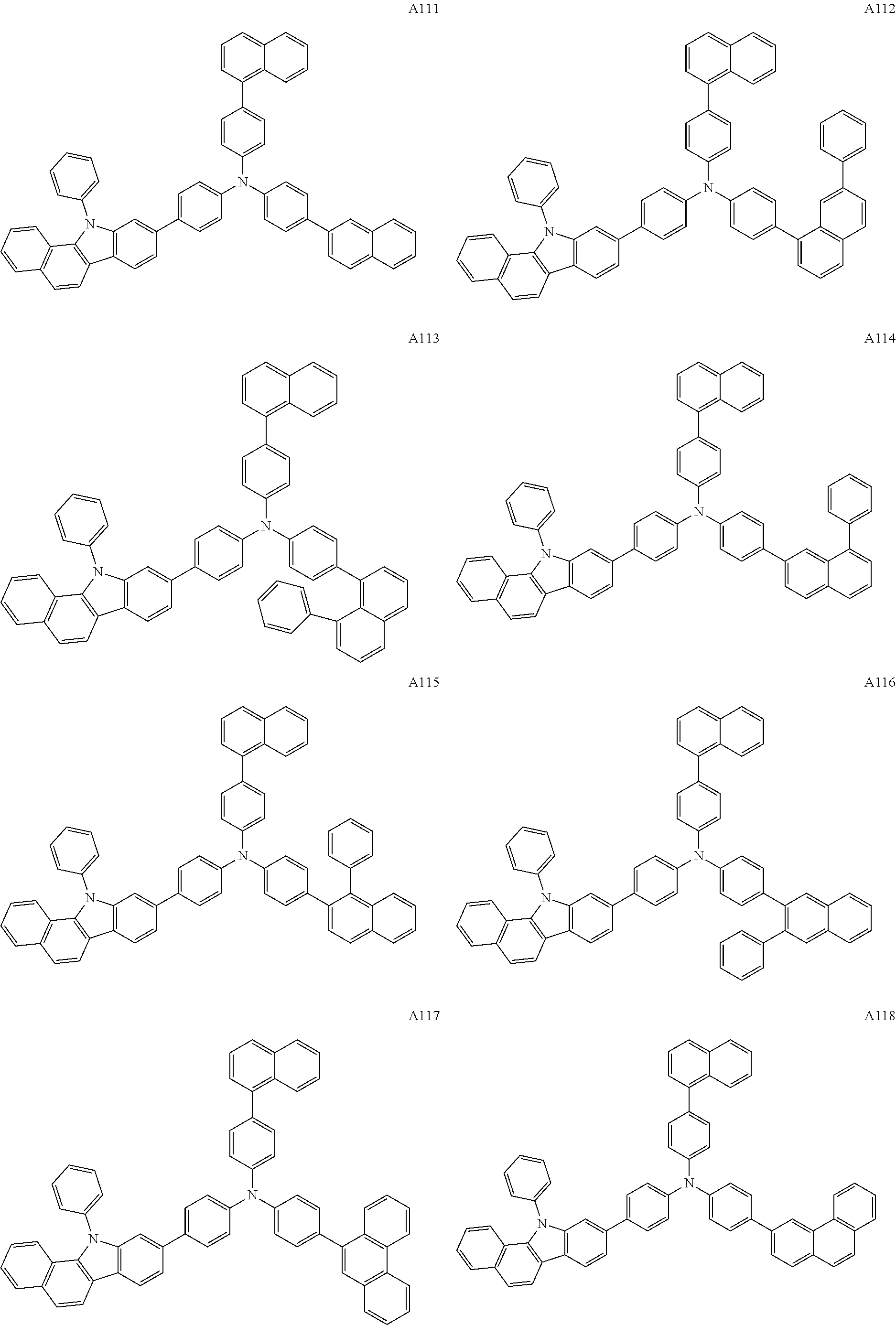 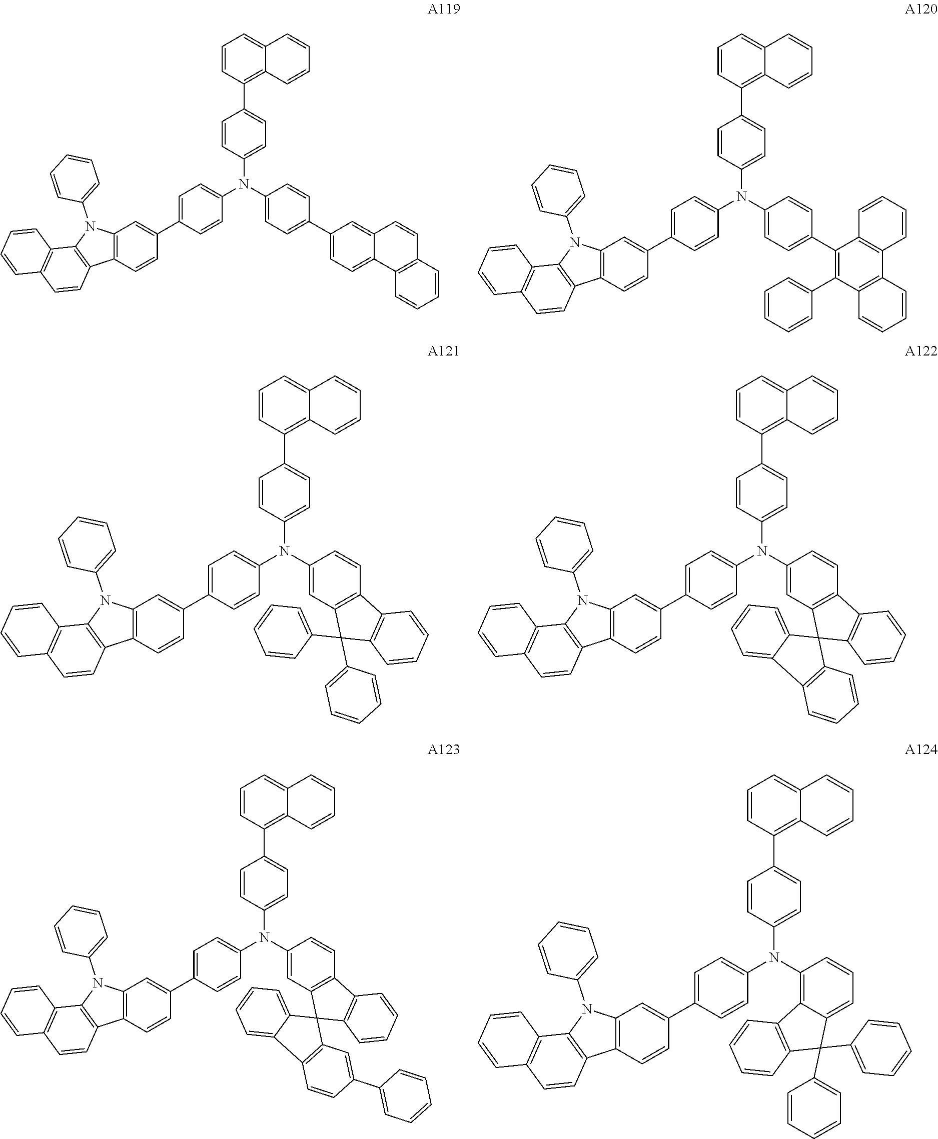 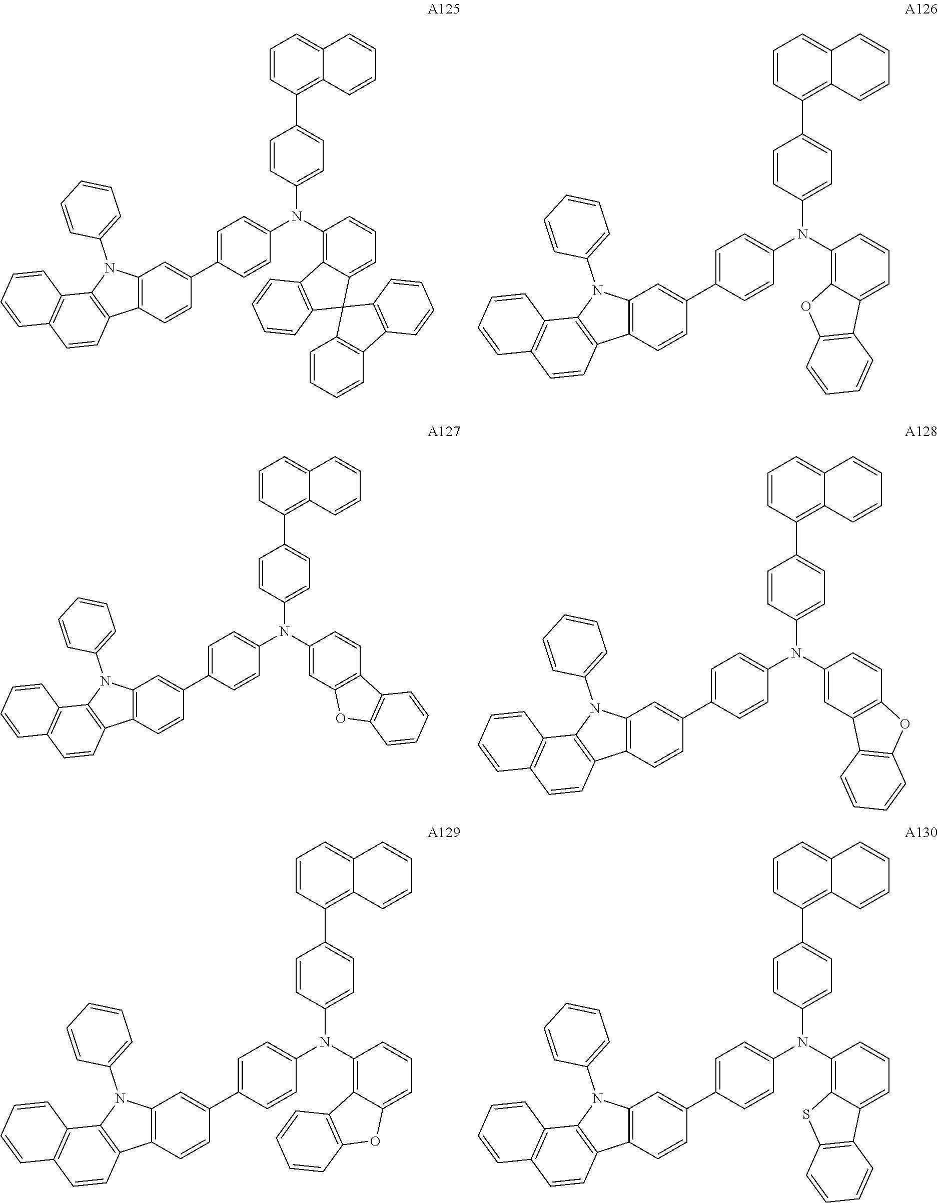 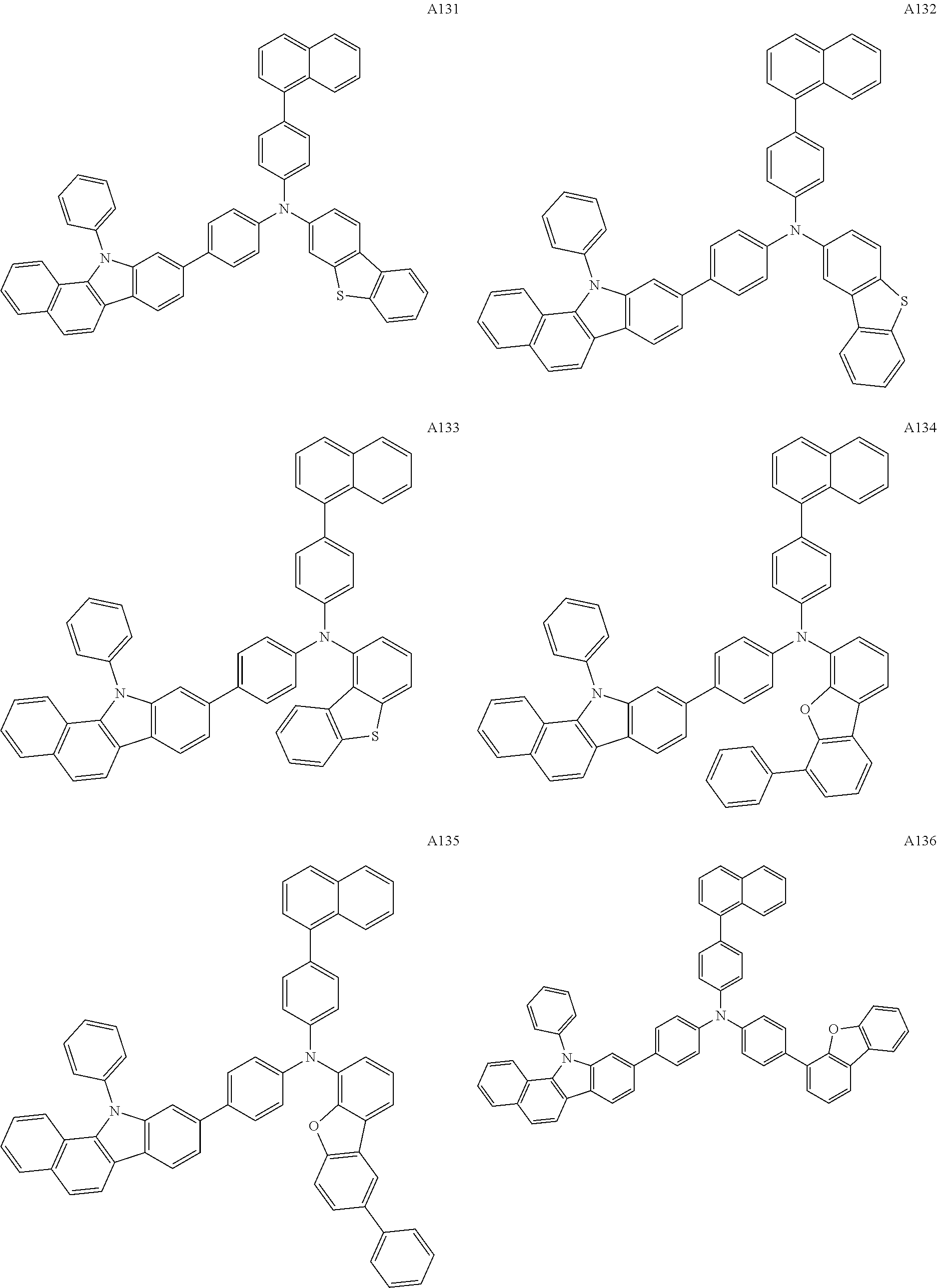 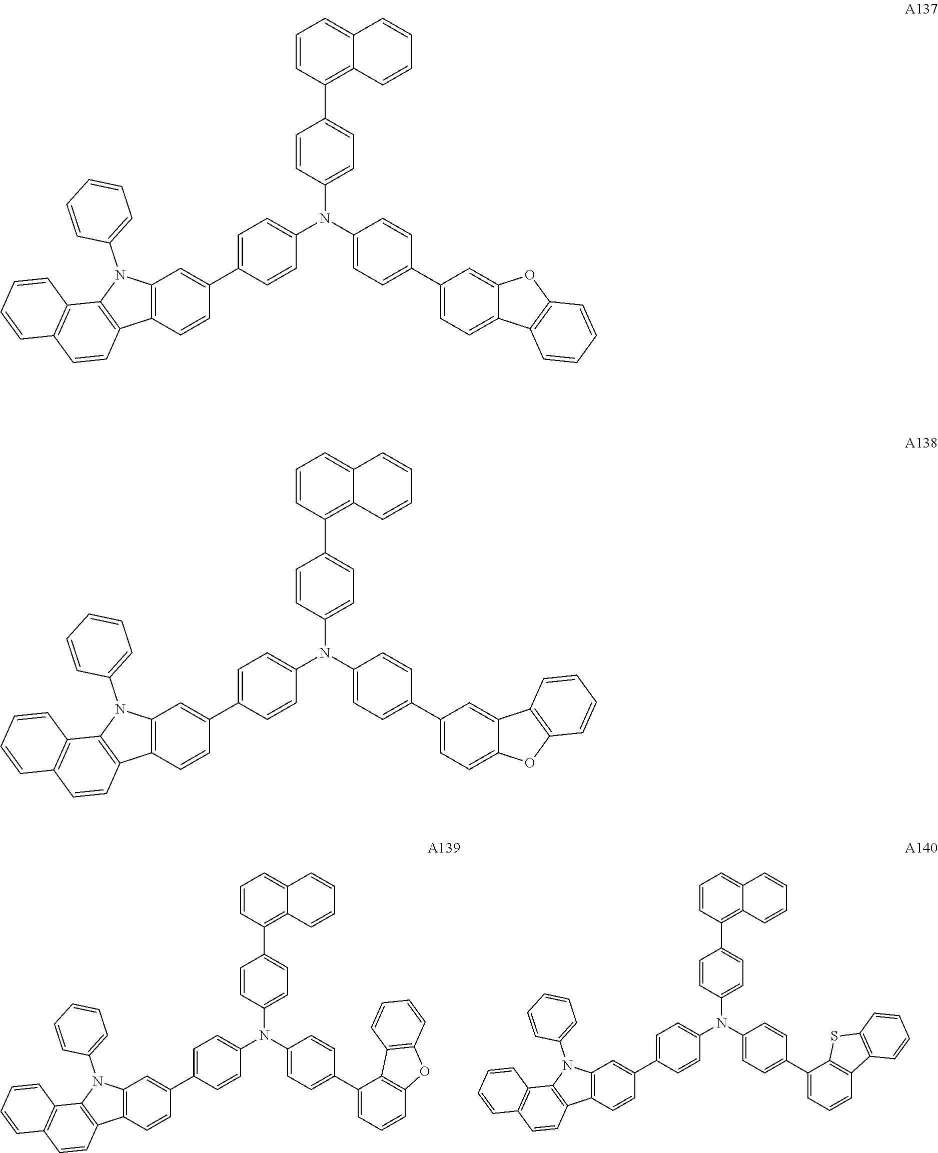 [SMILES: c1ccc(-n2c3cc(-c4ccc(N(c5ccc(-c6ccc7c(c6)oc6ccccc67)cc5)c5ccc(-c6cccc7ccccc67)cc5)cc4)ccc3c3ccc4ccccc4c32)cc1 ; c1ccc(-n2c3cc(-c4ccc(N(c5ccc(-c6ccc7oc8ccccc8c7c6)cc5)c5ccc(-c6cccc7ccccc67)cc5)cc4)ccc3c3ccc4ccccc4c32)cc1 ; c1ccc(-n2c3cc(-c4ccc(N(c5ccc(-c6cccc7ccccc67)cc5)c5ccc(-c6cccc7oc8ccccc8c67)cc5)cc4)ccc3c3ccc4ccccc4c32)cc1 ; c1ccc(-n2c3cc(-c4ccc(N(c5ccc(-c6cccc7ccccc67)cc5)c5ccc(-c6cccc7c6sc6ccccc67)cc5)cc4)ccc3c3ccc4ccccc4c32)cc1] 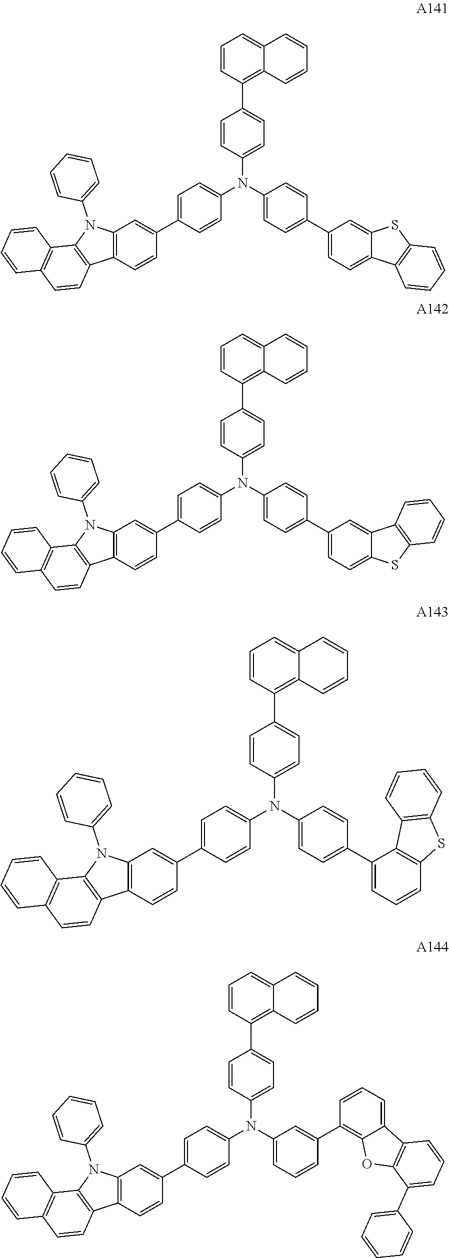 [SMILES: c1ccc(-n2c3cc(-c4ccc(N(c5ccc(-c6ccc7c(c6)sc6ccccc67)cc5)c5ccc(-c6cccc7ccccc67)cc5)cc4)ccc3c3ccc4ccccc4c32)cc1 ; c1ccc(-n2c3cc(-c4ccc(N(c5ccc(-c6ccc7sc8ccccc8c7c6)cc5)c5ccc(-c6cccc7ccccc67)cc5)cc4)ccc3c3ccc4ccccc4c32)cc1 ; c1ccc(-n2c3cc(-c4ccc(N(c5ccc(-c6cccc7ccccc67)cc5)c5ccc(-c6cccc7sc8ccccc8c67)cc5)cc4)ccc3c3ccc4ccccc4c32)cc1 ; c1ccc(-c2cccc3c2oc2c(-c4cccc(N(c5ccc(-c6ccc7c8ccc9ccccc9c8n(-c8ccccc8)c7c6)cc5)c5ccc(-c6cccc7ccccc67)cc5)c4)cccc23)cc1] 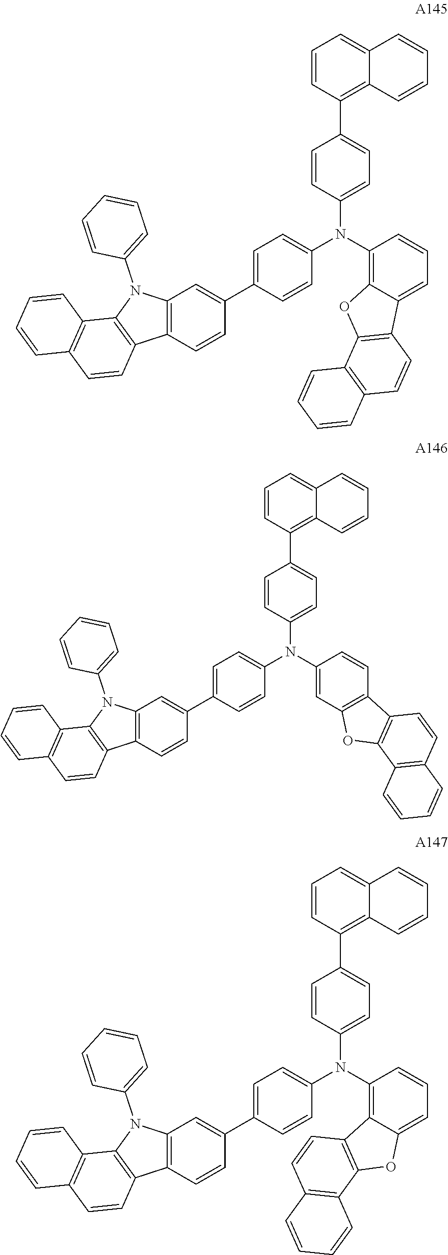 [SMILES: c1ccc(-n2c3cc(-c4ccc(N(c5ccc(-c6cccc7ccccc67)cc5)c5cccc6c5oc5c7ccccc7ccc65)cc4)ccc3c3ccc4ccccc4c32)cc1 ; c1ccc(-n2c3cc(-c4ccc(N(c5ccc(-c6cccc7ccccc67)cc5)c5ccc6c(c5)oc5c7ccccc7ccc65)cc4)ccc3c3ccc4ccccc4c32)cc1 ; c1ccc(-n2c3cc(-c4ccc(N(c5ccc(-c6cccc7ccccc67)cc5)c5cccc6oc7c8ccccc8ccc7c56)cc4)ccc3c3ccc4ccccc4c32)cc1] 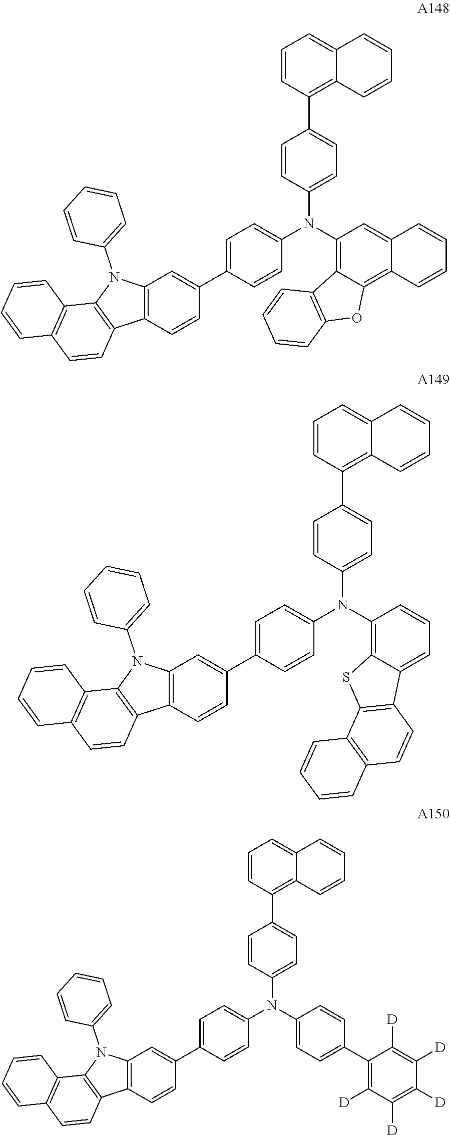 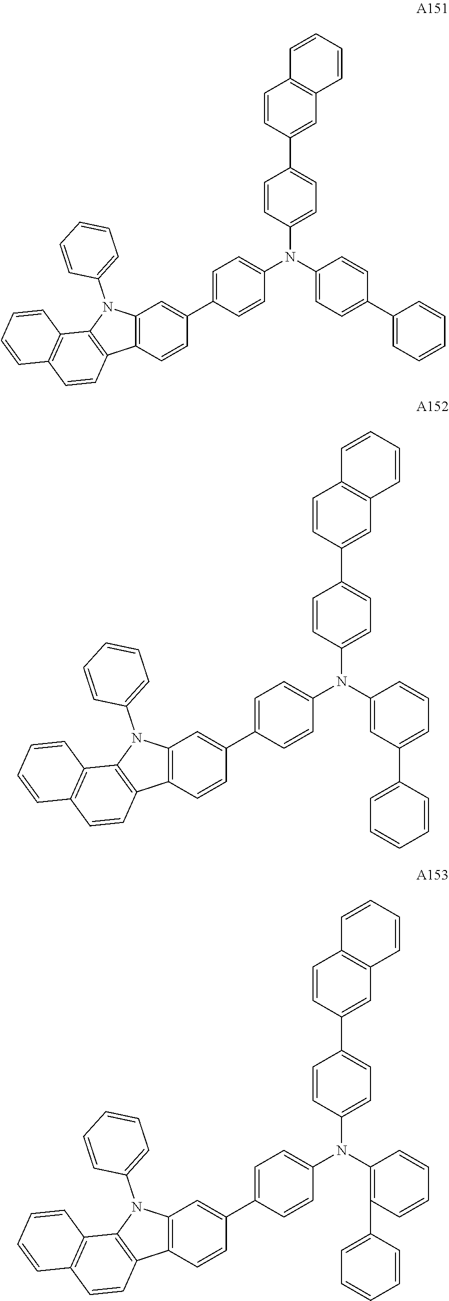 [SMILES: c1ccc(-c2ccc(N(c3ccc(-c4ccc5ccccc5c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5ccccc5c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4ccccc4c3)cc2)c2ccc(-c3ccc4c5ccc6ccccc6c5n(-c5ccccc5)c4c3)cc2)cc1] 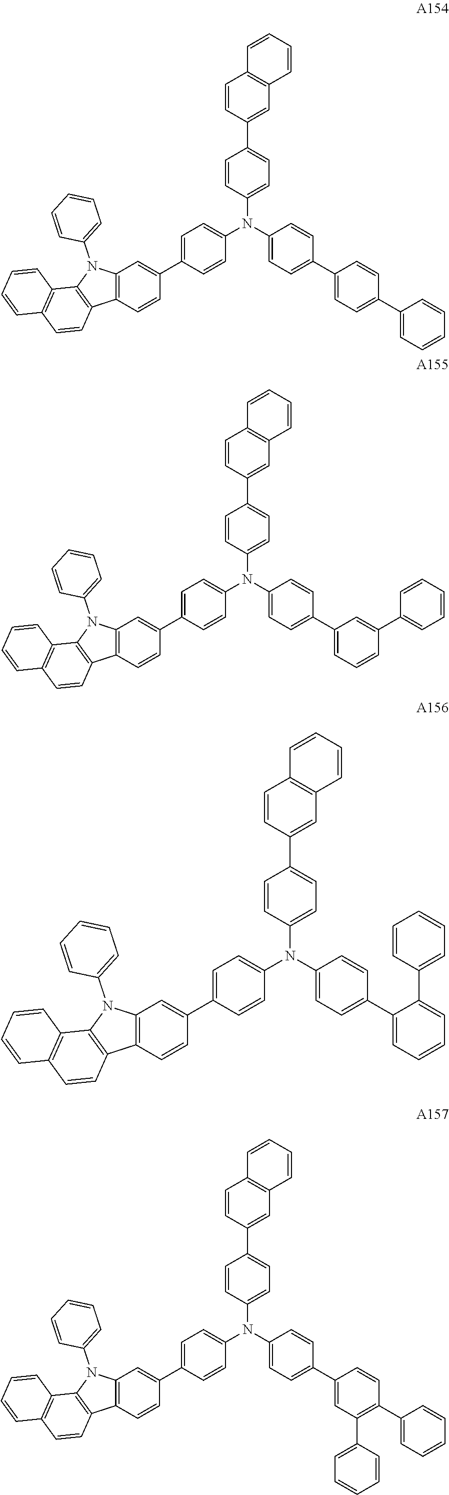 [SMILES: c1ccc(-c2ccc(-c3ccc(N(c4ccc(-c5ccc6ccccc6c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6ccccc6c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5ccccc5c4)cc3)c3ccc(-c4ccc5c6ccc7ccccc7c6n(-c6ccccc6)c5c4)cc3)cc2)cc1 ; c1ccc(-c2ccc(-c3ccc(N(c4ccc(-c5ccc6ccccc6c5)cc4)c4ccc(-c5ccc6c7ccc8ccccc8c7n(-c7ccccc7)c6c5)cc4)cc3)cc2-c2ccccc2)cc1] 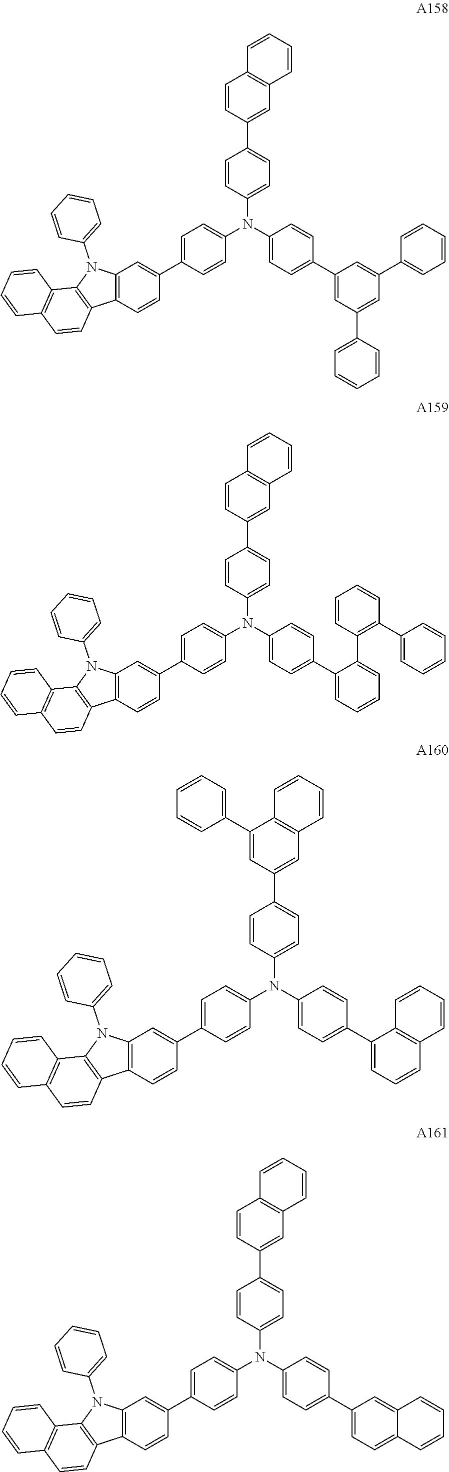 [SMILES: c1ccc(-c2cc(-c3ccccc3)cc(-c3ccc(N(c4ccc(-c5ccc6ccccc6c5)cc4)c4ccc(-c5ccc6c7ccc8ccccc8c7n(-c7ccccc7)c6c5)cc4)cc3)c2)cc1 ; c1ccc(-c2ccccc2-c2ccccc2-c2ccc(N(c3ccc(-c4ccc5ccccc5c4)cc3)c3ccc(-c4ccc5c6ccc7ccccc7c6n(-c6ccccc6)c5c4)cc3)cc2)cc1 ; c1ccc(-c2cc(-c3ccc(N(c4ccc(-c5ccc6c7ccc8ccccc8c7n(-c7ccccc7)c6c5)cc4)c4ccc(-c5cccc6ccccc56)cc4)cc3)cc3ccccc23)cc1 ; c1ccc(-n2c3cc(-c4ccc(N(c5ccc(-c6ccc7ccccc7c6)cc5)c5ccc(-c6ccc7ccccc7c6)cc5)cc4)ccc3c3ccc4ccccc4c32)cc1] 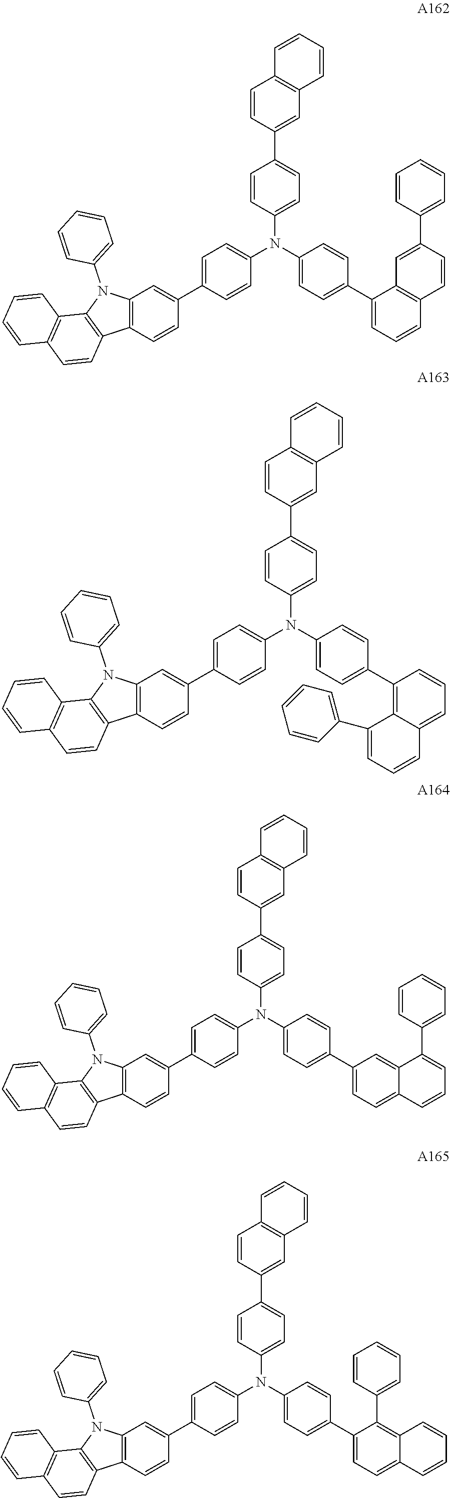 [SMILES: c1ccc(-c2ccc3cccc(-c4ccc(N(c5ccc(-c6ccc7ccccc7c6)cc5)c5ccc(-c6ccc7c8ccc9ccccc9c8n(-c8ccccc8)c7c6)cc5)cc4)c3c2)cc1 ; c1ccc(-c2cccc3cccc(-c4ccc(N(c5ccc(-c6ccc7ccccc7c6)cc5)c5ccc(-c6ccc7c8ccc9ccccc9c8n(-c8ccccc8)c7c6)cc5)cc4)c23)cc1 ; c1ccc(-c2cccc3ccc(-c4ccc(N(c5ccc(-c6ccc7ccccc7c6)cc5)c5ccc(-c6ccc7c8ccc9ccccc9c8n(-c8ccccc8)c7c6)cc5)cc4)cc23)cc1 ; c1ccc(-c2c(-c3ccc(N(c4ccc(-c5ccc6ccccc6c5)cc4)c4ccc(-c5ccc6c7ccc8ccccc8c7n(-c7ccccc7)c6c5)cc4)cc3)ccc3ccccc23)cc1] 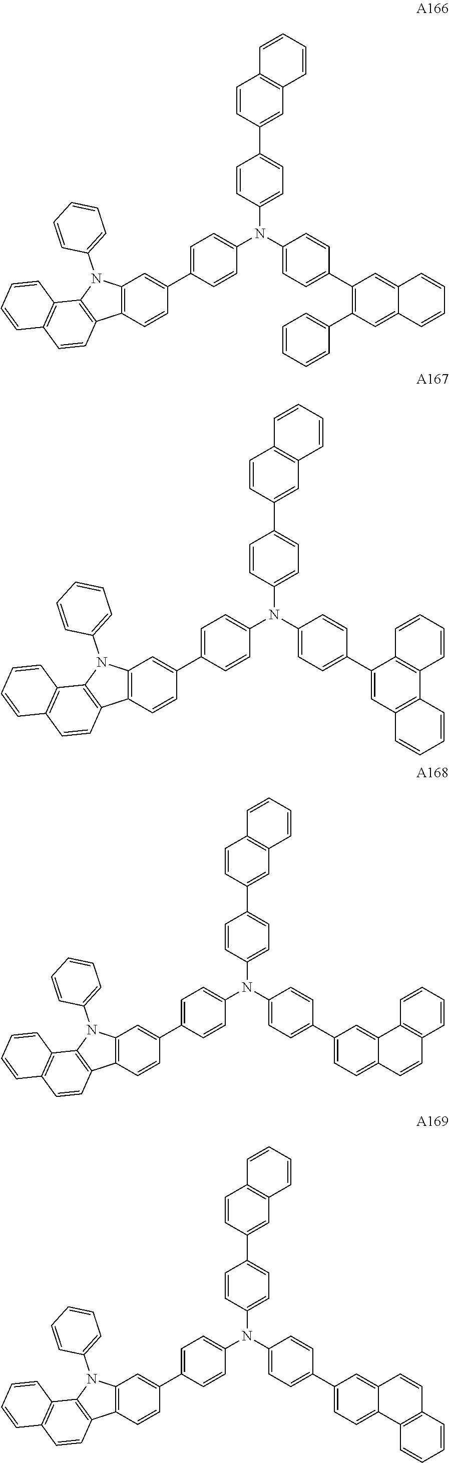 [SMILES: c1ccc(-c2cc3ccccc3cc2-c2ccc(N(c3ccc(-c4ccc5ccccc5c4)cc3)c3ccc(-c4ccc5c6ccc7ccccc7c6n(-c6ccccc6)c5c4)cc3)cc2)cc1 ; c1ccc(-n2c3cc(-c4ccc(N(c5ccc(-c6ccc7ccccc7c6)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6ccc7ccc8ccccc8c7c6)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6ccc7c(ccc8ccccc87)c6)cc5)cc4)ccc3c3ccc4ccccc4c32)cc1] 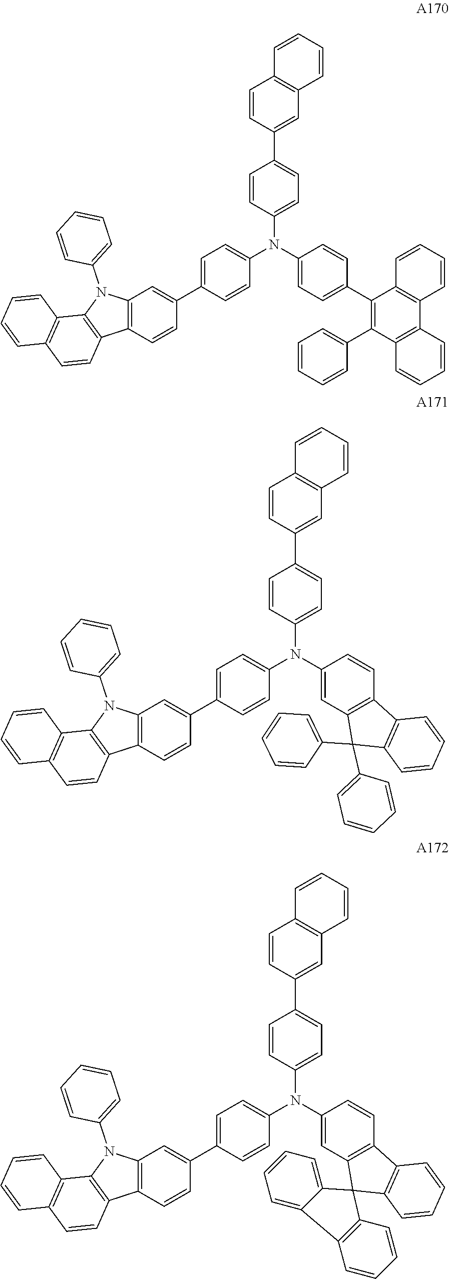 [SMILES: c1ccc(-c2c(-c3ccc(N(c4ccc(-c5ccc6ccccc6c5)cc4)c4ccc(-c5ccc6c7ccc8ccccc8c7n(-c7ccccc7)c6c5)cc4)cc3)c3ccccc3c3ccccc23)cc1 ; c1ccc(-n2c3cc(-c4ccc(N(c5ccc(-c6ccc7ccccc7c6)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6ccc7ccccc7c6)cc5)c5ccc6c(c5)C5(c7ccccc7-c7ccccc75)c5ccccc5-6)cc4)ccc3c3ccc4ccccc4c32)cc1] 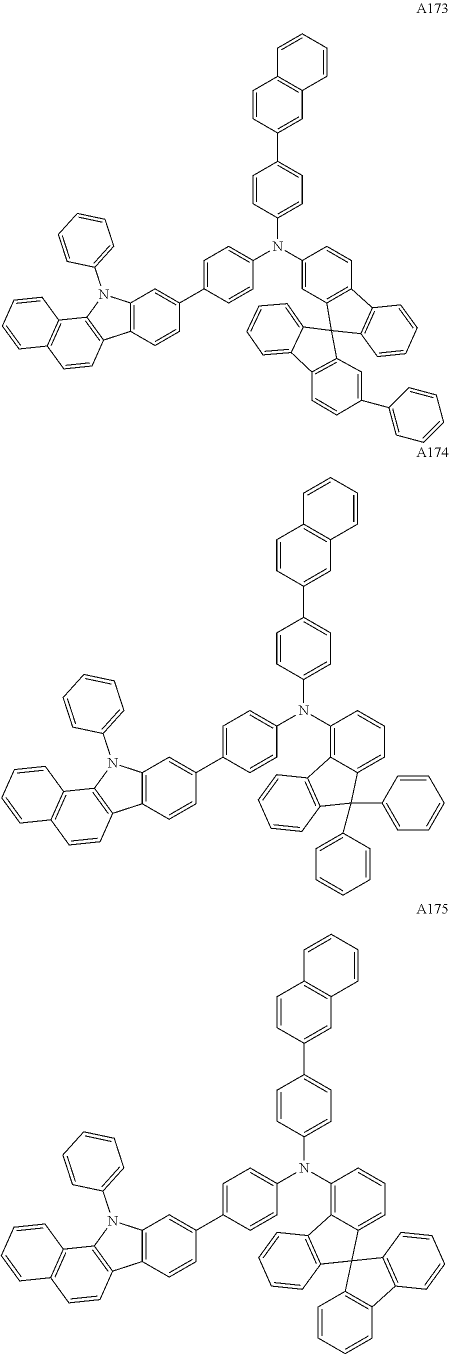 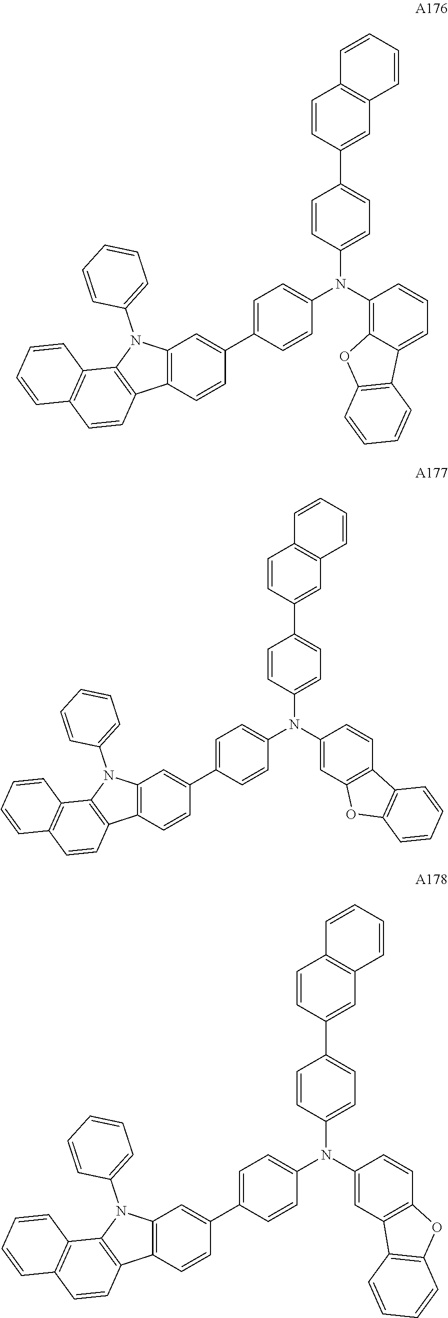 [SMILES: c1ccc(-n2c3cc(-c4ccc(N(c5ccc(-c6ccc7ccccc7c6)cc5)c5cccc6c5oc5ccccc56)cc4)ccc3c3ccc4ccccc4c32)cc1 ; c1ccc(-n2c3cc(-c4ccc(N(c5ccc(-c6ccc7ccccc7c6)cc5)c5ccc6c(c5)oc5ccccc56)cc4)ccc3c3ccc4ccccc4c32)cc1 ; c1ccc(-n2c3cc(-c4ccc(N(c5ccc(-c6ccc7ccccc7c6)cc5)c5ccc6oc7ccccc7c6c5)cc4)ccc3c3ccc4ccccc4c32)cc1] 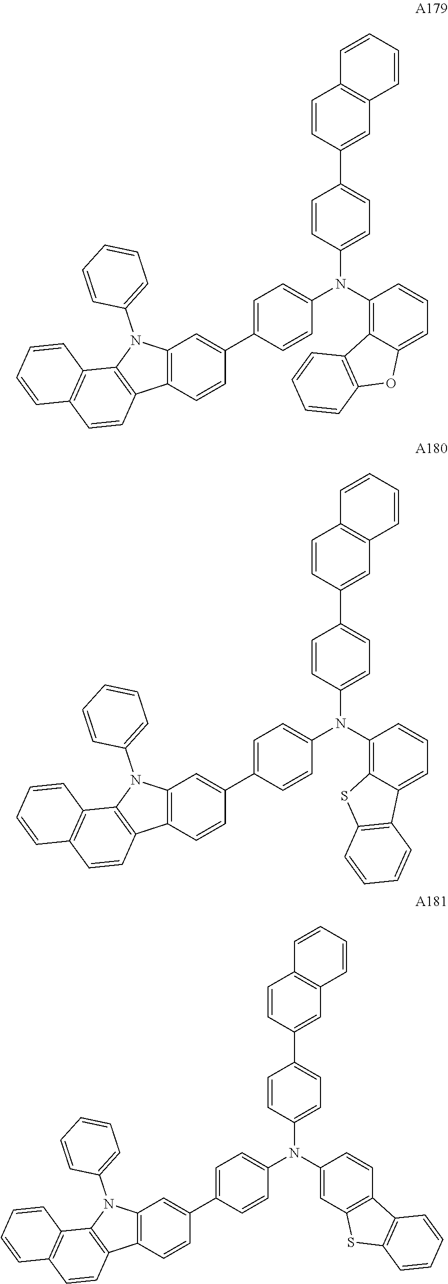 [SMILES: c1ccc(-n2c3cc(-c4ccc(N(c5ccc(-c6ccc7ccccc7c6)cc5)c5cccc6oc7ccccc7c56)cc4)ccc3c3ccc4ccccc4c32)cc1 ; c1ccc(-n2c3cc(-c4ccc(N(c5ccc(-c6ccc7ccccc7c6)cc5)c5cccc6c5sc5ccccc56)cc4)ccc3c3ccc4ccccc4c32)cc1 ; c1ccc(-n2c3cc(-c4ccc(N(c5ccc(-c6ccc7ccccc7c6)cc5)c5ccc6c(c5)sc5ccccc56)cc4)ccc3c3ccc4ccccc4c32)cc1] 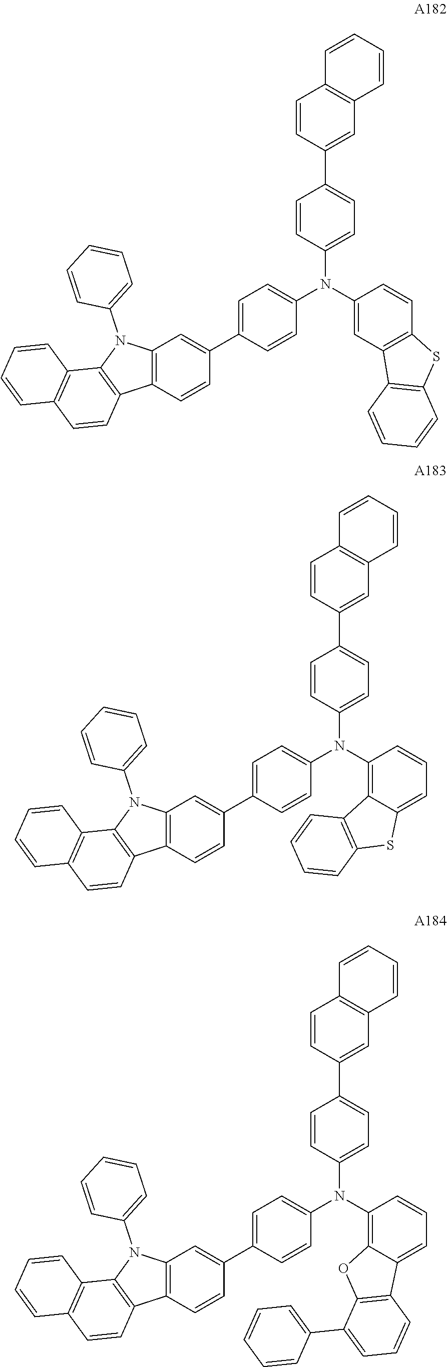 [SMILES: c1ccc(-n2c3cc(-c4ccc(N(c5ccc(-c6ccc7ccccc7c6)cc5)c5ccc6sc7ccccc7c6c5)cc4)ccc3c3ccc4ccccc4c32)cc1 ; c1ccc(-n2c3cc(-c4ccc(N(c5ccc(-c6ccc7ccccc7c6)cc5)c5cccc6sc7ccccc7c56)cc4)ccc3c3ccc4ccccc4c32)cc1 ; c1ccc(-c2cccc3c2oc2c(N(c4ccc(-c5ccc6ccccc6c5)cc4)c4ccc(-c5ccc6c7ccc8ccccc8c7n(-c7ccccc7)c6c5)cc4)cccc23)cc1] 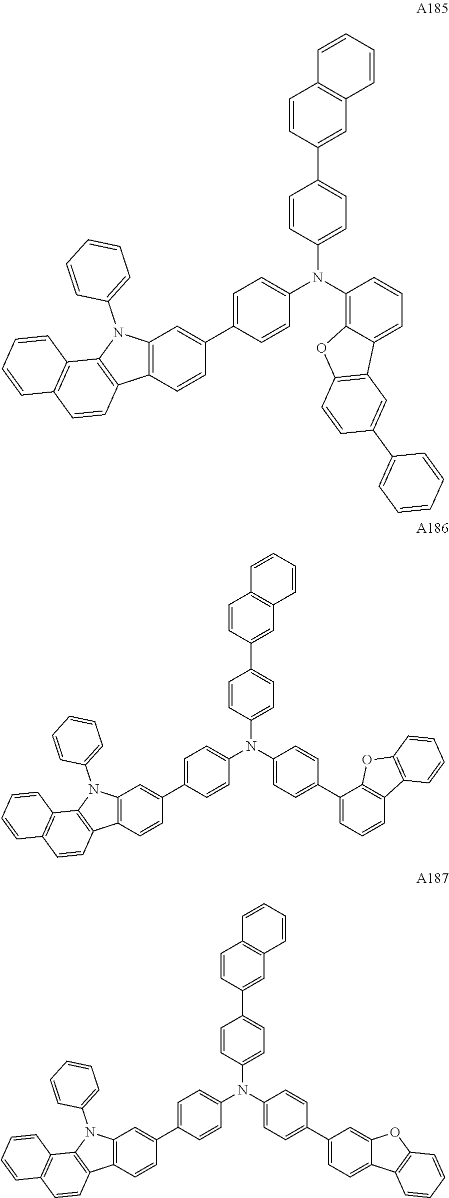 [SMILES: c1ccc(-c2ccc3oc4c(N(c5ccc(-c6ccc7ccccc7c6)cc5)c5ccc(-c6ccc7c8ccc9ccccc9c8n(-c8ccccc8)c7c6)cc5)cccc4c3c2)cc1 ; c1ccc(-n2c3cc(-c4ccc(N(c5ccc(-c6ccc7ccccc7c6)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6ccc7c(c6)oc6ccccc67)cc5)cc4)ccc3c3ccc4ccccc4c32)cc1] 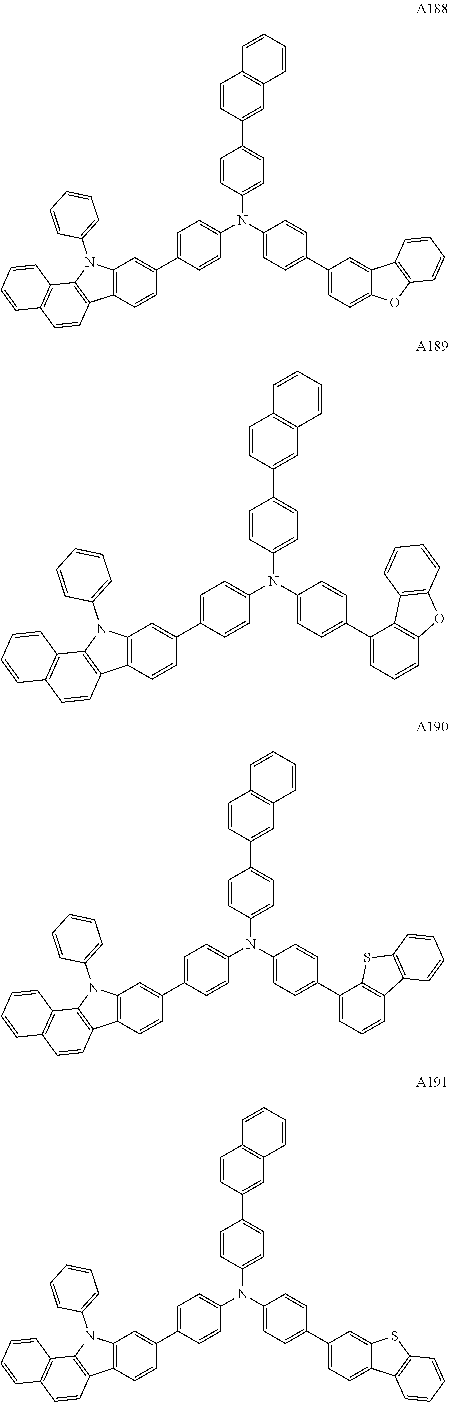 [SMILES: c1ccc(-n2c3cc(-c4ccc(N(c5ccc(-c6ccc7ccccc7c6)cc5)c5ccc(-c6ccc7oc8ccccc8c7c6)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6cccc7oc8ccccc8c67)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6cccc7c6sc6ccccc67)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6ccc7c(c6)sc6ccccc67)cc5)cc4)ccc3c3ccc4ccccc4c32)cc1] 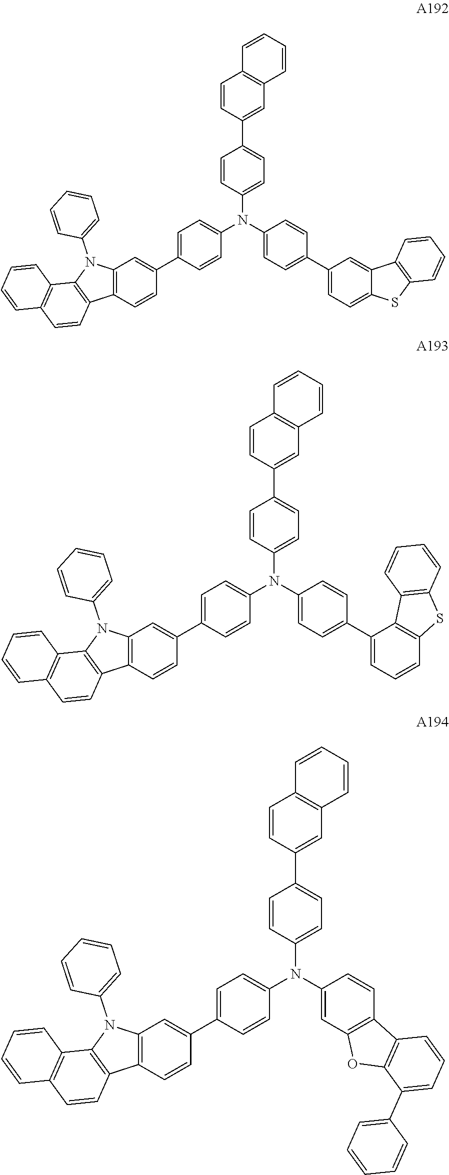 [SMILES: c1ccc(-n2c3cc(-c4ccc(N(c5ccc(-c6ccc7ccccc7c6)cc5)c5ccc(-c6ccc7sc8ccccc8c7c6)cc5)cc4)ccc3c3ccc4ccccc4c32)cc1 ; c1ccc(-n2c3cc(-c4ccc(N(c5ccc(-c6ccc7ccccc7c6)cc5)c5ccc(-c6cccc7sc8ccccc8c67)cc5)cc4)ccc3c3ccc4ccccc4c32)cc1 ; c1ccc(-c2cccc3c2oc2cc(N(c4ccc(-c5ccc6ccccc6c5)cc4)c4ccc(-c5ccc6c7ccc8ccccc8c7n(-c7ccccc7)c6c5)cc4)ccc23)cc1] 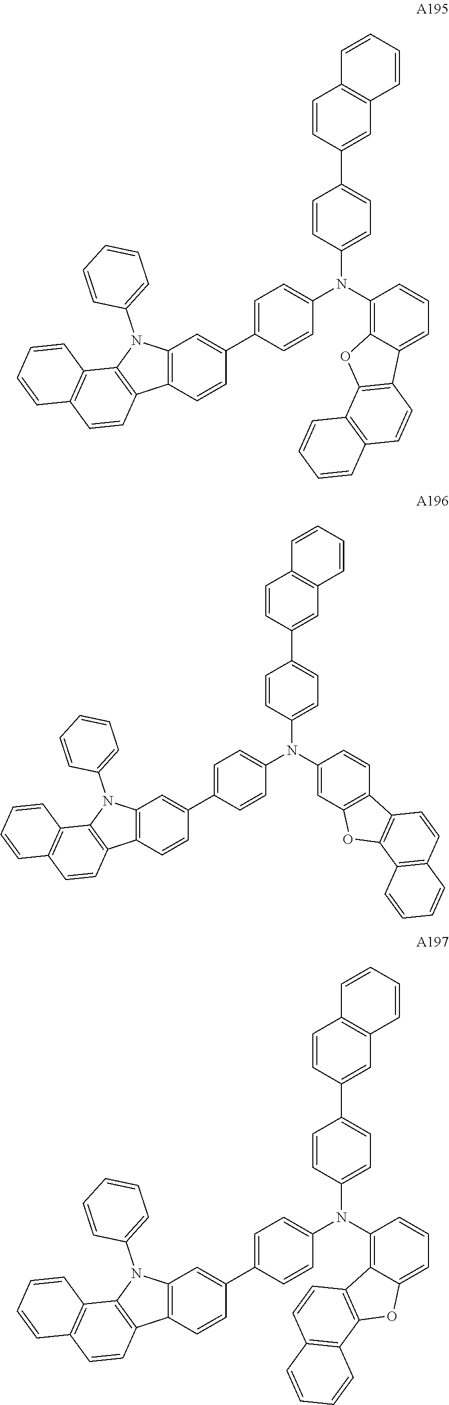 [SMILES: c1ccc(-n2c3cc(-c4ccc(N(c5ccc(-c6ccc7ccccc7c6)cc5)c5cccc6c5oc5c7ccccc7ccc65)cc4)ccc3c3ccc4ccccc4c32)cc1 ; c1ccc(-n2c3cc(-c4ccc(N(c5ccc(-c6ccc7ccccc7c6)cc5)c5ccc6c(c5)oc5c7ccccc7ccc65)cc4)ccc3c3ccc4ccccc4c32)cc1 ; c1ccc(-n2c3cc(-c4ccc(N(c5ccc(-c6ccc7ccccc7c6)cc5)c5cccc6oc7c8ccccc8ccc7c56)cc4)ccc3c3ccc4ccccc4c32)cc1] 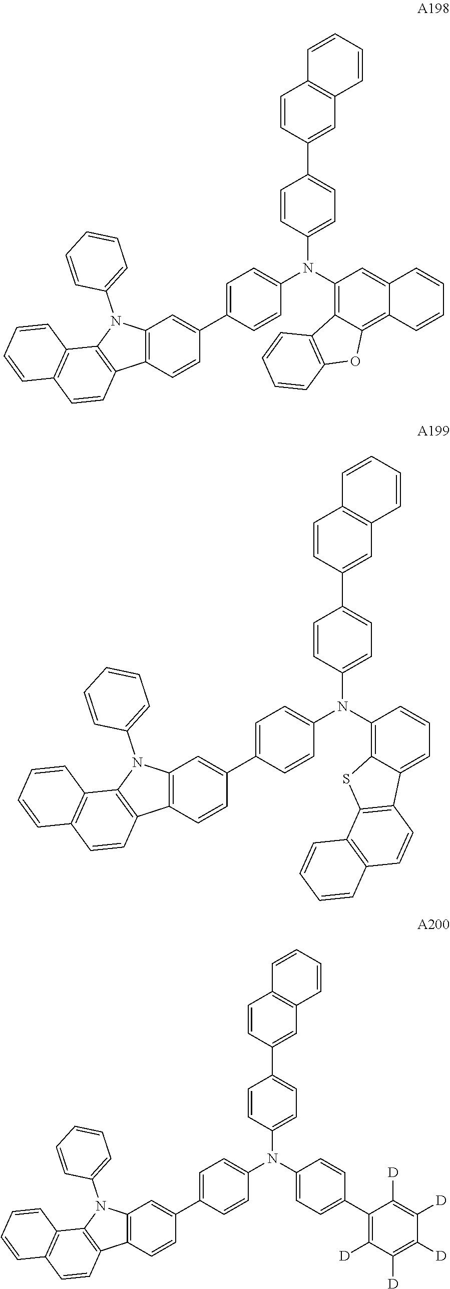 [SMILES: c1ccc(-n2c3cc(-c4ccc(N(c5ccc(-c6ccc7ccccc7c6)cc5)c5cc6ccccc6c6oc7ccccc7c56)cc4)ccc3c3ccc4ccccc4c32)cc1 ; c1ccc(-n2c3cc(-c4ccc(N(c5ccc(-c6ccc7ccccc7c6)cc5)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5ccccc5c4)cc3)c3ccc(-c4ccc5c6ccc7ccccc7c6n(-c6ccccc6)c5c4)cc3)cc2)c([2H])c1[2H]] 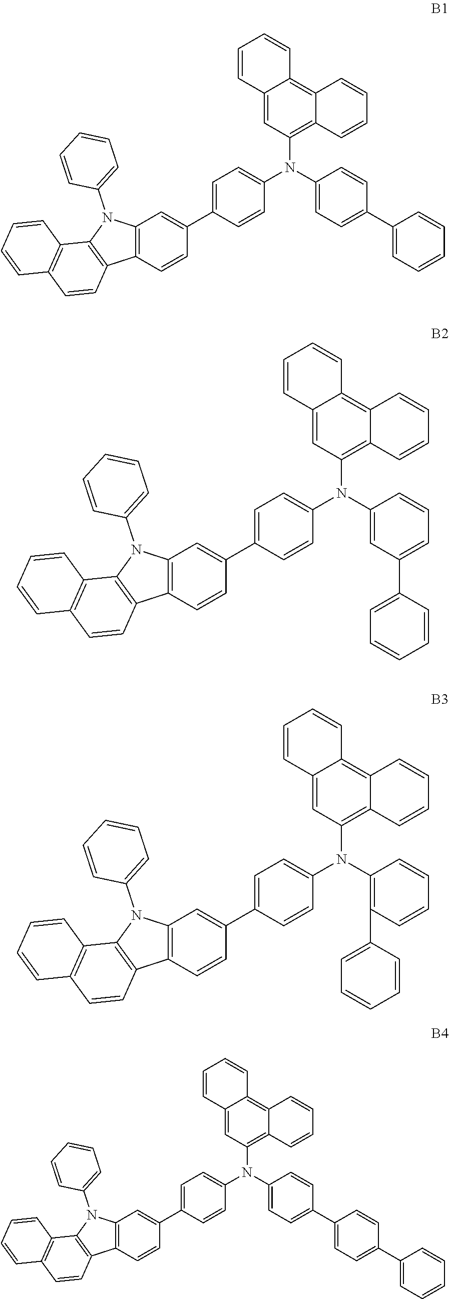 [SMILES: c1ccc(-c2ccc(N(c3ccc(-c4ccc5c6ccc7ccccc7c6n(-c6ccccc6)c5c4)cc3)c3cc4ccccc4c4ccccc34)cc2)cc1 ; c1ccc(-c2cccc(N(c3ccc(-c4ccc5c6ccc7ccccc7c6n(-c6ccccc6)c5c4)cc3)c3cc4ccccc4c4ccccc34)c2)cc1 ; c1ccc(-c2ccccc2N(c2ccc(-c3ccc4c5ccc6ccccc6c5n(-c5ccccc5)c4c3)cc2)c2cc3ccccc3c3ccccc23)cc1 ; c1ccc(-c2ccc(-c3ccc(N(c4ccc(-c5ccc6c7ccc8ccccc8c7n(-c7ccccc7)c6c5)cc4)c4cc5ccccc5c5ccccc45)cc3)cc2)cc1] 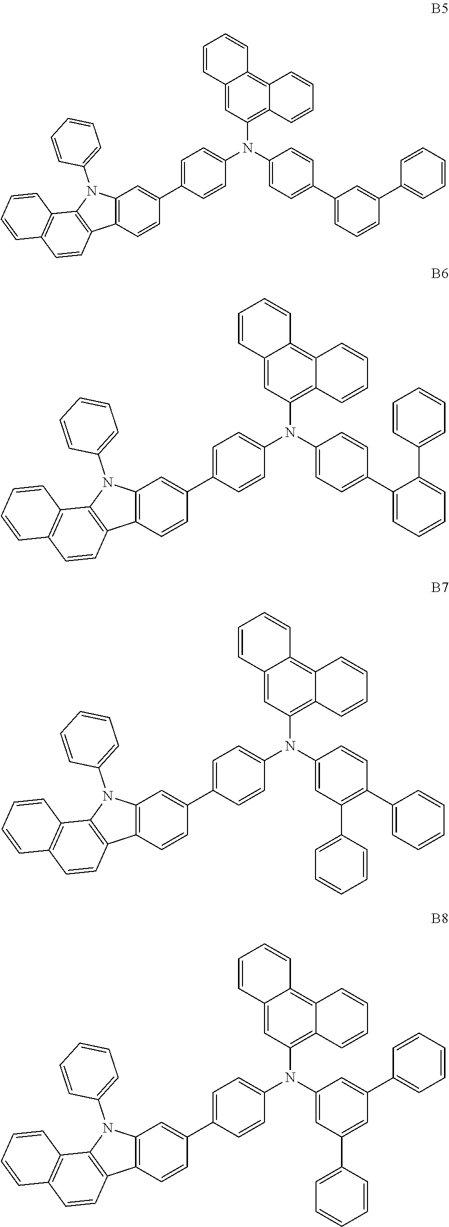 [SMILES: c1ccc(-c2cccc(-c3ccc(N(c4ccc(-c5ccc6c7ccc8ccccc8c7n(-c7ccccc7)c6c5)cc4)c4cc5ccccc5c5ccccc45)cc3)c2)cc1 ; c1ccc(-c2ccccc2-c2ccc(N(c3ccc(-c4ccc5c6ccc7ccccc7c6n(-c6ccccc6)c5c4)cc3)c3cc4ccccc4c4ccccc34)cc2)cc1 ; c1ccc(-c2ccc(N(c3ccc(-c4ccc5c6ccc7ccccc7c6n(-c6ccccc6)c5c4)cc3)c3cc4ccccc4c4ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cc4ccccc4c4ccccc34)c2)cc1] 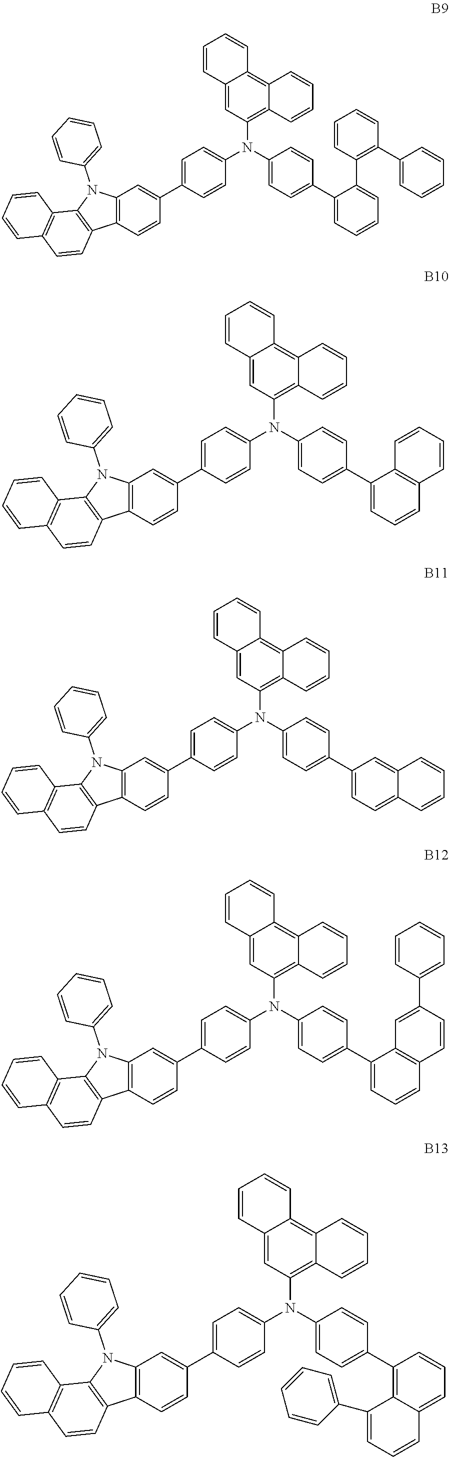 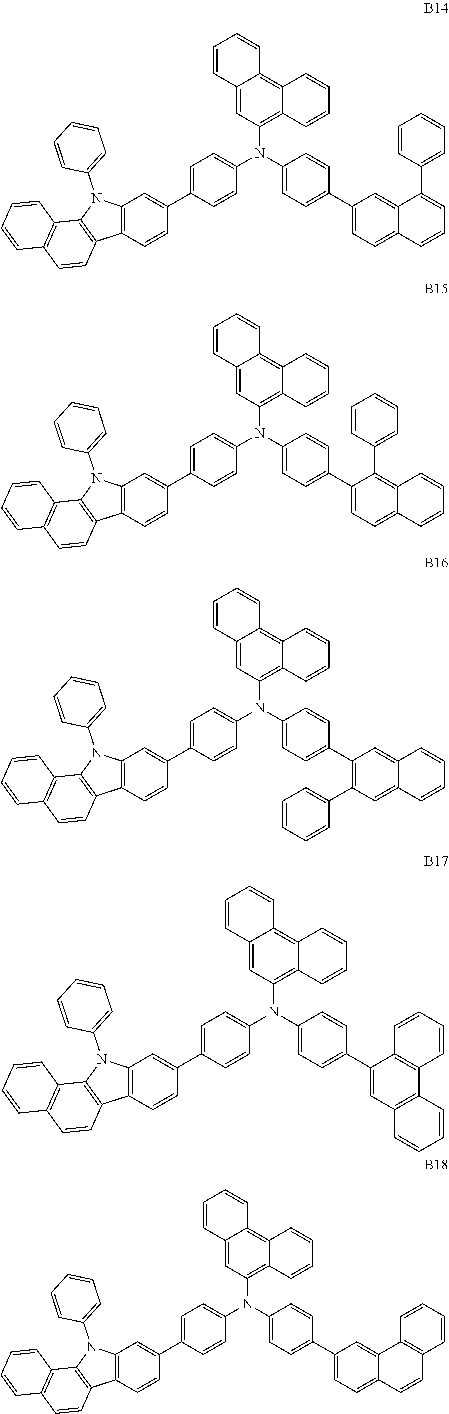 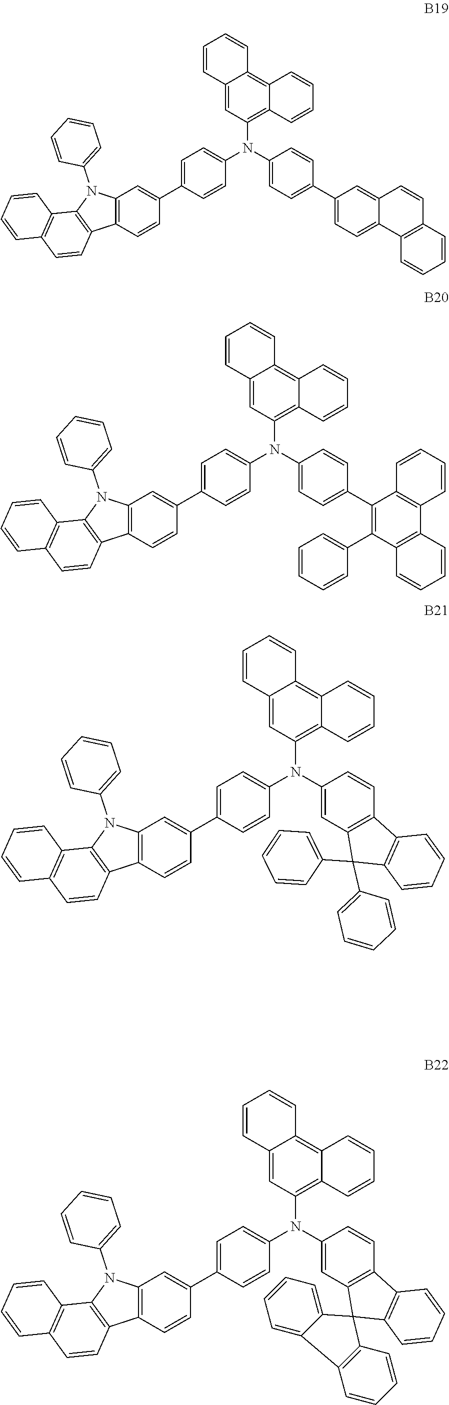 [SMILES: c1ccc(-n2c3cc(-c4ccc(N(c5ccc(-c6ccc7c(ccc8ccccc87)c6)cc5)c5cc6ccccc6c6ccccc56)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4cc5ccccc5c5ccccc45)cc3)c3ccccc3c3ccccc23)cc1 ; c1ccc(-n2c3cc(-c4ccc(N(c5ccc6c(c5)C(c5ccccc5)(c5ccccc5)c5ccccc5-6)c5cc6ccccc6c6ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5cc6ccccc6c6ccccc56)cc4)ccc3c3ccc4ccccc4c32)cc1] 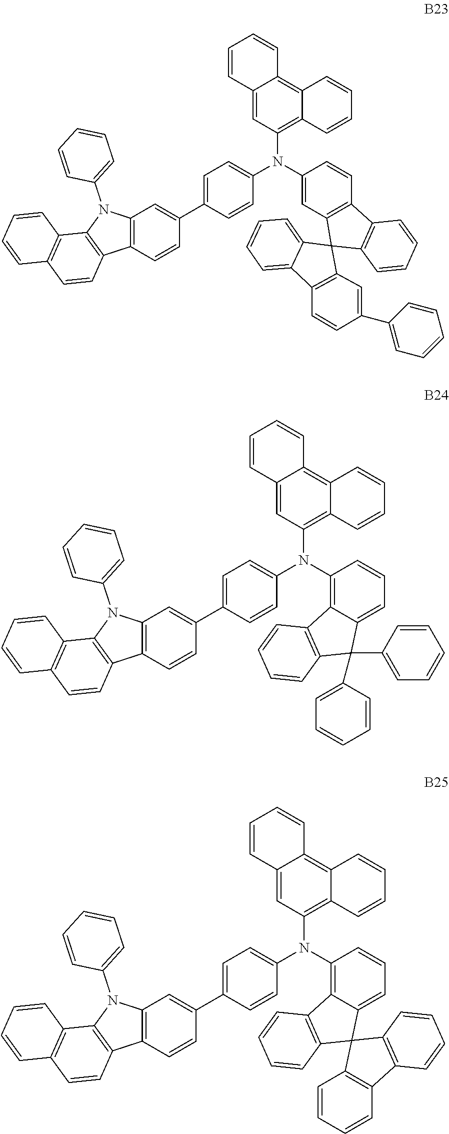 [SMILES: c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4cc5ccccc5c5ccccc45)cc32)cc1 ; c1ccc(-n2c3cc(-c4ccc(N(c5cccc6c5-c5ccccc5C6(c5ccccc5)c5ccccc5)c5cc6ccccc6c6ccccc56)cc4)ccc3c3ccc4ccccc4c32)cc1 ; c1ccc(-n2c3cc(-c4ccc(N(c5cccc6c5-c5ccccc5C65c6ccccc6-c6ccccc65)c5cc6ccccc6c6ccccc56)cc4)ccc3c3ccc4ccccc4c32)cc1] 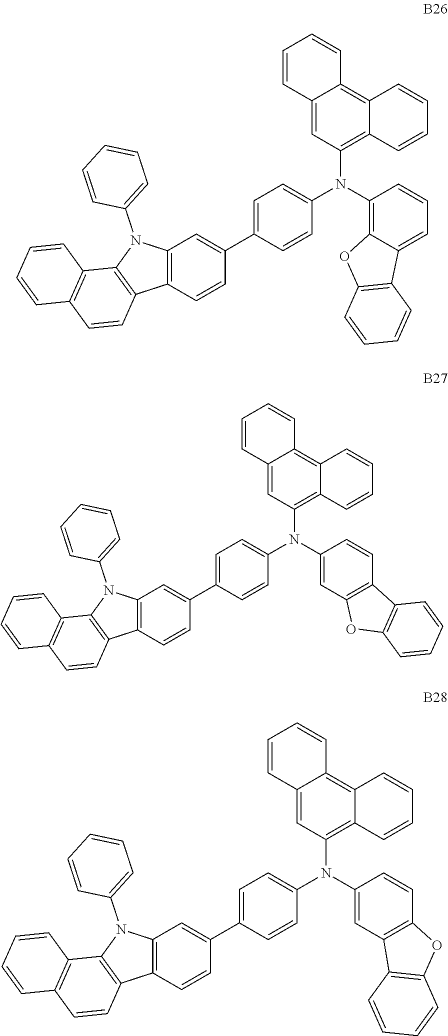 [SMILES: c1ccc(-n2c3cc(-c4ccc(N(c5cc6ccccc6c6ccccc56)c5cccc6c5oc5ccccc56)cc4)ccc3c3ccc4ccccc4c32)cc1 ; c1ccc(-n2c3cc(-c4ccc(N(c5ccc6c(c5)oc5ccccc56)c5cc6ccccc6c6ccccc56)cc4)ccc3c3ccc4ccccc4c32)cc1 ; c1ccc(-n2c3cc(-c4ccc(N(c5ccc6oc7ccccc7c6c5)c5cc6ccccc6c6ccccc56)cc4)ccc3c3ccc4ccccc4c32)cc1] 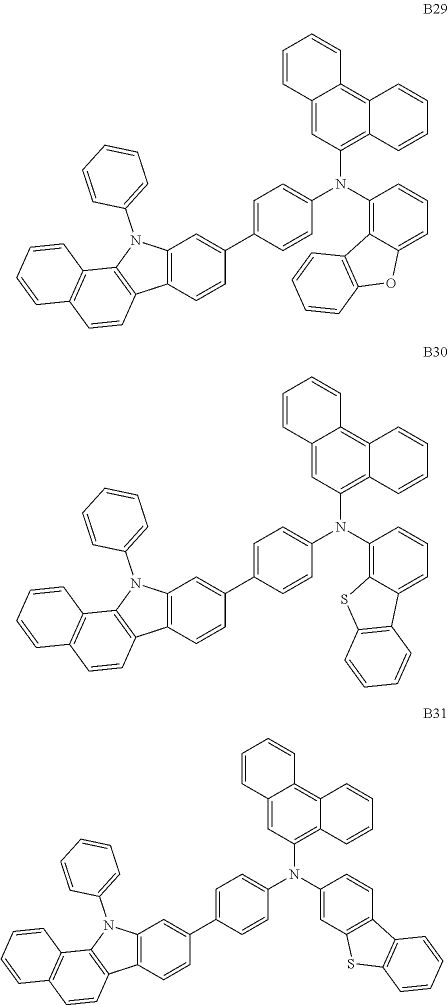 [SMILES: c1ccc(-n2c3cc(-c4ccc(N(c5cc6ccccc6c6ccccc56)c5cccc6oc7ccccc7c56)cc4)ccc3c3ccc4ccccc4c32)cc1 ; c1ccc(-n2c3cc(-c4ccc(N(c5cc6ccccc6c6ccccc56)c5cccc6c5sc5ccccc56)cc4)ccc3c3ccc4ccccc4c32)cc1 ; c1ccc(-n2c3cc(-c4ccc(N(c5ccc6c(c5)sc5ccccc56)c5cc6ccccc6c6ccccc56)cc4)ccc3c3ccc4ccccc4c32)cc1] 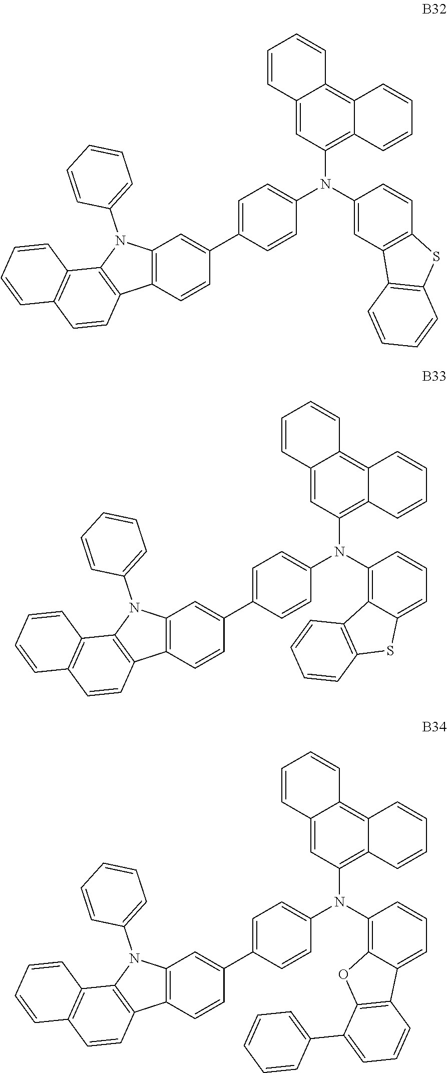 [SMILES: c1ccc(-n2c3cc(-c4ccc(N(c5ccc6sc7ccccc7c6c5)c5cc6ccccc6c6ccccc56)cc4)ccc3c3ccc4ccccc4c32)cc1 ; c1ccc(-n2c3cc(-c4ccc(N(c5cc6ccccc6c6ccccc56)c5cccc6sc7ccccc7c56)cc4)ccc3c3ccc4ccccc4c32)cc1 ; c1ccc(-c2cccc3c2oc2c(N(c4ccc(-c5ccc6c7ccc8ccccc8c7n(-c7ccccc7)c6c5)cc4)c4cc5ccccc5c5ccccc45)cccc23)cc1] 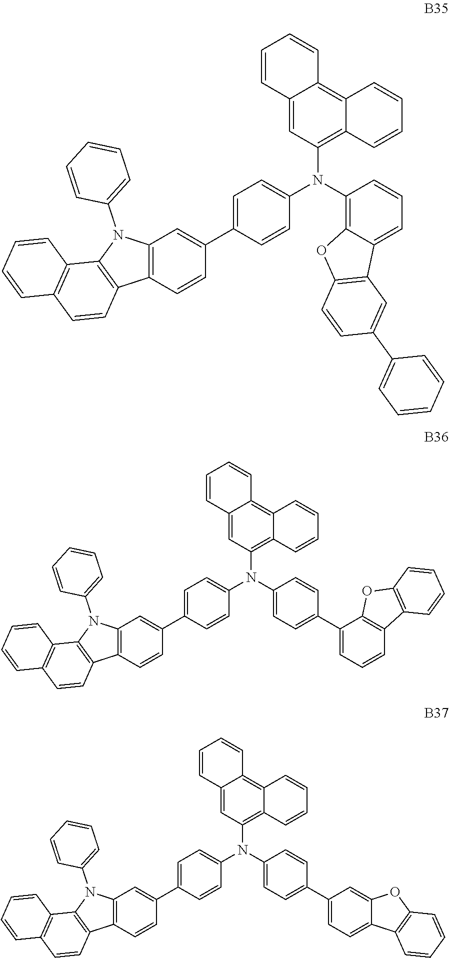 [SMILES: c1ccc(-c2ccc3oc4c(N(c5ccc(-c6ccc7c8ccc9ccccc9c8n(-c8ccccc8)c7c6)cc5)c5cc6ccccc6c6ccccc56)cccc4c3c2)cc1 ; c1ccc(-n2c3cc(-c4ccc(N(c5ccc(-c6cccc7c6oc6ccccc67)cc5)c5cc6ccccc6c6ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5cc6ccccc6c6ccccc56)cc4)ccc3c3ccc4ccccc4c32)cc1] 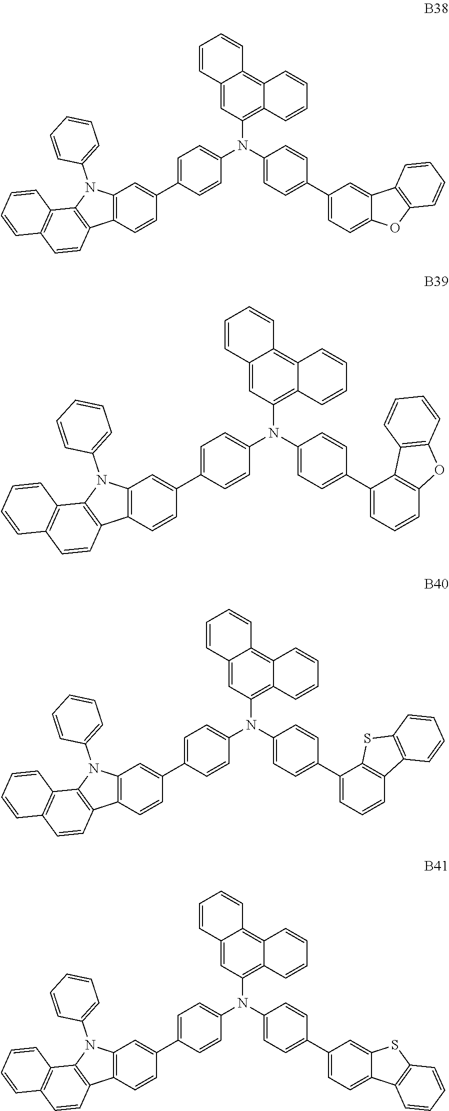 [SMILES: c1ccc(-n2c3cc(-c4ccc(N(c5ccc(-c6ccc7oc8ccccc8c7c6)cc5)c5cc6ccccc6c6ccccc56)cc4)ccc3c3ccc4ccccc4c32)cc1 ; c1ccc(-n2c3cc(-c4ccc(N(c5ccc(-c6cccc7oc8ccccc8c67)cc5)c5cc6ccccc6c6ccccc56)cc4)ccc3c3ccc4ccccc4c32)cc1 ; c1ccc(-n2c3cc(-c4ccc(N(c5ccc(-c6cccc7c6sc6ccccc67)cc5)c5cc6ccccc6c6ccccc56)cc4)ccc3c3ccc4ccccc4c32)cc1 ; c1ccc(-n2c3cc(-c4ccc(N(c5ccc(-c6ccc7c(c6)sc6ccccc67)cc5)c5cc6ccccc6c6ccccc56)cc4)ccc3c3ccc4ccccc4c32)cc1] 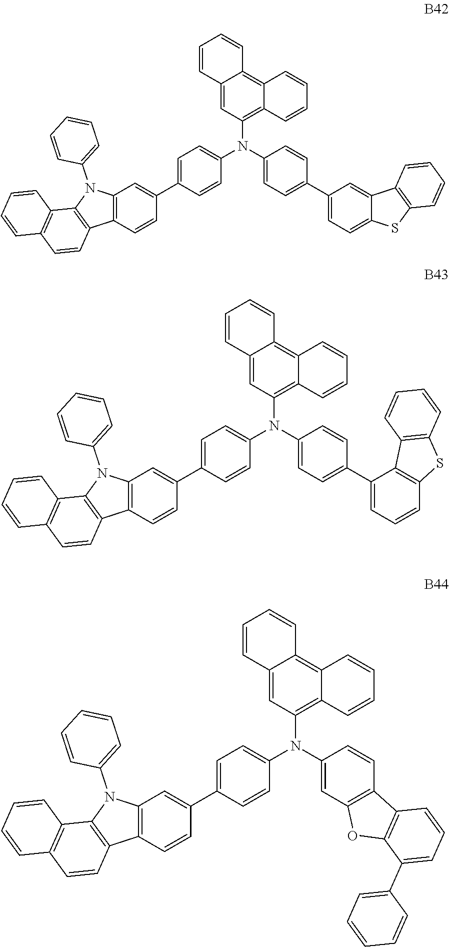 [SMILES: c1ccc(-n2c3cc(-c4ccc(N(c5ccc(-c6ccc7sc8ccccc8c7c6)cc5)c5cc6ccccc6c6ccccc56)cc4)ccc3c3ccc4ccccc4c32)cc1 ; c1ccc(-n2c3cc(-c4ccc(N(c5ccc(-c6cccc7sc8ccccc8c67)cc5)c5cc6ccccc6c6ccccc56)cc4)ccc3c3ccc4ccccc4c32)cc1 ; c1ccc(-c2cccc3c2oc2cc(N(c4ccc(-c5ccc6c7ccc8ccccc8c7n(-c7ccccc7)c6c5)cc4)c4cc5ccccc5c5ccccc45)ccc23)cc1] 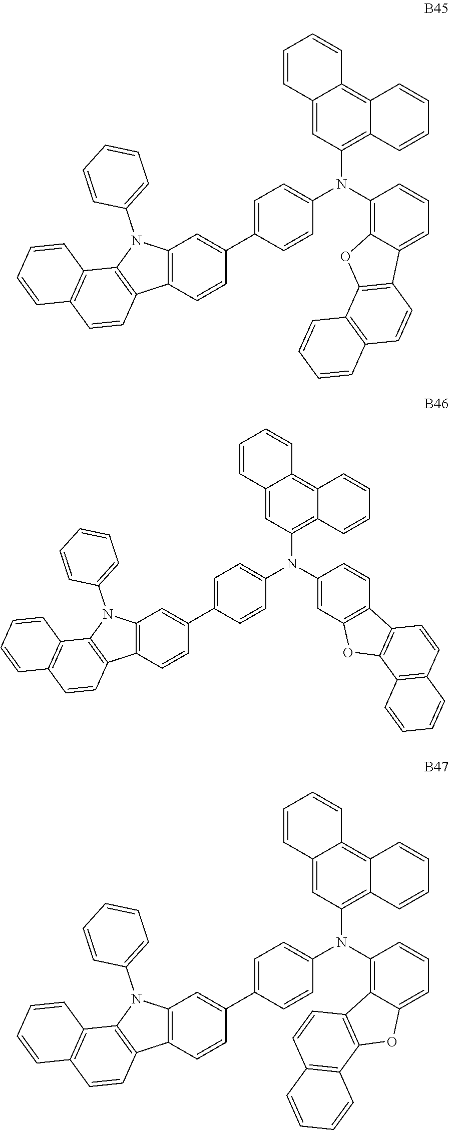 [SMILES: c1ccc(-n2c3cc(-c4ccc(N(c5cc6ccccc6c6ccccc56)c5cccc6c5oc5c7ccccc7ccc65)cc4)ccc3c3ccc4ccccc4c32)cc1 ; c1ccc(-n2c3cc(-c4ccc(N(c5ccc6c(c5)oc5c7ccccc7ccc65)c5cc6ccccc6c6ccccc56)cc4)ccc3c3ccc4ccccc4c32)cc1 ; c1ccc(-n2c3cc(-c4ccc(N(c5cc6ccccc6c6ccccc56)c5cccc6oc7c8ccccc8ccc7c56)cc4)ccc3c3ccc4ccccc4c32)cc1] 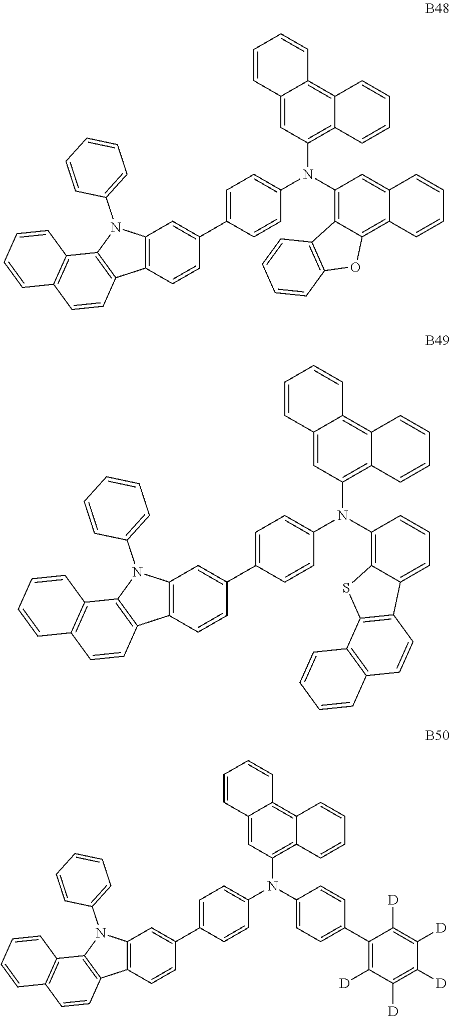 [SMILES: c1ccc(-n2c3cc(-c4ccc(N(c5cc6ccccc6c6ccccc56)c5cc6ccccc6c6oc7ccccc7c56)cc4)ccc3c3ccc4ccccc4c32)cc1 ; c1ccc(-n2c3cc(-c4ccc(N(c5cc6ccccc6c6ccccc56)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3cc4ccccc4c4ccccc34)cc2)c([2H])c1[2H]] 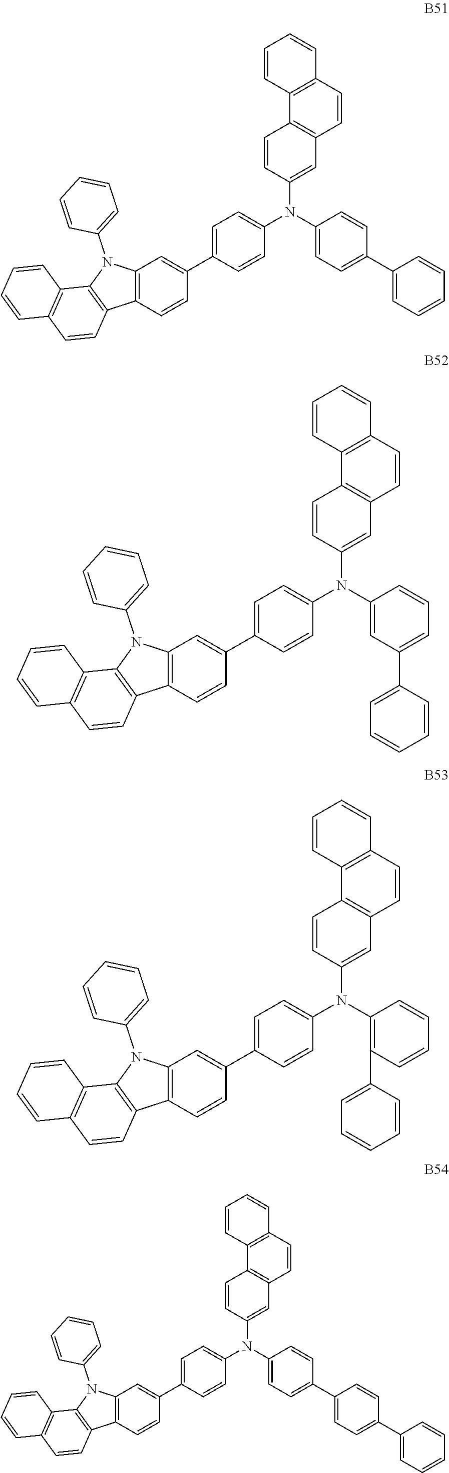 [SMILES: c1ccc(-c2ccc(N(c3ccc(-c4ccc5c6ccc7ccccc7c6n(-c6ccccc6)c5c4)cc3)c3ccc4c(ccc5ccccc54)c3)cc2)cc1 ; c1ccc(-c2cccc(N(c3ccc(-c4ccc5c6ccc7ccccc7c6n(-c6ccccc6)c5c4)cc3)c3ccc4c(ccc5ccccc54)c3)c2)cc1 ; c1ccc(-c2ccccc2N(c2ccc(-c3ccc4c5ccc6ccccc6c5n(-c5ccccc5)c4c3)cc2)c2ccc3c(ccc4ccccc43)c2)cc1 ; c1ccc(-c2ccc(-c3ccc(N(c4ccc(-c5ccc6c7ccc8ccccc8c7n(-c7ccccc7)c6c5)cc4)c4ccc5c(ccc6ccccc65)c4)cc3)cc2)cc1] 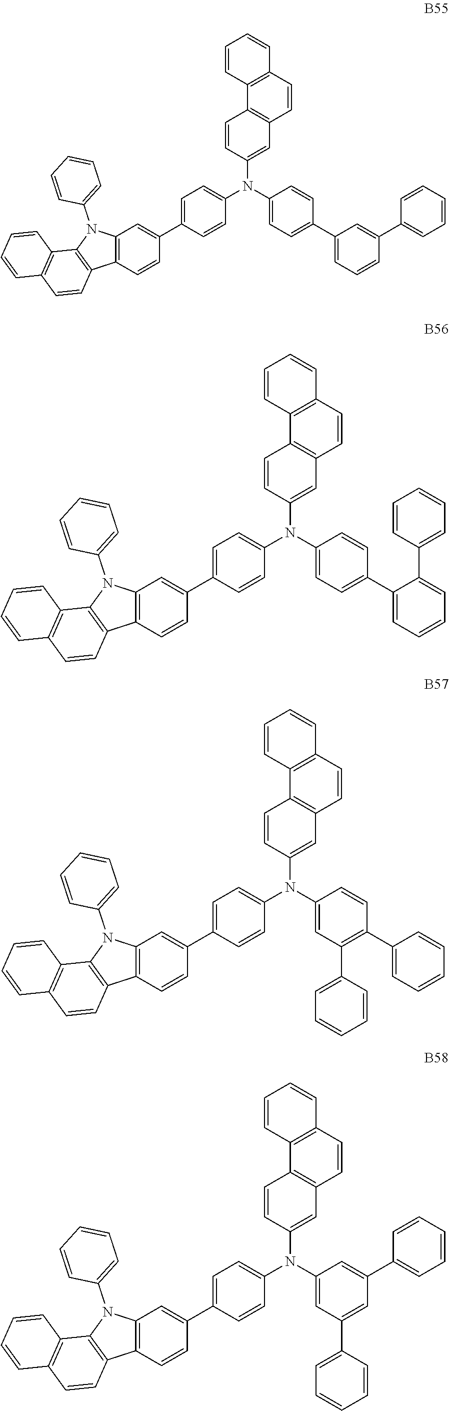 [SMILES: c1ccc(-c2cccc(-c3ccc(N(c4ccc(-c5ccc6c7ccc8ccccc8c7n(-c7ccccc7)c6c5)cc4)c4ccc5c(ccc6ccccc65)c4)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(ccc5ccccc54)c3)cc2)cc1 ; c1ccc(-c2ccc(N(c3ccc(-c4ccc5c6ccc7ccccc7c6n(-c6ccccc6)c5c4)cc3)c3ccc4c(ccc5ccccc54)c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(ccc5ccccc54)c3)c2)cc1] 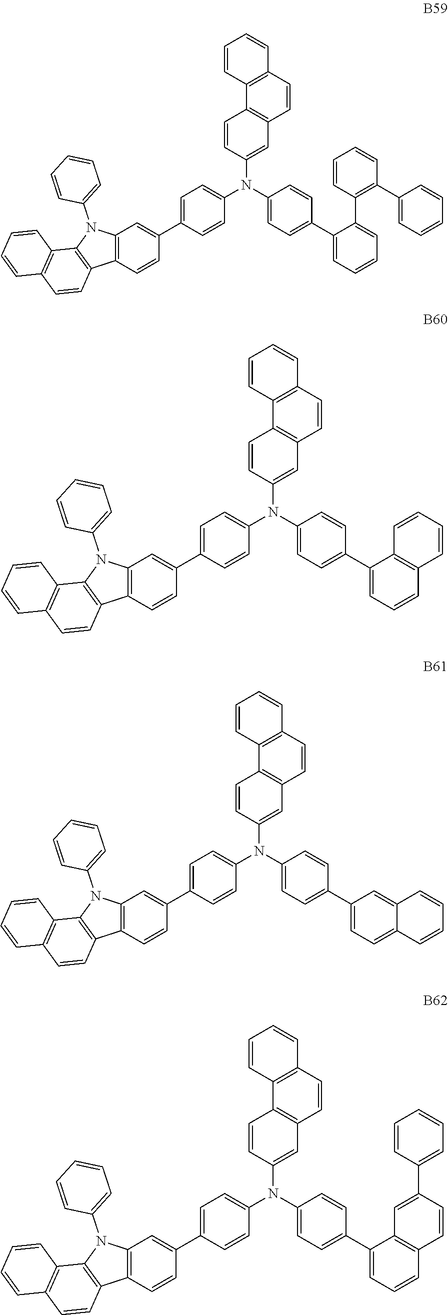 [SMILES: c1ccc(-c2ccccc2-c2ccccc2-c2ccc(N(c3ccc(-c4ccc5c6ccc7ccccc7c6n(-c6ccccc6)c5c4)cc3)c3ccc4c(ccc5ccccc54)c3)cc2)cc1 ; c1ccc(-n2c3cc(-c4ccc(N(c5ccc(-c6cccc7ccccc67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7ccccc7c6)cc5)c5ccc6c(ccc7ccccc76)c5)cc4)ccc3c3ccc4ccccc4c32)cc1 ; c1ccc(-c2ccc3cccc(-c4ccc(N(c5ccc(-c6ccc7c8ccc9ccccc9c8n(-c8ccccc8)c7c6)cc5)c5ccc6c(ccc7ccccc76)c5)cc4)c3c2)cc1] 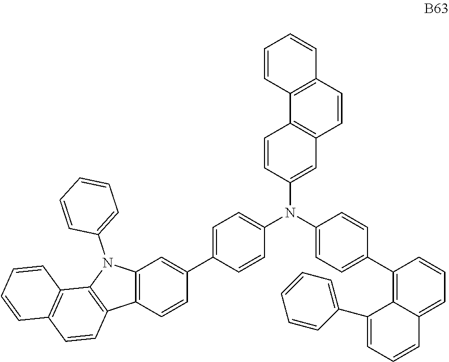 [SMILES: c1ccc(-c2cccc3cccc(-c4ccc(N(c5ccc(-c6ccc7c8ccc9ccccc9c8n(-c8ccccc8)c7c6)cc5)c5ccc6c(ccc7ccccc76)c5)cc4)c23)cc1] 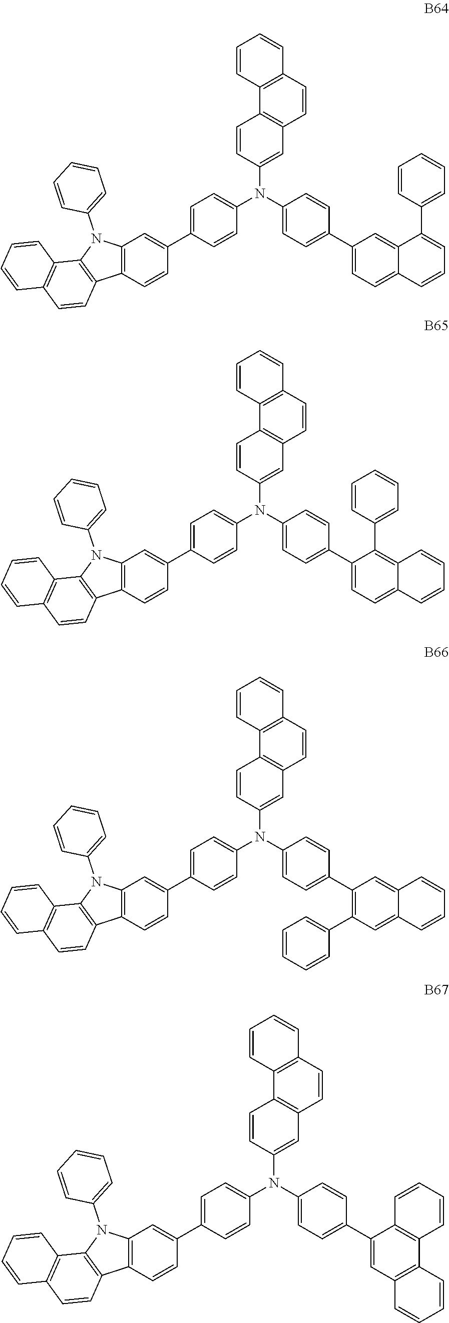 [SMILES: c1ccc(-c2cccc3ccc(-c4ccc(N(c5ccc(-c6ccc7c8ccc9ccccc9c8n(-c8ccccc8)c7c6)cc5)c5ccc6c(ccc7ccccc76)c5)cc4)cc23)cc1 ; c1ccc(-c2c(-c3ccc(N(c4ccc(-c5ccc6c7ccc8ccccc8c7n(-c7ccccc7)c6c5)cc4)c4ccc5c(ccc6ccccc65)c4)cc3)ccc3ccccc23)cc1 ; c1ccc(-c2cc3ccccc3cc2-c2ccc(N(c3ccc(-c4ccc5c6ccc7ccccc7c6n(-c6ccccc6)c5c4)cc3)c3ccc4c(ccc5ccccc54)c3)cc2)cc1 ; c1ccc(-n2c3cc(-c4ccc(N(c5ccc(-c6cc7ccccc7c7ccccc67)cc5)c5ccc6c(ccc7ccccc76)c5)cc4)ccc3c3ccc4ccccc4c32)cc1] 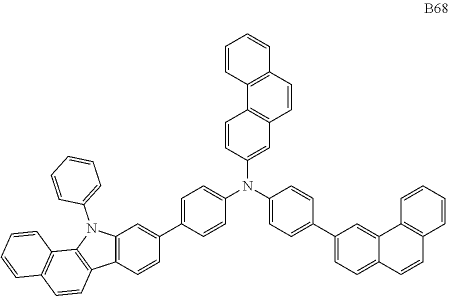 [SMILES: c1ccc(-n2c3cc(-c4ccc(N(c5ccc(-c6ccc7ccc8ccccc8c7c6)cc5)c5ccc6c(ccc7ccccc76)c5)cc4)ccc3c3ccc4ccccc4c32)cc1] 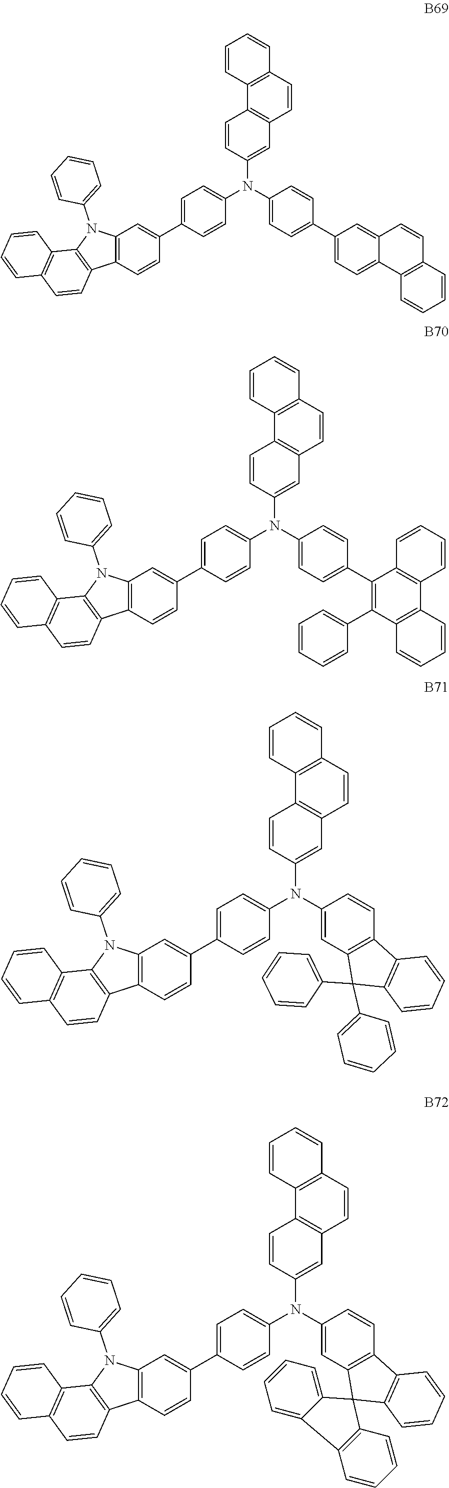 [SMILES: c1ccc(-n2c3cc(-c4ccc(N(c5ccc(-c6ccc7c(ccc8ccccc87)c6)cc5)c5ccc6c(ccc7ccccc76)c5)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4ccc5c(ccc6ccccc65)c4)cc3)c3ccccc3c3ccccc23)cc1 ; c1ccc(-n2c3cc(-c4ccc(N(c5ccc6c(c5)C(c5ccccc5)(c5ccccc5)c5ccccc5-6)c5ccc6c(ccc7ccccc76)c5)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6c(ccc7ccccc76)c5)cc4)ccc3c3ccc4ccccc4c32)cc1] 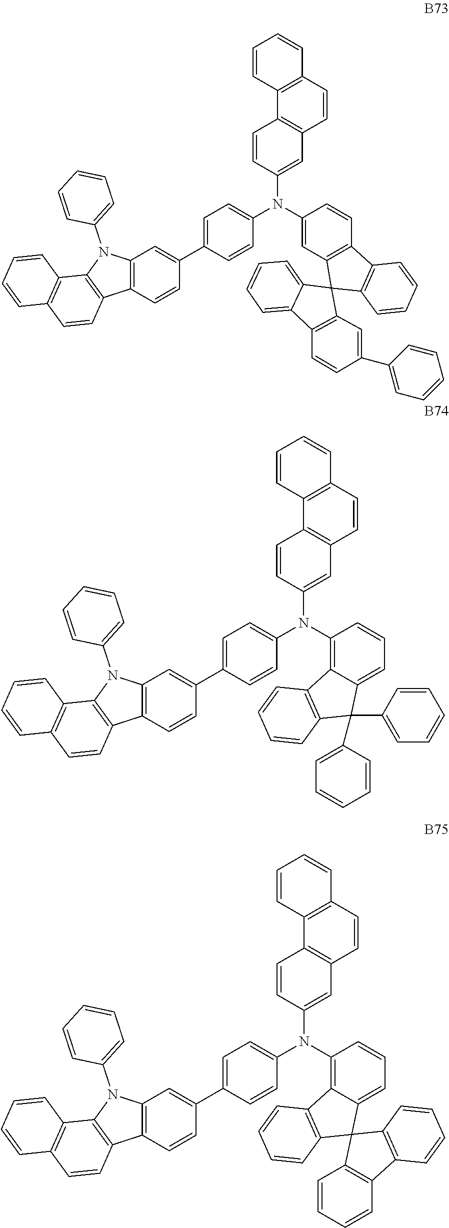 [SMILES: c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4ccc5c(ccc6ccccc65)c4)cc32)cc1 ; c1ccc(-n2c3cc(-c4ccc(N(c5ccc6c(ccc7ccccc76)c5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc6c(ccc7ccccc76)c5)c5cccc6c5-c5ccccc5C65c6ccccc6-c6ccccc65)cc4)ccc3c3ccc4ccccc4c32)cc1] 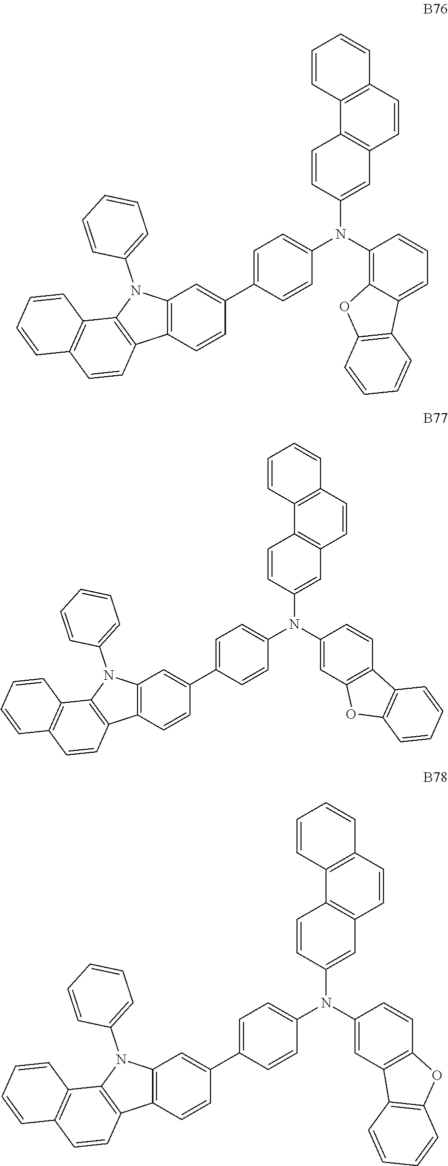 [SMILES: c1ccc(-n2c3cc(-c4ccc(N(c5ccc6c(ccc7ccccc76)c5)c5cccc6c5oc5ccccc56)cc4)ccc3c3ccc4ccccc4c32)cc1 ; c1ccc(-n2c3cc(-c4ccc(N(c5ccc6c(ccc7ccccc76)c5)c5ccc6c(c5)oc5ccccc56)cc4)ccc3c3ccc4ccccc4c32)cc1 ; c1ccc(-n2c3cc(-c4ccc(N(c5ccc6c(ccc7ccccc76)c5)c5ccc6oc7ccccc7c6c5)cc4)ccc3c3ccc4ccccc4c32)cc1] 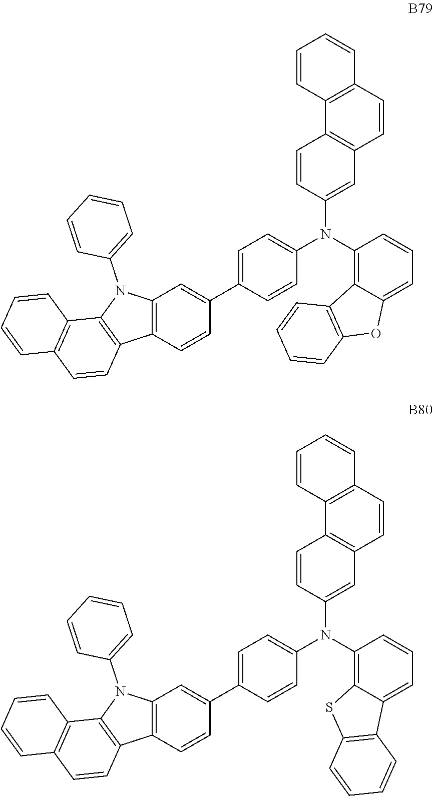 [SMILES: c1ccc(-n2c3cc(-c4ccc(N(c5ccc6c(ccc7ccccc76)c5)c5cccc6oc7ccccc7c56)cc4)ccc3c3ccc4ccccc4c32)cc1 ; c1ccc(-n2c3cc(-c4ccc(N(c5ccc6c(ccc7ccccc76)c5)c5cccc6c5sc5ccccc56)cc4)ccc3c3ccc4ccccc4c32)cc1] 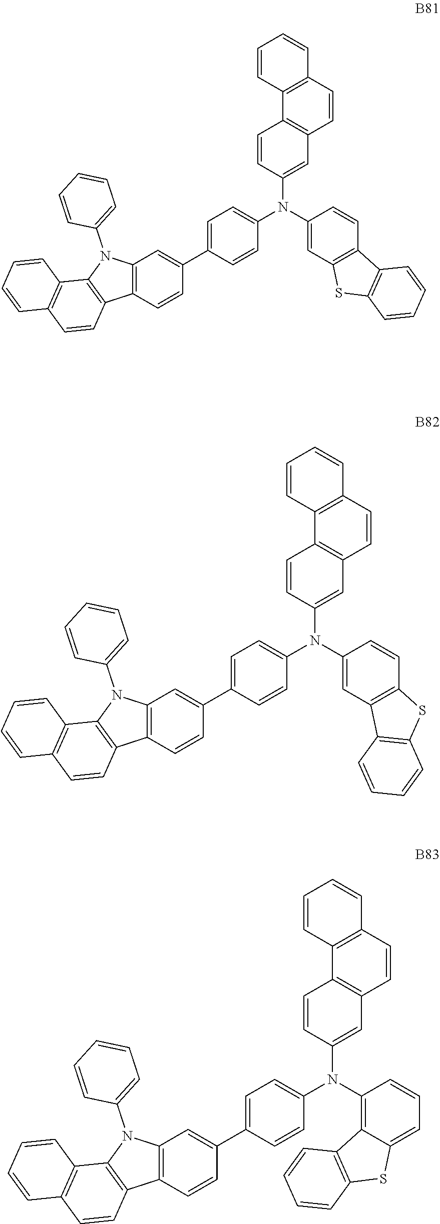 [SMILES: c1ccc(-n2c3cc(-c4ccc(N(c5ccc6c(ccc7ccccc76)c5)c5ccc6c(c5)sc5ccccc56)cc4)ccc3c3ccc4ccccc4c32)cc1 ; c1ccc(-n2c3cc(-c4ccc(N(c5ccc6c(ccc7ccccc76)c5)c5ccc6sc7ccccc7c6c5)cc4)ccc3c3ccc4ccccc4c32)cc1 ; c1ccc(-n2c3cc(-c4ccc(N(c5ccc6c(ccc7ccccc76)c5)c5cccc6sc7ccccc7c56)cc4)ccc3c3ccc4ccccc4c32)cc1] 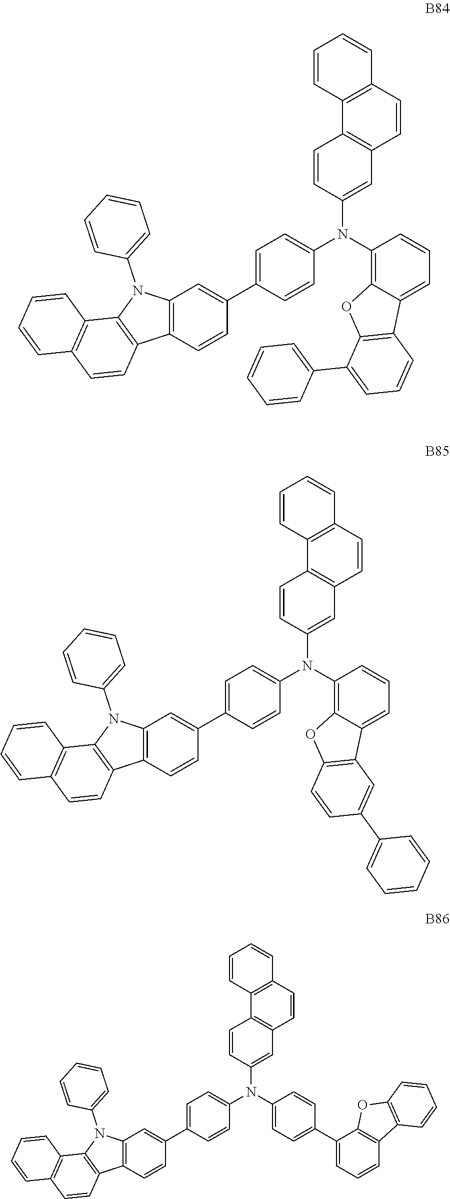 [SMILES: c1ccc(-c2cccc3c2oc2c(N(c4ccc(-c5ccc6c7ccc8ccccc8c7n(-c7ccccc7)c6c5)cc4)c4ccc5c(ccc6ccccc65)c4)cccc23)cc1 ; c1ccc(-c2ccc3oc4c(N(c5ccc(-c6ccc7c8ccc9ccccc9c8n(-c8ccccc8)c7c6)cc5)c5ccc6c(ccc7ccccc76)c5)cccc4c3c2)cc1 ; c1ccc(-n2c3cc(-c4ccc(N(c5ccc(-c6cccc7c6oc6ccccc67)cc5)c5ccc6c(ccc7ccccc76)c5)cc4)ccc3c3ccc4ccccc4c32)cc1] 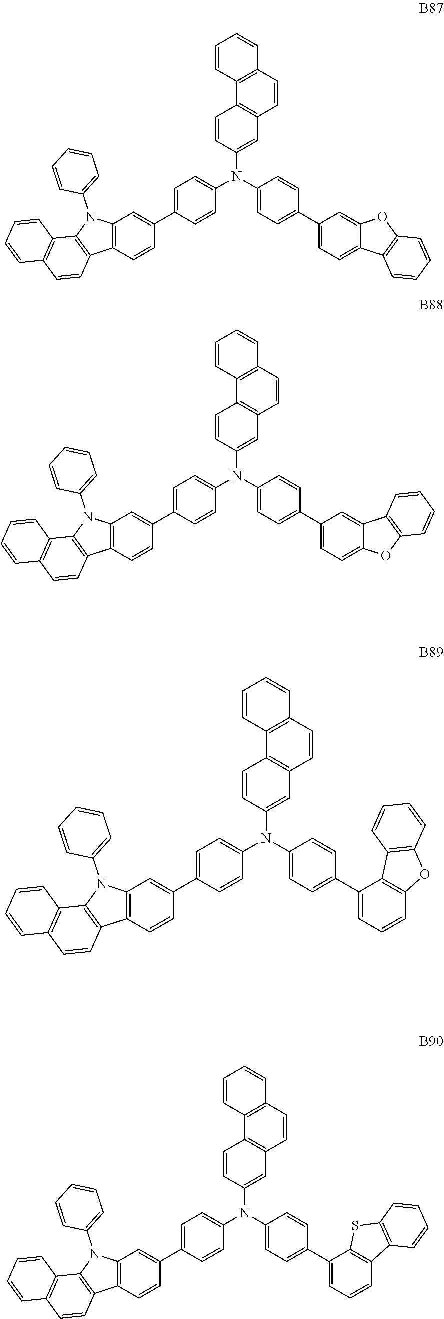 [SMILES: c1ccc(-n2c3cc(-c4ccc(N(c5ccc(-c6ccc7c(c6)oc6ccccc67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7oc8ccccc8c7c6)cc5)c5ccc6c(ccc7ccccc76)c5)cc4)ccc3c3ccc4ccccc4c32)cc1 ; c1ccc(-n2c3cc(-c4ccc(N(c5ccc(-c6cccc7oc8ccccc8c67)cc5)c5ccc6c(ccc7ccccc76)c5)cc4)ccc3c3ccc4ccccc4c32)cc1 ; c1ccc(-n2c3cc(-c4ccc(N(c5ccc(-c6cccc7c6sc6ccccc67)cc5)c5ccc6c(ccc7ccccc76)c5)cc4)ccc3c3ccc4ccccc4c32)cc1] 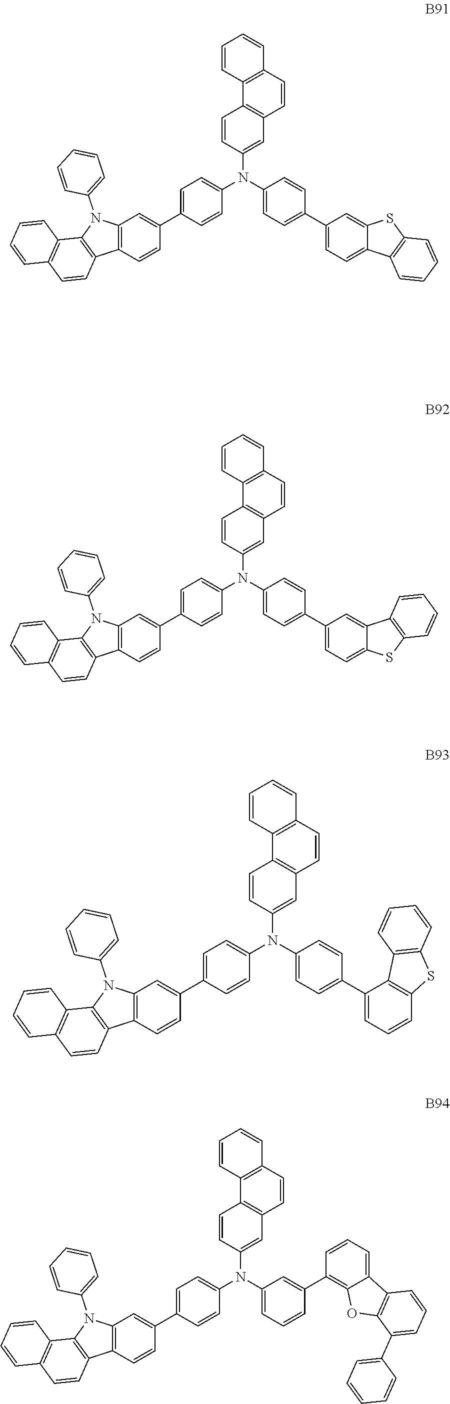 [SMILES: c1ccc(-n2c3cc(-c4ccc(N(c5ccc(-c6ccc7c(c6)sc6ccccc67)cc5)c5ccc6c(ccc7ccccc76)c5)cc4)ccc3c3ccc4ccccc4c32)cc1 ; c1ccc(-n2c3cc(-c4ccc(N(c5ccc(-c6ccc7sc8ccccc8c7c6)cc5)c5ccc6c(ccc7ccccc76)c5)cc4)ccc3c3ccc4ccccc4c32)cc1 ; c1ccc(-n2c3cc(-c4ccc(N(c5ccc(-c6cccc7sc8ccccc8c67)cc5)c5ccc6c(ccc7ccccc76)c5)cc4)ccc3c3ccc4ccccc4c32)cc1 ; c1ccc(-c2cccc3c2oc2c(-c4cccc(N(c5ccc(-c6ccc7c8ccc9ccccc9c8n(-c8ccccc8)c7c6)cc5)c5ccc6c(ccc7ccccc76)c5)c4)cccc23)cc1] 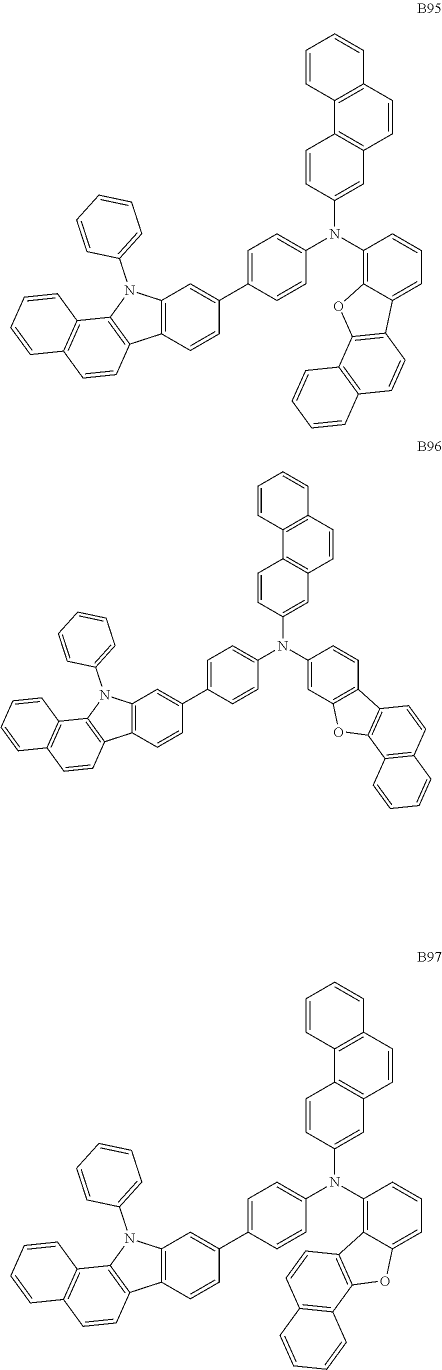 [SMILES: c1ccc(-n2c3cc(-c4ccc(N(c5ccc6c(ccc7ccccc76)c5)c5cccc6c5oc5c7ccccc7ccc65)cc4)ccc3c3ccc4ccccc4c32)cc1 ; c1ccc(-n2c3cc(-c4ccc(N(c5ccc6c(ccc7ccccc76)c5)c5ccc6c(c5)oc5c7ccccc7ccc65)cc4)ccc3c3ccc4ccccc4c32)cc1 ; c1ccc(-n2c3cc(-c4ccc(N(c5ccc6c(ccc7ccccc76)c5)c5cccc6oc7c8ccccc8ccc7c56)cc4)ccc3c3ccc4ccccc4c32)cc1] 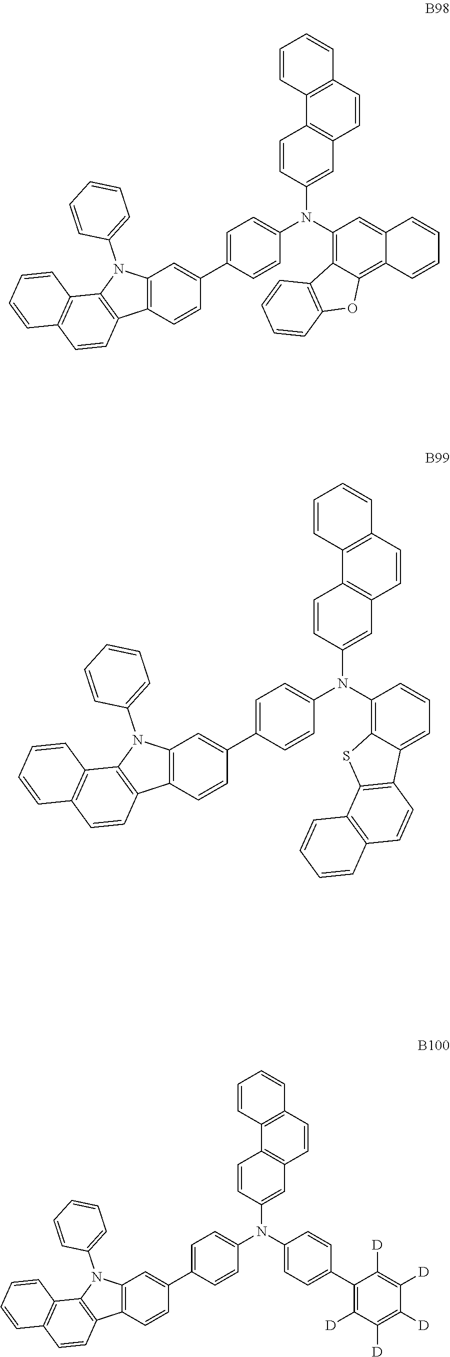 [SMILES: c1ccc(-n2c3cc(-c4ccc(N(c5ccc6c(ccc7ccccc76)c5)c5cc6ccccc6c6oc7ccccc7c56)cc4)ccc3c3ccc4ccccc4c32)cc1 ; c1ccc(-n2c3cc(-c4ccc(N(c5ccc6c(ccc7ccccc76)c5)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4c(ccc5ccccc54)c3)cc2)c([2H])c1[2H]] 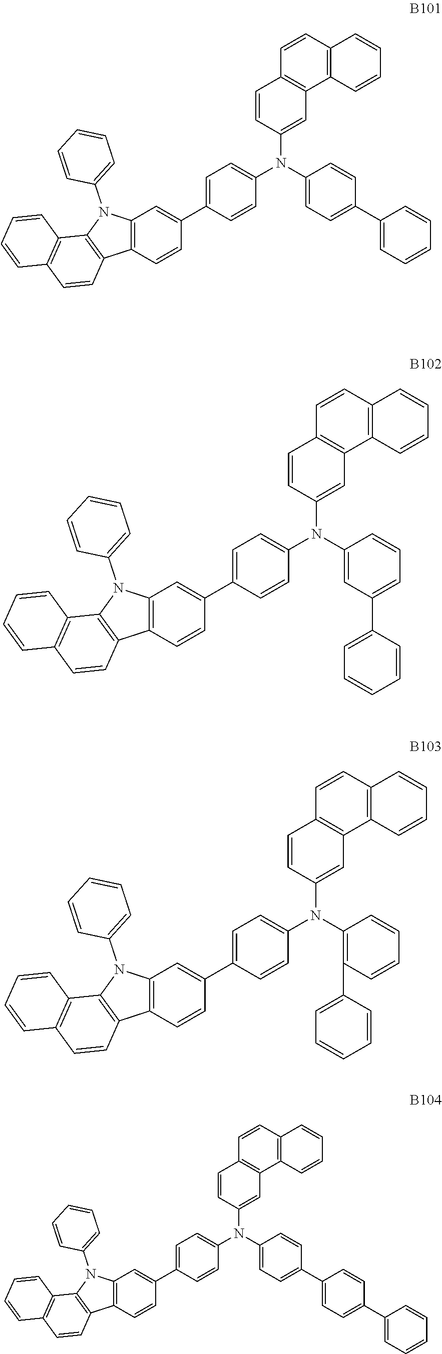 [SMILES: c1ccc(-c2ccc(N(c3ccc(-c4ccc5c6ccc7ccccc7c6n(-c6ccccc6)c5c4)cc3)c3ccc4ccc5ccccc5c4c3)cc2)cc1 ; c1ccc(-c2cccc(N(c3ccc(-c4ccc5c6ccc7ccccc7c6n(-c6ccccc6)c5c4)cc3)c3ccc4ccc5ccccc5c4c3)c2)cc1 ; c1ccc(-c2ccccc2N(c2ccc(-c3ccc4c5ccc6ccccc6c5n(-c5ccccc5)c4c3)cc2)c2ccc3ccc4ccccc4c3c2)cc1 ; c1ccc(-c2ccc(-c3ccc(N(c4ccc(-c5ccc6c7ccc8ccccc8c7n(-c7ccccc7)c6c5)cc4)c4ccc5ccc6ccccc6c5c4)cc3)cc2)cc1] 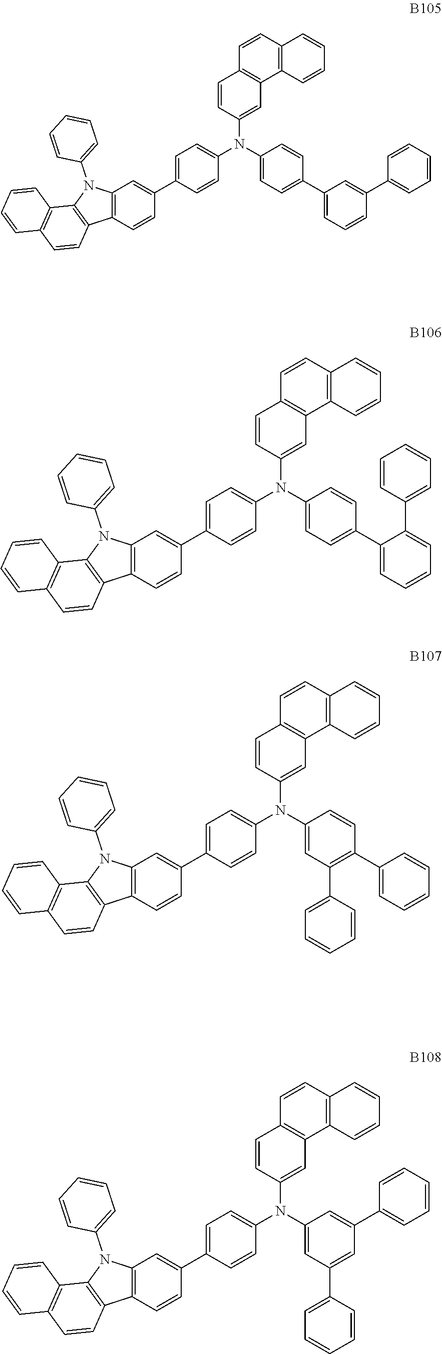 [SMILES: c1ccc(-c2cccc(-c3ccc(N(c4ccc(-c5ccc6c7ccc8ccccc8c7n(-c7ccccc7)c6c5)cc4)c4ccc5ccc6ccccc6c5c4)cc3)c2)cc1 ; c1ccc(-c2ccccc2-c2ccc(N(c3ccc(-c4ccc5c6ccc7ccccc7c6n(-c6ccccc6)c5c4)cc3)c3ccc4ccc5ccccc5c4c3)cc2)cc1 ; c1ccc(-c2ccc(N(c3ccc(-c4ccc5c6ccc7ccccc7c6n(-c6ccccc6)c5c4)cc3)c3ccc4ccc5ccccc5c4c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4ccc5ccccc5c4c3)c2)cc1] 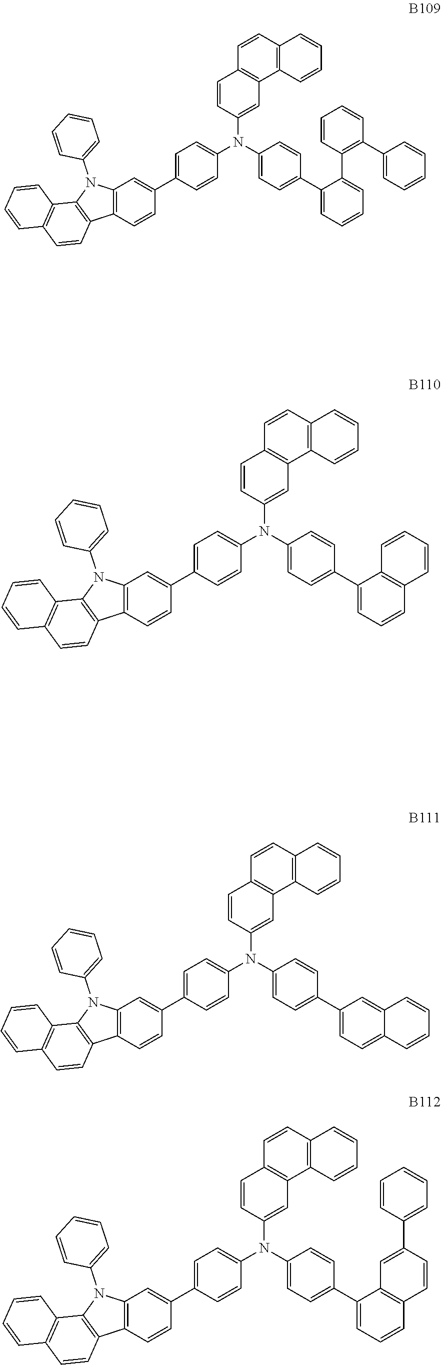 [SMILES: c1ccc(-c2ccccc2-c2ccccc2-c2ccc(N(c3ccc(-c4ccc5c6ccc7ccccc7c6n(-c6ccccc6)c5c4)cc3)c3ccc4ccc5ccccc5c4c3)cc2)cc1 ; c1ccc(-n2c3cc(-c4ccc(N(c5ccc(-c6cccc7ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7ccccc7c6)cc5)c5ccc6ccc7ccccc7c6c5)cc4)ccc3c3ccc4ccccc4c32)cc1 ; c1ccc(-c2ccc3cccc(-c4ccc(N(c5ccc(-c6ccc7c8ccc9ccccc9c8n(-c8ccccc8)c7c6)cc5)c5ccc6ccc7ccccc7c6c5)cc4)c3c2)cc1] 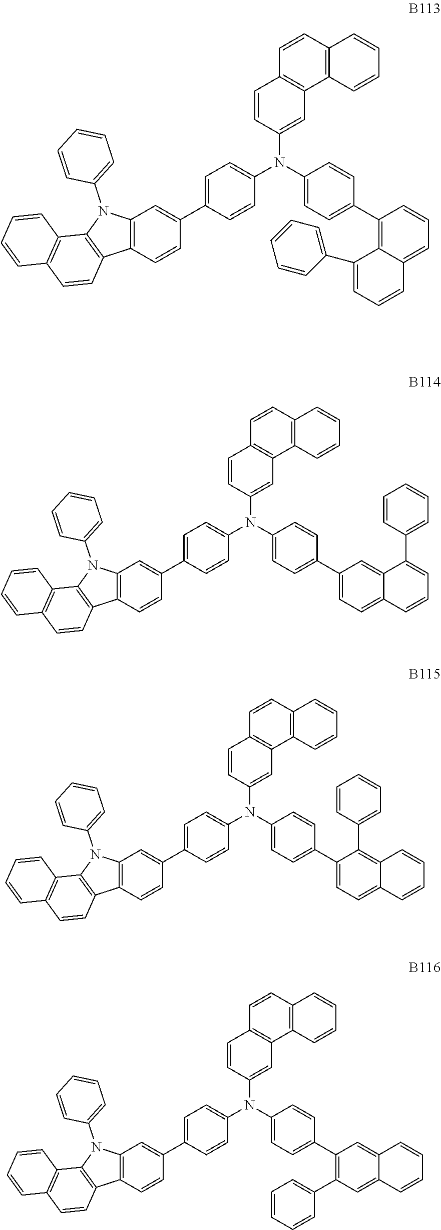 [SMILES: c1ccc(-c2cccc3cccc(-c4ccc(N(c5ccc(-c6ccc7c8ccc9ccccc9c8n(-c8ccccc8)c7c6)cc5)c5ccc6ccc7ccccc7c6c5)cc4)c23)cc1 ; c1ccc(-c2cccc3ccc(-c4ccc(N(c5ccc(-c6ccc7c8ccc9ccccc9c8n(-c8ccccc8)c7c6)cc5)c5ccc6ccc7ccccc7c6c5)cc4)cc23)cc1 ; c1ccc(-c2c(-c3ccc(N(c4ccc(-c5ccc6c7ccc8ccccc8c7n(-c7ccccc7)c6c5)cc4)c4ccc5ccc6ccccc6c5c4)cc3)ccc3ccccc23)cc1 ; c1ccc(-c2cc3ccccc3cc2-c2ccc(N(c3ccc(-c4ccc5c6ccc7ccccc7c6n(-c6ccccc6)c5c4)cc3)c3ccc4ccc5ccccc5c4c3)cc2)cc1] 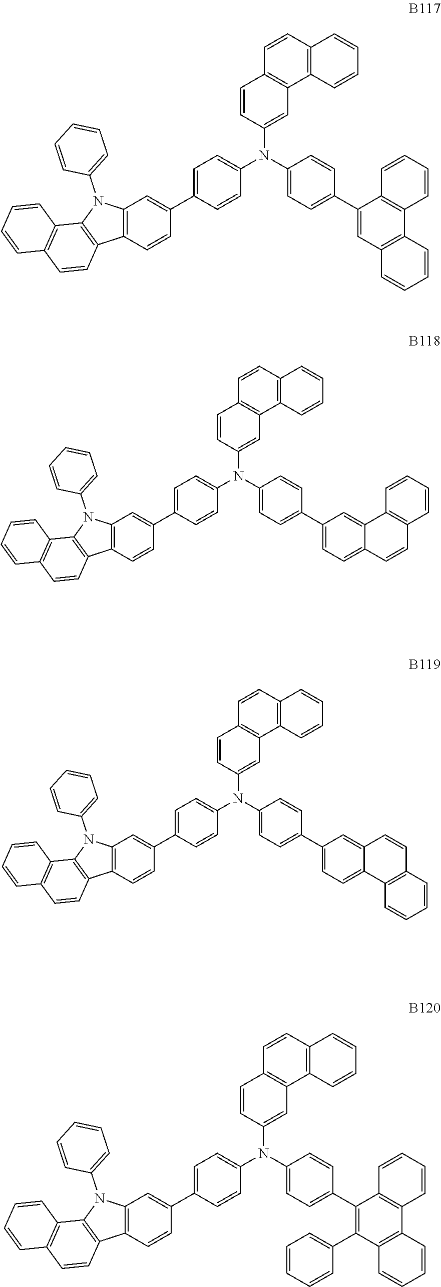 [SMILES: c1ccc(-n2c3cc(-c4ccc(N(c5ccc(-c6cc7ccccc7c7ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7ccc8ccccc8c7c6)cc5)c5ccc6ccc7ccccc7c6c5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6ccc7ccccc7c6c5)cc4)ccc3c3ccc4ccccc4c32)cc1 ; c1ccc(-c2c(-c3ccc(N(c4ccc(-c5ccc6c7ccc8ccccc8c7n(-c7ccccc7)c6c5)cc4)c4ccc5ccc6ccccc6c5c4)cc3)c3ccccc3c3ccccc23)cc1] 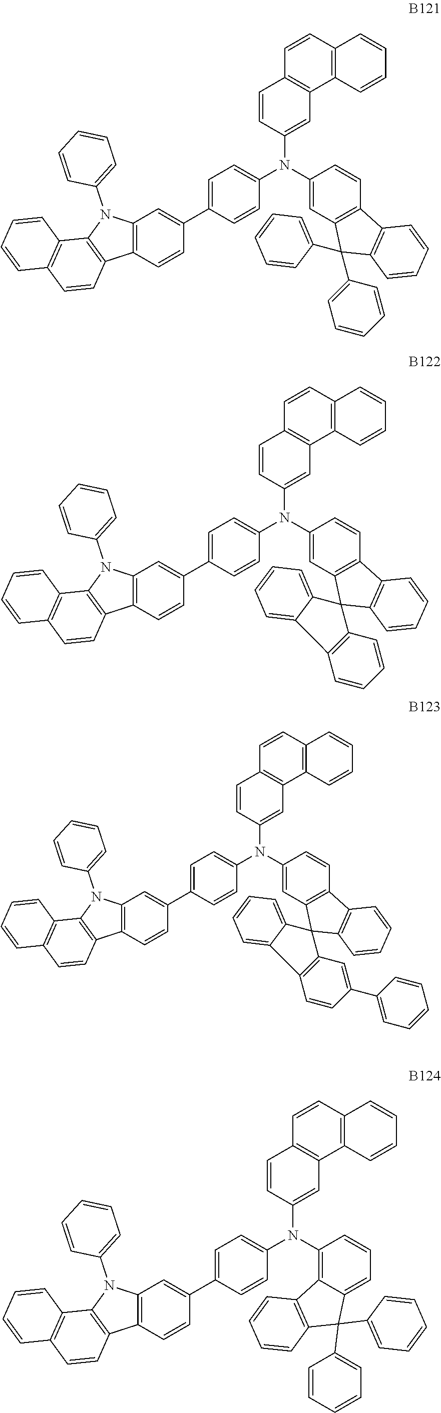 [SMILES: c1ccc(-n2c3cc(-c4ccc(N(c5ccc6c(c5)C(c5ccccc5)(c5ccccc5)c5ccccc5-6)c5ccc6ccc7ccccc7c6c5)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6ccc7ccccc7c6c5)cc4)ccc3c3ccc4ccccc4c32)cc1 ; c1ccc(-c2ccc3c(c2)C2(c4ccccc4-3)c3ccccc3-c3ccc(N(c4ccc(-c5ccc6c7ccc8ccccc8c7n(-c7ccccc7)c6c5)cc4)c4ccc5ccc6ccccc6c5c4)cc32)cc1 ; c1ccc(-n2c3cc(-c4ccc(N(c5ccc6ccc7ccccc7c6c5)c5cccc6c5-c5ccccc5C6(c5ccccc5)c5ccccc5)cc4)ccc3c3ccc4ccccc4c32)cc1] 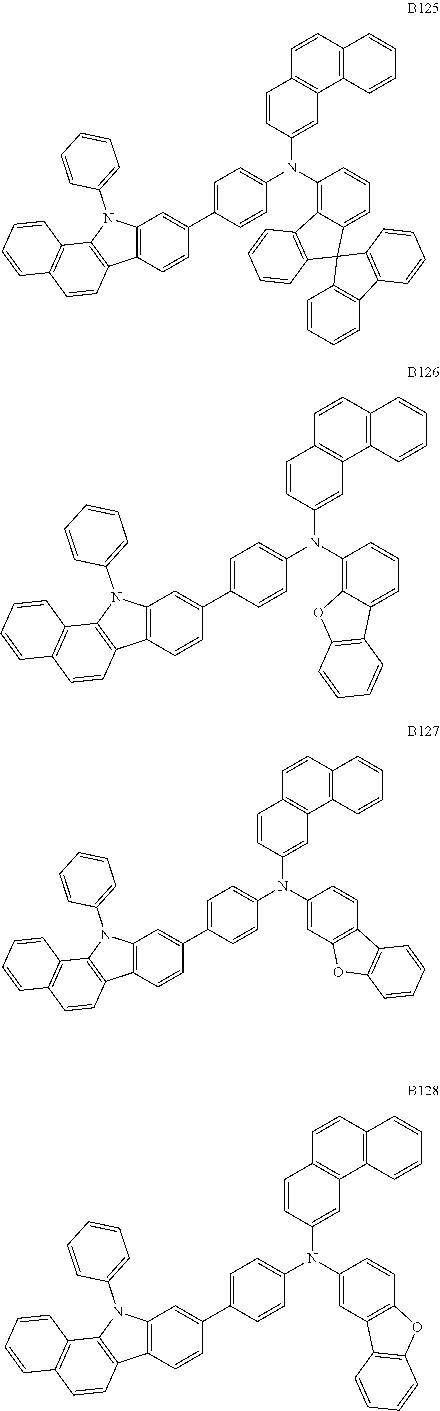 [SMILES: c1ccc(-n2c3cc(-c4ccc(N(c5ccc6ccc7ccccc7c6c5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc6ccc7ccccc7c6c5)c5cccc6c5oc5ccccc56)cc4)ccc3c3ccc4ccccc4c32)cc1 ; c1ccc(-n2c3cc(-c4ccc(N(c5ccc6c(c5)oc5ccccc56)c5ccc6ccc7ccccc7c6c5)cc4)ccc3c3ccc4ccccc4c32)cc1 ; c1ccc(-n2c3cc(-c4ccc(N(c5ccc6ccc7ccccc7c6c5)c5ccc6oc7ccccc7c6c5)cc4)ccc3c3ccc4ccccc4c32)cc1] 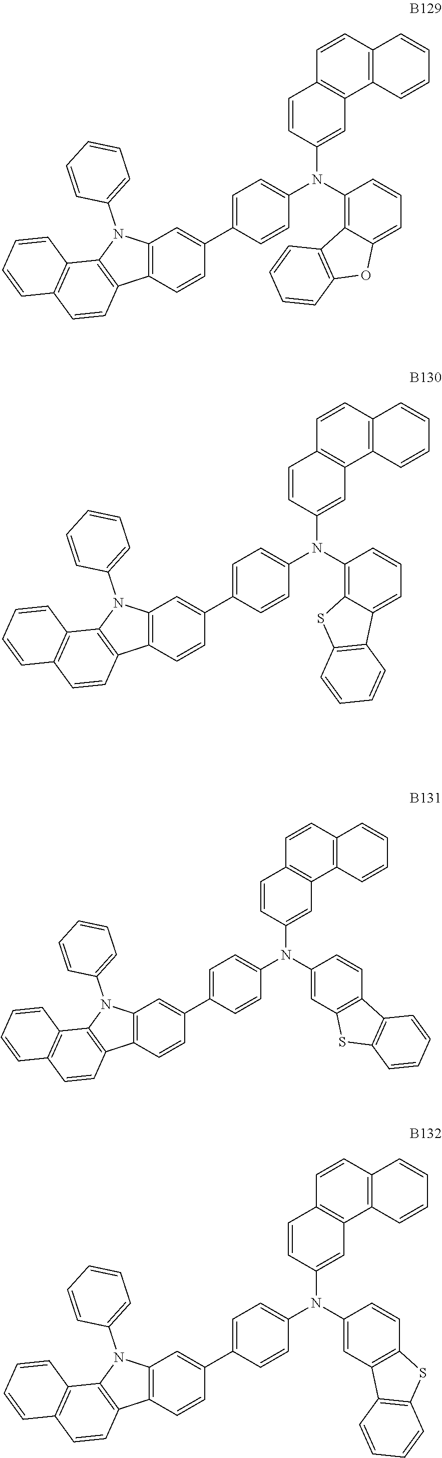 [SMILES: c1ccc(-n2c3cc(-c4ccc(N(c5ccc6ccc7ccccc7c6c5)c5cccc6oc7ccccc7c56)cc4)ccc3c3ccc4ccccc4c32)cc1 ; c1ccc(-n2c3cc(-c4ccc(N(c5ccc6ccc7ccccc7c6c5)c5cccc6c5sc5ccccc56)cc4)ccc3c3ccc4ccccc4c32)cc1 ; c1ccc(-n2c3cc(-c4ccc(N(c5ccc6c(c5)sc5ccccc56)c5ccc6ccc7ccccc7c6c5)cc4)ccc3c3ccc4ccccc4c32)cc1 ; c1ccc(-n2c3cc(-c4ccc(N(c5ccc6ccc7ccccc7c6c5)c5ccc6sc7ccccc7c6c5)cc4)ccc3c3ccc4ccccc4c32)cc1] 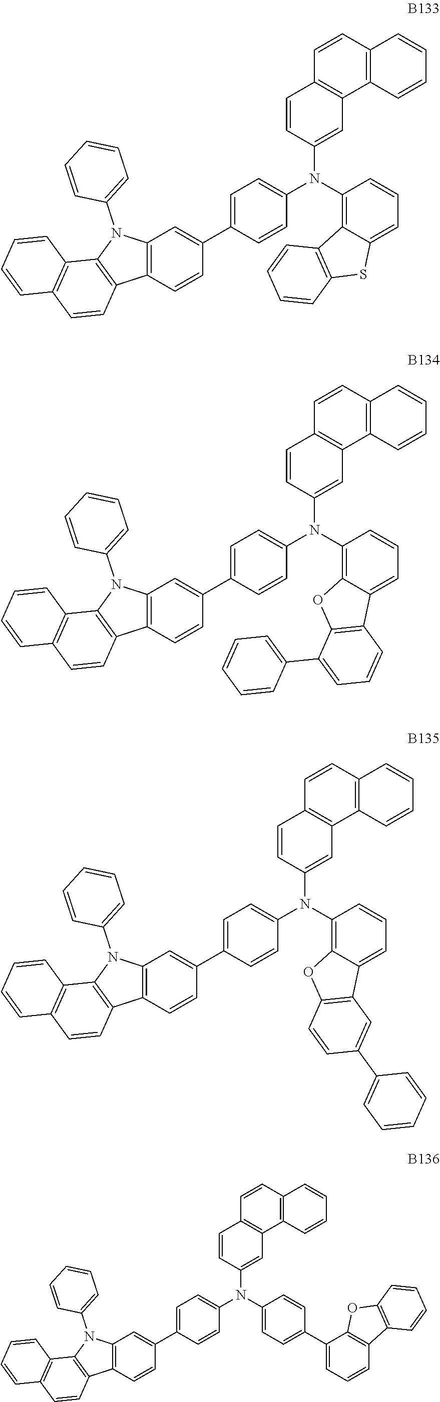 [SMILES: c1ccc(-n2c3cc(-c4ccc(N(c5ccc6ccc7ccccc7c6c5)c5cccc6sc7ccccc7c56)cc4)ccc3c3ccc4ccccc4c32)cc1 ; c1ccc(-c2cccc3c2oc2c(N(c4ccc(-c5ccc6c7ccc8ccccc8c7n(-c7ccccc7)c6c5)cc4)c4ccc5ccc6ccccc6c5c4)cccc23)cc1 ; c1ccc(-c2ccc3oc4c(N(c5ccc(-c6ccc7c8ccc9ccccc9c8n(-c8ccccc8)c7c6)cc5)c5ccc6ccc7ccccc7c6c5)cccc4c3c2)cc1 ; c1ccc(-n2c3cc(-c4ccc(N(c5ccc(-c6cccc7c6oc6ccccc67)cc5)c5ccc6ccc7ccccc7c6c5)cc4)ccc3c3ccc4ccccc4c32)cc1] 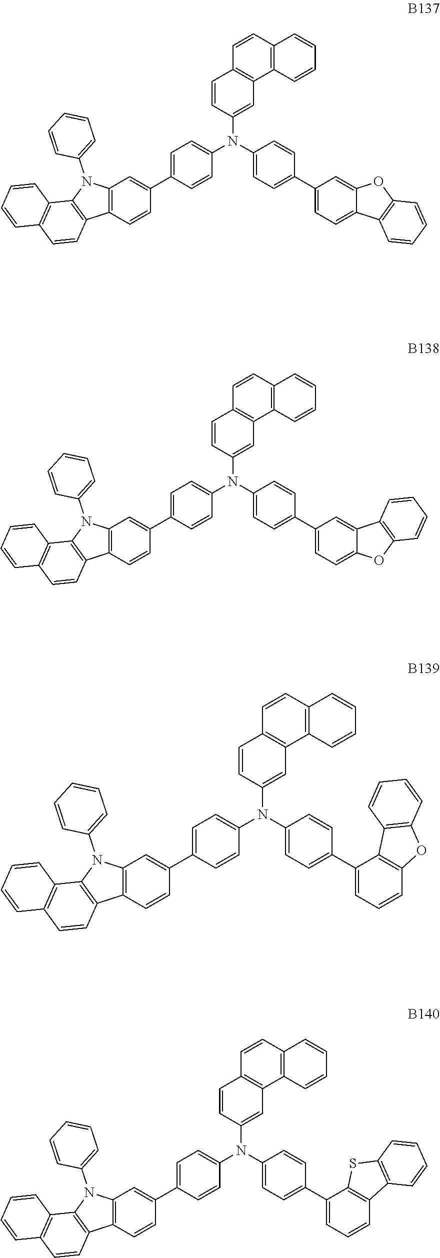 [SMILES: c1ccc(-n2c3cc(-c4ccc(N(c5ccc(-c6ccc7c(c6)oc6ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7oc8ccccc8c7c6)cc5)c5ccc6ccc7ccccc7c6c5)cc4)ccc3c3ccc4ccccc4c32)cc1 ; c1ccc(-n2c3cc(-c4ccc(N(c5ccc(-c6cccc7oc8ccccc8c67)cc5)c5ccc6ccc7ccccc7c6c5)cc4)ccc3c3ccc4ccccc4c32)cc1 ; c1ccc(-n2c3cc(-c4ccc(N(c5ccc(-c6cccc7c6sc6ccccc67)cc5)c5ccc6ccc7ccccc7c6c5)cc4)ccc3c3ccc4ccccc4c32)cc1] 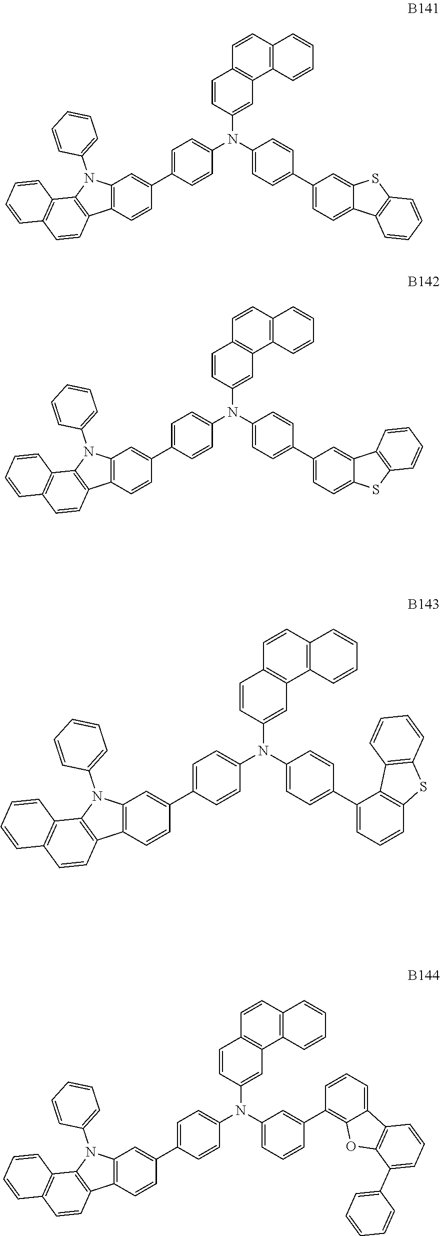 [SMILES: c1ccc(-n2c3cc(-c4ccc(N(c5ccc(-c6ccc7c(c6)sc6ccccc67)cc5)c5ccc6ccc7ccccc7c6c5)cc4)ccc3c3ccc4ccccc4c32)cc1 ; c1ccc(-n2c3cc(-c4ccc(N(c5ccc(-c6ccc7sc8ccccc8c7c6)cc5)c5ccc6ccc7ccccc7c6c5)cc4)ccc3c3ccc4ccccc4c32)cc1 ; c1ccc(-n2c3cc(-c4ccc(N(c5ccc(-c6cccc7sc8ccccc8c67)cc5)c5ccc6ccc7ccccc7c6c5)cc4)ccc3c3ccc4ccccc4c32)cc1 ; c1ccc(-c2cccc3c2oc2c(-c4cccc(N(c5ccc(-c6ccc7c8ccc9ccccc9c8n(-c8ccccc8)c7c6)cc5)c5ccc6ccc7ccccc7c6c5)c4)cccc23)cc1] 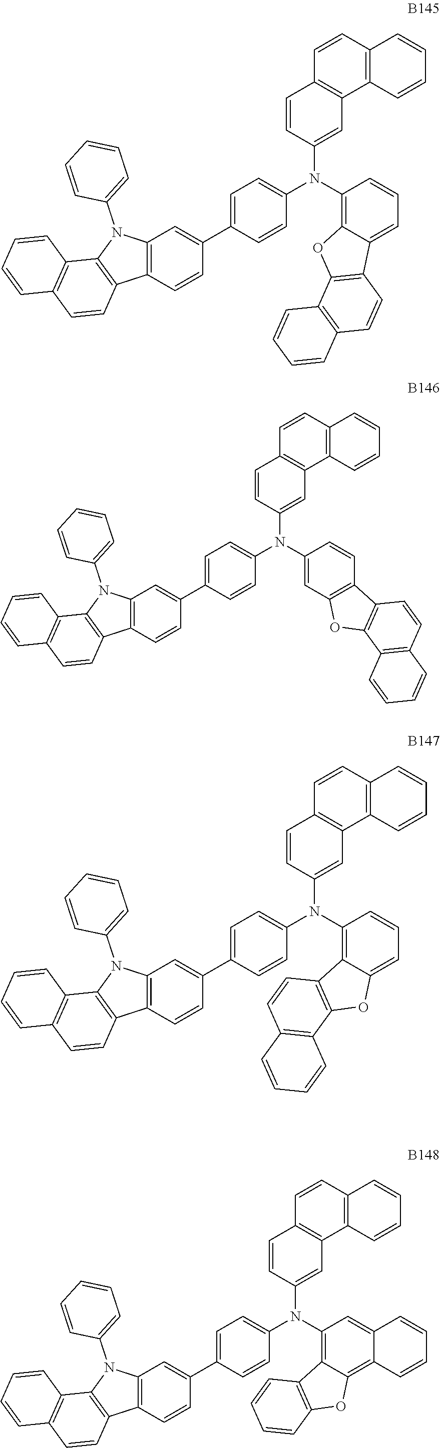 [SMILES: c1ccc(-n2c3cc(-c4ccc(N(c5ccc6ccc7ccccc7c6c5)c5cccc6c5oc5c7ccccc7ccc65)cc4)ccc3c3ccc4ccccc4c32)cc1 ; c1ccc(-n2c3cc(-c4ccc(N(c5ccc6c(c5)oc5c7ccccc7ccc65)c5ccc6ccc7ccccc7c6c5)cc4)ccc3c3ccc4ccccc4c32)cc1 ; c1ccc(-n2c3cc(-c4ccc(N(c5ccc6ccc7ccccc7c6c5)c5cccc6oc7c8ccccc8ccc7c56)cc4)ccc3c3ccc4ccccc4c32)cc1 ; c1ccc(-n2c3cc(-c4ccc(N(c5ccc6ccc7ccccc7c6c5)c5cc6ccccc6c6oc7ccccc7c56)cc4)ccc3c3ccc4ccccc4c32)cc1] 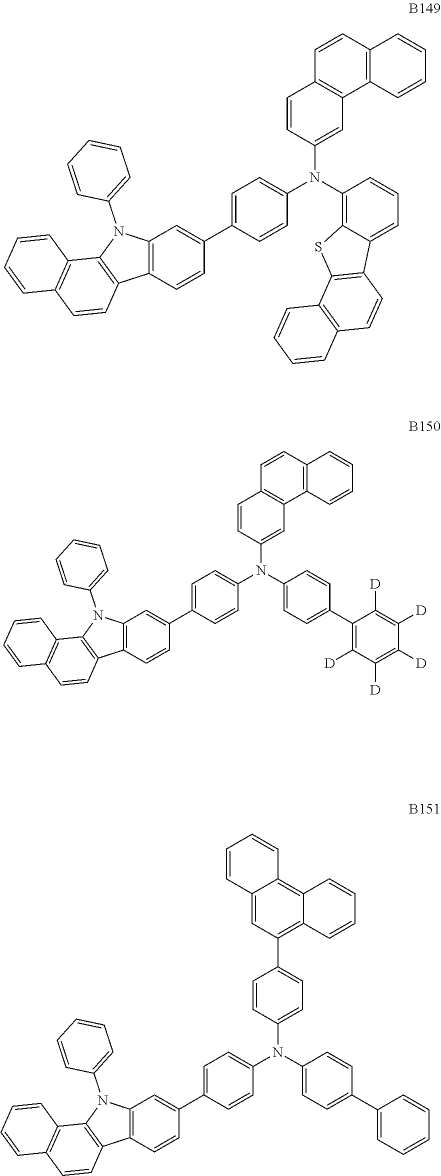 [SMILES: c1ccc(-n2c3cc(-c4ccc(N(c5ccc6ccc7ccccc7c6c5)c5cccc6c5sc5c7ccccc7ccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4ccc5ccccc5c4c3)cc2)c([2H])c1[2H] ; c1ccc(-c2ccc(N(c3ccc(-c4ccc5c6ccc7ccccc7c6n(-c6ccccc6)c5c4)cc3)c3ccc(-c4cc5ccccc5c5ccccc45)cc3)cc2)cc1] 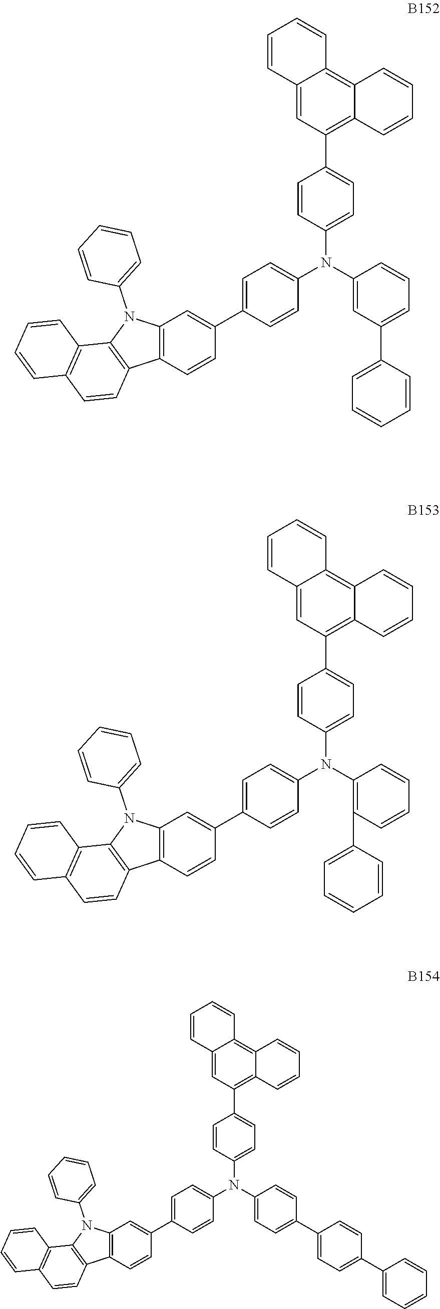 [SMILES: c1ccc(-c2cccc(N(c3ccc(-c4ccc5c6ccc7ccccc7c6n(-c6ccccc6)c5c4)cc3)c3ccc(-c4cc5ccccc5c5ccccc45)cc3)c2)cc1 ; c1ccc(-c2ccccc2N(c2ccc(-c3ccc4c5ccc6ccccc6c5n(-c5ccccc5)c4c3)cc2)c2ccc(-c3cc4ccccc4c4ccccc34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cc6ccccc6c6ccccc56)cc4)cc3)cc2)cc1] 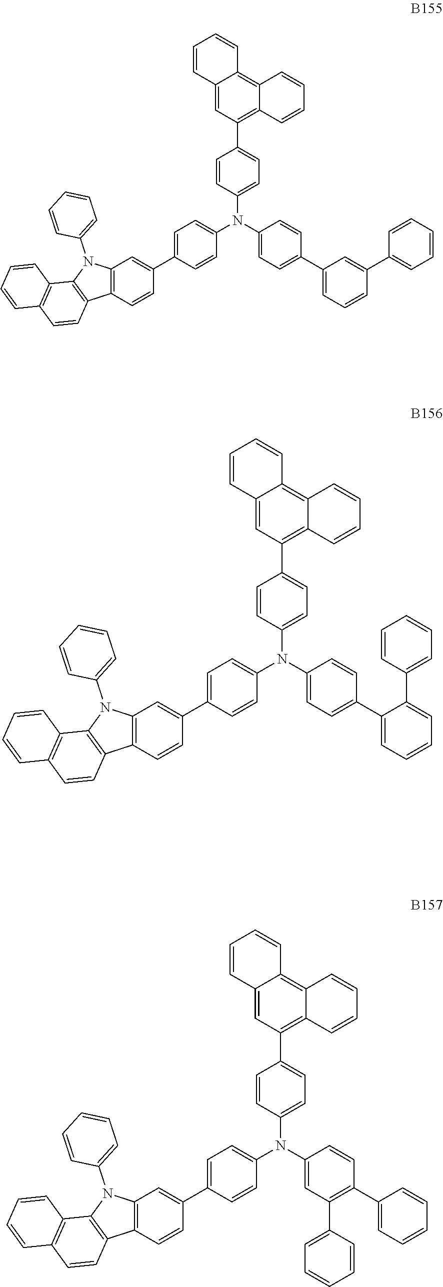 [SMILES: c1ccc(-c2cccc(-c3ccc(N(c4ccc(-c5ccc6c7ccc8ccccc8c7n(-c7ccccc7)c6c5)cc4)c4ccc(-c5cc6ccccc6c6ccccc56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cc5ccccc5c5ccccc45)cc3)cc2)cc1 ; c1ccc(-c2ccc(N(c3ccc(-c4ccc5c6ccc7ccccc7c6n(-c6ccccc6)c5c4)cc3)c3ccc(-c4cc5ccccc5c5ccccc45)cc3)cc2-c2ccccc2)cc1] 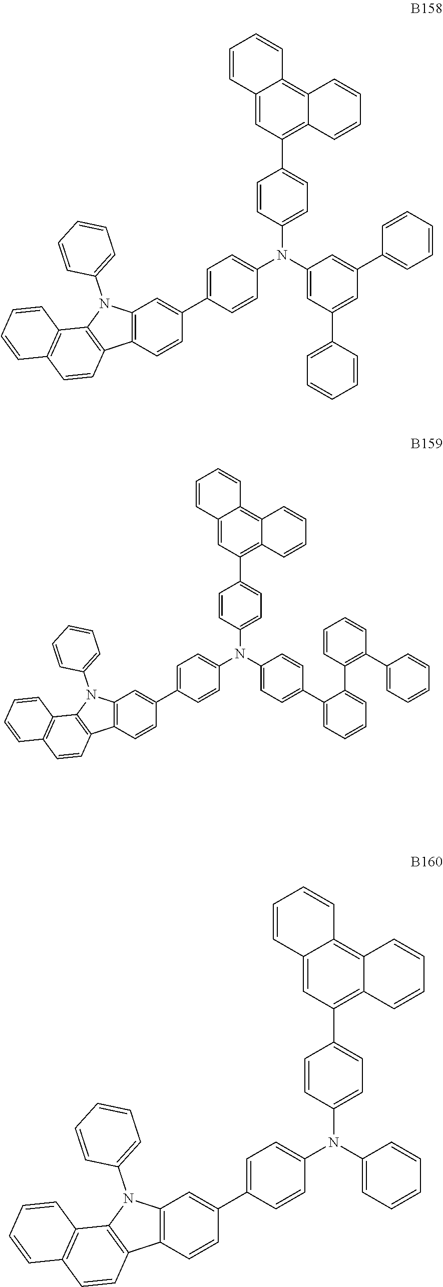 [SMILES: c1ccc(-c2cc(-c3ccccc3)cc(N(c3ccc(-c4ccc5c6ccc7ccccc7c6n(-c6ccccc6)c5c4)cc3)c3ccc(-c4cc5ccccc5c5ccccc45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cc5ccccc5c5ccccc45)cc3)cc2)cc1 ; c1ccc(N(c2ccc(-c3ccc4c5ccc6ccccc6c5n(-c5ccccc5)c4c3)cc2)c2ccc(-c3cc4ccccc4c4ccccc34)cc2)cc1] 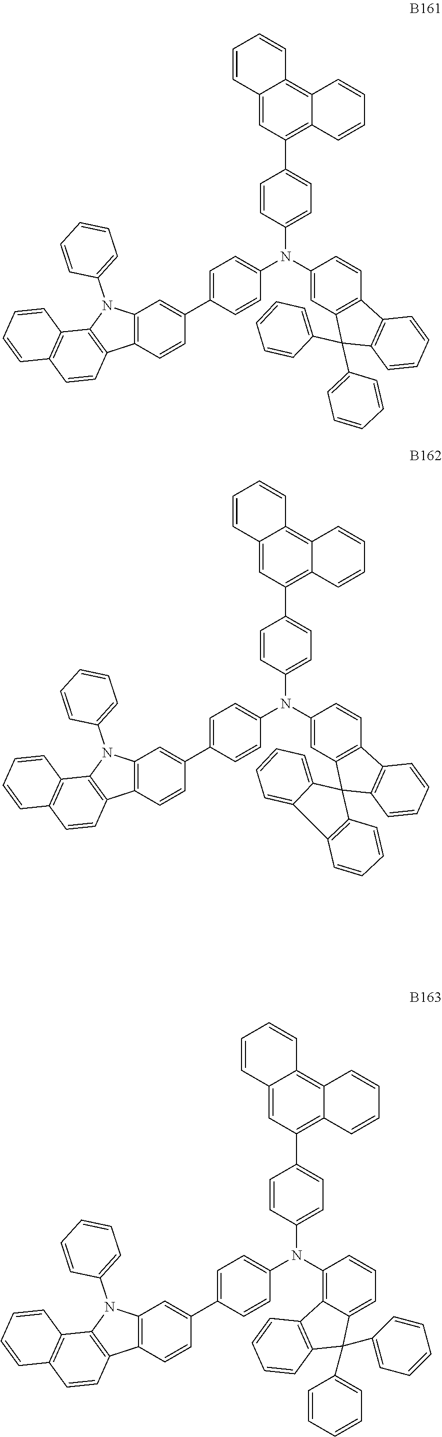 [SMILES: c1ccc(-n2c3cc(-c4ccc(N(c5ccc(-c6cc7ccccc7c7ccccc67)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6cc7ccccc7c7ccccc67)cc5)c5ccc6c(c5)C5(c7ccccc7-c7ccccc75)c5ccccc5-6)cc4)ccc3c3ccc4ccccc4c32)cc1 ; c1ccc(-n2c3cc(-c4ccc(N(c5ccc(-c6cc7ccccc7c7ccccc67)cc5)c5cccc6c5-c5ccccc5C6(c5ccccc5)c5ccccc5)cc4)ccc3c3ccc4ccccc4c32)cc1] 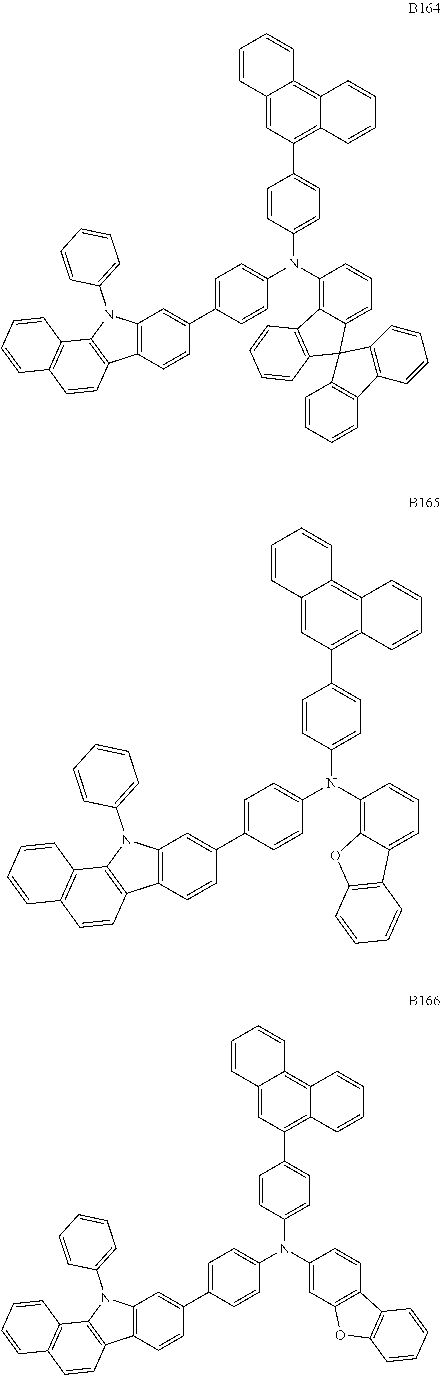 [SMILES: c1ccc(-n2c3cc(-c4ccc(N(c5ccc(-c6cc7ccccc7c7ccccc67)cc5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc(-c6cc7ccccc7c7ccccc67)cc5)c5cccc6c5oc5ccccc56)cc4)ccc3c3ccc4ccccc4c32)cc1 ; c1ccc(-n2c3cc(-c4ccc(N(c5ccc(-c6cc7ccccc7c7ccccc67)cc5)c5ccc6c(c5)oc5ccccc56)cc4)ccc3c3ccc4ccccc4c32)cc1] 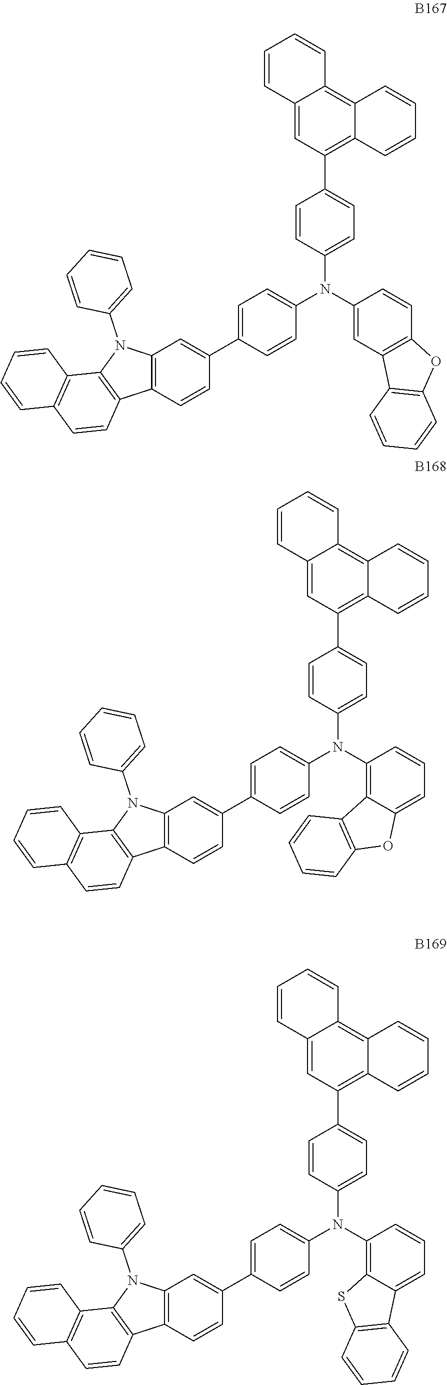 [SMILES: c1ccc(-n2c3cc(-c4ccc(N(c5ccc(-c6cc7ccccc7c7ccccc67)cc5)c5ccc6oc7ccccc7c6c5)cc4)ccc3c3ccc4ccccc4c32)cc1 ; c1ccc(-n2c3cc(-c4ccc(N(c5ccc(-c6cc7ccccc7c7ccccc67)cc5)c5cccc6oc7ccccc7c56)cc4)ccc3c3ccc4ccccc4c32)cc1 ; c1ccc(-n2c3cc(-c4ccc(N(c5ccc(-c6cc7ccccc7c7ccccc67)cc5)c5cccc6c5sc5ccccc56)cc4)ccc3c3ccc4ccccc4c32)cc1] 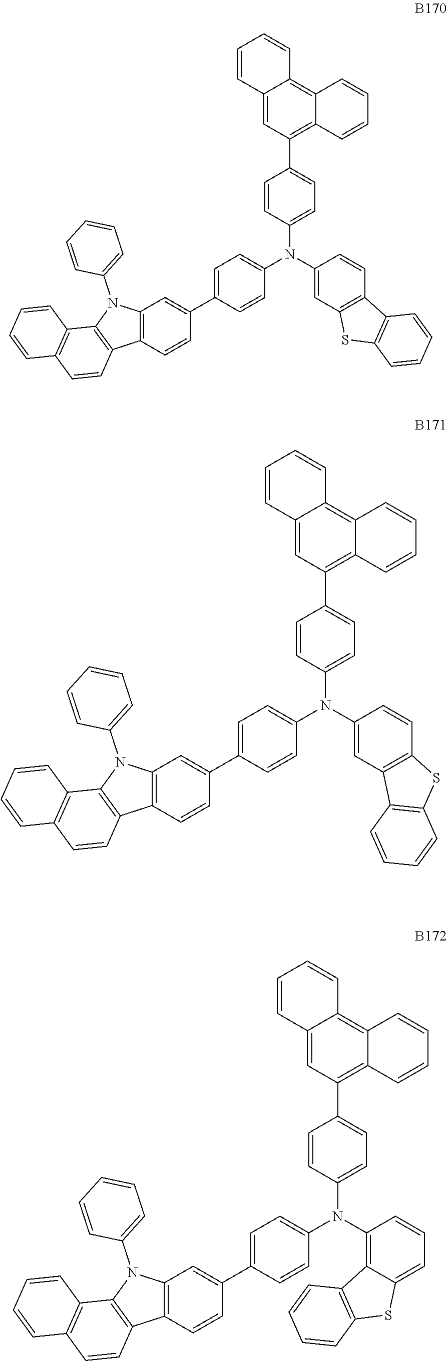 [SMILES: c1ccc(-n2c3cc(-c4ccc(N(c5ccc(-c6cc7ccccc7c7ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6cc7ccccc7c7ccccc67)cc5)c5ccc6sc7ccccc7c6c5)cc4)ccc3c3ccc4ccccc4c32)cc1 ; c1ccc(-n2c3cc(-c4ccc(N(c5ccc(-c6cc7ccccc7c7ccccc67)cc5)c5cccc6sc7ccccc7c56)cc4)ccc3c3ccc4ccccc4c32)cc1] 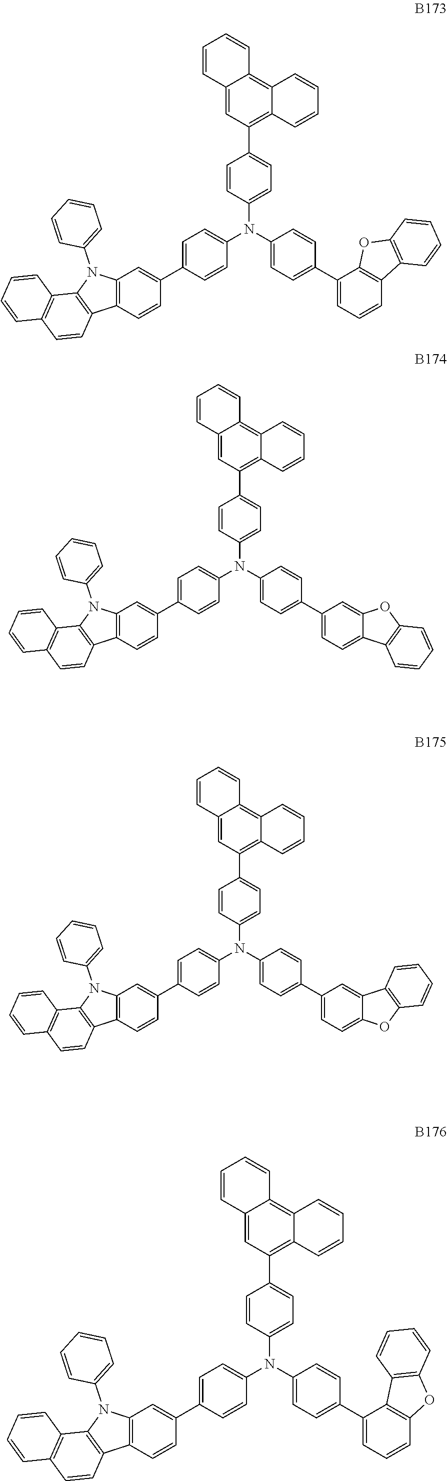 [SMILES: c1ccc(-n2c3cc(-c4ccc(N(c5ccc(-c6cc7ccccc7c7ccccc67)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7c(c6)oc6ccccc67)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6ccc7oc8ccccc8c7c6)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6cc7ccccc7c7ccccc67)cc5)c5ccc(-c6cccc7oc8ccccc8c67)cc5)cc4)ccc3c3ccc4ccccc4c32)cc1] 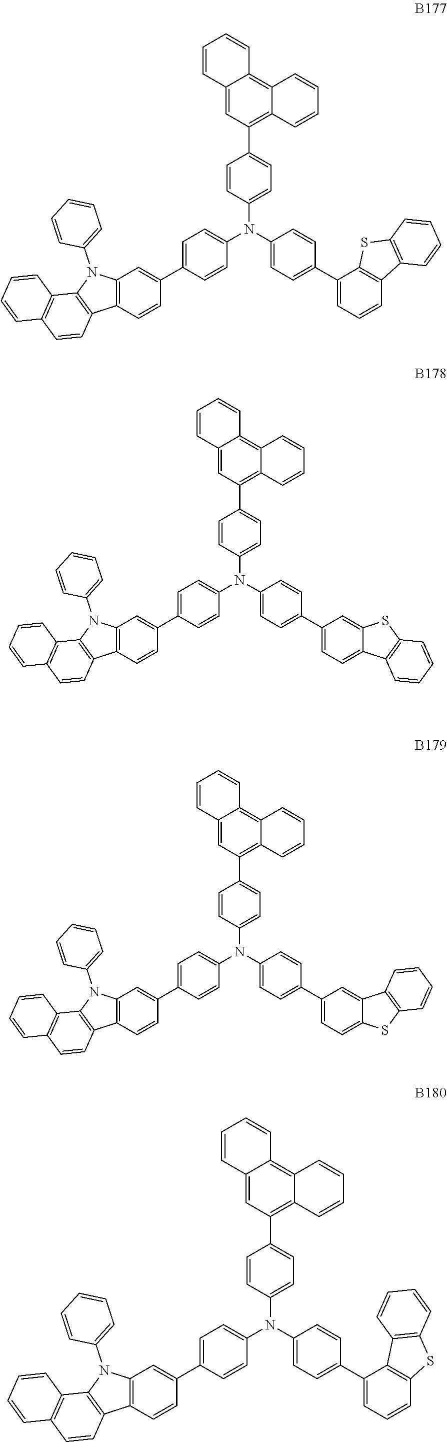 [SMILES: c1ccc(-n2c3cc(-c4ccc(N(c5ccc(-c6cc7ccccc7c7ccccc67)cc5)c5ccc(-c6cccc7c6sc6ccccc67)cc5)cc4)ccc3c3ccc4ccccc4c32)cc1 ; c1ccc(-n2c3cc(-c4ccc(N(c5ccc(-c6ccc7c(c6)sc6ccccc67)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6ccc7sc8ccccc8c7c6)cc5)c5ccc(-c6cc7ccccc7c7ccccc67)cc5)cc4)ccc3c3ccc4ccccc4c32)cc1 ; c1ccc(-n2c3cc(-c4ccc(N(c5ccc(-c6cc7ccccc7c7ccccc67)cc5)c5ccc(-c6cccc7sc8ccccc8c67)cc5)cc4)ccc3c3ccc4ccccc4c32)cc1] 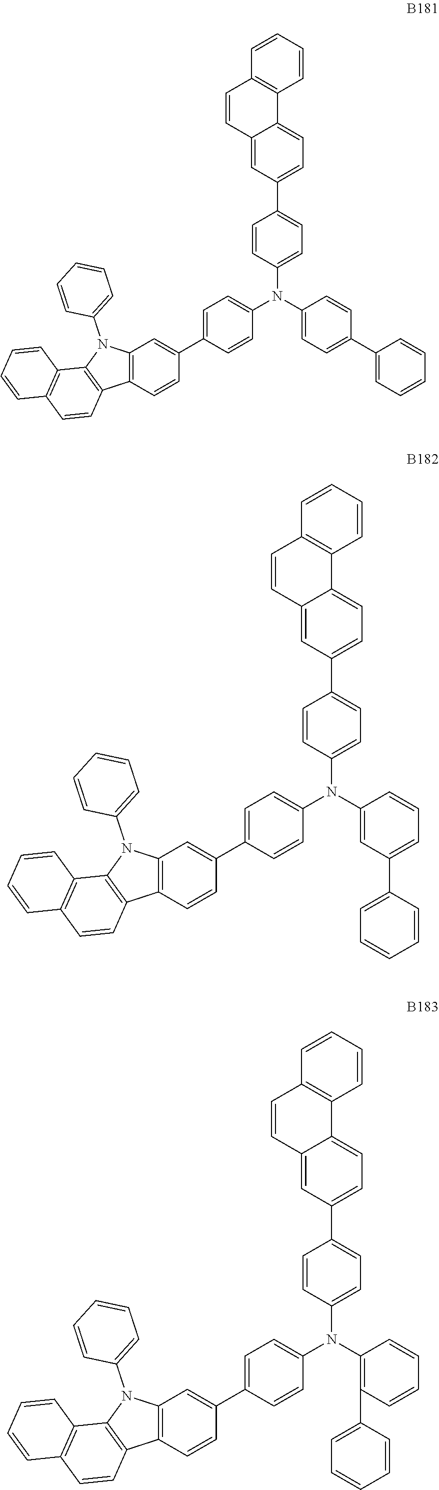 [SMILES: c1ccc(-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5c(ccc6ccccc65)c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4c(ccc5ccccc54)c3)cc2)c2ccc(-c3ccc4c5ccc6ccccc6c5n(-c5ccccc5)c4c3)cc2)cc1] 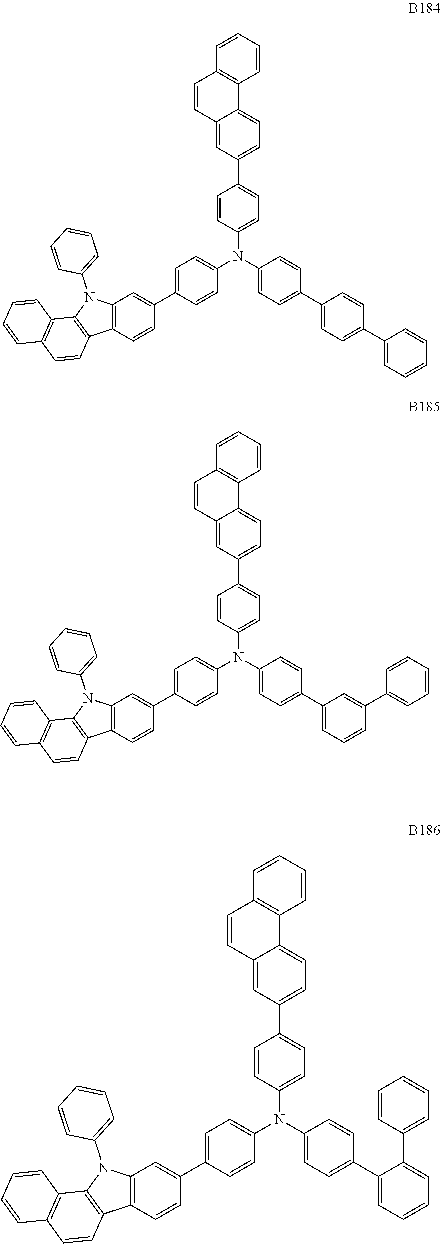 [SMILES: c1ccc(-c2ccc(-c3ccc(N(c4ccc(-c5ccc6c(ccc7ccccc76)c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6c(ccc7ccccc76)c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2)cc1] 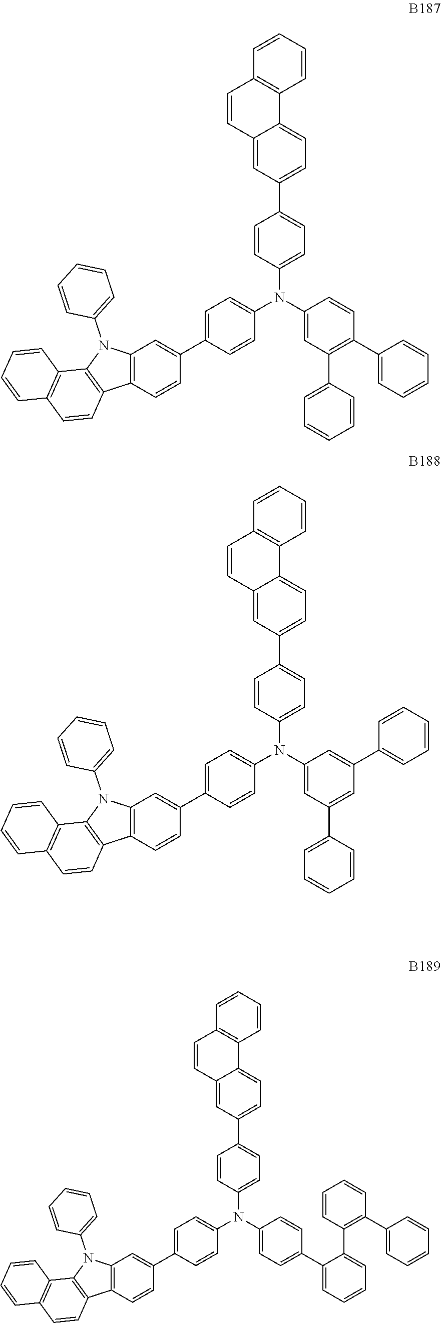 [SMILES: c1ccc(-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5c(ccc6ccccc65)c4)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5c(ccc6ccccc65)c4)cc3)c3ccc(-c4ccc5c6ccc7ccccc7c6n(-c6ccccc6)c5c4)cc3)cc2)cc1] 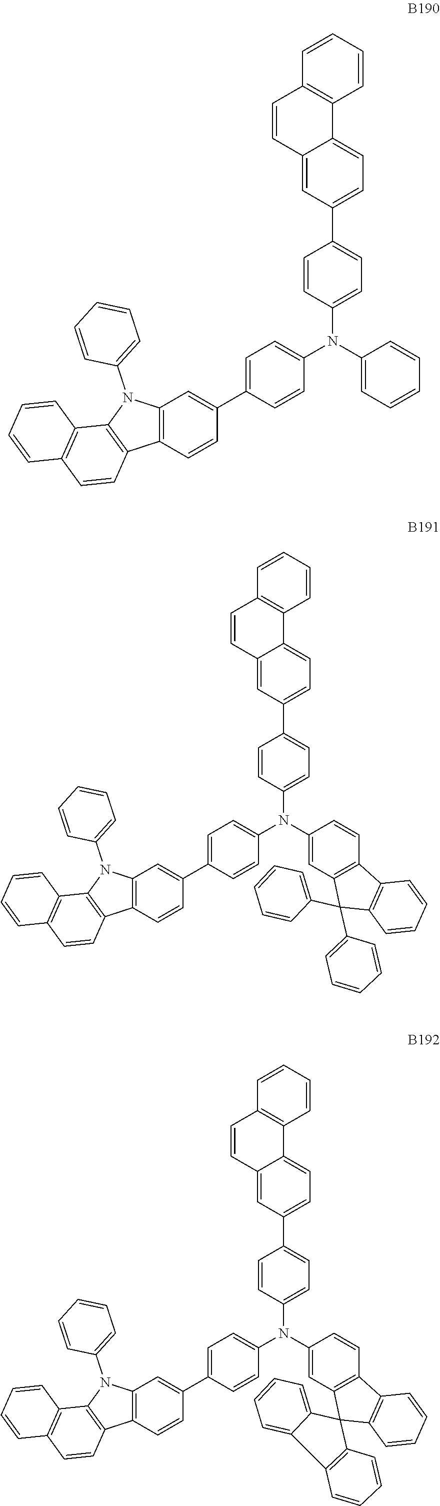 [SMILES: c1ccc(N(c2ccc(-c3ccc4c(ccc5ccccc54)c3)cc2)c2ccc(-c3ccc4c5ccc6ccccc6c5n(-c5ccccc5)c4c3)cc2)cc1 ; c1ccc(-n2c3cc(-c4ccc(N(c5ccc(-c6ccc7c(ccc8ccccc87)c6)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6c(c5)C5(c7ccccc7-c7ccccc75)c5ccccc5-6)cc4)ccc3c3ccc4ccccc4c32)cc1] 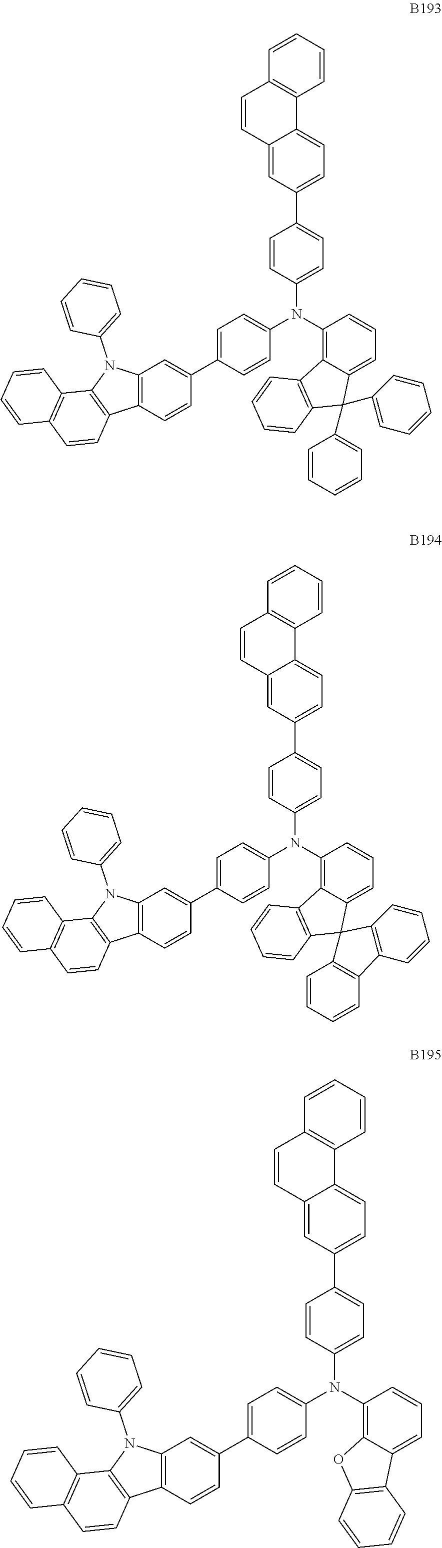 [SMILES: c1ccc(-n2c3cc(-c4ccc(N(c5ccc(-c6ccc7c(ccc8ccccc87)c6)cc5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc(-c6ccc7c(ccc8ccccc87)c6)cc5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc(-c6ccc7c(ccc8ccccc87)c6)cc5)c5cccc6c5oc5ccccc56)cc4)ccc3c3ccc4ccccc4c32)cc1] 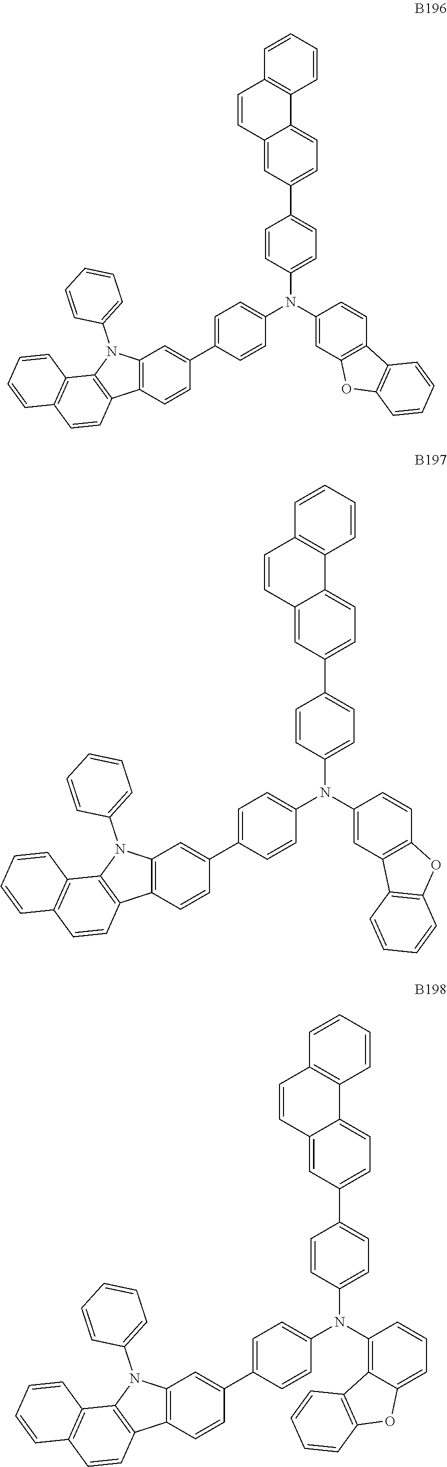 [SMILES: c1ccc(-n2c3cc(-c4ccc(N(c5ccc(-c6ccc7c(ccc8ccccc87)c6)cc5)c5ccc6c(c5)oc5ccccc56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6oc7ccccc7c6c5)cc4)ccc3c3ccc4ccccc4c32)cc1 ; c1ccc(-n2c3cc(-c4ccc(N(c5ccc(-c6ccc7c(ccc8ccccc87)c6)cc5)c5cccc6oc7ccccc7c56)cc4)ccc3c3ccc4ccccc4c32)cc1] 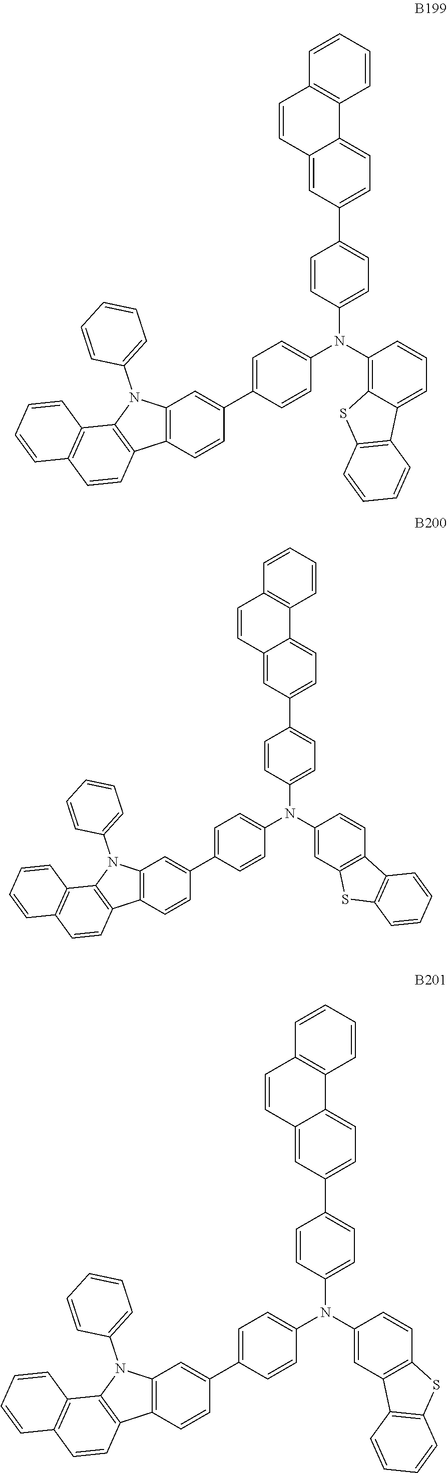 [SMILES: c1ccc(-n2c3cc(-c4ccc(N(c5ccc(-c6ccc7c(ccc8ccccc87)c6)cc5)c5cccc6c5sc5ccccc56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6c(c5)sc5ccccc56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc6sc7ccccc7c6c5)cc4)ccc3c3ccc4ccccc4c32)cc1] 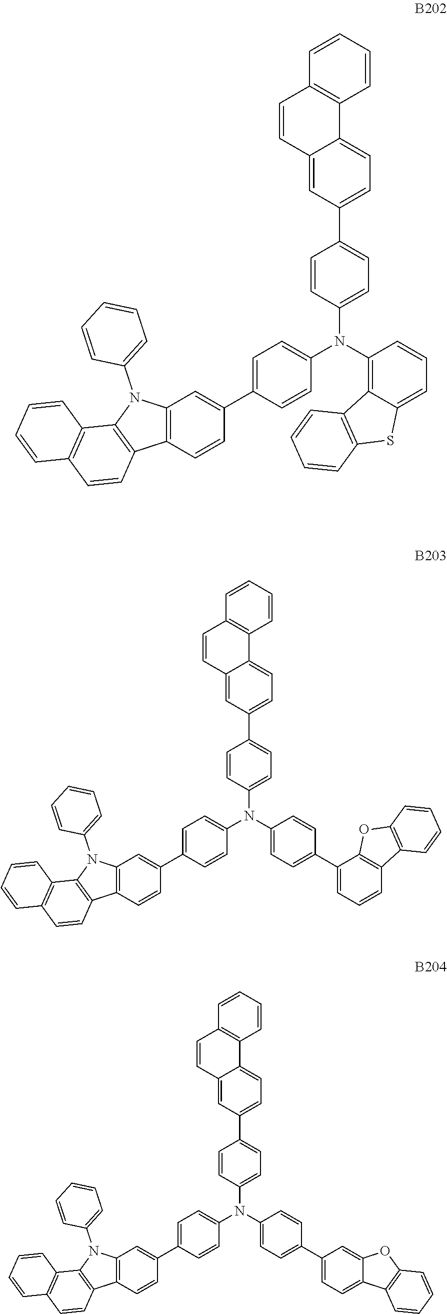 [SMILES: c1ccc(-n2c3cc(-c4ccc(N(c5ccc(-c6ccc7c(ccc8ccccc87)c6)cc5)c5cccc6sc7ccccc7c56)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6ccc7c(c6)oc6ccccc67)cc5)cc4)ccc3c3ccc4ccccc4c32)cc1] 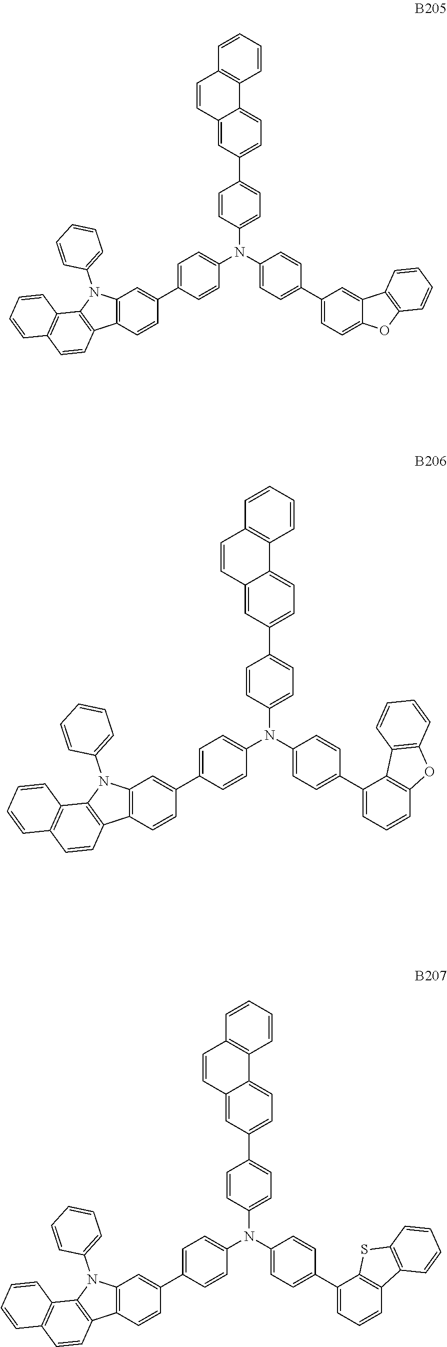 [SMILES: c1ccc(-n2c3cc(-c4ccc(N(c5ccc(-c6ccc7c(ccc8ccccc87)c6)cc5)c5ccc(-c6ccc7oc8ccccc8c7c6)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7oc8ccccc8c67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7c6sc6ccccc67)cc5)cc4)ccc3c3ccc4ccccc4c32)cc1] 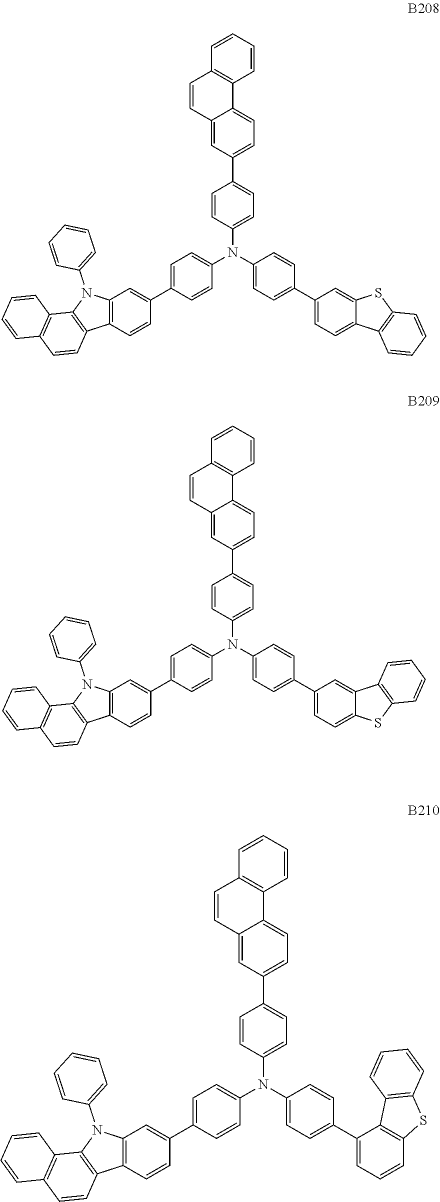 [SMILES: c1ccc(-n2c3cc(-c4ccc(N(c5ccc(-c6ccc7c(ccc8ccccc87)c6)cc5)c5ccc(-c6ccc7c(c6)sc6ccccc67)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6ccc7sc8ccccc8c7c6)cc5)cc4)ccc3c3ccc4ccccc4c32)cc1 ; c1ccc(-n2c3cc(-c4ccc(N(c5ccc(-c6ccc7c(ccc8ccccc87)c6)cc5)c5ccc(-c6cccc7sc8ccccc8c67)cc5)cc4)ccc3c3ccc4ccccc4c32)cc1] 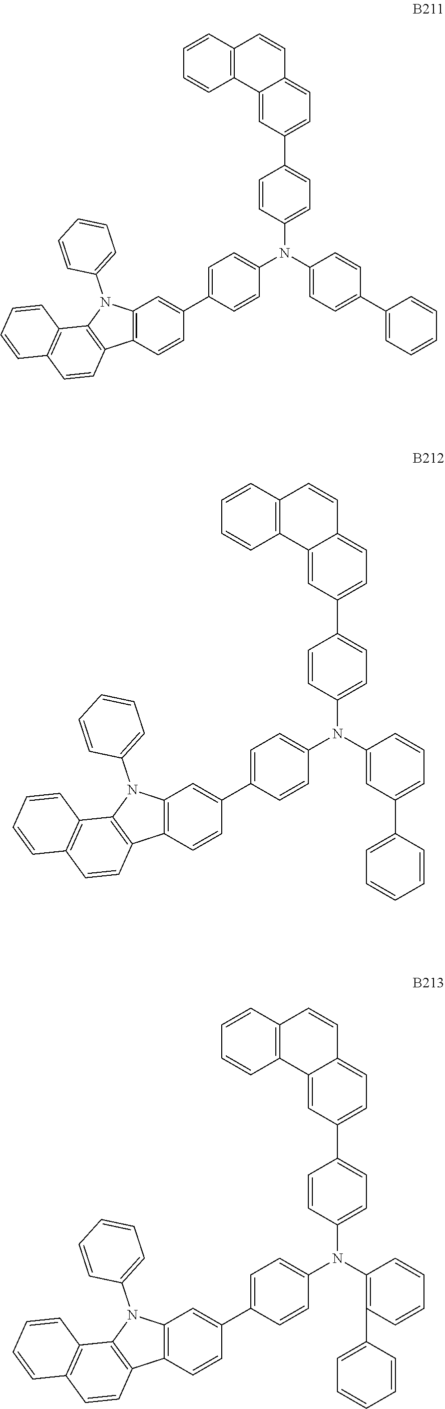 [SMILES: c1ccc(-c2ccc(N(c3ccc(-c4ccc5ccc6ccccc6c5c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5ccc6ccccc6c5c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4ccc5ccccc5c4c3)cc2)c2ccc(-c3ccc4c5ccc6ccccc6c5n(-c5ccccc5)c4c3)cc2)cc1] 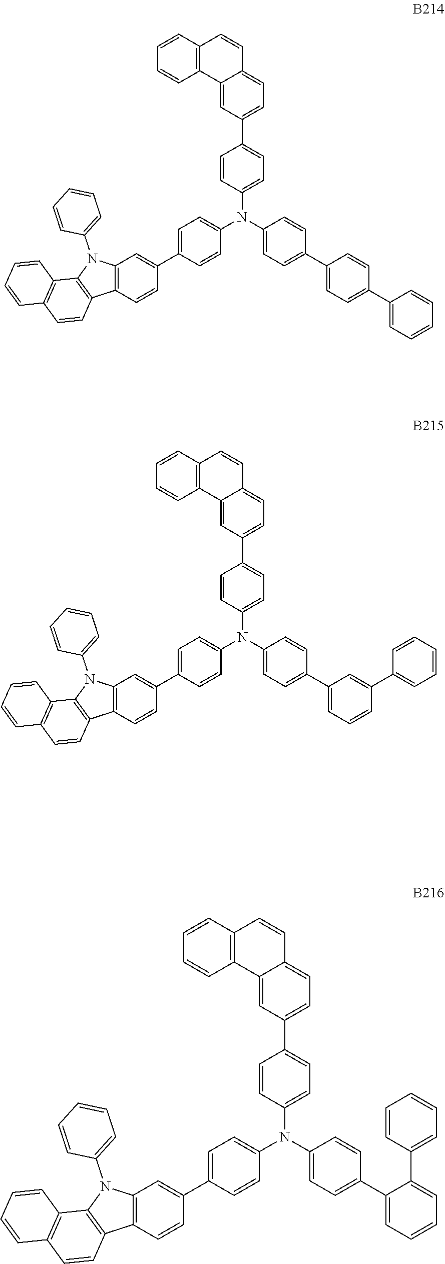 [SMILES: c1ccc(-c2ccc(-c3ccc(N(c4ccc(-c5ccc6ccc7ccccc7c6c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6ccc7ccccc7c6c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5ccc6ccccc6c5c4)cc3)c3ccc(-c4ccc5c6ccc7ccccc7c6n(-c6ccccc6)c5c4)cc3)cc2)cc1] 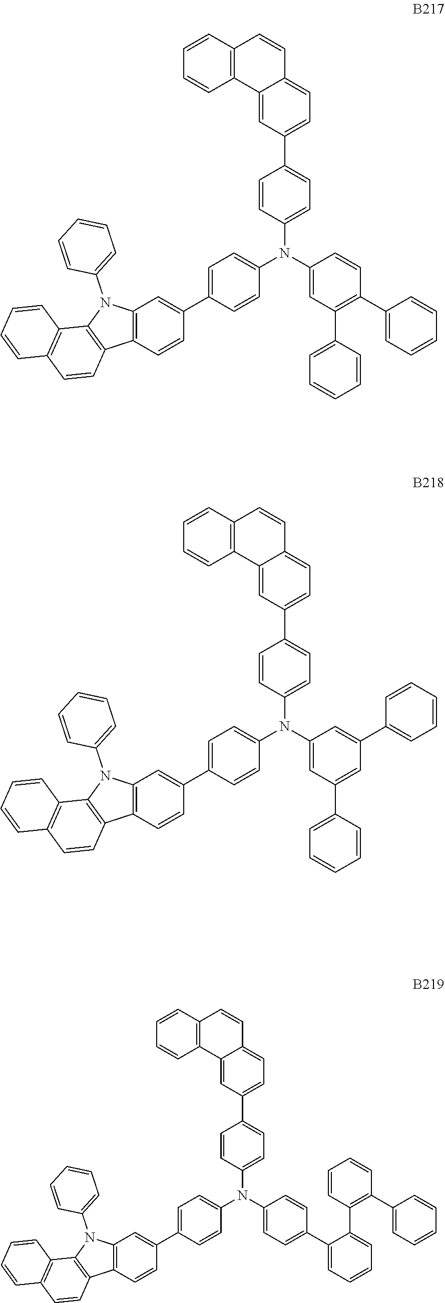 [SMILES: c1ccc(-c2ccc(N(c3ccc(-c4ccc5ccc6ccccc6c5c4)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5ccc6ccccc6c5c4)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5ccc6ccccc6c5c4)cc3)c3ccc(-c4ccc5c6ccc7ccccc7c6n(-c6ccccc6)c5c4)cc3)cc2)cc1] 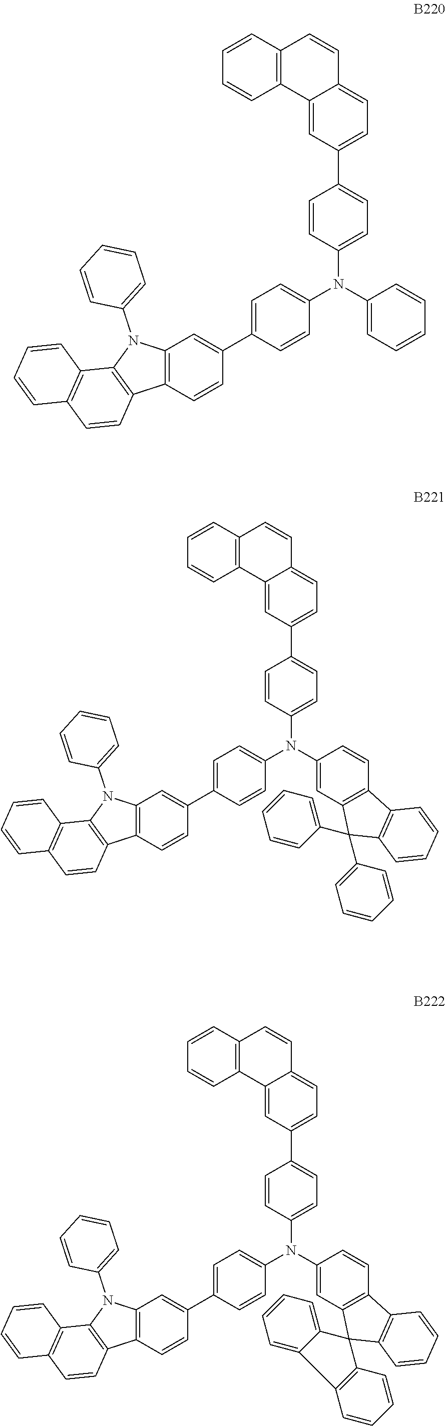 [SMILES: c1ccc(N(c2ccc(-c3ccc4ccc5ccccc5c4c3)cc2)c2ccc(-c3ccc4c5ccc6ccccc6c5n(-c5ccccc5)c4c3)cc2)cc1 ; c1ccc(-n2c3cc(-c4ccc(N(c5ccc(-c6ccc7ccc8ccccc8c7c6)cc5)c5ccc6c(c5)C(c5ccccc5)(c5ccccc5)c5ccccc5-6)cc4)ccc3c3ccc4ccccc4c32)cc1 ; c1ccc(-n2c3cc(-c4ccc(N(c5ccc(-c6ccc7ccc8ccccc8c7c6)cc5)c5ccc6c(c5)C5(c7ccccc7-c7ccccc75)c5ccccc5-6)cc4)ccc3c3ccc4ccccc4c32)cc1] 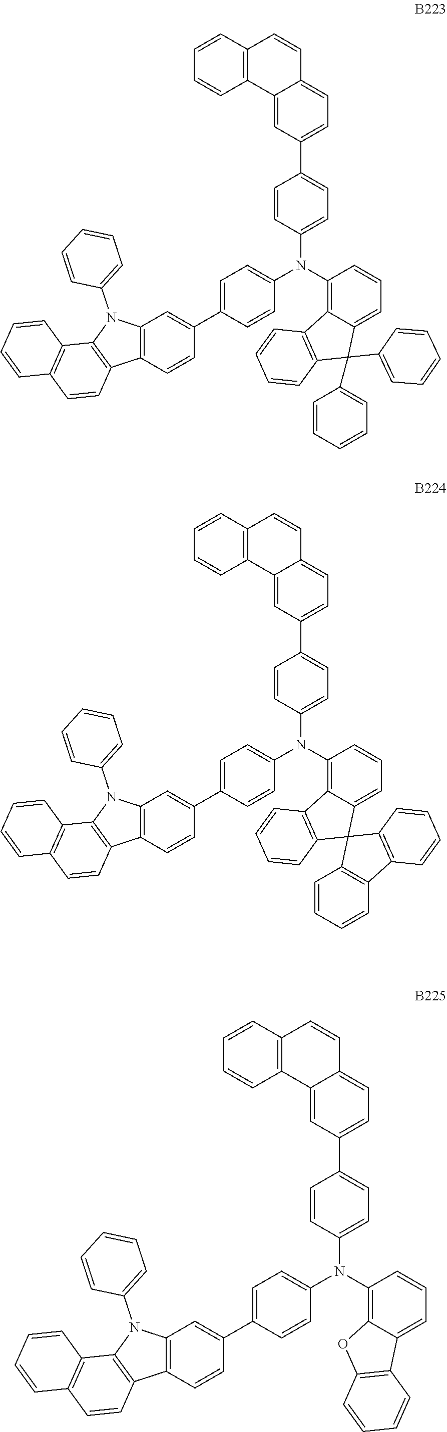 [SMILES: c1ccc(-n2c3cc(-c4ccc(N(c5ccc(-c6ccc7ccc8ccccc8c7c6)cc5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc(-c6ccc7ccc8ccccc8c7c6)cc5)c5cccc6c5-c5ccccc5C65c6ccccc6-c6ccccc65)cc4)ccc3c3ccc4ccccc4c32)cc1 ; c1ccc(-n2c3cc(-c4ccc(N(c5ccc(-c6ccc7ccc8ccccc8c7c6)cc5)c5cccc6c5oc5ccccc56)cc4)ccc3c3ccc4ccccc4c32)cc1] 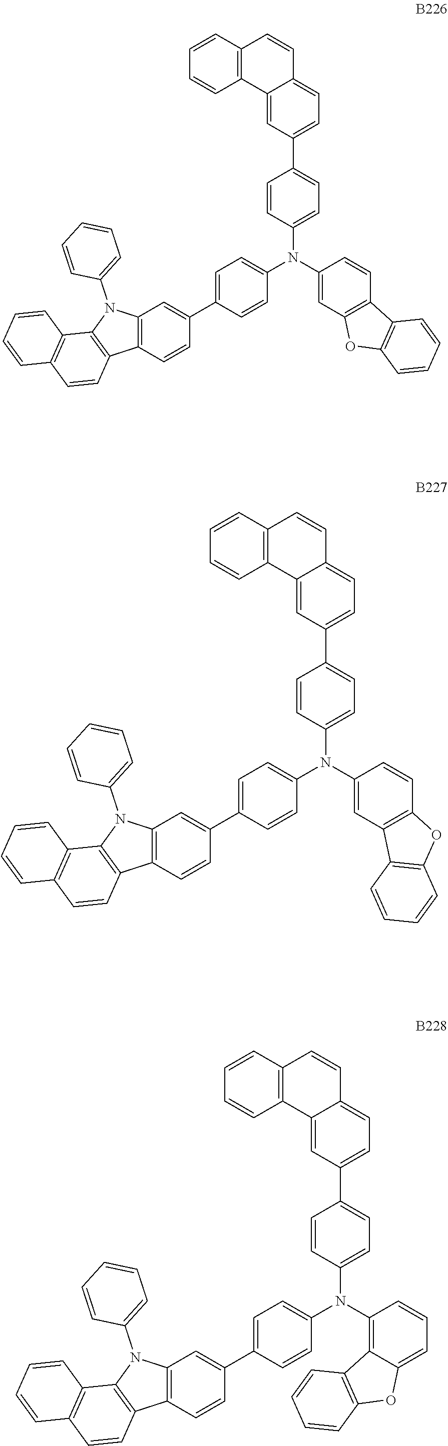 [SMILES: c1ccc(-n2c3cc(-c4ccc(N(c5ccc(-c6ccc7ccc8ccccc8c7c6)cc5)c5ccc6c(c5)oc5ccccc56)cc4)ccc3c3ccc4ccccc4c32)cc1 ; c1ccc(-n2c3cc(-c4ccc(N(c5ccc(-c6ccc7ccc8ccccc8c7c6)cc5)c5ccc6oc7ccccc7c6c5)cc4)ccc3c3ccc4ccccc4c32)cc1 ; c1ccc(-n2c3cc(-c4ccc(N(c5ccc(-c6ccc7ccc8ccccc8c7c6)cc5)c5cccc6oc7ccccc7c56)cc4)ccc3c3ccc4ccccc4c32)cc1] 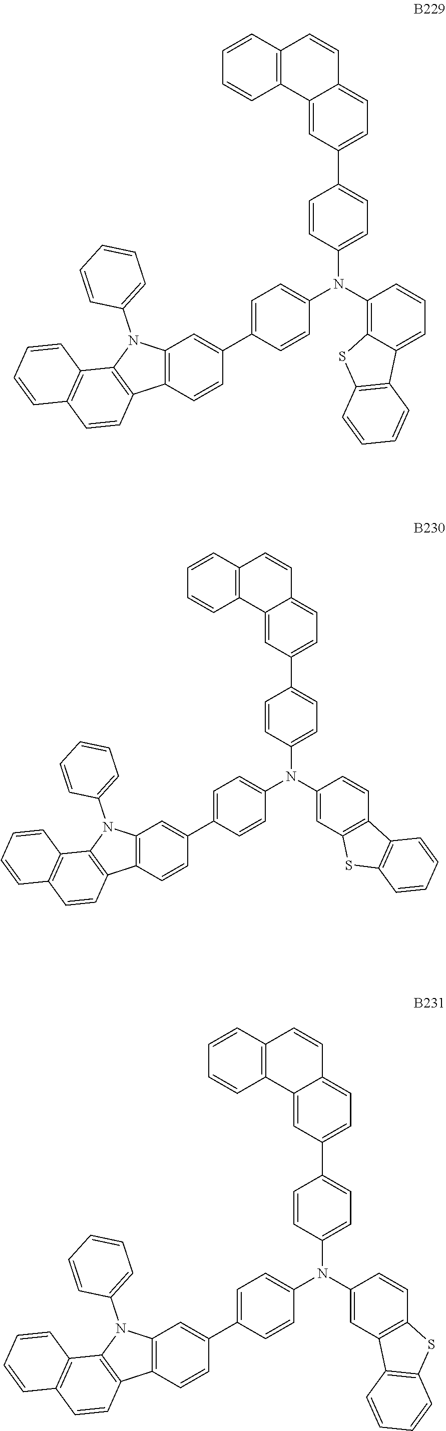 [SMILES: c1ccc(-n2c3cc(-c4ccc(N(c5ccc(-c6ccc7ccc8ccccc8c7c6)cc5)c5cccc6c5sc5ccccc56)cc4)ccc3c3ccc4ccccc4c32)cc1 ; c1ccc(-n2c3cc(-c4ccc(N(c5ccc(-c6ccc7ccc8ccccc8c7c6)cc5)c5ccc6c(c5)sc5ccccc56)cc4)ccc3c3ccc4ccccc4c32)cc1 ; c1ccc(-n2c3cc(-c4ccc(N(c5ccc(-c6ccc7ccc8ccccc8c7c6)cc5)c5ccc6sc7ccccc7c6c5)cc4)ccc3c3ccc4ccccc4c32)cc1] 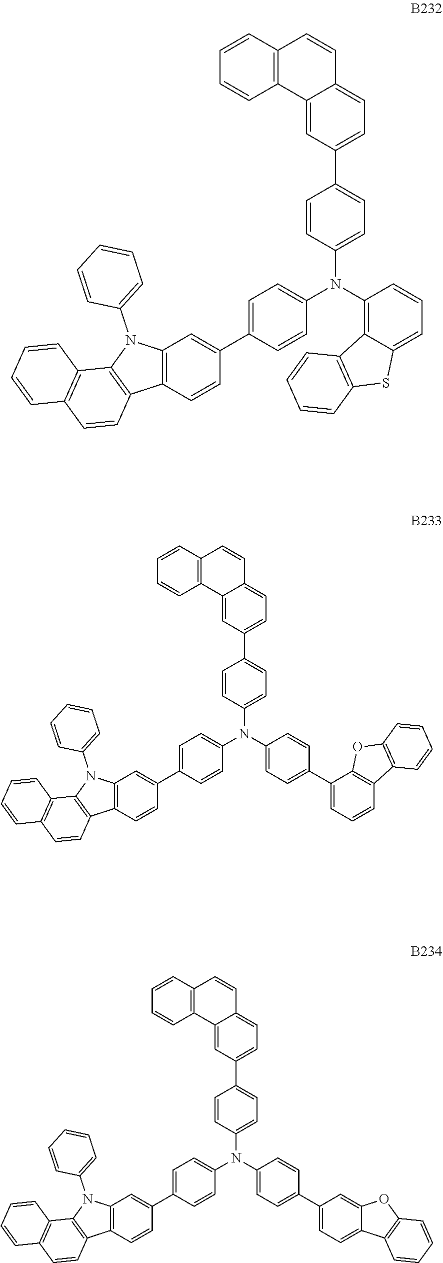 [SMILES: c1ccc(-n2c3cc(-c4ccc(N(c5ccc(-c6ccc7ccc8ccccc8c7c6)cc5)c5cccc6sc7ccccc7c56)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6cccc7c6oc6ccccc67)cc5)cc4)ccc3c3ccc4ccccc4c32)cc1 ; c1ccc(-n2c3cc(-c4ccc(N(c5ccc(-c6ccc7c(c6)oc6ccccc67)cc5)c5ccc(-c6ccc7ccc8ccccc8c7c6)cc5)cc4)ccc3c3ccc4ccccc4c32)cc1] 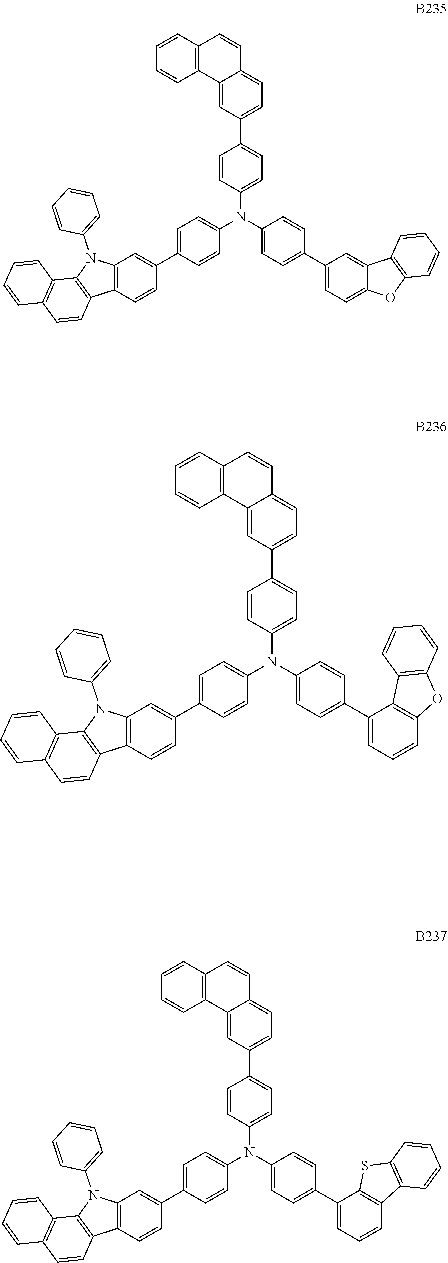 [SMILES: c1ccc(-n2c3cc(-c4ccc(N(c5ccc(-c6ccc7ccc8ccccc8c7c6)cc5)c5ccc(-c6ccc7oc8ccccc8c7c6)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6cccc7oc8ccccc8c67)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6cccc7c6sc6ccccc67)cc5)cc4)ccc3c3ccc4ccccc4c32)cc1] 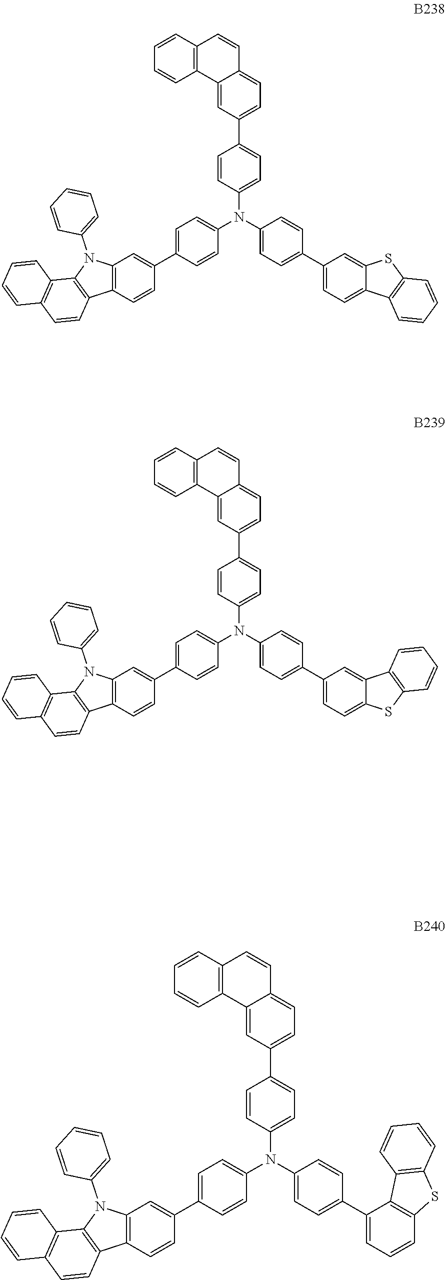 [SMILES: c1ccc(-n2c3cc(-c4ccc(N(c5ccc(-c6ccc7c(c6)sc6ccccc67)cc5)c5ccc(-c6ccc7ccc8ccccc8c7c6)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6ccc7sc8ccccc8c7c6)cc5)cc4)ccc3c3ccc4ccccc4c32)cc1 ; c1ccc(-n2c3cc(-c4ccc(N(c5ccc(-c6ccc7ccc8ccccc8c7c6)cc5)c5ccc(-c6cccc7sc8ccccc8c67)cc5)cc4)ccc3c3ccc4ccccc4c32)cc1] 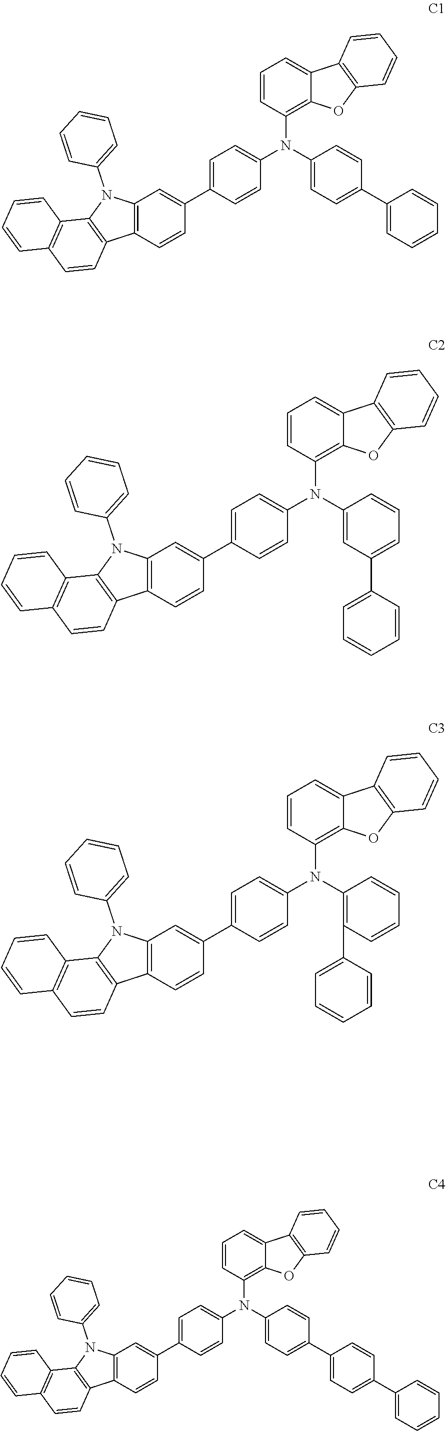 [SMILES: c1ccc(-c2ccc(N(c3ccc(-c4ccc5c6ccc7ccccc7c6n(-c6ccccc6)c5c4)cc3)c3cccc4c3oc3ccccc34)cc2)cc1 ; c1ccc(-c2cccc(N(c3ccc(-c4ccc5c6ccc7ccccc7c6n(-c6ccccc6)c5c4)cc3)c3cccc4c3oc3ccccc34)c2)cc1 ; c1ccc(-c2ccccc2N(c2ccc(-c3ccc4c5ccc6ccccc6c5n(-c5ccccc5)c4c3)cc2)c2cccc3c2oc2ccccc23)cc1 ; c1ccc(-c2ccc(-c3ccc(N(c4ccc(-c5ccc6c7ccc8ccccc8c7n(-c7ccccc7)c6c5)cc4)c4cccc5c4oc4ccccc45)cc3)cc2)cc1] 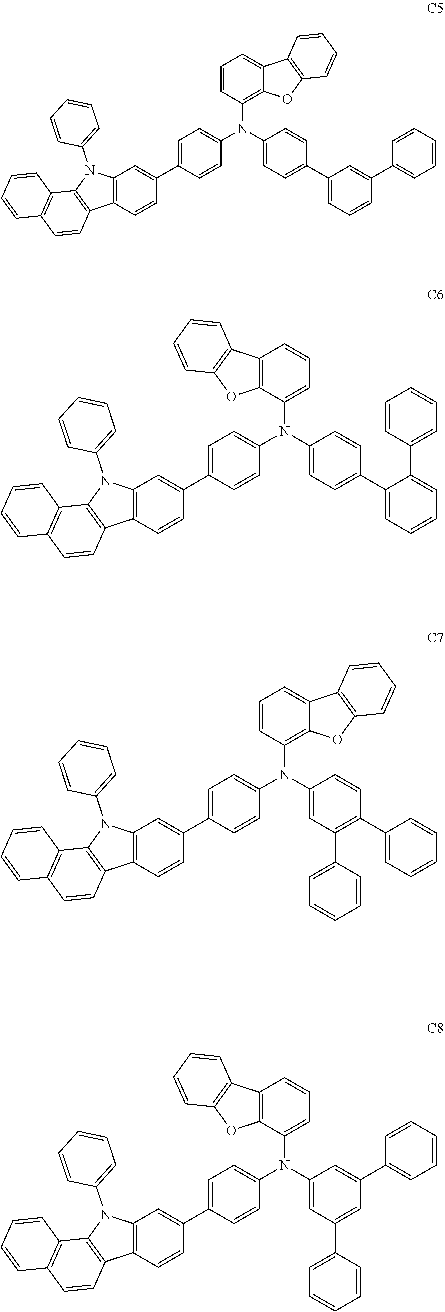 [SMILES: c1ccc(-c2cccc(-c3ccc(N(c4ccc(-c5ccc6c7ccc8ccccc8c7n(-c7ccccc7)c6c5)cc4)c4cccc5c4oc4ccccc45)cc3)c2)cc1 ; c1ccc(-c2ccccc2-c2ccc(N(c3ccc(-c4ccc5c6ccc7ccccc7c6n(-c6ccccc6)c5c4)cc3)c3cccc4c3oc3ccccc34)cc2)cc1 ; c1ccc(-c2ccc(N(c3ccc(-c4ccc5c6ccc7ccccc7c6n(-c6ccccc6)c5c4)cc3)c3cccc4c3oc3ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4c3oc3ccccc34)c2)cc1] 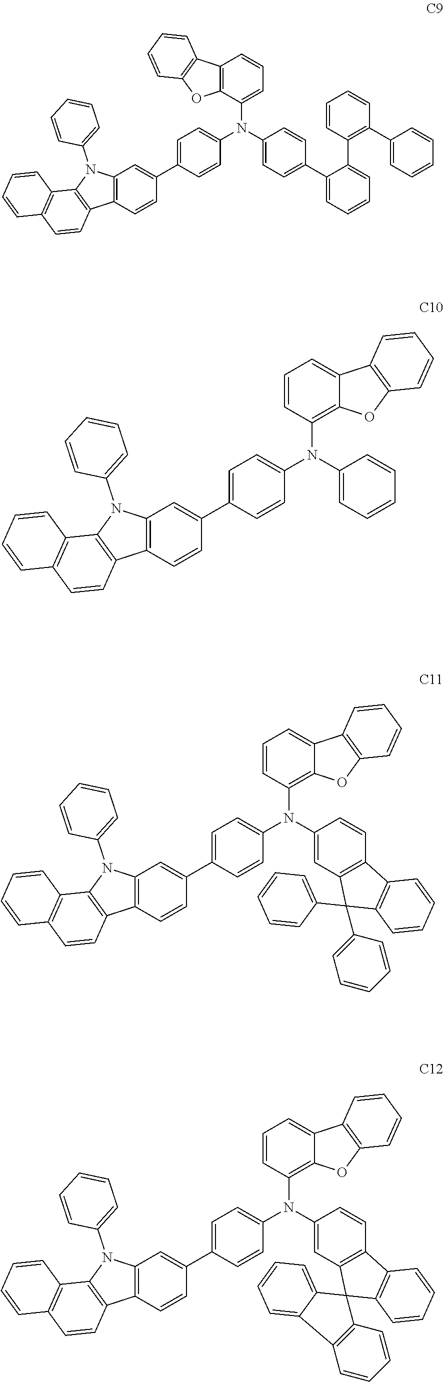 [SMILES: c1ccc(-c2ccccc2-c2ccccc2-c2ccc(N(c3ccc(-c4ccc5c6ccc7ccccc7c6n(-c6ccccc6)c5c4)cc3)c3cccc4c3oc3ccccc34)cc2)cc1 ; c1ccc(N(c2ccc(-c3ccc4c5ccc6ccccc6c5n(-c5ccccc5)c4c3)cc2)c2cccc3c2oc2ccccc23)cc1 ; c1ccc(-n2c3cc(-c4ccc(N(c5ccc6c(c5)C(c5ccccc5)(c5ccccc5)c5ccccc5-6)c5cccc6c5oc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5cccc6c5oc5ccccc56)cc4)ccc3c3ccc4ccccc4c32)cc1] 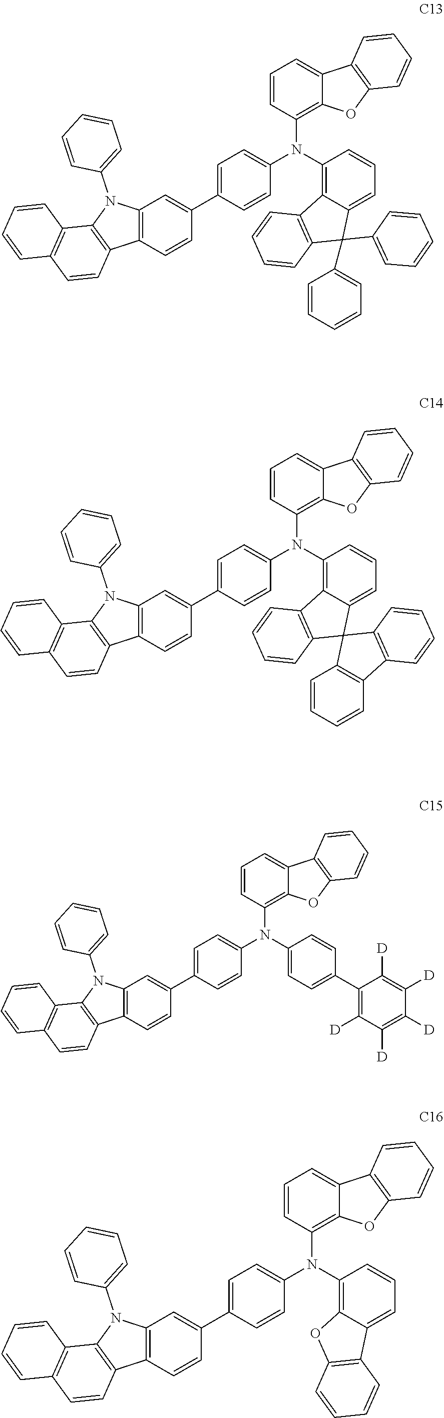 [SMILES: c1ccc(-n2c3cc(-c4ccc(N(c5cccc6c5-c5ccccc5C6(c5ccccc5)c5ccccc5)c5cccc6c5oc5ccccc56)cc4)ccc3c3ccc4ccccc4c32)cc1 ; c1ccc(-n2c3cc(-c4ccc(N(c5cccc6c5-c5ccccc5C65c6ccccc6-c6ccccc65)c5cccc6c5oc5ccccc56)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3cccc4c3oc3ccccc34)cc2)c([2H])c1[2H] ; c1ccc(-n2c3cc(-c4ccc(N(c5cccc6c5oc5ccccc56)c5cccc6c5oc5ccccc56)cc4)ccc3c3ccc4ccccc4c32)cc1] 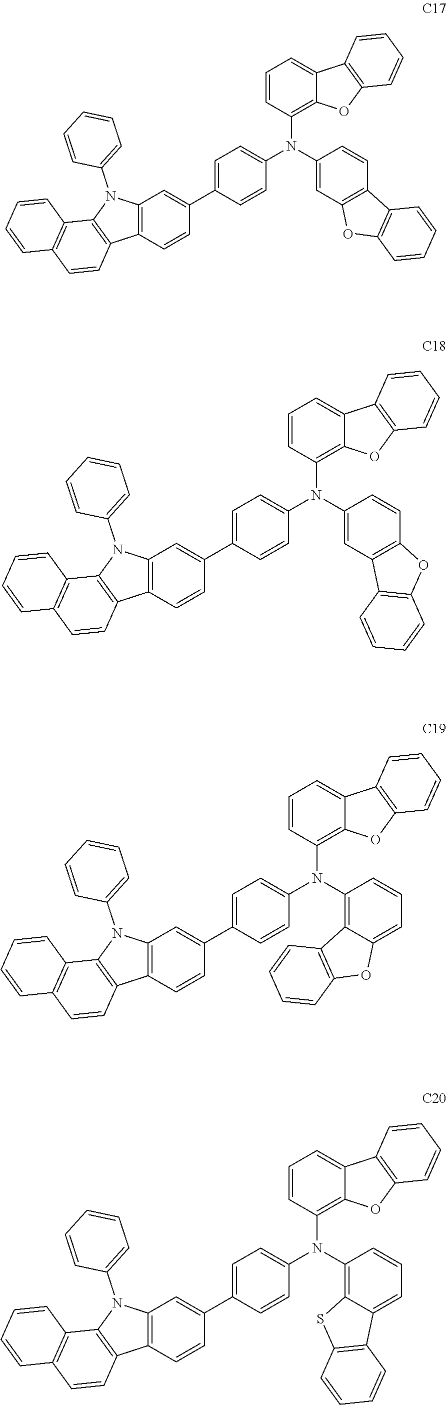 [SMILES: c1ccc(-n2c3cc(-c4ccc(N(c5ccc6c(c5)oc5ccccc56)c5cccc6c5oc5ccccc56)cc4)ccc3c3ccc4ccccc4c32)cc1 ; c1ccc(-n2c3cc(-c4ccc(N(c5ccc6oc7ccccc7c6c5)c5cccc6c5oc5ccccc56)cc4)ccc3c3ccc4ccccc4c32)cc1 ; c1ccc(-n2c3cc(-c4ccc(N(c5cccc6c5oc5ccccc56)c5cccc6oc7ccccc7c56)cc4)ccc3c3ccc4ccccc4c32)cc1 ; c1ccc(-n2c3cc(-c4ccc(N(c5cccc6c5oc5ccccc56)c5cccc6c5sc5ccccc56)cc4)ccc3c3ccc4ccccc4c32)cc1] 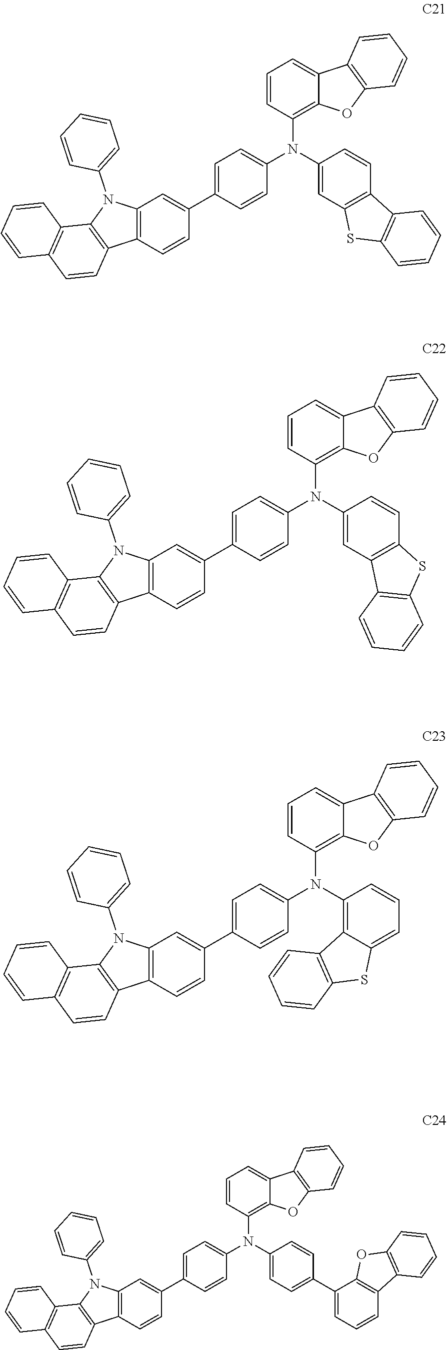 [SMILES: c1ccc(-n2c3cc(-c4ccc(N(c5ccc6c(c5)sc5ccccc56)c5cccc6c5oc5ccccc56)cc4)ccc3c3ccc4ccccc4c32)cc1 ; c1ccc(-n2c3cc(-c4ccc(N(c5ccc6sc7ccccc7c6c5)c5cccc6c5oc5ccccc56)cc4)ccc3c3ccc4ccccc4c32)cc1 ; c1ccc(-n2c3cc(-c4ccc(N(c5cccc6c5oc5ccccc56)c5cccc6sc7ccccc7c56)cc4)ccc3c3ccc4ccccc4c32)cc1 ; c1ccc(-n2c3cc(-c4ccc(N(c5ccc(-c6cccc7c6oc6ccccc67)cc5)c5cccc6c5oc5ccccc56)cc4)ccc3c3ccc4ccccc4c32)cc1] 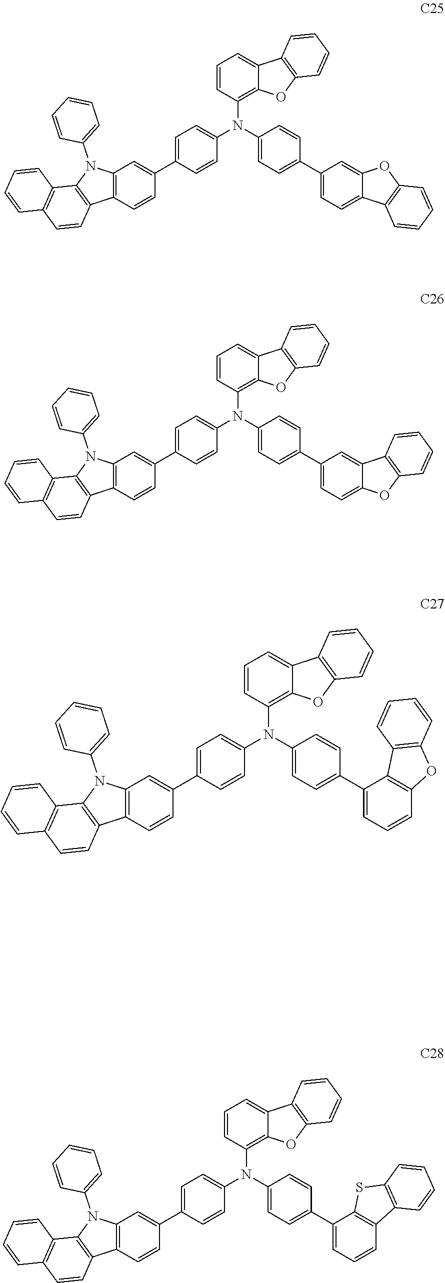 [SMILES: c1ccc(-n2c3cc(-c4ccc(N(c5ccc(-c6ccc7c(c6)oc6ccccc67)cc5)c5cccc6c5oc5ccccc56)cc4)ccc3c3ccc4ccccc4c32)cc1 ; c1ccc(-n2c3cc(-c4ccc(N(c5ccc(-c6ccc7oc8ccccc8c7c6)cc5)c5cccc6c5oc5ccccc56)cc4)ccc3c3ccc4ccccc4c32)cc1 ; c1ccc(-n2c3cc(-c4ccc(N(c5ccc(-c6cccc7oc8ccccc8c67)cc5)c5cccc6c5oc5ccccc56)cc4)ccc3c3ccc4ccccc4c32)cc1 ; c1ccc(-n2c3cc(-c4ccc(N(c5ccc(-c6cccc7c6sc6ccccc67)cc5)c5cccc6c5oc5ccccc56)cc4)ccc3c3ccc4ccccc4c32)cc1] 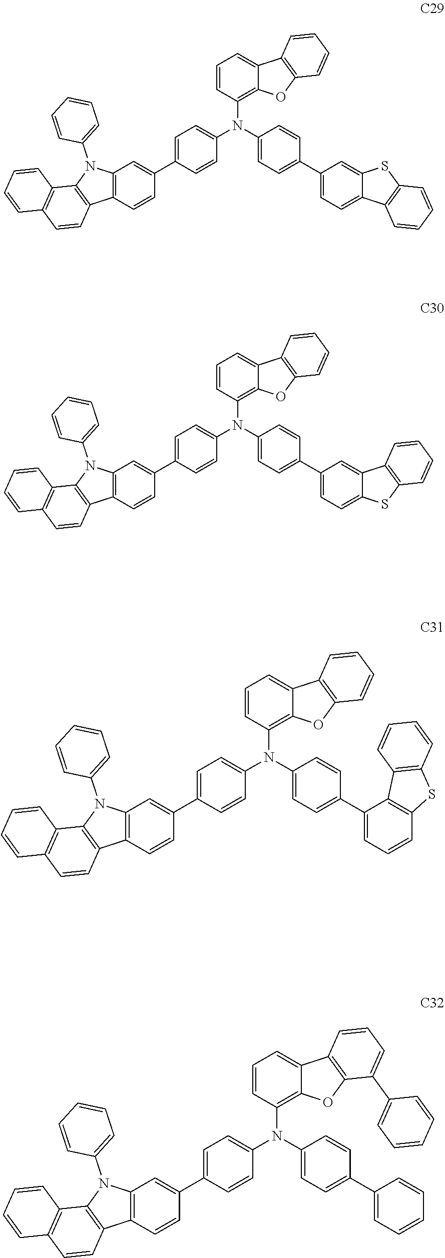 [SMILES: c1ccc(-n2c3cc(-c4ccc(N(c5ccc(-c6ccc7c(c6)sc6ccccc67)cc5)c5cccc6c5oc5ccccc56)cc4)ccc3c3ccc4ccccc4c32)cc1 ; c1ccc(-n2c3cc(-c4ccc(N(c5ccc(-c6ccc7sc8ccccc8c7c6)cc5)c5cccc6c5oc5ccccc56)cc4)ccc3c3ccc4ccccc4c32)cc1 ; c1ccc(-n2c3cc(-c4ccc(N(c5ccc(-c6cccc7sc8ccccc8c67)cc5)c5cccc6c5oc5ccccc56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3cccc4c3oc3c(-c5ccccc5)cccc34)cc2)cc1] 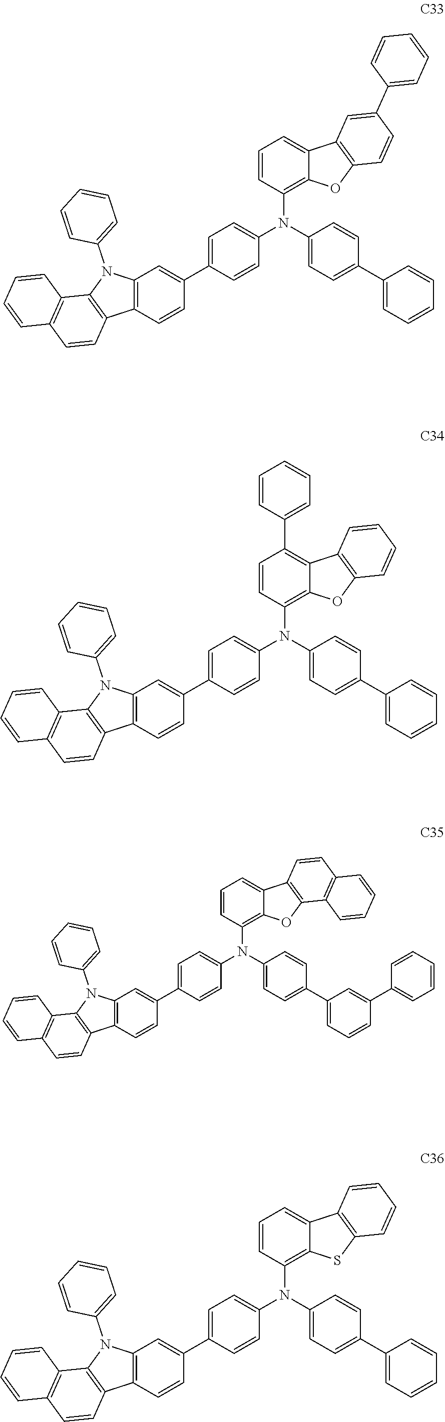 [SMILES: c1ccc(-c2ccc(N(c3ccc(-c4ccc5c6ccc7ccccc7c6n(-c6ccccc6)c5c4)cc3)c3cccc4c3oc3ccc(-c5ccccc5)cc34)cc2)cc1 ; c1ccc(-c2ccc(N(c3ccc(-c4ccc5c6ccc7ccccc7c6n(-c6ccccc6)c5c4)cc3)c3ccc(-c4ccccc4)c4c3oc3ccccc34)cc2)cc1 ; c1ccc(-c2cccc(-c3ccc(N(c4ccc(-c5ccc6c7ccc8ccccc8c7n(-c7ccccc7)c6c5)cc4)c4cccc5c4oc4c6ccccc6ccc54)cc3)c2)cc1 ; c1ccc(-c2ccc(N(c3ccc(-c4ccc5c6ccc7ccccc7c6n(-c6ccccc6)c5c4)cc3)c3cccc4c3sc3ccccc34)cc2)cc1] 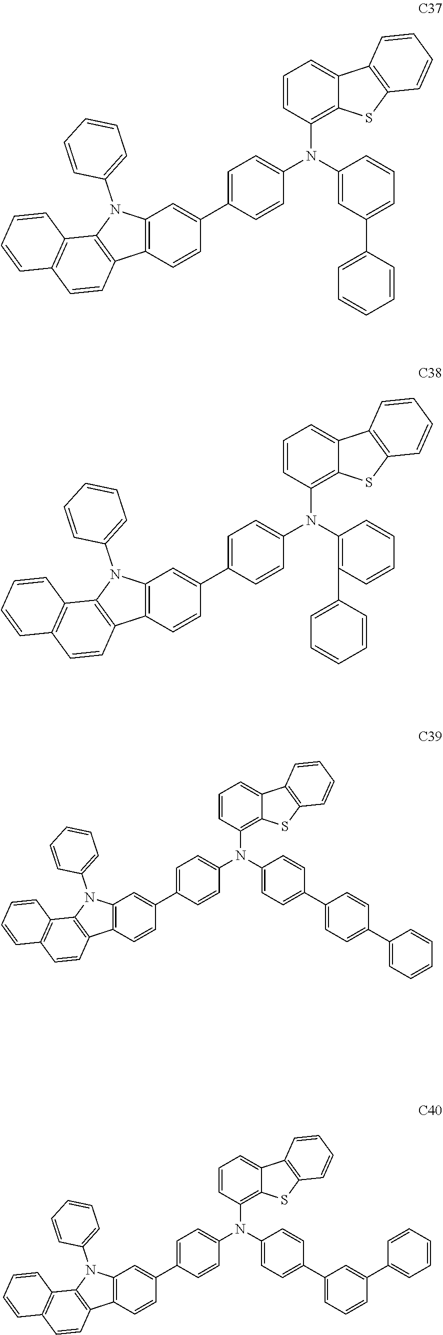 [SMILES: c1ccc(-c2cccc(N(c3ccc(-c4ccc5c6ccc7ccccc7c6n(-c6ccccc6)c5c4)cc3)c3cccc4c3sc3ccccc34)c2)cc1 ; c1ccc(-c2ccccc2N(c2ccc(-c3ccc4c5ccc6ccccc6c5n(-c5ccccc5)c4c3)cc2)c2cccc3c2sc2ccccc23)cc1 ; c1ccc(-c2ccc(-c3ccc(N(c4ccc(-c5ccc6c7ccc8ccccc8c7n(-c7ccccc7)c6c5)cc4)c4cccc5c4sc4ccccc45)cc3)cc2)cc1 ; c1ccc(-c2cccc(-c3ccc(N(c4ccc(-c5ccc6c7ccc8ccccc8c7n(-c7ccccc7)c6c5)cc4)c4cccc5c4sc4ccccc45)cc3)c2)cc1] 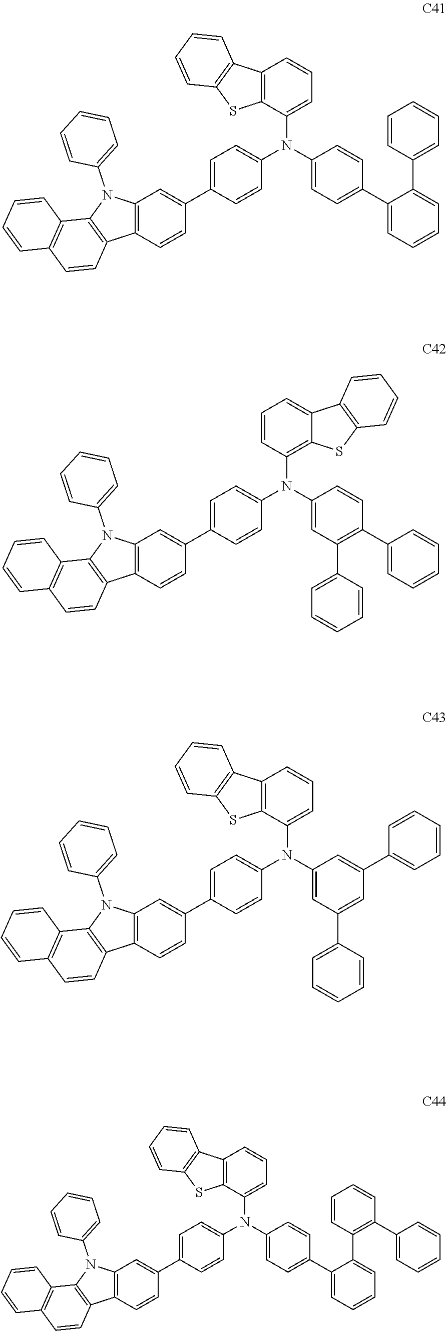 [SMILES: c1ccc(-c2ccccc2-c2ccc(N(c3ccc(-c4ccc5c6ccc7ccccc7c6n(-c6ccccc6)c5c4)cc3)c3cccc4c3sc3ccccc34)cc2)cc1 ; c1ccc(-c2ccc(N(c3ccc(-c4ccc5c6ccc7ccccc7c6n(-c6ccccc6)c5c4)cc3)c3cccc4c3sc3ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4c3sc3ccccc34)c2)cc1 ; c1ccc(-c2ccccc2-c2ccccc2-c2ccc(N(c3ccc(-c4ccc5c6ccc7ccccc7c6n(-c6ccccc6)c5c4)cc3)c3cccc4c3sc3ccccc34)cc2)cc1] 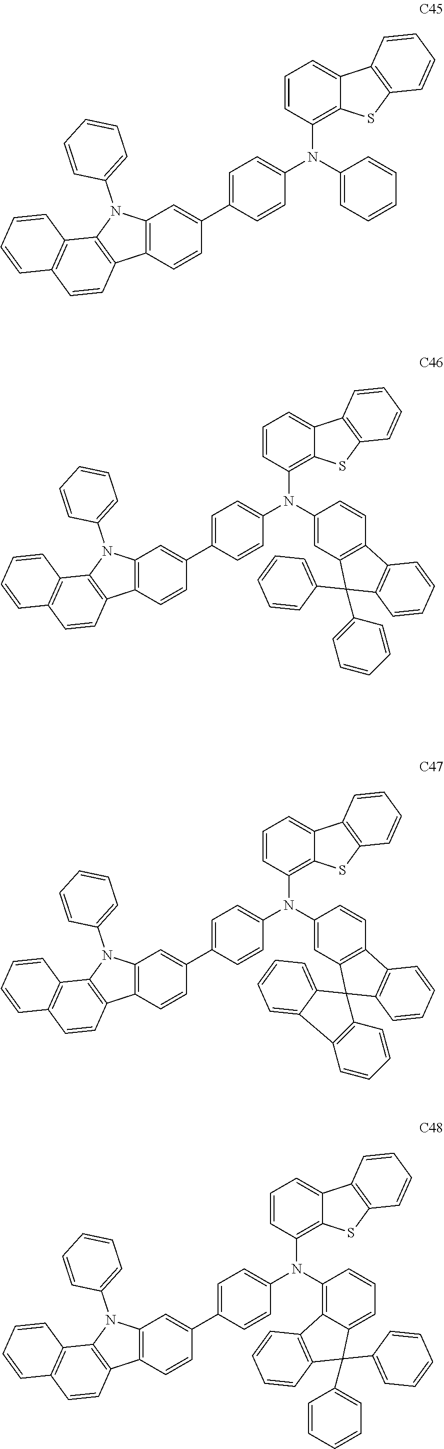 [SMILES: c1ccc(N(c2ccc(-c3ccc4c5ccc6ccccc6c5n(-c5ccccc5)c4c3)cc2)c2cccc3c2sc2ccccc23)cc1 ; c1ccc(-n2c3cc(-c4ccc(N(c5ccc6c(c5)C(c5ccccc5)(c5ccccc5)c5ccccc5-6)c5cccc6c5sc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5cccc6c5sc5ccccc56)cc4)ccc3c3ccc4ccccc4c32)cc1 ; c1ccc(-n2c3cc(-c4ccc(N(c5cccc6c5-c5ccccc5C6(c5ccccc5)c5ccccc5)c5cccc6c5sc5ccccc56)cc4)ccc3c3ccc4ccccc4c32)cc1] 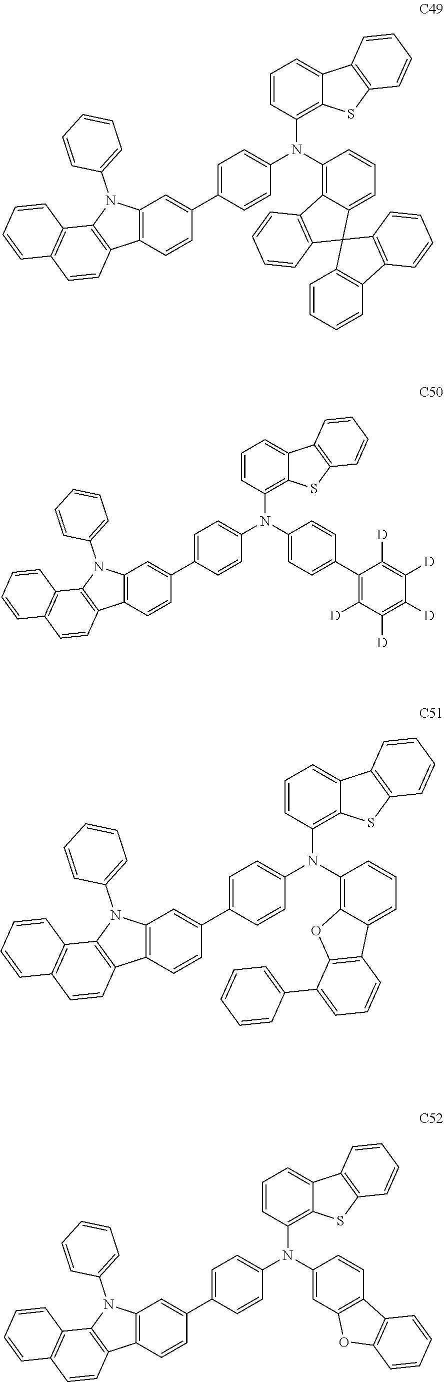 [SMILES: c1ccc(-n2c3cc(-c4ccc(N(c5cccc6c5-c5ccccc5C65c6ccccc6-c6ccccc65)c5cccc6c5sc5ccccc56)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3cccc4c3sc3ccccc34)cc2)c([2H])c1[2H] ; c1ccc(-c2cccc3c2oc2c(N(c4ccc(-c5ccc6c7ccc8ccccc8c7n(-c7ccccc7)c6c5)cc4)c4cccc5c4sc4ccccc45)cccc23)cc1 ; c1ccc(-n2c3cc(-c4ccc(N(c5ccc6c(c5)oc5ccccc56)c5cccc6c5sc5ccccc56)cc4)ccc3c3ccc4ccccc4c32)cc1] 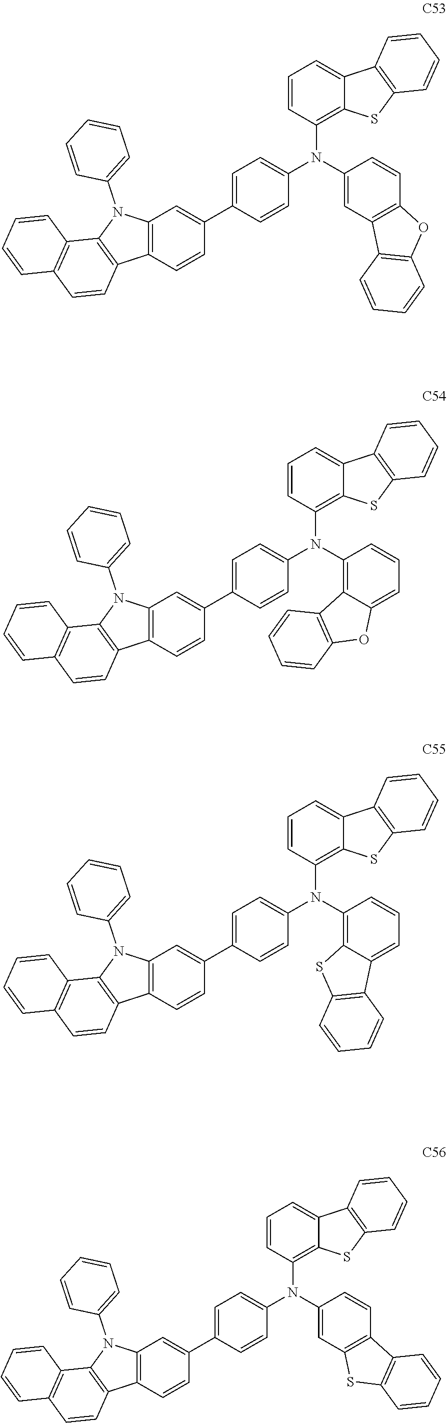 [SMILES: c1ccc(-n2c3cc(-c4ccc(N(c5ccc6oc7ccccc7c6c5)c5cccc6c5sc5ccccc56)cc4)ccc3c3ccc4ccccc4c32)cc1 ; c1ccc(-n2c3cc(-c4ccc(N(c5cccc6c5sc5ccccc56)c5cccc6oc7ccccc7c56)cc4)ccc3c3ccc4ccccc4c32)cc1 ; c1ccc(-n2c3cc(-c4ccc(N(c5cccc6c5sc5ccccc56)c5cccc6c5sc5ccccc56)cc4)ccc3c3ccc4ccccc4c32)cc1 ; c1ccc(-n2c3cc(-c4ccc(N(c5ccc6c(c5)sc5ccccc56)c5cccc6c5sc5ccccc56)cc4)ccc3c3ccc4ccccc4c32)cc1] 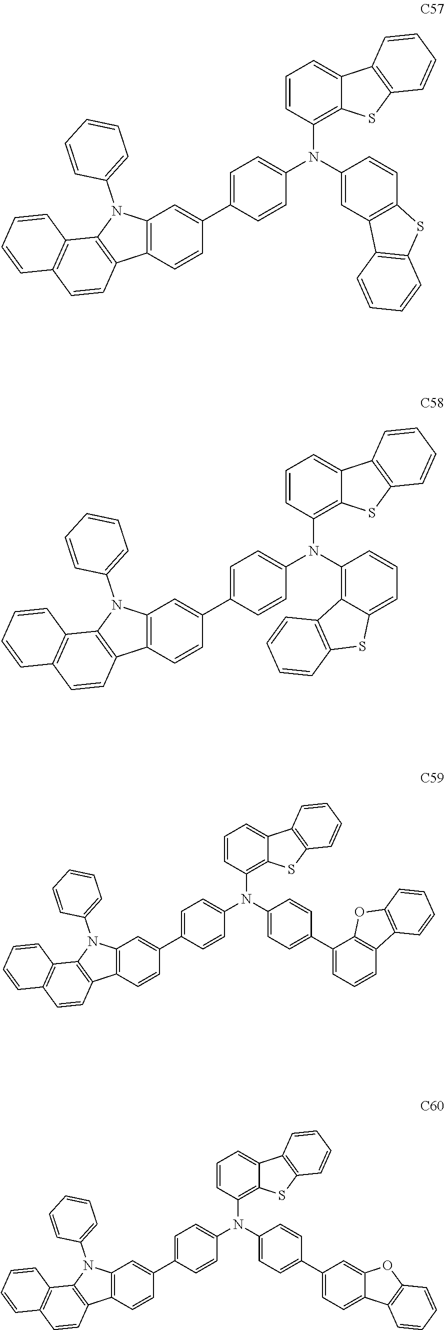 [SMILES: c1ccc(-n2c3cc(-c4ccc(N(c5ccc6sc7ccccc7c6c5)c5cccc6c5sc5ccccc56)cc4)ccc3c3ccc4ccccc4c32)cc1 ; c1ccc(-n2c3cc(-c4ccc(N(c5cccc6c5sc5ccccc56)c5cccc6sc7ccccc7c56)cc4)ccc3c3ccc4ccccc4c32)cc1 ; c1ccc(-n2c3cc(-c4ccc(N(c5ccc(-c6cccc7c6oc6ccccc67)cc5)c5cccc6c5sc5ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5cccc6c5sc5ccccc56)cc4)ccc3c3ccc4ccccc4c32)cc1] 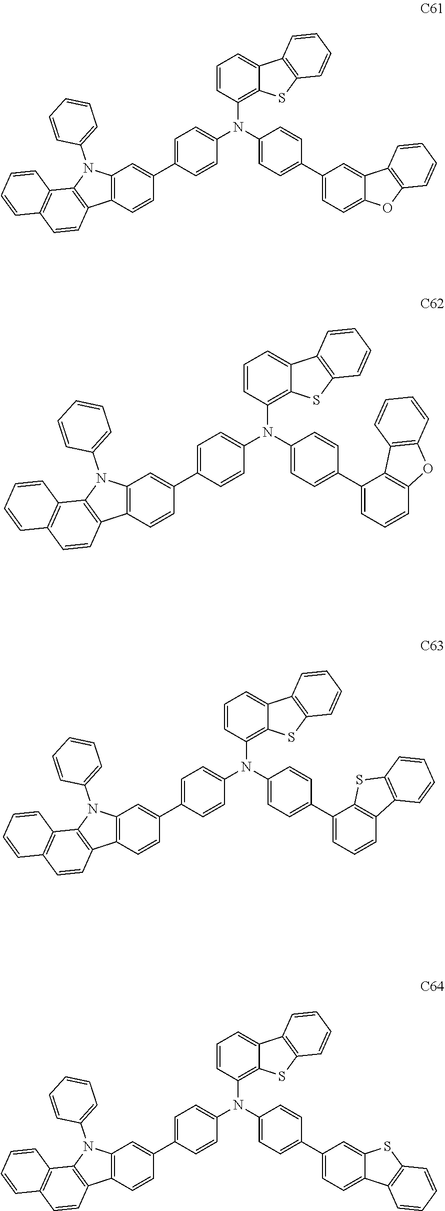 [SMILES: c1ccc(-n2c3cc(-c4ccc(N(c5ccc(-c6ccc7oc8ccccc8c7c6)cc5)c5cccc6c5sc5ccccc56)cc4)ccc3c3ccc4ccccc4c32)cc1 ; c1ccc(-n2c3cc(-c4ccc(N(c5ccc(-c6cccc7oc8ccccc8c67)cc5)c5cccc6c5sc5ccccc56)cc4)ccc3c3ccc4ccccc4c32)cc1 ; c1ccc(-n2c3cc(-c4ccc(N(c5ccc(-c6cccc7c6sc6ccccc67)cc5)c5cccc6c5sc5ccccc56)cc4)ccc3c3ccc4ccccc4c32)cc1 ; c1ccc(-n2c3cc(-c4ccc(N(c5ccc(-c6ccc7c(c6)sc6ccccc67)cc5)c5cccc6c5sc5ccccc56)cc4)ccc3c3ccc4ccccc4c32)cc1] 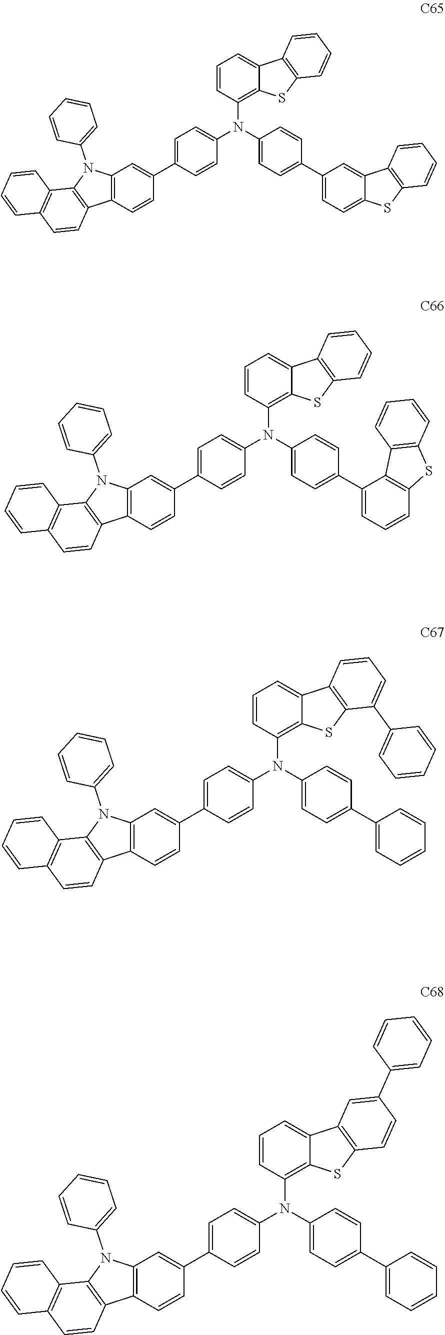 [SMILES: c1ccc(-n2c3cc(-c4ccc(N(c5ccc(-c6ccc7sc8ccccc8c7c6)cc5)c5cccc6c5sc5ccccc56)cc4)ccc3c3ccc4ccccc4c32)cc1 ; c1ccc(-n2c3cc(-c4ccc(N(c5ccc(-c6cccc7sc8ccccc8c67)cc5)c5cccc6c5sc5ccccc56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3cccc4c3sc3c(-c5ccccc5)cccc34)cc2)cc1 ; c1ccc(-c2ccc(N(c3ccc(-c4ccc5c6ccc7ccccc7c6n(-c6ccccc6)c5c4)cc3)c3cccc4c3sc3ccc(-c5ccccc5)cc34)cc2)cc1] 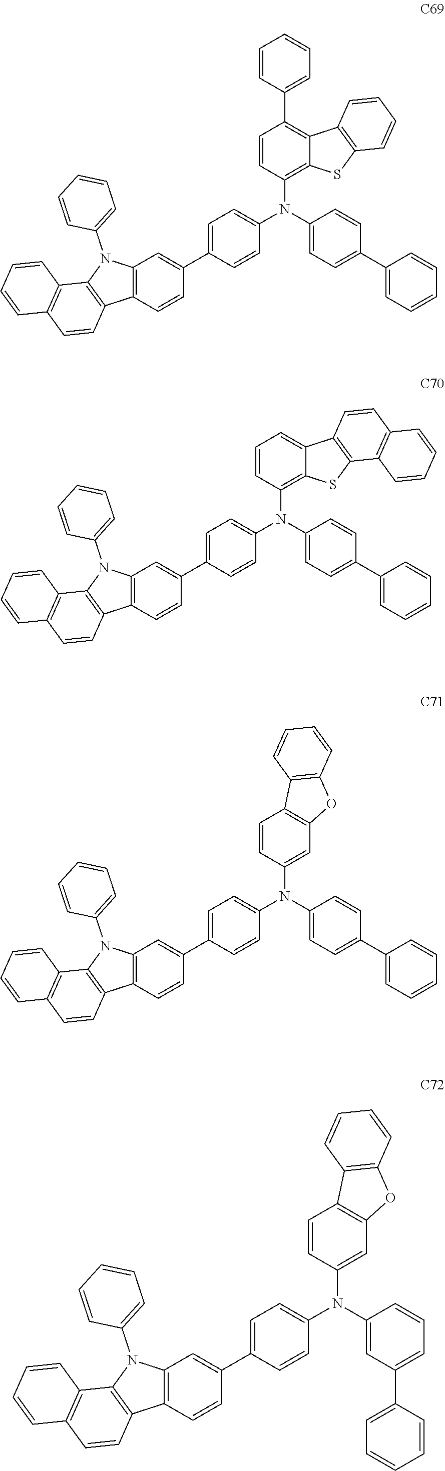 [SMILES: c1ccc(-c2ccc(N(c3ccc(-c4ccc5c6ccc7ccccc7c6n(-c6ccccc6)c5c4)cc3)c3ccc(-c4ccccc4)c4c3sc3ccccc34)cc2)cc1 ; c1ccc(-c2ccc(N(c3ccc(-c4ccc5c6ccc7ccccc7c6n(-c6ccccc6)c5c4)cc3)c3cccc4c3sc3c5ccccc5ccc43)cc2)cc1 ; c1ccc(-c2ccc(N(c3ccc(-c4ccc5c6ccc7ccccc7c6n(-c6ccccc6)c5c4)cc3)c3ccc4c(c3)oc3ccccc34)cc2)cc1 ; c1ccc(-c2cccc(N(c3ccc(-c4ccc5c6ccc7ccccc7c6n(-c6ccccc6)c5c4)cc3)c3ccc4c(c3)oc3ccccc34)c2)cc1] 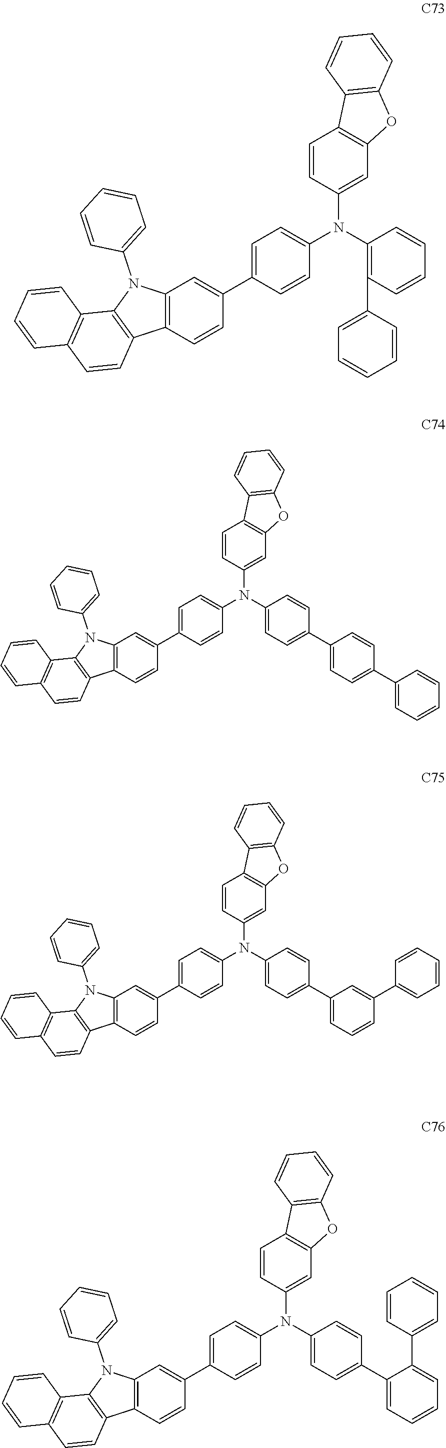 [SMILES: c1ccc(-c2ccccc2N(c2ccc(-c3ccc4c5ccc6ccccc6c5n(-c5ccccc5)c4c3)cc2)c2ccc3c(c2)oc2ccccc23)cc1 ; c1ccc(-c2ccc(-c3ccc(N(c4ccc(-c5ccc6c7ccc8ccccc8c7n(-c7ccccc7)c6c5)cc4)c4ccc5c(c4)oc4ccccc45)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)oc4ccccc45)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(c3)oc3ccccc34)cc2)cc1] 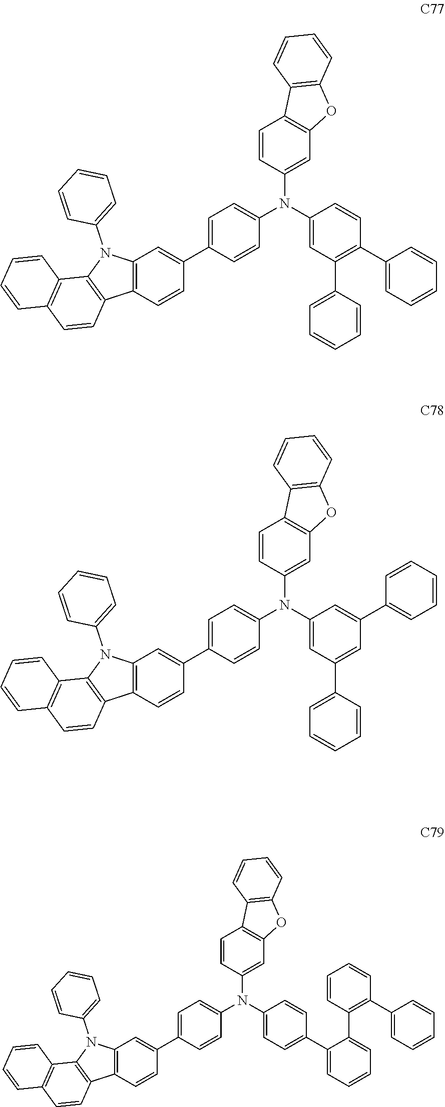 [SMILES: c1ccc(-c2ccc(N(c3ccc(-c4ccc5c6ccc7ccccc7c6n(-c6ccccc6)c5c4)cc3)c3ccc4c(c3)oc3ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(c3)oc3ccccc34)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)oc3ccccc34)cc2)cc1] 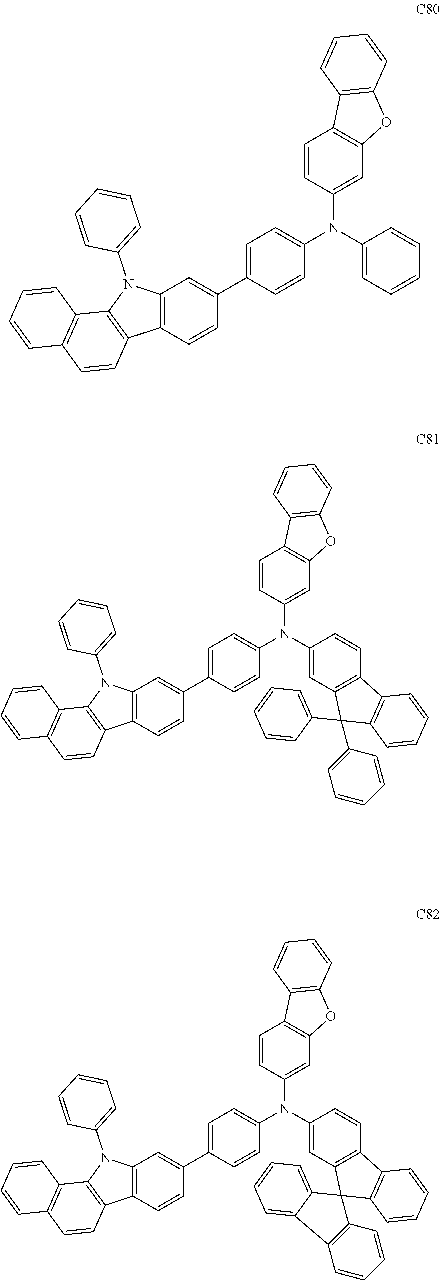 [SMILES: c1ccc(N(c2ccc(-c3ccc4c5ccc6ccccc6c5n(-c5ccccc5)c4c3)cc2)c2ccc3c(c2)oc2ccccc23)cc1 ; c1ccc(-n2c3cc(-c4ccc(N(c5ccc6c(c5)C(c5ccccc5)(c5ccccc5)c5ccccc5-6)c5ccc6c(c5)oc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6c(c5)oc5ccccc56)cc4)ccc3c3ccc4ccccc4c32)cc1] 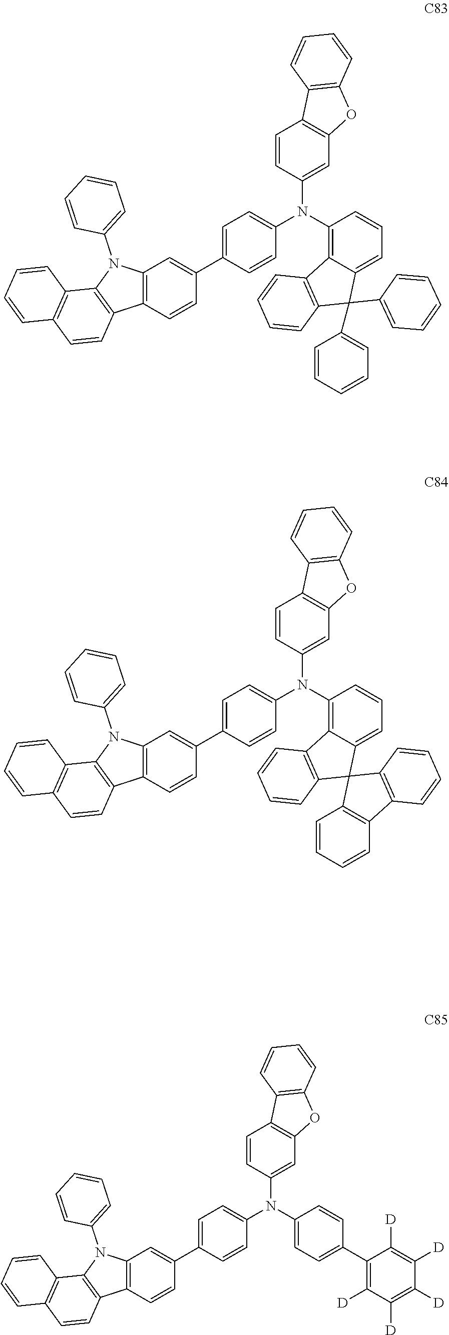 [SMILES: c1ccc(-n2c3cc(-c4ccc(N(c5ccc6c(c5)oc5ccccc56)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc6c(c5)oc5ccccc56)c5cccc6c5-c5ccccc5C65c6ccccc6-c6ccccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4c(c3)oc3ccccc34)cc2)c([2H])c1[2H]] 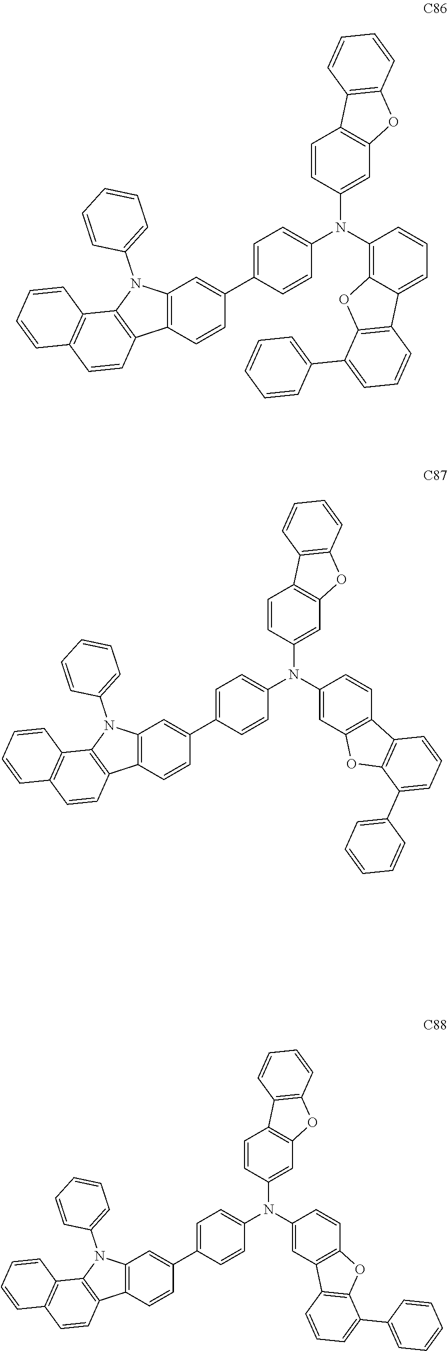 [SMILES: c1ccc(-c2cccc3c2oc2c(N(c4ccc(-c5ccc6c7ccc8ccccc8c7n(-c7ccccc7)c6c5)cc4)c4ccc5c(c4)oc4ccccc45)cccc23)cc1 ; c1ccc(-c2cccc3c2oc2cc(N(c4ccc(-c5ccc6c7ccc8ccccc8c7n(-c7ccccc7)c6c5)cc4)c4ccc5c(c4)oc4ccccc45)ccc23)cc1 ; c1ccc(-c2cccc3c2oc2ccc(N(c4ccc(-c5ccc6c7ccc8ccccc8c7n(-c7ccccc7)c6c5)cc4)c4ccc5c(c4)oc4ccccc45)cc23)cc1] 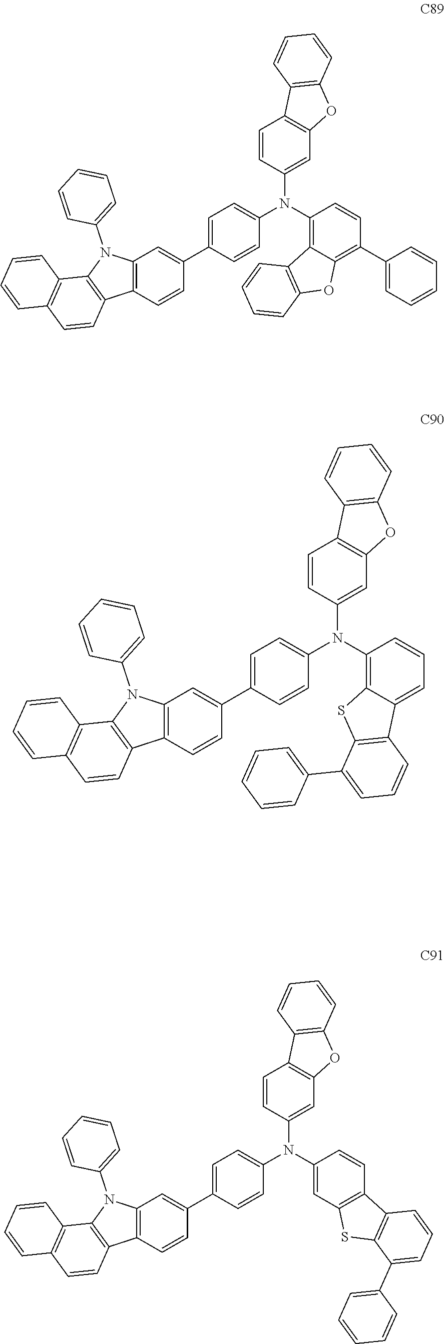 [SMILES: c1ccc(-c2ccc(N(c3ccc(-c4ccc5c6ccc7ccccc7c6n(-c6ccccc6)c5c4)cc3)c3ccc4c(c3)oc3ccccc34)c3c2oc2ccccc23)cc1 ; c1ccc(-c2cccc3c2sc2c(N(c4ccc(-c5ccc6c7ccc8ccccc8c7n(-c7ccccc7)c6c5)cc4)c4ccc5c(c4)oc4ccccc45)cccc23)cc1 ; c1ccc(-c2cccc3c2sc2cc(N(c4ccc(-c5ccc6c7ccc8ccccc8c7n(-c7ccccc7)c6c5)cc4)c4ccc5c(c4)oc4ccccc45)ccc23)cc1] 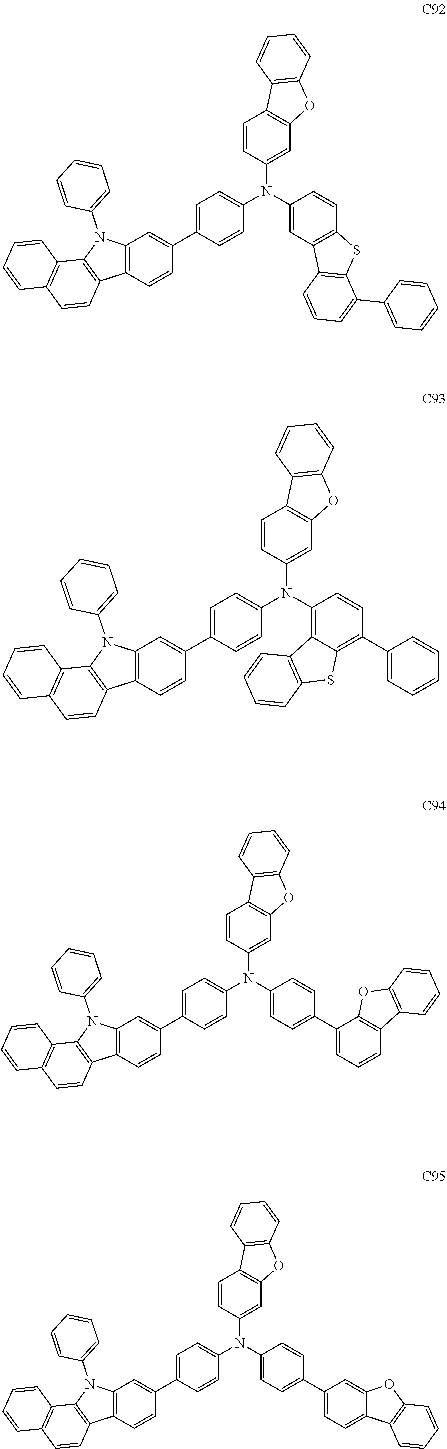 [SMILES: c1ccc(-c2cccc3c2sc2ccc(N(c4ccc(-c5ccc6c7ccc8ccccc8c7n(-c7ccccc7)c6c5)cc4)c4ccc5c(c4)oc4ccccc45)cc23)cc1 ; c1ccc(-c2ccc(N(c3ccc(-c4ccc5c6ccc7ccccc7c6n(-c6ccccc6)c5c4)cc3)c3ccc4c(c3)oc3ccccc34)c3c2sc2ccccc23)cc1 ; c1ccc(-n2c3cc(-c4ccc(N(c5ccc(-c6cccc7c6oc6ccccc67)cc5)c5ccc6c(c5)oc5ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5ccc6c(c5)oc5ccccc56)cc4)ccc3c3ccc4ccccc4c32)cc1] 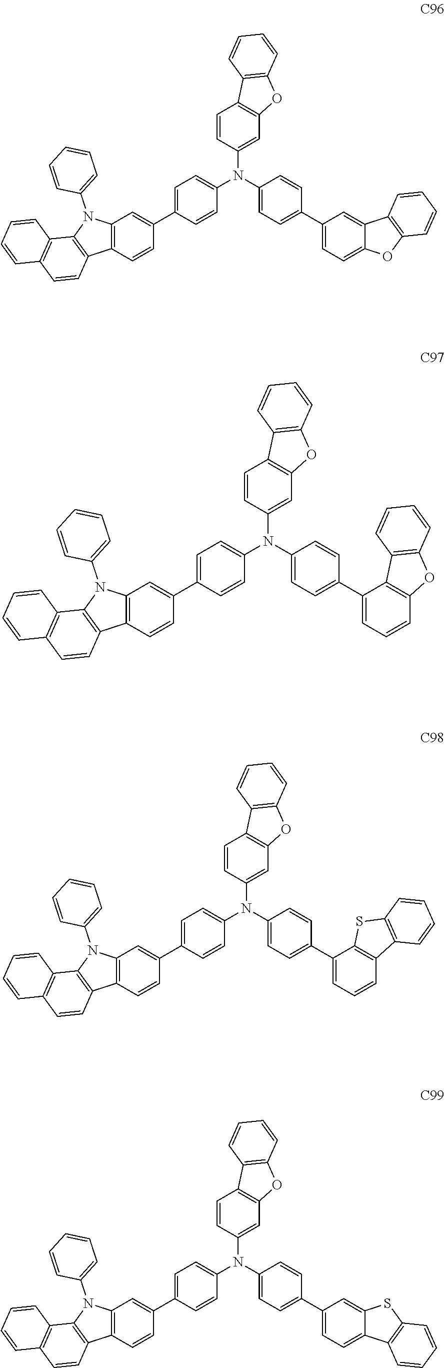 [SMILES: c1ccc(-n2c3cc(-c4ccc(N(c5ccc(-c6ccc7oc8ccccc8c7c6)cc5)c5ccc6c(c5)oc5ccccc56)cc4)ccc3c3ccc4ccccc4c32)cc1 ; c1ccc(-n2c3cc(-c4ccc(N(c5ccc(-c6cccc7oc8ccccc8c67)cc5)c5ccc6c(c5)oc5ccccc56)cc4)ccc3c3ccc4ccccc4c32)cc1 ; c1ccc(-n2c3cc(-c4ccc(N(c5ccc(-c6cccc7c6sc6ccccc67)cc5)c5ccc6c(c5)oc5ccccc56)cc4)ccc3c3ccc4ccccc4c32)cc1 ; c1ccc(-n2c3cc(-c4ccc(N(c5ccc(-c6ccc7c(c6)sc6ccccc67)cc5)c5ccc6c(c5)oc5ccccc56)cc4)ccc3c3ccc4ccccc4c32)cc1] 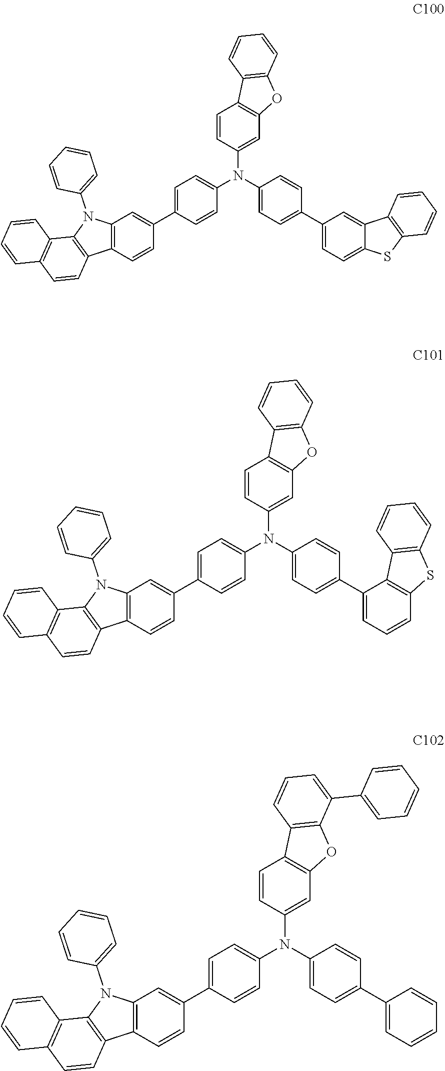 [SMILES: c1ccc(-n2c3cc(-c4ccc(N(c5ccc(-c6ccc7sc8ccccc8c7c6)cc5)c5ccc6c(c5)oc5ccccc56)cc4)ccc3c3ccc4ccccc4c32)cc1 ; c1ccc(-n2c3cc(-c4ccc(N(c5ccc(-c6cccc7sc8ccccc8c67)cc5)c5ccc6c(c5)oc5ccccc56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4c(c3)oc3c(-c5ccccc5)cccc34)cc2)cc1] 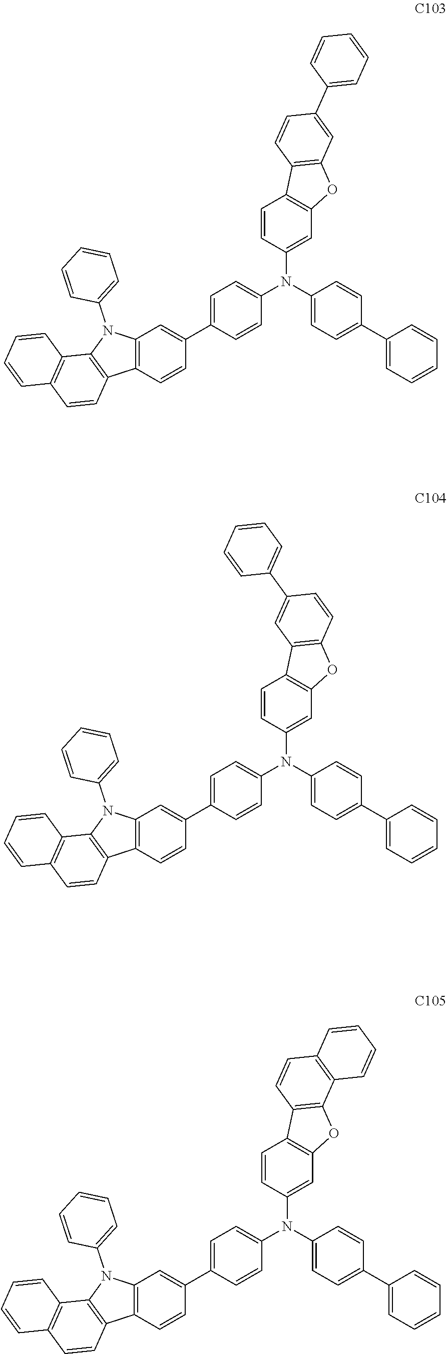 [SMILES: c1ccc(-c2ccc(N(c3ccc(-c4ccc5c6ccc7ccccc7c6n(-c6ccccc6)c5c4)cc3)c3ccc4c(c3)oc3cc(-c5ccccc5)ccc34)cc2)cc1 ; c1ccc(-c2ccc(N(c3ccc(-c4ccc5c6ccc7ccccc7c6n(-c6ccccc6)c5c4)cc3)c3ccc4c(c3)oc3ccc(-c5ccccc5)cc34)cc2)cc1 ; c1ccc(-c2ccc(N(c3ccc(-c4ccc5c6ccc7ccccc7c6n(-c6ccccc6)c5c4)cc3)c3ccc4c(c3)oc3c5ccccc5ccc43)cc2)cc1] 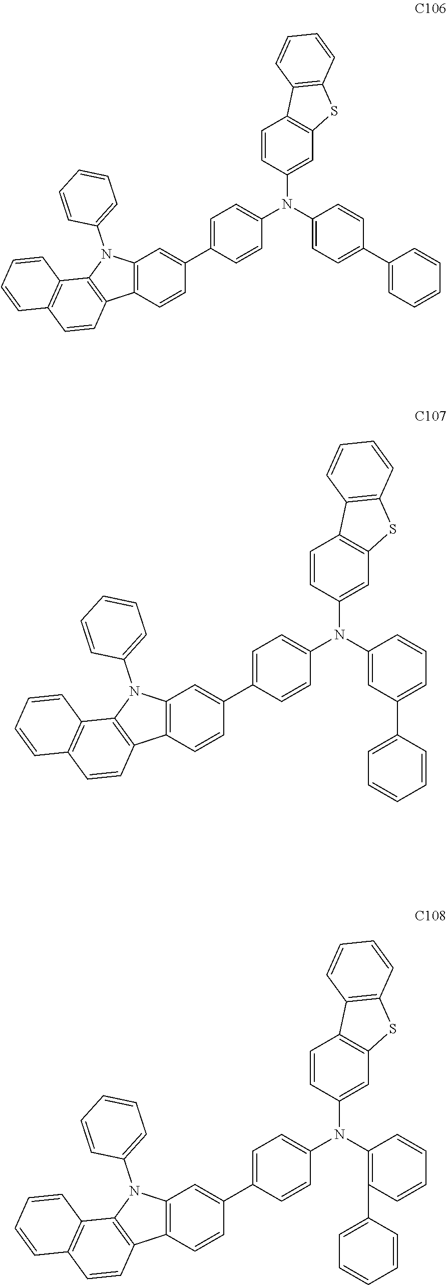 [SMILES: c1ccc(-c2ccc(N(c3ccc(-c4ccc5c6ccc7ccccc7c6n(-c6ccccc6)c5c4)cc3)c3ccc4c(c3)sc3ccccc34)cc2)cc1 ; c1ccc(-c2cccc(N(c3ccc(-c4ccc5c6ccc7ccccc7c6n(-c6ccccc6)c5c4)cc3)c3ccc4c(c3)sc3ccccc34)c2)cc1 ; c1ccc(-c2ccccc2N(c2ccc(-c3ccc4c5ccc6ccccc6c5n(-c5ccccc5)c4c3)cc2)c2ccc3c(c2)sc2ccccc23)cc1] 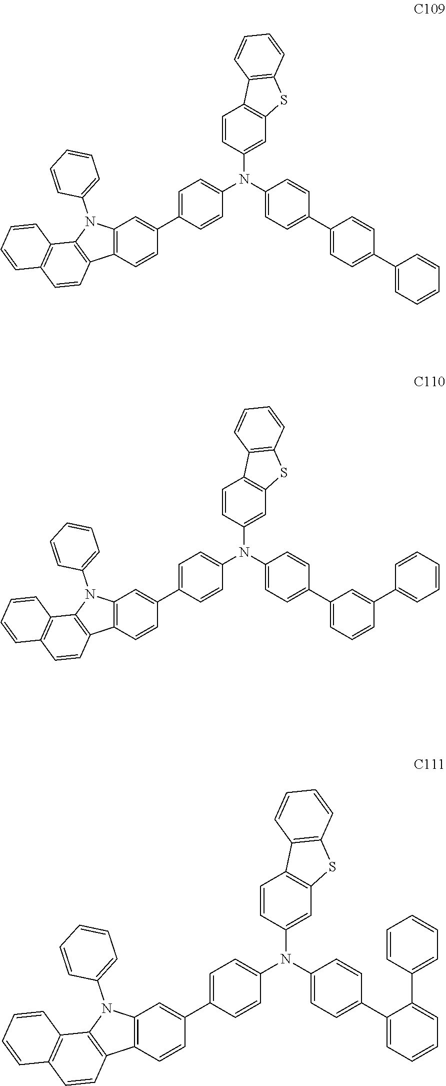 [SMILES: c1ccc(-c2ccc(-c3ccc(N(c4ccc(-c5ccc6c7ccc8ccccc8c7n(-c7ccccc7)c6c5)cc4)c4ccc5c(c4)sc4ccccc45)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)sc4ccccc45)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(c3)sc3ccccc34)cc2)cc1] 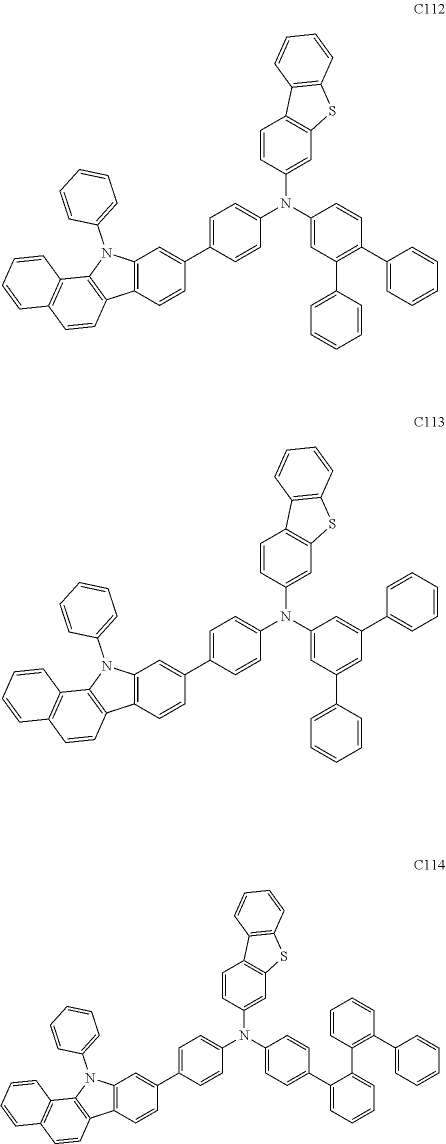 [SMILES: c1ccc(-c2ccc(N(c3ccc(-c4ccc5c6ccc7ccccc7c6n(-c6ccccc6)c5c4)cc3)c3ccc4c(c3)sc3ccccc34)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(c3)sc3ccccc34)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)sc3ccccc34)cc2)cc1] 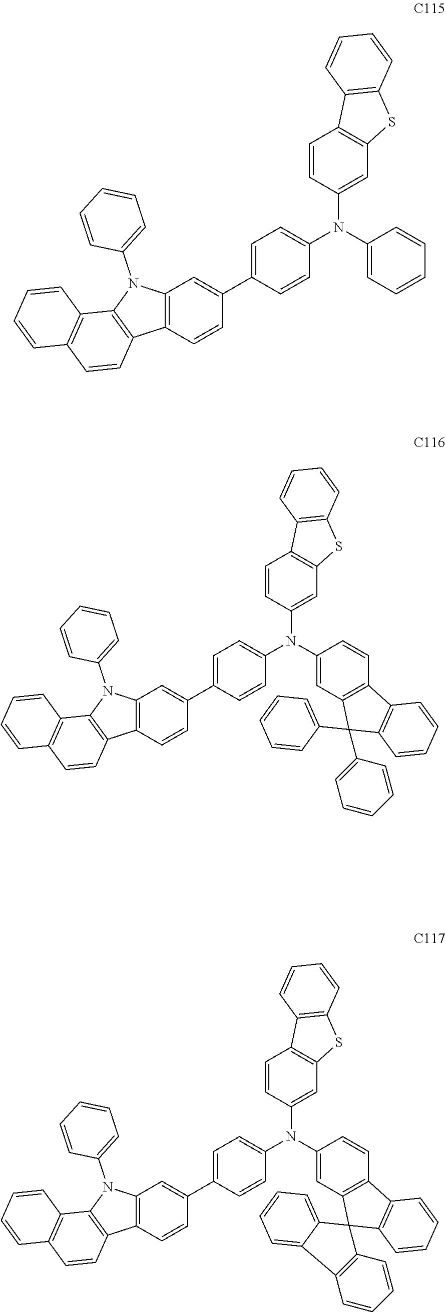 [SMILES: c1ccc(N(c2ccc(-c3ccc4c5ccc6ccccc6c5n(-c5ccccc5)c4c3)cc2)c2ccc3c(c2)sc2ccccc23)cc1 ; c1ccc(-n2c3cc(-c4ccc(N(c5ccc6c(c5)C(c5ccccc5)(c5ccccc5)c5ccccc5-6)c5ccc6c(c5)sc5ccccc56)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6c(c5)sc5ccccc56)cc4)ccc3c3ccc4ccccc4c32)cc1] 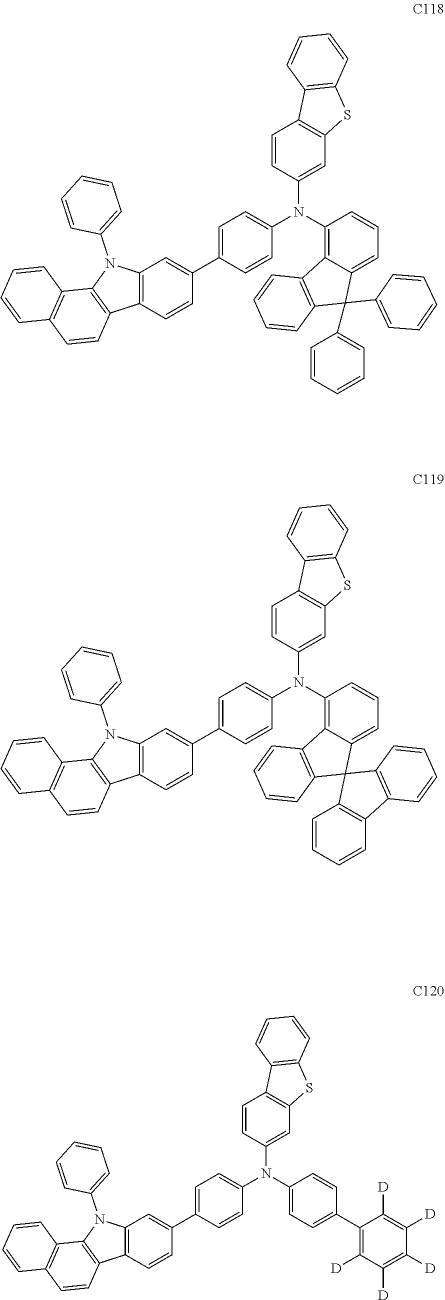 [SMILES: c1ccc(-n2c3cc(-c4ccc(N(c5ccc6c(c5)sc5ccccc56)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc6c(c5)sc5ccccc56)c5cccc6c5-c5ccccc5C65c6ccccc6-c6ccccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4c(c3)sc3ccccc34)cc2)c([2H])c1[2H]] 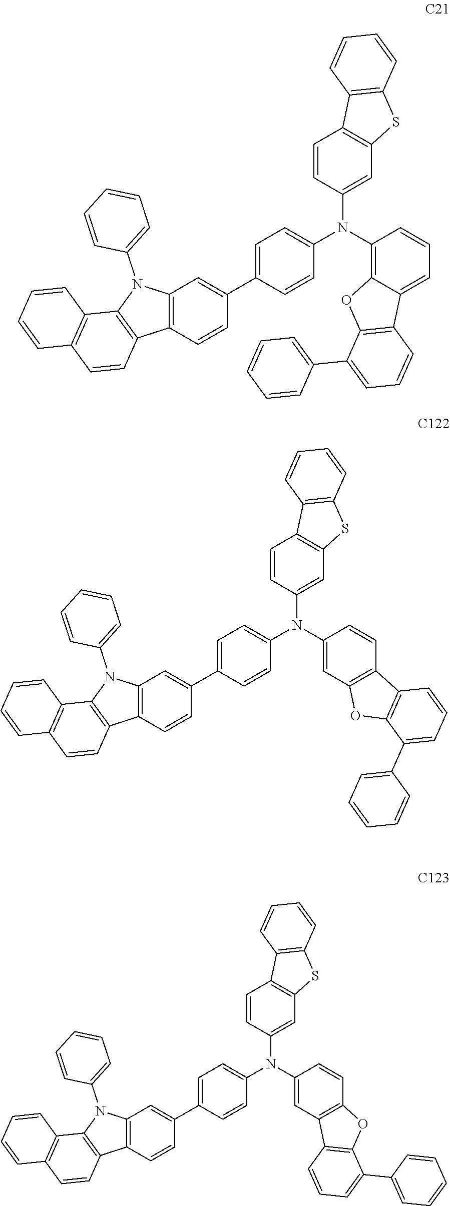 [SMILES: c1ccc(-c2cccc3c2oc2c(N(c4ccc(-c5ccc6c7ccc8ccccc8c7n(-c7ccccc7)c6c5)cc4)c4ccc5c(c4)sc4ccccc45)cccc23)cc1 ; c1ccc(-c2cccc3c2oc2cc(N(c4ccc(-c5ccc6c7ccc8ccccc8c7n(-c7ccccc7)c6c5)cc4)c4ccc5c(c4)sc4ccccc45)ccc23)cc1 ; c1ccc(-c2cccc3c2oc2ccc(N(c4ccc(-c5ccc6c7ccc8ccccc8c7n(-c7ccccc7)c6c5)cc4)c4ccc5c(c4)sc4ccccc45)cc23)cc1] 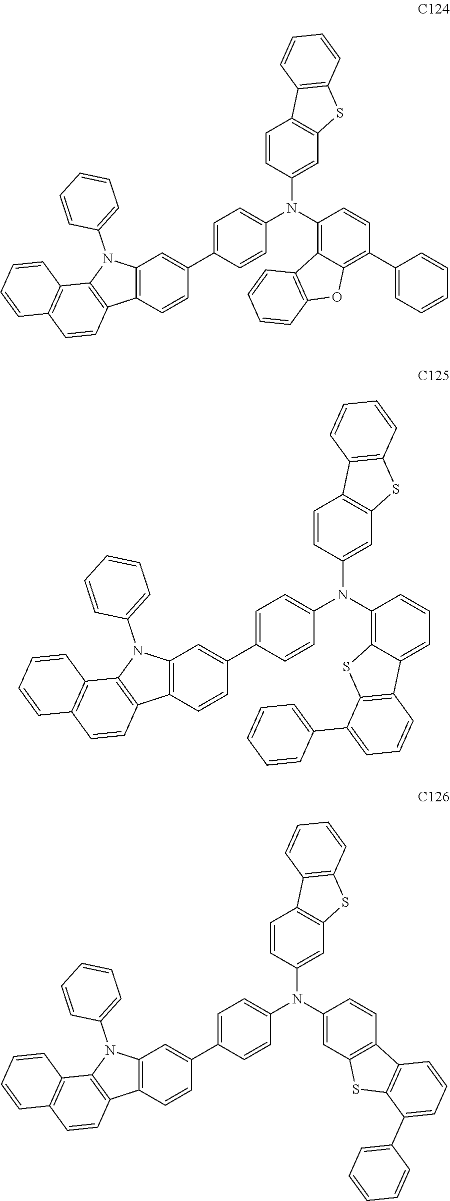 [SMILES: c1ccc(-c2ccc(N(c3ccc(-c4ccc5c6ccc7ccccc7c6n(-c6ccccc6)c5c4)cc3)c3ccc4c(c3)sc3ccccc34)c3c2oc2ccccc23)cc1 ; c1ccc(-c2cccc3c2sc2c(N(c4ccc(-c5ccc6c7ccc8ccccc8c7n(-c7ccccc7)c6c5)cc4)c4ccc5c(c4)sc4ccccc45)cccc23)cc1 ; c1ccc(-c2cccc3c2sc2cc(N(c4ccc(-c5ccc6c7ccc8ccccc8c7n(-c7ccccc7)c6c5)cc4)c4ccc5c(c4)sc4ccccc45)ccc23)cc1] 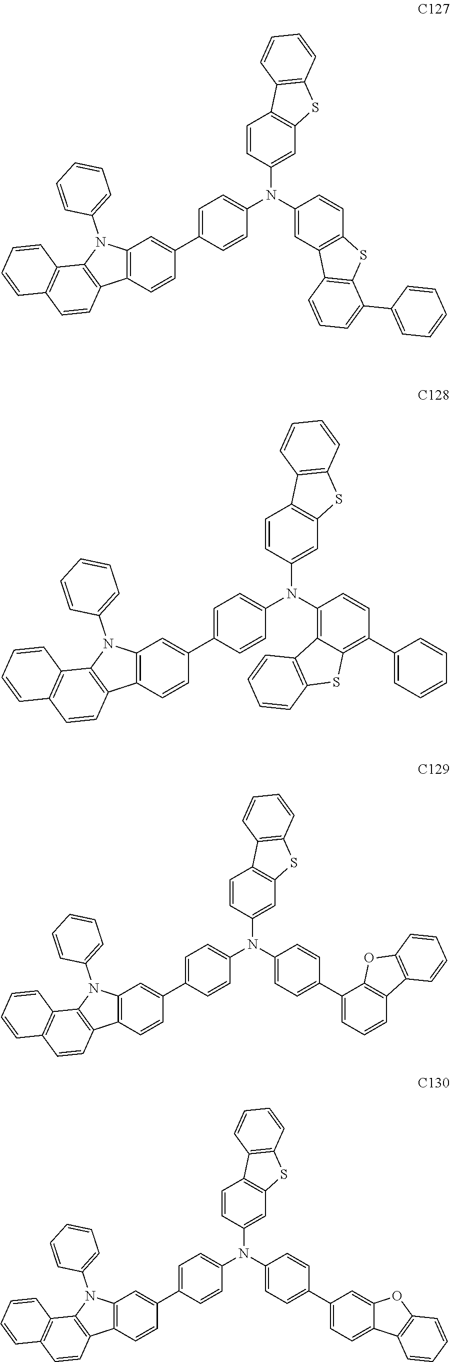 [SMILES: c1ccc(-c2cccc3c2sc2ccc(N(c4ccc(-c5ccc6c7ccc8ccccc8c7n(-c7ccccc7)c6c5)cc4)c4ccc5c(c4)sc4ccccc45)cc23)cc1 ; c1ccc(-c2ccc(N(c3ccc(-c4ccc5c6ccc7ccccc7c6n(-c6ccccc6)c5c4)cc3)c3ccc4c(c3)sc3ccccc34)c3c2sc2ccccc23)cc1 ; c1ccc(-n2c3cc(-c4ccc(N(c5ccc(-c6cccc7c6oc6ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5ccc6c(c5)sc5ccccc56)cc4)ccc3c3ccc4ccccc4c32)cc1] 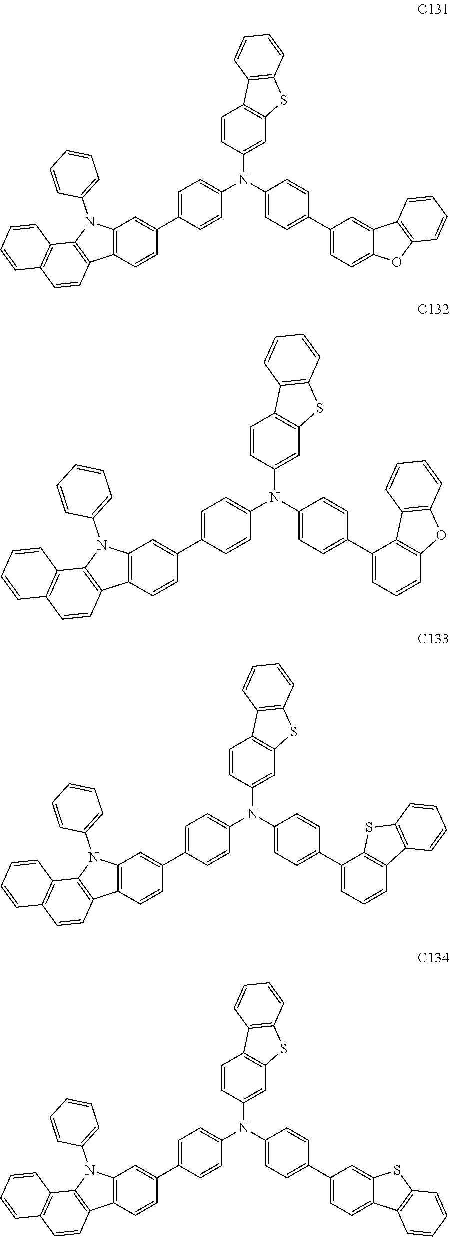 [SMILES: c1ccc(-n2c3cc(-c4ccc(N(c5ccc(-c6ccc7oc8ccccc8c7c6)cc5)c5ccc6c(c5)sc5ccccc56)cc4)ccc3c3ccc4ccccc4c32)cc1 ; c1ccc(-n2c3cc(-c4ccc(N(c5ccc(-c6cccc7oc8ccccc8c67)cc5)c5ccc6c(c5)sc5ccccc56)cc4)ccc3c3ccc4ccccc4c32)cc1 ; c1ccc(-n2c3cc(-c4ccc(N(c5ccc(-c6cccc7c6sc6ccccc67)cc5)c5ccc6c(c5)sc5ccccc56)cc4)ccc3c3ccc4ccccc4c32)cc1 ; c1ccc(-n2c3cc(-c4ccc(N(c5ccc(-c6ccc7c(c6)sc6ccccc67)cc5)c5ccc6c(c5)sc5ccccc56)cc4)ccc3c3ccc4ccccc4c32)cc1] 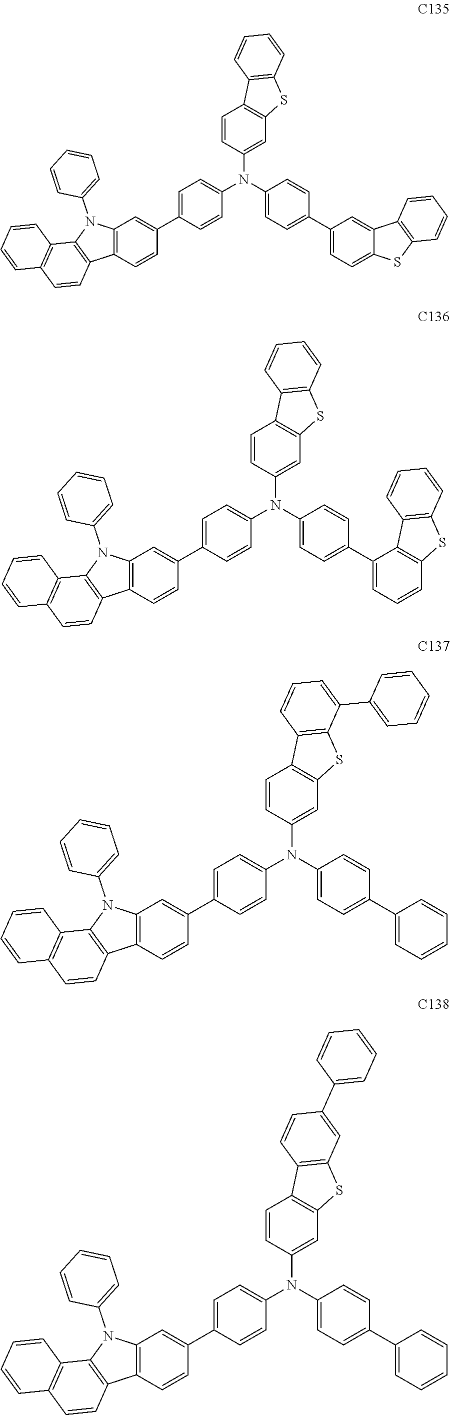 [SMILES: c1ccc(-n2c3cc(-c4ccc(N(c5ccc(-c6ccc7sc8ccccc8c7c6)cc5)c5ccc6c(c5)sc5ccccc56)cc4)ccc3c3ccc4ccccc4c32)cc1 ; c1ccc(-n2c3cc(-c4ccc(N(c5ccc(-c6cccc7sc8ccccc8c67)cc5)c5ccc6c(c5)sc5ccccc56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4c(c3)sc3c(-c5ccccc5)cccc34)cc2)cc1 ; c1ccc(-c2ccc(N(c3ccc(-c4ccc5c6ccc7ccccc7c6n(-c6ccccc6)c5c4)cc3)c3ccc4c(c3)sc3cc(-c5ccccc5)ccc34)cc2)cc1] 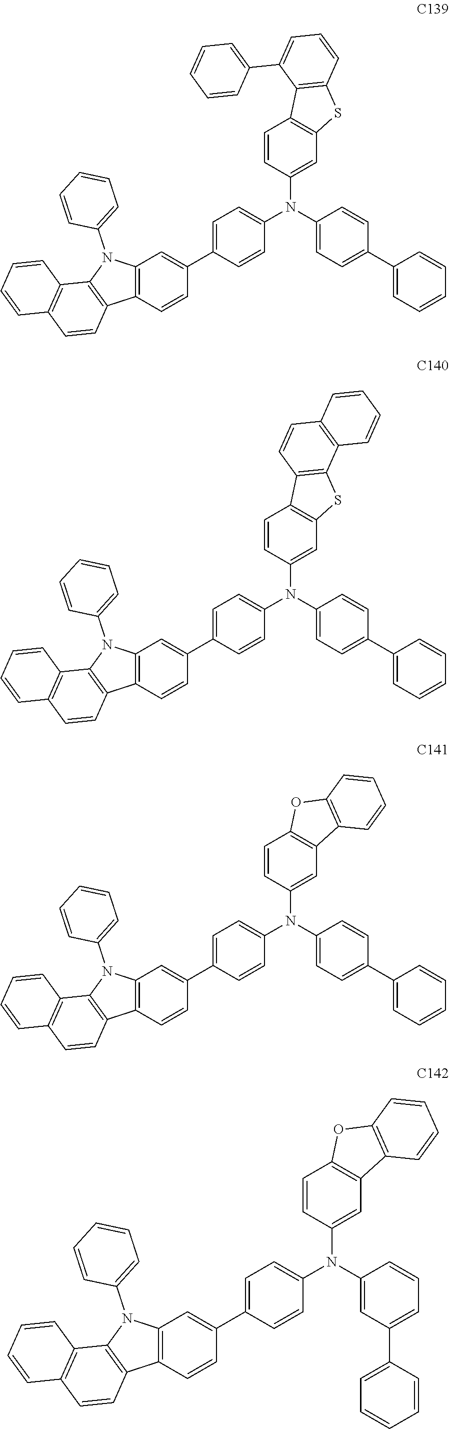 [SMILES: c1ccc(-c2ccc(N(c3ccc(-c4ccc5c6ccc7ccccc7c6n(-c6ccccc6)c5c4)cc3)c3ccc4c(c3)sc3cccc(-c5ccccc5)c34)cc2)cc1 ; c1ccc(-c2ccc(N(c3ccc(-c4ccc5c6ccc7ccccc7c6n(-c6ccccc6)c5c4)cc3)c3ccc4c(c3)sc3c5ccccc5ccc43)cc2)cc1 ; c1ccc(-c2ccc(N(c3ccc(-c4ccc5c6ccc7ccccc7c6n(-c6ccccc6)c5c4)cc3)c3ccc4oc5ccccc5c4c3)cc2)cc1 ; c1ccc(-c2cccc(N(c3ccc(-c4ccc5c6ccc7ccccc7c6n(-c6ccccc6)c5c4)cc3)c3ccc4oc5ccccc5c4c3)c2)cc1] 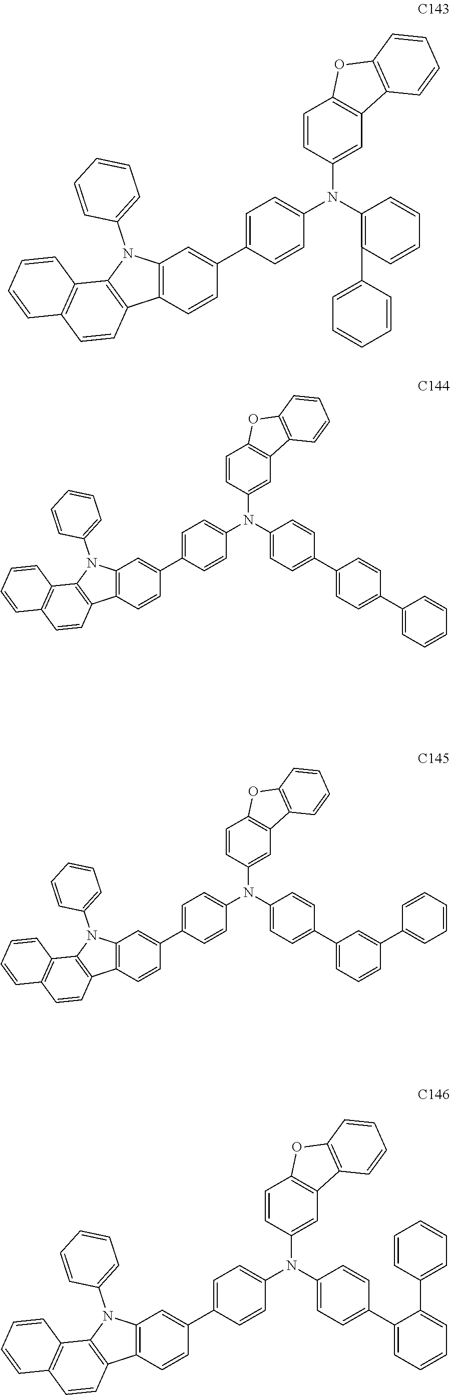 [SMILES: c1ccc(-c2ccccc2N(c2ccc(-c3ccc4c5ccc6ccccc6c5n(-c5ccccc5)c4c3)cc2)c2ccc3oc4ccccc4c3c2)cc1 ; c1ccc(-c2ccc(-c3ccc(N(c4ccc(-c5ccc6c7ccc8ccccc8c7n(-c7ccccc7)c6c5)cc4)c4ccc5oc6ccccc6c5c4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5oc6ccccc6c5c4)cc3)c2)cc1 ; c1ccc(-c2ccccc2-c2ccc(N(c3ccc(-c4ccc5c6ccc7ccccc7c6n(-c6ccccc6)c5c4)cc3)c3ccc4oc5ccccc5c4c3)cc2)cc1] 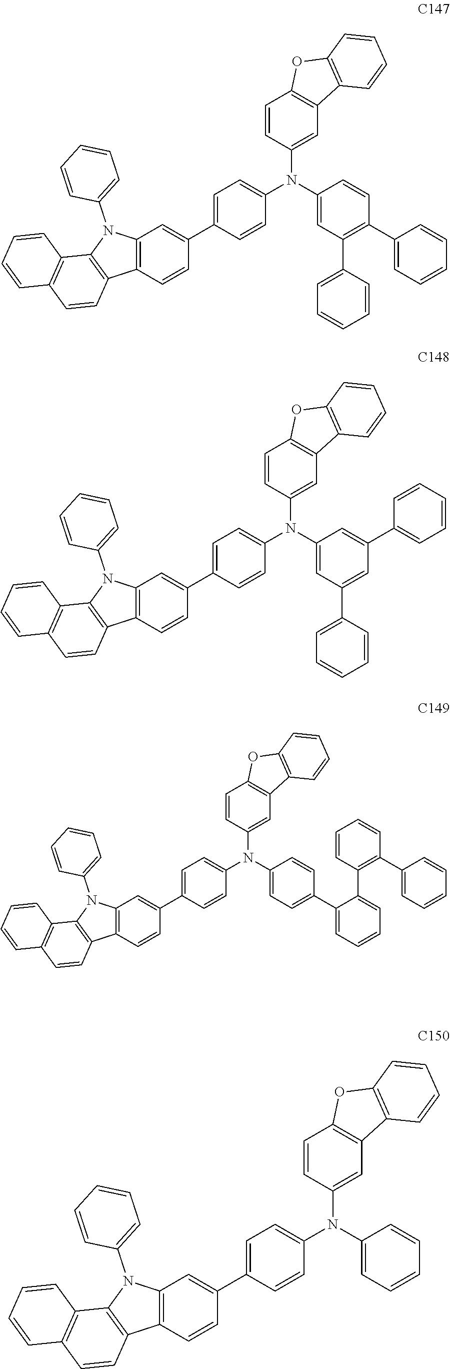 [SMILES: c1ccc(-c2ccc(N(c3ccc(-c4ccc5c6ccc7ccccc7c6n(-c6ccccc6)c5c4)cc3)c3ccc4oc5ccccc5c4c3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4oc5ccccc5c4c3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4oc5ccccc5c4c3)cc2)cc1 ; c1ccc(N(c2ccc(-c3ccc4c5ccc6ccccc6c5n(-c5ccccc5)c4c3)cc2)c2ccc3oc4ccccc4c3c2)cc1] 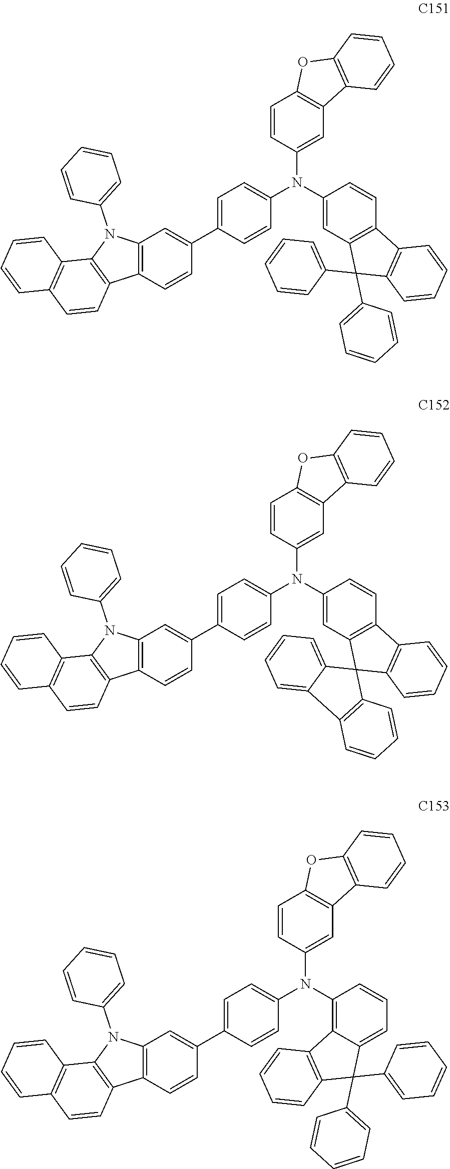 [SMILES: c1ccc(-n2c3cc(-c4ccc(N(c5ccc6c(c5)C(c5ccccc5)(c5ccccc5)c5ccccc5-6)c5ccc6oc7ccccc7c6c5)cc4)ccc3c3ccc4ccccc4c32)cc1 ; c1ccc(-n2c3cc(-c4ccc(N(c5ccc6c(c5)C5(c7ccccc7-c7ccccc75)c5ccccc5-6)c5ccc6oc7ccccc7c6c5)cc4)ccc3c3ccc4ccccc4c32)cc1 ; c1ccc(-n2c3cc(-c4ccc(N(c5ccc6oc7ccccc7c6c5)c5cccc6c5-c5ccccc5C6(c5ccccc5)c5ccccc5)cc4)ccc3c3ccc4ccccc4c32)cc1] 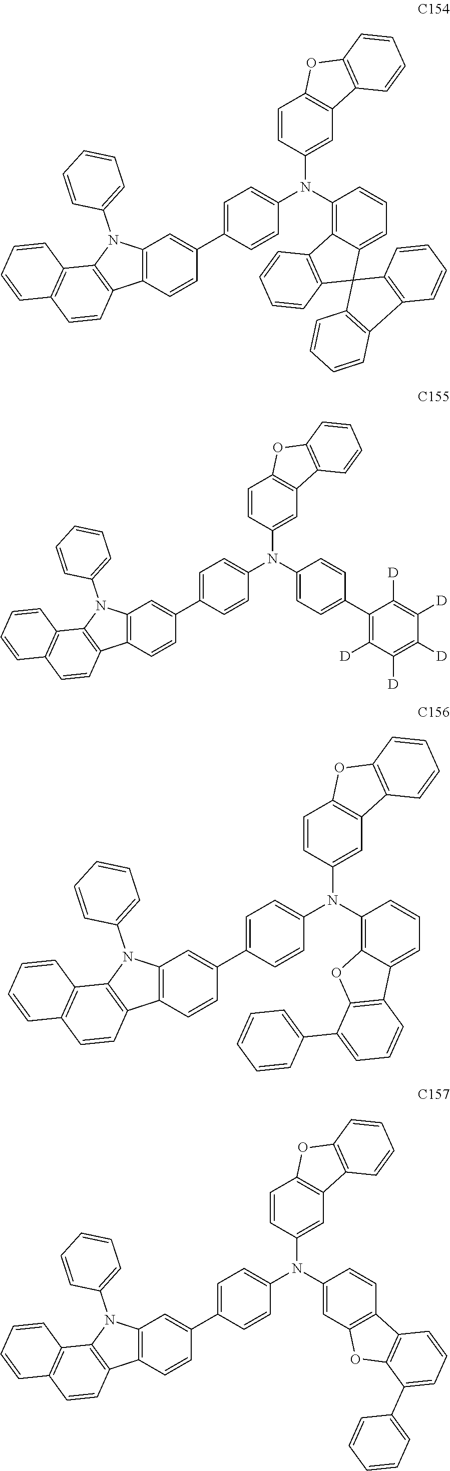 [SMILES: c1ccc(-n2c3cc(-c4ccc(N(c5ccc6oc7ccccc7c6c5)c5cccc6c5-c5ccccc5C65c6ccccc6-c6ccccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4oc5ccccc5c4c3)cc2)c([2H])c1[2H] ; c1ccc(-c2cccc3c2oc2c(N(c4ccc(-c5ccc6c7ccc8ccccc8c7n(-c7ccccc7)c6c5)cc4)c4ccc5oc6ccccc6c5c4)cccc23)cc1 ; c1ccc(-c2cccc3c2oc2cc(N(c4ccc(-c5ccc6c7ccc8ccccc8c7n(-c7ccccc7)c6c5)cc4)c4ccc5oc6ccccc6c5c4)ccc23)cc1] 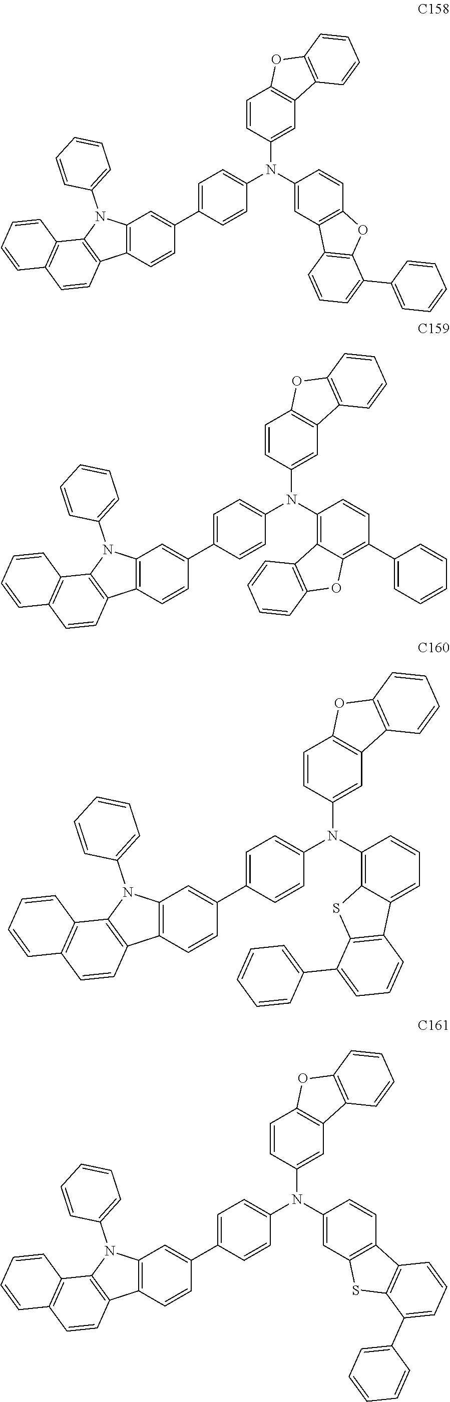 [SMILES: c1ccc(-c2cccc3c2oc2ccc(N(c4ccc(-c5ccc6c7ccc8ccccc8c7n(-c7ccccc7)c6c5)cc4)c4ccc5oc6ccccc6c5c4)cc23)cc1 ; c1ccc(-c2ccc(N(c3ccc(-c4ccc5c6ccc7ccccc7c6n(-c6ccccc6)c5c4)cc3)c3ccc4oc5ccccc5c4c3)c3c2oc2ccccc23)cc1 ; c1ccc(-c2cccc3c2sc2c(N(c4ccc(-c5ccc6c7ccc8ccccc8c7n(-c7ccccc7)c6c5)cc4)c4ccc5oc6ccccc6c5c4)cccc23)cc1 ; c1ccc(-c2cccc3c2sc2cc(N(c4ccc(-c5ccc6c7ccc8ccccc8c7n(-c7ccccc7)c6c5)cc4)c4ccc5oc6ccccc6c5c4)ccc23)cc1] 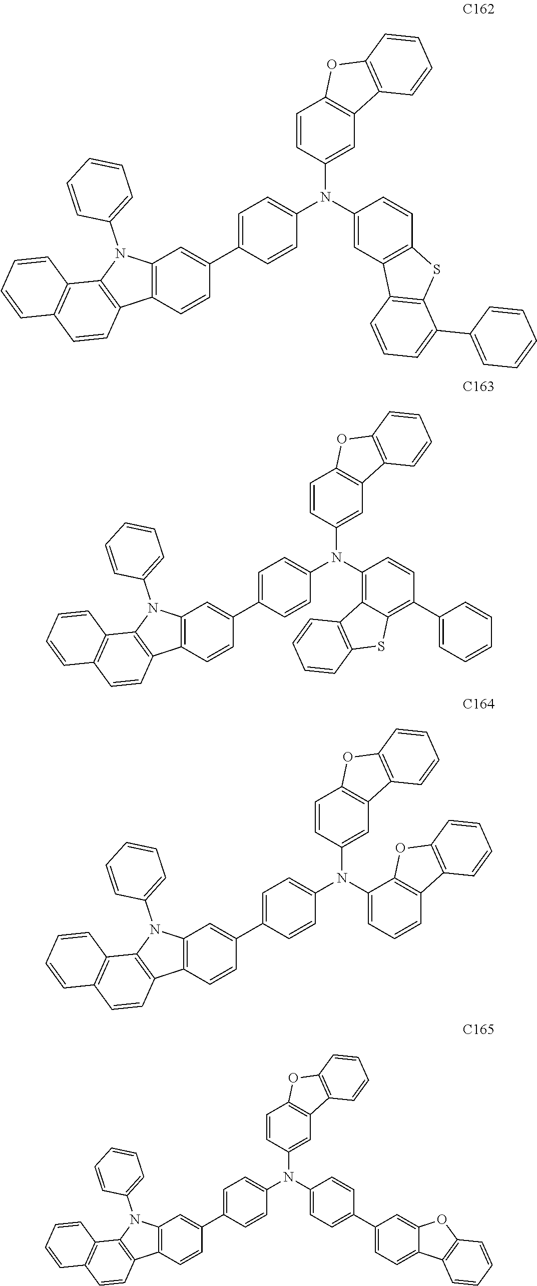 [SMILES: c1ccc(-c2cccc3c2sc2ccc(N(c4ccc(-c5ccc6c7ccc8ccccc8c7n(-c7ccccc7)c6c5)cc4)c4ccc5oc6ccccc6c5c4)cc23)cc1 ; c1ccc(-c2ccc(N(c3ccc(-c4ccc5c6ccc7ccccc7c6n(-c6ccccc6)c5c4)cc3)c3ccc4oc5ccccc5c4c3)c3c2sc2ccccc23)cc1 ; c1ccc(-n2c3cc(-c4ccc(N(c5ccc6oc7ccccc7c6c5)c5cccc6c5oc5ccccc56)cc4)ccc3c3ccc4ccccc4c32)cc1 ; c1ccc(-n2c3cc(-c4ccc(N(c5ccc(-c6ccc7c(c6)oc6ccccc67)cc5)c5ccc6oc7ccccc7c6c5)cc4)ccc3c3ccc4ccccc4c32)cc1] 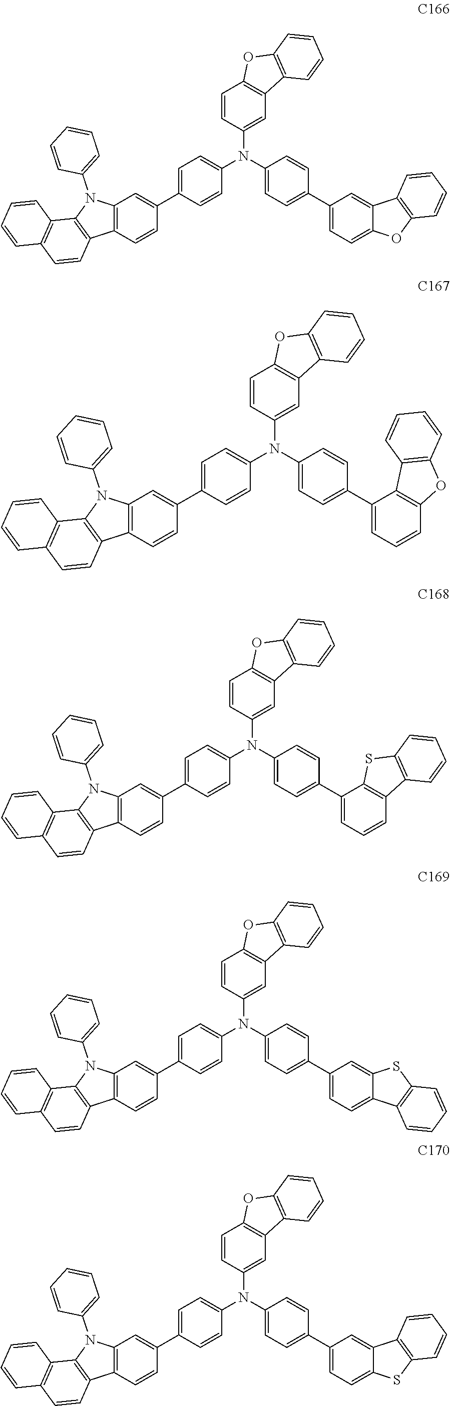 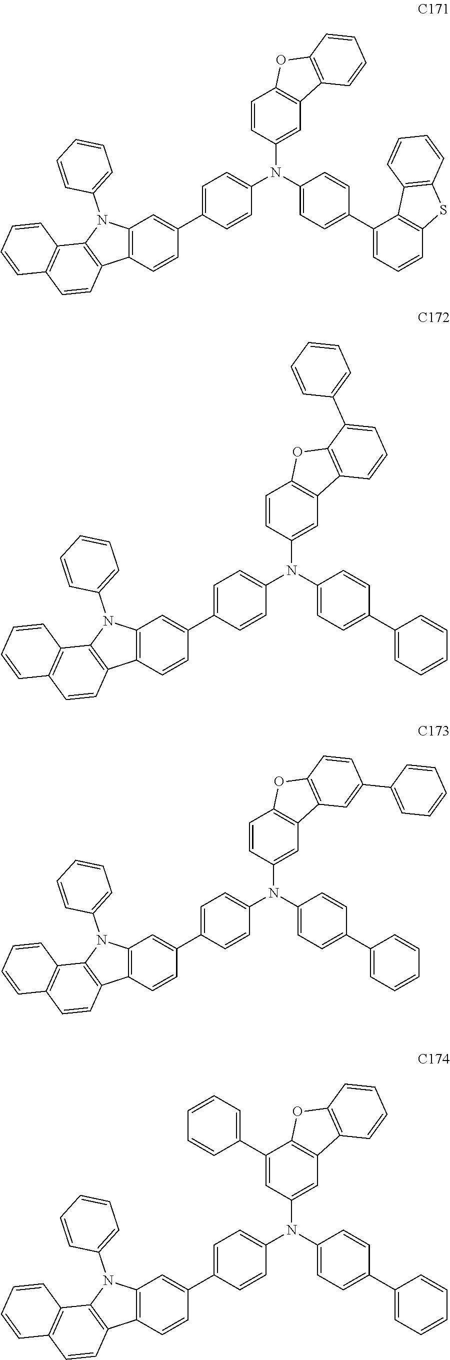 [SMILES: c1ccc(-n2c3cc(-c4ccc(N(c5ccc(-c6cccc7sc8ccccc8c67)cc5)c5ccc6oc7ccccc7c6c5)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4oc5c(-c6ccccc6)cccc5c4c3)cc2)cc1 ; c1ccc(-c2ccc(N(c3ccc(-c4ccc5c6ccc7ccccc7c6n(-c6ccccc6)c5c4)cc3)c3ccc4oc5ccc(-c6ccccc6)cc5c4c3)cc2)cc1 ; c1ccc(-c2ccc(N(c3ccc(-c4ccc5c6ccc7ccccc7c6n(-c6ccccc6)c5c4)cc3)c3cc(-c4ccccc4)c4oc5ccccc5c4c3)cc2)cc1] 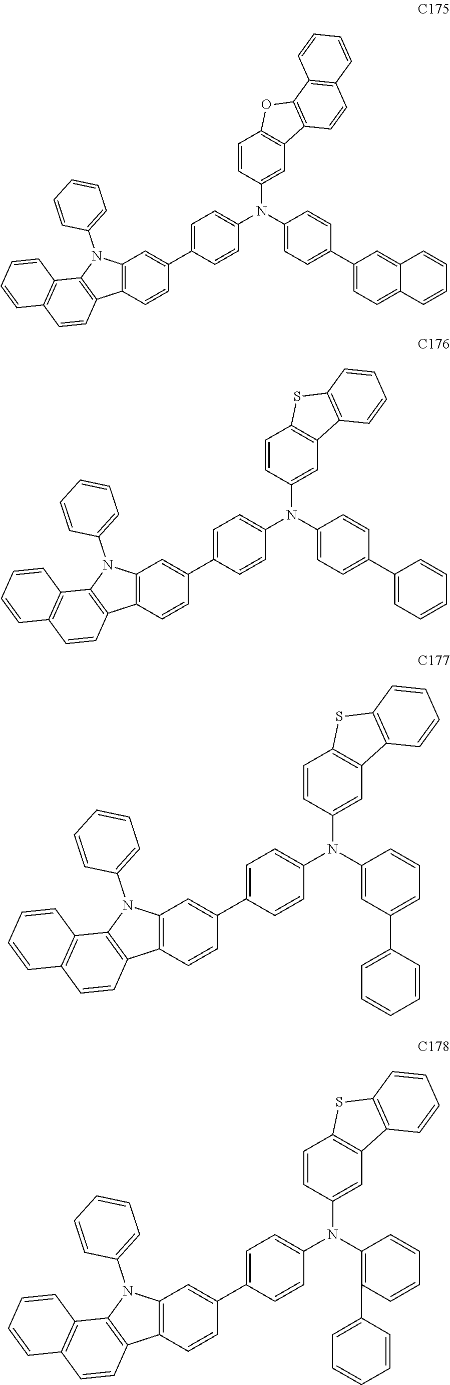 [SMILES: c1ccc(-n2c3cc(-c4ccc(N(c5ccc(-c6ccc7ccccc7c6)cc5)c5ccc6oc7c8ccccc8ccc7c6c5)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4sc5ccccc5c4c3)cc2)cc1 ; c1ccc(-c2cccc(N(c3ccc(-c4ccc5c6ccc7ccccc7c6n(-c6ccccc6)c5c4)cc3)c3ccc4sc5ccccc5c4c3)c2)cc1 ; c1ccc(-c2ccccc2N(c2ccc(-c3ccc4c5ccc6ccccc6c5n(-c5ccccc5)c4c3)cc2)c2ccc3sc4ccccc4c3c2)cc1] 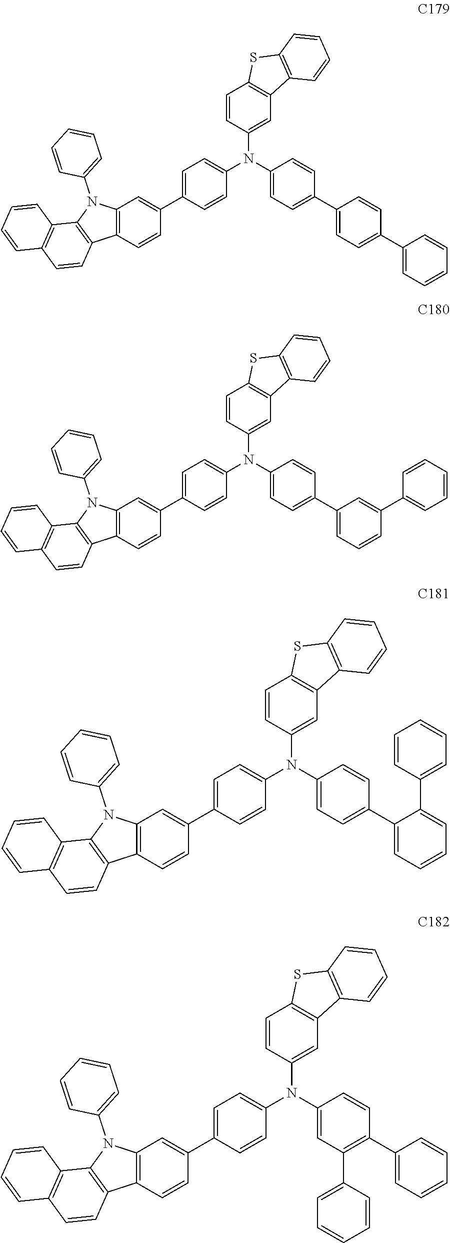 [SMILES: c1ccc(-c2ccc(-c3ccc(N(c4ccc(-c5ccc6c7ccc8ccccc8c7n(-c7ccccc7)c6c5)cc4)c4ccc5sc6ccccc6c5c4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5sc6ccccc6c5c4)cc3)c2)cc1 ; c1ccc(-c2ccccc2-c2ccc(N(c3ccc(-c4ccc5c6ccc7ccccc7c6n(-c6ccccc6)c5c4)cc3)c3ccc4sc5ccccc5c4c3)cc2)cc1 ; c1ccc(-c2ccc(N(c3ccc(-c4ccc5c6ccc7ccccc7c6n(-c6ccccc6)c5c4)cc3)c3ccc4sc5ccccc5c4c3)cc2-c2ccccc2)cc1] 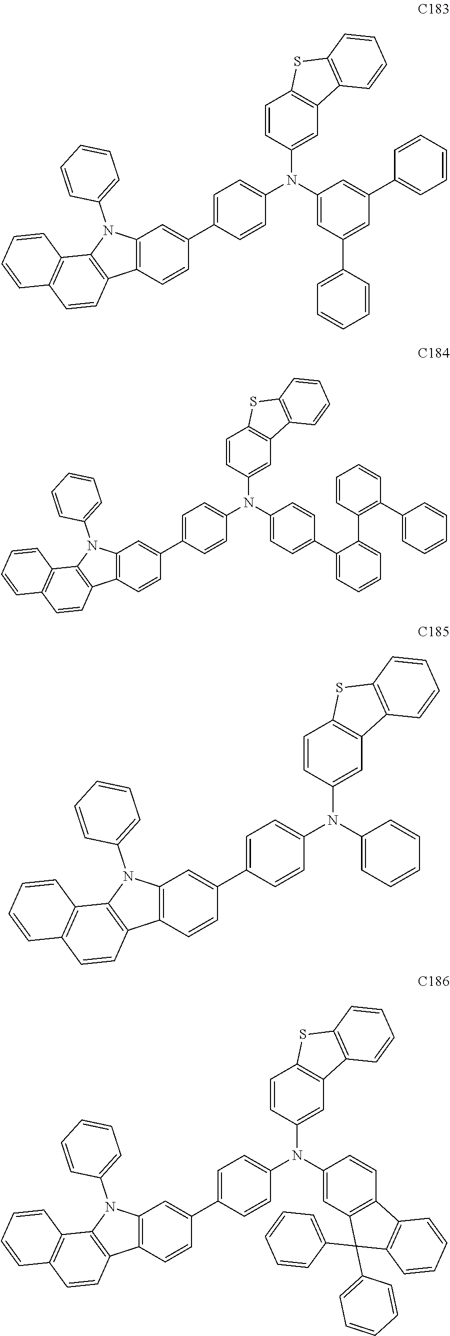 [SMILES: c1ccc(-c2cc(-c3ccccc3)cc(N(c3ccc(-c4ccc5c6ccc7ccccc7c6n(-c6ccccc6)c5c4)cc3)c3ccc4sc5ccccc5c4c3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4sc5ccccc5c4c3)cc2)cc1 ; c1ccc(N(c2ccc(-c3ccc4c5ccc6ccccc6c5n(-c5ccccc5)c4c3)cc2)c2ccc3sc4ccccc4c3c2)cc1 ; c1ccc(-n2c3cc(-c4ccc(N(c5ccc6c(c5)C(c5ccccc5)(c5ccccc5)c5ccccc5-6)c5ccc6sc7ccccc7c6c5)cc4)ccc3c3ccc4ccccc4c32)cc1] 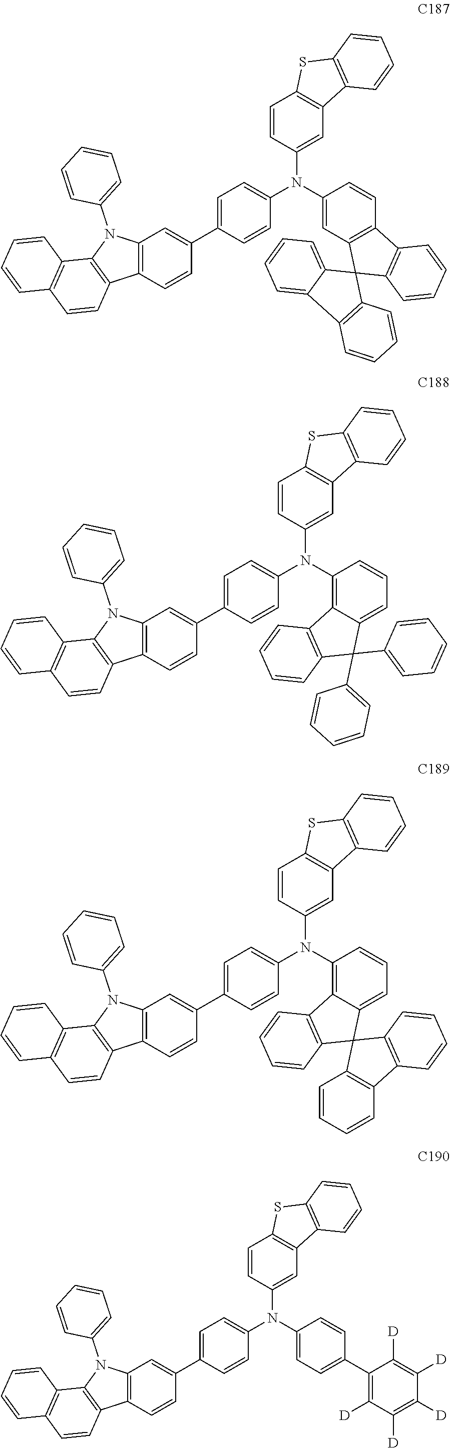 [SMILES: c1ccc(-n2c3cc(-c4ccc(N(c5ccc6c(c5)C5(c7ccccc7-c7ccccc75)c5ccccc5-6)c5ccc6sc7ccccc7c6c5)cc4)ccc3c3ccc4ccccc4c32)cc1 ; c1ccc(-n2c3cc(-c4ccc(N(c5ccc6sc7ccccc7c6c5)c5cccc6c5-c5ccccc5C6(c5ccccc5)c5ccccc5)cc4)ccc3c3ccc4ccccc4c32)cc1 ; c1ccc(-n2c3cc(-c4ccc(N(c5ccc6sc7ccccc7c6c5)c5cccc6c5-c5ccccc5C65c6ccccc6-c6ccccc65)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3ccc4sc5ccccc5c4c3)cc2)c([2H])c1[2H]] 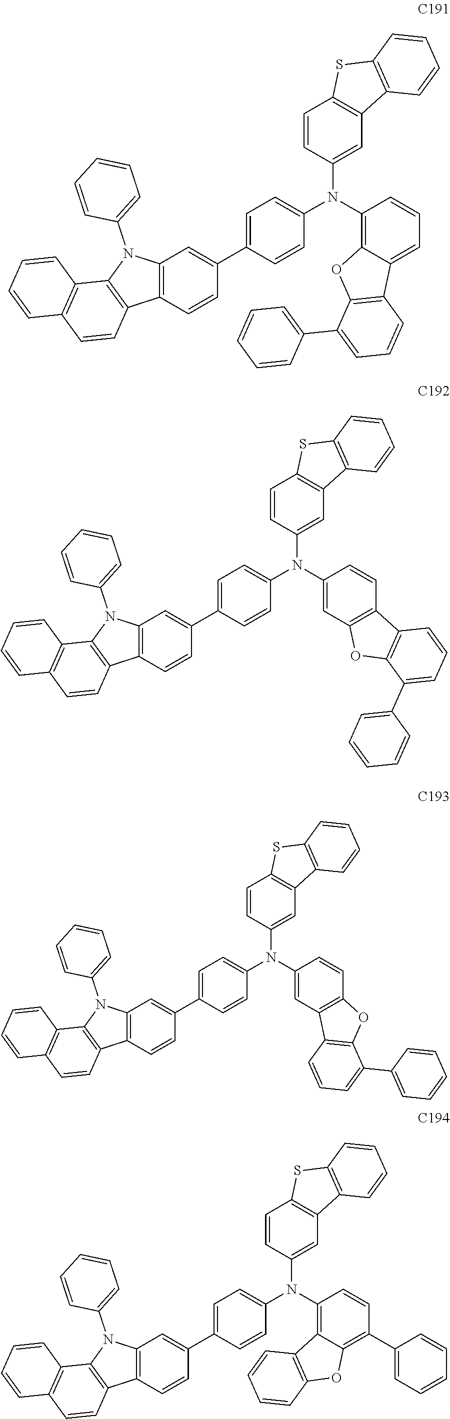 [SMILES: c1ccc(-c2cccc3c2oc2c(N(c4ccc(-c5ccc6c7ccc8ccccc8c7n(-c7ccccc7)c6c5)cc4)c4ccc5sc6ccccc6c5c4)cccc23)cc1 ; c1ccc(-c2cccc3c2oc2cc(N(c4ccc(-c5ccc6c7ccc8ccccc8c7n(-c7ccccc7)c6c5)cc4)c4ccc5sc6ccccc6c5c4)ccc23)cc1 ; c1ccc(-c2cccc3c2oc2ccc(N(c4ccc(-c5ccc6c7ccc8ccccc8c7n(-c7ccccc7)c6c5)cc4)c4ccc5sc6ccccc6c5c4)cc23)cc1 ; c1ccc(-c2ccc(N(c3ccc(-c4ccc5c6ccc7ccccc7c6n(-c6ccccc6)c5c4)cc3)c3ccc4sc5ccccc5c4c3)c3c2oc2ccccc23)cc1] 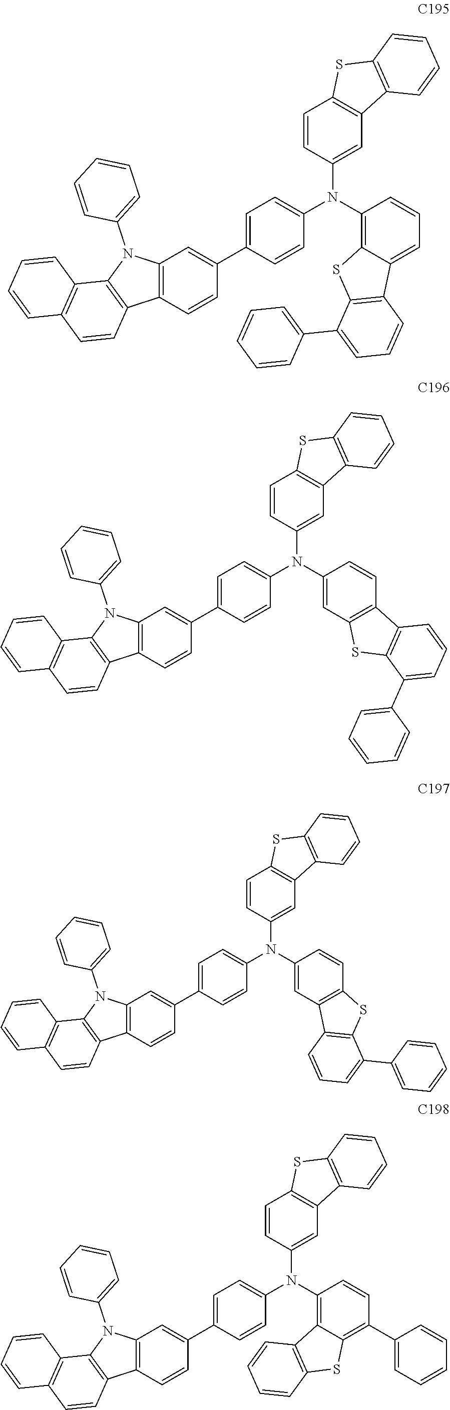 [SMILES: c1ccc(-c2cccc3c2sc2c(N(c4ccc(-c5ccc6c7ccc8ccccc8c7n(-c7ccccc7)c6c5)cc4)c4ccc5sc6ccccc6c5c4)cccc23)cc1 ; c1ccc(-c2cccc3c2sc2cc(N(c4ccc(-c5ccc6c7ccc8ccccc8c7n(-c7ccccc7)c6c5)cc4)c4ccc5sc6ccccc6c5c4)ccc23)cc1 ; c1ccc(-c2cccc3c2sc2ccc(N(c4ccc(-c5ccc6c7ccc8ccccc8c7n(-c7ccccc7)c6c5)cc4)c4ccc5sc6ccccc6c5c4)cc23)cc1 ; c1ccc(-c2ccc(N(c3ccc(-c4ccc5c6ccc7ccccc7c6n(-c6ccccc6)c5c4)cc3)c3ccc4sc5ccccc5c4c3)c3c2sc2ccccc23)cc1] 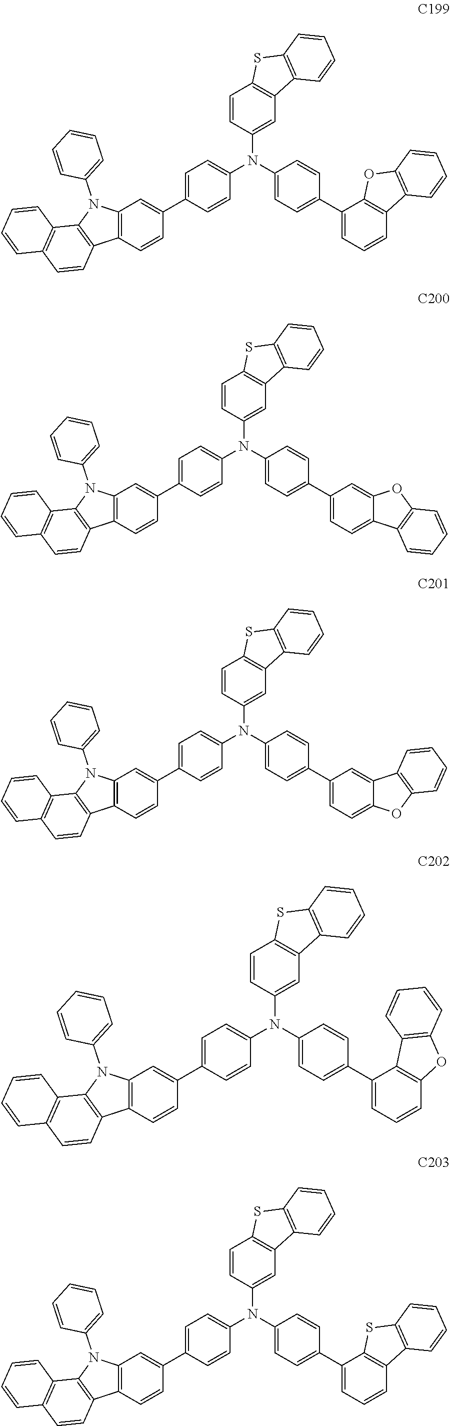 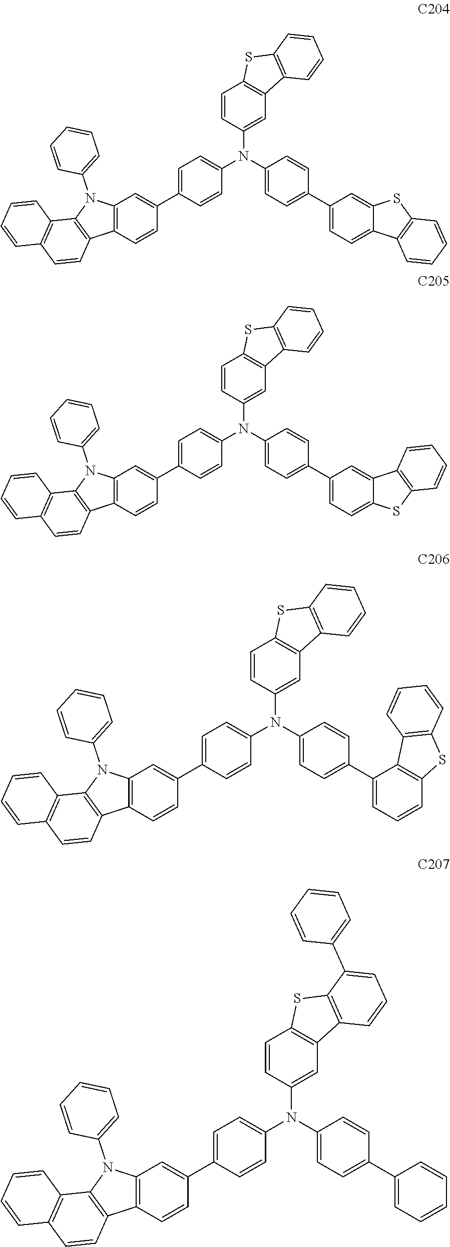 [SMILES: c1ccc(-n2c3cc(-c4ccc(N(c5ccc(-c6ccc7c(c6)sc6ccccc67)cc5)c5ccc6sc7ccccc7c6c5)cc4)ccc3c3ccc4ccccc4c32)cc1 ; c1ccc(-n2c3cc(-c4ccc(N(c5ccc(-c6ccc7sc8ccccc8c7c6)cc5)c5ccc6sc7ccccc7c6c5)cc4)ccc3c3ccc4ccccc4c32)cc1 ; c1ccc(-n2c3cc(-c4ccc(N(c5ccc(-c6cccc7sc8ccccc8c67)cc5)c5ccc6sc7ccccc7c6c5)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc4sc5c(-c6ccccc6)cccc5c4c3)cc2)cc1] 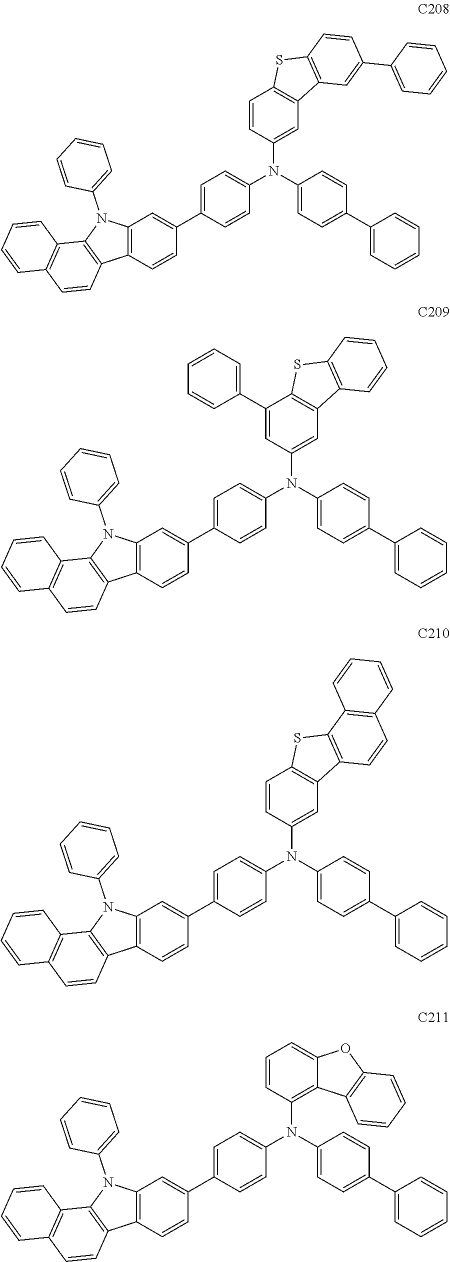 [SMILES: c1ccc(-c2ccc(N(c3ccc(-c4ccc5c6ccc7ccccc7c6n(-c6ccccc6)c5c4)cc3)c3ccc4sc5ccc(-c6ccccc6)cc5c4c3)cc2)cc1 ; c1ccc(-c2ccc(N(c3ccc(-c4ccc5c6ccc7ccccc7c6n(-c6ccccc6)c5c4)cc3)c3cc(-c4ccccc4)c4sc5ccccc5c4c3)cc2)cc1 ; c1ccc(-c2ccc(N(c3ccc(-c4ccc5c6ccc7ccccc7c6n(-c6ccccc6)c5c4)cc3)c3ccc4sc5c6ccccc6ccc5c4c3)cc2)cc1 ; c1ccc(-c2ccc(N(c3ccc(-c4ccc5c6ccc7ccccc7c6n(-c6ccccc6)c5c4)cc3)c3cccc4oc5ccccc5c34)cc2)cc1] 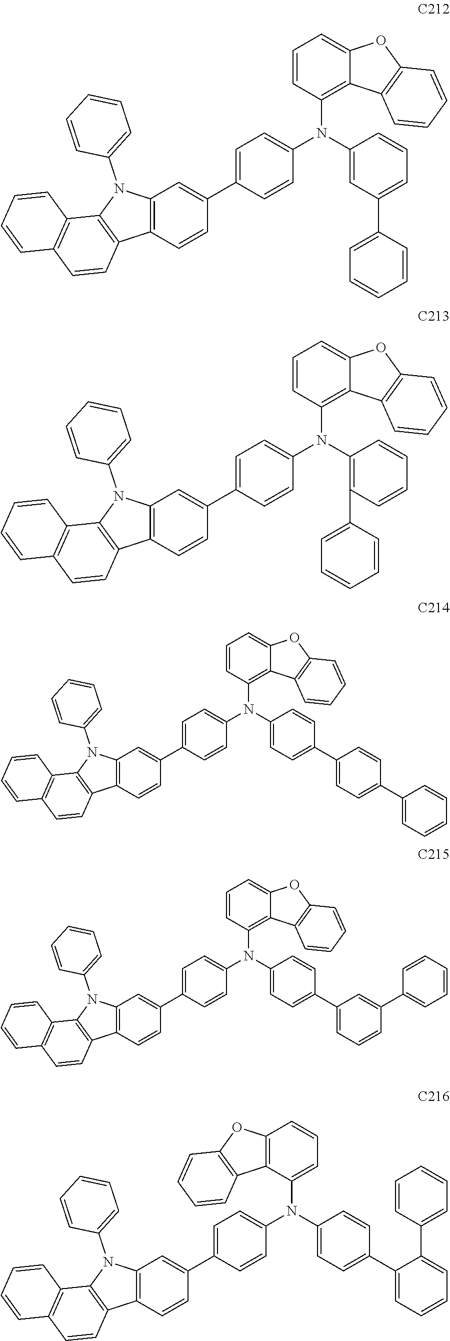 [SMILES: c1ccc(-c2cccc(N(c3ccc(-c4ccc5c6ccc7ccccc7c6n(-c6ccccc6)c5c4)cc3)c3cccc4oc5ccccc5c34)c2)cc1 ; c1ccc(-c2ccccc2N(c2ccc(-c3ccc4c5ccc6ccccc6c5n(-c5ccccc5)c4c3)cc2)c2cccc3oc4ccccc4c23)cc1 ; c1ccc(-c2ccc(-c3ccc(N(c4ccc(-c5ccc6c7ccc8ccccc8c7n(-c7ccccc7)c6c5)cc4)c4cccc5oc6ccccc6c45)cc3)cc2)cc1 ; c1ccc(-c2cccc(-c3ccc(N(c4ccc(-c5ccc6c7ccc8ccccc8c7n(-c7ccccc7)c6c5)cc4)c4cccc5oc6ccccc6c45)cc3)c2)cc1 ; c1ccc(-c2ccccc2-c2ccc(N(c3ccc(-c4ccc5c6ccc7ccccc7c6n(-c6ccccc6)c5c4)cc3)c3cccc4oc5ccccc5c34)cc2)cc1] 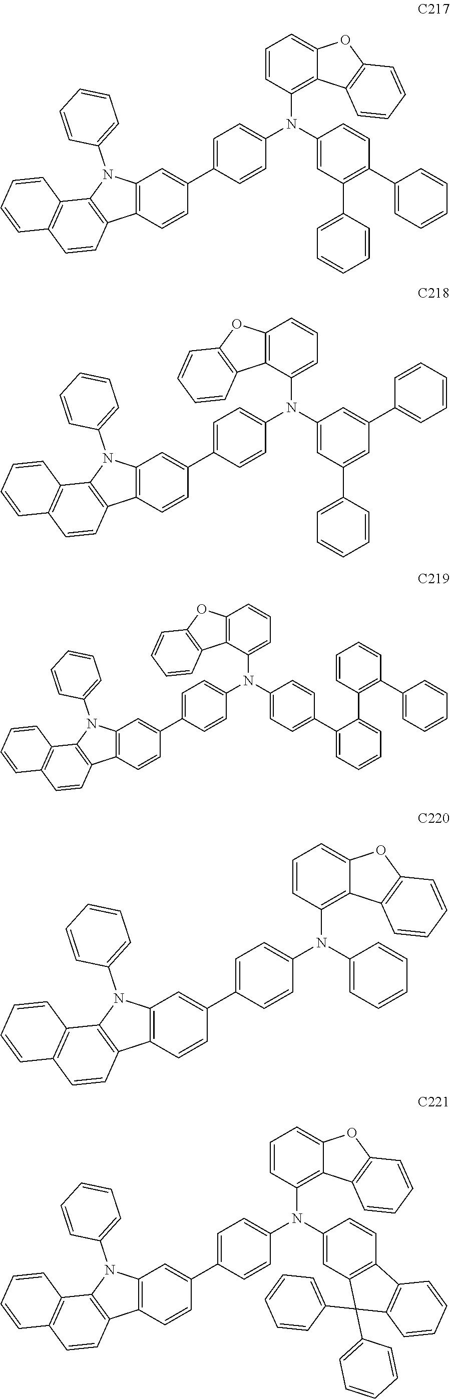 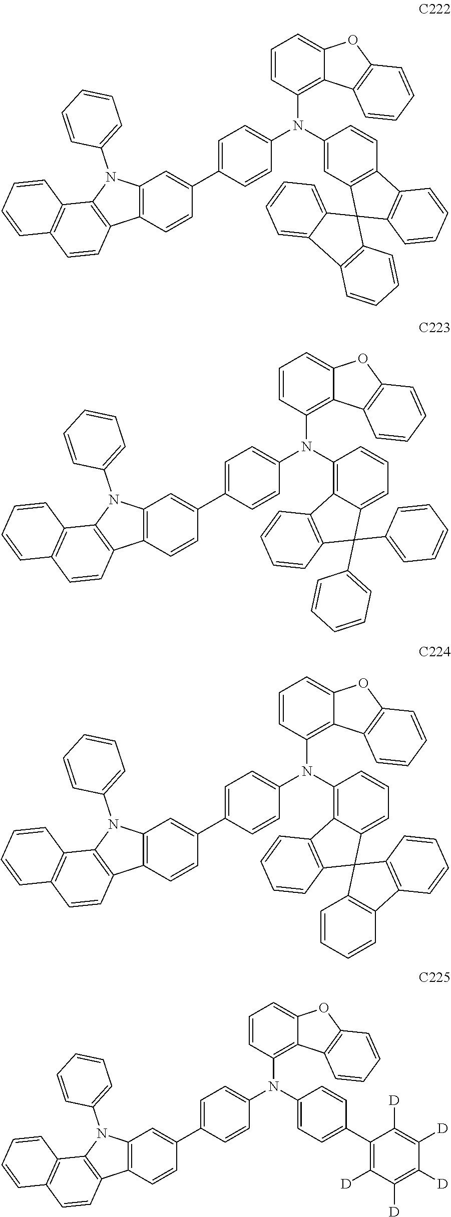 [SMILES: c1ccc(-n2c3cc(-c4ccc(N(c5ccc6c(c5)C5(c7ccccc7-c7ccccc75)c5ccccc5-6)c5cccc6oc7ccccc7c56)cc4)ccc3c3ccc4ccccc4c32)cc1 ; c1ccc(-n2c3cc(-c4ccc(N(c5cccc6c5-c5ccccc5C6(c5ccccc5)c5ccccc5)c5cccc6oc7ccccc7c56)cc4)ccc3c3ccc4ccccc4c32)cc1 ; c1ccc(-n2c3cc(-c4ccc(N(c5cccc6c5-c5ccccc5C65c6ccccc6-c6ccccc65)c5cccc6oc7ccccc7c56)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3cccc4oc5ccccc5c34)cc2)c([2H])c1[2H]] 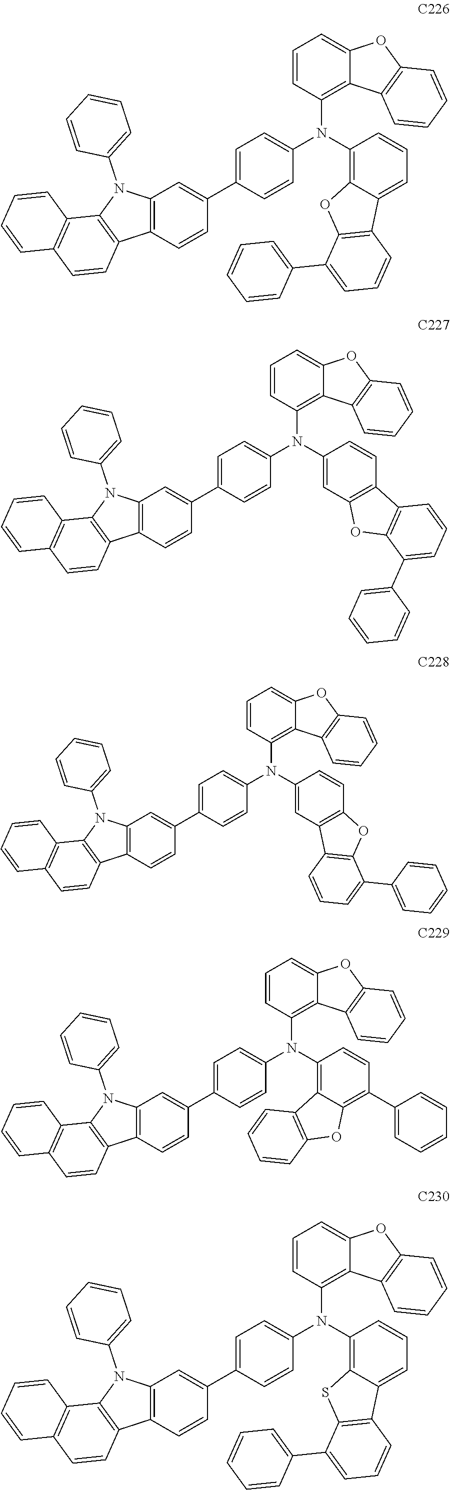 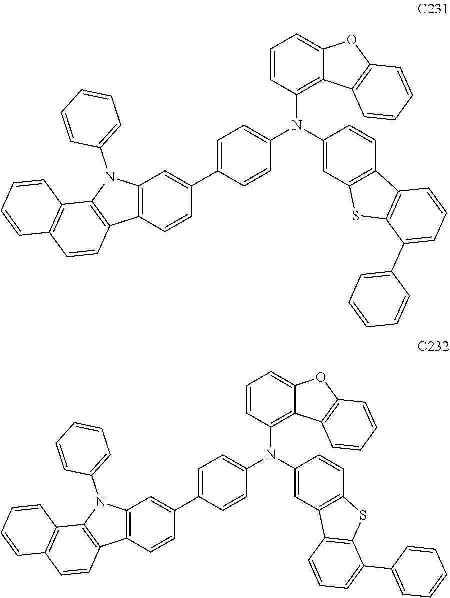 [SMILES: c1ccc(-c2cccc3c2sc2cc(N(c4ccc(-c5ccc6c7ccc8ccccc8c7n(-c7ccccc7)c6c5)cc4)c4cccc5oc6ccccc6c45)ccc23)cc1 ; c1ccc(-c2cccc3c2sc2ccc(N(c4ccc(-c5ccc6c7ccc8ccccc8c7n(-c7ccccc7)c6c5)cc4)c4cccc5oc6ccccc6c45)cc23)cc1] 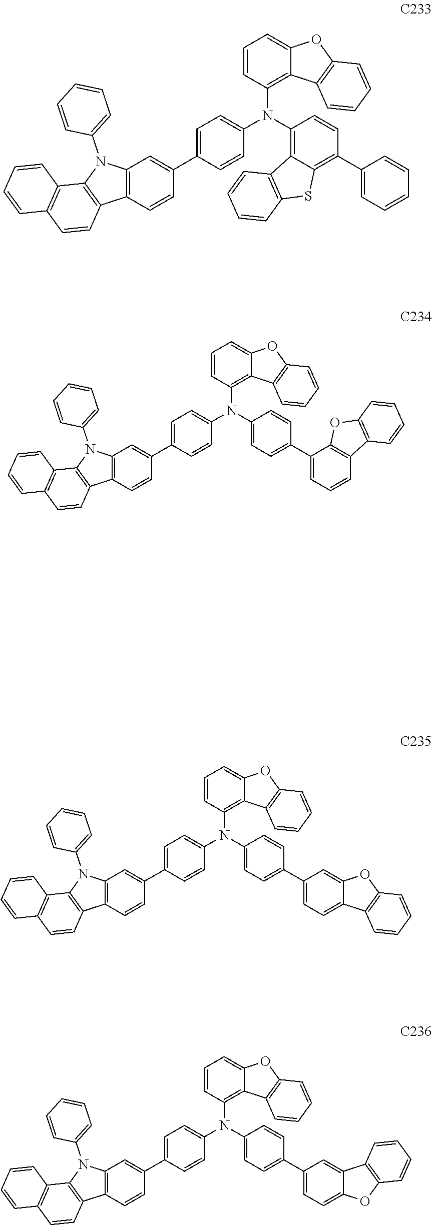 [SMILES: c1ccc(-c2ccc(N(c3ccc(-c4ccc5c6ccc7ccccc7c6n(-c6ccccc6)c5c4)cc3)c3cccc4oc5ccccc5c34)c3c2sc2ccccc23)cc1 ; c1ccc(-n2c3cc(-c4ccc(N(c5ccc(-c6cccc7c6oc6ccccc67)cc5)c5cccc6oc7ccccc7c56)cc4)ccc3c3ccc4ccccc4c32)cc1 ; c1ccc(-n2c3cc(-c4ccc(N(c5ccc(-c6ccc7c(c6)oc6ccccc67)cc5)c5cccc6oc7ccccc7c56)cc4)ccc3c3ccc4ccccc4c32)cc1 ; c1ccc(-n2c3cc(-c4ccc(N(c5ccc(-c6ccc7oc8ccccc8c7c6)cc5)c5cccc6oc7ccccc7c56)cc4)ccc3c3ccc4ccccc4c32)cc1] 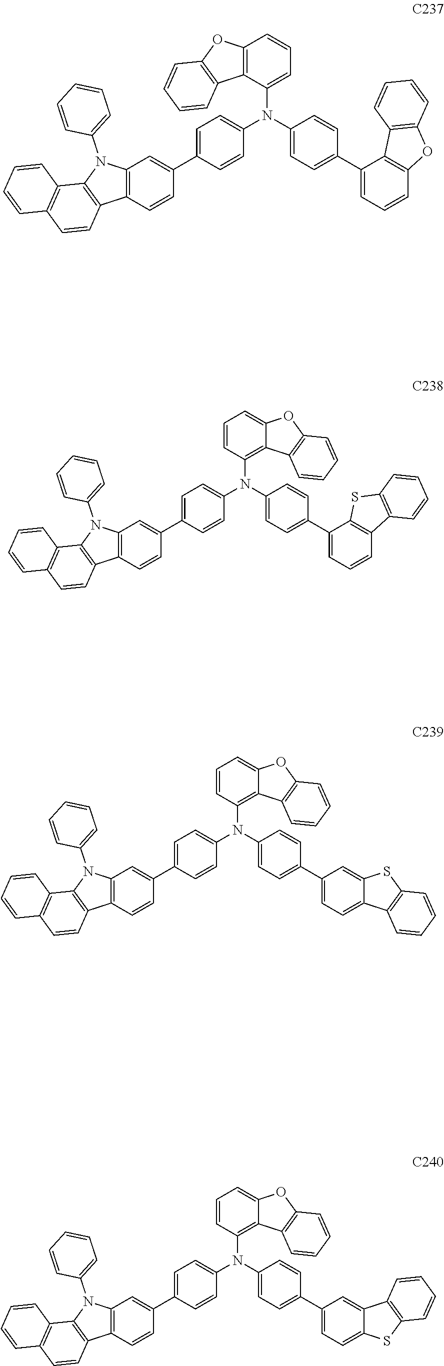 [SMILES: c1ccc(-n2c3cc(-c4ccc(N(c5ccc(-c6cccc7oc8ccccc8c67)cc5)c5cccc6oc7ccccc7c56)cc4)ccc3c3ccc4ccccc4c32)cc1 ; c1ccc(-n2c3cc(-c4ccc(N(c5ccc(-c6cccc7c6sc6ccccc67)cc5)c5cccc6oc7ccccc7c56)cc4)ccc3c3ccc4ccccc4c32)cc1 ; c1ccc(-n2c3cc(-c4ccc(N(c5ccc(-c6ccc7c(c6)sc6ccccc67)cc5)c5cccc6oc7ccccc7c56)cc4)ccc3c3ccc4ccccc4c32)cc1 ; c1ccc(-n2c3cc(-c4ccc(N(c5ccc(-c6ccc7sc8ccccc8c7c6)cc5)c5cccc6oc7ccccc7c56)cc4)ccc3c3ccc4ccccc4c32)cc1] 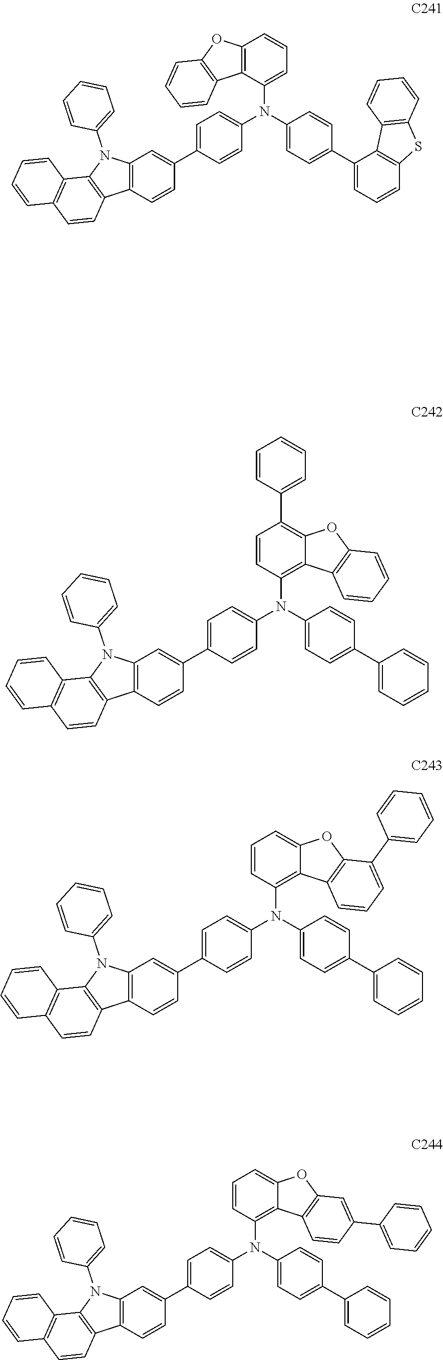 [SMILES: c1ccc(-n2c3cc(-c4ccc(N(c5ccc(-c6cccc7sc8ccccc8c67)cc5)c5cccc6oc7ccccc7c56)cc4)ccc3c3ccc4ccccc4c32)cc1 ; c1ccc(-c2ccc(N(c3ccc(-c4ccc5c6ccc7ccccc7c6n(-c6ccccc6)c5c4)cc3)c3ccc(-c4ccccc4)c4oc5ccccc5c34)cc2)cc1 ; c1ccc(-c2ccc(N(c3ccc(-c4ccc5c6ccc7ccccc7c6n(-c6ccccc6)c5c4)cc3)c3cccc4oc5c(-c6ccccc6)cccc5c34)cc2)cc1 ; c1ccc(-c2ccc(N(c3ccc(-c4ccc5c6ccc7ccccc7c6n(-c6ccccc6)c5c4)cc3)c3cccc4oc5cc(-c6ccccc6)ccc5c34)cc2)cc1] 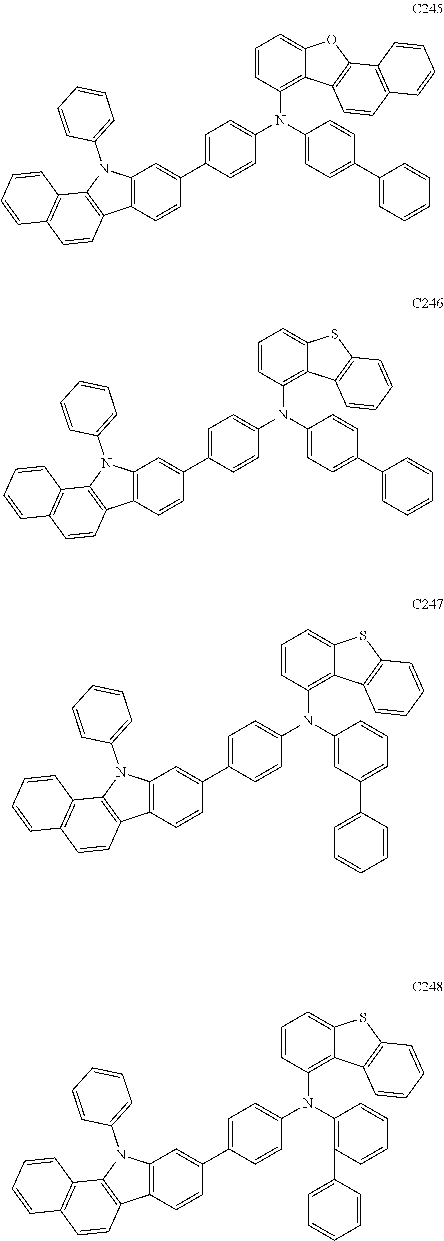 [SMILES: c1ccc(-c2ccc(N(c3ccc(-c4ccc5c6ccc7ccccc7c6n(-c6ccccc6)c5c4)cc3)c3cccc4oc5c6ccccc6ccc5c34)cc2)cc1 ; c1ccc(-c2ccc(N(c3ccc(-c4ccc5c6ccc7ccccc7c6n(-c6ccccc6)c5c4)cc3)c3cccc4sc5ccccc5c34)cc2)cc1 ; c1ccc(-c2cccc(N(c3ccc(-c4ccc5c6ccc7ccccc7c6n(-c6ccccc6)c5c4)cc3)c3cccc4sc5ccccc5c34)c2)cc1 ; c1ccc(-c2ccccc2N(c2ccc(-c3ccc4c5ccc6ccccc6c5n(-c5ccccc5)c4c3)cc2)c2cccc3sc4ccccc4c23)cc1] 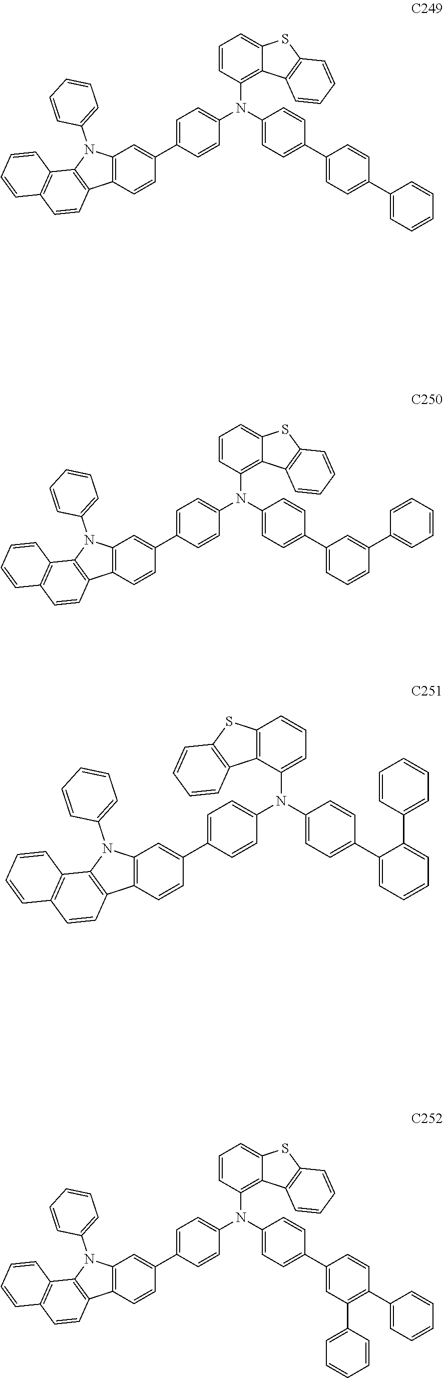 [SMILES: c1ccc(-c2ccc(-c3ccc(N(c4ccc(-c5ccc6c7ccc8ccccc8c7n(-c7ccccc7)c6c5)cc4)c4cccc5sc6ccccc6c45)cc3)cc2)cc1 ; c1ccc(-c2cccc(-c3ccc(N(c4ccc(-c5ccc6c7ccc8ccccc8c7n(-c7ccccc7)c6c5)cc4)c4cccc5sc6ccccc6c45)cc3)c2)cc1 ; c1ccc(-c2ccccc2-c2ccc(N(c3ccc(-c4ccc5c6ccc7ccccc7c6n(-c6ccccc6)c5c4)cc3)c3cccc4sc5ccccc5c34)cc2)cc1 ; c1ccc(-c2ccc(-c3ccc(N(c4ccc(-c5ccc6c7ccc8ccccc8c7n(-c7ccccc7)c6c5)cc4)c4cccc5sc6ccccc6c45)cc3)cc2-c2ccccc2)cc1] 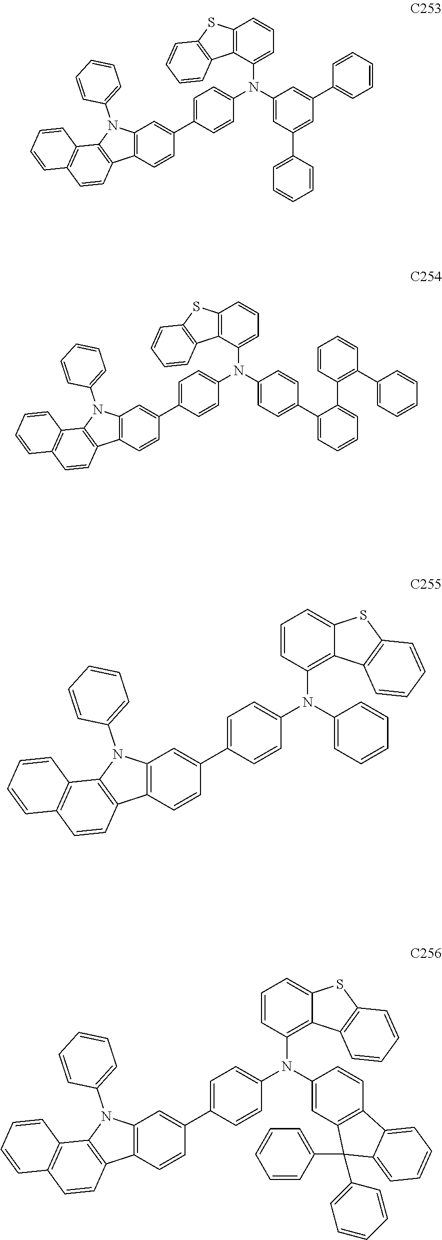 [SMILES: c1ccc(-c2cc(-c3ccccc3)cc(N(c3ccc(-c4ccc5c6ccc7ccccc7c6n(-c6ccccc6)c5c4)cc3)c3cccc4sc5ccccc5c34)c2)cc1 ; c1ccc(-c2ccccc2-c2ccccc2-c2ccc(N(c3ccc(-c4ccc5c6ccc7ccccc7c6n(-c6ccccc6)c5c4)cc3)c3cccc4sc5ccccc5c34)cc2)cc1 ; c1ccc(N(c2ccc(-c3ccc4c5ccc6ccccc6c5n(-c5ccccc5)c4c3)cc2)c2cccc3sc4ccccc4c23)cc1 ; c1ccc(-n2c3cc(-c4ccc(N(c5ccc6c(c5)C(c5ccccc5)(c5ccccc5)c5ccccc5-6)c5cccc6sc7ccccc7c56)cc4)ccc3c3ccc4ccccc4c32)cc1] 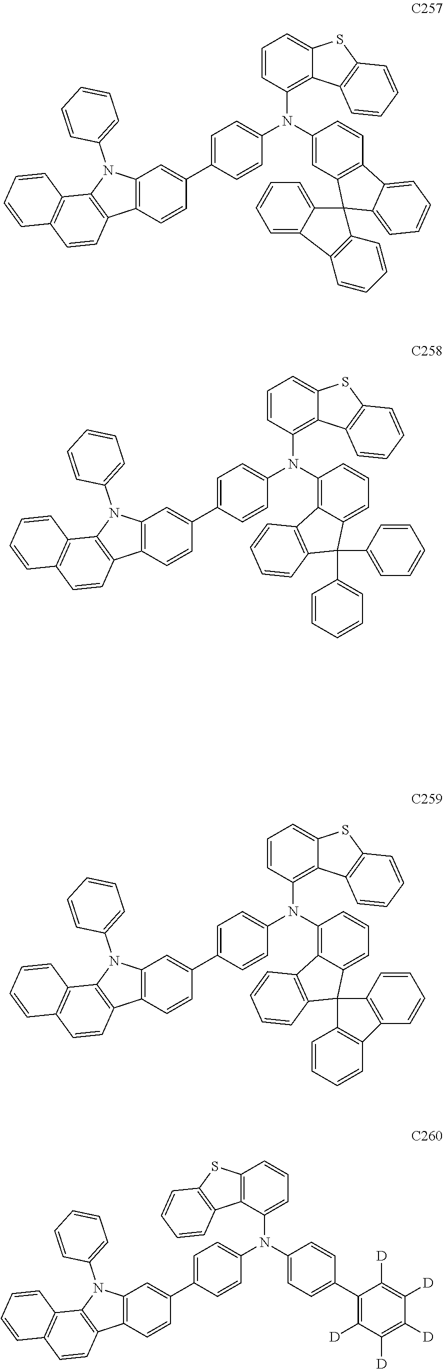 [SMILES: c1ccc(-n2c3cc(-c4ccc(N(c5ccc6c(c5)C5(c7ccccc7-c7ccccc75)c5ccccc5-6)c5cccc6sc7ccccc7c56)cc4)ccc3c3ccc4ccccc4c32)cc1 ; c1ccc(-n2c3cc(-c4ccc(N(c5cccc6c5-c5ccccc5C6(c5ccccc5)c5ccccc5)c5cccc6sc7ccccc7c56)cc4)ccc3c3ccc4ccccc4c32)cc1 ; c1ccc(-n2c3cc(-c4ccc(N(c5cccc6c5-c5ccccc5C65c6ccccc6-c6ccccc65)c5cccc6sc7ccccc7c56)cc4)ccc3c3ccc4ccccc4c32)cc1 ; [2H]c1c([2H])c([2H])c(-c2ccc(N(c3ccc(-c4ccc5c6ccc7ccccc7c6n(-c6ccccc6)c5c4)cc3)c3cccc4sc5ccccc5c34)cc2)c([2H])c1[2H]] 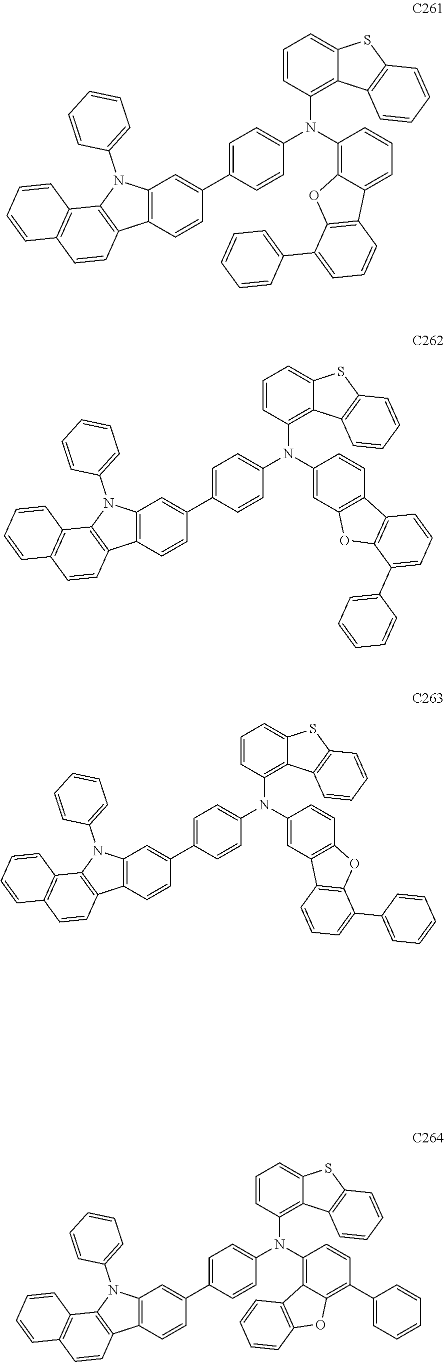 [SMILES: c1ccc(-c2cccc3c2oc2c(N(c4ccc(-c5ccc6c7ccc8ccccc8c7n(-c7ccccc7)c6c5)cc4)c4cccc5sc6ccccc6c45)cccc23)cc1 ; c1ccc(-c2cccc3c2oc2cc(N(c4ccc(-c5ccc6c7ccc8ccccc8c7n(-c7ccccc7)c6c5)cc4)c4cccc5sc6ccccc6c45)ccc23)cc1 ; c1ccc(-c2cccc3c2oc2ccc(N(c4ccc(-c5ccc6c7ccc8ccccc8c7n(-c7ccccc7)c6c5)cc4)c4cccc5sc6ccccc6c45)cc23)cc1 ; c1ccc(-c2ccc(N(c3ccc(-c4ccc5c6ccc7ccccc7c6n(-c6ccccc6)c5c4)cc3)c3cccc4sc5ccccc5c34)c3c2oc2ccccc23)cc1] 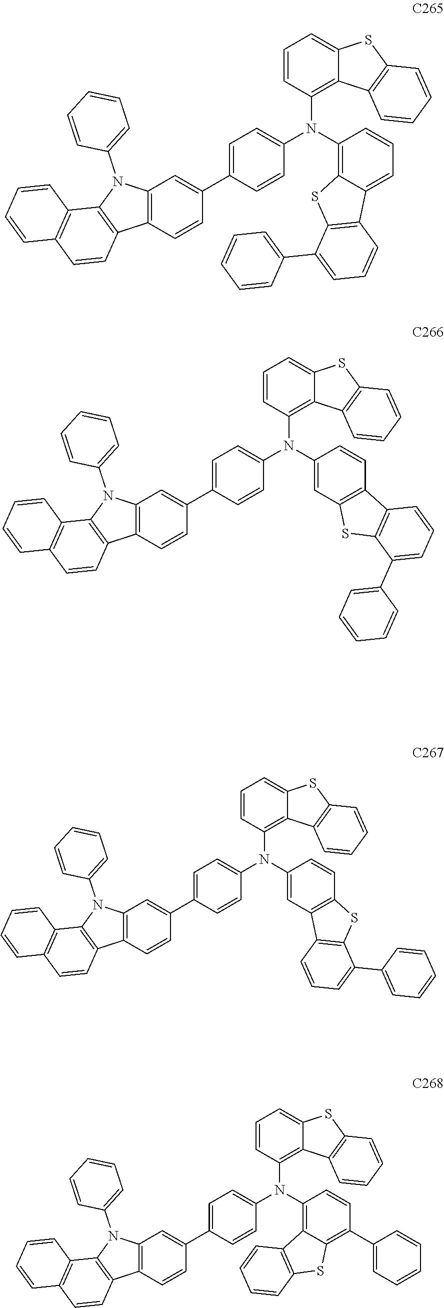 [SMILES: c1ccc(-c2cccc3c2sc2c(N(c4ccc(-c5ccc6c7ccc8ccccc8c7n(-c7ccccc7)c6c5)cc4)c4cccc5sc6ccccc6c45)cccc23)cc1 ; c1ccc(-c2cccc3c2sc2cc(N(c4ccc(-c5ccc6c7ccc8ccccc8c7n(-c7ccccc7)c6c5)cc4)c4cccc5sc6ccccc6c45)ccc23)cc1 ; c1ccc(-c2cccc3c2sc2ccc(N(c4ccc(-c5ccc6c7ccc8ccccc8c7n(-c7ccccc7)c6c5)cc4)c4cccc5sc6ccccc6c45)cc23)cc1 ; c1ccc(-c2ccc(N(c3ccc(-c4ccc5c6ccc7ccccc7c6n(-c6ccccc6)c5c4)cc3)c3cccc4sc5ccccc5c34)c3c2sc2ccccc23)cc1] 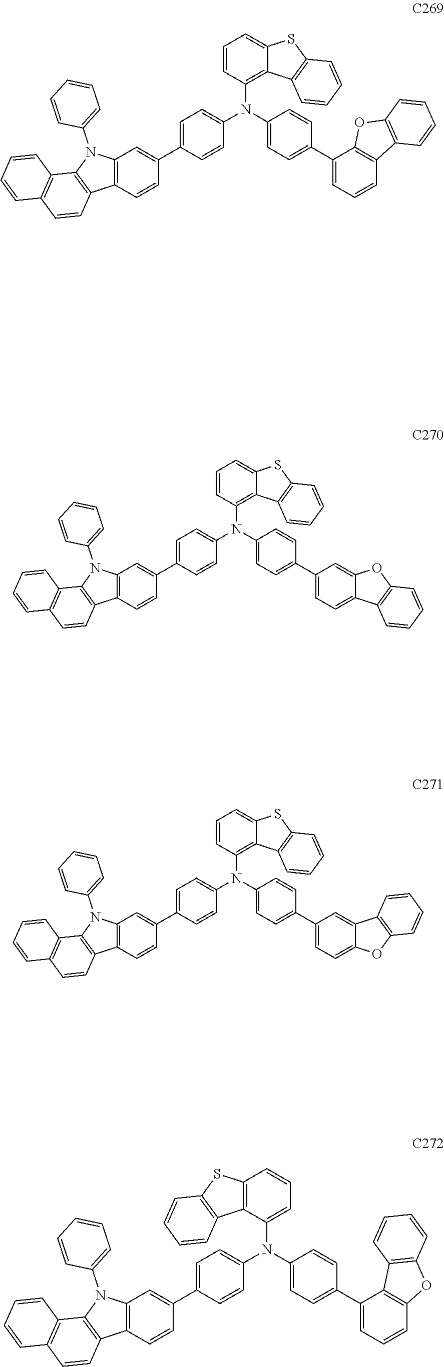 [SMILES: c1ccc(-n2c3cc(-c4ccc(N(c5ccc(-c6cccc7c6oc6ccccc67)cc5)c5cccc6sc7ccccc7c56)cc4)ccc3c3ccc4ccccc4c32)cc1 ; c1ccc(-n2c3cc(-c4ccc(N(c5ccc(-c6ccc7c(c6)oc6ccccc67)cc5)c5cccc6sc7ccccc7c56)cc4)ccc3c3ccc4ccccc4c32)cc1 ; c1ccc(-n2c3cc(-c4ccc(N(c5ccc(-c6ccc7oc8ccccc8c7c6)cc5)c5cccc6sc7ccccc7c56)cc4)ccc3c3ccc4ccccc4c32)cc1 ; c1ccc(-n2c3cc(-c4ccc(N(c5ccc(-c6cccc7oc8ccccc8c67)cc5)c5cccc6sc7ccccc7c56)cc4)ccc3c3ccc4ccccc4c32)cc1] 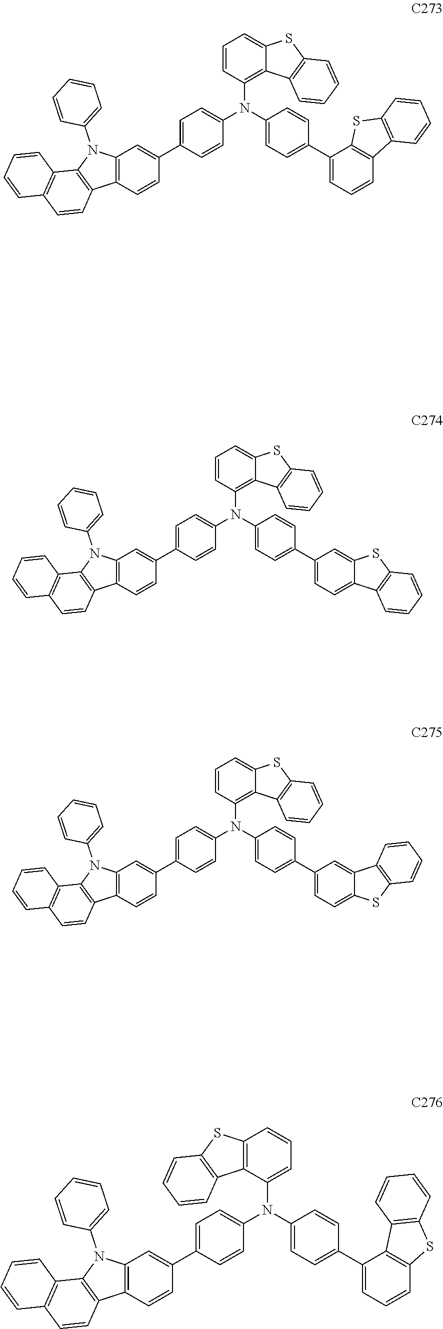 [SMILES: c1ccc(-n2c3cc(-c4ccc(N(c5ccc(-c6cccc7c6sc6ccccc67)cc5)c5cccc6sc7ccccc7c56)cc4)ccc3c3ccc4ccccc4c32)cc1 ; c1ccc(-n2c3cc(-c4ccc(N(c5ccc(-c6ccc7c(c6)sc6ccccc67)cc5)c5cccc6sc7ccccc7c56)cc4)ccc3c3ccc4ccccc4c32)cc1 ; c1ccc(-n2c3cc(-c4ccc(N(c5ccc(-c6ccc7sc8ccccc8c7c6)cc5)c5cccc6sc7ccccc7c56)cc4)ccc3c3ccc4ccccc4c32)cc1 ; c1ccc(-n2c3cc(-c4ccc(N(c5ccc(-c6cccc7sc8ccccc8c67)cc5)c5cccc6sc7ccccc7c56)cc4)ccc3c3ccc4ccccc4c32)cc1] 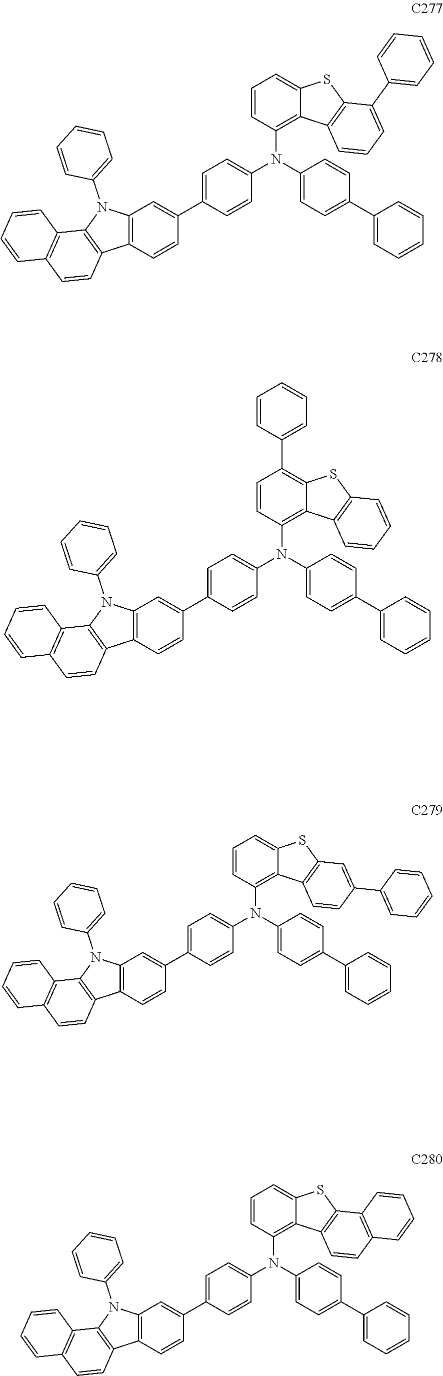 [SMILES: c1ccc(-c2ccc(N(c3ccc(-c4ccc5c6ccc7ccccc7c6n(-c6ccccc6)c5c4)cc3)c3cccc4sc5c(-c6ccccc6)cccc5c34)cc2)cc1 ; c1ccc(-c2ccc(N(c3ccc(-c4ccc5c6ccc7ccccc7c6n(-c6ccccc6)c5c4)cc3)c3ccc(-c4ccccc4)c4sc5ccccc5c34)cc2)cc1 ; c1ccc(-c2ccc(N(c3ccc(-c4ccc5c6ccc7ccccc7c6n(-c6ccccc6)c5c4)cc3)c3cccc4sc5cc(-c6ccccc6)ccc5c34)cc2)cc1 ; c1ccc(-c2ccc(N(c3ccc(-c4ccc5c6ccc7ccccc7c6n(-c6ccccc6)c5c4)cc3)c3cccc4sc5c6ccccc6ccc5c34)cc2)cc1] 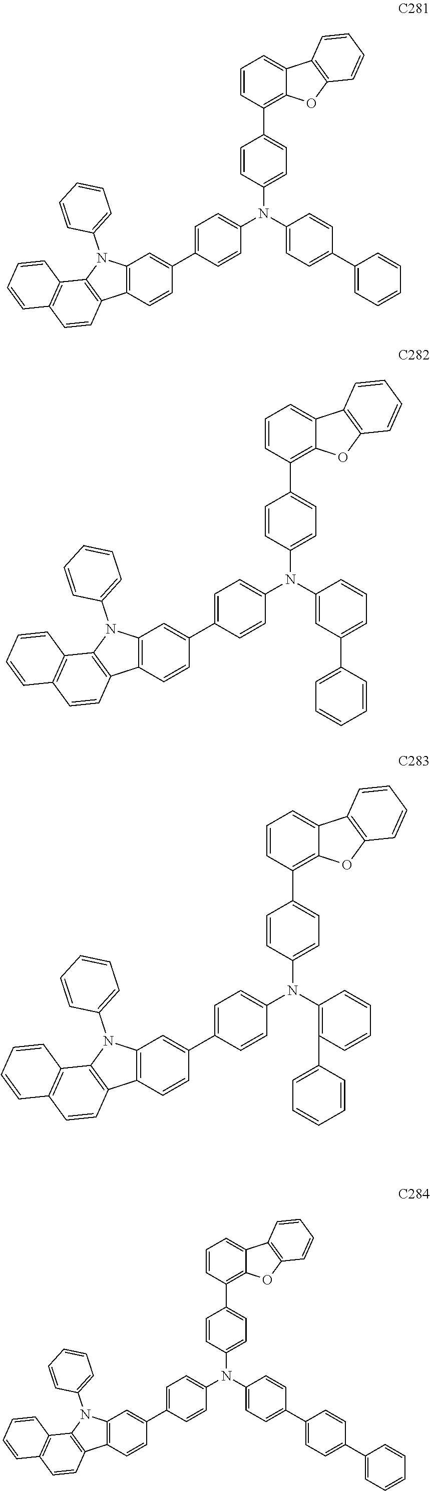 [SMILES: c1ccc(-c2ccc(N(c3ccc(-c4ccc5c6ccc7ccccc7c6n(-c6ccccc6)c5c4)cc3)c3ccc(-c4cccc5c4oc4ccccc45)cc3)cc2)cc1 ; c1ccc(-c2cccc(N(c3ccc(-c4ccc5c6ccc7ccccc7c6n(-c6ccccc6)c5c4)cc3)c3ccc(-c4cccc5c4oc4ccccc45)cc3)c2)cc1 ; c1ccc(-c2ccccc2N(c2ccc(-c3ccc4c5ccc6ccccc6c5n(-c5ccccc5)c4c3)cc2)c2ccc(-c3cccc4c3oc3ccccc34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6c5oc5ccccc56)cc4)cc3)cc2)cc1] 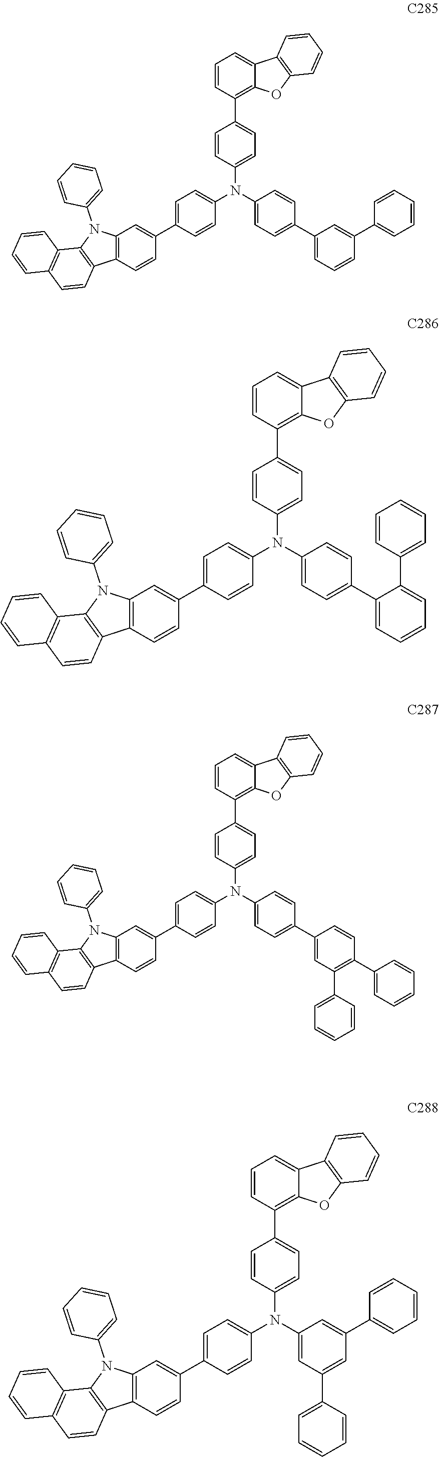 [SMILES: c1ccc(-c2cccc(-c3ccc(N(c4ccc(-c5ccc6c7ccc8ccccc8c7n(-c7ccccc7)c6c5)cc4)c4ccc(-c5cccc6c5oc5ccccc56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5c4oc4ccccc45)cc3)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6c5oc5ccccc56)cc4)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cccc5c4oc4ccccc45)cc3)c2)cc1] 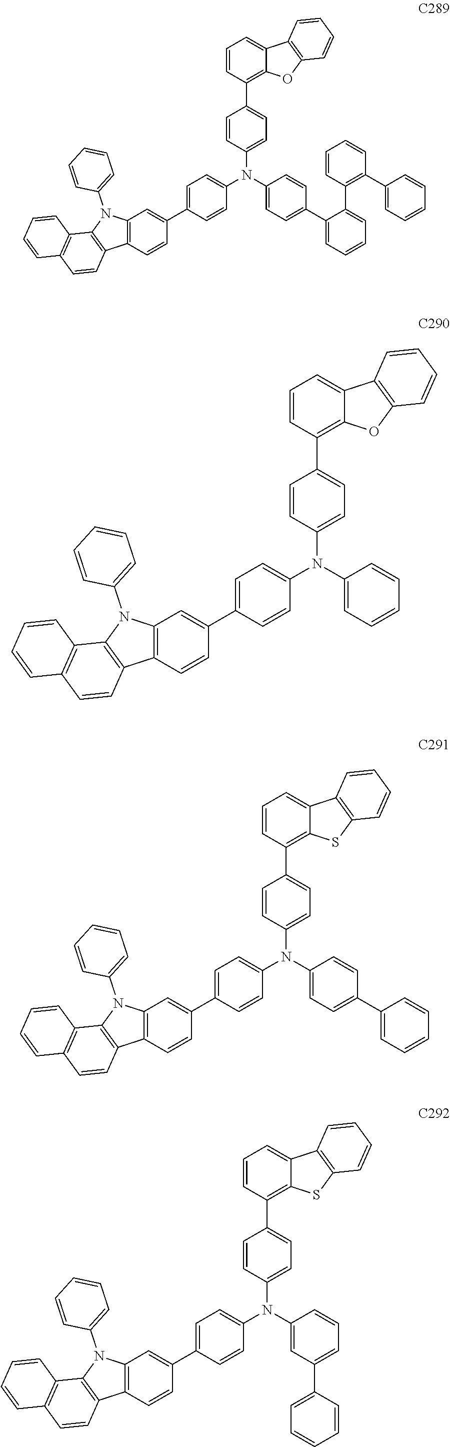 [SMILES: c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5c4oc4ccccc45)cc3)cc2)cc1 ; c1ccc(N(c2ccc(-c3ccc4c5ccc6ccccc6c5n(-c5ccccc5)c4c3)cc2)c2ccc(-c3cccc4c3oc3ccccc34)cc2)cc1 ; c1ccc(-c2ccc(N(c3ccc(-c4ccc5c6ccc7ccccc7c6n(-c6ccccc6)c5c4)cc3)c3ccc(-c4cccc5c4sc4ccccc45)cc3)cc2)cc1 ; c1ccc(-c2cccc(N(c3ccc(-c4ccc5c6ccc7ccccc7c6n(-c6ccccc6)c5c4)cc3)c3ccc(-c4cccc5c4sc4ccccc45)cc3)c2)cc1] 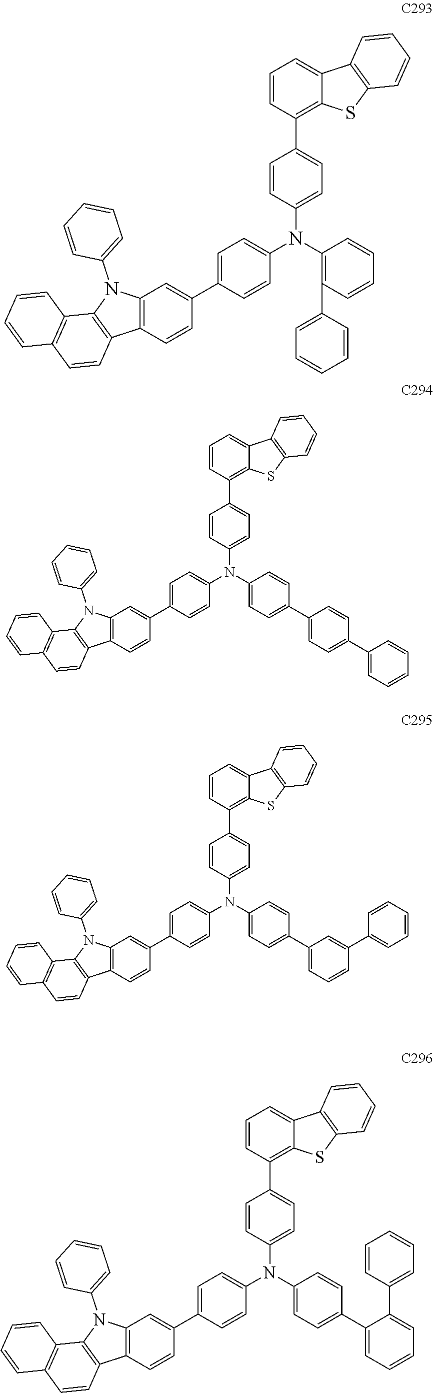 [SMILES: c1ccc(-c2ccccc2N(c2ccc(-c3ccc4c5ccc6ccccc6c5n(-c5ccccc5)c4c3)cc2)c2ccc(-c3cccc4c3sc3ccccc34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6c5sc5ccccc56)cc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc(-c5cccc6c5sc5ccccc56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5c4sc4ccccc45)cc3)cc2)cc1] 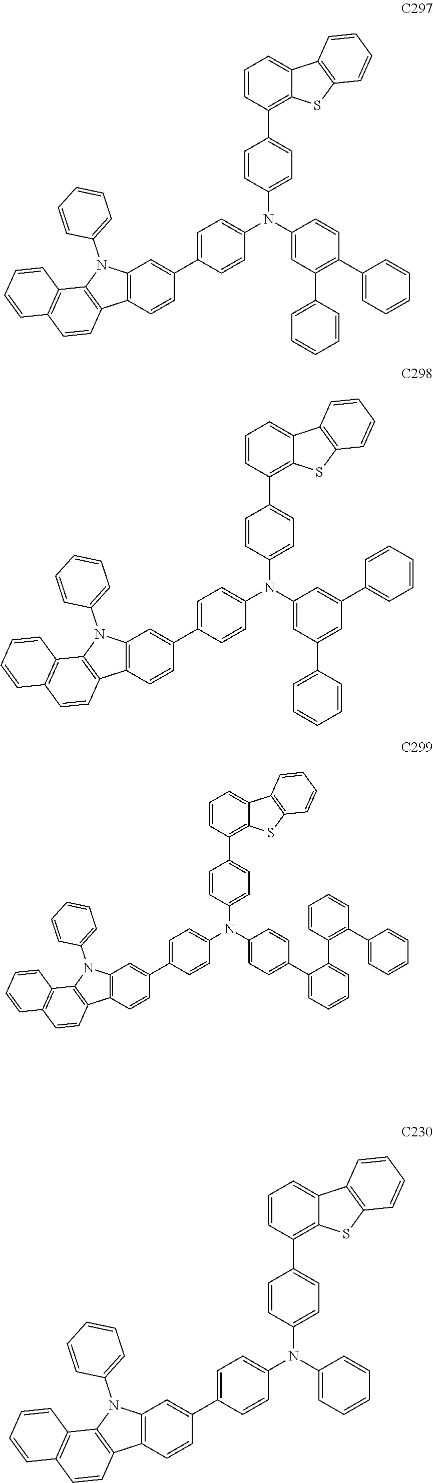 [SMILES: c1ccc(-c2ccc(N(c3ccc(-c4ccc5c6ccc7ccccc7c6n(-c6ccccc6)c5c4)cc3)c3ccc(-c4cccc5c4sc4ccccc45)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cccc5c4sc4ccccc45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5c4sc4ccccc45)cc3)cc2)cc1 ; c1ccc(N(c2ccc(-c3ccc4c5ccc6ccccc6c5n(-c5ccccc5)c4c3)cc2)c2ccc(-c3cccc4c3sc3ccccc34)cc2)cc1] 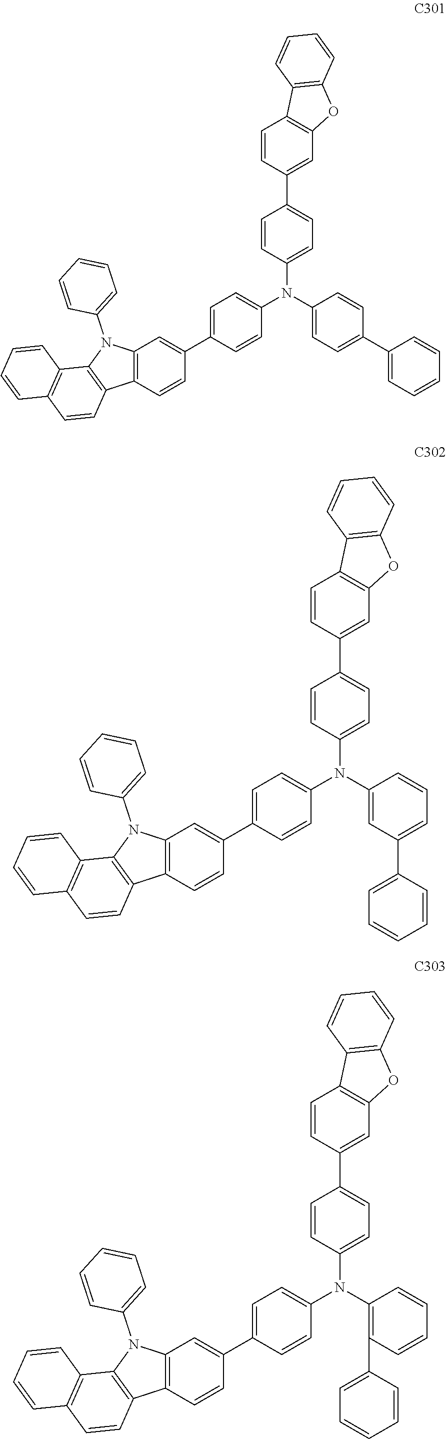 [SMILES: c1ccc(-c2ccc(N(c3ccc(-c4ccc5c(c4)oc4ccccc45)cc3)c3ccc(-c4ccc5c6ccc7ccccc7c6n(-c6ccccc6)c5c4)cc3)cc2)cc1 ; c1ccc(-c2cccc(N(c3ccc(-c4ccc5c(c4)oc4ccccc45)cc3)c3ccc(-c4ccc5c6ccc7ccccc7c6n(-c6ccccc6)c5c4)cc3)c2)cc1 ; c1ccc(-c2ccccc2N(c2ccc(-c3ccc4c(c3)oc3ccccc34)cc2)c2ccc(-c3ccc4c5ccc6ccccc6c5n(-c5ccccc5)c4c3)cc2)cc1] 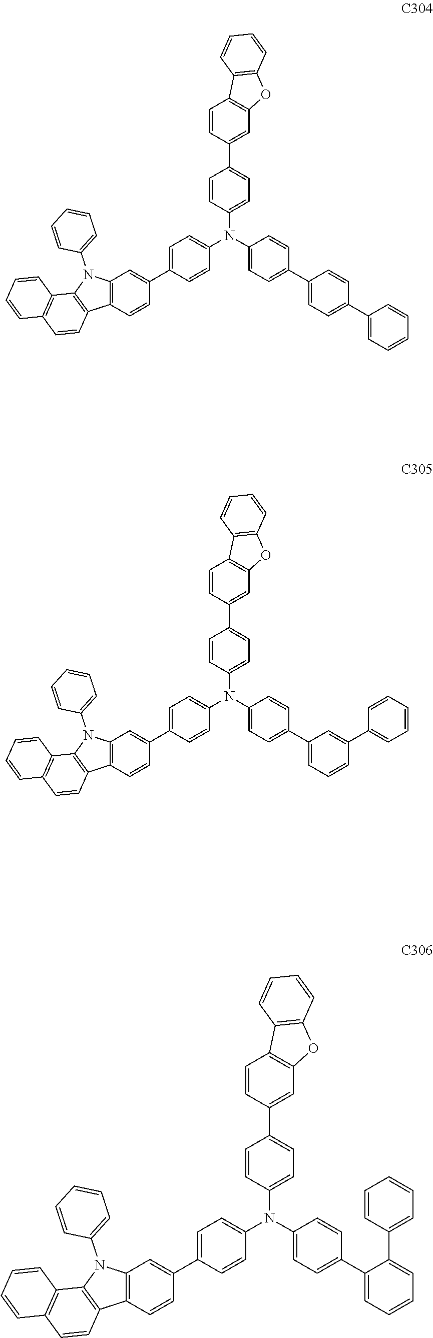 [SMILES: c1ccc(-c2ccc(-c3ccc(N(c4ccc(-c5ccc6c(c5)oc5ccccc56)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6c(c5)oc5ccccc56)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5c(c4)oc4ccccc45)cc3)c3ccc(-c4ccc5c6ccc7ccccc7c6n(-c6ccccc6)c5c4)cc3)cc2)cc1] 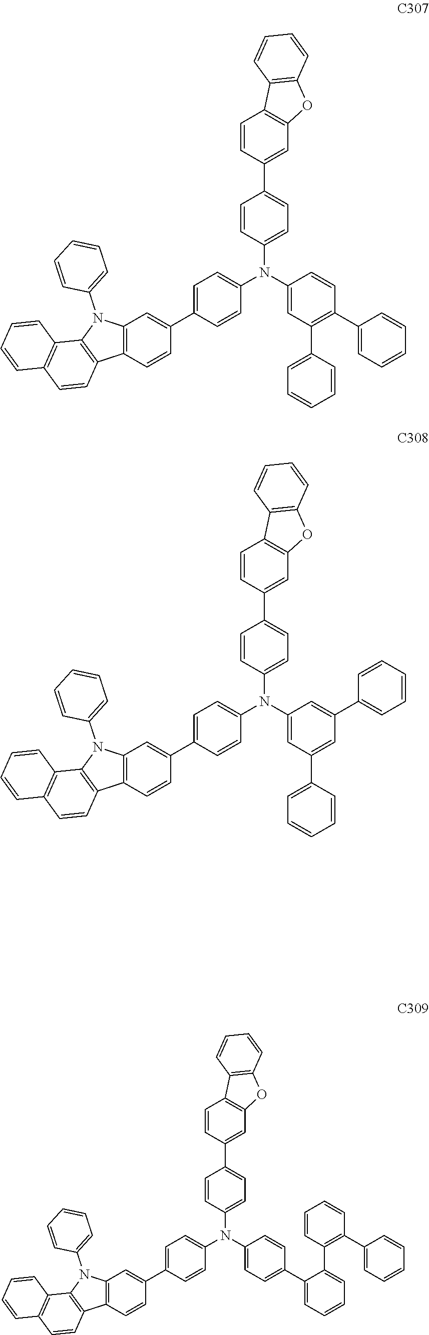 [SMILES: c1ccc(-c2ccc(N(c3ccc(-c4ccc5c(c4)oc4ccccc45)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5c(c4)oc4ccccc45)cc3)c3ccc(-c4ccc5c6ccc7ccccc7c6n(-c6ccccc6)c5c4)cc3)c2)cc1 ; c1ccc(-c2ccccc2-c2ccccc2-c2ccc(N(c3ccc(-c4ccc5c(c4)oc4ccccc45)cc3)c3ccc(-c4ccc5c6ccc7ccccc7c6n(-c6ccccc6)c5c4)cc3)cc2)cc1] 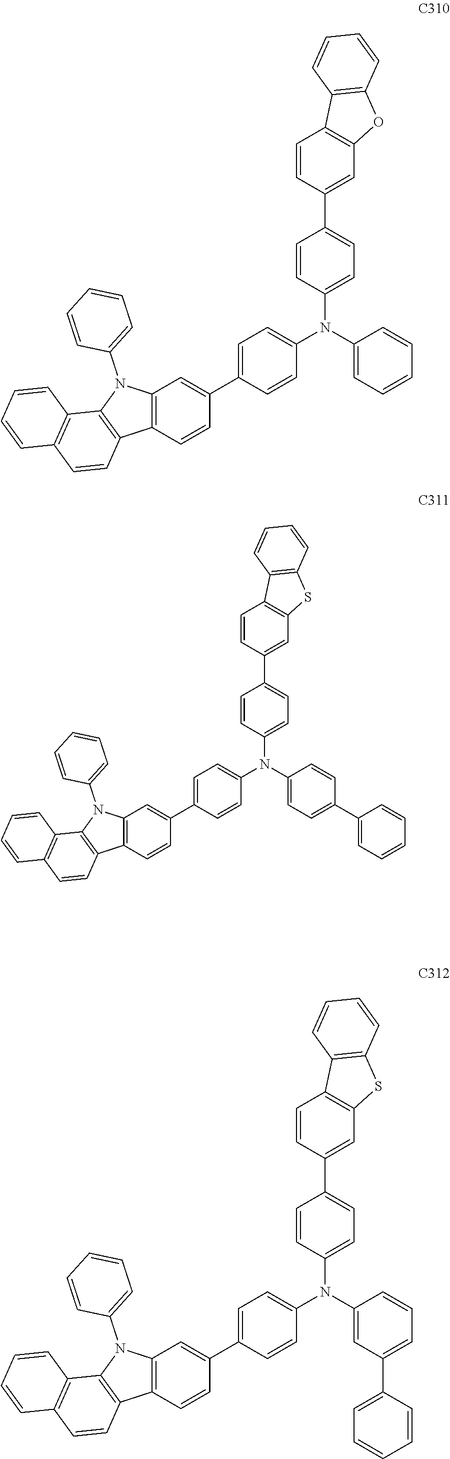 [SMILES: c1ccc(N(c2ccc(-c3ccc4c(c3)oc3ccccc34)cc2)c2ccc(-c3ccc4c5ccc6ccccc6c5n(-c5ccccc5)c4c3)cc2)cc1 ; c1ccc(-c2ccc(N(c3ccc(-c4ccc5c(c4)sc4ccccc45)cc3)c3ccc(-c4ccc5c6ccc7ccccc7c6n(-c6ccccc6)c5c4)cc3)cc2)cc1 ; c1ccc(-c2cccc(N(c3ccc(-c4ccc5c(c4)sc4ccccc45)cc3)c3ccc(-c4ccc5c6ccc7ccccc7c6n(-c6ccccc6)c5c4)cc3)c2)cc1] 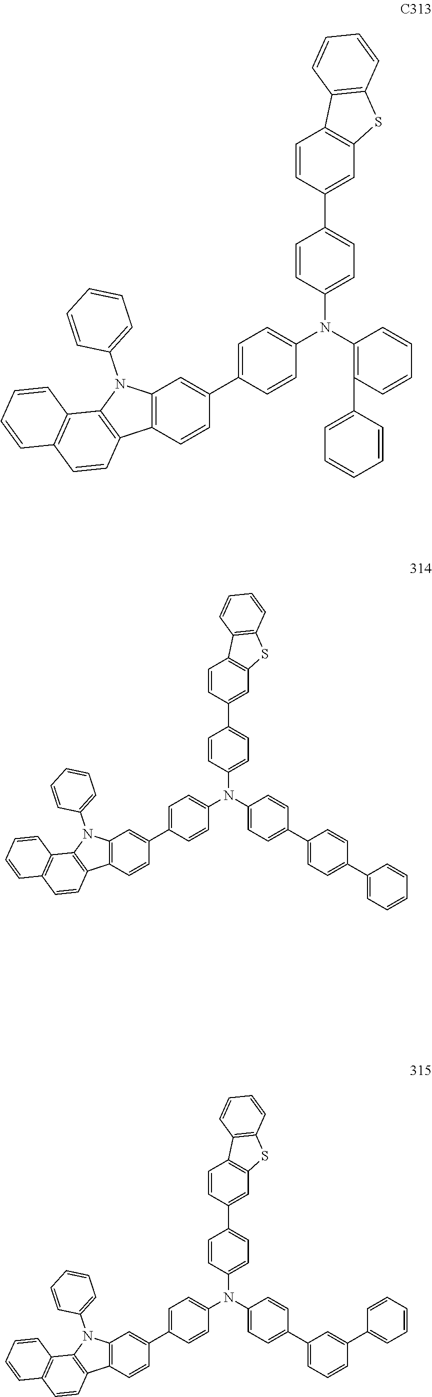 [SMILES: c1ccc(-c2ccccc2N(c2ccc(-c3ccc4c(c3)sc3ccccc34)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6c(c5)sc5ccccc56)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6c(c5)sc5ccccc56)cc4)c4ccc(-c5ccc6c7ccc8ccccc8c7n(-c7ccccc7)c6c5)cc4)cc3)c2)cc1] 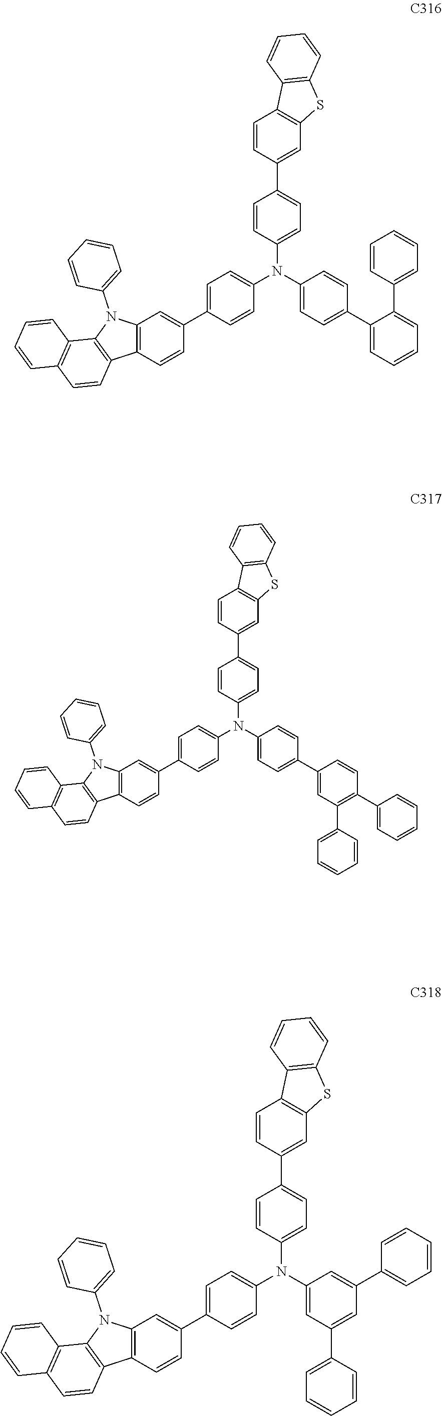 [SMILES: c1ccc(-c2ccccc2-c2ccc(N(c3ccc(-c4ccc5c(c4)sc4ccccc45)cc3)c3ccc(-c4ccc5c6ccc7ccccc7c6n(-c6ccccc6)c5c4)cc3)cc2)cc1 ; c1ccc(-c2ccc(-c3ccc(N(c4ccc(-c5ccc6c(c5)sc5ccccc56)cc4)c4ccc(-c5ccc6c7ccc8ccccc8c7n(-c7ccccc7)c6c5)cc4)cc3)cc2-c2ccccc2)cc1 ; c1ccc(-c2cc(-c3ccccc3)cc(N(c3ccc(-c4ccc5c(c4)sc4ccccc45)cc3)c3ccc(-c4ccc5c6ccc7ccccc7c6n(-c6ccccc6)c5c4)cc3)c2)cc1] 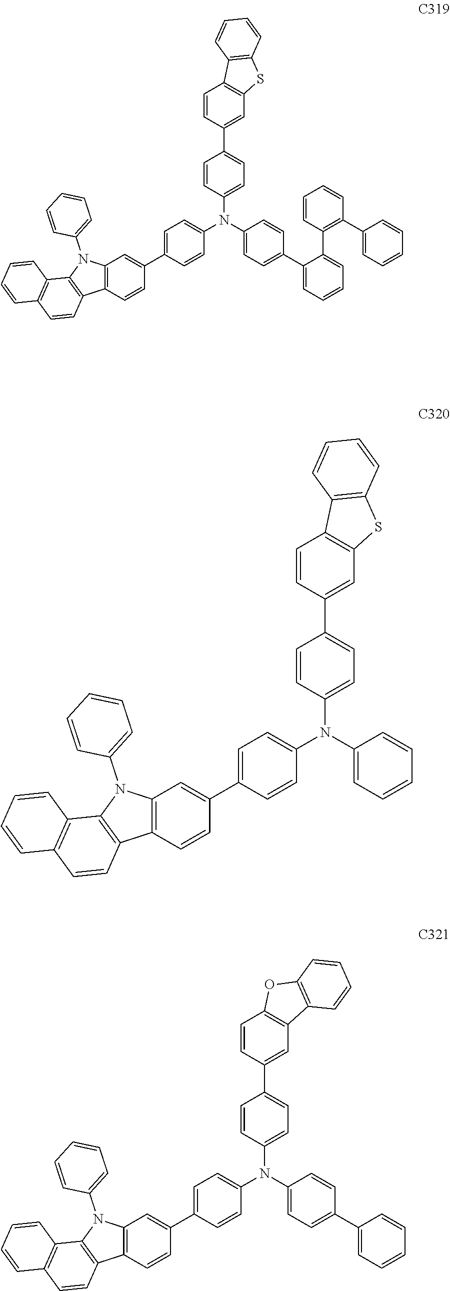 [SMILES: c1ccc(-c2ccccc2-c2ccccc2-c2ccc(N(c3ccc(-c4ccc5c(c4)sc4ccccc45)cc3)c3ccc(-c4ccc5c6ccc7ccccc7c6n(-c6ccccc6)c5c4)cc3)cc2)cc1 ; c1ccc(N(c2ccc(-c3ccc4c(c3)sc3ccccc34)cc2)c2ccc(-c3ccc4c5ccc6ccccc6c5n(-c5ccccc5)c4c3)cc2)cc1 ; c1ccc(-c2ccc(N(c3ccc(-c4ccc5oc6ccccc6c5c4)cc3)c3ccc(-c4ccc5c6ccc7ccccc7c6n(-c6ccccc6)c5c4)cc3)cc2)cc1] 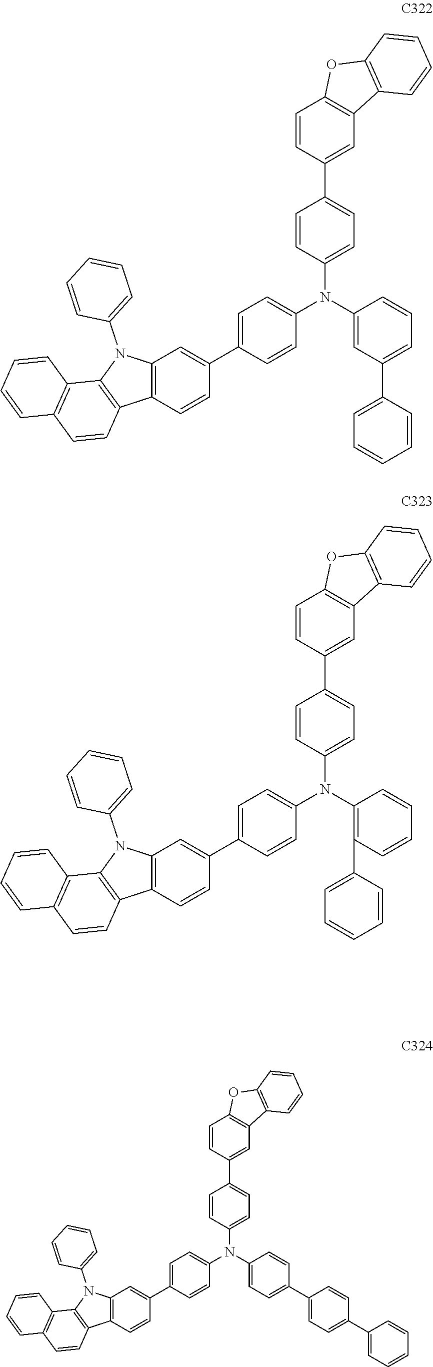 [SMILES: c1ccc(-c2cccc(N(c3ccc(-c4ccc5oc6ccccc6c5c4)cc3)c3ccc(-c4ccc5c6ccc7ccccc7c6n(-c6ccccc6)c5c4)cc3)c2)cc1 ; c1ccc(-c2ccccc2N(c2ccc(-c3ccc4oc5ccccc5c4c3)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6oc7ccccc7c6c5)cc4)c4ccc(-c5ccc6c7ccc8ccccc8c7n(-c7ccccc7)c6c5)cc4)cc3)cc2)cc1] 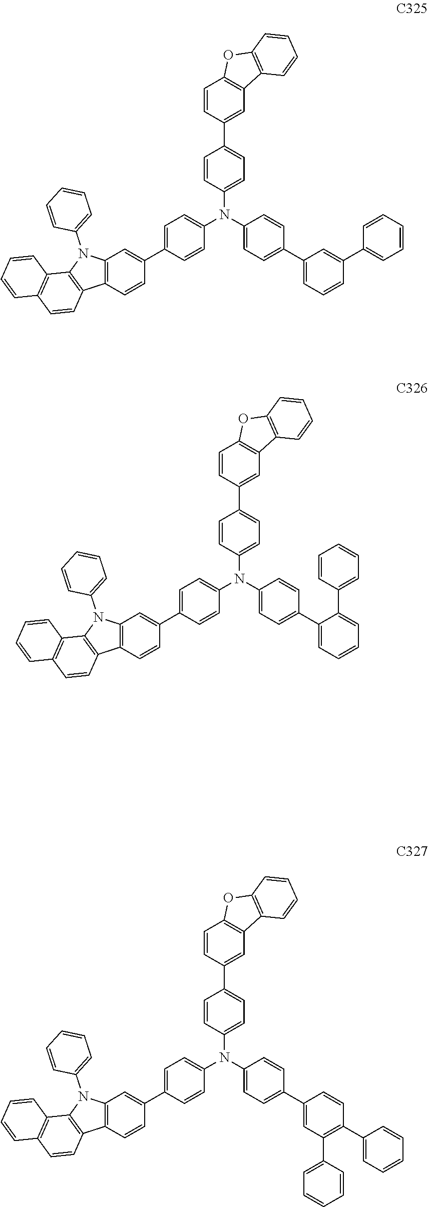 [SMILES: c1ccc(-c2cccc(-c3ccc(N(c4ccc(-c5ccc6oc7ccccc7c6c5)cc4)c4ccc(-c5ccc6c7ccc8ccccc8c7n(-c7ccccc7)c6c5)cc4)cc3)c2)cc1 ; c1ccc(-c2ccccc2-c2ccc(N(c3ccc(-c4ccc5oc6ccccc6c5c4)cc3)c3ccc(-c4ccc5c6ccc7ccccc7c6n(-c6ccccc6)c5c4)cc3)cc2)cc1 ; c1ccc(-c2ccc(-c3ccc(N(c4ccc(-c5ccc6oc7ccccc7c6c5)cc4)c4ccc(-c5ccc6c7ccc8ccccc8c7n(-c7ccccc7)c6c5)cc4)cc3)cc2-c2ccccc2)cc1] 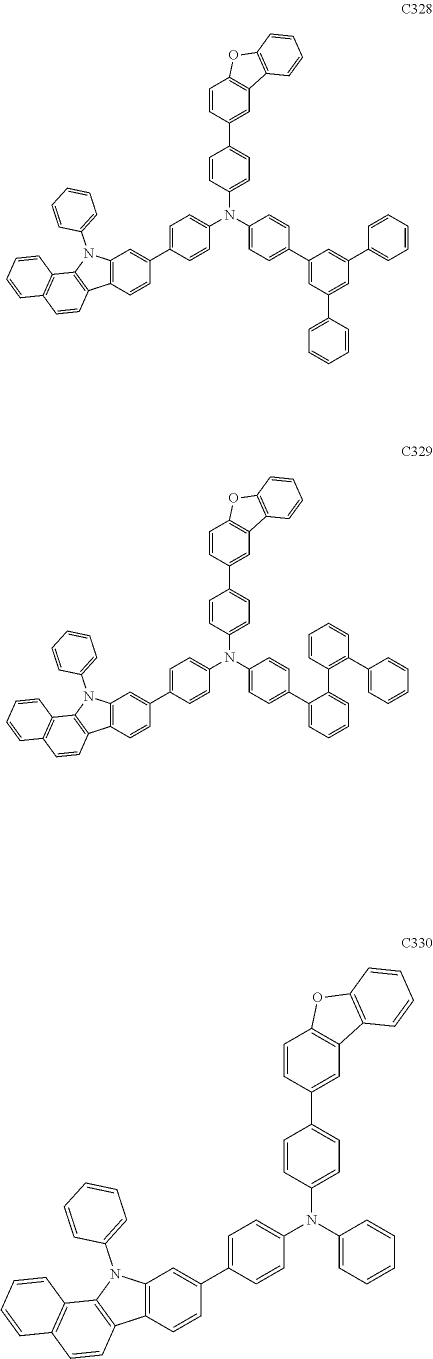 [SMILES: c1ccc(-c2cc(-c3ccccc3)cc(-c3ccc(N(c4ccc(-c5ccc6oc7ccccc7c6c5)cc4)c4ccc(-c5ccc6c7ccc8ccccc8c7n(-c7ccccc7)c6c5)cc4)cc3)c2)cc1 ; c1ccc(-c2ccccc2-c2ccccc2-c2ccc(N(c3ccc(-c4ccc5oc6ccccc6c5c4)cc3)c3ccc(-c4ccc5c6ccc7ccccc7c6n(-c6ccccc6)c5c4)cc3)cc2)cc1 ; c1ccc(N(c2ccc(-c3ccc4oc5ccccc5c4c3)cc2)c2ccc(-c3ccc4c5ccc6ccccc6c5n(-c5ccccc5)c4c3)cc2)cc1] 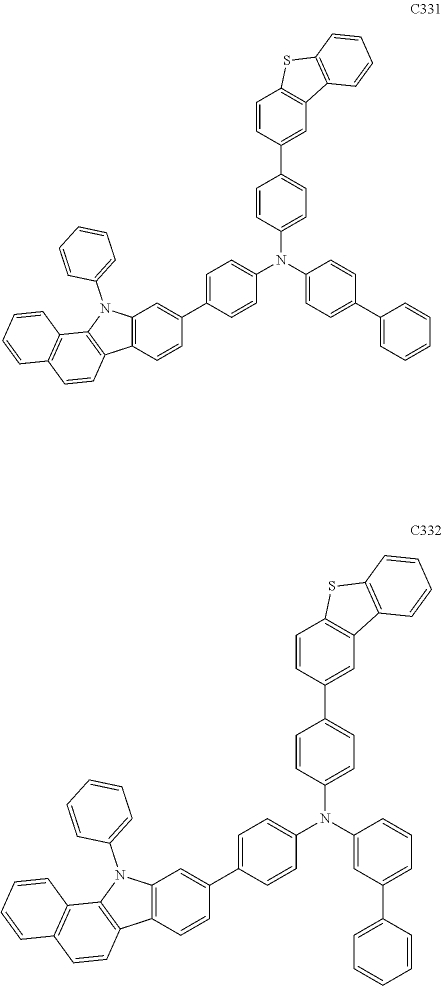 [SMILES: c1ccc(-c2ccc(N(c3ccc(-c4ccc5sc6ccccc6c5c4)cc3)c3ccc(-c4ccc5c6ccc7ccccc7c6n(-c6ccccc6)c5c4)cc3)cc2)cc1 ; c1ccc(-c2cccc(N(c3ccc(-c4ccc5sc6ccccc6c5c4)cc3)c3ccc(-c4ccc5c6ccc7ccccc7c6n(-c6ccccc6)c5c4)cc3)c2)cc1] 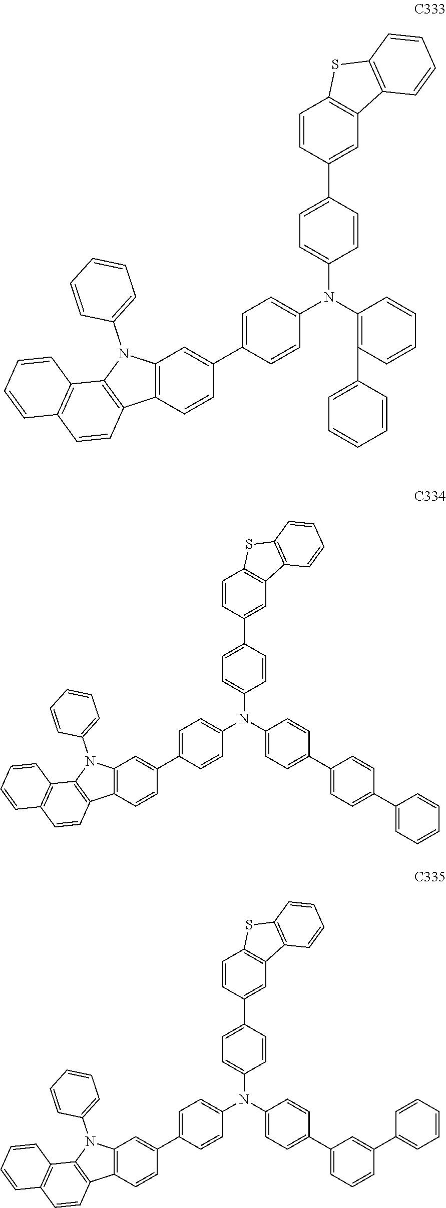 [SMILES: c1ccc(-c2ccccc2N(c2ccc(-c3ccc4sc5ccccc5c4c3)cc2)c2ccc(-c3ccc4c5ccc6ccccc6c5n(-c5ccccc5)c4c3)cc2)cc1 ; c1ccc(-c2ccc(-c3ccc(N(c4ccc(-c5ccc6sc7ccccc7c6c5)cc4)c4ccc(-c5ccc6c7ccc8ccccc8c7n(-c7ccccc7)c6c5)cc4)cc3)cc2)cc1 ; c1ccc(-c2cccc(-c3ccc(N(c4ccc(-c5ccc6sc7ccccc7c6c5)cc4)c4ccc(-c5ccc6c7ccc8ccccc8c7n(-c7ccccc7)c6c5)cc4)cc3)c2)cc1] 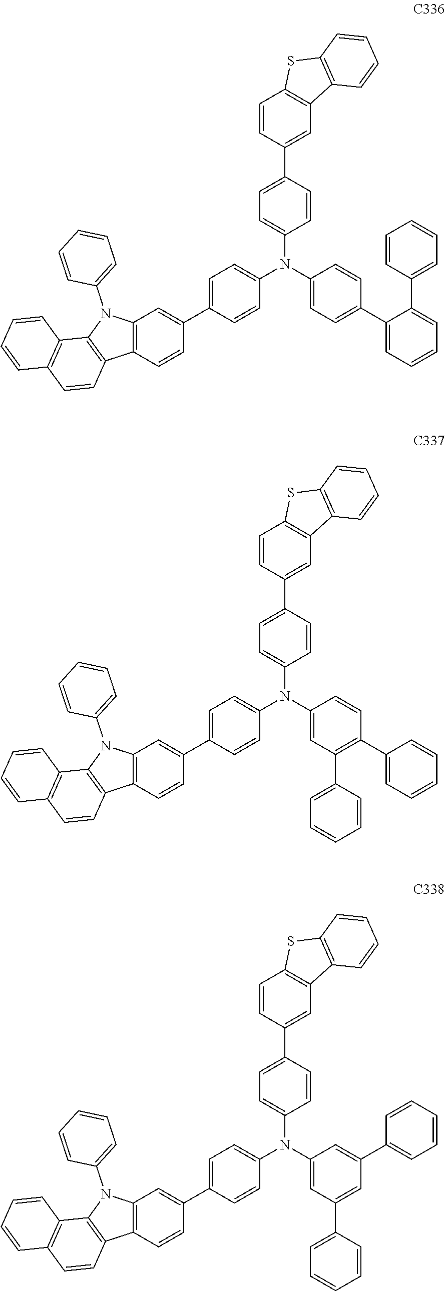 [SMILES: c1ccc(-c2ccccc2-c2ccc(N(c3ccc(-c4ccc5sc6ccccc6c5c4)cc3)c3ccc(-c4ccc5c6ccc7ccccc7c6n(-c6ccccc6)c5c4)cc3)cc2)cc1 ; c1ccc(-c2ccc(N(c3ccc(-c4ccc5sc6ccccc6c5c4)cc3)c3ccc(-c4ccc5c6ccc7ccccc7c6n(-c6ccccc6)c5c4)cc3)cc2-c2ccccc2)cc1 ; c1ccc(-c2cc(-c3ccccc3)cc(N(c3ccc(-c4ccc5sc6ccccc6c5c4)cc3)c3ccc(-c4ccc5c6ccc7ccccc7c6n(-c6ccccc6)c5c4)cc3)c2)cc1] 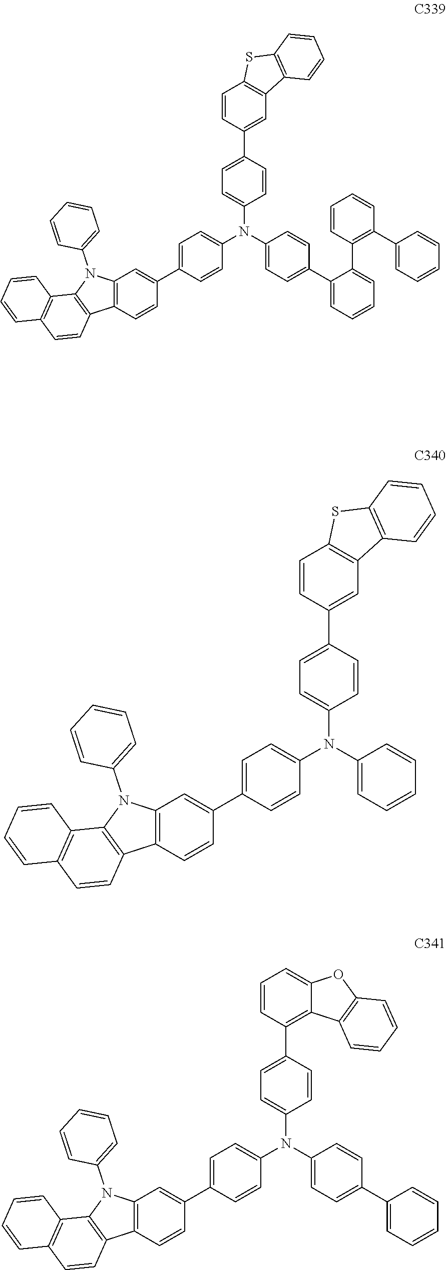 [SMILES: c1ccc(-c2ccccc2-c2ccccc2-c2ccc(N(c3ccc(-c4ccc5sc6ccccc6c5c4)cc3)c3ccc(-c4ccc5c6ccc7ccccc7c6n(-c6ccccc6)c5c4)cc3)cc2)cc1 ; c1ccc(N(c2ccc(-c3ccc4sc5ccccc5c4c3)cc2)c2ccc(-c3ccc4c5ccc6ccccc6c5n(-c5ccccc5)c4c3)cc2)cc1 ; c1ccc(-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1] 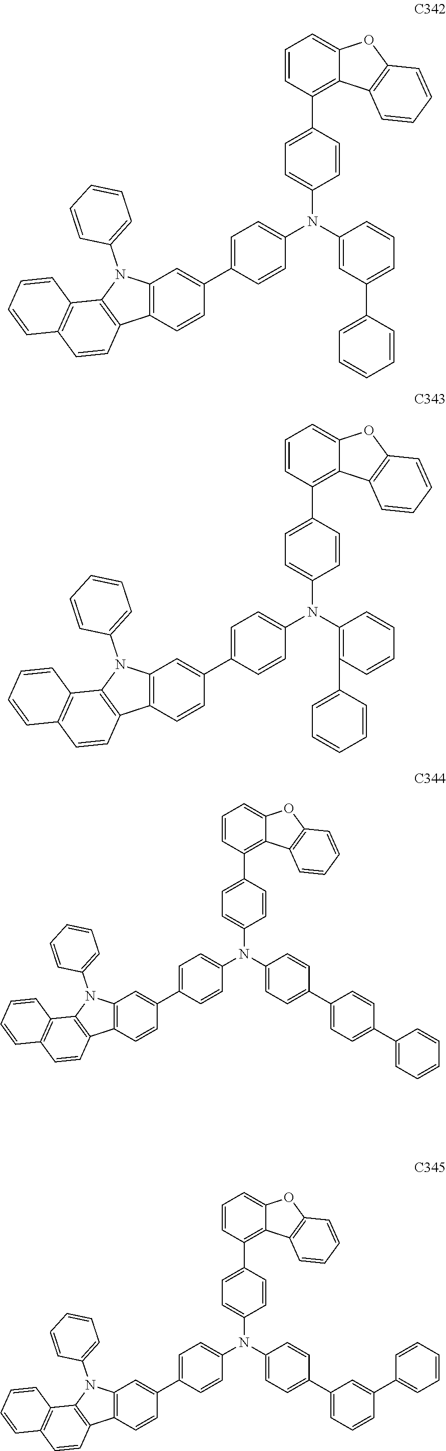 [SMILES: c1ccc(-c2cccc(N(c3ccc(-c4ccc5c6ccc7ccccc7c6n(-c6ccccc6)c5c4)cc3)c3ccc(-c4cccc5oc6ccccc6c45)cc3)c2)cc1 ; c1ccc(-c2ccccc2N(c2ccc(-c3ccc4c5ccc6ccccc6c5n(-c5ccccc5)c4c3)cc2)c2ccc(-c3cccc4oc5ccccc5c34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6oc7ccccc7c56)cc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc(-c5cccc6oc7ccccc7c56)cc4)cc3)c2)cc1] 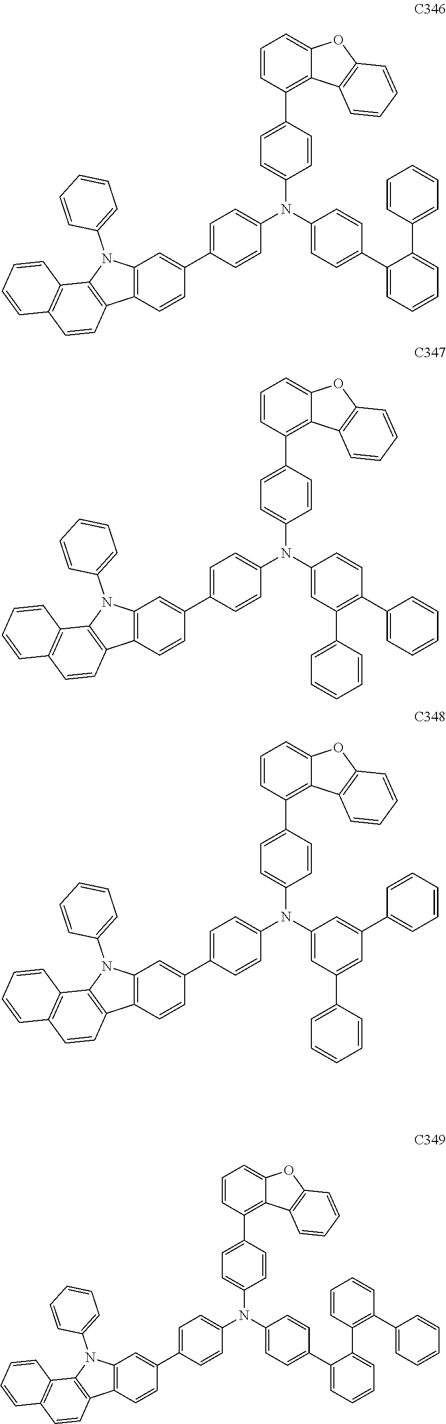 [SMILES: c1ccc(-c2ccccc2-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1 ; c1ccc(-c2ccc(N(c3ccc(-c4ccc5c6ccc7ccccc7c6n(-c6ccccc6)c5c4)cc3)c3ccc(-c4cccc5oc6ccccc6c45)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cccc5oc6ccccc6c45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5oc6ccccc6c45)cc3)cc2)cc1] 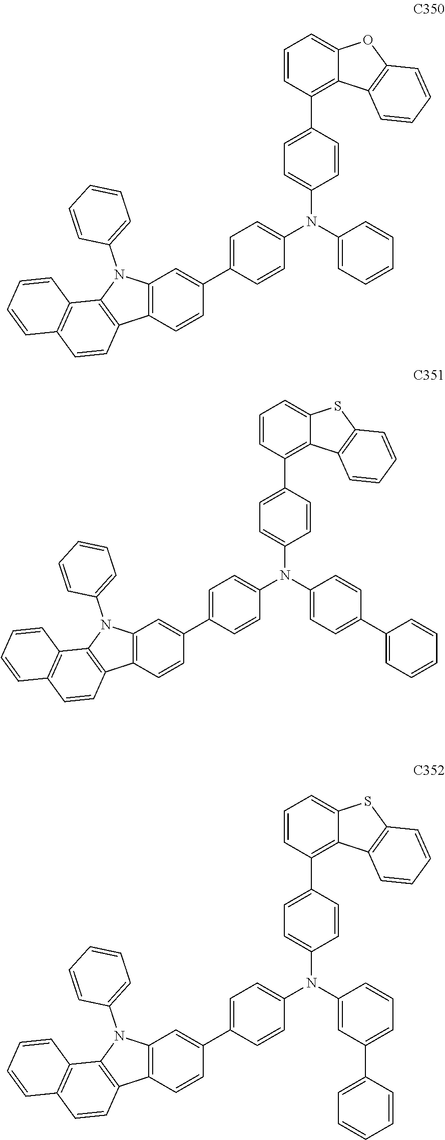 [SMILES: c1ccc(N(c2ccc(-c3ccc4c5ccc6ccccc6c5n(-c5ccccc5)c4c3)cc2)c2ccc(-c3cccc4oc5ccccc5c34)cc2)cc1 ; c1ccc(-c2ccc(N(c3ccc(-c4ccc5c6ccc7ccccc7c6n(-c6ccccc6)c5c4)cc3)c3ccc(-c4cccc5sc6ccccc6c45)cc3)cc2)cc1 ; c1ccc(-c2cccc(N(c3ccc(-c4ccc5c6ccc7ccccc7c6n(-c6ccccc6)c5c4)cc3)c3ccc(-c4cccc5sc6ccccc6c45)cc3)c2)cc1] 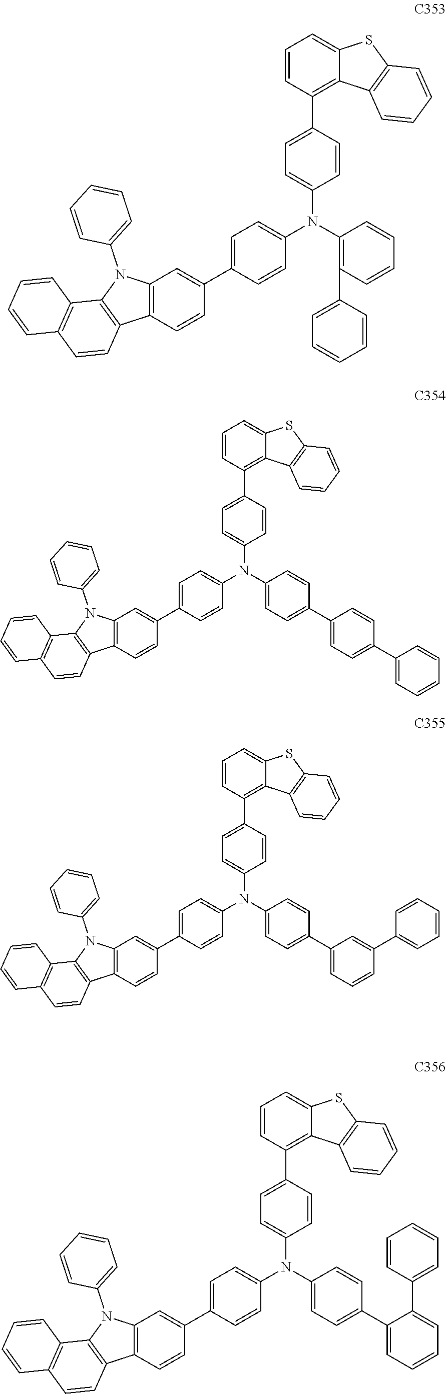 [SMILES: c1ccc(-c2ccccc2N(c2ccc(-c3ccc4c5ccc6ccccc6c5n(-c5ccccc5)c4c3)cc2)c2ccc(-c3cccc4sc5ccccc5c34)cc2)cc1 ; c1ccc(-c2ccc(-c3ccc(N(c4ccc(-c5ccc6c7ccc8ccccc8c7n(-c7ccccc7)c6c5)cc4)c4ccc(-c5cccc6sc7ccccc7c56)cc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc(-c5cccc6sc7ccccc7c56)cc4)cc3)c2)cc1 ; c1ccc(-c2ccccc2-c2ccc(N(c3ccc(-c4ccc5c6ccc7ccccc7c6n(-c6ccccc6)c5c4)cc3)c3ccc(-c4cccc5sc6ccccc6c45)cc3)cc2)cc1] 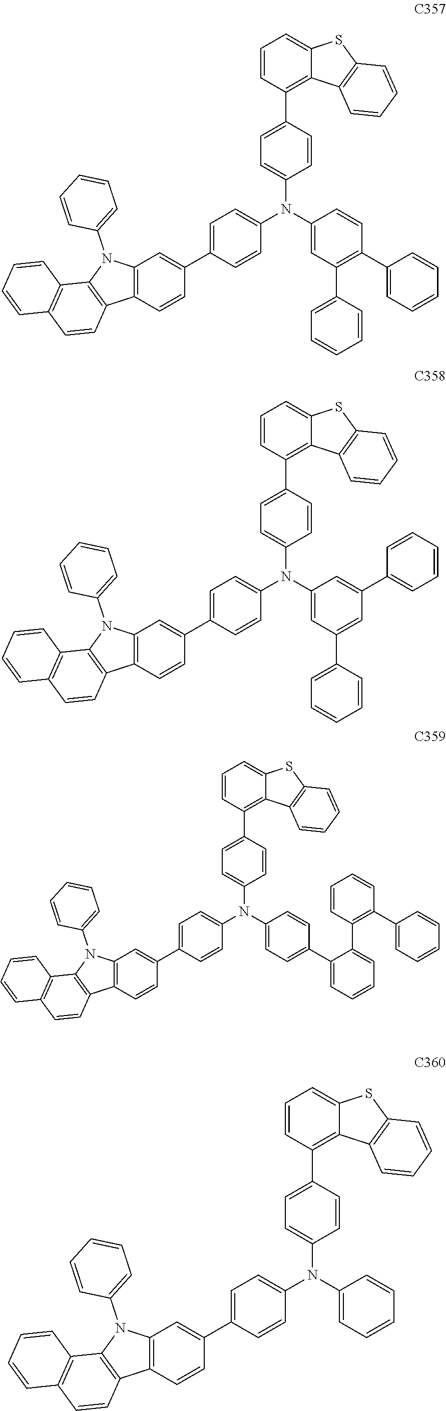 [SMILES: c1ccc(-c2ccc(N(c3ccc(-c4ccc5c6ccc7ccccc7c6n(-c6ccccc6)c5c4)cc3)c3ccc(-c4cccc5sc6ccccc6c45)cc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc(-c4cccc5sc6ccccc6c45)cc3)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc(-c4cccc5sc6ccccc6c45)cc3)cc2)cc1 ; c1ccc(N(c2ccc(-c3ccc4c5ccc6ccccc6c5n(-c5ccccc5)c4c3)cc2)c2ccc(-c3cccc4sc5ccccc5c34)cc2)cc1] 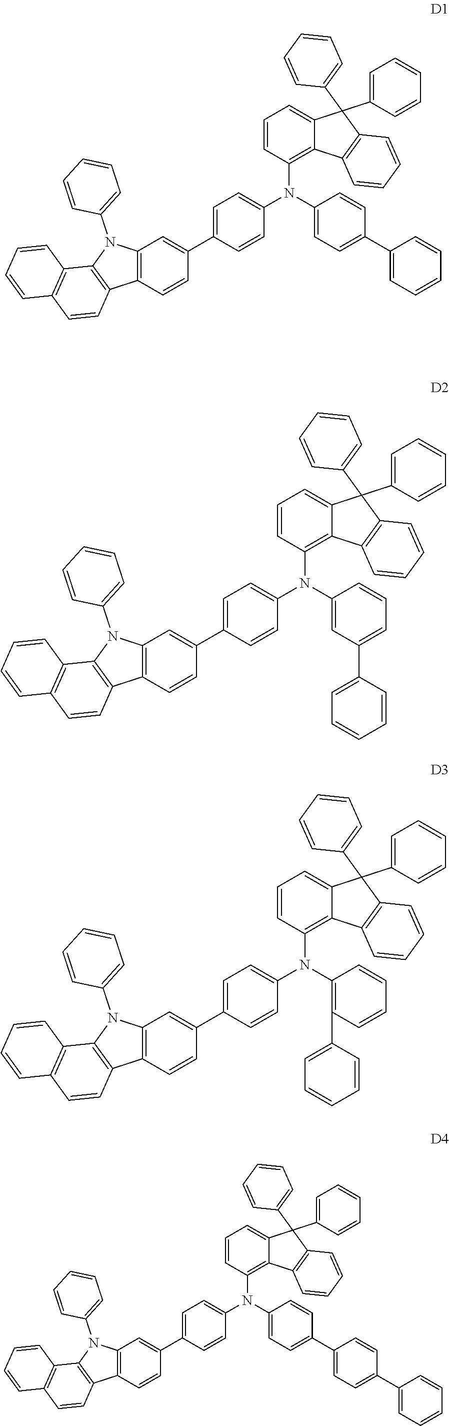 [SMILES: c1ccc(-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(-c2cccc(N(c3ccc(-c4ccc5c6ccc7ccccc7c6n(-c6ccccc6)c5c4)cc3)c3cccc4c3-c3ccccc3C4(c3ccccc3)c3ccccc3)c2)cc1 ; c1ccc(-c2ccccc2N(c2ccc(-c3ccc4c5ccc6ccccc6c5n(-c5ccccc5)c4c3)cc2)c2cccc3c2-c2ccccc2C3(c2ccccc2)c2ccccc2)cc1 ; c1ccc(-c2ccc(-c3ccc(N(c4ccc(-c5ccc6c7ccc8ccccc8c7n(-c7ccccc7)c6c5)cc4)c4cccc5c4-c4ccccc4C5(c4ccccc4)c4ccccc4)cc3)cc2)cc1] 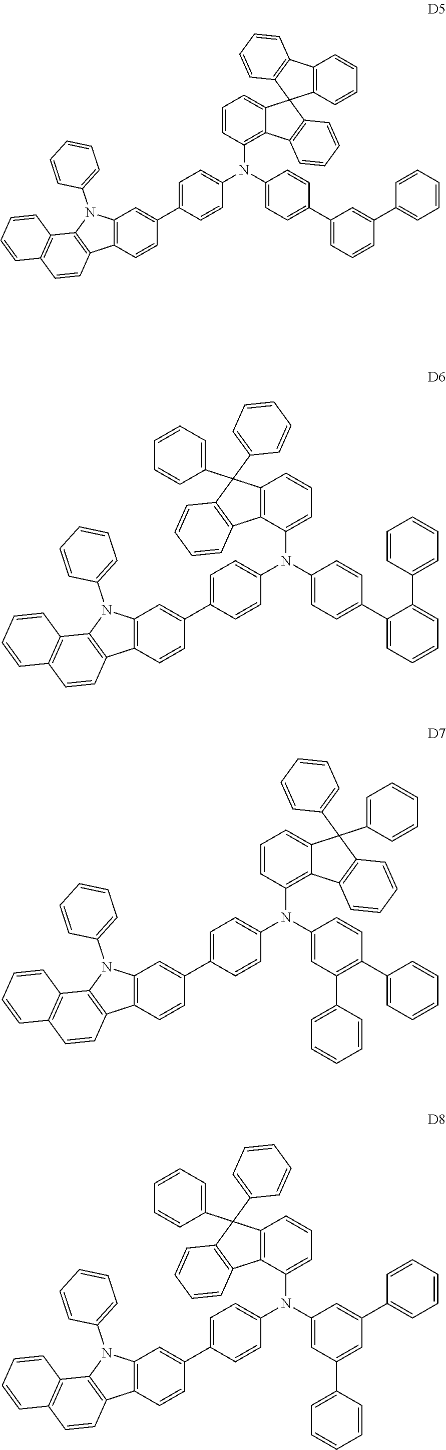 [SMILES: c1ccc(-c2cccc(-c3ccc(N(c4ccc(-c5ccc6c7ccc8ccccc8c7n(-c7ccccc7)c6c5)cc4)c4cccc5c4-c4ccccc4C54c5ccccc5-c5ccccc54)cc3)c2)cc1 ; c1ccc(-c2ccccc2-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4c3-c3ccccc3C4(c3ccccc3)c3ccccc3)c2)cc1] 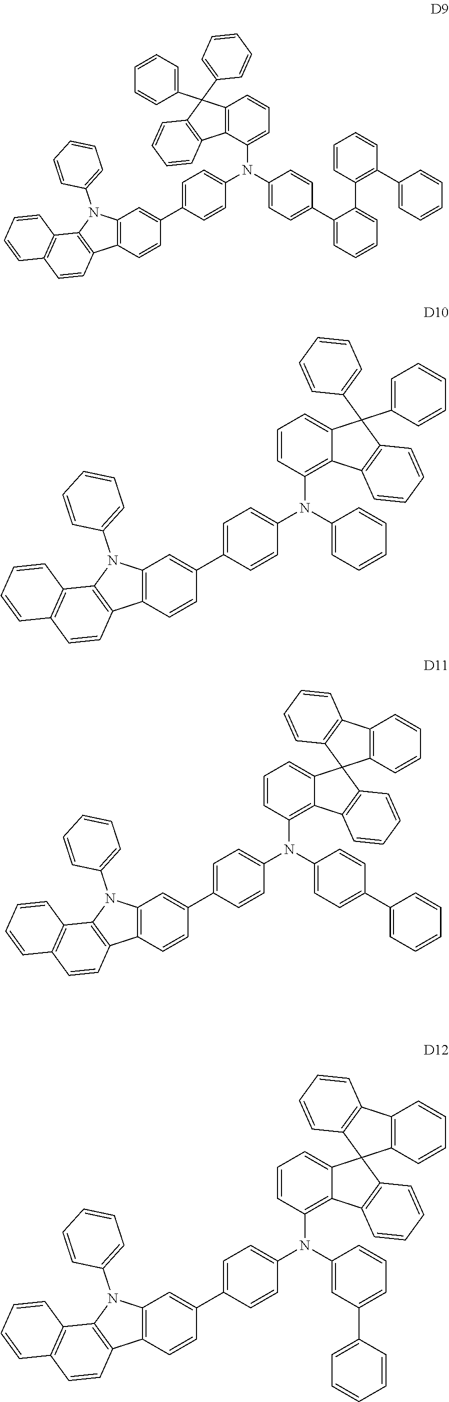 [SMILES: c1ccc(-c2ccccc2-c2ccccc2-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1 ; c1ccc(N(c2ccc(-c3ccc4c5ccc6ccccc6c5n(-c5ccccc5)c4c3)cc2)c2cccc3c2-c2ccccc2C3(c2ccccc2)c2ccccc2)cc1 ; c1ccc(-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1 ; c1ccc(-c2cccc(N(c3ccc(-c4ccc5c6ccc7ccccc7c6n(-c6ccccc6)c5c4)cc3)c3cccc4c3-c3ccccc3C43c4ccccc4-c4ccccc43)c2)cc1] 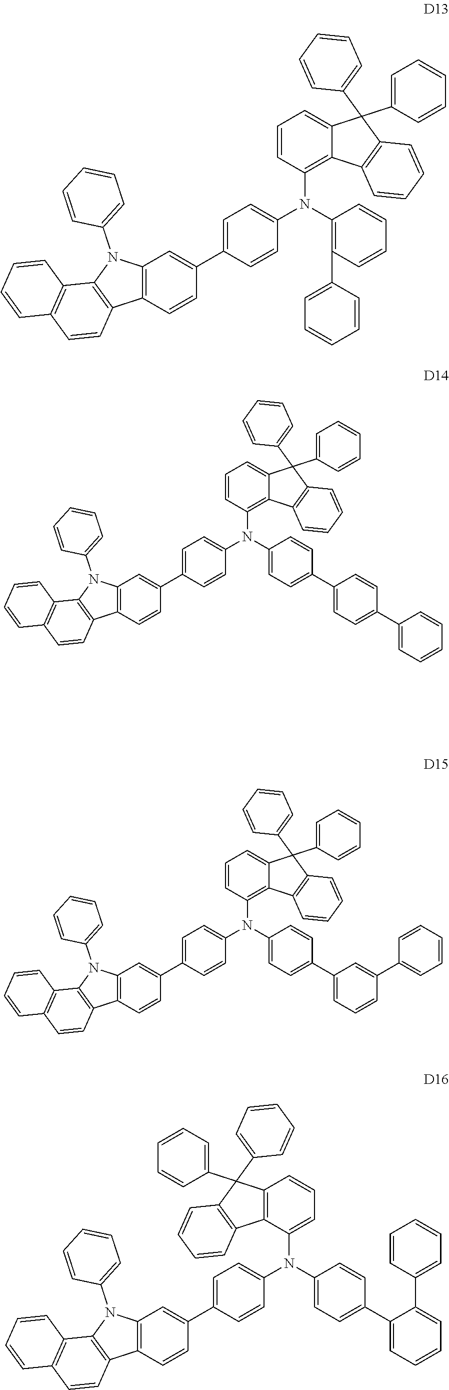 [SMILES: c1ccc(-c2ccccc2N(c2ccc(-c3ccc4c5ccc6ccccc6c5n(-c5ccccc5)c4c3)cc2)c2cccc3c2-c2ccccc2C3(c2ccccc2)c2ccccc2)cc1 ; c1ccc(-c2ccc(-c3ccc(N(c4ccc(-c5ccc6c7ccc8ccccc8c7n(-c7ccccc7)c6c5)cc4)c4cccc5c4-c4ccccc4C5(c4ccccc4)c4ccccc4)cc3)cc2)cc1 ; c1ccc(-c2cccc(-c3ccc(N(c4ccc(-c5ccc6c7ccc8ccccc8c7n(-c7ccccc7)c6c5)cc4)c4cccc5c4-c4ccccc4C5(c4ccccc4)c4ccccc4)cc3)c2)cc1 ; c1ccc(-c2ccccc2-c2ccc(N(c3ccc(-c4ccc5c6ccc7ccccc7c6n(-c6ccccc6)c5c4)cc3)c3cccc4c3-c3ccccc3C4(c3ccccc3)c3ccccc3)cc2)cc1] 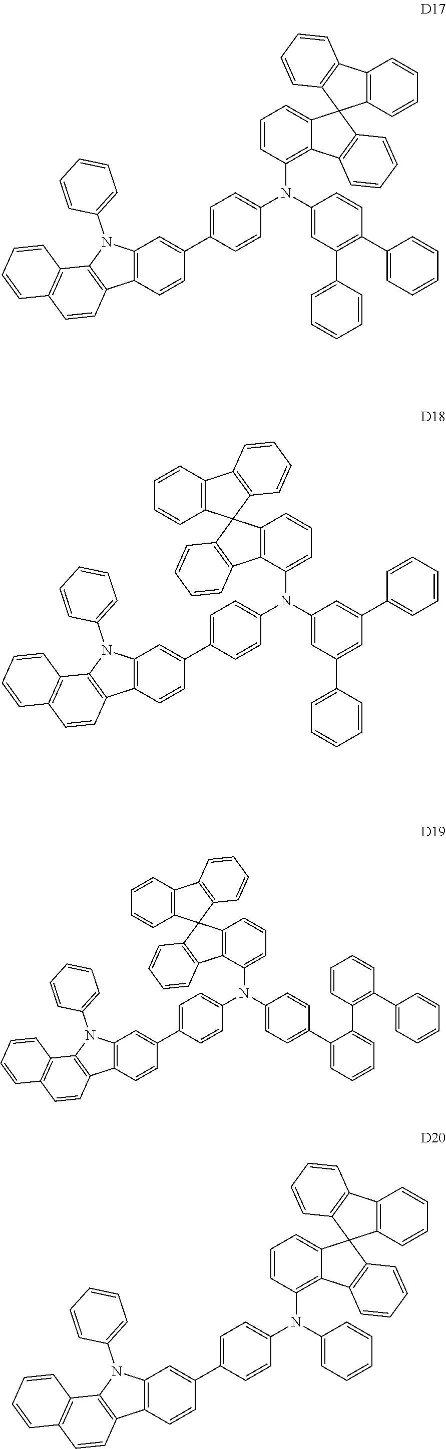 [SMILES: c1ccc(-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3cccc4c3-c3ccccc3C43c4ccccc4-c4ccccc43)c2)cc1 ; c1ccc(-c2ccccc2-c2ccccc2-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1 ; c1ccc(N(c2ccc(-c3ccc4c5ccc6ccccc6c5n(-c5ccccc5)c4c3)cc2)c2cccc3c2-c2ccccc2C32c3ccccc3-c3ccccc32)cc1] 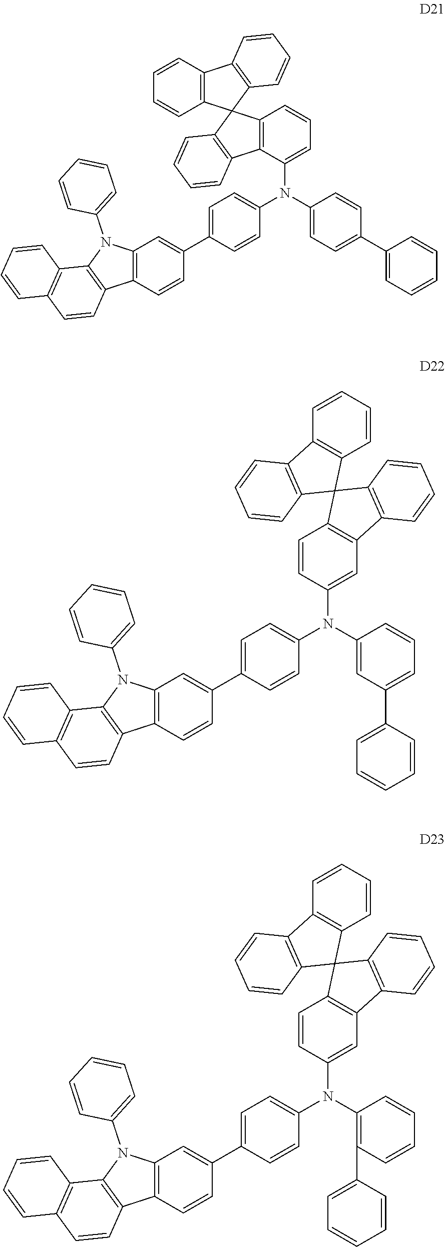 [SMILES: c1ccc(-c2ccc(N(c3ccc(-c4ccc5c6ccc7ccccc7c6n(-c6ccccc6)c5c4)cc3)c3cccc4c3-c3ccccc3C43c4ccccc4-c4ccccc43)cc2)cc1 ; c1ccc(-c2cccc(N(c3ccc(-c4ccc5c6ccc7ccccc7c6n(-c6ccccc6)c5c4)cc3)c3ccc4c(c3)-c3ccccc3C43c4ccccc4-c4ccccc43)c2)cc1 ; c1ccc(-c2ccccc2N(c2ccc(-c3ccc4c5ccc6ccccc6c5n(-c5ccccc5)c4c3)cc2)c2ccc3c(c2)-c2ccccc2C32c3ccccc3-c3ccccc32)cc1] 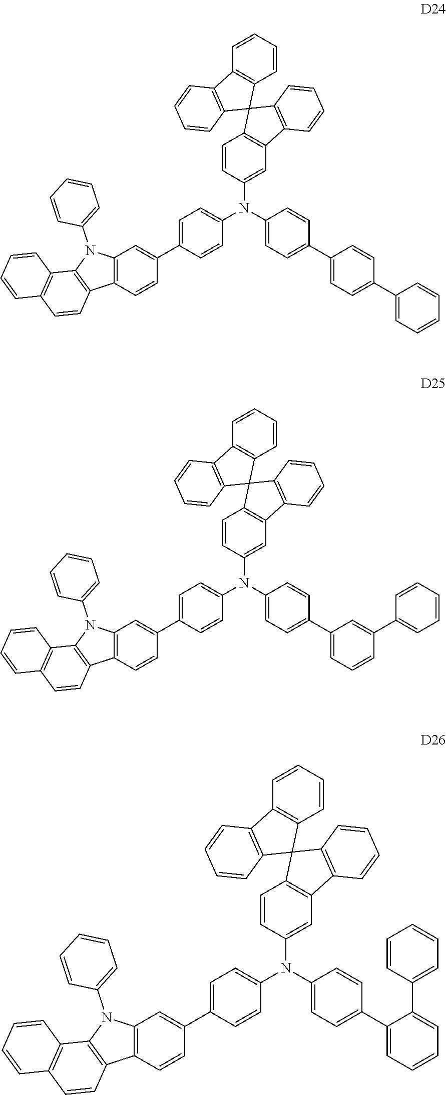 [SMILES: c1ccc(-c2ccc(-c3ccc(N(c4ccc(-c5ccc6c7ccc8ccccc8c7n(-c7ccccc7)c6c5)cc4)c4ccc5c(c4)-c4ccccc4C54c5ccccc5-c5ccccc54)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)-c4ccccc4C54c5ccccc5-c5ccccc54)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2)cc1] 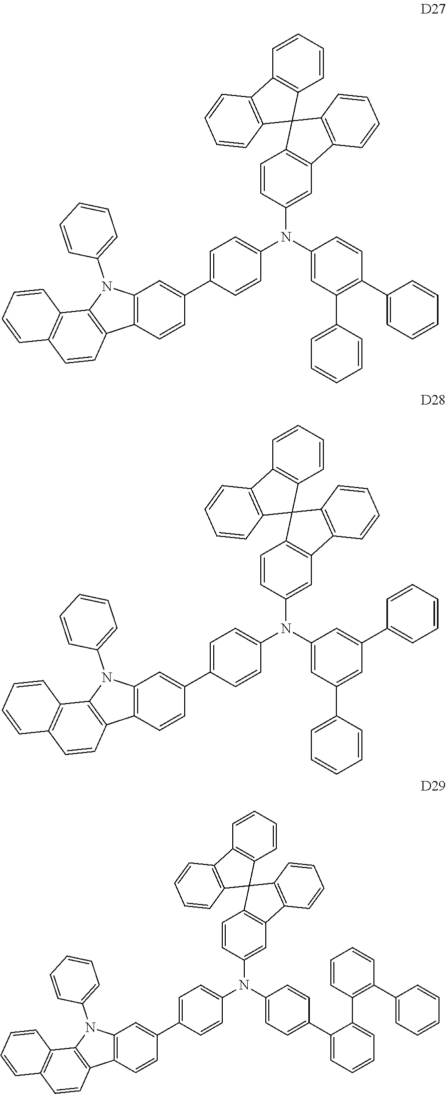 [SMILES: c1ccc(-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(c3)-c3ccccc3C43c4ccccc4-c4ccccc43)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)-c3ccccc3C43c4ccccc4-c4ccccc43)cc2)cc1] 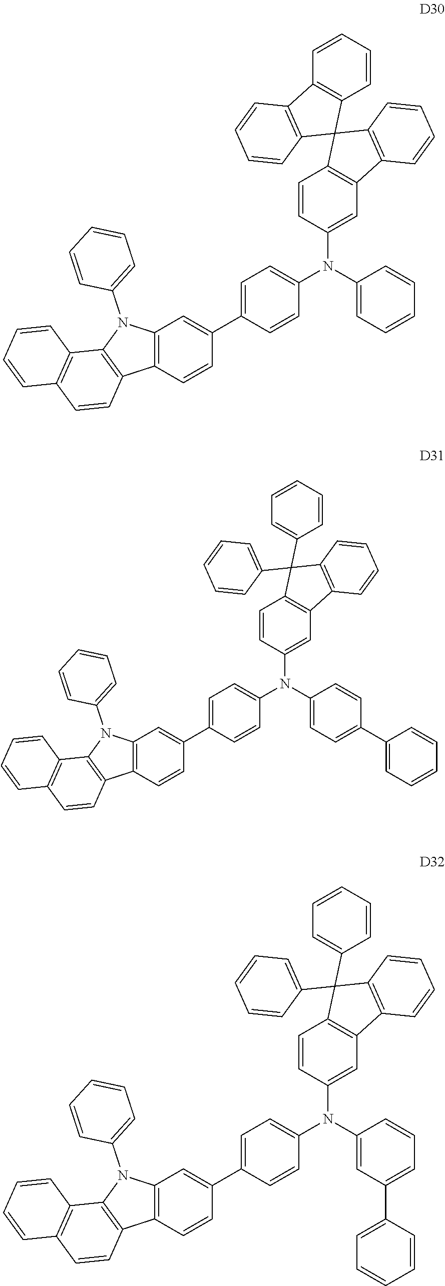 [SMILES: c1ccc(N(c2ccc(-c3ccc4c5ccc6ccccc6c5n(-c5ccccc5)c4c3)cc2)c2ccc3c(c2)-c2ccccc2C32c3ccccc3-c3ccccc32)cc1 ; c1ccc(-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2)cc1 ; c1ccc(-c2cccc(N(c3ccc(-c4ccc5c6ccc7ccccc7c6n(-c6ccccc6)c5c4)cc3)c3ccc4c(c3)-c3ccccc3C4(c3ccccc3)c3ccccc3)c2)cc1] 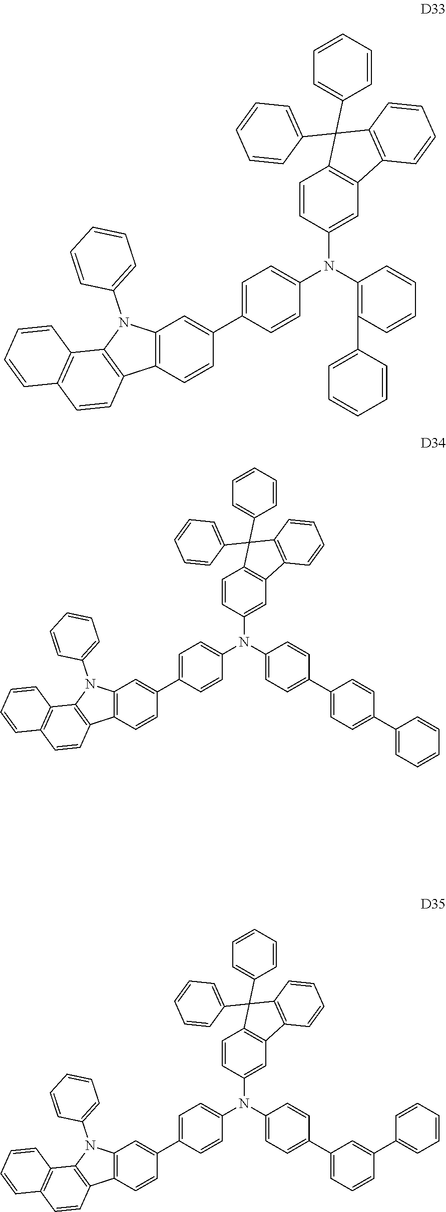 [SMILES: c1ccc(-c2ccccc2N(c2ccc(-c3ccc4c5ccc6ccccc6c5n(-c5ccccc5)c4c3)cc2)c2ccc3c(c2)-c2ccccc2C3(c2ccccc2)c2ccccc2)cc1 ; c1ccc(-c2ccc(-c3ccc(N(c4ccc(-c5ccc6c7ccc8ccccc8c7n(-c7ccccc7)c6c5)cc4)c4ccc5c(c4)-c4ccccc4C5(c4ccccc4)c4ccccc4)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)-c4ccccc4C5(c4ccccc4)c4ccccc4)cc3)c2)cc1] 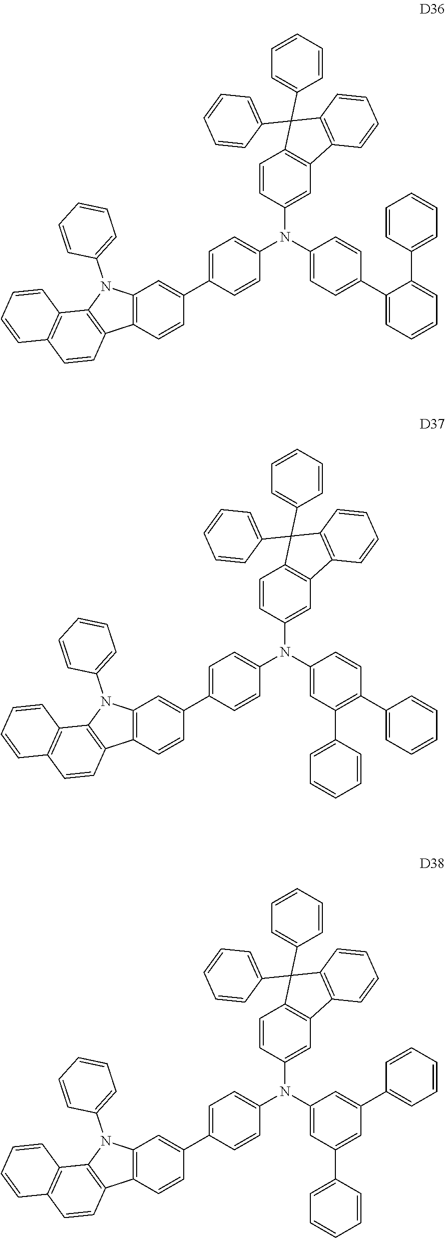 [SMILES: c1ccc(-c2ccccc2-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2)cc1 ; c1ccc(-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(c3)-c3ccccc3C4(c3ccccc3)c3ccccc3)c2)cc1] 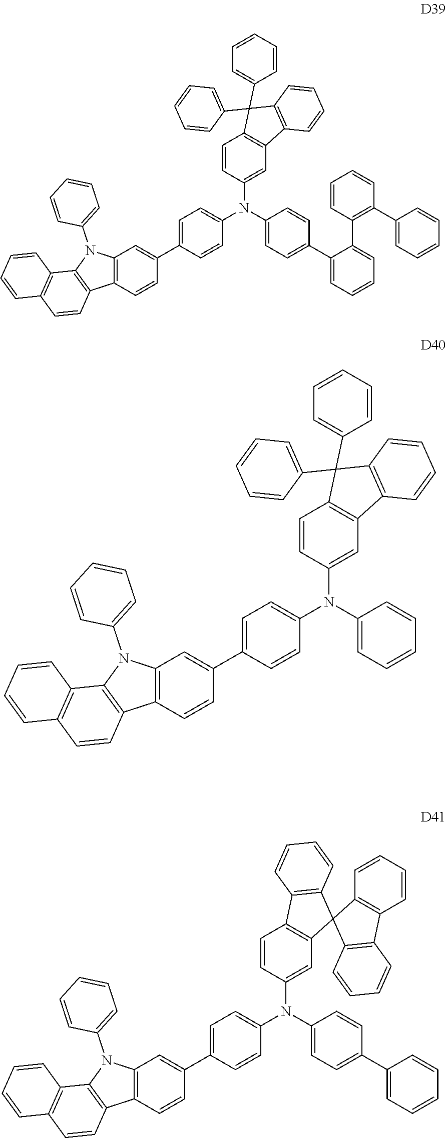 [SMILES: c1ccc(-c2ccccc2-c2ccccc2-c2ccc(N(c3ccc(-c4ccc5c6ccc7ccccc7c6n(-c6ccccc6)c5c4)cc3)c3ccc4c(c3)-c3ccccc3C4(c3ccccc3)c3ccccc3)cc2)cc1 ; c1ccc(N(c2ccc(-c3ccc4c5ccc6ccccc6c5n(-c5ccccc5)c4c3)cc2)c2ccc3c(c2)-c2ccccc2C3(c2ccccc2)c2ccccc2)cc1 ; c1ccc(-c2ccc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)cc2)cc1] 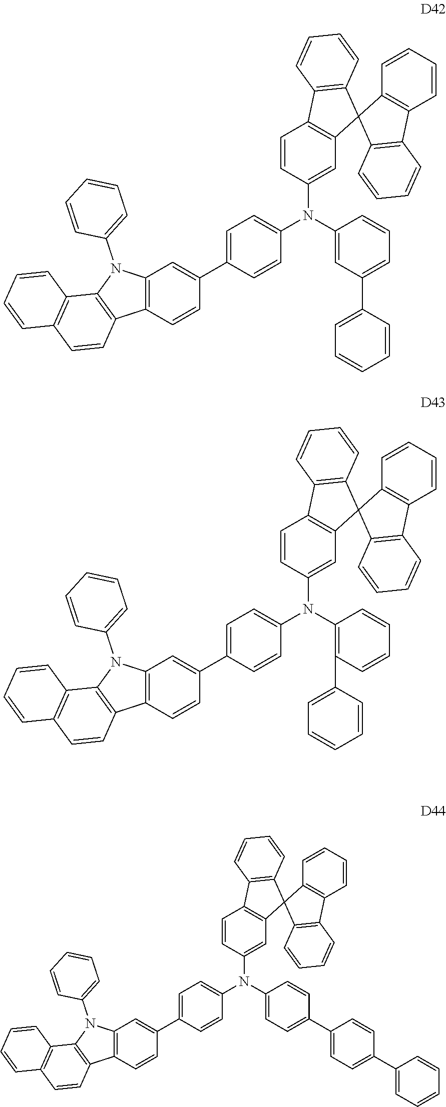 [SMILES: c1ccc(-c2cccc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)c2)cc1 ; c1ccc(-c2ccccc2N(c2ccc(-c3ccc4c5ccc6ccccc6c5n(-c5ccccc5)c4c3)cc2)c2ccc3c(c2)C2(c4ccccc4-c4ccccc42)c2ccccc2-3)cc1 ; c1ccc(-c2ccc(-c3ccc(N(c4ccc(-c5ccc6c7ccc8ccccc8c7n(-c7ccccc7)c6c5)cc4)c4ccc5c(c4)C4(c6ccccc6-c6ccccc64)c4ccccc4-5)cc3)cc2)cc1] 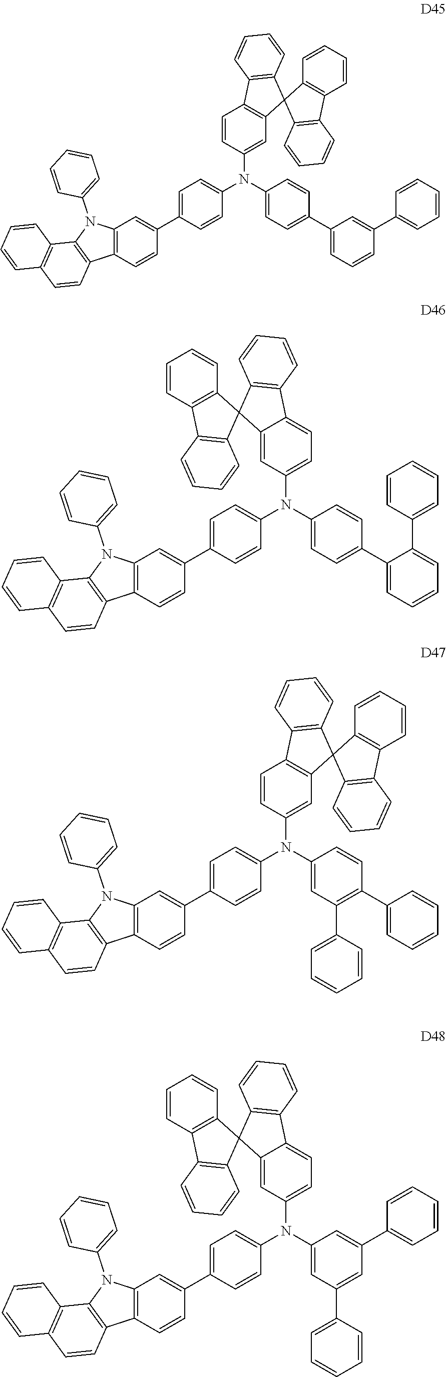 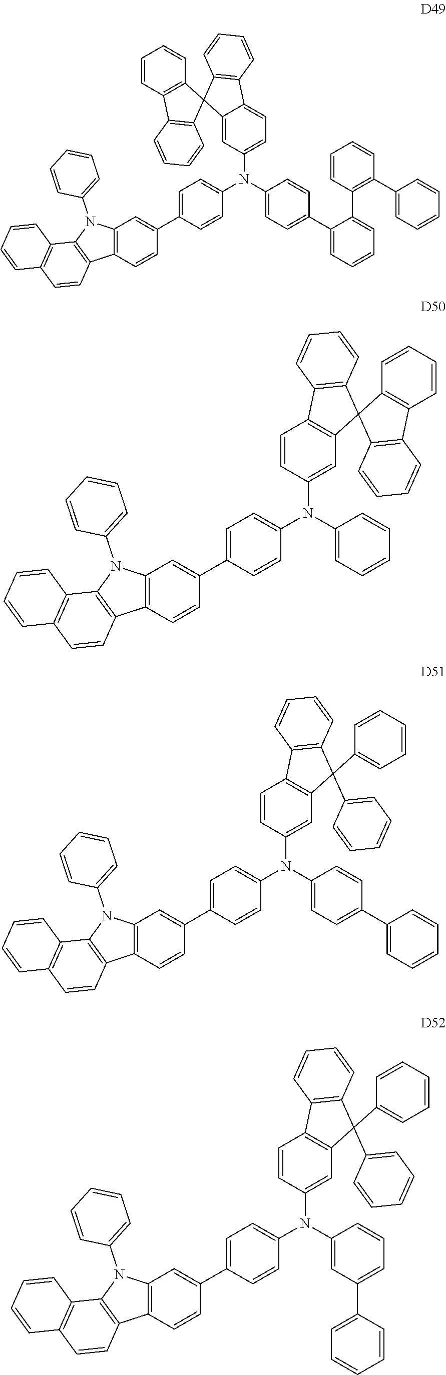 [SMILES: c1ccc(-c2ccccc2-c2ccccc2-c2ccc(N(c3ccc(-c4ccc5c6ccc7ccccc7c6n(-c6ccccc6)c5c4)cc3)c3ccc4c(c3)C3(c5ccccc5-c5ccccc53)c3ccccc3-4)cc2)cc1 ; c1ccc(N(c2ccc(-c3ccc4c5ccc6ccccc6c5n(-c5ccccc5)c4c3)cc2)c2ccc3c(c2)C2(c4ccccc4-c4ccccc42)c2ccccc2-3)cc1 ; c1ccc(-c2ccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)cc2)cc1 ; c1ccc(-c2cccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)c2)cc1] 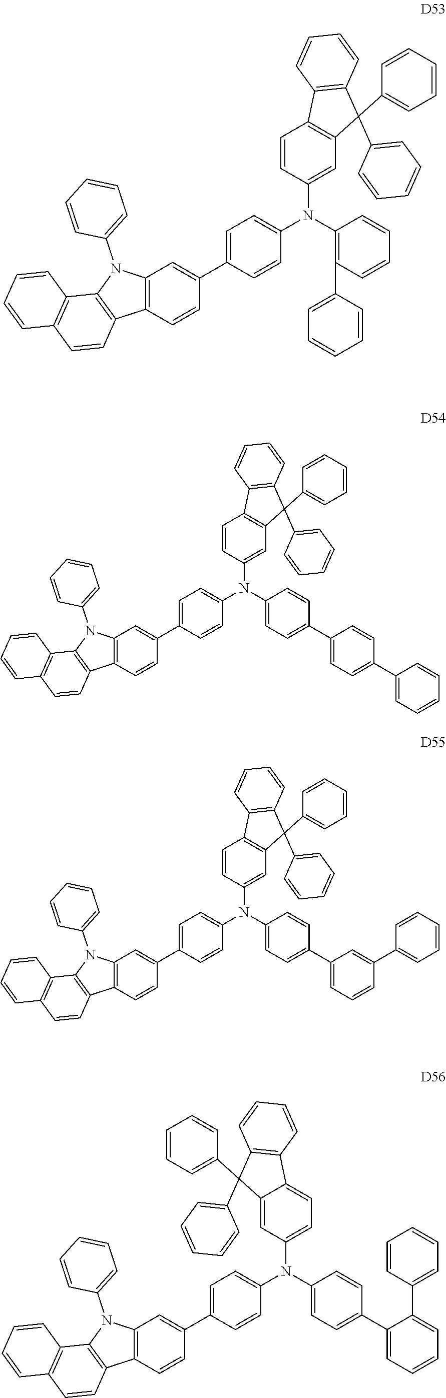 [SMILES: c1ccc(-c2ccccc2N(c2ccc(-c3ccc4c5ccc6ccccc6c5n(-c5ccccc5)c4c3)cc2)c2ccc3c(c2)C(c2ccccc2)(c2ccccc2)c2ccccc2-3)cc1 ; c1ccc(-c2ccc(-c3ccc(N(c4ccc(-c5ccc6c7ccc8ccccc8c7n(-c7ccccc7)c6c5)cc4)c4ccc5c(c4)C(c4ccccc4)(c4ccccc4)c4ccccc4-5)cc3)cc2)cc1 ; c1ccc(-c2cccc(-c3ccc(N(c4ccc(-c5ccc6c7ccc8ccccc8c7n(-c7ccccc7)c6c5)cc4)c4ccc5c(c4)C(c4ccccc4)(c4ccccc4)c4ccccc4-5)cc3)c2)cc1 ; c1ccc(-c2ccccc2-c2ccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)cc2)cc1] 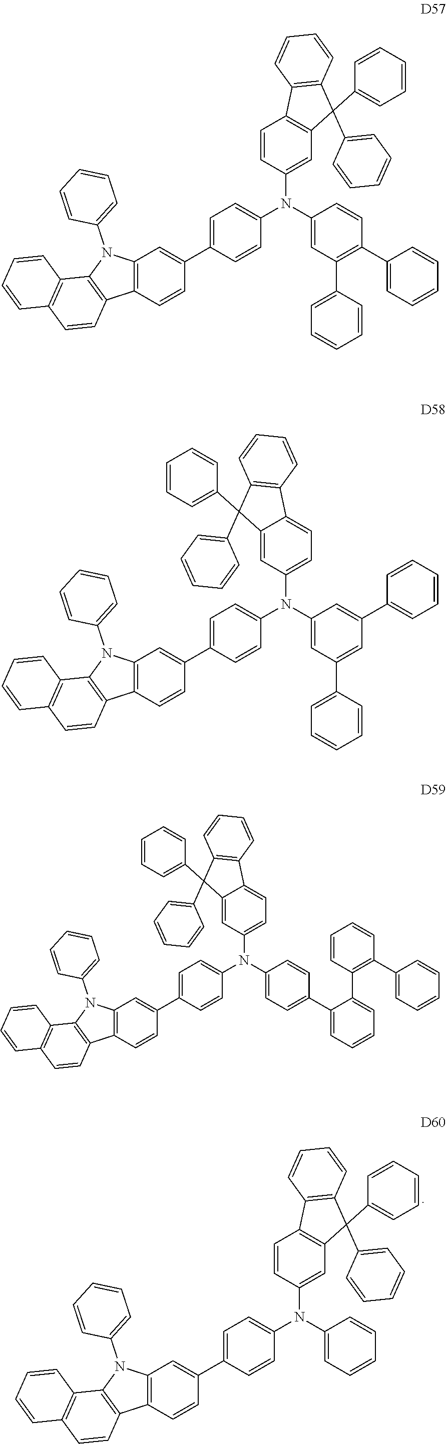 [SMILES: c1ccc(-c2ccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)cc2-c2ccccc2)cc1 ; c1ccc(-c2cc(-c3ccccc3)cc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)c2)cc1 ; c1ccc(-c2ccccc2-c2ccccc2-c2ccc(N(c3ccc(-c4ccc5c6ccc7ccccc7c6n(-c6ccccc6)c5c4)cc3)c3ccc4c(c3)C(c3ccccc3)(c3ccccc3)c3ccccc3-4)cc2)cc1 ; c1ccc(N(c2ccc(-c3ccc4c5ccc6ccccc6c5n(-c5ccccc5)c4c3)cc2)c2ccc3c(c2)C(c2ccccc2)(c2ccccc2)c2ccccc2-3)cc1]
